(19) United States
(12) Patent Application Publication
(10) Pub. No.: US 2026/0022143 A1
(43) Pub. Date: Jan. 22, 2026

(54) Cyclic Peptide Inhibitors of IL-23

(71) Applicant: JANSSEN PHARMACEUTICA NV, Beerse (BE)

(72) Inventors: Stephanie Barros, Chalfont, PA (US); Boying Guo, Warrington, PA (US); Charles Hendrick, North Wales, PA (US); David Marcoux, Newton Centre, MA (US); Santhosh Neelamkavil, Edison, NJ (US); Sandeep Somani, Princeton, NJ (US); Chengzao Sun, Warrington, PA (US); Tho Van Thieu, North Wales, PA (US); Jing Zhang, Wilmington, DE (US); Raymond J. Patch, Yardley, PA (US); Elisabetta Bianchi, Roma (IT); Danila Branca, Pomezia (IT); Gianni Cavallo, Lesina (FG) (IT); Roberto Costante, Pomezia (IT); Raffaele Ingenito, Pomezia (IT); Antonia Rinaldi, Valsinni (MT) (IT)

(21) Appl. No.: 18/993,874

(22) PCT Filed: Jul. 14, 2023
(86) PCT No.: PCT/US2023/070202

§ 371 Date: Jan. 13, 2025

Related U.S. Application Data
(60) Provisional application No. 63/389,242, filed on Jul. 14, 2022.

(51) Int. Cl. C07K 7/64; A61K 38/00

(52) U.S. Cl. CPC C07K 7/64; A61K 38/00

(57) ABSTRACT

The present invention relates to novel cyclic peptide inhibitors of the interleukin-23 receptor (IL-23R) or pharmaceutically acceptable salt thereof, corresponding pharmaceutical compositions, methods and/or uses for treatment of autoimmune inflammation and related diseases and disorders.

### CROSS REFERENCE TO RELATED APPLICATIONS

This application is a 35 U.S.C. § 371 filing of International Patent Application No. PCT/US2023/070202, filed Jul. 14, 2023, which claims priority to U.S. Patent Application No. 63/389,242, filed Jul. 14, 2022, the contents of each of which are incorporated herein by reference in their entirety.

### FIELD OF THE INVENTION

The present invention relates to novel peptide inhibitors of the interleukin-23 receptor (IL-23R) or pharmaceutically acceptable salts, solvates and/or other forms thereof, invention relates to corresponding pharmaceutical compositions, methods and/or uses of the IL-23R inhibitors for treatment of autoimmune inflammation diseases and/or related disorders.

### REFERENCE TO SEQUENCE LISTING SUBMITTED ELECTRONICALLY

This application contains a sequence listing, which is submitted electronically via The United States Patent and Trademark Center Patent Center as an XML formatted sequence listing with a file name “760766_NTT-6738US_SL.xml” and a creation date of Mar. 19, 2025, and having a size of 3,262,341 bytes. The sequence listing submitted via Patent Center is part of the specification and is herein incorporated by reference.

### BACKGROUND

The interleukin-23 (IL-23) cytokine has been implicated as playing a crucial role in the pathogenesis of autoimmune inflammation and related diseases and disorders, such as multiple sclerosis, asthma, rheumatoid arthritis, psoriasis, and inflammatory bowel diseases (IBDs), for example, ulcerative colitis and Crohn's disease. Studies in acute and chronic mouse models of IBD revealed a primary role of interleukin-23 receptor (IL-23R) and downstream effector cytokines in disease pathogenesis. IL-23R is expressed on various adaptive and innate immune cells including Th17 cells, γδ T cells, natural killer (NK) cells, dendritic cells, macrophages, and innate lymphoid cells, which are found abundantly in the intestine. At the intestine mucosal surface, the gene expression and protein levels of IL-23R are found to be elevated in IBD patients. It is believed that IL-23 mediates this effect by promoting the development of a pathogenic CD4⁺ T cell population that produces IL-6, IL-17, and tumor necrosis factor (TNF).

Production of IL-23 is enriched in the intestine, where it is believed to play a key role in regulating the balance between tolerance and immunity through T-cell-dependent and T-cell-independent pathways of intestinal inflammation through effects on T-helper 1 (Th1) and Th17-associated cytokines, as well as restraining regulatory T-cell responses in the gut, favoring inflammation. In addition, polymorphisms in the IL-23 receptor (IL-23R) have been associated with susceptibility to inflammatory bowel diseases (IBDs), further establishing the critical role of the IL-23 pathway in intestinal homeostasis.

Psoriasis, a chronic skin disease affecting about 2%-3% of the general population has been shown to be mediated by the body's T cell inflammatory response mechanisms. IL-23 has one of several interleukins implicated as a key player in the pathogenesis of psoriasis, purportedly by maintaining chronic autoimmune inflammation via the induction of interleukin-17, regulation of T memory cells, and activation of macrophages. Expression of IL-23 and IL-23R has been shown to be increased in tissues of patients with psoriasis, and antibodies that neutralize IL-23 showed IL-23-dependent inhibition of psoriasis development in animal models of psoriasis.

IL-23 is a heterodimer composed of a unique p19 subunit and the p40 subunit shared with IL-12, which is a cytokine involved in the development of interferon-γ (IFN-γ)-producing T helper 1 (T_{H}1) cells. Although IL-23 and IL-12 both contain the p40 subunit, they have different phenotypic properties. For example, animals deficient in IL-12 are susceptible to inflammatory autoimmune diseases, whereas IL-23 deficient animals are resistant, presumably due to a reduced number of CD4⁺ T cells producing IL-6, IL-17, and TNF in the CNS of IL-23-deficient animals. IL-23 binds to IL-23R, which is a heterodimeric receptor composed of IL-12Rβ1 and IL-23R subunits. Binding of IL-23 to IL-23R activates the Jak-Stat signaling molecules, Jak2, Tyk2, and Stat1, Stat 3, Stat 4, and Stat 5, although Stat4 activation is substantially weaker and different DNA-binding Stat complexes form in response to IL-23 as compared with IL-12. IL-23R associates constitutively with Jak2 and in a ligand-dependent manner with Stat3. In contrast to IL-12, which acts mainly on naive CD4(+) T cells, IL-23 preferentially acts on memory CD4(+) T cells.

Therapeutic moieties that inhibit the IL-23 pathway have been developed for use in treating IL-23-related diseases and disorders. A number of antibodies that bind to IL-23 or IL-23R have been identified, including ustekinumab, which has been approved for the treatment of moderate to severe plaque psoriasis (PSO), active psoriatic arthritis (PSA), moderately to severely active Crohn's disease (CD) and moderately to severely active ulcerative colitis (UC). Examples of such identified antibodies, include: Tildrakizumab, an anti-IL23 antibody approved for treatment of plaque psoriasis, Guselkumab, an anti-IL23 antibody approved for treatment of psoriatic arthritis and Risankizumab, an anti-IL23 antibody approved for the treatment of plaque psoriasis in the US, and generalized pustular psoriasis, erythrodermic psoriasis and psoriatic arthritis in Japan.

Although targeted TL-23 antibody therapeutics are used clinically, there are no small-molecule therapeutics that selectively inhibit TL-23 signaling. There are some identified polypeptide inhibitors that bind to TL-23R and inhibit binding of IL-23 to TL-23R (see, e.g., US Patent Application Publication No. US2013/0029907).Thus, there remains a significant need in the art for effective small-molecule and/or polypeptide therapeutic agents to treat and/or prevent IL-23-associated and/or IL23R-associated diseases and disorders.

### BRIEF SUMMARY

In general, the present invention relates to novel peptide inhibitors of the interleukin-23 receptor (TL-23R) or pharmaceutically acceptable salts, solvates and/or other forms thereof., corresponding pharmaceutical compositions, methods and/or uses of the TL-23R inhibitors for treatment of autoimmune inflammation diseases and/or related disorders.

In particular, the present invention relates to a cyclic peptide, or a pharmaceutically acceptable salt or solvate thereof, comprising an amino acid sequence of Formula (A):

(A)
Z³-Z⁴-Z⁵-Z⁶-Z⁷-Z⁸-Z⁹⁻¹⁰-Z¹¹-Z¹²-Z¹³-Z¹⁴-Z¹⁵-Z¹⁶,

wherein:
the amino acid residue at position Z³ is absent or the residue of r;
the amino acid residue at position Z⁴ is the residue of an amino acid that is connected to the amino acid residue at position Z⁹;
the amino acid residue at position Z⁵ is the residue of N(N(Me)₂);
the amino acid residue at position Z⁶ is the residue of T;
the amino acid residue at position Z⁷ is the residue of 7MeW;
the amino acid residue at position Z⁸ is the residue of K(NMeAc);
the amino acid residue at position Z⁹ is the residue of an amino acid that is connected to the amino acid residue at position Z⁴;
the amino acid residue at position Z¹⁰ is the residue of TMAPF;
the amino acid residue at position Z¹¹ is the residue of 2Nal;
the amino acid residue at position Z¹² is absent or the residue of THP;
the amino acid residue at position Z¹³ is the residue of K(NMeAc);
the amino acid residue at position Z¹⁴ is the residue of N;
the amino acid residue at position Z¹⁵ is absent or the residue of 3Pya;
the amino acid residue at position Z¹⁶ is absent or the residue of Sar;
eight or fewer of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position; and
at least one of the amino acid residues at positions Z³, Z⁵, Z⁶, Z⁷, Z⁸, Z¹⁰, Z¹¹, Z¹², Z¹³, Z¹⁴, Z¹⁵ and/or Z¹⁶, when an amino acid residue is present at the position, each independently comprise(s) at least one quaternary amine.

In particular, the present invention relates to a cyclic peptide, or a pharmaceutically acceptable salt or solvate thereof, comprising an amino acid sequence of Formula (B):

(B)
Z³-Z⁴-Z⁵-Z⁶-Z⁷-Z⁸-Z⁹-Z¹⁰-Z¹¹-Z¹²_Z¹³-Z¹⁴-Z¹⁵-Z¹⁶,

wherein:
the amino acid residue at position Z³ is absent or the residue of r;
the amino acid residue at position Z⁴ is the residue of an amino acid that is connected to the amino acid residue at position Z⁹;
the amino acid residue at position Z⁵ is the residue of N(N(Me)₂);
the amino acid residue at position Z⁶ is the residue of T;
the amino acid residue at position Z⁷ is the residue of 7MeW;
the amino acid residue at position Z⁸ is the residue of K(NMeAc);
the amino acid residue at position Z⁹ is the residue of an amino acid that is connected to the amino acid residue at position Z⁴;
the amino acid residue at position Z¹⁰ is the residue of TMAPF;
the amino acid residue at position Z¹¹ is the residue of 2Nal;
the amino acid residue at position Z¹² is absent or the residue of THP;
the amino acid residue at position Z¹³ is the residue of K(NMeAc);
the amino acid residue at position Z¹⁴ is the residue of N;
the amino acid residue at position Z¹⁵ is absent or the residue of 3Pya;
the amino acid residue at position Z¹⁶ is absent or the residue of Sar; eight or fewer of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position; and
at least one of the amino acid residues at positions Z³, Z⁵, Z⁶, Z⁷, Z⁸, Z¹⁰, Z¹¹, Z¹², Z¹³, Z¹⁴, Z¹⁵ and/or Z¹⁶, when an amino acid residue is present at the position, each independently comprise(s) at least one masked amine and/or masked amide.

In particular, the present invention relates to a cyclic peptide, or a pharmaceutically acceptable salt or solvate thereof, comprising an amino acid sequence of Formula (C):

(C)
Z³-Z⁴-Z⁵-Z⁶-Z⁷-Z⁸-Z⁹-Z¹⁰-Z¹¹-Z¹²-Z¹³-Z¹⁴-Z¹⁵-Z¹⁶,

wherein:
the amino acid residue at position Z³ is absent or the residue of r;
the amino acid residue at position Z⁴ is the residue of an amino acid that is connected to the amino acid residue at position Z⁹;
the amino acid residue at position Z⁵ is the residue of N(N(Me)₂);
the amino acid residue at position Z⁶ is the residue of T;
the amino acid residue at position Z⁷ is the residue of 7MeW;
the amino acid residue at position Z⁸ is the residue of K(NMeAc);
the amino acid residue at position Z⁹ is the residue of an amino acid that is connected to the amino acid residue at position Z⁴;
the amino acid residue at position Z¹⁰ is the residue of TMAPF;
the amino acid residue at position Z¹¹ is the residue of 2Nal;
the amino acid residue at position Z¹² is absent or the residue of THP;
the amino acid residue at position Z¹³ is the residue of K(NMeAc);
the amino acid residue at position Z¹⁴ is the residue of N;
the amino acid residue at position Z¹⁵ is absent or the residue of 3Pya;
the amino acid residue at position Z¹⁶ is absent or the residue of Sar; and
eight or fewer of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

The present invention also relates to a compound which is selected from any one of the tables 1A, 1B, 1C, 1D, 1E and 1 F or pharmaceutically acceptable salts thereof or solvates thereof.

In particular, the present invention relates to a compound of Formulae (I) to (X), or pharmaceutically acceptable salts, solvates and/or other forms thereof. corresponding pharmaceutical compositions, methods and/or uses for treatment of autoimmune inflammation diseases and related disorders.

The present invention relates to novel peptide inhibitors of the interleukin-23 receptor (IL-23R) of Formulae (I) to (VI) or pharmaceutically acceptable salts, solvates and/or other forms thereof, invention relates to corresponding pharmaceutical compositions, methods and/or uses of the IL-23R inhibitors for treatment of autoimmune inflammation diseases and/or related disorders.

The present invention also relates to compounds set forth in any of Tables 1A-H, or pharmaceutically acceptable salts, solvates, or forms thereof, corresponding pharmaceutical compositions, and methods and/or uses for treatment of autoimmune inflammation diseases and related disorders.

The present disclosure also relates to pharmaceutical composition(s), which comprises a herein-described peptide inhibitor compound of the or a pharmaceutically acceptable salt, solvate, or form thereof as described herein, and a pharmaceutically acceptable carrier, excipient, or diluent. The pharmaceutical compositions may comprise or may exclude an absorption enhancer depending on the intended route of delivery or use thereof for treatment of specific indications. The absorption enhancer may be permeation enhancer or intestinal permeation enhancer. In an aspect the absorption enhancer improves oral bioavailability.

The present invention relates to method(s) for treating and/or uses(s) for inflammatory disease(s) in a subject, which comprises administering a therapeutically effective amount of one or more herein-described peptide inhibitor compounds of the IL-23R or pharmaceutically acceptable salts, or solvates thereof, or a corresponding pharmaceutical composition as described herein, respectively to a subject in need thereof. Such inflammatory diseases and related disorders may include, but are not limited to, inflammatory bowel disease (IBD), Crohn's disease (CD), ulcerative colitis (UC), psoriasis (PsO), or psoriatic arthritis (PsA) and the like.

The present inventionprovides for the use of one or more herein-described compounds (e.g., compounds of Formulae (I) to (X) or Tables 1A to 1H) for the preparation of pharmaceutical compositions for use in the treatment of inflammatory diseases and related disorders including, but not limited to, inflammatory bowel disease (IBD), Crohn's disease (CD), ulcerative colitis (UC), psoriasis (PsO), and psoriatic arthritis (PsA).

The present inventionprovides for the use of one or more herein-described compounds of Formulae (I) to (X) or Tables 1A to 1H in the treatment of inflammatory diseases and related disorders including, but not limited to, inflammatory bowel disease (IBD), Crohn's disease (CD), ulcerative colitis (UC), psoriasis (PsO), and psoriatic arthritis (PsA).

The present inventionprovides for kits comprising one or more herein-described compounds of Formulae (I) to (X) or Tables 1A to 1H and instructions for use in treating an a disease in a patient. The disease may be an inflammatory diseases or related disorder including, but not limited to, inflammatory bowel disease (IBD), Crohn's disease (CD), ulcerative colitis (UC), psoriasis (PsO), and psoriatic arthritis (PsA).

### DETAILED DESCRIPTION

### I. GENERAL

The cyclic peptides of the present invention comprise quaternary amines, masked amines, masked amides or combinations thereof. The inclusion of quaternary amines, masked amines, and/or masked amides in the cyclic peptides of the present invention has improved the pharamacokinetic properties of the cyclic peptides compared to other compounds that target IL-23R. For example, the cyclic peptides of the present invention have highly desirable oral PK profiles and improved oral bioavaiability. The cyclic peptides of the present invention also have lower projected human doses. It is contemplated that the quaternary amines and masked amines and/or masked amides achieve these improvements by changing the polarity propreties of the molecules as reflected in the observed Exposed Polar Surface Area (EPSA) values.

The present invention relates to novel peptide inhibitors of the IL-23R or pharmaceutically acceptable salts, solvates, or forms thereof, corresponding pharmaceutical compositions, methods and/or uses for treatment of autoimmune inflammation and related diseases and disorders.

The present invention provides or relates to peptide inhibitors of an IL-23R. The peptide inhibitors of the present invention may exhibit enhanced properties, such as longer in vivo half-life, compared to the corresponding cyclic peptide inhibitor of an IL-23R without a cyclic structure. In particular, compounds and methods for specific targeting of IL-23R from the luminal side of the gut may provide therapeutic benefit to IBD patients suffering from local inflammation of the intestinal tissue; and/or orally bioavailable small molecule and/or polypeptide inhibitors of IL-23 may provide both a non-steroidal treatment option for patients with mild to moderate psoriasis and treatment for moderate to severe psoriasis that does not require delivery by infusion.

Compounds and methods for specific targeting of the IL-23R from the luminal side of the gut may provide therapeutic benefit to IBD patients suffering from local inflammation of the intestinal tissue. In addition, orally bioavailable small molecule and/or polypeptide inhibitors of IL-23 may provide both a non-steroidal treatment option for patients with mild to moderate psoriasis and treatment for moderate to severe psoriasis that does not require delivery by infusion.

The present invention is directed to addressing these needs by providing peptide inhibitors or pharmaceutically acceptable salts, solvates and/or other forms thereof, that bind IL-23R to inhibit IL-23 binding and signaling, via different suitable routes of administration, which may include but is not limited to oral administration.

In one general aspect, the invention relates to a cyclic peptide, or a pharmaceutically acceptable salt or solvate thereof, comprising an amino acid sequence of Formula (A):

(A)
Z³-Z⁴-Z⁵-Z⁶-Z⁷-Z⁸-Z⁹-Z¹⁰-Z¹¹-Z¹²-Z¹³-Z¹⁴-Z¹⁵-Z¹⁶,

wherein:
the amino acid residue at position Z³ is absent or the residue of r;
the amino acid residue at position Z⁴ is the residue of an amino acid that is connected to the amino acid residue at position Z⁹;
the amino acid residue at position Z⁵ is the residue of N(N(Me)₂);
the amino acid residue at position Z⁶ is the residue of T;
the amino acid residue at position Z⁷ is the residue of 7MeW;
the amino acid residue at position Z⁸ is the residue of K(NMeAc);
the amino acid residue at position Z⁹ is the residue of an amino acid that is connected to the amino acid residue at position Z⁴;
the amino acid residue at position Z¹⁰ is the residue of TMAPF;
the amino acid residue at position Z¹¹ is the residue of 2Nal;
the amino acid residue at position Z¹² is absent or the residue of THP;
the amino acid residue at position Z¹³ is the residue of K(NMeAc);
the amino acid residue at position Z¹⁴ is the residue of N;
the amino acid residue at position Z¹⁵ is absent or the residue of 3Pya;
the amino acid residue at position Z¹⁶ is absent or the residue of Sar;
eight or fewer of the amino acid residues at positions Z³, Z⁵, Z⁶, Z⁷, Z⁸, Z¹⁰,, ¹¹, Z¹², Z¹³, Z¹⁴, Z¹⁵ and Z¹⁶, when an amino acid residue is present at the position, are independently replaced with an amino acid residue that is different from the amino acid residue recited at said position; and
at least one of the amino acid residues at positions Z³, Z⁵, Z⁶, Z⁷, Z⁸, Z¹⁰, Z¹¹, Z¹², Z¹³, Z¹⁴, Z¹⁵ and/or Z¹⁶, when an amino acid residue is present at the position, each independently comprise(s) at least one quaternary amine.

In another general aspect, the invention relates to a cyclic peptide, or a pharmaceutically acceptable salt or solvate thereof, comprising an amino acid sequence of Formula (B):

(B)
Z³-Z⁴-Z⁵-Z⁶-Z⁷-Z⁸-Z⁹-Z¹⁰-Z¹¹-Z¹²-Z¹³-Z¹⁴-Z¹⁵-Z¹⁶,

wherein:
the amino acid residue at position Z³ is absent or the residue of r;
the amino acid residue at position Z⁴ is the residue of an amino acid that is connected to the amino acid residue at position Z⁹;
the amino acid residue at position Z⁵ is the residue of N(N(Me)₂);
the amino acid residue at position Z⁶ is the residue of T;
the amino acid residue at position Z⁷ is the residue of 7MeW;
the amino acid residue at position Z⁸ is the residue of K(NMeAc);
the amino acid residue at position Z⁹ is the residue of an amino acid that is connected to the amino acid residue at position Z⁴;
the amino acid residue at position Z¹⁰ is the residue of TMAPF;
the amino acid residue at position Z¹¹ is the residue of 2Nal;
the amino acid residue at position Z¹² is absent or the residue of THP;
the amino acid residue at position Z¹³ is the residue of K(NMeAc);
the amino acid residue at position Z¹⁴ is the residue of N;
the amino acid residue at position Z¹⁵ is absent or the residue of 3Pya;
the amino acid residue at position Z¹⁶ is absent or the residue of Sar;
eight or fewer of the amino acid residues at positions Z³, Z⁵, Z⁶, Z⁷, Z⁸, Z¹⁰, Z¹¹, Z¹², Z¹³, Z⁴, Z⁵ and Z¹⁶, when an amino acid residue is present at the position, are independently replaced with an amino acid residue that is different from the amino acid residue recited at said position; and
at least one of the amino acid residues at positions Z³, Z⁵, Z⁶, Z⁷, Z⁸, Z¹⁰, Z¹¹, Z¹², Z¹³, Z¹⁴, Z⁵ and/or Z¹⁶, when an amino acid residue is present at the position, each independently comprise(s) at least one masked amine and/or masked amide

In another general aspect, the invention relates to a cyclic peptide, or a pharmaceutically acceptable salt or solvate thereof, comprising an amino acid sequence of Formula (C):

(C)
Z³-Z⁴-Z⁵-Z⁶-Z⁷-Z⁸-Z⁹-Z¹⁰-Z¹¹-Z¹²-Z¹³-Z¹⁴-Z¹⁵-Z¹⁶,

wherein:
the amino acid residue at position Z³ is absent or the residue of r;
the amino acid residue at position Z⁴ is the residue of an amino acid that is connected to the amino acid residue at position Z⁹;
the amino acid residue at position Z⁵ is the residue of N(N(Me)₂);
the amino acid residue at position Z⁶ is the residue of T;
the amino acid residue at position Z⁷ is the residue of 7MeW;
the amino acid residue at position Z⁸ is the residue of K(NMeAc);
the amino acid residue at position Z⁹ is the residue of an amino acid that is connected to the amino acid residue at position Z⁴;
the amino acid residue at position Z¹⁰ is the residue of TMAPF;
the amino acid residue at position Z¹¹ is the residue of 2Nal;
the amino acid residue at position Z¹² is absent or the residue of THP;
the amino acid residue at position Z¹³ is the residue of K(NMeAc);
the amino acid residue at position Z¹⁴ is the residue of N;
the amino acid residue at position Z¹⁵ is absent or the residue of 3Pya;
the amino acid residue at position Z¹⁶ is absent or the residue of Sar; and
eight or fewer of the amino acid residues at positions Z³, Z⁵, Z⁶, Z⁷, Z⁸, Z¹⁰, Z¹¹, Z¹², Z¹³, Z⁴, Z⁵ and Z¹⁶, when an amino acid residue is present at the position, are independently replaced with an amino acid residue that is different from the amino acid residue recited at said position.

In some embodiments, the amino acid residue at position Z⁴ is the residue of Abu or the residue of an amino acid comprising a sulfhydryl group.

In some embodiments, the amino acid residue at position Z⁹ is the residue of an amino acid comprising a sulfhydryl group.

In some embodiments, when the amino acid residue at position Z⁴ is the residue of an amino acid comprising a sulfhydryl group, the amino acid residue at position Z⁴ is connected to the amino acid residue at position Z⁹ by a disulfide bond formed between the amino acid comprising a sulfhydryl group at position Z⁴ and the amino acid comprising a sulfhydryl group at position Z⁹.

In some embodiments, the amino acid residue at position Z⁴ is the residue of an amino acid comprising a sulfhydryl group, and the amino acid residue at position Z⁴ is connected to the amino acid residue at position Z⁹ by a disulfide bond formed between the amino acid comprising a sulfhydryl group at position Z⁴ and the amino acid comprising a sulfhydryl group at position Z⁹.

In some embodiments, when the amino acid residue at position Z⁴ is the residue of Abu, the amino acid residue at position Z⁴ is connected to the amino acid residue at position Z⁹ by a thioether bond formed between the Abu at position Z⁴ and the amino acid comprising a sulfhydryl group at position Z⁹.

In some embodiments, the amino acid residue at position Z⁴ is the residue of Abu, and the amino acid residue at position Z⁴ is connected to the amino acid residue at position Z⁹ by a thioether bond formed between the Abu at position Z⁴ and the amino acid comprising a sulfhydryl group at position Z⁹.

In some embodiments, the cyclic peptide further comprises R^{NT}, wherein R^{NT} is bound to the N-terminal amine of the amino acid residue at position:
(i) Z³ when Z³ is present, or
(ii) Z⁴ when Z³ is absent; and
R^{NT} is selected from the group consisting of: —C(O)-optionally substituted (C₁-C₂₀) alkyl and —C(O)-optionally substituted (C₁-C₄₀) heteroalkyl.

In some embodiments, the cyclic peptide further comprises R^{CT}, wherein R^{CT} is bound to the carbonyl derived from the C-terminal carboxylic acid of the amino acid residue at position:
(i) Z¹⁶ when Z¹⁶ is present,
(ii) Z¹⁵ when Z¹⁶ is absent, or
(iii) Z¹⁴ when Z¹⁵ and Z¹⁶ are absent; and
R^{CT} is —N(R^{Y})(R^{Z}), wherein:
(i) each R^{Y} and R^{Z} is independently selected from the group consisting of: hydrogen, optionally substituted (C₁-C₁₅) alkyl and optionally substituted (C₁-C₃₀) heteroalkyl, or
(ii) each R^{Y} and R^{Z} come together with the N atom to which they are attached to form an optionally substituted (C₃-C₁₄) heterocyclic ring or an optionally substituted (C₅-C₁₀) bicyclic heterocyclic ring.

In some embodiments, the cyclic peptide further comprises R^{CT}, wherein R^{CT} is bound to the carbonyl derived from the C-terminal carboxylic acid of the amino acid residue at position:
(i) Z¹⁶ when Z¹⁶ is present,
(ii) Z¹⁵ when Z¹⁶ is absent, or
(iii) Z¹⁴ when Z¹⁵ and Z¹⁶ are absent; and
R^{CT} is —N(R^{Y})(R^{Z}), wherein:
(i) each R^{Y} and R^{Z} is independently selected from the group consisting of: hydrogen, optionally substituted (C₁-C₁₅) alkyl and optionally substituted (C₁-C₃₀) heteroalkyl, provided that only one of R and R^{Z} is hydrogen, or
(ii) each R^{Y} and R^{Z} come together with the N atom to which they are attached to form an optionally substituted (C₃-C₁₄) heterocyclic ring or an optionally substituted (C₅-C₁₀) bicyclic heterocyclic ring.

In some embodiments, the cyclic peptide further comprises R^{NT} and R^{CT} and is of Formula (D):

(D)
R^{NT}-Z³-Z⁴-Z⁵-Z⁶-Z⁷-Z⁸-Z⁹-Z¹⁰-Z¹¹-Z¹²-Z¹³-Z¹⁴-Z¹⁵-
Z¹⁶-R^{CT},

wherein R^{NT}, Z³, Z⁴, Z⁵, Z⁶, Z⁷, Z⁸, Z⁹, Z¹⁰, Z¹¹, Z¹², Z¹³, Z¹⁴, Z¹⁵, Z¹⁶ and R^{CT} are as defined herein.

In some embodiments, eight of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

In some embodiments, seven or fewer of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

In some embodiments, seven of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

In some embodiments, six or fewer of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

In some embodiments, six of the amino acid residues at positions Z³, Z⁵, Z⁶, Z⁷, Z⁸, Z¹⁰, Z¹¹, Z¹², Z¹³, Z⁴, Z⁵ and Z¹⁶, when an amino acid residue is present at the position, are independently replaced with an amino acid residue that is different from the amino acid residue recited at said position.

In some embodiments, five or fewer of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

In some embodiments, five of the amino acid residues at positions Z³, Z⁵, Z⁶, Z⁷, Z⁸, Z¹⁰, Z¹¹, Z¹², Z, Z¹⁴, Z¹⁵ and Z¹⁶, when an amino acid residue is present at the position, are independently replaced with an amino acid residue that is different from the amino acid residue recited at said position.

In some embodiments, four or fewer of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

In some embodiments, four of the amino acid residues at positions Z³, Z⁵, Z⁶, Z⁷, Z⁸, Z¹⁰, Z¹¹, Z¹², Z, Z¹⁴, Z¹⁵ and Z¹⁶, when an amino acid residue is present at the position, are independently replaced with an amino acid residue that is different from the amino acid residue recited at said position.

In some embodiments, three or fewer of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

In some embodiments, three of the amino acid residues at positions Z³, Z⁵, Z⁶, Z⁷, Z⁸, Z¹⁰, Z¹¹, Z¹², Z, Z¹⁴, Z¹⁵ and Z¹⁶, when an amino acid residue is present at the position, are independently replaced with an amino acid residue that is different from the amino acid residue recited at said position.

In some embodiments, two or fewer of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

In some embodiments, two of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

In some embodiments, one or fewer of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

In some embodiments, one of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, is independently replaced with an amino acid residue that is different from the amino acid residue recited at said position.

In some embodiments, none of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

In some embodiments, seven or fewer of the amino acid residues at positions Z³, Z⁵, Z⁸, Z¹⁰, Z¹³, Z¹⁵ and Z¹⁶, when an amino acid residue is present at the position, are independently replaced with an amino acid residue that is different from the amino acid residue recited at said position.

In some embodiments, seven of the amino acid residues at positions Z³, Z⁵, Z⁸, Z¹⁰, Z¹³, Z¹⁵ and Z¹⁶, when an amino acid residue is present at the position, are independently replaced with an amino acid residue that is different from the amino acid residue recited at said position.

In some embodiments, six or fewer of the amino acid residues at positions Z³, Z⁵, Z⁸, Z¹⁰, Z¹³, Z¹⁵ and Z¹⁶, when an amino acid residue is present at the position, are independently replaced with an amino acid residue that is different from the amino acid residue recited at said position.

In some embodiments, six of the amino acid residues at positions Z³, Z⁵, Z⁸, Z¹⁰, Z¹³, Z¹⁵ and Z¹⁶, when an amino acid residue is present at the position, are independently replaced with an amino acid residue that is different from the amino acid residue recited at said position.

In some embodiments, five or fewer of the amino acid residues at positions Z³, Z⁵, Z⁸, Z¹⁰, Z¹³, Z¹⁵ and Z¹⁶, when an amino acid residue is present at the position, are independently replaced with an amino acid residue that is different from the amino acid residue recited at said position.

In some embodiments, five of the amino acid residues at positions Z³, Z⁵, Z⁸, Z¹⁰, Z¹³, Z¹⁵ and Z¹⁶, when an amino acid residue is present at the position, are independently replaced with an amino acid residue that is different from the amino acid residue recited at said position.

In some embodiments, four or fewer of the amino acid residues at positions Z³, Z⁵, Z⁸, Z¹⁰, Z¹³, Z¹⁵ and Z¹⁶, when an amino acid residue is present at the position, are independently replaced with an amino acid residue that is different from the amino acid residue recited at said position.

In some embodiments, four of the amino acid residues at positions Z³, Z⁵, Z⁸, Z¹⁰, Z¹³, Z¹⁵ and Z¹⁶, when an amino acid residue is present at the position, are independently replaced with an amino acid residue that is different from the amino acid residue recited at said position.

In some embodiments, three or fewer of the amino acid residues at positions Z³, Z⁵, Z⁸, Z¹⁰, Z¹³, Z¹⁵ and Z¹⁶, when an amino acid residue is present at the position, are independently replaced with an amino acid residue that is different from the amino acid residue recited at said position.

In some embodiments, three of the amino acid residues at positions Z³, Z⁵, Z⁸, Z¹⁰, Z¹³, Z¹⁵ and Z¹⁶, when an amino acid residue is present at the position, are independently replaced with an amino acid residue that is different from the amino acid residue recited at said position.

In some embodiments, two or fewer of the amino acid residues at positions Z³, Z⁵, Z⁸, Z¹⁰, Z¹³, Z¹⁵ and Z¹⁶, when an amino acid residue is present at the position, are independently replaced with an amino acid residue that is different from the amino acid residue recited at said position.

In some embodiments, two of the amino acid residues at positions Z³, Z⁵, Z⁸, Z¹⁰, Z¹³, Z¹⁵ and Z¹⁶, when an amino acid residue is present at the position, are independently replaced with an amino acid residue that is different from the amino acid residue recited at said position.

In some embodiments, one or fewer of the amino acid residues at positions Z³, Z⁵, Z⁸, Z¹⁰, Z³, Z⁵ and Z¹⁶, when an amino acid residue is present at the position, are independently replaced with an amino acid residue that is different from the amino acid residue recited at said position.

In some embodiments, one of the amino acid residues at positions Z³, Z⁵, Z⁸, Z¹⁰, Z³, Z⁵ and Z¹⁶, when an amino acid residue is present at the position, is independently replaced with an amino acid residue that is different from the amino acid residue recited at said position.

In some embodiments, four or fewer of the amino acid residues at positions Z⁵, Z⁸, Z¹⁰, and Z¹³ are independently replaced with an amino acid residue that is different from the amino acid residue recited at said position.

In some embodiments, four of the amino acid residues at positions Z⁵, Z⁸, Z¹⁰, and Z¹³ are independently replaced with an amino acid residue that is different from the amino acid residue recited at said position.

In some embodiments, three or fewer of the amino acid residues at positions Z⁵, Z⁸, Z¹⁰, and Z¹³ are independently replaced with an amino acid residue that is different from the amino acid residue recited at said position.

In some embodiments, three of the amino acid residues at positions Z⁵, Z⁸, Z¹⁰, and Z¹³ are independently replaced with an amino acid residue that is different from the amino acid residue recited at said position.

In some embodiments, two or fewer of the amino acid residues at positions Z⁵, Z⁸, Z¹⁰, and Z¹³ are independently replaced with an amino acid residue that is different from the amino acid residue recited at said position.

In some embodiments, two of the amino acid residues at positions Z⁵, Z⁸, Z¹⁰, and Z¹³ are independently replaced with an amino acid residue that is different from the amino acid residue recited at said position.

In some embodiments, one or fewer of the amino acid residues at positions Z⁵, Z⁸, Z¹⁰, and Z¹³ are independently replaced with an amino acid residue that is different from the amino acid residue recited at said position.

In some embodiments, one of the amino acid residues at positions Z⁵, Z⁸, Z¹⁰, and Z¹³ is independently replaced with an amino acid residue that is different from the amino acid residue recited at said position.

In some embodiments, the amino acid residue at position Z³, when an amino acid residue is present at the position, is not replaced.

In some embodiments, the amino acid residue at position Z⁶ is not replaced.

In some embodiments, the amino acid residue at position Z⁷ is not replaced.

In some embodiments, the amino acid residue at position Z¹¹ is not replaced.

In some embodiments, the amino acid residue at position Z¹², when an amino acid residue is present at the position, is not replaced.

In some embodiments, the amino acid residue at position Z¹⁴ is not replaced.

In some embodiments, the amino acid residue at position Z¹⁵, when an amino acid residue is present at the position, is not replaced.

In some embodiments, the amino acid residue at position Z¹⁶, when an amino acid residue is present at the position, is not replaced.

In some embodiments, the amino acid residues at positions Z⁶ and Z¹¹ are not replaced.

In some embodiments, the amino acid residues at positions Z⁶ and Z¹⁴ are not replaced.

In some embodiments, the amino acid residues at positions Z¹¹ and Z¹⁴ are not replaced.

In some embodiments, the amino acid residues at positions Z⁶, Z¹¹ and Z¹⁴ are not replaced.

In some embodiments, the amino acid residues at positions Z⁶, Z⁷, Z¹¹ and Z¹⁴ are not replaced.

In some embodiments, the amino acid residues at positions Z⁶, Z¹¹, Z¹² and Z¹⁴, when an amino acid residue is present at the position, are not replaced.

In some embodiments, the amino acid residues at positions Z⁶, Z⁷, Z¹¹, Z¹² and Z¹⁴, when an amino acid residue is present at the position, are not replaced.

In some embodiments, the amino acid residues at positions Z³, Z⁶, Z⁷, Z¹¹, Z¹² and Z¹⁴, when an amino acid residue is present at the position, are not replaced.

In some embodiments, the amino acid residues at positions Z⁶, Z⁷, Z¹¹, Z¹², Z¹⁴ and Z¹⁵, when an amino acid residue is present at the position, are not replaced.

In some embodiments, the amino acid residues at positions Z³, Z⁶, Z⁷, Z¹¹, Z¹², Z¹⁴ and Z¹⁵, when an amino acid residue is present at the position, are not replaced.

In some embodiments, the amino acid residues at positions Z⁶, Z⁷, Z¹¹, Z¹², Z¹⁴ and Z¹⁶, when an amino acid residue is present at the position, are not replaced.

In some embodiments, the amino acid residues at positions Z³, Z⁶, Z⁷, Z¹¹, Z¹², Z¹⁴ and Z¹⁶, when an amino acid residue is present at the position, are not replaced.

In some embodiments, the amino acid residues at positions Z⁶, Z⁷, Z¹¹, Z¹², Z¹⁴, Z¹⁵ and Z¹⁶, when an amino acid residue is present at the position, are not replaced.

In some embodiments, the amino acid residues at positions Z³, Z⁶, Z⁷, Z¹¹, Z¹², Z¹⁴, Z¹⁵ and Z¹⁶, when an amino acid residue is present at the position, are not replaced.

In some embodiments, three or fewer of the amino acid residues at positions Z³, Z¹², Z¹⁵ and Z¹⁶ are absent.

In some embodiments, three of the amino acid residues at positions Z³, Z¹², Z¹⁵ and Z¹⁶ are absent.

In some embodiments, two or fewer of the amino acid residues at positions Z³, Z¹², Z¹⁵ and Z¹⁶ are absent.

In some embodiments, two of the amino acid residues at positions Z³, Z¹², Z¹⁵ and Z¹⁶ are absent.

In some embodiments, one or fewer of the amino acid residues at positions Z³, Z¹², Z¹⁵ and Z¹⁶ are absent.

In some embodiments, one of the amino acid residues at positions Z³, Z¹², Z¹⁵ and Z¹⁶ is absent.

In some embodiments, none of the amino acid residues at positions Z³, Z¹², Z¹⁵ and Z¹⁶ are absent.

In some embodiments, the amino acid residue at position Z³ is absent and the amino acid residues at positions Z¹², Z¹⁵ and Z¹⁶ are present.

In some embodiments, the amino acid residue at position Z¹² is absent and the amino acid residues at positions Z³, Z¹⁵ and Z¹⁶ are present.

In some embodiments, the amino acid residue at position Z¹⁵ is absent and the amino acid residues at positions Z³, Z¹² and Z¹⁶ are present.

In some embodiments, the amino acid residue at position Z¹⁶ is absent and the amino acid residues at positions Z³, Z¹² and Z¹⁵ are present.

In some embodiments, the amino acid residues at positions Z³ and Z¹⁶ are absent and the amino acid residues at positions Z¹² and Z¹⁵ are present.

In some embodiments, the amino acid residues at positions Z³, Z¹⁵ and Z¹⁶ are absent and the amino acid residue at position Z¹² is present.

In some embodiments, at least one of R^{NT}, when present, and/or R^{CT}, when present, and/or the amino acid residues at positions Z³, Z⁵, Z⁶, Z⁷, Z⁸, Z°, Z¹¹, Z¹², Z¹³, Z¹⁴, Z¹⁵ and/or Z¹⁶, when an amino acid residue is present at the position, each independently comprise(s) at least one quaternary amine.

In some embodiments, at least one of R^{NT}, when present, and/or R^{CT}, when present, and/or the amino acid residues at positions Z³, Z⁵, Z⁶, Z⁷, Z⁸, Z°, Z¹¹, Z¹², Z¹³, Z¹⁴, Z¹⁵ and/or Z¹⁶, when an amino acid residue is present at the position, each independently comprise(s) a quaternary amine.

In some embodiments, at least one of the amino acid residues at positions Z³, Z⁵, Z⁶, Z⁷, Z⁸, Z¹⁰, Z¹¹, Z¹², Z¹³, Z¹⁴, Z¹⁵ and/or Z¹⁶, when an amino acid residue is present at the position, each independently comprise(s) at least one quaternary amine.

In some embodiments, at least one of the amino acid residues at positions Z³, Z⁵, Z⁶, Z⁷, Z⁸, Z¹⁰, Z¹¹, Z¹², Z¹³, Z¹⁴, Z¹⁵ and/or Z¹⁶, when an amino acid residue is present at the position, each independently comprise(s) a quaternary amine.

In some embodiments, at least one of R^{NT}, when present, and/or R^{CT}, when present, and/or the amino acid residues at positions Z³, Z⁵, Z⁸, Z¹⁰, Z¹² and/or Z¹³, when an amino acid residue is present at the position, each independently comprise(s) at least one quaternary amine.

In some embodiments, at least one of R^{NT}, when present, and/or R^{CT}, when present, and/or the amino acid residues at positions Z³, Z⁵, Z⁸, Z¹⁰, Z¹², and/or Z¹³, when an amino acid residue is present at the position, each independently comprise(s) a quaternary amine.

In some embodiments, at least one of R^{NT}, when present, and/or the amino acid residues at positions Z³, Z⁵, Z⁸, Z¹⁰, and/or Z¹³, when an amino acid residue is present at the position, each independently comprise(s) at least one quaternary amine.

In some embodiments, at least one of R^{NT}, when present, and/or the amino acid residues at positions Z³, Z⁵, Z⁸, Z¹⁰, and/or Z¹³, when an amino acid residue is present at the position, each independently comprise(s) a quaternary amine.

In some embodiments, R^{NT} and the amino acid residues at positions Z⁵ and Z¹⁰ each independently comprise at least one quaternary amine.

In some embodiments, R^{NT} and the amino acid residues at positions Z⁵ and Z¹⁰ each independently comprise a quaternary amine.

In some embodiments, R^{NT} and the amino acid residues at positions Z⁸ and Z¹⁰ each independently comprise at least one quaternary amine.

In some embodiments, R^{NT} and the amino acid residues at positions Z⁸ and Z¹⁰ each independently comprise a quaternary amine.

In some embodiments, R^{NT} and the amino acid residues at positions Z⁸ and Z¹³ each independently comprise at least one quaternary amine.

In some embodiments, R^{NT} and the amino acid residues at positions Z⁸ and Z¹³ each independently comprise a quaternary amine.

In some embodiments, the amino acid residues at positions Z⁸, Z¹⁰ and Z¹³ each independently comprise at least one quaternary amine.

In some embodiments, the amino acid residues at positions Z⁸, Z¹⁰ and Z¹³ each independently comprise a quaternary amine.

In some embodiments, R^{NT} and the amino acid residue at position Z³ each independently comprise at least one quaternary amine.

In some embodiments, R^{NT} and the amino acid residue at position Z³ each independently comprise a quaternary amine.

In some embodiments, R^{NT} and R^{CT} each independently comprise at least one quaternary amine.

In some embodiments, R^{NT} and R^{CT} each comprise a quaternary amine.

In some embodiments, R^{NT} and the amino acid residue at position Z⁸ each independently comprise at least one quaternary amine.

In some embodiments, R^{NT} and the amino acid residue at position Z⁸ each independently comprise a quaternary amine.

In some embodiments, R^{NT} and the amino acid residue at position Z¹⁰ each independently comprise at least one quaternary amine.

In some embodiments, R^{NT} and the amino acid residue at position Z¹⁰ each independently comprise a quaternary amine.

In some embodiments, the amino acid residues at positions Z³ and Z⁸ each independently comprise at least one quaternary amine.

In some embodiments, the amino acid residues at positions Z³ and Z⁸ each independently comprise a quaternary amine.

In some embodiments, the amino acid residues at positions Z³ and Z¹⁰ each independently comprise at least one quaternary amine.

In some embodiments, the amino acid residues at positions Z³ and Z¹⁰ each independently comprise a quaternary amine.

In some embodiments, the amino acid residues at positions Z⁵ and Z¹⁰ each independently comprise at least one quaternary amine.

In some embodiments, the amino acid residues at positions Z⁵ and Z¹⁰ each independently comprise a quaternary amine.

In some embodiments, the amino acid residues at positions Z⁸ and Z¹⁰ each independently comprise at least one quaternary amine.

In some embodiments, the amino acid residues at positions Z⁸ and Z¹⁰ each independently comprise a quaternary amine.

In some embodiments, the amino acid residues at positions Z⁸ and Z¹³ each independently comprise at least one quaternary amine.

In some embodiments, the amino acid residues at positions Z⁸ and Z¹³ each independently comprise a quaternary amine.

In some embodiments, the amino acid residues at positions Z¹⁰ and Z¹³ each independently comprise at least one quaternary amine.

In some embodiments, the amino acid residues at positions Z¹⁰ and Z¹³ each independently comprise a quaternary amine.

In some embodiments, R^{NT} comprises at least one quaternary amine.

In some embodiments, R^{NT} comprises a quaternary amine.

In some embodiments, R^{CT} comprises at least one quaternary amine.

In some embodiments, R^{CT} comprises a quaternary amine.

In some embodiments, the amino acid residue at position Z³ comprises at least one quaternary amine.

In some embodiments, the amino acid residue at position Z³ comprises a quaternary amine.

In some embodiments, the amino acid residue at position Z⁵ comprises at least one quaternary amine.

In some embodiments, the amino acid residue at position Z⁵ comprises a quaternary amine.

In some embodiments, the amino acid residue at position Z⁸ comprises at least one quaternary amine.

In some embodiments, the amino acid residue at position Z⁸ comprises a quaternary amine.

In some embodiments, the amino acid residue at position Z¹⁰ comprises at least one quaternary amine.

In some embodiments, the amino acid residue at position Z¹⁰ comprises a quaternary amine.

In some embodiments, the amino acid residue at position Z¹² comprises at least one quaternary amine.

In some embodiments, the amino acid residue at position Z¹² comprises a quaternary amine.

In some embodiments, the amino acid residue at position Z¹³ comprises at least one quaternary amine.

In some embodiments, the amino acid residue at position Z¹³ comprises a quaternary amine.

In some embodiments, the at least one quaternary amine is one or two quaternary amines.

In some embodiments, each quaternary amine is independently selected from the group consisting of:
(a)

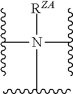

wherein each R^{ZA} is independently selected from the group consisting of: optionally substituted (C₁-C₂₀) alkyl and optionally substituted (C₁-C₂₀) heteroalkyl;
(b)

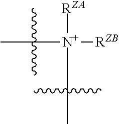

wherein each R^{ZA} and R^{ZB} is independently selected from the group consisting of: optionally substituted (C₁-C₂₀) alkyl and optionally substituted (C₁-C₂₀) heteroalkyl;
(c)

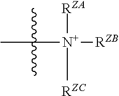

wherein:
(c)(i) each R^{ZA}, R^{ZB} and R^{ZC} is independently selected from the group consisting of: optionally substituted (C₁-C₂₀) alkyl and optionally substituted (C₁-C₂₀) heteroalkyl,
(c)(ii) each R^{ZA} is independently selected from the group consisting of: optionally substituted (C₁-C₂₀) alkyl and optionally substituted (C₁-C₂₀) heteroalkyl, and each R^{ZB} and R^{ZC} come together with the N atom to which they are attached to form an optionally substituted (C₃-C₁₄) heterocyclic ring, or
(c)(iii) each R^{ZA}, R^{ZB} and R^{ZC} come together with the N atom to which they are attached to form an optionally substituted (C₅-C₁₀) bicyclic heterocyclic ring;
(d)

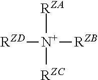

wherein:
(d)(i) each R^{ZA} and R^{ZB} is independently selected from the group consisting of: optionally substituted (C₁-C₂₀) alkyl and optionally substituted (C₁-C₂₀) heteroalkyl, and each R^{ZC} and R^{ZD} come together with the N atom to which they are attached to form an optionally substituted (C₃-C₁₄) heterocyclyl, or
(d)(ii) each R^{ZA} and R^{ZB} come together with the N atom to which they are attached to form an optionally substituted (C₃-C₁₄) heterocyclyl, and each R^{ZC} and R^{ZD} come together with the N atom to which they are attached to form an optionally substituted (C₃-C₁₄) heterocyclyl, or
(d)(iii) each R^{ZA} and R^{ZB} come together with the N atom to which they are attached to form an optionally substituted (C₃-C₁₄) heterocyclyl, and each R^{ZC} and R^{ZD} come together with the N atom to which they are attached to form an optionally substituted (C₃-C₁₄) heterocyclic ring;
(e)

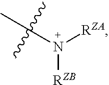

wherein each R^{ZA} and R^{ZB} come together with the N atom to which they are attached to form an optionally substituted (C₅-C₁₄) heteroaromatic ring, and
(f)

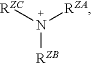

wherein each R^{ZA} is independently selected from the group consisting of: optionally substituted (C₁-C₂₀) alkyl and optionally substituted (C₁-C₂₀) heteroalkyl, and each R^{ZB} and R^{ZC} come together with the N atom to which they are attached to form an optionally substituted (C₅-C₁₄) heteroaryl.

In some embodiments, each quaternary amine is independently selected from the group consisting of:
(a)

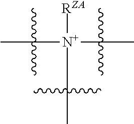

wherein each R^{ZA} is independently selected from the group consisting of: optionally substituted (C₁-C₁₆) alkyl and optionally substituted (C₁-C₈) heteroalkyl;
(b)

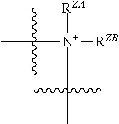

wherein each R^{ZA} and R^{ZB} is independently selected from the group consisting of: optionally substituted (C₁-C₁₆) alkyl and optionally substituted (C₁-C₈) heteroalkyl;
(c)

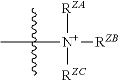

wherein:
(c)(i) each R^{ZA}, R^{ZB} and R^{ZC} is independently selected from the group consisting of: optionally substituted (C₁-C₁₆) alkyl and optionally substituted (C₁-C₈) heteroalkyl,
(c)(ii) each R^{ZA} is independently selected from the group consisting of: optionally substituted (C₁-C₁₆) alkyl and optionally substituted (C₁-C₈) heteroalkyl, and each R^{ZB} and R^{ZC} come together with the N atom to which they are attached to form an optionally substituted (C₆) heterocyclic ring, or
(c)(iii) each R^{ZA}, R^{ZB} and R^{ZC} come together with the N atom to which they are attached to form an optionally substituted (C₈) bicyclic heterocyclic ring;
(d)

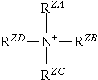

wherein:
(d)(i) each R^{ZA} and R^{ZB} is independently selected from the group consisting of: optionally substituted (C₁-C₁₆) alkyl and optionally substituted (C₁-C₈) heteroalkyl, and each R^{ZC} and R^{ZD} come together with the N atom to which they are attached to form an optionally substituted (C₆) heterocyclyl, or
(d)(ii) each R^{ZA} and R^{ZB} come together with the N atom to which they are attached to form an optionally substituted (C₆) heterocyclyl, and each R^{ZC} and R^{ZD} come together with the N atom to which they are attached to form an optionally substituted (C₆) heterocyclyl, or
(d)(iii) each R^{ZA} and R^{ZB} come together with the N atom to which they are attached to form an optionally substituted (C₆) heterocyclyl, and each R^{ZC} and R^{ZD} come together with the N atom to which they are attached to form an optionally substituted (C₆) heterocyclic ring;
(e)

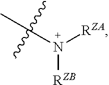

wherein each R^{ZA} and R^{ZB} come together with the N atom to which they are attached to form an optionally substituted (C₆) heteroaromatic ring; and
(f)

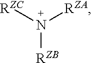

wherein each R^{ZA} is independently selected from the group consisting of: optionally substituted (C₁-C₁₆) alkyl and optionally substituted (C₁-C₈) heteroalkyl, and each R^{ZB} and R^{ZC} come together with the N atom to which they are attached to form an optionally substituted (C₆) heteroaryl.

In some embodiments, each quaternary amine is independently selected from the group consisting of:
(a)

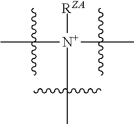

wherein each R^{ZA} is independently selected from the group consisting of: optionally substituted (C₁-C₁₆) alkyl and optionally substituted (C₁-C₈) heteroalkyl;
(b)

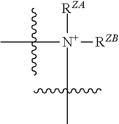

wherein each R^{ZA} and R^{ZB} is independently selected from the group consisting of: optionally substituted (C₁-C₁₆) alkyl and optionally substituted (C₁-C₈) heteroalkyl;
(c)

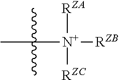

wherein:
(c)(i) each R^{ZA}, R^{ZB} and R^{ZC} is independently selected from the group consisting of: optionally substituted (C₁-C₁₆) alkyl and optionally substituted (C₁-C₈) heteroalkyl,
(c)(ii) each R^{ZA} is independently selected from the group consisting of: optionally substituted (C₁-C₃) alkyl and optionally substituted (C₁-C₃) heteroalkyl, and each R^{ZB} and R^{ZC} come together with the N atom to which they are attached to form an optionally substituted (C₆) heterocyclic ring, or
(c)(iii) each R^{ZA}, R^{ZB} and R^{ZC} come together with the N atom to which they are attached to form an optionally substituted (C₈) bicyclic heterocyclic ring;
(d)

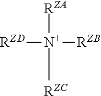

wherein:
(d)(i) each R^{ZA} and R^{ZB} is independently selected from the group consisting of: optionally substituted (C₁-C₃) alkyl and optionally substituted (C₁-C₃) heteroalkyl, and each R^{ZC} and R^{ZD} come together with the N atom to which they are attached to form an optionally substituted (C₆) heterocyclyl, or
(d)(ii) each R^{ZA} and R^{ZB} come together with the N atom to which they are attached to form an optionally substituted (C₆) heterocyclyl, and each R^{ZC} and R^{ZD} come together with the N atom to which they are attached to form an optionally substituted (C₆) heterocyclyl, or
(d)(iii) each R^{ZA} and R^{ZB} come together with the N atom to which they are attached to form an optionally substituted (C₆) heterocyclyl, and each R^{ZC} and R^{ZD} come together with the N atom to which they are attached to form an optionally substituted (C₆) heterocyclic ring;
(e)

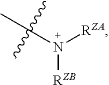

wherein each R^{ZA} and R^{ZB} come together with the N atom to which they are attached to form an optionally substituted (C₆) heteroaromatic ring; and
(f)

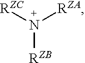

wherein each R^{ZA} is independently selected from the group consisting of: optionally substituted (C₁-C₃) alkyl and optionally substituted (C₁-C₃) heteroalkyl, and each R^{ZB} and R^{ZC} come together with the N atom to which they are attached to form an optionally substituted (C) heteroaryl.

In some embodiments, when each R^{ZA}, R^{ZB} and/or R^{ZC}, when present, is/are an optionally substituted alkyl, each optionally substituted alkyl is unsubstituted.

In some embodiments, when each R^{ZA}, R^{ZB} and/or R^{ZC}, when present, is/are an optionally substituted heteroalkyl, each optionally substituted heteroalkyl is unsubstituted.

In some embodiments, when each R^{ZB} and R^{ZC} come together with the N atom to which they are attached to form an optionally substituted heterocyclic ring, the optionally substituted heterocyclic ring is unsubstituted or substituted with one or more substitutents independently selected from the group consisting of Fluoro and (C₁-C₃) alkyl.

In some embodiments, when each R^{ZA}, R^{ZB} and R^{ZC} come together with the N atom to which they are attached to form an optionally substituted bicyclic heterocyclic ring, the optionally substituted bicyclic heterocyclic ring is unsubstituted.

In some embodiments, when each R^{ZA} and R^{ZB} come together with the N atom to which they are attached to form an optionally substituted heterocyclyl, the optionally substituted heterocyclyl is unsubstituted.

In some embodiments, when each R^{ZB} and R^{ZC} come together with the N atom to which they are attached to form an optionally substituted heteroaryl, the optionally substituted heteroaryl is unsubstituted.

In some embodiments, when each R^{ZC} and R^{ZD} come together with the N atom to which they are attached to form an optionally substituted heterocyclyl, the optionally substituted heterocyclyl is unsubstituted.

In some embodiments, when each R^{ZC} and R^{ZD} come together with the N atom to which they are attached to form an optionally substituted heterocyclic ring, the optionally substituted heterocyclic ring is unsubstituted.

In some embodiments, when each R^{ZA} and R^{ZB} come together with the N atom to which they are attached to form an optionally substituted heteroaromatic ring, the optionally substituted heteroaromatic ring is unsubstituted.

In some embodiments, each quaternary amine is independently selected from the group consisting of:

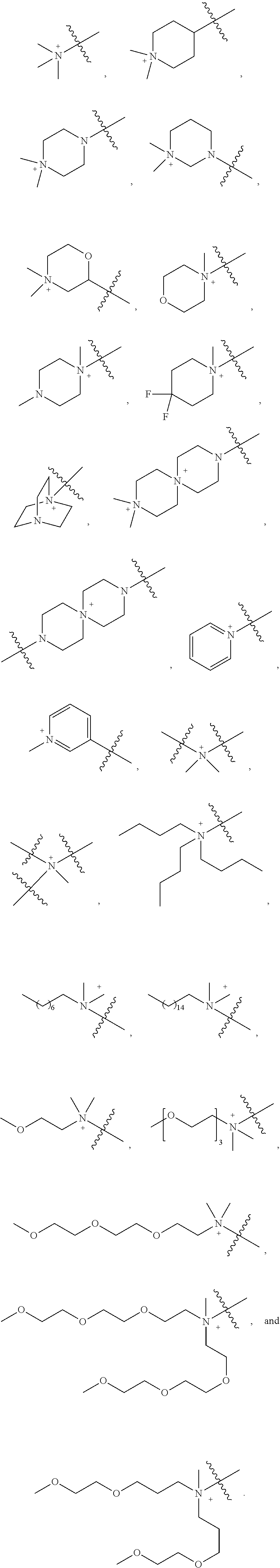

In some embodiments, each quaternary amine has an AN⁻counterion that is the same for each quaternary amine and is selected from the group consisting of: acetate, adipate, benzoate, benzenesulfonate, citrate, decanoate, chloride, lactate, maleate, methanesulfonate, oxalate, pivalate, propionate, succinate, sulfate, tartrate, or trifluoroacetate.

In some embodiments, at least one of the amino acid residues at positions Z³, Z⁵, Z⁶, Z⁷, Z⁸, Z¹⁰, Z¹¹, Z¹², Z¹³, Z¹⁴, Z¹⁵ and/or Z¹⁶, when an amino acid residue is present at the position, each independently comprise(s) at least one masked amine and/or masked amide.

In some embodiments, at least one of the amino acid residues at positions Z³, Z⁵, Z⁶, Z⁷, Z⁸, Z¹⁰, Z¹¹, Z¹², Z¹³, Z¹⁴, Z¹⁵ and/or Z¹⁶, when an amino acid residue is present at the position, each independently comprise(s) a masked amine and/or masked amide.

In some embodiments, at least one of the amino acid residues at positions Z⁵, Z⁷, Z⁸, Z¹⁰, Z¹³, Z¹⁴, Z¹⁵ and/or Z¹⁶, when an amino acid residue is present at the position, each independently comprise(s) at least one masked amine and/or masked amide.

In some embodiments, at least one of the amino acid residues at positions Z⁵, Z⁷, Z⁸, Z¹⁰, Z¹³, Z¹⁴, Z¹⁵ and/or Z¹⁶, when an amino acid residue is present at the position, each independently comprise(s) a masked amine and/or masked amide.

In some embodiments, at least one of the amino acid residues at positions Z⁵, Z⁸, and/or Z¹³, each independently comprise(s) at least one masked amine and/or masked amide.

In some embodiments, at least one of the amino acid residues at positions Z⁵, Z⁸, and/or Z¹³, each independently comprise(s) a masked amine and/or masked amide.

In some embodiments, the amino acid residue at position Z⁵ comprises at least one masked amine and/or masked amide.

In some embodiments, the amino acid residue at position Z⁵ comprises a masked amine and/or masked amide.

In some embodiments, the amino acid residue at position Z⁸ comprises at least one masked amine and/or masked amide.

In some embodiments, the amino acid residue at position Z⁸ comprises a masked amine and/or masked amide.

In some embodiments, the amino acid residue at position Z¹³ comprises at least one masked amine and/or masked amide.

In some embodiments, the amino acid residue at position Z¹³ comprises a masked amine and/or masked amide.

In some embodiments, at least one of the masked amine(s) is an amine in a backbone amide, wherein each amine in a backbone amide is independently substituted with (C₁-C₃) alkyl.

In some embodiments, at least one of the masked amine(s) and/or masked amide(s) is present in the side chain of the amino acid residue comprising said masked amine and/or masked amide(s).

In some embodiments, at least one of the masked amine(s) and/or masked amide(s) is independently selected from the group consisting of:
(a)

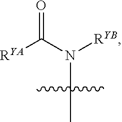

wherein each R^{YA} is independently selected from the group consisting of: optionally substituted (C₁-C₁₀) alkyl and optionally substituted (C₁-C₂₀) heteroalkyl, and each R^{YB} is independently selected from the group consisting of: hydrogen, optionally substituted (C₁-C₁₀) alkyl and optionally substituted (C₁-C₂₀) heteroalkyl;
(b)

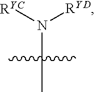

wherein each R^{YC} is independently selected from the group consisting of: optionally substituted (C₁-C₁₀) alkyl and optionally substituted (C₁-C₂₀) heteroalkyl, and each R^{YD} is independently selected from the group consisting of: hydrogen, optionally substituted (C₁-C₁₀) alkyl and optionally substituted (C₁-C₂₀) heteroalkyl;
(c)

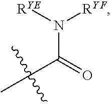

wherein each R^{YE} is independently selected from the group consisting of: optionally substituted (C₁-C₁₀) alkyl and optionally substituted (C₁-C₂₀) heteroalkyl, and each R^{YF} is independently selected from the group consisting of: hydrogen, optionally substituted (C₁-C₁₀) alkyl and optionally substituted (C₁-C₂₀) heteroalkyl; and
(d)

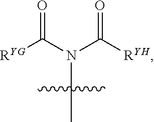

wherein each R^{YG} and R^{YH} is independently selected from the group consisting of: optionally substituted (C₁-C₁₀) alkyl and optionally substituted (C₁-C₂₀) heteroalkyl.

In some embodiments, at least one of the masked amine(s) and/or masked amides is independently selected from the group consisting of:
(a)

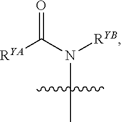

wherein each R^{YA} is independently selected from the group consisting of: optionally substituted (C₁-C₅) alkyl and optionally substituted (C₁-C₁₅) heteroalkyl, and each R^{YB} is independently selected from the group consisting of hydrogen, optionally substituted (C₁-C₅) alkyl and optionally substituted (C₁-C₁₅) heteroalkyl;
(b)

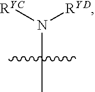

wherein each R^{YC} is independently selected from the group consisting of optionally substituted (C₁-C₅) alkyl and optionally substituted (C₁-C₁₅) heteroalkyl, and each R^{YD} is independently selected from the group consisting of hydrogen, optionally substituted (C₁-C₅) alkyl and optionally substituted (C₁-C₁₅) heteroalkyl;
(c)

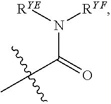

wherein each R^{YE} is independently selected from the group consisting of optionally substituted (C₁-C₅) alkyl and optionally substituted (C₁-C₁₅) heteroalkyl, and each R^{YF} is independently selected from the group consisting of hydrogen, optionally substituted (C₁-C₅) alkyl and optionally substituted (C₁-C₁₅) heteroalkyl; and
(d)

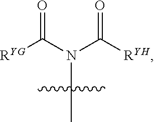

wherein each R^{YG} and R^{YH} is independently selected from the group consisting of optionally substituted (C₁-C₅) alkyl and optionally substituted (C₁-C₁₅) heteroalkyl.

In some embodiments, when each R^{YA}, R^{YB}, R^{YC}, R^{YD}, R^{YE}, R^{YF}, R^{YG} and/or R^{YH} is optionally substituted alkyl, each optionally substituted alkyl is independently unsubstituted or substituted with one or more substitutents independently selected from the group consisting of —CO₂H and a quaternary amine as described herein, for example, —N⁺(CH₃)₃.

In some embodiments, when each R^{YA}, R^{YB}, R^{YC}, R^{YD}, R^{YE}, R^{YF}, R^{YG} and/or R^{YH} is optionally substituted heteroalkyl, each optionally substituted heteroalkyl is independently unsubstituted or substituted with one or more substitutents independently selected from the group consisting of: —CO₂H and a quaternary amine as described herein, for example, —N⁺(CH₃)₃.

In some embodiments, at least one of the masked amine(s) and/or masked amide(s) is independently selected from the group consisting of:

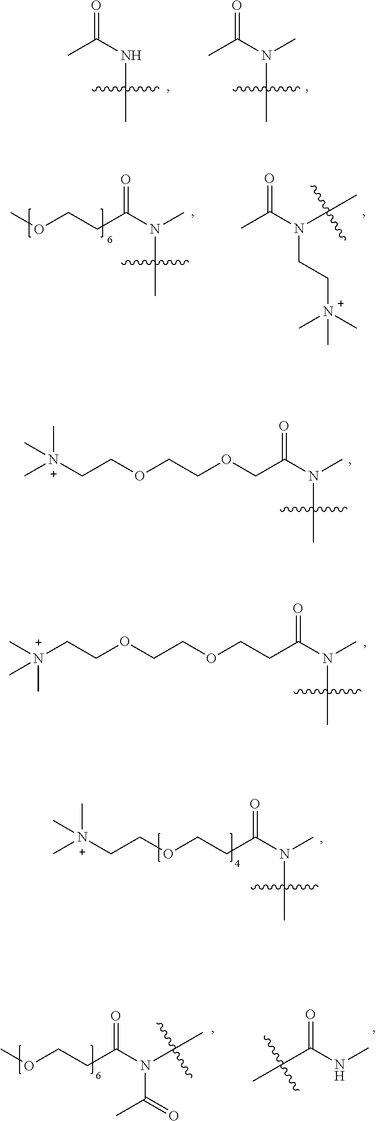

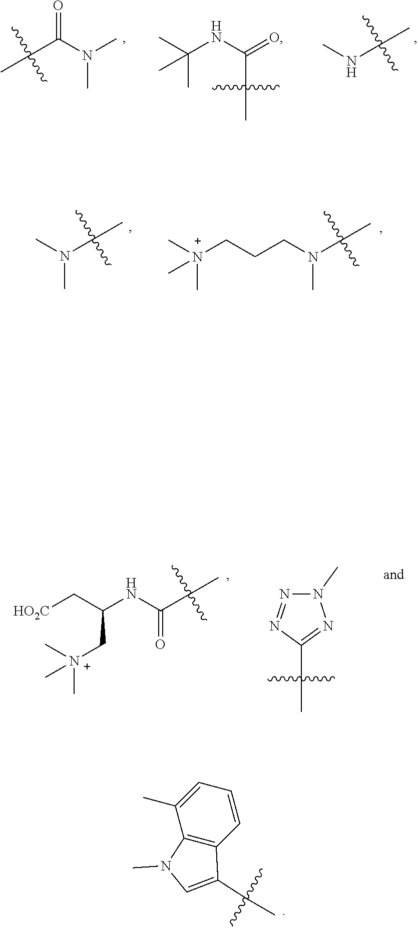

In some embodiments, the at least one amino acid residue(s) each independently comprising at least one masked amine and/or masked amide is/are independently selected from the group consisting of: NMe7MeW, AAMPhe, Paf(Ac), AEF(Ac), AcAEF, AEF(AcCh), AEF(Me)2, AEF(N(Me)2), AEF(MePrpa), AEF(NMe), Dab(NMeAc), Dab(NMecarn), Dab(NMeCOmPEG6), Dab(NMecPEG2a), Dab(NMecPEG3a), Dab(NMecPEG5a), Dab(NMecPEG5aCO)-, Dap(Ac), Dap(NMeAc), K(Ac), K(NMeAc), K(NMeCOmPEG6), K(NMeCOPEG4N+Me3), K(NMeCOPEG5a), K(NMecPEG5a), K(NMePEG5a), K(NMePEG3a), K(NmPEG6Ac), NMe3Pya, NMebAla, NMeDTyr, N(N(Me)), N(NMe), N(N(Me)2), Q(N(Me)2), Q(NHtBu) and Tetrazole(NMe).

In some embodiments, the at least one amino acid residue(s) each independently comprising at least one masked amine is not K(Ac).

In some embodiments, when the amino acid residue at position Z³ is replaced with an amino acid residue that is different from the amino acid residue recited at said position, the amino acid residue at position Z³ is replaced with an amino acid residue selected from the group consisting of: APEG2ser, APEG2Ser, APEG2Ser(S*), e(c), e(C), hk(Me)₃, k(5cpa), k(cPEG3a), k(d), k(D), k(dPEG12Ac), k(dPEG6Ac), k(dPEG9Ac), k(Me)₃, K(Me)₃, k(PEG2PEG2gEC12), k(PEG2PEG2gEC14), k(PEG2PEG2PEG2PEG2gEC12), k(PEG2PEG6gEC12), SP6, APEG2Ser(RS), gPEG2Ser and k(PEG2PEG2gE(c)C12.

In some embodiments, when the amino acid residue at position Z³ is replaced with an amino acid residue that is different from the amino acid residue recited at said position, the amino acid residue at position Z³ is replaced with an amino acid residue comprising at least one quaternary amine selected from the group consisting of: APEG2ser, APEG2Ser, e(c), e(C), hk(Me)₃, k(5cpa), k(cPEG3a), k(d), k(D), K(Me)₃ and k(Me)₃.

In some embodiments, when the amino acid residue at position Z³ is replaced with an amino acid residue that is different from the amino acid residue recited at said position, the amino acid residue at position Z³ is replaced with an amino acid residue that is r substituted on the side chain with one or more groups selected from: —OH, —(C₁-C₄) alkyl, —O(C₁-C₄) alkyl, and —CN.

In some embodiments, when the amino acid residue at position Z³ is replaced with an amino acid residue that is different from the amino acid residue recited at said position, the amino acid residue at position Z³ is replaced with a hydrophilic amino acid residue.

In some embodiments, when the amino acid residue at position Z³ is replaced with an amino acid residue that is different from the amino acid residue recited at said position, the amino acid residue at position Z³ is replaced with a basic amino acid residue.

In some embodiments, when the amino acid residue at position Z³ is replaced with an amino acid residue that is different from the amino acid residue recited at said position, the amino acid residue at position Z³ is replaced with an acidic amino acid residue.

In some embodiments, the amino acid residue at position Z³ is replaced with an amino acid residue that is different from the amino acid residue recited at said position.

In some embodiments, the amino acid residue at position Z⁴ is the residue of Abu or the residue of an amino acid comprising a sulfhydryl group selected from the group consisting of: Pen, C or aMeC.

In some embodiments, the amino acid residue at position Z⁴ is the residue of Pen.

In some embodiments, when the amino acid residue at position Z⁵ is replaced with an amino acid residue that is different from the amino acid residue recited at said position, the amino acid residue at position Z⁵ is replaced with an amino acid residue selected from the group consisting of: A, APEG2Ser(S*), Dab(Me)₃, F, Gab, K(cPEG3a), K(Me)₃, K(PEG2PEG2gEC12), K(PEG2PEG2gEC14), L, N, N(N(Me)), N(NMe), Q and W.

In some embodiments, when the amino acid residue at position Z⁵ is replaced with an amino acid residue that is different from the amino acid residue recited at said position, the amino acid residue at position Z⁵ is replaced with an amino acid residue comprising at least one quaternary amine selected from the group consisting of: APEG2Ser(S*), Dab(Me)₃, K(cPEG3a) and K(Me)₃.

In some embodiments, when the amino acid residue at position Z⁵ is replaced with an amino acid residue that is different from the amino acid residue recited at said position, the amino acid residue at position Z⁵ is replaced with an amino acid residue that is N(N(Me)₂) substituted on the side chain with one or more groups selected from: —OH, —(C₁-C₄) alkyl, —O(C₁-C₄) alkyl, and —CN.

In some embodiments, when the amino acid residue at position Z⁵ is replaced with an amino acid residue that is different from the amino acid residue recited at said position, the amino acid residue at position Z⁵ is replaced with an aromatic amino acid residue.

In some embodiments, when the amino acid residue at position Z⁵ is replaced with an amino acid residue that is different from the amino acid residue recited at said position, the amino acid residue at position Z⁵ is replaced with an aliphatic amino acid residue.

In some embodiments, when the amino acid residue at position Z⁵ is replaced with an amino acid residue that is different from the amino acid residue recited at said position, the amino acid residue at position Z⁵ is replaced with a hydrophilic amino acid residue.

In some embodiments, when the amino acid residue at position Z⁵ is replaced with an amino acid residue that is different from the amino acid residue recited at said position, the amino acid residue at position Z⁵ is replaced with a basic amino acid residue.

In some embodiments, when the amino acid residue at position Z⁵ is replaced with an amino acid residue that is different from the amino acid residue recited at said position, the amino acid residue at position Z⁵ is replaced with an acidic amino acid residue.

In some embodiments, the amino acid residue at position Z⁵ is replaced with an amino acid residue that is different from the amino acid residue recited at said position.

In some embodiments, when the amino acid residue at position Z⁶ is replaced with an amino acid residue that is different from the amino acid residue recited at said position, the amino acid residue at position Z⁶ is replaced with an amino acid residue selected from the group consisting of: A and L.

In some embodiments, when the amino acid residue at position Z⁶ is replaced with an amino acid residue that is different from the amino acid residue recited at said position, the amino acid residue at position Z⁶ is replaced with an amino acid residue that is T substituted on the side chain with one or more groups selected from: —OH, —(C₁-C₄) alkyl, —O(C₁-C₄) alkyl, and —CN.

In some embodiments, when the amino acid residue at position Z⁶ is replaced with an amino acid residue that is different from the amino acid residue recited at said position, the amino acid residue at position Z⁶ is replaced with an aliphatic amino acid residue.

In some embodiments, when the amino acid residue at position Z⁶ is replaced with an amino acid residue that is different from the amino acid residue recited at said position, the amino acid residue at position Z⁶ is replaced with a polar amino acid residue.

In some embodiments, the amino acid residue at position Z⁶ is replaced with an amino acid residue that is different from the amino acid residue recited at said position.

In some embodiments, when the amino acid residue at position Z⁷ is replaced with an amino acid residue that is different from the amino acid residue recited at said position, the amino acid residue at position Z⁷ is replaced with an amino acid residue selected from the group consisting of: W, 7(3NAcPh)W, 7CF3W, NMe7MeW, 2Nal, A, F and L.

In some embodiments, when the amino acid residue at position Z⁷ is replaced with an amino acid residue that is different from the amino acid residue recited at said position, the amino acid residue at position Z⁷ is replaced with an amino acid residue that is 7MeW substituted on the side chain with one or more groups selected from: —OH, —(C₁-C₄) alkyl, —O(C₁-C₄) alkyl, and —CN.

In some embodiments, when the amino acid residue at position Z⁷ is replaced with an amino acid residue that is different from the amino acid residue recited at said position, the amino acid residue at position Z⁷ is replaced with an aromatic amino acid residue.

In some embodiments, when the amino acid residue at position Z⁷ is replaced with an amino acid residue that is different from the amino acid residue recited at said position, the amino acid residue at position Z⁷ is replaced with an aliphatic amino acid residue.

In some embodiments, the amino acid residue at position Z⁷ is replaced with an amino acid residue that is different from the amino acid residue recited at said position.

In some embodiments, when the amino acid residue at position Z⁸ is replaced with an amino acid residue that is different from the amino acid residue recited at said position, the amino acid residue at position Z⁸ is replaced with an amino acid residue selected from the group consisting of: K(NMeAC), K(NMeAc), Q, 4AmPhe, A, AIB, APEG2Ser, APEG2Ser(R*), APEG2Ser(S*), Cit, Dab(NMeAc), Dab(NMecarn), Dab(NMeCarn), Dab(NMeCOmPEG6), Dab(NMecPEG2a), Dab(NMecPEG3a), Dab(NMecPEG5a), Dap(NMeAc), F, K(4cpg), K(Ac), K(cPEG3a), K(Me)₃, K(NMeCOmPEG6), K(NMePEG3a), K(NmPEG6Ac), K(PEG2PEG2gEC12), K(PEG2PEG2gEC14), L, Paf(Ac), Q(N(Me)2), Q(NHtBu), W, Y and K(cPEG3aCO).

In some embodiments, when the amino acid residue at position Z⁸ is replaced with an amino acid residue that is different from the amino acid residue recited at said position, the amino acid residue at position Z⁸ is replaced with an amino acid residue comprising at least one quaternary amine selected from the group consisting of: APEG2Ser, APEG2ser, APEG2Ser(R*), APEG2Ser(S*), Dab(NMecarn), Dab(NMeCarn), Dab(NMecPEG2a), Dab(NMecPEG3a), Dab(NMecPEG5a), K(cPEG3a), K(Me)₃ and K(NMePEG3a).

In some embodiments, when the amino acid residue at position Z⁸ is replaced with an amino acid residue that is different from the amino acid residue recited at said position, the amino acid residue at position Z⁸ is replaced with an amino acid residue that is K(NMeAc) substituted on the side chain with one or more groups selected from: —OH, —(C₁-C₄) alkyl, —O(C₁-C₄) alkyl, and —CN.

In some embodiments, when the amino acid residue at position Z⁸ is replaced with an amino acid residue that is different from the amino acid residue recited at said position, the amino acid residue at position Z⁸ is replaced with an aromatic amino acid residue.

In some embodiments, when the amino acid residue at position Z⁸ is replaced with an amino acid residue that is different from the amino acid residue recited at said position, the amino acid residue at position Z⁸ is replaced with an aliphatic amino acid residue.

In some embodiments, when the amino acid residue at position Z⁸ is replaced with an amino acid residue that is different from the amino acid residue recited at said position, the amino acid residue at position Z⁸ is replaced with a hydrophilic amino acid residue.

In some embodiments, when the amino acid residue at position Z⁸ is replaced with an amino acid residue that is different from the amino acid residue recited at said position, the amino acid residue at position Z⁸ is replaced with a basic amino acid residue.

In some embodiments, when the amino acid residue at position Z⁸ is replaced with an amino acid residue that is different from the amino acid residue recited at said position, the amino acid residue at position Z⁸ is replaced with an acidic amino acid residue.

In some embodiments, the amino acid residue at position Z⁸ is replaced with an amino acid residue that is different from the amino acid residue recited at said position.

In some embodiments, the amino acid residue at position Z⁹ is the residue of an amino acid comprising a sulfhydryl group selected from the group consisting of: Pen, C or aMeC.

In some embodiments, the amino acid residue at position Z⁹ is the residue of Pen.

In some embodiments, when the amino acid residue at position Z¹⁰ is replaced with an amino acid residue that is different from the amino acid residue recited at said position, the amino acid residue at position Z¹⁰ is replaced with an amino acid residue selected from the group consisting of: AEF, 4DMPEF, 4DMPzEF, 4TMABYF, ACHMF, AEF((Ch)cPEG3a), AEF(Ac), AEF(AcCh), AEF(aPEG2a), AEF(BisMEP), AEF(BisMEPa), AEF(BisPEG2a)(RS), AEF(BisPEG2a)(S*), AEF(G), AEF(Me)2, AEF(MEP), AEF(MePrpa), AEF(N(Me)2), AEF(NHCh), AEF(NHcPEG3a), AEF(NMe), AEF(NMe2mPEG3), AEF(NMe3), AEF(NMeBismPEG3), AEF(NMePEG2a), AEF(NmPEG6), AEF(NsCh), AEF(PEG2a), AEF(SPD), APEG2F, APEG3F, dFPPEG3F, Diazabiclyclooctane6F, DMMMF, DMPMF, DMTASF, F(4G), F(4N3), F(4TzlDMA4mPEG), F(4TzlMME), F(4TzlMMol), F(4TzlMMo3), F(4TzlMMo4), F(4TzlTMA1), F(4TzlTMA2), F(4TzlTMA3), F(4TzlTMA4), F(4TzlTMA5), GPEG3F, hFTMAPF, MMoEF, MMoPF, MMPEG3F, morfTMA4F, mPEG2TMA2F, mPEG2TMA4F, mPEG3TMA4F, MPzPEG3F, MTASF, NPyEF, NPyPEG3F, PiperazinequatF, TBAPEG3F, TMA3F, TMA4F, TMA6F, TMA8F, Tzl(Ch), TzlChmPEG, TzlChmPEG3, Y(C₉₀H), Y(OEOXIMECh), Y(OTzlCh), Y(OTzlChC16), Y(OTzlChC8), Y(OTzlC1aC8), Y(OTzlChmPEG), Y(OTzlChmPEG3), Y(OTzlPEG3a), Y(OTzlPEG4a), Y(OTzlTMA4), Y(OZOXIMECh), YC8CO(NHPEG3a), YC8COPip, YCF2H, ACHMF(R*, S*), ACHMF(S*, S*), AEF(cPEG3a), APF, F(4TzlAme2), F(4TzlG2), F(4TzlMMo7) and F(4TzlTMA7).

In some embodiments, when the amino acid residue at position Z¹⁰ is replaced with an amino acid residue that is different from the amino acid residue recited at said position, the amino acid residue at position Z¹⁰ is replaced with an amino acid residue comprising at least one quaternary amine selected from the group consisting of: 4DMPEF, 4DMPzEF, 4TMABYF, ACHMF, ACHMF(R*, S*), ACHMF(S*, S*), AEF((Ch)cPEG3a), AEF(AcCh), AEF(aPEG2a), AEF(cPEG3a), AEF(BisMEPa), AEF(BisPEG2a)(RS), AEF(BisPEG2a)(S*), AEF(MePrpa), AEF(NHcPEG3a), AEF(NMe2mPEG3), AEF(NMe3), AEF(NMeBismPEG3), AEF(NMePEG2a), AEF(NsCh), AEF(NHCh), AEF(PEG2a), AEF(SPD), APEG2F, APEG3F, dFPPEG3F, Diazabiclyclooctane6F, DMMMF, DMPMF, DMTASF, F(4TzlDMA4mPEG), F(4TzlMMol), F(4TzlMMo3), F(4TzlMMo4), F(4TzlTMA1), F(4TzlTMA2), F(4TzlTMA3), F(4TzlTMA4), F(4TzlTMA5), F(4TzlMMo7), F(4TzlTMA7), hFTMAPF, MMoEF, MMoPF, MMPEG3F, morfTMA4F, mPEG2TMA2F, mPEG2TMA4F, mPEG3TMA4F, MPzPEG3F, MTASF, NPyEF, NPyPEG3F, PiperazinequatF, TBAPEG3F, TMA3F, TMA4F, TMA6F, TMA8F, Tzl(Ch), TzlChmPEG, TzlChmPEG3, Y(OEOXIMECh), Y(OTzlCh), Y(OTzlChC16), Y(OTzlChC8), Y(OTzlC1aC8), Y(OTzlChmPEG), Y(OTzlChmPEG3), Y(OTzlPEG3a), Y(OTzlPEG4a), Y(OTzlTMA4), Y(OZOXIMECh) and YC8CO(NHPEG3a).

In some embodiments, when the amino acid residue at position Z¹⁰ is replaced with an amino acid residue that is different from the amino acid residue recited at said position, the amino acid residue at position Z¹⁰ is replaced with an amino acid residue that is TMAPF substituted on the side chain with one or more groups selected from: —OH, —(C₁-C₄) alkyl, —O(C₁-C₄) alkyl, and —CN.

In some embodiments, when the amino acid residue at position Z¹⁰ is replaced with an amino acid residue that is different from the amino acid residue recited at said position, the amino acid residue at position Z¹⁰ is replaced with an amino acid residue having the following structure:

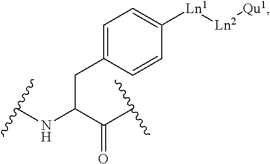

wherein:

Ln¹ is selected from the group consisting of: an —O— atom,

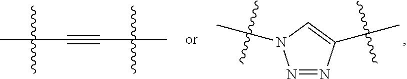

wherein the right hand side of each depicted structure is bound to Ln²;
Ln² is a linker moiety comprising:
(i) at least 4 atoms, for example an optionally substituted alkylene comprising at least C₄ atoms, an optionally substituted heteroalkylene comprising at least C₄ atoms, an optionally substituted heteroalkenylene comprising at least C₄ atoms, an optionally substituted alkynylene comprising at least C₄ atoms, an optionally substituted alkylene—optionally substituted carbocyclyl comprising at least C₄ atoms, or an optionally substituted alkylene—optionally substituted heterocyclyl comprising at least C₄ atoms, for example optionally substituted (C₄-C₂₀) alkylene, optionally substituted (C₄-C₂₀) heteroalkylene, optionally substituted (C₄-C₂₀) heteroalkenylene, optionally substituted (C₄-C₂₀) alkynylene, optionally substituted (C₁-C₃) alkylene —optionally substituted (C₃-C₁₀) carbocyclyl, or optionally substituted (C₁-C₃) alkylene—optionally substituted (C₃-C₁₄) heterocyclyl, or
(ii) at least 2 atoms when Ln¹ is

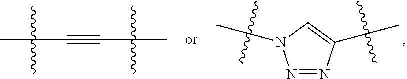

for example an optionally substituted alkylene comprising at least C₂ atoms or an optionally substituted heteroalkylene comprising at least C₂ atoms, for example optionally substituted (C₂-C₂₀) alkylene or optionally substituted (C₂-C₂₀) heteroalkylene; and
Qu¹ is a quaternary amine.

In some embodiments, when the amino acid residue at position Z¹⁰ is replaced with an amino acid residue that is different from the amino acid residue recited at said position, the amino acid residue at position Z¹⁰ is replaced with an amino acid residue having the following structure:

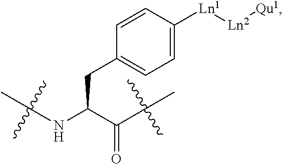

wherein Ln¹, Ln² and Qu¹ are as defined herein.

In some embodiments, Qu¹ is a quaternary amine as defined herein.

In some embodiments, Ln¹ is an —O— atom.

In some embodiments, Ln² is selected from the group consisting of: optionally substituted (C₄-C₁₅) alkylene, optionally substituted (C₄-C₁₅) heteroalkylene, optionally substituted (C₄-C₁₅) heteroalkenylene, optionally substituted (C₄-C₁₅) alkynylene, optionally substituted (C₁-C₃) alkylene—optionally substituted (C₅-C₇) carbocyclyl, or optionally substituted (C₁-C₃) alkylene—optionally substituted (C₅-C₇) heterocyclyl.

In some embodiments, Ln² is selected from the group consisting of: optionally substituted (C₄-C₁₀) alkylene, optionally substituted (C₄-C₁₀) heteroalkylene, optionally substituted (C₄-C₁₀) heteroalkenylene, optionally substituted (C₄-C₁₀) alkynylene, optionally substituted (C₁-C₃) alkylene—optionally substituted (C₆) carbocyclyl, or optionally substituted (C₁-C₃) alkylene—optionally substituted (C₆) heterocyclyl.

In some embodiments, Ln² is selected from the group consisting of: optionally substituted (C₄-C₈) alkylene, optionally substituted (C₄-C₈) heteroalkylene, optionally substituted (C₄-C₈) heteroalkenylene, optionally substituted (C₄-C₈) alkynylene, optionally substituted (C₁-C₃) alkylene—optionally substituted (C₆) carbocyclyl, or optionally substituted (C₁-C₃) alkylene—optionally substituted (C₆) heterocyclyl.

In some embodiments, when Ln¹ is

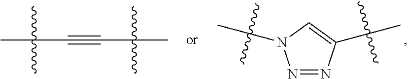

Ln² is selected from the group consisting of optionally substituted (C₂-C₁₅) alkylene or optionally substituted (C₂-C₁₅) heteroalkylene.

In some embodiments, when Ln¹ is

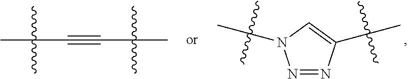

Ln² is selected from the group consisting of optionally substituted (C₂-C₁₀) alkylene or optionally substituted (C₂-C₁₀) heteroalkylene.

In some embodiments, when Ln¹ is

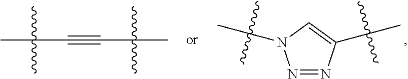

Ln² is selected from the group consisting of optionally substituted (C₂-C₅) alkylene or optionally substituted (C₂-C₅) heteroalkylene.

In some embodiments, when Ln² is optionally substituted alkylene, the optionally substituted alkylene is unsubstituted or is substituted with one or more substituents independently selected from the group consisting of Fluoro.

In some embodiments, when Ln² is optionally substituted heteroalkylene, the optionally substituted heteroalkylene is unsubstituted or is substituted with one or more substituents independently selected from the group consisting of: (C₁-C₃) alkyl,

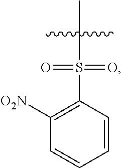 [SMILES: *S(=O)(=O)c1ccccc1[N+](=O)[O-]]

═O (i.e., two geminal hydrogens on a carbon atom of the heteroalkylene are replaced with the group ═O), or a quaternary amine described herein, for example, —N⁺(CH₃)₃.

In some embodiments, when Ln² is optionally substituted heteroalkenylene, the optionally substituted heteroalkenylene is unsubstituted.

In some embodiments, when Ln² is optionally substituted alkynylene, the optionally substituted alkynylene is unsubstituted.

In some embodiments, when Ln² is optionally substituted alkylene —optionally substituted carbocyclyl, the optionally substituted alkylene—optionally substituted carbocyclyl is unsubstituted.

In some embodiments, when Ln² is optionally substituted alkylene —optionally substituted heterocyclyl, the optionally substituted alkylene—optionally substituted heterocyclyl is unsubstituted.

In some embodiments, when the amino acid residue at position Z¹⁰ is replaced with an amino acid residue that is different from the amino acid residue recited at said position, the amino acid residue at position Z¹⁰ is replaced with an amino acid residue having the following structure:

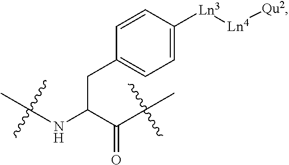

wherein:
Ln³ is selected from the group consisting of: an —O— atom,

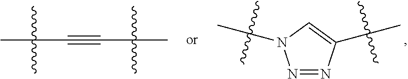

wherein the right hand side of each depicted structure is bound to Ln⁴;
Ln⁴ is a linker moiety comprising at least 2 atoms, for example an optionally substituted alkylene comprising at least C₂ atoms, an optionally substituted heteroalkylene comprising at least C₂ atoms, or an optionally substituted alkynylene comprising at least C₂ atoms, for example optionally substituted (C₂-C₂₀) alkylene, optionally substituted (C₂-C₂₀) heteroalkylene or optionally substituted (C₂-C₂₀) alkynylene; and Qu² is a (C₃-C₁₄) heterocyclyl quaternary amine, a (C₅-C₁₄) heteroaryl quaternary amine or a (C₅-C₁₄) bicyclic heterocyclyl quaternary amine, for example, a (C₃-C₁₄) heterocyclyl quaternary amine selected from the group consisting of:

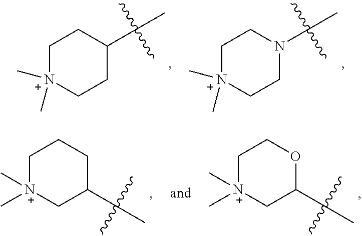

a (C₅-C₁₄) heteroaryl quaternary amine selected from the group consisting of:

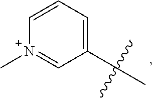 [SMILES: *c1ccc[n+](C)c1]

or a (C₅-C₁₄) bicyclic heterocyclyl quaternary amine selected from the group consisting of

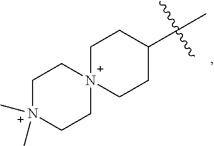 [SMILES: *C1CC[N+]2(CC1)CC[N+](C)(C)CC2]

In some embodiments, when the amino acid residue at position Z¹⁰ is replaced with an amino acid residue that is different from the amino acid residue recited at said position, the amino acid residue at position Z¹⁰ is replaced with an amino acid residue having the following structure:

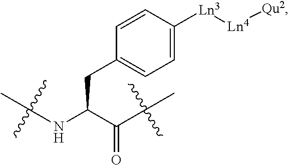

wherein Ln³, Ln⁴ and Qu² are as defined herein.

In some embodiments, Qu² is a quaternary amine as defined herein.

In some embodiments, Ln³ is an —O— atom.

In some embodiments, Ln⁴ is selected from the group consisting of: optionally substituted (C₂-C₁₅) alkylene, optionally substituted (C₂-C₁₅) heteroalkylene or optionally substituted (C₂-C₁₅) alkynylene.

In some embodiments, Ln⁴ is selected from the group consisting of: optionally substituted (C₂-C₁₀) alkylene, optionally substituted (C₂-C₁₀) heteroalkylene or optionally substituted (C₂-C₁₀) alkynylene.

In some embodiments, Ln⁴ is selected from the group consisting of: optionally substituted (C₂-C₈) alkylene, optionally substituted (C₂-C₈) heteroalkylene or optionally substituted (C₂-C₈) alkynylene.

In some embodiments, when Ln⁴ is optionally substituted alkylene, the optionally substituted alkylene is unsubstituted or is substituted with one or more substituents independently selected from the group consisting of Fluoro.

In some embodiments, when Ln⁴ is optionally substituted heteroalkylene, the optionally substituted heteroalkylene is unsubstituted or is substituted with one or more substituents independently selected from the group consisting of: (C₁-C₃) alkyl,

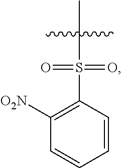 [SMILES: *S(=O)(=O)c1ccccc1[N+](=O)[O-]]

═O (i.e., two geminal hydrogens on a carbon atom of the heteroalkylene are replaced with the group ═O), or a quaternary amine as described herein, for example, —N⁺(CH₃)₃.

In some embodiments, when Ln⁴ is optionally substituted alkynylene, the optionally substituted alkynylene is unsubstituted.

In some embodiments, when the amino acid residue at position Z¹⁰ is replaced with an amino acid residue that is different from the amino acid residue recited at said position, the amino acid residue at position Z¹⁰ is replaced with an aromatic amino acid residue.

In some embodiments, the amino acid residue at position Z¹⁰ is replaced with an amino acid residue that is different from the amino acid residue recited at said position.

In some embodiments, when the amino acid residue at position Z¹¹ is replaced with an amino acid residue that is different from the amino acid residue recited at said position, the amino acid residue at position Z¹¹ is replaced with an amino acid residue selected from the group consisting of A, F, L and W.

In some embodiments, when the amino acid residue at position Z¹¹ is replaced with an amino acid residue that is different from the amino acid residue recited at said position, the amino acid residue at position Z¹¹ is replaced with an amino acid residue that is 2Nal substituted on the side chain naphthalene ring with one or more groups selected from: —OH, —(C₁-C₄) alkyl, —O(C₁-C₄) alkyl, and —CN.

In some embodiments, when the amino acid residue at position Z¹¹ is replaced with an amino acid residue that is different from the amino acid residue recited at said position, the amino acid residue at position Z¹¹ is replaced with an aromatic amino acid residue.

In some embodiments, when the amino acid residue at position Z¹¹ is replaced with an amino acid residue that is different from the amino acid residue recited at said position, the amino acid residue at position Z¹¹ is replaced with an aliphatic amino acid residue.

In some embodiments, the amino acid residue at position Z¹¹ is replaced with an amino acid residue that is different from the amino acid residue recited at said position.

In some embodiments, when the amino acid residue at position Z¹² is replaced with an amino acid residue that is different from the amino acid residue recited at said position, the amino acid residue at position Z¹² is replaced with an amino acid residue selected from the group consisting of: A, Achx, Achx(diF), Acpx, Aib, AIB, aMeK, aMeL, Chg, diFCpx, F, L, Pip(NMe), Pip(NMe2), W and diFAchx.

In some embodiments, when the amino acid residue at position Z¹² is replaced with an amino acid residue that is different from the amino acid residue recited at said position, the amino acid residue at position Z¹² is replaced with an amino acid residue comprising at least one quaternary amine selected from the group consisting of: Pip(NMe2).

In some embodiments, when the amino acid residue at position Z¹² is replaced with an amino acid residue that is different from the amino acid residue recited at said position, the amino acid residue at position Z¹² is replaced with an amino acid residue that is THP substituted on the side chain with one or more groups selected from: —OH, —(C₁-C₄) alkyl, —O(C₁-C₄) alkyl, and —CN.

In some embodiments, when the amino acid residue at position Z¹² is replaced with an amino acid residue that is different from the amino acid residue recited at said position, the amino acid residue at position Z¹² is replaced with an aromatic amino acid residue.

In some embodiments, when the amino acid residue at position Z¹² is replaced with an amino acid residue that is different from the amino acid residue recited at said position, the amino acid residue at position Z¹² is replaced with an aliphatic amino acid residue.

In some embodiments, when the amino acid residue at position Z¹² is replaced with an amino acid residue that is different from the amino acid residue recited at said position, the amino acid residue at position Z¹² is replaced with a polar amino acid residue.

In some embodiments, when the amino acid residue at position Z¹² is replaced with an amino acid residue that is different from the amino acid residue recited at said position, the amino acid residue at position Z¹² is replaced with a hydrophilic amino acid residue.

In some embodiments, when the amino acid residue at position Z¹² is replaced with an amino acid residue that is different from the amino acid residue recited at said position, the amino acid residue at position Z¹² is replaced with a basic amino acid residue.

In some embodiments, the amino acid residue at position Z¹² is replaced with an amino acid residue that is different from the amino acid residue recited at said position.

In some embodiments, when the amino acid residue at position Z¹³ is replaced with an amino acid residue that is different from the amino acid residue recited at said position, the amino acid residue at position Z¹³ is replaced with an amino acid residue selected from the group consisting of: K(NMeAC), E, A, AIB, aMeE, APEG2Ser, APEG2Ser(S*), Cit, Dab(NMeAc), Dab(NMecarn), Dab(NMeCOmPEG6), Dab(NMecPEG2a), Dab(NMecPEG3a), Dap(Ac), Dap(NMeAc), E(c), E(C), F, K(5cpa), K(Ac), K(cPEG3a), K(d), K(D), K(dPEG12Ac), K(dPEG6Ac), K(dPEG9Ac), K(Me)₃, K(NMeCOmPEG6), K(NMeCOPEG4N+Me3), K(NMePEG3a), K(NmPEG6Ac), K(PEG2PEG2gEC12), K(PEG2PEG2gEC14), K(PEG2PEG2PEG2gEC12), L, Q(N(Me)2), Tetrazole, Tetrazole(NMe), W, K(DFN), K(IPB) and Nle.

In some embodiments, when the amino acid residue at position Z¹³ is replaced with an amino acid residue that is different from the amino acid residue recited at said position, the amino acid residue at position Z¹³ is replaced with an amino acid residue comprising at least one quaternary amine selected from the group consisting of: APEG2Ser, APEG2Ser(S*), Dab(NMecarn), Dab(NMecPEG2a), Dab(NMecPEG3a), K(5cpa), K(cPEG3a), E(c), E(C), K(d), K(D), K(Me)₃, K(NMeCOPEG4N+Me3) and K(NMePEG3a).

In some embodiments, when the amino acid residue at position Z¹³ is replaced with an amino acid residue that is different from the amino acid residue recited at said position, the amino acid residue at position Z¹³ is replaced with an amino acid residue that is K(NMeAc) substituted on the side chain with one or more groups selected from: —OH, —(C₁-C₄) alkyl, —O(C₁-C₄) alkyl, and —CN.

In some embodiments, when the amino acid residue at position Z¹³ is replaced with an amino acid residue that is different from the amino acid residue recited at said position, the amino acid residue at position Z¹³ is replaced with an aromatic amino acid residue.

In some embodiments, when the amino acid residue at position Z¹³ is replaced with an amino acid residue that is different from the amino acid residue recited at said position, the amino acid residue at position Z¹³ is replaced with an aliphatic amino acid residue.

In some embodiments, when the amino acid residue at position Z¹³ is replaced with an amino acid residue that is different from the amino acid residue recited at said position, the amino acid residue at position Z¹³ is replaced with a hydrophilic amino acid residue.

In some embodiments, when the amino acid residue at position Z¹³ is replaced with an amino acid residue that is different from the amino acid residue recited at said position, the amino acid residue at position Z¹³ is replaced with an acidic amino acid residue.

In some embodiments, the amino acid residue at position Z¹³ is replaced with an amino acid residue that is different from the amino acid residue recited at said position.

In some embodiments, when the amino acid residue at position Z¹⁴ is replaced with an amino acid residue that is different from the amino acid residue recited at said position, the amino acid residue at position Z¹⁴ is replaced with an amino acid residue selected from the group consisting of: K(Ac) and N(NMe).

In some embodiments, when the amino acid residue at position Z¹⁴ is replaced with an amino acid residue that is different from the amino acid residue recited at said position, the amino acid residue at position Z¹⁴ is replaced with an amino acid residue that is N substituted on the side chain with one or more groups selected from: —OH, —(C₁-C₄) alkyl, —O(C₁-C₄) alkyl, and —CN.

In some embodiments, when the amino acid residue at position Z¹⁴ is replaced with an amino acid residue that is different from the amino acid residue recited at said position, the amino acid residue at position Z¹⁴ is replaced with a hydrophilic amino acid residue.

In some embodiments, the amino acid residue at position Z¹⁴ is replaced with an amino acid residue that is different from the amino acid residue recited at said position.

In some embodiments, when the amino acid residue at position Z¹¹ is replaced with an amino acid residue that is different from the amino acid residue recited at said position, the amino acid residue at position Z¹¹ is replaced with an amino acid residue selected from the group consisting of 3pya, 5CF33Pya, 5MePyridinAla, bAla, dK, dL, F, f, H, h, k, N, NMe3Pya, NMebAla, NMeDTyr, orn, Paf, s, t, THP, v, y and A.

In some embodiments, when the amino acid residue at position Z⁵ is replaced with an amino acid residue that is different from the amino acid residue recited at said position, the amino acid residue at position Z⁵ is replaced with an amino acid residue that is 3Pya substituted on the side chain with one or more groups selected from: —OH, —(C₁-C₄) alkyl, —O(C₁-C₄) alkyl, and —CN.

In some embodiments, when the amino acid residue at position Z⁵ is replaced with an amino acid residue that is different from the amino acid residue recited at said position, the amino acid residue at position Z⁵ is replaced with an amino acid residue comprising at least one quaternary amine selected from the group consisting of: NMe3Pya.

In some embodiments, when the amino acid residue at position Z⁵ is replaced with an amino acid residue that is different from the amino acid residue recited at said position, the amino acid residue at position Z⁵ is replaced with an aromatic amino acid residue.

In some embodiments, when the amino acid residue at position Z⁵ is replaced with an amino acid residue that is different from the amino acid residue recited at said position, the amino acid residue at position Z⁵ is replaced with an aliphatic amino acid residue.

In some embodiments, when the amino acid residue at position Z⁵ is replaced with an amino acid residue that is different from the amino acid residue recited at said position, the amino acid residue at position Z⁵ is replaced with a polar amino acid residue.

In some embodiments, when the amino acid residue at position Z⁵ is replaced with an amino acid residue that is different from the amino acid residue recited at said position, the amino acid residue at position Z⁵ is replaced with a hydrophilic amino acid residue.

In some embodiments, when the amino acid residue at position Z¹¹ is replaced with an amino acid residue that is different from the amino acid residue recited at said position, the amino acid residue at position Z¹¹ is replaced with a basic amino acid residue.

In some embodiments, when the amino acid residue at position Z¹⁵ is replaced with an amino acid residue that is different from the amino acid residue recited at said position, the amino acid residue at position Z¹⁵ is replaced with a non-polar amino acid residue.

In some embodiments, the amino acid residue at position Z¹⁵ is replaced with an amino acid residue that is different from the amino acid residue recited at said position.

In some embodiments, when the amino acid residue at position Z¹⁶ is replaced with an amino acid residue that is different from the amino acid residue recited at said position, the amino acid residue at position Z¹⁶ is replaced with an amino acid residue selected from the group consisting of: 4diFPro, NMeDTyr, NMeK(PEG2PEG2C12), NMeK(PEG2PEG2C14), NMeK(PEG2PEG2gEC12), NMeK(PEG2PEG2gEC14), NMeK(PEG2PEG2K(PEG2PEG2gEC12)₂), NMeK(PEG2PEG2K(PEG2PEG2PEG2PEG2gEC12)₂), NMeK(PEG2PEG2PEG2gEC12), NMeK(PEG2PEG2PEG2PEG2gEC12), NMeK(PEG2PEG6gEC12), NMeK(PEG2PEG6gEC14), NMeK(SP6PEG2PEG2C12) and NMeK(SP6PEG2PEG2gEC12).

In some embodiments, when the amino acid residue at position Z¹⁶ is replaced with an amino acid residue that is different from the amino acid residue recited at said position, the amino acid residue at position Z¹⁶ is replaced with an amino acid residue comprising at least one quaternary amine selected from the group consisting of: NMeK(SP6PEG2PEG2C12) and NMeK(SP6PEG2PEG2gEC12).

In some embodiments, when the amino acid residue at position Z¹⁶ is replaced with an amino acid residue that is different from the amino acid residue recited at said position, the amino acid residue at position Z¹⁶ is replaced with an amino acid residue that is Sar substituted on the side chain with one or more groups selected from: —OH, —(C₁-C₄) alkyl, —O(C₁-C₄) alkyl, and —CN.

In some embodiments, when the amino acid residue at position Z¹⁶ is replaced with an amino acid residue that is different from the amino acid residue recited at said position, the amino acid residue at position Z¹⁶ is replaced with an aromatic amino acid residue.

In some embodiments, when the amino acid residue at position Z¹⁶ is replaced with an amino acid residue that is different from the amino acid residue recited at said position, the amino acid residue at position Z¹⁶ is replaced with an acidic amino acid residue.

In some embodiments, when the amino acid residue at position Z¹⁶ is replaced with an amino acid residue that is different from the amino acid residue recited at said position, the amino acid residue at position Z¹⁶ is replaced with a polar amino acid residue.

In some embodiments, when the amino acid residue at position Z¹⁶ is replaced with an amino acid residue that is different from the amino acid residue recited at said position, the amino acid residue at position Z¹⁶ is replaced with a hydrophilic amino acid residue.

In some embodiments, when the amino acid residue at position Z¹⁶ is replaced with an amino acid residue that is different from the amino acid residue recited at said position, the amino acid residue at position Z¹⁶ is replaced with a non-polar amino acid residue.

In some embodiments, the amino acid residue at position Z¹⁶ is replaced with an amino acid residue that is different from the amino acid residue recited at said position.

In some embodiments, the amino acid residue(s) specified at a given position are substituted on the side chain with one or more groups selected from: —OH, —(C₁-C₄) alkyl, —O(C₁-C₄) alkyl, and —CN.

In some embodiments, R^{NT} is selected from the group consisting of: —C(O)-optionally substituted (C₁-C₁₅) alkyl and —C(O)-optionally substituted (C₁-C₃₅) heteroalkyl.

In some embodiments, R^{NT} is selected from the group consisting of: —C(O)-optionally substituted (C₁-C₁₀) alkyl and —C(O)-optionally substituted (C₁-C₃₅) heteroalkyl.

In some embodiments, R^{NT} is selected from the group consisting of: —C(O)-optionally substituted (C₁-C₈) alkyl and —C(O)-optionally substituted (C₁-C₃₀) heteroalkyl.

In some embodiments, R^{NT} is selected from the group consisting of: —C(O)-optionally substituted (C₁—C) alkyl and —C(O)-optionally substituted (C₁-C₃₀) heteroalkyl.

In some embodiments, R^{NT} is selected from the group consisting of: (d) gEPEG2PEG2CO, 4cpgCO, 5cpaCO, AcdPEG12CO, AcdPEG6CO, AcdPEG9CO, CEPEG2PEG2CO, C14gEPEG2PEG2CO, CF3CO, CF3propylamide, cPEG2gCO, cPEG3aCO, cPEG3gCO, cPEG5aCO, EtCO, F3CO, MeCO, mPEG3CO, mPEG6CO, cPEG3AmCO, DFNCO, DFNPEG2PEG2CO, IPBCO and IPBPEG2PEG2CO.

In some embodiments, R^{NT} is an —C(O)-optionally substituted (C₁-C₂₀) alkyl, for example, 4cpgCO, CF3CO, CF3propylamide, EtCO, F3CO, MeCO or 5cpaCO.

In some embodiments, R^{NT} is an —C(O)-optionally substituted (C₁-C₂₀) alkyl that comprises a quaternary amine, for example, 5cpaCO.

In some embodiments, R^{NT} is an —C(O)-optionally substituted (C₁-C₄₀) heteroalkyl, for example, AcdPEG12CO, AcdPEG6CO, AcdPEG9CO, C₁₂gEPEG2PEG2CO, cPEG2gCO, cPEG3gCO, mPEG3CO, mPEG6CO,
(d)gEPEG2PEG2CO, cPEG3aCO, and cPEG5aCO.

In some embodiments, R^{NT} is an —C(O)-optionally substituted (C₁-C₄₀) heteroalkyl that comprises a quaternary amine, for example, (d)gEPEG2PEG2CO, cPEG3aCO, cPEG5aCO.

In some embodiments, R^{NT} is an —C(O)-optionally substituted heteroalkyl that comprises a hydrophilic polymer, for example, polyethylene glycol (PEG).

In some embodiments, R^{NT} is an —C(O)-optionally substituted heteroalkyl that comprises the formula —[O—CH₂CH₂]n-, wherein n is an integer, for example wherein:
(i) n is an integer from about 1 to about 20;
(ii) n is an integer from about 1 to about 15;
(iii) n is an integer from about 1 to about 10; or
(iv) n is an integer from about 2 to about 12.

In some embodiments, when R^{NT} is a —C(O)-optionally substituted heteroalkyl, the —C(O)-optionally substituted heteroalkyl is unsubstituted or is substituted with one or more substituents independently selected from the group consisting of: —OH, —NH₂, —CO₂H, —CO₂CH₃, —NH(C═NH)NH₂, ═O (i.e., two geminal hydrogens on a carbon atom of the heteroalkyl are replaced with the group ═O),

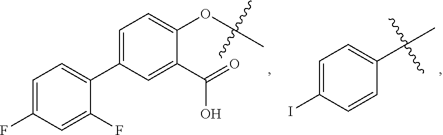 [SMILES: *Oc1ccc(-c2ccc(F)cc2F)cc1C(=O)O ; *c1ccc(I)cc1]

or a quaternary amine as described herein, for example, —N⁺(CH₃)₃.

In some embodiments, when R^{NT} is a —C(O)-optionally substituted alkyl, the —C(O)-optionally substituted alkyl is unsubstituted or is substituted with one or more substituents independently selected from the group consisting of: Fluoro, —NH(C═NH)NH₂,

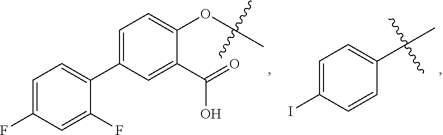 [SMILES: *Oc1ccc(-c2ccc(F)cc2F)cc1C(=O)O ; *c1ccc(I)cc1]

or a quaternary amine as described herein, for example, —N⁺(CH₃)₃.

In some embodiments:
(i) each R^{Y} and R^{Z} is independently selected from the group consisting of: hydrogen, optionally substituted (C₁-C₁₀) alkyl and optionally substituted (C₁-C₂₅) heteroalkyl, or
(ii) R^{Y} and R^{Z} come together with the N atom to which they are attached to form an optionally substituted (C₅₋₇) heterocyclic ring or an optionally substituted (C₇-C₉) bicyclic heterocyclic ring.

In some embodiments:
(i) each R^{Y} and R^{Z} is independently selected from the group consisting of: hydrogen, optionally substituted (C₁-C₈) alkyl and optionally substituted (C₁-C₂₀) heteroalkyl, or (ii) R^{Y} and R^{Z} come together with the N atom to which they are attached to form an optionally substituted (C) heterocyclic ring or an optionally substituted (C₈) bicyclic heterocyclic ring.

In some embodiments, R^{CT} is selected from the group consisting of: CONH2, CO(DiFPip), CO(Morph), CO(mPEG8), CO(NHPEG3a), CO(OAZBO), CO(TFMOHPip), CON(Me)2, CON(MePEG2), CON(mPEG2), CON(NMePip), CONH(PEG3a), CONH(PEG5a), CONHMe and CONMe2.

In some embodiments, R^{CT} is selected from the group consisting of: optionally substituted aminyl, optionally substituted piperidinyl, optionally substituted piperazinyl, optionally substituted morpholinyl and optionally substituted 3-oxa-8-azabicyclo[3.2.1]octanyl.

In some embodiments, R^{CT} is N(R^{Y})(R^{Z}), wherein R^{Y} is hydrogen and R^{Z} is an optionally substituted (C₁-C₃₀) heteroalkyl that comprises a quaternary amine, for example, CO(NHPEG3a), CONH(PEG3a) or CONH(PEG5a).

In some embodiments, when R^{Y} and/or R^{Z} is an optionally substituted alkyl, each optionally substituted alkyl is independently unsubstituted or is substituted with one or more substituents independently selected from the group consisting of: a quaternary amine as described herein, for example, —N⁺(CH₃)₃.

In some embodiments, when R^{Y} and/or R^{Z} is an optionally substituted heteroalkyl, each optionally substituted heteroalkyl is independently unsubstituted or is substituted with one or more substituents independently selected from the group consisting of: a quaternary amine as described herein, for example, —N⁺(CH₃)₃.

In some embodiments, when R^{Y} and R^{Z} come together with the N atom to which they are attached to form an optionally substituted heterocyclic ring, for example, optionally substituted aminyl, optionally substituted piperidinyl, optionally substituted piperazinyl and optionally substituted morpholinyl, the optionally substituted heterocyclic ring is unsubstituted or is substituted with one or more substituents independently selected from the group consisting of: Fluoro, —OH, —CF₃, (C₁-C₃) alkyl, or a quaternary amine as described herein, for example, —N⁺(CH₃)₃.

In some embodiments, when R^{Y} and R^{Z} come together with the N atom to which they are attached to form an optionally substituted bicyclic heterocyclic ring, for example, optionally substituted 3-oxa-8-azabicyclo[3.2.1]octanyl, the optionally substituted bicyclic heterocyclic ring is unsubstituted or is substituted with one or more substituents independently selected from the group consisting of: Fluoro, —OH, —CF₃, (C₁-C₃) alkyl, or a quaternary amine as described herein, for example, —N⁺(CH₃)₃.

In some embodiments, the cyclic peptide further comprises one or more natural or unnatural polymer(s) or a combination(s) thereof.

In some embodiments, the cyclic peptide further comprises a natural or unnatural polymer or a combination thereof.

In some embodiments, the cyclic peptide further comprises one or more natural or unnatural polymer or a combination thereof that is conjugated to R^{NT}, when present, and/or R^{CT}, when present, and/or the amino acid residues at positions Z³, Z⁵, Z⁶, Z⁷, Z⁸, Z¹⁰, Z¹¹, Z¹², Z¹³, Z¹⁴, Z¹⁵ and/or Z¹⁶, when an amino acid residue is present at the position.

In some embodiments, the cyclic peptide further comprises a natural or unnatural polymer or a combination thereof that is conjugated to R^{NT}, when present, or R^{CT}, when present, or the amino acid residues at positions Z³, Z⁵, Z⁶, Z⁷, Z⁸, Z¹⁰, Z¹¹, Z¹², Z¹³, Z¹⁴, Z¹⁵ or Z¹⁶, when an amino acid residue is present at the position.

The properties of the natural or unnatural polymer or a combination thereof can be optimized by modifying the length, conformation (e.g., branched or linear), and/or functionalization (e.g., adding a negatively charged group) of the natural or unnatural polymer or a combination thereof. For example, the natural or unnatural polymer or a combination thereof can be modified to increase solubility, avoid aggregation, and the like of the cyclic peptides of the present invention. For example, the natural or unnatural polymer or a combination thereof can be modified to increase the solubility and avoid aggregation of the cyclic peptides of the present invention by including hydrophilic or water-soluble polymers, including but not limited to PEG, charged PEG molecules, amino acids, or combinations thereof.

In an embodiment, the natural or unnatural polymer or a combination thereof is water-soluble, e.g., so that the cyclic peptides of the present invention do not precipitate out in an aqueous (e.g., physiological) environment. Further, the natural or unnatural polymer or a combination thereof is biocompatible, e.g., does not cause injury, toxicity or an immunological reaction in vivo.

A non-limiting example of a natural polymeric group is an amino acid sequence containing from about 10 to about 30 amino acids derived from (poly)peptides such as, natriuretic peptide precursor C, atrial natriuretic peptide, brain natriuretic peptide, serum albumin, IgG, histidine-rich glycoproteins, fibronectin, fibrinogen, zinc finger-containing polypeptides, osteocrin or fibroblast growth factor 2 (FGF2), or variants thereof with substitutions and/or deletions. A non-limiting example of an unnatural polymeric group (e.g. a synthetic polymer) is polyethylene glycol (PEG, also called polyethylene oxide (PEO)).

In an embodiment, the natural polymer or unnatural polymer comprises residues selected from amino acids or carbohydrates or combinations, thereof. In an embodiment, the natural or unnatural polymer or a combination thereof comprises an amino acid sequence. In an embodiment, the natural or unnatural polymer or a combination thereof comprises about 10 to about 30 amino acid residues. In an embodiment, the natural or unnatural polymer or a combination thereof comprises about 10 to about 25 amino acid residues. In an embodiment, the natural or unnatural polymer or a combination thereof comprises about 15 to about 20 amino acid residues.

In an embodiment, the natural or unnatural polymer or a combination thereof is a combination of a natural polymer and an unnatural polymer, for example a combination of an amino acid sequence and a hydrophilic polymer, such as polyethylene glycol (PEG).

In an embodiment, the natural or unnatural polymer or a combination thereof comprises optionally substituted heteroalkylene. In an embodiment, the natural or unnatural polymer or a combination thereof is optionally substituted heteroalkylene.

In an embodiment, the natural or unnatural polymer or a combination thereof comprises optionally substituted (C₁₀-C₂₀₀) heteroalkylene. In an embodiment, the natural or unnatural polymer or a combination thereof is optionally substituted (C₁₀-C₂₀₀) heteroalkylene. In an embodiment, the natural or unnatural polymer or a combination thereof comprises optionally substituted (C₁₀-C₁₅₀) heteroalkylene. In an embodiment, the natural or unnatural polymer or a combination thereof is optionally substituted (C₁₀-C₁₅₀) heteroalkylene. In an embodiment, the natural or unnatural polymer or a combination thereof comprises optionally substituted (C₁₀-C₁₀₀) heteroalkylene. In an embodiment, the natural or unnatural polymer or a combination thereof is optionally substituted (C₁₀-C₁₀₀) heteroalkylene. In an embodiment, the natural or unnatural polymer or a combination thereof comprises optionally substituted (C₁₀-C₉₀) heteroalkylene. In an embodiment, the natural or unnatural polymer or a combination thereof is optionally substituted (C₁₀-C₉₀) heteroalkylene.

In an embodiment, the natural or unnatural polymer or a combination thereof comprises optionally substituted (C₁₀-C₈₀) heteroalkylene. In an embodiment, the natural or unnatural polymer or a combination thereof is optionally substituted (C₁₀-C₈₀) heteroalkylene. In an embodiment, the natural or unnatural polymer or a combination thereof comprises optionally substituted (C₁₀-C₇₀) heteroalkylene. In an embodiment, the natural or unnatural polymer or a combination thereof is optionally substituted (C₁₀-C₇₀) heteroalkylene. In an embodiment, the natural or unnatural polymer or a combination thereof comprises optionally substituted (C₁₀-C₆₀) heteroalkylene. In an embodiment, the natural or unnatural polymer or a combination thereof is optionally substituted (C₁₀-C₆0) heteroalkylene. In an embodiment, the natural or unnatural polymer or a combination thereof comprises about 1 to about 7 heteroatoms, independently selected from the group consisting of N, O, S, and P, for every 10 carbons in the optionally substituted heteroalkylene. In an embodiment, the natural or unnatural polymer or a combination thereof comprises about 1 to about 5 heteroatoms, independently selected from the group consisting of N, O, S, and P, for every 10 carbons in the optionally substituted heteroalkylene. In an embodiment, the heteroatoms are independently selected from the group consisting of N, O or S. In an embodiment, the heteroatoms are independently selected from the group consisting of N or O.

In an embodiment, the natural or unnatural polymer or a combination thereof is a combination of naturally occurring monomer units and synthetic monomer units, for example a combination of amino acid monomer units and PEG monomer units.

In an embodiment, the natural or unnatural polymer or a combination thereof comprises a hydrophilic polymer. In an embodiment, the natural or unnatural polymer or a combination thereof is a hydrophilic polymer. In an embodiment, the hydrophilic polymer can be branched or unbranched. In an embodiment, the hydrophilic polymer is not branched. The present invention contemplates the use of hydrophilic or water soluble polymers (e.g., PEG) that can vary in type (e.g., homopolymer or copolymer; random, alternating or block copolymer; linear or branched), linkage (e.g., hydrolysable or stable linkage such as, e.g., amide, imine, aminal, alkylene, or ester bond), and length (e.g., from about 0.2, 0.4 or 0.6 kDa to about 2, 3, 4 or 5 kDa, such as about 0.2 kDa to about 5 kDa). Non-limiting examples of hydrophilic polymers that can be used in the present invention include polymers formed from carboxylic acid-bearing monomers (e.g., methacrylic acid (MA) and acrylic acid (AA)), polyvinyl alcohols, polymers formed from hydroxyl-bearing monomers (e.g., hydroxyethyl methacrylate (HEMA), hydroxypropyl methacrylate (HPMA), hydroxypropyl methacrylamide, and 3-trimethylsilylpropyl methacrylate (TMSPMA)), polyalkylene oxides, polyoxyethylated polyols (e.g., glycerol), poly(ethylene glycol) (PEG), poly(propylene glycol), mono-C₁-C₁₀ alkoxy-PEGs (e.g., monomethoxy-PEG), tresyl monomethoxy-PEG, aryloxy-PEGs, PEG acrylate (PEGA), PEG methacrylate, PEG propionaldehyde, bis-succinimidyl carbonate PEG, copolymers of 2-methacryloyloxyethyl-phosphorylcholine (MPC) and N-vinyl pyrrolidone (VP), hydroxy functional poly(N-vinyl pyrrolidone) (PVP), SIS-PEG (SIS is polystyrene-polyisobutylene-polystyrene block copolymer), polystyrene-PEG, polyisobutylene-PEG, PCL-PEG (PCL is polycaprolactone), PLA-PEG (PLA is polylactic acid), PMMA-PEG (PMMA is poly(methyl methacrylate)), PDMS-PEG (PDMS is polydimethyloxanone), PVDF-PEG (PVDF is polyvinylidene fluoride), PLURONIC™ surfactants (polypropylene oxide-co-polyethylene glycol), poly(tetramethylene glycol), poly(L-lysine-g-ethylene glycol) (PLL-g-PEG), poly(L-lysine-g-hyaluronic acid) (PLL-gHA), poly(L-lysine-g-phosphoryl choline) (PLL-g-PC), poly(L-lysine-g-vinyl pyrrolidone) (PLL-g-PVP), poly(ethylimine-g-ethylene glycol) (PEI-g-PEG), poly(ethylimine-g-hyaluronic acid) (PEI-g-HA), poly(ethylimine-g-phosphoryl choline) (PEI-g-PC), poly(ethylimine-g-vinyl pyrrolidone) (PEI-g-PVP), PLL-co-HA, PLL-co-PC, PLL-co-PVP, PEI-co-PEG, PEI-co-HA, PEI-co-PC, PEI-co-PVP, cellulose and derivatives thereof (e.g., hydroxyethyl cellulose), dextran, dextrins, hyaluronic acid and derivatives thereof (e.g., sodium hyaluronate), elastin, chitosan, acrylic sulfate, acrylic sulfonate, acrylic sulfamate, methacrylic sulfate, methacrylic sulfonate, methacrylic sulfamate, polymers and copolymers thereof, and polymers and copolymers of combinations thereof.

In an embodiment, the natural or unnatural polymer or a combination thereof comprises one or more aromatic compound(s), such as dihydroxybenzoic acid (e.g. 3,5-dihydroxybenzoic acid), biphenol (e.g. 4,4′-biphenol) or 4-hydroxybenzyl alcohol, wherein each of the one or more aromatic compound(s) connect(s) two hydrophilic polymers, such as PEG.

In an embodiment, the hydrophilic polymer comprises polyethylene glycol (PEG). In an embodiment, the hydrophilic polymer is PEG. In an embodiment, the hydrophilic polymer comprises PEG18. In an embodiment, the hydrophilic polymer is PEG18. In an embodiment, the hydrophilic polymer comprises PEG21. In an embodiment, the hydrophilic polymer is PEG21. In general, a “PEGn” polymer associated with the number n, comprises the formula: —[O—CH₂CH₂]n-, where n is the number of ethylene oxide units. In an embodiment, the natural or unnatural polymer or a combination thereof comprises the formula: —[O—CH₂CH₂]n-, wherein n is an integer. In an embodiment, n is an integer from about 6 to about 100. In an embodiment, n is an integer from about 6 to about 100, and the PEG polymer is from about 0.3 kDa to about 5 kDa. In another embodiment, n is an integer from about 12 to about 50. In another embodiment, n is an integer from about 12 to about 50, and the PEG polymer is from about 0.6 kDa to about 2.5 kDa. In yet another embodiment, n is an integer from about 12 to about 24. In yet another embodiment, n is an integer from about 12 to about 24, and the PEG polymer is from about 0.6 kDa to about 1.2 kDa.

In an embodiment, the natural or unnatural polymer or a combination thereof is at least about 0.4, 0.6, 0.8, 1, 1.2, 1.4, 1.6 or 1.8 kDa, or up to about 2, 2.2, 2.4, 2.6, 2.8, 3, 3.2, 3.4, 3.6, 3.8, 4, 4.2, 4.4, 4.6, 4.8 or 5 kDa. In an additional embodiment, the natural or unnatural polymer or a combination thereof is in the range from about 0.4 kDa to about 2.5 kDa or from about 0.6 kDa to about 1.5 kDa. In an embodiment, the natural or unnatural polymer or a combination thereof is at least about 0.2 kDa. In an embodiment, the natural or unnatural polymer or a combination thereof is at least about 0.4 kDa. In an embodiment, the natural or unnatural polymer or a combination thereof is at least about 0.6 kDa. In an embodiment, the natural or unnatural polymer or a combination thereof is less than about 2 kDa. In an embodiment, the natural or unnatural polymer or a combination thereof is less than about 3 kDa. In an embodiment, the natural or unnatural polymer or a combination thereof is less than about 4 kDa. In an embodiment, the natural or unnatural polymer or a combination thereof is less than about 5 kDa. In an embodiment, the natural or unnatural polymer or a combination thereof is about 0.2 kDa to about 5 kDa. In an embodiment, the natural or unnatural polymer or a combination thereof is about 0.4 kDa to about 5 kDa. In an embodiment, the natural or unnatural polymer or a combination thereof is about 0.6 kDa to about 5 kDa. In an embodiment, the natural or unnatural polymer or a combination thereof is about 0.2 kDa to about 2 kDa. In an embodiment, the natural or unnatural polymer or a combination thereof is about 0.2 kDa to about 3 kDa. In an embodiment, the natural or unnatural polymer or a combination thereof is about 0.2 kDa to about 4 kDa. In an embodiment, the natural or unnatural polymer or a combination thereof is about 0.4 kDa to about 2 kDa. In an embodiment, the natural or unnatural polymer or a combination thereof is about 0.4 kDa to about 3 kDa. In an embodiment, the natural or unnatural polymer or a combination thereof is about 0.4 kDa to about 4 kDa. In an embodiment, the natural or unnatural polymer or a combination thereof is about 0.6 kDa to about 2 kDa. In an embodiment, the natural or unnatural polymer or a combination thereof is about 0.6 kDa to about 3 kDa. In an embodiment, the natural or unnatural polymer or a combination thereof is about 0.6 kDa to about 4 kDa. In an embodiment, the size of the natural or unnatural polymer or a combination thereof in kDa can be determined using mass spectrometry. In an embodiment, the size of the natural or unnatural polymer or a combination thereof in kDa is determined using mass spectrometry.

In an embodiment, the natural or unnatural polymer or a combination thereof (for example, a hydrophilic or water-soluble polymer (e.g., PEG)) is functionalized with one or more functional groups that impart a negative charge to the natural or unnatural polymer or a combination thereof under physiological conditions, such as, e.g, carboxyl, sulfate or phosphate groups, or a combination thereof. In an embodiment, the natural or unnatural polymer or a combination thereof (for example, a hydrophilic or water-soluble polymer (e.g., PEG)) is functionalized with one or more functional groups that impart a positive charge to the polymer under physiological conditions, such as, an amine, or a tertiary amine.

In an embodiment, the natural or unnatural polymer or a combination thereof comprises one or more cleavable groups, such as disulfide, ester and/or carbonate.

In some embodiments, the cyclic peptide is an inhibitor of an interleukin-23 (IL-23) receptor.

In some embodiments, the cyclic peptide inhibits the binding of an interleukin-23 (IL-23) to an IL-23 receptor.

In some embodiments, the cyclic peptide inhibits an interleukin-23 (IL-23) receptor to reduce or prevent the activation of the Jak-Stat signaling molecules, Jak2, Tyk2, Stat1, Stat 3, Stat 4, and/or Stat 5.

In some embodiments, the cyclic peptide inhibits an interleukin-23 (IL-23) receptor to reduce or prevent the phosphorylation of Stat 3.

In some embodiments, the cyclic peptide inhibits an interleukin-23 (IL-23) receptor in a reporter assay with an IC₅₀ value less than about 6.3 μM. In some embodiments, the cyclic peptide inhibits an interleukin-23 (IL-23) receptor in a reporter assay with an IC₅₀ value less than about 2 μM. In some embodiments, the cyclic peptide inhibits an interleukin-23 (IL-23) receptor in a reporter assay with an IC₅₀ value less than about 1 μM. In some embodiments, the cyclic peptide inhibits an interleukin-23 (IL-23) receptor in a reporter assay with an IC₅₀ value less than about 0.5 μM. In some embodiments, the cyclic peptide inhibits an interleukin-23 (IL-23) receptor in a reporter assay with an IC₅₀ value less than about 0.25 μM. In some embodiments, the cyclic peptide inhibits an interleukin-23 (IL-23) receptor in a reporter assay with an IC₅₀ value less than about 0.1 μM. In some embodiments, the cyclic peptide inhibits an interleukin-23 (IL-23) receptor in a reporter assay with an IC₅₀value less than about 0.05 μM. In some embodiments, the cyclic peptide inhibits an interleukin-23 (IL-23) receptor in a reporter assay with an IC₅₀ value less than about 0.025 μM. In some embodiments, the cyclic peptide inhibits an interleukin-23 (IL-23) receptor in a reporter assay with an IC₅₀ value less than about 0.01 μM. In some embodiments, the cyclic peptide inhibits an interleukin-23 (IL-23) receptor in an IL-23 reporter assay with an IC₅₀ value less than about 0.005 μM. In some embodiments, the cyclic peptide inhibits an interleukin-23 (IL-23) receptor in a reporter assay with an IC₅₀ value less than about 0.0025 μM.

In some embodiments, the IL-23 reporter assay is as described in the description In some embodiments, the cyclic peptide inhibits an interleukin-23 (IL-23) receptor in a peripheral blood mononuclear cell (PBMC) pSTAT3 assay with an IC₅₀ value less than about 0.05 nM. In some embodiments, the cyclic peptide inhibits an interleukin-23 (IL-23) receptor in a peripheral blood mononuclear cell (PBMC) pSTAT3 assay with an IC₅₀ value less than about 0.04 nM. In some embodiments, the cyclic peptide inhibits an interleukin-23 (IL-23) receptor in a peripheral blood mononuclear cell (PBMC) pSTAT3 assay with an IC₅₀ value less than about 0.03 nM.

In some embodiments, the cyclic peptide inhibits an interleukin-23 (IL-23) receptor in a peripheral blood mononuclear cell (PBMC) pSTAT3 assay with an IC₅₀ value less than about 0.02 nM In some embodiments, the cyclic peptide inhibits an interleukin-23 (IL-23) receptor in a peripheral blood mononuclear cell (PBMC) pSTAT3 assay with an IC₅₀ value less than about 0.01 nM. In some embodiments, the peripheral blood mononuclear cell (PBMC) pSTAT3 assay is as described in the description.

In some embodiments, the cyclic peptide exhibits an Exposed Polar Surface Area (EPSA) of less than about 275 Å² in an EPSA assay. In some embodiments, the cyclic peptide exhibits an Exposed Polar Surface Area (EPSA) of less than about 250 Å² in an EPSA assay. In some embodiments, the cyclic peptide exhibits an Exposed Polar Surface Area (EPSA) of less than about 225 Å² in an EPSA assay. In some embodiments, the cyclic peptide exhibits an Exposed Polar Surface Area (EPSA) of less than about 200 Å² in an EPSA assay. In some embodiments, the cyclic peptide exhibits an Exposed Polar Surface JaneArea (EPSA) of less than about 175 Å² in an EPSA assay. In some embodiments, the cyclic peptide exhibits an Exposed Polar Surface Area (EPSA) of less than about 150 Å² in an EPSA assay. In some embodiments, the cyclic peptide exhibits an Exposed Polar Surface Area (EPSA) of less than about 145 Å² in an EPSA assay. In some embodiments, the cyclic peptide exhibits an Exposed Polar Surface Area (EPSA) of less than about 140 Å² in an EPSA assay. In some embodiments, the Exposed Polar Surface Area (EPSA) assay is as described in the description.

In another general aspect, the invention relates to a compound or compounds selected from any one of the tables 1A, 1B, 1C, 1D, lE and 1 For pharmaceutically acceptable salts thereof or solvates thereof. In some embodiments, the cyclic peptide has the following structure or pharmaceutically acceptable salts thereof or solvates thereof:

In another general aspect, the invention relates to a pharmaceutical composition comprising the cyclic peptide or pharmaceutically acceptable salt thereof or solvate thereof as described herein, and a pharmaceutically acceptable carrier, excipient, or diluent.

In some embodiments, the pharmaceutical composition further comprises an enteric coating.

In some embodiments, the enteric coating protects and releases the pharmaceutical composition within a subject's lower gastrointestinal system.

In another general aspect, the invention relates to a method for treating an Inflammatory Bowel Disease (IBD), ulcerative colitis, Crohn's disease, Celiac disease (nontropical Sprue), enteropathy associated with seronegative arthropathies, microscopic colitis, collagenous colitis, eosinophilic gastroenteritis, colitis associated with radio- or chemo-therapy, colitis associated with disorders of innate immunity as in leukocyte adhesion deficiency-1, chronic granulomatous disease, glycogen storage disease type ib, Hermansky-Pudlak syndrome, Chediak-Higashi syndrome, and Wiskott-Aldrich Syndrome, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, psoriasis, psoriatic arthritis, or graft versus host disease in a subject, comprising providing to the subject an effective amount of the cyclic peptide or pharmaceutically acceptable salt thereof or solvate thereof as described herein, or the pharmaceutical composition as described herein.

In some embodiments of the method described herein, the cyclic peptide or pharmaceutically acceptable salt thereof or solvate thereof or the pharmaceutical composition is provided to the subject by an oral, parenteral, intravenous, peritoneal, intradermal, subcutaneous, intramuscular, intrathecal, inhalation, vaporization, nebulization, sublingual, buccal, parenteral, rectal, intraocular, inhalation, topically, vaginal, or topical route of administration.

In some embodiments of the method described herein for treating Inflammatory Bowel Disease (IBD), ulcerative colitis, or Crohn's disease, the cyclic peptide or pharmaceutically acceptable salt thereof or solvate thereof or the pharmaceutical composition is provided to the subject orally.

In some embodiments of the method described herein for treating psoriasis, the cyclic peptide or pharmaceutically acceptable salt thereof or solvate thereof or the pharmaceutical composition is provided to the subject orally, topically, parenterally, intravenously, subcutaneously, peritonealy, or intravenously.

In some embodiments, the cyclic peptide described herein, or the pharmaceutical composition described herein for use in the treatment of an Inflammatory Bowel Disease (IBD), ulcerative colitis, Crohn's disease, Celiac disease (nontropical Sprue), enteropathy associated with seronegative arthropathies, microscopic colitis, collagenous colitis, eosinophilic gastroenteritis, colitis associated with radio- or chemo-therapy, colitis associated with disorders of innate immunity as in leukocyte adhesion deficiency-1, chronic granulomatous disease, glycogen storage disease type 1b, Hermansky-Pudlak syndrome, Chediak-Higashi syndrome, and Wiskott-Aldrich Syndrome, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, psoriasis, psoriatic arthritis, or graft versus host disease in a subject, comprising providing to the subject an effective amount of the cyclic peptide or pharmaceutically acceptable salt thereof or solvate thereof as described herein, or the pharmaceutical composition as described herein.

In some embodiments, the cyclic peptide or pharmaceutically acceptable salt thereof or solvate thereof or the pharmaceutical composition is provided to the subject by an oral, parenteral, intravenous, peritoneal, intradermal, subcutaneous, intramuscular, intrathecal, inhalation, vaporization, nebulization, sublingual, buccal, parenteral, rectal, intraocular, inhalation, topically, vaginal, or topical route of administration.

In some embodiments, when the cyclic peptide for use or the pharmaceutical composition for use as described herein is used in the treatment of Inflammatory Bowel Disease (IBD), ulcerative colitis, or Crohn's disease, the cyclic peptide or pharmaceutically acceptable salt thereof or solvate thereof or the pharmaceutical composition is provided to the subject orally.

In some embodiments, when the cyclic peptide or the pharmaceutical composition for use as described herein is used in the treatment of psoriasis, the cyclic peptide or pharmaceutically acceptable salt thereof or solvate thereof or the pharmaceutical composition is provided to the subject orally, topically, parenterally, intravenously, subcutaneously, peritonealy, or intravenously.

In some embodiments, the cyclic peptide, or the pharmaceutical composition as described herein is for use as a medicament.

In some embodiments, the use of the cyclic peptide or the pharmaceutical composition as described herein is for the manufacture of a medicament for treating a disease.

In some embodiments, the use of the cyclic peptide or the pharmaceutical composition as described herein is for the manufacture of a medicament for treating a disease disclosed herein.

### Particular Cyclic Peptides of the Present Invention

In particular, the present invention relates to a cyclic peptide, or a pharmaceutically acceptable salt or solvate thereof, comprising an amino acid sequence of Formula (A).

(A)
Z³-Z⁴-Z⁵-Z⁶-Z⁷-Z⁸-Z⁹-Z¹⁰-Z¹¹-Z¹²-Z¹³-Z¹⁴-Z¹⁵-Z¹⁶,

wherein:
the amino acid residue at position Z³ is absent or the residue of r;
the amino acid residue at position Z⁴ is the residue of an amino acid that is connected to the amino acid residue at position Z⁹;
the amino acid residue at position Z⁵ is the residue of N(N(Me)₂);
the amino acid residue at position Z⁶ is the residue of T;
the amino acid residue at position Z⁷ is the residue of 7MeW;
the amino acid residue at position Z⁸ is the residue of K(NMeAc);
the amino acid residue at position Z⁹ is the residue of an amino acid that is connected to the amino acid residue at position Z⁴;
the amino acid residue at position Z¹⁰ is the residue of TMAPF;
the amino acid residue at position Z¹¹ is the residue of 2Nal;
the amino acid residue at position Z¹² is absent or the residue of THP;
the amino acid residue at position Z¹³ is the residue of K(NMeAc);
the amino acid residue at position Z¹⁴ is the residue of N;
the amino acid residue at position Z¹⁵ is absent or the residue of 3Pya;
the amino acid residue at position Z¹⁶ is absent or the residue of Sar;
eight or fewer of the amino acid residues at positions Z³, Z⁵, Z⁶, Z⁷, Z⁸, Z°, Z¹¹, Z¹², Z¹³, Z¹⁴, Z¹⁵ and Z¹⁶, when an amino acid residue is present at the position, are independently replaced with an amino acid residue that is different from the amino acid residue recited at said position;
wherein when the amino acid residue at position Z³ is replaced with an amino acid residue that is different from the amino acid residue recited at said position, the amino acid residue at position Z³ is replaced with an amino acid residue selected from the group consisting of: APEG2ser, APEG2Ser, APEG2Ser(S*), e(c), e(C), hk(Me)₃, k(5cpa), k(cPEG3a), k(d), k(D), k(dPEG12Ac), k(dPEG6Ac), k(dPEG9Ac), k(Me)₃, K(Me)₃, k(PEG2PEG2gEC12), k(PEG2PEG2gEC14), k(PEG2PEG2PEG2PEG2gEC12), k(PEG2PEG6gEC12), SP6, APEG2Ser(RS), gPEG2Ser and k(PEG2PEG2gE(c)C12;
wherein when the amino acid residue at position Z⁵ is replaced with an amino acid residue that is different from the amino acid residue recited at said position, the amino acid residue at position Z⁵ is replaced with an amino acid residue selected from the group consisting of: A, APEG2Ser(S*), Dab(Me)₃, F, Gab, K(cPEG3a), K(Me)₃, K(PEG2PEG2gEC12), K(PEG2PEG2gEC14), L, N, N(N(Me)), N(NMe), Q and W;
wherein when the amino acid residue at position Z⁶ is replaced with an amino acid residue that is different from the amino acid residue recited at said position, the amino acid residue at position Z⁶ is replaced with an amino acid residue selected from the group consisting of: A and L;
wherein when the amino acid residue at position Z⁷ is replaced with an amino acid residue that is different from the amino acid residue recited at said position, the amino acid residue at position Z⁷ is replaced with an amino acid residue selected from the group consisting of: W, 7(3NAcPh)W, 7CF3W, NMe7MeW, 2Nal, A, F and L;
wherein when the amino acid residue at position Z⁸ is replaced with an amino acid residue that is different from the amino acid residue recited at said position, the amino acid residue at position Z⁸ is replaced with an amino acid residue selected from the group consisting of: K(NMeAC), Q, 4AmPhe, A, AIB, APEG2Ser, APEG2Ser(R*), APEG2Ser(S*), Cit, Dab(NMeAc), Dab(NMecarn), Dab(NMeCarn), Dab(NMeCOmPEG6), Dab(NMecPEG2a), Dab(NMecPEG3a), Dab(NMecPEG5a), Dap(NMeAc), F, K(4cpg), K(Ac), K(cPEG3a), K(Me)₃, K(NMeCOmPEG6), K(NMePEG3a), K(NmPEG6Ac), K(PEG2PEG2gEC12), K(PEG2PEG2gEC14), L, Paf(Ac), Q(N(Me)2), Q(NHtBu), W, Y and K(cPEG3aCO);
wherein when the amino acid residue at position Z¹⁰ is replaced with an amino acid residue that is different from the amino acid residue recited at said position, the amino acid residue at position Z¹⁰ is replaced with an amino acid residue selected from the group consisting of: AEF, 4DMPEF, 4DMPzEF, 4TMABYF, ACHMF, AEF((Ch)cPEG3a), AEF(Ac), AEF(AcCh), AEF(aPEG2a), AEF(BisMEP), AEF(BisMEPa), AEF(BisPEG2a)(RS), AEF(BisPEG2a)(S*), AEF(G), AEF(Me)2, AEF(MEP), AEF(MePrpa), AEF(N(Me)2), AEF(NHCh), AEF(NHcPEG3a), AEF(NMe), AEF(NMe2mPEG3), AEF(NMe3), AEF(NMeBismPEG3), AEF(NMePEG2a), AEF(NmPEG6), AEF(NsCh), AEF(PEG2a), AEF(SPD), APEG2F, APEG3F, dFPPEG3F, Diazabiclyclooctane6F, DMMMF, DMPMF, DMTASF, F(4G), F(4N3), F(4TzlDMA4mPEG), F(4TzlMME), F(4TzlMMol), F(4TzlMMo3), F(4TzlMMo4), F(4TzlTMA1), F(4TzlTMA2), F(4TzlTMA3), F(4TzlTMA4), F(4TzlTMA5), GPEG3F, hFTMAPF, MMoEF, MMoPF, MMPEG3F, morfTMA4F, mPEG2TMA2F, mPEG2TMA4F, mPEG3TMA4F, MPzPEG3F, MTASF, NPyEF, NPyPEG3F, PiperazinequatF, TBAPEG3F, TMA3F, TMA4F, TMA6F, TMA8F, Tzl(Ch), TzlChmPEG, TzlChmPEG3, Y(C₉₀H), Y(OEOXIMECh), Y(OTzlCh), Y(OTzlChC16), Y(OTzlChC8), Y(OTzlC1aC8), Y(OTzlChmPEG), Y(OTzlChmPEG3), Y(OTzlPEG3a), Y(OTzlPEG4a), Y(OTzlTMA4), Y(OZOXIMECh), YC8CO(NHPEG3a), YC8COPip, YCF2H, ACHMF(R*, S*), ACHMF(S*, S*), AEF(cPEG3a), APF, F(4TzlAme2), F(4TzlG2), F(4TzlMMo7) and F(4TzlTMA7);
wherein when the amino acid residue at position Z¹¹ is replaced with an amino acid residue that is different from the amino acid residue recited at said position, the amino acid residue at position Z¹¹ is replaced with an amino acid residue selected from the group consisting of: A, F, L and W;
wherein when the amino acid residue at position Z¹² is replaced with an amino acid residue that is different from the amino acid residue recited at said position, the amino acid residue at position Z¹² is replaced with an amino acid residue selected from the group consisting of: A, Achx, Achx(diF), Acpx, Aib, AIB, aMeK, aMeL, Chg, diFCpx, F, L, Pip(NMe), Pip(NMe2), W and diFAchx;
wherein when the amino acid residue at position Z¹³ is replaced with an amino acid residue that is different from the amino acid residue recited at said position, the amino acid residue at position Z¹³ is replaced with an amino acid residue selected from the group consisting of: K(NMeAC), E, A, AIB, aMeE, APEG2Ser, APEG2Ser(S*), Cit, Dab(NMeAc), Dab(NMecarn), Dab(NMeCOmPEG6), Dab(NMecPEG2a), Dab(NMecPEG3a), Dap(Ac), Dap(NMeAc), E(c), E(C), F, K(5cpa), K(Ac), K(cPEG3a), K(d), K(D), K(dPEG12Ac), K(dPEG6Ac), K(dPEG9Ac), K(Me)₃, K(NMeCOmPEG6), K(NMeCOPEG4N+Me3), K(NMePEG3a), K(NmPEG6Ac), K(PEG2PEG2gEC12), K(PEG2PEG2gEC14), K(PEG2PEG2PEG2gEC12), L, Q(N(Me)2), Tetrazole, Tetrazole(NMe), W, K(DFN), K(IPB) and Nle;
wherein when the amino acid residue at position Z¹⁴ is replaced with an amino acid residue that is different from the amino acid residue recited at said position, the amino acid residue at position Z¹⁴ is replaced with an amino acid residue selected from the group consisting of: K(Ac) and N(NMe);
wherein when the amino acid residue at position Z¹⁵ is replaced with an amino acid residue that is different from the amino acid residue recited at said position, the amino acid residue at position Z¹⁵ is replaced with an amino acid residue selected from the group consisting of: 3pya, 5CF33Pya, 5MePyridinAla, bAla, dK, dL, F, f, H, h, k, N, NMe3Pya, NMebAla, NMeDTyr, orn, Paf, s, t, THP, v, y and A;
wherein when the amino acid residue at position Z¹⁶ is replaced with an amino acid residue that is different from the amino acid residue recited at said position, the amino acid residue at position Z¹⁶ is replaced with an amino acid residue selected from the group consisting of: 4diFPro, NMeDTyr, NMeK(PEG2PEG2C12), NMeK(PEG2PEG2C14), NMeK(PEG2PEG2gEC12), NMeK(PEG2PEG2gEC14), NMeK(PEG2PEG2K(PEG2PEG2gEC12)₂), NMeK(PEG2PEG2K(PEG2PEG2PEG2PEG2gEC12)₂), NMeK(PEG2PEG2PEG2gEC12), NMeK(PEG2PEG2PEG2PEG2gEC12), NMeK(PEG2PEG6gEC12), NMeK(PEG2PEG6gEC14), NMeK(SP6PEG2PEG2C12) and NMeK(SP6PEG2PEG2gEC12); and at least one of the amino acid residues at positions Z³, Z⁵, Z⁶, Z⁷, Z⁸, Z¹⁰, Z¹¹, Z¹², Z¹³, Z¹⁴, Z¹⁵ and/or Z¹⁶, when an amino acid residue is present at the position, each independently comprise(s) at least one quaternary amine.

In particular, the present invention relates to a cyclic peptide, or a pharmaceutically acceptable salt or solvate thereof, comprising an amino acid sequence of Formula (B):

(B)
Z³-Z⁴-Z⁵-Z⁶-Z⁷-Z⁸-Z⁹-Z¹⁰-Z¹¹-Z¹²-Z¹³-Z¹⁴-Z¹⁵-Z¹⁶,

wherein:
the amino acid residue at position Z³ is absent or the residue of r;
the amino acid residue at position Z⁴ is the residue of an amino acid that is connected to the amino acid residue at position Z⁹;
the amino acid residue at position Z⁵ is the residue of N(N(Me)₂);
the amino acid residue at position Z⁶ is the residue of T;
the amino acid residue at position Z⁷ is the residue of 7MeW;
the amino acid residue at position Z⁸ is the residue of K(NMeAc);
the amino acid residue at position Z⁹ is the residue of an amino acid that is connected to the amino acid residue at position Z⁴;
the amino acid residue at position Z¹⁰ is the residue of TMAPF;
the amino acid residue at position Z¹¹ is the residue of 2Nal;
the amino acid residue at position Z¹² is absent or the residue of THP;
the amino acid residue at position Z¹³ is the residue of K(NMeAc);
the amino acid residue at position Z¹⁴ is the residue of N;
the amino acid residue at position Z¹⁵ is absent or the residue of 3Pya;
the amino acid residue at position Z¹⁶ is absent or the residue of Sar;
eight or fewer of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position;
wherein when the amino acid residue at position Z³ is replaced with an amino acid residue that is different from the amino acid residue recited at said position, the amino acid residue at position Z³ is replaced with an amino acid residue selected from the group consisting of: APEG2ser, APEG2Ser, APEG2Ser(S*), e(c), e(C), hk(Me)₃, k(5cpa), k(cPEG3a), k(d), k(D), k(dPEG12Ac), k(dPEG6Ac), k(dPEG9Ac), k(Me)₃, K(Me)₃, k(PEG2PEG2gEC12), k(PEG2PEG2gEC14), k(PEG2PEG2PEG2PEG2gEC12), k(PEG2PEG6gEC12), SP6, APEG2Ser(RS), gPEG2Ser and k(PEG2PEG2gE(c)C12;
wherein when the amino acid residue at position Z⁵ is replaced with an amino acid residue that is different from the amino acid residue recited at said position, the amino acid residue at position Z⁵ is replaced with an amino acid residue selected from the group consisting of: A, APEG2Ser(S*), Dab(Me)₃, F, Gab, K(cPEG3a), K(Me)₃, K(PEG2PEG2gEC12), K(PEG2PEG2gEC14), L, N, N(N(Me)), N(NMe), Q and W;
wherein when the amino acid residue at position Z⁶ is replaced with an amino acid residue that is different from the amino acid residue recited at said position, the amino acid residue at position Z⁶ is replaced with an amino acid residue selected from the group consisting of: A and L;
wherein when the amino acid residue at position Z⁷ is replaced with an amino acid residue that is different from the amino acid residue recited at said position, the amino acid residue at position Z⁷ is replaced with an amino acid residue selected from the group consisting of: W, 7(3NAcPh)W, 7CF3W, NMe7MeW, 2Nal, A, F and L;
wherein when the amino acid residue at position Z⁸ is replaced with an amino acid residue that is different from the amino acid residue recited at said position, the amino acid residue at position Z⁸ is replaced with an amino acid residue selected from the group consisting of: K(NMeAC), Q, 4AmPhe, A, AIB, APEG2Ser, APEG2Ser(R*), APEG2Ser(S*), Cit, Dab(NMeAc), Dab(NMecarn), Dab(NMeCarn), Dab(NMeCOmPEG6), Dab(NMecPEG2a), Dab(NMecPEG3a), Dab(NMecPEG5a), Dap(NMeAc), F, K(4cpg), K(Ac), K(cPEG3a), K(Me)₃, K(NMeCOmPEG6), K(NMePEG3a), K(NmPEG6Ac), K(PEG2PEG2gEC12), K(PEG2PEG2gEC14), L, Paf(Ac), Q(N(Me)2), Q(NHtBu), W, Y and K(cPEG3aCO);
wherein when the amino acid residue at position Z¹⁰ is replaced with an amino acid residue that is different from the amino acid residue recited at said position, the amino acid residue at position Z¹⁰ is replaced with an amino acid residue selected from the group consisting of: AEF, 4DMPEF, 4DMPzEF, 4TMABYF, ACHMF, AEF((Ch)cPEG3a), AEF(Ac), AEF(AcCh), AEF(aPEG2a), AEF(BisMEP), AEF(BisMEPa), AEF(BisPEG2a)(RS), AEF(BisPEG2a)(S*), AEF(G), AEF(Me)2, AEF(MEP), AEF(MePrpa), AEF(N(Me)2), AEF(NHCh), AEF(NHcPEG3a), AEF(NMe), AEF(NMe2mPEG3), AEF(NMe3), AEF(NMeBismPEG3), AEF(NMePEG2a), AEF(NmPEG6), AEF(NsCh), AEF(PEG2a), AEF(SPD), APEG2F, APEG3F, dFPPEG3F, Diazabiclyclooctane6F, DMMMF, DMPMF, DMTASF, F(4G), F(4N3), F(4TzlDMA4mPEG), F(4TzlMME), F(4TzlMMo1), F(4TzlMMo3), F(4TzlMMo4), F(4TzlTMA1), F(4TzlTMA2), F(4TzlTMA3), F(4TzlTMA4), F(4TzlTMA5), GPEG3F, hFTMAPF, MMoEF, MMoPF, MMPEG3F, morfTMA4F, mPEG2TMA2F, mPEG2TMA4F, mPEG3TMA4F, MPzPEG3F, MTASF, NPyEF, NPyPEG3F, PiperazinequatF, TBAPEG3F, TMA3F, TMA4F, TMA6F, TMA8F, Tzl(Ch), TzlChmPEG, TzlChmPEG3, Y(C₉₀H), Y(OEOXIMECh), Y(OTzlCh), Y(OTzlChC16), Y(OTzlChC8), Y(OTzlC1aC8), Y(OTzlChmPEG), Y(OTzlChmPEG3), Y(OTzlPEG3a), Y(OTzlPEG4a), Y(OTzlTMA4), Y(OZOXIMECh), YC8CO(NHPEG3a), YC8COPip, YCF2H, ACHMF(R*, S*), ACHMF(S*, S*), AEF(cPEG3a), APF, F(4TzlAme2), F(4TzlG2), F(4TzlMMo7) and F(4TzlTMA7);
wherein when the amino acid residue at position Z¹¹ is replaced with an amino acid residue that is different from the amino acid residue recited at said position, the amino acid residue at position Z¹¹ is replaced with an amino acid residue selected from the group consisting of: A, F, L and W;
wherein when the amino acid residue at position Z¹² is replaced with an amino acid residue that is different from the amino acid residue recited at said position, the amino acid residue at position Z¹² is replaced with an amino acid residue selected from the group consisting of: A, Achx, Achx(diF), Acpx, Aib, AIB, aMeK, aMeL, Chg, diFCpx, F, L, Pip(NMe), Pip(NMe2), W and diFAchx;
wherein when the amino acid residue at position Z¹³ is replaced with an amino acid residue that is different from the amino acid residue recited at said position, the amino acid residue at position Z¹³ is replaced with an amino acid residue selected from the group consisting of: K(NMeAC), E, A, AIB, aMeE, APEG2Ser, APEG2Ser(S*), Cit, Dab(NMeAc), Dab(NMecarn), Dab(NMeCOmPEG6), Dab(NMecPEG2a), Dab(NMecPEG3a), Dap(Ac), Dap(NMeAc), E(c), E(C), F, K(5cpa), K(Ac), K(cPEG3a), K(d), K(D), K(dPEG12Ac), K(dPEG6Ac), K(dPEG9Ac), K(Me)₃, K(NMeCOmPEG6), K(NMeCOPEG4N+Me3), K(NMePEG3a), K(NmPEG6Ac), K(PEG2PEG2gEC12), K(PEG2PEG2gEC14), K(PEG2PEG2PEG2gEC12), L, Q(N(Me)2), Tetrazole, Tetrazole(NMe), W, K(DFN), K(IPB) and Nle;
wherein when the amino acid residue at position Z¹⁴ is replaced with an amino acid residue that is different from the amino acid residue recited at said position, the amino acid residue at position Z¹⁴ is replaced with an amino acid residue selected from the group consisting of: K(Ac) and N(NMe);
wherein when the amino acid residue at position Z¹⁵ is replaced with an amino acid residue that is different from the amino acid residue recited at said position, the amino acid residue at position Z¹⁵ is replaced with an amino acid residue selected from the group consisting of: 3pya, 5CF33Pya, 5MePyridinAla, bAla, dK, dL, F, f, H, h, k, N, NMe3Pya, NMebAla, NMeDTyr, orn, Paf, s, t, THP, v, y and A;
wherein when the amino acid residue at position Z¹⁶ is replaced with an amino acid residue that is different from the amino acid residue recited at said position, the amino acid residue at position Z¹⁶ is replaced with an amino acid residue selected from the group consisting of: 4diFPro, NMeDTyr, NMeK(PEG2PEG2C12), NMeK(PEG2PEG2C14), NMeK(PEG2PEG2gEC12), NMeK(PEG2PEG2gEC14), NMeK(PEG2PEG2K(PEG2PEG2gEC12)₂), NMeK(PEG2PEG2K(PEG2PEG2PEG2PEG2gEC12)₂), NMeK(PEG2PEG2PEG2gEC12), NMeK(PEG2PEG2PEG2PEG2gEC12), NMeK(PEG2PEG6gEC12), NMeK(PEG2PEG6gEC14), NMeK(SP6PEG2PEG2C12) and NMeK(SP6PEG2PEG2gEC12); and
at least one of the amino acid residues at positions Z³, Z⁵, Z⁶, Z⁷, Z⁸, Z¹⁰, Z¹¹, Z¹², Z¹³, Z¹⁴, Z¹⁵ and/or Z¹⁶, when an amino acid residue is present at the position, each independently comprise(s) at least one masked amine and/or masked amide.

### II. DEFINITIONS

Unless otherwise defined herein, scientific and technical terms used in this application shall have the meanings that are commonly understood by those of ordinary skill in the art.

“About” when referring to a value includes the stated value +/−10% of the stated value. For example, about 50% includes a range of from 45% to 55%, while about 20 molar equivalents includes a range of from 18 to 22 molar equivalents. Accordingly, when referring to a range, “about” refers to each of the stated values +/−10% of the stated value of each end of the range. For instance, a ratio of from about 1 to about 3 (weight/weight) includes a range of from 0.9 to 3.3.

“Patient” or “subject”, which are used interchangably, refer to a living organism, which includes, but is not limited to a human subject suffering from or prone to a disease or condition that can be treated by administration of a pharmaceutical composition as provided herein. Further non-limiting examples may include, but is not limited to humans, other mammals, bovines, rats, mice, dogs, monkeys, goat, sheep, cows, deer, horse, and other mammalian animals and the like. In some aspects, the patient is human.

Unless indicated otherwise the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975). To the extent that the names and abbreviations of amino acids and aminoacyl residues employed in this specification and appended claims differ from those suggestions, they will be made clear to the reader. In sequences of amino acids that represent IL-23 inhibitors the individual amino acids are separated by a hyphen “−” or brackets e.g, lysine is shown as [K].

Throughout the present specification, unless naturally occurring amino acids are referred to by their full name (e.g., alanine, arginine, etc.), they are designated by their conventional three-letter or single-letter abbreviations (e.g., Ala or A for alanine, Arg or R for arginine, etc.). Unless otherwise indicated, three-letter and single-letter abbreviations of amino acids refer to the L-isomeric form of the amino acid in question. The term “L-amino acid,” as used herein, refers to the “L” isomeric form of a peptide, and conversely the term “D-amino acid” refers to the “D” isomeric form of a peptide (e.g., (D)Asp or D-Asp; (D)Phe or D-Phe). Amino acid residues in the D isomeric form can be substituted for any L-amino acid residue, as long as the desired function is retained by the peptide. D-amino acids may be indicated as customary in lower case when referred to using single-letter abbreviations. For example, L-arginine can be represented as “Arg” or “R,” while D-arginine can be represented as “arg” or “r.” Similarly, L-lysine can be represented as “Lys” or “K,” while D-lysine can be represented as “lys” or “k.” Alternatively, a lower case “d” in front of an amino acid can be used to indicate that it is of the D isomeric form, for example D-lysine can be represented by dK.

In the case of less common or non-naturally occurring amino acids, unless they are referred to by their full name (e.g. sarcosine, ornithine, etc.), frequently employed three- or four-character codes are employed for residues thereof, including, Sar or Sarc (sarcosine, i.e. N-methylglycine), Aib (α-aminoisobutyric acid), Dab (2,4-diaminobutanoic acid), Dapa (2,3-diaminopropanoic acid), γ-Glu (γ-glutamic acid), Gaba (γ-aminobutanoic acid), β-Pro (pyrrolidine-3-carboxylic acid), and Abu (2-amino butyric acid).

Amino acids of the D-isomeric form may be located at any of the positions in the IL-23R inhibitors set forth herein (any of X1-X18 appearing in the molecule). In an aspect, amino acids of the D-isomeric form may be located only at any one or more of X3, X5, X6, X8, X13, and optionally one additional position. In other aspects, amino acids of the D-isomeric form may be located only at any one or more of X3, X8, X13, and optionally one additional position. In other aspects, amino acids of the D-isomeric form may be located only at any one or more of X8, X13 (e.g., X8 is dK(Ac) and X13 is dE), and optionally one additional position. In other aspects, amino acids of the D-isomeric form may be located only at X3, and optionally one additional position. In other aspects, amino acids of the D-isomeric form may be located only at X3, and optionally two or three additional positions. In other aspects, amino acids of the D-isomeric form may be located at only one or two of positions X1 to X18 appearing in the IL-23R inhibitors set forth herein. In other aspects, amino acids of the D-isomeric form may be located at only three or four of positions X1 to X18 appearing in the IL-23R inhibitors set forth herein. For example an IL-23R inhibitors set forth herein having only positions X3 to X15 present may have amino acids of the D-form present in 3 or four of those positions. In other aspects, amino acids of the D-isomeric form may be located at only five or six of positions X1 to X18 appearing in the IL-23R inhibitors set forth herein.

As is clear to the skilled artisan, the peptide sequences disclosed herein are shown proceeding from left to right, with the left end of the sequence being the N-terminus of the peptide and the right end of the sequence being the C-terminus of the peptide. Among sequences disclosed herein are sequences incorporating either an “—OH” moiety or an “—NH₂” moiety at the carboxy terminus (C-terminus) of the sequence. In such cases, and unless otherwise indicated, an “—OH” or an “—NH₂” moiety at the C-terminus of the sequence indicates a hydroxy group or an amino group, corresponding to the presence of a carboxylic acid (COOH) or an amido (CONH₂) group at the C-terminus, respectively. In each sequence of the invention, a C-terminal “—OH” moiety may be substituted for a C-terminal “—NH₂” moiety, and vice-versa

As used herein, the term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in forming a bridge for cyclization such as a thio ether bond and a disulfide bond for forming a cyclic portion of a peptide, for example as in the connection between the amino acid residue at position Z4 and amino acid residue at position Z9 in various embodiments discussed herein. Amino acids may comprise one or more post-translational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

As used herein, “Hydrophilic Amino Acid or Residue” refers to an amino acid or residue having a side chain exhibiting a hydrophobicity of less than zero according to the normalized consensus hydrophobicity scale of Eisenberg et al., 1984, J. Mol. Biol. 179:125-142. Examples of hydrophilic amino acids include but are not limited to L-Thr (T), L-Ser (S), L-His (H), L-Glu (E), L-Asn (N), L-Gln (Q), L-Asp (D), L-Lys (K) and L-Arg (R).

As used herein, “Acidic Amino Acid or Residue” refers to a hydrophilic amino acid or residue having a side chain exhibiting a pK value of less than about 6 when the amino acid is included in a peptide or polypeptide. Acidic amino acids typically have negatively charged side chains at physiological pH due to loss of a hydrogen ion. Examples of acidic amino acids include but are not limited to L-Glu (E) and L-Asp (D)

As used herein, “Basic Amino Acid or Residue” refers to a hydrophilic amino acid or residue having a side chain exhibiting a pK value of greater than about 6 when the amino acid is included in a peptide or polypeptide. Basic amino acids typically have positively charged side chains at physiological pH due to association with hydronium ion. Examples of basic amino acids include but are not limited to L-Arg (R) and L-Lys (K)

As used herein, “Polar Amino Acid or Residue” refers to a hydrophilic amino acid or residue having a side chain that is uncharged at physiological pH, but which has at least one bond in which the pair of electrons shared in common by two atoms is held more closely by one of the atoms. Examples of polar amino acids include but are not limited to L-Asn (N), L-Gln (Q), L-Ser (S) and L-Thr (T).

As used herein, “Hydrophobic Amino Acid or Residue” refers to an amino acid or residue having a side chain exhibiting a hydrophobicity of greater than zero according to the normalized consensus hydrophobicity scale of Eisenberg et al., 1984, J. Mol. Biol. 179:125-142. Examples of hydrophobic amino acids include but are not limited to L-Pro (P), L-Ile (I), L-Phe (F), L-Val (V), L-Leu (L), L-Trp (W), L-Met (M), L-Ala (A) and L-Tyr (Y).

As used herein, “Aromatic Amino Acid or Residue” refers to a hydrophilic or hydrophobic amino acid or residue having a side chain that includes at least one aromatic or heteroaromatic ring. Examples of aromatic amino acids include but are not limited to L-Phe (F), L-Tyr (Y), L-His (H), 2-Nal, substituted 2-Nal, AEF, substituted AEF, L-Trp (W), Trp or substituted Trp. Although owing to the pKa of its heteroaromatic nitrogen atom L-His (H) it is sometimes classified as a basic residue, herein histidine is classified as an aromatic residue as its side chain includes a heteroaromatic ring.

As used herein, “Non-polar Amino Acid or Residue” refers to a hydrophobic amino acid or residue having a side chain that is uncharged at physiological pH and which has bonds in which the pair of electrons shared in common by two atoms is generally held equally by each of the two atoms (i.e., the side chain is not polar). Examples of non-polar amino acids include but are not limited to L-Gly (G), L-Leu (L), L-Val (V), L-Ile (I), L-Met (M), L-Pro (P) and L-Ala (A).

As used herein, “Aliphatic Amino Acid or Residue” refers to a hydrophobic amino acid or residue having an aliphatic hydrocarbon side chain. Examples of aliphatic amino acids include but are not limited to L-Ala (A), L-Val (V), L-Leu (L) and L-Ile (I).

The amino acid L-Cys (C) is unusual in that it can form disulfide bridges with other L-Cys (C) amino acids or other sulfanyl- or sulfhydryl-containing amino acids. As used herein, “cysteine-like amino acids or residues” includes cysteine and other amino acids that contain sulfhydryl moieties that are available for formation of disulfide bridges. The ability of L-Cys (C) (and other amino acids with SH containing side chains) to exist in a peptide in either the reduced free SH or oxidized disulfide-bridged form affects whether L-Cys (C) contributes net hydrophobic or hydrophilic character to a peptide. While L-Cys (C) exhibits a hydrophobicity of 0.29 according to the normalized consensus scale of Eisenberg (Eisenberg et al., 1984, supra), it is to be understood that for purposes of the present disclosure L-Cys (C) is categorized into its own unique group. Examples of “cysteine-like amino acids or residues” include but are not limited to (R)-2-amino-3-mercapto-3-methylbutanoic acid (Pen) and L-homocysteine (hC).

The term “quaternary amine” takes its ordinary meaning in the art. For example, a quaternary amine is a substituent comprising one or more nitrogen atom(s) that is/are permanently positively changed. The permanent positive charge of the nitrogen atom(s) may be independent of the surrounding pH. One non-limiting example of a quaternary amine can be a moiety having four organic substituents on a nitrogen atom.

The term “masked amine” means a substituent comprising one or more nitrogen atom(s), wherein the one or more nitrogen atom(s) is/are substituted. For example, the one or more nitrogen atom(s) may be substituted with groups such as alkyl or acetyl, to form secondary amine(s), tertiary amine(s) or amides. In embodiments, a masked amine is a secondary amine. In embodiments, a masked amine is a tertiary amine. In embodiments, a masked amine is an amide.

The term “masked amide” means a substituent comprising one or more nitrogen atom(s), wherein the one or more nitrogen atom(s) is/are substituted with —C ═O, and at least one other substituent. For example, the one or more nitrogen atom(s) substituted with —C═O may be further substituted with groups such as alkyl or acetyl, to form secondary amide(s) or tertiary amides. In embodiments, a masked amide is a secondary amide. In some embodiments, a masked amide is a tertiary amide.

One of skill in the art will appreciate that certain amino acids and other chemical moieties are modified when bound to another molecule. For example, an amino acid side chain may be modified when it forms an intramolecular bridge with another amino acid side chain, e.g., one or more hydrogen may be removed or replaced by the bond.

A “compound of the invention”, an “inhibitor of the present disclosure”, an “IL-23R inhibitor of the present disclosure”, a “compound described herein”, and a “herein-described compound” include the novel compounds disclosed herein, for example the compounds of any of the Examples, including compounds of Formula (I) to (VI) such as those found in Table 1A, Table 1B, Table 1C, Table 1D, or Table 1E.

“Pharmaceutically effective amount” refers to an amount of a compound of the invention in a composition or combination thereof that provides the desired therapeutic or pharmaceutical result.

By “pharmaceutically acceptable” it is meant the carrier(s), diluent(s), salts, or excipient(s) must be compatible with the other components or ingredients of the compositions of the present invention, i.e., that which is useful, safe, non-toxic acceptable for pharmaceutical use. In accordance with the present invention pharmaceutically acceptable means approved or approvable as is listed in the U.S. Pharmacopoeia or other generally recognized pharmacopoeia for use in animals, and more particularly, in humans.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Absorption enhancer” refers to a component that improves or facilitates the mucosal absorption of a drug in the gastrointestinal tract, such as a permeation enhancer or intestinal permeation enhancer. As conventionally understood in the art, permeation enhancers (PEs) are agents aimed to improve oral delivery of therapeutic drugs with poor bioavailability. PEs are capable of increasing the paracellular and/or transcellular passage of drugs.

Pharmaceutical excipients that can increase permeation have been termed “absorption modifying excipients” (AMEs). AMEs may be used in oral compositions, for example, as wetting agents (sodium dodecyl sulfate), antioxidants (e.g. EDTA), and emulsifiers (e.g. macrogol glycerides), and may be specifically included in compositions as PEs to improve bioavailability. PEs can be categorized as to how they alter barrier integrity via paracellular or transcellular routes.

“Intestinal permeation enhancer (IPE)” refers to a component that improves the bioavailability of a component. Suitable representative IPEs for use in the present invention, include, but are not limited to, various surfactants, fatty acids, medium chain glycerides, steroidal detergents, acyl carnitine and alkanoylcholines, N-acetylated alpha-amino acids and N-acetylated non-alpha-amino acids, and chitosans, other mucoadhesive polymers and the like. For example, a suitable IPE for use in the present invention may be sodium caprate.

“Composition” or “Pharmaceutical Composition” as used herein is intended to encompass an invention or product comprising the specified active product ingredient (API), which may include pharmaceutically acceptable excipients, carriers or diluents as described herein, such as in specified amounts defined throughout the disclosure. Compositions or Pharmaceutical Compositions result from combination of specific components, such as specified ingredients in the specified amounts as described herein.

Compositions or pharmaceutical compositions of the present invention may be in different pharmaceutically acceptable forms, which may include, but are not limited to a liquid composition, a tablet or matrix composition, a capsule composition, etc. and the like. When the composition is a tablet composition, the tablet may include, but is not limited to different layers two or more different phases, including an internal phase and an external phase that can comprise a core. The tablet composition can also include, but is not limited to, one or more coatings.

“Solvate” as used herein, means a physical association of the compound of the present invention with one or more solvent molecules. This physical association involves varying degrees bonding, including hydrogen bonding. In certain instances, the solvate will be capable of isolation. The term “solvate” is intended to encompass both solution-phase and isolatable solvates. Non-limiting examples of suitable solvates include hydrates.

Provided are also pharmaceutically acceptable salts and tautomeric forms of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The IL-23R inhibitors of the present disclosure, or their pharmaceutically acceptable salts or solvates may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms of the IL-23R inhibitors of the present disclosure. Optically active (+) and (−), (R)- and (S)-, or (D)- and (L)- isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included. Where compounds are represented in their chiral form, it is understood that the aspect encompasses, but is not limited to, the specific diastereomerically or enantiomerically enriched form. Where chirality is not specified but is present, it is understood that the aspect is directed to either the specific diastereomerically or enantiomerically enriched form; or a racemic or scalemic mixture of such compound(s). As used herein, “scalemic mixture” is a mixture of stereoisomers enantiomers at a ratio other than 1:1.

Certain examples contain amino acids that are depicted or labelled as an (R*) or (S*). When (R*) or (S*) is used in the name of an amino acid or in the chemical representation of the amino acid, it is intended to convey that the amino acid is a pure single isomer at that stereocenter; however, absolute configuration of that stereocenter has not been established. Thus, a compound designated as (R*) refers to an amino acid that is a pure single isomer at that stereocenter with an absolute configuration of either (R) or (S), and a compound designated as (S*) refers to an amino acid that is a pure single isomer at that stereocenter with an absolute configuration of either (R) or (S). For example, ACHMF(S*):

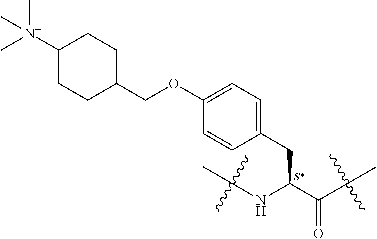 [SMILES: *N[C@@H](Cc1ccc(OCC2CCC([N+](C)(C)C)CC2)cc1)C(*)=O]

refers to an amino acid that is either:

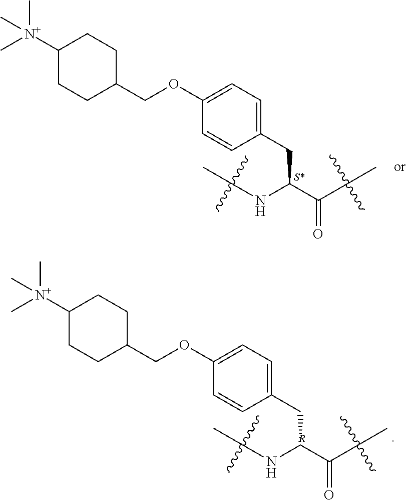

“Racemates” refers to a mixture of enantiomers. The mixture can include equal or unequal amounts of each enantiomer.

“Stereoisomer” and “stereoisomers” refer to compounds that differ in the chirality of one or more stereo centers. Stereoisomers include enantiomers and diastereomers.

The compounds may exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see, e.g., Chapter 4 of Advanced Organic Chemistry, 4th ed., J. March, John Wiley and Sons, New York, 1992).

“Tautomer” refers to alternate forms of a compound that differ in the position of a proton, such as enol-keto and imine-enamine tautomers, or the tautomeric forms of heteroaryl groups containing a ring atom attached to both a ring —NH— and a ring ═N— such as pyrazoles, imidazoles, benzimidazoles, triazoles, and tetrazoles.

Unless defined otherwise, all technical and scientific terms used herein have the same meaning as commonly or conventionally understood by one of ordinary skill in the art. In the chemical arts a dash at the front or end of a chemical group is a matter of convenience; chemical groups may be depicted with or without one or more dashes without losing their ordinary meaning. A wavy line drawn through a line in a structure indicates a point of attachment of a group. A dashed line indicates an optional bond. Unless chemically or structurally required, no directionality is indicated or implied by the order in which a chemical group is written or the point at which it is attached to the remainder of the molecule. For instance, the group “—SO₂CH₂—” is equivalent to “—CH₂SO₂—” and both may be connected in either direction. Similarly, an “arylalkyl” group, for example, may be attached to the remainder of the molecule at either an aryl or an alkyl portion of the group. A prefix such as “Cu ” or (Cᵤ-Cᵥ) indicates that the following group has from u to v carbon atoms. For example, “C₁-6alkyl” and “C₁-C₆ alkyl” both indicate that the alkyl group has from 1 to 6 carbon atoms.

“Treatment” or “treat” or “treating” as used herein refers to an approach for obtaining beneficial or desired results. For purposes of the present invention, beneficial or desired results include, but are not limited to, alleviation of a symptom and/or diminishment of the extent of a symptom and/or preventing a worsening of a symptom associated with a disease or condition. In one aspect, “treatment” or “treating” includes one or more of the following: (a) inhibiting the disease or condition (e.g., decreasing one or more symptoms resulting from the disease or condition, and/or diminishing the extent of the disease or condition); (b) slowing or arresting the development of one or more symptoms associated with the disease or condition (e.g., stabilizing the disease or condition, delaying the worsening or progression of the disease or condition); and (c) relieving the disease or condition, e.g., causing the regression of clinical symptoms, ameliorating the disease state, delaying the progression of the disease, increasing the quality of life, and/or prolonging survival.

“Therapeutically effective amount” or “effective amount” as used herein refers to an amount that is effective to elicit the desired biological or medical response, including the amount of a compound that, when administered to a subject for treating a disease is sufficient to effect such treatment for the disease. The effective amount will vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (e.g., additive or synergistic effects) of the compounds.

“Co-administration” as used herein refers to administration of unit dosages of the compounds disclosed herein before or after administration of unit dosages of one or more additional therapeutic agents, for example, administration of the compound disclosed herein within seconds, minutes, or hours of the administration of one or more additional therapeutic agents. For example, in some aspects, a unit dose of a compound of the invention is administered first, followed within seconds or minutes by administration of a unit dose of one or more additional therapeutic agents. Alternatively, in other aspects, a unit dose of one or more additional therapeutic agents is administered first, followed by administration of a unit dose of a compound of the invention within seconds or minutes. In some aspects, a unit dose of a compound of the invention is administered first, followed, after a period of hours (e.g., 1-12 hours), by administration of a unit dose of one or more additional therapeutic agents. In other aspects, a unit dose of one or more additional therapeutic agents is administered first, followed, after a period of hours (e.g., 1-12 hours), by administration of a unit dose of a compound of the invention. Co-administration of a compound disclosed herein with one or more additional therapeutic agents generally refers to simultaneous or sequential administration of a compound disclosed herein and one or more additional therapeutic agents, such that therapeutically effective amounts of each agent are present in the body of the patient.

Abbreviation, “(V/V)” refers to the phrase “volume for volume”, i.e., the proportion of a particular substance within a mixture, as measured by volume or a volume amount of a component of the composition disclosed herein relative to the total volume amount of the composition. Accordingly, the quantity is unit less and represents a volume percentage amount of a component relative to the total volume of the composition. For example, a 2% (V/V) solvent mixture can indicate 2 mL of one solvent is present in 100 mL of the solvent mixture.

Abbreviation, “(w/w)” refers to the phrase “weight for weight”, i.e., the proportion of a particular substance within a mixture, as measured by weight or mass or a weight amount of a component of the composition disclosed herein relative to the total weight amount of the composition. Accordingly, the quantity is unit less and represents a weight percentage amount of a component relative to the total weight of the composition. For example, a 2% (w/w) solution can indicate 2 grams of solute is dissolved in 100 grams of solution.

Systemic routes of administration as conventionally understood in the medicinal or pharmaceutical arts, refer to or are defined as a route of administration of drug, a pharmaceutical composition or formulation, or other substance into the circulatory system so that various body tissues and organs are exposed to the drug, formulation or other substance. As conventionally understood in the art, administration can take place orally (where drug or oral preparations are taken by mouth, and absorbed via the gastrointestinal tract), via enteral administration (absorption of the drug also occurs through the gastrointestinal tract) or parenteral administration (generally injection, infusion, or implantation, etc.

“Systemically active” peptide drug therapy as it relates to the present invention generally refers to treatment by means of a pharmaceutical composition comprising a peptide active ingredient, wherein said peptide resists immediate metabolism and/or excretion resulting in its exposure in various body tissues and organs, such as the cardiovascular, respiratory, gastrointestinal, nervous or immune systems.

Systemic drug activity in the present invention also refers to treatment using substances that travel through the bloodstream, reaching and affecting cells in various body tissues and organs. Systemic active drugs are transported to their site of action and work throughout the body to attack the physiological processes that cause inflammatory diseases.

“Bioavailability” refers to the extent and rate at which the active moiety (drug or metabolite) enters systemic circulation, thereby accessing the site of action. Bioavailability of a drug is impacted by the properties of the dosage form, which depend partly on its design and manufacture.

“Digestive tract tissue” as used herein refers to all the tissues that comprise the organs of the alimentary canal. For example only, and without limitation, “digestive tract tissue” includes tissues of the mouth, esophagus, stomach, small intestine, large intestine, duodenum, and anus.

As used herein, the term “natural polymer” takes its ordinary meaning in the art. For example, a polymer that can be found in living systems, such as plants, animals, bacteria, and fungi. A natural polymer may be a polypeptide, polysaccharide or polynucleotide. Subunits of natural polymers can be amino acids, monosaccharides or nucleotides. A natural polymer can vary in type (e.g., homopolymer or copolymer; random, alternating or block copolymer; linear or branched). A non-limiting example of a natural polymeric group is an amino acid sequence containing from about 10 to about 30 amino acids derived from (poly)peptides such as, natriuretic peptide precursor C, atrial natriuretic peptide, brain natriuretic peptide, serum albumin, IgG, histidine-rich glycoproteins, fibronectin, fibrinogen, zinc finger-containing polypeptides, osteocrin or fibroblast growth factor 2 (FGF2), or variants thereof with substitutions and/or deletions.

As used herein, the term “unnatural polymer” takes its ordinary meaning in the art. For example, a synthetic polymer that does not naturally occur in a living system, such as plants, animals, bacteria, and fungi. An unnatural polymer can vary in type (e.g., homopolymer or copolymer; random, alternating or block copolymer; linear or branched). A non-limiting example of an unnatural polymeric group is polyethylene glycol (PEG) (also called polyethylene oxide (PEO)).

As used herein, “hydrophilic polymer” takes its ordinary meaning in the art. For example, a polymer which dissolves in water. A hydrophilic polymer may comprise polar or charged functional groups that render the polymer soluble in water. A hydrophilic polymer may be a natural polymer or an unnatural polymer or a combination thereof. A hydrophilic polymer can vary in type (e.g., homopolymer or copolymer; random, alternating or block copolymer; linear or branched). A non-limiting example of a hydrophilic polymer includes polyethylene glycol (PEG).

### Chemical Definitions

Acyl: As used herein, the term “acyl” refers to R^{Z}—(C═O)—, wherein R^{Z} is, for example, any alkyl, alkenyl, alkynyl, heteroalkyl or heteroalkenyl.

Aliphatic: As used herein, the term aliphatic in the context of chemical substituents refers to a hydrocarbon and includes both saturated and unsaturated hydrocarbons. An aliphatic may be linear, branched, or cyclic. For example, C₁-C₁₀ aliphatics can include C₁-C₁₀ alkyls (e.g., linear or branched C₁-C₁₀ saturated alkyls), C₂-C₁₀ alkenyls (e.g., linear or branched C₄-C₁₀ dienyls, linear or branched C₆-C₁₀ trienyls, and the like), and C₂-C₁₀ alkynyls (e.g., linear or branched C₂-C₁₀ alkynyls). C₁-C₁₀ aliphatics can include C₃-C₁₀ cyclic aliphatics (e.g., C₃-C₁₀ cycloalkyls, C₄-C₁₀ cycloalkenyls, or C₅-C₁₀ cycloalkynyls). In certain embodiments, the aliphatic may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. An aliphatic group is unsubstituted or substituted with one or more substituent groups as described herein. For example, an aliphatic may be substituted with one or more (e.g., 1, 2, 3, 4, 5, or 6 independently selected substituents) of halogen, —COR^{aa}, —CO₂H, —CONH₂, —CO₂R″, —CN, —OH, —OR^{aa}, —OCOR^{aa}, —OCO₂R″, —NH₂, —NHR″, —N(R^{aa})₂, —SR″ or —SO₂R″, wherein each instance of R″ independently is C₁-C₁₀ aliphatic (e.g., C₁-C₁₀ alkyl, C₁-C₈ alkyl, C₁-C₆ alkyl, or C₁-C₃ alkyl). In embodiments, R″ independently is an unsubstituted alkyl (e.g., unsubstituted C₁-C₁₀ alkyl, C₁-C₈ alkyl, C₁-C₆ alkyl, or C₁-C₃ alkyl). In embodiments, R″ independently is unsubstituted C₁-C₃ alkyl. In embodiments, the aliphatic is unsubstituted. In embodiments, the aliphatic does not include any heteroatoms.

Alkyl: As used herein, the term “alkyl” refers to a radical of an acyclic linear and branched hydrocarbon groups, e.g. “C₁-C₆ alkyl” refers to alkyl groups having 1-6 carbons. An alkyl group may be linear or branched. Examples of alkyl groups include, but are not limited to, methyl, ethyl, n-propyl, isopropyl, butyl, isobutyl, sec-butyl, tert-butyl, pentyl, isopentyl tert-pentylhexyl, isohexyl, etc. The term “lower alkyl” means an alkyl group straight chain or branched alkyl having 1 to 6 carbon atoms. Other alkyl groups will be readily apparent to those of skill in the art given the benefit of the present disclosure. An alkyl group may be unsubstituted or substituted with one or more substituent groups as described herein. For example, an alkyl group may be substituted with one or more (e.g., 1, 2, 3, 4, 5, or 6 independently selected substituents) of halogen, —COR^{aa}, —CO₂H, —CONH₂, —CO₂R″, —CN, —OH, —OR^{aa}, —OCOR^{aa}, —OCO₂R″, —NH₂, —NHR″, —N(R^{aa})₂, —SR″ or —SO₂R″, wherein each instance of R″ independently is C₁-C₁₀ aliphatic (e.g., C₁-C₁₀ alkyl, C₁-C₈ alkyl, C₁-C₆ alkyl, or C₁-C₃ alkyl). In embodiments, R″ independently is an unsubstituted alkyl (e.g., unsubstituted C₁-C₁₀ alkyl, C₁-C₈ alkyl, C₁-C₆ alkyl, or C₁-C₃ alkyl). In embodiments, R″ independently is unsubstituted C₁-C₃ alkyl. In embodiments, the alkyl is substituted (e.g., with 1, 2, 3, 4, 5, or 6 substituent groups as described herein). In embodiments, an alkyl group is substituted with a —OH group and may also be referred to herein as a “hydroxyalkyl” group, where the prefix denotes the —OH group and “alkyl” is as described herein.

For example, “alkyl” refers to a radical of a straight-chain or branched saturated hydrocarbon group having from 1 to 10 carbon atoms (“C₁-C₁₀ alkyl”). In some embodiments, an alkyl group has 1 to 9 carbon atoms (“C₁-C₉ alkyl”). In some embodiments, an alkyl group has 1 to 8 carbon atoms (“C₁-C₈ alkyl”). In some embodiments, an alkyl group has 1 to 7 carbon atoms (“C₁-C₇ alkyl”). In some embodiments, an alkyl group has 1 to 6 carbon atoms (“C₁-C₆ alkyl”). In some embodiments, an alkyl group has 1 to 5 carbon atoms (“C₁-C₅ alkyl”). In some embodiments, an alkyl group has 1 to 4 carbon atoms (“C₁-C₄ alkyl”). In some embodiments, an alkyl group has 1 to 3 carbon atoms (“C₁-C₃ alkyl”). In some embodiments, an alkyl group has 1 to 2 carbon atoms (“C₁-C₂ alkyl”). In some embodiments, an alkyl group has 1 carbon atom (“C₁ alkyl”). In some embodiments, an alkyl group has 2 to 6 carbon atoms (“C₂-C₆ alkyl”). Examples of C₁-C₆ alkyl groups include, without limitation, methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently unsubstituted (an “unsubstituted alkyl”) or substituted (a “substituted alkyl”) with one or more substituents. In certain embodiments, the alkyl group is an unsubstituted C₁-C₆ alkyl. In certain embodiments, the alkyl group is a substituted C₁-C₆ alkyl.

Affixing the suffix “-ene” to a group indicates the group is a divalent moiety, e.g., arylene is the divalent moiety of aryl, and heteroarylene is the divalent moiety of heteroaryl.

Alkylene: The term “alkylene,” as used herein, represents a saturated divalent straight or branched chain hydrocarbon group and is exemplified by methylene, ethylene, isopropylene and the like. Likewise, the term “alkenylene” as used herein represents an unsaturated divalent straight or branched chain hydrocarbon group having one or more unsaturated carbon-carbon double bonds that may occur in any stable point along the chain, and the term “alkynylene” herein represents an unsaturated divalent straight or branched chain hydrocarbon group having one or more unsaturated carbon-carbon triple bonds that may occur in any stable point along the chain. In certain embodiments, an alkylene, alkenylene, or alkynylene group may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. For example, an alkylene, alkenylene, or alkynylene may be substituted with one or more (e.g., 1, 2, 3, 4, 5, or 6 independently selected substituents) of halogen, —COR^{aa}, —CO₂H, —CONH₂, —CO₂R″, —CN, —OH, —OR^{aa}, —OCOR^{aa}, —OCO₂R″, —NH₂, —NHR″, —N(R^{aa})₂, —SR″ or —SO₂R″, wherein each instance of R″ independently is C₁-C₁₀ aliphatic (e.g., C₁-C₁₀ alkyl, C₁-C₈ alkyl, C₁-C₆ alkyl, or C₁-C₃ alkyl). In embodiments, R″ independently is an unsubstituted alkyl (e.g., unsubstituted C₁-C₁₀ alkyl, C₁-C₈ alkyl, C₁-C₆ alkyl, or C₁-C₃ alkyl). In embodiments, R″ independently is unsubstituted C₁-C₃ alkyl. In certain embodiments, an alkylene, alkenylene, or alkynylene is unsubstituted. In certain embodiments, an alkylene, alkenylene, or alkynylene does not include any heteroatoms.

Alkenyl: As used herein, “alkenyl” refers to a radical of any linear or branched hydrocarbon chains having one or more unsaturated carbon-carbon double bonds that may occur in any stable point along the chain, e.g. “C₂-C₁₀ alkenyl” refers to an alkenyl group having 2-10 carbons. For example, an alkenyl group includes prop-2-enyl, but-2-enyl, but-3-enyl, 2-methylprop-2-enyl, hex-2-enyl, hex-5-enyl, 2,3-dimethylbut-2-enyl, and the like. In embodiments, the alkenyl comprises 1, 2, or 3 carbon-carbon double bond. In embodiments, the alkenyl comprises a single carbon-carbon double bond. In embodiments, multiple double bonds (e.g., 2 or 3) are conjugated. An alkenyl group may be unsubstituted or substituted with one or more substituent groups as described herein. For example, an alkenyl group may be substituted with one or more (e.g., 1, 2, 3, 4, 5, or 6 independently selected substituents) of halogen, —COR^{aa}, —CO₂H, —CONH₂, —CO₂R″, —CN, —OH, —OR^{aa}, —OCOR^{aa}, —OCO₂R″, —NH₂, —NHR″, —N(R^{aa})₂, —SR″ or —SO₂R″, wherein each instance of R″ independently is C₁-C₁₀ aliphatic (e.g., C₁-C₁₀ alkyl, C₁-C₈ alkyl, C₁-C₆ alkyl, or C₁-C₃ alkyl). In embodiments, R″ independently is an unsubstituted alkyl (e.g., unsubstituted C₁-C₁₀ alkyl, C₁-C₈ alkyl, C₁-C₆ alkyl, or C₁-C₃ alkyl). In embodiments, R″ independently is unsubstituted C₁-C₃ alkyl. In embodiments, the alkenyl is unsubstituted. In embodiments, the alkenyl is substituted (e.g., with 1, 2, 3, 4, 5, or 6 substituent groups as described herein). In embodiments, an alkenyl group is substituted with a —OH group and may also be referred to herein as a “hydroxyalkenyl” group, where the prefix denotes the —OH group and “alkenyl” is as described herein.

For example, “alkenyl” refers to a radical of a straight-chain or branched hydrocarbon group having from 2 to 10 carbon atoms and one or more carbon-carbon double bonds (e.g., 1, 2, 3, or 4 double bonds) (“C₂-C₁₀ alkenyl”). In some embodiments, an alkenyl group has 2 to 9 carbon atoms (“C₂-C₉ alkenyl”). In some embodiments, an alkenyl group has 2 to 8 carbon atoms (“C₂-C₈ alkenyl”). In some embodiments, an alkenyl group has 2 to 7 carbon atoms (“C₂-C₇ alkenyl”). In some embodiments, an alkenyl group has 2 to 6 carbon atoms (“C₂-C₆ alkenyl”). In some embodiments, an alkenyl group has 2 to 5 carbon atoms (“C₂-C₅ alkenyl”). In some embodiments, an alkenyl group has 2 to 4 carbon atoms (“C₂-C₄ alkenyl”). In some embodiments, an alkenyl group has 2 to 3 carbon atoms (“C₂-C₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include, without limitation, ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂-C₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂-C₁₀ alkenyl. In certain embodiments, the alkenyl group is a substituted C₂-C₁₀ alkenyl.

Alkynyl: As used herein, “alkynyl” refers to a radical of any hydrocarbon chain of either linear or branched configuration, having one or more carbon-carbon triple bonds occurring in any stable point along the chain, e.g., “C₂-C₁₀ alkynyl”, refers to an alkynyl group having 2-10 carbons. Examples of an alkynyl group include prop-2-ynyl, but-2-ynyl, but-3-ynyl, pent-2-ynyl, 3-methylpent-4-ynyl, hex-2-ynyl, hex-5-ynyl, etc. In embodiments, an alkynyl comprises one carbon-carbon triple bond. An alkynyl group may be unsubstituted or substituted with one or more substituent groups as described herein. For example, an alkynyl group may be substituted with one or more (e.g., 1, 2, 3, 4, 5, or 6 independently selected substituents) of halogen, —COR^{aa}, —CO₂H, —CONH₂, —CO₂R″, —CN, —OH, —OR^{aa}, —OCOR^{aa}, —OCO₂R″, —NH₂, —NHR″, —N(R^{aa})₂, —SR″ or —SO₂R″, wherein each instance of R″ independently is C₁-C₁₀ aliphatic (e.g., C₁-C₁₀ alkyl, C₁-C₈ alkyl, C₁-C₆ alkyl, or C₁-C₃ alkyl). In embodiments, R″ independently is an unsubstituted alkyl (e.g., unsubstituted C₁-C₁₀ alkyl, C₁-C₈ alkyl, C₁-C₆ alkyl, or C₁-C₃ alkyl). In embodiments, R″ independently is unsubstituted C₁-C₃ alkyl. In embodiments, the alkynyl is unsubstituted. In embodiments, the alkynyl is substituted (e.g., with 1, 2, 3, 4, 5, or 6 substituent groups as described herein).

For example, “alkynyl” refers to a radical of a straight-chain or branched hydrocarbon group having from 2 to 10 carbon atoms and one or more carbon-carbon triple bonds (e.g., 1, 2, 3, or 4 triple bonds) and optionally one or more double bonds (e.g., 1, 2, 3, or 4 double bonds) (“C₂-C₁₀ alkynyl”). An alkynyl group that has one or more triple bonds and one or more double bonds is also referred to as an “ene-yne”. In some embodiments, an alkynyl group has 2 to 9 carbon atoms (“C₂-C₉ alkynyl”). In some embodiments, an alkynyl group has 2 to 8 carbon atoms (“C₂-C₈ alkynyl”). In some embodiments, an alkynyl group has 2 to 7 carbon atoms (“C₂-C₇ alkynyl”). In some embodiments, an alkynyl group has 2 to 6 carbon atoms (“C₂-C₆ alkynyl”). In some embodiments, an alkynyl group has 2 to 5 carbon atoms (“C₂-C₅ alkynyl”). In some embodiments, an alkynyl group has 2 to 4 carbon atoms (“C₂-C₄ alkynyl”). In some embodiments, an alkynyl group has 2 to 3 carbon atoms (“C₂-C₃ alkynyl”). In some embodiments, an alkynyl group has 2 carbon atoms (“C₂ alkynyl”). The one or more carbon-- triple bonds can be internal (such as in 2-butynyl) or terminal (such as in 1-butynyl). Examples of C₂-C₄ alkynyl groups include, without limitation, ethynyl (C₂), 1-propynyl (C₃), 2-propynyl (C₃), 1-butynyl (C₄), 2-butynyl (C₄), and the like. Examples of C₂-C₆ alkenyl groups include the aforementioned C₂-C₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂-C₁₀ alkynyl. In certain embodiments, the alkynyl group is a substituted C₂-C₁₀ alkynyl.

Aryl: The term “aryl” used alone or as part of a larger moiety as in “aralkyl,” refers to a monocyclic, bicyclic, or tricyclic carbocyclic ring system having a total of six to fourteen ring members, wherein said ring system has a single point of attachment to the rest of the molecule, at least one ring in the system is aromatic and wherein each ring in the system contains 4 to 7 ring members. In embodiments, an aryl group has 6 ring carbon atoms (“C₆ aryl,” e.g., phenyl). In some embodiments, an aryl group has 10 ring carbon atoms (“C₁₀ aryl,” e.g., naphthyl such as 1-naphthyl and 2-naphthyl). In some embodiments, an aryl group has 14 ring carbon atoms (“C₁₄ aryl,” e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Exemplary aryls include phenyl, naphthyl, and anthracene.

As used herein, “aryl” also refers to a radical of a monocyclic or polycyclic (e.g., bicyclic or tricyclic) 4n+2 aromatic ring system (e.g., having 6, 10, or 14 R electrons shared in a cyclic array) having 6-14 ring carbon atoms and zero heteroatoms provided in the aromatic ring system (“C₆-C₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). Unless otherwise specified, each instance of an aryl group is independently unsubstituted (an “unsubstituted aryl”) or substituted (a “substituted aryl”) with one or more substituents. In certain embodiments, the aryl group is an unsubstituted C₆-C₁₄ aryl. In certain embodiments, the aryl group is a substituted C₆-C₁₄ aryl.

Arylene: The term “arylene” as used herein refers to an aryl group that is divalent (that is, having two points of attachment to the molecule). Exemplary arylenes include phenylene (e.g., unsubstituted phenylene or substituted phenylene).

Carbocyclyl: As used herein, “carbocyclyl” or “carbocyclic” refers to a radical of a non-aromatic cyclic hydrocarbon group having from 3 to 10 ring carbon atoms (“C₃-C₁₀ carbocyclyl”) and zero heteroatoms in the non-aromatic ring system. In some embodiments, a carbocyclyl group has 3 to 8 ring carbon atoms (“C₃-C₈ carbocyclyl”). In some embodiments, a carbocyclyl group has 3 to 7 ring carbon atoms (“C₃-C₇ carbocyclyl”). In some embodiments, a carbocyclyl group has 3 to 6 ring carbon atoms (“C₃-C₆ carbocyclyl”). In some embodiments, a carbocyclyl group has 4 to 6 ring carbon atoms (“C₄-C₆ carbocyclyl”). In some embodiments, a carbocyclyl group has 5 to 6 ring carbon atoms (“C₅-C₆ carbocyclyl”). In some embodiments, a carbocyclyl group has 5 to 10 ring carbon atoms (“C₅-C₁₀ carbocyclyl”). Exemplary C₃-C₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ carbocyclyl groups include, without limitation, the aforementioned C₃-C₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃-C₁₀ carbocyclyl groups include, without limitation, the aforementioned C₃-C₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃-C₁₀ carbocyclyl. In certain embodiments, the carbocyclyl group is a substituted C₃-C₁₀ carbocyclyl.

In some embodiments, “carbocyclyl” or “carbocyclic” is referred to as a “cycloalkyl”, i.e., a monocyclic, saturated carbocyclyl group having from 3 to 10 ring carbon atoms (“C₃-C₁0 cycloalkyl”). In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆, cycloalkyl”). In some embodiments, a cycloalkyl group has 4 to 6 ring carbon atoms (“C₄-C₆ cycloalkyl”). In some embodiments, a cycloalkyl group has 5 to 6 ring carbon atoms (“C₅-C₆ cycloalkyl”). In some embodiments, a cycloalkyl group has 5 to 10 ring carbon atoms (“C₅-C₁₀ cycloalkyl”). Examples of C₅-C₆ cycloalkyl groups include cyclopentyl (C₅) and cyclohexyl (C₅). Examples of C₃-C₆ cycloalkyl groups include the aforementioned C₅-C₆ cycloalkyl groups as well as cyclopropyl (C₃) and cyclobutyl (C₄). Examples of C₃-C₈ cycloalkyl groups include the aforementioned C₃-C₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃-C₆ cycloalkyl. In certain embodiments, the cycloalkyl group is a substituted C₃-C₆ cycloalkyl.

Halogen: As used herein, the term “halogen” means fluorine, chlorine, bromine, or iodine.

Heteroalkyl: The term “heteroalkyl” refers to a radical of a branched or unbranched alkyl, alkenyl, or alkynyl group having carbon atoms in addition to heteroatoms independently selected from the group consisting of N, O, S, and P. For example, “heteroalkyl” can mean a branched or unbranched alkyl, alkenyl, or alkynyl group having from 1 to 8 carbon atoms in addition to 1, 2, 3 or 4 heteroatoms independently selected from the group consisting of N, O, S, and P. For example, the term “heteroalkyl” refers to a radical of a branched or unbranched alkyl, alkenyl, or alkynyl group having from 1 to 40 carbon atoms in addition to heteroatoms independently selected from the group consisting of N, O, S, and P (“C₁-C₄₀ heteroalkyl”). In an embodiment, the term “heteroalkyl” refers to a radical of a branched or unbranched alkyl group having from 1 to 40 carbon atoms in addition to heteroatoms independently selected from the group consisting of N, O, S, and P (“C₁-C₄₀ heteroalkyl”). In an embodiment, a “heteroalkyl” can comprise about 1 to about 7 heteroatoms, independently selected from the group consisting of N, O, S, and P, for every 10 carbons in the “heteroalkyl”. In an embodiment, a “heteroalkyl” can comprise about 1 to about 5 heteroatoms, independently selected from the group consisting of N, O, S, and P, for every 10 carbons in the “heteroalkyl”. In an embodiment, the heteroatoms are independently selected from the group consisting of N, O or S. In an embodiment, the heteroatoms are independently selected from the group consisting of N or O. Heteroalkyls include tertiary amines, secondary amines, ethers, thioethers, amides, thioamides, carbamates, thiocarbamates, hydrazones, imines, phosphodiesters, phosphoramidates, sulfonamides, and disulfides. A heteroalkyl group may optionally include monocyclic, bicyclic, or tricyclic rings, in which each ring desirably has three to eight members, for example a heteroalkyl group may optionally include one or more triazole ring(s). Examples of heteroalkyls include polyethers, such as methoxymethyl and ethoxyethyl, and hydrophilic polymers, such as polyethylene glycol (PEG).

Heteroalkylene: The term “heteroalkylene,” as used herein, represents a divalent form of a heteroalkyl group as described herein.

Heteroaryl: The term “heteroaryl,” as used herein, is fully unsaturated heteroatom-containing ring wherein at least one ring atom is a heteroatom such as, but not limited to, nitrogen and oxygen.

As used herein, “heteroaryl” also refers to a radical of a 5-14 membered monocyclic or polycyclic (e.g., bicyclic or tricyclic) 4n+2 aromatic ring system (e.g., having 6, 10, or 14 πL electrons shared in a cyclic array) having ring carbon atoms and 1 or more (e.g., 1, 2, 3, or 4 ring heteroatoms) ring heteroatoms provided in the aromatic ring system, wherein each heteroatom is independently selected from oxygen, sulfur, nitrogen, boron, silicon, and phosphorus (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-10 membered heteroaryl”). In some embodiments, a heteroaryl group is a 5-8 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-8 membered heteroaryl”). In some embodiments, a heteroaryl group is a 5-6 membered aromatic ring system having ring carbon atoms and 1 or more (e.g., 1, 2, 3, or 4) ring heteroatoms provided in the aromatic ring system, wherein each heteroatom is independently selected from oxygen, sulfur, nitrogen, boron, silicon, and phosphorus (“5-6 membered heteroaryl”). In some embodiments, the 5-6 membered heteroaryl has 1 or more (e.g., 1, 2, or 3) ring heteroatoms selected from oxygen, sulfur, nitrogen, boron, silicon, and phosphorus. In some embodiments, the 5-6 membered heteroaryl has 1 or 2 ring heteroatoms selected from oxygen, sulfur, nitrogen, boron, silicon, and phosphorus. In some embodiments, the 5-6 membered heteroaryl has 1 ring heteroatom selected from oxygen, sulfur, nitrogen, boron, silicon, and phosphorus. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

As used herein, “heterocyclyl” or “heterocyclic” refers to a radical of a 3- to 14-membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)). and can be saturated or can contain one or more carbon-carbon double or triple bonds.

Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-10 membered heterocyclyl”). In some embodiments, a heterocyclyl group is a 5-8 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-8 membered heterocyclyl”). In some embodiments, a heterocyclyl group is a 5-6 membered non-aromatic ring system having ring carbon atoms and 1 or more (e.g., 1, 2, 3, or 4) ring heteroatoms, wherein each heteroatom is independently selected from oxygen, sulfur, nitrogen, boron, silicon, and phosphorus (“5-6 membered heterocyclyl”). In some embodiments, the 5-6 membered heterocyclyl has 1 or more (e.g., 1, 2, or 3) ring heteroatoms selected from oxygen, sulfur, nitrogen, boron, silicon, and phosphorus. In some embodiments, the 5-6 membered heterocyclyl has 1 or 2 ring heteroatoms selected from oxygen, sulfur, nitrogen, boron, silicon, and phosphorus. In some embodiments, the 5-6 membered heterocyclyl has 1 ring heteroatom selected from oxygen, sulfur, nitrogen, boron, silicon, and phosphorus.

Exemplary 3-membered heterocyclyl groups containing 1 heteroatom include, without limitation, azirdinyl, oxiranyl, thiorenyl. Exemplary 4-membered heterocyclyl groups containing 1 heteroatom include, without limitation, azetidinyl, oxetanyl and thietanyl.

Exemplary 5-membered heterocyclyl groups containing 1 heteroatom include, without limitation. tetrahydrofuranyl, dihydrofuranyl, tetrahydrothiophenyl, dihydrothiophenyl, pyrrolidinyl, dihydropyrrolyl and pyrrolyl-2,5-dione. Exemplary 5- membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, dioxanyl. Exemplary 6-membered heterocyclyl groups containing 2 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno [3,2- b]pyridinyl, 1,2,3,4-tetrahydro-1,6-naphthyridinyl, and the like.

Heterocycloalkyl: The term “heterocycloalkyl,” as used herein, is a non-aromatic ring wherein at least one atom is a heteroatom such as, but not limited to, nitrogen, oxygen, sulfur, or phosphorus, and the remaining atoms are carbon. In some embodiments, a heterocycloalkyl group has from 3 to 10 ring carbon atoms (“C₃-C₁₀ heterocycloalkyl”). In some embodiments, a heterocycloalkyl group has 3 to 8 ring carbon atoms (“C₃-C₈ heterocycloalkyl”). In some embodiments, a heterocycloalkyl group has 3 to 6 ring carbon atoms (“C₃-C₆, heterocycloalkyl”). In some embodiments, a heterocycloalkyl group has 4 to 6 ring carbon atoms (“C₄-C₆ heterocycloalkyl”). In some embodiments, a heterocycloalkyl group has 5 to 6 ring carbon atoms (“C₅-C₆ heterocycloalkyl”). In some embodiments, a heterocycloalkyl group has 5 to 10 ring carbon atoms (“C₅-C₁₀ heterocycloalkyl”). Examples of C₃-C₆ heterocycloalkyl groups include tetrahydropyranyl (C₅) and piperazinyl (C₄). Unless otherwise specified, each instance of a heterocycloalkyl group is independently unsubstituted (an “unsubstituted heterocycloalkyl”) or substituted (a “substituted heterocycloalkyl”) with one or more substituents. In certain embodiments, the heterocycloalkyl group is an unsubstituted C₃-C₆ heterocycloalkyl. In certain embodiments, the heterocycloalkyl group is a substituted C₃-C₆ heterocycloalkyl.

As understood from the above, alkyl, heteroalkyl, alkenyl, alkynyl, acyl, carbocyclyl, cycloalkyl, heterocyclyl, heterocycloalkyl, aryl, and heteroaryl groups, as defined herein, are, in certain embodiments, optionally substituted. Optionally substituted refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or ‘unsubstituted” heteroalkynyl, “substituted” or “unsubstituted” carbocyclyl, “substituted” or “unsubstituted” cycloalkyl, “substituted” or “unsubstituted” heterocyclyl, “substituted” or “unsubstituted” heterocycloalkyl, “substituted” or “unsubstituted” aryl or “substituted” or “unsubstituted” heteroaryl group. In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂, —SO₃H, —OH, —OR^{aa}, —ON(R^{bb})₂, —N(R^{bb})₂, —N(R^{bb})₃+X, —N(OR^{cc})R^{bb}, —SeH, —SeR^{aa}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}—S(═O)R^{aa}, —OS(═O)R^{aa}, —Si(R^{aa})₃—OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, - P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, - P(R^{cc})₃, —OP(R^{cc})₂, —OP(R^{cc})₃, —B(R^{aa})₂, —B(OR^{cc})₂, —BR^{aa}(OR^{cc}), C₁-C₁₀ alkyl, C₂-C₁₀ alkenyl, C₂-C₁₀ alkynyl, C₃-C₁₄ carbocyclyl, 3-14 membered heterocyclyl, C₆-C₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, ═NNR^{bb}C(═O)R—, ═NNR^{bb}C(═O)OR^{aa}, ═NNR^{bb}S(═O)₂R^{aa}, ═NR^{bb}, or ═NOR^{cc}; each instance of R^{aa} is, independently, selected from C₁-C₁₀ alkyl, C₂-C₁₀ alkenyl, C₂-C₁₀ alkynyl, C₃-C₁₀ carbocyclyl, 3-14 membered heterocyclyl, C₆-C₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd}groups;
each instance of R^{bb} is, independently, selected from hydrogen, —OH, —OR^{aa}, - N(R^{cc})₂, —CN, —C(═O)R^{aa}, —C(═O)N(R^{cc})₂, —CO₂R^{aa}, —SO₂R^{aa}, —C(═NR^{cc})OR^{aa}, - C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, - C(═S)SR^{cc}, —P(═O)₂R^{aa}, —P(═O)(R^{aa})₂, —P(═O)₂N(R^{cc})₂, —P(═O)(NR^{cc})₂, C₁-C₁₀ alkyl, C₂-C₁₀ alkenyl, C₂-C₁₀ alkynyl, C₃-C₁₀ carbocyclyl, 3-14 membered heterocyclyl, C₆-C₁₄ aryl, and 5-14 membered heteroaryl, or two R^{bb} groups, together with the heteroatom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁-C₁₀ alkyl, C₂-C₁₀ alkenyl, C₂-C₁₀ alkynyl, C₃-C₁₀ carbocyclyl, 3-14 membered heterocyclyl, C₆-C₁₄ aryl, and 5-14 membered heteroaryl, or two R^{cc} groups, together with the heteroatom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃+X⁻, —N(OR^{ee})R^{ee}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, - OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, - OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, - P(═O)(R^{ee})₂—OP(═O)(R^{ee})₂, —OP(═O)(OR^{ee})₂, C₁-C₁₀ alkyl, C₂-C₁₀ alkenyl, C₂-C₁₀ alkynyl, C₃-C₁₀ carbocyclyl, 3-10 membered heterocyclyl, C₆-C₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from C₁-C₁₀ alkyl, C₂-C₁₀ alkenyl, C₂-C₁₀ alkynyl, C₃-C₁₀ carbocyclyl, C₆-C₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁-C₁₀ alkyl, C₂-C₁₀ alkenyl, C₂-C₁₀ alkynyl, C₃-C₁₀ carbocyclyl, 3-10 membered heterocyclyl, C₆-C₁₀ aryl and 5-10 membered heteroaryl, or two R^{f} groups, together with the heteroatom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁-C₁₀ alkyl, —ON(C₁-C₁₀ alkyl)₂, —N(C₁-C₁₀ alkyl)₂, —N(C₁-C₁₀ alkyl)₃+X⁻, —NH(C₁-C₁₀ alkyl)₂+X⁻, —NH₂(C₁-C₁₀ alkyl)+X⁻, —NH₃+X⁻, —N(OC₁-C₁₀ alkyl)(C₁-C₁₀ alkyl), —N(OH)(C₁-C₁₀ alkyl), —NH(OH), —SH, —S, C₁-C₁₀ alkyl, —SS(C₁-C₁₀ alkyl), —C(═O)(C₁-C₁0 alkyl), —CO₂H, —CO₂(C₁-C₁₀ alkyl), —OC(═O)(C₁-C₁₀ alkyl), —OCO₂(C₁-C₁₀ alkyl), —C(═O)NH₂, —C(═O)N(C₁-C₁₀ alkyl)₂, —OC(═O)NH(C₁-C₁₀ alkyl), —NHC(═O)(C₁-C₁₀ alkyl), —N(C₁-C₁₀ alkyl)C(═O)(C₁-C₁₀ alkyl), —NHCO₂(C₁-C₁₀ alkyl), —NHC(═O)N(C₁-C₁₀ alkyl)₂, —NHC(═O)NH(C₁-C₁₀ alkyl), —NHC(═O)NH₂, —C(═NH)O(C₁-C₁₀ alkyl), —OC(═NH)(C₁-C₁₀ alkyl), —OC(═NH)OC₁-C₁₀ alkyl, - C(═NH)N(C₁-C₁₀ alkyl)₂, —C(═NH)NH(C₁-C₁₀ alkyl), —C(═NH)NH₂, —OC(═NH)N(C₁-C₁₀alkyl)₂, —OC(NH)NH(C₁-C₁₀ alkyl), —OC(NH)NH₂, —NHC(NH)N(C₁-C₁₀ alkyl)₂, —NHC(═NH)NH₂, —NHSO₂(C₁-C₁₀ alkyl), —SO₂N(C₁-C₁₀ alkyl)₂, —SO₂NH(C₁-C₁₀ alkyl), —SO₂NH₂, —SO₂(C₁-C₁₀ alkyl), —SO₂(C₁-C₁₀ alkyl), —OSO₂(C₁-C₆ alkyl), —SO(C₁-C₆ alkyl), —Si(C₁-C₁₀ alkyl)₃, —OSi(C₁-C₆ alkyl)₃, —C(═S)N(C₁-C₁₀ alkyl)₂, C(═S)NH(C₁-C₁₀ alkyl), C(═S)NH₂, —C(═O)S(C₁-C₆ alkyl), —C(═S)S(C₁-C₆ alkyl), —SC(═S)S(C₁-C₆ alkyl), —P(═O)₂(C₁-C₁₀ alkyl), —P(═O)(C₁-C₁₀ alkyl)₂, —OP(═O)(C₁-C₁₀ alkyl)₂, —OP(═O)(OC₁-C₁₀ alkyl)₂, C₁-C₁₀ alkyl, C₂-C₁₀ alkenyl, C₂-C₁₀ alkynyl, C₃-C₁₀ carbocyclyl, C₆-C₁₀ aryl, 3-10 membered heterocyclyl, 5-10 membered heteroaryl; or two geminal R⁹⁹ substituents can be joined to form ═O or ═S; wherein X—is a counterion.

As used herein, the term “halo” or “halogen” refers to fluorine (fluoro, —F), chlorine (chloro, —C₁), bromine (bromo, —Br), or iodine (iodo, —I).

As used herein, a “counterion” is a negatively charged group associated with a positively charged quarternary amine in order to maintain electronic neutrality. Exemplary counterions include halide ions (e.g., F⁻, C₁⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{o})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —CH₂(CO₂H), —P(═O)₂R^{aa}, —P(═O)(R^{aa})₂, —P(═O)₂N(R^{cc})₂, —P(═O)(NR^{cc})₂, C₁-C₁₀ alkyl, C₂-C₁₀ alkenyl, C₂-C₁₀ alkynyl, C₃-C₁₀ carbocyclyl, 3-14 membered heterocyclyl, C₆-C₁₄ aryl, and 5-14 membered heteroaryl, or two R^{cc} groups, together with the N atom to which they are attached, form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is a nitrogen protecting group (also referred to as an amino protecting group). Nitrogen protecting groups are well known in the art and include those described in detail in Protecting Groups in Organic Synthesis, T. W. Greene and P. G. M. Wuts, 3rd edition, John Wiley & Sons, 1999, incorporated herein by reference

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamanty1)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′-and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1(3,5-dimethoxyphenyl)ethyl carbamate, 1-methyl-1-(p-phenylazophenyl)ethyl carbamate, 1-methyl-1-phenylethyl carbamate, 1- methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), (3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4- tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1- substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2- picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups are well known in the art and include those described in detail in Protecting Groups in Organic Synthesis, T. W. Greene and P. G. M. Wuts, 3rd edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-y1 (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2- (phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3- methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9- fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a thiol protecting group). Sulfur protecting groups are well known in the art and include those described in detail in Protecting Groups in Organic Synthesis, T. W. Greene and P. G. M. Wuts, 3rd edition, John Wiley & Sons, 1999, incorporated herein by reference.

Exemplary sulfur protecting groups include, but are not limited to, alkyl, benzyl, p-methoxybenzyl, 2,4,6-trimethylbenzyl, 2,4,6-trimethoxybenzyl, o-hydroxybenzyl, p-hydroxybenzyl, o-acetoxybenzyl, p-acetoxybenzyl, p-nitrobenzyl, 4-picolyl, 2-quinolinylmethyl, 2-picolyl N-oxido, 9-anthrylmethyl, 9-fluorenylmethyl, xanthenyl, ferrocenylmethyl, diphenylmethyl, bis(4-methoxyphenyl)methyl, 5-dibenzosuberyl, triphenylmethyl, diphenyl-4-pyridylmethyl, phenyl, 2,4-dinitrophenyl, t-butyl, 1-adamantyl, methoxymethyl (MOM), isobutoxymethyl, benzyloxymethyl, 2-tetrahydropyranyl, benzylthiomethyl, phenylthiomethyl, thiazolidino, acetamidomethyl, trimethylacetamidomethyl, benzamidomethyl, allyloxycarbonylaminomethyl, phenylacetamidomethyl, phthalimidomethyl, acetylmethyl, carboxymethyl, cyanomethyl, (2-nitro-1-phenyl)ethyl, 2-(2,4-dinitrophenyl)ethyl, 2-cyanoethyl, 2-(Trimethylsilyl)ethyl, 2,2-bis(carboethoxy)ethyl, (1-m-nitrophenyl-2-benzoyl)othyl, 2-phenylsulfonylethyl, 2-(4-methylphenylsulfonyl)-2-methylprop-2-yl, acetyl, benzoyl, trifluoroacetyl, N-[[(p-biphenylyl)isopropoxy]carbonyl]-N-methyl]- y-aminothiobutyrate, 2,2,2-trichloroethoxycarbonyl, t-butoxycarbonyl, benzyloxycarbonyl, p-methoxybenzyloxycarbonyl, N-ethyl, N-methoxymethyl, sulfonate, sulfenylthiocarbonate, 3-nitro-2-pyridinesulfenyl sulfide, oxathiolone.

In an embodiment, optionally substituted heteroalkyl is unsubstituted or is substituted with one or more substituents independently selected from —CONH₂, —CO₂H, —COCH₂NH₂, —NH₂, —NH(C═NH)NH₂, or ═O (i.e., two geminal hydrogens on a carbon atom of the heteroalkyl are replaced with the group ═O). In an embodiment, optionally substituted heteroalkyl is unsubstituted. In an embodiment, optionally substituted heteroalkyl is substituted with one or more substituents independently selected from —CONH₂, —CO₂H, —COCH₂NH₂, —NH₂, —NH(C═NH)NH₂, or ═O (i.e., two geminal hydrogens on a carbon atom of the heteroalkyl are replaced with the group ═O).

### III. COMPOUNDS

The present invention relates to novel cyclic peptide inhibitors of the interleukin-23 receptor (IL-23R) or pharmaceutically acceptable salts, solvates, or forms thereof.

In particular, the present invention relates to a cyclic peptide inhibitors of the interleukin-23 receptor (IL-23R) or a pharmaceutically acceptable salt thereof, including those for which a structure is as identified in Table 1A, Table 1B, Table 1C, Table 1D, or Table 1 F of the present specification.

In one aspect, a cyclic peptide inhibitor compound of the interleukin-23 receptor (IL-23R) compound, or a pharmaceutically acceptable salt thereof, has a structure of a compound in Table 1A.

In another aspect, a cyclic peptide inhibitor compound of the interleukin-23 receptor (IL-23R) compound or a pharmaceutically acceptable salt thereof, has a structure of a compound in Table 1B.

In another aspect, a cyclic peptide inhibitor compound of the interleukin-23 receptor (IL-23R) compound or a pharmaceutically acceptable salt thereof, has a structure of a compound in Table 1C.

In another aspect, a cyclic peptide inhibitor compound of the interleukin-23 receptor (IL-23R) compound or a pharmaceutically acceptable salt thereof, has a structure of a compound in Table ID.

In another aspect, a cyclic peptide inhibitor compound of the interleukin-23 receptor (IL-23R) compound or a pharmaceutically acceptable salt thereof, has a structure of a compound in Table 1E.

In another aspect, a cyclic peptide inhibitor compound of the interleukin-23 receptor (IL-23R) compound or a pharmaceutically acceptable salt thereof, has a structure of a compound in Table IF.

Table 1A. Compounds

Lengthy table referenced here
US20260022143A1-20260122-T00001
Please refer to the end of the specification for access instructions.

TABLE 1B
Compounds

| SEQ ID | Harmonized Sequences txt |
|---|---|
| 52 | CF3CO-Pen(3)-N(N(Me)2)-T-7MeW-K(Ac)-Pen(3)-AEF-2Nal-THP-K(Ac)-N- |
| | 3Pya-Sar-CONH2 |
| 66 | CF3CO-Pen(3)-N(N(Me)2)-T-7MeW-K(NMeAc)-Pen(3)-AEF(N(Me)2)-2Nal- |
| | THP-K(NMeAc)-N-3Pya-Sar-CON(Me)2 |
| 68 | CF3CO-Pen(3)-N(N(Me)2)-T-7MeW-K(NMeAc)-Pen(3)-AEF-2Nal-THP- |
| | K(NMeAc)-N-3Pya-Sar-CON(Me)2 |
| 79 | CF3CO-Pen(3)-N(N(Me)2)-T-7MeW-K(NMeAc)-Pen(3)-AEF(N(Me)2)-2Nal- |
| | THP-Q(N(Me)2)-N-3Pya-Sar-CONH2 |
| 91 | 5cpaCO-Pen(3)-N(N(Me)2)-T-7MeW-Q(N(Me)2)-Pen(3)-AEF(N(Me)2)-2Nal- |
| | THP-Q(N(Me)2)-N-3Pya-Sar-CON(Me)2 |
| 93 | CF3CO-K(Me)3-Pen(3)-N(N(Me)2)-T-7MeW-K(NMeAc)-Pen(3)- |
| | AEF(N(Me)2)-2Nal-THP-E-N-3Pya-Sar-CON(Me)2 |
| 94 | CF3CO-Pen(3)-N(N(Me)2)-T-7CF3W-K(NMeAc)-Pen(3)-AEF-2Nal-THP- |
| | K(NMeAc)-N-3Pya-Sar-CON(Me)2 |
| 95 | CF3CO-Pen(3)-N(N(Me)2)-T-7CF3W-K(NMeAc)-Pen(3)-AEF(N(Me)2)-2Nal- |
| | THP-K(NMeAc)-N-3Pya-Sar-CON(Me)2 |
| 96 | CF3CO-k(5cpa)-Pen(3)-N(N(Me)2)-T-7MeW-Q(N(Me)2)-Pen(3)- |
| | AEF(N(Me)2)-2Nal-THP-Q(N(Me)2)-N-3Pya-Sar-CON(Me)2 |
| 97 | 5cpaCO-Pen(3)-N(N(Me)2)-T-7MeW-Q(N(Me)2)-Pen(3)-AEF(N(Me)2)-2Nal- |
| | THP-E-N-3Pya-Sar-CON(Me)2 |
| 98 | MeCO-Pen(3)-N(N(Me)2)-T-7MeW-K(NMeAc)-Pen(3)-AEF(N(Me)2)-2Nal- |
| | THP-Dap(Ac)-N-3Pya-Sar-CON(Me)2 |
| 100 | 5cpaCO-Pen(3)-N(N(Me)2)-T-7MeW-Q(N(Me)2)-Pen(3)-AEF(N(Me)2)-2Nal- |
| | Pip(NMe2)-Q(N(Me)2)-N-3Pya-Sar-CON(Me)2 |
| 101 | 5cpaCO-Pen(3)-N(N(Me)2)-T-7MeW-Q(N(Me)2)-Pen(3)-TMAPF-2Nal-THP- |
| | Q(N(Me)2)-N-3Pya-Sar-CON(Me)2 |
| 104 | CF3CO-Pen(3)-N(N(Me)2)-T-7MeW-K(NMeAc)-Pen(3)-AEF(N(Me)2)-2Nal- |
| | THP-E-N-3Pya-Sar-CON(Me)2 |
| 105 | MeCO-k(Me)3-Pen(3)-N(N(Me)2)-T-7MeW-K(NMeAc)-Pen(3)-AEF(N(Me)2)- |
| | 2Nal-THP-E-N-3Pya-Sar-CON(Me)2 |
| 106 | MeCO-Pen(3)-N(N(Me)2)-T-7MeW-K(NMeAc)-Pen(3)-AEF(N(Me)2)-2Nal- |
| | THP-E-N-3Pya-Sar-CON(Me)2 |
| 117 | MeCO-Pen(3)-N(N(Me)2)-T-7MeW-K(Me)3-Pen(3)-AEF(N(Me)2)-2Nal-THP- |
| | K(Me)3-N-3Pya-Sar-CON(Me)2 |
| 118 | MeCO-Pen(3)-N(N(Me)2)-T-7MeW-Q(N(Me)2)-Pen(3)-AEF(N(Me)2)-2Nal- |
| | THP-Q(N(Me)2)-N-3Pya-Sar-CON(Me)2 |
| 120 | MeCO-Pen(3)-N(N(Me)2)-T-7MeW-Q(N(Me)2)-Pen(3)-AEF(N(Me)2)-2Nal- |
| | THP-E-N-3Pya-Sar-CON(Me)2 |
| 122 | MeCO-Pen(3)-N(N(Me)2)-T-7MeW-K(NMeAc)-Pen(3)-AEF(N(Me)2)-2Nal- |
| | THP-K(NMeAc)-N-3Pya-Sar-CON(Me)2 |
| 129 | MeCO-Pen(3)-N(N(Me)2)-t-7MeW-Dab(NMeAc)-Pen(3)-AEF-2Nal-THP-E-N- |
| | 3Pya-Sar-CON(Me)2 |
| 130 | MeCO-Pen(3)-N(N(Me)2)-T-7MeW-Dab(NMecPEG2a)-Pen(3)-AEF-2Nal- |
| | THP-E-N-3Pya-Sar-CON(Me)2 |
| 131 | MeCO-Pen(3)-N(N(Me)2)-t-7MeW-Dab(NMecarn)-Pen(3)-AEF-2Nal-THP-E- |
| | N-3Pya-Sar-CON(Me)2 |
| 133 | 5cpaCO-Pen(3)-N(N(Me)2)-T-7MeW-K(NMeAc)-Pen(3)-AEF(N(Me)2)-2Nal- |
| | THP-Dap(Ac)-N-3Pya-Sar-CON(Me)2 |
| 139 | cPEG3aCO-Pen(3)-N(N(Me)2)-T-7MeW-Q(N(Me)2)-Pen(3)-AEF-2Nal-THP- |
| | Q(N(Me)2)-N-3Pya-Sar-CON(Me)2 |
| 140 | cPEG5aCO-Pen(3)-N(N(Me)2)-t-7MeW-Dab(NMeAc)-Pen(3)-AEF-2Nal-THP- |
| | Dab(NMeAc)-N-3Pya-Sar-CON(Me)2 |
| 141 | MeCO-Pen(3)-N(N(Me)2)-T-7MeW-Dab(NMeCOmPEG6)-Pen(3)-AEF-2Nal- |
| | THP-Dab(NMeCOmPEG6)-N-3Pya-Sar-CON(Me)2 |
| 142 | cPEG3aCO-Pen(3)-N(N(Me)2)-T-7MeW-AIB-Pen(3)-AEF-2Nal-THP-AIB-N- |
| | 3Pya-Sar-CON(Me)2 |
| 143 | mPEG6CO-SP6-Pen(3)-N(N(Me)2)-T-7MeW-Dab(NMecPEG5a)-Pen(3)-AEF- |
| | 2Nal-THP-E-N-3Pya-Sar-CON(Me)2 |
| 145 | cPEG5aCO-Pen(3)-N(N(Me)2)-T-7MeW-Dab(NMecPEG5a)-Pen(3)-AEF-2Nal- |
| | THP-E-N-3Pya-Sar-CON(Me)2 |
| 146 | mPEG3CO-SP6-Pen(3)-N(N(Me)2)-T-7MeW-Q(N(Me)2)-Pen(3)-AEF-2Nal- |
| | THP-Q(N(Me)2)-N-3Pya-Sar-CONH2 |
| 147 | cPEG3aCO-Pen(3)-N(N(Me)2)-T-7MeW-Q(N(Me)2)-Pen(3)-AEF-2Nal-THP- |
| | Q(N(Me)2)-N-3Pya-Sar-CONH2 |
| 148 | mPEG3CO-SP6-Pen(3)-N(N(Me)2)-T-7MeW-K(NMeAc)-Pen(3)-AEF-2Nal- |
| | THP-K(Ac)-N-3Pya-Sar-CONH2 |
| 149 | cPEG3aCO-Pen(3)-N(N(Me)2)-T-7MeW-K(NMeAc)-Pen(3)-AEF-2Nal-THP- |
| | K(Ac)-N-3Pya-Sar-CONH2 |
| 150 | cPEG3aCO-Pen(3)-N(N(Me)2)-T-7MeW-K(NMeAc)-Pen(3)-AEF-2Nal-THP- |
| | Q(N(Me)2)-N-3Pya-Sar-CONH2 |
| 151 | MeCO-Pen(3)-N(N(Me)2)-t-7MeW-Dab(NMeCarn)-Pen(3)-AEF-2Nal-THP-E- |
| | N-3Pya-Sar-CON(Me)2 |
| 152 | mPEG3CO-SP6-Pen(3)-N(N(Me)2)-T-7MeW-K(NMeAc)-Pen(3)-AEF-2Nal- |
| | THP-QNMe2-N-3Pya-Sar-CONH2 |
| 154 | cPEG3aCO-Pen(3)-N(N(Me)2)-T-7MeW-Q(N(Me)2)-Pen(3)-AEF(N(Me)2)- |
| | 2Nal-THP-Q(N(Me)2)-N-3Pya-Sar-CON(Me)2 |
| 157 | MeCO-Pen(3)-N(N(Me)2)-t-7MeW-Dab(NMeAc)-Pen(3)-AEF-2Nal-THP-AIB- |
| | N-3Pya-Sar-CON(Me)2 |
| 158 | MeCO-Pen(3)-N(N(Me)2)-T-7MeW-Dab(NMeCOmPEG6)-Pen(3)-AEF-2Nal- |
| | THP-E-N-3Pya-Sar-CON(Me)2 |
| 159 | cPEG3aCO-Pen(3)-N(N(Me)2)-T-7MeW-Q(N(Me)2)-Pen(3)-AEF-2Nal-THP- |
| | Dab(NMeAc)-N-3Pya-Sar-CON(Me)2 |
| 160 | cPEG3aCO-Pen(3)-N(N(Me)2)-t-7MeW-Dab(NMeAc)-Pen(3)-AEF-2Nal-THP- |
| | Dab(NMeAc)-N-3Pya-Sar-CON(Me)2 |
| 163 | MeCO-hk(Me)3-Pen(3)-N(N(Me)2)-T-7MeW-K(NMeAc)-Pen(3)-AEF-2Nal- |
| | THP-E-N-3Pya-Sar-CON(Me)2 |
| 164 | cPEG3aCO-Pen(3)-N(N(Me)2)-T-7MeW-K(NMeAc)-Pen(3)-TMAPF-2Nal- |
| | THP-K(NMeAc)-N-3Pya-Sar-CON(Me)2 |
| 165 | cPEG3aCO-Pen(3)-N(N(Me)2)-T-7MeW-K(NMeAc)-Pen(3)-TMAPF-2Nal- |
| | THP-E-N-3Pya-Sar-CON(Me)2 |
| 169 | cPEG3aCO-Pen(3)-N(N(Me)2)-T-7MeW-K(NMeAc)-Pen(3)-AEF-2Nal-THP- |
| | K(NMeAc)-N-3Pya-Sar-CONH2 |
| 170 | MeCO-Pen(3)-N(N(Me)2)-t-7MeW-Dab(NMeAc)-Pen(3)-AEF-2Nal-THP- |
| | Dab(NMeAc)-N-3Pya-Sar-CON(Me)2 |
| 171 | cPEG3aCO-Pen(3)-N(N(Me)2)-t-7MeW-Dab(NMeAc)-Pen(3)-AEF-2Nal-THP- |
| | K(NMeAc)-N-3Pya-Sar-CONH2 |
| 172 | cPEG5aCO-Pen(3)-N(N(Me)2)-T-7MeW-Dab(NMecPEG2a)-Pen(3)-AEF-2Nal- |
| | THP-Dab(NMecPEG2a)-N-3Pya-Sar-CON(Me)2 |
| 173 | cPEG5aCO-Pen(3)-N(N(Me)2)-T-7MeW-Q(N(Me)2)-Pen(3)-AEF-2Nal-THP- |
| | Dab(NMeAc)-N-3Pya-Sar-CON(Me)2 |
| 175 | MeCO-Pen(3)-N(N(Me)2)-T-7MeW-Dab(NMecPEG3a)-Pen(3)-AEF-2Nal- |
| | THP-Dab(NMecPEG3a)-N-3Pya-Sar-CON(Me)2 |
| 176 | cPEG3aCO-Pen(3)-N(N(Me)2)-t-7MeW-Dab(NMeAc)-Pen(3)-AEF-2Nal-THP- |
| | E-N-3Pya-Sar-CON(Me)2 |
| 177 | cPEG3aCO-Pen(3)-N(N(Me)2)-T-7MeW-K(NMeAc)-Pen(3)-AEF-2Nal-THP- |
| | Dab(NMeAc)-N-3Pya-Sar-CON(Me)2 |
| 179 | cPEG3aCO-Pen(3)-N(N(Me)2)-T-7MeW-K(NMeAc)-Pen(3)-AEF-2Nal-diFCpx- |
| | Dab(NMeAc)-N-3Pya-Sar-CON(Me)2 |
| 180 | cPEG3aCO-Pen(3)-N(N(Me)2)-T-7MeW-K(NMeAc)-Pen(3)-AEF-2Nal-diFCpx- |
| | Dab(NMeAc)-N-3Pya-Sar-CON(Me)2 |
| 181 | cPEG3aCO-Pen(3)-N(N(Me)2)-T-7MeW-K(NMeAc)-Pen(3)-APEG3F-2Nal- |
| | THP-K(NMeAc)-N-3Pya-Sar-CON(Me)2 |
| 182 | MeCO-Pen(3)-N(N(Me)2)-T-7MeW-K(NMeAc)-Pen(3)-AEF-2Nal-THP- |
| | K(NMeAc)-N-3Pya-Sar-CONH2 |
| 183 | MeCO-Pen(3)-N(N(Me)2)-T-7MeW-K(NMeAc)-Pen(3)-APEG3F-2Nal-THP- |
| | K(NMeAc)-N-3Pya-Sar-CON(Me)2 |
| 184 | cPEG3aCO-Pen(3)-N(N(Me)2)-T-7MeW-K(NMeAc)-Pen(3)-APEG3F-2Nal- |
| | THP-E-N-3Pya-Sar-CON(Me)2 |
| 187 | cPEG3aCO-Pen(3)-N(N(Me)2)-T-7MeW-K(NMeAc)-Pen(3)-4DMPEF-2Nal- |
| | THP-K(NMeAc)-N-3Pya-Sar-CON(Me)2 |
| 190 | MeCO-Pen(3)-N(N(Me)2)-T-7MeW-K(NMeAc)-Pen(3)-TMAPF-2Nal-THP- |
| | K(NMeAc)-N-3Pya-Sar-CON(Me)2 |
| 191 | cPEG3aCO-Pen(3)-N(N(Me)2)-t-7MeW-Dab(NMeAc)-Pen(3)-AEF-2Nal-THP- |
| | Dab(NMeAc)-N-3Pya-Sar-CONH(PEG3a) |
| 193 | cPEG3aCO-Pen(3)-N(N(Me)2)-T-7MeW-K(NMeAc)-Pen(3)-4DMPzEF-2Nal- |
| | THP-K(NMeAc)-N-3Pya-Sar-CON(Me)2 |
| 196 | cPEG3aCO-Pen(3)-N(N(Me)2)-T-7MeW-Dab(NMecPEG3a)-Pen(3)-AEF-2Nal- |
| | THP-Dab(NMeAc)-N-3Pya-Sar-CON(Me)2 |
| 197 | mPEG3CO-SP6-Pen(3)-N(N(Me)2)-T-7MeW-K(NMeAc)-Pen(3)-APEG3F- |
| | 2Nal-THP-Dab(NMeAc)-N-3Pya-Sar-CON(Me)2 |
| 198 | cPEG3aCO-Pen(3)-N(N(Me)2)-T-7MeW-K(NMeac)-Pen(3)-MMoEF-2Nal- |
| | THP-K(NMeAc)-N-3Pya-Sar-CON(Me)2 |
| 199 | cPEG3aCO-Pen(3)-N(N(Me)2)-T-7MeW-K(NMeAc)-Pen(3)-APEG2F-2Nal- |
| | THP-K(NMeAc)-N-3Pya-Sar-CON(Me)2 |
| 202 | cPEG3aCO-Pen(3)-N(N(Me)2)-T-7MeW-K(NMeAc)-Pen(3)-DMPMF-2Nal- |
| | THP-K(NMeAc)-N-3Pya-Sar-CON(Me)2 |
| 203 | cPEG3aCO-Pen(3)-N(N(Me)2)-T-7MeW-K(NMeAc)-Pen(3)-AEF(BisMEP))- |
| | 2Nal-THP-K(NMeAc)-N-3Pya-Sar-CON(Me)2 |
| 206 | cPEG3aCO-Pen(3)-N(N(Me)2)-T-7MeW-K(NMeAc)-Pen(3)-AEF(NsCh)-2Nal- |
| | THP-K(NMeAc)-N-3Pya-Sar-CON(Me)2 |
| 207 | cPEG3aCO-Pen(3)-N(N(Me)2)-T-7MeW-K(NMeAc)-Pen(3)-AEF(AcCh)-2Nal- |
| | THP-K(NMeAc)-N-3Pya-Sar-CON(Me)2 |
| 208 | cPEG3aCO-Pen(3)-N(N(Me)2)-T-7MeW-K(NMeAc)-Pen(3)-AEF((Ch)cPEG3a)- |
| | 2Nal-THP-K(NMeAc)-N-3Pya-Sar-CON(Me)2 |
| 209 | cPEG3aCO-Pen(3)-N(N(Me)2)-T-7MeW-K(NMeAc)-Pen(3)-APEG3F-2Nal- |
| | THP-Dab(NMeAc)-N-3Pya-Sar-CON(Me)2 |
| 210 | cPEG3aCO-Pen(3)-N(N(Me)2)-T-7MeW-K(NMeAc)-Pen(3)-ACHMF-2Nal- |
| | THP-K(NMeAc)-N-3Pya-Sar-CON(Me)2 |
| 211 | cPEG3aCo-Pen(3)-N(NMe2)-T-7MeW-K(NMeAc)-Pen(3)-AEF(NHCh)-2Nal- |
| | THP-K(NMeAc)-N-3Pya-Sar-CON(Me)2 |
| 212 | cPEG3aCO-Pen(3)-N(N(Me)2)-T-7MeW-K(NMeAc)-Pen(3)-APEG3F-2Nal- |
| | THP-Dab(NMeAc)-N-3Pya-Sar-CON(Me)2 |
| 213 | MeCO-k(d)-Pen(3)-N(N(Me)2)-T-7MeW-K(NMeAc)-Pen(3)-APEG3F-2Nal- |
| | THP-Dab(NMeAc)-N-3Pya-Sar-CON(Me)2 |
| 214 | MeCO-Pen(3)-N(N(Me)2)-t-7MeW-Dab(NMecarn)-Pen(3)-APEG3F-2Nal- |
| | THP-K(NMeAc)-N-3Pya-Sar-CON(Me)2 |
| 215 | cPEG3aCO-Pen(3)-N(N(Me)2)-T-7MeW-K(NMeAc)-Pen(3)-DMMMF-2Nal- |
| | THP-K(NMeAc)-N-3Pya-Sar-CON(Me)2 |
| 216 | MeCO-k(Me)3-Pen(3)-N(N(Me)2)-T-7MeW-K(NMeAc)-Pen(3)-APEG3F-2Nal- |
| | THP-K(NMeAc)-N-3Pya-Sar-CON(Me)2 |
| 217 | MeCO-Pen(3)-N(N(Me)2)-T-7MeW-K(NMePEG3a)-Pen(3)-APEG3F-2Nal- |
| | THP-K(NMeAc)-N-3Pya-Sar-CON(Me)2 |
| 218 | MeCO-Pen(3)-N(N(Me)2)-T-7MeW-K(NMeAc)-Pen(3)-APEG3F-2Nal-THP- |
| | K(NMePEG3a)-N-3Pya-Sar-CON(Me)2 |
| 219 | MeCO-Pen(3)-N(N(Me)2)-T-7MeW-K(NMeAc)-Pen(3)-TMAPF-2Nal-THP- |
| | K(NMeAc)-N-3Pya-Sar-CO(NHPEG3a) |
| 220 | cPEG3aCO-Pen(3)-N(N(Me)2)-T-7MeW-K(NMeAc)-Pen(3)-TMAPF-2Nal- |
| | THP-K(NMeAc)-N-3Pya-Sar-CO(NHPEG3a) |
| 221 | cPEG3aCO-Pen(3)-N(N(Me)2)-T-7MeW-K(NMeAc)-Pen(3)-AEF(BisMEPa)- |
| | 2Nal-THP-K(NMeAc)-N-3Pya-Sar-CON(Me)2Â |
| 222 | cPEG3aCO-Pen(3)-N(N(Me)2)-T-7MeW-K(NMeAc)-Pen(3)-ACHMF-2Nal- |
| | THP-K(NMeAc)-N-3Pya-Sar-CON(Me)2 |
| 223 | cPEG3aCO-Pen(3)-N(N(Me)2)-T-7MeW-K(NMeAc)-Pen(3)-MMPEG3F-2Nal- |
| | THP-K(NMeAc)-N-3Pya-Sar-CON(Me)2 |
| 224 | cPEG3aCO-Pen(3)-N(N(Me)2)-T-7MeW-K(NMeAc)-Pen(3)-MPzPEG3F-2Nal- |
| | THP-K(NMeAc)-N-3Pya-Sar-CON(Me)2 |
| 225 | mPEG3CO-SP6-Pen(3)-N(N(Me)2)-T-7MeW-K(NMeAc)-Pen(3)-APEG3F- |
| | 2Nal-THP-K(NMeAc)-N-3Pya-Sar-CON(Me)2 |
| 226 | cPEG3aCO-Pen(3)-N(N(Me)2)-T-7MeW-K(NMeAc)-Pen(3)-TMA4F-2Nal- |
| | THP-K(NMeAc)-N-3Pya-Sar-CON(Me)2 |
| 227 | cPEG3aCO-Pen(3)-N(N(Me)2)-T-7MeW-K(NMeAc)-Pen(3)-mPEG2TMA4F- |
| | 2Nal-THP-K(NMeAc)-N-3Pya-Sar-CON(Me)2 |
| 228 | cPEG3aCO-Pen(3)-N(N(Me)2)-T-7MeW-K(NMeAc)-Pen(3)-mPEG2TMA2F- |
| | 2Nal-THP-K(NMeAc)-N-3Pya-Sar-CON(Me)2 |
| 229 | cPEG3aCO-Pen(3)-N(N(Me)2)-T-7MeW-K(NMeAc)-Pen(3)-TMA3F-2Nal- |
| | THP-K(NMeAc)-N-3Pya-Sar-CON(Me)2 |
| 230 | cPEG3aCO-Pen(3)-N(N(Me)2)-T-7MeW-K(NMeAc)-Pen(3)-TMA3F-2Nal- |
| | THP-K(NMeAc)-N-3Pya-Sar-CON(Me)2 |
| 231 | cPEG3aCO-Pen(3)-N(N(Me)2)-T-7MeW-K(NMeac)-Pen(3)-NPyEF-2Nal-THP- |
| | K(NMeAc)-N-3Pya-Sar-CON(Me)2 |
| 232 | MeCO-APEG2ser-Pen(3)-N(N(Me)2)-T-7MeW-K(NMeAc)-Pen(3)-APEG3F- |
| | 2Nal-THP-K(NMeAc)-N-3Pya-Sar-CON(Me)2 |
| 233 | cPEG3aCO-Pen(3)-N(N(Me)2)-T-7MeW-K(NMeAc)-Pen(3)-APEG3F-2Nal- |
| | THP-K(Ac)-N-3Pya-Sar-CON(Me)2 |
| 234 | cPEG3aCO-Pen(3)-N(N(Me)2)-T-7MeW-K(NMeAc)-Pen(3)-AEF(MePrpa)- |
| | 2Nal-THP-K(NMeAc)-N-3Pya-Sar-CON(Me)2 |
| 236 | cPEG3aCO-Pen(3)-N(N(Me)2)-T-7MeW-K(NMeAc)-Pen(3)-Y(C9OH)-2Nal- |
| | THP-K(NMeAc)-N-3Pya-Sar-CON(Me)2 |
| 237 | cPEG3aCO-Pen(3)-N(N(Me)2)-T-7MeW-K(NMeAc)-Pen(3)-AEF(NMePEG2a)- |
| | 2Nal-THP-K(NMeAc)-N-3Pya-Sar-CON(Me)2 |
| 238 | cPEG3aCO-Pen(3)-N(N(Me)2)-T-7MeW-K(NMeAc)-Pen(3)-AEF(PEG2a)- |
| | 2Nal-THP-K(NMeAc)-N-3Pya-Sar-CON(Me)2 |
| 239 | cPEG3aCO-Pen(3)-N(N(Me)2)-T-7MeW-K(NMeAc)-pen(3)- |
| | AEF(NMe2mPEG3)-2Nal-THP-K(NMeAc)-N-3Pya-Sar-CON(Me)2 |
| 241 | cPEG3aCO-Pen(3)-N(N(Me)2)-T-7MeW-K(NMeAc)-Pen(3)- |
| | AEF(NMeBismPEG3)-2Nal-THP-K(NMeAc)-N-3Pya-Sar-CON(Me)2 |
| 242 | cPEG3aCO-Pen(3)-N(N(Me)2)-T-7MeW-K(NMeAc)-Pen(3)-YC8COPip-2Nal- |
| | THP-K(NMeAc)-N-3Pya-Sar-CON(Me)2 |
| 243 | cPEG3aCO-Pen(3)-N(N(Me)2)-T-7MeW-K(Ac)-Pen(3)-APEG3F-2Nal-THP- |
| | K(NMeAc)-N-3Pya-Sar-CON(Me)2 |
| 244 | MeCO-Pen(3)-N(N(Me)2)-T-7MeW-K(NMeAc)-Pen(3)-APEG3F-2Nal-THP- |
| | APEG2Ser(S*)-N-3Pya-Sar-CON(Me)2 |
| 245 | cPEG3aCO-Pen(3)-N(N(Me)2)-T-7MeW-APEG2Ser-Pen(3)-AEF-2Nal-THP- |
| | K(NMeAc)-N-3Pya-Sar-CON(Me)2 |
| 246 | cPEG3aCO-Pen(3)-N(N(Me)2)-T-7MeW-APEG2Ser(R*)-Pen(3)-AEF-2Nal- |
| | THP-K(NMeAc)-N-3Pya-Sar-CON(Me)2 |
| 247 | cPEG3aCO-Pen(3)-N(N(Me)2)-T-7MeW-APEG2Ser(S*)-Pen(3)-TMAPF-2Nal- |
| | THP-K(NMeAc)-N-3Pya-Sar-CON(Me)2 |
| 248 | cPEG3aCO-Pen(3)-N(N(Me)2)-T-7MeW-APEG2Ser(R*)-Pen(3)-TMAPF-2Nal- |
| | THP-K(NMeAc)-N-3Pya-Sar-CON(Me)2 |
| 254 | cPEG3aCO-Pen(3)-N(N(Me)2)-T-7MeW-K(NMeAc)-Pen(3)- |
| | YC8CO(NHPEG3a)-2Nal-THP-K(NMeAc)-N-3Pya-Sar-CON(Me)2 |
| 255 | MeCO-Pen(3)-N(N(Me)2)-T-7MeW-APEG2Ser(R*)-Pen(3)-APEG3F-2Nal- |
| | THP-APEG2Ser(S*)-N-3Pya-Sar-CON(Me)2 |
| 256 | MeCO-Pen(3)-N(N(Me)2)-T-7MeW-APEG2Ser(S*)-Pen(3)-APEG3F-2Nal- |
| | THP-APEG2Ser(S*)-N-3Pya-Sar-CON(Me)2 |
| 257 | MeCO-Pen(3)-N(N(Me)2)-t-7MeW-Dab(NMecarn)-Pen(3)-APEG3F-2Nal- |
| | THP-Dab(NMeAc)-N-3Pya-Sar-CON(Me)2 |
| 261 | cPEG3aCO-Pen(3)-N(N(Me)2)-T-7MeW-K(NMeAc)-pen(3)-AEF(NMe3)-2Nal- |
| | THP-K(NMeAc)-N-3Pya-Sar-CON(Me)2 |
| 262 | cPEG3aCO-Pen(3)-N(N(Me)2)-T-7MeW-K(NMeAc)-Pen(3)-MTASF-2Nal- |
| | THP-K(NMeAc)-N-3Pya-Sar-CON(Me)2 |
| 263 | cPEG3aCO-Pen(3)-N(N(Me)2)-T-7MeW-K(NMeAc)-Pen(3)-TBAPEG3F-2Nal- |
| | THP-K(NMeAc)-N-3Pya-Sar-CON(Me)2 |
| 264 | cPEG3aCO-Pen(3)-N(N(Me)2)-T-7MeW-K(NMeAc)-Pen(3)-AEF(aPEG2a)- |
| | 2Nal-THP-K(NMeAc)-N-3Pya-Sar-CON(Me)2 |
| 265 | cPEG3aCO-Pen(3)-N(N(Me)2)-T-7MeW-K(NMeAc)-Pen(3)- |
| | AEF(BisPEG2a)(RS)-2Nal-THP-K(NMeAc)-N-3Pya-Sar-CON(Me)2 |
| 266 | cPEG3aCO-Pen(3)-N(N(Me)2)-T-7MeW-K(NMeAc)-Pen(3)- |
| | AEF(BisPEG2a)(S*)-2Nal-THP-K(NMeAc)-N-3Pya-Sar-CON(Me)2 |
| 267 | MeCO-Pen(3)-N(N(Me)2)-T-7MeW-APEG2Ser(S*)-Pen(3)-APEG3F-2Nal- |
| | THP-K(NMeAc)-N-3Pya-Sar-CON(Me)2 |
| 268 | MeCO-Pen(3)-N(N(Me)2)-T-7MeW-APEG2Ser(R*)-Pen(3)-APEG3F-2Nal- |
| | THP-K(NMeAc)-N-3Pya-Sar-CON(Me)2 |
| 269 | cPEG3aCO-Pen(3)-N(N(Me)2)-T-7MeW-K(NMeAc)-Pen(3)-DMTASF-2Nal- |
| | THP-K(NMeAc)-N-3Pya-Sar-CON(Me)2 |
| 271 | cPEG3aCO-Pen(3)-N(N(Me)2)-T-7MeW-K(NMeAc)-Pen(3)-morfTMA4F- |
| | 2Nal-THP-K(NMeAc)-N-3Pya-Sar-CON(Me)2 |
| 272 | cPEG3aCO-Pen(3)-N(N(Me)2)-T-7MeW-K(NMeAc)-Pen(3)-mPEG3TMA4F- |
| | 2Nal-THP-K(NMeAc)-N-3Pya-Sar-CON(Me)2 |
| 273 | cPEG3aCO-Pen(3)-N(N(Me)2)-T-7MeW-K(NMeAc)-Pen(3)-PiperazinequatF- |
| | 2Nal-THP-K(NMeAc)-N-3Pya-Sar-CON(Me)2 |
| 274 | cPEG3aCO-Pen(3)-N(N(Me)2)-T-7MeW-K(NMeac)-Pen(3)-MMoPF-2Nal- |
| | THP-K(NMeAc)-N-3Pya-Sar-CON(Me)2 |
| 275 | MeCO-Pen(3)-N(N(Me)2)-T-7MeW-K(NMeAc)-Pen(3)-TMAPF-2Nal-THP- |
| | APEG2Ser-N-3Pya-Sar-CON(Me)2 |
| 277 | cPEG3aCO-Pen(3)-N(N(Me)2)-T-7MeW-K(NMeAc)-Pen(3)-AEF(SPD)-2Nal- |
| | THP-K(NMeAc)-N-3Pya-Sar-CON(Me)2 |
| 278 | MeCO-Pen(3)-N(N(Me)2)-t-7MeW-Dab(NMecarn)-Pen(3)-APEG3F-2Nal- |
| | THP-Dab(NMecarn)-N-3Pya-Sar-CON(Me)2 |
| 279 | MeCO-Pen(3)-N(N(Me)2)-T-7MeW-K(Ac)-Pen(3)-AEF-2Nal-THP-K(Ac)-N- |
| | 3Pya-Sar-CONH2 |
| 283 | cPEG3aCO-Pen(3)-N(N(Me)2)-T-7MeW-K(NMeAc)-Pen(3)-Y(OTzlChmPEG3)- |
| | 2Nal-THP-K(NMeAc)-N-3Pya-Sar-CON(Me)2 |
| 284 | cPEG3aCO-Pen(3)-N(N(Me)2)-T-7MeW-K(NMeAc)-Pen(3)-Y(OTzlTMA4)- |
| | 2Nal-THP-K(NMeAc)-N-3Pya-Sar-CON(Me)2 |
| 285 | cPEG3aCO-Pen(3)-N(N(Me)2)-T-7MeW-K(NMeAc)-Pen(3)-Y(OTzlChmPEG)- |
| | 2Nal-THP-K(NMeAc)-N-3Pya-Sar-CON(Me)2 |
| 286 | cPEG3aCO-Pen(3)-N(N(Me)2)-T-7MeW-K(NMeAc)-Pen(3)-Y(OTzlCh)-2Nal- |
| | THP-K(NMeAc)-N-3Pya-Sar-CON(Me)2 |
| 287 | cPEG3aCO-Pen(3)-N(N(Me)2)-T-7MeW-K(NMeAc)-Pen(3)-Tzl(Ch)-2Nal-THP- |
| | K(NMeAc)-N-3Pya-Sar-CON(Me)2 |
| 288 | cPEG3aCO-Pen(3)-N(N(Me)2)-T-7MeW-K(NMeAc)-Pen(3)-TzlChmPEG-2Nal- |
| | THP-K(NMeAc)-N-3Pya-Sar-CON(Me)2 |
| 289 | cPEG3aCO-Pen(3)-N(N(Me)2)-T-7MeW-K(NMeAc)-Pen(3)-TzlChmPEG3- |
| | 2Nal-THP-K(NMeAc)-N-3Pya-Sar-CON(Me)2 |
| 290 | cPEG3aCO-Pen(3)-N(N(Me)2)-T-7MeW-K(NMeAc)-Pen(3)-Y(OtzlC1aC8)- |
| | 2Nal-THP-K(NMeAc)-N-3Pya-Sar-CON(Me)2 |
| 291 | cPEG3aCO-Pen(3)-N(N(Me)2)-T-7MeW-K(NMeAc)-Pen(3)-Y(OTzlChC16)- |
| | 2Nal-THP-K(NMeAc)-N-3Pya-Sar-CON(Me)2 |
| 292 | cPEG3aCO-Pen(3)-N(N(Me)2)-T-7MeW-K(NMeAc)-Pen(3)- |
| | Diazabiclyclooctane6F-2Nal-THP-K(NMeAc)-N-3Pya-Sar-CON(Me)2 |
| 293 | cPEG3aCO-Pen(3)-N(N(Me)2)-T-7MeW-K(NMeAc)-Pen(3)-TMA6F-2Nal- |
| | THP-K(NMeAc)-N-3Pya-Sar-CON(Me)2 |
| 294 | cPEG3aCO-Pen(3)-N(N(me)2)-T-7(3NAcPh)W-K(NMeAc)-Pen(3)-TMAPF- |
| | 2Nal-THP-K(NMeAc)-N-3Pya-Sar-CON(Me)2 |
| 295 | cPEG3aCO-Pen(3)-N(N(Me)2)-T-7MeW-K(NMeAc)-Pen(3)-TMA8F-2Nal- |
| | THP-K(NMeAc)-N-3Pya-Sar-CON(Me)2 |
| 297 | cPEG3aCO-Pen(3)-N(N(Me)2)-T-7MeW-K(NMeAc)-Pen(3)-NPyPEG3F-2Nal- |
| | THP-K(NMeAc)-N-3Pya-Sar-CON(Me)2 |
| 298 | cPEG3aCO-Pen(3)-N(N(Me)2)-T-7MeW-K(NMeAc)-Pen(3)-dFPPEG3F-2Nal- |
| | THP-K(NMeAc)-N-3Pya-Sar-CON(Me)2 |
| 301 | cPEG3aCO-Pen(3)-N(N(Me)2)-T-7MeW-K(NMeAC)-Pen(3)-TMAPF-2Nal- |
| | THP-Dab(NMeAc)-N-3Pya-Sar-CON(Me)2 |
| 302 | cPEG3aCO-Pen(3)-N(N(Me)2)-T-7MeW-K(NMeAc)-Pen(3)-F(4TzlTMA4)- |
| | 2Nal-THP-K(NMeAc)-N-3Pya-Sar-CON(Me)2 |
| 303 | cPEG3aCO-Pen(3)-N(N(Me)2)-T-7MeW-K(NMeAc)-Pen(3)-F(4TzlMME)- |
| | 2Nal-THP-K(NMeAc)-N-3Pya-Sar-CON(Me)2 |
| 304 | cPEG3aCO-Pen(3)-N(N(Me)2)-T-7MeW-K(NMeAc)-Pen(3)-F(4G)-2Nal-THP- |
| | K(NMeAc)-N-3Pya-Sar-CON(Me)2 |
| 305 | cPEG3aCO-Pen(3)-N(N(Me)2)-T-7MeW-K(NMeAc)-Pen(3)-Y(OEOXlMECh)- |
| | 2Nal-THP-K(NMeAc)-N-3Pya-Sar-CON(Me)2 |
| 306 | cPEG3aCO-Pen(3)-N(N(Me)2)-T-7MeW-K(NMeAc)-Pen(3)-Y(OZOXlMECh)- |
| | 2Nal-THP-K(NMeAc)-N-3Pya-Sar-CON(Me)2 |
| 307 | cPEG3aCO-Pen(3)-N(N(Me)2)-T-7MeW-K(NMeAC)-Pen(3)-TMAPF-2Nal- |
| | THP-K(NMeaC)-N-3Pya-NMeK(PEG2PEG2C12)-CONH2 |
| 308 | cPEG3aCO-Pen(3)-N(N(Me)2)-T-7MeW-K(Ac)-Pen(3)-TMAPF-2Nal-THP- |
| | Dab(NMeAc)-N-3Pya-Sar-CON(Me)2 |
| 310 | cPEG3aCO-Pen(3)-N(N(Me)2)-T-7MeW-K(NMeAC)-Pen(3)-TMAPF-2Nal- |
| | THP-Q(N(Me)2)-N-3Pya-Sar-CON(Me)2 |
| 312 | cPEG3aCO-Pen(3)-N(N(Me)2)-T-7MeW-Q(N(Me)2)-Pen(3)-TMAPF-2Nal- |
| | THP-K(NMeAC)-N-3Pya-Sar-CON(Me)2 |
| 313 | cPEG3aCO-Pen(3)-N(N(Me)2)-T-7MeW-K(Ac)-Pen(3)-TMAPF-2Nal-THP- |
| | K(NMeaC)-N-3Pya-NMeK(PEG2PEG2gEC12)-CONH2 |
| 314 | cPEG3aCO-Pen(3)-N(N(Me)2)-T-7MeW-K(NMeAC)-Pen(3)-TMAPF-2Nal- |
| | THP-K(NMeaC)-N-3Pya-NMeK(PEG2PEG2gEC12)-CONH2 |
| 315 | cPEG3aCO-Pen(3)-N(N(Me)2)-T-7MeW-K(NMeAC)-Pen(3)-TMAPF-2Nal- |
| | THP-K(NMeaC)-N-3Pya-NMeK(PEG2PEG2gEC14)-CONH2 |
| 316 | cPEG3aCO-Pen(3)-N(N(Me)2)-T-7MeW-K(NMeAc)-Pen(3)-TMAPF-2Nal- |
| | THP-A-N-3Pya-Sar-CON(Me)2 |
| 318 | MeCO-APEG2Ser(S*)-Pen(3)-N(N(Me)2)-T-7MeW-K(NMeAc)-Pen(3)- |
| | APEG3F-2Nal-THP-K(NMeAc)-N-3Pya-Sar-CON(Me)2 |
| 319 | MeCO-APEG2Ser(R*)-Pen(3)-N(N(Me)2)-T-7MeW-K(NMeAc)-Pen(3)- |
| | APEG3F-2Nal-THP-K(NMeAc)-N-3Pya-Sar-CON(Me)2 |
| 320 | cPEG3aCO-Pen(3)-N(N(Me)2)-T-7MeW-K(NMeAc)-Pen(3)-AEF-2Nal-THP- |
| | K(NMeAc)-N-3Pya-Sar-CON(Me)2 |
| 321 | cPEG3aCO-Pen(3)-N(N(Me)2)-T-7MeW-K(NMeAc)-Pen(3)-AEF(N(Me)2)- |
| | 2Nal-THP-K(NMeAc)-N-3Pya-Sar-CON(Me)2 |
| 322 | C12gEPEG2PEG2CO-hk(Me)3-Pen(3)-N(N(Me)2)-T-7MeW-K(NMeAc)- |
| | Pen(3)-TMAPF-2Nal-THP-K(NMeAc)-N-3Pya-Sar-CON(Me)2 |
| 323 | C12gEPEG2PEG2CO-hk(Me)3-Pen(3)-N(N(Me)2)-T-7MeW- |
| | K(PEG2PEG2gEC12)-Pen(3)-TMAPF-2Nal-THP-K(NMeAc)-N-3Pya-Sar- |
| | CON(Me)2 |
| 324 | C14gEPEG2PEG2CO-hk(Me)3-Pen(3)-N(N(Me)2)-T-7MeW-K(NMeAc)- |
| | Pen(3)-TMAPF-2Nal-THP-K(NMeAc)-N-3Pya-Sar-CON(Me)2 |
| 325 | C14gEPEG2PEG2CO-hk(Me)3-Pen(3)-N(N(Me)2)-T-7MeW- |
| | K(PEG2PEG2gEC14)-Pen(3)-TMAPF-2Nal-THP-K(NMeAc)-N-3Pya-Sar- |
| | CON(Me)2 |
| 326 | cPEG3aCO-Pen(3)-N(N(Me)2)-T-7MeW-K(NMeAc)-Pen(3)-TMAPF-2Nal- |
| | THP-K(NMeAc)-N-3Pya-Sar-CONH2 |
| 327 | cPEG3aCO-Pen(3)-N(N(Me)2)-T-7MeW-K(NMeAc)-Pen(3)-TMAPF-2Nal- |
| | THP-Cit-N-3Pya-Sar-CON(Me)2 |
| 329 | cPEG3aCO-Pen(3)-N(N(Me)2)-T-7MeW-K(Me)3-Pen(3)-TMAPF-2Nal-THP- |
| | K(NMeAc)-N-3Pya-Sar-CON(Me)2 |
| 330 | cPEG3aCO-Pen(3)-N(N(Me)2)-T-7MeW-K(NMeAc)-Pen(3)-TMAPF-2nal- |
| | Acpx-K(NMeAc)-N-3Pya-Sar-CON(Me)2 |
| 331 | cPEG3aCO-Pen(3)-N(N(Me)2)-T-7MeW-K(NMeAc)-Pen(3)-F(4N3)-2Nal-THP- |
| | K(NMeAc)-N-3Pya-Sar-CON(Me)2 |
| 332 | cPEG3aCO-Pen(3)-N(N(Me)2)-T-7MeW-K(NMeAc)-Pen(3)-F(4TzlTMA1)- |
| | 2Nal-THP-K(NMeAc)-N-3Pya-Sar-CON(Me)2 |
| 333 | cPEG3aCO-Pen(3)-N(N(Me)2)-T-7MeW-K(NMeAc)-Pen(3)-F(4TzlTMA2)- |
| | 2Nal-THP-K(NMeAc)-N-3Pya-Sar-CON(Me)2 |
| 334 | cPEG3aCO-Pen(3)-N(N(Me)2)-T-7MeW-K(NMeAc)-Pen(3)-F(4TzlTMA3)- |
| | 2Nal-THP-K(NMeAc)-N-3Pya-Sar-CON(Me)2 |
| 335 | cPEG3aCO-Pen(3)-N(N(Me)2)-T-7MeW-K(NMeAc)-Pen(3)-F(4TzlTMA5)- |
| | 2Nal-THP-K(NMeAc)-N-3Pya-Sar-CON(Me)2 |
| 336 | cPEG3aCO-Pen(3)-N(N(Me)2)-T-7MeW-K(NMeAc)-Pen(3)-F(4TzlMMo4)- |
| | 2Nal-THP-K(NMeAc)-N-3Pya-Sar-CON(Me)2 |
| 337 | cPEG3aCO-Pen(3)-N(N(Me)2)-T-7MeW-K(NMeAc)-Pen(3)- |
| | F(4TzlDMA4mPEG)-2Nal-THP-K(NMeAc)-N-3Pya-Sar-CON(Me)2 |
| 338 | cPEG3aCO-Pen(3)-N(N(Me)2)-T-7MeW-K(NMeAc)-Pen(3)-hFTMAPF-2Nal- |
| | THP-K(NMeAc)-N-3Pya-Sar-CON(Me)2 |
| 339 | cPEG3aCO-Pen(3)-N(N(Me)2)-T-7MeW-K(NMeAc)-Pen(3)-TMAPF-2nal- |
| | Achx-K(NMeAc)-N-3Pya-Sar-CON(Me)2 |
| 340 | cPEG3aCO-Pen(3)-N(N(Me)2)-T-7MeW-K(NMeAc)-Pen(3)-TMAPF-2Nal-Chg- |
| | K(NMeAc)-N-3Pya-Sar-CON(Me)2 |
| 341 | cPEG3aCO-Pen(3)-N(N(Me)2)-T-7MeW-K(NMeAc)-Pen(3)-TMAPF-2Nal-Aib- |
| | K(NMeAc)-N-3Pya-Sar-CON(Me)2 |
| 343 | 5cpaCO-Pen(3)-N(N(Me)2)-T-7MeW-K(NMeAc)-Pen(3)-TMAPF-2Nal-THP- |
| | K(NMeAc)-N-3Pya-Sar-CON(Me)2 |
| 344 | MeCO-hk(Me)3-Pen(3)-N(N(Me)2)-T-7MeW-K(NMeAc)-Pen(3)-TMAPF- |
| | 2Nal-THP-K(NMeAc)-N-3Pya-Sar-CON(Me)2 |
| 345 | MeCO-r-Pen(3)-N(N(Me)2)-T-7MeW-K(NMeAc)-Pen(3)-TMAPF-2Nal-THP- |
| | K(NMeAc)-N-3Pya-Sar-CON(Me)2 |
| 346 | cPEG3aCO-Pen(3)-N(N(Me)2)-T-7MeW-Cit-Pen(3)-TMAPF-2Nal-THP- |
| | K(NMeAc)-N-3Pya-Sar-CON(Me)2 |
| 347 | cPEG3aCO-k(PEG2PEG2gEC14)-Pen(3)-N(N(Me)2)-T-7MeW-K(Ac)-Pen(3)- |
| | TMAPF-2Nal-THP-K(NMeac)-N-3Pya-NMeK(PEG2PEG2gEC14)-CONH2 |
| 348 | cPEG3aCO-k(PEG2PEG2gEC12)-Pen(3)-N(N(Me)2)-T-7MeW-K(Ac)-Pen(3)- |
| | TMAPF-2Nal-THP-K(NMeac)-N-3Pya-NMeK(PEG2PEG2gEC12)-CONH2 |
| 350 | C14gEPEG2PEG2CO-Pen(3)-N(N(Me)2)-T-7MeW-K(Ac)-Pen(3)-TMAPF-2Nal- |
| | THP-K(NMeac)-N-3Pya-NMeK(PEG2PEG2gEC14)-CONH2 |
| 351 | cPEG3aCO-Pen(3)-N(N(Me)2)-T-7MeW-K(Ac)-Pen(3)-TMAPF-2Nal-THP- |
| | K(PEG2PEG2gEC14)-N-3Pya-NMeK(PEG2PEG2gEC14)-CONH2 |
| 352 | cPEG3aCO-Pen(3)-N(N(Me)2)-T-7MeW-K(PEG2PEG2gEC12)-Pen(3)-TMAPF- |
| | 2Nal-THP-K(NMeac)-N-3Pya-NMeK(PEG2PEG2gEC12)-CONH2 |
| 353 | cPEG3aCO-Pen(3)-N(N(Me)2)-T-7MeW-K(Ac)-Pen(3)-TMAPF-2Nal-THP- |
| | K(PEG2PEG2gEC12)-N-3Pya-NMeK(PEG2PEG2gEC12)-CONH2 |
| 354 | C12gEPEG2PEG2CO-Pen(3)-N(N(Me)2)-T-7MeW-K(Ac)-Pen(3)-TMAPF-2Nal- |
| | THP-K(NMeac)-N-3Pya-NMeK(PEG2PEG2gEC12)-CONH2 |
| 355 | cPEG3aCO-Pen(3)-N(N(Me)2)-T-7MeW-K(PEG2PEG2gEC14)-Pen(3)-TMAPF- |
| | 2Nal-THP-K(NMeac)-N-3Pya-NMeK(PEG2PEG2gEC14)-CONH2 |
| 358 | cPEG3aCO-Pen(3)-N(N(Me)2)-t-7MeW-Dab(NMeAc)-Pen(3)-TMAPF-2Nal- |
| | THP-K(NMeAc)-N-3Pya-Sar-CON(Me)2 |
| 359 | cPEG3aCO-Pen(3)-N(N(Me)2)-T-7MeW-K(Ac)-Pen(3)-TMAPF-2Nal-THP- |
| | K(NMeac)-N-3Pya-NMeK(PEG2PEG2C14)-CONH2 |
| 360 | cPEG3aCO-Pen(3)-N(N(Me)2)-T-7MeW-K(Ac)-Pen(3)-TMAPF-2Nal-THP- |
| | K(NMeac)-N-3Pya-NMeK(PEG2PEG6gEC12)-CONH2 |
| 361 | cPEG3aCO-Pen(3)-N(N(Me)2)-T-7MeW-K(Ac)-Pen(3)-TMAPF-2Nal-THP- |
| | K(NMeac)-N-3Pya-NMeK(PEG2PEG6gEC14)-CONH2 |
| 362 | cPEG3aCO-Pen(3)-N(N(Me)2)-t-7MeW-Dab(NMeAc)-Pen(3)-TMAPF-2Nal- |
| | THP-K(NMeAc)-N-3Pya-Sar-CONH2 |
| 363 | cPEG3aCO-Pen(3)-N(N(Me)2)-T-7MeW-K(NMeAc)-Pen(3)-APEG3F-2Nal- |
| | THP-K(NMeAc)-N-3Pya-Sar-CONH2 |
| 366 | C12gEPEG2PEG2CO-APEG2Ser(S*)-Pen(3)-N(N(Me)2)-T-7MeW-K(NMeAc)- |
| | Pen(3)-APEG3F-2Nal-THP-K(NMeAc)-N-3Pya-Sar-CON(Me)2 |
| 367 | C14gEPEG2PEG2CO-APEG2Ser(S*)-Pen(3)-N(N(Me)2)-T-7MeW-K(NMeAc)- |
| | Pen(3)-APEG3F-2Nal-THP-K(NMeAc)-N-3Pya-Sar-CON(Me)2 |
| 368 | C12gEPEG2PEG2CO-APEG2Ser(S*)-Pen(3)-N(N(Me)2)-T-7MeW- |
| | K(PEG2PEG2gEC12)-Pen(3)-APEG3F-2Nal-THP-K(NMeAc)-N-3Pya-Sar- |
| | CON(Me)2 |
| 369 | C14gEPEG2PEG2CO-APEG2Ser(S*)-Pen(3)-N(N(Me)2)-T-7MeW- |
| | K(PEG2PEG2gEC14)-Pen(3)-APEG3F-2Nal-THP-K(NMeAc)-N-3Pya-Sar- |
| | CON(Me)2 |
| 370 | cPEG3aCO-k(PEG2PEG6gEC12)-Pen(3)-N(N(Me)2)-T-7MeW-K(Ac)-Pen(3)- |
| | TMAPF-2Nal-THP-K(NMeac)-N-3Pya-NMeK(PEG2PEG6gEC12)-CONH2 |
| 372 | cPEG3aCO-Pen(3)-N(N(Me)2)-T-7MeW-K(Ac)-Pen(3)-TMAPF-2Nal-THP- |
| | K(NMeac)-N-3Pya-NMeK(PEG2PEG2K(PEG2PEG2gEC12)2)-CONH2 |
| 373 | cPEG3aCO-Pen(3)-N(N(Me)2)-T-7MeW-K(Ac)-Pen(3)-TMAPF-2Nal-THP- |
| | K(PEG2PEG2PEG2gEC12))-N-3Pya-NMeK(PEG2PEG2PEG2gEC12)-CONH2 |
| 375 | C12gEPEG2PEG2CO-hk(Me)3-Pen(3)-N(N(Me)2)-T-7MeW-K(NMeAc)- |
| | Pen(3)-TMAPF-2Nal-THP-K(PEG2PEG2gEC12)-N-3Pya-Sar-CON(Me)2 |
| 376 | C14gEPEG2PEG2CO-hk(Me)3-Pen(3)-N(N(Me)2)-T-7MeW-K(NMeAc)- |
| | Pen(3)-TMAPF-2Nal-THP-K(PEG2PEG2gEC14)-N-3Pya-Sar-CON(Me)2 |
| 377 | C12gEPEG2PEG2CO-hk(Me)3-Pen(3)-N(N(Me)2)-T-7MeW-K(NMeAc)- |
| | Pen(3)-TMAPF-2Nal-K(PEG2PEG2gEC12)-N-3Pya-Sar-CON(Me)2 |
| 378 | cPEG3aCO-k(PEG2PEG2gEC12)-Pen(3)-N(N(Me)2)-T-7MeW-K(NMeAc)- |
| | Pen(3)-TMAPF-2Nal-THP-K(NMeAc)-N-3Pya-Sar-CON(Me)2 |
| 379 | cPEG3aCO-k(PEG2PEG2gEC14)-Pen(3)-N(N(Me)2)-T-7MeW-K(NMeAc)- |
| | Pen(3)-TMAPF-2Nal-THP-K(NMeAc)-N-3Pya-Sar-CON(Me)2 |
| 382 | cPEG3aCO-k(PEG2PEG2gEC12)-Pen(3)-N(N(Me)2)-T-7MeW- |
| | K(PEG2PEG2gEC12)-Pen(3)-TMAPF-2Nal-THP-K(NMeAc)-N-3Pya-Sar- |
| | CON(Me)2 |
| 384 | cPEG3aCO-Pen(3)-N(N(Me)2)-T-7MeW-K(cPEG3a)-Pen(3)-TMAPF-2Nal- |
| | THP-K(NMeAc)-N-3Pya-Sar-CON(Me)2 |
| 385 | C12gEPEG2PEG2CO-k(PEG2PEG2gEC12)-Pen(3)-N(N(Me)2)-t-7MeW- |
| | Dab(NMeAc)-Pen(3)-TMAPF-2Nal-THP-K(NMeAc)-N-3Pya-Sar-CON(Me)2 |
| 386 | C14gEPEG2PEG2CO-k(PEG2PEG2gEC14)-Pen(3)-N(N(Me)2)-t-7MeW- |
| | Dab(NMeAc)-Pen(3)-TMAPF-2Nal-THP-K(NMeAc)-N-3Pya-Sar-CON(Me)2 |
| 390 | cPEG2gCO-Pen(3)-N(N(Me)2)-T-7MeW-K(NMeAc)-Pen(3)-TMAPF-2Nal- |
| | THP-K(NMeAc)-N-3Pya-Sar-CON(Me)2 |
| 391 | cPEG3gCO-Pen(3)-N(N(Me)2)-T-7MeW-K(NMeAc)-Pen(3)-TMAPF-2Nal- |
| | THP-K(NMeAc)-N-3Pya-Sar-CON(Me)2 |
| 392 | cPEG3aCO-Pen(3)-N(N(Me)2)-T-7MeW-K(4cpg)-Pen(3)-TMAPF-2Nal-THP- |
| | K(NMeAc)-N-3Pya-Sar-CON(Me)2 |
| 394 | cPEG3aCO-k(PEG2PEG2gEC12)-Pen(3)-N(N(Me)2)-T-7MeW-K(NMeAc)- |
| | Pen(3)-TMAPF-2Nal-THP-K(PEG2PEG2gEC12)-N-3Pya-Sar-CON(Me)2 |
| 395 | cPEG3aCO-k(PEG2PEG2gEC14)-Pen(3)-N(N(Me)2)-T-7MeW-K(NMeAc)- |
| | Pen(3)-TMAPF-2Nal-THP-K(PEG2PEG2gEC14)-N-3Pya-Sar-CON(Me)2 |
| 399 | cPEG3aCO-Pen(3)-N(N(Me)2)-T-7MeW-K(PEG2PEG2gEC14)-Pen(3)-TMAPF- |
| | 2Nal-THP-K(NMeAc)-N-3Pya-Sar-CON(Me)2 |
| 400 | cPEG3aCO-Pen(3)-N(N(Me)2)-T-7MeW-K(PEG2PEG2gEC12)-Pen(3)-TMAPF- |
| | 2Nal-THP-K(PEG2PEG2gEC12)-N-3Pya-Sar-CON(Me)2 |
| 401 | cPEG3aCO-Pen(3)-N(N(Me)2)-T-7MeW-K(NMeAc)-Pen(3)-TMAPF-2Nal- |
| | THP-K(PEG2PEG2gEC12)-N-3Pya-Sar-CON(Me)2 |
| 402 | cPEG3aCO-Pen(3)-N(N(Me)2)-T-7MeW-K(NMeAc)-Pen(3)-TMAPF-2Nal- |
| | THP-K(PEG2PEG2gEC14)-N-3Pya-Sar-CON(Me)2 |
| 403 | 4cpgCO-Pen(3)-N(N(Me)2)-T-7MeW-K(NMeAc)-Pen(3)-TMAPF-2Nal-THP- |
| | K(NMeAc)-N-3Pya-Sar-CON(Me)2 |
| 404 | cPEG3aCO-k(PEG2PEG2PEG2PEG2gEC12)-Pen(3)-N(N(Me)2)-T-7MeW- |
| | K(Ac)-Pen(3)-TMAPF-2Nal-THP-K(NMeac)-N-3Pya- |
| | NMeK(PEG2PEG2PEG2PEG2gEC12)-CONH2 |
| 405 | cPEG3aCO-Pen(3)-N(N(Me)2)-T-7MeW-K(NMeAc)-Pen(3)-GPEG3F-2Nal- |
| | THP-K(NMeAc)-N-3Pya-Sar-CON(Me)2 |
| 406 | cPEG3gCO-Pen(3)-N(N(Me)2)-T-7MeW-K(NMeAc)-Pen(3)-APEG3F-2Nal- |
| | THP-K(NMeAc)-N-3Pya-Sar-CON(Me)2 |
| 407 | cPEG3aCO-Pen(3)-N(N(Me)2)-T-7MeW-K(Ac)-Pen(3)-TMAPF-2Nal-THP- |
| | K(NMeac)-N-3Pya-NMeK(SP6PEG2PEG2C12)-CONH2 |
| 408 | cPEG3aCO-Pen(3)-N(N(Me)2)-T-7MeW-K(Ac)-Pen(3)-TMAPF-2Nal-THP- |
| | K(NMeac)-N-3Pya-NMeK(SP6PEG2PEG2gEC12)-CONH2 |
| 409 | cPEG3aCO-Pen(3)-N(N(Me)2)-T-7MeW-K(Ac)-Pen(3)-TMAPF-2Nal-THP- |
| | K(NMeac)-N-3Pya-NMeK(PEG2PEG2K(PEG2PEG2PEG2PEG2gEC12)2)- |
| | CONH2 |
| 410 | cPEG3aCO-Pen(3)-N(N(Me)2)-T-7MeW-K(PEG2PEG2gEC14)-Pen(3)-TMAPF- |
| | 2Nal-THP-K(PEG2PEG2gEC14)-N-3Pya-Sar-CON(Me)2 |
| 413 | cPEG2gCO-Pen(3)-N(N(Me)2)-T-7MeW-K(NMeAc)-Pen(3)-APEG3F-2Nal- |
| | THP-K(NMeAc)-N-3Pya-Sar-CON(Me)2 |
| 414 | cPEG3aCO-Pen(3)-N(N(Me)2)-t-7MeW-Dap(NMeAc)-Pen(3)-TMAPF-2Nal- |
| | THP-K(NMeAc)-N-3Pya-Sar-CON(Me)2 |
| 415 | cPEG3aCO-Pen(3)-N(N(Me)2)-T-7MeW-K(Ac)-Pen(3)-TMAPF-2Nal-THP- |
| | Dap(NMeAc)-N-3Pya-Sar-CON(Me)2 |
| 419 | cPEG3aCO-Pen(3)-N(N(Me)2)-T-7MeW-K(NMeAc)-Pen(3)-APEG3F-2Nal- |
| | THP-Dap(NMeAc)-N-3Pya-Sar-CON(Me)2 |
| 421 | cPEG3aCO-Pen(3)-N(N(me)2)-T-7(3NAcPh)W-K(NMeac)-Pen(3)-MMoEF- |
| | 2Nal-THP-K(NMeAc)-N-3Pya-Sar-CON(Me)2 |
| 466 | cPEG3aCO-Pen(3)-N(N(Me)2)-T-7MeW-K(NMeAc)-Pen(3)-APEG3F-2Nal- |
| | THP-A-N-3Pya-Sar-CON(Me)2 |
| 467 | cPEG3aCO-Pen(3)-N(N(Me)2)-T-7MeW-K(NMeAc)-Pen(3)-APEG3F-2Nal- |
| | THP-L-N-3Pya-Sar-CON(Me)2 |
| 469 | cPEG3aCO-Pen(3)-N(N(Me)2)-T-7MeW-K(NMeAc)-Pen(3)-APEG3F-2Nal- |
| | THP-F-N-3Pya-Sar-CON(Me)2 |
| 470 | cPEG3aCO-Pen(3)-N(N(Me)2)-T-7MeW-K(NMeAc)-Pen(3)-APEG3F-2Nal- |
| | THP-W-N-3Pya-Sar-CON(Me)2 |
| 472 | cPEG3aCO-Pen(3)-N(N(Me)2)-T-7MeW-K(NMeAc)-Pen(3)-Y(OTzlTMA4)- |
| | 2Nal-THP-K(NMeAc)-N-3Pya-Sar-CON(Me)2 |

TABLE 1C
Compounds

| SEQ ID | Harmonized Sequences txt |
|---|---|
| 82 | cPEG3aCO-Pen(3)-N-T-7MeW-K(Ac)-Pen(3)-AEF-2Nal-THP-E-N-3Pya-Sar- |
| | CONH2 |
| 90 | cPEG3aCO-Pen(3)-N-T-7MeW-K(Ac)-Pen(3)-AEF-2Nal-THP-K(Ac)-N-3Pya- |
| | Sar-CONH2 |
| 137 | cPEG3aCO-Pen(3)-N-T-7MeW-Q(N(Me)2)-Pen(3)-AEF-2Nal-THP- |
| | Q(N(Me)2)-N-3Pya-Sar-CONH2 |
| 139 | cPEG3aCO-Pen(3)-N(N(Me)2)-T-7MeW-Q(N(Me)2)-Pen(3)-AEF-2Nal-THP- |
| | Q(N(Me)2)-N-3Pya-Sar-CON(Me)2 |
| 142 | cPEG3aCO-Pen(3)-N(N(Me)2)-T-7MeW-AIB-Pen(3)-AEF-2Nal-THP-AIB-N- |
| | 3Pya-Sar-CON(Me)2 |
| 147 | cPEG3aCO-Pen(3)-N(N(Me)2)-T-7MeW-Q(N(Me)2)-Pen(3)-AEF-2Nal-THP- |
| | Q(N(Me)2)-N-3Pya-Sar-CONH2 |
| 149 | cPEG3aCO-Pen(3)-N(N(Me)2)-T-7MeW-K(NMeAc)-Pen(3)-AEF-2Nal-THP- |
| | K(Ac)-N-3Pya-Sar-CONH2 |
| 150 | cPEG3aCO-Pen(3)-N(N(Me)2)-T-7MeW-K(NMeAc)-Pen(3)-AEF-2Nal-THP- |
| | Q(N(Me)2)-N-3Pya-Sar-CONH2 |
| 154 | cPEG3aCO-Pen(3)-N(N(Me)2)-T-7MeW-Q(N(Me)2)-Pen(3)-AEF(N(Me)2)- |
| | 2Nal-THP-Q(N(Me)2)-N-3Pya-Sar-CON(Me)2 |
| 156 | cPEG3aCO-Pen(3)-N-T-7MeW-Q(N(Me)2)-Pen(3)-AEF-2Nal-THP- |
| | Q(N(Me)2)-N-3Pya-Sar-CON(Me)2 |
| 159 | cPEG3aCO-Pen(3)-N(N(Me)2)-T-7MeW-Q(N(Me)2)-Pen(3)-AEF-2Nal-THP- |
| | Dab(NMeAc)-N-3Pya-Sar-CON(Me)2 |
| 160 | cPEG3aCO-Pen(3)-N(N(Me)2)-t-7MeW-Dab(NMeAc)-Pen(3)-AEF-2Nal- |
| | THP-Dab(NMeAc)-N-3Pya-Sar-CON(Me)2 |
| 161 | cPEG3aCO-Pen(3)-n-T-7MeW-K(NMeAc)-Pen(3)-AEF-2Nal-THP-E-N-3Pya- |
| | Sar-CONH2 |
| 162 | cPEG3aCO-Pen(3)-n-T-7MeW-K(NMeAc)-Pen(3)-AEF-2Nal-THP-K(Ac)-N- |
| | 3Pya-Sar-CONH2 |
| 164 | cPEG3aCO-Pen(3)-N(N(Me)2)-T-7MeW-K(NMeAc)-Pen(3)-TMAPF-2Nal- |
| | THP-K(NMeAc)-N-3Pya-Sar-CON(Me)2 |
| 165 | cPEG3aCO-Pen(3)-N(N(Me)2)-T-7MeW-K(NMeAc)-Pen(3)-TMAPF-2Nal- |
| | THP-E-N-3Pya-Sar-CON(Me)2 |
| 167 | cPEG3aCO-Pen(3)-n-T-7MeW-K(NMeAc)-Pen(3)-AEF-2Nal-THP-K(NMeAc)- |
| | N-3Pya-Sar-CONH2 |
| 168 | cPEG3aCO-Pen(3)-N-T-7MeW-Q(N(Me)2)-Pen(3)-AEF-2Nal-THP- |
| | K(NMeAc)-N-3Pya-Sar-CONH2 |
| 169 | cPEG3aCO-Pen(3)-N(N(Me)2)-T-7MeW-K(NMeAc)-Pen(3)-AEF-2Nal-THP- |
| | K(NMeAc)-N-3Pya-Sar-CONH2 |
| 171 | cPEG3aCO-Pen(3)-N(N(Me)2)-t-7MeW-Dab(NMeAc)-Pen(3)-AEF-2Nal- |
| | THP-K(NMeAc)-N-3Pya-Sar-CONH2 |
| 174 | cPEG3aCO-Pen(3)-n-T-7MeW-K(NMeAc)-Pen(3)-AEF-2Nal-THP- |
| | Dab(NMeAc)-N-3Pya-Sar-CONH2 |
| 176 | cPEG3aCO-Pen(3)-N(N(Me)2)-t-7MeW-Dab(NMeAc)-Pen(3)-AEF-2Nal- |
| | THP-E-N-3Pya-Sar-CON(Me)2 |
| 177 | cPEG3aCO-Pen(3)-N(N(Me)2)-T-7MeW-K(NMeAc)-Pen(3)-AEF-2Nal-THP- |
| | Dab(NMeAc)-N-3Pya-Sar-CON(Me)2 |
| 178 | cPEG3aCO-Pen(3)-N-T-7MeW-Q(N(Me)2)-Pen(3)-AEF-2Nal-THP- |
| | Dab(NMeAc)-N-3Pya-Sar-CONH2 |
| 179 | cPEG3aCO-Pen(3)-N(N(Me)2)-T-7MeW-K(NMeAc)-Pen(3)-AEF-2Nal- |
| | diFCpx-Dab(NMeAc)-N-3Pya-Sar-CON(Me)2 |
| 180 | cPEG3aCO-Pen(3)-N(N(Me)2)-T-7MeW-K(NMeAc)-Pen(3)-AEF-2Nal- |
| | diFCpx-Dab(NMeAc)-N-3Pya-Sar-CON(Me)2 |
| 181 | cPEG3aCO-Pen(3)-N(N(Me)2)-T-7MeW-K(NMeAc)-Pen(3)-APEG3F-2Nal- |
| | THP-K(NMeAc)-N-3Pya-Sar-CON(Me)2 |
| 184 | cPEG3aCO-Pen(3)-N(N(Me)2)-T-7MeW-K(NMeAc)-Pen(3)-APEG3F-2Nal- |
| | THP-E-N-3Pya-Sar-CON(Me)2 |
| 187 | cPEG3aCO-Pen(3)-N(N(Me)2)-T-7MeW-K(NMeAc)-Pen(3)-4DMPEF-2Nal- |
| | THP-K(NMeAc)-N-3Pya-Sar-CON(Me)2 |
| 188 | cPEG3aCO-Pen(3)-n-T-7MeW-K(NMeAc)-Pen(3)-TMAPF-2Nal-THP- |
| | K(NMeAc)-N-3Pya-Sar-CONH2 |
| 191 | cPEG3aCO-Pen(3)-N(N(Me)2)-t-7MeW-Dab(NMeAc)-Pen(3)-AEF-2Nal- |
| | THP-Dab(NMeAc)-N-3Pya-Sar-CONH(PEG3a) |
| 193 | cPEG3aCO-Pen(3)-N(N(Me)2)-T-7MeW-K(NMeAc)-Pen(3)-4DMPzEF-2Nal- |
| | THP-K(NMeAc)-N-3Pya-Sar-CON(Me)2 |
| 195 | cPEG3aCO-Pen(3)-N-T-7MeW-K(Ac)-Pen(3)-APEG3F-2Nal-THP-K(Ac)-N- |
| | 3Pya-Sar-CONH2 |
| 196 | cPEG3aCO-Pen(3)-N(N(Me)2)-T-7MeW-Dab(NMecPEG3a)-Pen(3)-AEF- |
| | 2 Nal-THP-Dab(NMeAc)-N-3Pya-Sar-CON(Me)2 |
| 198 | cPEG3aCO-Pen(3)-N(N(Me)2)-T-7MeW-K(NMeac)-Pen(3)-MMoEF-2Nal- |
| | THP-K(NMeAc)-N-3Pya-Sar-CON(Me)2 |
| 199 | cPEG3aCO-Pen(3)-N(N(Me)2)-T-7MeW-K(NMeAc)-Pen(3)-APEG2F-2Nal- |
| | THP-K(NMeAc)-N-3Pya-Sar-CON(Me)2 |
| 201 | cPEG3aCO-Pen(3)-n-T-7MeW-K(NMeAc)-Pen(3)-APEG3F-2Nal-THP- |
| | K(NMeAc)-N-3Pya-Sar-CONH2 |
| 202 | cPEG3aCO-Pen(3)-N(N(Me)2)-T-7MeW-K(NMeAc)-Pen(3)-DMPMF-2Nal- |
| | THP-K(NMeAc)-N-3Pya-Sar-CON(Me)2 |
| 203 | cPEG3aCO-Pen(3)-N(N(Me)2)-T-7MeW-K(NMeAc)-Pen(3)-AEF(BisMEP))- |
| | 2Nal-THP-K(NMeAc)-N-3Pya-Sar-CON(Me)2 |
| 206 | cPEG3aCO-Pen(3)-N(N(Me)2)-T-7MeW-K(NMeAc)-Pen(3)-AEF(NsCh)-2Nal- |
| | THP-K(NMeAc)-N-3Pya-Sar-CON(Me)2 |
| 207 | cPEG3aCO-Pen(3)-N(N(Me)2)-T-7MeW-K(NMeAc)-Pen(3)-AEF(AcCh)-2Nal- |
| | THP-K(NMeAc)-N-3Pya-Sar-CON(Me)2 |
| 208 | cPEG3aCO-Pen(3)-N(N(Me)2)-T-7MeW-K(NMeAc)-Pen(3)- |
| | AEF((Ch)cPEG3a)-2Nal-THP-K(NMeAc)-N-3Pya-Sar-CON(Me)2 |
| 209 | cPEG3aCO-Pen(3)-N(N(Me)2)-T-7MeW-K(NMeAc)-Pen(3)-APEG3F-2Nal- |
| | THP-Dab(NMeAc)-N-3Pya-Sar-CON(Me)2 |
| 210 | cPEG3aCO-Pen(3)-N(N(Me)2)-T-7MeW-K(NMeAc)-Pen(3)-ACHMF-2Nal- |
| | THP-K(NMeAc)-N-3Pya-Sar-CON(Me)2 |
| 211 | cPEG3aCo-Pen(3)-N(NMe2)-T-7MeW-K(NMeAc)-Pen(3)-AEF(NHCh)-2Nal- |
| | THP-K(NMeAc)-N-3Pya-Sar-CON(Me)2 |
| 212 | cPEG3aCO-Pen(3)-N(N(Me)2)-T-7MeW-K(NMeAc)-Pen(3)-APEG3F-2Nal- |
| | THP-Dab(NMeAc)-N-3Pya-Sar-CON(Me)2 |
| 215 | cPEG3aCO-Pen(3)-N(N(Me)2)-T-7MeW-K(NMeAc)-Pen(3)-DMMMF-2Nal- |
| | THP-K(NMeAc)-N-3Pya-Sar-CON(Me)2 |
| 220 | cPEG3aCO-Pen(3)-N(N(Me)2)-T-7MeW-K(NMeAc)-Pen(3)-TMAPF-2Nal- |
| | THP-K(NMeAc)-N-3Pya-Sar-CO(NHPEG3a) |
| 221 | cPEG3aCO-Pen(3)-N(N(Me)2)-T-7MeW-K(NMeAc)-Pen(3)-AEF(BisMEPa)- |
| | 2Nal-THP-K(NMeAc)-N-3Pya-Sar-CON(Me)2Â |
| 222 | cPEG3aCO-Pen(3)-N(N(Me)2)-T-7MeW-K(NMeAc)-Pen(3)-ACHMF-2Nal- |
| | THP-K(NMeAc)-N-3Pya-Sar-CON(Me)2 |
| 223 | cPEG3aCO-Pen(3)-N(N(Me)2)-T-7MeW-K(NMeAc)-Pen(3)-MMPEG3F-2Nal- |
| | THP-K(NMeAc)-N-3Pya-Sar-CON(Me)2 |
| 224 | cPEG3aCO-Pen(3)-N(N(Me)2)-T-7MeW-K(NMeAc)-Pen(3)-MPzPEG3F- |
| | 2Nal-THP-K(NMeAc)-N-3Pya-Sar-CON(Me)2 |
| 226 | cPEG3aCO-Pen(3)-N(N(Me)2)-T-7MeW-K(NMeAc)-Pen(3)-TMA4F-2Nal- |
| | THP-K(NMeAc)-N-3Pya-Sar-CON(Me)2 |
| 227 | cPEG3aCO-Pen(3)-N(N(Me)2)-T-7MeW-K(NMeAc)-Pen(3)-mPEG2TMA4F- |
| | 2Nal-THP-K(NMeAc)-N-3Pya-Sar-CON(Me)2 |
| 228 | cPEG3aCO-Pen(3)-N(N(Me)2)-T-7MeW-K(NMeAc)-Pen(3)-mPEG2TMA2F- |
| | 2Nal-THP-K(NMeAc)-N-3Pya-Sar-CON(Me)2 |
| 229 | cPEG3aCO-Pen(3)-N(N(Me)2)-T-7MeW-K(NMeAc)-Pen(3)-TMA3F-2Nal- |
| | THP-K(NMeAc)-N-3Pya-Sar-CON(Me)2 |
| 230 | cPEG3aCO-Pen(3)-N(N(Me)2)-T-7MeW-K(NMeAc)-Pen(3)-TMA3F-2Nal- |
| | THP-K(NMeAc)-N-3Pya-Sar-CON(Me)2 |
| 231 | cPEG3aCO-Pen(3)-N(N(Me)2)-T-7MeW-K(NMeac)-Pen(3)-NPyEF-2Nal- |
| | THP-K(NMeAc)-N-3Pya-Sar-CON(Me)2 |
| 233 | cPEG3aCO-Pen(3)-N(N(Me)2)-T-7MeW-K(NMeAc)-Pen(3)-APEG3F-2Nal- |
| | THP-K(Ac)-N-3Pya-Sar-CON(Me)2 |
| 234 | cPEG3aCO-Pen(3)-N(N(Me)2)-T-7MeW-K(NMeAc)-Pen(3)-AEF(MePrpa)- |
| | 2Nal-THP-K(NMeAc)-N-3Pya-Sar-CON(Me)2 |
| 236 | cPEG3aCO-Pen(3)-N(N(Me)2)-T-7MeW-K(NMeAc)-Pen(3)-Y(C9OH)-2Nal- |
| | THP-K(NMeAc)-N-3Pya-Sar-CON(Me)2 |
| 237 | cPEG3aCO-Pen(3)-N(N(Me)2)-T-7MeW-K(NMeAc)-Pen(3)- |
| | AEF(NMePEG2a)-2Nal-THP-K(NMeAc)-N-3Pya-Sar-CON(Me)2 |
| 238 | cPEG3aCO-Pen(3)-N(N(Me)2)-T-7MeW-K(NMeAc)-Pen(3)-AEF(PEG2a)- |
| | 2Nal-THP-K(NMeAc)-N-3Pya-Sar-CON(Me)2 |
| 239 | cPEG3aCO-Pen(3)-N(N(Me)2)-T-7MeW-K(NMeAc)-pen(3)- |
| | AEF(NMe2mPEG3)-2Nal-THP-K(NMeAc)-N-3Pya-Sar-CON(Me)2 |
| 240 | cPEG3aCO-Pen(3)-n-T-7MeW-K(NMeAc)-Pen(3)-APEG3F-2Nal-THP- |
| | K(NMeAc)-N-3Pya-Sar-CON(Me)2 |
| 241 | cPEG3aCO-Pen(3)-N(N(Me)2)-T-7MeW-K(NMeAc)-Pen(3)- |
| | AEF(NMeBismPEG3)-2Nal-THP-K(NMeAc)-N-3Pya-Sar-CON(Me)2 |
| 242 | cPEG3aCO-Pen(3)-N(N(Me)2)-T-7MeW-K(NMeAc)-Pen(3)-YC8COPip-2Nal- |
| | THP-K(NMeAc)-N-3Pya-Sar-CON(Me)2 |
| 243 | cPEG3aCO-Pen(3)-N(N(Me)2)-T-7MeW-K(Ac)-Pen(3)-APEG3F-2Nal-THP- |
| | K(NMeAc)-N-3Pya-Sar-CON(Me)2 |
| 245 | cPEG3aCO-Pen(3)-N(N(Me)2)-T-7MeW-APEG2Ser-Pen(3)-AEF-2Nal-THP- |
| | K(NMeAc)-N-3Pya-Sar-CON(Me)2 |
| 246 | cPEG3aCO-Pen(3)-N(N(Me)2)-T-7MeW-APEG2Ser(R*)-Pen(3)-AEF-2Nal- |
| | THP-K(NMeAc)-N-3Pya-Sar-CON(Me)2 |
| 247 | cPEG3aCO-Pen(3)-N(N(Me)2)-T-7MeW-APEG2Ser(S*)-Pen(3)-TMAPF- |
| | 2Nal-THP-K(NMeAc)-N-3Pya-Sar-CON(Me)2 |
| 248 | cPEG3aCO-Pen(3)-N(N(Me)2)-T-7MeW-APEG2Ser(R*)-Pen(3)-TMAPF- |
| | 2Nal-THP-K(NMeAc)-N-3Pya-Sar-CON(Me)2 |
| 254 | cPEG3aCO-Pen(3)-N(N(Me)2)-T-7MeW-K(NMeAc)-Pen(3)- |
| | YC8CO(NHPEG3a)-2Nal-THP-K(NMeAc)-N-3Pya-Sar-CON(Me)2 |
| 261 | cPEG3aCO-Pen(3)-N(N(Me)2)-T-7MeW-K(NMeAc)-pen(3)-AEF(NMe3)- |
| | 2Nal-THP-K(NMeAc)-N-3Pya-Sar-CON(Me)2 |
| 262 | cPEG3aCO-Pen(3)-N(N(Me)2)-T-7MeW-K(NMeAc)-Pen(3)-MTASF-2Nal- |
| | THP-K(NMeAc)-N-3Pya-Sar-CON(Me)2 |
| 263 | cPEG3aCO-Pen(3)-N(N(Me)2)-T-7MeW-K(NMeAc)-Pen(3)-TBAPEG3F-2Nal- |
| | THP-K(NMeAc)-N-3Pya-Sar-CON(Me)2 |
| 264 | cPEG3aCO-Pen(3)-N(N(Me)2)-T-7MeW-K(NMeAc)-Pen(3)-AEF(aPEG2a)- |
| | 2Nal-THP-K(NMeAc)-N-3Pya-Sar-CON(Me)2 |
| 265 | cPEG3aCO-Pen(3)-N(N(Me)2)-T-7MeW-K(NMeAc)-Pen(3)- |
| | AEF(BisPEG2a)(RS)-2Nal-THP-K(NMeAc)-N-3Pya-Sar-CON(Me)2 |
| 266 | cPEG3aCO-Pen(3)-N(N(Me)2)-T-7MeW-K(NMeAc)-Pen(3)- |
| | AEF(BisPEG2a)(S*)-2Nal-THP-K(NMeAc)-N-3Pya-Sar-CON(Me)2 |
| 269 | cPEG3aCO-Pen(3)-N(N(Me)2)-T-7MeW-K(NMeAc)-Pen(3)-DMTASF-2Nal- |
| | THP-K(NMeAc)-N-3Pya-Sar-CON(Me)2 |
| 271 | cPEG3aCO-Pen(3)-N(N(Me)2)-T-7MeW-K(NMeAc)-Pen(3)-morfTMA4F- |
| | 2Nal-THP-K(NMeAc)-N-3Pya-Sar-CON(Me)2 |
| 272 | cPEG3aCO-Pen(3)-N(N(Me)2)-T-7MeW-K(NMeAc)-Pen(3)-mPEG3TMA4F- |
| | 2Nal-THP-K(NMeAc)-N-3Pya-Sar-CON(Me)2 |
| 273 | cPEG3aCO-Pen(3)-N(N(Me)2)-T-7MeW-K(NMeAc)-Pen(3)- |
| | PiperazinequatF-2Nal-THP-K(NMeAc)-N-3Pya-Sar-CON(Me)2 |
| 274 | cPEG3aCO-Pen(3)-N(N(Me)2)-T-7MeW-K(NMeac)-Pen(3)-MMoPF-2Nal- |
| | THP-K(NMeAc)-N-3Pya-Sar-CON(Me)2 |
| 277 | cPEG3aCO-Pen(3)-N(N(Me)2)-T-7MeW-K(NMeAc)-Pen(3)-AEF(SPD)-2Nal- |
| | THP-K(NMeAc)-N-3Pya-Sar-CON(Me)2 |
| 283 | cPEG3aCO-Pen(3)-N(N(Me)2)-T-7MeW-K(NMeAc)-Pen(3)- |
| | Y(OTzlChmPEG3)-2Nal-THP-K(NMeAc)-N-3Pya-Sar-CON(Me)2 |
| 284 | cPEG3aCO-Pen(3)-N(N(Me)2)-T-7MeW-K(NMeAc)-Pen(3)-Y(OTzlTMA4)- |
| | 2Nal-THP-K(NMeAc)-N-3Pya-Sar-CON(Me)2 |
| 285 | cPEG3aCO-Pen(3)-N(N(Me)2)-T-7MeW-K(NMeAc)-Pen(3)-Y(OTzlChmPEG)- |
| | 2Nal-THP-K(NMeAc)-N-3Pya-Sar-CON(Me)2 |
| 286 | cPEG3aCO-Pen(3)-N(N(Me)2)-T-7MeW-K(NMeAc)-Pen(3)-Y(OTzlCh)-2Nal- |
| | THP-K(NMeAc)-N-3Pya-Sar-CON(Me)2 |
| 287 | cPEG3aCO-Pen(3)-N(N(Me)2)-T-7MeW-K(NMeAc)-Pen(3)-Tzl(Ch)-2Nal- |
| | THP-K(NMeAc)-N-3Pya-Sar-CON(Me)2 |
| 288 | cPEG3aCO-Pen(3)-N(N(Me)2)-T-7MeW-K(NMeAc)-Pen(3)-TzlChmPEG- |
| | 2Nal-THP-K(NMeAc)-N-3Pya-Sar-CON(Me)2 |
| 289 | cPEG3aCO-Pen(3)-N(N(Me)2)-T-7MeW-K(NMeAc)-Pen(3)-TzlChmPEG3- |
| | 2Nal-THP-K(NMeAc)-N-3Pya-Sar-CON(Me)2 |
| 290 | cPEG3aCO-Pen(3)-N(N(Me)2)-T-7MeW-K(NMeAc)-Pen(3)-Y(OtzlC1aC8)- |
| | 2Nal-THP-K(NMeAc)-N-3Pya-Sar-CON(Me)2 |
| 291 | cPEG3aCO-Pen(3)-N(N(Me)2)-T-7MeW-K(NMeAc)-Pen(3)-Y(OTzlChC16)- |
| | 2Nal-THP-K(NMeAc)-N-3Pya-Sar-CON(Me)2 |
| 292 | cPEG3aCO-Pen(3)-N(N(Me)2)-T-7MeW-K(NMeAc)-Pen(3)- |
| | Diazabiclyclooctane6F-2Nal-THP-K(NMeAc)-N-3Pya-Sar-CON(Me)2 |
| 293 | cPEG3aCO-Pen(3)-N(N(Me)2)-T-7MeW-K(NMeAc)-Pen(3)-TMA6F-2Nal- |
| | THP-K(NMeAc)-N-3Pya-Sar-CON(Me)2 |
| 294 | cPEG3aCO-Pen(3)-N(N(me)2)-T-7(3NAcPh)W-K(NMeAc)-Pen(3)-TMAPF- |
| | 2Nal-THP-K(NMeAc)-N-3Pya-Sar-CON(Me)2 |
| 295 | cPEG3aCO-Pen(3)-N(N(Me)2)-T-7MeW-K(NMeAc)-Pen(3)-TMA8F-2Nal- |
| | THP-K(NMeAc)-N-3Pya-Sar-CON(Me)2 |
| 297 | cPEG3aCO-Pen(3)-N(N(Me)2)-T-7MeW-K(NMeAc)-Pen(3)-NPyPEG3F-2Nal- |
| | THP-K(NMeAc)-N-3Pya-Sar-CON(Me)2 |
| 298 | cPEG3aCO-Pen(3)-N(N(Me)2)-T-7MeW-K(NMeAc)-Pen(3)-dFPPEG3F-2Nal- |
| | THP-K(NMeAc)-N-3Pya-Sar-CON(Me)2 |
| 299 | cPEG3aCO-Pen(3)-n-T-7MeW-K(NMeAc)-Pen(3)-TMAPF-2Nal-THP- |
| | K(NMeAc)-N-3Pya-Sar-CON(Me)2 |
| 300 | cPEG3aCO-Pen(3)-N(N(Me))-T-7MeW-K(NMeAc)-Pen(3)-TMAPF-2Nal-THP- |
| | K(NMeAc)-N-3Pya-Sar-CON(Me)2 |
| 301 | cPEG3aCO-Pen(3)-N(N(Me)2)-T-7MeW-K(NMeAC)-Pen(3)-TMAPF-2Nal- |
| | THP-Dab(NMeAc)-N-3Pya-Sar-CON(Me)2 |
| 302 | cPEG3aCO-Pen(3)-N(N(Me)2)-T-7MeW-K(NMeAc)-Pen(3)-F(4TzlTMA4)- |
| | 2Nal-THP-K(NMeAc)-N-3Pya-Sar-CON(Me)2 |
| 303 | cPEG3aCO-Pen(3)-N(N(Me)2)-T-7MeW-K(NMeAc)-Pen(3)-F(4TzlMME)- |
| | 2Nal-THP-K(NMeAc)-N-3Pya-Sar-CON(Me)2 |
| 304 | cPEG3aCO-Pen(3)-N(N(Me)2)-T-7MeW-K(NMeAc)-Pen(3)-F(4G)-2Nal-THP- |
| | K(NMeAc)-N-3Pya-Sar-CON(Me)2 |
| 305 | cPEG3aCO-Pen(3)-N(N(Me)2)-T-7MeW-K(NMeAc)-Pen(3)-Y(OEOXlMECh)- |
| | 2Nal-THP-K(NMeAc)-N-3Pya-Sar-CON(Me)2 |
| 306 | cPEG3aCO-Pen(3)-N(N(Me)2)-T-7MeW-K(NMeAc)-Pen(3)-Y(OZOXlMECh)- |
| | 2Nal-THP-K(NMeAc)-N-3Pya-Sar-CON(Me)2 |
| 307 | cPEG3aCO-Pen(3)-N(N(Me)2)-T-7MeW-K(NMeAC)-Pen(3)-TMAPF-2Nal- |
| | THP-K(NMeaC)-N-3Pya-NMeK(PEG2PEG2C12)-CONH2 |
| 308 | cPEG3aCO-Pen(3)-N(N(Me)2)-T-7MeW-K(Ac)-Pen(3)-TMAPF-2Nal-THP- |
| | Dab(NMeAc)-N-3Pya-Sar-CON(Me)2 |
| 309 | cPEG3aCO-Pen(3)-n-T-7MeW-K(NMeAC)-Pen(3)-TMAPF-2Nal-THP- |
| | Dab(NMeAc)-N-3Pya-Sar-CON(Me)2 |
| 310 | cPEG3aCO-Pen(3)-N(N(Me)2)-T-7MeW-K(NMeAC)-Pen(3)-TMAPF-2Nal- |
| | THP-Q(N(Me)2)-N-3Pya-Sar-CON(Me)2 |
| 311 | cPEG3aCO-Pen(3)-N-t-7MeW-Dab(NMeAc)-Pen(3)-TMAPF-2Nal-THP- |
| | K(NMeAC)-N-3Pya-Sar-CON(Me)2 |
| 312 | cPEG3aCO-Pen(3)-N(N(Me)2)-T-7MeW-Q(N(Me)2)-Pen(3)-TMAPF-2Nal- |
| | THP-K(NMeAC)-N-3Pya-Sar-CON(Me)2 |
| 313 | cPEG3aCO-Pen(3)-N(N(Me)2)-T-7MeW-K(Ac)-Pen(3)-TMAPF-2Nal-THP- |
| | K(NMeaC)-N-3Pya-NMeK(PEG2PEG2gEC12)-CONH2 |
| 314 | cPEG3aCO-Pen(3)-N(N(Me)2)-T-7MeW-K(NMeAC)-Pen(3)-TMAPF-2Nal- |
| | THP-K(NMeaC)-N-3Pya-NMeK(PEG2PEG2gEC12)-CONH2 |
| 315 | cPEG3aCO-Pen(3)-N(N(Me)2)-T-7MeW-K(NMeAC)-Pen(3)-TMAPF-2Nal- |
| | THP-K(NMeaC)-N-3Pya-NMeK(PEG2PEG2gEC14)-CONH2 |
| 316 | cPEG3aCO-Pen(3)-N(N(Me)2)-T-7MeW-K(NMeAc)-Pen(3)-TMAPF-2Nal- |
| | THP-A-N-3Pya-Sar-CON(Me)2 |
| 317 | cPEG3aCO-Pen(3)-N-T-7MeW-K(Ac)-Pen(3)-TMAPF-2Nal-THP-A-N-3Pya- |
| | Sar-CON(Me)2 |
| 320 | cPEG3aCO-Pen(3)-N(N(Me)2)-T-7MeW-K(NMeAc)-Pen(3)-AEF-2Nal-THP- |
| | K(NMeAc)-N-3Pya-Sar-CON(Me)2 |
| 321 | cPEG3aCO-Pen(3)-N(N(Me)2)-T-7MeW-K(NMeAc)-Pen(3)-AEF(N(Me)2)- |
| | 2Nal-THP-K(NMeAc)-N-3Pya-Sar-CON(Me)2 |
| 326 | cPEG3aCO-Pen(3)-N(N(Me)2)-T-7MeW-K(NMeAc)-Pen(3)-TMAPF-2Nal- |
| | THP-K(NMeAc)-N-3Pya-Sar-CONH2 |
| 327 | cPEG3aCO-Pen(3)-N(N(Me)2)-T-7MeW-K(NMeAc)-Pen(3)-TMAPF-2Nal- |
| | THP-Cit-N-3Pya-Sar-CON(Me)2 |
| 328 | cPEG3aCO-Pen(3)-K(Me)3-T-7MeW-K(NMeAc)-Pen(3)-TMAPF-2Nal-THP- |
| | K(NMeAc)-N-3Pya-Sar-CON(Me)2 |
| 329 | cPEG3aCO-Pen(3)-N(N(Me)2)-T-7MeW-K(Me)3-Pen(3)-TMAPF-2Nal-THP- |
| | K(NMeAc)-N-3Pya-Sar-CON(Me)2 |
| 330 | cPEG3aCO-Pen(3)-N(N(Me)2)-T-7MeW-K(NMeAc)-Pen(3)-TMAPF-2nal- |
| | Acpx-K(NMeAc)-N-3Pya-Sar-CON(Me)2 |
| 331 | cPEG3aCO-Pen(3)-N(N(Me)2)-T-7MeW-K(NMeAc)-Pen(3)-F(4N3)-2Nal- |
| | THP-K(NMeAc)-N-3Pya-Sar-CON(Me)2 |
| 332 | cPEG3aCO-Pen(3)-N(N(Me)2)-T-7MeW-K(NMeAc)-Pen(3)-F(4TzlTMA1)- |
| | 2Nal-THP-K(NMeAc)-N-3Pya-Sar-CON(Me)2 |
| 333 | cPEG3aCO-Pen(3)-N(N(Me)2)-T-7MeW-K(NMeAc)-Pen(3)-F(4TzlTMA2)- |
| | 2Nal-THP-K(NMeAc)-N-3Pya-Sar-CON(Me)2 |
| 334 | cPEG3aCO-Pen(3)-N(N(Me)2)-T-7MeW-K(NMeAc)-Pen(3)-F(4TzlTMA3)- |
| | 2Nal-THP-K(NMeAc)-N-3Pya-Sar-CON(Me)2 |
| 335 | cPEG3aCO-Pen(3)-N(N(Me)2)-T-7MeW-K(NMeAc)-Pen(3)-F(4TzlTMA5)- |
| | 2Nal-THP-K(NMeAc)-N-3Pya-Sar-CON(Me)2 |
| 336 | cPEG3aCO-Pen(3)-N(N(Me)2)-T-7MeW-K(NMeAc)-Pen(3)-F(4TzlMMo4)- |
| | 2Nal-THP-K(NMeAc)-N-3Pya-Sar-CON(Me)2 |
| 337 | cPEG3aCO-Pen(3)-N(N(Me)2)-T-7MeW-K(NMeAc)-Pen(3)- |
| | F(4TzlDMA4mPEG)-2Nal-THP-K(NMeAc)-N-3Pya-Sar-CON(Me)2 |
| 338 | cPEG3aCO-Pen(3)-N(N(Me)2)-T-7MeW-K(NMeAc)-Pen(3)-hFTMAPF-2Nal- |
| | THP-K(NMeAc)-N-3Pya-Sar-CON(Me)2 |
| 339 | cPEG3aCO-Pen(3)-N(N(Me)2)-T-7MeW-K(NMeAc)-Pen(3)-TMAPF-2nal- |
| | Achx-K(NMeAc)-N-3Pya-Sar-CON(Me)2 |
| 340 | cPEG3aCO-Pen(3)-N(N(Me)2)-T-7MeW-K(NMeAc)-Pen(3)-TMAPF-2Nal- |
| | Chg-K(NMeAc)-N-3Pya-Sar-CON(Me)2 |
| 341 | cPEG3aCO-Pen(3)-N(N(Me)2)-T-7MeW-K(NMeAc)-Pen(3)-TMAPF-2Nal- |
| | Aib-K(NMeAc)-N-3Pya-Sar-CON(Me)2 |
| 342 | cPEG3aCO-Pen(3)-K(Me)3-T-7MeW-K(NMeAc)-Pen(3)-TMAPF-2nal-Achx- |
| | K(NMeAc)-N-3Pya-Sar-CON(Me)2 |
| 346 | cPEG3aCO-Pen(3)-N(N(Me)2)-T-7MeW-Cit-Pen(3)-TMAPF-2Nal-THP- |
| | K(NMeAc)-N-3Pya-Sar-CON(Me)2 |
| 347 | cPEG3aCO-k(PEG2PEG2gEC14)-Pen(3)-N(N(Me)2)-T-7MeW-K(Ac)-Pen(3)- |
| | TMAPF-2Nal-THP-K(NMeac)-N-3Pya-NMeK(PEG2PEG2gEC14)-CONH2 |
| 348 | cPEG3aCO-k(PEG2PEG2gEC12)-Pen(3)-N(N(Me)2)-T-7MeW-K(Ac)-Pen(3)- |
| | TMAPF-2Nal-THP-K(NMeac)-N-3Pya-NMeK(PEG2PEG2gEC12)-CONH2 |
| 349 | cPEG3aCO-Pen(3)-N-T-7MeW-K(Ac)-Pen(3)-TMAPF-2Nal-THP- |
| | K(PEG2PEG2gEC12)-N-3Pya-NMeK(PEG2PEG2gEC12)-CONH2 |
| 351 | cPEG3aCO-Pen(3)-N(N(Me)2)-T-7MeW-K(Ac)-Pen(3)-TMAPF-2Nal-THP- |
| | K(PEG2PEG2gEC14)-N-3Pya-NMeK(PEG2PEG2gEC14)-CONH2 |
| 352 | cPEG3aCO-Pen(3)-N(N(Me)2)-T-7MeW-K(PEG2PEG2gEC12)-Pen(3)- |
| | TMAPF-2Nal-THP-K(NMeac)-N-3Pya-NMeK(PEG2PEG2gEC12)-CONH2 |
| 353 | cPEG3aCO-Pen(3)-N(N(Me)2)-T-7MeW-K(Ac)-Pen(3)-TMAPF-2Nal-THP- |
| | K(PEG2PEG2gEC12)-N-3Pya-NMeK(PEG2PEG2gEC12)-CONH2 |
| 355 | cPEG3aCO-Pen(3)-N(N(Me)2)-T-7MeW-K(PEG2PEG2gEC14)-Pen(3)- |
| | TMAPF-2Nal-THP-K(NMeac)-N-3Pya-NMeK(PEG2PEG2gEC14)-CONH2 |
| 356 | cPEG3aCO-Pen(3)-K(PEG2PEG2gEC12)-T-7MeW-K(Ac)-Pen(3)-TMAPF- |
| | 2Nal-THP-K(NMeac)-N-3Pya-NMeK(PEG2PEG2gEC12)-CONH2 |
| 357 | cPEG3aCO-Pen(3)-K(PEG2PEG2gEC14)-T-7MeW-K(Ac)-Pen(3)-TMAPF- |
| | 2Nal-THP-K(NMeac)-N-3Pya-NMeK(PEG2PEG2gEC14)-CONH2 |
| 358 | cPEG3aCO-Pen(3)-N(N(Me)2)-t-7MeW-Dab(NMeAc)-Pen(3)-TMAPF-2Nal- |
| | THP-K(NMeAc)-N-3Pya-Sar-CON(Me)2 |
| 359 | cPEG3aCO-Pen(3)-N(N(Me)2)-T-7MeW-K(Ac)-Pen(3)-TMAPF-2Nal-THP- |
| | K(NMeac)-N-3Pya-NMeK(PEG2PEG2C14)-CONH2 |
| 360 | cPEG3aCO-Pen(3)-N(N(Me)2)-T-7MeW-K(Ac)-Pen(3)-TMAPF-2Nal-THP- |
| | K(NMeac)-N-3Pya-NMeK(PEG2PEG6gEC12)-CONH2 |
| 361 | cPEG3aCO-Pen(3)-N(N(Me)2)-T-7MeW-K(Ac)-Pen(3)-TMAPF-2Nal-THP- |
| | K(NMeac)-N-3Pya-NMeK(PEG2PEG6gEC14)-CONH2 |
| 362 | cPEG3aCO-Pen(3)-N(N(Me)2)-t-7MeW-Dab(NMeAc)-Pen(3)-TMAPF-2Nal- |
| | THP-K(NMeAc)-N-3Pya-Sar-CONH2 |
| 363 | cPEG3aCO-Pen(3)-N(N(Me)2)-T-7MeW-K(NMeAc)-Pen(3)-APEG3F-2Nal- |
| | THP-K(NMeAc)-N-3Pya-Sar-CONH2 |
| 364 | cPEG3aCO-Pen(3)-APEG2Ser(S*)-T-7MeW-K(NMeAc)-Pen(3)-APEG3F- |
| | 2Nal-THP-K(NMeAc)-N-3Pya-Sar-CON(Me)2 |
| 370 | cPEG3aCO-k(PEG2PEG6gEC12)-Pen(3)-N(N(Me)2)-T-7MeW-K(Ac)-Pen(3)- |
| | TMAPF-2Nal-THP-K(NMeac)-N-3Pya-NMeK(PEG2PEG6gEC12)-CONH2 |
| 371 | cPEG3aCO-Pen(3)-N-T-7MeW-K(Ac)-Pen(3)-TMAPF-2Nal-THP-K(NMeac)- |
| | N-3Pya-NMeK(PEG2PEG2gEC12)-CONH2 |
| 372 | cPEG3aCO-Pen(3)-N(N(Me)2)-T-7MeW-K(Ac)-Pen(3)-TMAPF-2Nal-THP- |
| | K(NMeac)-N-3Pya-NMeK(PEG2PEG2K(PEG2PEG2gEC12)2)-CONH2 |
| 373 | cPEG3aCO-Pen(3)-N(N(Me)2)-T-7MeW-K(Ac)-Pen(3)-TMAPF-2Nal-THP- |
| | K(PEG2PEG2PEG2gEC12))-N-3Pya-NMeK(PEG2PEG2PEG2gEC12)-CONH2 |
| 378 | cPEG3aCO-k(PEG2PEG2gEC12)-Pen(3)-N(N(Me)2)-T-7MeW-K(NMeAc)- |
| | Pen(3)-TMAPF-2Nal-THP-K(NMeAc)-N-3Pya-Sar-CON(Me)2 |
| 379 | cPEG3aCO-k(PEG2PEG2gEC14)-Pen(3)-N(N(Me)2)-T-7MeW-K(NMeAc)- |
| | Pen(3)-TMAPF-2Nal-THP-K(NMeAc)-N-3Pya-Sar-CON(Me)2 |
| 380 | cPEG3aCO-k(PEG2PEG2gEC12)-Pen(3)-K(PEG2PEG2gEC12)-T-7MeW- |
| | K(NMeAc)-Pen(3)-TMAPF-2Nal-THP-K(NMeAc)-N-3Pya-Sar-CON(Me)2 |
| 381 | cPEG3aCO-k(PEG2PEG2gEC14)-Pen(3)-K(PEG2PEG2gEC14)-T-7MeW- |
| | K(NMeAc)-Pen(3)-TMAPF-2Nal-THP-K(NMeAc)-N-3Pya-Sar-CON(Me)2 |
| 382 | cPEG3aCO-k(PEG2PEG2gEC12)-Pen(3)-N(N(Me)2)-T-7MeW- |
| | K(PEG2PEG2gEC12)-Pen(3)-TMAPF-2Nal-THP-K(NMeAc)-N-3Pya-Sar- |
| | CON(Me)2 |
| 384 | cPEG3aCO-Pen(3)-N(N(Me)2)-T-7MeW-K(cPEG3a)-Pen(3)-TMAPF-2Nal- |
| | THP-K(NMeAc)-N-3Pya-Sar-CON(Me)2 |
| 387 | cPEG3aCO-Pen(3)-K(PEG2PEG2gEC12)-T-7MeW-K(NMeAc)-Pen(3)-TMAPF- |
| | 2Nal-THP-K(NMeAc)-N-3Pya-Sar-CON(Me)2 |
| 388 | cPEG3aCO-Pen(3)-K(PEG2PEG2gEC14)-T-7MeW-K(NMeAc)-Pen(3)-TMAPF- |
| | 2Nal-THP-K(NMeAc)-N-3Pya-Sar-CON(Me)2 |
| 389 | cPEG3aCO-Pen(3)-K(PEG2PEG2gEC12)-T-7MeW-K(PEG2PEG2gEC12)- |
| | Pen(3)-TMAPF-2Nal-THP-K(NMeAc)-N-3Pya-Sar-CON(Me)2 |
| 392 | cPEG3aCO-Pen(3)-N(N(Me)2)-T-7MeW-K(4cpg)-Pen(3)-TMAPF-2Nal-THP- |
| | K(NMeAc)-N-3Pya-Sar-CON(Me)2 |
| 393 | cPEG3aCO-Pen(3)-K(cPEG3a)-T-7MeW-K(NMeAc)-Pen(3)-TMAPF-2Nal- |
| | THP-K(NMeAc)-N-3Pya-Sar-CON(Me)2 |
| 394 | cPEG3aCO-k(PEG2PEG2gEC12)-Pen(3)-N(N(Me)2)-T-7MeW-K(NMeAc)- |
| | Pen(3)-TMAPF-2Nal-THP-K(PEG2PEG2gEC12)-N-3Pya-Sar-CON(Me)2 |
| 395 | cPEG3aCO-k(PEG2PEG2gEC14)-Pen(3)-N(N(Me)2)-T-7MeW-K(NMeAc)- |
| | Pen(3)-TMAPF-2Nal-THP-K(PEG2PEG2gEC14)-N-3Pya-Sar-CON(Me)2 |
| 396 | cPEG3aCO-Pen(3)-K(PEG2PEG2gEC14)-T-7MeW-K(PEG2PEG2gEC14)- |
| | Pen(3)-TMAPF-2Nal-THP-K(NMeAc)-N-3Pya-Sar-CON(Me)2 |
| 397 | cPEG3aCO-Pen(3)-K(PEG2PEG2gEC12)-T-7MeW-K(NMeAc)-Pen(3)-TMAPF- |
| | 2Nal-THP-K(PEG2PEG2gEC12)-N-3Pya-Sar-CON(Me)2 |
| 398 | cPEG3aCO-Pen(3)-K(PEG2PEG2gEC14)-T-7MeW-K(NMeAc)-Pen(3)-TMAPF- |
| | 2Nal-THP-K(PEG2PEG2gEC14)-N-3Pya-Sar-CON(Me)2 |
| 399 | cPEG3aCO-Pen(3)-N(N(Me)2)-T-7MeW-K(PEG2PEG2gEC14)-Pen(3)- |
| | TMAPF-2Nal-THP-K(NMeAc)-N-3Pya-Sar-CON(Me)2 |
| 400 | cPEG3aCO-Pen(3)-N(N(Me)2)-T-7MeW-K(PEG2PEG2gEC12)-Pen(3)- |
| | TMAPF-2Nal-THP-K(PEG2PEG2gEC12)-N-3Pya-Sar-CON(Me)2 |
| 401 | cPEG3aCO-Pen(3)-N(N(Me)2)-T-7MeW-K(NMeAc)-Pen(3)-TMAPF-2Nal- |
| | THP-K(PEG2PEG2gEC12)-N-3Pya-Sar-CON(Me)2 |
| 402 | cPEG3aCO-Pen(3)-N(N(Me)2)-T-7MeW-K(NMeAc)-Pen(3)-TMAPF-2Nal- |
| | THP-K(PEG2PEG2gEC14)-N-3Pya-Sar-CON(Me)2 |
| 404 | cPEG3aCO-k(PEG2PEG2PEG2PEG2gEC12)-Pen(3)-N(N(Me)2)-T-7MeW- |
| | K(Ac)-Pen(3)-TMAPF-2Nal-THP-K(NMeac)-N-3Pya- |
| | NMeK(PEG2PEG2PEG2PEG2gEC12)-CONH2 |
| 405 | cPEG3aCO-Pen(3)-N(N(Me)2)-T-7MeW-K(NMeAc)-Pen(3)-GPEG3F-2Nal- |
| | THP-K(NMeAc)-N-3Pya-Sar-CON(Me)2 |
| 407 | cPEG3aCO-Pen(3)-N(N(Me)2)-T-7MeW-K(Ac)-Pen(3)-TMAPF-2Nal-THP- |
| | K(NMeac)-N-3Pya-NMeK(SP6PEG2PEG2C12)-CONH2 |
| 408 | cPEG3aCO-Pen(3)-N(N(Me)2)-T-7MeW-K(Ac)-Pen(3)-TMAPF-2Nal-THP- |
| | K(NMeac)-N-3Pya-NMeK(SP6PEG2PEG2gEC12)-CONH2 |
| 409 | cPEG3aCO-Pen(3)-N(N(Me)2)-T-7MeW-K(Ac)-Pen(3)-TMAPF-2Nal-THP- |
| | K(NMeac)-N-3Pya-NMeK(PEG2PEG2K(PEG2PEG2PEG2PEG2gEC12)2)- |
| | CONH2 |
| 410 | cPEG3aCO-Pen(3)-N(N(Me)2)-T-7MeW-K(PEG2PEG2gEC14)-Pen(3)- |
| | TMAPF-2Nal-THP-K(PEG2PEG2gEC14)-N-3Pya-Sar-CON(Me)2 |
| 411 | cPEG3aCO-Pen(3)-Gab-T-7MeW-K(NMeAc)-Pen(3)-APEG3F-2Nal-THP- |
| | K(NMeAc)-N-3Pya-Sar-CON(Me)2 |
| 414 | cPEG3aCO-Pen(3)-N(N(Me)2)-t-7MeW-Dap(NMeAc)-Pen(3)-TMAPF-2Nal- |
| | THP-K(NMeAc)-N-3Pya-Sar-CON(Me)2 |
| 415 | cPEG3aCO-Pen(3)-N(N(Me)2)-T-7MeW-K(Ac)-Pen(3)-TMAPF-2Nal-THP- |
| | Dap(NMeAc)-N-3Pya-Sar-CON(Me)2 |
| 416 | cPEG3aCO-Pen(3)-Dab(Me)3-T-7MeW-K(NMeAc)-Pen(3)-APEG3F-2Nal- |
| | THP-K(NMeAc)-N-3Pya-Sar-CON(Me)2 |
| 418 | cPEG3aCO-Pen(3)-N-T-7MeW-K(Ac)-Pen(3)-F(4N3)-2Nal-THP-K(Ac)-N- |
| | 3Pya-Sar-CONH2 |
| 419 | cPEG3aCO-Pen(3)-N(N(Me)2)-T-7MeW-K(NMeAc)-Pen(3)-APEG3F-2Nal- |
| | THP-Dap(NMeAc)-N-3Pya-Sar-CON(Me)2 |
| 420 | cPEG3aCO-Pen(3)-N-T-7MeW-K(Ac)-Pen(3)-F(4TzlTMA3)-2Nal-THP-K(Ac)- |
| | N-3Pya-Sar-CONH2 |
| 421 | cPEG3aCO-Pen(3)-N(N(me)2)-T-7(3NAcPh)W-K(NMeac)-Pen(3)-MMoEF- |
| | 2Nal-THP-K(NMeAc)-N-3Pya-Sar-CON(Me)2 |
| 424 | cPEG3aCO-Pen(3)-N-T-7MeW-K(Ac)-Pen(3)-F(4TzlTMA4)-2Nal-THP-K(Ac)- |
| | N-3Pya-Sar-CONH2 |
| 427 | cPEG3aCO-Pen(3)-N-T-7MeW-K(Ac)-Pen(3)-TMAPF-2Nal-THP-E-N-3Pya- |
| | Sar-CONH2 |
| 428 | cPEG3aCO-Pen(3)-N-T-7MeW-K(Ac)-Pen(3)-TMAPF-2Nal-THP-K(Ac)-N- |
| | 3Pya-Sar-CONH2 |
| 438 | cPEG3aCO-Pen(3)-N-T-7MeW-K(Ac)-Pen(3)-F(4TzlTMA1)-2Nal-THP-K(Ac)- |
| | N-3Pya-Sar-CONH2 |
| 439 | cPEG3aCO-Pen(3)-N-T-7MeW-K(Ac)-Pen(3)-F(4TzlMMo3)-2Nal-THP-K(Ac)- |
| | N-3Pya-Sar-CONH2 |
| 443 | cPEG3aCO-Pen(3)-N-T-7(3NAcPh)W-K(Ac)-Pen(3)-AEF-2Nal-THP-E-N-3Pya- |
| | Sar-CONH2 |
| 444 | cPEG3aCO-Pen(3)-N-T-7(3NAcPh)W-K(Ac)-Pen(3)-TMAPF-2Nal-THP-E-N- |
| | 3Pya-Sar-CONH2 |
| 445 | cPEG3aCO-Pen(3)-N-T-7(3NAcPh)W-K(Ac)-Pen(3)-AEF-2Nal-THP-K(Ac)-N- |
| | 3Pya-Sar-CONH2 |
| 463 | cPEG3aCO-Pen(3)-L-T-7MeW-L-Pen(3)-APEG3f-2Nal-L-K(NMeAc)-N-3Pya- |
| | Sar-CON(Me)2 |
| 464 | cPEG3aCO-Pen(3)-F-T-7MeW-F-Pen(3)-APEG3f-2Nal-F-K(NMeAc)-N-3Pya- |
| | Sar-CON(Me)2 |
| 465 | cPEG3aCO-Pen(3)-W-T-7MeW-W-Pen(3)-APEG3f-2Nal-W-K(NMeAc)-N- |
| | 3Pya-Sar-CON(Me)2 |
| 466 | cPEG3aCO-Pen(3)-N(N(Me)2)-T-7MeW-K(NMeAc)-Pen(3)-APEG3F-2Nal- |
| | THP-A-N-3Pya-Sar-CON(Me)2 |
| 467 | cPEG3aCO-Pen(3)-N(N(Me)2)-T-7MeW-K(NMeAc)-Pen(3)-APEG3F-2Nal- |
| | THP-L-N-3Pya-Sar-CON(Me)2 |
| 468 | cPEG3aCO-Pen(3)-A-T-7MeW-A-Pen(3)-APEG3f-2Nal-A-K(NMeAc)-N-3Pya- |
| | Sar-CON(Me)2 |
| 469 | cPEG3aCO-Pen(3)-N(N(Me)2)-T-7MeW-K(NMeAc)-Pen(3)-APEG3F-2Nal- |
| | THP-F-N-3Pya-Sar-CON(Me)2 |
| 470 | cPEG3aCO-Pen(3)-N(N(Me)2)-T-7MeW-K(NMeAc)-Pen(3)-APEG3F-2Nal- |
| | THP-W-N-3Pya-Sar-CON(Me)2 |
| 471 | cPEG3aCO-Pen(3)-N-T-7MeW-K(Ac)-Pen(3)-AEF(G)-2Nal-THP-K(Ac)-N- |
| | 3Pya-Sar-CONH2 |
| 472 | cPEG3aCO-Pen(3)-N(N(Me)2)-T-7MeW-K(NMeAc)-Pen(3)-Y(OTzlTMA4)- |
| | 2Nal-THP-K(NMeAc)-N-3Pya-Sar-CON(Me)2 |
| 473 | cPEG3aCO-r-Pen(3)-N-T-7MeW-K(Ac)-Pen(3)-AEF-2Nal-THP-E-N- |
| | 5MePyridinAla-Sar-CONH2 |

TABLE 1D
Compounds

| SEQ ID | Harmonized Sequences txt |
|---|---|
| 3 | MeCO-r-Pen(3)-Q-T-W-Q-Pen(3)-AEF-2Nal-THp-aMeE-N-F-NMeDTyr- |
| | CONH2 |
| 4 | MeCO-r-Pen(3)-Q-T-W-Q-Pen(3)-AEF-2Nal-THP-E-N-N-NMe DTyr-CONH2 |
| 5 | MeCO-Pen(3)-N-T-7MeW-K(Ac)-Pen(3)-AEF-2Nal-aMeK-k(Ac)-N-dK- |
| | NMeDTyr-CONH2 |
| 6 | MeCO-Pen(3)-N-T-7MeW-K(Ac)-Pen(3)-AEF-2Nal-aMeK-k(Ac)-N-dL- |
| | NMeDTyr-CONH2 |
| 7 | MeCO-Pen(3)-N-T-7MeW-K(Ac)-Pen(3)-AEF-2Nal-aMeK-K(Ac)-N-F- |
| | NMeDTyr-CONH2 |
| 8 | MeCO-Pen(3)-N-T-7MeW-K(Ac)-Pen(3)-AEF-2Nal-aMeK-K(Ac)-N-H- |
| | NMeDTyr-CONH2 |
| 9 | MeCO-r-Pen(3)-N-T-7MeW-K(Ac)-Pen(3)-AEF-2Nal-aMeK-E-N-H-NMeDTyr- |
| | CONH2 |
| 10 | MeCO-Pen(3)-N-T-7MeW-K(Ac)-Pen(3)-AEF-2Nal-aMeK-K(Ac)-N-Paf- |
| | NMeDTyr-CONH2 |
| 11 | MeCO-r-Pen(3)-N-T-7MeW-K(Ac)-Pen(3)-AEF-2Nal-THP-E-N-h-NMeDTyr- |
| | CONH2 |
| 12 | MeCO-r-Pen(3)-N-T-7MeW-K(Ac)-Pen(3)-AEF-2Nal-THP-E-N-N-NMeDTyr- |
| | CONH2 |
| 13 | MeCO-Pen(3)-N-T-7MeW-K(Ac)-Pen(3)-AEF-2Nal-aMeK-K(Ac)-N-N- |
| | NMeDTyr-CONH2 |
| 14 | MeCO-Pen(3)-N-T-7MeW-K(Ac)-Pen(3)-AEF-2Nal-aMeK-K(Ac)-N-v- |
| | NMeDTyr-CONH2 |
| 15 | MeCO-Pen(3)-N-T-7MeW-K(Ac)-Pen(3)-AEF-2Nal-aMeK-K(Ac)-N-t- |
| | NMeDTyr-CONH2 |
| 16 | MeCO-r-Pen(3)-N-T-7MeW-Cit-Pen(3)-AEF-2Nal-aMeL-E-N-k-NMeDTyr- |
| | CONH2 |
| 17 | MeCO-r-Pen(3)-N-T-7MeW-Cit-Pen(3)-AEF-2Nal-aMeL-E-N-h-NMeDTyr- |
| | CONH2 |
| 18 | MeCO-Pen(3)-N-T-7MeW-K(Ac)-Pen(3)-AEF-2Nal-aMeK-K(Ac)-N-orn- |
| | NMeDTyr-CONH2 |
| 19 | MeCO-Pen(3)-N-T-7MeW-K(Ac)-Pen(3)-AEF-2Nal-aMeK-K(Ac)-N-s- |
| | NMeDTyr-CONH2 |
| 20 | MeCO-Pen(3)-N-T-7MeW-K(Ac)-Pen(3)-AEF-2Nal-aMeK-K(Ac)-N-f- |
| | NMeDTyr-CONH2 |
| 21 | MeCO-Pen(3)-N-T-7MeW-Y-Pen(3)-AEF-2Nal-aMeK-K(Ac)-N-k-NMeDTyr- |
| | CONH2 |
| 22 | MeCO-Pen(3)-N-T-7MeW-4AmPhe-Pen(3)-AEF-2Nal-aMeK-K(Ac)-N-k- |
| | NMeDTyr-CONH2 |
| 23 | MeCO-Pen(3)-N-T-7MeW-Paf(Ac)-Pen(3)-AEF-2Nal-aMeK-K(Ac)-N-k- |
| | NMeDTyr-CONH2 |
| 24 | MeCO-Pen(3)-N-T-7MeW-K(Ac)-Pen(3)-AEF-2Nal-aMeK-K(Ac)-N-y- |
| | NMeDTyr-CONH2 |
| 25 | MeCO-Pen(3)-N-T-7MeW-Cit-Pen(3)-AEF-2Nal-aMeL-K(Ac)-N-k-NMeDTyr- |
| | CONH2 |
| 26 | MeCO-Pen(3)-N-T-7MeW-K(Ac)-Pen(3)-AEF-2Nal-aMeK-K(Ac)-N-h- |
| | NMeDTyr-CONH2 |
| 27 | MeCO-Pen(3)-N-T-7MeW-K(Ac)-Pen(3)-AEF-2Nal-aMeK-K(ac)-N-bAla- |
| | NMeDTyr-CONH2 |
| 28 | MeCO-Pen(3)-N-T-7MeW-K(Ac)-Pen(3)-AEF-2Nal-aMeK-K(ac)-N-bAla- |
| | NMeDTyr-CONH2 |
| 29 | MeCO-Pen(3)-N-T-7MeW-K(Ac)-Pen(3)-AEF-2Nal-aMeK-K(ac)-N-bAla- |
| | NMeDTyr-CONH2 |
| 30 | MeCO-Pen(3)-N-T-7MeW-K(Ac)-Pen(3)-AEF-2Nal-aMeK-K(ac)-N-3Pya- |
| | NMeDTyr-CONH2 |
| 32 | MeCO-Pen(3)-N-T-7MeW-K(Ac)-Pen(3)-AEF-2Nal-aMeK-K(ac)-N-3pya- |
| | NMeDTyr-CONH2 |
| 35 | MeCO-r-Pen(3)-N-T-7MeW-K(Ac)-Pen(3)-AEF-2Nal-THp-E-N-3Pya- |
| | NMeDTyr-CONH2 |

TABLE 1E
Compounds

| SEQ ID | Harmonized Sequences txt |
|---|---|
| 101 | 5cpaCO-Pen(3)-N(N(Me)2)-T-7MeW-Q(N(Me)2)-Pen(3)-TMAPF-2Nal-THP- |
| | Q(N(Me)2)-N-3Pya-Sar-CON(Me)2 |
| 164 | cPEG3aCO-Pen(3)-N(N(Me)2)-T-7MeW-K(NMeAc)-Pen(3)-TMAPF-2Nal- |
| | THP-K(NMeAc)-N-3Pya-Sar-CON(Me)2 |
| 165 | cPEG3aCO-Pen(3)-N(N(Me)2)-T-7MeW-K(NMeAc)-Pen(3)-TMAPF-2Nal- |
| | THP-E-N-3Pya-Sar-CON(Me)2 |
| 186 | MeCO-Pen(3)-n-T-7MeW-K(NMeAc)-Pen(3)-TMAPF-2Nal-THP-K(NMeAc)- |
| | N-3Pya-Sar-CONH2 |
| 188 | cPEG3aCO-Pen(3)-n-T-7MeW-K(NMeAc)-Pen(3)-TMAPF-2Nal-THP- |
| | K(NMeAc)-N-3Pya-Sar-CONH2 |
| 190 | MeCO-Pen(3)-N(N(Me)2)-T-7MeW-K(NMeAc)-Pen(3)-TMAPF-2Nal-THP- |
| | K(NMeAc)-N-3Pya-Sar-CON(Me)2 |
| 219 | MeCO-Pen(3)-N(N(Me)2)-T-7MeW-K(NMeAc)-Pen(3)-TMAPF-2Nal-THP- |
| | K(NMeAc)-N-3Pya-Sar-CO(NHPEG3a) |
| 220 | cPEG3aCO-Pen(3)-N(N(Me)2)-T-7MeW-K(NMeAc)-Pen(3)-TMAPF-2Nal- |
| | THP-K(NMeAc)-N-3Pya-Sar-CO(NHPEG3a) |
| 247 | cPEG3aCO-Pen(3)-N(N(Me)2)-T-7MeW-APEG2Ser(S*)-Pen(3)-TMAPF- |
| | 2Nal-THP-K(NMeAc)-N-3Pya-Sar-CON(Me)2 |
| 248 | cPEG3aCO-Pen(3)-N(N(Me)2)-T-7MeW-APEG2Ser(R*)-Pen(3)-TMAPF- |
| | 2Nal-THP-K(NMeAc)-N-3Pya-Sar-CON(Me)2 |
| 275 | MeCO-Pen(3)-N(N(Me)2)-T-7MeW-K(NMeAc)-Pen(3)-TMAPF-2Nal-THP- |
| | APEG2Ser-N-3Pya-Sar-CON(Me)2 |
| 281 | MeCO-Pen(3)-N-T-7MeW-K(Ac)-Pen(3)-TMAPF-2Nal-THP-K(Ac)-N-3Pya- |
| | Sar-CONH2 |
| 294 | cPEG3aCO-Pen(3)-N(N(me)2)-T-7(3NAcPh)W-K(NMeAc)-Pen(3)-TMAPF- |
| | 2Nal-THP-K(NMeAc)-N-3Pya-Sar-CON(Me)2 |
| 299 | cPEG3aCO-Pen(3)-n-T-7MeW-K(NMeAc)-Pen(3)-TMAPF-2Nal-THP- |
| | K(NMeAc)-N-3Pya-Sar-CON(Me)2 |
| 300 | cPEG3aCO-Pen(3)-N(N(Me))-T-7MeW-K(NMeAc)-Pen(3)-TMAPF-2Nal-THP- |
| | K(NMeAc)-N-3Pya-Sar-CON(Me)2 |
| 301 | cPEG3aCO-Pen(3)-N(N(Me)2)-T-7MeW-K(NMeAC)-Pen(3)-TMAPF-2Nal- |
| | THP-Dab(NMeAc)-N-3Pya-Sar-CON(Me)2 |
| 307 | cPEG3aCO-Pen(3)-N(N(Me)2)-T-7MeW-K(NMeAC)-Pen(3)-TMAPF-2Nal- |
| | THP-K(NMeaC)-N-3Pya-NMeK(PEG2PEG2C12)-CONH2 |
| 308 | cPEG3aCO-Pen(3)-N(N(Me)2)-T-7MeW-K(Ac)-Pen(3)-TMAPF-2Nal-THP- |
| | Dab(NMeAc)-N-3Pya-Sar-CON(Me)2 |
| 309 | cPEG3aCO-Pen(3)-n-T-7MeW-K(NMeAC)-Pen(3)-TMAPF-2Nal-THP- |
| | Dab(NMeAc)-N-3Pya-Sar-CON(Me)2 |
| 310 | cPEG3aCO-Pen(3)-N(N(Me)2)-T-7MeW-K(NMeAC)-Pen(3)-TMAPF-2Nal- |
| | THP-Q(N(Me)2)-N-3Pya-Sar-CON(Me)2 |
| 311 | cPEG3aCO-Pen(3)-N-t-7MeW-Dab(NMeAc)-Pen(3)-TMAPF-2Nal-THP- |
| | K(NMeAC)-N-3Pya-Sar-CON(Me)2 |
| 312 | cPEG3aCO-Pen(3)-N(N(Me)2)-T-7MeW-Q(N(Me)2)-Pen(3)-TMAPF-2Nal- |
| | THP-K(NMeAC)-N-3Pya-Sar-CON(Me)2 |
| 313 | cPEG3aCO-Pen(3)-N(N(Me)2)-T-7MeW-K(Ac)-Pen(3)-TMAPF-2Nal-THP- |
| | K(NMeaC)-N-3Pya-NMeK(PEG2PEG2gEC12)-CONH2 |
| 314 | cPEG3aCO-Pen(3)-N(N(Me)2)-T-7MeW-K(NMeAC)-Pen(3)-TMAPF-2Nal- |
| | THP-K(NMeaC)-N-3Pya-NMeK(PEG2PEG2gEC12)-CONH2 |
| 315 | cPEG3aCO-Pen(3)-N(N(Me)2)-T-7MeW-K(NMeAC)-Pen(3)-TMAPF-2Nal- |
| | THP-K(NMeaC)-N-3Pya-NMeK(PEG2PEG2gEC14)-CONH2 |
| 316 | cPEG3aCO-Pen(3)-N(N(Me)2)-T-7MeW-K(NMeAc)-Pen(3)-TMAPF-2Nal- |
| | THP-A-N-3Pya-Sar-CON(Me)2 |
| 317 | cPEG3aCO-Pen(3)-N-T-7MeW-K(Ac)-Pen(3)-TMAPF-2Nal-THP-A-N-3Pya- |
| | Sar-CON(Me)2 |
| 322 | C12gEPEG2PEG2CO-hk(Me)3-Pen(3)-N(N(Me)2)-T-7MeW-K(NMeAc)- |
| | Pen(3)-TMAPF-2Nal-THP-K(NMeAc)-N-3Pya-Sar-CON(Me)2 |
| 323 | C12gEPEG2PEG2CO-hk(Me)3-Pen(3)-N(N(Me)2)-T-7MeW- |
| | K(PEG2PEG2gEC12)-Pen(3)-TMAPF-2Nal-THP-K(NMeAc)-N-3Pya-Sar- |
| | CON(Me)2 |
| 324 | C14gEPEG2PEG2CO-hk(Me)3-Pen(3)-N(N(Me)2)-T-7MeW-K(NMeAc)- |
| | Pen(3)-TMAPF-2Nal-THP-K(NMeAc)-N-3Pya-Sar-CON(Me)2 |
| 325 | C14gEPEG2PEG2CO-hk(Me)3-Pen(3)-N(N(Me)2)-T-7MeW- |
| | K(PEG2PEG2gEC14)-Pen(3)-TMAPF-2Nal-THP-K(NMeAc)-N-3Pya-Sar- |
| | CON(Me)2 |
| 326 | cPEG3aCO-Pen(3)-N(N(Me)2)-T-7MeW-K(NMeAc)-Pen(3)-TMAPF-2Nal- |
| | THP-K(NMeAc)-N-3Pya-Sar-CONH2 |
| 327 | cPEG3aCO-Pen(3)-N(N(Me)2)-T-7MeW-K(NMeAc)-Pen(3)-TMAPF-2Nal- |
| | THP-Cit-N-3Pya-Sar-CON(Me)2 |
| 328 | cPEG3aCO-Pen(3)-K(Me)3-T-7MeW-K(NMeAc)-Pen(3)-TMAPF-2Nal-THP- |
| | K(NMeAc)-N-3Pya-Sar-CON(Me)2 |
| 329 | cPEG3aCO-Pen(3)-N(N(Me)2)-T-7MeW-K(Me)3-Pen(3)-TMAPF-2Nal-THP- |
| | K(NMeAc)-N-3Pya-Sar-CON(Me)2 |
| 330 | cPEG3aCO-Pen(3)-N(N(Me)2)-T-7MeW-K(NMeAc)-Pen(3)-TMAPF-2nal- |
| | Acpx-K(NMeAc)-N-3Pya-Sar-CON(Me)2 |
| 339 | cPEG3aCO-Pen(3)-N(N(Me)2)-T-7MeW-K(NMeAc)-Pen(3)-TMAPF-2nal- |
| | Achx-K(NMeAc)-N-3Pya-Sar-CON(Me)2 |
| 340 | cPEG3aCO-Pen(3)-N(N(Me)2)-T-7MeW-K(NMeAc)-Pen(3)-TMAPF-2Nal- |
| | Chg-K(NMeAc)-N-3Pya-Sar-CON(Me)2 |
| 341 | cPEG3aCO-Pen(3)-N(N(Me)2)-T-7MeW-K(NMeAc)-Pen(3)-TMAPF-2Nal- |
| | Aib-K(NMeAc)-N-3Pya-Sar-CON(Me)2 |
| 342 | cPEG3aCO-Pen(3)-K(Me)3-T-7MeW-K(NMeAc)-Pen(3)-TMAPF-2nal-Achx- |
| | K(NMeAc)-N-3Pya-Sar-CON(Me)2 |
| 343 | 5cpaCO-Pen(3)-N(N(Me)2)-T-7MeW-K(NMeAc)-Pen(3)-TMAPF-2Nal-THP- |
| | K(NMeAc)-N-3Pya-Sar-CON(Me)2 |
| 344 | MeCO-hk(Me)3-Pen(3)-N(N(Me)2)-T-7MeW-K(NMeAc)-Pen(3)-TMAPF- |
| | 2Nal-THP-K(NMeAc)-N-3Pya-Sar-CON(Me)2 |
| 345 | MeCO-r-Pen(3)-N(N(Me)2)-T-7MeW-K(NMeAc)-Pen(3)-TMAPF-2Nal-THP- |
| | K(NMeAc)-N-3Pya-Sar-CON(Me)2 |
| 346 | cPEG3aCO-Pen(3)-N(N(Me)2)-T-7MeW-Cit-Pen(3)-TMAPF-2Nal-THP- |
| | K(NMeAc)-N-3Pya-Sar-CON(Me)2 |
| 347 | cPEG3aCO-k(PEG2PEG2gEC14)-Pen(3)-N(N(Me)2)-T-7MeW-K(Ac)-Pen(3)- |
| | TMAPF-2Nal-THP-K(NMeac)-N-3Pya-NMeK(PEG2PEG2gEC14)-CONH2 |
| 348 | cPEG3aCO-k(PEG2PEG2gEC12)-Pen(3)-N(N(Me)2)-T-7MeW-K(Ac)-Pen(3)- |
| | TMAPF-2Nal-THP-K(NMeac)-N-3Pya-NMeK(PEG2PEG2gEC12)-CONH2 |
| 349 | cPEG3aCO-Pen(3)-N-T-7MeW-K(Ac)-Pen(3)-TMAPF-2Nal-THP- |
| | K(PEG2PEG2gEC12)-N-3Pya-NMeK(PEG2PEG2gEC12)-CONH2 |
| 350 | C14gEPEG2PEG2CO-Pen(3)-N(N(Me)2)-T-7MeW-K(Ac)-Pen(3)-TMAPF- |
| | 2Nal-THP-K(NMeac)-N-3Pya-NMeK(PEG2PEG2gEC14)-CONH2 |
| 351 | cPEG3aCO-Pen(3)-N(N(Me)2)-T-7MeW-K(Ac)-Pen(3)-TMAPF-2Nal-THP- |
| | K(PEG2PEG2gEC14)-N-3Pya-NMeK(PEG2PEG2gEC14)-CONH2 |
| 352 | cPEG3aCO-Pen(3)-N(N(Me)2)-T-7MeW-K(PEG2PEG2gEC12)-Pen(3)- |
| | TMAPF-2Nal-THP-K(NMeac)-N-3Pya-NMeK(PEG2PEG2gEC12)-CONH2 |
| 353 | cPEG3aCO-Pen(3)-N(N(Me)2)-T-7MeW-K(Ac)-Pen(3)-TMAPF-2Nal-THP- |
| | K(PEG2PEG2gEC12)-N-3Pya-NMeK(PEG2PEG2gEC12)-CONH2 |
| 354 | C12gEPEG2PEG2CO-Pen(3)-N(N(Me)2)-T-7MeW-K(Ac)-Pen(3)-TMAPF- |
| | 2Nal-THP-K(NMeac)-N-3Pya-NMeK(PEG2PEG2gEC12)-CONH2 |
| 355 | cPEG3aCO-Pen(3)-N(N(Me)2)-T-7MeW-K(PEG2PEG2gEC14)-Pen(3)- |
| | TMAPF-2Nal-THP-K(NMeac)-N-3Pya-NMeK(PEG2PEG2gEC14)-CONH2 |
| 356 | cPEG3aCO-Pen(3)-K(PEG2PEG2gEC12)-T-7MeW-K(Ac)-Pen(3)-TMAPF- |
| | 2Nal-THP-K(NMeac)-N-3Pya-NMeK(PEG2PEG2gEC12)-CONH2 |
| 357 | cPEG3aCO-Pen(3)-K(PEG2PEG2gEC14)-T-7MeW-K(Ac)-Pen(3)-TMAPF- |
| | 2Nal-THP-K(NMeac)-N-3Pya-NMeK(PEG2PEG2gEC14)-CONH2 |
| 358 | cPEG3aCO-Pen(3)-N(N(Me)2)-t-7MeW-Dab(NMeAc)-Pen(3)-TMAPF-2Nal- |
| | THP-K(NMeAc)-N-3Pya-Sar-CON(Me)2 |
| 359 | cPEG3aCO-Pen(3)-N(N(Me)2)-T-7MeW-K(Ac)-Pen(3)-TMAPF-2Nal-THP- |
| | K(NMeac)-N-3Pya-NMeK(PEG2PEG2C14)-CONH2 |
| 360 | cPEG3aCO-Pen(3)-N(N(Me)2)-T-7MeW-K(Ac)-Pen(3)-TMAPF-2Nal-THP- |
| | K(NMeac)-N-3Pya-NMeK(PEG2PEG6gEC12)-CONH2 |
| 361 | cPEG3aCO-Pen(3)-N(N(Me)2)-T-7MeW-K(Ac)-Pen(3)-TMAPF-2Nal-THP- |
| | K(NMeac)-N-3Pya-NMeK(PEG2PEG6gEC14)-CONH2 |
| 362 | cPEG3aCO-Pen(3)-N(N(Me)2)-t-7MeW-Dab(NMeAc)-Pen(3)-TMAPF-2Nal- |
| | THP-K(NMeAc)-N-3Pya-Sar-CONH2 |
| 370 | cPEG3aCO-k(PEG2PEG6gEC12)-Pen(3)-N(N(Me)2)-T-7MeW-K(Ac)-Pen(3)- |
| | TMAPF-2Nal-THP-K(NMeac)-N-3Pya-NMeK(PEG2PEG6gEC12)-CONH2 |
| 371 | cPEG3aCO-Pen(3)-N-T-7MeW-K(Ac)-Pen(3)-TMAPF-2Nal-THP-K(NMeac)- |
| | N-3Pya-NMeK(PEG2PEG2gEC12)-CONH2 |
| 372 | cPEG3aCO-Pen(3)-N(N(Me)2)-T-7MeW-K(Ac)-Pen(3)-TMAPF-2Nal-THP- |
| | K(NMeac)-N-3Pya-NMeK(PEG2PEG2K(PEG2PEG2gEC12)2)-CONH2 |
| 373 | cPEG3aCO-Pen(3)-N(N(Me)2)-T-7MeW-K(Ac)-Pen(3)-TMAPF-2Nal-THP- |
| | K(PEG2PEG2PEG2gEC12))-N-3Pya-NMeK(PEG2PEG2PEG2gEC12)-CONH2 |
| 374 | C14gEPEG2PEG2CO-hk(Me)3-Pen(3)-K(PEG2PEG2gEC14)-T-7MeW- |
| | K(NMeAc)-Pen(3)-TMAPF-2Nal-THP-K(NMeAc)-N-3Pya-Sar-CON(Me)2 |
| 375 | C12gEPEG2PEG2CO-hk(Me)3-Pen(3)-N(N(Me)2)-T-7MeW-K(NMeAc)- |
| | Pen(3)-TMAPF-2Nal-THP-K(PEG2PEG2gEC12)-N-3Pya-Sar-CON(Me)2 |
| 376 | C14gEPEG2PEG2CO-hk(Me)3-Pen(3)-N(N(Me)2)-T-7MeW-K(NMeAc)- |
| | Pen(3)-TMAPF-2Nal-THP-K(PEG2PEG2gEC14)-N-3Pya-Sar-CON(Me)2 |
| 377 | C12gEPEG2PEG2CO-hk(Me)3-Pen(3)-N(N(Me)2)-T-7MeW-K(NMeAc)- |
| | Pen(3)-TMAPF-2Nal-K(PEG2PEG2gEC12)-N-3Pya-Sar-CON(Me)2 |
| 378 | cPEG3aCO-k(PEG2PEG2gEC12)-Pen(3)-N(N(Me)2)-T-7MeW-K(NMeAc)- |
| | Pen(3)-TMAPF-2Nal-THP-K(NMeAc)-N-3Pya-Sar-CON(Me)2 |
| 379 | cPEG3aCO-k(PEG2PEG2gEC14)-Pen(3)-N(N(Me)2)-T-7MeW-K(NMeAc)- |
| | Pen(3)-TMAPF-2Nal-THP-K(NMeAc)-N-3Pya-Sar-CON(Me)2 |
| 380 | cPEG3aCO-k(PEG2PEG2gEC12)-Pen(3)-K(PEG2PEG2gEC12)-T-7MeW- |
| | K(NMeAc)-Pen(3)-TMAPF-2Nal-THP-K(NMeAc)-N-3Pya-Sar-CON(Me)2 |
| 381 | cPEG3aCO-k(PEG2PEG2gEC14)-Pen(3)-K(PEG2PEG2gEC14)-T-7MeW- |
| | K(NMeAc)-Pen(3)-TMAPF-2Nal-THP-K(NMeAc)-N-3Pya-Sar-CON(Me)2 |
| 382 | cPEG3aCO-k(PEG2PEG2gEC12)-Pen(3)-N(N(Me)2)-T-7MeW- |
| | K(PEG2PEG2gEC12)-Pen(3)-TMAPF-2Nal-THP-K(NMeAc)-N-3Pya-Sar- |
| | CON(Me)2 |
| 384 | cPEG3aCO-Pen(3)-N(N(Me)2)-T-7MeW-K(cPEG3a)-Pen(3)-TMAPF-2Nal- |
| | THP-K(NMeAc)-N-3Pya-Sar-CON(Me)2 |
| 385 | C12gEPEG2PEG2CO-k(PEG2PEG2gEC12)-Pen(3)-N(N(Me)2)-t-7MeW- |
| | Dab(NMeAc)-Pen(3)-TMAPF-2Nal-THP-K(NMeAc)-N-3Pya-Sar-CON(Me)2 |
| 386 | C14gEPEG2PEG2CO-k(PEG2PEG2gEC14)-Pen(3)-N(N(Me)2)-t-7MeW- |
| | Dab(NMeAc)-Pen(3)-TMAPF-2Nal-THP-K(NMeAc)-N-3Pya-Sar-CON(Me)2 |
| 387 | cPEG3aCO-Pen(3)-K(PEG2PEG2gEC12)-T-7MeW-K(NMeAc)-Pen(3)-TMAPF- |
| | 2Nal-THP-K(NMeAc)-N-3Pya-Sar-CON(Me)2 |
| 388 | cPEG3aCO-Pen(3)-K(PEG2PEG2gEC14)-T-7MeW-K(NMeAc)-Pen(3)-TMAPF- |
| | 2Nal-THP-K(NMeAc)-N-3Pya-Sar-CON(Me)2 |
| 389 | cPEG3aCO-Pen(3)-K(PEG2PEG2gEC12)-T-7MeW-K(PEG2PEG2gEC12)- |
| | Pen(3)-TMAPF-2Nal-THP-K(NMeAc)-N-3Pya-Sar-CON(Me)2 |
| 390 | cPEG2gCO-Pen(3)-N(N(Me)2)-T-7MeW-K(NMeAc)-Pen(3)-TMAPF-2Nal- |
| | THP-K(NMeAc)-N-3Pya-Sar-CON(Me)2 |
| 391 | cPEG3gCO-Pen(3)-N(N(Me)2)-T-7MeW-K(NMeAc)-Pen(3)-TMAPF-2Nal- |
| | THP-K(NMeAc)-N-3Pya-Sar-CON(Me)2 |
| 392 | cPEG3aCO-Pen(3)-N(N(Me)2)-T-7MeW-K(4cpg)-Pen(3)-TMAPF-2Nal-THP- |
| | K(NMeAc)-N-3Pya-Sar-CON(Me)2 |
| 393 | cPEG3aCO-Pen(3)-K(cPEG3a)-T-7MeW-K(NMeAc)-Pen(3)-TMAPF-2Nal- |
| | THP-K(NMeAc)-N-3Pya-Sar-CON(Me)2 |
| 394 | cPEG3aCO-k(PEG2PEG2gEC12)-Pen(3)-N(N(Me)2)-T-7MeW-K(NMeAc)- |
| | Pen(3)-TMAPF-2Nal-THP-K(PEG2PEG2gEC12)-N-3Pya-Sar-CON(Me)2 |
| 395 | cPEG3aCO-k(PEG2PEG2gEC14)-Pen(3)-N(N(Me)2)-T-7MeW-K(NMeAc)- |
| | Pen(3)-TMAPF-2Nal-THP-K(PEG2PEG2gEC14)-N-3Pya-Sar-CON(Me)2 |
| 396 | cPEG3aCO-Pen(3)-K(PEG2PEG2gEC14)-T-7MeW-K(PEG2PEG2gEC14)- |
| | Pen(3)-TMAPF-2Nal-THP-K(NMeAc)-N-3Pya-Sar-CON(Me)2 |
| 397 | cPEG3aCO-Pen(3)-K(PEG2PEG2gEC12)-T-7MeW-K(NMeAc)-Pen(3)-TMAPF- |
| | 2Nal-THP-K(PEG2PEG2gEC12)-N-3Pya-Sar-CON(Me)2 |
| 398 | cPEG3aCO-Pen(3)-K(PEG2PEG2gEC14)-T-7MeW-K(NMeAc)-Pen(3)-TMAPF- |
| | 2Nal-THP-K(PEG2PEG2gEC14)-N-3Pya-Sar-CON(Me)2 |
| 399 | cPEG3aCO-Pen(3)-N(N(Me)2)-T-7MeW-K(PEG2PEG2gEC14)-Pen(3)- |
| | TMAPF-2Nal-THP-K(NMeAc)-N-3Pya-Sar-CON(Me)2 |
| 400 | cPEG3aCO-Pen(3)-N(N(Me)2)-T-7MeW-K(PEG2PEG2gEC12)-Pen(3)- |
| | TMAPF-2Nal-THP-K(PEG2PEG2gEC12)-N-3Pya-Sar-CON(Me)2 |
| 401 | cPEG3aCO-Pen(3)-N(N(Me)2)-T-7MeW-K(NMeAc)-Pen(3)-TMAPF-2Nal- |
| | THP-K(PEG2PEG2gEC12)-N-3Pya-Sar-CON(Me)2 |
| 402 | cPEG3aCO-Pen(3)-N(N(Me)2)-T-7MeW-K(NMeAc)-Pen(3)-TMAPF-2Nal- |
| | THP-K(PEG2PEG2gEC14)-N-3Pya-Sar-CON(Me)2 |
| 403 | 4cpgCO-Pen(3)-N(N(Me)2)-T-7MeW-K(NMeAc)-Pen(3)-TMAPF-2Nal-THP- |
| | K(NMeAc)-N-3Pya-Sar-CON(Me)2 |
| 404 | cPEG3aCO-k(PEG2PEG2PEG2PEG2gEC12)-Pen(3)-N(N(Me)2)-T-7MeW- |
| | K(Ac)-Pen(3)-TMAPF-2Nal-THP-K(NMeac)-N-3Pya- |
| | NMeK(PEG2PEG2PEG2PEG2gEC12)-CONH2 |
| 407 | cPEG3aCO-Pen(3)-N(N(Me)2)-T-7MeW-K(Ac)-Pen(3)-TMAPF-2Nal-THP- |
| | K(NMeac)-N-3Pya-NMeK(SP6PEG2PEG2C12)-CONH2 |
| 408 | cPEG3aCO-Pen(3)-N(N(Me)2)-T-7MeW-K(Ac)-Pen(3)-TMAPF-2Nal-THP- |
| | K(NMeac)-N-3Pya-NMeK(SP6PEG2PEG2gEC12)-CONH2 |
| 409 | cPEG3aCO-Pen(3)-N(N(Me)2)-T-7MeW-K(Ac)-Pen(3)-TMAPF-2Nal-THP- |
| | K(NMeac)-N-3Pya-NMeK(PEG2PEG2K(PEG2PEG2PEG2PEG2gEC12)2)- |
| | CONH2 |
| 410 | cPEG3aCO-Pen(3)-N(N(Me)2)-T-7MeW-K(PEG2PEG2gEC14)-Pen(3)- |
| | TMAPF-2Nal-THP-K(PEG2PEG2gEC14)-N-3Pya-Sar-CON(Me)2 |
| 414 | cPEG3aCO-Pen(3)-N(N(Me)2)-t-7MeW-Dap(NMeAc)-Pen(3)-TMAPF-2Nal- |
| | THP-K(NMeAc)-N-3Pya-Sar-CON(Me)2 |
| 415 | cPEG3aCO-Pen(3)-N(N(Me)2)-T-7MeW-K(Ac)-Pen(3)-TMAPF-2Nal-THP- |
| | Dap(NMeAc)-N-3Pya-Sar-CON(Me)2 |
| 427 | cPEG3aCO-Pen(3)-N-T-7MeW-K(Ac)-Pen(3)-TMAPF-2Nal-THP-E-N-3Pya- |
| | Sar-CONH2 |
| 428 | cPEG3aCO-Pen(3)-N-T-7MeW-K(Ac)-Pen(3)-TMAPF-2Nal-THP-K(Ac)-N- |
| | 3Pya-Sar-CONH2 |
| 430 | MeCO-Pen(3)-N-T-7MeW-K(Ac)-Pen(3)-TMAPF-2Nal-THP-E-N-3Pya-Sar- |
| | CONH2 |
| 444 | cPEG3aCO-Pen(3)-N-T-7(3NAcPh)W-K(Ac)-Pen(3)-TMAPF-2Nal-THP-E-N- |
| | 3Pya-Sar-CONH2 |
| 446 | MeCO-k(Me)3-Pen(3)-N-T-7MeW-K(Ac)-Pen(3)-TMAPF-2Nal-THP-E-N- |
| | 3Pya-Sar-CONH2 |
| 448 | MeCO-k(Me)3-Pen(3)-N-T-7(3NAcPh)W-K(Ac)-Pen(3)-TMAPF-2Nal-THP-E- |
| | N-3Pya-Sar-CONH2 |
| 450 | MeCO-k(Me)3-Pen(3)-N-T-7MeW-K(Ac)-Pen(3)-TMAPF-2Nal-THP-K(Ac)-N- |
| | 3Pya-Sar-CONH2 |
| 451 | MeCO-Pen(3)-N-T-7(3NAcPh)W-K(Ac)-Pen(3)-TMAPF-2Nal-THP-E-N-3Pya- |
| | Sar-CONH2 |
| 452 | MeCO-Pen(3)-N-T-7(3NAcPh)W-K(Ac)-Pen(3)-TMAPF-2Nal-THP-K(Ac)-N- |
| | 3Pya-Sar-CONH2 |
| 462 | MeCO-r-Pen(3)-N-T-7MeW-K(Ac)-Pen(3)-TMAPF-2Nal-THP-E-N-3Pya-Sar- |
| | CONH2 |

TABLE 1F
Compounds

| SEQ | Structure |
|---|---|
| ID NO: | Harmonized Sequences txt |
| 475 | 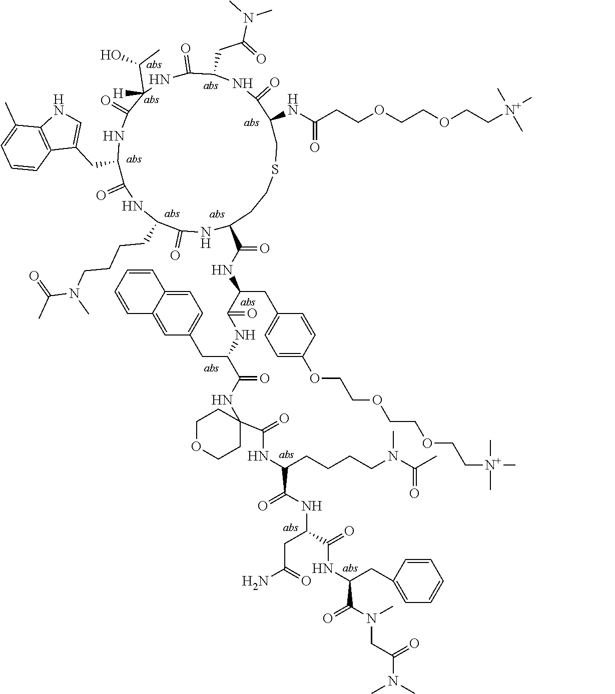 cPEG3aCO-Abu(1)-N(N(Me)2)-T-7MeW-K(NMeAc)-C(1)-APEG3F-2Nal-THP- K(NMeAc)-N-F-Sar-CON(Me)2 |
| 476 | 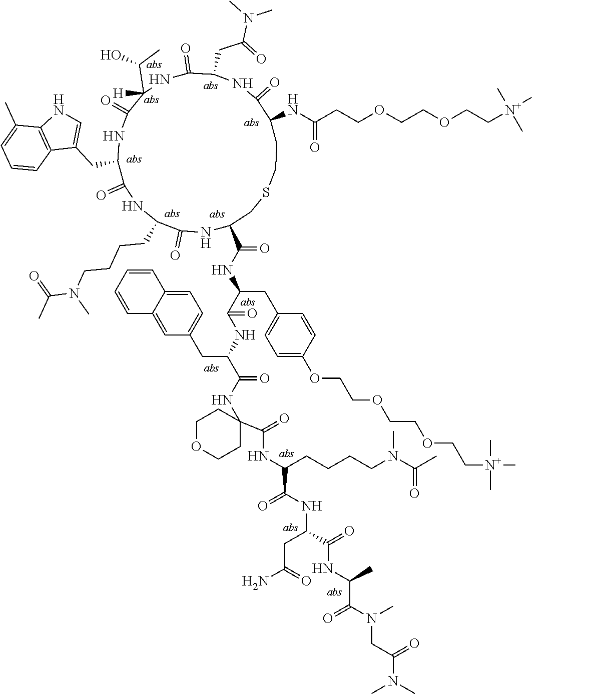 cPEG3aCO-Abu(1)-N(N(Me)2)-T-7MeW-K(NMeAc)-C(1)-APEG3F-2Nal-THP- K(NMeAc)-N-A-Sar-CON(Me)2 |
| 477 | 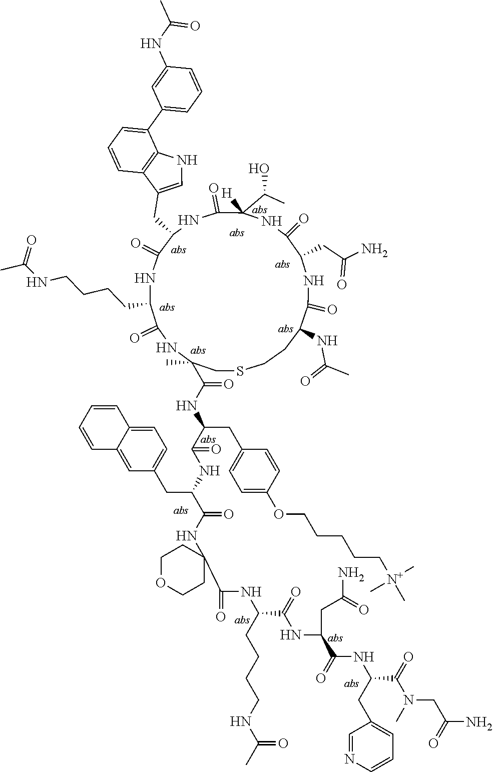 [SMILES: CC(=O)NCCCC[C@@H]1NC(=O)[C@H](Cc2c[nH]c3c(-c4cccc(NC(C)=O)c4)cccc23)NC(=O)[C@H]([C@@H](C)O)NC(=O)[C@H](CC(N)=O)NC(=O)[C@@H](NC(C)=O)CCSC[C@@](C)(C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(N)=O)CCOCC2)NC1=O] MeCO-Abu(1)-N-T-7(3NAcPh)W-K(Ac)-aMeC(1)-TMAPF-2Nal-THP-K(Ac)-N- 3Pya-Sar-CONH2 |
| 478 | 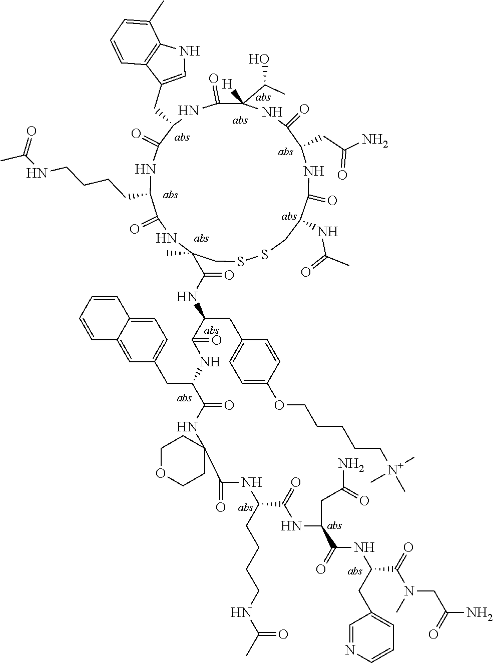 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@H](NC(C)=O)CSSC[C@@](C)(C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(N)=O)CCOCC2)NC1=O] MeCO-C(3)-N-T-7MeW-K(Ac)-aMeC(3)-TMAPF-2Nal-THP-K(Ac)-N-3Pya-Sar- CONH2 |
| 479 | 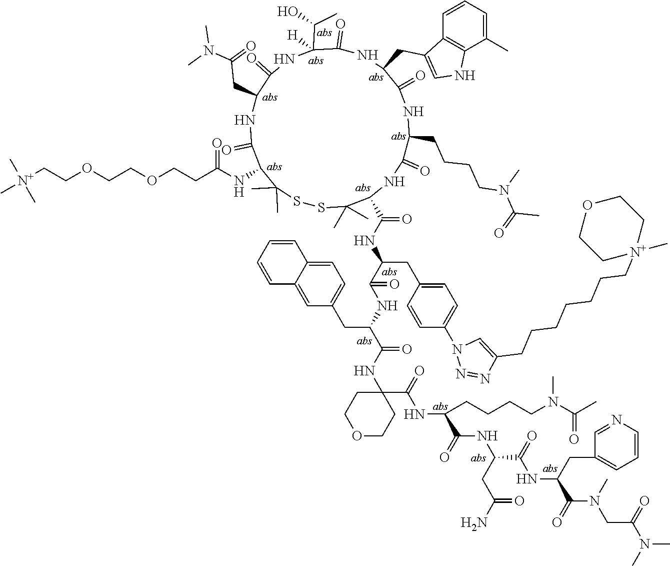 cPEG3aCO-Pen(3)-N(N(Me)2)-T-7MeW-K(NMeAc)-Pen(3)-F(4TzIMMo7)-2Nal- THP-K(NMeAc)-N-3Pya-Sar-CON(Me)2 |
| 480 | 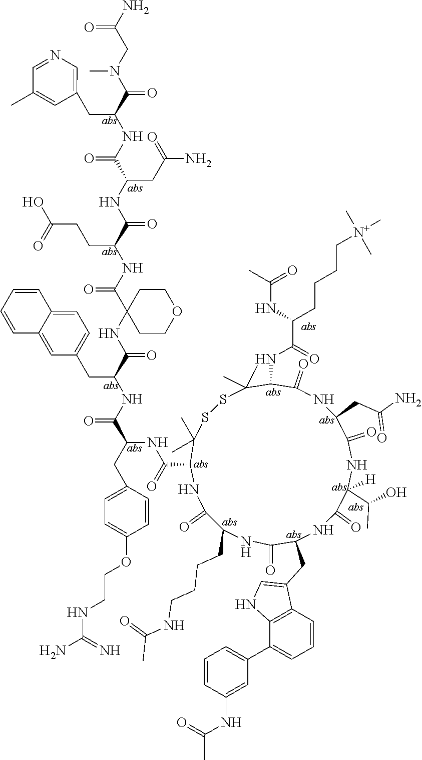 [SMILES: CC(=O)NCCCC[C@@H]1NC(=O)[C@H](Cc2c[nH]c3c(-c4cccc(NC(C)=O)c4)cccc23)NC(=O)[C@H]([C@@H](C)O)NC(=O)[C@H](CC(N)=O)NC(=O)[C@@H](NC(=O)[C@@H](CCCC[N+](C)(C)C)NC(C)=O)C(C)(C)SSC(C)(C)[C@@H](C(=O)N[C@@H](Cc2ccc(OCCNC(=N)N)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cncc(C)c3)C(=O)N(C)CC(N)=O)CCOCC2)NC1=O] MeCO-k(Me)3-Pen(3)-N-T-7(3NAcPh)W-K(Ac)-Pen(3)-AEF(G)-2Nal-THP-E-N- 5MePyridinAla-Sar-CONH2 |
| 481 | 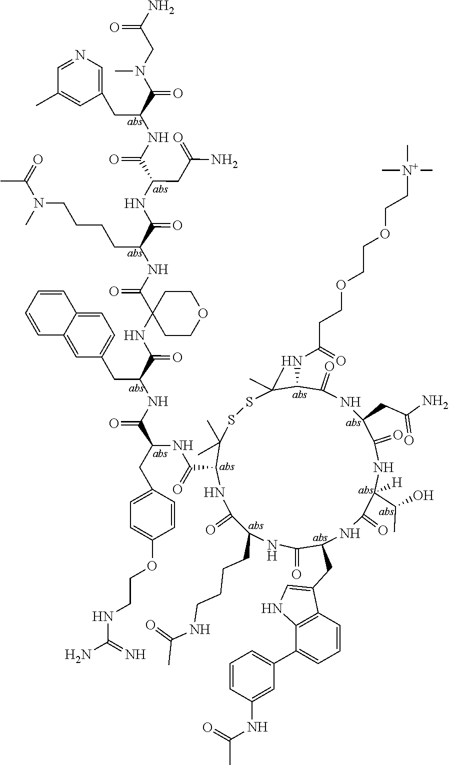 [SMILES: CC(=O)NCCCC[C@@H]1NC(=O)[C@H](Cc2c[nH]c3c(-c4cccc(NC(C)=O)c4)cccc23)NC(=O)[C@H]([C@@H](C)O)NC(=O)[C@H](CC(N)=O)NC(=O)[C@@H](NC(=O)CCOCCOCC[N+](C)(C)C)C(C)(C)SSC(C)(C)[C@@H](C(=O)N[C@@H](Cc2ccc(OCCNC(=N)N)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cncc(C)c3)C(=O)N(C)CC(N)=O)CCOCC2)NC1=O] cPEG3aCO-Pen(3)-N-T-7(3NAcPh)W-K(Ac)-Pen(3)-AEF(G)-2Nal-THP- K(NMeAc)-N-5MePyridinAla-Sar-CONH2 |
| 482 | 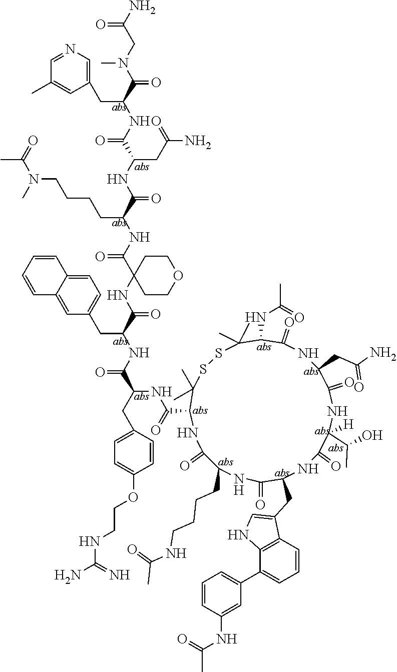 [SMILES: CC(=O)NCCCC[C@@H]1NC(=O)[C@H](Cc2c[nH]c3c(-c4cccc(NC(C)=O)c4)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCNC(=N)N)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cncc(C)c3)C(=O)N(C)CC(N)=O)CCOCC2)NC1=O] MeCO-Pen(3)-N-T-7(3NAcPh)W-K(Ac)-Pen(3)-AEF(G)-2Nal-THP-K(NMeAc)- N-5MePyridinAla-Sar-CONH2 |
| 483 | 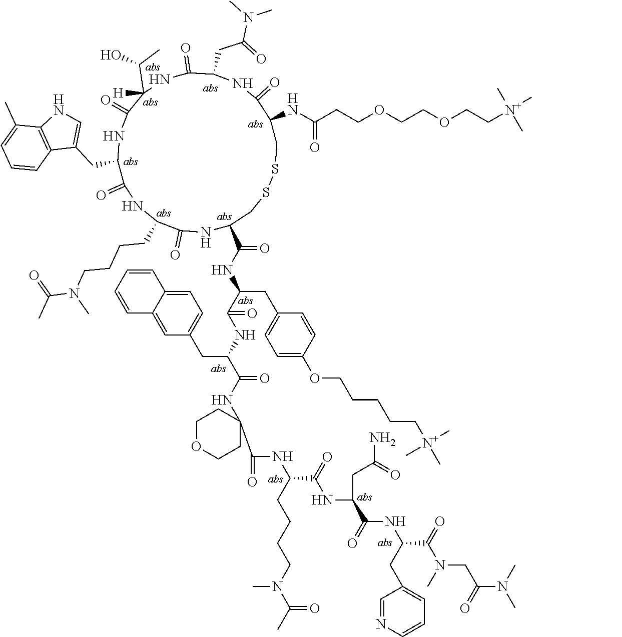 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)CCOCCOCC[N+](C)(C)C)CSSC[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)N(C)C)CCOCC2)NC1=O] cPEG3aCO-C(3)-N(N(Me)2)-T-7MeW-K(NMeAc)-C(3)-TMAPF-2Nal-THP- K(NMeAc)-N-3Pya-Sar-CON(Me)2 |
| 484 | 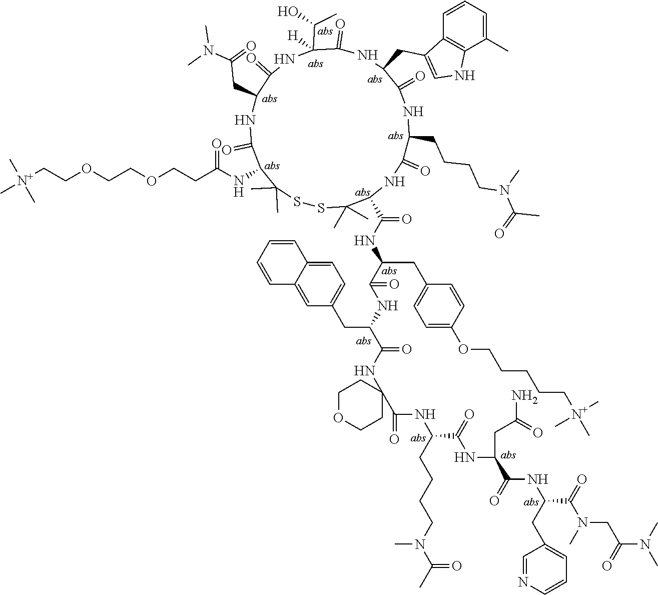 cPEG3aCO-C(3)-N(N(Me)2)-T-7MeW-K(NMeAc)-Pen(3)-TMAPF-2Nal-THP- K(NMeAc)-N-3Pya-Sar-CON(Me)2 |
| 485 | 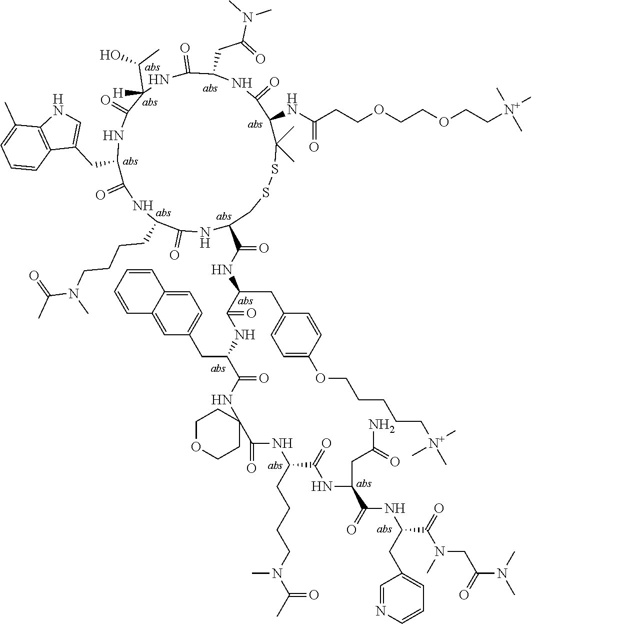 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)CCOCCOCC[N+](C)(C)C)C(C)(C)SSC[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)N(C)C)CCOCC2)NC1=O] cPEG3aCO-Pen(3)-N(N(Me)2)-T-7MeW-K(NMeAc)-C(3)-TMAPF-2Nal-THP- K(NMeAc)-N-3Pya-Sar-CON(Me)2 |
| 486 | 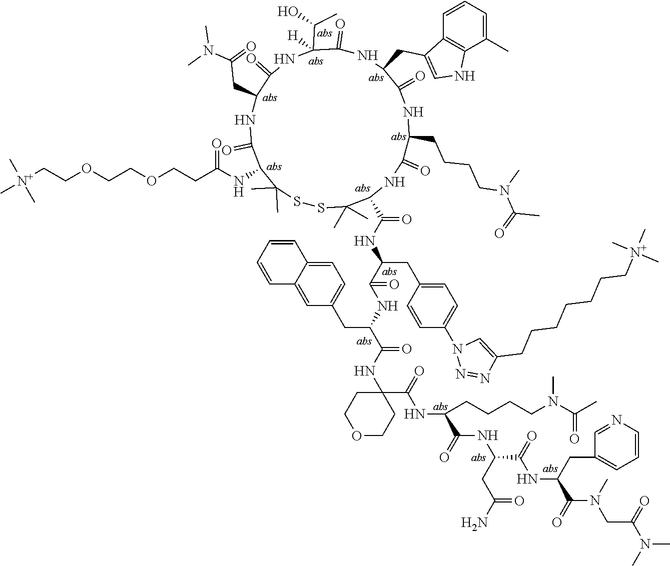 cPEG3aCO-Pen(3)-N(N(Me)2)-T-7MeW-K(NMeAc)-Pen(3)-F(4TzlTMA7)-2Nal- THP-K(NMeAc)-N-3Pya-Sar-CON(Me)2 |
| 487 | 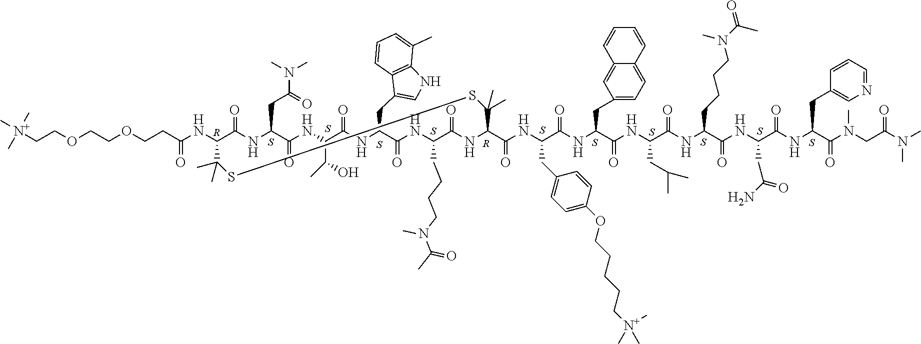 cPEG3aCO-Pen(3)-N(N(Me)2)-T-7MeW-K(NMeAc)-Pen(3)-TMAPf-2Nal-L- K(NMeAc)-N-3Pya-Sar-CON(Me)2 |
| 488 | 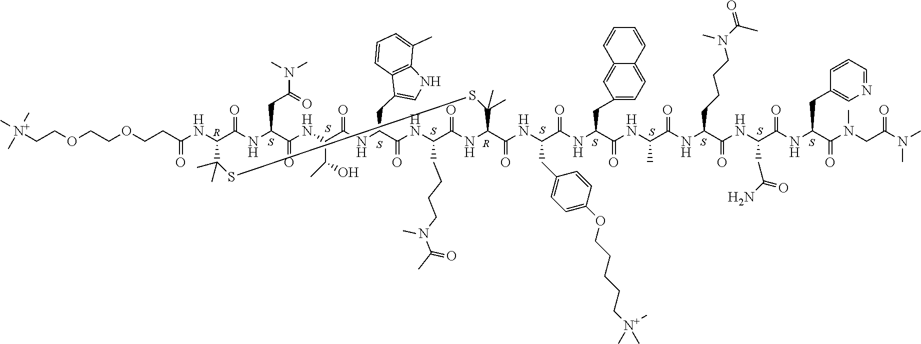 cPEG3aCO-Pen(3)-N(N(Me)2)-T-7MeW-K(NMeAc)-Pen(3)-TMAPf-2Nal-A- K(NMeAc)-N-3Pya-Sar-CON(Me)2 |
| 489 | 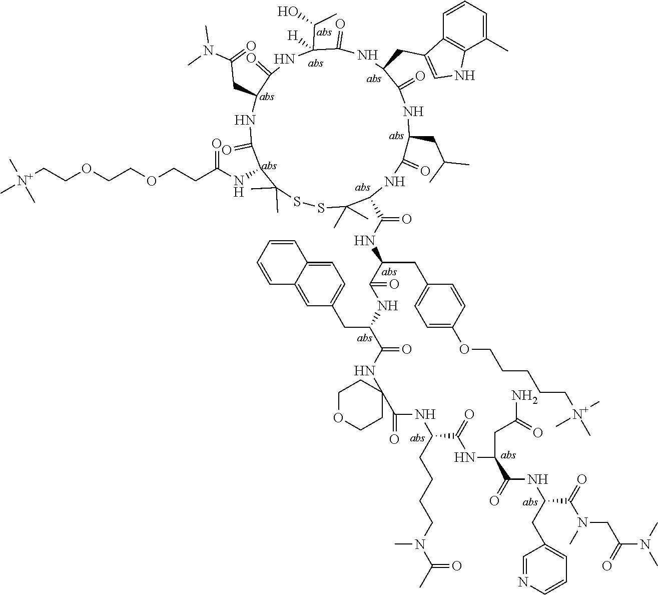 cPEG3aCO-Pen(3)-N(N(Me)2)-T-7MeW-L-Pen(3)-TMAPF-2Nal-THP- K(NMeAc)-N-3Pya-Sar-CON(Me)2 |
| 490 | 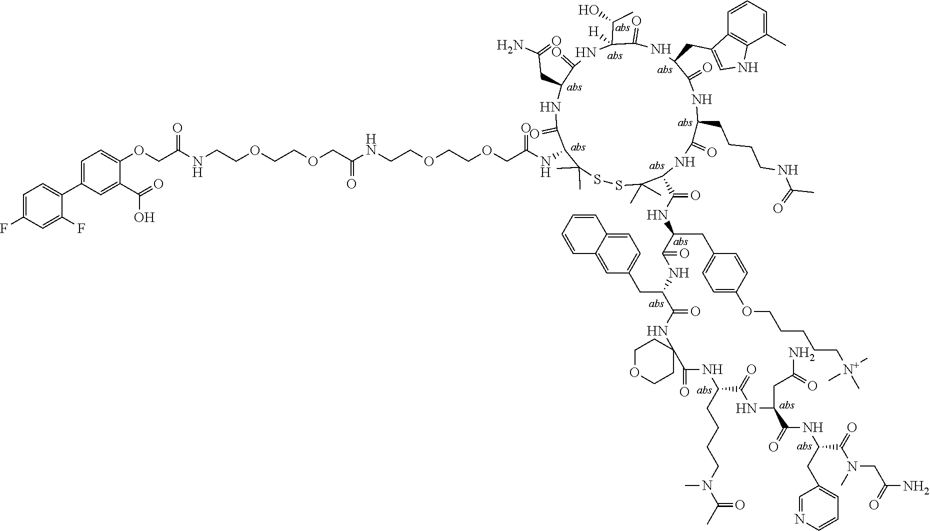 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)COCCOCCNC(=O)COCCOCCNC(=O)COc2ccc(-c3ccc(F)cc3F)cc2C(=O)O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(N)=O)CCOCC2)NC1=O] DFNPEG2PEG2CO-Pen(3)-N-T-7MeW-K(Ac)-Pen(3)-TMAPF-2Nal-THP-K(Ac)- N-3Pya-Sar-CONH2 |
| 491 | 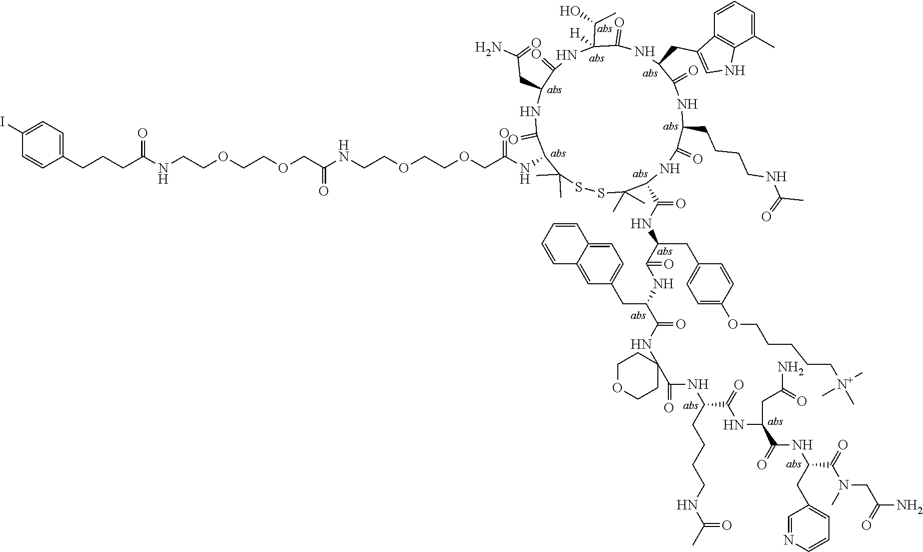 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)COCCOCCNC(=O)COCCOCCNC(=O)CCCc2ccc(I)cc2)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(N)=O)CCOCC2)NC1=O] IPBPEG2PEG2CO-Pen(3)-N-T-7MeW-K(Ac)-Pen(3)-TMAPF-2Nal-THP-K(Ac)- N-3Pya-Sar-CONH2 |
| 492 | 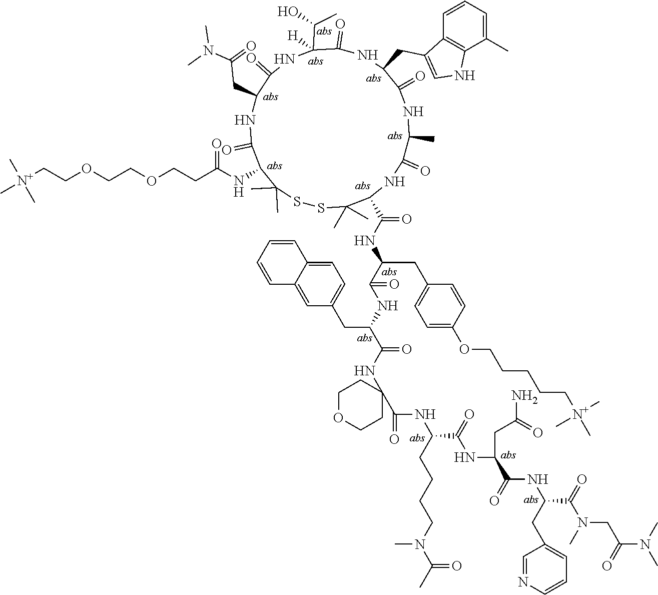 cPEG3aCO-Pen(3)-N(N(Me)2)-T-7MeW-A-Pen(3)-TMAPF-2Nal-THP- K(NMeAc)-N-3Pya-Sar-CON(Me)2 |
| 493 | 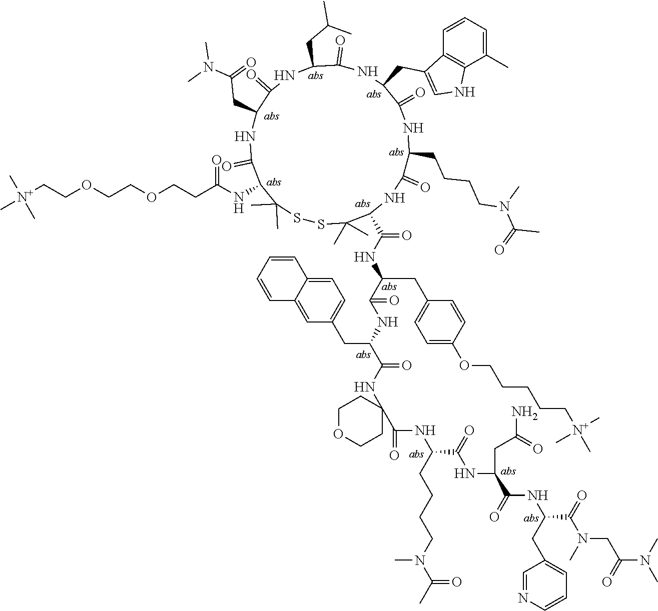 cPEG3aCO-Pen(3)-N(N(Me)2)-L-7MeW-K(NMeAc)-Pen(3)-TMAPF-2Nal-THP- K(NMeAc)-N-3Pya-Sar-CON(Me)2 |
| 494 | 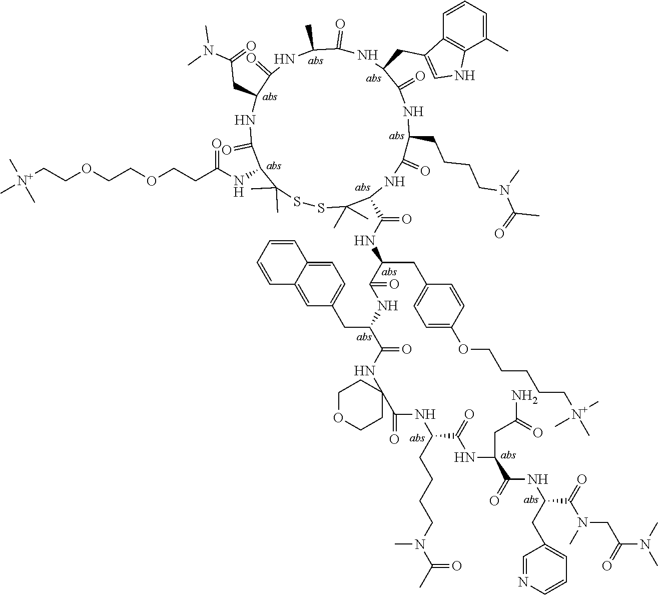 cPEG3aCO-Pen(3)-N(N(Me)2)-A-7MeW-K(NMeAc)-Pen(3)-TMAPF-2Nal-THP- K(NMeAc)-N-3Pya-Sar-CON(Me)2 |
| 495 | 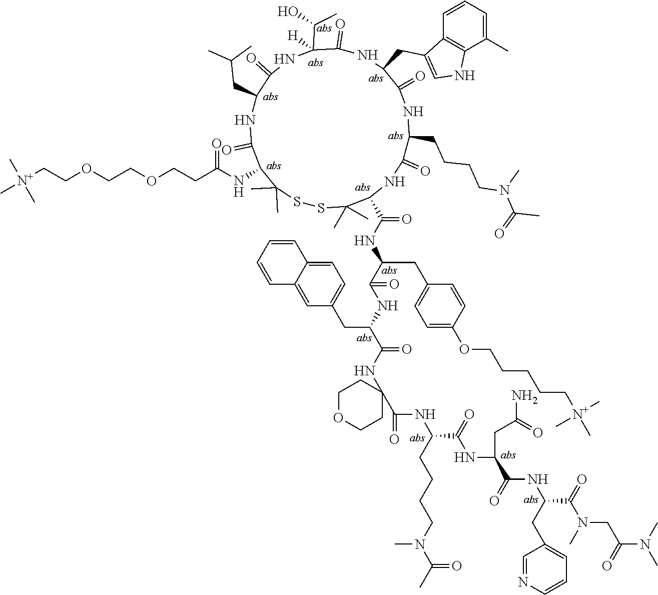 cPEG3aCO-Pen(3)-1-T-7MeW-K(NMeAc)-Pen(3)-TMAPF-2Nal-THP-K(NMeAc)- N-3Pya-Sar-CON(Me)2 |
| 496 | 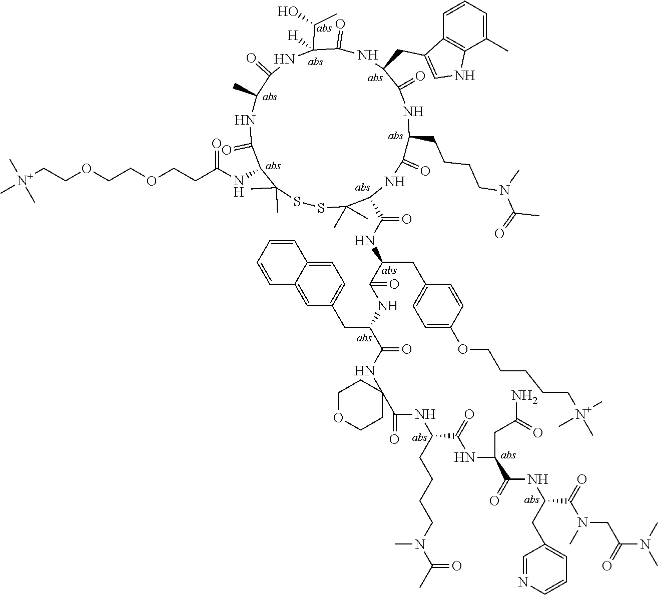 cPEG3aCO-Pen(3)-a-T-7MeW-K(NMeAc)-Pen(3)-TMAPF-2Nal-THP-K(NMeAc)- N-3Pya-Sar-CON(Me)2 |
| 497 | 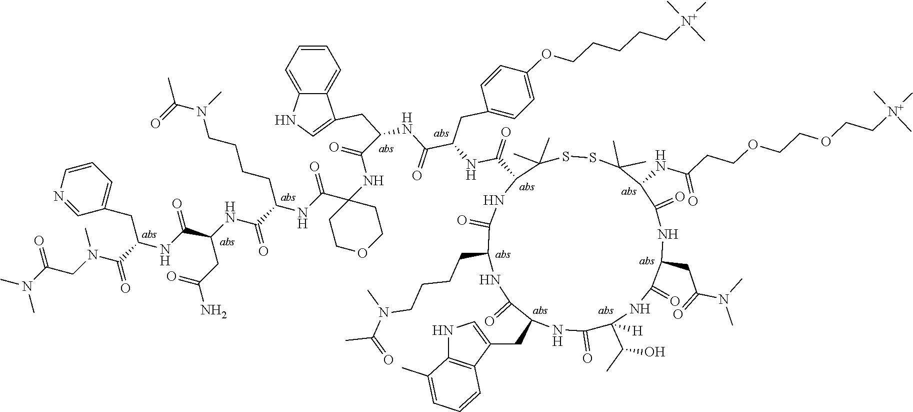 cPEG3aCO-Pen(3)-N(N(Me)2)-T-7MeW-K(NMeAc)-Pen(3)-TMApF-W-THP- K(NMeAc)-N-3Pya-Sar-CON(Me)2 |
| 498 | 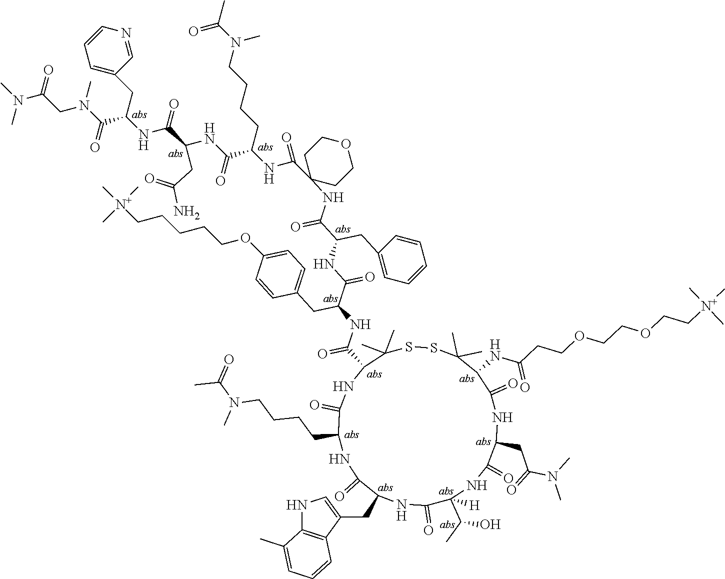 cPEG3aCO-Pen(3)-N(N(Me)2)-T-7MeW-K(NMeAc)-Pen(3)-TMApF-F-THP- K(NMeAc)-N-3Pya-Sar-CON(Me)2 |
| 499 | 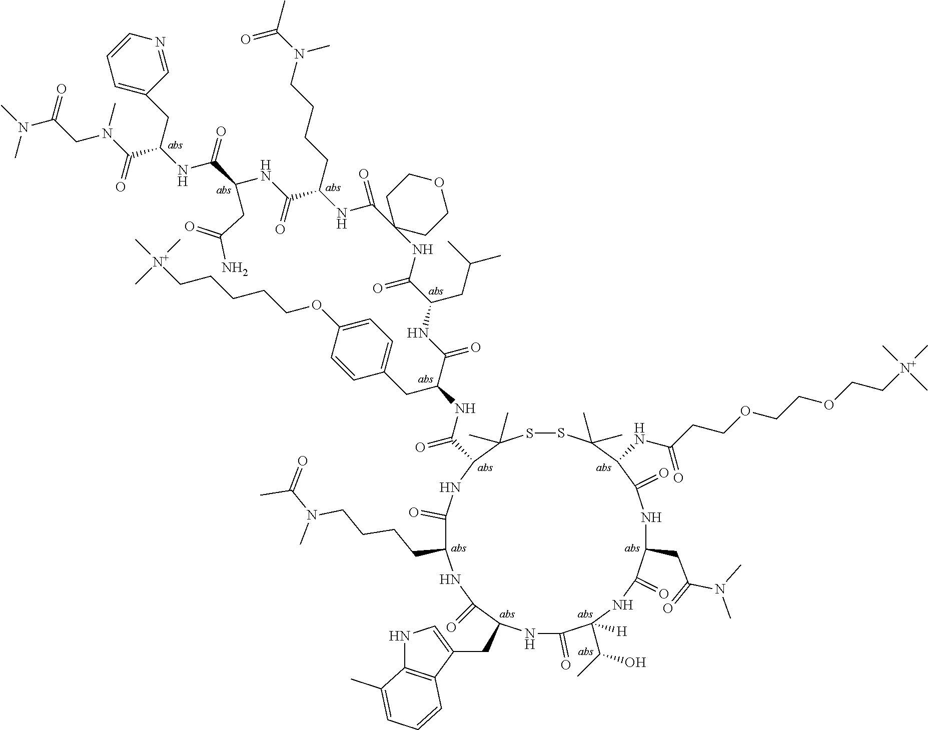 cPEG3aCO-Pen(3)-N(N(Me)2)-T-7MeW-K(NMeAc)-Pen(3)-TMApF-L-THP- K(NMeAc)-N-3Pya-Sar-CON(Me)2 |
| 500 | 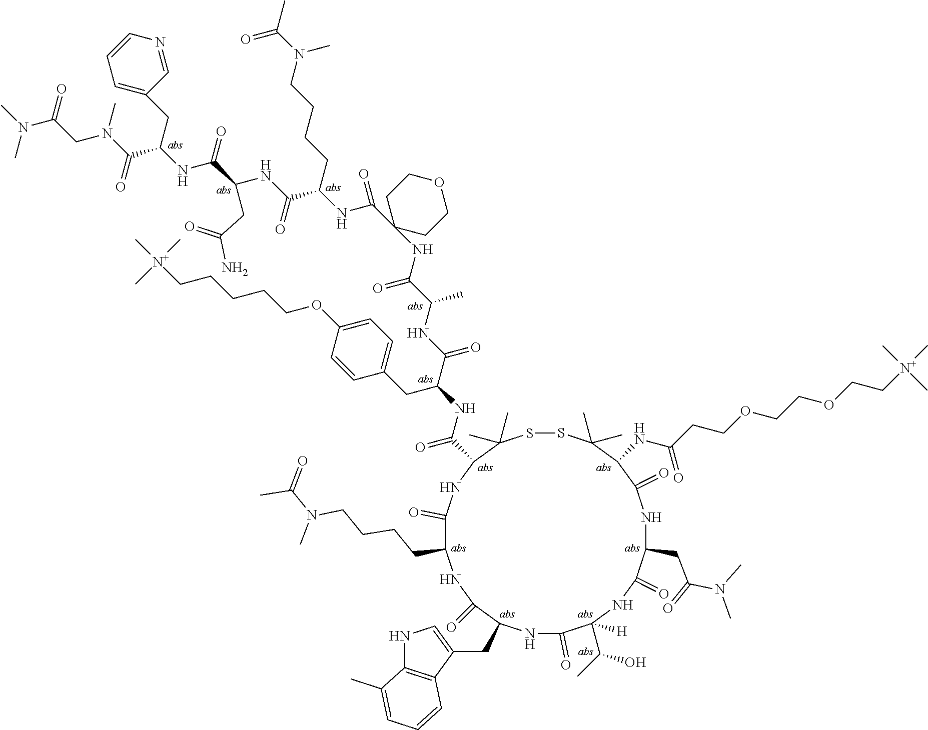 cPEG3aCO-Pen(3)-N(N(Me)2)-T-7MeW-K(NMeAc)-Pen(3)-TMApF-A-THP- K(NMeAc)-N-3Pya-Sar-CON(Me)2 |
| 501 | 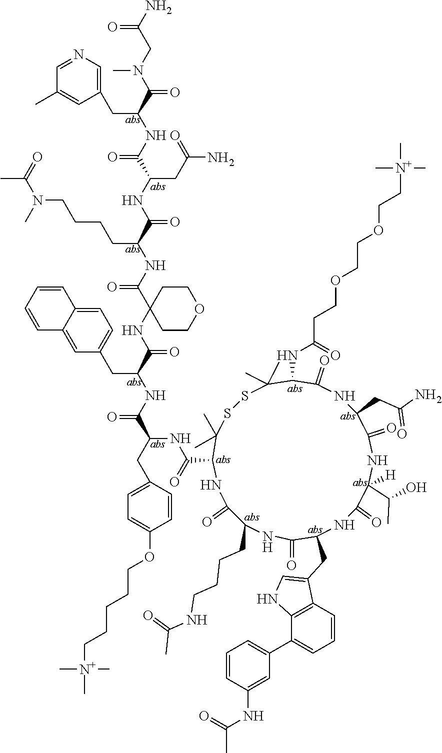 [SMILES: CC(=O)NCCCC[C@@H]1NC(=O)[C@H](Cc2c[nH]c3c(-c4cccc(NC(C)=O)c4)cccc23)NC(=O)[C@H]([C@@H](C)O)NC(=O)[C@H](CC(N)=O)NC(=O)[C@@H](NC(=O)CCOCCOCC[N+](C)(C)C)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cncc(C)c3)C(=O)N(C)CC(N)=O)CCOCC2)NC1=O] cPEG3aCO-Pen(3)-N-T-7(3NAcPh)W-K(Ac)-Pen(3)-TMAPF-2Nal-THP- K(NMeAc)-N-5MePyridinAla-Sar-CONH2 |
| 502 | 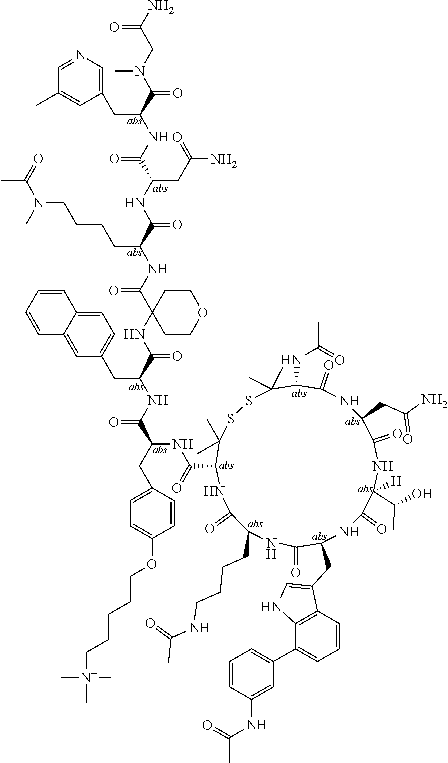 [SMILES: CC(=O)NCCCC[C@@H]1NC(=O)[C@H](Cc2c[nH]c3c(-c4cccc(NC(C)=O)c4)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cncc(C)c3)C(=O)N(C)CC(N)=O)CCOCC2)NC1=O] MeCO-Pen(3)-N-T-7(3NAcPh)W-K(Ac)-Pen(3)-TMAPF-2Nal-THP-K(NMeAc)- N-5MePyridinAla-Sar-CONH2 |
| 503 | 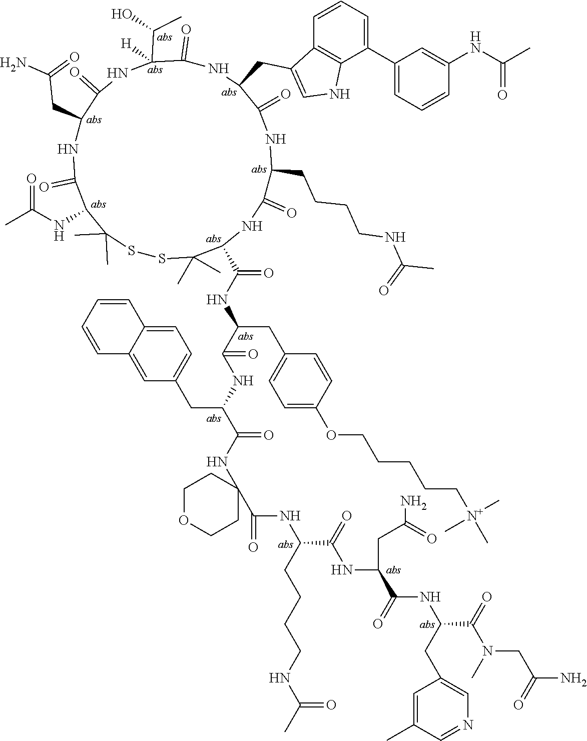 [SMILES: CC(=O)NCCCC[C@@H]1NC(=O)[C@H](Cc2c[nH]c3c(-c4cccc(NC(C)=O)c4)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cncc(C)c3)C(=O)N(C)CC(N)=O)CCOCC2)NC1=O] MeCO-Pen(3)-N-T-7(3NAcPh)W-K(Ac)-Pen(3)-TMAPF-2Nal-THP-K(Ac)-N- 5MePyridinAla-Sar-CONH2 |
| 504 | 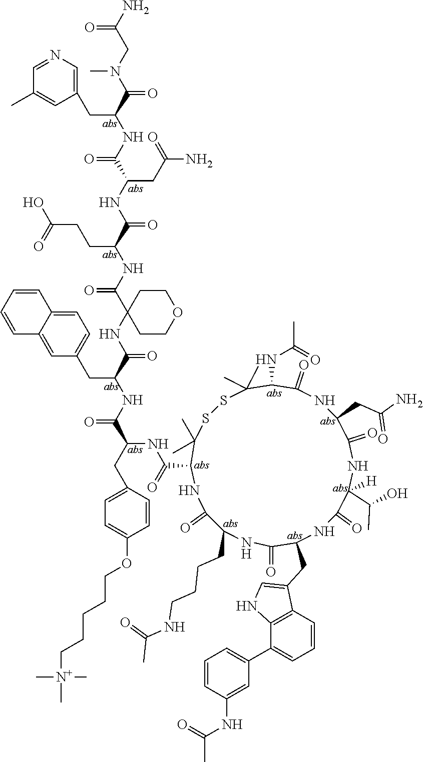 [SMILES: CC(=O)NCCCC[C@@H]1NC(=O)[C@H](Cc2c[nH]c3c(-c4cccc(NC(C)=O)c4)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cncc(C)c3)C(=O)N(C)CC(N)=O)CCOCC2)NC1=O] MeCO-Pen(3)-N-T-7(3NAcPh)W-K(Ac)-Pen(3)-TMAPF-2Nal-THP-E-N- 5MePyridinAla-Sar-CONH2 |
| 505 | 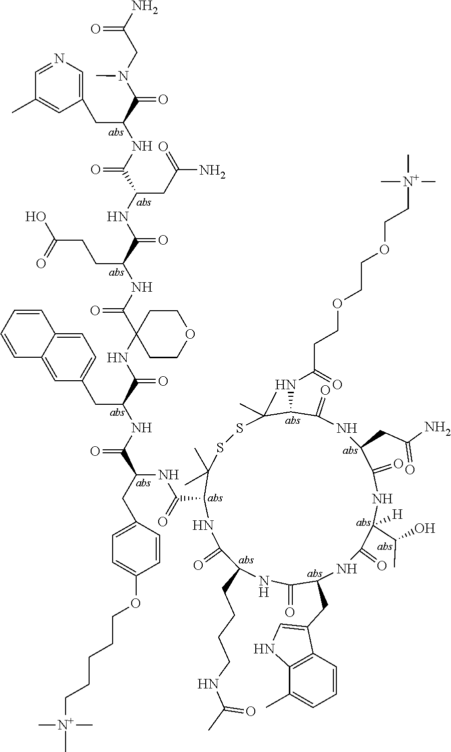 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)CCOCCOCC[N+](C)(C)C)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cncc(C)c3)C(=O)N(C)CC(N)=O)CCOCC2)NC1=O] cPEG3aCO-Pen(3)-N-T-7MeW-K(Ac)-Pen(3)-TMAPF-2Nal-THP-E-N- 5MePyridinAla-Sar-CONH2 |
| 506 | 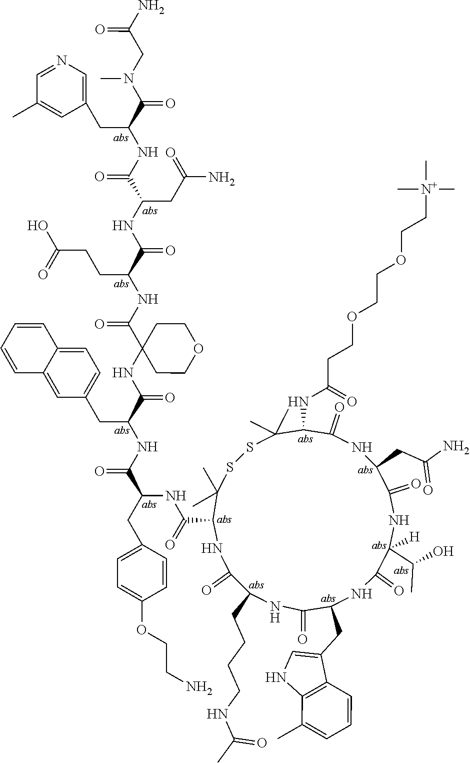 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)CCOCCOCC[N+](C)(C)C)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cncc(C)c3)C(=O)N(C)CC(N)=O)CCOCC2)NC1=O] cPEG3aCO-Pen(3)-N-T-7MeW-K(Ac)-Pen(3)-AEF-2Nal-THP-E-N- 5MePyridinAla-Sar-CONH2 |
| 507 | 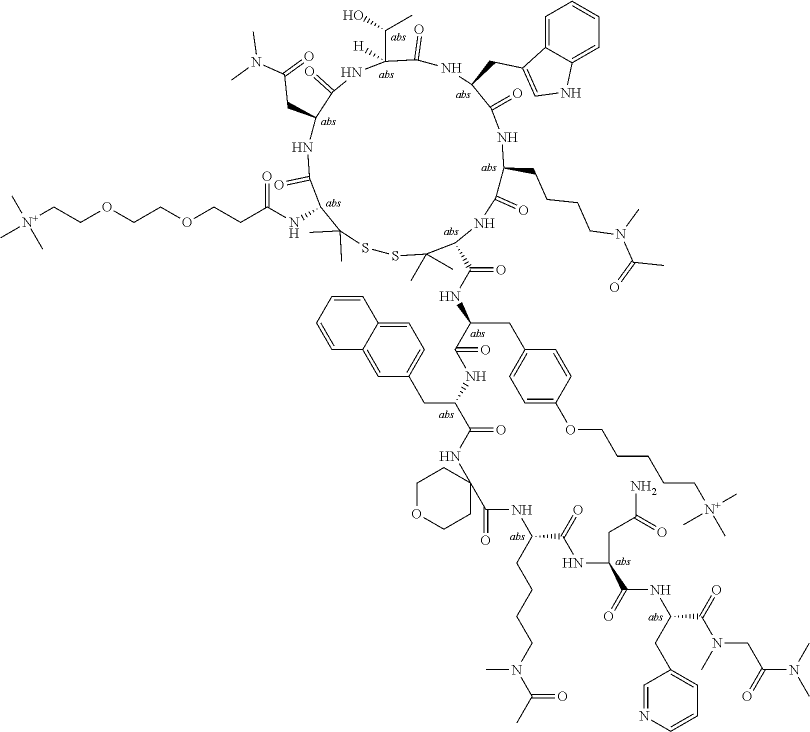 cPEG3aCO-Pen(3)-N(N(Me)2)-T-W-K(NMeAc)-Pen(3)-TMAPF-2Nal-THP- K(NMeAc)-N-3Pya-Sar-CONMe2 |
| 508 | 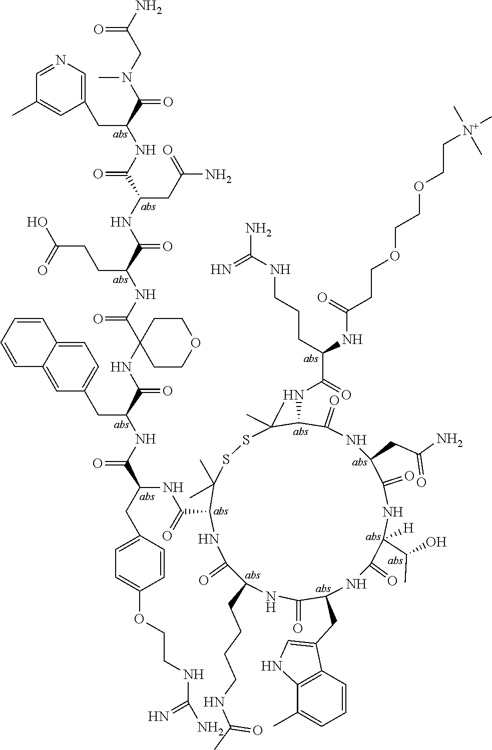 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(=O)CCOCCOCC[N+](C)(C)C)C(C)(C)SSC(C)(C)[C@@H](C(=O)N[C@@H](Cc2ccc(OCCNC(=N)N)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cncc(C)c3)C(=O)N(C)CC(N)=O)CCOCC2)NC1=O] cPEG3aCO-r-Pen(3)-N-T-7MeW-K(Ac)-Pen(3)-AEF(G)-2Nal-THP-E-N- 5MePyridinAla-Sar-CONH2 |
| 509 | 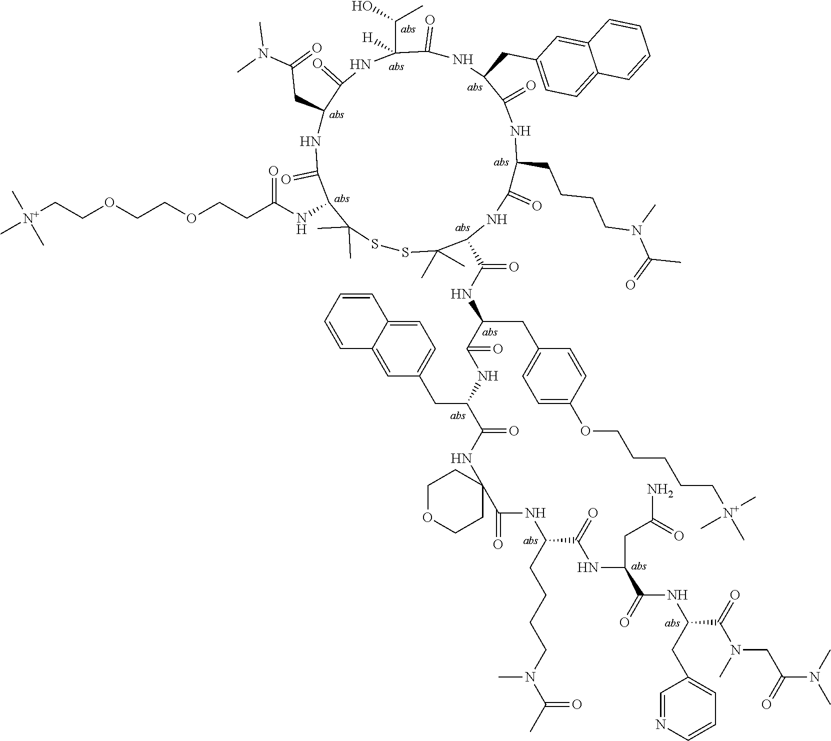 cPEG3aCO-Pen(3)-N(N(Me)2)-T-2Nal-K(NMeAc)-Pen(3)-TMAPF-2Nal-THP- K(NMeAc)-N-3Pya-Sar-CONMe2 |
| 510 | 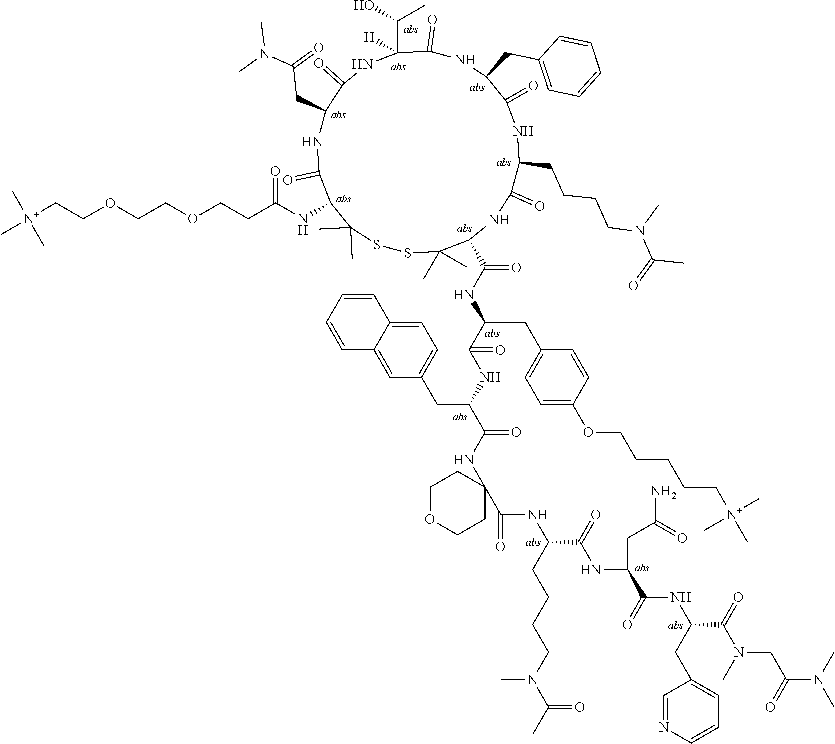 cPEG3aCO-Pen(3)-N(N(Me)2)-T-F-K(NMeAc)-Pen(3)-TMAPF-2Nal-THP- K(NMeAc)-N-3Pya-Sar-CONMe2 |
| 511 | 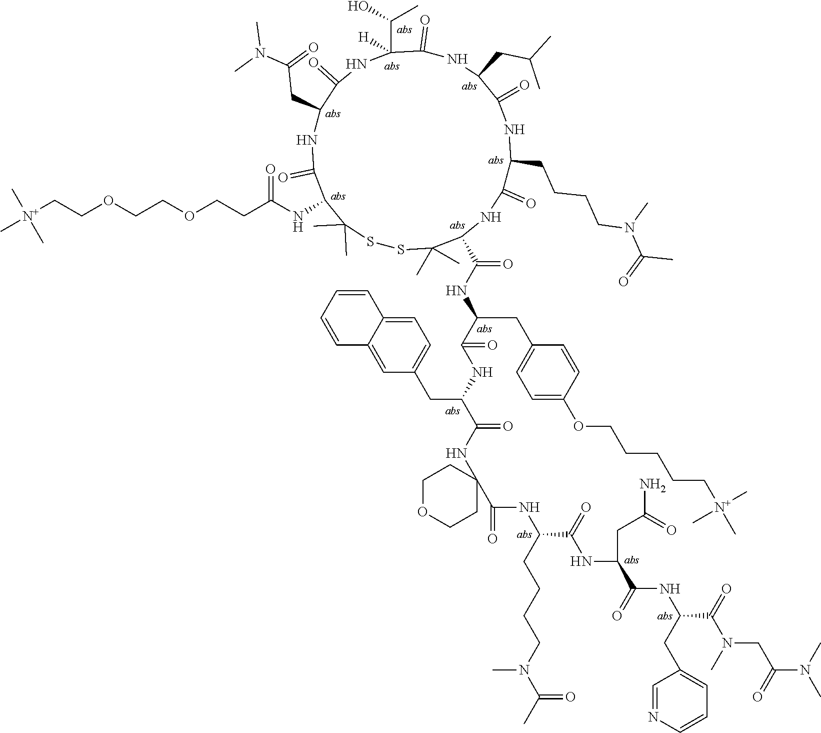 cPEG3aCO-Pen(3)-N(N(Me)2)-T-L-K(NMeAc)-Pen(3)-TMAPF-2Nal-THP- K(NMeAc)-N-3Pya-Sar-CONMe2 |
| 512 | 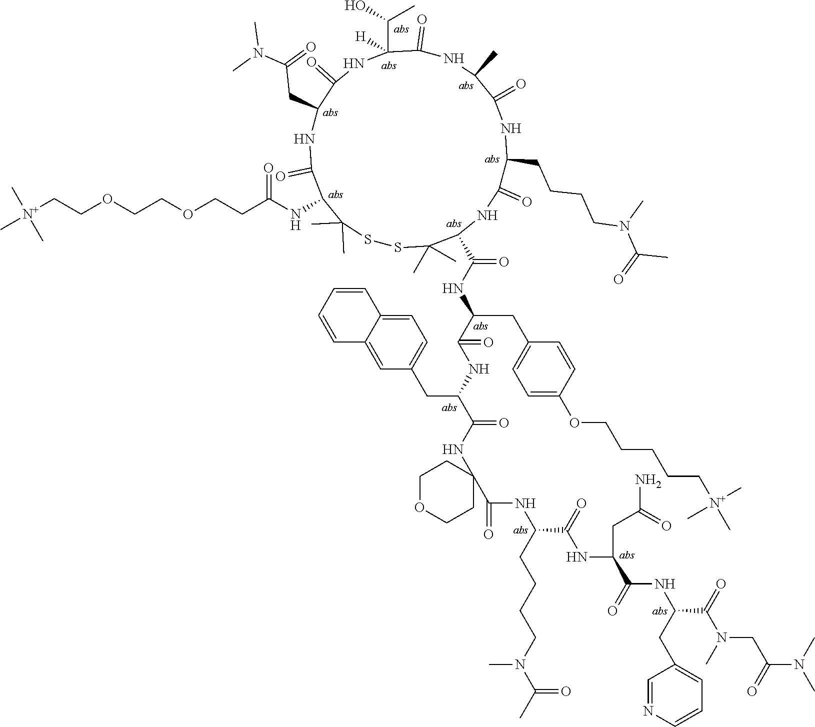 cPEG3aCO-Pen(3)-N(N(Me)2)-T-A-K(NMeAc)-Pen(3)-TMAPF-2Nal-THP- K(NMeAc)-N-3Pya-Sar-CONMe2 |
| 513 | 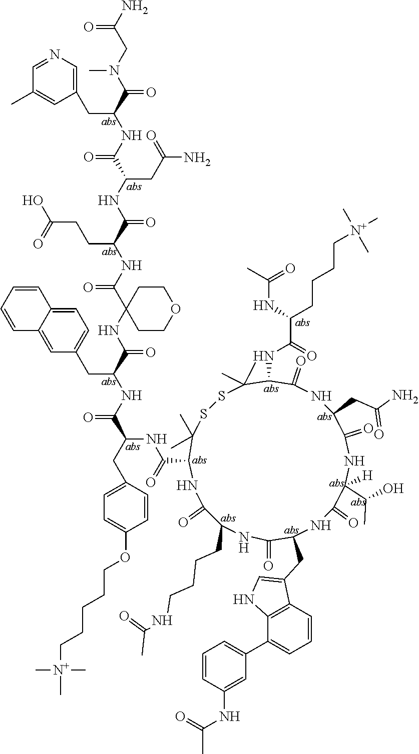 [SMILES: CC(=O)NCCCC[C@@H]1NC(=O)[C@H](Cc2c[nH]c3c(-c4cccc(NC(C)=O)c4)cccc23)NC(=O)[C@H]([C@@H](C)O)NC(=O)[C@H](CC(N)=O)NC(=O)[C@@H](NC(=O)[C@@H](CCCC[N+](C)(C)C)NC(C)=O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cncc(C)c3)C(=O)N(C)CC(N)=O)CCOCC2)NC1=O] MeCO-k(Me)3-Pen(3)-N-T-7(3NAcPh)W-K(Ac)-Pen(3)-TMAPF-2Nal-THP-E-N- 5MePyridinAla-Sar-CONH2 |
| 514 | 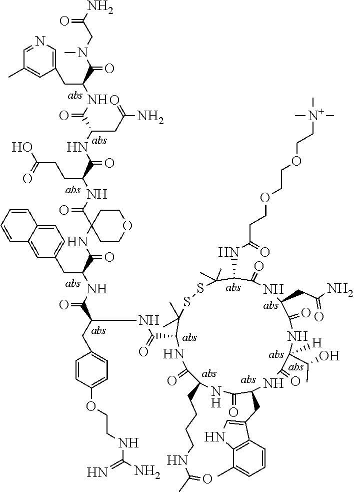 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)CCOCCOCC[N+](C)(C)C)C(C)(C)SSC(C)(C)[C@@H](C(=O)N[C@@H](Cc2ccc(OCCNC(=N)N)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cncc(C)c3)C(=O)N(C)CC(N)=O)CCOCC2)NC1=O] cPEG3aCO-Pen(3)-N-T-7MeW-K(Ac)-Pen(3)-AEF(G)-2Nal-THP-E-N- 5MePyridinAla-Sar-CONH2 |
| 515 | 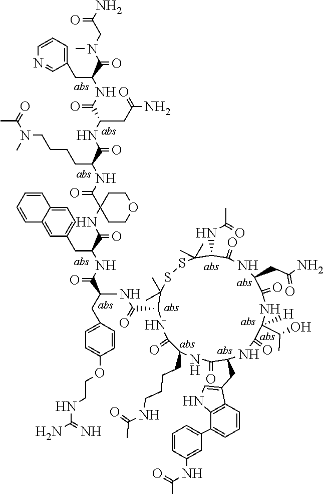 [SMILES: CC(=O)NCCCC[C@@H]1NC(=O)[C@H](Cc2c[nH]c3c(-c4cccc(NC(C)=O)c4)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCNC(=N)N)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(N)=O)CCOCC2)NC1=O] MeCO-Pen(3)-N-T-7(3NAcPh)W-K(Ac)-Pen(3)-AEF(G)-2Nal-THP-K(NMeAc)- N-3Pya-Sar-CONH2 |
| 516 | 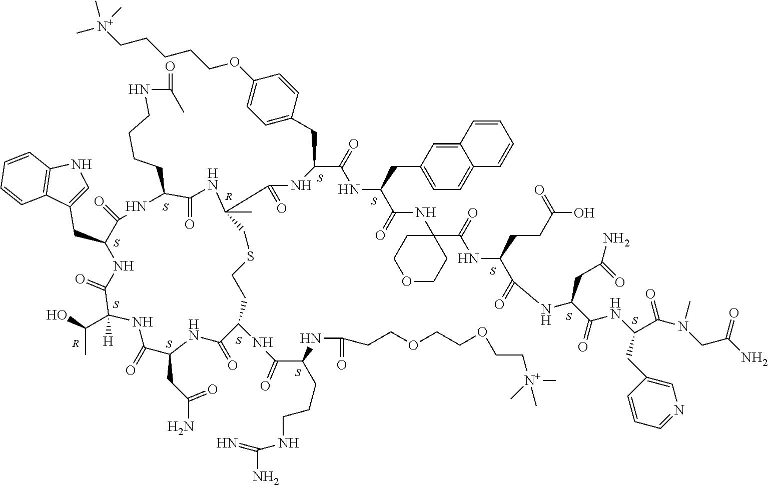 cPEG3aCO-r-Abu(1)-N-T-W-K(Ac)-aMeC(1)-TMAPF-2Nal-THP-E-N-3Pya-Sar- CONH2 |
| 517 | 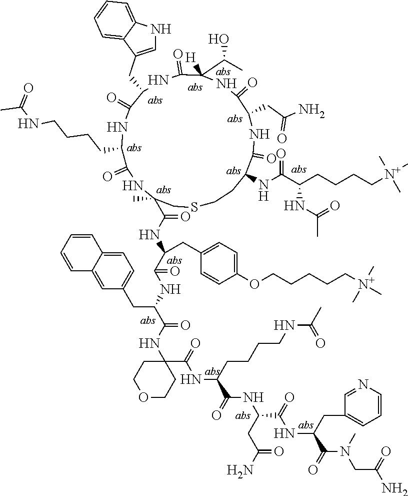 [SMILES: CC(=O)NCCCC[C@@H]1NC(=O)[C@H](Cc2c[nH]c3ccccc23)NC(=O)[C@H]([C@@H](C)O)NC(=O)[C@H](CC(N)=O)NC(=O)[C@@H](NC(=O)[C@H](CCCC[N+](C)(C)C)NC(C)=O)CCSC[C@@](C)(C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(N)=O)CCOCC2)NC1=O] MeCO-k(Me)3-Abu(1)-N-T-W-K(Ac)-aMeC(1)-TMAPF-2Nal-THP-K(Ac)-N- 3Pya-Sar-CONH2 |
| 518 | 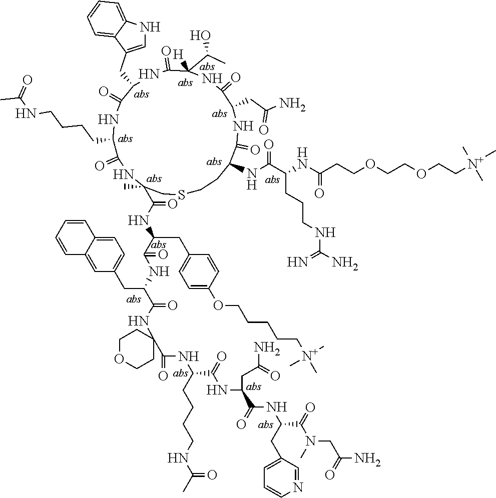 [SMILES: CC(=O)NCCCC[C@@H]1NC(=O)[C@H](Cc2c[nH]c3ccccc23)NC(=O)[C@H]([C@@H](C)O)NC(=O)[C@H](CC(N)=O)NC(=O)[C@@H](NC(=O)[C@@H](CCCNC(=N)N)NC(=O)CCOCCOCC[N+](C)(C)C)CCSC[C@@](C)(C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(N)=O)CCOCC2)NC1=O] cPEG3aCO-r-Abu(1)-N-T-W-K(Ac)-aMeC(1)-TMAPF-2Nal-THP-K(Ac-N-3Pya- Sar-CONH2 |
| 519 | 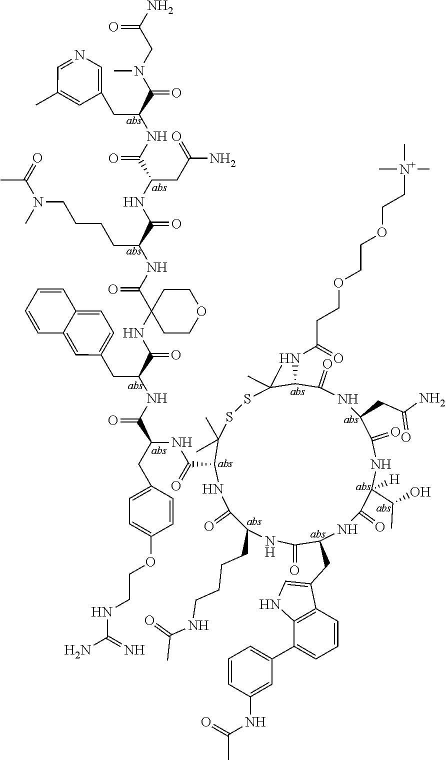 [SMILES: CC(=O)NCCCC[C@@H]1NC(=O)[C@H](Cc2c[nH]c3c(-c4cccc(NC(C)=O)c4)cccc23)NC(=O)[C@H]([C@@H](C)O)NC(=O)[C@H](CC(N)=O)NC(=O)[C@@H](NC(=O)CCOCCOCC[N+](C)(C)C)C(C)(C)SSC(C)(C)[C@@H](C(=O)N[C@@H](Cc2ccc(OCCNC(=N)N)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cncc(C)c3)C(=O)N(C)CC(N)=O)CCOCC2)NC1=O] cPEG3aCO-Pen(3)-N-T-7(3NAcPh)W-K(Ac)-Pen(3)-AEF(G)-2Nal-THP- K(NMeAc)-N-3Pya-Sar-CONH2 |
| 520 | 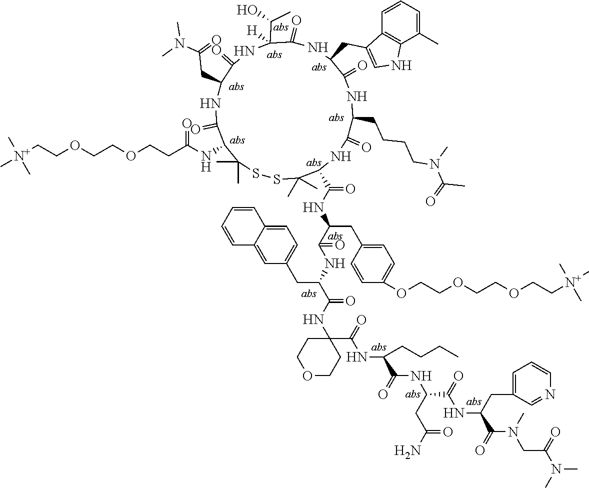 cPEG3aCO-Pen(3)-N(N(Me)2)-T-7MeW-K(NMeAc)-Pen(3)-APEG3F-2Nal-THP- Nle-N-3Pya-Sar-CON(Me)2 |
| 521 | 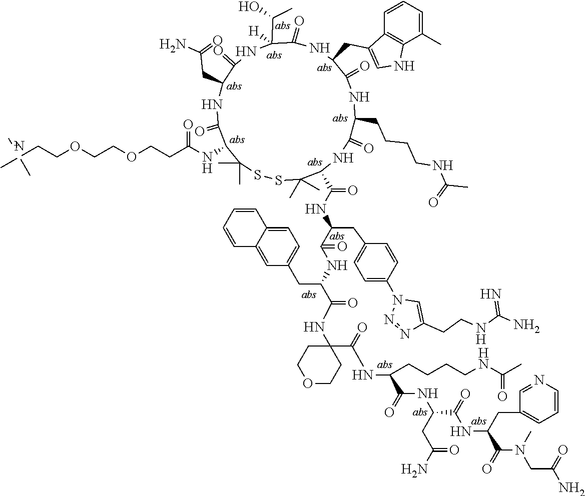 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)CCOCCOCC[N+](C)(C)C)C(C)(C)SSC(C)(C)[C@@H](C(=O)N[C@@H](Cc2ccc(-n3cc(CCNC(=N)N)nn3)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(N)=O)CCOCC2)NC1=O] cPEG3aCO-Pen(3)-N-T-7MeW-K(Ac)-Pen(3)-F(4TzlG2)-2Nal-THP-K(Ac)-N- 3Pya-Sar-CONH2 |
| 522 | 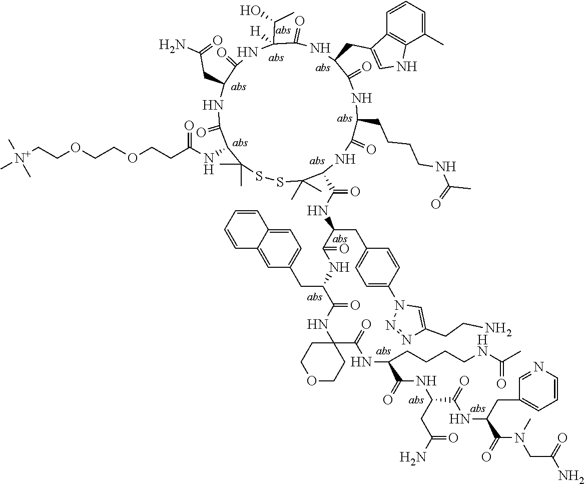 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)CCOCCOCC[N+](C)(C)C)C(C)(C)SSC(C)(C)[C@@H](C(=O)N[C@@H](Cc2ccc(-n3cc(CCN)nn3)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(N)=O)CCOCC2)NC1=O] cPEG3aCO-Pen(3)-N-T-7MeW-K(Ac)-Pen(3)-F(4TzlAme2)-2Nal-THP-K(Ac)-N- 3Pya-Sar-CONH2 |
| 523 | 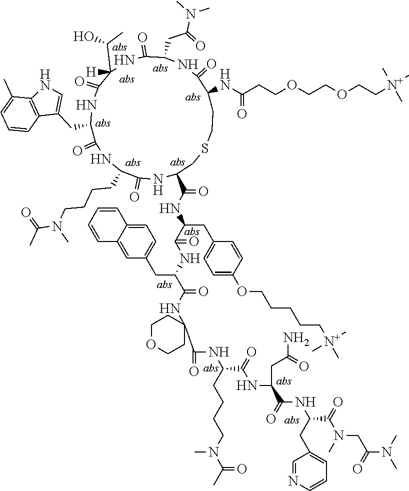 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)CCOCCOCC[N+](C)(C)C)CCSC[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)N(C)C)CCOCC2)NC1=O] cPEG3aCO-Abu(1)-N(N(Me)2)-T-7MeW-K(NMeAc)-C(1)-TMAPF-2Nal-THP- K(NMeAc)-N-3Pya-Sar-CON(Me)2 |
| 524 | 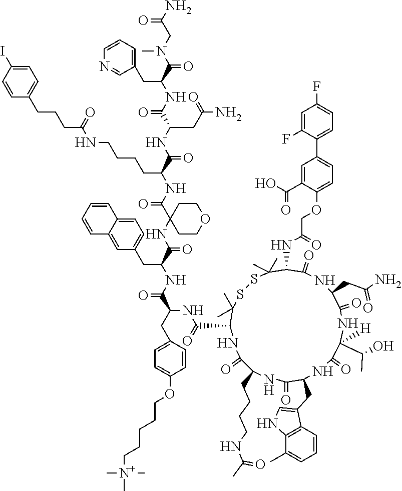 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)COc2ccc(-c3ccc(F)cc3F)cc2C(=O)O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCNC(=O)CCCc3ccc(I)cc3)C(=O)N[C@@H](CC(N)=O)C(=O)N[C@@H](Cc3cccnc3)C(=O)N(C)CC(N)=O)CCOCC2)NC1=O] DFNCO-Pen(3)-N-T-7MeW-K(Ac)-Pen(3)-TMAPF-2Nal-THP-K(IPB)-N-3Pya- Sar-CONH2 |
| 525 | 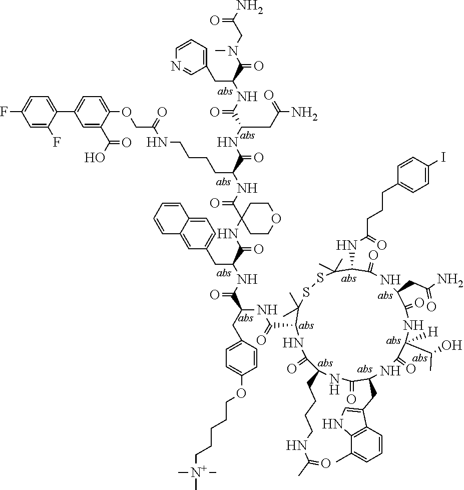 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)CCCc2ccc(I)cc2)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCNC(=O)COc3ccc(-c4ccc(F)cc4F)cc3C(=O)O)C(=O)N[C@@H](CC(N)=O)C(=O)N[C@@H](Cc3cccnc3)C(=O)N(C)CC(N)=O)CCOCC2)NC1=O] IPBCO-Pen(3)-N-T-7MeW-K(Ac)-Pen(3)-TMAPF-2Nal-THP-K(DFN)-N-3Pya- Sar-CONH2 |
| 526 | 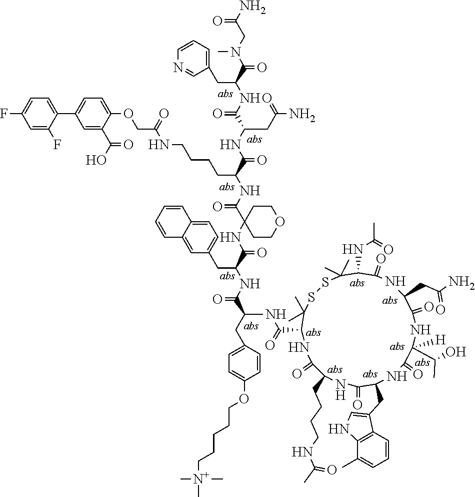 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCNC(=O)COc3ccc(-c4ccc(F)cc4F)cc3C(=O)O)C(=O)N[C@@H](CC(N)=O)C(=O)N[C@@H](Cc3cccnc3)C(=O)N(C)CC(N)=O)CCOCC2)NC1=O] MeCO-Pen(3)-N-T-7MeW-K(Ac)-Pen(3)-TMAPF-2Nal-THP-K(DFN)-N-3Pya- Sar-CONH2 |
| 527 | 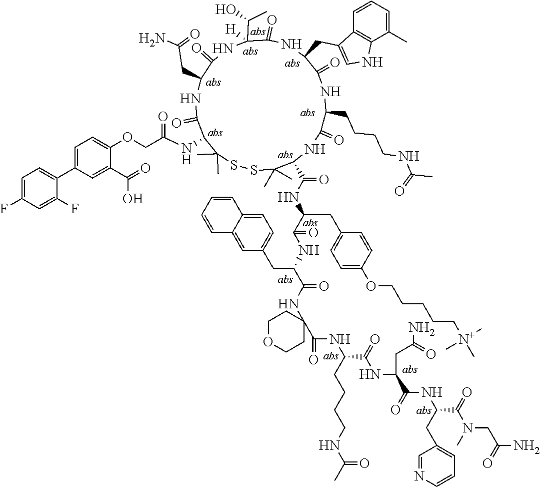 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)COc2ccc(-c3ccc(F)cc3F)cc2C(=O)O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(N)=O)CCOCC2)NC1=O] DFNCO-Pen(3)-N-T-7MeW-K(Ac)-Pen(3)-TMAPF-2Nal-THP-K(Ac)-N-3Pya-Sar- CONH2 |
| 528 | 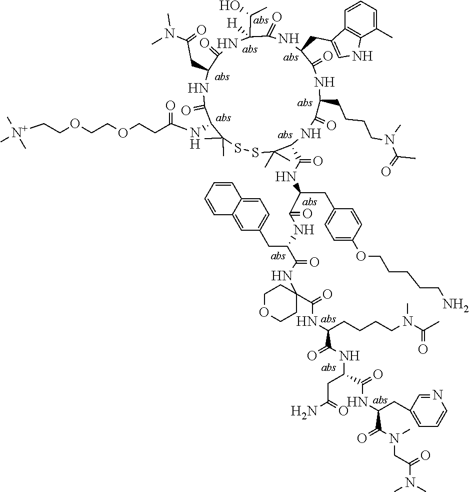 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)CCOCCOCC[N+](C)(C)C)C(C)(C)SSC(C)(C)[C@@H](C(=O)N[C@@H](Cc2ccc(OCCCCCN)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)N(C)C)CCOCC2)NC1=O] cPEG3aCO-Pen(3)-N(N(Me)2)-T-7MeW-K(NMeAc)-Pen(3)-APF-2Nal-THP- K(NMeAc)-N-3Pya-Sar-CON(Me)2 |
| 529 | 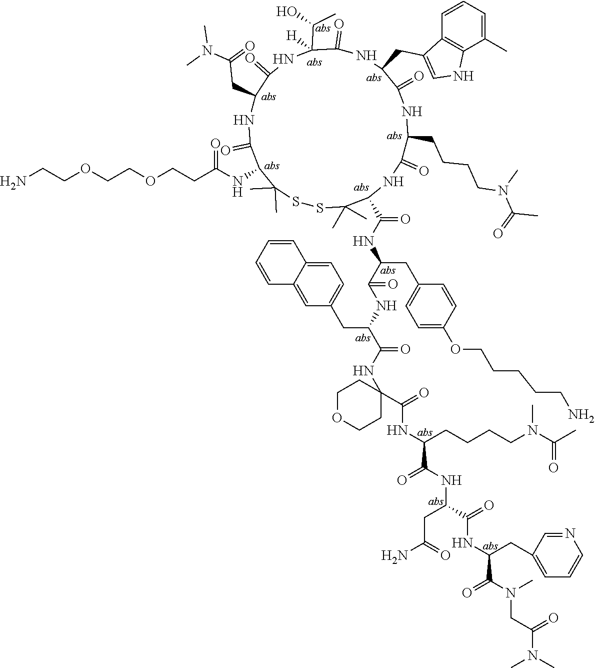 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)CCOCCOCCN)C(C)(C)SSC(C)(C)[C@@H](C(=O)N[C@@H](Cc2ccc(OCCCCCN)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)N(C)C)CCOCC2)NC1=O] cPEG3AmCO-Pen(3)-N(N(Me)2)-T-7MeW-K(NMeAc)-Pen(3)-APF-2Nal-THP- K(NMeAc)-N-3Pya-Sar-CON(Me)2 |
| 530 | 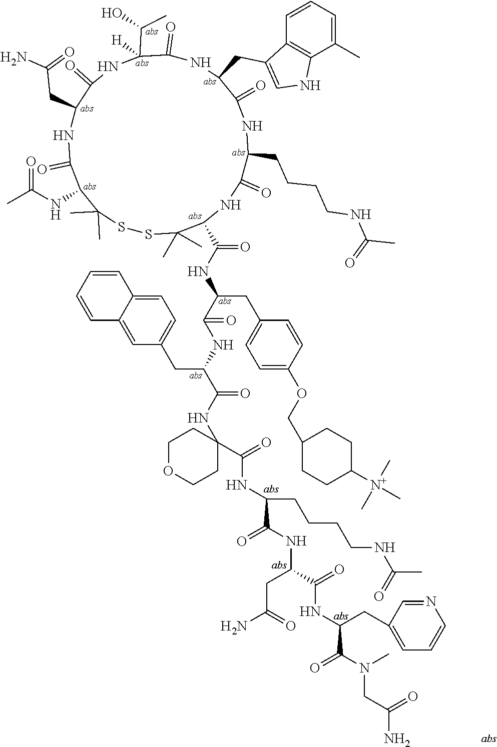 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCC3CCC([N+](C)(C)C)CC3)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(N)=O)CCOCC2)NC1=O] MeCO-Pen(3)-N-T-7MeW-K(Ac)-Pen(3)-ACHMF(S*, S*)-2Nal-THP-K(Ac)-N- 3Pya-Sar-CONH2 |
| 531 | 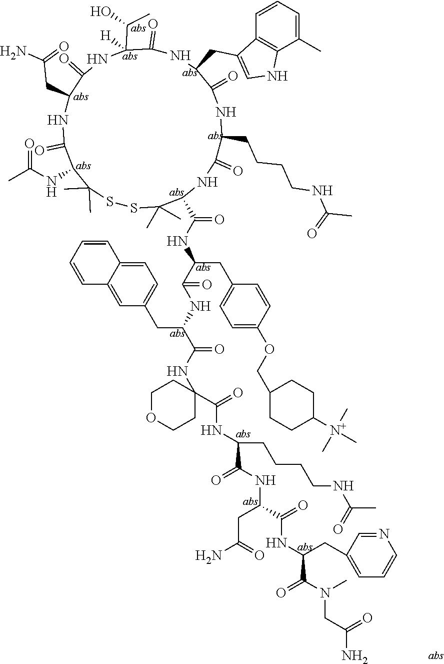 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCC3CCC([N+](C)(C)C)CC3)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(N)=O)CCOCC2)NC1=O] MeCO-Pen(3)-N-T-7MeW-K(Ac)-Pen(3)-ACHMF(R*, S*)-2Nal-THP-K(Ac)-N- 3Pya-Sar-CONH2 |
| 532 | 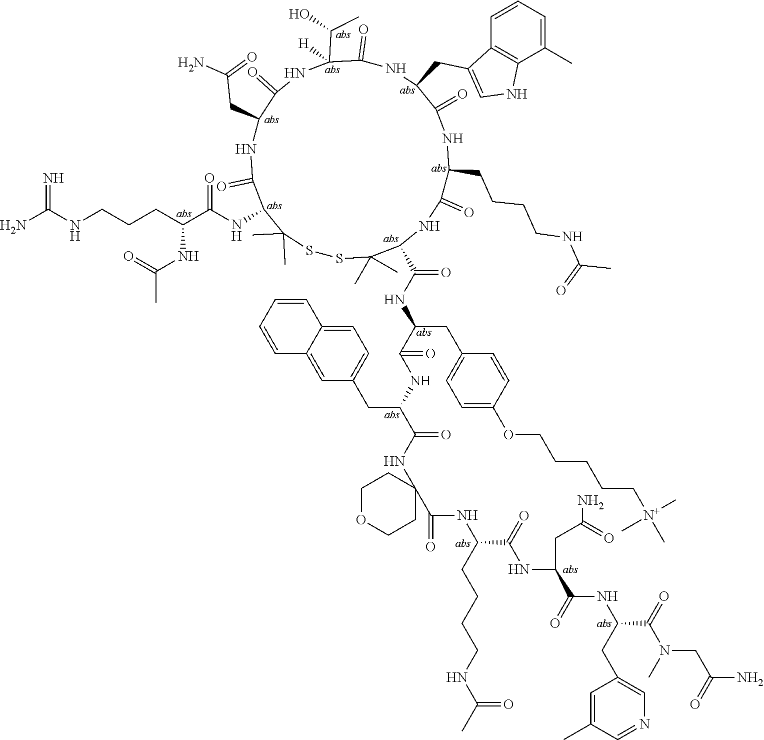 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cncc(C)c3)C(=O)N(C)CC(N)=O)CCOCC2)NC1=O] MeCO-r-Pen(3)-N-T-7MeW-K(Ac)-Pen(3)-TMAPF-2Nal-THP-K(Ac)-N- 5MePyridinAla-Sar-CONH2 |
| 533 | 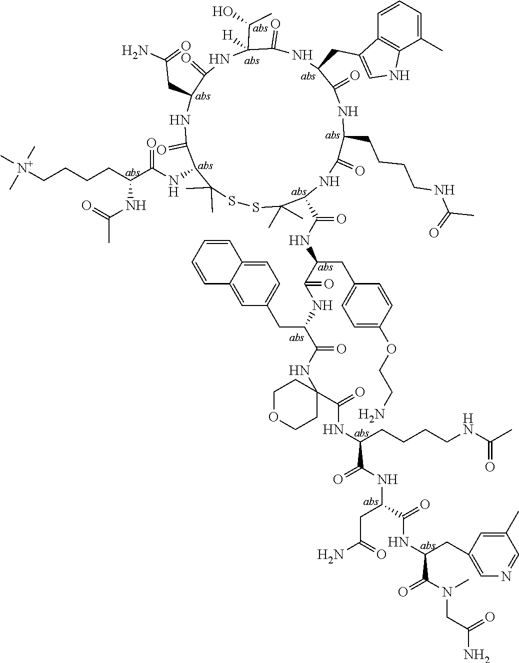 MeCO-k(Me)3-Pen(3)-N-T-7MeW-K(Ac)-Pen(3)-AEF-2Nal-THP-K(Ac)-N- 5MePyridinAla-Sar-CONH2 |
| 534 | 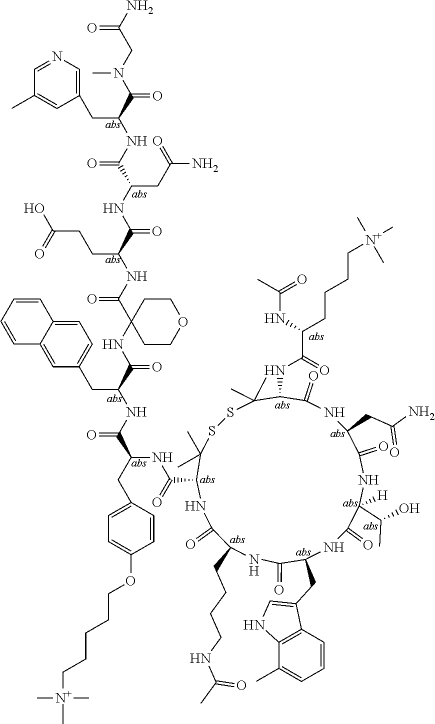 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCC[N+](C)(C)C)NC(C)=O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cncc(C)c3)C(=O)N(C)CC(N)=O)CCOCC2)NC1=O] MeCO-k(Me)3-Pen(3)-N-T-7MeW-K(Ac)-Pen(3)-TMAPF-2Nal-THP-E-N- 5MePyridinAla-Sar-CONH2 |
| 535 | 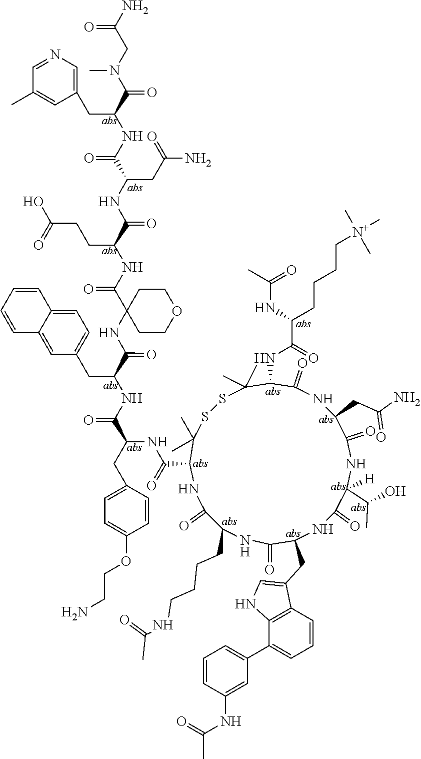 [SMILES: CC(=O)NCCCC[C@@H]1NC(=O)[C@H](Cc2c[nH]c3c(-c4cccc(NC(C)=O)c4)cccc23)NC(=O)[C@H]([C@@H](C)O)NC(=O)[C@H](CC(N)=O)NC(=O)[C@@H](NC(=O)[C@@H](CCCC[N+](C)(C)C)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cncc(C)c3)C(=O)N(C)CC(N)=O)CCOCC2)NC1=O] MeCO-k(Me)3-Pen(3)-N-T-7(3NAcPh)W-K(Ac)-Pen(3)-AEF-2Nal-THP-E-N- 5MePyridinAla-Sar-CONH2 |
| 536 | 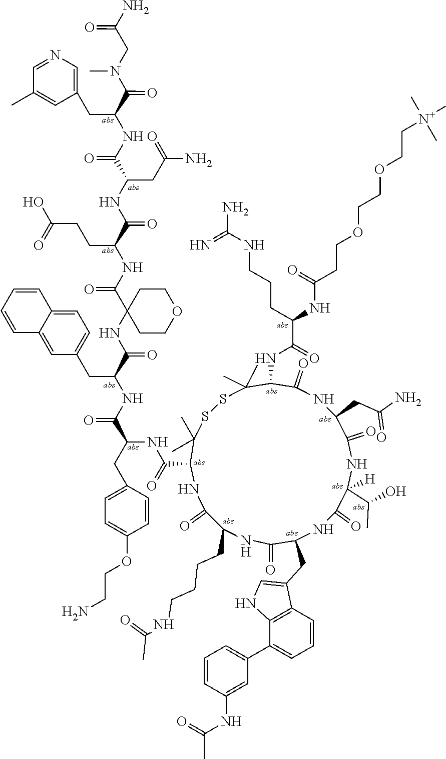 [SMILES: CC(=O)NCCCC[C@@H]1NC(=O)[C@H](Cc2c[nH]c3c(-c4cccc(NC(C)=O)c4)cccc23)NC(=O)[C@H]([C@@H](C)O)NC(=O)[C@H](CC(N)=O)NC(=O)[C@@H](NC(=O)[C@@H](CCCNC(=N)N)NC(=O)CCOCCOCC[N+](C)(C)C)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cncc(C)c3)C(=O)N(C)CC(N)=O)CCOCC2)NC1=O] cPEG3aCO-r-Pen(3)-N-T-7(3NAcPh)W-K(Ac)-Pen(3)-AEF-2Nal-THP-E-N- 5MePyridinAla-Sar-CONH2 |
| 537 | 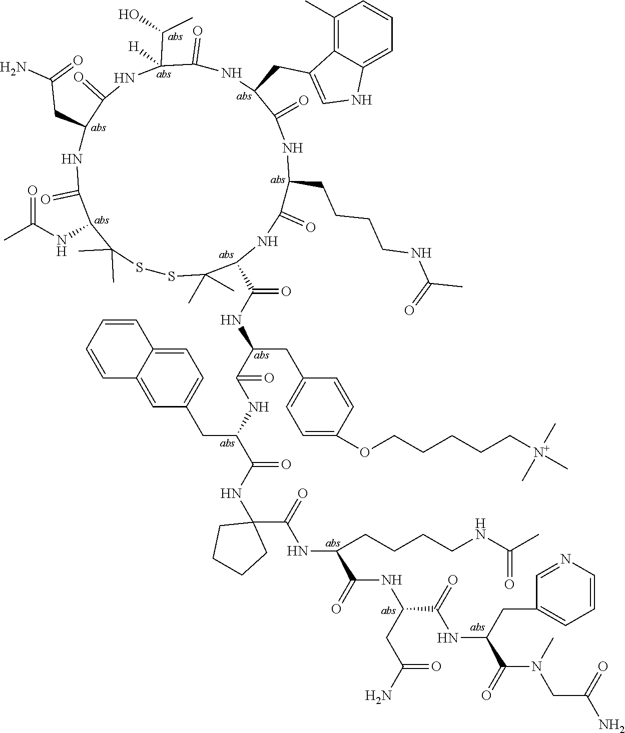 [SMILES: CC(=O)NCCCC[C@@H]1NC(=O)[C@H](Cc2c[nH]c3cccc(C)c23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(N)=O)CCCC2)NC1=O] MeCO-Pen(3)-N-T-7MeW-K(Ac)-Pen(3)-TMAPF-2Nal-Acpx-K(Ac)-N-3Pya-Sar- CONH2 |
| 538 | 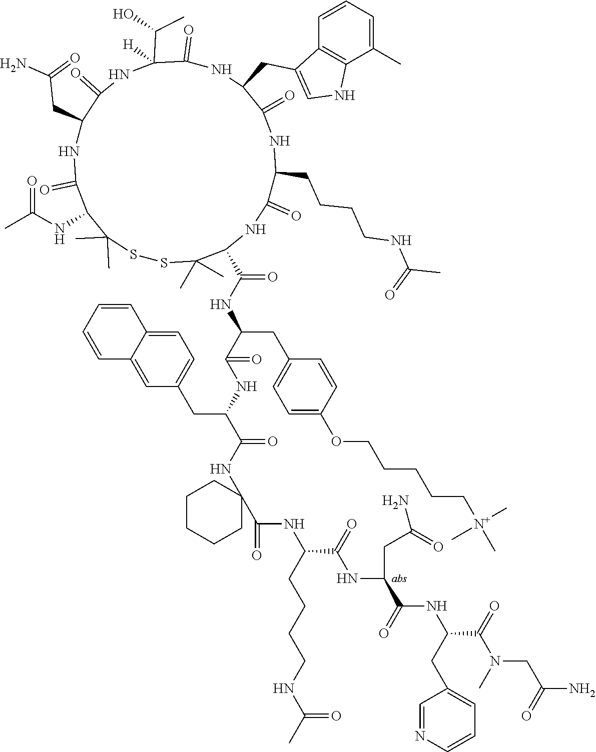 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(N)=O)CCCCC2)NC1=O] MeCO-Pen(3)-N-T-7MeW-K(Ac)-Pen(3)-TMAPF-2Nal-Achx-K(Ac)-N-3Pya-Sar- CONH2 |
| 539 | 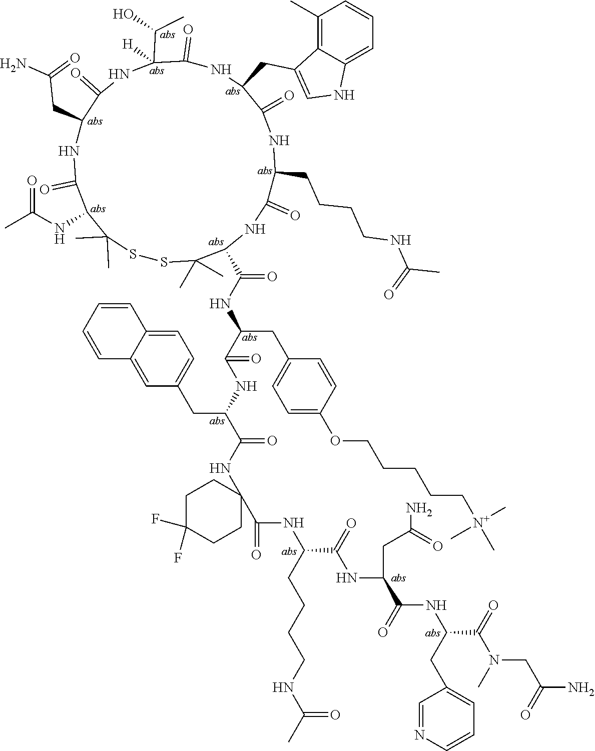 [SMILES: CC(=O)NCCCC[C@@H]1NC(=O)[C@H](Cc2c[nH]c3cccc(C)c23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(N)=O)CCC(F)(F)CC2)NC1=O] MeCO-Pen(3)-N-T-7MeW-K(Ac)-Pen(3)-TMAPF-2Nal-diFAchx-K(Ac)-N-3Pya- Sar-CONH2 |
| 540 | 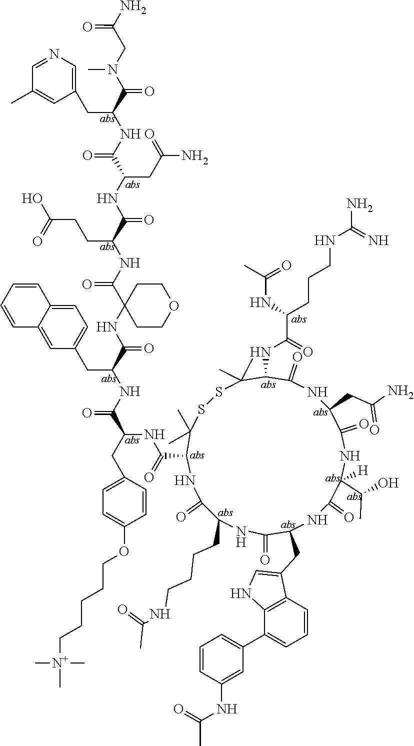 [SMILES: CC(=O)NCCCC[C@@H]1NC(=O)[C@H](Cc2c[nH]c3c(-c4cccc(NC(C)=O)c4)cccc23)NC(=O)[C@H]([C@@H](C)O)NC(=O)[C@H](CC(N)=O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cncc(C)c3)C(=O)N(C)CC(N)=O)CCOCC2)NC1=O] MeCO-r-Pen(3)-N-T-7(3NAcPh)W-K(Ac)-Pen(3)-TMAPF-2Nal-THP-E-N- 5MePyridinAla-Sar-CONH2 |
| 541 | 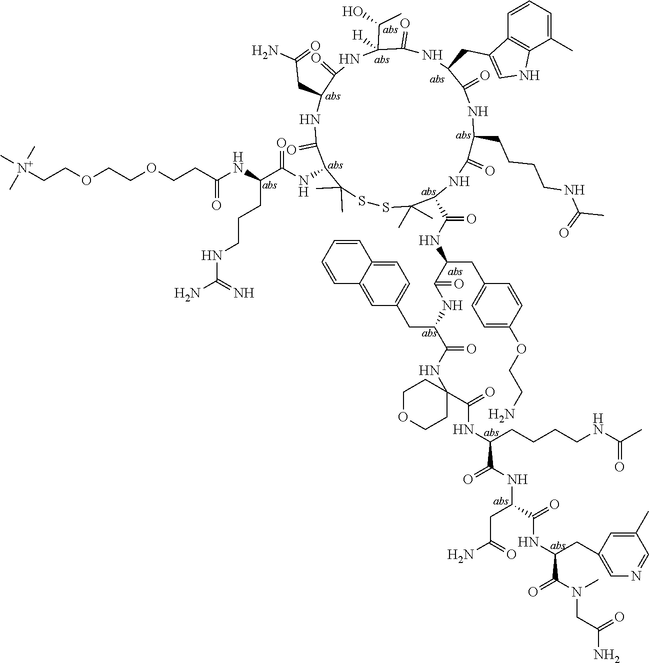 cPEG3aCO-r-Pen(3)-N-T-7MeW-K(Ac)-Pen(3)-AEF-2Nal-THP-K(Ac)-N- 5MePyridinAla-Sar-CONH2 |
| 542 | 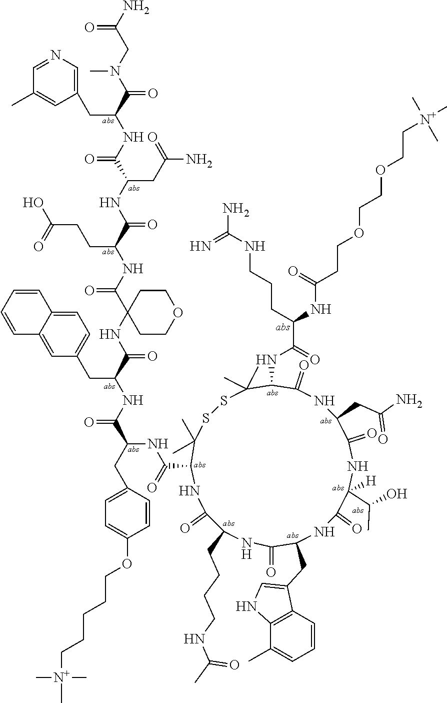 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(=O)CCOCCOCC[N+](C)(C)C)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cncc(C)c3)C(=O)N(C)CC(N)=O)CCOCC2)NC1=O] cPEG3aCO-r-Pen(3)-N-T-7MeW-K(Ac)-Pen(3)-TMAPF-2Nal-THP-E-N- 5MePyridinAla-Sar-CONH2 |
| 543 | 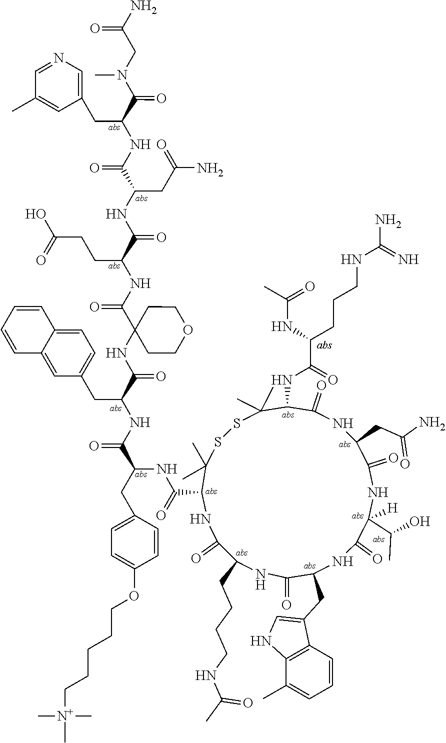 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cncc(C)c3)C(=O)N(C)CC(N)=O)CCOCC2)NC1=O] MeCO-r-Pen(3)-N-T-7MeW-K(Ac)-Pen(3)-TMAPF-2Nal-THP-E-N- 5MePyridinAla-Sar-CONH2 |
| 544 | 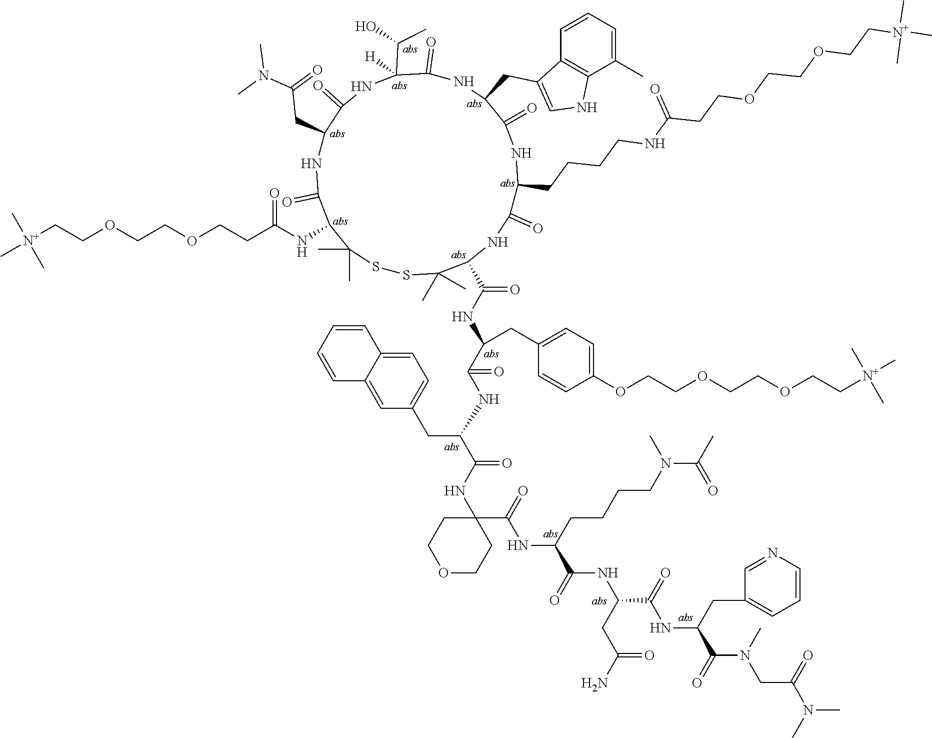 cPEG3aCO-Pen(3)-N(N(Me)2)-T-7MeW-K(cPEG3aCO)-Pen(3)-APEG3F-2Nal- THP-K(NMeAc)-N-3Pya-Sar-CON(Me)2 |
| 545 | 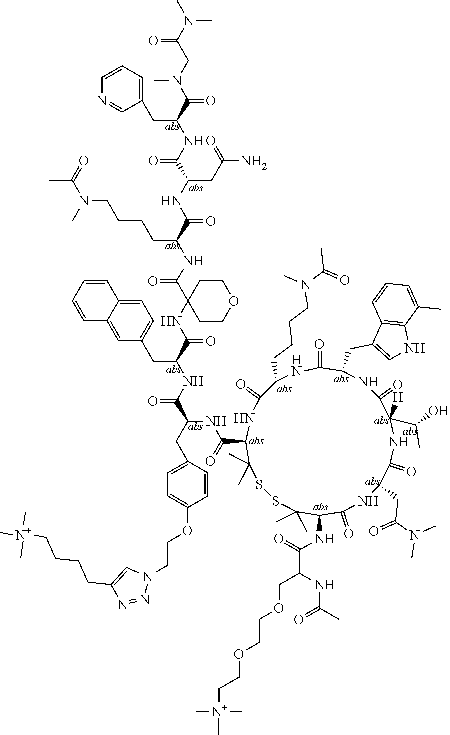 MeCO-APEG2Ser(RS)-Pen(3)-N(N(Me)2)-T-7MeW-K(NMeAc)-Pen(3)- Y(OTzlTMA4)-2Nal-THP-K(NMeAc)-N-3Pya-Sar-CON(Me)2 |
| 546 | 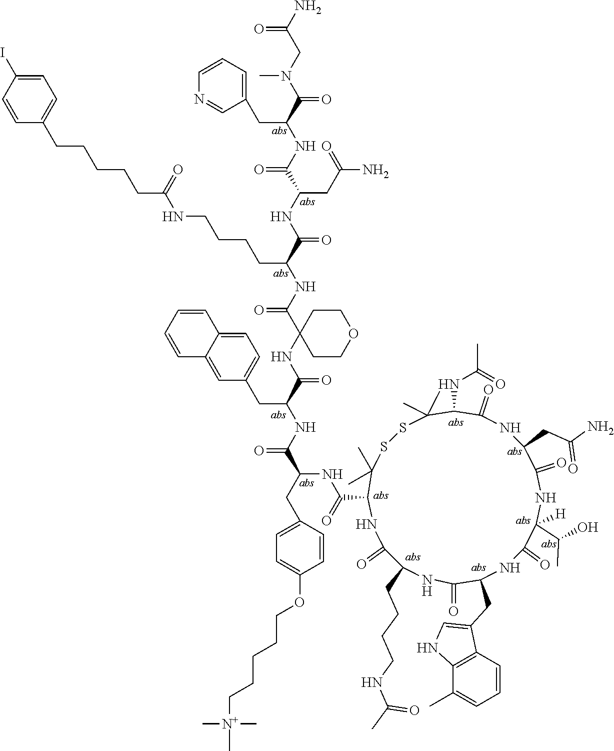 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCNC(=O)CCCCCc3ccc(I)cc3)C(=O)N[C@@H](CC(N)=O)C(=O)N[C@@H](Cc3cccnc3)C(=O)N(C)CC(N)=O)CCOCC2)NC1=O] MeCO-Pen(3)-N-T-7MeW-K(Ac)-Pen(3)-TMAPF-2Nal-THP-K(IPB)-N-3Pya-Sar- CONH2 |
| 547 | 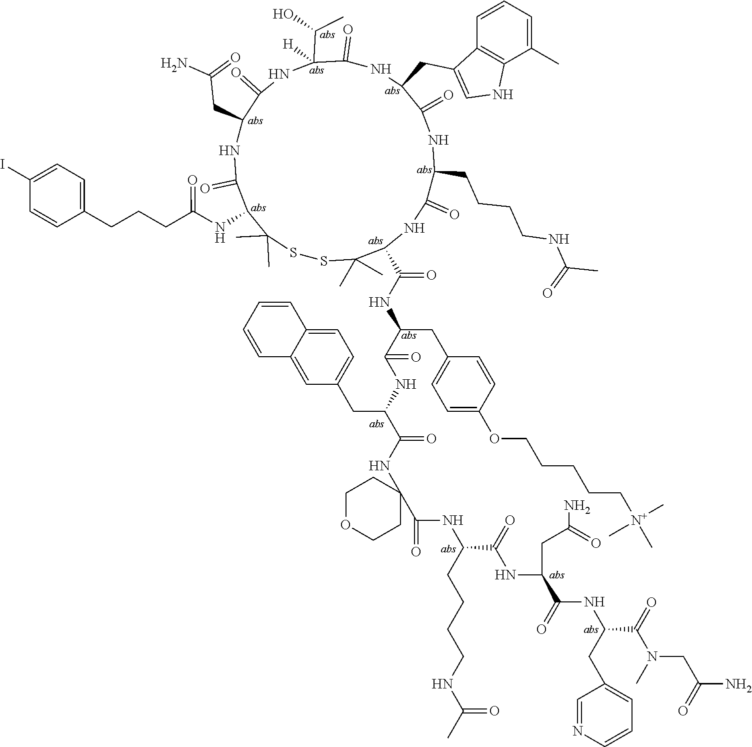 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)CCCc2ccc(I)cc2)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(N)=O)CCOCC2)NC1=O] IPBCO-Pen(3)-N-T-7MeW-K(Ac)-Pen(3)-TMAPF-2Nal-THP-K(Ac)-N-3Pya-Sar- CONH2 |
| 548 | 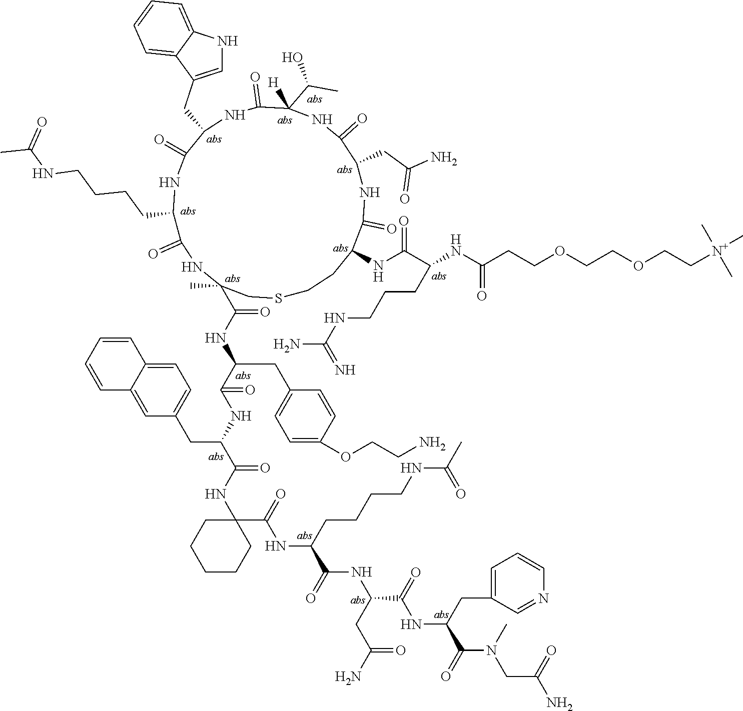 [SMILES: CC(=O)NCCCC[C@@H]1NC(=O)[C@H](Cc2c[nH]c3ccccc23)NC(=O)[C@H]([C@@H](C)O)NC(=O)[C@H](CC(N)=O)NC(=O)[C@@H](NC(=O)[C@@H](CCCNC(=N)N)NC(=O)CCOCCOCC[N+](C)(C)C)CCSC[C@@](C)(C(=O)N[C@@H](Cc2ccc(OCCN)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(N)=O)CCCCC2)NC1=O] cPEG3aCO-r-Abu(1)-N-T-W-K(Ac)-aMeC(1)-AEF-2Nal-THP-K(Ac-N-3Pya-Sar- CONH2 |
| 550 | 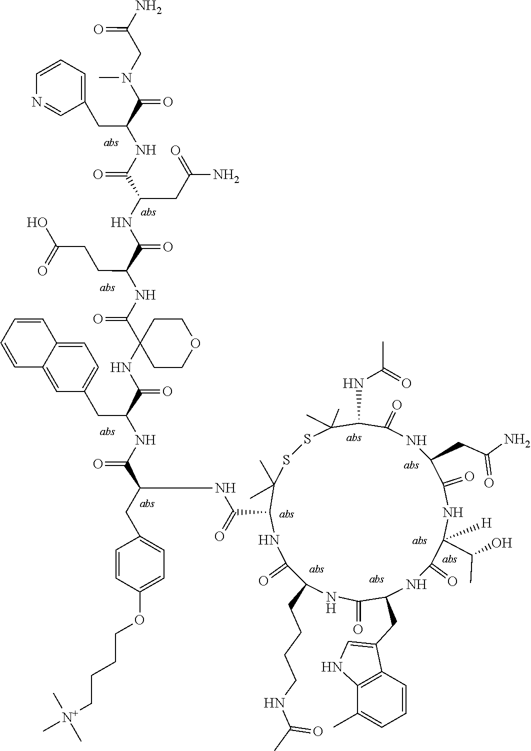 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCCC[N+](C)(C)C)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O] MeCO-Pen(3)-N-T-7MeW-K(Ac)-Pen(3)-TMA4F-2Nal-THP-N-3Pya-Sar-CONH2 |

551 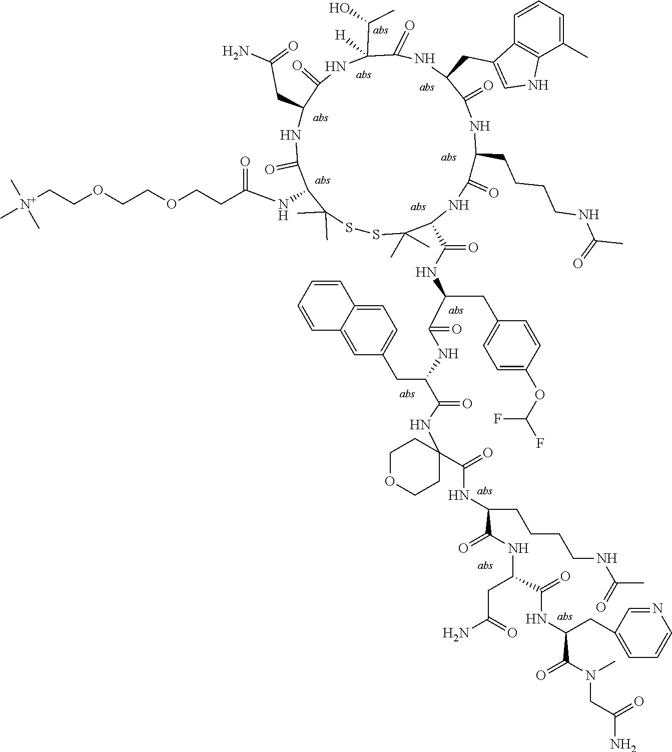 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)CCOCCOCC[N+](C)(C)C)C(C)(C)SSC(C)(C)[C@@H](C(=O)N[C@@H](Cc2ccc(OC(F)F)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(N)=O)CCOCC2)NC1=O] cPEG3aCO-Pen(3)-N-T-7MeW-K(Ac)-Pen(3)-YCF2H-2Nal-THP-K(Ac- N-3Pya-Sar-CONH2

| | |
|---|---|
| 552 | 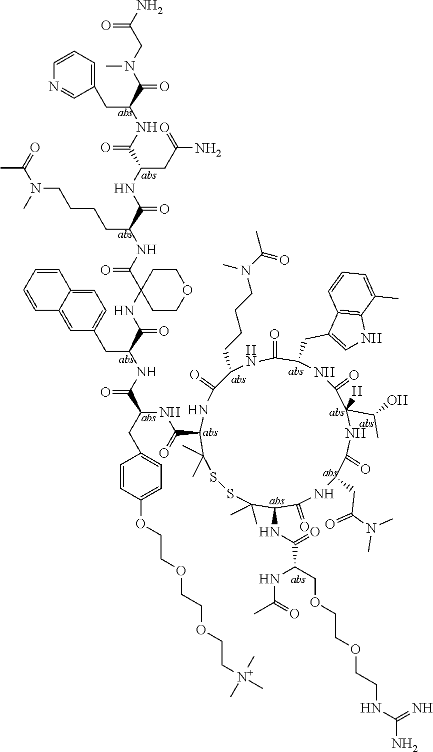 [SMILES: CC(=O)N[C@@H](COCCOCCNC(=N)N)C(=O)N[C@@H]1C(=O)N[C@@H](CC(=O)N(C)C)C(=O)N[C@@H]([C@@H](C)O)C(=O)N[C@@H](Cc2c[nH]c3c(C)cccc23)C(=O)N[C@@H](CCCCN(C)C(C)=O)C(=O)N[C@H](C(=O)N[C@@H](Cc2ccc(OCCOCCOCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(N)=O)CCOCC2)C(C)(C)SSC1(C)C] MeCO-gPEG2Ser-Pen(3)-N(N(Me)2)-T-7MeW-K(NMeAc)-Pen(3)-APEG3F-2Nal- THP-K(NMeAc)-N-3Pya-Sar-CON(Me)2 |
| 553 | 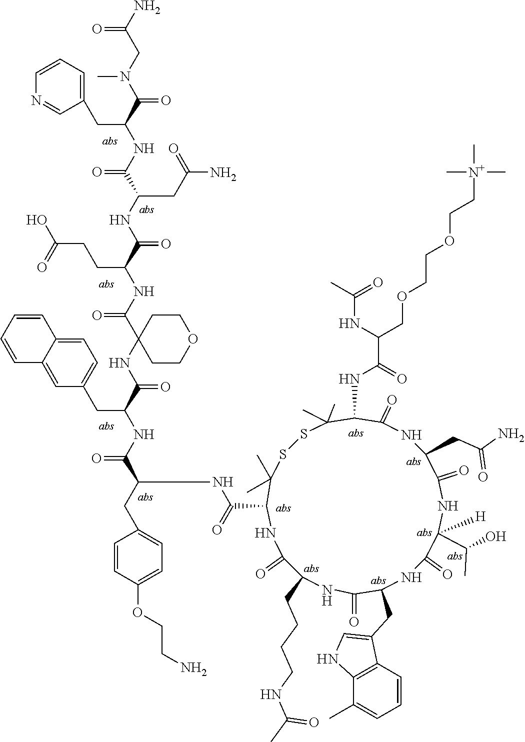 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)C(COCCOCC[N+](C)(C)C)NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O] MeCO-APEG2Ser(RS)-Pen(3)-N-T-7MeW-K(Ac)-Pen(3)-AEF-2Nal-THP-E-N- 3Pya-Sar-CONH2 |
| 554 | 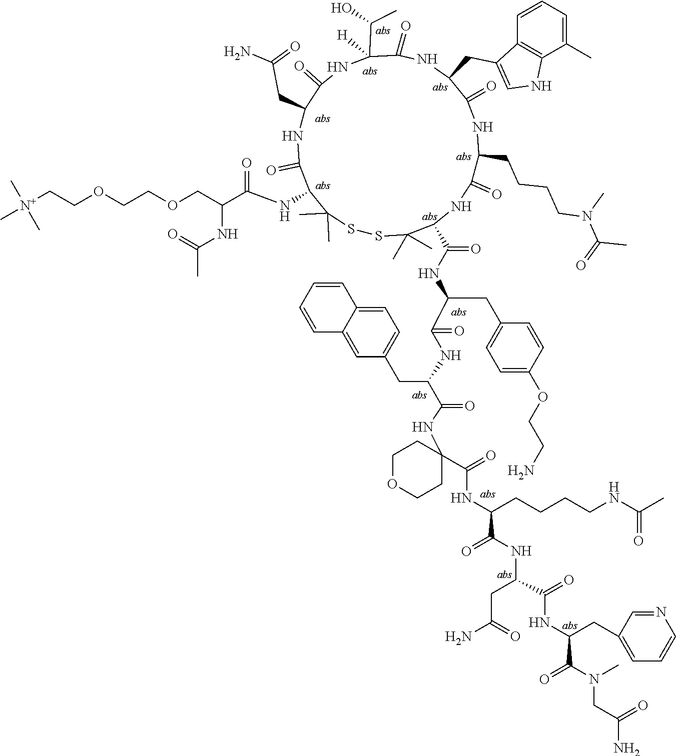 [SMILES: CC(=O)NCCCC[C@H](NC(=O)C1(NC(=O)[C@H](Cc2ccc3ccccc3c2)NC(=O)[C@H](Cc2ccc(OCCN)cc2)NC(=O)[C@H]2NC(=O)[C@H](CCCCN(C)C(C)=O)NC(=O)[C@H](Cc3c[nH]c4c(C)cccc34)NC(=O)[C@H]([C@@H](C)O)NC(=O)[C@H](CC(N)=O)NC(=O)[C@@H](NC(=O)C(COCCOCC[N+](C)(C)C)NC(C)=O)C(C)(C)SSC2(C)C)CCOCC1)C(=O)N[C@@H](CC(N)=O)C(=O)N[C@@H](Cc1cccnc1)C(=O)N(C)CC(N)=O] MeCO-APEG2Ser(RS)-Pen(3)-N-T-7MeW-K(Ac)-Pen(3)-AEF-2Nal-THP-K(Ac)- N-3Pya-Sar-CONH2 |
| 555 | 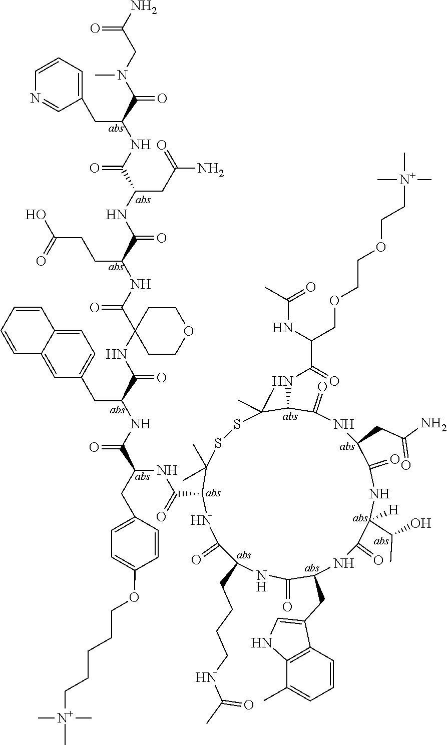 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)C(COCCOCC[N+](C)(C)C)NC(C)=O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O] MeCO-APEG2Ser(RS)-Pen(3)-N-T-7MeW-K(Ac)-Pen(3)-TMAPF-2Nal-THP-E-N- 3Pya-Sar-CONH2 |
| 556 | 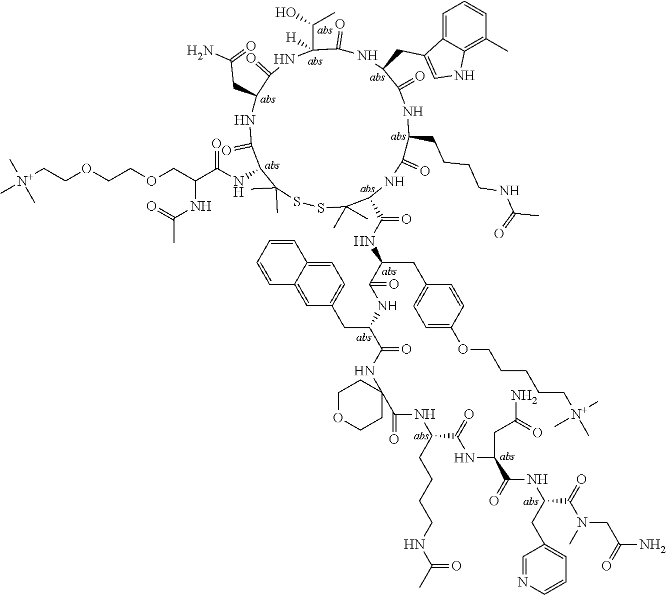 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)C(COCCOCC[N+](C)(C)C)NC(C)=O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(N)=O)CCOCC2)NC1=O] MeCO-APEG2Ser(RS)-Pen(3)-N-T-7MeW-K(Ac)-Pen(3)-TMAPF-2Nal-THP- K(Ac)-N-3Pya-Sar-CONH2 |
| 557 | 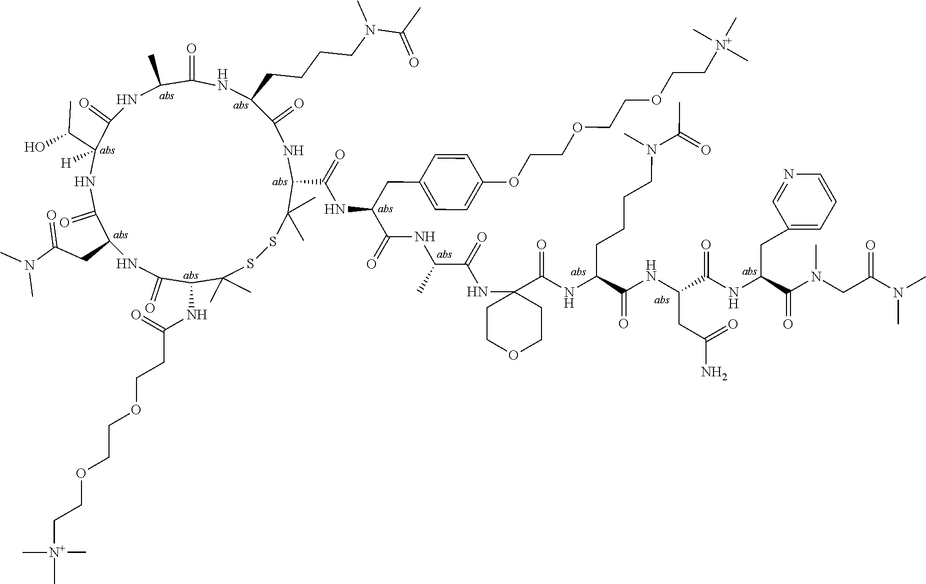 cPEG3aCO-Pen(3)-N(N(Me)2)-T-A-K(NMeAc)-Pen(3)-APEG3F-A-THP- K(NMeAc)-N-3Pya-Sar-CON(Me)2 |
| 558 | 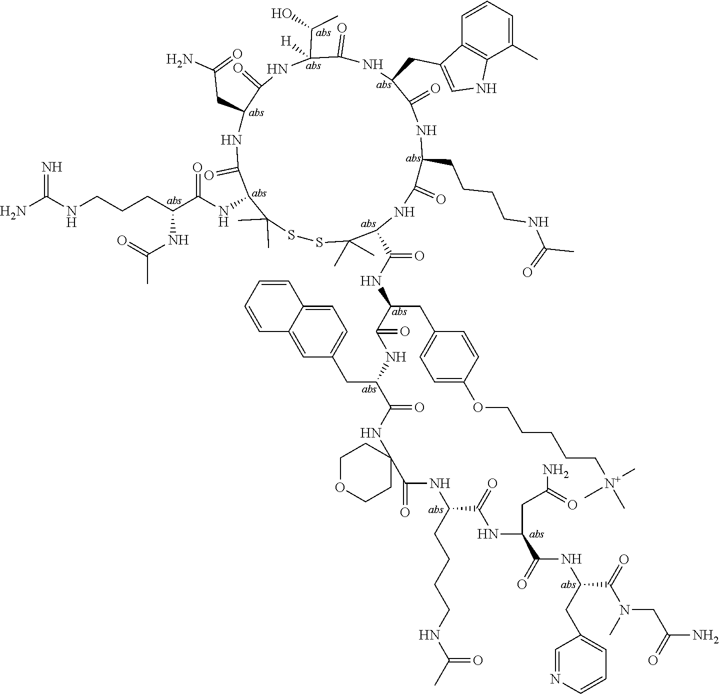 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(=O)[C@@H](CCCNC(=N)N)NC(C)=O)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(N)=O)CCOCC2)NC1=O] MeCO-r-Pen(3)-N-T-7MeW-K(Ac)-Pen(3)-TMAPF-2Nal-THP-K(Ac)-N-3Pya-Sar- CONH2 |
| 559 | 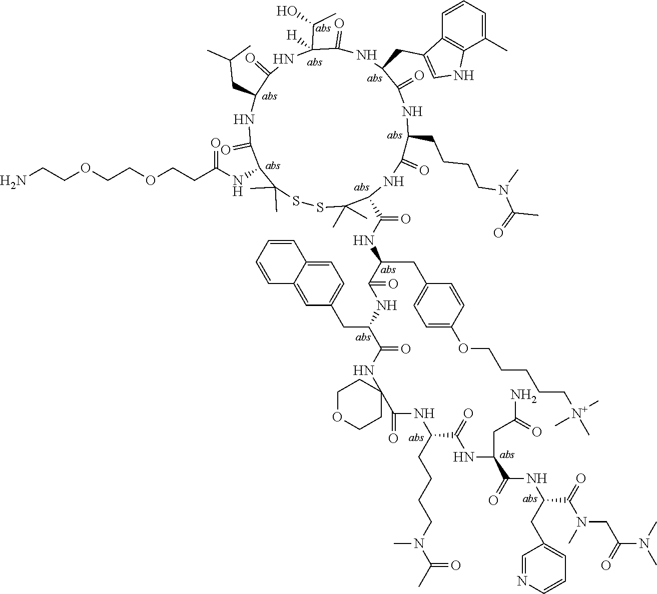 [SMILES: CC(=O)N(C)CCCC[C@@H]1NC(=O)[C@H](Cc2c[nH]c3c(C)cccc23)NC(=O)[C@H]([C@@H](C)O)NC(=O)[C@H](CC(C)C)NC(=O)[C@@H](NC(=O)CCOCCOCCN)C(C)(C)SSC(C)(C)[C@@H](C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCN(C)C(C)=O)C(=O)N[C@@H](CC(N)=O)C(=O)N[C@@H](Cc3cccnc3)C(=O)N(C)CC(=O)N(C)C)CCOCC2)NC1=O] cPEG3AmCO-Pen(3)-N(N(Me)2)-T-7MeW-K(NMeAc)-Pen(3)-TMAPF-2Nal- THP-K(NMeAc)-N-3Pya-Sar-CON(Me)2 |
| 560 | 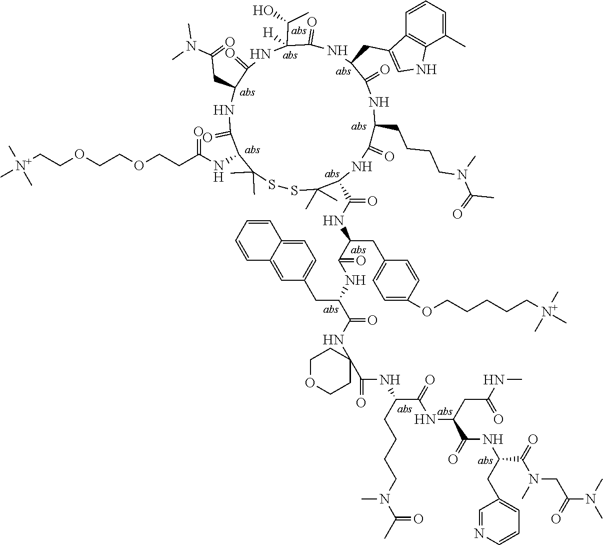 cPEG3aCO-Pen(3)-N(N(Me)2)-T-7MeW-K(NMeAc)-Pen(3)-TMAPF-2Nal-THP- k(NMeAc)-N(NMe)-3Pya-Sar-CON(Me)2 |
| 561 | 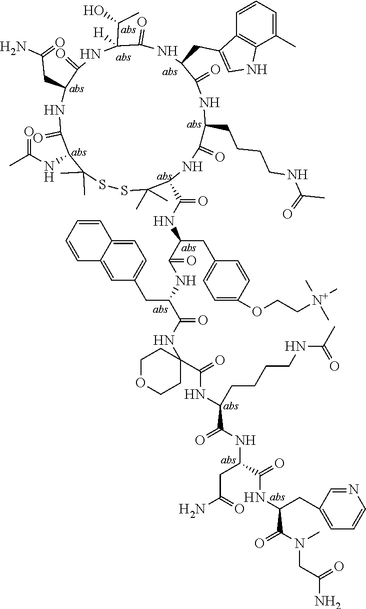 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCC[N+](C)(C)C)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(N)=O)CCOCC2)NC1=O] MeCO-Pen(3)-N-T-7MeW-K(Ac)-pen(3)-AEF(NMe3)-2Nal-THP-K(Ac)-N-3Pya- Sar-CONH2 |
| 562 | 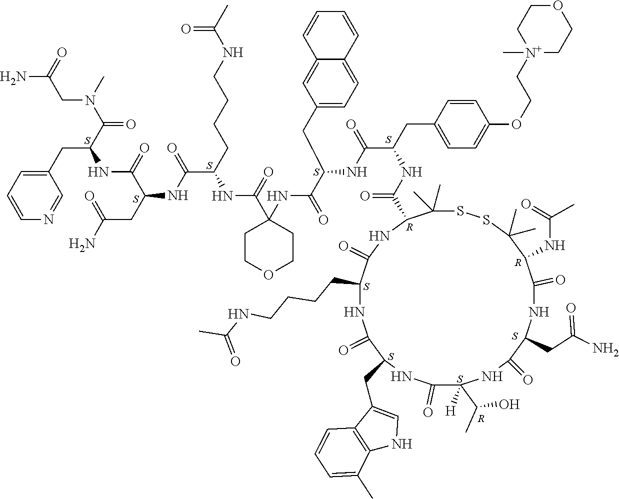 MeCO-Pen(3)-N-T-7MeW-K(Ac)-Pen(3)-MMoEF-2Nal-THP-K(Ac)-N-3Pya-Sar- CONH2 |
| 563 | 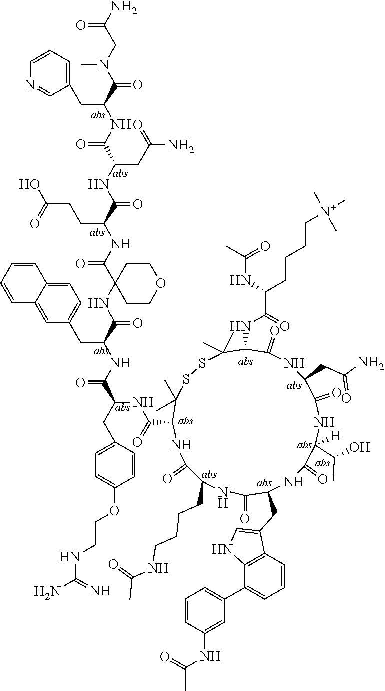 [SMILES: CC(=O)NCCCC[C@@H]1NC(=O)[C@H](Cc2c[nH]c3c(-c4cccc(NC(C)=O)c4)cccc23)NC(=O)[C@H]([C@@H](C)O)NC(=O)[C@H](CC(N)=O)NC(=O)[C@@H](NC(=O)[C@@H](CCCC[N+](C)(C)C)NC(C)=O)C(C)(C)SSC(C)(C)[C@@H](C(=O)N[C@@H](Cc2ccc(OCCNC(=N)N)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O] MeCO-k(Me)3-Pen(3)-N-T-7(3NAcPh)W-K(Ac)-Pen(3)-AEF(G)-2Nal-THP-E-N- 3Pya-Sar-CONH2 |
| 564 | 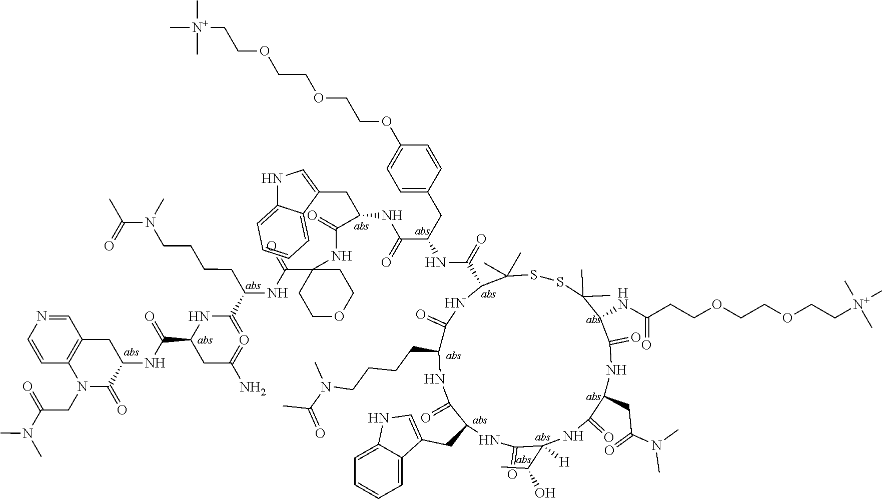 cPEG3aCO-Pen(3)-N(N(Me)2)-T-W-K(NMeAc)-Pen(3)-APEG3F-W-THP- K(NMeAc)-N-3Pya-Sar-CON(Me)2 |
| 565 | 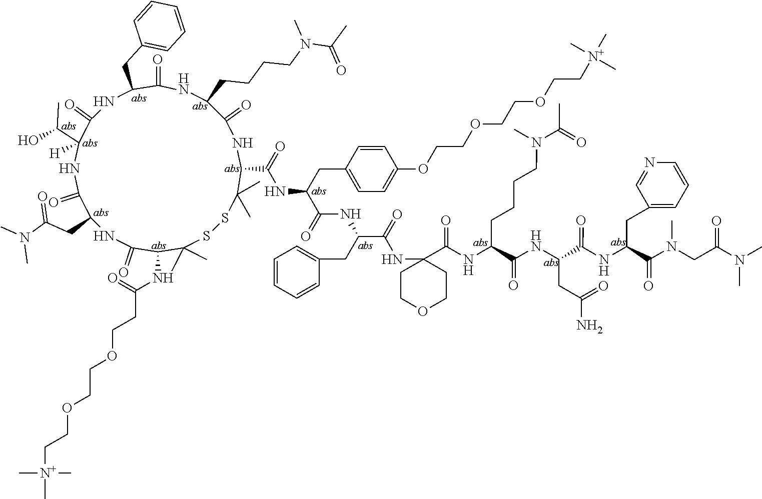 cPEG3aCO-Pen(3)-N(N(Me)2)-T-F-K(NMeAc)-Pen(3)-APEG3F-F-THP- K(NMeAc)-N-3Pya-Sar-CON(Me)2 |
| 566 | 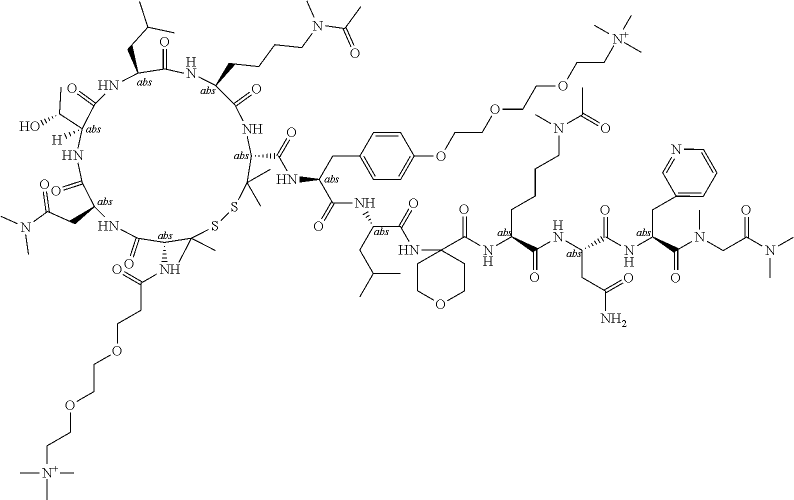 cPEG3aCO-Pen(3)-N(N(Me)2)-T-L-K(NMeAc)-Pen(3)-APEG3F-L-THP- K(NMeAc)-N-3Pya-Sar-CON(Me)2 |
| 567 | 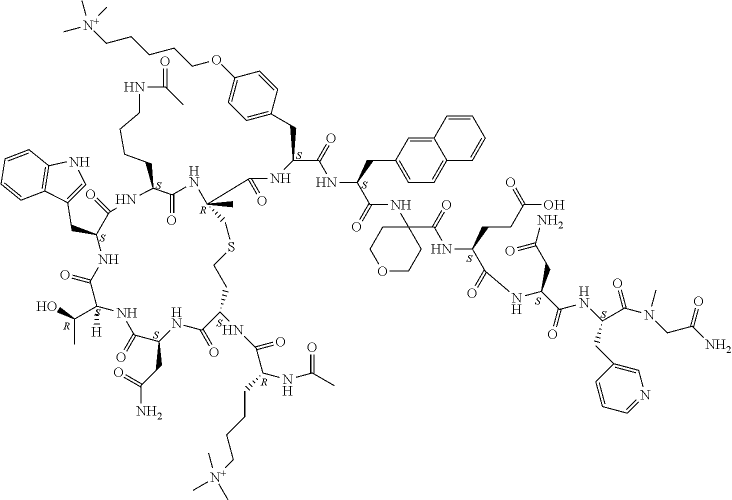 MeCO-k(Me)3-Abu(1)-N-T-W-K(Ac)-aMeC(1)-TMAPF-2Nal-THP-E-N-3Pya-Sar- CONH2 |
| 568 | 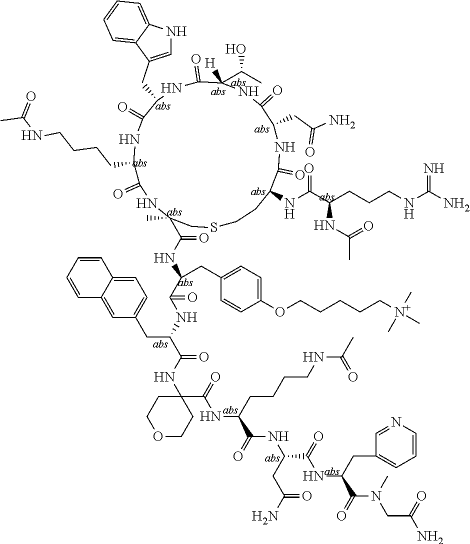 [SMILES: CC(=O)NCCCC[C@@H]1NC(=O)[C@H](Cc2c[nH]c3ccccc23)NC(=O)[C@H]([C@@H](C)O)NC(=O)[C@H](CC(N)=O)NC(=O)[C@@H](NC(=O)[C@@H](CCCNC(=N)N)NC(C)=O)CCSC[C@@](C)(C(=O)N[C@@H](Cc2ccc(OCCCCC[N+](C)(C)C)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(N)=O)CCOCC2)NC1=O] MeCO-r-Abu(1)-N-T-W-K(Ac)-aMeC(1)-TMAPF-2Nal-THP-K(Ac)-N-3Pya-Sar- CONH2 |
| 569 | 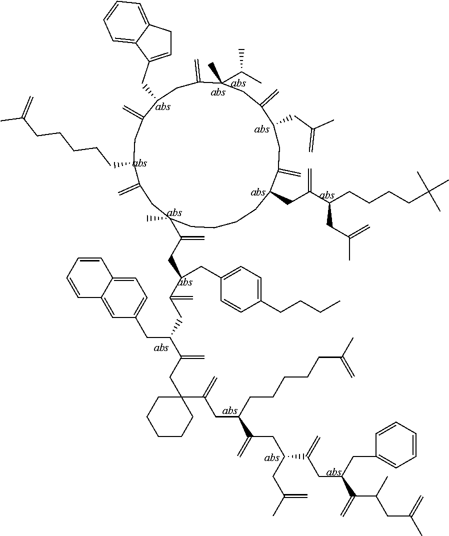 MeCO-k(Me)3-Abu(1)-N-T-W-K(Ac)-aMeC(1)-AEF-2Nal-THP-K(Ac)-N-3Pya- Sar-CONH2 |
| 570 | 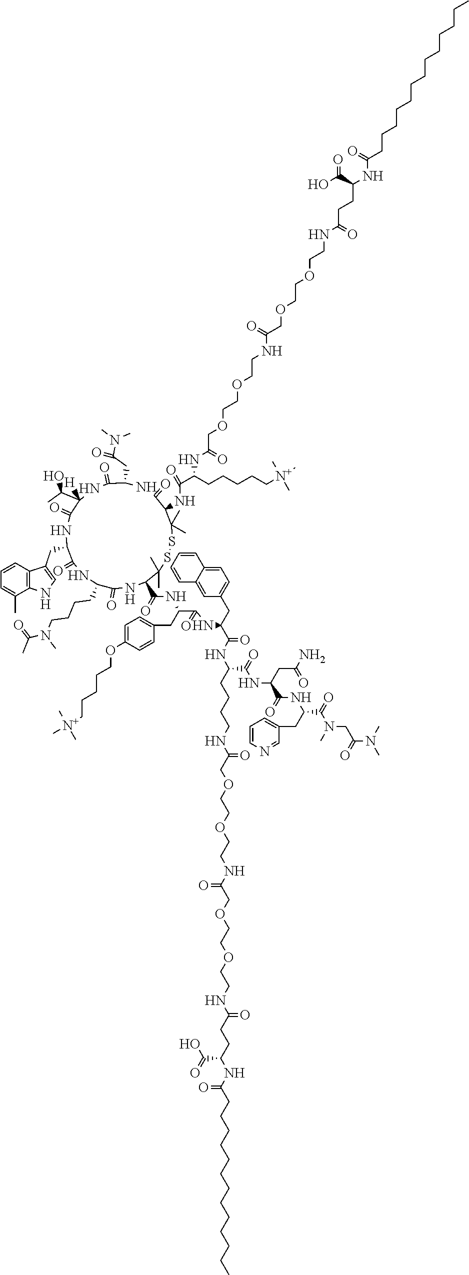 [SMILES: CCCCCCCCCCCCCC(=O)N[C@@H](CCC(=O)NCCOCCOCC(=O)NCCOCCOCC(=O)NCCCC[C@H](NC(=O)[C@H](Cc1ccc2ccccc2c1)NC(=O)[C@H](Cc1ccc(OCCCCC[N+](C)(C)C)cc1)NC(=O)[C@H]1NC(=O)[C@H](CCCCN(C)C(C)=O)NC(=O)[C@H](Cc2c[nH]c3c(C)cccc23)NC(=O)[C@H]([C@@H](C)O)NC(=O)[C@H](CC(=O)N(C)C)NC(=O)[C@@H](NC(=O)[C@@H](CCCCC[N+](C)(C)C)NC(=O)COCCOCCNC(=O)COCCOCCNC(=O)CC[C@H](NC(=O)CCCCCCCCCCCCC)C(=O)O)C(C)(C)SSC1(C)C)C(=O)N[C@@H](CC(N)=O)C(=O)N[C@@H](Cc1cccnc1)C(=O)N(C)CC(=O)N(C)C)C(=O)O] C14gEPEG2PEG2CO-hk(Me)3-Pen(3)-N(N(Me)2)-T-7MeW-K(NMeAc)-Pen(3)- TMAPF-2Nal-K(PEG2PEG2gEC14)-N-3Pya-Sar-CON(Me)2 |
| 571 | 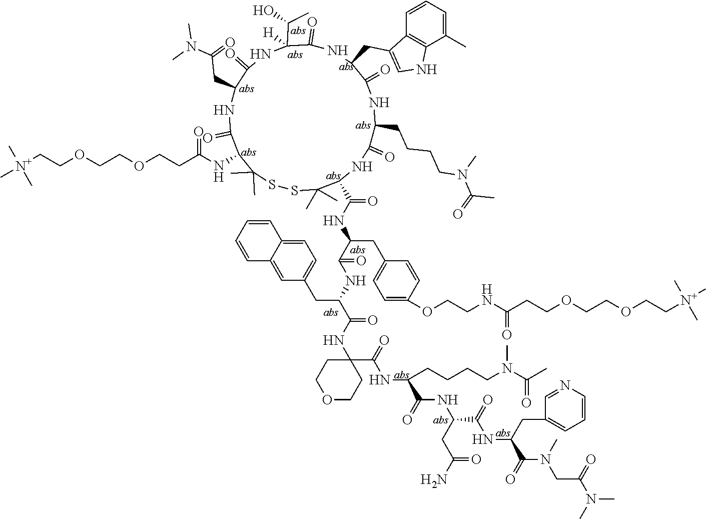 cPEG3aCO-Pen(3)-N(N(Me)2)-T-7MeW-K(NMeAc)-Pen(3)-AEF(cPEG3a)-2Nal- THP-K(NMeAc)-N-3Pya-Sar-CONMe2 |
| 572 | 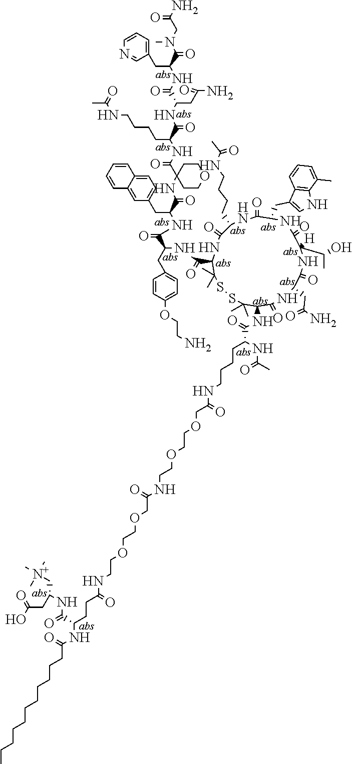 [SMILES: CCCCCCCCCCCC(=O)N[C@@H](CCC(=O)NCCOCCOCC(=O)NCCOCCOCC(=O)NCCCC[C@@H](NC(C)=O)C(=O)N[C@@H]1C(=O)N[C@@H](CC(N)=O)C(=O)N[C@@H]([C@@H](C)O)C(=O)N[C@@H](Cc2c[nH]c3c(C)cccc23)C(=O)N[C@@H](CCCCNC(C)=O)C(=O)N[C@H](C(=O)N[C@@H](Cc2ccc(OCCN)cc2)C(=O)N[C@@H](Cc2ccc3ccccc3c2)C(=O)NC2(C(=O)N[C@@H](CCCCNC(C)=O)C(=O)N[C@@H](CC(N)=O)C(=O)N[C@@H](Cc3cccnc3)C(=O)N(C)CC(N)=O)CCOCC2)C(C)(C)SSC1(C)C)C(=O)N[C@H](CC(=O)O)C[N+](C)(C)C] MeCO-canEG2PEG2gE(c)C12-Pen(3)-N-T-7MeW-K(Ac)-Pen(3)-AEF-2Nal-THP- K(Ac)-N-3Pya-Sar-CONH2 |
| 573 | 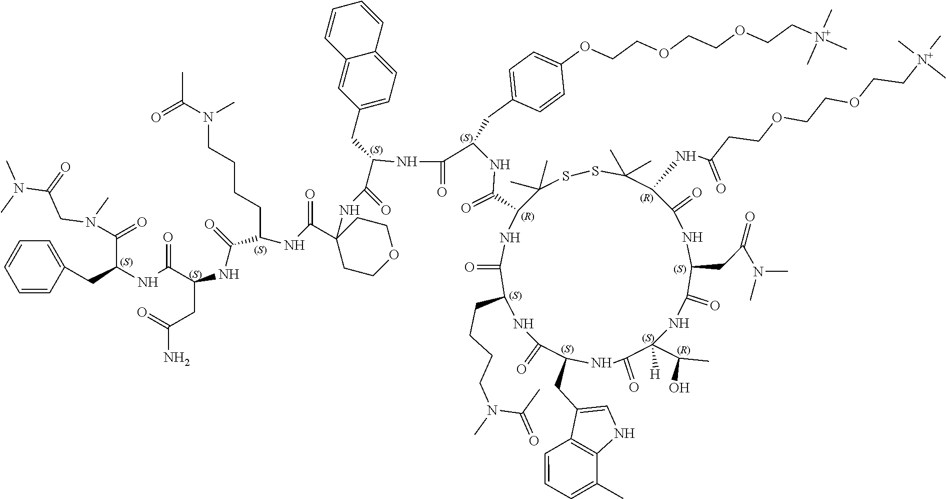 cPEG3aCO-Pen(3)-N(N(Me)2)-T-7MeW-K(NMeAc)-Pen(3)-APEG3F-2Nal-THP- K(NMeAc)-N-F-Sar-CON(Me)2 |
| 574 | 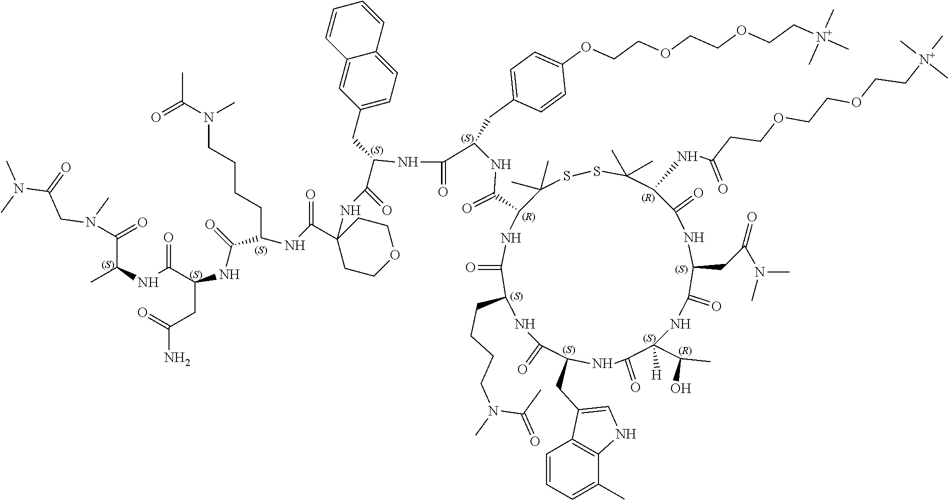 cPEG3aCO-Pen(3)-N(N(Me)2)-T-7MeW-K(NMeAc)-Pen(3)-APEG3F-2Nal-THP- K(NMeAc)-N-A-Sar-CON(Me)2 |

### SYNTHESIS

The compounds described herein may be synthesized by many techniques that are known to those skilled in the art. In certain aspects, monomer subunits are synthesized and purified using the techniques described in the accompanying Examples. In some aspects, the present invention provides a method of producing a compound (or monomer subunit thereof) of the invention, comprising chemically synthesizing a peptide having an amino acid sequence described herein, including but not limited to any of the amino acid sequences set forth in the compounds of Formula (I) to Formula (VI), Table IA, Table 11B, Table IC, Table ID, and Table IE herein. In some aspects, a portion of the peptide is recombinantly synthesized, instead of being chemically synthesized. In some aspects, methods of producing a compound further include cyclizing the compound precursor after the constituent subunits have been attached. In particular aspects, cyclization is accomplished via any of the various methods described herein.

Substituted tryptophans may be prepared by any suitable route. Preparation of certain substituted tryptophans including those substituted at the 7 position, such as 7-ethyl-L-tryptophans, are described in, for example WO 2021/146441 A1.

The present invention further describes synthesis of compounds described herein, such as the compounds of Formulae (I) to (X) and the compounds of Table 1A, Table 1A, Table 1B, Table 1C, Table 1D, and Table 1E. In some aspects, one or more of the amino acid residues or amino acid monomers are lipidated and then covalently attached to one another to form a compound of the invention. In some aspects, one or more of the amino acid residues or amino acid monomers are covalently attached to one another and lipidated at an intermediate oligomer stage before attaching additional amino acids and cyclization to form a compound of the invention. In some aspects, a cyclic peptide is synthesized and then lipidated to form a compound of the invention. Illustrative synthetic methods are described in the Examples.

The present invention further describes synthesis of compounds described herein, such as the compounds of Formulae (I) to Formula (X), and the compounds of Table 1A, Table 1B, Table 1C, Table 1D, and Table 1E. Illustrative synthetic methods are described in the Examples.

### IV. Pharmaceutical Compositions

The present invention relates to pharmaceutical composition which comprise an IL-23R inhibitor of the present invention. The present invention includes pharmaceutical compositions comprising one or more inhibitors of the present invention and a pharmaceutically acceptable carrier, diluent or excipient. The pharmaceutically acceptable carrier, diluent or excipient may be a solid, semi-solid or liquid filler, diluent, encapsulating material or formulation auxiliary of any type. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like.

The pharmaceutical compositions may be administered orally, parenterally, intracisternally, intravaginally, intraperitoneally, intrarectally, topically (as by powders, ointments, drops, suppository, or transdermal patch), by inhalation (such as intranasal spray), ocularly (such as intraocularly) or buccally. The term “parenteral” as used herein refers to modes of administration which include intravenous, intramuscular, intraperitoneal, intrasternal, subcutaneous, intradermal and intraarticular injection and infusion. Accordingly, in certain embodiments, the compositions are formulated for delivery by any of these routes of administration. A pharmaceutical composition may be formulated for and administered orally. A pharmaceutical composition may be formulated for and administered parenterally.

In a particular aspect, an IL-23R inhibitor of the present invention, is suspended in a sustained-release matrix. A sustained-release matrix, as used herein, is a matrix made of materials, usually polymers, which are degradable by enzymatic or acid-base hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid) polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. One embodiment of a biodegradable matrix is a matrix of one of either polylactide, polyglycolide, or polylactide co-glycolide (co-polymers of lactic acid and glycolic acid).

The IL-23R inhibitors of the present invention may be prepared and/or formulated as pharmaceutically acceptable salts or when appropriate in neutral form. Pharmaceutically acceptable salts are non-toxic salts of a neutral form of a compound that possess the desired pharmacological activity of the neutral form. These salts may be derived from inorganic or organic acids or bases. For example, a compound that contains a basic nitrogen may be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, 7-hydroxybutyrates, glycolates, tartrates, and mandelates. Lists of other suitable pharmaceutically acceptable salts are found in Remington: The Science and Practice of Pharmacy, 21^{st} Edition, Lippincott Wiliams and Wilkins, Philadelphia, Pa., 2006.

Examples of “pharmaceutically acceptable salts” of the compounds disclosed herein also include salts derived from an appropriate base, such as an alkali metal (for example, sodium, potassium), an alkaline earth metal (for example, magnesium), ammonium and NX₄⁺(wherein X is C₁-C₄ alkyl). Also included are base addition salts, such as sodium or potassium salts. Those skilled in the art will recognize that while quaternary amonium salts may be incorporated into the structure of the compound of Formula (I) - (VI), remaining basic residues may be combined with an acid form additional salt sites. Thus, a compound of Formula (I) - (VI) of the present invention may encompass quarternary ammonium salts at certain positions, but also acid addition salts at any basic site on the molecule.

The pesent invention relates to pharmaceutical compositions comprising an IL-23R inhibitor of the present invention or pharmaceutically acceptable salts, isomers, or a mixture thereof, in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds may increase resistance to metabolism, and thus may be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism,” Trends Pharmacol. Sci., 5(12):524-527 (1984). Such compounds are synthesized by means well known in the art, for example by employing starting materials in which one or more hydrogen atoms have been replaced by deuterium.

Examples of isotopes that can be incorporated into the disclosed compounds also include isotopes of hydrogen, carbon, nitrogen, oxygen, phosphorous, fluorine, chlorine, and iodine, such as ²H, ³H, C, ¹¹C, ¹³C, ¹⁴N, ¹⁵N, ¹⁵O, ¹⁷O, ¹⁸O, ³¹P, ³²P ³⁵S, ¹⁸F, ³⁶Cl, ¹²³I, and ¹²⁵I, respectively. Substitution with positron emitting isotopes, such as ¹¹C, ¹⁸F, ¹⁵0 and ¹³N, can be useful in Positron Emission Topography (PET) studies for examining substrate receptor occupancy. Isotopically-labeled compounds of Formula (I), can generally be prepared by conventional techniques known to those skilled in the art or by processes analogous to those described in the Examples as set out below using an appropriate isotopically-labeled reagent in place of the non-labeled reagent previously employed.

In certain aspects, pharmaceutical compositions for parenteral injection comprise pharmaceutically acceptable sterile aqueous or nonaqueous solutions, dispersions, suspensions or emulsions, or sterile powders, for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, β-cyclodextrin, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants. These compositions may also contain adjuvants such as preservative, wetting agents, emulsifying agents, and dispersing agents. Prolonged absorption of an injectable pharmaceutical form may be brought about by the inclusion of agents which delay absorption, such as aluminum monostearate and gelatin.

Injectable depot forms include those made by forming microencapsulated matrices of the peptide inhibitor in one or more biodegradable polymers such as polylactide-polyglycolide, poly(orthoesters), poly(anhydrides), and (poly)glycols, such as PEG.

Depending upon the ratio of peptide to polymer and the nature of the particular polymer employed, the rate of release of the peptide inhibitor can be controlled. Depot injectable formulations are also prepared by entrapping the peptide inhibitor in liposomes or microemulsions compatible with body tissues.

The injectable formulations may be sterilized, for example, by filtration through a bacterial-retaining filter, or by incorporating sterilizing agents in the form of sterile solid compositions which can be dissolved or dispersed in sterile water or other sterile injectable medium just prior to use.

Topical administration includes administration to the skin or mucosa, including surfaces of the lung and eye. Compositions for topical lung administration, including those for inhalation and intranasal, may involve solutions and suspensions in aqueous and non-aqueous formulations and can be prepared as a dry powder which may be pressurized or non-pressurized. In non-pressurized powder compositions, the active ingredient may be finely divided form may be used in admixture with a larger-sized pharmaceutically acceptable inert carrier comprising particles having a size, for example, of up to 100 micrometers in diameter. Suitable inert carriers include sugars such as lactose.

Alternatively, a pharmaceutical composition of the present invention may be pressurized and contain a compressed gas, such as nitrogen or a liquefied gas propellant. The liquefied propellant medium and indeed the total composition may be such that the active ingredient does not dissolve therein to any substantial extent. The pressurized composition may also contain a surface active agent, such as a liquid or solid non-ionic surface active agent or may be a solid anionic surface active agent. It is preferred to use the solid anionic surface active agent in the form of a sodium salt.

A further form of topical administration is to the eye. A peptide inhibitor of the present invention may be delivered in a pharmaceutically acceptable ophthalmic vehicle, such that the peptide inhibitor is maintained in contact with the ocular surface for a sufficient time period to allow the peptide inhibitor to penetrate the corneal and internal regions of the eye, as for example the anterior chamber, posterior chamber, vitreous body, aqueous humor, vitreous humor, cornea, iris/ciliary, lens, choroid/retina and sclera. The pharmaceutically acceptable ophthalmic vehicle may, for example, be an ointment, vegetable oil or an encapsulating material. Alternatively, the peptide inhibitors of the invention may be injected directly into the vitreous and aqueous humor.

Compositions for rectal or vaginal administration include suppositories which may be prepared by mixing the peptide inhibitors of this invention with suitable non-irritating excipients or carriers such as cocoa butter, polyethylene glycol or a suppository wax, which are solid at room temperature but liquid at body temperature and, therefore, melt in the rectum or vaginal cavity and release the active compound.

Peptide inhibitors of the present invention may also be administered in liposomes or other lipid-based carriers. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a peptide inhibitor of the present invention, stabilizers, preservatives, excipients, and the like. In certain embodiments, the lipids comprise phospholipids, including the phosphatidyl cholines (lecithins) and serines, both natural and synthetic. Methods to form liposomes are known in the art.

Pharmaceutical compositions suitable for parenteral administration in a method or use described herein may comprise sterile aqueous solutions and/or suspensions of the IL:-23R inhibitors made isotonic with the blood of the recipient, generally using sodium chloride, glycerin, glucose, mannitol, sorbitol, and the like.

The present invention provides a pharmaceutical composition for oral delivery. Compositions and peptide inhibitors of the present invention may be prepared for oral administration according to any of the methods, techniques, and/or delivery vehicles described herein. Further, one having skill in the art will appreciate that the peptide inhibitors of the instant invention may be modified or integrated into a system or delivery vehicle that is not disclosed herein, yet is well known in the art and compatible for use in oral delivery of peptides.

Formulations for oral administration may comprise adjuvants (e.g. resorcinols and/or nonionic surfactants such as polyoxyethylene oleyl ether and n- ether) to artificially increase the permeability of the intestinal walls, and/or enzymatic inhibitors (e.g. pancreatic trypsin inhibitors, diisopropylfluorophosphate (DFF) or trasylol) to inhibit enzymatic degradation. In certain embodiments, the peptide inhibitor of a solid-type dosage form for oral administration can be mixed with at least one additive, such as sucrose, lactose, cellulose, mannitol, trehalose, raffinose, maltitol, dextran, starches, agar, alginates, chitins, chitosans, pectins, gum tragacanth, gum arabic, gelatin, collagen, casein, albumin, synthetic or semisynthetic polymer, or glyceride. These formulations for oral administration can also contain other type(s) of additives, e.g., inactive diluting agent, lubricant such as magnesium stearate, paraben, preserving agent such as sorbic acid, ascorbic acid, alpha-tocopherol, antioxidants such as cysteine, disintegrators, binders, thickeners, buffering agents, pH adjusting agents, sweetening agents, flavoring agents or perfuming agents.

In particular aspects, oral dosage forms or unit doses compatible for use with the peptide inhibitors of the present invention may include a mixture of peptide inhibitor and nondrug components or excipients, as well as other non-reusable materials that may be considered either as an ingredient or packaging. Oral compositions may include at least one of a liquid, a solid, and a semi-solid dosage forms. In some embodiments, an oral dosage form is provided comprising an effective amount of peptide inhibitor, wherein the dosage form comprises at least one of a pill, a tablet, a capsule, a gel, a paste, a drink, a syrup, ointment, and suppository. In some instances, an oral dosage form is provided that is designed and configured to achieve delayed release of the peptide inhibitor in the subject's small intestine and/or colon.

Tablets may contain excipients, glidants, fillers, binders and the like. Aqueous compositions are prepared in sterile form, and when intended for delivery by other than oral administration generally will be isotonic. Compositions may optionally contain excipients such as those set forth in the “Handbook of Pharmaceutical Excipients” (1986). Excipients include ascorbic acid and other antioxidants, chelating agents such as EDTA, carbohydrates such as dextran, hydroxyalkylcellulose, hydroxyalkylmethylcellulose, stearic acid and the like. The pH of the compositions ranges from, for example, about 3 to about 11. The pH of the compositions may, for example, range from about 5 to about 7 or from about 7 to about 10.

An oral pharmaceutical composition of the present invention may comprise an IL-23R inhibitor of the present invention may comprise an enteric coating that is designed to delay release of the IL-23R inhibitor in the small intestine. The invention relates to a pharmaceutical composition that comprises an IL-23R inhibitor of the present invention and a protease inhibitor, such as aprotinin, in a delayed release pharmaceutical formulation.

Pharmaceutical compositions (e.g., oral pharmaceutical compositions) may comprise an enteric coat that is soluble in gastric juice at a pH of about 5.0 or higher. Such enteric coatings may comprise a polymer having dissociable carboxylic groups, such as derivatives of cellulose, including hydroxypropylmethyl cellulose phthalate, cellulose acetate phthalate and cellulose acetate trimellitate and similar derivatives of cellulose and other carbohydrate polymers.

An oral pharmaceutical composition comprising an IL-23R inhibitor of the present invention that comprises an IL-23R inhibitor may comprise an enteric coating that is designed to protect and release the pharmaceutical composition in a controlled manner within the subject's lower gastrointestinal system, and to avoid systemic side effects. In addition to enteric coatings, the peptide inhibitors of the instant invention may be encapsulated, coated, engaged or otherwise associated within any compatible oral drug delivery system or component. For example, in some embodiments an IL-23R inhibitor of the present invention is provided in a lipid carrier system comprising at least one of polymeric hydrogels, nanoparticles, microspheres, micelles, and other lipid systems.

To overcome peptide degradation of an IL-23R inhibitor of the present invention in the small intestine, the pharmaceutical compositions may comprise a hydrogel polymer carrier system in which a peptide inhibitor of the present invention is contained, whereby the hydrogel polymer protects the IL-23R inhibitor from proteolysis in the small intestine and/or colon. The an IL-23R inhibitor may further be formulated for compatible use with a carrier system that is designed to increase the dissolution kinetics and enhance intestinal absorption of the peptide. These methods include the use of liposomes, micelles and nanoparticles to increase GI tract permeation of peptides.

Various bioresponsive systems may also be combined with one or more an IL-23R inhibitors of the present invention to provide a pharmaceutical agent for oral delivery. For example, an IL-23R inhibitor of the present invention may be used in combination with a bioresponsive system, such as hydrogels and mucoadhesive polymers with hydrogen bonding groups (e.g., PEG, poly(methacrylic) acid [PMAA], cellulose, Eudragit®, chitosan and alginate) to provide a therapeutic agent for oral administration.

In certain aspects, pharmaceutical composition and formulations may include an IL-23R inhibitor of the present invention and one or more absorption enhancers, enzyme inhibitors, or mucoso adhesive polymers. In an embodiment, the absorption enhancer may be an intestinal permeation enhancer.

IL-23R inhibitors of the present invention may be formulated in a formulation vehicle, such as, e.g., emulsions, liposomes, microsphere or nanoparticles.

The present invention provides for a method for treating a subject with an IL-23R inhibitor of the present invention having an increased half-life. In one aspect, the present invention provides a peptide inhibitor having a half-life of at least several hours to one day in vitro or in vivo (e.g., when administered to a human subject) sufficient for daily (q.d.) or twice daily (b.i.d.) dosing of a therapeutically effective amount. In certain embodiments, the IL-23R inhibitor has a half-life of three days or longer sufficient for weekly (q.w.) dosing of a therapeutically effective amount. In certain embodiments, the IL-23R inhibitor has a half-life of eight days or longer sufficient for bi-weekly (b.i.w.) or monthly dosing of a therapeutically effective amount. In certain embodiments, the IL-23R inhibitor is derivatized or modified such that is has a longer half-life as compared to the underivatized or unmodified peptide inhibitor. In certain embodiments, the IL-23R inhibitor contains one or more chemical modifications to increase serum half-life.

When used in at least one of the treatments or delivery systems described herein, a peptide inhibitor of the present invention may be employed in pure form or, where such forms exist, in pharmaceutically acceptable salt form.

The total daily usage of the IL-23R inhibitor and compositions of the present invention can be decided by the attending physician within the scope of sound medical judgment. The specific therapeutically effective dose level for any particular subject will depend upon a variety of factors including: a) the disorder being treated and the severity of the disorder; b) activity of the specific compound employed; c) the specific composition employed, the age, body weight, general health, sex and diet of the patient; d) the time of administration, route of administration, and rate of excretion of the specific peptide inhibitor employed; e) the duration of the treatment; f) drugs used in combination or coincidental with the specific peptide inhibitor employed, and like factors well known in the medical arts.

In particular embodiments, the total daily dose of a IL-23R inhibitor of the present invention to be administered to a human or other mammal host in single or divided doses may be in amounts, for example, from 0.0001 to 300 mg/kg body weight daily or 1 to 300 mg/kg body weight daily.

The compositions may conveniently be presented in unit dosage form and can be prepared by any of the methods well known in the art of pharmacy. Techniques and compositions generally are foud in Remington's Pharmaceutical Sciences (Mack Publishing Co., Easton, PA). Such methods include the step of bringing into association the active ingredient with the carrier which constitutes one or more accessory ingredients. In general the compositions are prepared by uniformly and intimately bringing into association the active ingredient with liquid carriers or finely divided solid carriers or both, and then, if necessary, shaping the product.

Compositions suitable for oral administration can be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste. The active ingredient may also be administered as a buccal or sublingual formulation. Buccal or sublingual formulations may comprise an active ingredient in a matrix that releases the active ingredient for transport across the buccal and/or sublingual membranes. The buccal or sublingual formulation may further include a rate controlling matrix that releases the active compounds at a predetermined rate for transport across the buccal and/or sublingual membranes. The buccal or sublingual formulation may further include one or more compounds selected from the group consisting of (i) taste masking agents, (ii) enhancers, (iii) complexing agents, and mixtures thereof; and (iv) other pharmaceutically acceptable carriers and/or excipients. The enhancer may be a permeation enhancer.

A tablet is made by compression or molding, optionally with one or more accessory ingredients. Compressed tablets can be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets can optionally be coated or scored and optionally are formulated so as to provide slow or controlled release of the active ingredient therefrom.

### V. Non-Invasive Detection of Intestinal Inflammation

The IL-23R inhibitors of the present invention may be used for detection, assessment and diagnosis of intestinal inflammation by microPET imaging, wherein the peptide inhibitor is labeled with a chelating group or a detectable label, as part of a non-invasive diagnostic procedure. In certain embodiments, an IL-23R inhibitor of the present invention is conjugated with a bifunctional chelator. In certain embodiments, an IL-23R inhibitor of the present invention is radiolabeled. The labeled an IL-23R inhibitor is then administered to a subject orally or rectally. In certain embodiments, the IL-23R inhibitor is included in drinking water. Following uptake of the IL-23R inhibitor, microPET imaging may be used to visualize inflammation throughout the subject's bowels and digestive track.

VI. Methods of Treatments and/or Uses

The present invention relates to methods for treating a subject afflicted with a condition or indication associated with IL-23 or IL-23R (e.g., activation of the IL-23/IL-23R signaling pathway), wherein the method comprises administering to the subject an IL-23R inhibitor disclosed herein. A In one aspect, the present invention relates to a method for treating a subject afflicted with a condition or indication characterized by inappropriate, deregulated, or increased IL-23 or IL-23R activity or signaling, comprising administering to the individual a peptide inhibitor of the present invention in an amount sufficient to inhibit (partially or fully) binding of IL-23 to an IL-23R in the subject. The inhibition of IL-23 binding to IL-23R may occur in particular organs or tissues of the subject, e.g., the stomach, small intestine, large intestine/colon, intestinal mucosa, lamina propria, Peyer's Patches, mesenteric lymph nodes, or lymphatic ducts.

The present invention relates to methods comprising providing a peptide inhibitor described herein to a subject in need thereof. The subject in need thereof may be a subject that has been diagnosed with or has been determined to be at risk of developing a disease or disorder associated with IL-23/IL-23R. The subject may be a mammal. The subject may be, in particular, a human.

The disease or disorder to be treated by treatment with an IL-23R inhibitor of the present invention may be autoimmune inflammation and related diseases and disorders, such as multiple sclerosis, asthma, rheumatoid arthritis, inflammation of the gut, inflammatory bowel diseases (TBDs), juvenile TBD, adolescent TBD, Crohn's disease, ulcerative colitis, sarcoidosis, Systemic Lupus Erythematosus, ankylosing spondylitis (axial spondyloarthritis), psoriatic arthritis, or psoriasis. In particular, the disease or disorder may be psoriasis (e.g., plaque psoriasis, guttate psoriasis, inverse psoriasis, pustular psoriasis, Palmo-Plantar Pustulosis, psoriasis vulgaris, or erythrodermic psoriasis), atopic dermatitis, acne ectopica, ulcerative colitis, Crohn's disease, Celiac disease (nontropical Sprue), enteropathy associated with seronegative arthropathies, microscopic colitis, collagenous colitis, eosinophilic gastroenteritis/esophagitis, colitis associated with radio- or chemo-therapy, colitis associated with disorders of innate immunity as in leukocyte adhesion deficiency-1, chronic granulomatous disease, glycogen storage disease type 1b, Hermansky-Pudlak syndrome, Chediak-Higashi syndrome, Wiskott-Aldrich Syndrome, pouchitis, pouchitis resulting after proctocolectomy and ileoanal anastomosis, gastrointestinal cancer, pancreatitis, insulin-dependent diabetes mellitus, mastitis, cholecystitis, cholangitis, primary biliary cirrhosis, viral-associated enteropathy, pericholangitis, chronic bronchitis, chronic sinusitis, asthma, uveitis, or graft versus host disease.

The present invention relates to a method or use of an IL-23R inhibitor for treating an inflammatory disease in a subject that includes administering to the subject a therapeutically effective amount of an IL-23R inhibitor of the present invention or pharmaceutically acceptable solvate or salt thereof, or a composition disclosed herein comprising an IL-23 inhibitor of the present invention. In some aspects, the present invention provides a method of treating an inflammatory disease in a subject that includes administering to the subject a therapeutically effective amount of an IL-23R inhibitor of the present invention or pharmaceutically acceptable solvate or salt thereof, or a composition of the present invention. Suitable inflammatory diseases for treatment with a compound or pharmaceutically acceptable salt thereof, or a composition of the present invention, may include, but are not limited to inflammatory bowel disease (IBD), Crohn's disease (CD), ulcerative colitis (UC), psoriasis (PsO), or psoriatic arthritis (PsA) and the like. The inflammatory disease to be treated may be inflammatory bowel disease (IBD), Crohn's disease, or ulcerative colitis. The inflammatory disease to be treated may be selected from psoriasis, or psoriatic arthritis. The inflammatory disease to be treated may be psoriasis The inflammatory disease to be treated may be psoriatic arthritis. The inflammatory disease to be treated may be IBD.

The present invention relates to methods for treating an inflammatory disease in a subject in need thereof, comprising administering to the subject an IL-23R inhibitor disclosed herein (e.g., a peptide inhibitor or the IL-23R of Formula (I) to Formula (VI) or any of Tables 1A-1E. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory disease in a subject in need thereof, comprising administering to the subject an IL-23R inhibitor of Formulae I to X) or any of Tables 1A-1E. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods for treating an inflammatory disease in a subject in need thereof, comprising administering to the subject an IL-23R inhibitor of Formulae I to X) or any of Tables 1A-1E. The inflammatory disease may be IBD, Crohn's disease, or ulcerative colitis. In aspect, the IBD may be ulcerative colitis. In an aspect, the IBD may be Crohn's disease. In an aspect, the inflammatory disease may be psoriasis (PsO), or psoriatic arthritis (PsA).

The present invention relates to methods of inhibiting IL-23 binding to an IL-23R on a cell, comprising contacting the IL-23R with a peptide inhibitor of the receptor disclosed herein. The cell may be a mammalian cell. The method may be performed in vitro or in vivo. Inhibition of binding may be determined by a variety of routine experimental methods and assays known in the art.

The present invention relates to a method of selectively inhibiting IL-23 or IL-23R signaling (or the binding of IL-23 to IL-23R) in a subject (e.g., in a subject in need thereof), comprising providing to the subject a peptide inhibitor of the IL-23R described herein. The present invention includes and provides a method of selectively inhibiting IL-23 or IL-23R signaling (or the binding of IL-23 to IL-23R) in the GI tract of a subject (e.g., a subject in need thereof), comprising providing to the subject a peptide inhibitor of the IL-23R of the present invention by oral administration. The exposure of GI tissues (e.g., small intestine or colon) to the administered peptide inhibitor may be at least 10-fold, at least 20-fold, at least 50-fold, or at least 100-fold greater than the exposure (level) in the blood. In particular embodiments, the present invention includes a method of selectively inhibiting IL23 or IL23R signaling (or the binding of IL23 to IL23R) in the GI tract of a subject (e.g., a subject in need thereof), comprising providing to the subject a peptide inhibitor, wherein the peptide inhibitor does not block the interaction between IL-6 and IL-6R or antagonize the IL-12 signaling pathway. In a further related embodiment, the present invention includes a method of inhibiting GI inflammation and/or neutrophil infiltration to the GI, comprising providing to a subject in need thereof a peptide inhibitor of the present invention. In some embodiments, methods of the present invention comprise providing a peptide inhibitor of the present invention (i.e., a first therapeutic agent) to a subject (e.g., a subject in need thereof) in combination with a second therapeutic agent. In certain embodiments, the second therapeutic agent is provided to the subject before and/or simultaneously with and/or after the peptide inhibitor is administered to the subject. In particular embodiments, the second therapeutic agent is an anti-inflammatory agent. In certain embodiments, the second therapeutic agent is a non-steroidal anti-inflammatory drug, steroid, or immune modulating agent. In certain embodiments, the method comprises administering to the subject a third therapeutic agent. In certain embodiments, the second therapeutic agent is an antibody that binds IL-23 or IL-23R.

The present invention relates to methods of inhibiting IL-23 signaling by a cell, comprising contacting the IL-23R with a peptide inhibitor described herein. In certain embodiments, the cell is a mammalian cell. In particular embodiments, the method is performed in vitro or in vivo. In particular embodiments, the inhibition of IL-23 signaling may be determined by measuring changes in phospho-STAT3 levels in the cell.

In any of the foregoing methods, IL-23R inhibitor administration to a subject may be conducted orally, but other routes of administration are not excluded. Other routes of administration include, but are not limited to, parenteral, subcutaneous, intravenous, intramuscular, intraperitoneal, transdermal, topical, buccal or ocular routes. Dosages of a peptide inhibitor or the IL-23R described herein (e.g., a compound of Formulae (I) to (X) or any of Tables 1A-1E), or salt or solvate thereof to be administered to a subject may be determined by a person of skill in the art taking into account the the disease or condition being treated including its severity, and factors including the age weight, sex, and the like.

Exemplary dose ranges include, but are not limited to, from about 1 mg to about 1000 mg, or from about 1 mg to about 500 mg, from about 1 mg to about 100 mg, from about 10 mg to about 50 mg, from about 20 mg to about 40 mg, or from about 20 mg to about 30 mg. A dose range of a peptide inhibitor or the IL-23R described herein may be from about 600 mg to about 1000 mg. A dose range of a peptide inhibitor or the IL-23R described herein may be from about 300 mg to about 600 mg. A dose range of a peptide inhibitor or the IL-23R described herein may be from about 5 mg to about 300 mg. A dose range of a peptide inhibitor or the IL-23R described herein may be from about 25 mg to about 150 mg. A dose range of a peptide inhibitor or the IL-23R described herein may be from about 25 mg to about 100 mg. A dose range of a peptide inhibitor or the IL-23R described herein may be present in a dose range of from about 1 mg to about 100 mg. A dose range of a peptide inhibitor or the TL-23R described herein may be present in a dose range of from about 20 mg to about 40 mg. A dose range of a peptide inhibitor or the IL-23R described herein may be present in a dose range of from about 20 mg to about 30 mg.

### Examples

The following examples illustrate the invention. These examples are not intended to limit the scope of the present invention, but rather to provide guidance to the skilled artisan to prepare and use the compounds, compositions, and methods of the present invention. While particular aspects of the present invention are described, the skilled artisan will appreciate that various changes and modifications can be made without departing from the spirit and scope of the invention.

Some abbreviations useful in describing the invention are defined below in the following Table 2A and Table 2B.

TABLE 2A
Amino Acid Abbreviations

| Abbreviation | Definition |
|---|---|
| 2-Nal | L-2-naphthylalanine |
| 3Pya or 3Pal | L-3-pyridylalanine |
| 3pya | D-3-pyridylalanine |
| 4-amino-4-carboxy- tetrahydropyran or THP | 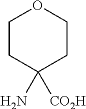 |
| | 4-amino-4-carboxy-tetrahydropyran |
| 4AmPhe or 4AmF | 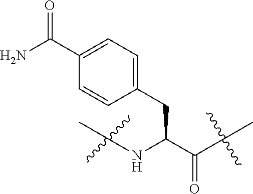 [SMILES: *N[C@@H](Cc1ccc(C(N)=O)cc1)C(*)=O] |
| Achx(diF) or 4diFAchx | 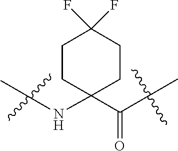 [SMILES: *NC1(C(*)=O)CCC(F)(F)CC1] |
| 4diFPro | 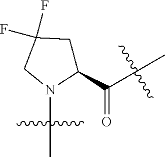 [SMILES: *C(=O)[C@@H]1CC(F)(F)CN1*] |
| 4DMPEF | 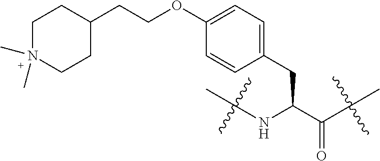 |
| 4DMPzEF | 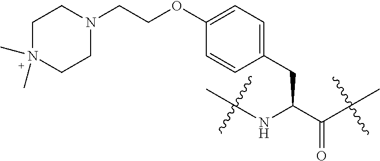 |
| 4TMABYF | 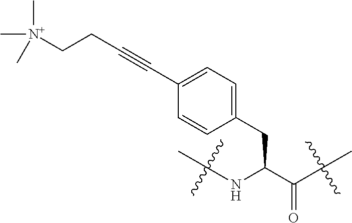 [SMILES: *N[C@@H](Cc1ccc(C#CCC[N+](C)(C)C)cc1)C(*)=O] |
| 5CF33Pya | 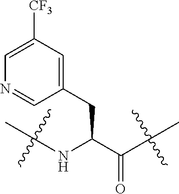 [SMILES: *N[C@@H](Cc1cncc(C(F)(F)F)c1)C(*)=O] |
| 5MePyridinAla | 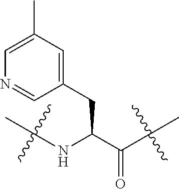 [SMILES: *N[C@@H](Cc1cncc(C)c1)C(*)=O] |
| 7(3NAcPh)W | 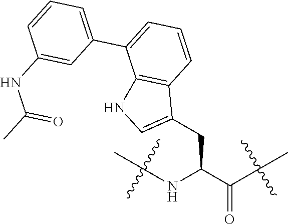 [SMILES: *N[C@@H](Cc1c[nH]c2c(-c3cccc(NC(C)=O)c3)cccc12)C(*)=O] |
| 7CF3W | 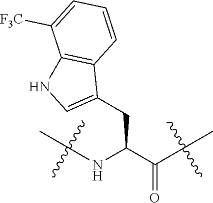 [SMILES: *N[C@@H](Cc1c[nH]c2c(C(F)(F)F)cccc12)C(*)=O] |
| 7MeW or W(7-Me) or | 7-methyl-L-tryptophan |
| Trp(7-Me) | |
| NMe7MeW | 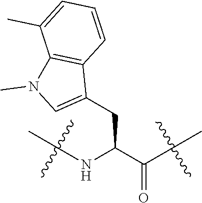 [SMILES: *N[C@@H](Cc1cn(C)c2c(C)cccc12)C(*)=O] |
| A | L-alanine |
| Abu | 2-aminobutyric acid |
| AAMPhe or Paf(Ac) | 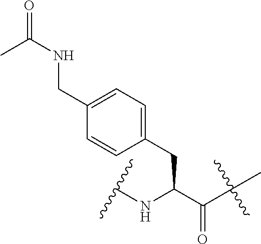 [SMILES: *N[C@@H](Cc1ccc(CNC(C)=O)cc1)C(*)=O] |
| ACHMF | 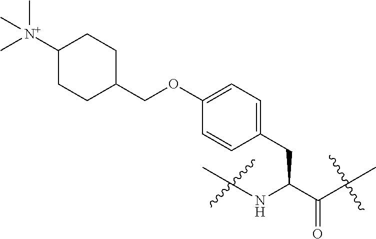 [SMILES: *N[C@@H](Cc1ccc(OCC2CCC([N+](C)(C)C)CC2)cc1)C(*)=O] |
| ACHMF(R*,S*) | 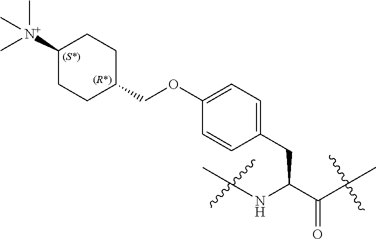 [SMILES: *N[C@@H](Cc1ccc(OC[C@H]2CC[C@H]([N+](C)(C)C)CC2)cc1)C(*)=O] |
| ACHMF(S*,S*) | 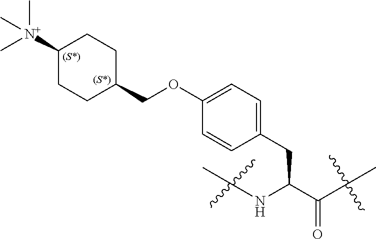 [SMILES: *N[C@@H](Cc1ccc(OC[C@H]2CC[C@@H]([N+](C)(C)C)CC2)cc1)C(*)=O] |
| ACHMF | 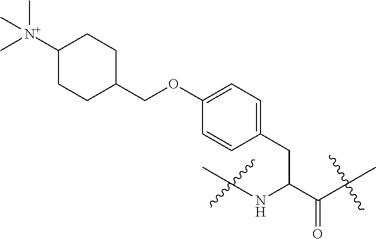 [SMILES: *NC(Cc1ccc(OCC2CCC([N+](C)(C)C)CC2)cc1)C(*)=O] |
| ACHMF(S*) | 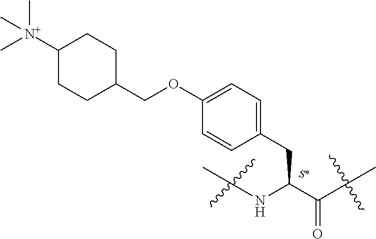 [SMILES: *N[C@@H](Cc1ccc(OCC2CCC([N+](C)(C)C)CC2)cc1)C(*)=O] |
| ACHMF(R*) | 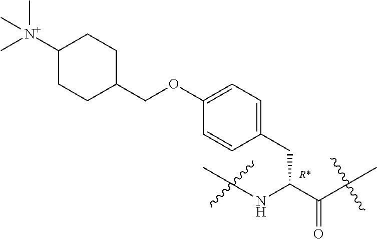 [SMILES: *N[C@H](Cc1ccc(OCC2CCC([N+](C)(C)C)CC2)cc1)C(*)=O] |
| Achx | 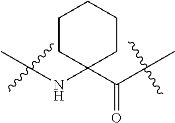 [SMILES: *NC1(C(*)=O)CCCCC1] |
| Acpx or Acvc | 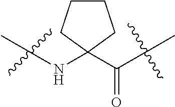 [SMILES: *NC1(C(*)=O)CCCC1] |
| AEF, Phe[4-(2-aminoethoxy)], | 4-(2-aminoethoxy)-L-phenylalanine |
| or F(4-2ae) | |
| AEF(Ac) or AcAEF | 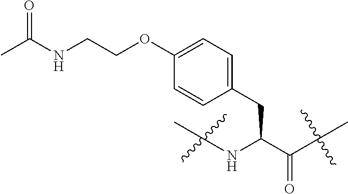 [SMILES: *N[C@@H](Cc1ccc(OCCNC(C)=O)cc1)C(*)=O] |
| AEF(AcCh) | 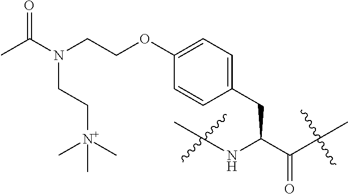 [SMILES: *N[C@@H](Cc1ccc(OCCN(CC[N+](C)(C)C)C(C)=O)cc1)C(*)=O] |
| AEF(aPEG2a) | 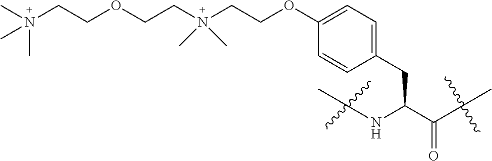 |
| AEF(BisMEP) | 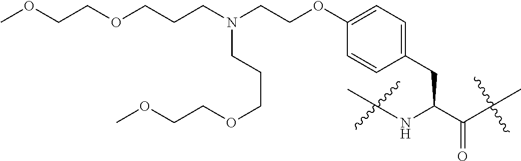 [SMILES: *N[C@@H](Cc1ccc(OCCN(CCCOCCOC)CCCOCCOC)cc1)C(*)=O] |
| AEF(BisMEPa) | 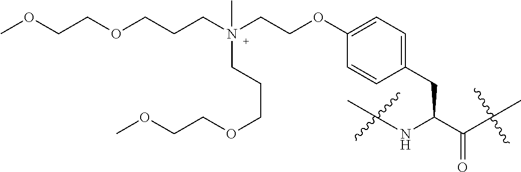 |
| AEF(BisPEG2a) | 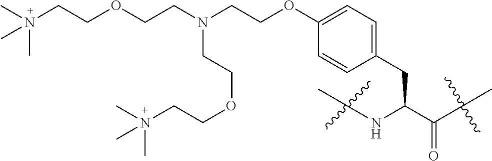 |
| AEF(BisPEG2a)(RS) | 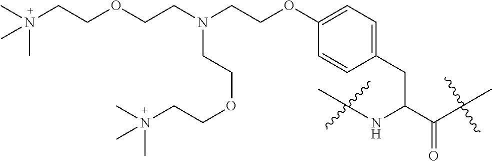 |
| AEF(BisPEG2a)(R*) | 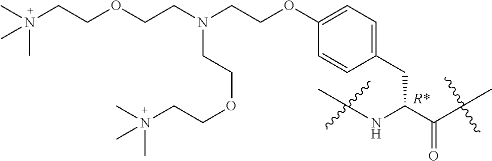 |
| AEF(BisPEG2a)(S*) | 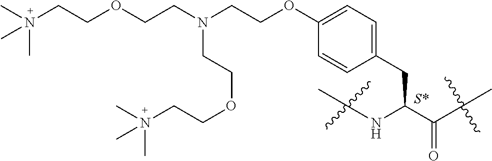 |
| AEF((Ch)cPEG3a) | 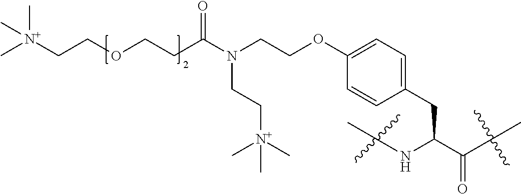 |
| AEF(G) | 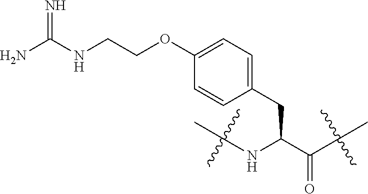 [SMILES: *N[C@@H](Cc1ccc(OCCNC(=N)N)cc1)C(*)=O] |
| AEF(Me)2 or AEF(N(Me)2) | 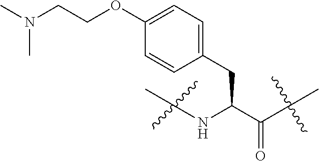 [SMILES: *N[C@@H](Cc1ccc(OCCN(C)C)cc1)C(*)=O] |
| AEF(MEP) | 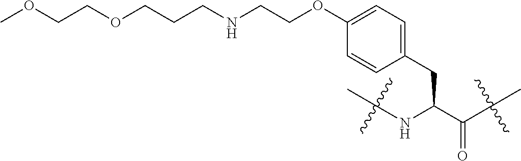 [SMILES: *N[C@@H](Cc1ccc(OCCNCCCOCCOC)cc1)C(*)=O] |
| AEF(MePrpa) | 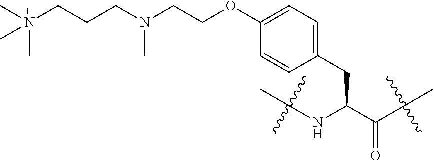 |
| AEF(NHCh) | 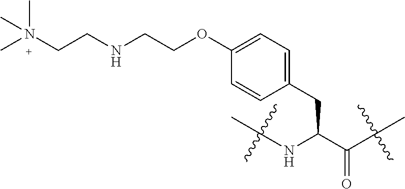 |
| AEF(NHcPEG3a) or AEF(cPEG3a) | 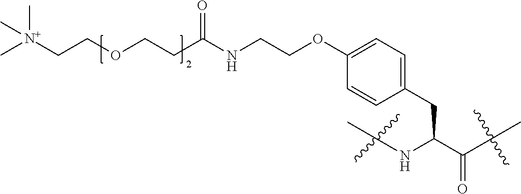 |
| AEF(NMe) | 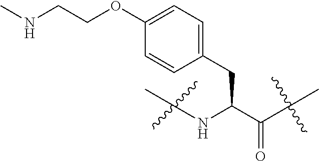 [SMILES: *N[C@@H](Cc1ccc(OCCNC)cc1)C(*)=O] |
| AEF(NMe2mPEG3) | 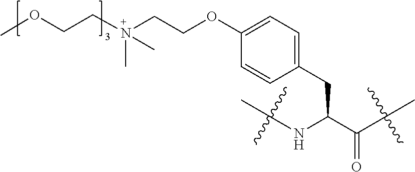 |
| AEF(NMe3) | 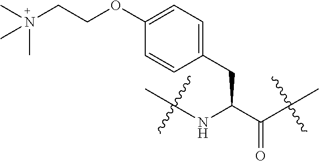 |
| AEF(NMeBismPEG3) or AEF(aBismPEG3) | 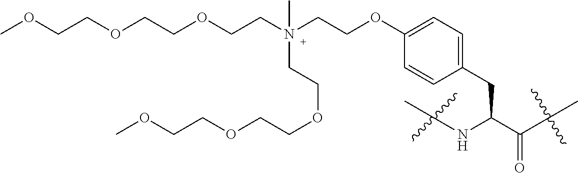 |
| AEF(NMePEG2a) | 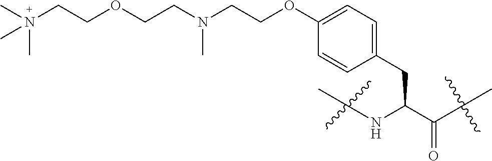 |
| AEF(NmPEG6) | 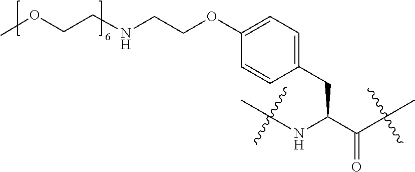 |
| AEF(NsCh) | 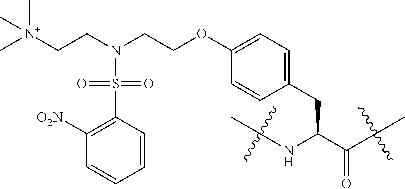 [SMILES: *N[C@@H](Cc1ccc(OCCN(CC[N+](C)(C)C)S(=O)(=O)c2ccccc2[N+](=O)[O-])cc1)C(*)=O] |
| AEF(PEG2a) | 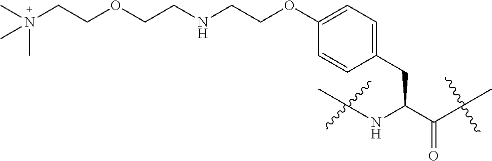 |
| AEF(SPD) | 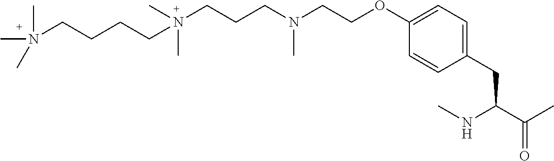 |
| APEG2F | 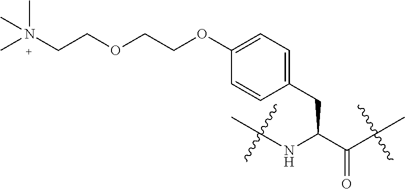 |
| APEG3F | 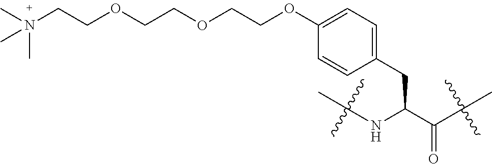 |
| Ahx | 6-aminohexanoic acid |
| AIB | 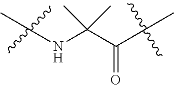 [SMILES: *NC(C)(C)C(*)=O] |
| aMeC | alpha-methyl L-cystine |
| aMeE | alpha-methyl L-glutamic acid |
| aMeLeu or aMeL | alpha-methyl L-leucine |
| aMeLys or aMeK | alpha-methyl L-lysine |
| APEG2ser | 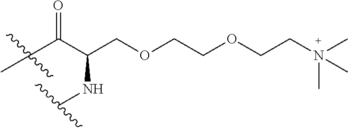 |
| APEG2Ser | 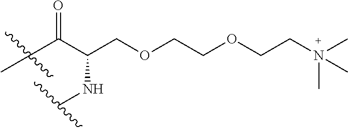 |
| APEG2Ser(RS) | 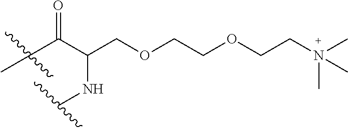 |
| APEG2Ser(S*) | 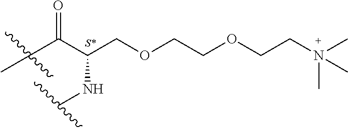 |
| APEG2Ser(R*) | 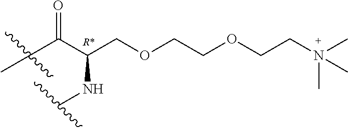 |
| APF | 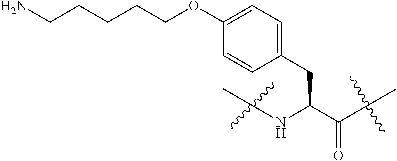 [SMILES: *N[C@@H](Cc1ccc(OCCCCCN)cc1)C(*)=O] |
| bAla | 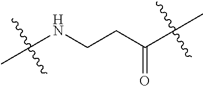 [SMILES: *NCCC(*)=O] |
| Chg | 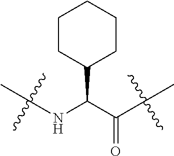 [SMILES: *N[C@H](C(*)=O)C1CCCCC1] |
| Cit | 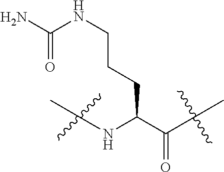 [SMILES: *N[C@@H](CCCNC(N)=O)C(*)=O] |
| Dab | L-2,4-diaminobutyric acid |
| Dab(Me)3 | 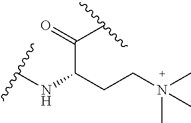 |
| Dab(NMeAc) | 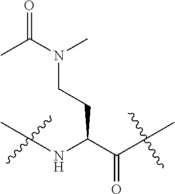 [SMILES: *N[C@@H](CCN(C)C(C)=O)C(*)=O] |
| Dab(NMecarn) | 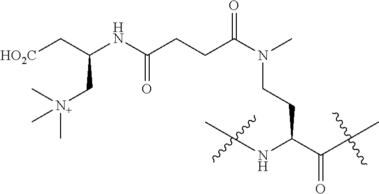 [SMILES: *N[C@@H](CCN(C)C(=O)CCC(=O)N[C@H](CC(=O)O)C[N+](C)(C)C)C(*)=O] |
| Dab(NMeCarn) | 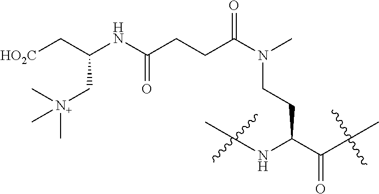 [SMILES: *N[C@@H](CCN(C)C(=O)CCC(=O)N[C@@H](CC(=O)O)C[N+](C)(C)C)C(*)=O] |
| Dab(NMeCOmPEG6) | 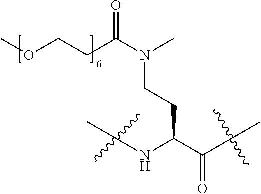 |
| Dab(NMecPEG2a) | 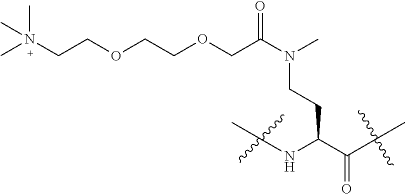 |
| Dab(NMecPEG3a) | 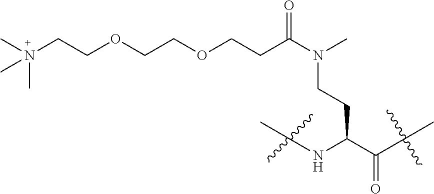 |
| Dab(NMecPEG5a) or Dab(NMecPEG5aCO)- | 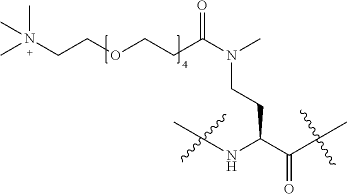 |
| Dap | L-2,3-diaminopropionic acid |
| Dap(Ac) | 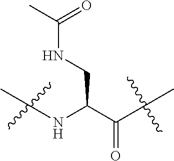 [SMILES: *N[C@@H](CNC(C)=O)C(*)=O] |
| Dap(NMeAc) | 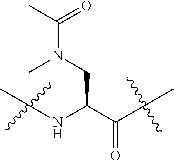 [SMILES: *N[C@@H](CN(C)C(C)=O)C(*)=O] |
| diFCpx | 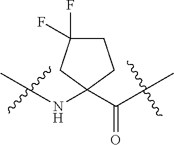 [SMILES: *NC1(C(*)=O)CCC(F)(F)C1] |
| dFPPEG3F | 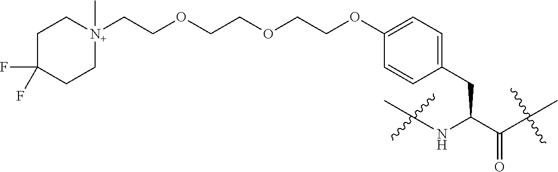 [SMILES: *N[C@@H](Cc1ccc(OCCOCCOCC[N+]2(C)CCC(F)(F)CC2)cc1)C(*)=O] |
| Diazabiclyclooctane6F | 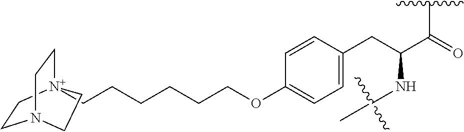 |
| DMMMF | 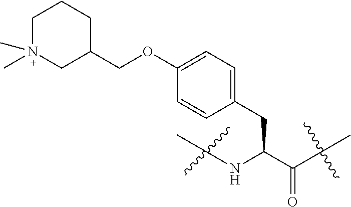 |
| DMPMF | 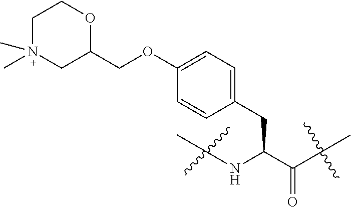 |
| DMTASF | 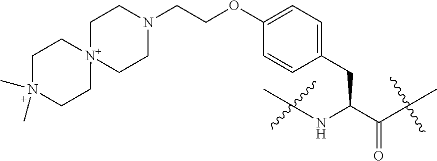 |
| e(c) | 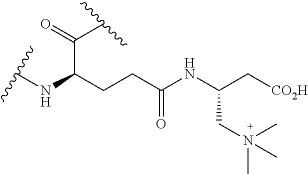 |
| e(C) | 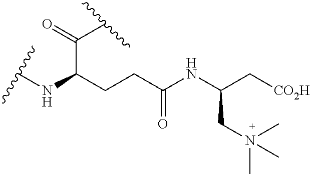 |
| E(c) | 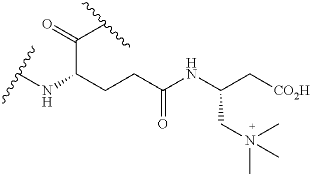 |
| E(C) | 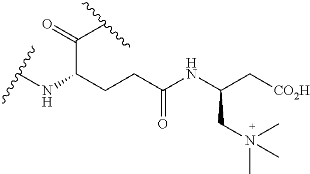 |
| F | L-phenylalanine |
| F(4G) | 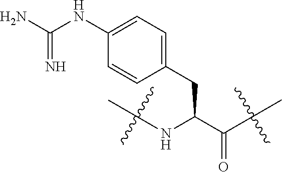 [SMILES: *N[C@@H](Cc1ccc(NC(=N)N)cc1)C(*)=O] |
| F(4N3) | 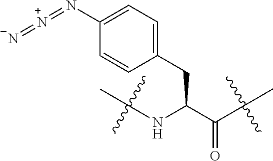 [SMILES: *N[C@@H](Cc1ccc(N=[N+]=[N-])cc1)C(*)=O] |
| F(4TzlAme2) | 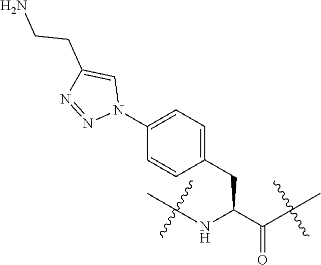 [SMILES: *N[C@@H](Cc1ccc(-n2cc(CCN)nn2)cc1)C(*)=O] |
| F(4TzlDMA4mPEG) | 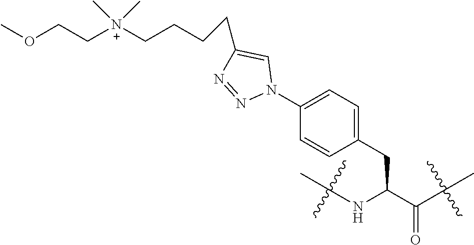 [SMILES: *N[C@@H](Cc1ccc(-n2cc(CCCC[N+](C)(C)CCOC)nn2)cc1)C(*)=O] |
| F(4TzlG2) | 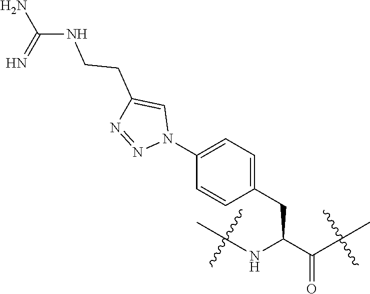 [SMILES: *N[C@@H](Cc1ccc(-n2cc(CCNC(=N)N)nn2)cc1)C(*)=O] |
| F(4TzlMME) | 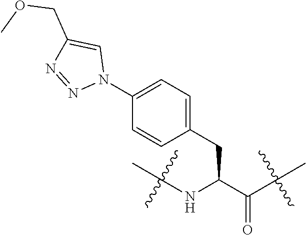 [SMILES: *N[C@@H](Cc1ccc(-n2cc(COC)nn2)cc1)C(*)=O] |
| F(4TzlMMo1) | 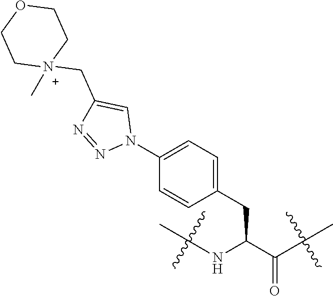 [SMILES: *N[C@@H](Cc1ccc(-n2cc(C[N+]3(C)CCOCC3)nn2)cc1)C(*)=O] |
| F(4TzlMMo3) | 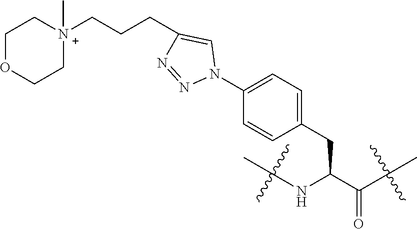 [SMILES: *N[C@@H](Cc1ccc(-n2cc(CCC[N+]3(C)CCOCC3)nn2)cc1)C(*)=O] |
| F(4TzlMMo4) | 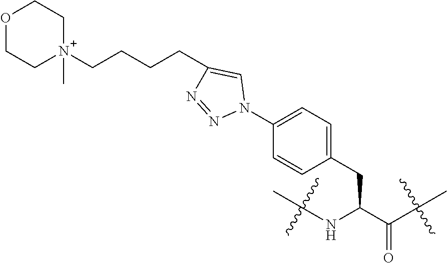 [SMILES: *N[C@@H](Cc1ccc(-n2cc(CCCC[N+]3(C)CCOCC3)nn2)cc1)C(*)=O] |
| F(4TzlMMo7) | 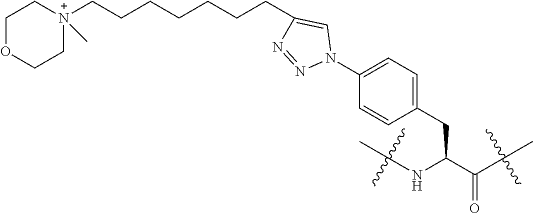 [SMILES: *N[C@@H](Cc1ccc(-n2cc(CCCCCCC[N+]3(C)CCOCC3)nn2)cc1)C(*)=O] |
| F(4TzlTMA1) | 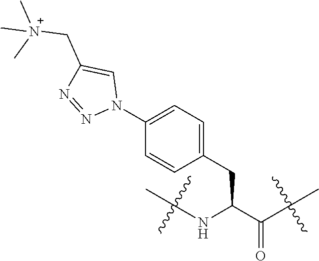 [SMILES: *N[C@@H](Cc1ccc(-n2cc(C[N+](C)(C)C)nn2)cc1)C(*)=O] |
| F(4TzlTMA2) | 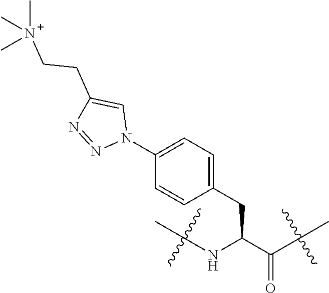 [SMILES: *N[C@@H](Cc1ccc(-n2cc(CC[N+](C)(C)C)nn2)cc1)C(*)=O] |
| F(4TzlTMA3) | 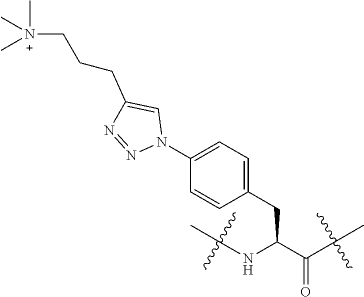 [SMILES: *N[C@@H](Cc1ccc(-n2cc(CCC[N+](C)(C)C)nn2)cc1)C(*)=O] |
| F(4TzlTMA4) | 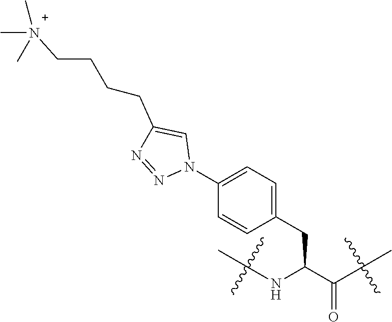 [SMILES: *N[C@@H](Cc1ccc(-n2cc(CCCC[N+](C)(C)C)nn2)cc1)C(*)=O] |
| F(4TzlTMA5) | 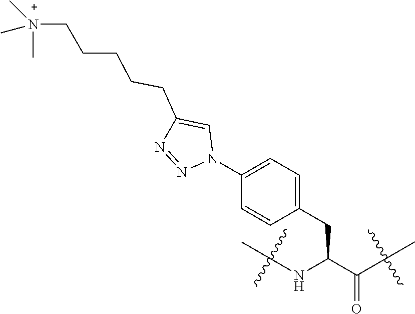 [SMILES: *N[C@@H](Cc1ccc(-n2cc(CCCCC[N+](C)(C)C)nn2)cc1)C(*)=O] |
| F(4TzlTMA7) | 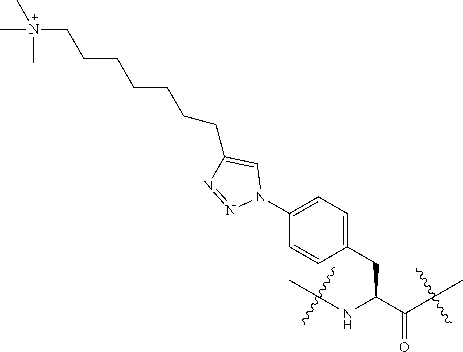 [SMILES: *N[C@@H](Cc1ccc(-n2cc(CCCCCCC[N+](C)(C)C)nn2)cc1)C(*)=O] |
| Fmoc-2Nal-OH | (S)-2-((((9H-fluoren-9- |
| | yl)methoxy)carbonyl)amino)-3-(naphthalen-2- |
| | yl)propanoic acid |
| Fmoc-3Pya-OH | (S)-2-((((9H-fluol)methoxy)carbonyl)amino)-3- |
| | (pyridin-3-yl)propanoic acid |
| Fmoc-7MeW-OH | (S)-2-((((9H-fluoren-9- |
| | yl)methoxy)carbonyl)amino)-3-(7-methyl-1H- |
| | indol-3-yl)propanoic acid |
| Fmoc-AEF(Boc)-OH | (S)-2-((((9H-fluoren-9- |
| | yl)methoxy)carbonyl)amino)-3-(4-(2-((tert- |
| | butoxycarbonyl)amino)ethoxy)phenyl)propanoic |
| | acid |
| Fmoc-AEF-OH | (S)-2-((((9H-fluoren-9- |
| | yl)methoxy)carbonyl)amino)-3-(4-(2- |
| | aminoethoxy)phenyl)propanoic acid |
| Fmoc-arg-OH or Fmoc-r-OH | (((9H-fluoren-9-yl)methoxy)carbonyl)-D-arginine |
| Fmoc-arg(Pbf)-OH or Fmoc- | N2-(((9H-fluoren-9-yl)methoxy)carbonyl)-Nw- |
| r(Pbf)-OH | ((2,2,4,6,7-pentamethyl-2,3-dihydrobenzofuran-5- |
| | yl)sulfonyl)-D-arginine |
| Fmoc-Asn(Trt)-OH or Fmoc- | N2-(((9H-fluoren-9-yl)methoxy)carbonyl)-N4- |
| N(Trt)-OH | trityl-L-asparagine |
| Fmoc-Asn-OH or | (((9H-fluoren-9-yl)methoxy)carbonyl)-L- |
| Fmoc-N-OH | asparagine |
| Fmoc-Dap(Dde)-OH | (S)-2-((((9H-fluoren-9- |
| | yl)methoxy)carbonyl)amino)-3-((1-(4,4-dimethyl- |
| | 2,6-dioxocyclohexylidene)ethyl)amino)propanoic |
| | acid |
| Fmoc-Dde-Lys(Fmoc)-OH | N6-(((9H-fluoren-9-yl)methoxy)carbonyl)-N2-(1- |
| | (4,4-dimethyl-2,6-dioxocyclohexylidene)ethyl)-L- |
| | lysine |
| Fmoc-gE-OtBu | (S)-4-((((9H-fluoren-9- |
| | yl)methoxy)carbonyl)amino)-5-(tert-butoxy)-5- |
| | oxopentanoic acid |
| Fmoc-gE-OAll | (S)-4-((((9H-fluoren-9- |
| | yl)methoxy)carbonyl)amino)-5-(allyloxy)-5- |
| | oxopentanoic acid |
| Fmoc-aMeGlu(tBu) or Fmoc- | (S)-2-((((9H-fluoren-9- |
| aMeE(tBu) | yl)methoxy)carbonyl)amino)-5-(tert-butoxy)-2- |
| | methyl-5-oxopentanoic acid |
| Fmoc-Glu-OH or Fmoc-E-OH | (S)-2-((((9H-fluoren-9- |
| | yl)methoxy)carbonyl)amino)-5-(tert-butoxy)-5- |
| | oxopentanoic acid |
| Fmoc-GolA(tBu)-OH | 2-((((9H-fluoren-9-yl)methoxy)carbonyl)amino)- |
| | 3-(tert-butoxy)-2-(tert-butoxymethyl)propanoic |
| | acid |
| Fmoc-Lys(Ac)-OH or Fmoc- | N2-(((9H-fluoren-9-yl)methoxy)carbonyl)-N6- |
| K(Ac)-OH | acetyl-L-lysine |
| Fmoc-Lys(Dde)-OH or Fmoc- | N2-(Fmoc)-N6-(1-(4,4-dimethyl-2,6- |
| K(Dde)-OH | dioxocyclohexylidene)ethyl)-L-lysine |
| Fmoc-Lys(NMeAc)-OH or | N2-(((9H-fluoren-9-yl)methoxy)carbonyl)-n6- |
| Fmoc-K(NMeAc)-OH | acetyl-N6-methyl-L-lysine |
| Fmoc-NMeLys(Dde)-OH or | (9H-fluoren-9-yl)methyl (R)-(1-amino-6-((1-(4,4- |
| Fmoc-NMeK(Dde)-OH | dimethyl-2,6-dioxocyclohexylidene)ethyl)amino)-1- |
| | oxohexan-2-yl)(methyl)carbamate |
| Fmoc-Pen(Trt)-OH | (R)-2-((((9H-fluoren-9- |
| | yl)methoxy)carbonyl)amino)-3-methyl-3- |
| | (tritylthio)butanoic acid |
| Fmoc-Pro-OH or | Fmoc-L-proline |
| Fmoc-P-OH | |
| Fmoc-pro-OH or | Fmoc-D-proline |
| Fmoc-p-OH | |
| Fmoc-Sar-OH or | N-(((9H-fluoren-9-yl)methoxy)carbonyl)-N- |
| Fmoc-Sarc-OH | methylglycine |
| Fmoc-THP-OH | 4-((((9H-fluoren-9- |
| | yl)methoxy)carbonyl)amino)tetrahydro-2H-pyran- |
| | 4-carboxylic acid |
| Fmoc-Thr(tBu)-OH or | N-(((9H-fluoren-9-yl)methoxy)carbonyl)-O-(tert- |
| Fmoc-T(tBu)-OH | butyl)-L-threonine |
| Gab | 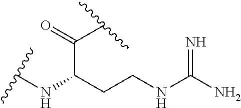 [SMILES: *N[C@@H](CCNC(=N)N)C(*)=O] |
| gE-OtBu | (S)-4-(amino)-5-(tert-butoxy)-5-oxopentanoic |
| | acid |
| gE | 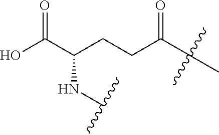 [SMILES: *N[C@@H](CCC(*)=O)C(=O)O] |
| Glu or E | L-glutamic acid |
| GolA(tBu) | 2-(amino)-3-(tert-butoxy)-2-(tert- |
| | butoxymethyl)propanoic acid |
| GolA | 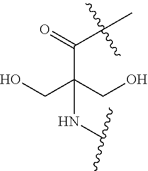 [SMILES: *NC(CO)(CO)C(*)=O] |
| GolB(tBu) | 1,3-di-tert-butoxypropan-2-amine |
| GolB | 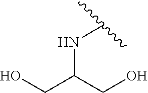 [SMILES: *NC(CO)CO] |
| gPEG2Ser | 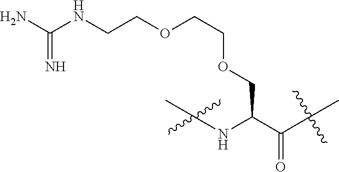 [SMILES: *N[C@@H](COCCOCCNC(=N)N)C(*)=O] |
| GPEG3F | 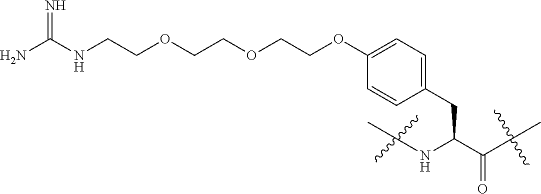 [SMILES: *N[C@@H](Cc1ccc(OCCOCCOCCNC(=N)N)cc1)C(*)=O] |
| hFTMAPF | 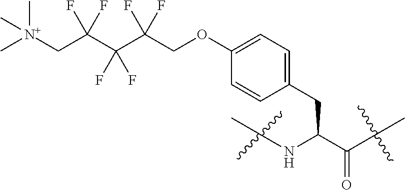 [SMILES: *N[C@@H](Cc1ccc(OCC(F)(F)C(F)(F)C(F)(F)C[N+](C)(C)C)cc1)C(*)=O] |
| hk(Me)3 | 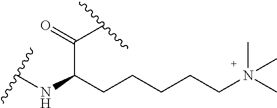 |
| K(4cpg) | 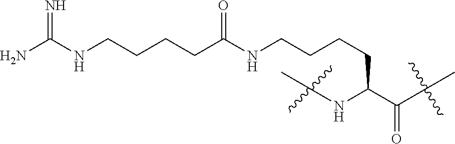 [SMILES: *N[C@@H](CCCCNC(=O)CCCCNC(=N)N)C(*)=O] |
| K(5cpa) | 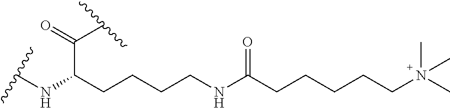 |
| k(5cpa) | 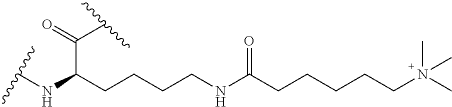 |
| K(Ac) | 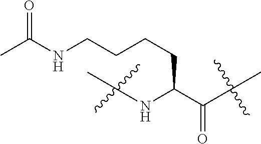 [SMILES: *N[C@@H](CCCCNC(C)=O)C(*)=O] |
| K(cPEG3a) or K(cPEG3aCO) | 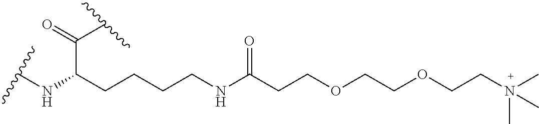 |
| k(cPEG3a) | 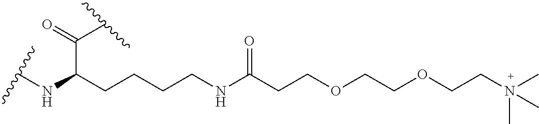 |
| K(d) | 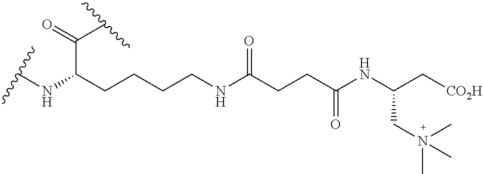 |
| K(D) | 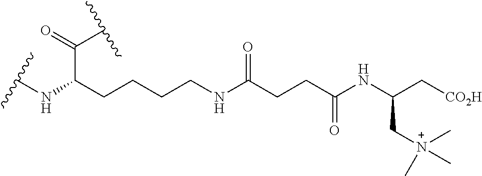 [SMILES: *N[C@@H](CCCCNC(=O)CCC(=O)N[C@@H](CC(=O)O)C[N+](C)(C)C)C(*)=O] |
| k(d) | 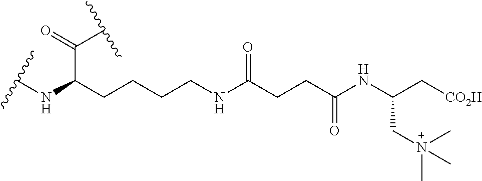 [SMILES: *N[C@H](CCCCNC(=O)CCC(=O)N[C@H](CC(=O)O)C[N+](C)(C)C)C(*)=O] |
| k(D) | 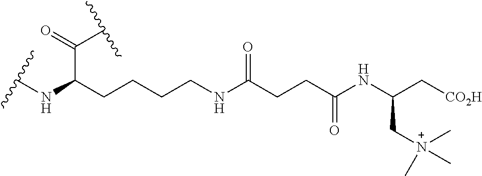 [SMILES: *N[C@H](CCCCNC(=O)CCC(=O)N[C@@H](CC(=O)O)C[N+](C)(C)C)C(*)=O] |
| K(dPEG6Ac) | 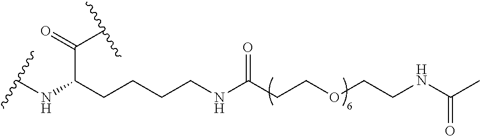 |
| k(dPEG6Ac) | 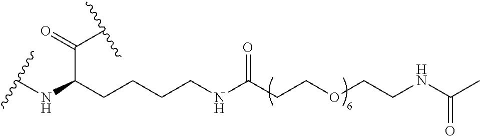 |
| K(dPEG9Ac) | 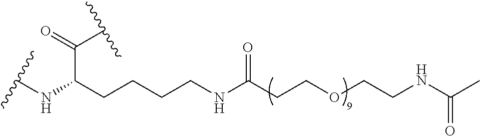 |
| k(dPEG9Ac) | 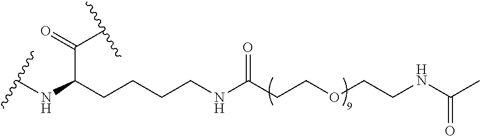 |
| K(dPEG12Ac) | 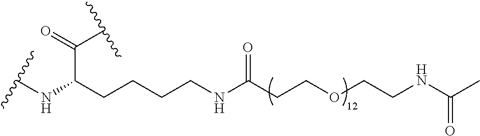 |
| k(dPEG12Ac) | 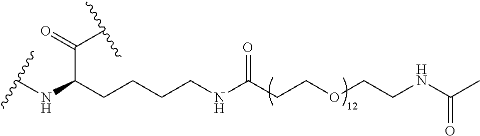 |
| K(IPB) | 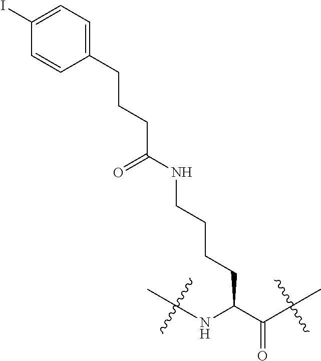 [SMILES: *N[C@@H](CCCCNC(=O)CCCc1ccc(I)cc1)C(*)=O] |
| K(DFN) | 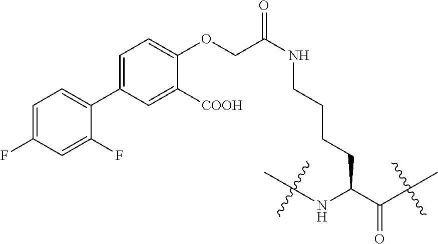 [SMILES: *N[C@@H](CCCCNC(=O)COc1ccc(-c2ccc(F)cc2F)cc1C(=O)O)C(*)=O] |
| k(Me)3 | 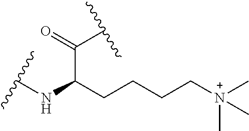 [SMILES: *N[C@H](CCCC[N+](C)(C)C)C(*)=O] |
| K(Me)3 | 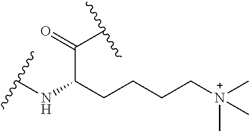 [SMILES: *N[C@@H](CCCC[N+](C)(C)C)C(*)=O] |
| K(NMeAc) or K(NMeAC) | 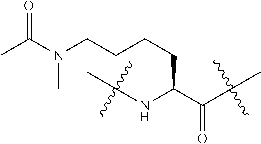 [SMILES: *N[C@@H](CCCCN(C)C(C)=O)C(*)=O] |
| K(NMeCOmPEG6) | 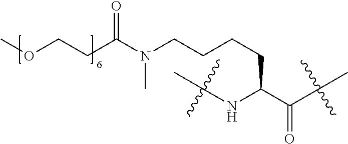 |
| K(NMeCOPEG4N + Me3) or K(NMeCOPEG5a) or K(NMecPEG5a) or K(NMePEG5a) | 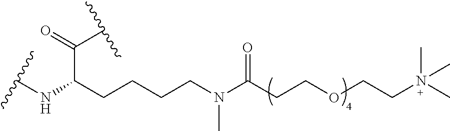 |
| K(NMePEG3a) | 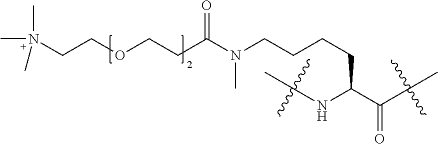 |
| K(NmPEG6Ac) | 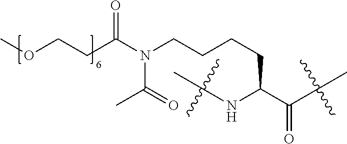 |
| K(PEG2PEG2gEC12) | 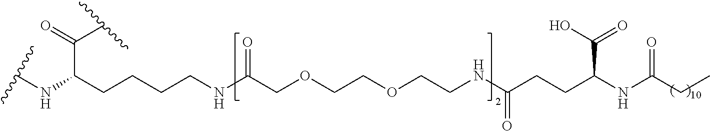 |
| k(PEG2PEG2gEC12) | 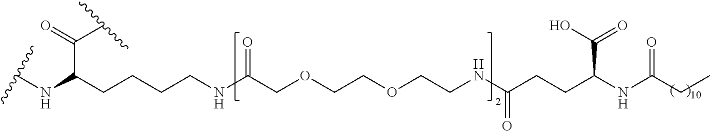 |
| k(PEG2PEG2gE(c)C12 | 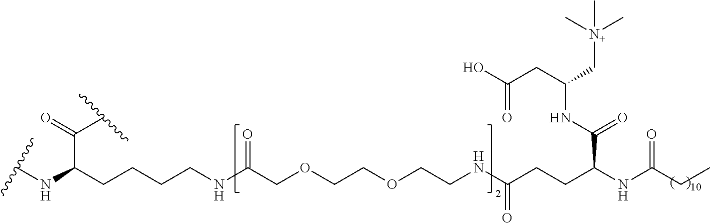 |
| K(PEG2PEG2gEC14) | 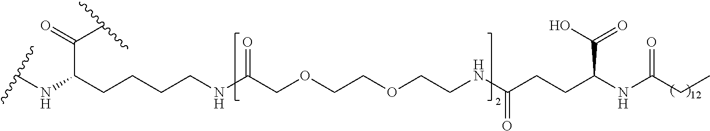 |
| k(PEG2PEG2gEC14) | 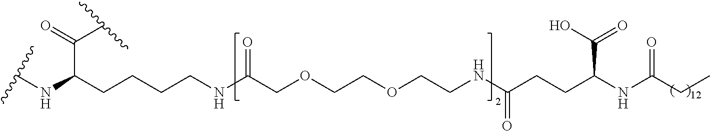 |
| k(PEG2PEG2PEG2 PEG2gEC12) | 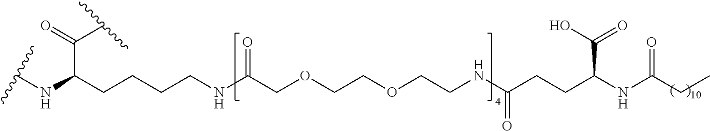 |
| K(PEG2PEG2PEG2gEC12) | 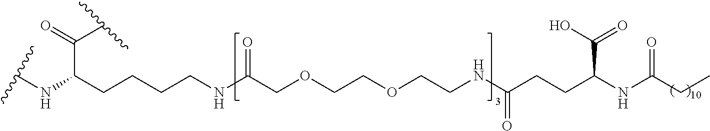 |
| k(PEG2PEG6gEC12) | 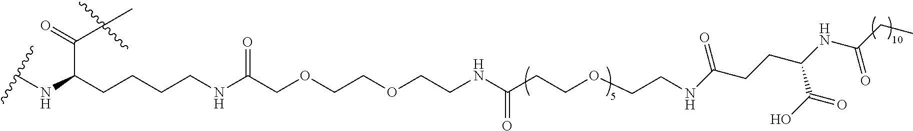 |
| L | L-leucine |
| dL | D-leucine |
| Lys or K | L-lysine |
| dK | D-lysine |
| Lys(Ac) or K(Ac) | N-ε-acetyl-L-lysine |
| Me3Pya | 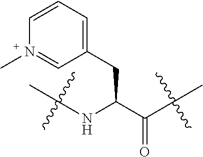 |
| MMoEF | 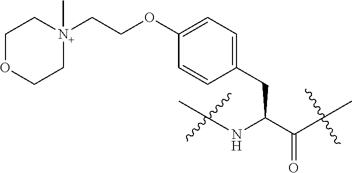 |
| MMoPF | 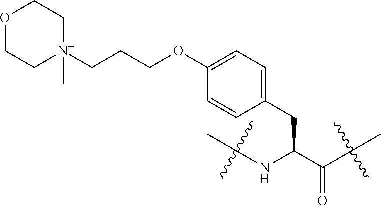 [SMILES: *N[C@@H](Cc1ccc(OCCC[N+]2(C)CCOCC2)cc1)C(*)=O] |
| MMPEG3F | 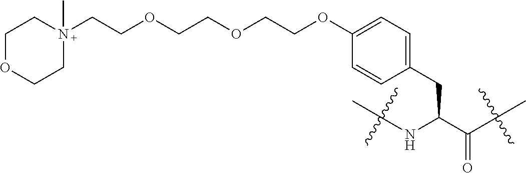 |
| morfTMA4F | 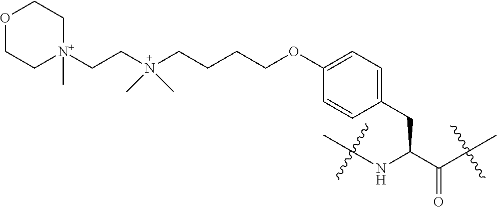 |
| mPEG2TMA2F or mPEGTMA2F | 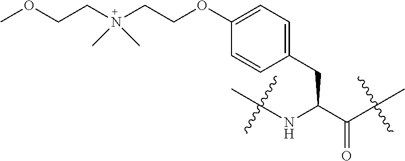 |
| mPEG2TMA4F or mPEGTMA4F | 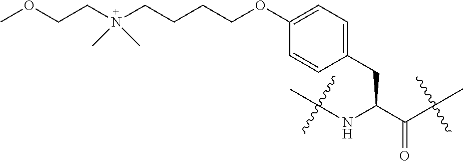 |
| mPEG3TMA4F | 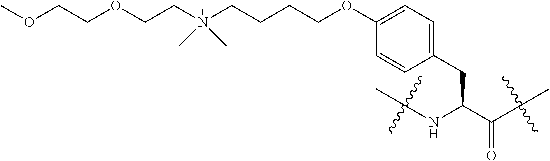 |
| MPzPEG3F | 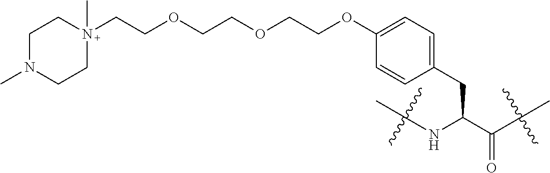 |
| MTASF | 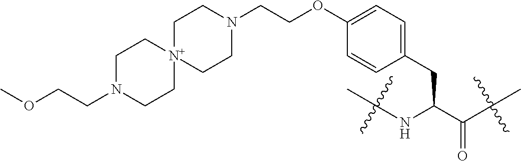 [SMILES: *N[C@@H](Cc1ccc(OCCN2CC[N+]3(CCN(CCOC)CC3)CC2)cc1)C(*)=O] |
| N | L-asparagine |
| Nle | 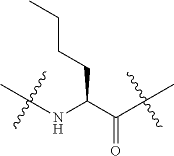 [SMILES: *N[C@@H](CCCC)C(*)=O] |
| NMe3Pya | 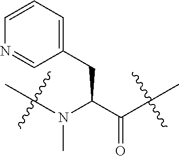 [SMILES: *C(=O)[C@H](Cc1cccnc1)N(*)C] |
| NMebAla | 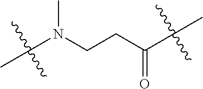 [SMILES: *C(=O)CCN(*)C] |
| NMeDTyr | 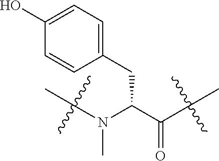 [SMILES: *C(=O)[C@@H](Cc1ccc(O)cc1)N(*)C] |
| N(N(Me)) or N(NMe) | 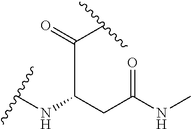 [SMILES: *N[C@@H](CC(=O)NC)C(*)=O] |
| N(N(Me)2) | 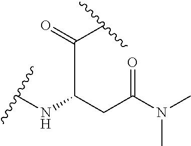 [SMILES: *N[C@@H](CC(=O)N(C)C)C(*)=O] |
| N-MeLys, NMeLys, or | N-methyl-lysine |
| NMeK | |
| NMeK(PEG2pEG2C12) | 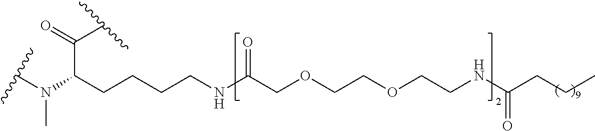 |
| NMeK(PEG2pEG2C14) | 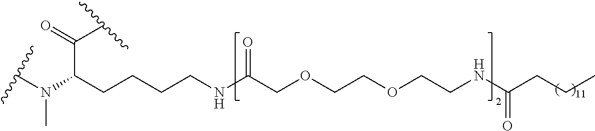 |
| NMeK(PEG2PEG2gEC12) | 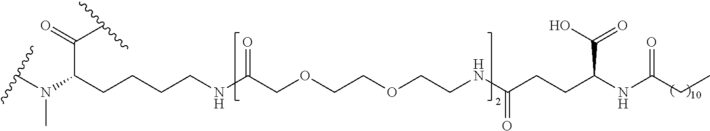 |
| NMeK(PEG2PEg2gEC14) | 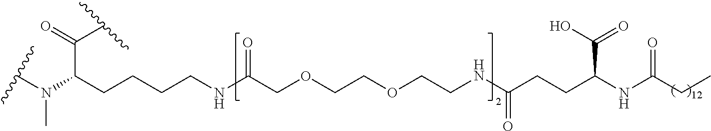 |
| NMeK(PEG2PEG2K (Peg2Peg2gEC12)2) | 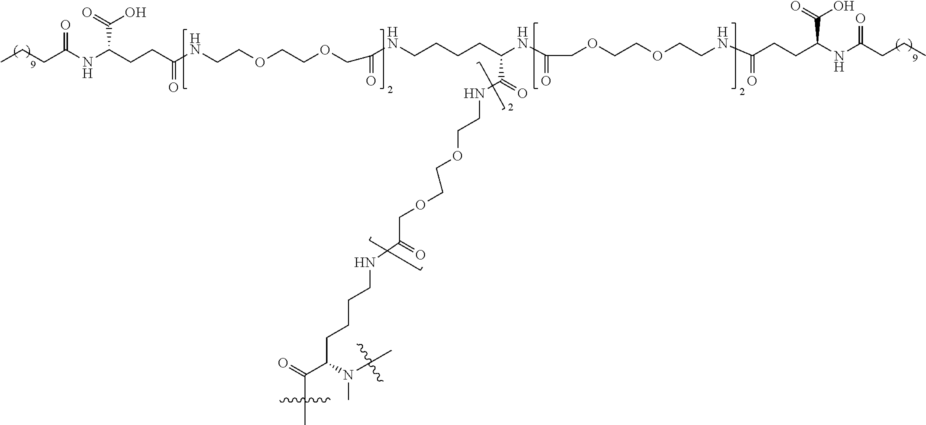 |
| NMeK(PEG2PEG2K (PEG2PEG2 PEG2PEG2gEC12)2) | 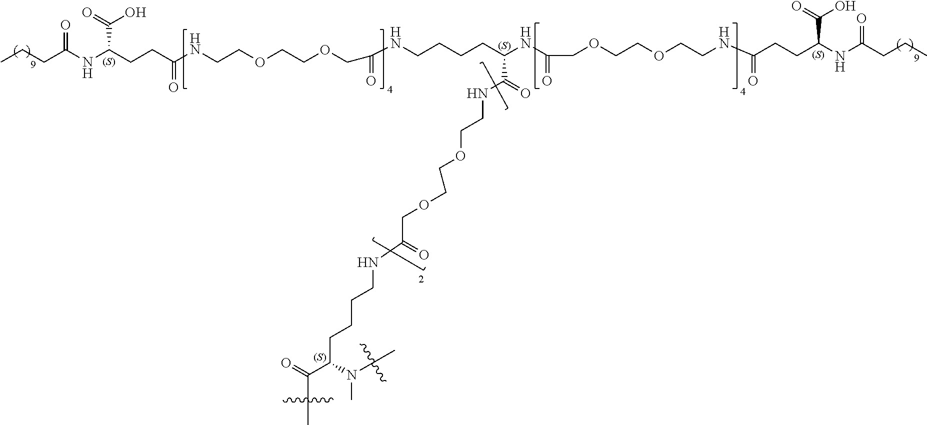 |
| NMeK(PEG2 PEG2PEg2gEC12) | 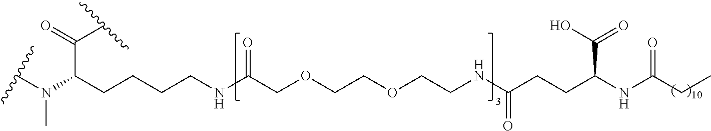 |
| NMeK(PEG2PEG2 PEG2PEg2gEC12) | 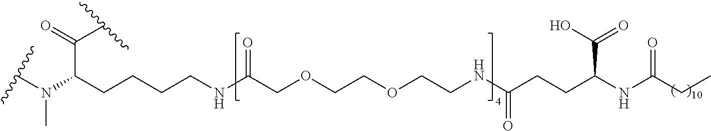 |
| NMeK(PEG2PEg6gEC12) | 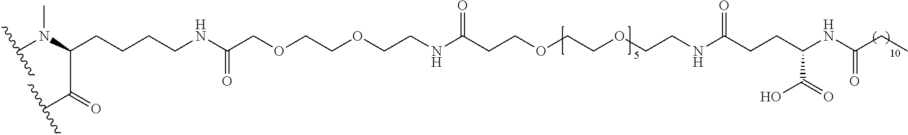 |
| NMeK (PEG2PEg6gEC14) | 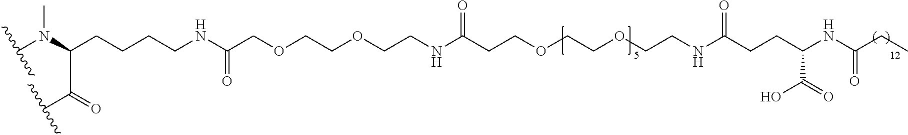 |
| NMeK (SP6PEG2pEG2C12) | 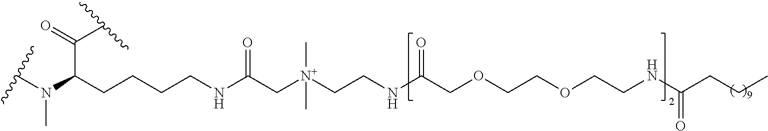 |
| NMeK (SP6PEG2PEg2gEC12) | 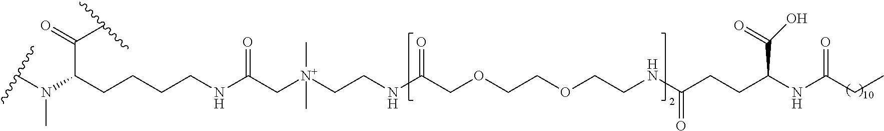 |
| NPyEF | 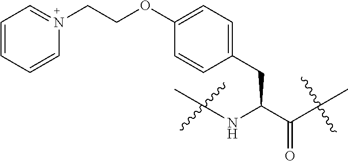 |
| NPyPEG3F | 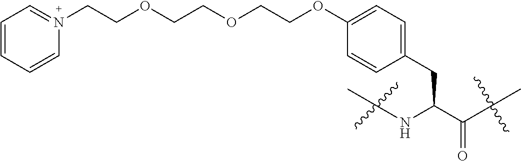 |
| orn | D-ornithine |
| Paf | 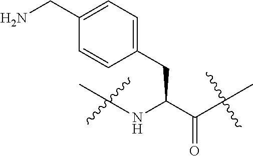 [SMILES: *N[C@@H](Cc1ccc(CN)cc1)C(*)=O] |
| Paf(Ac) | 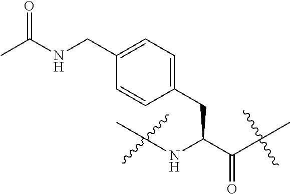 [SMILES: *N[C@@H](Cc1ccc(CNC(C)=O)cc1)C(*)=O] |
| Pen | L-penicillamine |
| Phe(4-CONH2) or 4AmF or | (S)-2-amino-3-(4-carbamoylphenyl)propanoic acid |
| 4AmPhe | |
| Phe(4-OMe) or F(4-OMe) or | 4-methoxy-L-phenylalanine |
| F(4Ome) or 4OMeF | |
| Pip(nMe) | 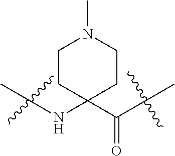 [SMILES: *NC1(C(*)=O)CCN(C)CC1] |
| Pip(NMe2) | 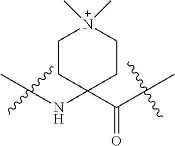 [SMILES: *NC1(C(*)=O)CC[N+](C)(C)CC1] |
| PiperazinequatF | 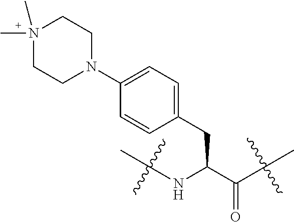 |
| Q | L-glutamine |
| Q(N(Me)2) | 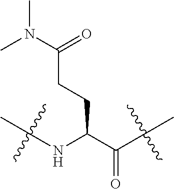 [SMILES: *N[C@@H](CCC(=O)N(C)C)C(*)=O] |
| Q(NHtBu) | 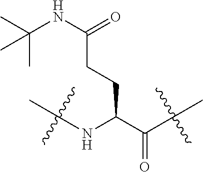 [SMILES: *N[C@@H](CCC(=O)NC(C)(C)C)C(*)=O] |
| r or dR | D-arginine |
| Sar or Sarc | sarcosine or N-methylglycine |
| SP6 | 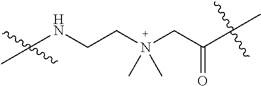 |
| TBAPEG3F | 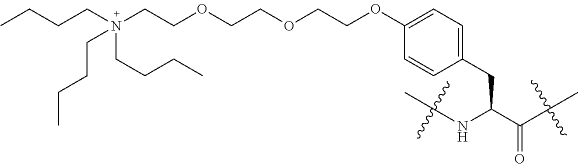 |
| Tetrazole | 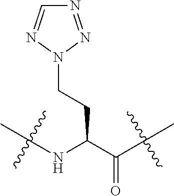 [SMILES: *N[C@@H](CCn1ncnn1)C(*)=O] |
| Tetrazole(NMe) | 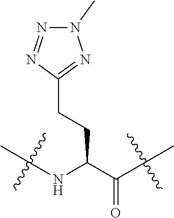 [SMILES: *N[C@@H](CCc1nnn(C)n1)C(*)=O] |
| Thr or T | L-threonine |
| TMA3F | 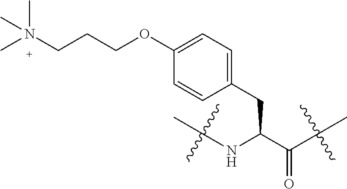 |
| TMA4F | 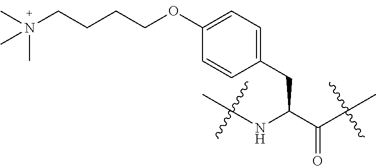 |
| TMA6F | 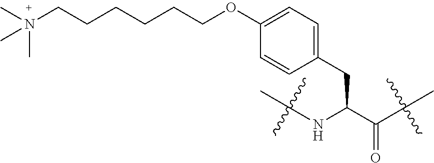 |
| TMA8F | 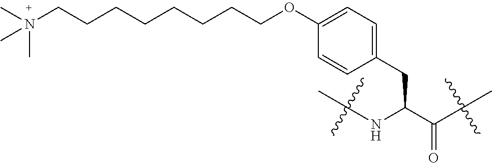 |
| TMAPF | 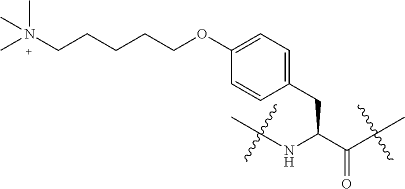 |
| Trx | (1R,4R)-4-(aminomethyl)cyclohexane-1- |
| | carboxylic acid |
| Tzl(Ch) | 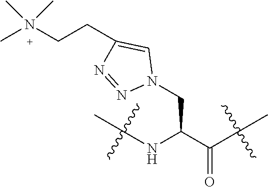 |
| TzlChmPEG | 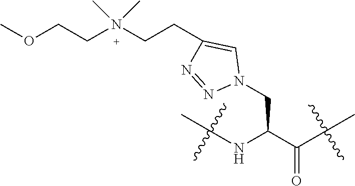 |
| TzlChmPEG3 | 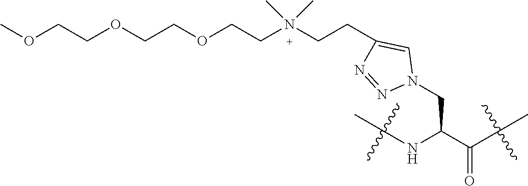 |
| W | L-tryptophan |
| Y | L-tyrosine |
| Y(C9OH) | 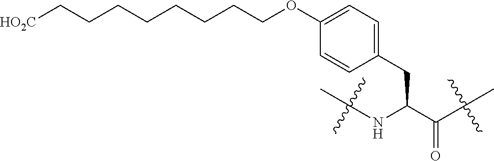 [SMILES: *N[C@@H](Cc1ccc(OCCCCCCCCC(=O)O)cc1)C(*)=O] |
| Y(CHF2) or Y(CF2H) | 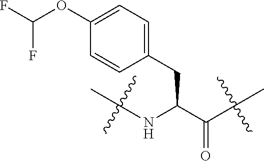 [SMILES: *N[C@@H](Cc1ccc(OC(F)F)cc1)C(*)=O] |
| Y(OEOXiMECh) | 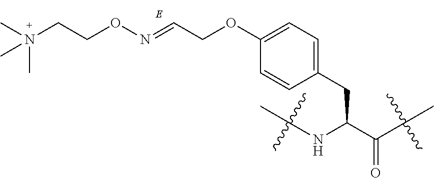 |
| Y(OtzlCh) | 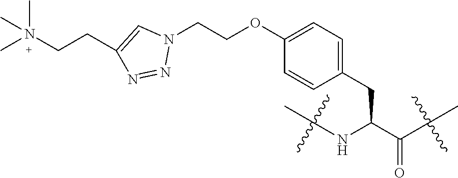 |
| Y(OTzlchC16) | 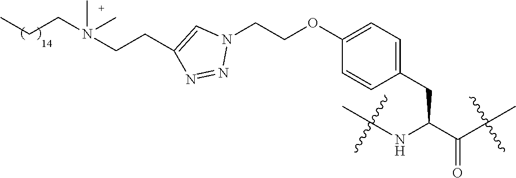 |
| Y(OTzlChC8) | 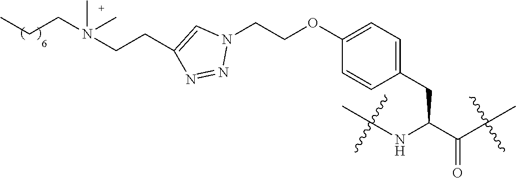 |
| Y(OTzlChmPEG) | 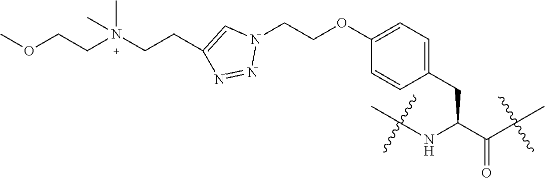 |
| Y(OTzlChmPEG3) | 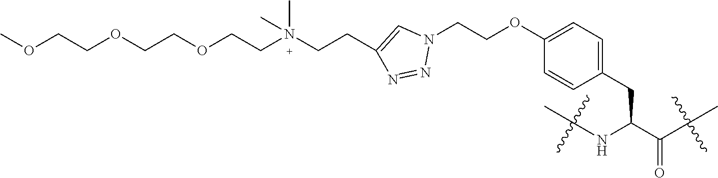 |
| Y(OTzlPEG3a) | 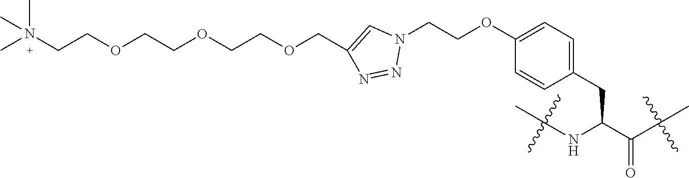 |
| Y(OTzlPEG4a) | 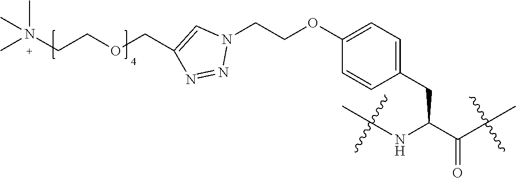 |
| Y(OTzlTMA4) | 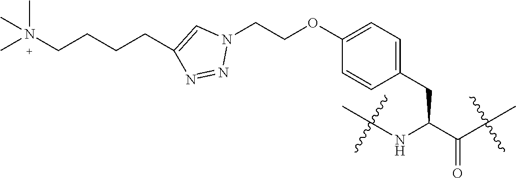 |
| Y(OZOXIMECh) | 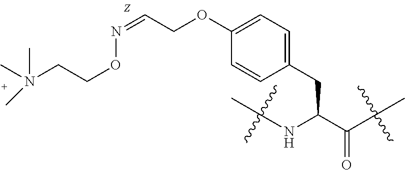 |
| YC8CO(NHPEG3a) | 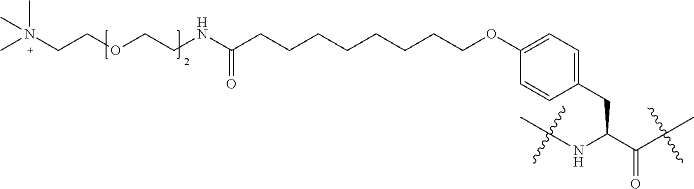 |
| YC8COPip | 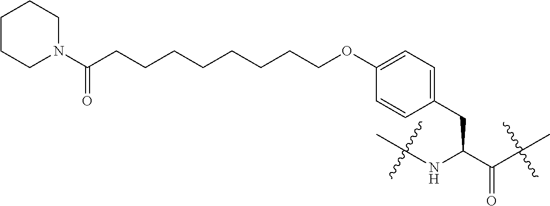 [SMILES: *N[C@@H](Cc1ccc(OCCCCCCCCC(=O)N2CCCCC2)cc1)C(*)=O] |
| Y(OTzlC1aC8) | 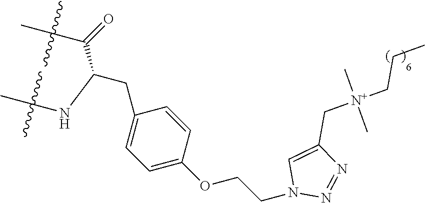 |

TABLE 2B
Abbreviations for Substituents, Reagents, and Solvents

| Abbreviation | Definition |
|---|---|
| Ac | acetyl |
| can | acetonitrile |
| Boc | tert-butoxy-carbonyl |
| Boc-mXOH | 10-(3-(tert-butoxycarbonyl)phenoxy)decanoic acid |
| Boc-pXOH | 10-(4-(tert-butoxycarbonyl)phenoxy)decanoic acid |
| tBu | tert-butyl |
| CONH2 or | carboxamide |
| CONH₂ | |
| COOH | carboxylic acid |
| DCM | dichloromethane |
| Dde | N-(1-(4,4-dimethyl-2,6-dioxocyclohexylidene)ethyl |
| DIC | N,N′-diisopropylcarbodiimide |
| DIPEA | diisopropylethylamine |
| DMAP | N,N-dimethylaminopyridine |
| DMF | N,N-dimethylformamide |
| DODT | 3,6-dioxa-1,8-octanedithiol |
| Et₂O | diethylether |
| FMOC or | ((9H-fluoren-9-yl)methoxy)carbonyl |
| Fmoc | |
| HOAT or | 1-hydroxy-7-azabenzotriazole |
| HOAt | |
| HPLC | high performance liquid chromatography |
| LCMS | liquid chromatography mass spectrometry |
| MeOH | methanol |
| Me | methyl |
| MBHA | 4-methylbenzhydrylamine |
| MPA | mobile phase A |
| MPB | mobile phase B |
| MS | mass spectrometry |
| MTBE | methyl tert-butyl ether |
| MW | microwave |
| NMP | N-methylpyrrolidone |
| Oxyma | ethyl cyano hydroxyiminoacetate |
| Pbf | 2,2,4,6,7-pentamethyldihydrobenzofuran-5-sulfonyl |
| RP-HPLC | reverse phase high performance liquid chromatography |
| TFA | trifluoroacetic acid |
| TIPS | triisopropylsilane |
| Trt | triphenylmethyl |
| UPLC-MS | ultra high performance liquid chromatography - |
| | mass spectrometry |

TABLE 2C
Abbreviations for Substituents

| Abbreviation | Structure |
|---|---|
| 4cpgCO | 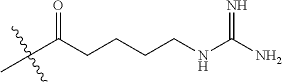 [SMILES: *C(=O)CCCCNC(=N)N] |
| 5cpaCO | —CO(CH₂)₅N(CH₃)₃⁺ |
| AcdPEG6CO | —CO(CH₂CH₂O)₆CH₂CH₂NHC(O)CH₃ |
| AcdPEG9CO | —CO(CH₂CH₂O)₉CH₂CH₂NHC(O)CH₃ |
| AcdPEG12CO | —CO(CH₂CH₂O)₁₂CH₂CH₂NHC(O)CH₃ |
| C12gEPEG2PEG2CO | 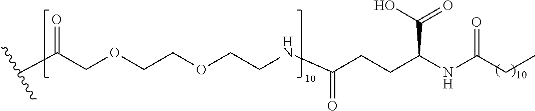 |
| C14gEPEG2PEG2CO | 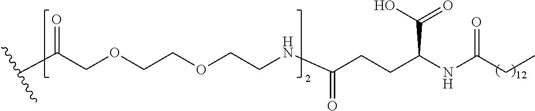 |
| CF3CO, or F3CO | 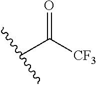 [SMILES: *C(=O)C(F)(F)F] |
| CF3propylamide | 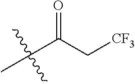 [SMILES: *C(=O)CC(F)(F)F] |
| CO(DiFPip) | 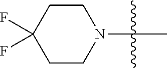 [SMILES: *N1CCC(F)(F)CC1] |
| CO(mPEG8) | 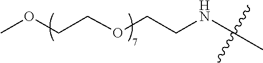 |
| CONMe2 or CON(Me)2 | 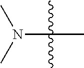 [SMILES: *N(C)C] |
| CON(MePEG2) | 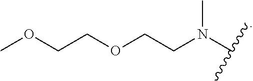 [SMILES: *N(C)CCOCCOC] |
| CONH(mPEG2) or CON(mPEG2) | 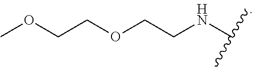 [SMILES: *NCCOCCOC] |
| CON(NMePip) | 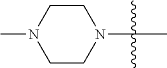 [SMILES: *N1CCN(C)CC1] |
| CONH(PEG5a) | 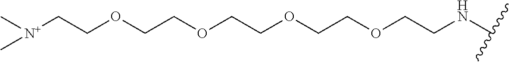 [SMILES: *NCCOCCOCCOCCOCC[NH+](C)C] |
| CONHPEG3a or CONH(PEG3a) | 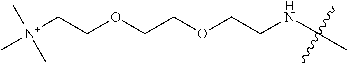 [SMILES: *NCCOCCOCC[N+](C)(C)C] |
| CO(Morph) | 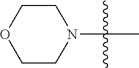 [SMILES: *N1CCOCC1] |
| CO(OAZBO) | 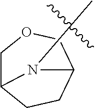 [SMILES: *N1C2CCC1COC2] |
| CO(TFMOHPip) | 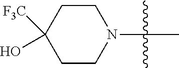 [SMILES: *N1CCC(O)(C(F)(F)F)CC1] |
| cPEG3AmCO | 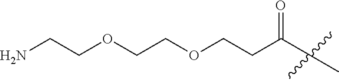 [SMILES: *C(=O)CCOCCOCCN] |
| cPEG2gCO | 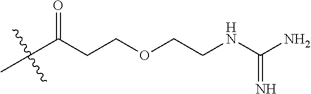 [SMILES: *C(=O)CCOCCNC(=N)N] |
| cPEG3aCO | 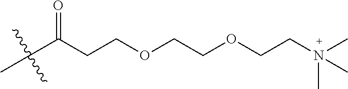 |
| cPEG3gCO | 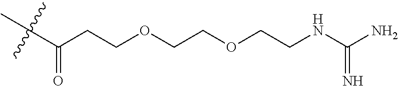 [SMILES: *C(=O)CCOCCOCCNC(=N)N] |
| cPEG5aCO | 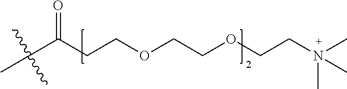 |
| DFNCO | 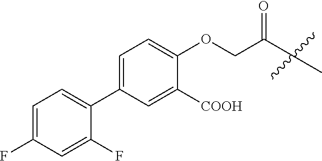 [SMILES: *C(=O)COc1ccc(-c2ccc(F)cc2F)cc1C(=O)O] |
| DFNPEG2PEG2CO | 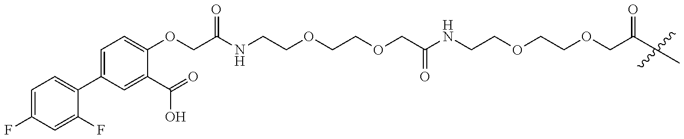 [SMILES: *C(=O)COCCOCCNC(=O)COCCOCCNC(=O)COc1ccc(-c2ccc(F)cc2F)cc1C(=O)O] |
| EtCO | 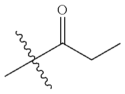 [SMILES: *C(=O)CC] |
| (d)gEPEG2PEG2CO | 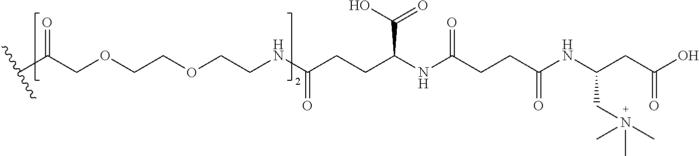 |
| IPBCO | 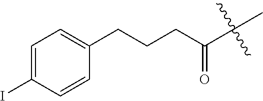 [SMILES: *C(=O)CCCc1ccc(I)cc1] |
| IPBPEG2PEG2CO | 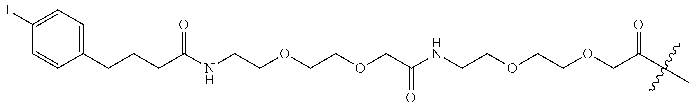 [SMILES: *C(=O)COCCOCCNC(=O)COCCOCCNC(=O)CCCc1ccc(I)cc1] |
| MeCO | 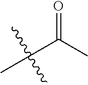 [SMILES: *C(C)=O] |
| mPEG3CO | —C(O)CH₂O(CH₂CH₂O)₂CH₃ |
| mPEG6CO | —C(O)(CH₂CH₂O)₆CH₃ |

### Methods of Making the Cyclic Peptides of the Present Invention

The cyclic peptides of the present invention can be made by Solid-Phase Peptide Synthesis (SPPS) using Fmoc/t-Bu chemistry and purified according to methods known in the art, e.g. according to the methods in E. Atherton and R.C. Sheppard, Solid Phase Peptide Synthesis: a Practical Approach, IRL Press (Oxford, England (1989)); or Amblard et al., Methods and Protocols of Modern Solid Phase Peptide Synthesis, Molecular Biotechnology, 2006, Volume 33, 239-254. Amino acid protecting groups that can be used in the synthesis of the BMPR2 cyclic peptides are summarized in Table A; or Isidro-Llobet et al., Amino Acid-Protecting Groups, Chem. Rev. 2009, 109, 2455-2504, which also describes examples of orthogonal protection schemes that can be used to conjugate, e.g. a natural polymer or unnatural polymer or combination thereof, to a specific site.

In general, the natural polymer or unnatural polymer or combination thereof (e.g, a hydrophilic or water-soluble polymer) can be conjugated to the first or second BMPR2 cyclic peptides by means of N-hydroxy succinimide (NHS)—, click-, or maleimide-based chemistry or other chemistry, as is known in the art. Canalle et al., Polypeptide-polymer bioconjugates, Chem. Soc. Rev., 2010, 39, 329-353; and Gauthier et al., Peptide/protein-polymer conjugates: synthetic strategies and design concepts, Chem. Commun., 2008, 2591-2611, provide overviews of some approaches to constructing peptide-polymer bioconjugates.

Further details on how the BMPR2 cyclic peptide conjugates can be made are provided in the following examples section below.

### General Peptide Synthetic Procedure 1

TL-23R inhibitor compounds described herein were synthesized from amino acids monomers using Merrifield solid phase synthesis techniques on Protein Technology's Symphony multiple channel synthesizer. The peptides were assembled using HBTU (0-Benzotriazole-N,N,N′,N′-tetramethyl-uronium-hexafluoro-phosphate), Diisopropylethylamine(DIEA) coupling conditions. For some amino acid couplings PyAOP(7-Azabenzotriazol-1-yloxy)tripyrrolidinophosponium hexafluorophosphate) and DIEA conditions were used. Rink Amide MBHA resin (100-200 mesh, 0.57 mTrmol/g) was used for peptide with C-terminal amides and pre-loaded Wang Resin with N-cl-Fmoc protected amino acid was used for peptide with C-terminal acids. The coupling reagents (HBTU and DIEA premixed) were prepared at 100 mmol concentration. Similarly, amino acids solutions were prepared at 100 mmol concentration. Peptide inhibitors of the present invention were identified based on medical chemistry optimization and/or phage display and screened to identify those having superior binding and/or inhibitory properties.

### Preparation of Certain Modified Amino Acids

### Synthesis of Protected 7-(3-Nacetyl-phenyl)-tryptoPhan (7(3NAcPh)W) (S)-2-((((9H-fluoren-9-yl)methoxy)carbonyl)amino)-3-(7-(3-acetamidophenyl)-1H-indol-3-yl)propanoic acid

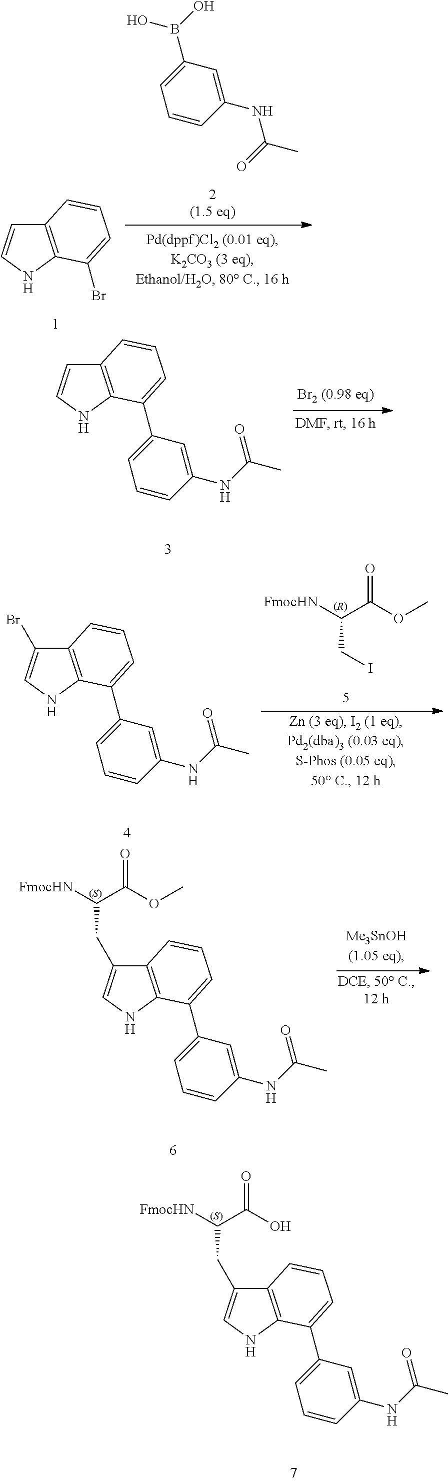

To a solution of 1 (30.0 g, 153 mmol), compound 2 (41.1 g, 230 mmol) and K3PO4 (97.4 g, 459 mmol) in H₂O/ethanol (500 mL) and, Pd(dppf)C12 (1.12 g, 1.53 mmol) was added under an N₂ atmosphere. The mixture was stirred at 80° C. for 16 h. The mixture was filtered. The mixture was concentrated, then extracted with ethyl acetate (500 mL×2), dried with anhydrous Na₂SO₄. The organic layer was concentrated and purified by FCC (eluent: petroleum ether/ethyl acetate=1:0 to 55:45) to give 3 (25.0 g, yield: 62.5%) as yellow oil MS (ESI): mass calculated for C₁₆H₁₄N₂O, 250.295, m/z found 251.0 [M+].

To a 1 L round-bottomed flask containing a solution of 3 (12.0 g, 47.9 mmol) in DMF (300 mL) bromine (Br₂, 2.422 mL, 47.0 mmol) was slowly added. The mixture was stirred at 25° C. for 16 hours. The solution was added to aqueous sodium sulfite (500 mL), the mixture was stirred at 25° C. for 2 hours. The mixture was filtered, the filter cake was mixed with H₂O (400 mL) and stirred at 25° C. for 1 h. The mixture was filtered, the solid was collected to give 4 as a crude product, which was purified by preparative high-performance liquid chromatography (Column: Phenomenex C18 250×50 mm×10 um, Condition: water (FA)-CAN (20%- 60%)). The mixture was concentrated, extracted with CH₂Cl₂ (1 L×2), washed with brine, dried with anhydrous Na₂SO₄. The organic layers was filtered and concentrated to give 4 (9.70 g, yield: 60.8%) as a pale white. MS (ESI): mass calculated for C₁₆H₁₃BrN₂O, 329.191, m/z found 328.8 [M].

A 250 mL three neck round-bottomed flask was charged with activated Zn powder (5.84 g, 89.3 mmol), DMF (120 mL) and I₂ (382 mg, 1.50 mmol) was added under an N₂ atmosphere at room temperature. After stirring for 20 min, a solution of 5 (13.6 g, 30.1 mmol) in DMF (30 mL) was added to the mixture. The reaction mixture was stirred for 30 min at room temperature, after which 4 (9.70 g, 29.5 mmol), tris(dibenzylideneacetone)-palladium (826 mg, 0.902 mmol), 2-dicyclohexylphosphino-2′,6′-dimethoxybiphenyl (617 mg, 1.50 mmol) were added under an N₂ atmosphere. The reaction mixture was stirred at 50° C. for 12 hours, after which solvent was removed under reduced pressure to give crude product 6. The crude product was extracted with ethyl acetate (1500 mL). The extract was washed with H₂O (500 mL×2), followed by brine (500 mL), after which it was dried over anhydrous Na₂SO₄, filtered, and concentrated to dryness in vacuo to give crude intermediate 6, which was purified by silica gel chromatography (0-100% ethyl acetate/petroleum ether (EtOAc/PE)) to afford 6 (11.0 g, yield: 63.8%) as a brown-yellow oil. MS (ESI): mass calculated for C₃₅H₃₁N₃O₅, 573.638, m/z found 574.1 [M+1]

Intermediate 6 (11.0 g, 19.2 mmol), a stir bar, Me₃SnOH (3.64 g, 20.1 mmol) and DCE (150 mL) were added to a 250 mL round-bottomed flask and stirred at 50° C. for 12 hours. To the reaction mixture 2 N HCl was added to adjust the to pH to 6 A second reaction starting from intermediate 6 was conducted and the products were combined for further workup. The combined reaction mixture was concentrated under reduced pressure to give the crude, which was purified by preparative HPLC using a Xtimate C18 150×40 mm×Sum (eluent: 38% to 68% (v/v) CH₃CN and H₂O with 0.05% HCl) to afford product 7. The product was suspended in water (100 mL), the mixture frozen using dry ice/ethanol, and then lyophilized to dryness to afford 7 (7(3NAcPh)W, 11.8 g, yield: 66.8%) as a white solid. MS (ESI): mass calculated For C₃₄H₂₉N₃O₅, 559.611, m/z found 560.0 [M+1]. 1H NMR DMSO-d₆ (400-MHz) δ 10.73 (s, 1H)—10.10 (s, 1H), 7.52-−8.02 (m, 7H), 6.96-7.52 (m, 9H), 4.03-4.4- (m, 3H), 3.25 (d, J=13.2 Hz, 2H), 3.01-3.15 (m, 1H), 2.08 (s, 3H).

### Synthesis of Protected 5-methyl-pyridyl-alanine (5MePyridinAla)

### (S)-2-((((9H-fluoren-9-yl)methoxy)carbonyl)amino)-3-(5-methylpyridin-3-yl)propanoic acid

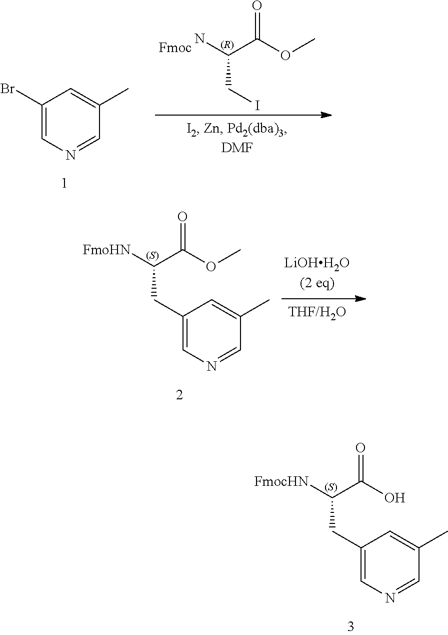

Activated Zn powder (8.18 g, 125 mmol), DMF (150 mL) and I₂ (0.534 g, 2.11 mmol) were stirred under an N₂ atmosphere at room temperature for 20 min., after which (R)-methyl 2-((((9H-fluoren-9-yl)methoxy)carbonyl)amino)-3-iodopropanoate (19.0 g, 42.1 mmol) in DMF (25 mL) was added. The reaction mixture was stirred for 30 min at room temperature, after which a mixture of 1 (7.97 g, 46.3 mmol), tris(dibenzylideneacetone)-palladium (1.16 g, 1.26 mmol) and 2-dicyclohexylphosphino-2′,6′-dimethoxybiphenyl (0.864 g, 2.11 mmol) in DMF (25 mL) was added under an N₂ atmosphere. The resulting reaction mixture was stirred at 50° C. for 12 h. The solvent was removed under reduced pressure to give the crude, which was purified by FCC (eluent: petroleum ether: ethyl acetate=1: 0 to 0: 1 and ethyl acetate: methanol=1: 0 to 2: 1) to afford the product 2 (10.00 g, 57.0% yield) as a pale yellow liquid. MS (ESI): mass calculated for C₂₅H₂₄N₂O₄, 416.469, m/z found 417.1 [M+H]⁺.

To a mixture of 2 (9.50 g, 22.8 mmol) in THE (100 mL) was added LiOH·H₂O (1.91 g, 45.6 mmol) in H₂O (10 mL). The mixture was stirred for 1 h at 0° C. TLC showed most SM were consumed. To the reaction mixture was added HCl (1 N) dropwise at ice bath to pH=5. The reaction mixture was concentrated under reduced pressure, then poured into water (200 mL) the mixture was extracted with THE (200 mL×3). The organic layers were combined, washed with brine (100 mL), dried over anhydrous Na₂SO₄. After filtering the organic layers were concentrated under reduced pressure to afford crude product 3, which was purified by FCC (eluent: ethyl acetate: methanol=1:0 to 2:1) to obtain 3 (5MePyridinAla, 6.716 g, yield: 72.3%) as a white powder. MS (ESI): mass calculated For C₂₄H₂₂N₂O₄, 402.442, m/z found 403.1 [M+H]⁺. ¹H NMR DMSO-d₆ (Bruker_400 MHz): δ 8.18 (s, 2H), 7.88 (d, J=7.6 Hz, 2H), 7.63 (d, J=7.2 Hz, 2H), 7.45-7.26 (m, 5H), 6.81 (s, 1H), 4.33-4.21 (m, 1H), 4.20-4.09 (m, 2H), 3.95 (s, 1H), 3.06-3.05 (m, 1H), 2.92-2.89 (m, 1H), 2.18 (s, 3H).

### EXAMPLE 6. Synthesis of Protected AEF(G)

### (S)-2-((((9H-fluoren-9-yl)methoxy)carbonyl)amino)-3-(4-(2-(3-((2,2,4,6,7-pentamethyl-2,3-dihydrobenzofuran-5-yl)sulfonyl)guanidino)ethoxy)phenyl)propanoic acid

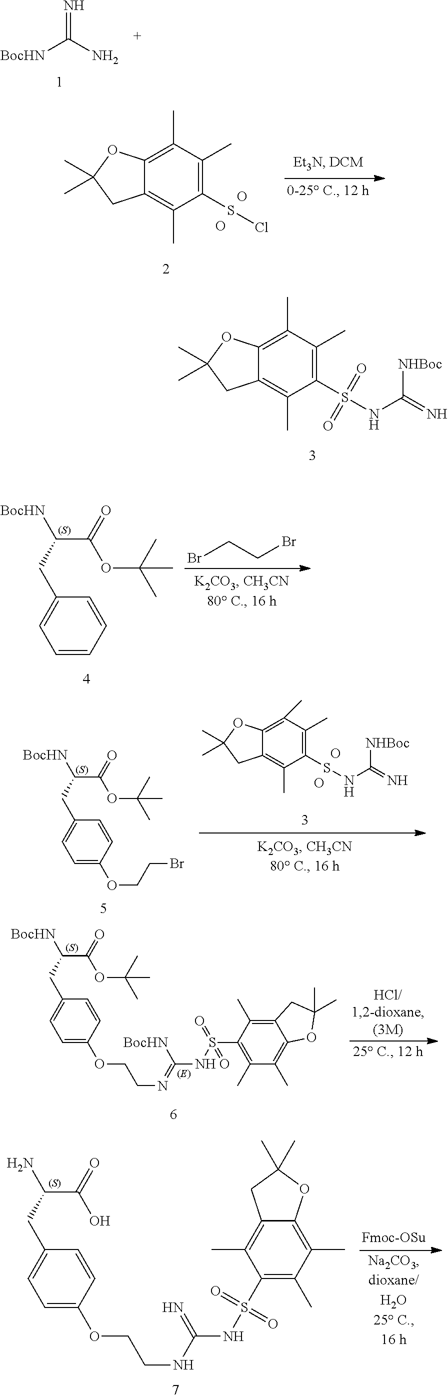

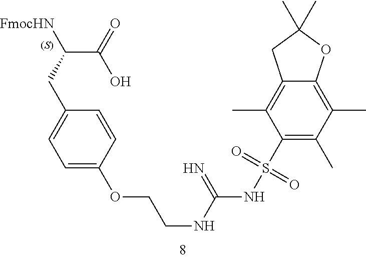

Starting material 1 (9.9 g, 62.2 mmol), a stir bar, Et₃N (14 mL, 101 mmol), and dichloromethane (DCM, 250 mL) were added to a 500 mL round-bottomed flask. The resulting mixture was treated with 2 (10 g, 34.6 mmol) in portions under ice-water bath. Then the reaction mixture was stirred at 25° C. for 12 hours. The reaction mixture was diluted with H₂O (800 mL), extracted with DCM (400 mL×2). The organic phase extracts were combined, washed with brine (800 mL), and concentrated to give the crude intermediate 3 as a yellow solid. The crude intermediate was triturated with ethyl acetate (50 mL) and the suspension isolated via filtration. The filter cake was washed with ethyl acetate (20 mL×3) before drying under reduced pressure to give the 3 (7.12 g, 49%) as a white solid. MS (ESI): mass calculated for C₁₉H₂₉N₃O₅S₆, 411.5, m/z found 412.1 [M+H]⁺.

Starting material 4 (50.0 g, 148 mmol), a stir bar, DMF (300 mL), and K₂CO₃(102 g, 739 mmol) were added to a nitrogen-purged 1000 mL round-bottomed flask. The flask was subsequently evacuated and refilled with nitrogen (×3), after which 1,2-dibromoethane (154 mL, 1.78 mol) was added, and the resulting mixture was stirred at 80° C. for 16 h under a N₂ atmosphere. The reaction mixture was filtered and concentrated to dryness under reduced pressure to give the crude product, which was subjected to silica gel chromatography (eluent: EtOAc: pet ether =0-60%) to give the 5 (64 g, 96%) as a light yellow oil. MS (ESI): mass calculated for C₂₀H₃₀BrNO₅, 444.36, m/z found 466.1 [M+Na]⁺.

Intermediate 5 (6.1 g, 13.7 mmol), 3 (6.2 g, 15.1 mmol), K₂CO₃ (7.6 g, 55.0 mmol), a stir bar, and CH₃CN (100 mL) were charged into a 250 mL round-bottomed flask. The reaction mixture was stirred at 80° C. for 16 h under a N₂ atmosphere. The reaction mixture was cooled to room temperature, diluted with H₂O (200 mL), extracted with ethyl acetate (100 mL×2). The organic phases were combined and washed with brine (300 mL) and concentrated to give the crude intermediate 6. The crude intermediate was purified by flash column chromatography (FCC, eluent: ethyl acetate/petroleum ether=0:1 to 2:1) to give the 6 (6.62 g, 44.2%) as a white solid. MS (ESI): mass calculated for C₃₉H₅₈N₄O₁₀S, 774.9, m/z found 775.5 [M+H]⁺

Intermediate 6 (6.6 g, 8.52 mmol), HCl/1,4-dioxane (90 mL, 4M), a stir bar, and 1,4-dixoane (30 mL) were charged into a 250 mL round bottomed flask. The resulting mixture was stirred at 25° C. for 12 hr. The solvent was removed under reduced pressure to give intermediate 7 (7.8 g, crude product) as a colourless oil, which was directly used to next step. MS (ESI): mass calculated for C₂₅H₃₄N₄O₆S, 518.6, m/z found 519.2 [M+H]⁺

Intermediate 7 (7.80 g, 15.0 mmol), a stir bar, Na₂CO₃ (3.19 g, 30.1 mmol), Fmoc-OSu (5.58 g, 16.5 mmol), 1, 4-dioxane (50 mL), and H₂O (50 mL) were added into a 250 mL round-bottomed flask at 25° C. The reaction mixture was stirred at 25° C. for 16 hours, after which it was adjusted to pH=5-6 with HCl (2M) and the resulting reaction mixture was extracted with EtOAc (150 mL×3). The organic phases from the extraction were combined and washed with brine (200 mL) and concentrated to give the crude intermediate 7. The crude intermediate was purified by preparative HPLC with a Column: Phenomenex C18 150×40 mm×Sum, (eluent: 42% to 72% (v/v) CH₃CN and H₂O with 0.1% HCl) to afford pure product. The product was suspended in water (100 mL), the mixture frozen using dry ice/ethanol, and then lyophilized to dryness to afford desired product 8 AEF(G), 4 g, 36%) as a white solid. MS (ESI): mass calculated for C₄₀H₄₄N₄O₈S, 740.9, m/z found 741.3 [M+H]⁺. ¹H NMR (400 MHz, DMSO-d₆): 7.87 (d, J=7.2 Hz, 2H), 7.71-7.62 (m, 2H), 7.39 (td, J=4.0, 7.2 Hz, 2H), 7.29 (td, J=7.6, 12.0 Hz, 2H), 7.14 (br d, J=8.0 Hz, 2H), 6.99-6.85 (m, 1H), 6.77 (br d, J=8.4 Hz, 2H), 6.59-6.50 (m, 1H), 4.21-4.06 (m, 4H), 3.88 (br s, 2H), 3.42-3.36 (m, 4H), 2.99 (br dd, J=4.4, 14.0 Hz, 1H), 2.92 (s, 2H), 2.78 (br dd, J=10.8, 13.6 Hz, 1H), 2.47 (br s, 3H), 2.41 (s, 3H), 1.97 (s, 3H), 1.38 (s, 6H).

### Assembly

The peptides were assembled using standard Symphony protocols. The peptide sequences were assembled as follows: Resin (250 mg, 0.14 mmol) in each reaction vial was washed twice with 4 ml of DMF followed by treatment with 2.5 ml of 20% 4-methyl piperidine (Fmoc de-protection) for 10 min. The resin was then filtered and washed two times with DMF (4 ml) and re-treated with N-methyl piperidine for additional 30 minute. The resin was again washed three times with DMF (4 ml) followed by addition 2.5 ml of amino acid and 2.5 ml of HBTU-DIEA mixture. After 45 min of frequent agitations, the resin was filtered and washed three timed with DMF (4 ml each). For a typical peptide of the present invention, double couplings were performed. After completing the coupling reaction, the resin was washed three times with DMF (4 ml each) before proceeding to the next amino acid coupling.

Ring Closing Metathesis to form Olefins

An an example of ring closing metathesis a the resin (100 mol) was washed with 2 ml of DCM (3×1 min) and then with 2 ml of DCE (3×1 min) before being treated with a solution of 2 ml of a 6 mM solution of Grubbs' first-generation catalyst in DCE (4.94 mg ml-1; 20 mol % with regard to the resin substitution). The solution was refluxed overnight (12 h) under nitrogen before being drained. The resin was washed three times with DMF (4 ml each); DCM (4 ml) before being dried and cleaved.

### Cleavage

Following completion of the peptide assembly, the peptide was cleaved from the resin by treatment with cleavage reagent, such as reagent K (82.5% trigluoroacetic acid, 5% water, 5% thioanisole, 5% phenol, 2.5% 1,2-ethanedithiol). The cleavage reagent was able to successfully cleave the peptide from the resin, as well as all remaining side chain protecting groups.

The cleaved peptides were precipitated in cold diethyl ether followed by two washings with ethyl ether. The filtrate was poured off and a second aliquot of cold ether was added, and the procedure repeated. The crude peptide was dissolved in a solution of acetonitrile:water (7:3 with 1% TFA) and filtered. The quality of linear peptide was then verified using electrospray ionization mass spectrometry (ESI-MS) (Micromass/Waters ZQ) before being purified.

### Disulfide Bond Formation Via Oxidation

The peptide containing the free thiol (for example diPen) was assembled on a Rink Amide-MBHA resin following general Fmoc-SPPS procedure. The peptide was cleaved from the resin by treatment with cleavage reagent 90% trifluoroacetic acid, 5% water, 2.5% 1,2-ethanedithiol, 2.5% tri-isopropylsilane). The cleaved peptides were precipitated in cold diethyl ether followed by two washings with ethyl ether. The filtrate was poured off and a second aliquot of cold ether was added, and the procedure repeated. The crude peptide was dissolved in a solution of acetonitrile:water (7:3 with 1% TFA) and filtered giving the wanted unoxidized peptide crude peptide.

The crude, cleaved peptide was dissolved in 20 ml of water: acetonitrile. Saturated Iodine in acetic acid was then added drop wise with stirring until yellow color persisted. The solution was stirred for 15 minutes, and the reaction was monitored with analytic HPLC and LCMS. When the reaction was completed, solid ascorbic acid was added until the solution became clear. The solvent mixture was then purified by first being diluted with water and then loaded onto a reverse phase HPLC machine (Luna C18 support, 10u, 100A, Mobile phase A: water containing 0.1% TFA, mobile phase B: Acetonitrile (ACN) containing 0.1% TFA, gradient began with 5% B, and changed to 50% B over 60 minutes at a flow rate of 15 ml/min). Fractions containing pure product were then freeze-dried on a lyophilyzer.

### Thioether Bond Formation

The peptide containing the free thiol was assembled on a Rink Amide-MBHA resin following general Fmoc-SPPS procedure. Chlorination was carried out by treating the resin with PPh₃ (10 equiv.) and C₁₃CCN (10 equiv.) in DCM for 2 h. The peptide was cleaved from the resin by treatment with cleavage reagent 90% trifluoroacetic acid, 5% water, 2.5% 1,2-ethanedithiol, 2.5% tri-isopropylsilane). The cleaved peptides were precipitated in cold diethyl ether followed by two washings with ethyl ether. The filtrate was poured off and a second aliquot of cold ether was added, and the procedure repeated. The crude peptide was dissolved in a solution of acetonitrile:water (7:3 with 1% TFA) and filtered giving the wanted uncyclized crude peptide

### Purification

Analytical reverse-phase, high performance liquid chromatography (HPLC) was performed on a Gemini C18 column (4.6 mm×250 mm) (Phenomenex). Semi-Preparative reverse phase HPLC was performed on a Gemini 10 m C18 column (22 mm×250 mm) (Phenomenex) or Jupiter 10 m, 300 angstrom (A) C18 column (21.2 mm×250 mm) (Phenomenex). Separations were achieved using linear gradients of buffer B in A (Mobile phase A: water containing 0.15% TFA, mobile phase B: Acetonitrile (ACN) containing 0.1% TFA), at a flow rate of 1 mL/min (analytical) and 15 mL/min (preparative). Separations were achieved using linear gradients of buffer B in A (Mobile phase A: water containing 0.15% TFA, mobile phase B: Acetonitrile (ACN) containing 0.1% TFA), at a flow rate of 1 mL/min (analytical) and 15 mL/min (preparative).

### General Procedure 1A:

TL-23R inhibitor compounds described herein were synthesized from amino acids monomers using standard Fmoc solid phase synthesis techniques on a CEM Liberty Blu™ microwave peptide synthesizer. The peptides were assembled using Oxyma/DIC (ethyl cyanohydroxyiminoacetate/diisopropyl-carbodiimide) with microwave heating. Rink Amide-MB- A resin (100-200 mesh, 0.66 mmol,/g) was used for peptides with C-terminal amides and pre-loaded Wang Resin with N-α-Fmoc protected amino acid was used for peptide with C-terminal acids. Oxyma was prepared as a 1M solution in DMF with 0.1 M DIEA. DIC was prepared as 0.5M solution in DMF. The Amino acids were prepared at 200 mM. Peptide inhibitors of the present invention were identified based on medicinal chemistry optimization and/or phage display and screened to identify those having superior binding and/or inhibitory properties.

### Assembly

The peptides were made using standard CEM Liberty Blue™ protocols. The peptide sequences were assembled as follows: Resin (400 mg, 0.25 mmol) was suspended in 10 ml of 50/50 DMF/DCM. The resin was then transferred to the reaction vessel in the microwave cavity. The peptide was assembled using repeated Fmoc deprotection and Oxyma/DIC coupling cycles. For deprotection, 20% 4-methylpiperidine in DMF was added to the reaction vessel and heated to 90° C. for 65 seconds. The deprotection solution was drained and the resin washed three times with DMF. For most amino acids, 5 equivalents of amino acid, Oxyma and DIC were then added to the reaction vessel and microwave irradiation rapidly heated the mixing reaction to 90° C. for 4 min. For Arginine and Histidine residues, milder conditions using respective temperatures of 75 and 50° C. for 10 min were used to prevent racemization. Rare and expensive amino acids were often coupled manually overnight at room temperature using only 1.5-2 eq of reagents. Difficult couplings were often double coupled 2×4 min at 90° C. After coupling the resin was washed with DMF and the whole cycle was repeated until the desired peptide assembly was completed.

### Cleavage

Following completion of the peptide assembly, the peptide was then cleaved from the resin by treatment with a standard cleavage cocktail of 91:5:2:2 TFA/H₂O/TIPS/DODT for 2 hrs. If more than one Arg(pbf) residue was present the cleavage was allowed to go for an additional hour.

The cleaved peptides were precipitated in cold diethyl ether. The filtrate was decanted off and a second aliquot of cold ether was added, and the procedure was repeated. The quality of linear peptide was then verified using electrospray ionization mass spectrometry (ESI-MS) (Waters® Micromass® ZQ™) before being purified.

Disulfide Bond Formation via Oxidation

The peptide containing the free thiol (for example diPen) was assembled on a Rink Amide-MBHA resin following general Fmoc solid phase synthesis, cleavage and isolation as described above.

The crude cleaved peptide comprising two thiol containing amino acids was dissolved −2 mg/ml in 50/50 acetonitrile/water. Saturated iodine in acetic acid was then added dropwise with stirring until yellow color persisted. The solution was stirred for a few minutes, and the reaction was monitored with analytic HPLC and LCMS. When the reaction was completed, solid ascorbic acid was added until the solution became clear. The solvent mixture was then purified by first being diluted with water and then loaded onto a reverse phase HPLC Column (Luna® C18 support, 10u, 100A, Mobile phase A: water containing 0.1% TFA, mobile phase B: acetonitrile (ACN) containing 0.1% TFA, gradient began with 15% B, and changed to 50% B over 60 minutes at a flow rate of 15 ml/min). Fractions containing pure product were then freeze-dried on a lyophilizer.

### Purification

Analytical reverse-phase, high performance liquid chromatography (HPLC) was performed on a Gemini® C18 column (4.6 mm×250 mm) (Phenomenex). Semi-Preparative reverse phase HPLC was performed on a Gemini® 10 m C18 column (22 mm×250 mm) (Phenomenex) or Jupiter® 10 m, 300 angstrom (A) C18 column (21.2 mm×250 mm) (Phenomenex). Separations were achieved using linear gradients of buffer B in A (Mobile phase A: water containing 0.15% TFA, mobile phase B: Acetonitrile (ACN) containing 0.1% TFA), at a flow rate of 1 mL/min (analytical) and 20 mL/min (preparative).

### Alternate Procedure for Solid-Phase Synthesis of Peptides

Peptide were chemically synthesized using optimized 9-fluorenylmethoxy carbonyl (Fmoc) solid phase peptide synthesis protocols. For C-terminal amides, Rink-amide MBHA resin, Methyl Indole AM resin or CTC resin then couple amine after cleavage was used. The side chain protecting groups were as follows: Thr, Glu: 0-tButyl; Asn, Pen: Trityl; AEF, AEF(NMe): Boc; D-Arg: Pbf For coupling, a two to five-fold excess of a solution containing Fmoc amino acid, HATU and DIEA (1:0.95:2) in DMF was added to swelled resin for 1 to 5 hours. Double coupling is employed when coupling 2Nal or other sterically hindered amino acids. Fmoc protecting group removal was achieved by treatment with a DMF, piperidine (4:1) solution for 30 min. The cycles are repeated until the full-length peptide is obtained. The quaternary ammonium amino acids (eg: APEG3F) should be transferred to HCl salt if the salt form was formic acid.

### Procedure for Cleavage of Peptides Off Resin

Side chain deprotection and cleavage of the peptide analogues of the invention was achieved by stirring dry resin in a solution containing trifluoroacetic acid, water, DTT and tri-isopropylsilane (90:2.5:5:2.5) for 3 hours. Filtered and the combined filtrate was precipitated with cold methyl tertbutyl ether (MTBE). The mixture was centrifuged (3000 rpm, 3 min) and decanted. The pellet was washed with MTBE and centrifuged. Lyophilized the residue to give the linear peptide.

### Procedure for Cyclization

For cyclization, the iodine solution in MeOH (0.1M) was added to peptide solution (20% MeCN/H2O (1mmol/L)) drop-wise until solution remains yellow. After ˜2h LCMS showed the reaction was complete. Excess iodine was quenched by the addition of 1M Na2S2O3 in water (turned colorless instantly).

### Procedure for Purification of Peptides

Purification of the peptides was achieved using reverse-phase high performance liquid chromatography (RP-HPLC). Purification of the cyclized peptides was achieved using preparative RP-HPLC with a C18 column with a flow rate of 20 mL/min. Separation was achieved using gradients of buffer B in A (Buffer A: 0.075% TFA in water; Buffer B: ACN). (Note 1). Analysis was performed using a C18 column with a flow rate of 1 mL/min (Note 2).

One of skill in the art will appreciate that standard methods of peptide synthesis may be used to generate the compounds of the invention.

Prep. HPLC Method A: Description: Mobile Phase: 0.075% TFA in water (solvent A) and acetonitrile (solvent B) Column: Luna 100*25 mm, C18, 10 um, 100 Å+Gemini® 150*30 mm, C18, 5 um, 110A column; Flow Rate: 20 mL/min; Wavelength: UV 220 nm&254 nm; Oven Tem. Room temperature

Prep. HPLC Method B: Description: Mobile Phase: 0.075% TFA in water (solvent A) and acetonitrile (solvent B) Column: Welch Ultimate XB—C18, 250*50 mm, 7 um, 120 Å+Welch Xtimate C18, 250*50 mm, 10 um, 120A column; Flow Rate: 80 mL/min; Wavelength: UV 220 nm&254 nm; Oven Tem. Room temperature

Prep. HPLC Method C: Description: Mobile Phase: 0.075% TFA in water (solvent A) and acetonitrile (solvent B) Column: Luna C18, 250*100 mm, 10 um, 100A column; Flow Rate: 120 mL/min; Wavelength: UV 220 nm&254 nm; Oven Tem. Room temperature

Prep. HPLC Method D: Description: Mobile Phase: 0.5% AcOH in water (solvent A) and acetonitrile (solvent B) Column: Luna C18, 250*100 mm, 10 um, 100A column; Flow Rate: 120 mL/min; Wavelength: UV 220 nm&254 nm; Oven Tem. Room temperature

Prep. HPLC Method E: Description: Mobile Phase: 0.075% TFA in water (solvent A) and acetonitrile (solvent B) Column: Welch Ultimate® XB—C18, 250*100 mm, 10 um, 120A column; Flow Rate: 250 mL/min; Wavelength: UV 220 nm&254 nm; Oven Tem.

### Room Temperature

Prep. HPLC Method F: Description: Mobile Phase: 0.5% AcOH in water (solvent A) and acetonitrile (solvent B) Column: Welch Ultimate® XB—C18, 250*50 mm, 10 um, 120 Å+Welch Xtimate®C18, 250*50 mm, 10 um, 120A column; Flow Rate: 80 mL/min; Wavelength: UV 220 nm&254 nm; Oven Tem. Room temperature

Analysis LCMS Method: Mobile Phase: 0.1% TFA in water (solvent A) and 0.1% TFA in acetonitrile (solvent B), using the elution gradient 10%-80% (solvent B) over 0.9 minutes and using the elution gradient 80%-90% for 0.6 minutes at a flow rate of 1.0 ml/min; Column: Xbrige C18,3.5 um, 2.1*30 mm; Wavelength: UV 220 nm&254 nm; Column temperature: 30° C.; MS ionization: ESI

### Examples

### EXAMPLE 1: SYNTHESIS OF SEQ ID 57

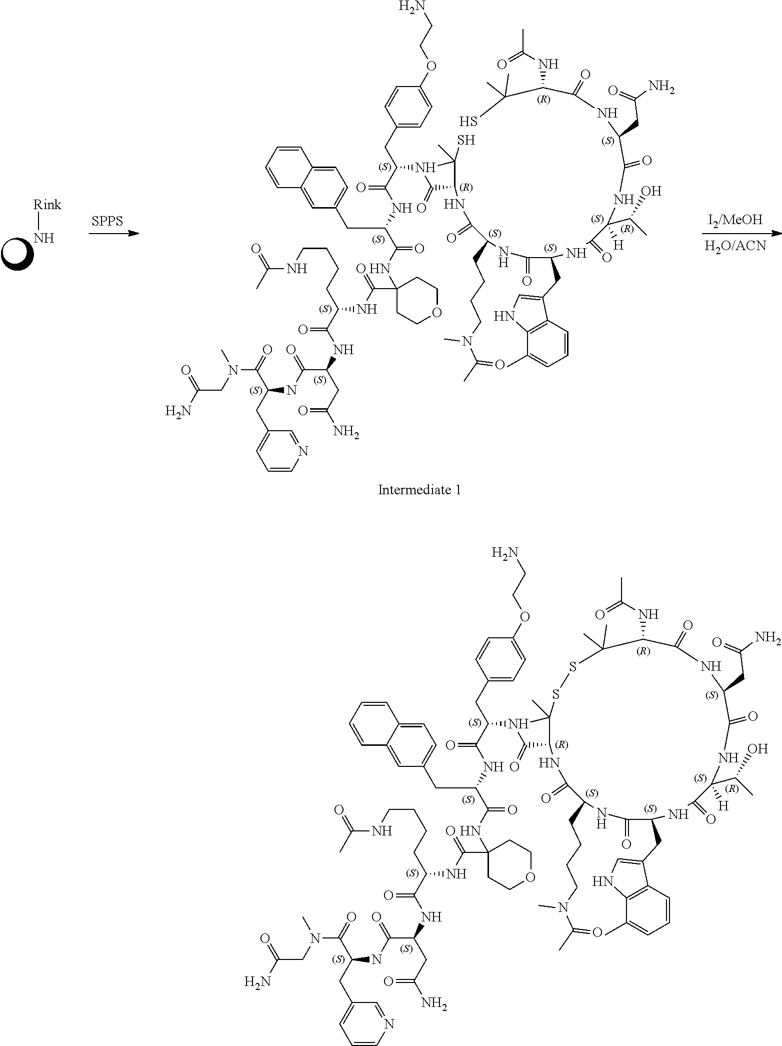

The peptide was synthesized using standard Fmoc chemistry.
1) Add DMF to the vessel containing MBHA Resin (0.20 mmol, 0.64 g, sub: 0.31 mmol/g) and swell for 1-4 hours.
2) Add 20% piperidine/DMF and mix for 30 min.
3) Drain and then DMF wash 30 sec with 5 times.
4) Add Fmoc-amino acid solution and mix 30 seconds, then add activation buffer, N₂ bubbling for about 2 hour
5) Add 20% piperidine/DMF and mix for 30 min
6) Repeat step 2 to 5 for next amino acid coupling. The coupling reaction was monitored by ninhydrin or tetrachlor color test, and the resin was washed with DMF for 5 times. The resin was washed with MeOH for 3 times and dried by vacuum

Monitored method:
1. Ninhydrin test: A: 5% ninhydrin/EtOH; B: 80% phenol/EtOH; C: pyridine
2. Tetrachlor color test: A: 2% tetrachlor/DMF; B: 2% aldehyde/DMF 110° C. for 3 min

### Peptide Cleavage:

1) Add cleavage buffer (5.0% DTT/2.5% H₂O/2.5% TIS/90% TFA) 12 mL to the flask containing the side chain protected peptide at room temperature and stir for 3 hrs.
2) Filtered washed with 5 mL TFA. The combined filtrate was precipitated with cold methyl tertbutyl ether (MTBE). The mixture was centrifuged (3000 rpm, 3 min) and decanted. The pellet was washed with MTBE and centrifuged.
3) Lyophilized the residue to give the intermediate 1 (350 mg, crude) Peptide Cyclization and Purification:

Crude peptide intermediate 1 (350 mg, 0.179 mmol) was dissolved in 20% MeCN/H2O (200 mL). To a stirred solution of the peptide was added the iodine solution in MeOH (0.1M, 0.8 mL) drop-wise until solution remains yellow. After ˜2h LCMS showed the reaction was complete. Excess iodine was quenched by the addition of 1M Na₂S₂O₃ in water (15 uL) (turned colorless instantly). Added 10-20 mL of MeCN to decrease turbidity.

Purified the solution by Prep-HPLC (A: 0.075% TFA in H₂O, B: ACN) (Note 1: Method A) to give SEQ ID 57 (122.7 mg, 97.3% purity, 30.6% yield for this step; over all yield: 27.2%) obtained as white solid. Analysis was performed using a C18 column with a flow rate of 1 mL/min (Note 2). LCMS Summary: calculated MW: 1953.29, observed MW: 977.1 [(M+2H)/2]

### Example 2: Synthesis of Seq Id 67

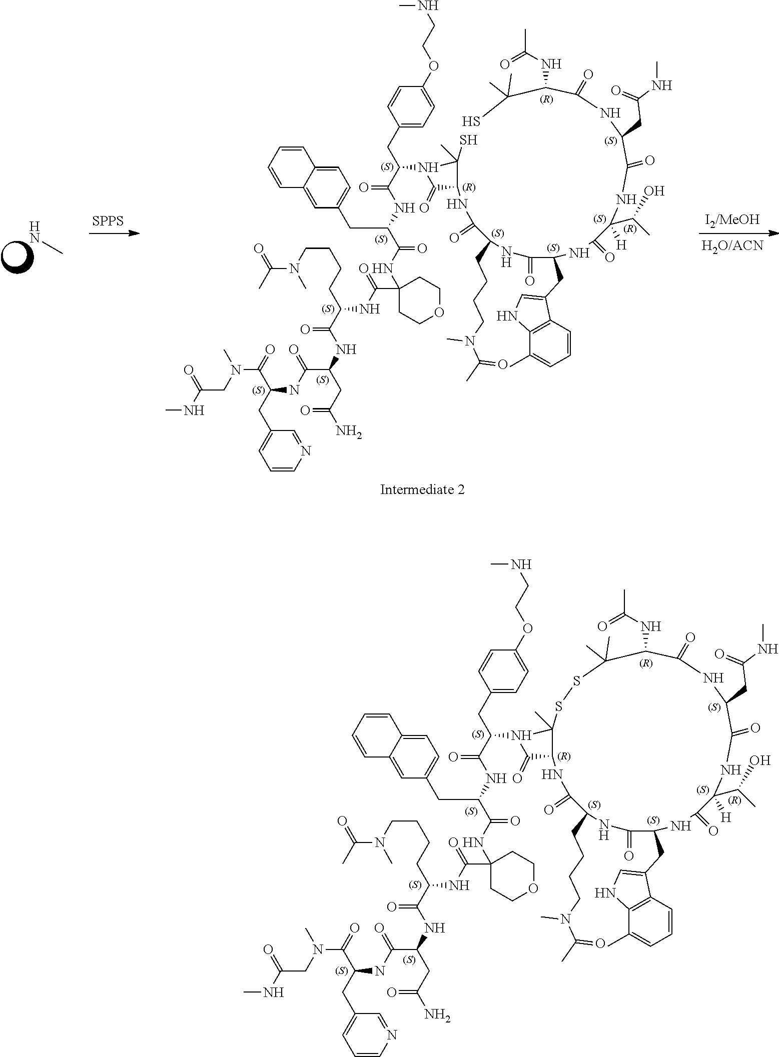

The peptide was synthesized using standard Fmoc chemistry.
1) Add DMF to the vessel containing Methyl Indole AM resin (0.20 mmol, 0.29 g, sub: 0.69 mmol/g) and swell for 2 hours.
2) Add 20% piperidine/DMF and mix for 30 min.
3) Drain and then DMF wash 30 sec with 5 times.
4) Add Fmoc-amino acid solution and mix 30 seconds, then add activation buffer, N₂ bubbling for about 1-4 hours
5) Add 20% piperidine/DMF and mix for 30 min
6) Repeat step 2 to 5 for next amino acid coupling. The coupling reaction was monitored by ninhydrin or tetrachlor color test, and the resin was washed with DMF for 5 times. The resin was washed with MeOH for 3 times and dried by vacuum

Monitored method:
1. Ninhydrin test: A: 5% ninhydrin/EtOH; B: 80% phenol/EtOH; C: pyridine
2. Tetrachlor color test: A: 2% tetrachlor/DMF; B: 2% aldehyde/DMF 110° C. for 3 min

### Peptide Cleavage:

1) Add cleavage buffer (5.0% DTT/2.5% H₂O/2.5% TIS/90% TFA) 12 mL to the flask containing the side chain protected peptide at room temperature and stir for 3 hrs.
2) Filtered washed with 5 mL TFA. The combined filtrate was precipitated with cold methyl tertbutyl ether (MTBE). The mixture was centrifuged (3000 rpm, 3 min) and decanted. The pellet was washed with MTBE and centrifuged.
3) Lyophilized the residue to give the intermediate 2 (350 mg, crude)

### Peptide Cyclization and Purification:

Crude peptide intermediate 2 (350 mg, 0.174 mmol) was dissolved in 20% MeCN/H2O (200 mL). To a stirred solution of the peptide was added the iodine solution in MeOH (0.1M, 1.5 mL) drop-wise until solution remains yellow. After ˜2h LCMS showed the reaction was complete. Excess iodine was quenched by the addition of 1M Na₂S₂O₃ in water (15 uL) (turned colorless instantly). Added 10-20 mL of MeCN to decrease turbidity.

Purified the solution by Prep-HPLC (A: 0.075% TFA in H₂O, B: ACN) (Note 1: Method A) to give SEQ ID 67 (85.9 mg, 97.7% purity, 21.5% yield for this step; over all yield: 18.7%) obtained as white solid. Analysis was performed using a C18 column with a flow rate of 1 mL/min (Note 2). LCMS Summary: calculated MW: 2009.39, observed MW: 1005.0 [(M+2H)/2], 670.3 [(M+3H)/3].

### EXAMPLE 3: SYNTHESIS OF SEQ ID 68

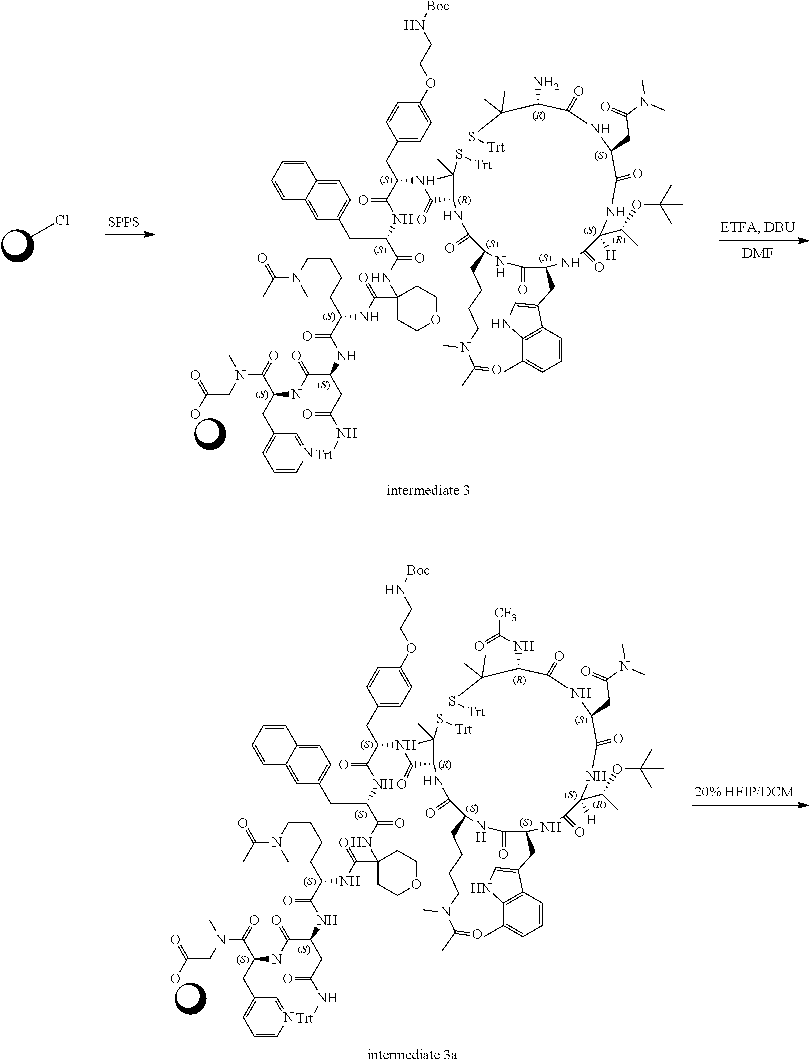

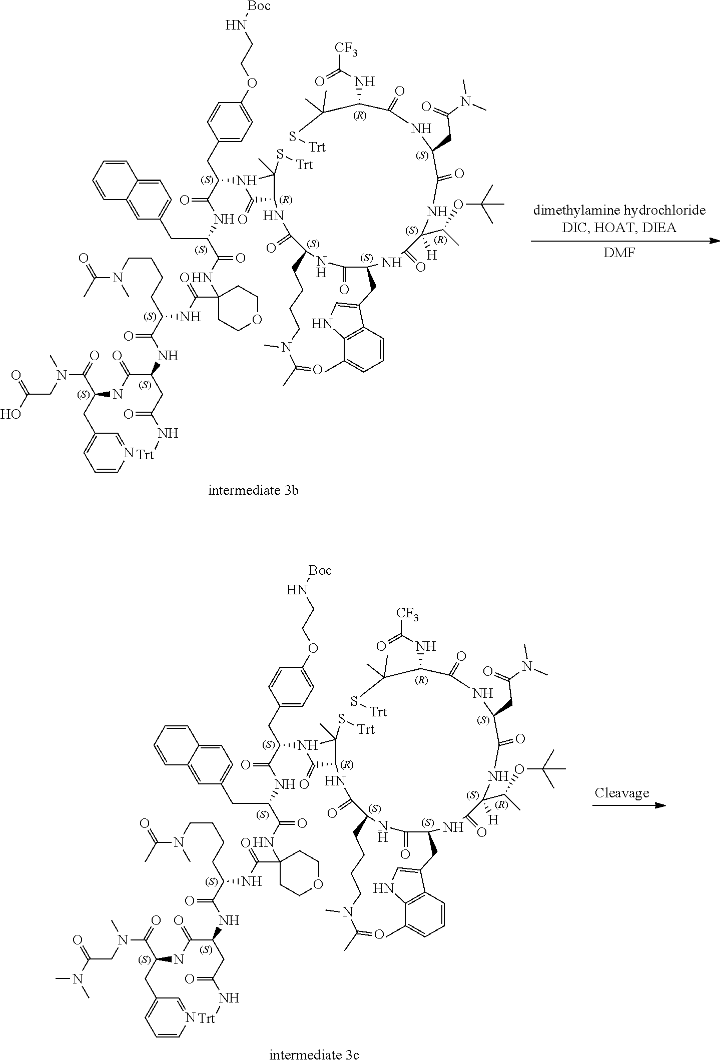

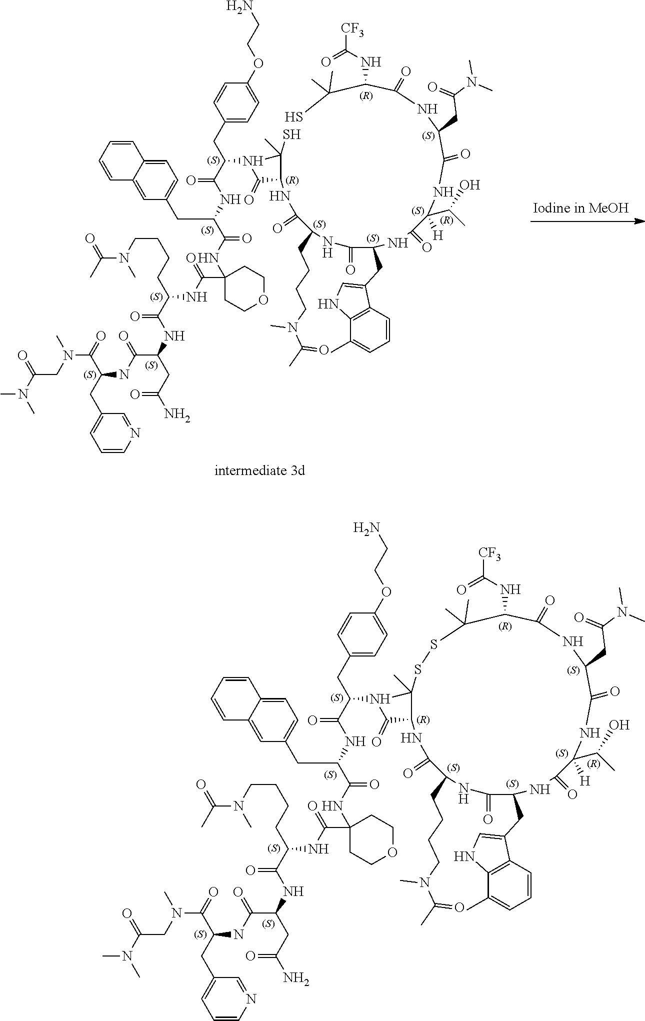

The peptide was synthesized using standard Fmoc chemistry.
1) Add DMF to the vessel containing Sar-CTC Resin (0.30 mmol, 0.96 g, sub: 0.31 mmol/g) and swell for 2 hours.
2) Add 20% piperidine/DMF and mix for 30 min.
3) Drain and then DMF wash 30 sec with 5 times.
4) Add Fmoc-amino acid solution and mix 30 seconds, then add activation buffer, N₂ bubbling for about 1-4 hours
5) Add 20% piperidine/DMF and mix for 30 min
6) Repeat step 2 to 5 for next amino acid coupling. The coupling reaction was monitored by ninhydrin or tetrachlor color test, and the resin was washed with DMF for 5 times. The resin was washed with MeOH for 3 times and dried by vacuum

Monitored method:
1. Ninhydrin test: A: 5% ninhydrin/EtOH; B: 80% phenol/EtOH; C: pyridine 2. Tetrachlor color test: A: 2% tetrachlor/DMF; B: 2% aldehyde/DMF 110° C. for 3 min Peptide Cleavage (General procedure for preparation of intermediate 3b):
1) Add cleavage buffer (20% HFIP/DCM) 50 mL to the peptide Resin stirring for 30 min for 3 times.
2) The DCM was concentrated under reduced pressure.
3) Dissolved the crude peptide in ACN/water and lyophilized overnight. to give the intermediate 3b (300 mg, crude)

### General Procedure for Preparation of Intermediate 3c

To a solution of compound 3b (300 mg, 0.10 mmol) in DMF (5.00 mL) was added dimethylamine hydrochloride (16.3 mg, 2.00 eq), DIC (30.9 uL, 2.00 eq), HOAT (27 mg, 2.00 eq) and DIEA (34 uL, 2.00 eq). The mixture was stirred at 25° C. for 1 h. The reaction was monitored by LCMS, LCMS showed the starting material didn't react completely, then added one equivalent of dimethylamine hydrochloride, DIC, HOAT and DIEA for 16 h, LCMS showed starting material was consumed received desired mass. The filtrate was precipitated with cold methyl tertbutyl ether (MTBE) (50 mL) and centrifuged (3000 rpm, 3 min) to get an oily liquid, the isopropyl ether was dried with nitrogen to get intermediate 3c.

### General Procedure for Preparation of Intermediate 3d

1) Add cleavage buffer (5.0% DTT/2.5% H₂O/2.5% TIS/90% TFA) 30 mL to the flask containing the side chain protected peptide at room temperature and stir for 3 hrs.
2) Filtered washed with 5 mL TFA. The combined filtrate was precipitated with cold methyl tertbutyl ether (MTBE). The mixture was centrifuged (3000 rpm, 3 min) and decanted. The pellet was washed with MTBE and centrifuged.
3) Lyophilized the residue to give the intermediate 3d (350 mg, crude)

### General Procedure for Preparation of SEQ ID 68

Crude peptide intermediate 3d (350 mg, 0.168 mmol) was dissolved in 20% MeCN/H2O (300 mL). To a stirred solution of the peptide was added the iodine solution in MeOH (0.1M, 1.5 mL) drop-wise until solution remains yellow. After ˜2h LCMS showed the reaction was complete. Excess iodine was quenched by the addition of 1M Na₂S₂O₃ in water (15 uL) (turned colorless instantly). Added 10-20 mL of MeCN to decrease turbidity. Purified the solution by Prep-HPLC (A: 0.075% TFA in H₂O, B: ACN) (Note 1: Method A) to give SEQ ID 68 (49.7 mg, 95.0% purity, 12.1% yield for this step; over all yield: 6.83%) obtained as white solid. Analysis was performed using a C18 column with a flow rate of 1 mL/min (Note 2). LCMS Summary: calculated MW: 2077.39, observed MW: 1039.6 [(M+2H)/2], 693.5 [(M+3H)/3].

### Example 4: Synthesis of Seq Id 82

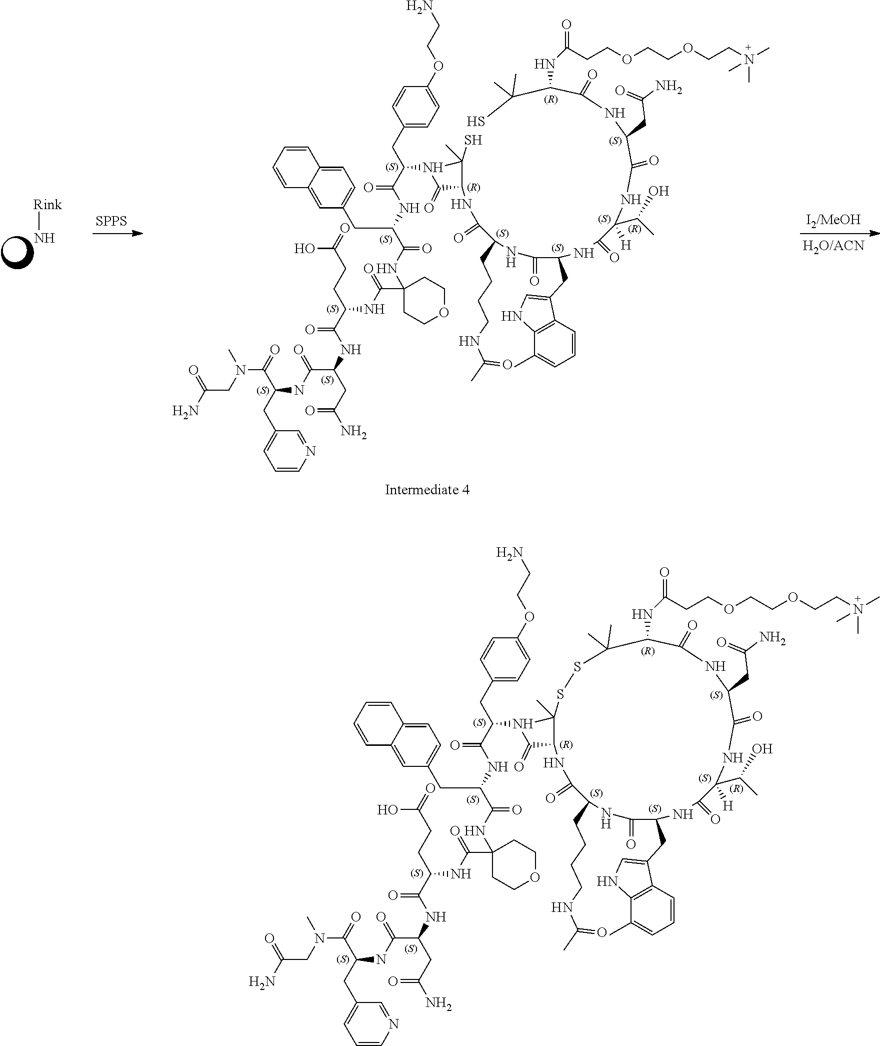

The peptide was synthesized using standard Fmoc chemistry.
7) Add DMF to the vessel containing MBHA Resin (0.20 mmol, 0.64 g, sub: 0.31 mmol/g) and swell for 2 hours.
8) Add 20% piperidine/DMF and mix for 30 min.
9) Drain and then DMF wash 30 sec with 5 times.
10) Add Fmoc-amino acid solution and mix 30 seconds, then add activation buffer, N₂ bubbling for about 1-4 hours
11) Add 20% piperidine/DMF and mix for 30 min
12) Repeat step 2 to 5 for next amino acid coupling. The coupling reaction was monitored by ninhydrin or tetrachlor color test, and the resin was washed with DMF for 5 times. The resin was washed with MeOH for 3 times and dried by vacuum

Monitored method:
1. Ninhydrin test: A: 5% ninhydrin/EtOH; B: 80% phenol/EtOH; C: pyridine
2. Tetrachlor color test: A: 2% tetrachlor/DMF; B: 2% aldehyde/DMF 110° C. for 3 min

### Peptide Cleavage:

4) Add cleavage buffer (5.0% DTT/2.5% H₂O/2.5% TIS/90% TFA) 12 mL to the flask containing the side chain protected peptide at room temperature and stir for 3 hrs.
5) Filtered washed with 5 mL TFA. The combined filtrate was precipitated with cold methyl tertbutyl ether (MTBE). The mixture was centrifuged (3000 rpm, 3 min) and decanted. The pellet was washed with MTBE and centrifuged.
6) Lyophilized the residue to give the intermediate 4 (350 mg, crude)

### Peptide Cyclization and Purification:

Crude peptide intermediate 4 (350 mg, 0.17 mmol) was dissolved in 20% MeCN/H2O (200 mL). To a stirred solution of the peptide was added the iodine solution in MeOH (0.1M, 0.8 mL) drop-wise until solution remains yellow. After ˜2h LCMS showed the reaction was complete. Excess iodine was quenched by the addition of 1M Na₂S₂O₃ in water (15 uL) (turned colorless instantly). Added 10-20 mL of MeCN to decrease turbidity. Purified the solution by Prep-HPLC (A: 0.075% TFA in H₂O, B: ACN) (Note 1: Method A) to give SEQ ID 82 (100.8 mg, 94.7% purity, 23.4% yield for this step; over all yield: 19.8%) obtained as white solid. Analysis was performed using a C18 column with a flow rate of 1 mL/min (Note 2). LCMS Summary: calculated MW: 2058.4, observed MW: 1029.5 [(M+2H)/2], 686.8 [(M+3H)/3].

### EXAMPLE 5: Synthesis of SEQ Id 105

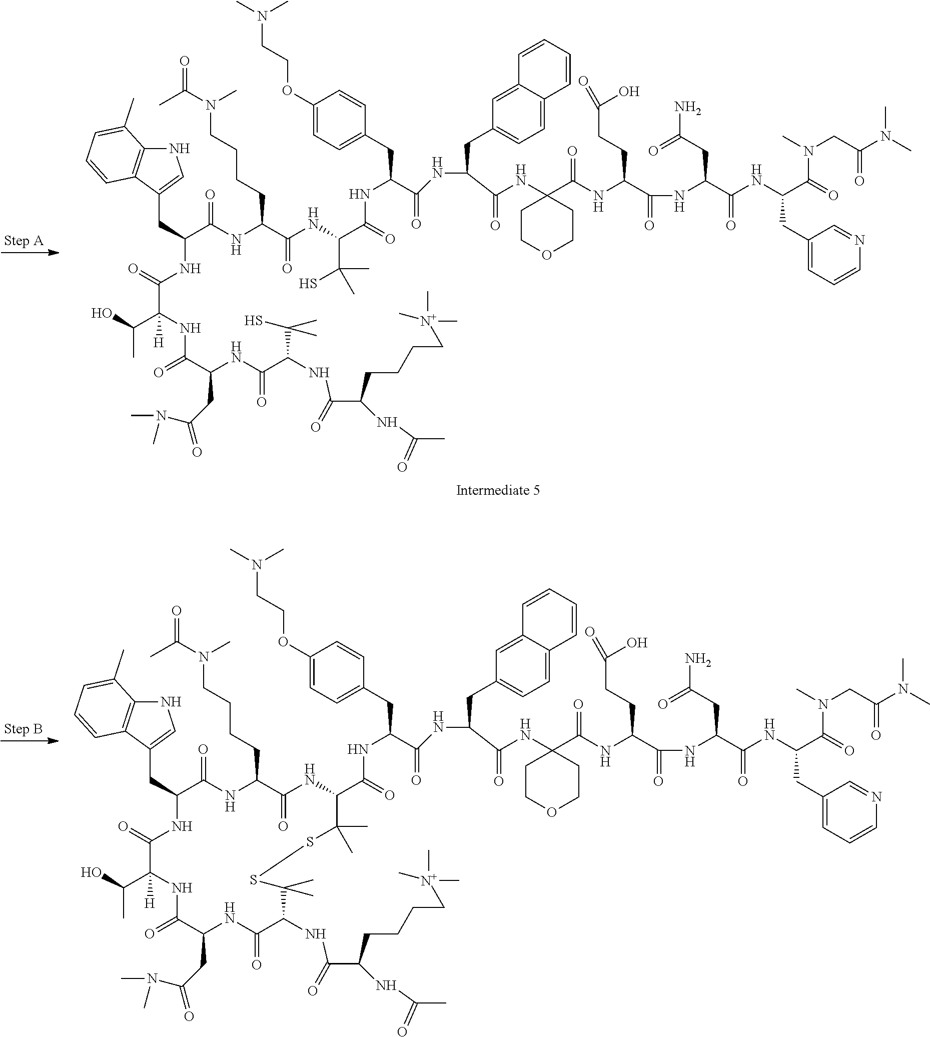

### MeCO-k(Me)₃-Pen(3)—N(N(Me)₂)-T-7MeW—K(NMeAc)-Pen(3)-AEF(N(Me)₂)-2Nal-THP-E-N-3Pya-Sar-CON(Me)2

### Step A Synthesis of Intermediate 5

The peptide was synthesized by standard Solid-phase Peptide Synthesis (SPPS) using Fmoc/t-Bu chemistry. The assembly was performed on a Rink-amide AM resin (440 mol, 100-200Mesh; loading 0.35 mmol/g). First aminoacid was loaded manually using an equimolar solution of Fmoc-Asp-OAll, HOAt and DIC in DMF, at room temperature. Complete acylation was monitored by ninhhydrin test. The resin was then treated with 0.25Eq of Pd Tetrakis, 24 Eq of Phenylsilane in 5 ml of dry DCM under N2 atmosphere for 30 min (process repeated 2 times); washed with DCM, DMF and a solution of 0.5% sodium dimethyldithiocarbamate (0.5%) and DIPEA (0.5%) in DMF. Fmoc-OSu and DIPEA (1:1, 2Eq) in DCM were added and left under stirring at room temperature for 30 minutes. Further peptide elongation was performed manually preactivating the resin with HATU (1.2Eq), DIPEA (2.2Eq) and then adding solution of DIPEA (2.2Eq) and 3Pya-Sar-CON(Me)2 dimer (2.2Eq, TM83) in DMF. The reaction mixture was left under stirring for 1 hr at room temperature. Complete acylation was monitored by test cleavage. The resin was then placed in a MW reaction vessel and the assembly was continued on the Cem Liberty Blue microwave peptide synthesizer (CEM Inc.). During peptide assembly on solid phase, the side chain protecting groups were: tert-butyl for Thr and Glu; trityl for Pen and Asn. D-Lys(Me)₃ at position 3 was coupled manually using DIC-HOBt (3Eq, 1:1:1) using MW irradiation (90° C., 5 min) and complete acylation was monitored by ninhhydrin test.

All the amino acids were dissolved at a 0.4 M concentration in DMF. The acylation reactions were performed for 3 min at 90° C. under MW irradiation with 5 folds excess of activated amino acids over the resin free amino groups. The amino acids were activated with equimolar amounts of 0.5M solution of DIC in DMF and Oxyma solution 1M in DMF. Double acylation reactions were performed for 3Pya15 & 2Nal10. Fmoc deprotections were performed using 20%(V/V) piperidine in DMF. Capping of the free amino group was performed manually using 10eq of acetic anhydride in DMF.

At the end of the assembly the resin was washed with DMF, MeOH, DCM, Et2O. The peptide was cleaved from solid support using 15 ml of TFA solution (v/v) (87.5% TFA, 5% H2O, 2.5% TIPS, 5% Phenol) for approximately 1.5 hours, at room temperature. The resin was then filtered and precipitated in cold MTBE (135 mL). After centrifugation, the peptide pellets were washed with fresh cold diethyl-ether to remove the organic scavengers. The process was repeated twice. Final pellets were dried, re-suspended in H2O and acetonitrile 1:1+0.1% TFA and stirred overnight. Then lyophilized to afford the desired linear intermediate 5 (Y=84%). LCMS anal. calc. For C₁₀₆H155N22023S2+: 2169.66 Da; found; 1085.2 (M+2)²⁺Step B Synthesis of SEQ ID 105

The crude peptide was dissolved in ACN\H2O (5 mg\ml). Saturated Iodine in acetic acid was then added dropwise under stirring until yellow color persisted. Rxn was completed in 30 min (monitored by UPLC-MS). Solid ascorbic acid was added until the solution became clear. After lyophilization the cyclized peptide was purified by reverse-phase HPLC using preparative Waters DeltaPak C4 (200×mm, 300A, 15 μm). Mobile phase A: +0.1% TFA, mobile phase B: Acetonitrile (ACN)+0.1% TFA. The following gradient of eluent B was used: 15% B to 15% B over 5 min, to 30% B over 25 min, flow rate 80 mL/min, wavelength 214 nm. Collected fractions were lyophilized to afford SEQ ID 105 (Y=13.4%). LCMS anal. calc. For C₁₀₆H153N22023S2+: 2167.64 Da; found; 1084.2 (M+2)²⁺

### EXAMPLE 6: Synthesis of SEQ ID 112

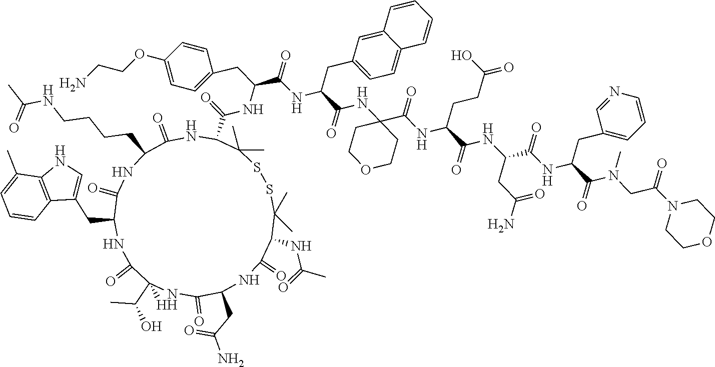 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(=O)N3CCOCC3)CCOCC2)NC1=O]

### MeCO-Pen(3)—N-T-7MeW—K(Ac)-Pen(3)-AEF-2Nal-THIP-E-N-3Pya-Sar-COMor

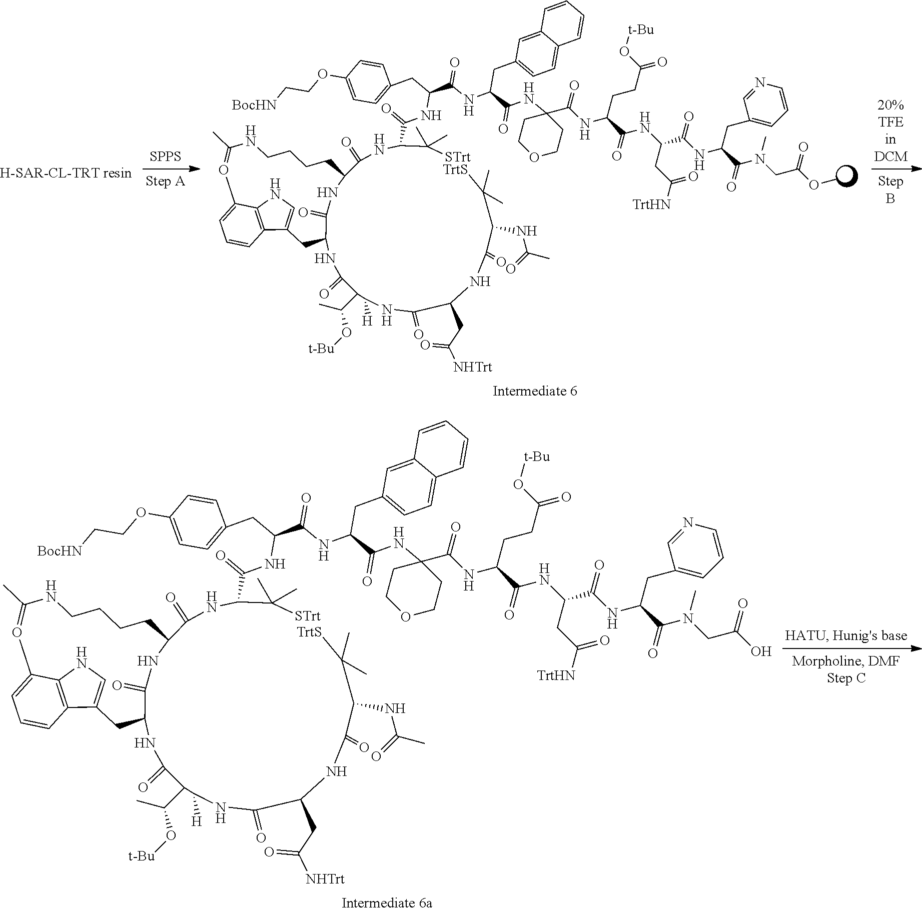

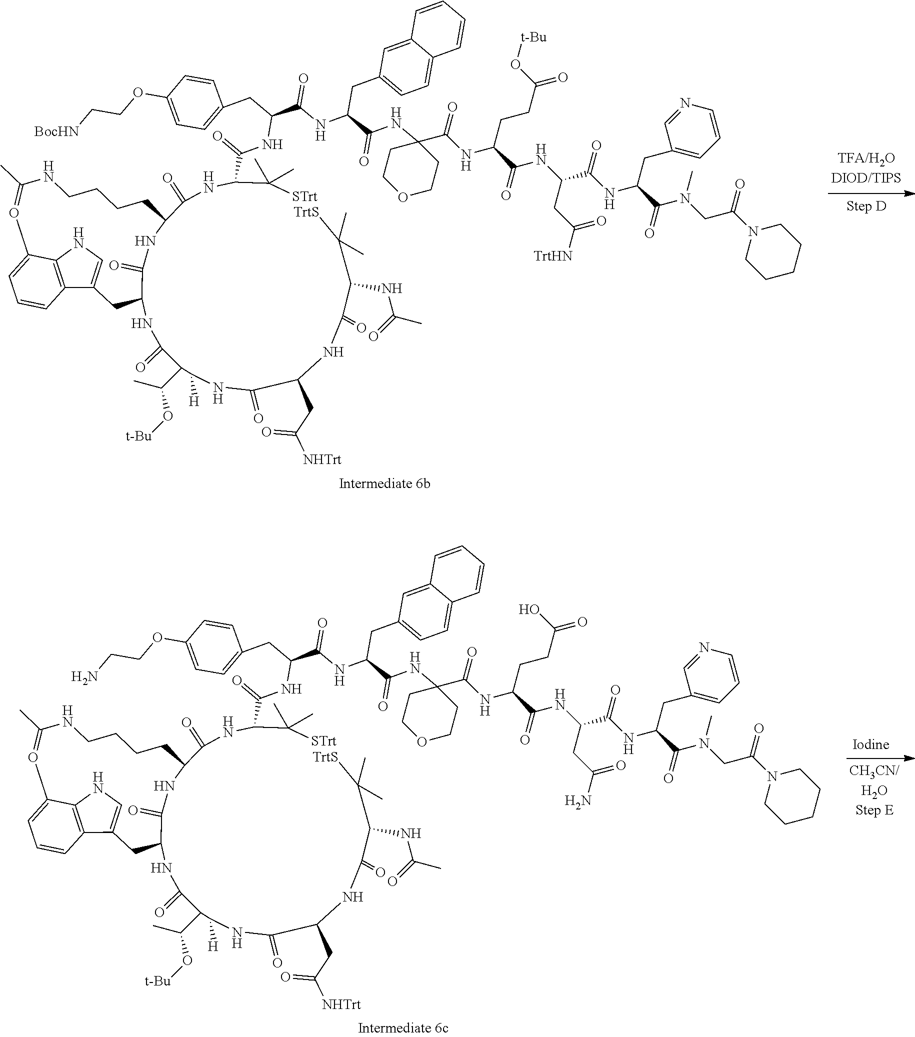

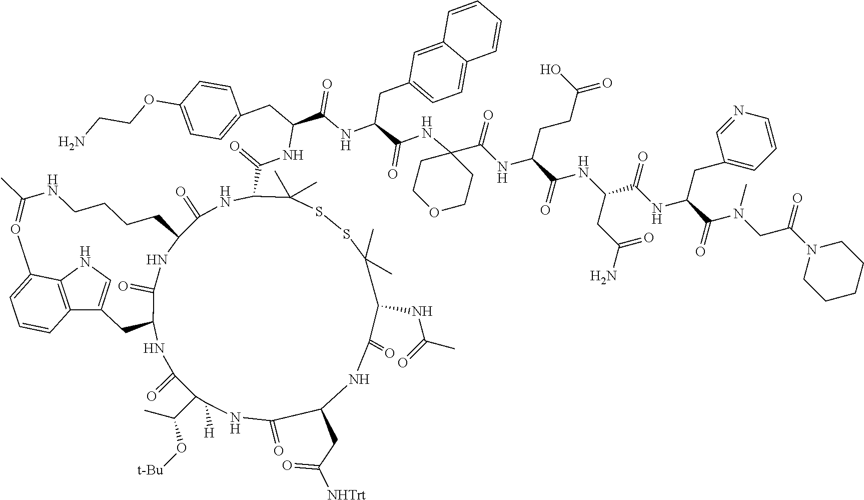

### Step A Synthesis of Intermediate 6

Synthesis was performed using Fmoc-protected amino acids on a solid-phase H-Sar-2-Chlorotrityl resin (AAPPTEC RTG103, 0.3 meq/g, 100-200 mesh) with a CEM Liberty Blue automated microwave peptide synthesizer. Peptide was synthesized on a 0.25 mmol scale. Typical reaction conditions were as follows: Deprotection Conditions: Fmoc deprotection was carried out in 2 stages using 20% piperidine in DMF (10 mL) under microwave conditions (50° C., 2.5 min; 50° C., 5 min). Residue Coupling Conditions: Fmoc-protected amino acid (5 mL of a 0.2 M amino acid stock solution in DMF, 1 mmol) was delivered to the rein, followed by HA TU (2 mL of a 0.45 M stock solution in DMF, 0.9 mmol), and N,N-diisopropylethylamine (1 mL of 2 M solution in NMP, 2 mmol) and allowed to react for 15 min at 50° C. Double couplings were used for 3Pya, THP, and Thr and for residues incorporated after THP and Thr (2Nal and Asn). The peptide was capped with 20% Ac₂O in DMF (10 mL). At the end of the assembly, the peptide resin intermediate 6 was washed with DMF and DCM.

### Step B —Synthesis of Intermediate 6a

Cleavage of 2CTrt resin was carried out by treating the intermediate 6 (0.125 mmol) with a solution of 2,2,2-trifluoroethanol/DCM (2/8, 5 mL) for 1 hr at room temperature. The resin was filtered off and the filtrate was collected. The same operation was repeated three times. The resin was then washed with of 2,2,2-trifluoroethanol/DCM (2/8, 8 mL) three times. The solutions were combined and evaporated to dryness to afford a light yellow solid as intermediate 6a. (Yield: 39%). LCMS anal. calc. For C₁₇₉H₂₀₁N₁₉O₂₅S₂: 3080.4; found: 1552 (M+1+23)²⁺

### Step C-Synthesis of Intermediate 6b

To a solution of intermediate 6a (100 mg, 0.0324 mmol) in DMF (1.0 mL) was added HATU (24.7 mg, 0.0649 mmol), morpholine (5.65 mg, 0.0649 mmol) and Hunig's base (0.0112 mL, 0.75 g/mL, 0.0649 mmol). The reaction mixture was then stirred at room temperature for 30 mins. The reaction mixture was then concentrated to give intermediate 6b as a crude and used for the next step. LCMS anal. calc. for C₁₈₃H₂₀₈N₂₀O₂₅S₂: 3149.5; found: 1587 (M+1+23)²⁺

### Step D —Synthesis of Intermediate 6c

Intermediate 6c was treated with a cocktail solution of TFA/H₂O/DODT/TIPS 92.5/2.5/2.5/2.5 (1 mL) for 30 mins at 42° C. on a CEM Razor cleavage station. The mixture was then concentrated and then added to cold methyl-t-butyl ether in order to precipitate the peptide. After centrifugation, the peptide pellets were washed with fresh cold methyl-t-butyl ether to remove the organic scavengers. The process was repeated twice. Final pellets were dried, re-suspended in H₂O and acetonitrile, then lyophilized to afford the desired protected intermediate 6d as a light yellow solid. (Yield: 78%). LCMS anal. calc. for C₉₄H₁₂₈N₂₀O₂₃S₂: 1968.9; found: 985.4 (M+2)²⁺

### Step E —Synthesis of SEO ID 112

The intermediate 6c from Step D was dissolved in 40% ACN/water (50 mL). Iodine in methanol (0.1 M) was then added drop wise with stirring until yellow color persisted. The reaction was monitored with UPLC-MS. When the reaction was complete, solid ascorbic acid was added until the solution became clear. The solvent mixture was then lyophilized and the resulting material was then dissolved in DMSO and was purified by C18 reverse-phase HPLC (Waters XBridge OBD C18, 50×150 mm, 5 um, 130A&), using as eluents (A) 0.1% TFA in water and (B) 0.1% TFA in acetonitrile, gradient began with 15% B, and changed to 30% B over 25 minutes at a flow rate of 80 ml/min). Fractions containing pure product were collected and then freeze-dried to afford the desired product as a white powder. (Yield: 12.6%). LCMS anal. Calcd for C₉₄H₁₂₆N₂₀O₂₃S₂: 1966.88; found: 985.1 (M+2)²⁺

### Example 7: Synthesis of Seq Id 137

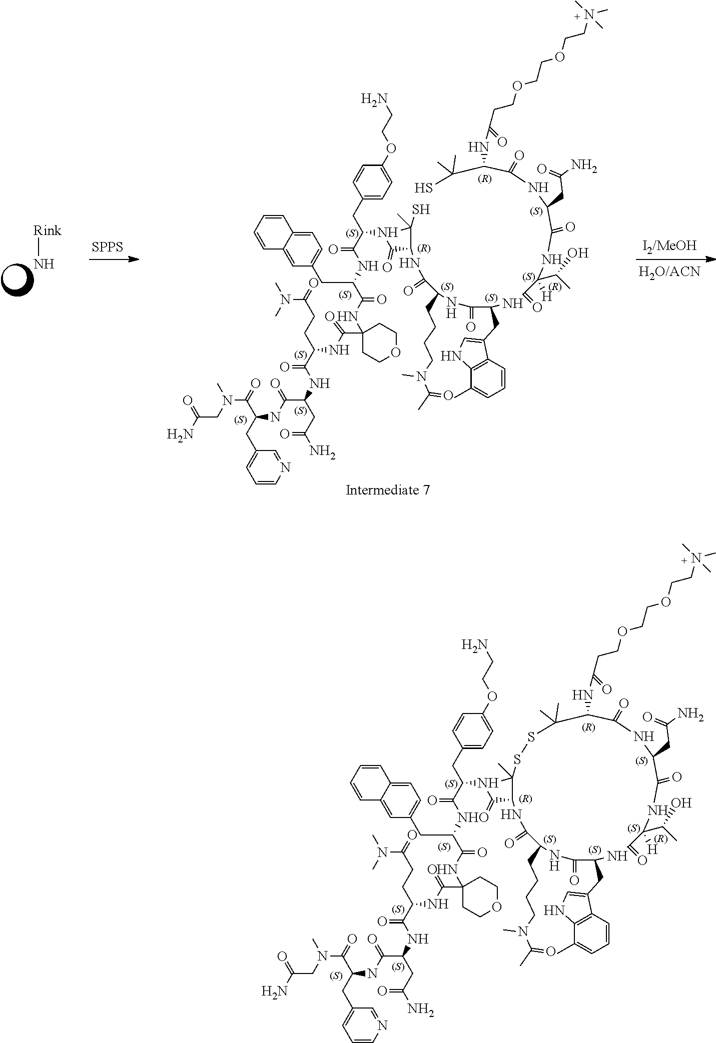

The peptide was synthesized using standard Fmoc chemistry.
1) Add DMF to the vessel containing MBHA Resin (3.0 mmol, 9.67 g, sub: 0.31 mmol/g) and swell for 2 hours.
2) Add 20% piperidine/DMF and mix for 30 min.
3) Drain and then DMF wash 30 sec with 5 times.
4) Add Fmoc-amino acid solution and mix 30 seconds, then add activation buffer, N2 bubbling for about 1-4 hours
5) Add 20% piperidine/DMF and mix for 30 min
6) Repeat step 2 to 5 for next amino acid coupling. The coupling reaction was monitored by ninhydrin or tetrachlor color test, and the resin was washed with DMF for 5 times. The resin was washed with MeOH for 3 times and dried by vacuum

Monitored method:
1. Ninhydrin test: A: 5% ninhydrin/EtOH; B: 80% phenol/EtOH; C: pyridine
2. Tetrachlor color test: A: 2% tetrachlor/DMF; B: 2% aldehyde/DMF 110° C. for 3 min

### Peptide Cleavage:

1) Add cleavage buffer (5.0% DTT/2.5% H₂O/2.5% TIS/90% TFA) 180 mL to the flask containing the side chain protected peptide at room temperature and stir for 3 hrs.
2) Filtered washed with 50 mL TFA. The combined filtrate was precipitated with cold methyl tertbutyl ether (MTBE). The mixture was centrifuged (3000 rpm, 3 min) and decanted. The pellet was washed with MTBE and centrifuged.
3) Lyophilized the residue to give the intermediate 7 (6.0 g, crude)

### Peptide Cyclization and Purification:

Crude peptide intermediate 7 (6.0 g, 2.89 mmol) was dissolved in 20% MeCN/H2O (3000 mL). To a stirred solution of the peptide was added the iodine solution in MeOH (0.1M, 12.0 mL) drop-wise until solution remains yellow. After ˜2h LCMS showed the reaction was complete. Excess iodine was quenched by the addition of 1M Na₂S₂O₃ in water (15 uL) (turned colorless instantly). Added 100-200 mL of MeCN to decrease turbidity. Purified the solution by Prep-HPLC (A: 0.075% TFA in H₂O, B: ACN) (Note 1: Method C) and re-purify by Prep-HPLC (A: 0.5% AcOH in H₂O, B: ACN) (Note 1: Method D), then the product was re-lyophilized with 0.1% TFA mobile phase to give SEQ ID 137 (2448.5 mg, 97.7% purity, 36.7% yield for this step; over all yield: 35.4%) obtained as white solid. Analysis was performed using a C18 column with a flow rate of 1 mL/min (Note 2).

LCMS Summary: calculated MW: 2071.4, observed MW: 1036.0 [(M+2H)/2], 691.1 [(M+3H)/3].

### EXAMPLE 8: Synthesis of SEQ ID 145

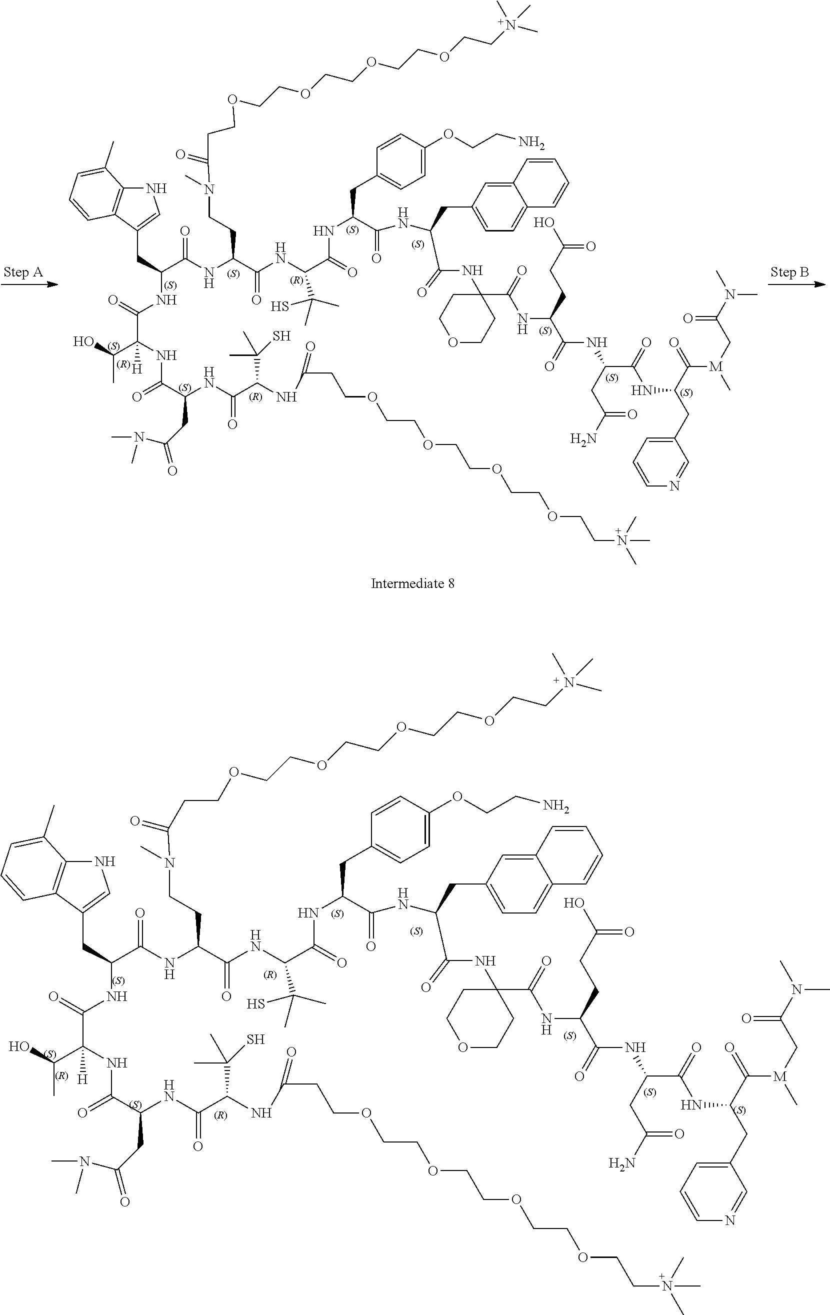

### cPEG5aCO-Pen(3)—N(N(Me)₂)-T-7MeW-Dab(NMecPEG5aCO)-Pen(3)-AEF(N(Me)₂)-2Nal-THP-E-N-3Pya-Sar-CON(Me)2

### Step A Synthesis of Intermediate 8

The peptide was synthesized by standard Solid-phase Peptide Synthesis (SPPS) using Fmoc/t-Bu chemistry. The assembly was performed on a Rink-amide AM resin (220 mol, 100-200Mesh; loading 0.35 mmol/g). First amino acid was loaded manually using an equimolar solution of Fmoc-Asp-OAll, HOAt and DIC in DMF, at room temperature. Complete acylation was monitored by ninhhydrin test. The resin was then treated with 0.25Eq of Pd Tetrakis, 24 Eq of Phenylsilane in 5 ml of dry DCM under N2 atmosphere for 30 min (process repeated 2 times); washed with DCM, DMF and a solution of 0.5% sodium dimethyldithiocarbamate (0.5%) and DIPEA (0.5%) in DMF. Fmoc-OSu and DIPEA (1:1, 2Eq) in DCM were added and left under stirring at room temperature for 30 minutes. Further peptide elongation was performed manually preactivating the resin with HATU (1.2Eq), DIPEA (2.2Eq) and then adding solution of DIPEA (2.2Eq) and 3Pya-Sar-CON(Me)₂ dimer (2.2Eq, TM83) in DMF. The reaction mixture was left under stirring for 1 hr at room temperature. Complete acylation was monitored by test cleavage. The resin was then placed in a MW reaction vessel and the assembly continued on the Cem Liberty Blue microwave peptide synthesizer (CEM Inc.). During peptide assembly on solid phase, the side chain protecting groups were: tert-butyl for Thr and Glu; trityl for Pen and Asn. All the amino acids were dissolved at a 0.4 M concentration in DMF. The acylation reactions were performed for 3 min at 90° C. under MW irradiation with 5 folds excess of activated amino acids over the resin free amino groups. The amino acids were activated with equimolar amounts of 0.5M solution of DIC in DMF and Oxyma solution 1M in DMF. Double acylation reactions were performed for 3Pya15 & 2Nal10. Fmoc deprotections were performed using 20%(V/V) piperidine in DMF. Capping of the free amino group was performed manually using cPEG5aCOOH (2.2 Eq), HATU (2 Eq) and DIPEA(4Eq) at room temperature and complete acylation was monitored by ninhhydrin test.

The resin was then treated with 0.25Eq of Pd Tetrakis, 24 Eq of phenylsilane in 5 ml of DCM Dry under N2 atmosphere for 30 min (process repeated 2 times); washed with DCM, DMF and a solution of 0.5% sodium dimethyldithiocarbamate (0.5%) and DIPEA (0.5%) in DMF. Further side chain derivatization was performed manually with HATU (2Eq), DIPEA (4Eq) and 2.2Eq of cPEG5aCOOH. Reaction was completed after 2 hr (monitored by ninhhydrin test).

At the end of the assembly the resin was washed with DMF, MeOH, DCM, Et2O. The peptide was cleaved from solid support using 30 ml of TFA solution (v/v) (87.5% TFA, 5% H₂O, 2.5% TIPS, 5% Phenol) for approximately 1.5 hours, at room temperature. The resin was then filtered and precipitated in cold MTBE (135 mL). After centrifugation, the peptide pellets were washed with fresh cold diethyl-ether to remove the organic scavengers. The process was repeated twice. Final pellets were dried, re-suspended in H2O and acetonitrile 1:1+0.1% TFA and stirred overnight. Then lyophilized to afford the desired linear intermediate 8 (Y=93%). LCMS anal. calc. For C106H155N22O23S2+: 2438.97 Da Da; found; 1219.4 (M+2)²⁺

### Step B Synthesis of SEQ ID 145

The crude peptide was dissolved in ACN\H2O (5 mg\ml). Saturated Iodine in acetic acid was then added dropwise under stirring until yellow color persisted. Rxn was completed in 30 min (monitored by UPLC-MS). Solid ascorbic acid was added until the solution became clear. After lyophilization the cyclized peptide was purified by reverse-phase HPLC using preparative Waters DeltaPak C4 (200×mm, 300A, 15 μm). Mobile phase A: +0.1% TFA, mobile phase B: Acetonitrile (ACN)+0.1% TFA. The following gradient of eluent B was used: 15% B to 15% B over 5 min, to 30% B over 25 min, flow rate 80 mL/min, wavelength 214 nm. Collected fractions were lyophilized to afford SEQ ID 145 (Y=10%) LCMS anal. calc. For C₁₁₇H178N22030S22+: 2436.95 Da; found; 1218.5 (M+2)²⁺

### Example 9:Synthesis of Seq Id 164

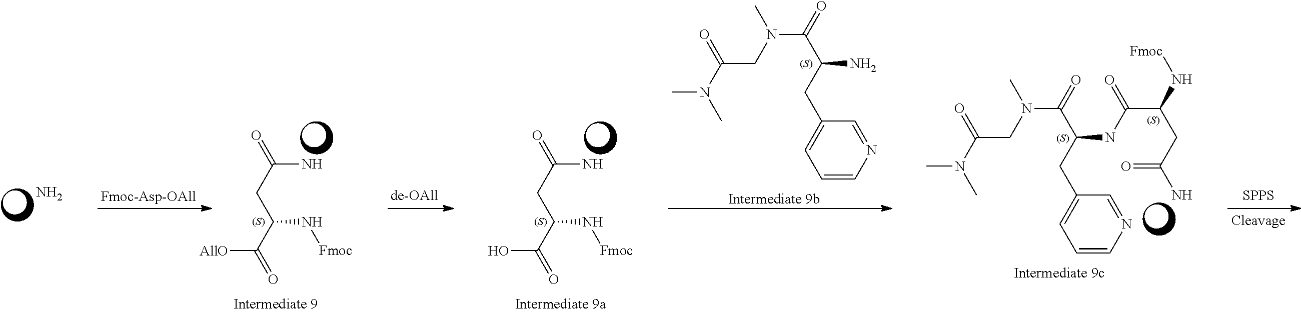

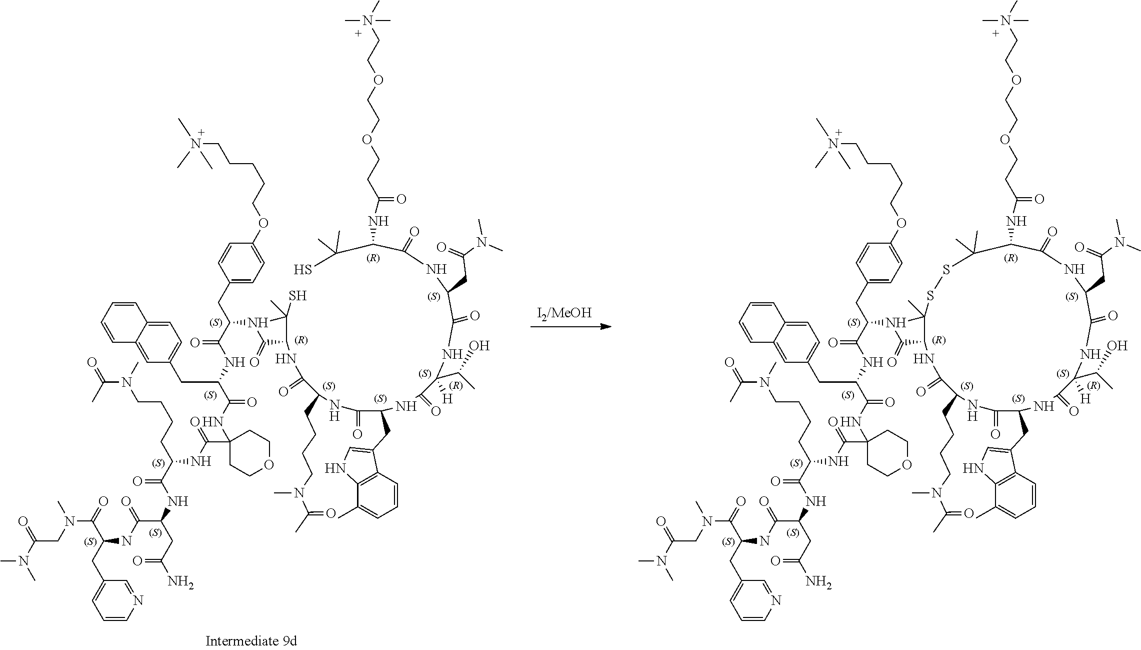

The peptide was synthesized using standard Fmoc chemistry.
1) Add DMF to the vessel containing Rink Amide MBHA resin (15.0 mmol, 50 g, sub: 0.3 mmol/g) and swell for 2 hours.
2) Add 20% piperidine/DMF and mix for 30 min.
3) Drain and then DMF wash 30 sec with 5 times.
4) Add Fmoc-amino acid solution and mix 30 seconds, then add activation buffer, N₂ bubbling for about 1-5 hours
5) Add 20% piperidine/DMF and mix for 30 min
6) Repeat step 2 to 5 for next amino acid coupling. The coupling reaction was monitored by ninhydrin or tetrachlor color test, and the resin was washed with DMF for 5 times. The resin was washed with MeOH for 3 times and dried by vacuum.

Monitored method:
1. Ninhydrin test: A: 5% ninhydrin/EtOH; B: 80% phenol/EtOH; C: pyridine
2. Tetrachlor color test: A: 2% tetrachlor/DMF; B: 2% aldehyde/DMF 110° C. for 3 min

### Peptide Cleavage:

1) Add cleavage buffer (5.0% DTT/2.5% H₂O/2.5% TIS/90% TFA) 1000 mL to the flask containing the side chain protected peptide at room temperature and stir for 3 hrs.
2) Filtered washed with 100 mL TFA. The combined filtrate was precipitated with cold methyl tertbutyl ether (MTBE). The mixture was centrifuged (3000 rpm, 3 min) and decanted. The pellet was washed with MTBE and centrifuged.
3) Lyophilized the residue to give the intermediate 9d (30.0 g, crude)

### Peptide Cyclization and Purification:

The reaction was performed for 3 batches in parallel. Crude peptide intermediate 9d (10.0 g, 4.4 mmol) was dissolved in 20% MeCN/H₂O (5.0 L). To a stirred solution of the peptide was added the iodine solution in MeOH (0.1M, 15 mL) drop-wise until solution remains yellow. After ˜2h LCMS showed the reaction was complete. Excess iodine was quenched by the addition of 1M Na₂S₂O₃ in water (15 uL) (turned colorless instantly). Added 100-200 mL of MeCN to decrease turbidity. Purified the solution by Prep-HPLC (A: 0.075% TFA in H₂O, B: ACN) (Note 1: Method E) and re-purified by Prep-HPLC (A: 0.5% AcOH in H₂O, B: ACN) (Note 1: Method F) to give SEQ ID 164 (3.5 g, 92.2% purity, 9.9% yield for this step; over all yield: 8.7%) obtained as white solid. Analysis was performed using a C18 column with a flow rate of 1 mL/min (Note 2). LCMS Summary: calculated MW: 2268.8, observed MW: 1133.8 [(M+2H)/2], 756.2 [(M+3H)/3].

### Example 10: Synthesis of Seq Id 181

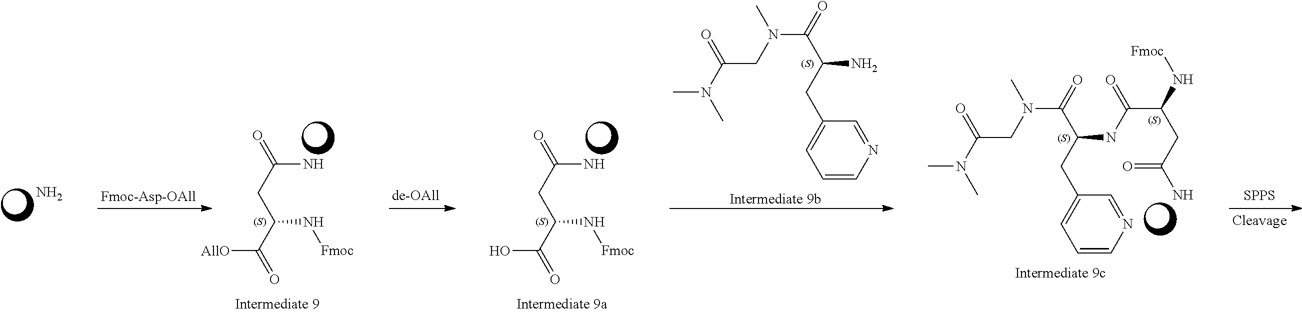

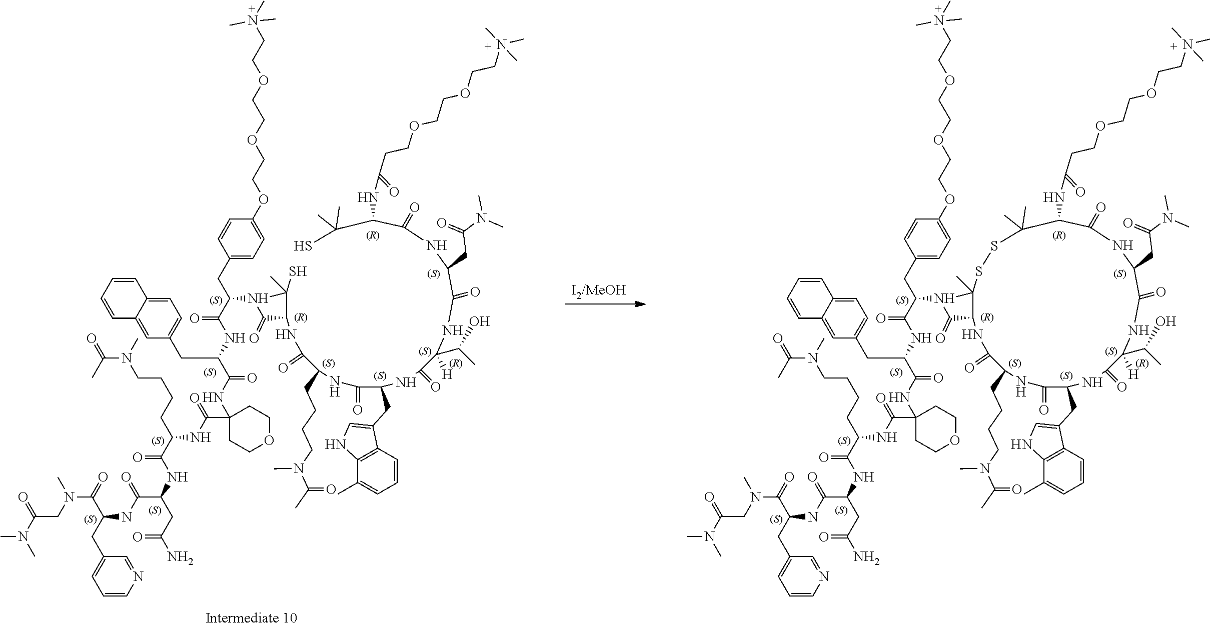

The peptide was synthesized using standard Fmoc chemistry.
1) Add DMF to the vessel containing Rink Amide MBHA resin (15.0 mmol, 43 g, sub: 0.35 mmol/g) and swell for 2 hours.
2) Add 20% piperidine/DMF and mix for 30 min.
3) Drain and then DMF wash 30 sec with 5 times.
4) Add Fmoc-amino acid solution and mix 30 seconds, then add activation buffer, N₂ bubbling for about 1-4 hours
5) Add 20% piperidine/DMF and mix for 30 min
6) Repeat step 2 to 5 for next amino acid coupling. The coupling reaction was monitored by ninhydrin or tetrachlor color test, and the resin was washed with DMF for 5 times. The resin was washed with MeOH for 3 times and dried by vacuum.

Monitored method:
1. Ninhydrin test: A: 5% ninhydrin/EtOH; B: 80% phenol/EtOH; C: pyridine
2. Tetrachlor color test: A: 2% tetrachlor/DMF; B: 2% aldehyde/DMF 110° C. for 3 min

### Peptide Cleavage:

1) Add cleavage buffer (5.0% DTT/2.5% H₂O/2.5% TIS/90% TFA) 1000 mL to the flask containing the side chain protected peptide at room temperature and stir for 3 hrs.
2) Filtered washed with 100 mL TFA. The combined filtrate was precipitated with cold methyl tertbutyl ether (MTBE). The mixture was centrifuged (3000 rpm, 3 min) and decanted. The pellet was washed with MTBE and centrifuged.
3) Lyophilized the residue to give the intermediate 10 (30.0 g, crude)

### Peptide Cyclization and Purification:

The reaction was performed for 3 batches in parallel. Crude peptide intermediate 10 (10.0 g, 4.3 mmol) was dissolved in 20% MeCN/H2O (5.0 L). To a stirred solution of the peptide was added the iodine solution in MeOH (0.1M, 17 mL) drop-wise until solution remains yellow. After ˜2h LCMS showed the reaction was complete. Excess iodine was quenched by the addition of 1M Na₂S₂O₃ in water (15 uL) (turned colorless instantly). Added 100-200 mL of MeCN to decrease turbidity. Purified the solution by Prep-HPLC (A: 0.075% TFA in H₂O, B: ACN) (Note 1: Method E) and re-purified by Prep-HPLC (A: 0.5% AcOH in H₂O, B: ACN) (Note 1: Method F) to give SEQ ID 181 (4.5 g, 96.6% purity, 14.5% yield for this step, over all yield: 12.5% yield) obtained as white solid.

Analysis was performed using a C18 column with a flow rate of 1 mL/min (Note 2). LCMS Summary: calculated MW: 2314.8, observed MW: 1157.3 [(M+2H)/2], 771.7 [(M+3H)/3].

### Example 11:Synthesis of Seq Id 217

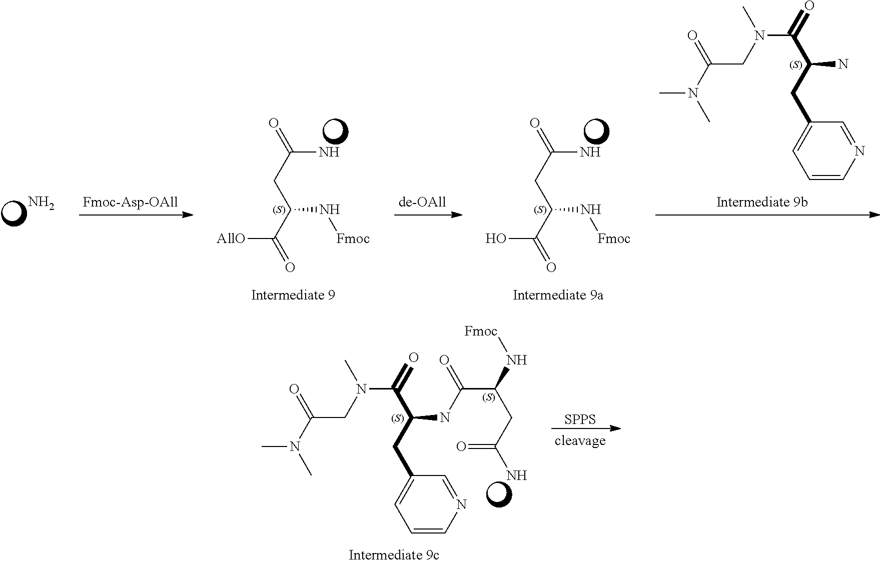

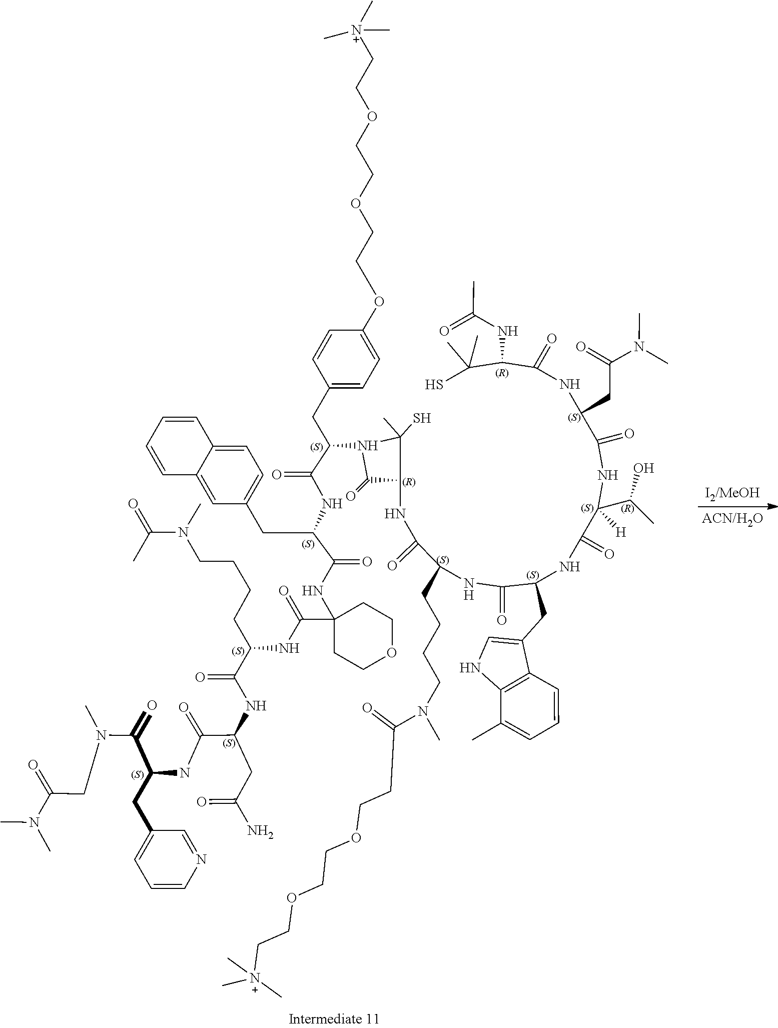

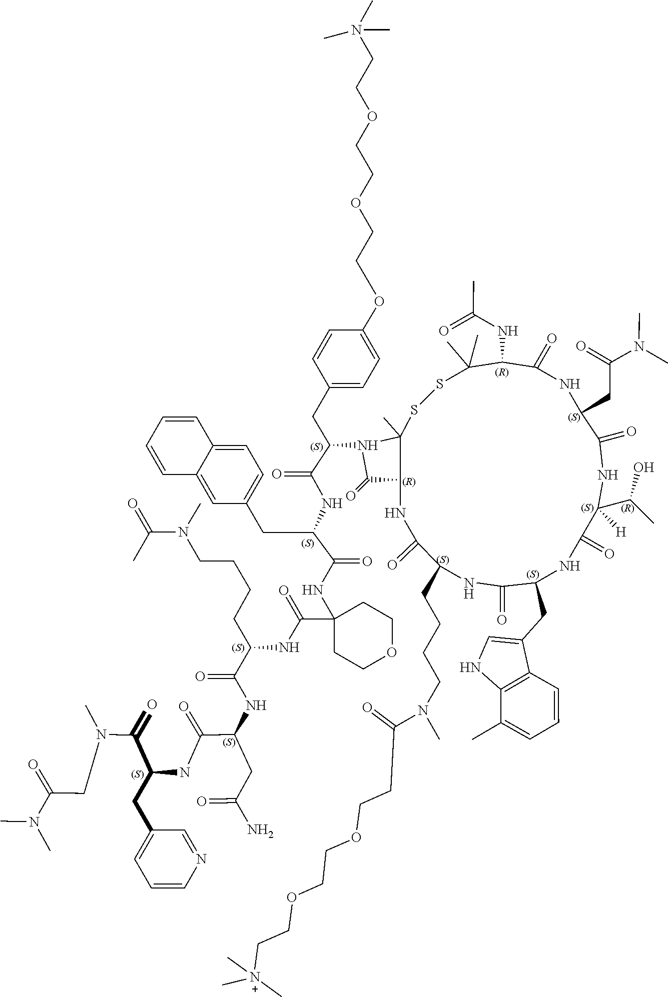

The peptide was synthesized using standard Fmoc chemistry.
1) Add DMF to the vessel containing Rink Amide MBHA resin (0.50 mmol, 1.78 g, sub: 0.28 mmol/g) and swell for 2 hours.
2) Add 20% piperidine/DMF and mix for 30 min.
3) Drain and then DMF wash 30 sec with 5 times.
4) Add Fmoc-amino acid solution and mix 30 seconds, then add activation buffer, N₂ bubbling for about 1-4 hours
5) Add 20% piperidine/DMF and mix for 30 min
6) Repeat step 2 to 5 for next amino acid coupling. The coupling reaction was monitored by ninhydrin or tetrachlor color test, and the resin was washed with DMF for 5 times. The resin was washed with MeOH for 3 times and dried by vacuum.

Monitored method:
1. Ninhydrin test: A: 5% ninhydrin/EtOH; B: 80% phenol/EtOH; C: pyridine
2. Tetrachlor color test: A: 2% tetrachlor/DMF; B: 2% aldehyde/DMF 110° C. for 3 min

### Peptide Cleavage:

4) Add cleavage buffer (5.0% DTT/2.5% H₂O/2.5% TIS/90% TFA) 35 mL to the flask containing the side chain protected peptide at room temperature and stir for 3 hrs.
5) Filtered washed with 5 mL TFA. The combined filtrate was precipitated with cold methyl tertbutyl ether (MTBE). The mixture was centrifuged (3000 rpm, 3 min) and decanted. The pellet was washed with MTBE and centrifuged.
6) Lyophilized the residue to give the intermediate 11 (1.0 g, crude)

### Peptide Cyclization and Purification:

Crude peptide intermediate 11 (1.0 g, 0.43 mmol) was dissolved in 20% MeCN/H2O (500 mL). To a stirred solution of the peptide was added the iodine solution in MeOH (0.1M, 6.0 mL) drop-wise until solution remains yellow. After ˜2h LCMS showed the reaction was complete. Excess iodine was quenched by the addition of 1M Na₂S₂O₃ in water (turned colorless instantly). Added 10 mL of MeCN to decrease turbidity. Purified the solution by Prep-HPLC (A: 0.075% TFA in H₂O, B: ACN) (Note 1: Method B) to give SEQ ID 217 (139.8 mg, 98.9% purity, 12.0% yield for this step, over all yield: 10.4% yield) obtained as white solid. Analysis was performed using a C18 column with a flow rate of 1 mL/min (Note 2). LCMS Summary: calculated MW: 2314.8, observed MW: 1157.2 [(M+2H)/2], 771.6 [(M+3H)/3].

### Biological Assays

### IL23R Reporter Assay

Compounds were serially diluted in 100% (v/v) DMSO) and plated using an Echo acoustic dispenser (Labcyte) into 1536-well non-treated black assay plates (Corning #9146). 3 μL of HEK293 cells containing IL-23R, IL-12RP1 and a firefly luciferase reporter gene driven by a STAT-inducible promoter (Promega) were added to the plates (4000 cells/well), followed by 3 μL of 10 ng/mL IL-23 (equivalent to EC₉₀ concentration). After 5 h at 37° C., 5% CO₂, 95% relative humidity, cells were placed at 20° C. and treated with BioGlo reagent (Promega) according to the Manufacturer's instructions. Luminescence was measured on a Pherastar FSX (BMG LabTech). Data were normalized to IL-23 treatment (0% inhibition) and 30 μM of control inhibitor (100% inhibition), and IC₅₀ values were determined using a 4-parameter Hill equation. Data for exemplary compounds are shown in Tables 3a and 3b.

TABLE 3
IL-23R Reporter Assay Data

| | IL23R |
|---|---|
| | Reporter |
| SEQ | Assay |
| ID | (micromolar) |
| 1 | ~6.29 |
| 2 | 1.05 |
| 3 | 2 |
| 4 | 0.29 |
| 5 | 0.053 |
| 6 | 0.064 |
| 7 | 0.51 |
| 8 | 0.089 |
| 9 | 0.053 |
| 10 | 0.23 |
| 11 | >16.61 |
| 12 | >16.61 |
| 13 | 0.16 |
| 14 | 0.14 |
| 15 | 0.21 |
| 16 | 0.024 |
| 17 | 0.038 |
| 18 | 0.043 |
| 19 | 0.11 |
| 20 | 0.3 |
| 21 | 0.13 |
| 22 | 0.16 |
| 23 | 0.15 |
| 24 | 0.18 |
| 25 | 0.048 |
| 26 | 0.11 |
| 27 | 0.39 |
| 28 | 0.71 |
| 29 | 0.53 |
| 30 | 0.0035 |
| 31 | 0.32 |
| 32 | 0.016 |
| 33 | 0.5 |
| 34 | 0.33 |
| 35 | 0.0034 |
| 36 | 0.94 |
| 37 | 0.0053 |
| 38 | 0.0054 |
| 39 | 0.0055 |
| 40 | 0.0068 |
| 41 | 0.0045 |
| 42 | 0.0047 |
| 43 | 0.0068 |
| 44 | 0.0068 |
| 45 | 0.0071 |
| 46 | 0.0032 |
| 47 | 0.0039 |
| 48 | 0.73 |
| 49 | 0.0044 |
| 50 | 0.0036 |
| 51 | 0.026 |
| 52 | 0.0046 |
| 53 | 0.0039 |
| 54 | 0.0053 |
| 55 | 0.0025 |
| 56 | 0.0057 |
| 57 | 0.0029 |
| 58 | 0.0043 |
| 59 | 0.0047 |
| 60 | 0.0035 |
| 61 | 0.0033 |
| 62 | 0.0032 |
| 63 | 0.025 |
| 64 | 0.0049 |
| 65 | 0.0049 |
| 66 | 0.0028 |
| 67 | 0.0023 |
| 68 | 0.0029 |
| 69 | 0.0019 |
| 70 | 0.0035 |
| 71 | 0.0021 |
| 72 | 0.0025 |
| 73 | 0.0019 |
| 74 | 0.0024 |
| 75 | 0.0029 |
| 76 | 0.0032 |
| 77 | 0.0038 |
| 78 | 0.0039 |
| 79 | 0.0039 |
| 80 | 0.0043 |
| 81 | 0.0029 |
| 82 | 0.012 |
| 83 | 0.0033 |
| 84 | 0.0035 |
| 85 | 0.0037 |
| 86 | 0.0047 |
| 87 | 0.0042 |
| 88 | 0.0039 |
| 89 | 0.0027 |
| 90 | 0.0037 |
| 91 | 0.0028 |
| 92 | 0.0035 |
| 93 | 0.0036 |
| 94 | 0.0039 |
| 95 | 0.0048 |
| 96 | 0.0046 |
| 97 | 0.0058 |
| 98 | 0.0046 |
| 99 | 0.0039 |
| 100 | 0.0093 |
| 101 | 0.0052 |
| 102 | 0.0055 |
| 103 | 0.0054 |
| 104 | 0.0048 |
| 105 | 0.0062 |
| 106 | 0.0065 |
| 107 | 0.0045 |
| 108 | 0.0046 |
| 109 | 0.0062 |
| 110 | 0.0044 |
| 111 | 0.004 |
| 112 | 0.0053 |
| 113 | 0.0038 |
| 114 | 0.028 |
| 115 | 0.0026 |
| 116 | 0.0017 |
| 117 | 0.0022 |
| 118 | 0.0021 |
| 119 | 0.0063 |
| 120 | 0.0023 |
| 121 | 0.013 |
| 122 | 0.0021 |
| 123 | 0.0019 |
| 124 | 0.0021 |
| 125 | 0.002 |
| 126 | 0.0013 |
| 127 | 0.0026 |
| 128 | 0.00079 |
| 129 | 0.0016 |
| 130 | 0.0034 |
| 131 | 0.0023 |
| 132 | 0.0019 |
| 133 | 0.0019 |
| 134 | 0.0021 |
| 135 | 0.0037 |
| 136 | 0.0026 |
| 137 | 0.0036 |
| 138 | 0.0027 |
| 139 | 0.0034 |
| 140 | 0.003 |
| 141 | 0.0057 |
| 142 | 0.0066 |
| 143 | 0.0065 |
| 144 | 0.0025 |
| 145 | 0.0057 |
| 146 | 0.0038 |
| 147 | 0.0038 |
| 148 | 0.0026 |
| 149 | 0.0021 |
| 150 | 0.0033 |
| 151 | 0.0066 |
| 152 | 0.0016 |
| 153 | 0.27 |
| 154 | 0.0024 |
| 155 | 0.0022 |
| 156 | 0.002 |
| 157 | 0.0028 |
| 158 | 0.0039 |
| 159 | 0.0018 |
| 160 | 0.0024 |
| 161 | 0.004 |
| 162 | 0.0033 |
| 163 | 0.0032 |
| 164 | 0.0024 |
| 165 | 0.0043 |
| 166 | 0.003 |
| 167 | 0.0017 |
| 168 | 0.0025 |
| 169 | 0.0023 |
| 170 | 0.004 |
| 171 | 0.0029 |
| 172 | 0.0065 |
| 173 | 0.0031 |
| 174 | 0.0036 |
| 175 | 0.0063 |
| 176 | 0.0039 |
| 177 | 0.002 |
| 178 | 0.002 |
| 179 | 0.003 |
| 180 | 0.0029 |
| 181 | 0.002 |
| 182 | 0.0017 |
| 183 | 0.0018 |
| 184 | 0.0034 |
| 185 | 0.0019 |
| 186 | 0.0017 |
| 187 | 0.086 |
| 188 | 0.0023 |
| 189 | 0.005 |
| 190 | 0.0022 |
| 191 | 0.0025 |
| 192 | 0.0041 |
| 193 | 0.04 |
| 194 | 0.0035 |
| 195 | 0.0029 |
| 196 | 0.0039 |
| 197 | 0.0029 |
| 198 | 0.0031 |
| 199 | 0.003 |
| 200 | 0.0028 |
| 201 | 0.0022 |
| 202 | 0.0035 |
| 203 | 0.0016 |
| 204 | 0.0046 |
| 205 | 0.004 |
| 206 | 0.0026 |
| 207 | 0.0032 |
| 208 | 0.0032 |
| 209 | 0.0023 |
| 210 | 0.0022 |
| 211 | 0.0023 |
| 212 | 0.0031 |
| 213 | 0.0051 |
| 214 | 0.0059 |
| 215 | 0.0023 |
| 216 | 0.0029 |
| 217 | 0.0038 |
| 218 | 0.0038 |
| 219 | 0.0027 |
| 220 | 0.0014 |
| 221 | 0.0018 |
| 222 | 0.0033 |
| 223 | 0.0022 |
| 224 | 0.003 |
| 225 | 0.0041 |
| 226 | 0.0028 |
| 227 | 0.0039 |
| 228 | 0.0033 |
| 229 | 0.019 |
| 230 | 0.0038 |
| 231 | 0.0029 |
| 232 | 0.0034 |
| 233 | 0.003 |
| 234 | 0.0033 |
| 235 | 0.0033 |
| 236 | 0.003 |
| 237 | 0.0027 |
| 238 | 0.0035 |
| 239 | 0.0027 |
| 240 | 0.003 |
| 241 | 0.0024 |
| 242 | 0.025 |
| 243 | 0.0039 |
| 244 | 0.0041 |
| 245 | 0.042 |
| 246 | 0.0065 |
| 247 | ND |
| 248 | ND |
| 249 | 0.0036 |
| 250 | 0.0069 |
| 251 | 0.0029 |
| 252 | 0.0046 |
| 253 | 0.04 |
| 254 | 0.0011 |
| 255 | 0.022 |
| 256 | 0.12 |
| 257 | 0.0082 |
| 258 | 0.0069 |
| 259 | 0.27 |
| 260 | 0.0054 |
| 261 | 0.002 |
| 262 | 0.0036 |
| 263 | 0.005 |
| 264 | 0.0022 |
| 265 | 0.0064 |
| 266 | 0.0024 |
| 267 | 0.0042 |
| 268 | 0.11 |
| 269 | 0.0014 |
| 270 | 0.0022 |
| 271 | 0.0022 |
| 272 | 0.0022 |
| 273 | 0.0026 |
| 274 | ND |
| 275 | ND |
| 276 | 0.055 |
| 277 | 0.0029 |
| 278 | 0.0043 |
| 279 | 0.0023 |
| 280 | 0.0027 |
| 281 | 0.0016 |
| 282 | 0.0013 |
| 283 | 0.0036 |
| 284 | 0.0033 |
| 285 | 0.0038 |
| 286 | 0.0032 |
| 287 | 0.024 |
| 288 | 0.024 |
| 289 | 0.079 |
| 290 | 0.12 |
| 291 | 0.61 |
| 292 | 0.003 |
| 293 | 0.0032 |
| 294 | 0.0012 |
| 295 | 0.0024 |
| 296 | 0.0033 |
| 297 | 0.0018 |
| 298 | 0.0017 |
| 299 | 0.0019 |
| 300 | 0.0021 |
| 301 | 0.00073 |
| 302 | 0.0013 |
| 303 | 0.0036 |
| 304 | 0.0014 |
| 305 | 0.0024 |
| 306 | 0.0011 |
| 307 | 0.017 |
| 308 | 0.002 |
| 309 | 0.0011 |
| 310 | 0.0022 |
| 311 | 0.0013 |
| 312 | 0.0011 |
| 313 | 0.0042 |
| 314 | 0.007 |
| 315 | 0.0085 |
| 316 | 0.0013 |
| 317 | 0.0013 |
| 318 | 0.00089 |
| 319 | 0.0018 |
| 320 | 0.00095 |
| 321 | 0.0013 |
| 322 | 0.0048 |
| 323 | 0.094 |
| 324 | 0.0087 |
| 325 | 0.2 |
| 326 | 0.00085 |
| 327 | 0.0012 |
| 328 | 0.0012 |
| 329 | 0.0019 |
| 330 | 0.0013 |
| 331 | 0.0012 |
| 332 | 0.0012 |
| 333 | 0.0018 |
| 334 | 0.0011 |
| 335 | 0.0012 |
| 336 | 0.0015 |
| 337 | 0.0019 |
| 338 | 0.0016 |
| 339 | 0.0019 |
| 340 | 0.0028 |
| 341 | 0.00097 |
| 342 | 0.0017 |
| 343 | 0.002 |
| 344 | 0.0066 |
| 345 | 0.0025 |
| 346 | 0.0018 |
| 347 | 0.12 |
| 348 | 0.15 |
| 349 | 0.22 |
| 350 | 0.19 |
| 351 | 0.15 |
| 352 | 0.19 |
| 353 | 0.14 |
| 354 | 0.33 |
| 355 | 0.35 |
| 356 | 0.28 |
| 357 | 0.31 |
| 358 | 0.0053 |
| 359 | 0.014 |
| 360 | 0.0084 |
| 361 | 0.012 |
| 362 | 0.0022 |
| 363 | 0.0013 |
| 364 | 0.0012 |
| 365 | 0.0013 |
| 366 | 0.0048 |
| 367 | 0.0091 |
| 368 | 0.15 |
| 369 | 0.18 |
| 370 | 0.061 |
| 371 | 0.0055 |
| 372 | 0.02 |
| 373 | 0.087 |
| 374 | 0.14 |
| 375 | 0.24 |
| 376 | 0.14 |
| 377 | 0.097 |
| 378 | 0.0062 |
| 379 | 0.012 |
| 380 | 0.074 |
| 381 | 0.079 |
| 382 | 0.23 |
| 383 | 0.018 |
| 384 | 0.0014 |
| 385 | 0.1 |
| 386 | 0.046 |
| 387 | 0.012 |
| 388 | 0.034 |
| 389 | 0.39 |
| 390 | 0.0012 |
| 391 | 0.0015 |
| 392 | ND |
| 393 | 0.0019 |
| 394 | ND |
| 395 | 0.091 |
| 396 | 0.88 |
| 397 | ND |
| 398 | 0.44 |
| 399 | 0.015 |
| 400 | 0.29 |
| 401 | 0.0059 |
| 402 | 0.013 |
| 403 | 0.0045 |
| 404 | 0.031 |
| 405 | 0.0019 |
| 406 | 0.0021 |
| 407 | 0.0059 |
| 408 | 0.0047 |
| 409 | 0.047 |
| 410 | 0.32 |
| 411 | 0.0021 |
| 412 | 0.0021 |
| 413 | 0.0017 |
| 414 | 0.0027 |
| 415 | 0.0044 |
| 416 | 0.0012 |
| 417 | 0.0014 |
| 418 | 0.0012 |
| 419 | 0.003 |
| 420 | 0.0023 |
| 421 | 0.0012 |
| 422 | 0.0018 |
| 423 | 0.0011 |
| 424 | 0.0026 |
| 425 | 0.0017 |
| 426 | 0.0016 |
| 427 | 0.0039 |
| 428 | 0.0035 |
| 429 | 0.0017 |
| 430 | 0.0026 |
| 431 | 0.0023 |
| 432 | 0.0036 |
| 433 | 0.0036 |
| 434 | 0.0031 |
| 435 | 0.0022 |
| 436 | 0.0015 |
| 437 | 0.0012 |
| 438 | 0.0034 |
| 439 | 0.0025 |
| 440 | 0.002 |
| 441 | 0.0027 |
| 442 | 0.0029 |
| 443 | 0.0021 |
| 444 | 0.0029 |
| 445 | 0.0017 |
| 446 | 0.0024 |
| 447 | 0.002 |
| 448 | 0.002 |
| 449 | 0.0018 |
| 450 | 0.0017 |
| 451 | 0.0015 |
| 452 | 0.0022 |
| 453 | 0.0023 |
| 454 | 0.0021 |
| 455 | 0.0021 |
| 456 | 0.0015 |
| 457 | 0.0013 |
| 458 | 0.0026 |
| 459 | 0.0018 |
| 460 | 0.0018 |
| 461 | 0.0014 |
| 462 | 0.0013 |
| 463 | 0.009 |
| 464 | 0.03 |
| 465 | 0.017 |
| 466 | 0.0018 |
| 467 | 0.0012 |
| 468 | 0.025 |
| 469 | 0.0021 |
| 470 | 0.0012 |
| 471 | 0.00069 |
| 472 | 0.0016 |
| 473 | 0.012 |
| 474 | 0.0014 |
| 475 | 1.54 |
| 476 | >2.59 |
| 477 | 0.0061 |
| 478 | 0.0023 |
| 479 | 0.0034 |
| 480 | 0.0036 |
| 481 | 0.0035 |
| 482 | 0.0039 |
| 483 | 0.0038 |
| 484 | 0.0031 |
| 485 | 0.0036 |
| 486 | 0.0044 |
| 487 | 0.0034 |
| 488 | 0.0057 |
| 489 | 0.0037 |
| 490 | 0.0033 |
| 491 | 0.0083 |
| 492 | 0.0064 |
| 493 | 0.017 |
| 494 | 0.012 |
| 495 | 0.0036 |
| 496 | 0.006 |
| 497 | 0.08 |
| 498 | 0.027 |
| 499 | >1.63 |
| 500 | >1.19 |
| 501 | 0.0046 |
| 502 | 0.0038 |
| 503 | 0.0084 |
| 504 | 0.0086 |
| 505 | 0.0036 |
| 506 | 0.0041 |
| 507 | 0.003 |
| 508 | 0.0029 |
| 509 | 0.032 |
| 510 | >1.22 |
| 511 | >16.61 |
| 512 | >7.35 |
| 513 | 0.0053 |
| 514 | 0.0047 |
| 515 | 0.0038 |
| 516 | 0.05 |
| 517 | 0.051 |
| 518 | 0.018 |
| 519 | 0.0018 |
| 520 | 0.0014 |
| 521 | 0.0014 |
| 522 | 0.0016 |
| 523 | 0.0052 |
| 524 | 0.054 |
| 525 | 0.051 |
| 526 | 0.0061 |
| 527 | 0.0037 |
| 528 | 0.0038 |
| 529 | 0.0033 |
| 530 | 0.0029 |
| 531 | 0.0037 |
| 532 | 0.0009 |
| 533 | 0.0016 |
| 534 | 0.0021 |
| 535 | 0.0022 |
| 536 | 0.0029 |
| 537 | 0.0029 |
| 538 | 0.0035 |
| 539 | 0.003 |
| 540 | 0.0039 |
| 541 | 0.00097 |
| 542 | 0.0019 |
| 543 | 0.002 |
| 544 | 0.002 |
| 545 | 0.00083 |
| 546 | 0.0072 |
| 547 | 0.0046 |
| 548 | 0.0064 |
| 549 | 0.0073 |
| 550 | 0.0055 |
| 551 | 0.0055 |
| 552 | 0.0053 |
| 553 | 0.0045 |
| 554 | 0.003 |
| 555 | 0.0047 |
| 556 | 0.0059 |
| 557 | >16.61 |
| 558 | 0.0041 |
| 559 | 0.0027 |
| 560 | 0.0034 |
| 561 | 0.0023 |
| 562 | 0.0027 |
| 563 | 0.0015 |
| 564 | 0.18 |
| 565 | >16.61 |
| 566 | >3.32 |
| 567 | 0.0031 |
| 568 | 0.39 |
| 569 | 0.0013 |
| 570 | 0.029 |
| 571 | 0.0035 |
| 572 | 0.0058 |
| 573 | 0.65 |
| 574 | 1.39 |

ND = no data

PBMC pSTAT3 assay

Cryopreserved peripheral blood mononuclear cells (PBMCs) from healthy donors were thawed and washed twice in ImmunoCult-XF T cell expansion medium (XF-TCEM) supplemented with CTL anti-aggregate wash. The cells were counted, resuspended at 2×105 cells per mL XF-TCEM supplemented with penicillin/streptomycin and 100 ng/mL IL-1 (BioLegend, 579404), and cultured in tissue culture flasks coated with anti-CD3 (eBioscience, 16-0037-85 or BD Pharmingen, 555329) at 37° C. in 5% CO2. On day 4 of culture, PBMCs were collected, washed twice in RPMI-1640 supplemented with 0.1% BSA (RPMI-BSA), and incubated in RPMI-BSA in upright tissue culture flasks for 4 hours at 37° C. in 5% CO2. Following this ‘starvation,’ a total of 6×104 cells in 30 pL RPMI-BSA was transferred into each well of a 384-well plate pre-spotted with peptide or DMSO. The cells were incubated for 30 minutes prior to the addition of IL-23 at a final concentration of 5 ng/mL. The cells were stimulated with cytokine for 30 minutes at 37° C. in 5% CO2, transferred onto ice for 10 minutes, and lysed. Cell lysates were stored at −80° C. until phosphorylated STAT3 was measured using the phospho-STAT panel kit (Meso Scale Discovery, K15202D). Results are provided below.

TABLE 4
PBMC pSTAT3 Data

| PBMC pSTAT3 IC50 (nM) | SEQ ID NO: |
|---|---|
| 7.6E−3 | 57 |
| 0.0081 | 62 |
| 0.012 | 68 |
| 0.0057 | 82 |
| 0.0032 | 104 |
| 0.0075 | 105 |
| 0.022 | 145 |
| 0.013 | 164 |
| 0.037 | 181 |
| 0.026 | 217 |
| 0.0075 | 281 |

### Pharmacokinetics Assay

### Exposed Polar Surface Area (EPSA)

### I. Protocol

Exposed Polar Surface Area (EPSA) is an experimental descriptor of molecular polarity obtained from chromatographic retention in supercritical fluid chromatography (SFC).

The stationary phase (Chirex 3014(S)-valine and (R)-1-(alpha-naphthyl) ethylamine) was selected for its balance of lipophilic and polar attributes and its capacity to separate compounds with wide polarity differences. The mobile phase consists of methanol (+Ammonium formate) and CO2. Polar compounds are retained stronger under these conditions and a low-slope gradient of methanol achieves elution based on increasing polarity of the mobile phase. Results are normalized using calibration standards generating a linear relationship between retention times and EPSA values. The unit of EPSA is Å².

Along with the measurement of compounds of interest, a series of 8 calibration standards (see Materials) are analyzed generating a linear relationship between retention times and EPSA value. The 8 compounds used as calibration standard have their own EPSA value defined by G. Goetz based on the comparison with TPSA (see G. Goetz, L. Philippe and M. Shapiro, “EPSA: A novel supercritical fluid chromatography technique enabling the design of permeable cyclic peptides,” ACS Med. Chem. Lett., vol. 5, pp. 1167-1172, 2014 for more information). The slope from the linear relationship between EPSA values of these eight compounds and their retention times is used to determine the EPSA value of each compound of interest that's measured under the same condition using the equation as below:

$$EPSA = {aT}_{R} + b \\ a : slope \\ b : intercept \\ {T}_{R} : retention time of the compound to be analyzed \qquad ( eq1 )$$

The mixture of calibrations standard is reinjected every 20 compounds to validate the stability of the retention time.

### II. Materials

### Chemicals

The mobile phase modifier consists of ammonium formate LC_MS grade (>99%) (CAS number 540-69-2) diluted to 20 mM in HPLC grade Methanol (MeOH, CAS number 67-56-1) and liquid C02 SFC grade (CAS number 124-38-9).

### Calibration Standards

Antipyrine (CAS number: 60-80-0) Chlorpromazine hydrochloride (CAS number: 69-09-0), desipramine hydrochloride (CAS number: 60-80-0), pindolol (CAS Number: 13523-86-9), diclofenac (CAS number: 15307-79-6)), m-nitro benzoic acid (CAS number: 121-92-6), bumetanide (CAS number: 28395-03-1), furosemide (CAS number: 54-31-9) were purchased from Sigma Aldrich (Steihneim, Germany).

### Instrumentation

All analyses were performed on a ultra performance convergence chromatography (UPC™) apparatus (Waters, Milford, MA, USA) equipped with a sample manager, a binary solvent delivery pump (BSM), a column manager, a convergence manager (automated back pressure regulator), a photo diode array detector (DAD) with a high pressure flow cell, and a mass detector simple quadrupole QDa. After the UV detector, the effluent of the SFC was mixed with an eluent consist of mixture MeOH/water (+0.3% Ammonia) 95/5 delivered by a make up pump with a flow rate of 0.4 mL/min and then split to the QDa using a splitter. The instrument was controlled, and data were processed with Masslynx 4.1.

### III. Methods and Procedures

Chromatographic conditions

The Analyses were performed using a 4.6×50 mm Chirex 3014(S)-valine and (R)-1-(alpha- naphthyl) ethylamine column (Phenomenex, Le Pecq, France) with 5 um particle and 100A pore size. The flow rate was 4 mL/min with the outlet pressure set to 14 MPa. The injection volume was 2 μL. the mobile phase composition was varied from 5% to 60% modifier at 20%/ min in a linear gradient, holding at 60% for 1.45 min and reverting to the original 5% until the end of the run. Data are acquired during 5 min. An equilibration time of 1 min is applied between 2 injections resulting in an injection every 6 min. each sample is analyzed in duplicate, resulting in a sample to sample turnaround time of 12 min.

The column temperature was set to 35° C. The mass detector was used in full scan from 100 to 800 for small molecules and from 500 to 1200 for peptides.

### Sample Preparation

Each sample is delivered by compound logistics from DMSO stock solution without dilution. The concentration is 1 mM and the volume is 60 μL. Results are provided below in Table 5.

TABLE 5
EPSA Data

| | EPSA | | EPSA |
|---|---|---|---|
| SEQ ID | Å² | SEQ ID | Å² |
| 1 | 179.2 | 18 | 218.64 |
| 2 | 184 | 19 | 170.68 |
| 3 | ND | 20 | 168.14 |
| 4 | ND | 21 | 170.27 |
| 5 | 168 | 22 | ND |
| 6 | 170 | 23 | ND |
| 7 | 170.25 | 24 | ND |
| 8 | 169.19 | 25 | ND |
| 9 | 186.57 | 26 | ND |
| 10 | ND | 27 | ND |
| 11 | ND | 28 | ND |
| 12 | ND | 29 | ND |
| 13 | 174.37 | 30 | 166.07 |
| 14 | 166.46 | 31 | 158.19 |
| 15 | 167.61 | 32 | 166.07 |
| 16 | 180.25 | 33 | ND |
| 17 | 186.49 | 34 | ND |
| 35 | 183.94 | 67 | 149.57 |
| 36 | ND | 68 | 148.49 |
| 37 | 174.37 | 69 | 161.12 |
| 38 | 171.73 | 70 | 156.65 |
| 39 | 157.5 | 71 | 146.31 |
| 40 | 156.45 | 72 | 159.64 |
| 41 | ND | 73 | 155.28 |
| 42 | ND | 74 | 154.47 |
| 43 | 172.79 | 75 | 157.19 |
| 44 | ND | 76 | 161.27 |
| 45 | ND | 77 | 161.27 |
| 46 | 161.58 | 78 | 157.19 |
| 47 | 154.45 | 79 | 144.02 |
| 48 | 160.24 | 80 | 157.19 |
| 49 | 164.21 | 81 | 173.1 |
| 50 | 162.27 | 82 | 170.9 |
| 51 | 155.73 | 83 | 167.08 |
| 52 | 158.35 | 84 | 165.97 |
| 53 | 154.86 | 85 | 150.94 |
| 54 | 155.73 | 86 | 150.38 |
| 55 | 158.79 | 87 | 159.29 |
| 56 | 157.91 | 88 | 157.62 |
| 57 | 165.68 | 89 | 159.84 |
| 58 | 153.55 | 90 | 164.41 |
| 59 | 166.13 | 91 | 145.32 |
| 60 | 157.48 | 92 | 151.44 |
| 61 | 159.22 | 93 | 150.66 |
| 62 | 159.66 | 94 | 141.79 |
| 63 | 156.6 | 95 | 131.48 |
| 64 | 159.66 | 96 | 138.7 |
| 65 | 153.99 | 97 | 152.32 |
| 66 | 138.15 | 98 | 139.17 |
| 99 | 164.31 | 131 | 181.72 |
| 100 | 139.21 | 132 | 162.77 |
| 101 | 144.77 | 133 | 151.2 |
| 102 | 155.87 | 134 | 158.67 |
| 103 | 157.54 | 135 | 171.46 |
| 104 | 149.76 | 136 | 169.08 |
| 105 | 155.32 | 137 | 159.9 |
| 106 | 153.1 | 138 | 158.69 |
| 107 | 172.53 | 139 | 150.04 |
| 108 | 173.08 | 140 | 148.3 |
| 109 | 171.42 | 141 | 139.7 |
| 110 | 170.86 | 142 | 166.39 |
| 111 | 152.54 | 143 | 160.85 |
| 112 | 169.2 | 144 | 160.85 |
| 113 | 168.93 | 145 | 157.79 |
| 114 | 172.88 | 146 | 151.73 |
| 115 | 171.09 | 147 | 155.9 |
| 116 | 169.51 | 148 | 153.15 |
| 117 | 139.35 | 149 | 157.43 |
| 118 | 137.77 | 150 | 158.85 |
| 119 | 159.3 | 151 | 182.16 |
| 120 | 153.09 | 152 | 155.29 |
| 121 | 145.46 | 153 | 170.71 |
| 122 | 140.83 | 154 | 143.9 |
| 123 | 156.09 | 155 | 150.02 |
| 124 | 158.35 | 156 | 154.54 |
| 125 | 181.3 | 157 | 149.27 |
| 126 | 183.67 | 158 | 160.34 |
| 127 | 178.77 | 159 | 149.43 |
| 128 | 174.51 | 160 | 151.21 |
| 129 | 165.09 | 161 | 175.05 |
| 130 | 164.24 | 162 | 162.6 |
| 163 | 167.7 | 195 | 146.63 |
| 164 | 144.54 | 196 | 149.05 |
| 165 | 151.37 | 197 | 136.79 |
| 166 | 178.67 | 198 | 144 |
| 167 | 157.24 | 199 | 140.82 |
| 168 | 154.26 | 200 | 144.93 |
| 169 | 153.78 | 201 | 145.2 |
| 170 | 145.98 | 202 | 145.69 |
| 171 | 152.42 | 203 | 135.46 |
| 172 | 144.57 | 204 | 152.51 |
| 173 | 144.3 | 205 | 148.54 |
| 174 | 158.02 | 206 | 144.51 |
| 175 | 140.17 | 207 | 139.38 |
| 176 | 168.08 | 208 | 141.12 |
| 177 | 148.55 | 209 | 138.98 |
| 178 | 154.36 | 210 | 144.81 |
| 179 | 147.3 | 21 | 151.8 |
| 180 | 150.1 | 212 | 138.51 |
| 181 | 136.18 | 213 | 154.42 |
| 182 | 152.68 | 214 | 151.32 |
| 183 | 132.92 | 215 | 144.6 |
| 184 | 141.61 | 216 | 141.4 |
| 185 | 155.44 | 217 | 138.09 |
| 186 | 147.88 | 218 | 136.46 |
| 187 | 137.31 | 219 | 139.14 |
| 188 | 146.8 | 220 | 142.4 |
| 189 | 144.34 | 221 | 134.2 |
| 190 | 136.64 | 222 | 142.87 |
| 191 | 143.97 | 223 | 140.66 |
| 192 | 148.89 | 224 | 138.88 |
| 193 | 134.97 | 225 | 136.15 |
| 194 | 146.09 | 226 | 141.61 |
| 227 | 139.24 | 259 | 148.99 |
| 228 | 139.19 | 260 | 147.41 |
| 229 | 145.23 | 26 | 147.98 |
| 230 | 141.56 | 262 | 144.75 |
| 231 | 146.63 | 263 | 139.95 |
| 232 | 140.07 | 264 | 150.97 |
| 233 | 140.57 | 265 | 158.91 |
| 234 | 140.51 | 266 | 157.49 |
| 235 | 149.11 | 267 | ND |
| 236 | 158.53 | 268 | 147.97 |
| 237 | 140.29 | 269 | 167.1 |
| 238 | 145.03 | 270 | ND |
| 239 | 138.47 | 271 | 159.03 |
| 240 | 143.71 | 272 | 146.28 |
| 241 | 134.84 | 273 | 157.24 |
| 242 | 144.75 | 274 | 153.32 |
| 243 | 141.56 | 275 | 149.4 |
| 244 | 137.65 | 276 | ND |
| 245 | 148.01 | 277 | 153.84 |
| 246 | 150.87 | 278 | 177.76 |
| 247 | 144.7 | 279 | 163.04 |
| 248 | 145.58 | 280 | 167.45 |
| 249 | 147.12 | 281 | 157.66 |
| 250 | 145.58 | 282 | 163.09 |
| 251 | 147.79 | 283 | 146.05 |
| 252 | 147.67 | 284 | 150.78 |
| 253 | 145.75 | 285 | 147.73 |
| 254 | 143.54 | 286 | 148.94 |
| 255 | 140.84 | 287 | ND |
| 256 | 141.94 | 288 | 144.38 |
| 257 | 157.08 | 289 | 142.36 |
| 258 | 151.09 | 290 | 137.35 |
| 291 | 136.71 | 323 | ND |
| 292 | 151.88 | 324 | ND |
| 293 | 149.22 | 325 | ND |
| 294 | 157.01 | 326 | 156.77 |
| 295 | 149.85 | 327 | 156.65 |
| 296 | ND | 328 | 159.08 |
| 297 | 152.93 | 329 | 155.2 |
| 298 | 149.43 | 330 | 153.68 |
| 299 | 157.14 | 331 | 152.9 |
| 300 | 153.44 | 332 | 157.74 |
| 301 | 153.2 | 333 | 158.53 |
| 302 | 155.02 | 334 | 154.84 |
| 303 | 152.11 | 335 | 152.77 |
| 304 | 164.23 | 336 | 155.68 |
| 305 | 152.65 | 337 | 151.68 |
| 306 | 152.77 | 338 | ND |
| 307 | ND | 339 | 154.59 |
| 308 | 154.17 | 340 | 155.02 |
| 309 | 157.5 | 341 | 152.77 |
| 310 | 152.35 | 342 | 161.75 |
| 311 | 156.23 | 343 | 156.47 |
| 312 | 151.08 | 344 | 157.74 |
| 313 | ND | 345 | 161.93 |
| 314 | ND | 346 | 157.68 |
| 315 | ND | 347 | 186.53 |
| 316 | 153.62 | 348 | 184.27 |
| 317 | 157.99 | 349 | 178.87 |
| 318 | 150.83 | 350 | ND |
| 319 | 150.96 | 351 | 184.06 |
| 320 | 161.57 | 352 | 178.87 |
| 321 | 152.83 | 353 | ND |
| 322 | ND | 354 | 194.35 |
| 355 | 186 | 387 | ND |
| 356 | 173.56 | 388 | 161.08 |
| 357 | 180.66 | 389 | 185.39 |
| 358 | 141.77 | 390 | 147.59 |
| 359 | 146.57 | 391 | 158.02 |
| 360 | 166.39 | 392 | ND |
| 361 | 168.49 | 393 | 144.09 |
| 362 | 156.88 | 394 | ND |
| 363 | 155.02 | 395 | 189.15 |
| 364 | 156.34 | 396 | 171.59 |
| 365 | 150.35 | 397 | ND |
| 366 | 161.66 | 398 | 167.78 |
| 367 | 161.72 | 399 | 157.12 |
| 368 | 166.1 | 400 | 172.62 |
| 369 | 183.72 | 401 | 155.02 |
| 370 | 186.97 | 402 | 156.32 |
| 371 | 181.81 | 403 | ND |
| 372 | 203.32 | 404 | 195.62 |
| 373 | ND | 405 | 157.04 |
| 374 | 170.21 | 406 | 149.57 |
| 375 | 171.72 | 407 | 151.45 |
| 376 | 176.07 | 408 | 165.31 |
| 377 | 148.25 | 409 | 197.81 |
| 378 | 149.15 | 410 | ND |
| 379 | 149.74 | 411 | 155.37 |
| 380 | 170.86 | 412 | 156.86 |
| 381 | 171.07 | 413 | 153.88 |
| 382 | 176.03 | 414 | 156.56 |
| 383 | ND | 415 | 147.97 |
| 384 | 156.76 | 416 | 166.44 |
| 385 | ND | 417 | 166.56 |
| 386 | ND | 418 | 166.5 |
| 419 | 155.68 | 451 | 171.93 |
| 420 | 170.15 | 452 | 163.23 |
| 421 | 167.63 | 453 | 173.31 |
| 422 | 192.5 | 454 | 175.89 |
| 423 | 160.53 | 455 | 158.19 |
| 424 | 164.85 | 456 | 156.45 |
| 425 | 160.71 | 457 | 163.17 |
| 426 | 163.05 | 458 | 171.51 |
| 427 | 167.73 | 459 | 178.23 |
| 428 | 160.59 | 460 | 166.65 |
| 429 | 191.54 | 461 | 192.25 |
| 430 | 166.83 | 462 | ND |
| 431 | 174.03 | 463 | 150.69 |
| 432 | 167.19 | 464 | 160.46 |
| 433 | 163.53 | 465 | 175.8 |
| 434 | 155.97 | 466 | 153.42 |
| 435 | 181.7 | 467 | 153.6 |
| 436 | 149.44 | 468 | 151.42 |
| 437 | 185.54 | 469 | 156.04 |
| 438 | 167.49 | 470 | 159.61 |
| 439 | 166.59 | 471 | 174.1 |
| 440 | 162.69 | 472 | ND |
| 441 | 175.65 | 473 | 201.93 |
| 442 | 169.17 | 474 | 185.44 |
| 443 | 191.48 | 475 | 153.1 |
| 444 | 174.09 | 476 | 154.87 |
| 445 | 175.65 | 477 | 165.99 |
| 446 | 172.65 | 478 | 163.79 |
| 447 | 170.73 | 479 | 157.51 |
| 448 | 175.53 | 480 | 197.79 |
| 449 | 175.71 | 48 | 183.86 |
| 450 | 164.85 | 482 | 180.35 |
| 483 | 165.46 | 515 | 197.29 |
| 484 | 163.28 | 516 | 236.66 |
| 485 | 163.28 | 517 | 240.66 |
| 486 | 164.13 | 518 | 229.77 |
| 487 | 164.43 | 519 | 186.31 |
| 488 | 164.55 | 520 | ND |
| 489 | 163.71 | 521 | ND |
| 490 | 182.11 | 522 | 175.57 |
| 491 | 168.13 | 523 | 154.05 |
| 492 | 164.55 | 524 | 198.42 |
| 493 | 132.09 | 525 | 195 |
| 494 | 132.79 | 526 | 184.45 |
| 495 | 160.2 | 527 | 188.74 |
| 496 | 161.47 | 528 | 162.7 |
| 497 | 161.71 | 529 | 161 |
| 498 | 161.66 | 530 | 166.18 |
| 499 | 133.49 | 531 | 165.96 |
| 500 | 159.17 | 532 | 171.46 |
| 501 | 174.72 | 533 | 170.43 |
| 502 | 168.79 | 534 | 171.76 |
| 503 | 169.34 | 535 | 189.92 |
| 504 | 178.11 | 536 | 210.36 |
| 505 | 178.96 | 537 | 163.06 |
| 506 | 190.82 | 538 | 163.06 |
| 507 | 163.59 | 539 | ND |
| 508 | 197.17 | 540 | 182.65 |
| 509 | 160.98 | 541 | 186.77 |
| 510 | 201.18 | 542 | 179.56 |
| 511 | 197.55 | 543 | 178.52 |
| 512 | 204.76 | 544 | 157.08 |
| 513 | 223.62 | 545 | 153.73 |
| 514 | 248.45 | 546 | 167.79 |
| 547 | 167.55 | | |
| 548 | ND | | |
| 553 | 184.93 | | |
| 554 | 171.4 | | |
| 555 | 170.67 | | |
| 556 | 162.87 | | |
| 557 | 138.57 | | |
| 558 | 173.74 | | |
| 559 | 154.7 | | |
| 560 | 153.66 | | |
| 561 | 162.68 | | |
| 562 | 165.55 | | |
| 563 | 193.71 | | |
| 564 | 152.69 | | |
| 565 | 143.6 | | |
| 566 | 136.63 | | |
| 567 | 171.63 | | |
| 568 | 173.73 | | |
| 569 | 172.95 | | |
| 570 | 154.38 | | |
| 571 | ND | | |
| 572 | 165.67 | | |
| 573 | 153.15 | | |
| 574 | ND | | |

ND: Not determined

Although the foregoing invention has been described in some detail by way of illustration and Example for purposes of clarity of understanding, one of skill in the art will appreciate that certain changes and modifications may be practiced upon review of the specification and within the scope of the appended claims. The full scope of the invention should be determined by reference to the claims, along with their full scope of equivalents, and the specification, along with such variations. In addition, each reference provided herein is incorporated by reference in its entirety to the same extent as if each reference was individually incorporated by reference. Where a conflict exists between the instant application and a reference provided herein, the instant application shall dominate.

### Aspects of the Present Invention

The following aspects illustrate and are not intended to limit scope of the present invention. Instead, these aspects provide guidance to any skilled artisan on how to prepare and use compounds, compositions and methods taught by the present invention, where such skilled artisans will appreciate that modifications may be made without departing from the spirit and scope of the invention.

In one aspect of the invention:

1. A compound of Formula (I):

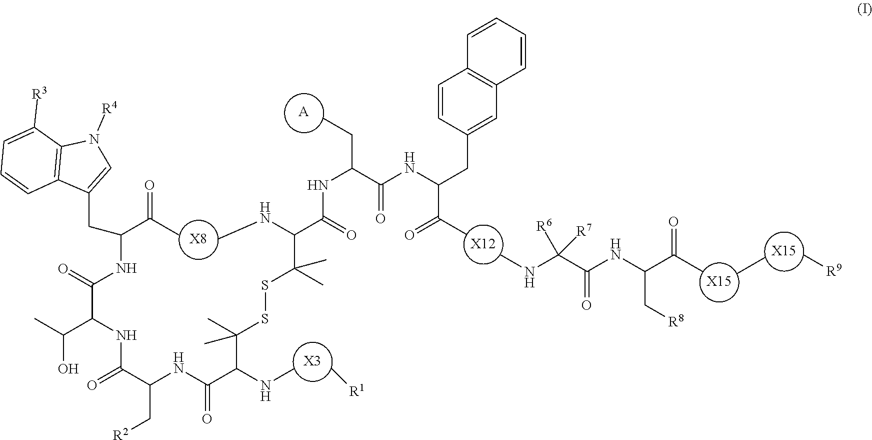

wherein:

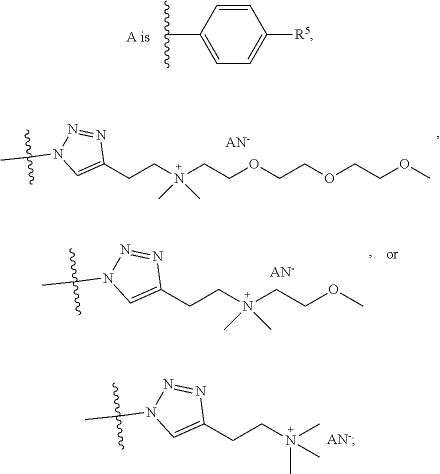

wherein:
R⁵ is —OCHF₂, —O(CH₂)₉CO₂H,

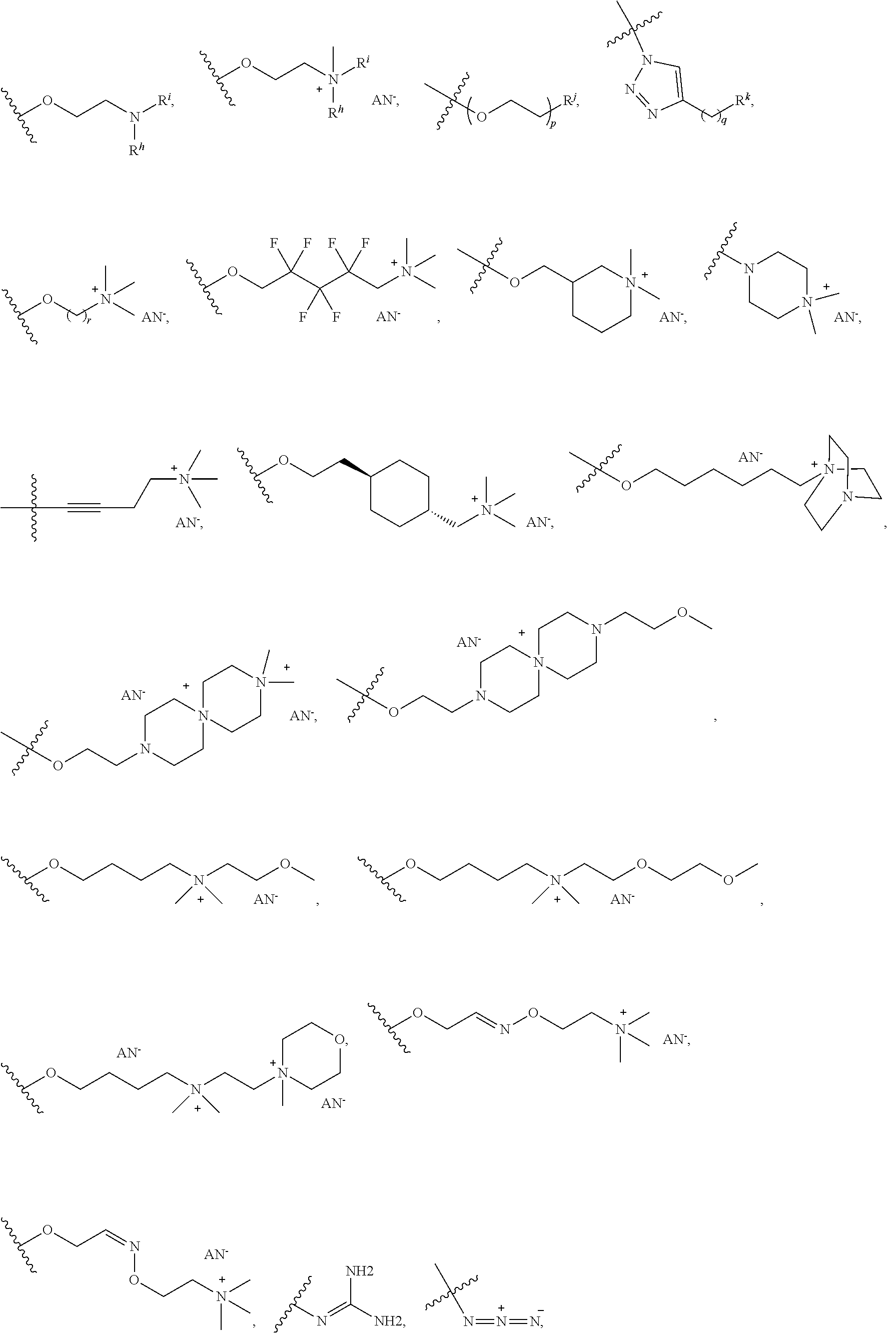

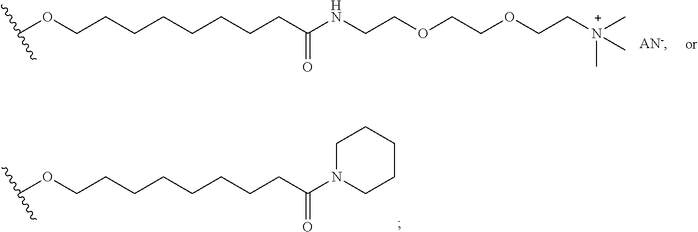

wherein:
R^{h} is H, —CH₃, —C(O)CH₃, —C(NH)NH₂, —(CH₂)₃O(CH₂)₂OCH₃, —CH₂CH₂OCH₃, —(CH₂CH₂O)₃CH₃, —(CH₂CH₂O)₆CH₃,

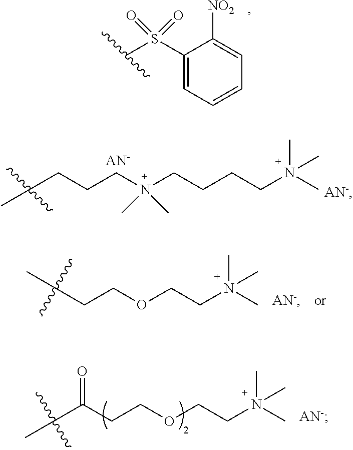

Rⁱ is H, —CH₃, —(CH₂)₃O(CH₂)₂OCH₃,

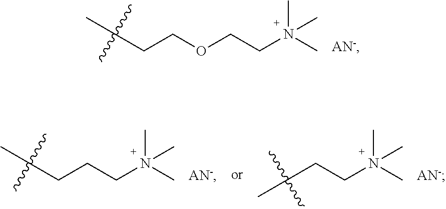

p is 1, 2 or 3;

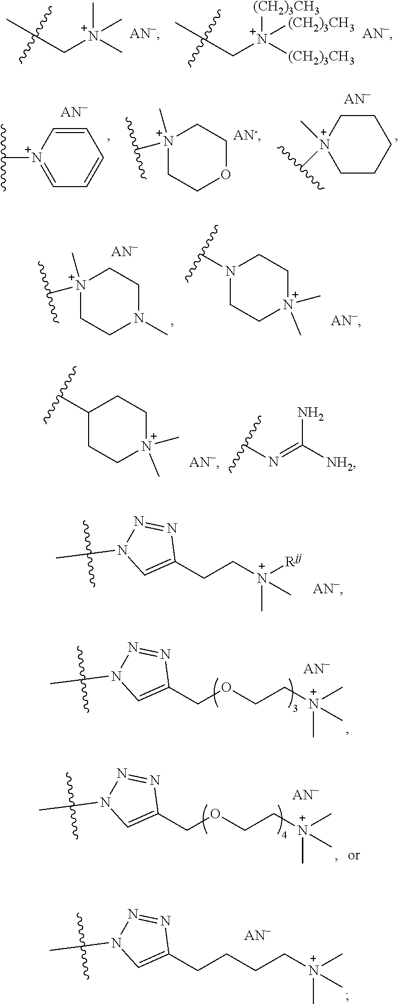

q is 1, 2, 3, 4, or 5;
R^{k} is —OCH₃,

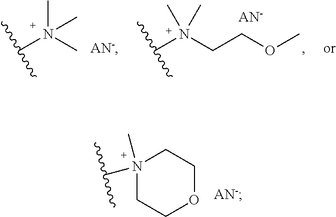

r is 3, 4, 5, 6, or 8;
wherein:
   R^{jj} is H, —(CH₂)₇CH₃, —(CH₂)₁₅CH₃, —(CH₂)₂OCH₃, or —(CH₂CH₂O)₃CH₃;
R¹ is —C(O)C(1-2)alkyl, —C(O)CF₃, —COCH₂CF₃, —CO(CH₂)₅N(CH₃)₃⁺AN⁻, —CH₂CH₂NHC(O)CH₂O(CH₂CH₂O)₂CH₃, —CH₂CH₂NHC(O)(CH₂CH₂O)₆CH₃,

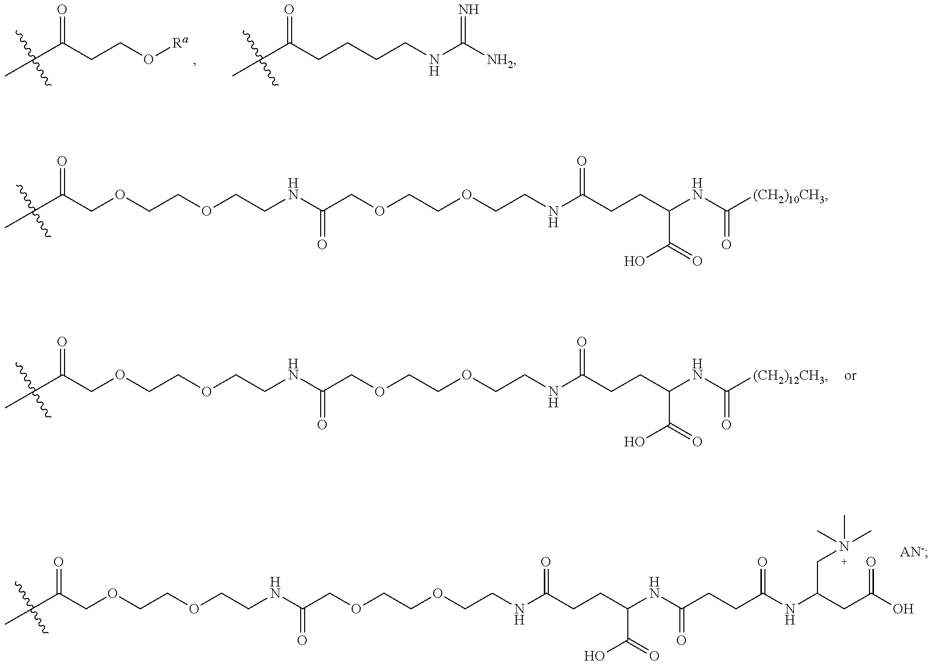

wherein:
R^{a} is

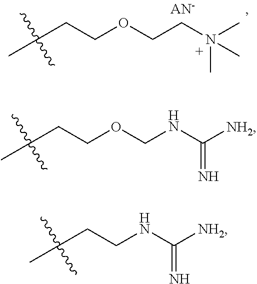

CH₂CH₂OCH₂CH₂N(CH₃)₂,
—(CH₂CH₂O)₃CH₂CH₂N(CH₃)₂, —(CH₂CH₂O)₃CH₂CH₂N(CH₃)₃ ⁺AN⁻, —(CH₂CH₂O)₅CH₂CH₂NC(O)CH₃, —(CH₂CH₂O)₈CH₂CH₂NC(O)CH₃, or —(CH₂CH₂O)₁₁CH₂CH₂NC(O)CH₃;
X3 is a direct bond, (D) arginine, (D) lysine substituted with R^{b},

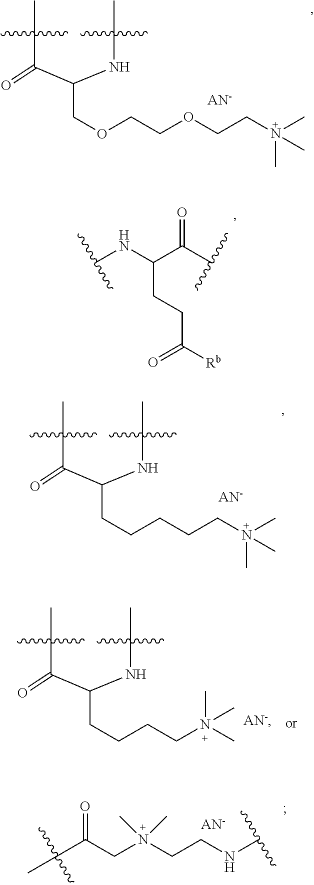

wherein:

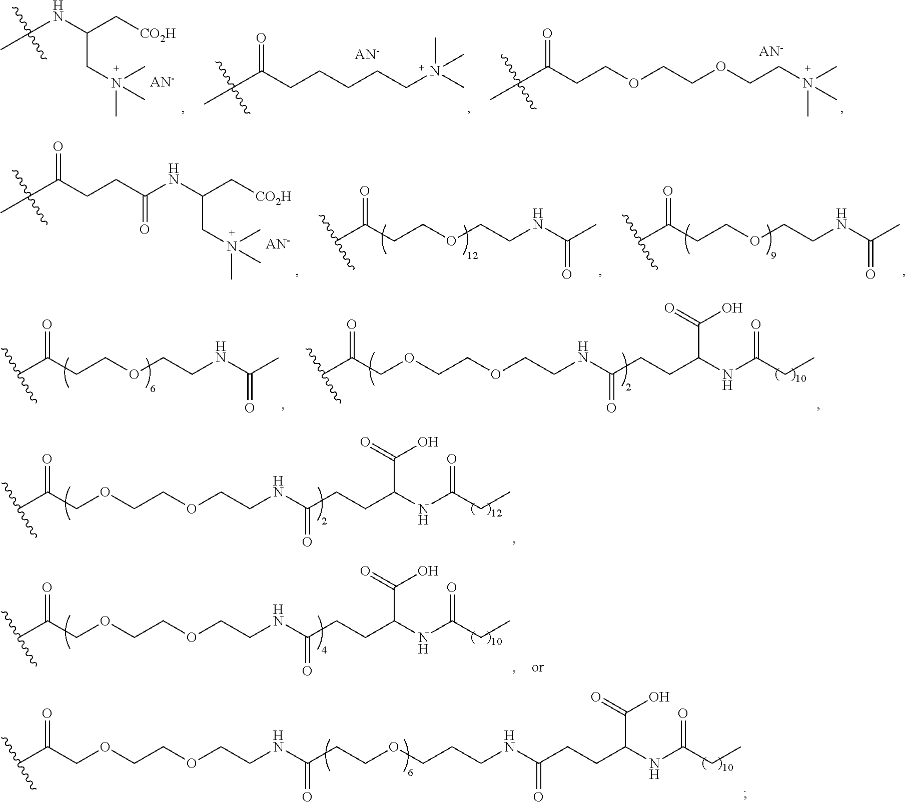

R² is H, —CH(CH₃)₂,

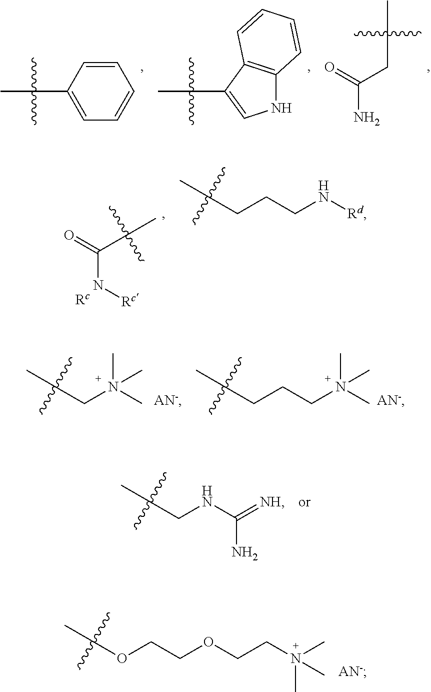

wherein:
R^{c} is H, or —CH₃
R^{c}′ is H, or —CH₃

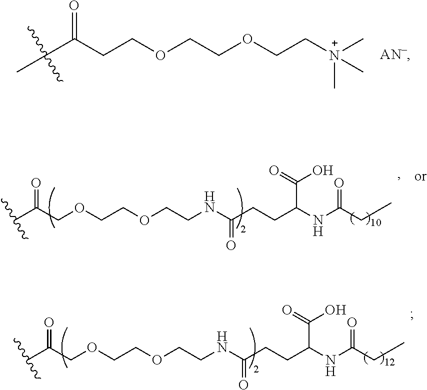

R³ is H, —CH₃, —CF₃, or

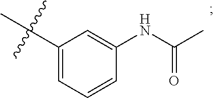 [SMILES: *c1cccc(NC(C)=O)c1]

R⁴ is H, or —CH₃;
X8 is

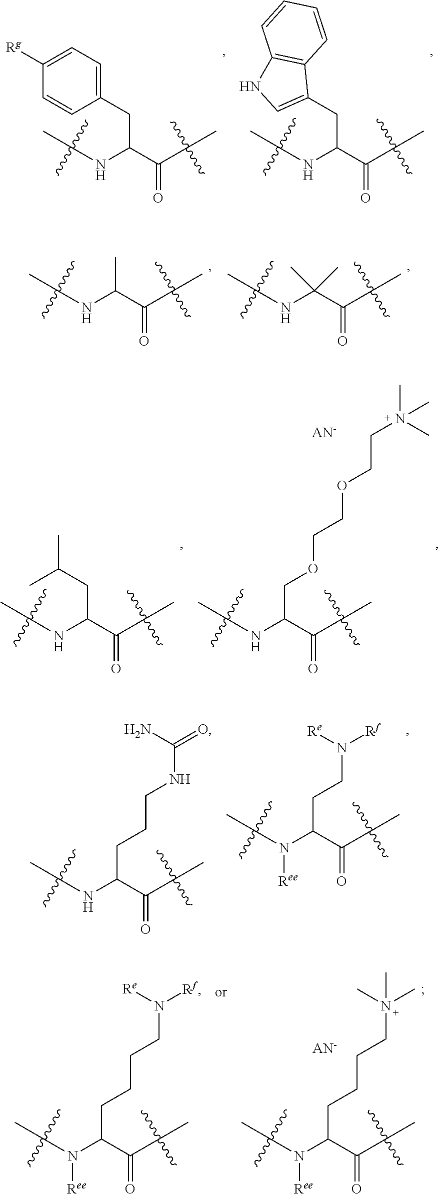

wherein:
   R^{e} is H, or —CH₃;
   R^{ee} is H;
   R^{f} is H, —CH₃, —C(CH₃)₃, —C(O)CH₃, —C(O)(CH₂CH₂O)₆CH₃, —(CH₂CH₂O)₆CH₃,

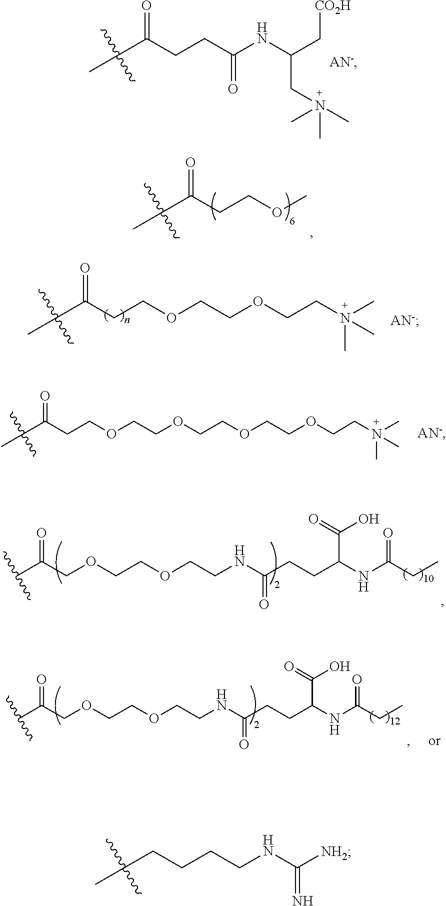

R^{g} is H, —OH, —C(O)NH₂, or —CH₂NHC(O)CH₃;
n is, or 1
X12 is absent,

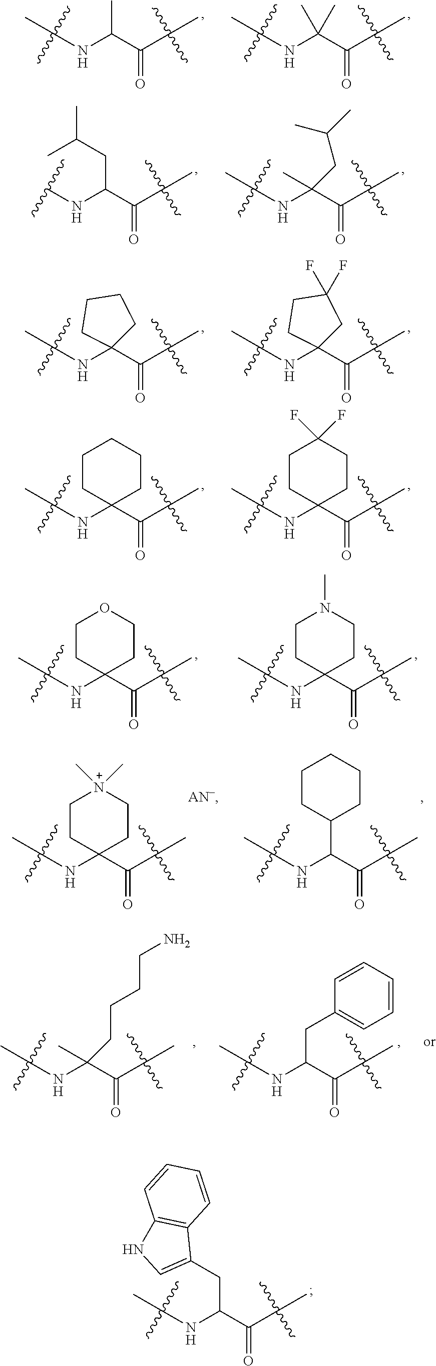

R⁶ is —CH₃, —CH₂CH₂C(O)R^{m}, —(CH₂)ₛNRⁿR^{o},

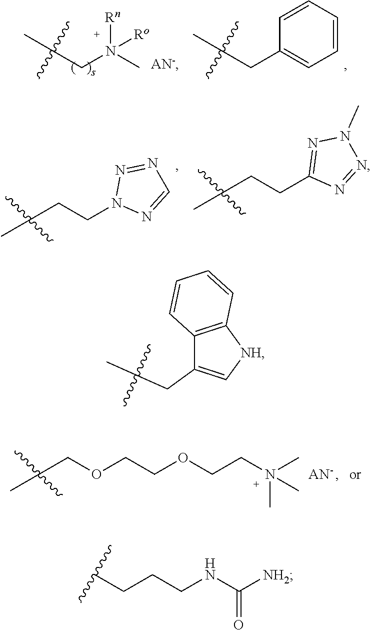

R⁷ is H, or —CH₃;
   wherein:
R^{m} is —OH, or

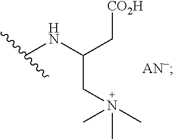

Rⁿ is H, —CH₃, —CH₂CH(CH₃)₂, —C(O)CH₃, —C(O)(CH₂CH₂O)₆CH₃, —(CH₂CH₂O)₆CH₃, —C(O)(CH₂CH₂O)ₜCH₂CH₂R^{p},

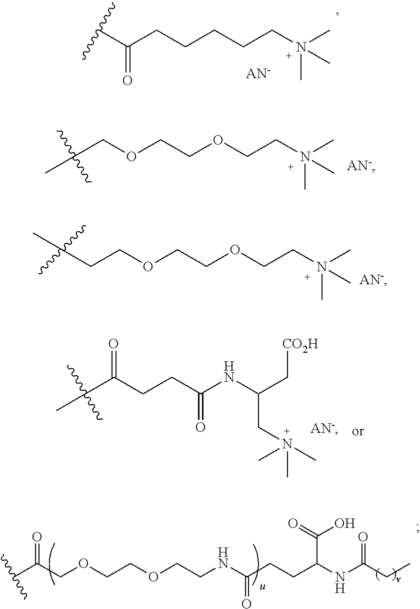

R^{o} is H, —CH₃, or —C(O)CH₃;
   wherein:
      R^{p} is NHC(O)CH₃, or

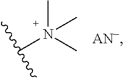

s is 1, 2, or 4;
t is 2, 6, 9, or 12;
u is 2 or 3;
v is 10 or 12;
R⁸ is —CH₂CH₂CH₂NHC(O)CH₃, —C(O)NH₂, or —C(O)NHCH₃;
X15 is absent,

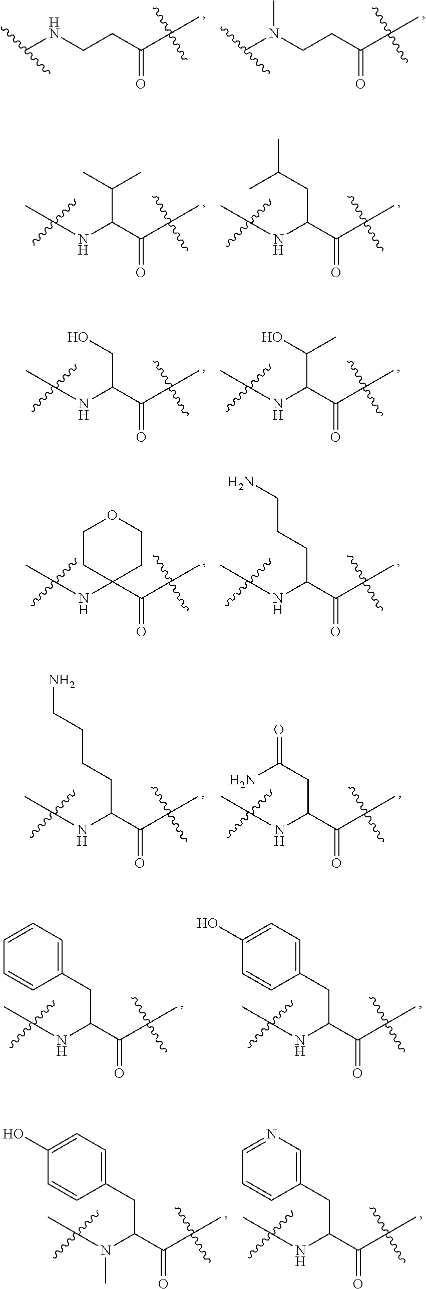

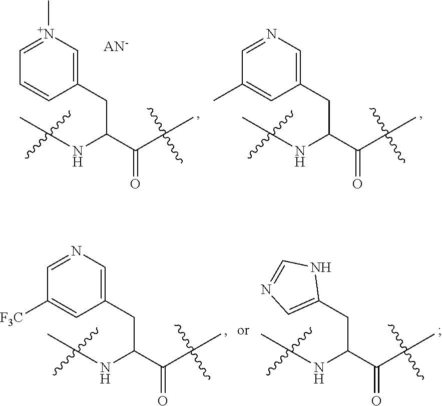 [SMILES: *NC(Cc1ccc[n+](C)c1)C(*)=O ; *NC(Cc1cncc(C)c1)C(*)=O ; *NC(Cc1cncc(C(F)(F)F)c1)C(*)=O ; *NC(Cc1cnc[nH]1)C(*)=O]

X16 is absent,

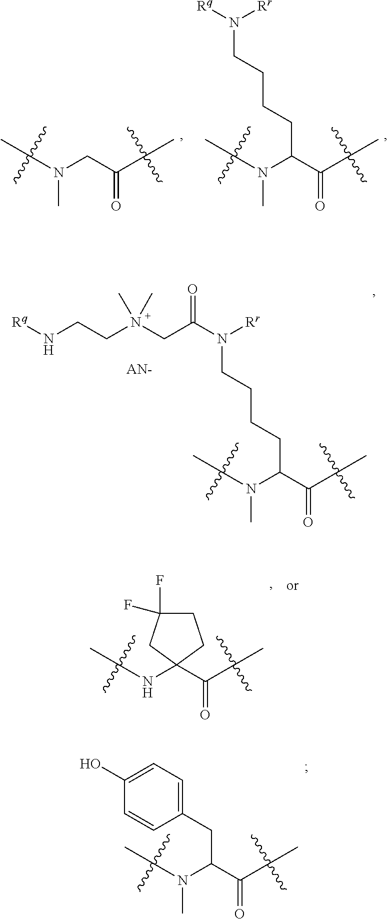

wherein:
   R^{q} is 0

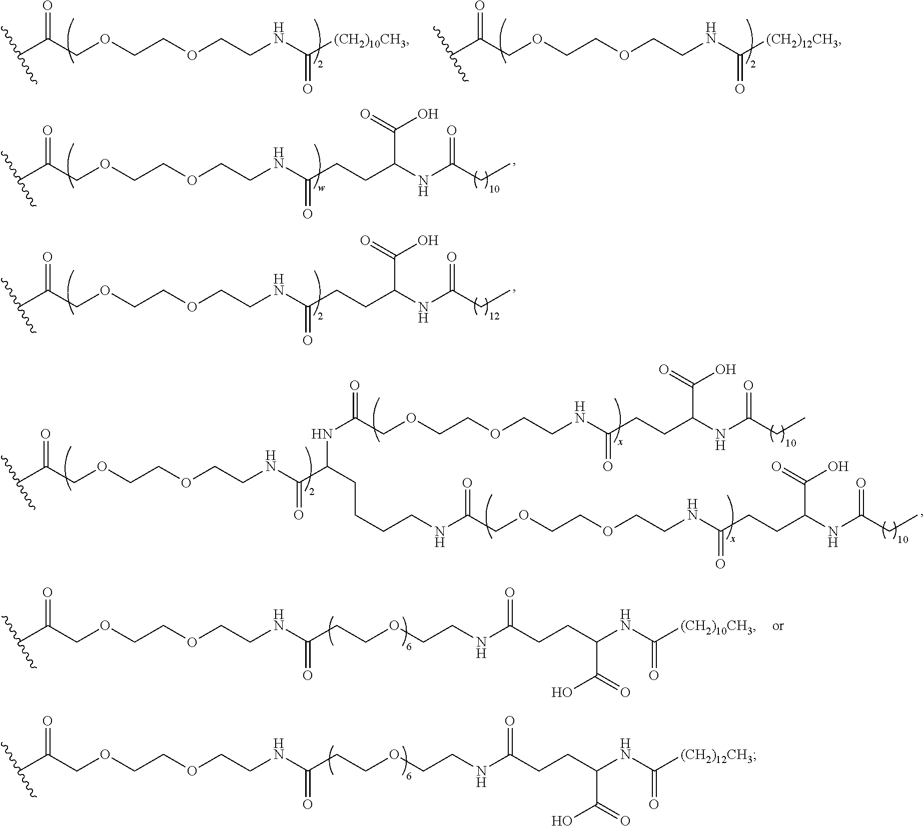

R^{r} is H;
w is24, or 5;
x is 2 or 4; and
   R⁹ is —NH₂, —NHCH₃, —N(CH₃)₂, —NH(CH₂CH₂O)₂CH₃, —N(CH₃)(CH₂CH₂O)₂CH₃, —NH(CH₂CH₂O)_{y}R^{s},

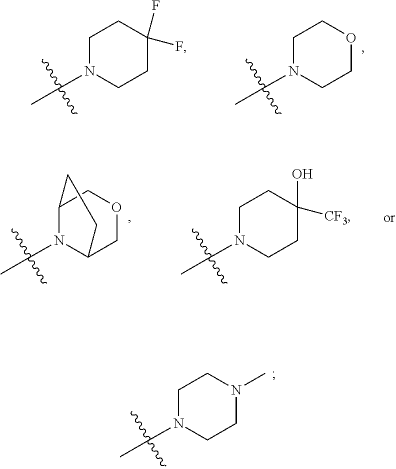

wherein:
   y is 2, 4, or 6;
   R^{s} is —CH₃, or

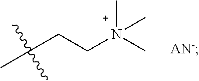 [SMILES: *CC[N+](C)(C)C]

wherein:
AN⁻as above defined for A, R, X3, X12, R1, R2, R5, R6, R8, R15, R16 and R^{a}, R^{b}, R^{d}, R^{f}, R^{h}, Rⁱ, R^{j}, R^{k}, R^{m}, Rⁿ, R^{o}, R^{s} is acetate, adipate, benzoate, benzenesulfonate, citrate, decanoate, chloride, lactate, maleate, methanesulfonate, oxalate, pivalate, propionate, succinate, sulfate, tartrate, or trifluoroacetate; or a pharmaceutically acceptable salt thereof.

In another aspect of the invention:

2. A compound of Formula (II)

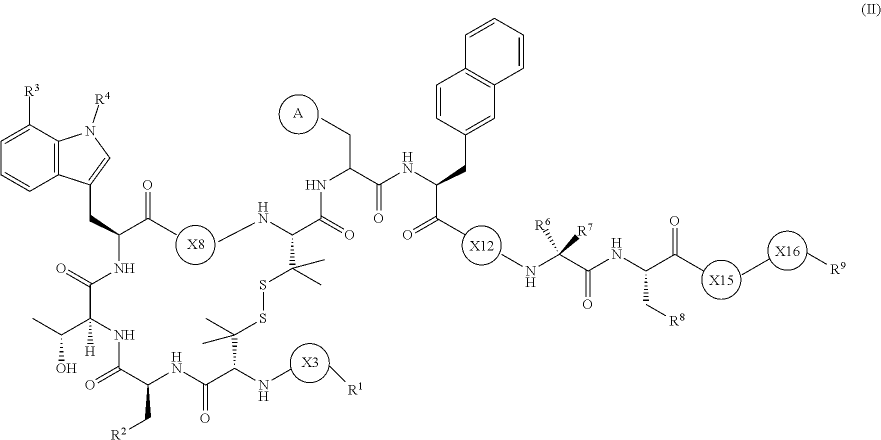

wherein
X12 is absent,

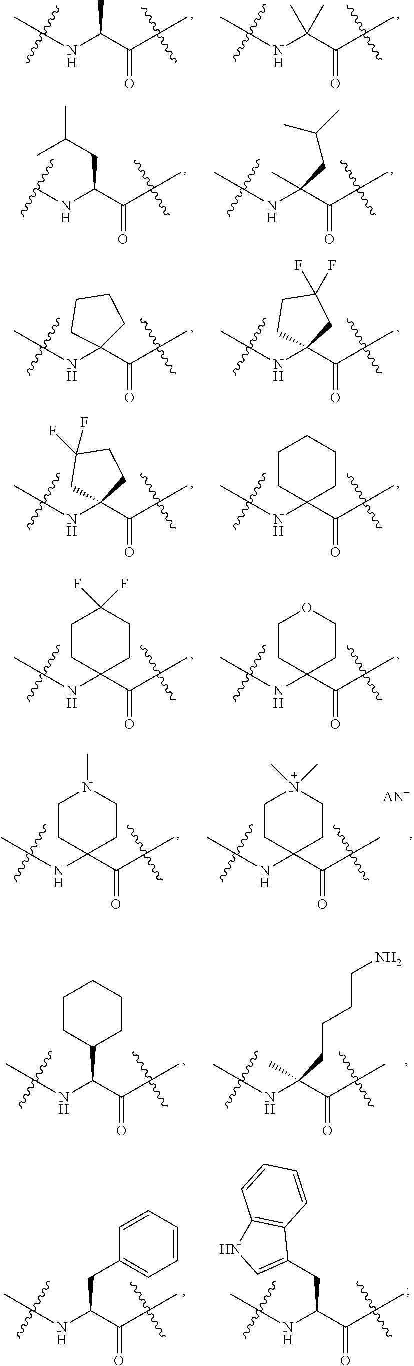

or a pharmaceutically acceptable salt thereof.

In another aspect of the invention:

3. The compound of aspect 2, wherein:
wherein:
A is

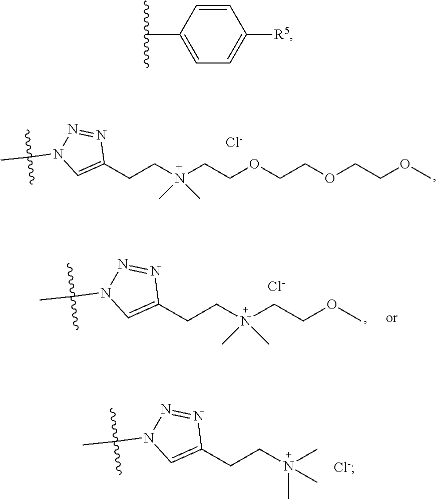

wherein:
   R⁵ is —OCHF₂, —O(CH₂)₉CO₂H,

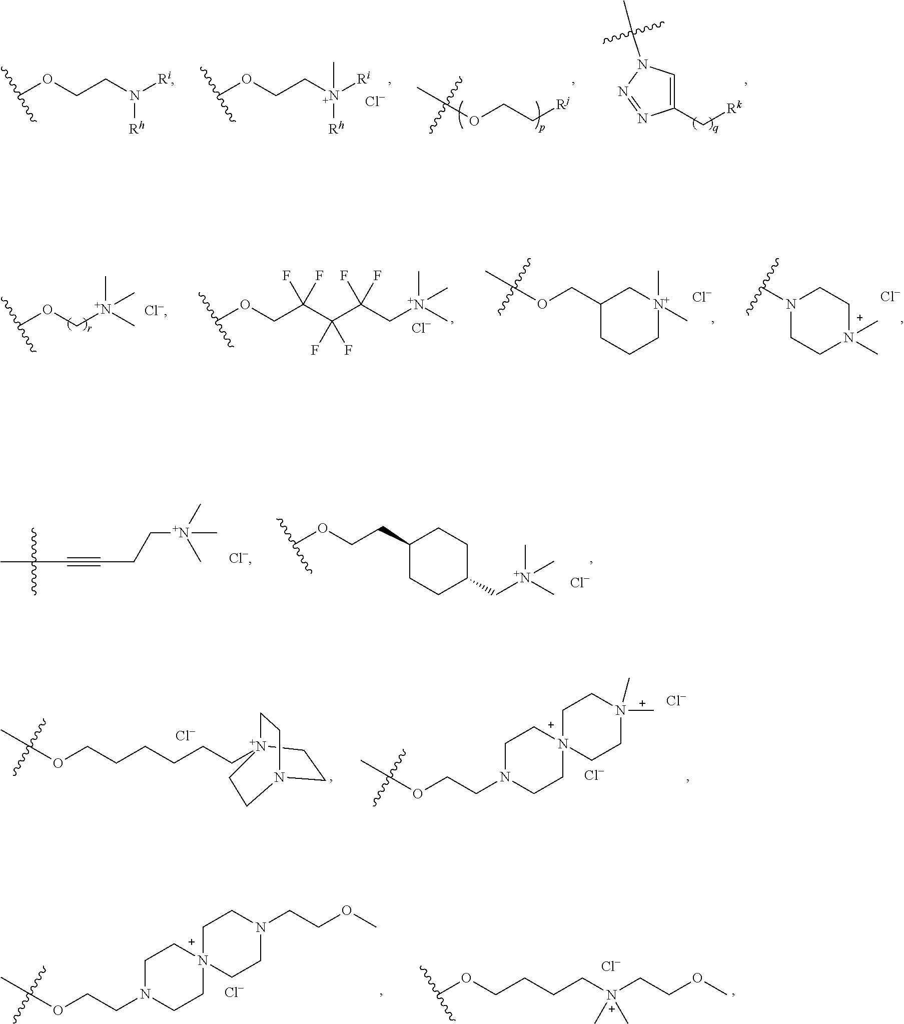

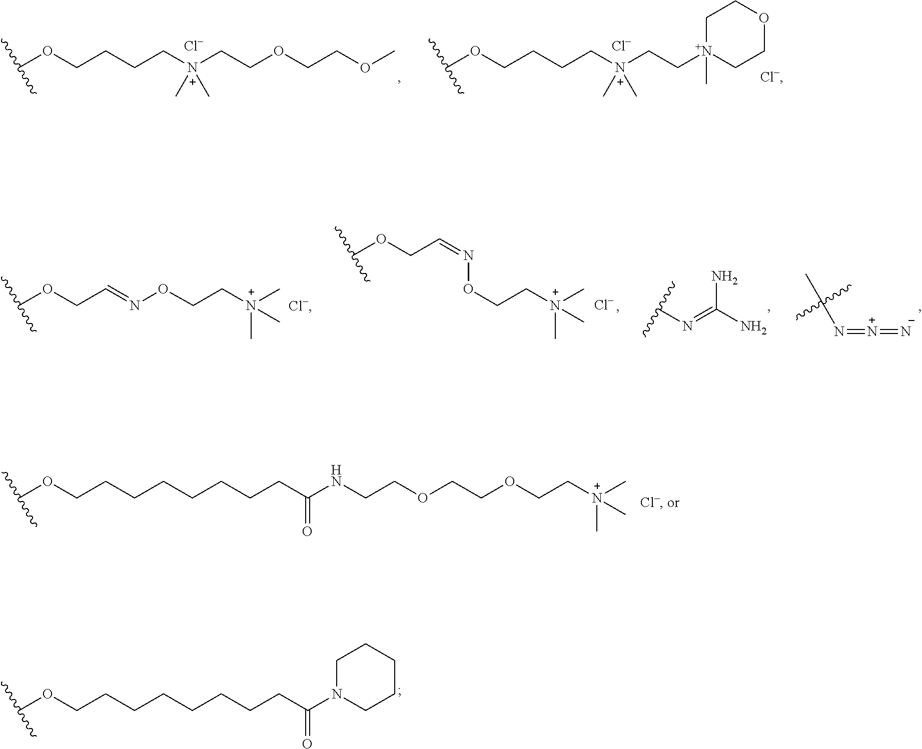

wherein:
   R^{h} is H, —CH₃, —C(O)CH₃, —C(NH)NH₂, —(CH₂)₃₀(CH₂)₂₀CH₃, —CH₂CH₂OCH₃, —(CH₂CH₂O)₃CH₃, —(CH₂CH₂O)₆CH₃,

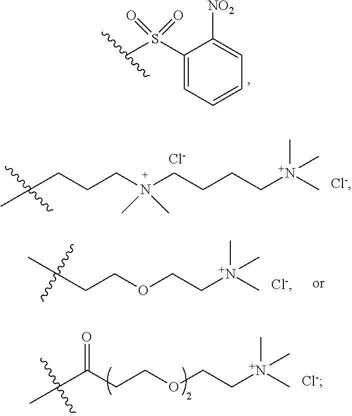

Rⁱ is H —CH₃, —(CH₂)₃O(CH₂)₂OCH₃,

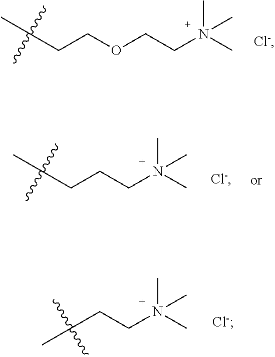

p is 1, 2 or 3;
R^{j} is

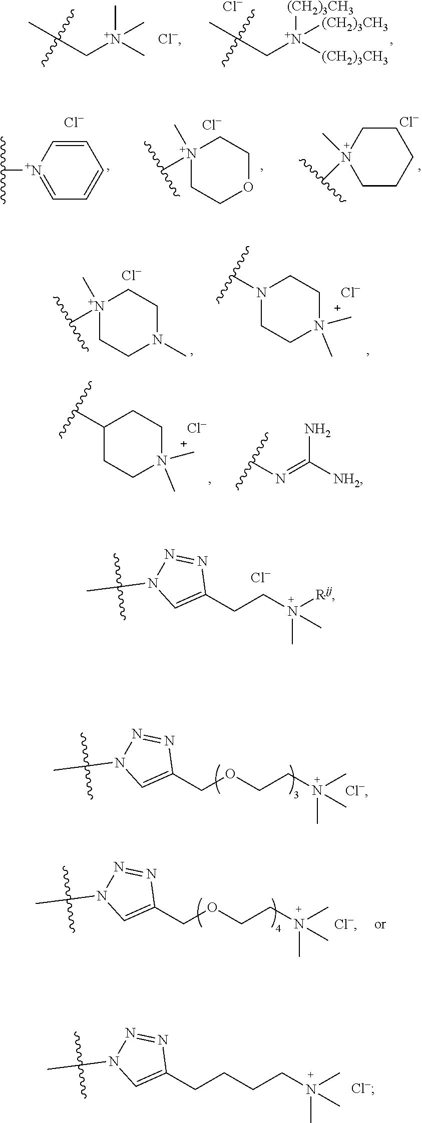

q is 1, 2, 3, 4, or 5;
R^{k} is —OCH₃,

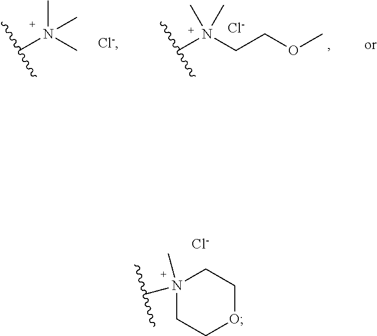

r is 3, 4, 5, 6, or 8;
wherein:
   R^{jj} is H, —(CH₂)₇CH₃, —(CH₂)₁₅CH₃, —(CH₂)₂OCH₃, or —(CH₂CH₂O)₃CH₃; R¹ is —C(O)C(1-2)alkyl, —C(O)CF₃, —COCH₂CF₃, —CO(CH₂)₅N(CH₃)₃⁺Cl⁻, —CH₂CH₂NHC(O)CH₂O(CH₂CH₂O)₂CH₃, —CH₂CH₂NHC(O)(CH₂CH₂O)₆CH₃,

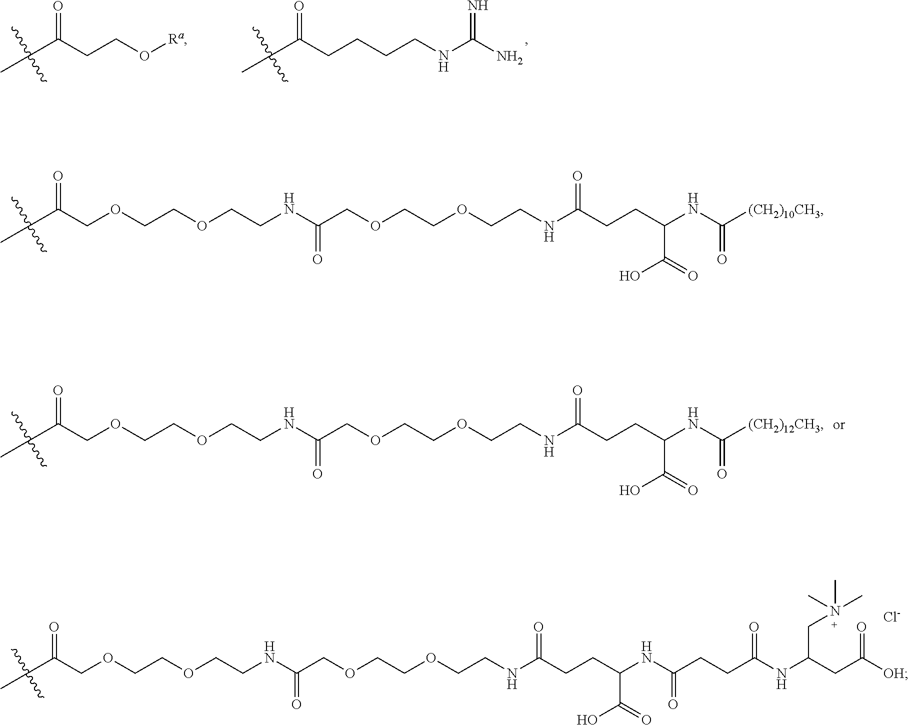

wherein:
   R^{a} is

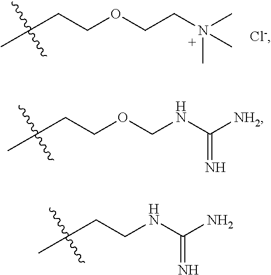

CH₂CH₂OCH₂CH₂N(CH₃)₂, —(CH₂CH₂O)₃CH₂CH₂N(CH₃)₂, —(CH₂CH₂O)₃CH₂CH₂N(CH₃)₃Cl⁻, —(CH₂CH₂O)₅CH₂CH₂NC(O)CH₃, —(CH₂CH₂O)₈CH₂CH₂NC(O)CH₃, or —(CH₂CH₂O)₁₁CH₂CH₂NC(O)CH₃;
X3 is a direct bond, (D) arginine, (D) lysine substituted with R^{b},

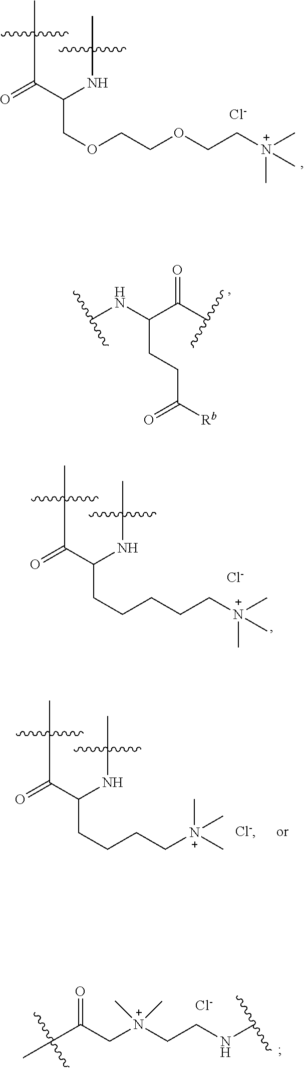

wherein:
R^{b} is

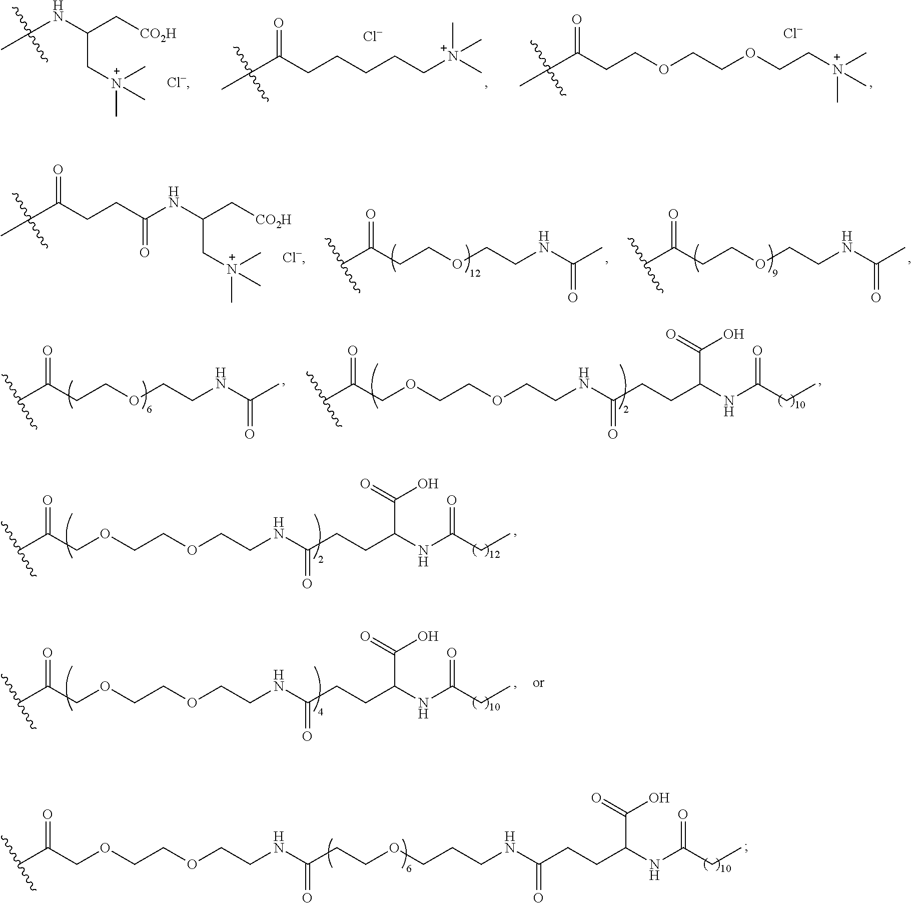

R² is H, —CH(CH₃)₂,

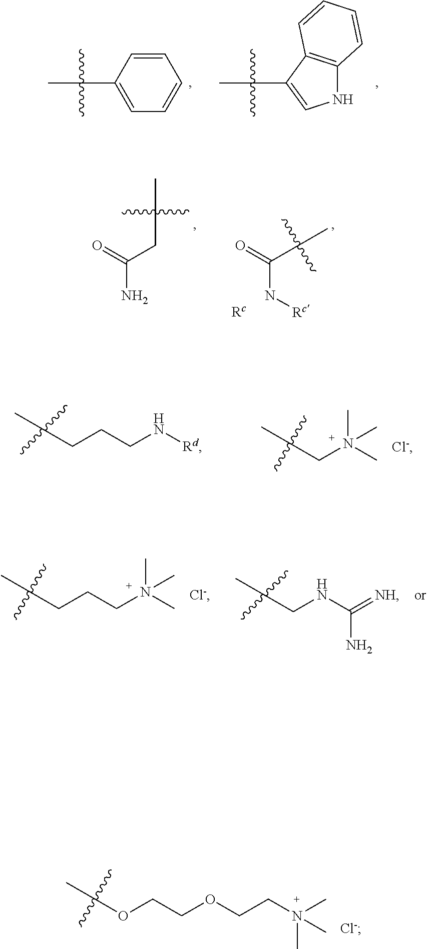

wherein
R^{c} is H, or —CH₃;
R^{c}′ is H, or —CH₃;

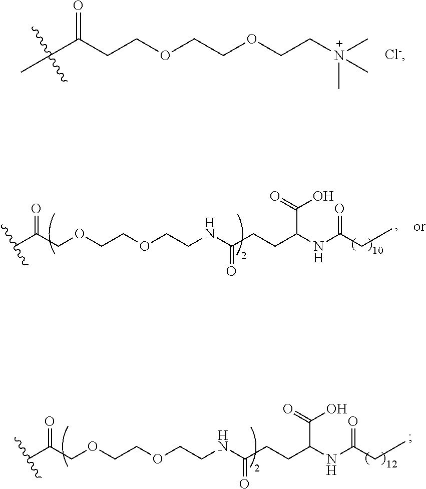

R³ is H, —CH₃, —CF₃, or

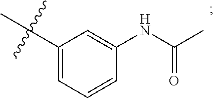 [SMILES: *c1cccc(NC(C)=O)c1]

R⁴ is H, or —CH₃;
X8 is

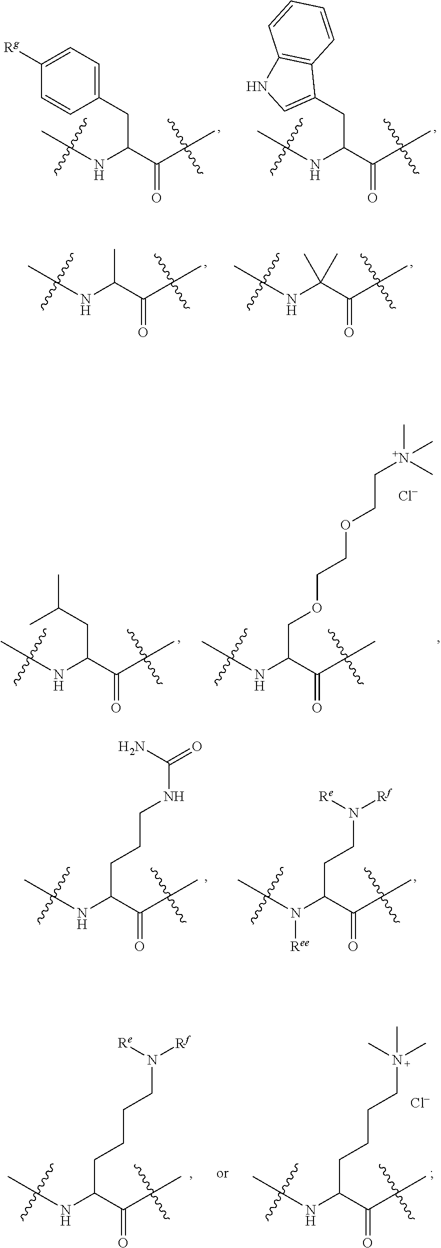

wherein:
   R^{e} is H, or —CH₃;
   R^{ee} is H;
   R^{f} is H, —CH₃, —C(CH₃)₃, —C(O)CH₃, —C(O)(CH₂CH₂O)₆CH₃, —(CH₂CH₂O)₆CH₃,

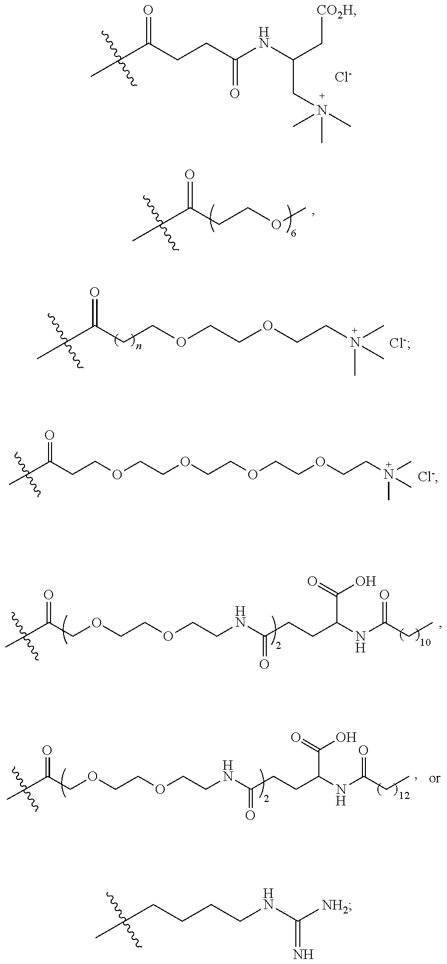

R^{g} is H, —OH, —C(O)NH₂, or —CH₂NHC(O)CH₃;
n is 0, or 1;
X12 is absent,

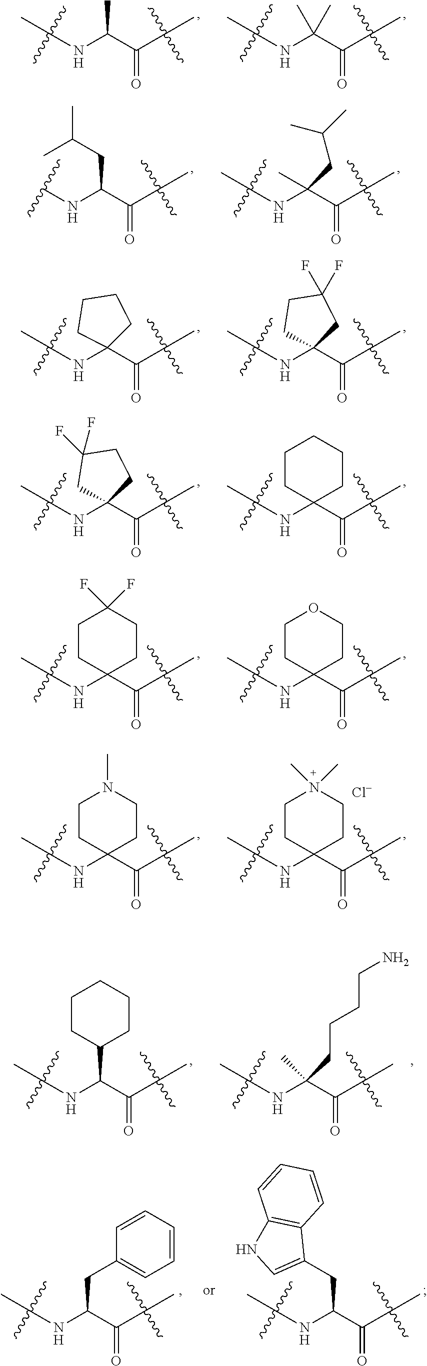

R⁶ is —CH₃, —CH₂CH₂C(O)R^{m}, —(CH₂)ₛNRⁿR^{o},

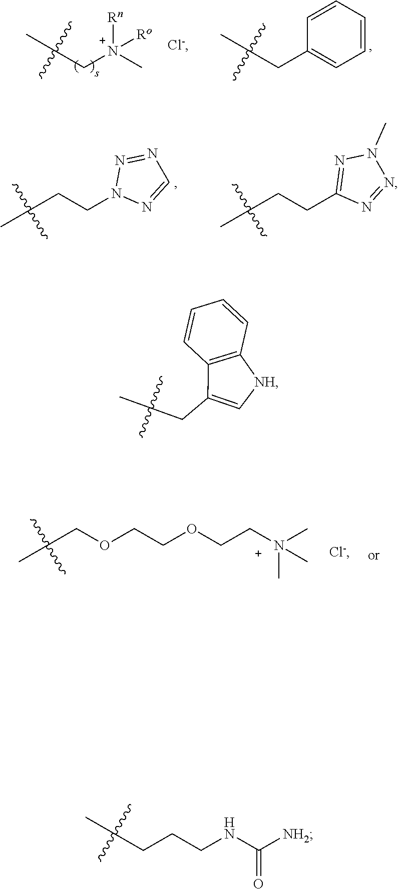

R⁷ is H, or —CH₃;
wherein:
   R^{m} is —OH, or

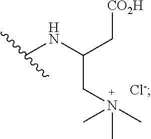

Rⁿ is H, —CH₃, —CH₂CH(CH₃)₂, —C(O)CH₃, —C(O)(CH₂CH₂O)₆CH₃, —(CH₂CH₂O)₆CH₃, —C(O)(CH₂CH₂O)ₜCH₂CH₂R^{p},

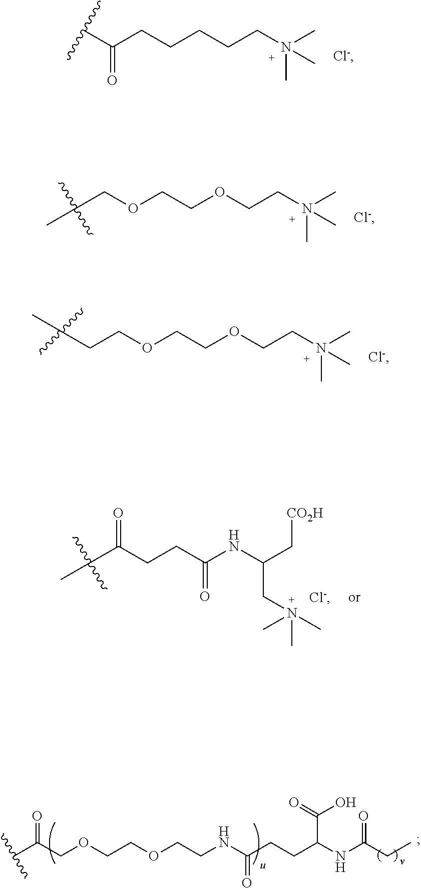

R^{o} is H, —CH₃, or —C(O)CH₃;
   wherein:
      R^{p} is NHC(O)CH₃, or N

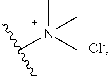

s is 1, 2, or 4;
t is 2, 6, 9, or 12;
u is 2 or 3;
v is 10 or 12;
R⁸ is —CH₂CH₂CH₂NHC(O)CH₃, —C(O)NH₂, or —C(O)NHCH₃;
X15 is absent,

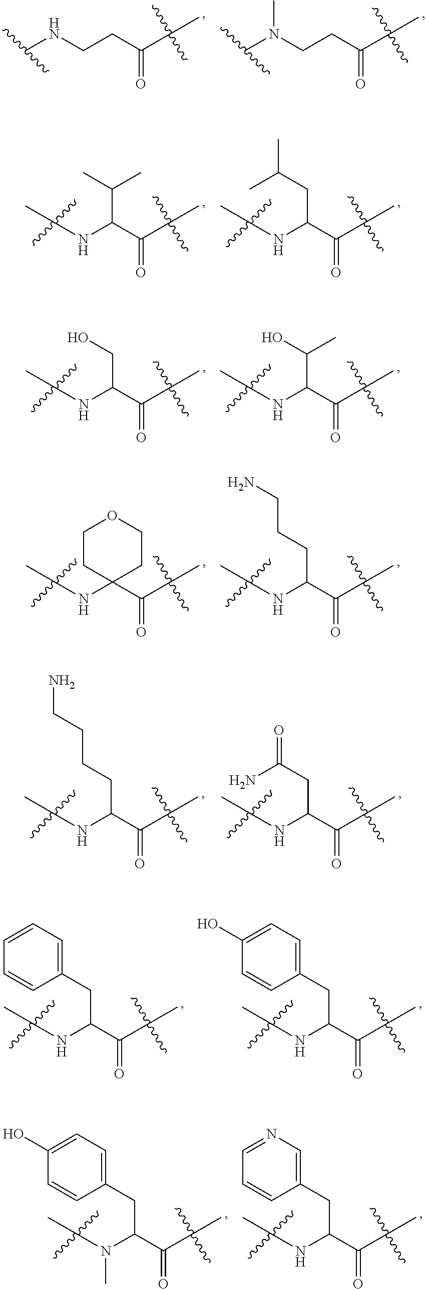

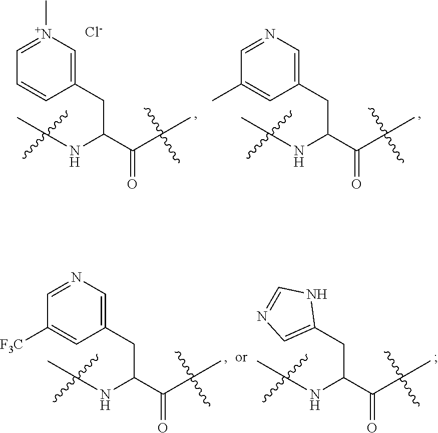 [SMILES: *NC(Cc1ccc[n+](C)c1)C(*)=O ; [Cl-] ; *NC(Cc1cncc(C)c1)C(*)=O ; *NC(Cc1cncc(C(F)(F)F)c1)C(*)=O ; *NC(Cc1cnc[nH]1)C(*)=O]

X16 is absent,

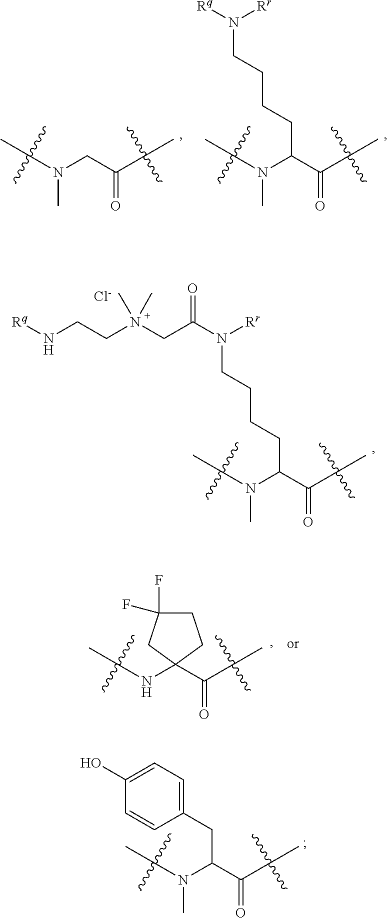

wherein:
   R^{q} is

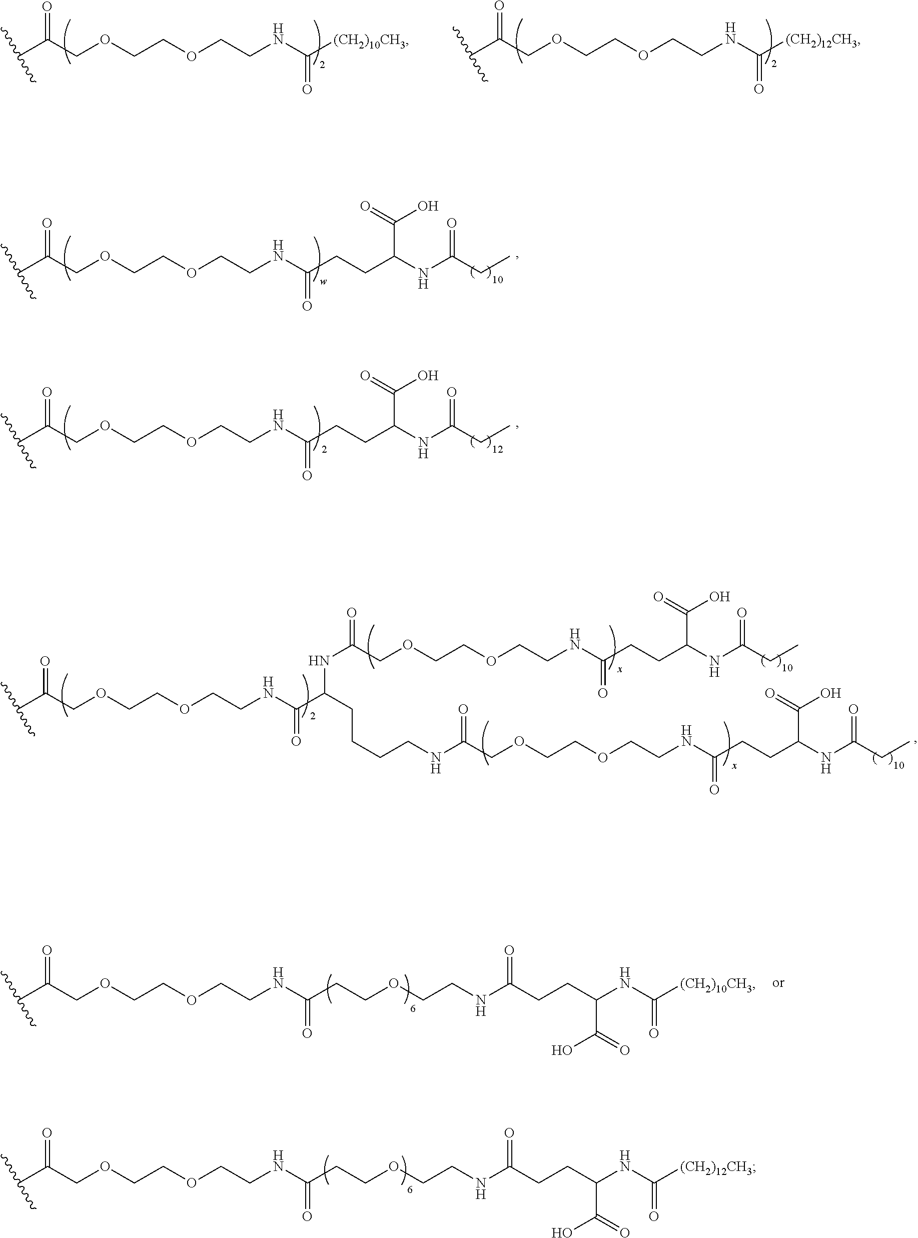

R^{r} is H;
w is 2 4, or 5;
x is 2 or 4; and
R⁹ is —NH₂, —NHCH₃, —N(CH₃)₂, —NH(CH₂CH₂O)₂CH₃, —N(CH₃)(CH₂CH₂O)₂CH₃, —NH(CH₂CH₂O)_{y}R^{s},

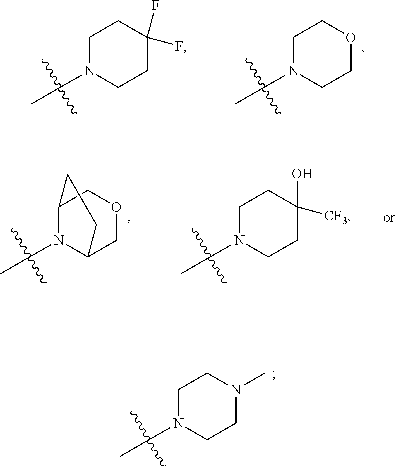

wherein:
   y is 2, 4, or 6;
R^{s} is —CH₃, or

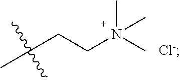

or a pharmaceutically acceptable salt thereof.

In another aspect of the invention:

4. The compound of any of aspects 1 to 3 wherein
R² is

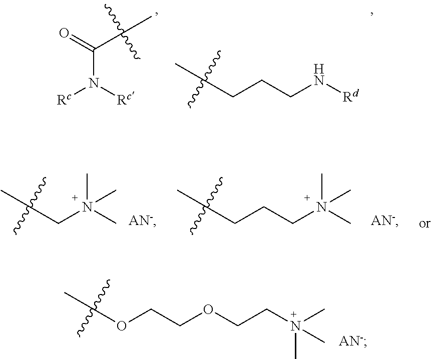

wherein:
   R^{c} is —CH₃;
   R^{c}′ is —CH₃;
   R^{d} is

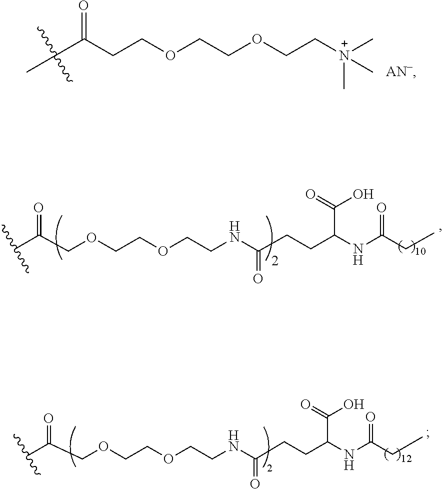

wherein:
AN⁻as above defined for A, R, X3, X12, R1, R2, R5, R6, R8, R15, R16 and R^{a}, R^{b}, R^{d}, R^{f}, R^{h}, Rⁱ, R^{j}, R^{k}, R^{m}, Rⁿ, R^{o}, R^{s} is acetate, adipate, benzoate, benzenesulfonate, citrate, decanoate, chloride, lactate, maleate, methanesulfonate, oxalate, pivalate, propionate, succinate, sulfate, tartrate, or trifluoroacetate; or a pharmaceutically acceptable salt thereof.

In another aspect of the invention:

5. A compound according to Formula (III) PGP-524 C1

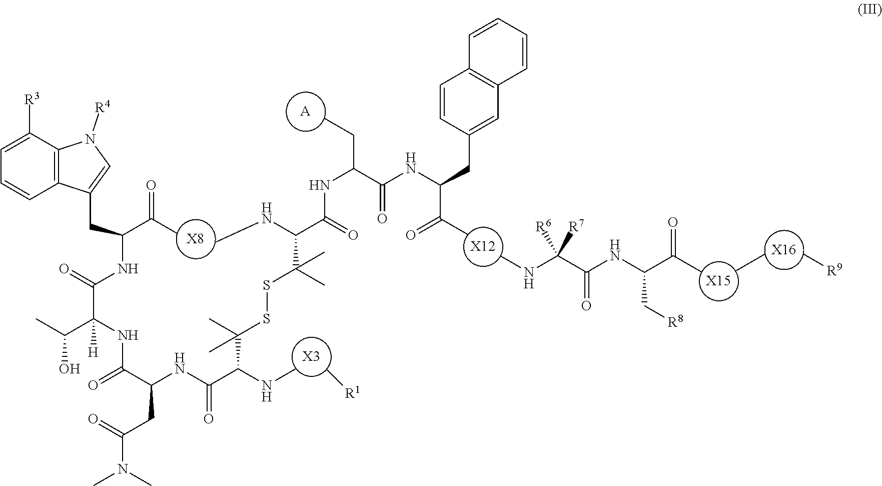

wherein:
A is

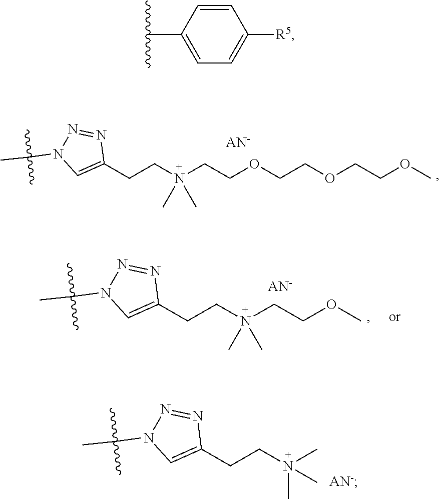

wherein:
   R⁵ is —OCHF₂, —O(CH₂)₉CO₂H,

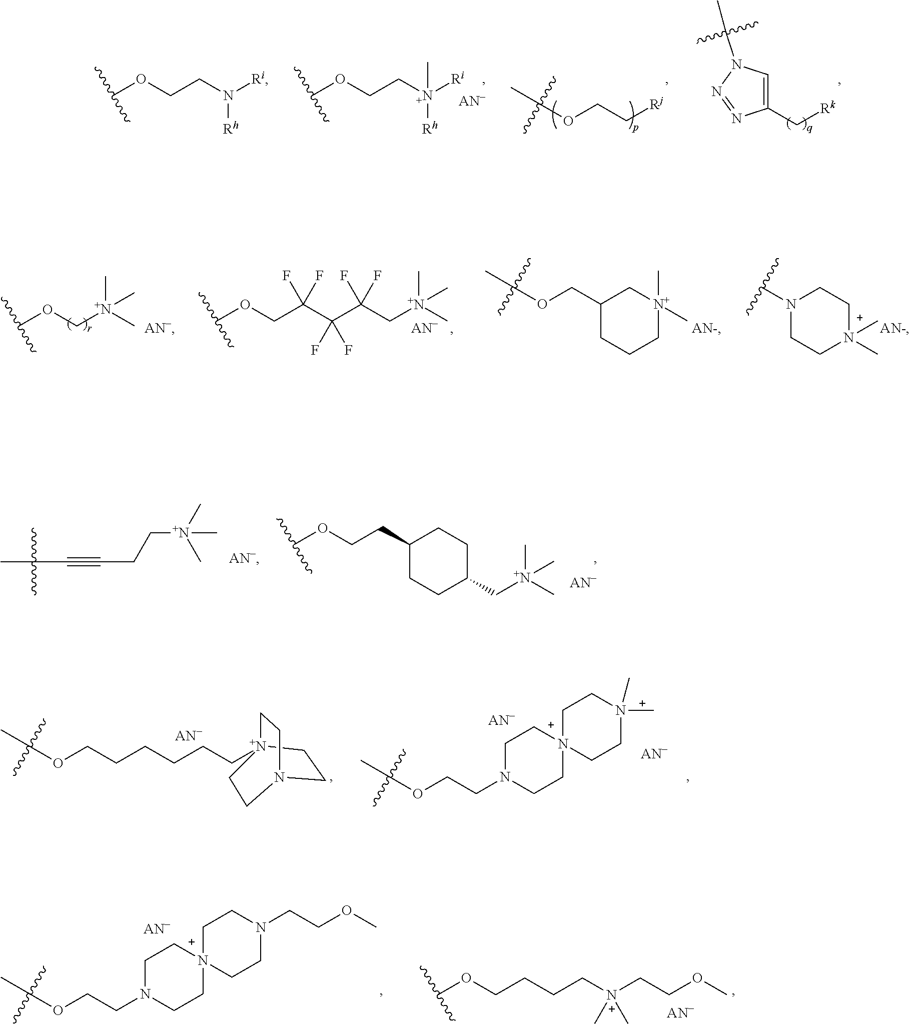

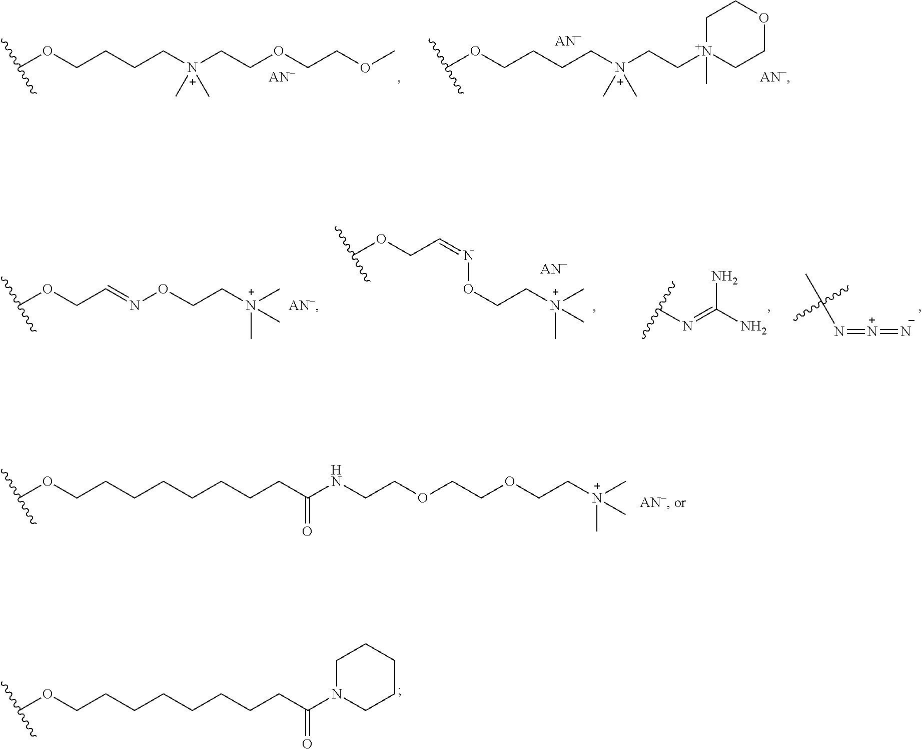

wherein:
R^{h} is H, —CH₃, —C(O)CH₃, —C(NH)NH₂, —(CH₂)₃O(CH₂)₂OCH₃, —CH₂CH₂OCH₃, —(CH₂CH₂O)₃CH₃, —(CH₂CH₂O)₆CH₃,

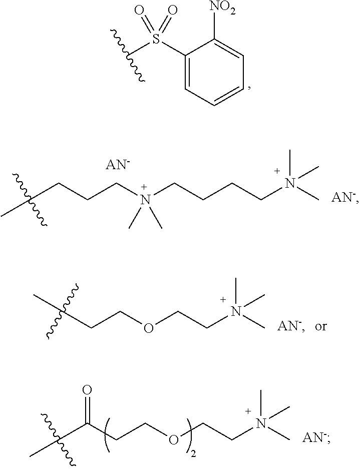

Rⁱ is H, —CH₃, —(CH₂)₃O(CH₂)₂OCH₃,

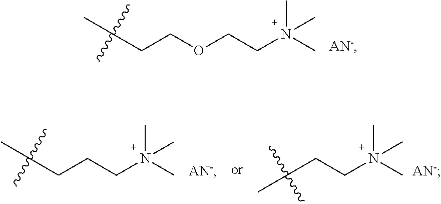

p is 1, 2 or 3;
R^{j} is

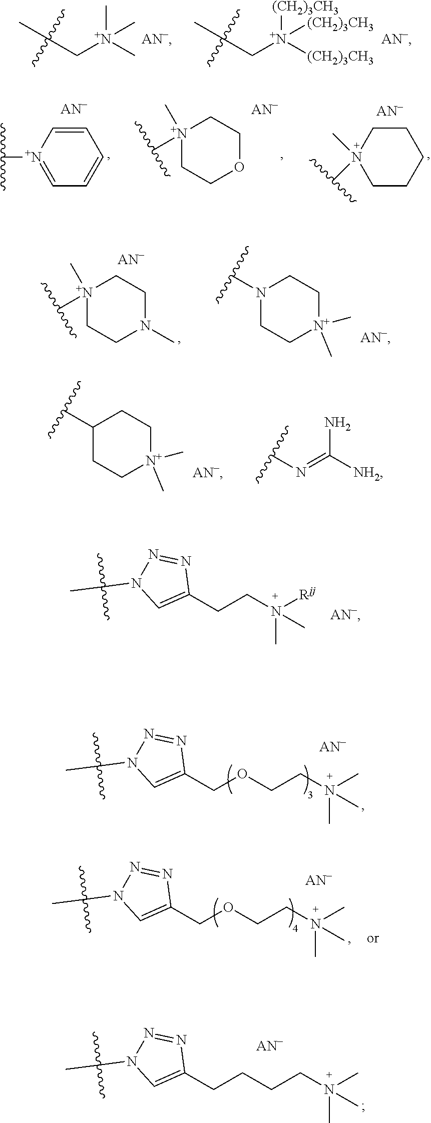

q is 1, 2, 3, 4, or 5;
R^{k} is —OCH₃

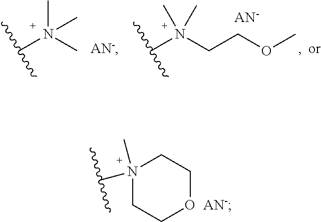

r is 3, 4, 5, 6, or 8;
wherein:
   R^{jj} is H, —(CH₂)₇CH₃, —(CH₂)₁₅CH₃, —(CH₂)₂OCH₃, or —(CH₂CH₂O)₃CH₃;
      R¹ is —C(O)C₍₁₋₂₎alkyl, —C(O)CF₃, —COCH₂CF₃, —CO(CH₂)₅N(CH₃)₃⁺AN⁻, —CH₂CH₂NHC(O)CH₂O(CH₂CH₂O)₂CH₃, —CH₂CH₂NHC(O)(CH₂CH₂O)₆CH₃,

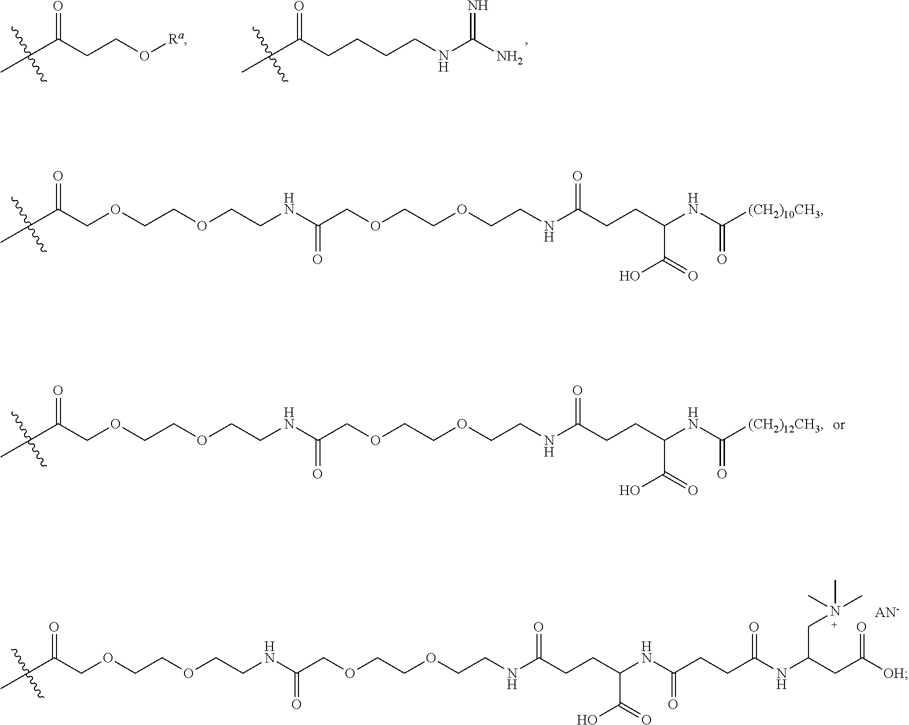

wherein:
   R^{a} is

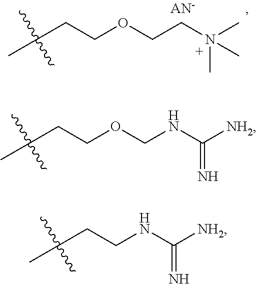

CH₂CH₂OCH₂CH₂N(CH₃)₂, —(CH₂CH₂O)₃CH₂CH₂N(CH₃)₂, —(CH₂CH₂O)₃CH₂CH₂N(CH₃)₃ ⁺AN⁻, —(CH₂CH₂O)₅CH₂CH₂NC(O)CH₃, —(CH₂CH₂O)₈CH₂CH₂NC(O)CH₃, or —(CH₂CH₂O)₁₁CH₂CH₂NC(O)CH₃;
X3 is a direct bond, (D) arginine, (D) lysine substituted with R^{b},

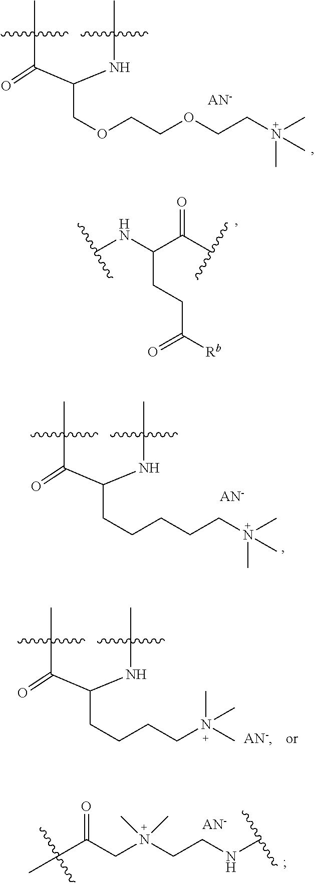

wherein:
   R^{b} is

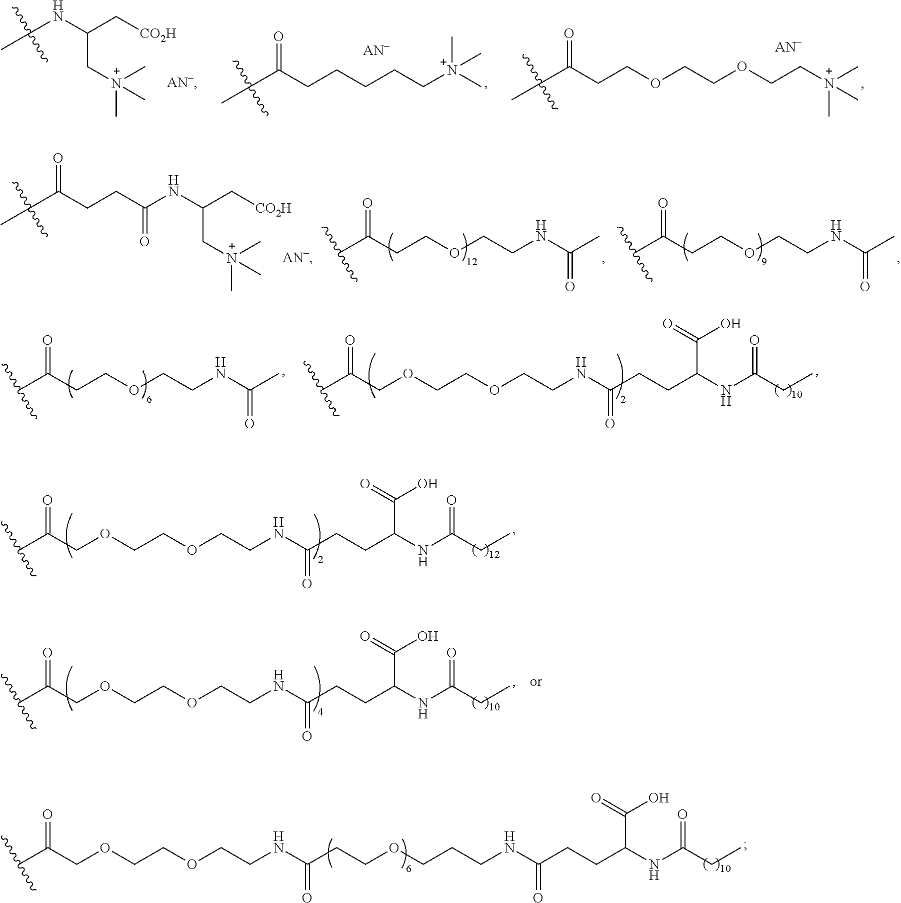

R³ is H, —CH₃, —CF₃, or

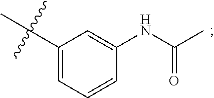 [SMILES: *c1cccc(NC(C)=O)c1]

R⁴ is H, or —CH₃
X8 is

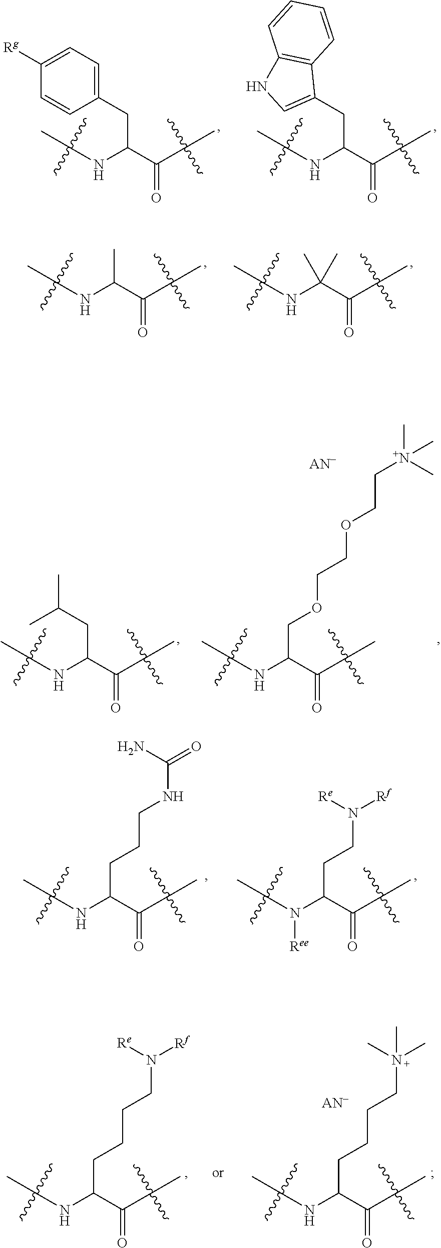

wherein:
   R^{e} is H, or —CH₃;
   R^{ee} is H;
   R^{f} is H, —CH₃, —C(CH₃)₃, —C(O)CH₃, —C(O)(CH₂CH₂O)₆CH₃, —(CH₂CH₂O)₆CH₃

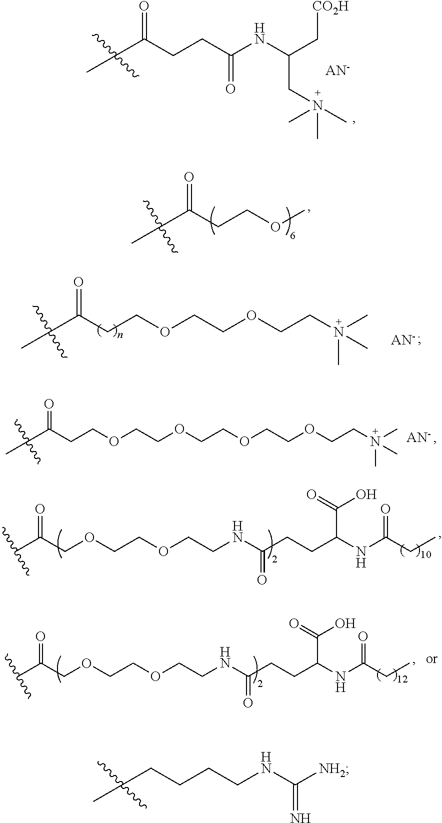

R^{g} is H, —OH, —C(O)NH₂, or —CH₂NHC(O)CH₃;
n is 0, or 1;
X12 is absent,

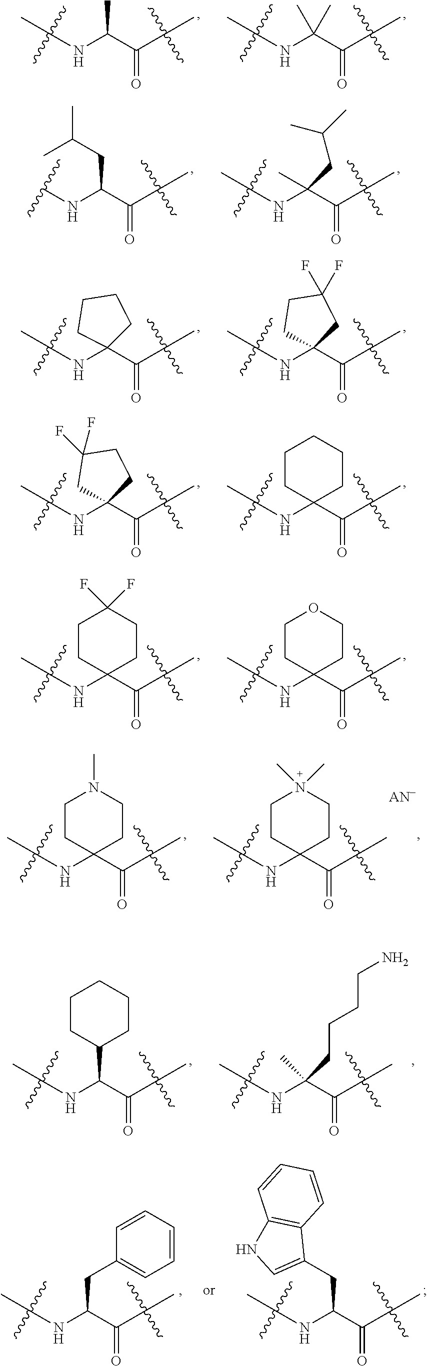

R⁶ is —CH₃, —CH₂CH₂C(O)R^{m}, —(CH₂)ₛNRⁿR^{o},

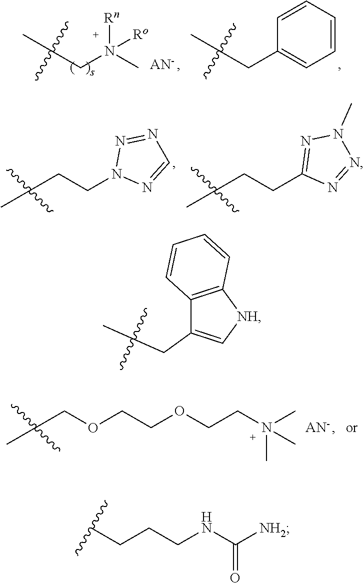

R⁷ is H, or —CH₃;
wherein:
   R^{m} is —OH, or

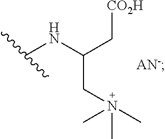

Rⁿ is H, —CH₃, —CH₂CH(CH₃)₂, —C(O)CH₃, —C(O)(CH₂CH₂O)₆CH₃, —(CH₂CH₂O)₆CH₃, —C(O)(CH₂CH₂O)ₜCH₂CH₂R^{p},

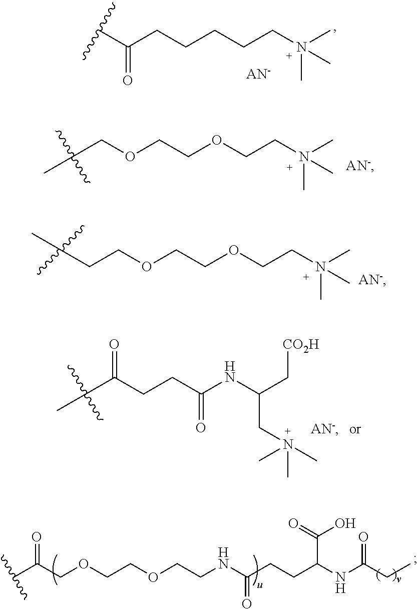

R^{o} is H, —CH₃, or —C(O)CH₃;
   wherein:
      R^{p} is NHC(O)CH₃, or

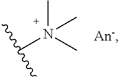

s is 1, 2, or 4;
t is 2, 6, 9, or 12;
u is 2 or 3;
v is 10 or 12;
R⁸ is —CH₂CH₂CH₂NHC(O)CH₃, —C(O)NH₂, or —C(O)NHCH₃;
X15 is absent,

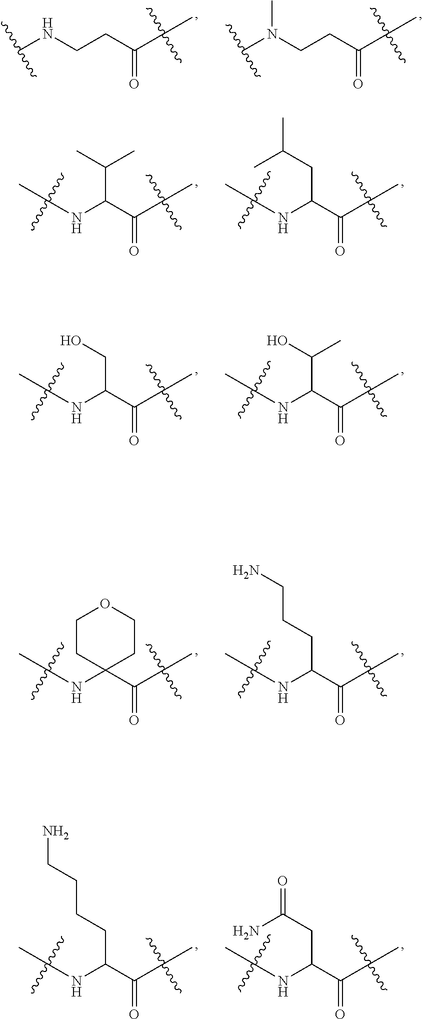

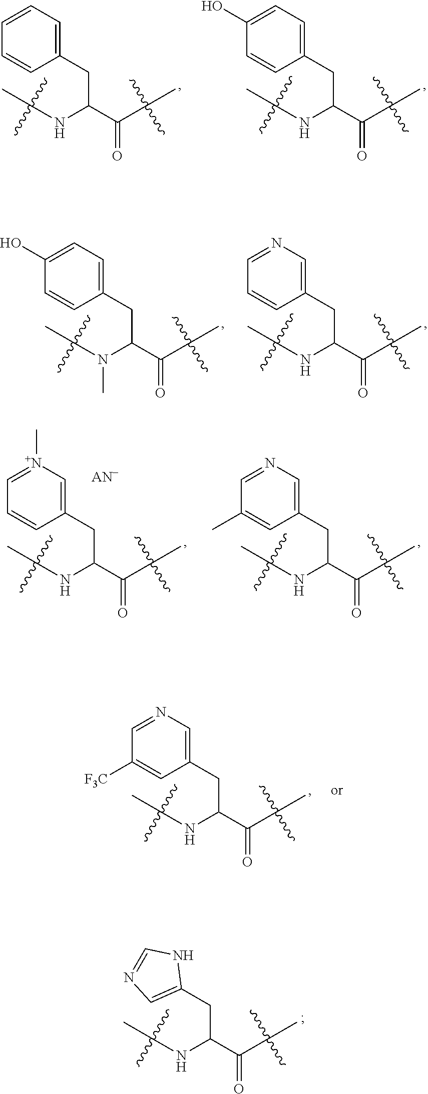

X16 is absent,

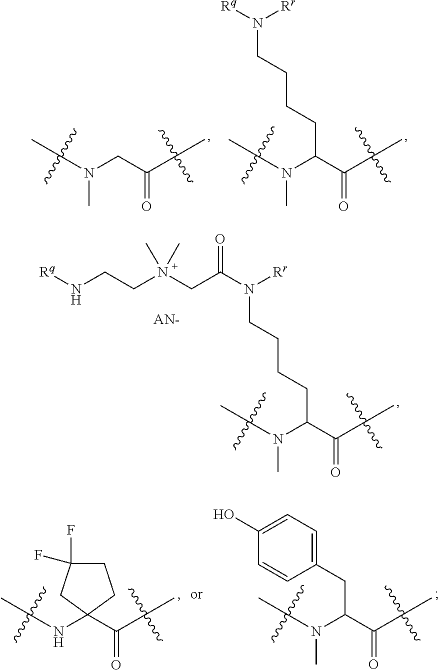

wherein:
   R^{q} is

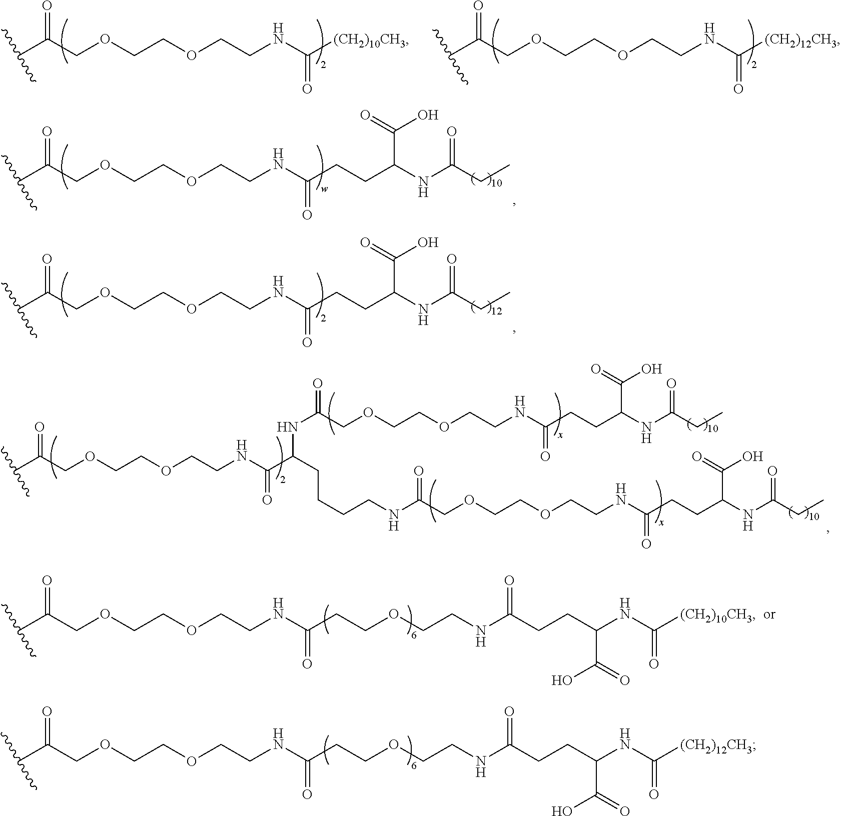

R^{r} is H;
w is 2 4, or 5;
x is 2 or 4; and
R⁹ is —NH₂, —NHCH₃, —N(CH₃)₂, —NH(CH₂CH₂O)₂CH₃, —N(CH₃)(CH₂CH₂O)₂CH₃, —NH(CH₂CH₂O)_{y}R^{S},

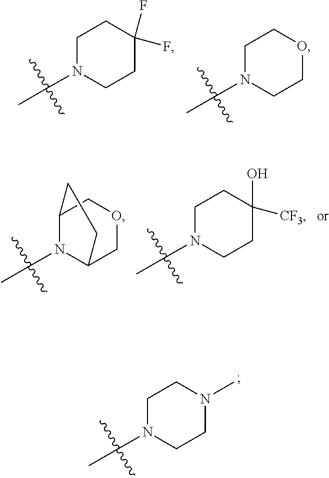

wherein:
   y is 2, 4, or 6;
   R^{s} is —CH₃, or

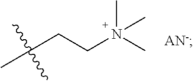

wherein:
AN⁻as above defined for A, R, X3, X12, R1, R2, R5, R6, R8, R15, R16 and R^{a}, R^{b}, R^{d}, R^{f}, R^{h}, Rⁱ, R^{j}, R^{k}, R^{m}, Rⁿ, R^{o}, R^{s} is acetate, adipate, benzoate, benzenesulfonate, citrate, decanoate, chloride, lactate, maleate, methanesulfonate, oxalate, pivalate, propionate, succinate, sulfate, tartrate, or trifluoroacetate; or
a pharmaceutically acceptable salt thereof.

In another aspect of the invention:

6. The compound of aspect 5 wherein
A is

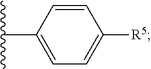

wherein:
   AN⁻as above defined for A, R, X3, X12, R1, R2, R5, R6, R8, R15, R16 and R^{a}, R^{b}, R^{d}, R^{f}, R^{h}, Rⁱ, R^{j}, R^{k}, R^{m}, Rⁿ, R^{o}, R^{s} is acetate, adipate, benzoate, benzenesulfonate, citrate, decanoate, chloride, lactate, maleate, methanesulfonate, oxalate, pivalate, propionate, succinate, sulfate, tartrate, or trifluoroacetate; or
a pharmaceutically acceptable salt thereof.

In another aspect of the invention:

7. The compound of aspect 5 wherein
A is

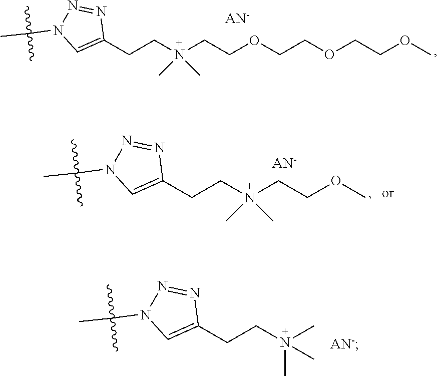

wherein:
   AN⁻as above defined for A, R, X3, X12, R1, R2, R5, R6, R8, R15, R16 and R^{a}, R^{h}, R^{d}, R^{f}, R^{h}, Rⁱ, R^{j}, R^{k}, R^{m}, Rⁿ, R^{o}, R^{s} is acetate, adipate, benzoate, benzenesulfonate, citrate, decanoate, chloride, lactate, maleate, methanesulfonate, oxalate, pivalate, propionate, succinate, sulfate, tartrate, or trifluoroacetate; or
a pharmaceutically acceptable salt thereof.

In another aspect of the invention:

8. The compound of aspect 7 wherein
X8 is

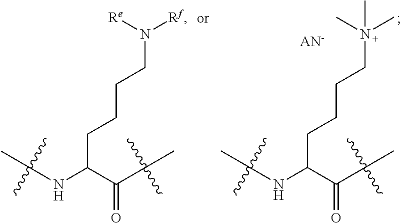

wherein:
   AN⁻as above defined for A, R, X3, X12, R1, R2, R5, R6, R8, R15, R16 and R^{a}, R^{h}, R^{d}, R^{f}, R^{h}, Rⁱ, R^{j}, R^{k}, R^{m}, Rⁿ, R^{o}, R^{s} is acetate, adipate, benzoate, benzenesulfonate, citrate, decanoate, chloride, lactate, maleate, methanesulfonate, oxalate, pivalate, propionate, succinate, sulfate, tartrate, or trifluoroacetate; or
a pharmaceutically acceptable salt thereof.

In another aspect of the invention:

9. The compound of aspect 5 selected from
cPEG3aCO-Pen(3)—N(N(Me)2)-T-7MeW—K(NMeAc)-Pen(3)-TMAPF-2Nal-THP-K(NMeAc)—N-3Pya-Sar-CO(NHPEG3a);
MeCO-Pen(3)—N(N(Me)2)-T-7MeW—K(NMeAc)-Pen(3)-TMAPF-2Nal-THP-K(NMeAc)—N-3Pya-Sar-CO(NHPEG3a);
cPEG3aCO-Pen(3)—N(N(Me)2)-T-7MeW—K(NMeAc)-Pen(3)-APEG3F-2Nal-THP-A-N-3Pya-Sar-CON(Me)2;
cPEG3aCO-Pen(3)—N(N(Me)2)-T-7MeW—K(NMeAc)-Pen(3)-TMAPF-2Nal-THP-A-N-3Pya-Sar-CON(Me)2;
MeCO-Pen(3)—N(N(Me)2)-T-7MeW-Dab(NMeAc)-Pen(3)-AEF-2Nal-THP-AIB-N-3Pya-Sar-CON(Me)2;
cPEG3aCO-Pen(3)—N(N(Me)2)-T-7MeW-AIB-Pen(3)-AEF-2Nal-THP-AIB-N-3Pya-Sar-CON(Me)2;
MeCO-Pen(3)—N(N(Me)2)-T-7MeW—K(NMeAc)-Pen(3)-TMAPF-2Nal-THP-APEG2Ser-N-3Pya-Sar-CON(Me)2;
MeCO-Pen(3)—N(N(Me)2)-T-7MeW-APEG2Ser(S*)-Pen(3)-APEG3F-2Nal-THP-APEG2Ser(S*)—N-3Pya-Sar-CON(Me)2;
MeCO-Pen(3)—N(N(Me)2)-T-7MeW-APEG2Ser(R*)-Pen(3)-APEG3F-2Nal-THP-APEG2Ser(S*)—N-3Pya-Sar-CON(Me)2;
MeCO-Pen(3)—N(N(Me)2)-T-7MeW—K(NMeAc)-Pen(3)-APEG3F-2Nal-THP-APEG2Ser(S*)—N-3Pya-Sar-CON(Me)2;
cPEG3aCO-Pen(3)—N(N(Me)2)-T-7MeW—K(NMeAc)-Pen(3)-TMAPF-2Nal-THP-Cit-N-3Pya-Sar-CON(Me)2;
cPEG3aCO-Pen(3)—N(N(Me)2)-T-7MeW—K(NMeAc)-Pen(3)-AEF-2Nal-diFCpx-Dab(NMeAc)—N-3Pya-Sar-CON(Me)2;
cPEG3aCO-Pen(3)—N(N(Me)2)-T-7MeW—K(NMeAc)-Pen(3)-AEF-2Nal-diFCpx-Dab(NMeAc)—N-3Pya-Sar-CON(Me)2;
cPEG3aCO-Pen(3)—N(N(Me)2)-T-7MeW—K(Ac)-Pen(3)-TMAPF-2Nal-THP-Dab(NMeAc)—N-3Pya-Sar-CON(Me)2;
cPEG3aCO-Pen(3)—N(N(Me)2)-T-7MeW—K(NMeAC)-Pen(3)-TMAPF-2Nal-THP-Dab(NMeAc)—N-3Pya-Sar-CON(Me)2;
MeCO-Pen(3)—N(N(Me)2)-T-7MeW-Dab(NMecarn)-Pen(3)-APEG3F-2Nal-THP-Dab(NMeAc)—N-3Pya-Sar-CON(Me)2;
MeCO-k(d)-Pen(3)—N(N(Me)2)-T-7MeW—K(NMeAc)-Pen(3)-APEG3F-2Nal-THP-Dab(NMeAc)—N-3Pya-Sar-CON(Me)2;
cPEG3aCO-Pen(3)—N(N(Me)2)-T-7MeW—K(NMeAc)-Pen(3)-APEG3F-2Nal-THP-Dab(NMeAc)—N-3Pya-Sar-CON(Me)2;
cPEG3aCO-Pen(3)—N(N(Me)2)-T-7MeW—K(NMeAc)-Pen(3)-APEG3F-2Nal-THP-Dab(NMeAc)—N-3Pya-Sar-CON(Me)2;
mPEG3CO-SP6-Pen(3)—N(N(Me)2)-T-7MeW—K(NMeAc)-Pen(3)-APEG3F-2Nal-THP-Dab(NMeAc)—N-3Pya-Sar-CON(Me)2;
cPEG3aCO-Pen(3)—N(N(Me)2)-T-7MeW-Dab(NMecPEG3a)-Pen(3)-AEF-2Nal-THP-Dab(NMeAc)—N-3Pya-Sar-CON(Me)2;
cPEG3aCO-Pen(3)—N(N(Me)2)-T-7MeW—K(NMeAc)-Pen(3)-AEF-2Nal-THP-Dab(NMeAc)—N-3Pya-Sar-CON(Me)2;
cPEG5aCO-Pen(3)—N(N(Me)2)-T-7MeW-Q(N(Me)2)-Pen(3)-AEF-2Nal-THP-Dab(NMeAc)—N-3Pya-Sar-CON(Me)2;
MeCO-Pen(3)—N(N(Me)2)-T-7MeW-Dab(NMeAc)-Pen(3)-AEF-2Nal-THP-Dab(NMeAc)—N-3Pya-Sar-CON(Me)2;
cPEG3aCO-Pen(3)—N(N(Me)2)-T-7MeW-Dab(NMeAc)-Pen(3)-AEF-2Nal-THP-Dab(NMeAc)—N-3Pya-Sar-CON(Me)2;
cPEG3aCO-Pen(3)—N(N(Me)2)-T-7MeW-Q(N(Me)2)-Pen(3)-AEF-2Nal-THP-Dab(NMeAc)—N-3Pya-Sar-CON(Me)2;
cPEG5aCO-Pen(3)—N(N(Me)2)-T-7MeW-Dab(NMeAc)-Pen(3)-AEF-2Nal-THP-Dab(NMeAc)—N-3Pya-Sar-CON(Me)2;
MeCO-Pen(3)—N(N(Me)2)-T-7MeW-Dab(NMecarn)-Pen(3)-APEG3F-2Nal-THP-Dab(NMecarn)-N-3Pya-Sar-CON(Me)2;
MeCO-Pen(3)—N(N(Me)2)-T-7MeW-Dab(NMeCOmPEG6)-Pen(3)-AEF-2Nal-THP-Dab(NMeCOmPEG6)—N-3Pya-Sar-CON(Me)2;
cPEG5aCO-Pen(3)—N(N(Me)2)-T-7MeW-Dab(NMecPEG2a)-Pen(3)-AEF-2Nal-THP-Dab(NMecPEG2a)—N-3Pya-Sar-CON(Me)2;
MeCO-Pen(3)—N(N(Me)2)-T-7MeW-Dab(NMecPEG3a)-Pen(3)-AEF-2Nal-THP-Dab(NMecPEG3a)—N-3Pya-Sar-CON(Me)2;
5cpaCO-Pen(3)—N(N(Me)2)-T-7MeW—K(NMeAc)-Pen(3)-AEF(N(Me)2)-2Nal-THP-Dap(Ac)—N-3Pya-Sar-CON(Me)2;
MeCO-Pen(3)—N(N(Me)2)-T-7MeW—K(NMeAc)-Pen(3)-AEF(N(Me)2)-2Nal-THP-Dap(Ac)—N-3Pya-Sar-CON(Me)2;
cPEG3aCO-Pen(3)—N(N(Me)2)-T-7MeW—K(NMeAc)-Pen(3)-APEG3F-2Nal-THP-Dap(NMeAc)—N-3Pya-Sar-CON(Me)2;
cPEG3aCO-Pen(3)—N(N(Me)2)-T-7MeW—K(Ac)-Pen(3)-TMAPF-2Nal-THP-Dap(NMeAc)—N-3Pya-Sar-CON(Me)2;
cPEG3aCO-Pen(3)—N(N(Me)2)-T-7MeW—K(NMeAc)-Pen(3)-APEG3F-2Nal-THP-E-N-3Pya-Sar-CON(Me)2;
cPEG3aCO-Pen(3)—N(N(Me)2)-T-7MeW-Dab(NMeAc)-Pen(3)-AEF-2Nal-THP-E-N-3Pya-Sar-CONH2;
cPEG3aCO-Pen(3)—N(N(Me)2)-T-7MeW—K(NMeAc)-Pen(3)-TMAPF-2Nal-THP-E-N-3Pya-Sar-CON(Me)2;
MeCO-hk(Me)₃-Pen(3)—N(N(Me)2)-T-7MeW—K(NMeAc)-Pen(3)-AEF-2Nal-THP-E-N-3Pya-Sar-CON(Me)2;
MeCO-Pen(3)—N(N(Me)2)-T-7MeW-Dab(NMeCOmPEG6)-Pen(3)-AEF-2Nal-THP-E-N-3Pya-Sar-CON(Me)2;
MeCO-Pen(3)—N(N(Me)2)-T-7MeW-Dab(NMeCarn)-Pen(3)-AEF-2Nal-THP-E-N-3Pya-Sar-CON(Me)2;
cPEG5aCO-Pen(3)—N(N(Me)2)-T-7MeW-Dab(NMecPEG5a)-Pen(3)-AEF-2Nal-THP-E-N-3Pya-Sar-CON(Me)2;
mPEG6CO-SP6-Pen(3)—N(N(Me)2)-T-7MeW-Dab(NMecPEG5a)-Pen(3)-AEF-2Nal-THP-E-N-3Pya-Sar-CON(Me)2;
MeCO-Pen(3)—N(N(Me)2)-T-7MeW-Dab(NMecarn)-Pen(3)-AEF-2Nal-THP-E-N-3Pya-Sar-CON(Me)2;
MeCO-Pen(3)—N(N(Me)2)-T-7MeW-Dab(NMecPEG2a)-Pen(3)-AEF-2Nal-THP-E-N-3Pya-Sar-CON(Me)2;
MeCO-Pen(3)—N(N(Me)2)-T-7MeW-Dab(NMeAc)-Pen(3)-AEF-2Nal-THP-E-N-3Pya-Sar-CON(Me)2;
MeCO-Pen(3)—N(N(Me)2)-T-7MeW-Q(N(Me)2)-Pen(3)-AEF(N(Me)2)-2Nal-THP-E-N-3Pya-Sar-CON(Me)2;
MeCO-Pen(3)—N(N(Me)2)-T-7MeW—K(NMeAc)-Pen(3)-AEF(N(Me)2)-2Nal-THP-E-N-3Pya-Sar-CON(Me)2;
MeCO-k(Me)₃-Pen(3)—N(N(Me)2)-T-7MeW—K(NMeAc)-Pen(3)-AEF(N(Me)2)-2Nal-THP-E-N-3Pya-Sar-CON(Me)2;
CF3CO-Pen(3)—N(N(Me)2)-T-7MeW—K(NMeAc)-Pen(3)-AEF(N(Me)2)-2Nal-THP-E-N-3Pya-Sar-CON(Me)2;
5cpaCO-Pen(3)—N(N(Me)2)-T-7MeW-Q(N(Me)2)-Pen(3)-AEF(N(Me)2)-2Nal-THP-E-N-3Pya-Sar-CON(Me)2;
CF3CO—K(Me)₃-Pen(3)—N(N(Me)2)-T-7MeW—K(NMeAc)-Pen(3)-AEF(N(Me)2)-2Nal-THP-E-N-3Pya-Sar-CON(Me)2;
cPEG3aCO-Pen(3)—N(N(Me)2)-T-7MeW—K(NMeAc)-Pen(3)-APEG3F-2Nal-THP—F—N-3Pya-Sar-CON(Me)2;
cPEG3aCO-Pen(3)—N(N(Me)2)-T-7MeW—K(NMeAc)-Pen(3)-APEG3F-2Nal-THP—K(Ac)—N-3Pya-Sar-CON(Me)2;
MeCO-Pen(3)—N(N(Me)2)-T-7MeW—K(Me)₃-Pen(3)-AEF(N(Me)2)-2Nal-THP-K(Me)₃—N-3Pya-Sar-CON(Me)2;
cPEG3aCO-Pen(3)—N(N(Me)2)-T-7MeW—K(NMeAc)-Pen(3)-TMAPF-2Nal-Achx-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-Acpx-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-Aib-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-Chg-K(NMeAc)—N-3Pya-Sar-CON(Me)2;
cPEG3aCO-Pen(3)—N(N(Me)2)-T-7MeW—K(NMeAc)-Pen(3)—Y(OTz1TMA4)-2Nal-THP-K(NMeAc)—N-3Pya-Sar-CON(Me)2;
cPEG3aCO-Pen(3)—N(N(Me)2)-T-7(3NAcPh)W—K(NMeAc)-Pen(3)-MMoEF-2Nal-THP-K(NMeAc)—N-3Pya-Sar-CON(Me)2;
cPEG3aCO-Pen(3)—N(N(Me)2)-T-7MeW-Dap(NMeAc)-Pen(3)-TMAPF-2Nal-THP-K(NMeAc)—N-3Pya-Sar-CON(Me)2;
cPEG2gCO-Pen(3)—N(N(Me)2)-T-7MeW—K(NMeAc)-Pen(3)-APEG3F-2Nal-THP-K(NMeAc)—N-3Pya-Sar-CON(Me)2;
cPEG3gCO-Pen(3)—N(N(Me)2)-T-7MeW—K(NMeAc)-Pen(3)-APEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-GPEG3F-2Nal-THP-K(NMeAc)—N-3Pya-Sar-CON(Me)2;
4cpgCO-Pen(3)—N(N(Me)2)-T-7MeW—K(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(PEG2PEG2gEC14)-Pen(3)-TMAPF-2Nal-THP-K(NMeAc)—N-3Pya-Sar-CON(Me)2;
cPEG3aCO-Pen(3)—N(N(Me)2)-T-7MeW—K(4cpg)-Pen(3)-TMAPF-2Nal-THP-K(NMeAc)—N-3Pya-Sar-CON(Me)2;
cPEG3gCO-Pen(3)—N(N(Me)2)-T-7MeW—K(NMeAc)-Pen(3)-TMAPF-2Nal-THP-K(NMeAc)—N-3Pya-Sar-CON(Me)2;
cPEG2gCO-Pen(3)—N(N(Me)2)-T-7MeW—K(NMeAc)-Pen(3)-TMAPF-2Nal-THP-K(NMeAc)—N-3Pya-Sar-CON(Me)2;
C14gEPEG2PEG2CO-k(PEG2PEG2gEC14)-Pen(3)—N(N(Me)2)-T-7MeW-Dab(NMeAc)-Pen(3)-TMAPF-2Nal-THP-K(NMeAc)—N-3Pya-Sar-CON(Me)2;
C12gEPEG2PEG2CO-k(PEG2PEG2gEC12)-Pen(3)—N(N(Me)2)-T-7MeW-Dab(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(cPEG3a)-Pen(3)-TMAPF-2Nal-THP-K(NMeAc)—N-3Pya-Sar-CON(Me)2;
cPEG3aCO-k(PEG2PEG2gEC12)-Pen(3)—N(N(Me)2)-T-7MeW—K(PEG2PEG2gEC12)-Pen(3)-TMAPF-2Nal-THP-K(NMeAc)—N-3Pya-Sar-CON(Me)2;
cPEG3aCO-k(PEG2PEG2gEC14)-Pen(3)—N(N(Me)2)-T-7MeW—K(NMeAc)-Pen(3)-TMAPF-2Nal-THP-K(NMeAc)—N-3Pya-Sar-CON(Me)2;
cPEG3aCO-k(PEG2PEG2gEC12)-Pen(3)—N(N(Me)2)-T-7MeW—K(NMeAc)-Pen(3)-TMAPF-2Nal-THP-K(NMeAc)—N-3Pya-Sar-CON(Me)2;
C14gEPEG2PEG2CO-APEG2Ser(S*)-Pen(3)—N(N(Me)2)-T-7MeW—K(PEG2PEG2gEC14)-Pen(3)-APEG3F-2Nal-THP-K(NMeAc)—N-3Pya-Sar-CON(Me)2;
C12gEPEG2PEG2CO-APEG2Ser(S*)-Pen(3)—N(N(Me)2)-T-7MeW—K(PEG2PEG2gEC12)-Pen(3)-APEG3F-2Nal-THP-K(NMeAc)—N-3Pya-Sar-CON(Me)2;
C14gEPEG2PEG2CO-APEG2Ser(S*)-Pen(3)—N(N(Me)2)-T-7MeW—K(NMeAc)-Pen(3)-APEG3F-2Nal-THP-K(NMeAc)—N-3Pya-Sar-CON(Me)2;
C12gEPEG2PEG2CO-APEG2Ser(S*)-Pen(3)—N(N(Me)2)-T-7MeW—K(NMeAc)-Pen(3)-APEG3F-2Nal-THP-K(NMeAc)—N-3Pya-Sar-CON(Me)2;
cPEG3aCO-Pen(3)—N(N(Me)2)-T-7MeW-Dab(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW-Cit-Pen(3)-TMAPF-2Nal-THP-K(NMeAc)—N-3Pya-Sar-CON(Me)2;
MeCO-r-Pen(3)—N(N(Me)2)-T-7MeW—K(NMeAc)-Pen(3)-TMAPF-2Nal-THP-K(NMeAc)—N-3Pya-Sar-CON(Me)2;
MeCO-hk(Me)₃-Pen(3)—N(N(Me)2)-T-7MeW—K(NMeAc)-Pen(3)-TMAPF-2Nal-THP-K(NMeAc)—N-3Pya-Sar-CON(Me)2;
5cpaCO-Pen(3)—N(N(Me)2)-T-7MeW—K(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(NMeAc)-Pen(3)-hFTMAPF-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4Tz1DMA4mPEG)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4Tz1MMo4)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4Tz1TMA5)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4Tz1TMA3)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4Tz1TMA2)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4Tz1TMA1)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4N3)-2Nal-THP-K(NMeAc)—N-3Pya-Sar-CON(Me)2;
cPEG3aCO-Pen(3)—N(N(Me)2)-T-7MeW—K(Me)₃-Pen(3)-TMAPF-2Nal-THP-K(NMeAc)—N-3Pya-Sar-CON(Me)2;
C14gEPEG2PEG2CO-hk(Me)₃-Pen(3)—N(N(Me)2)-T-7MeW—K(PEG2PEG2gEC14)-Pen(3)-TMAPF-2Nal-THP-K(NMeAc)—N-3Pya-Sar-CON(Me)2;
C14gEPEG2PEG2CO-hk(Me)₃-Pen(3)—N(N(Me)2)-T-7MeW—K(NMeAc)-Pen(3)-TMAPF-2Nal-THP-K(NMeAc)—N-3Pya-Sar-CON(Me)2;
C12gEPEG2PEG2CO-hk(Me)₃-Pen(3)—N(N(Me)2)-T-7MeW—K(PEG2PEG2gEC12)-Pen(3)-TMAPF-2Nal-THP-K(NMeAc)—N-3Pya-Sar-CON(Me)2;
C12gEPEG2PEG2CO-hk(Me)₃-Pen(3)—N(N(Me)2)-T-7MeW—K(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(N(Me)2)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF-2Nal-THP-K(NMeAc)—N-3Pya-Sar-CON(Me)2;
MeCO-APEG2Ser(S*)-Pen(3)—N(N(Me)2)-T-7MeW—K(NMeAc)-Pen(3)-APEG3F-2Nal-THP-K(NMeAc)—N-3Pya-Sar-CON(Me)2;
MeCO-APEG2Ser(R*)-Pen(3)—N(N(Me)2)-T-7MeW—K(NMeAc)-Pen(3)-APEG3F-2Nal-THP-K(NMeAc)—N-3Pya-Sar-CON(Me)2;
cPEG3aCO-Pen(3)—N(N(Me)2)-T-7MeW-Q(N(Me)2)-Pen(3)-TMAPF-2Nal-THP-K(NMeAC)—N-3Pya-Sar-CON(Me)2;
cPEG3aCO-Pen(3)—N(N(Me)2)-T-7MeW—K(NMeAc)-Pen(3)—Y(OZOXIMECh)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OEOXIMECh)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4G)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4Tz1MME)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4Tz1TMA4)-2Nal-THP-K(NMeAc)—N-3Pya-Sar-CON(Me)2;
cPEG3aCO-Pen(3)—N(N(Me)2)-T-7MeW—K(NMeAc)-Pen(3)-dFPPEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-NPyPEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-TMA8F-2Nal-THP-K(NMeAc)—N-3Pya-Sar-CON(Me)2;
cPEG3aCO-Pen(3)—N(N(Me)2)-T-7(3NAcPh)W—K(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(NMeAc)-Pen(3)-TMA6F-2Nal-THP-K(NMeAc)—N-3Pya-Sar-CON(Me)2;
cPEG3aCO-Pen(3)—N(N(Me)2)-T-7MeW—K(NMeAc)-Pen(3)-Diazabiclyclooctane6F-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OTzlChC16)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OTzlC1 aC8)-2Nal-THP-K(NMeAc)—N-3Pya-Sar-CON(Me)2;
cPEG3aCO-Pen(3)—N(N(Me)2)-T-7MeW—K(NMeAc)-Pen(3)-TzlChmPEG3-2Nal-THP-K(NMeAc)—N-3Pya-Sar-CON(Me)2;
cPEG3aCO-Pen(3)—N(N(Me)2)-T-7MeW—K(NMeAc)-Pen(3)-TzlChmPEG-2Nal-THIP-K(NMeAc)—N-3Pya-Sar-CON(Me)2;
cPEG3aCO-Pen(3)—N(N(Me)2)-T-7MeW—K(NMeAc)-Pen(3)-Tzl(Ch)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OTzlCh)-2Nal-THIP-K(NMeAc)—N-3Pya-Sar-CON(Me)2;
cPEG3aCO-Pen(3)—N(N(Me)2)-T-7MeW—K(NMeAc)-Pen(3)—Y(OTzlChmPEG)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OTz1TMA4)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OTzlChmPEG3)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(SPD)-2Nal-THP-K(NMeAc)—N-3Pya-Sar-CON(Me)2;
cPEG3aCO-Pen(3)—N(N(Me)2)-T-7MeW—K(NMeAc)-Pen(3)-MMoPF-2Nal-THIP-K(NMeAc)—N-3Pya-Sar-CON(Me)2;
cPEG3aCO-Pen(3)—N(N(Me)2)-T-7MeW—K(NMeAc)-Pen(3)-PiperazinequatF-2Nal-THP-K(NMeAc)—N-3Pya-Sar-CON(Me)2;
cPEG3aCO-Pen(3)—N(N(Me)2)-T-7MeW—K(NMeAc)-Pen(3)-mPEG3 TMA4F-2Nal-THP-K(NMeAc)—N-3Pya-Sar-CON(Me)2;
cPEG3aCO-Pen(3)—N(N(Me)2)-T-7MeW—K(NMeAc)-Pen(3)-morfTMA4F-2Nal-THP-K(NMeAc)—N-3Pya-Sar-CON(Me)2;
cPEG3aCO-Pen(3)—N(N(Me)2)-T-7MeW—K(NMeAc)-Pen(3)-DMTASF-2Nal-THP-K(NMeAc)—N-3Pya-Sar-CON(Me)2;
MeCO-Pen(3)—N(N(Me)2)-T-7MeW-APEG2Ser(R*)-Pen(3)-APEG3F-2Nal-THP-K(NMeAc)—N-3Pya-Sar-CON(Me)2;
MeCO-Pen(3)—N(N(Me)2)-T-7MeW-APEG2Ser(S*)-Pen(3)-APEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(BisPEG2a)(S*)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(BisPEG2a)(RS)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(aPEG2a)-2Nal-THP-K(NMeAc)—N-3Pya-Sar-CON(Me)2;
cPEG3aCO-Pen(3)—N(N(Me)2)-T-7MeW—K(NMeAc)-Pen(3)-TBAPEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-MTASF-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(NMe3)-2Nal-THP-K(NMeAc)—N-3Pya-Sar-CON(Me)2;
cPEG3aCO-Pen(3)—N(N(Me)2)-T-7MeW—K(NMeAc)-Pen(3)—YC8CO(NHPEG3a)-2Nal-THP-K(NMeAc)—N-3Pya-Sar-CON(Me)2;
cPEG3aCO-Pen(3)—N(N(Me)2)-T-7MeW-APEG2Ser(S*)-Pen(3)-TMAPF-2Nal-THP-K(NMeAc)—N-3Pya-Sar-CON(Me)2;
cPEG3aCO-Pen(3)—N(N(Me)2)-T-7MeW-APEG2Ser(R*)-Pen(3)-TMAPF-2Nal-THP-K(NMeAc)—N-3Pya-Sar-CON(Me)2;
cPEG3aCO-Pen(3)—N(N(Me)2)-T-7MeW-APEG2Ser-Pen(3)-AEF-2Nal-THP-K(NMeAc)—N-3Pya-Sar-CON(Me)2;
cPEG3aCO-Pen(3)—N(N(Me)2)-T-7MeW-APEG2Ser(R*)-Pen(3)-AEF-2Nal-THP-K(NMeAc)—N-3Pya-Sar-CON(Me)2;
cPEG3aCO-Pen(3)—N(N(Me)2)-T-7MeW—K(Ac)-Pen(3)-APEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-YC8COPip-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(NMeBismPEG3)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(NMe2mPEG3)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(PEG2a)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(NMePEG2a)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(C9OH)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(MePrpa)-2Nal-THP-K(NMeAc)—N-3Pya-Sar-CON(Me)2;
MeCO-APEG2ser-Pen(3)—N(N(Me)2)-T-7MeW—K(NMeAc)-Pen(3)-APEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-NPyEF-2Nal-THP-K(NMeAc)—N-3Pya-Sar-CON(Me)2;
cPEG3aCO-Pen(3)—N(N(Me)2)-T-7MeW—K(NMeAc)-Pen(3)-TMA3F-2Nal-THP-K(NMeAc)—N-3Pya-Sar-CON(Me)2;
cPEG3aCO-Pen(3)—N(N(Me)2)-T-7MeW—K(NMeAc)-Pen(3)-TMA3F-2Nal-THP-K(NMeAc)—N-3Pya-Sar-CON(Me)2;
cPEG3aCO-Pen(3)—N(N(Me)2)-T-7MeW—K(NMeAc)-Pen(3)-mPEG2TMA2F-2Nal-THP-K(NMeAc)—N-3Pya-Sar-CON(Me)2;
cPEG3aCO-Pen(3)—N(N(Me)2)-T-7MeW—K(NMeAc)-Pen(3)-mPEG2TMA4F-2Nal-THP-K(NMeAc)—N-3Pya-Sar-CON(Me)2;
cPEG3aCO-Pen(3)—N(N(Me)2)-T-7MeW—K(NMeAc)-Pen(3)-TMA4F-2Nal-THP-K(NMeAc)—N-3Pya-Sar-CON(Me)2;
mPEG3CO-SP6-Pen(3)—N(N(Me)2)-T-7MeW—K(NMeAc)-Pen(3)-APEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-MPzPEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-MMPEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-ACHMF-2Nal-THP-K(NMeAc)—N-3Pya-Sar-CON(Me)2;
MeCO-Pen(3)—N(N(Me)2)-T-7MeW—K(NMePEG3a)-Pen(3)-APEG3F-2Nal-THP-K(NMeAc)—N-3Pya-Sar-CON(Me)2;
MeCO-k(Me)₃-Pen(3)—N(N(Me)2)-T-7MeW—K(NMeAc)-Pen(3)-APEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-DMMMF-2Nal-THP-K(NMeAc)—N-3Pya-Sar-CON(Me)2;
MeCO-Pen(3)—N(N(Me)2)-T-7MeW-Dab(NMecarn)-Pen(3)-APEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-ACHMF-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF((Ch)cPEG3a)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(AcCh)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(NsCh)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(BisMEP))-2Nal-THP-K(NMeAc)—N-3Pya-Sar-CON(Me)2;
cPEG3aCO-Pen(3)—N(N(Me)2)-T-7MeW—K(NMeAc)-Pen(3)-DMPMF-2Nal-THP-K(NMeAc)—N-3Pya-Sar-CON(Me)2;
cPEG3aCO-Pen(3)—N(N(Me)2)-T-7MeW—K(NMeAc)-Pen(3)-APEG2F-2Nal-THP-K(NMeAc)—N-3Pya-Sar-CON(Me)2;
cPEG3aCO-Pen(3)—N(N(Me)2)-T-7MeW—K(NMeAc)-Pen(3)-MMoEF-2Nal-THP-K(NMeAc)—N-3Pya-Sar-CON(Me)2;
cPEG3aCO-Pen(3)—N(N(Me)2)-T-7MeW—K(NMeAc)-Pen(3)-4DMPzEF-2Nal-THP-K(NMeAc)—N-3Pya-Sar-CON(Me)2;
MeCO-Pen(3)—N(N(Me)2)-T-7MeW—K(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(NMeAc)-Pen(3)-4DMPEF-2Nal-THP-K(NMeAc)—N-3Pya-Sar-CON(Me)2;
MeCO-Pen(3)—N(N(Me)2)-T-7MeW—K(NMeAc)-Pen(3)-APEG3F-2Nal-TTHP-K(NMeAc)—N-3Pya-Sar-CON(Me)2;
cPEG3aCO-Pen(3)—N(N(Me)2)-T-7MeW—K(NMeAc)-Pen(3)-APEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-THP-K(NMeAc)—N-3Pya-Sar-CON(Me)2;
MeCO-Pen(3)—N(N(Me)2)-T-7MeW—K(NMeAc)-Pen(3)-AEF(N(Me)2)-2Nal-THP-K(NMeAc)—N-3Pya-Sar-CON(Me)2;
CF3CO-Pen(3)—N(N(Me)2)-T-7CF3W—K(NMeAc)-Pen(3)-AEF(N(Me)2)-2Nal-THP-K(NMeAc)—N-3Pya-Sar-CON(Me)2;
CF3CO-Pen(3)—N(N(Me)2)-T-7CF3W—K(NMeAc)-Pen(3)-AEF-2Nal-THP-K(NMeAc)—N-3Pya-Sar-CON(Me)2;
CF3CO-Pen(3)—N(N(Me)2)-T-7MeW—K(NMeAc)-Pen(3)-AEF-2Nal-THP-K(NMeAc)—N-3Pya-Sar-CON(Me)2;
CF3CO-Pen(3)—N(N(Me)2)-T-7MeW—K(NMeAc)-Pen(3)-AEF(N(Me)2)-2Nal-THP-K(NMeAc)—N-3Pya-Sar-CON(Me)2;
MeCO-Pen(3)—N(N(Me)2)-T-7MeW—K(NMeAc)-Pen(3)-APEG3F-2Nal-TTHP-K(NMePEG3a)—N-3Pya-Sar-CON(Me)2;
cPEG3aCO-Pen(3)—N(N(Me)2)-T-7MeW—K(NMeAc)-Pen(3)-TMAPF-2Nal-THP-K(PEG2PEG2gEC12)—N-3Pya-Sar-CON(Me)2;
cPEG3aCO-Pen(3)—N(N(Me)2)-T-7MeW—K(PEG2PEG2gEC12)-Pen(3)-TMAPF-2Nal-THP-K(PEG2PEG2gEC12)—N-3Pya-Sar-CON(Me)2;
cPEG3aCO-k(PEG2PEG2gEC12)-Pen(3)—N(N(Me)2)-T-7MeW—K(NMeAc)-Pen(3)-TMAPF-2Nal-THP-K(PEG2PEG2gEC12)—N-3Pya-Sar-CON(Me)2;
C12gEPEG2PEG2CO-hk(Me)₃-Pen(3)—N(N(Me)2)-T-7MeW—K(NMeAc)-Pen(3)-TMAPF-2Nal-THP-K(PEG2PEG2gEC12)—N-3Pya-Sar-CON(Me)2;
C12gEPEG2PEG2CO-hk(Me)₃-Pen(3)—N(N(Me)2)-T-7MeW—K(NMeAc)-Pen(3)-TMAPF-2Nal-K(PEG2PEG2gEC12)—N-3Pya-Sar-CON(Me)2;
cPEG3aCO-Pen(3)—N(N(Me)2)-T-7MeW—K(PEG2PEG2gEC14)-Pen(3)-TMAPF-2Nal-THP-K(PEG2PEG2gEC14)—N-3Pya-Sar-CON(Me)2;
cPEG3aCO-Pen(3)—N(N(Me)2)-T-7MeW—K(NMeAc)-Pen(3)-TMAPF-2Nal-THP-K(PEG2PEG2gEC14)—N-3Pya-Sar-CON(Me)2;
cPEG3aCO-k(PEG2PEG2gEC14)-Pen(3)—N(N(Me)2)-T-7MeW—K(NMeAc)-Pen(3)-TMAPF-2Nal-THP-K(PEG2PEG2gEC14)—N-3Pya-Sar-CON(Me)2;
C14gEPEG2PEG2CO-hk(Me)₃-Pen(3)—N(N(Me)2)-T-7MeW—K(NMeAc)-Pen(3)-TMAPF-2Nal-THP-K(PEG2PEG2gEC14)—N-3Pya-Sar-CON(Me)2;
cPEG3aCO-Pen(3)—N(N(Me)2)-T-7MeW—K(NMeAc)-Pen(3)-APEG3F-2Nal-THP-L-N-3Pya-Sar-CON(Me)2;
5cpaCO-Pen(3)—N(N(Me)2)-T-7MeW-Q(N(Me)2)-Pen(3)-AEF(N(Me)2)-2Nal-Pip(NMe2)-Q(N(Me)2)—N-3Pya-Sar-CON(Me)2;
cPEG3aCO-Pen(3)—N(N(Me)2)-T-7MeW—K(NMeAC)-Pen(3)-TMAPF-2Nal-THP-Q(N(Me)2)—N-3Pya-Sar-CON(Me)2;
cPEG3aCO-Pen(3)—N(N(Me)2)-T-7MeW-Q(N(Me)2)-Pen(3)-AEF(N(Me)2)-2Nal-THP-Q(N(Me)2)—N-3Pya-Sar-CON(Me)2;
cPEG3aCO-Pen(3)—N(N(Me)2)-T-7MeW-Q(N(Me)2)-Pen(3)-AEF-2Nal-THP-Q(N(Me)2)—N-3Pya-Sar-CON(Me)2;
MeCO-Pen(3)—N(N(Me)2)-T-7MeW-Q(N(Me)2)-Pen(3)-AEF(N(Me)2)-2Nal-THP-Q(N(Me)2)—N-3Pya-Sar-CON(Me)2;
5cpaCO-Pen(3)—N(N(Me)2)-T-7MeW-Q(N(Me)2)-Pen(3)-TMAPF-2Nal-THP-Q(N(Me)2)—N-3Pya-Sar-CON(Me)2;
CF3CO-k(5cpa)-Pen(3)—N(N(Me)2)-T-7MeW-Q(N(Me)2)-Pen(3)-AEF(N(Me)2)-2Nal-THP-Q(N(Me)2)—N-3Pya-Sar-CON(Me)2;
5cpaCO-Pen(3)—N(N(Me)2)-T-7MeW-Q(N(Me)2)-Pen(3)-AEF(N(Me)2)-2Nal-THP-Q(N(Me)2)—N-3Pya-Sar-CON(Me)2;
cPEG3aCO-Pen(3)—N(N(Me)2)-T-7MeW—K(NMeAc)-Pen(3)-APEG3F-2Nal-THP—W—N-3Pya-Sar-CON(Me)2;
cPEG3aCO-Pen(3)—N(N(Me)2)-T-7MeW—K(NMeAc)-Pen(3)-AEF(BisMEPa)-2Nal-THP-K(NMeAc)—N-3Pya-Sar-CON(Me)2;
cPEG3aCO-Pen(3)—N(N(Me)2)-T-7MeW-Dab(NMeAc)-Pen(3)-AEF-2Nal-THP-Dab(NMeAc)—N-3Pya-Sar-CONH(PEG3a);
cPEG3aCO-Pen(3)—N(N(Me)2)-T-7MeW—K(NMeAC)-Pen(3)-TMAPF-2Nal-THP-K(NMeAC)—N-3Pya-NMeK(PEG2PEG2C12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG2C14)—CONH2;
C12gEPEG2PEG2CO-Pen(3)—N(N(Me)2)-T-7MeW—K(Ac)-Pen(3)-TMAPF-2Nal-THP-K(NMeAc)—N-3Pya-NMeK(PEG2PEG2gEC12)—CONH2;
cPEG3aCO-Pen(3)—N(N(Me)2)-T-7MeW—K(PEG2PEG2gEC12)-Pen(3)-TMAPF-2Nal-THP-K(NMeAc)—N-3Pya-NMeK(PEG2PEG2gEC12)—CONH2;
cPEG3aCO-k(PEG2PEG2gEC12)-Pen(3)—N(N(Me)2)-T-7MeW—K(Ac)-Pen(3)-TMAPF-2Nal-THP-K(NMeAc)—N-3Pya-NMeK(PEG2PEG2gEC12)—CONH2;
cPEG3aCO-Pen(3)—N(N(Me)2)-T-7MeW—K(NMeAC)-Pen(3)-TMAPF-2Nal-THP-K(NMeAC)—N-3Pya-NMeK(PEG2PEG2gEC12)—CONH2;
cPEG3aCO-Pen(3)—N(N(Me)2)-T-7MeW—K(Ac)-Pen(3)-TMAPF-2Nal-THP-K(NMeAC)—N-3Pya-NMeK(PEG2PEG2gEC12)—CONH2;
cPEG3aCO-Pen(3)—N(N(Me)2)-T-7MeW—K(Ac)-Pen(3)-TMAPF-2Nal-THP-K(PEG2PEG2gEC12)—N-3Pya-NMeK(PEG2PEG2gEC12)—CONH2;
cPEG3aCO-Pen(3)—N(N(Me)2)-T-7MeW—K(PEG2PEG2gEC14)-Pen(3)-TMAPF-2Nal-THP-K(NMeAc)—N-3Pya-NMeK(PEG2PEG2gEC14)—CONH2;
C14gEPEG2PEG2CO-Pen(3)—N(N(Me)2)-T-7MeW—K(Ac)-Pen(3)-TMAPF-2Nal-THP-K(NMeAc)—N-3Pya-NMeK(PEG2PEG2gEC14)—CONH2;
cPEG3aCO-k(PEG2PEG2gEC14)-Pen(3)—N(N(Me)2)-T-7MeW—K(Ac)-Pen(3)-TMAPF-2Nal-THP-K(NMeAc)—N-3Pya-NMeK(PEG2PEG2gEC14)—CONH2;
cPEG3aCO-Pen(3)—N(N(Me)2)-T-7MeW—K(NMeAC)-Pen(3)-TMAPF-2Nal-THP-K(NMeAC)—N-3Pya-NMeK(PEG2PEG2gEC14)—CONH2;
cPEG3aCO-Pen(3)—N(N(Me)2)-T-7MeW—K(Ac)-Pen(3)-TMAPF-2Nal-THP-K(PEG2PEG2gEC14)—N-3Pya-NMeK(PEG2PEG2gEC14)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG2K(PEG2PEG2gEC12)₂)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG2K(PEG2PEG2PEG2PEG2gEC12)₂)—CONH2;
cPEG3aCO-Pen(3)—N(N(Me)2)-T-7MeW—K(Ac)-Pen(3)-TMAPF-2Nal-THP-K(PEG2PEG2PEG2gEC12))—N-3Pya-NMeK(PEG2PEG2PEG2gEC12)—CONH2;
cPEG3aCO-k(PEG2PEG2PEG2PEG2gEC12)-Pen(3)—N(N(Me)2)-T-7MeW—K(Ac)-Pen(3)-TMAPF-2Nal-THP-K(NMeAc)—N-3Pya-NMeK(PEG2PEG2PEG2PEG2gEC12)—CONH2;
cPEG3aCO-k(PEG2PEG6gEC12)-Pen(3)—N(N(Me)2)-T-7MeW—K(Ac)-Pen(3)-TMAPF-2Nal-THP-K(NMeAc)—N-3Pya-NMeK(PEG2PEG6gEC12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG6gEC12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG6gEC14)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(SP6PEG2PEG2C12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(SP6PEG2PEG2gEC12)—CONH2;
MeCO-Pen(3)—N(N(Me)2)-T-7MeW—K(Ac)-Pen(3)-AEF-2Nal-THP—K(Ac)—N-3Pya-Sar-CONH2;
cPEG3aCO-Pen(3)—N(N(Me)2)-T-7MeW—K(NMeAc)-Pen(3)-AEF-2Nal-THP—K(Ac)—N-3Pya-Sar-CONH2;
mPEG3CO-SP6-Pen(3)—N(N(Me)2)-T-7MeW—K(NMeAc)-Pen(3)-AEF-2Nal-THP—K(Ac)—N-3Pya-Sar-CONH2;
CF3CO-Pen(3)—N(N(Me)2)-T-7MeW—K(Ac)-Pen(3)-AEF-2Nal-THP—K(Ac)—N-3Pya-Sar-CONH2;
cPEG3aCO-Pen(3)—N(N(Me)2)-T-7MeW—K(NMeAc)-Pen(3)-APEG3F-2Nal-THP-K(NMeAc)—N-3Pya-Sar-CONH2;
cPEG3aCO-Pen(3)—N(N(Me)2)-T-7MeW-Dab(NMeAc)-Pen(3)-TMAPF-2Nal-THP-K(NMeAc)—N-3Pya-Sar-CONH2;
cPEG3aCO-Pen(3)—N(N(Me)2)-T-7MeW—K(NMeAc)-Pen(3)-TMAPF-2Nal-THP-K(NMeAc)—N-3Pya-Sar-CONH2;
MeCO-Pen(3)—N(N(Me)2)-T-7MeW—K(NMeAc)-Pen(3)-AEF-2Nal-THP-K(NMeAc)—N-3Pya-Sar-CONH2;
cPEG3aCO-Pen(3)—N(N(Me)2)-T-7MeW-Dab(NMeAc)-Pen(3)-AEF-2Nal-THP-K(NMeAc)—N-3Pya-Sar-CONH2;
cPEG3aCO-Pen(3)—N(N(Me)2)-T-7MeW—K(NMeAc)-Pen(3)-AEF-2Nal-THP-K(NMeAc)—N-3Pya-Sar-CONH2;
cPEG3aCO-Pen(3)—N(N(Me)2)-T-7MeW—K(NMeAc)-Pen(3)-AEF-2Nal-THP-Q(N(Me)2)—N-3Pya-Sar-CONH2;
cPEG3aCO-Pen(3)—N(N(Me)2)-T-7MeW-Q(N(Me)2)-Pen(3)-AEF-2Nal-THP-Q(N(Me)2)—N-3Pya-Sar-CONH2;
mPEG3CO-SP6-Pen(3)—N(N(Me)2)-T-7MeW-Q(N(Me)2)-Pen(3)-AEF-2Nal-THP-Q(N(Me)2)—N-3Pya-Sar-CONH2;
CF3CO-Pen(3)—N(N(Me)2)-T-7MeW—K(NMeAc)-Pen(3)-AEF(N(Me)2)-2Nal-THP-Q(N(Me)2)—N-3Pya-Sar-CONH2;
mPEG3CO-SP6-Pen(3)—N(N(Me)2)-T-7MeW—K(NMeAc)-Pen(3)-AEF-2Nal-THP-QNMe2-N-3Pya-Sar-CONH2;
cPEG3aCO-Pen(3)—N(NMe2)-T-7MeW—K(NMeAc)-Pen(3)-AEF(NHCh)-2Nal-THP-K(NMeAc)—N-3Pya-Sar-CON(Me)2;
   or a pharmaceutically acceptable salt thereof.

In another aspect of the invention:

10. The compound of aspect 3 wherein:
R⁹ is —NHCH₃, —N(CH₃)₂, —NH(CH₂CH₂O)₂CH₃, —N(CH₃)(CH₂CH₂O)₂CH₃, —NH(CH₂CH₂O)_{y}R^{s},

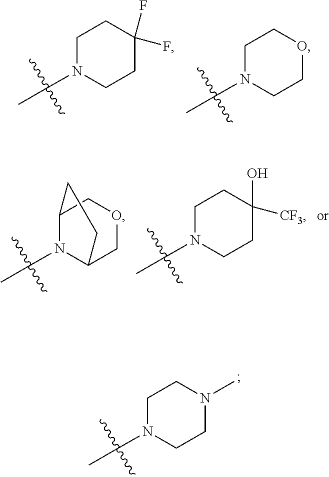

wherein:
   y is 2, 4, or 6;
   R^{s} is —CH₃, or

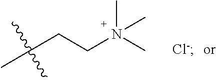

a pharmaceutically acceptable salt thereof.

In another aspect of the invention:

11. A compound of Formula (IV):

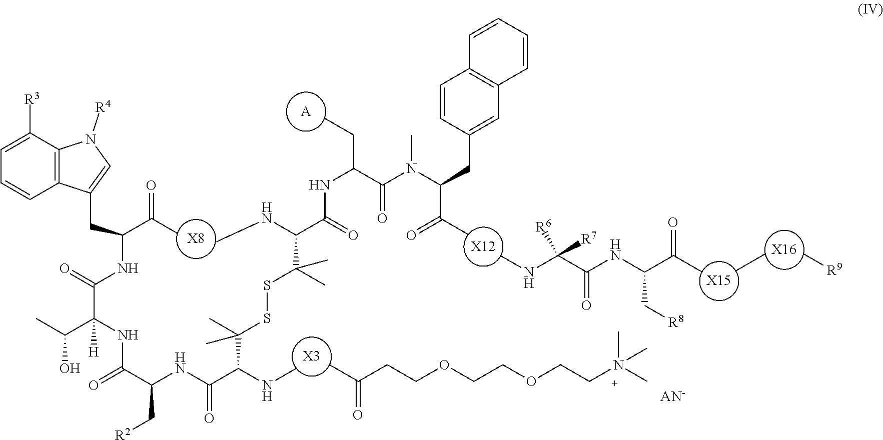

wherein:
A is

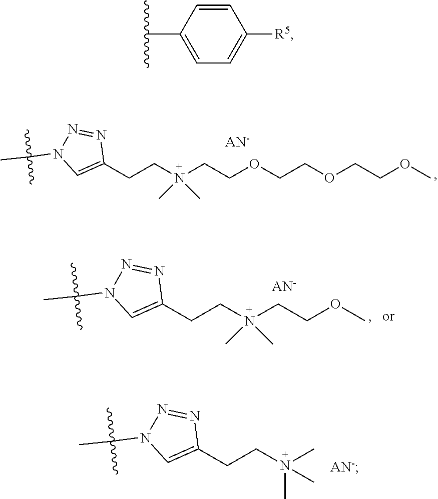

wherein:
   R⁵ is —OCHF₂, —O(CH₂)₉CO₂H,

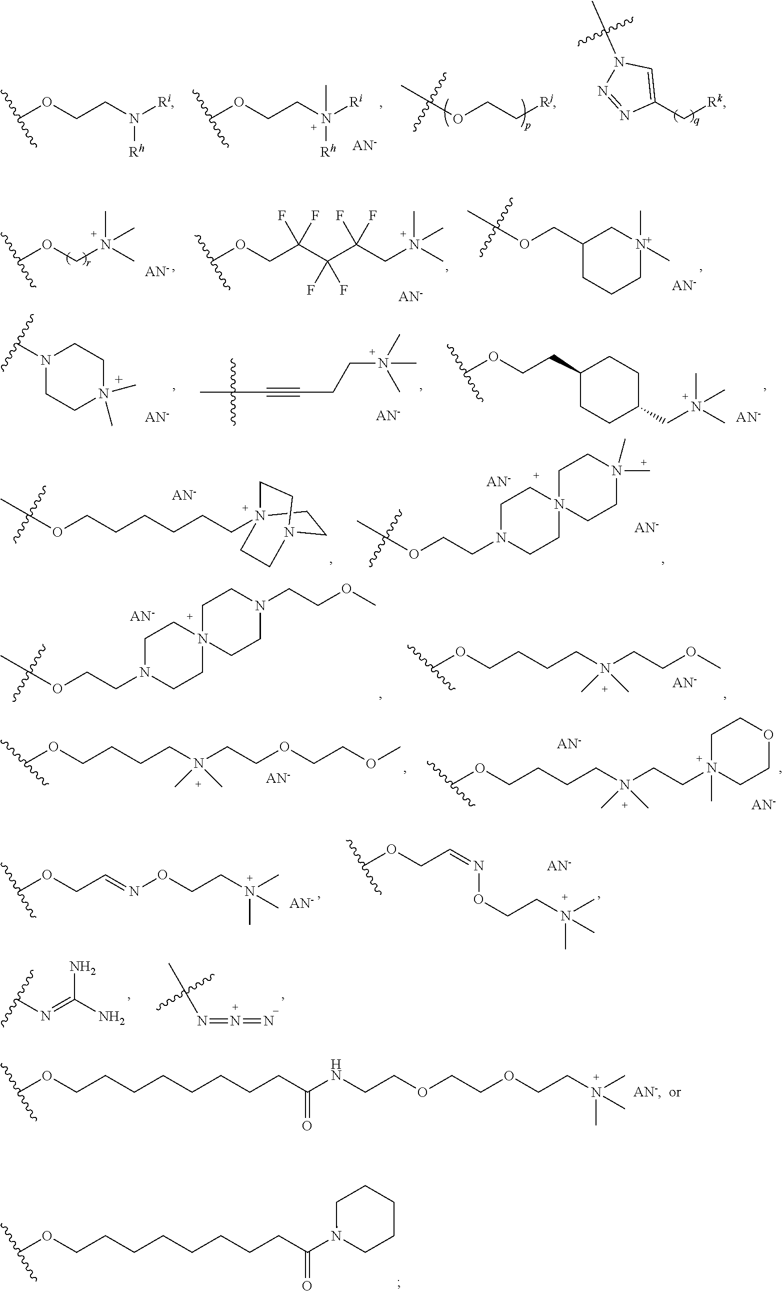

wherein:
   R^{h} is H, —CH₃, —C(O)CH₃, —C(NH)NH₂, —(CH₂)₃O(CH₂)₂OCH₃, —CH₂CH₂OCH₃, (CH₂CH₂O)₃CH₃, —(CH₂CH₂O)₆CH₃,

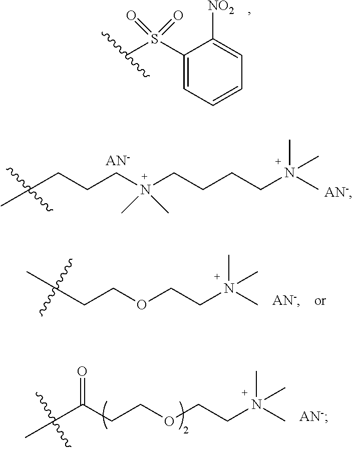

Rⁱ is H, —CH₃, —(CH₂)₃O(CH₂)₂OCH₃,

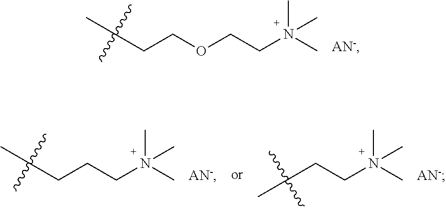

R^{j} is

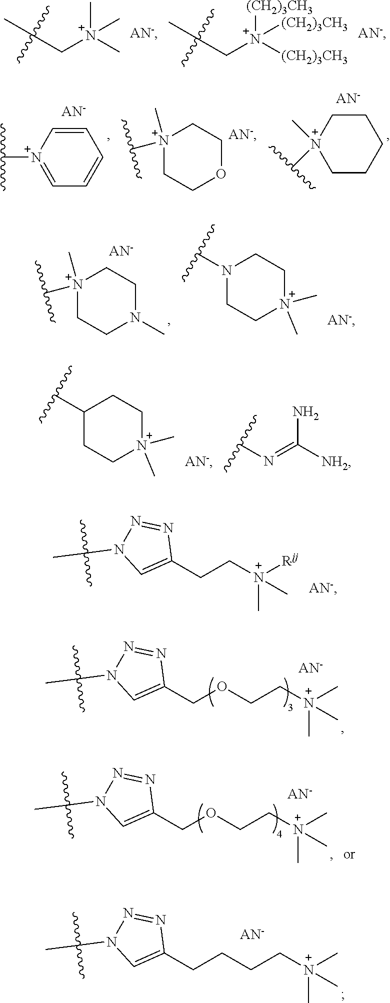

q is 1, 2, 3, 4, or 5;
R^{k} is —OCH₃,

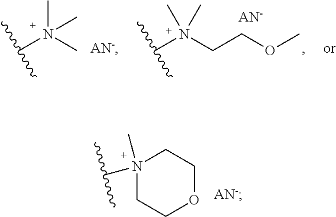

r is 3, 4, 5, 6, or 8;
wherein:
   R^{jj} is H, —(CH₂)₇CH₃, —(CH₂)₁₅CH₃, —(CH₂)₂OCH₃, or —(CH₂CH₂O)₃CH₃;
X3 is a direct bond, (D) arginine, (D) lysine substituted with R^{b},

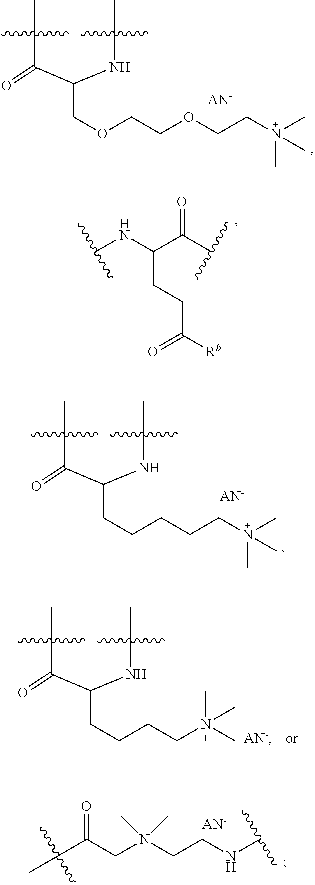

wherein:
   R^{b} is

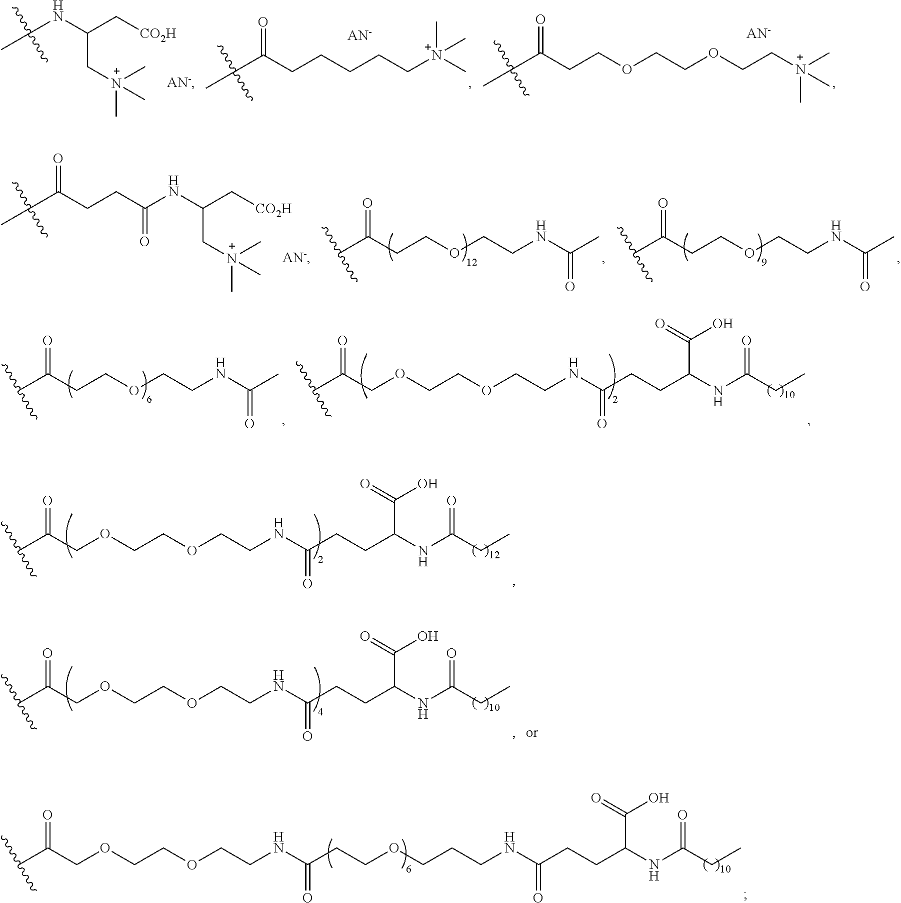

R² is H, —CH(CH₃)₂,

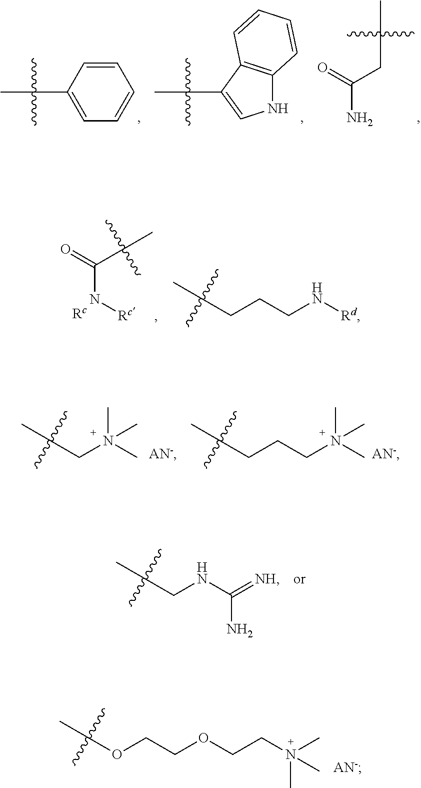

wherein
   R^{c} is H, or —CH₃;
   R^{c}′ is H, or —CH₃;
R^{d} is

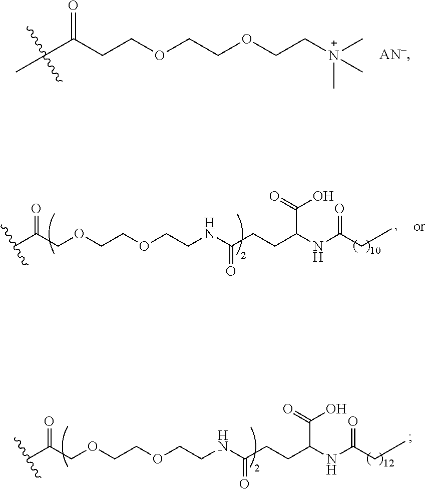

R³ is H, —CH₃, —CF₃, or

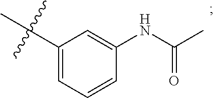 [SMILES: *c1cccc(NC(C)=O)c1]

R⁴ is H, or —CH₃;
X8

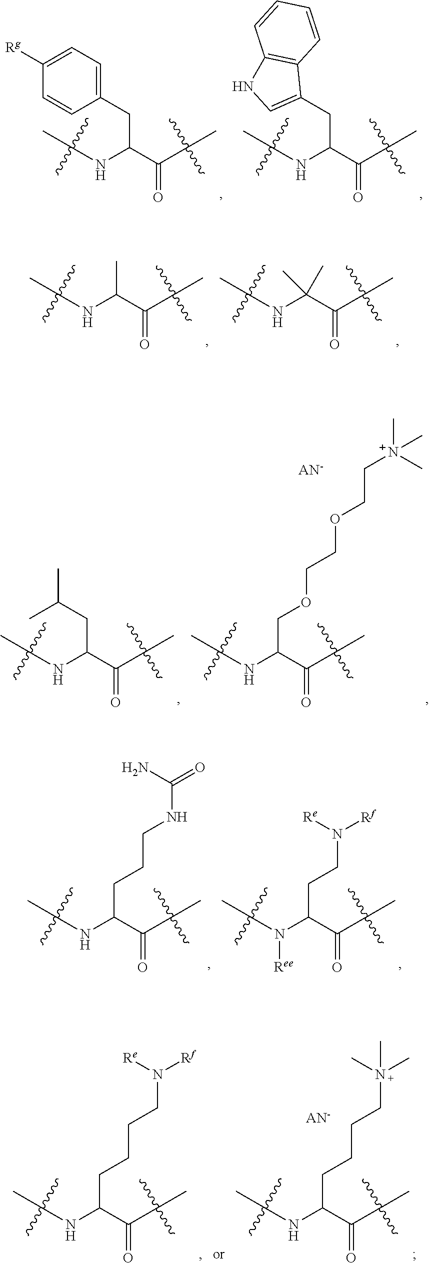

wherein:
   R^{e} is H, or —CH₃;
   R^{ee} is H;
   R^{f} is H, —CH₃, —C(CH₃)₃, —C(O)CH₃, —C(O)(CH₂CH₂O)₆CH₃, —(CH₂CH₂O)₆CH₃,

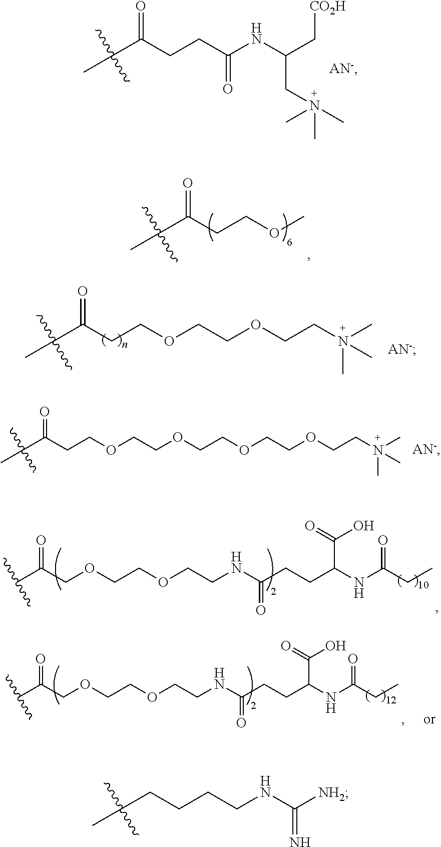

R^{g} is H, —OH, —C(O)NH₂, or —CH₂NHC(O)CH₃;
n is 0, or 1
X12 is absent,

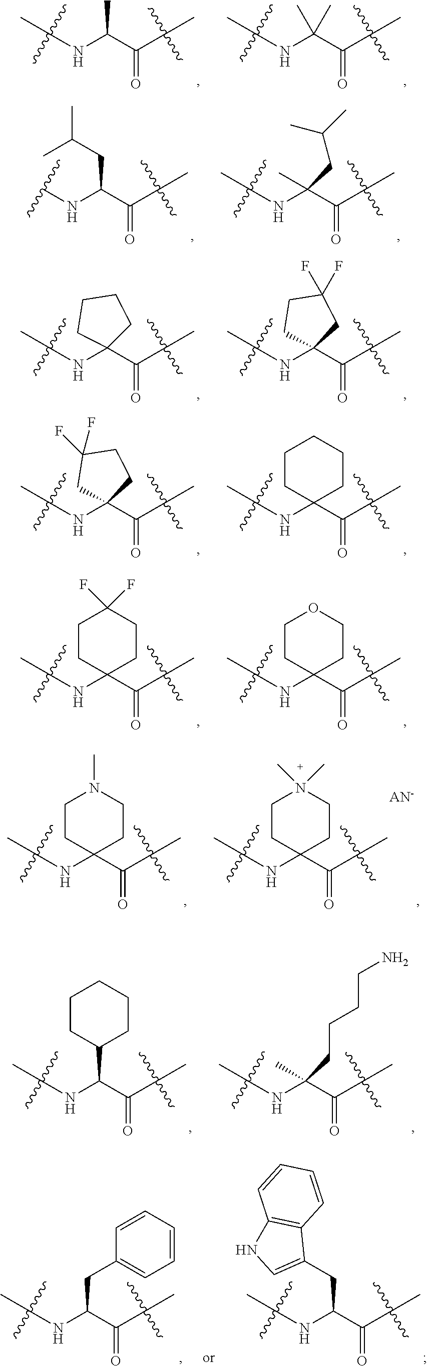

R⁶ is —CH₃, —CH₂CH₂C(O)R^{m}, —(CH₂)ₛNRⁿR^{o},

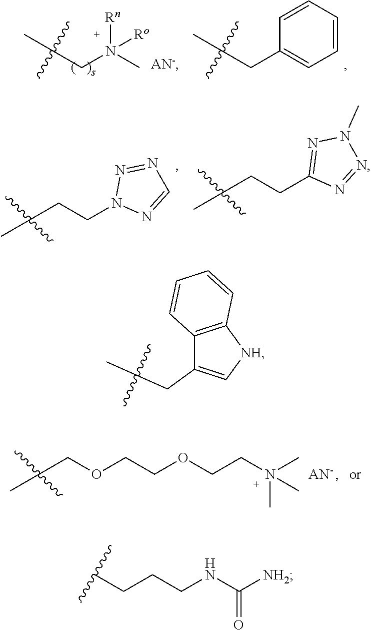

R⁷ is H, or —CH₃;
wherein:
   R^{m} is —OH, or

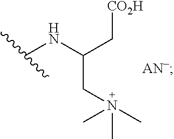

Rⁿ is H, —CH₃, —CH₂CH(CH₃)₂, —C(O)CH₃, —C(O)(CH₂CH₂O)₆CH₃, —(CH₂CH₂O)₆CH₃, —C(O)(CH₂CH₂O)ₜCH₂CH₂R^{p},

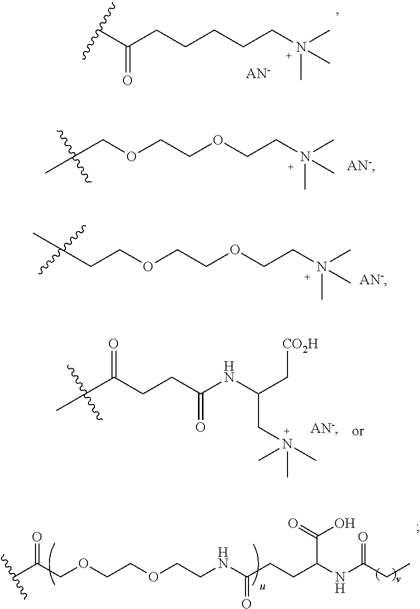

R^{o} is H, —CH₃, or —C(O)CH₃;
   wherein:
      R^{p} is NHC(O)CH₃, or

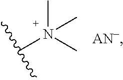

s is 1, 2, or 4;
t is 2, 6, 9, or 12;
u is 2 or 3;
v is 10 or 12;
R⁸ is —CH₂CH₂CH₂NHC(O)CH₃, —C(O)NH₂, or —C(O)NHCH₃;
X15 is absent,

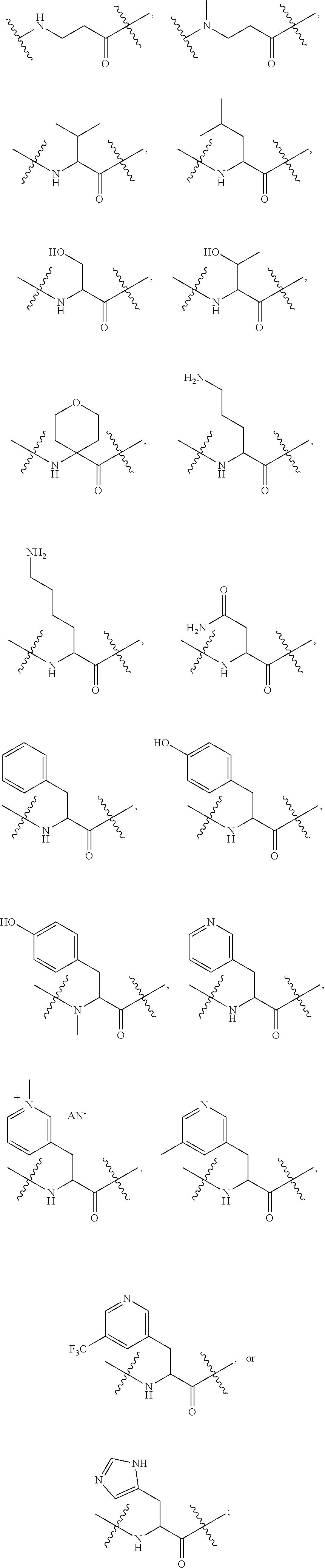

X16 is absent,

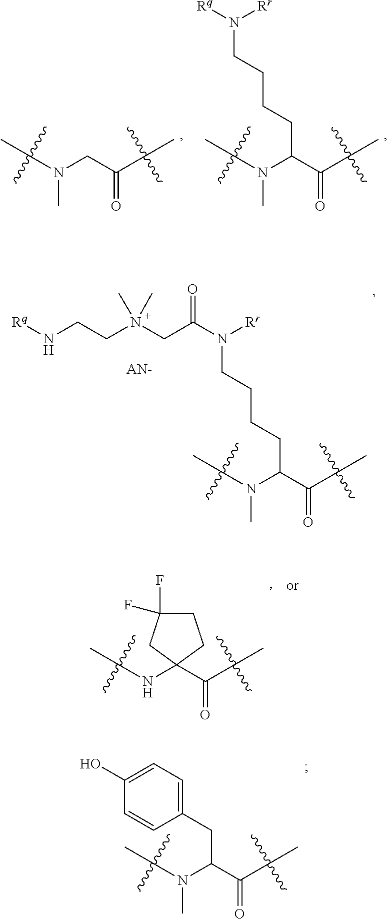

wherein:
   R^{q} is

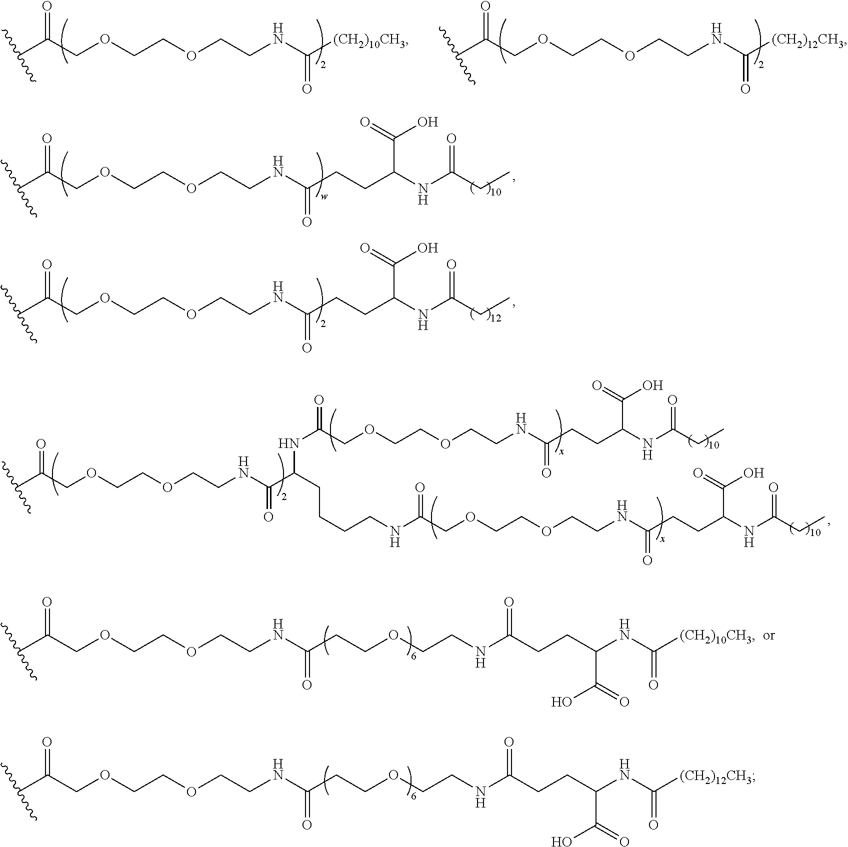

R^{r} is H;
w is 2 4, or 5;
x is 2 or 4; and
R⁹ is —NH₂, —NHCH₃, —N(CH₃)₂, —NH(CH₂CH₂O)₂CH₃, —N(CH₃)(CH₂CH₂O)₂CH₃, —NH(CH₂CH₂O)_{y}R^{s},

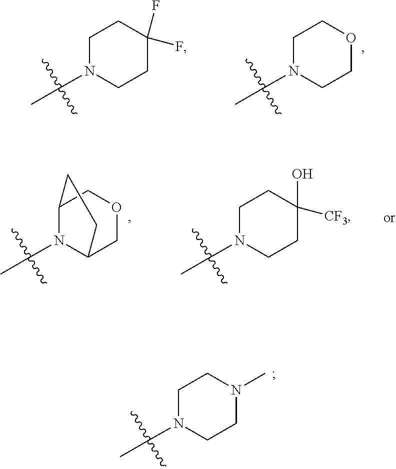

wherein:
   y is 2, 4, or 6;
R^{s} is —CH₃, or

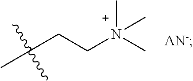 [SMILES: *CC[N+](C)(C)C]

wherein:
   AN⁻as above defined for A, R, X3, X12, R1, R2, R5, R6, R8, R15, R16 and R^{a}, R^{b}, R^{d}, R^{f}, R^{h}, Rⁱ, R^{j}, R^{k}, R^{m}, R″, R^{o}, R^{s} is acetate, adipate, benzoate, benzenesulfonate, citrate, decanoate, chloride, lactate, maleate, methanesulfonate, oxalate, pivalate, propionate, succinate, sulfate, tartrate, or trifluoroacetate; or a pharmaceutically acceptable salt thereof.

In another aspect of the invention:

12. The compound of aspect 11 wherein:
AN⁻is chloride;
or a pharmaceutically acceptable salt thereof.

In another aspect of the invention:

13. The compound of aspect 11 selected from
cPEG3aCO-Pen(3)—N(N(Me)2)-T-7MeW—K(NMeAc)-Pen(3)-4DMPEF-2Nal-THP-K(NMeAc)—N-3Pya-Sar-CON(Me)2;
cPEG3aCO-Pen(3)—N(N(Me)2)-T-7MeW—K(NMeAc)-Pen(3)-4DMPzEF-2Nal-THP-K(NMeAc)—N-3Pya-Sar-CON(Me)2;
cPEG3aCO-Pen(3)—N(N(Me)2)-T-7MeW—K(NMeAc)-Pen(3)-ACHMF-2Nal-THP-K(NMeAc)—N-3Pya-Sar-CON(Me)2;
cPEG3aCO-Pen(3)—N(N(Me)2)-T-7MeW—K(NMeAc)-Pen(3)-ACHMF-2Nal-THP-K(NMeAc)—N-3Pya-Sar-CON(Me)2;
cPEG3aCO-Pen(3)—N-T-7MeW—K(Ac)-Pen(3)-AEF-2Nal-THP-E-N-3Pya-Sar-CONH2;
cPEG3aCO-Pen(3)—N-T-7MeW—K(Ac)-Pen(3)-AEF-2Nal-THP—K(Ac)—N-3Pya-Sar-CONH2;
cPEG3aCO-Pen(3)—N-T-7MeW-Q(N(Me)2)-Pen(3)-AEF-2Nal-THP-Q(N(Me)2)—N-3Pya-Sar-CONH2;
cPEG3aCO-Pen(3)—N-T-7MeW-Q(N(Me)2)-Pen(3)-AEF-2Nal-THP-Q(N(Me)2)—N-3Pya-Sar-CON(Me)2;
cPEG3aCO-Pen(3)—N-T-7MeW—K(NMeAc)-Pen(3)-AEF-2Nal-THP-E-N-3Pya-Sar-CONH2;
cPEG3aCO-Pen(3)—N-T-7MeW—K(NMeAc)-Pen(3)-AEF-2Nal-THP—K(Ac)—N-3Pya-Sar-CONH2;
cPEG3aCO-Pen(3)—N-T-7MeW—K(NMeAc)-Pen(3)-AEF-2Nal-THP-K(NMeAc)—N-3Pya-Sar-CONH2;
cPEG3aCO-Pen(3)—N-T-7MeW-Q(N(Me)2)-Pen(3)-AEF-2Nal-THP-K(NMeAc)—N-3Pya-Sar-CONH2;
cPEG3aCO-Pen(3)—N-T-7MeW—K(NMeAc)-Pen(3)-AEF-2Nal-THP-Dab(NMeAc)—N-3Pya-Sar-CONH2;
cPEG3aCO-Pen(3)—N-T-7MeW-Q(N(Me)2)-Pen(3)-AEF-2Nal-THP-Dab(NMeAc)—N-3Pya-Sar-CONH2;
cPEG3aCO-Pen(3)—N-T-7(3NAcPh)W—K(Ac)-Pen(3)-AEF-2Nal-THP-E-N-3Pya-Sar-CONH2;
cPEG3aCO-Pen(3)—N-T-7(3NAcPh)W—K(Ac)-Pen(3)-AEF-2Nal-THP—K(Ac)—N-3Pya-Sar-CONH2;
cPEG3aCO-r-Pen(3)—N-T-7MeW—K(Ac)-Pen(3)-AEF-2Nal-THP-E-N-5MePyridinAla-Sar-CONH2;
cPEG3aCO-Pen(3)—N(N(Me)2)-T-7MeW-Q(N(Me)2)-Pen(3)-AEF-2Nal-THP-Q(N(Me)2)—N-3Pya-Sar-CON(Me)2;
cPEG3aCO-Pen(3)—N(N(Me)2)-T-7MeW-AIB-Pen(3)-AEF-2Nal-THP-AIB-N-3Pya-Sar-CON(Me)2;
cPEG3aCO-Pen(3)—N(N(Me)2)-T-7MeW-Q(N(Me)2)-Pen(3)-AEF-2Nal-THP-Q(N(Me)2)—N-3Pya-Sar-CONH2;
cPEG3aCO-Pen(3)—N(N(Me)2)-T-7MeW—K(NMeAc)-Pen(3)-AEF-2Nal-THP—K(Ac)—N-3Pya-Sar-CONH2;
cPEG3aCO-Pen(3)—N(N(Me)2)-T-7MeW—K(NMeAc)-Pen(3)-AEF-2Nal-THP-Q(N(Me)2)—N-3Pya-Sar-CONH2;
cPEG3aCO-Pen(3)—N(N(Me)2)-T-7MeW-Q(N(Me)2)-Pen(3)-AEF-2Nal-THP-Dab(NMeAc)—N-3Pya-Sar-CON(Me)2;
cPEG3aCO-Pen(3)—N(N(Me)2)-T-7MeW-Dab(NMeAc)-Pen(3)-AEF-2Nal-THP-Dab(NMeAc)—N-3Pya-Sar-CON(Me)2;
cPEG3aCO-Pen(3)—N(N(Me)2)-T-7MeW—K(NMeAc)-Pen(3)-AEF-2Nal-THP-K(NMeAc)—N-3Pya-Sar-CONH2;
cPEG3aCO-Pen(3)—N(N(Me)2)-T-7MeW-Dab(NMeAc)-Pen(3)-AEF-2Nal-THP-K(NMeAc)—N-3Pya-Sar-CONH2;
cPEG3aCO-Pen(3)—N(N(Me)2)-T-7MeW-Dab(NMeAc)-Pen(3)-AEF-2Nal-THP-E-N-3Pya-Sar-CONH2;
cPEG3aCO-Pen(3)—N(N(Me)2)-T-7MeW—K(NMeAc)-Pen(3)-AEF-2Nal-THP-Dab(NMeAc)—N-3Pya-Sar-CON(Me)2;
cPEG3aCO-Pen(3)—N(N(Me)2)-T-7MeW—K(NMeAc)-Pen(3)-AEF-2Nal-diFCpx-Dab(NMeAc)—N-3Pya-Sar-CON(Me)2;
cPEG3aCO-Pen(3)—N(N(Me)2)-T-7MeW—K(NMeAc)-Pen(3)-AEF-2Nal-diFCpx-Dab(NMeAc)—N-3Pya-Sar-CON(Me)2;
cPEG3aCO-Pen(3)—N(N(Me)2)-T-7MeW-Dab(NMeAc)-Pen(3)-AEF-2Nal-THP-Dab(NMeAc)—N-3Pya-Sar-CONH(PEG3a);
cPEG3aCO-Pen(3)—N(N(Me)2)-T-7MeW-Dab(NMecPEG3a)-Pen(3)-AEF-2Nal-THP-Dab(NMeAc)—N-3Pya-Sar-CON(Me)2;
cPEG3aCO-Pen(3)—N(N(Me)2)-T-7MeW-APEG2Ser-Pen(3)-AEF-2Nal-THP-K(NMeAc)—N-3Pya-Sar-CON(Me)2;
cPEG3aCO-Pen(3)—N(N(Me)2)-T-7MeW-APEG2Ser(R*)-Pen(3)-AEF-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF((Ch)cPEG3a)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(AcCh)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(aPEG2a)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(BisMEP))-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(BisMEPa)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(BisPEG2a)(RS)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(BisPEG2a)(S*)-2Nal-THP-K(NMeAc)—N-3Pya-Sar-CON(Me)2;
cPEG3aCO-Pen(3)—N-T-7MeW—K(Ac)-Pen(3)-AEF(G)-2Nal-THP—K(Ac)—N-3Pya-Sar-CONH2;
cPEG3aCO-Pen(3)—N(N(Me)2)-T-7MeW—K(NMeAc)-Pen(3)-AEF(MePrpa)-2Nal-THP-K(NMeAc)—N-3Pya-Sar-CON(Me)2;
cPEG3aCO-Pen(3)—N(N(Me)2)-T-7MeW-Q(N(Me)2)-Pen(3)-AEF(N(Me)2)-2Nal-THP-Q(N(Me)2)—N-3Pya-Sar-CON(Me)2;
cPEG3aCO-Pen(3)—N(N(Me)2)-T-7MeW—K(NMeAc)-Pen(3)-AEF(N(Me)2)-2Nal-THP-K(NMeAc)—N-3Pya-Sar-CON(Me)2;
cPEG3aCO-Pen(3)—N(NMe2)-T-7MeW—K(NMeAc)-Pen(3)-AEF(NHCh)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(NMe2mPEG3)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(NMe3)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(NMeBismPEG3)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(NMePEG2a)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(NsCh)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(PEG2a)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(SPD)-2Nal-THP-K(NMeAc)—N-3Pya-Sar-CON(Me)2;
cPEG3aCO-Pen(3)—N(N(Me)2)-T-7MeW—K(NMeAc)-Pen(3)-APEG2F-2Nal-THP-K(NMeAc)—N-3Pya-Sar-CON(Me)2;
cPEG3aCO-Pen(3)-A-T-7MeW-A-Pen(3)-APEG3F-2Nal-A-K(NMeAc)—N-3Pya-Sar-CON(Me)2;
cPEG3aCO-Pen(3)-APEG2Ser(S*)-T-7MeW—K(NMeAc)-Pen(3)-APEG3F-2Nal-THP-K(NMeAc)—N-3Pya-Sar-CON(Me)2;
cPEG3aCO-Pen(3)-Dab(Me)₃-T-7MeW—K(NMeAc)-Pen(3)-APEG3F-2Nal-THP-K(NMeAc)—N-3Pya-Sar-CON(Me)2;
cPEG3aCO-Pen(3)—F-T-7MeW—F-Pen(3)-APEG3F-2Nal-F—K(NMeAc)—N-3Pya-Sar-CON(Me)2;
cPEG3aCO-Pen(3)-Gab-T-7MeW—K(NMeAc)-Pen(3)-APEG3F-2Nal-THP-K(NMeAc)—N-3Pya-Sar-CON(Me)2;
cPEG3aCO-Pen(3)-L-T-7MeW-L-Pen(3)-APEG3F-2Nal-L-K(NMeAc)—N-3Pya-Sar-CON(Me)2;
cPEG3aCO-Pen(3)—N-T-7MeW—K(Ac)-Pen(3)-APEG3F-2Nal-THP—K(Ac)—N-3Pya-Sar-CONH2;
cPEG3aCO-Pen(3)—N-T-7MeW—K(NMeAc)-Pen(3)-APEG3F-2Nal-THP-K(NMeAc)—N-3Pya-Sar-CONH2;
cPEG3aCO-Pen(3)—N-T-7MeW—K(NMeAc)-Pen(3)-APEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-APEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-APEG3F-2Nal-THP-E-N-3Pya-Sar-CON(Me)2;
cPEG3aCO-Pen(3)—N(N(Me)2)-T-7MeW—K(NMeAc)-Pen(3)-APEG3F-2Nal-THP-Dab(NMeAc)—N-3Pya-Sar-CON(Me)2;
cPEG3aCO-Pen(3)—N(N(Me)2)-T-7MeW—K(NMeAc)-Pen(3)-APEG3F-2Nal-THP-Dab(NMeAc)—N-3Pya-Sar-CON(Me)2;
cPEG3aCO-Pen(3)—N(N(Me)2)-T-7MeW—K(NMeAc)-Pen(3)-APEG3F-2Nal-THP—K(Ac)—N-3Pya-Sar-CON(Me)2;
cPEG3aCO-Pen(3)—N(N(Me)2)-T-7MeW—K(Ac)-Pen(3)-APEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-APEG3F-2Nal-THP-K(NMeAc)—N-3Pya-Sar-CONH2;
cPEG3aCO-Pen(3)—N(N(Me)2)-T-7MeW—K(NMeAc)-Pen(3)-APEG3F-2Nal-THP-Dap(NMeAc)—N-3Pya-Sar-CON(Me)2;
cPEG3aCO-Pen(3)—N(N(Me)2)-T-7MeW—K(NMeAc)-Pen(3)-APEG3F-2Nal-THP-A-N-3Pya-Sar-CON(Me)2;
cPEG3aCO-Pen(3)—N(N(Me)2)-T-7MeW—K(NMeAc)-Pen(3)-APEG3F-2Nal-THP-L-N-3Pya-Sar-CON(Me)2;
cPEG3aCO-Pen(3)—N(N(Me)2)-T-7MeW—K(NMeAc)-Pen(3)-APEG3F-2Nal-THP—F—N-3Pya-Sar-CON(Me)2;
cPEG3aCO-Pen(3)—N(N(Me)2)-T-7MeW—K(NMeAc)-Pen(3)-APEG3F-2Nal-THP—W—N-3Pya-Sar-CON(Me)2;
cPEG3aCO-Pen(3)—W-T-7MeW—W-Pen(3)-APEG3F-2Nal-W—K(NMeAc)—N-3Pya-Sar-CON(Me)2;
cPEG3aCO-Pen(3)—N(N(Me)2)-T-7MeW—K(NMeAc)-Pen(3)-dFPPEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-Diazabiclyclooctane6F-2Nal-THP-K(NMeAc)—N-3Pya-Sar-CON(Me)2;
cPEG3aCO-Pen(3)—N(N(Me)2)-T-7MeW—K(NMeAc)-Pen(3)-DMMMF-2Nal-THP-K(NMeAc)—N-3Pya-Sar-CON(Me)2;
cPEG3aCO-Pen(3)—N(N(Me)2)-T-7MeW—K(NMeAc)-Pen(3)-DMPMF-2Nal-THP-K(NMeAc)—N-3Pya-Sar-CON(Me)2;
cPEG3aCO-Pen(3)—N(N(Me)2)-T-7MeW—K(NMeAc)-Pen(3)-DMTASF-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4G)-2Nal-THP-K(NMeAc)—N-3Pya-Sar-CON(Me)2;
cPEG3aCO-Pen(3)—N-T-7MeW—K(Ac)-Pen(3)—F(4N3)-2Nal-THP—K(Ac)—N-3Pya-Sar-CONH2;
cPEG3aCO-Pen(3)—N(N(Me)2)-T-7MeW—K(NMeAc)-Pen(3)—F(4N3)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4Tz1DMA4mPEG)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4Tz1MME)-2Nal-THP-K(NMeAc)—N-3Pya-Sar-CON(Me)2;
cPEG3aCO-Pen(3)—N-T-7MeW—K(Ac)-Pen(3)—F(4Tz1MMo3)-2Nal-THP—K(Ac)—N-3Pya-Sar-CONH2;
cPEG3aCO-Pen(3)—N(N(Me)2)-T-7MeW—K(NMeAc)-Pen(3)—F(4Tz1MMo4)-2Nal-THP-K(NMeAc)—N-3Pya-Sar-CON(Me)2;
cPEG3aCO-Pen(3)—N-T-7MeW—K(Ac)-Pen(3)—F(4Tz1TMA1)-2Nal-THP—K(Ac)—N-3Pya-Sar-CONH2;
cPEG3aCO-Pen(3)—N(N(Me)2)-T-7MeW—K(NMeAc)-Pen(3)—F(4Tz1TMA1)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4Tz1TMA2)-2Nal-THP-K(NMeAc)—N-3Pya-Sar-CON(Me)2;
cPEG3aCO-Pen(3)—N-T-7MeW—K(Ac)-Pen(3)—F(4Tz1TMA3)-2Nal-THP—K(Ac)—N-3Pya-Sar-CONH2;
cPEG3aCO-Pen(3)—N(N(Me)2)-T-7MeW—K(NMeAc)-Pen(3)—F(4Tz1TMA3)-2Nal-THP-K(NMeAc)—N-3Pya-Sar-CON(Me)2;
cPEG3aCO-Pen(3)—N-T-7MeW—K(Ac)-Pen(3)—F(4Tz1TMA4)-2Nal-THP—K(Ac)—N-3Pya-Sar-CONH2;
cPEG3aCO-Pen(3)—N(N(Me)2)-T-7MeW—K(NMeAc)-Pen(3)—F(4Tz1TMA4)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4Tz1TMA5)-2Nal-THP-K(NMeAc)—N-3Pya-Sar-CON(Me)2;
cPEG3aCO-Pen(3)—N(N(Me)2)-T-7MeW—K(NMeAc)-Pen(3)-GPEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-hFTMAPF-2Nal-THIP-K(NMeAc)—N-3Pya-Sar-CON(Me)2;
cPEG3aCO-Pen(3)—N(N(Me)2)-T-7MeW—K(NMeAc)-Pen(3)-MMoEF-2Nal-THP-K(NMeAc)—N-3Pya-Sar-CON(Me)2;
cPEG3aCO-Pen(3)—N(N(Me)2)-T-7(3NAcPh)W—K(NMeAc)-Pen(3)-MMoEF-2Nal-THP-K(NMeAc)—N-3Pya-Sar-CON(Me)2;
cPEG3aCO-Pen(3)—N(N(Me)2)-T-7MeW—K(NMeAc)-Pen(3)-MMoPF-2Nal-THIP-K(NMeAc)—N-3Pya-Sar-CON(Me)2;
cPEG3aCO-Pen(3)—N(N(Me)2)-T-7MeW—K(NMeAc)-Pen(3)-MMPEG3F-2Nal-THIP-K(NMeAc)—N-3Pya-Sar-CON(Me)2;
cPEG3aCO-Pen(3)—N(N(Me)2)-T-7MeW—K(NMeAc)-Pen(3)-morfTMA4F-2Nal-THIP-K(NMeAc)—N-3Pya-Sar-CON(Me)2;
cPEG3aCO-Pen(3)—N(N(Me)2)-T-7MeW—K(NMeAc)-Pen(3)-mPEG2TMA2F-2Nal-THIP-K(NMeAc)—N-3Pya-Sar-CON(Me)2;
cPEG3aCO-Pen(3)—N(N(Me)2)-T-7MeW—K(NMeAc)-Pen(3)-mPEG2TMA4F-2Nal-THIP-K(NMeAc)—N-3Pya-Sar-CON(Me)2;
cPEG3aCO-Pen(3)—N(N(Me)2)-T-7MeW—K(NMeAc)-Pen(3)-mPEG3 TMA4F-2Nal-THP-K(NMeAc)—N-3Pya-Sar-CON(Me)2;
cPEG3aCO-Pen(3)—N(N(Me)2)-T-7MeW—K(NMeAc)-Pen(3)-MPzPEG3F-2Nal-THIP-K(NMeAc)—N-3Pya-Sar-CON(Me)2;
cPEG3aCO-Pen(3)—N(N(Me)2)-T-7MeW—K(NMeAc)-Pen(3)-MTASF-2Nal-THP-K(NMeAc)—N-3Pya-Sar-CON(Me)2;
cPEG3aCO-Pen(3)—N(N(Me)2)-T-7MeW—K(NMeAc)-Pen(3)-NPyEF-2Nal-THIP-K(NMeAc)—N-3Pya-Sar-CON(Me)2;
cPEG3aCO-Pen(3)—N(N(Me)2)-T-7MeW—K(NMeAc)-Pen(3)-NPyPEG3F-2Nal-THIP-K(NMeAc)—N-3Pya-Sar-CON(Me)2;
cPEG3aCO-Pen(3)—N(N(Me)2)-T-7MeW—K(NMeAc)-Pen(3)-PiperazinequatF-2Nal-THP-K(NMeAc)—N-3Pya-Sar-CON(Me)2;
cPEG3aCO-Pen(3)—N(N(Me)2)-T-7MeW—K(NMeAc)-Pen(3)-TBAPEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-TMA3F-2Nal-THP-K(NMeAc)—N-3Pya-Sar-CON(Me)2;
cPEG3aCO-Pen(3)—N(N(Me)2)-T-7MeW—K(NMeAc)-Pen(3)-TMA3F-2Nal-THP-K(NMeAc)—N-3Pya-Sar-CON(Me)2;
cPEG3aCO-Pen(3)—N(N(Me)2)-T-7MeW—K(NMeAc)-Pen(3)-TMA4F-2Nal-THP-K(NMeAc)—N-3Pya-Sar-CON(Me)2;
cPEG3aCO-Pen(3)—N(N(Me)2)-T-7MeW—K(NMeAc)-Pen(3)-TMA6F-2Nal-THP-K(NMeAc)—N-3Pya-Sar-CON(Me)2;
cPEG3aCO-Pen(3)—N(N(Me)2)-T-7MeW—K(NMeAc)-Pen(3)-TMA8F-2Nal-THP-K(NMeAc)—N-3Pya-Sar-CON(Me)2;
cPEG3aCO-Pen(3)—K(cPEG3a)-T-7MeW—K(NMeAc)-Pen(3)-TMAPF-2Nal-THP-K(NMeAc)—N-3Pya-Sar-CON(Me)2;
cPEG3aCO-Pen(3)—K(Me)₃-T-7MeW—K(NMeAc)-Pen(3)-TMAPF-2Nal-THP-K(NMeAc)—N-3Pya-Sar-CON(Me)2;
cPEG3aCO-Pen(3)—K(Me)₃-T-7MeW—K(NMeAc)-Pen(3)-TMAPF-2Nal-Achx-K(NMeAc)—N-3Pya-Sar-CON(Me)2;
cPEG3aCO-Pen(3)—K(PEG2PEG2gEC12)-T-7MeW—K(Ac)-Pen(3)-TMAPF-2Nal-THP-K(NMeAc)—N-3Pya-NMeK(PEG2PEG2gEC12)—CONH2;
cPEG3aCO-k(PEG2PEG2gEC12)-Pen(3)—K(PEG2PEG2gEC12)-T-7MeW—K(NMeAc)-Pen(3)-TMAPF-2Nal-THP-K(NMeAc)—N-3Pya-Sar-CON(Me)2;
cPEG3aCO-Pen(3)—K(PEG2PEG2gEC12)-T-7MeW—K(NMeAc)-Pen(3)-TMAPF-2Nal-THP-K(NMeAc)—N-3Pya-Sar-CON(Me)2;
cPEG3aCO-Pen(3)—K(PEG2PEG2gEC12)-T-7MeW—K(PEG2PEG2gEC12)-Pen(3)-TMAPF-2Nal-THP-K(NMeAc)—N-3Pya-Sar-CON(Me)2;
cPEG3aCO-Pen(3)—K(PEG2PEG2gEC12)-T-7MeW—K(NMeAc)-Pen(3)-TMAPF-2Nal-THP-K(PEG2PEG2gEC12)—N-3Pya-Sar-CON(Me)2;
cPEG3aCO-Pen(3)—K(PEG2PEG2gEC14)-T-7MeW—K(Ac)-Pen(3)-TMAPF-2Nal-THP-K(NMeAc)—N-3Pya-NMeK(PEG2PEG2gEC14)—CONH2;
cPEG3aCO-k(PEG2PEG2gEC14)-Pen(3)—K(PEG2PEG2gEC14)-T-7MeW—K(NMeAc)-Pen(3)-TMAPF-2Nal-THP-K(NMeAc)—N-3Pya-Sar-CON(Me)2;
cPEG3aCO-Pen(3)—K(PEG2PEG2gEC14)-T-7MeW—K(NMeAc)-Pen(3)-TMAPF-2Nal-THP-K(NMeAc)—N-3Pya-Sar-CON(Me)2;
cPEG3aCO-Pen(3)—K(PEG2PEG2gEC14)-T-7MeW—K(PEG2PEG2gEC14)-Pen(3)-TMAPF-2Nal-THP-K(NMeAc)—N-3Pya-Sar-CON(Me)2;
cPEG3aCO-Pen(3)—K(PEG2PEG2gEC14)-T-7MeW—K(NMeAc)-Pen(3)-TMAPF-2Nal-THP-K(PEG2PEG2gEC14)—N-3Pya-Sar-CON(Me)2;
cPEG3aCO-Pen(3)—N-T-7MeW—K(NMeAc)-Pen(3)-TMAPF-2Nal-THP-K(NMeAc)—N-3Pya-Sar-CONH2;
cPEG3aCO-Pen(3)—N-T-7MeW—K(NMeAc)-Pen(3)-TMAPF-2Nal-THP-K(NMeAc)—N-3Pya-Sar-CON(Me)2;
cPEG3aCO-Pen(3)—N-T-7MeW—K(NMeAC)-Pen(3)-TMAPF-2Nal-THP-Dab(NMeAc)—N-3Pya-Sar-CON(Me)2;
cPEG3aCO-Pen(3)—N-T-7MeW-Dab(NMeAc)-Pen(3)-TMAPF-2Nal-THP-K(NMeAC)—N-3Pya-Sar-CON(Me)2;
cPEG3aCO-Pen(3)—N-T-7MeW—K(Ac)-Pen(3)-TMAPF-2Nal-THP-A-N-3Pya-Sar-CON(Me)2;
cPEG3aCO-Pen(3)—N-T-7MeW—K(Ac)-Pen(3)-TMAPF-2Nal-THP-K(PEG2PEG2gEC12)—N-3Pya-NMeK(PEG2PEG2gEC12)—CONH2;
cPEG3aCO-Pen(3)—N-T-7MeW—K(Ac)-Pen(3)-TMAPF-2Nal-THP-K(NMeAc)—N-3Pya-NMeK(PEG2PEG2gEC12)—CONH2;
cPEG3aCO-Pen(3)—N-T-7MeW—K(Ac)-Pen(3)-TMAPF-2Nal-THP-E-N-3Pya-Sar-CONH2;
cPEG3aCO-Pen(3)—N-T-7MeW—K(Ac)-Pen(3)-TMAPF-2Nal-THP—K(Ac)—N-3Pya-Sar-CONH2;
cPEG3aCO-Pen(3)—N-T-7(3NAcPh)W—K(Ac)-Pen(3)-TMAPF-2Nal-THP-E-N-3Pya-Sar-CONH2;
cPEG3aCO-Pen(3)—N(N(Me))-T-7MeW—K(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-THP-E-N-3Pya-Sar-CON(Me)2;
cPEG3aCO-Pen(3)—N(N(Me)2)-T-7MeW—K(NMeAc)-Pen(3)-TMAPF-2Nal-THP-K(NMeAc)—N-3Pya-Sar-CO(NHPEG3a);
cPEG3aCO-Pen(3)—N(N(Me)2)-T-7MeW-APEG2Ser(S*)-Pen(3)-TMAPF-2Nal-THP-K(NMeAc)—N-3Pya-Sar-CON(Me)2;
cPEG3aCO-Pen(3)—N(N(Me)2)-T-7MeW-APEG2Ser(R*)-Pen(3)-TMAPF-2Nal-THP-K(NMeAc)—N-3Pya-Sar-CON(Me)2;
cPEG3aCO-Pen(3)—N(N(Me)2)-T-7(3NAcPh)W—K(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(NMeAC)-Pen(3)-TMAPF-2Nal-THP-Dab(NMeAc)—N-3Pya-Sar-CON(Me)2;
cPEG3aCO-Pen(3)—N(N(Me)2)-T-7MeW—K(NMeAC)-Pen(3)-TMAPF-2Nal-THP-K(NMeAC)—N-3Pya-NMeK(PEG2PEG2C12)—CONH2;
cPEG3aCO-Pen(3)—N(N(Me)2)-T-7MeW—K(Ac)-Pen(3)-TMAPF-2Nal-THP-Dab(NMeAc)—N-3Pya-Sar-CON(Me)2;
cPEG3aCO-Pen(3)—N(N(Me)2)-T-7MeW—K(NMeAC)-Pen(3)-TMAPF-2Nal-THP-Q(N(Me)2)—N-3Pya-Sar-CON(Me)2;
cPEG3aCO-Pen(3)—N(N(Me)2)-T-7MeW-Q(N(Me)2)-Pen(3)-TMAPF-2Nal-THP-K(NMeAC)—N-3Pya-Sar-CON(Me)2;
cPEG3aCO-Pen(3)—N(N(Me)2)-T-7MeW—K(Ac)-Pen(3)-TMAPF-2Nal-THP-K(NMeAC)—N-3Pya-NMeK(PEG2PEG2gEC12)—CONH2;
cPEG3aCO-Pen(3)—N(N(Me)2)-T-7MeW—K(NMeAC)-Pen(3)-TMAPF-2Nal-THP-K(NMeAC)—N-3Pya-NMeK(PEG2PEG2gEC12)—CONH2;
cPEG3aCO-Pen(3)—N(N(Me)2)-T-7MeW—K(NMeAC)-Pen(3)-TMAPF-2Nal-THP-K(NMeAC)—N-3Pya-NMeK(PEG2PEG2gEC14)—CONH2;
cPEG3aCO-Pen(3)—N(N(Me)2)-T-7MeW—K(NMeAc)-Pen(3)-TMAPF-2Nal-THP-A-N-3Pya-Sar-CON(Me)2;
cPEG3aCO-Pen(3)—N(N(Me)2)-T-7MeW—K(NMeAc)-Pen(3)-TMAPF-2Nal-THP-K(NMeAc)—N-3Pya-Sar-CONH2;
cPEG3aCO-Pen(3)—N(N(Me)2)-T-7MeW—K(NMeAc)-Pen(3)-TMAPF-2Nal-THP-Cit-N-3Pya-Sar-CON(Me)2;
cPEG3aCO-Pen(3)—N(N(Me)2)-T-7MeW—K(Me)₃-Pen(3)-TMAPF-2Nal-THP-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-Acpx-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-Achx-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-Chg-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-Aib-K(NMeAc)—N-3Pya-Sar-CON(Me)2;
cPEG3aCO-Pen(3)—N(N(Me)2)-T-7MeW-Cit-Pen(3)-TMAPF-2Nal-THP-K(NMeAc)—N-3Pya-Sar-CON(Me)2;
cPEG3aCO-k(PEG2PEG2gEC14)-Pen(3)—N(N(Me)2)-T-7MeW—K(Ac)-Pen(3)-TMAPF-2Nal-THP-K(NMeAc)—N-3Pya-NMeK(PEG2PEG2gEC14)—CONH2;
cPEG3aCO-k(PEG2PEG2gEC12)-Pen(3)—N(N(Me)2)-T-7MeW—K(Ac)-Pen(3)-TMAPF-2Nal-THP-K(NMeAc)—N-3Pya-NMeK(PEG2PEG2gEC12)—CONH2;
cPEG3aCO-Pen(3)—N(N(Me)2)-T-7MeW—K(Ac)-Pen(3)-TMAPF-2Nal-THP-K(PEG2PEG2gEC14)—N-3Pya-NMeK(PEG2PEG2gEC14)—CONH2;
cPEG3aCO-Pen(3)—N(N(Me)2)-T-7MeW—K(PEG2PEG2gEC12)-Pen(3)-TMAPF-2Nal-THP-K(NMeAc)—N-3Pya-NMeK(PEG2PEG2gEC12)—CONH2;
cPEG3aCO-Pen(3)—N(N(Me)2)-T-7MeW—K(Ac)-Pen(3)-TMAPF-2Nal-THP-K(PEG2PEG2gEC12)—N-3Pya-NMeK(PEG2PEG2gEC12)—CONH2;
cPEG3aCO-Pen(3)—N(N(Me)2)-T-7MeW—K(PEG2PEG2gEC14)-Pen(3)-TMAPF-2Nal-THP-K(NMeAc)—N-3Pya-NMeK(PEG2PEG2gEC14)—CONH2;
cPEG3aCO-Pen(3)—N(N(Me)2)-T-7MeW-Dab(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG2C14)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG6gEC12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG6gEC14)—CONH2;
cPEG3aCO-Pen(3)—N(N(Me)2)-T-7MeW-Dab(NMeAc)-Pen(3)-TMAPF-2Nal-THP-K(NMeAc)—N-3Pya-Sar-CONH2;
cPEG3aCO-k(PEG2PEG6gEC12)-Pen(3)—N(N(Me)2)-T-7MeW—K(Ac)-Pen(3)-TMAPF-2Nal-THP-K(NMeAc)—N-3Pya-NMeK(PEG2PEG6gEC12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG2K(PEG2PEG2gEC12)₂)—CONH2;
cPEG3aCO-Pen(3)—N(N(Me)2)-T-7MeW—K(Ac)-Pen(3)-TMAPF-2Nal-THP-K(PEG2PEG2PEG2gEC12))—N-3Pya-NMeK(PEG2PEG2PEG2gEC12)—CONH2;
cPEG3aCO-k(PEG2PEG2gEC12)-Pen(3)—N(N(Me)2)-T-7MeW—K(NMeAc)-Pen(3)-TMAPF-2Nal-THP-K(NMeAc)—N-3Pya-Sar-CON(Me)2;
cPEG3aCO-k(PEG2PEG2gEC14)-Pen(3)—N(N(Me)2)-T-7MeW—K(NMeAc)-Pen(3)-TMAPF-2Nal-THP-K(NMeAc)—N-3Pya-Sar-CON(Me)2;
cPEG3aCO-k(PEG2PEG2gEC12)-Pen(3)—N(N(Me)2)-T-7MeW—K(PEG2PEG2gEC12)-Pen(3)-TMAPF-2Nal-THP-K(NMeAc)—N-3Pya-Sar-CON(Me)2;
cPEG3aCO-Pen(3)—N(N(Me)2)-T-7MeW—K(cPEG3a)-Pen(3)-TMAPF-2Nal-THP-K(NMeAc)—N-3Pya-Sar-CON(Me)2;
cPEG3aCO-Pen(3)—N(N(Me)2)-T-7MeW—K(4cpg)-Pen(3)-TMAPF-2Nal-THP-K(NMeAc)—N-3Pya-Sar-CON(Me)2;
cPEG3aCO-k(PEG2PEG2gEC12)-Pen(3)—N(N(Me)2)-T-7MeW—K(NMeAc)-Pen(3)-TMAPF-2Nal-THP-K(PEG2PEG2gEC12)—N-3Pya-Sar-CON(Me)2;
cPEG3aCO-k(PEG2PEG2gEC14)-Pen(3)—N(N(Me)2)-T-7MeW—K(NMeAc)-Pen(3)-TMAPF-2Nal-THP-K(PEG2PEG2gEC14)—N-3Pya-Sar-CON(Me)2;
cPEG3aCO-Pen(3)—N(N(Me)2)-T-7MeW—K(PEG2PEG2gEC14)-Pen(3)-TMAPF-2Nal-THP-K(NMeAc)—N-3Pya-Sar-CON(Me)2;
cPEG3aCO-Pen(3)—N(N(Me)2)-T-7MeW—K(PEG2PEG2gEC12)-Pen(3)-TMAPF-2Nal-THP-K(PEG2PEG2gEC12)—N-3Pya-Sar-CON(Me)2;
cPEG3aCO-Pen(3)—N(N(Me)2)-T-7MeW—K(NMeAc)-Pen(3)-TMAPF-2Nal-THP-K(PEG2PEG2gEC12)—N-3Pya-Sar-CON(Me)2;
cPEG3aCO-Pen(3)—N(N(Me)2)-T-7MeW—K(NMeAc)-Pen(3)-TMAPF-2Nal-THP-K(PEG2PEG2gEC14)—N-3Pya-Sar-CON(Me)2;
cPEG3aCO-k(PEG2PEG2PEG2PEG2gEC12)-Pen(3)—N(N(Me)2)-T-7MeW—K(Ac)-Pen(3)-TMAPF-2Nal-THP-K(NMeAc)—N-3Pya-NMeK(PEG2PEG2PEG2PEG2gEC12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(SP6PEG2PEG2C12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(SP6PEG2PEG2gEC12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG2K(PEG2PEG2PEG2PEG2gEC12)₂)—CONH2;
cPEG3aCO-Pen(3)—N(N(Me)2)-T-7MeW—K(PEG2PEG2gEC14)-Pen(3)-TMAPF-2Nal-THP-K(PEG2PEG2gEC14)—N-3Pya-Sar-CON(Me)2;
cPEG3aCO-Pen(3)—N(N(Me)2)-T-7MeW-Dap(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(Ac)-Pen(3)-TMAPF-2Nal-THP-Dap(NMeAc)—N-3Pya-Sar-CON(Me)2;
cPEG3aCO-Pen(3)—N(N(Me)2)-T-7MeW—K(NMeAc)-Pen(3)-Tzl(Ch)-2Nal-THP-K(NMeAc)—N-3Pya-Sar-CON(Me)2;
cPEG3aCO-Pen(3)—N(N(Me)2)-T-7MeW—K(NMeAc)-Pen(3)-TzlChmPEG-2Nal-THP-K(NMeAc)—N-3Pya-Sar-CON(Me)2;
cPEG3aCO-Pen(3)—N(N(Me)2)-T-7MeW—K(NMeAc)-Pen(3)-TzlChmPEG3-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(C9OH)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OEOXIMECh)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OTzlCh)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OTzlChC16)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OTzlC1aC8)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OTzlChmPEG)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OTzlChmPEG3)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OTz1TMA4)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OTz1TMA4)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OZOXIMECh)-2Nal-THP-K(NMeAc)—N-3Pya-Sar-CON(Me)2;
cPEG3aCO-Pen(3)—N(N(Me)2)-T-7MeW—K(NMeAc)-Pen(3)—YC8CO(NHPEG3a)-2Nal-THP-K(NMeAc)—N-3Pya-Sar-CON(Me)2;
cPEG3aCO-Pen(3)—N(N(Me)2)-T-7MeW—K(NMeAc)-Pen(3)-YC8COPip-2Nal-THP-K(NMeAc)—N-3Pya-Sar-CON(Me)2;
cPEG3gCO-Pen(3)—N(N(Me)2)-T-7MeW—K(NMeAc)-Pen(3)-APEG3F-2Nal-THP-K(NMeAc)—N-3Pya-Sar-CON(Me)2;
cPEG3gCO-Pen(3)—N(N(Me)2)-T-7MeW—K(NMeAc)-Pen(3)-TMAPF-2Nal-THP-K(NMeAc)—N-3Pya-Sar-CON(Me)2;
   or a pharmaceutically acceptable salt thereof.

In another aspect of the invention:

14. A compound of Formula (V)

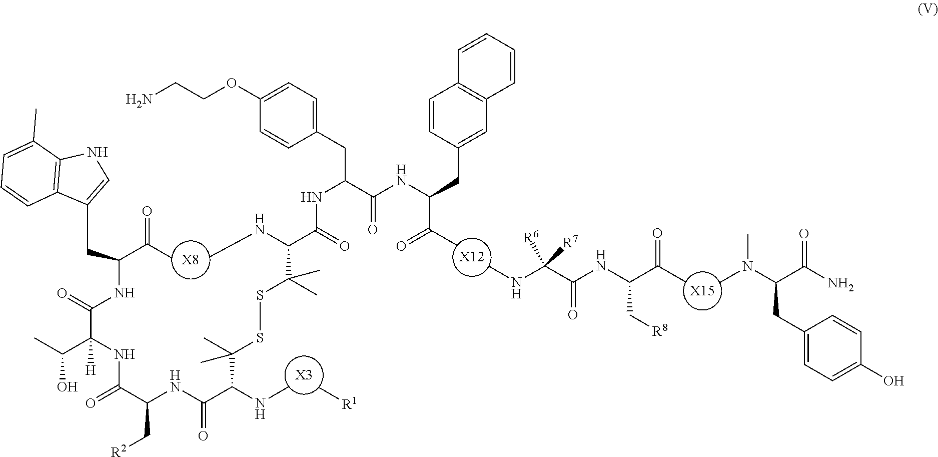

wherein:
R¹ is —C(O)C(1-2)alkyl, —C(O)CF₃, —COCH₂CF₃, —CO(CH₂)₅N(CH₃)₃+AN⁻, —CH₂CH₂NHC(O)CH₂O(CH₂CH₂O)₂CH₃, —CH₂CH₂NHC(O)(CH₂CH₂O)₆CH₃,

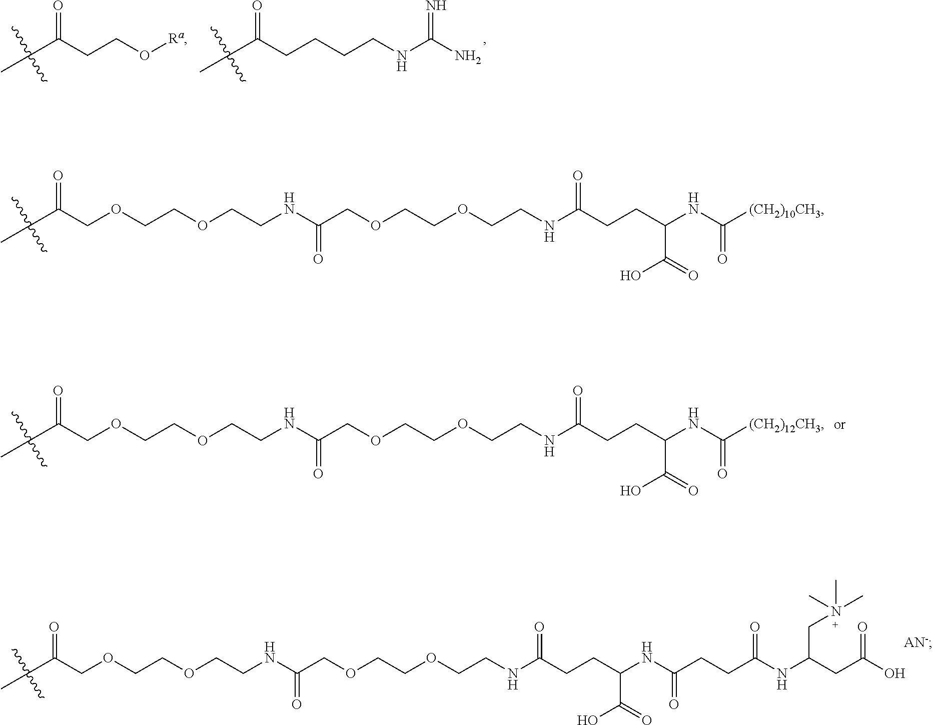

wherein:
R^{a} is

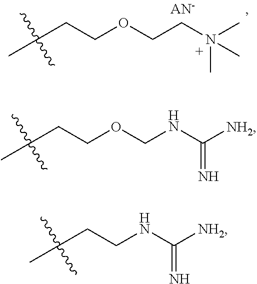

—CH₂CH₂OCH₂CH₂N(CH₃)₂,
—(CH₂CH₂O)₃CH₂CH₂N(CH₃)₂, —(CH₂CH₂O)₃CH₂CH₂N(CH₃)₃ ⁺AN⁻, —(CH₂CH₂O)₅CH₂CH₂NC(O)CH₃, —(CH₂CH₂O)₈CH₂CH₂NC(O)CH₃, or
X3 is a direct bond, (D) arginine, (D) lysine substituted with R^{b},

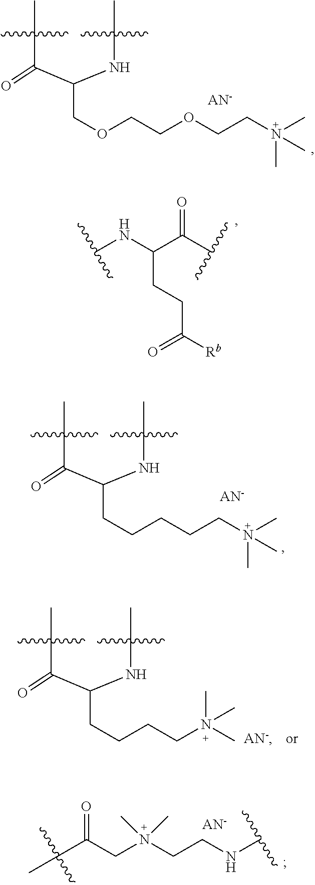

wherein:
   R^{b} is

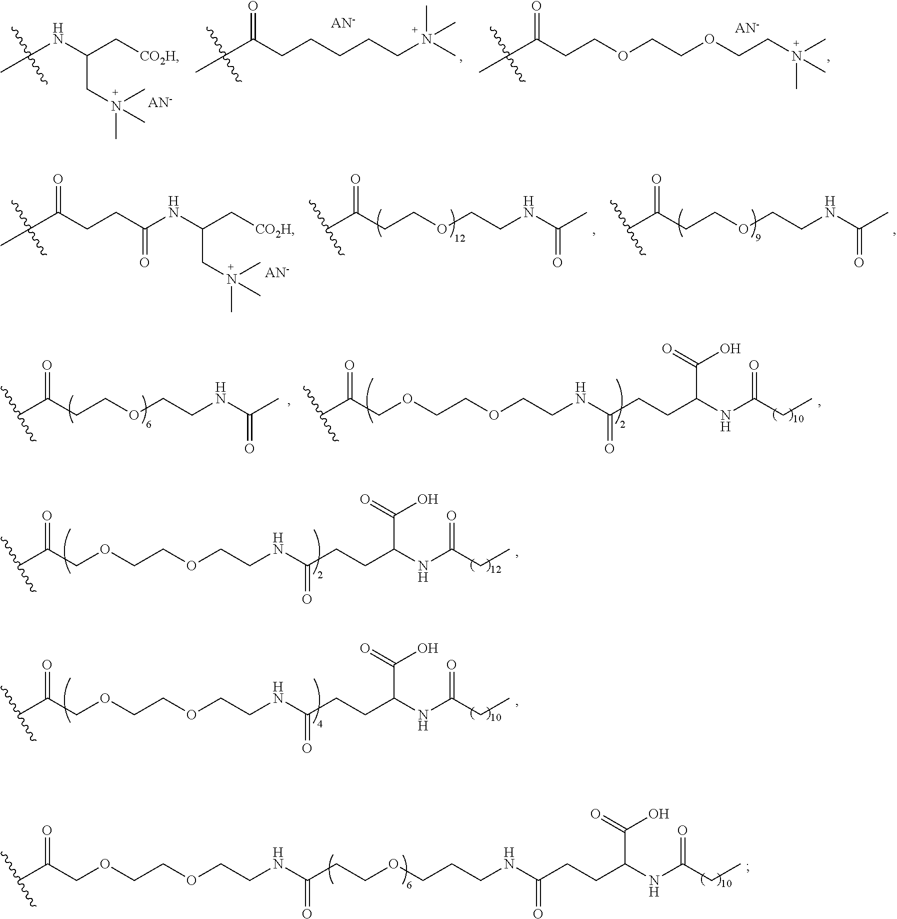

R² is H, —CH(CH₃)₂

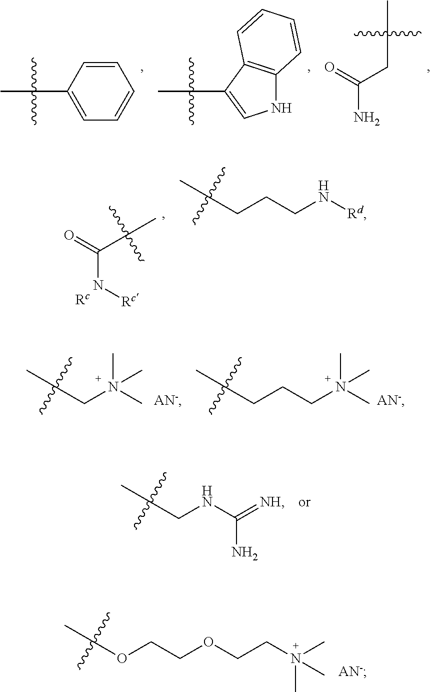

wherein:
   R^{c} is H, or —CH₃;
   R^{c}′ is H, or —CH₃;
   R^{d} is

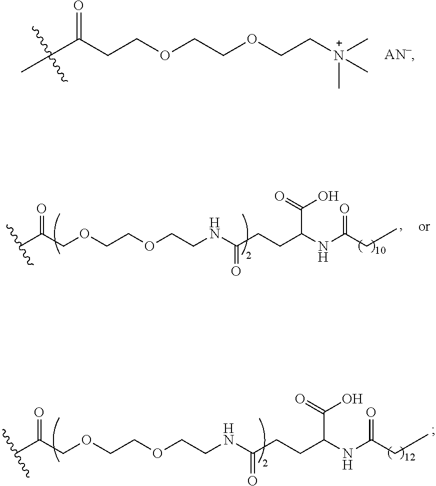

X8

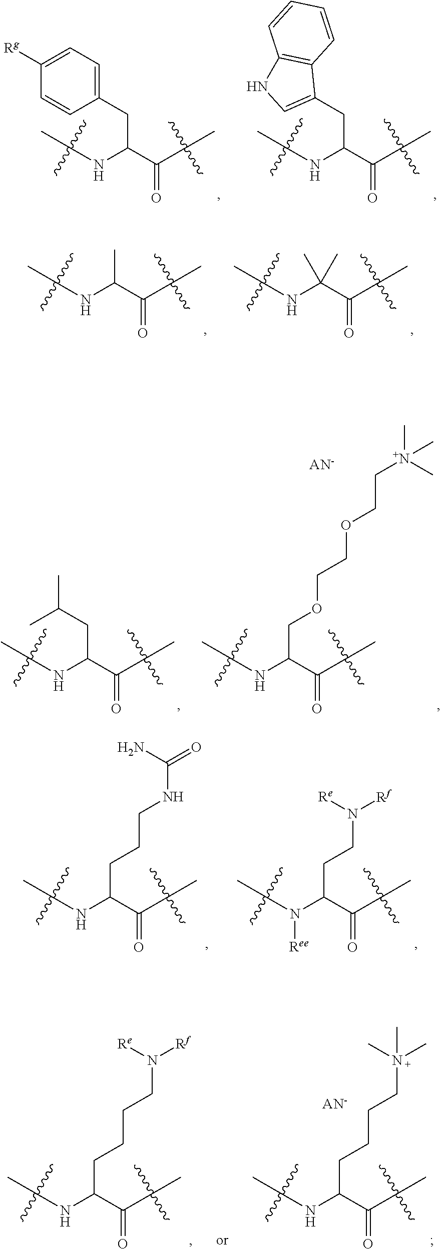

wherein:
   R^{e} is H, or —CH₃;
   R^{ee} is H;
   R^{f} is H, —CH₃, —C(CH₃)₃, —C(O)CH₃, —C(O)(CH₂CH₂O)₆CH₃, —(CH₂CH₂O)₆CH₃,

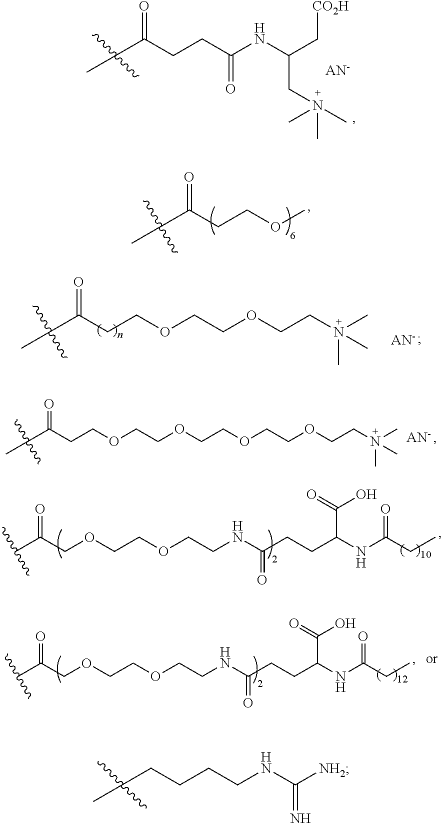

R^{g} is H, —OH, —C(O)NH₂, or —CH₂NHC(O)CH₃;
n is 0, or 1;
X12 is absent,

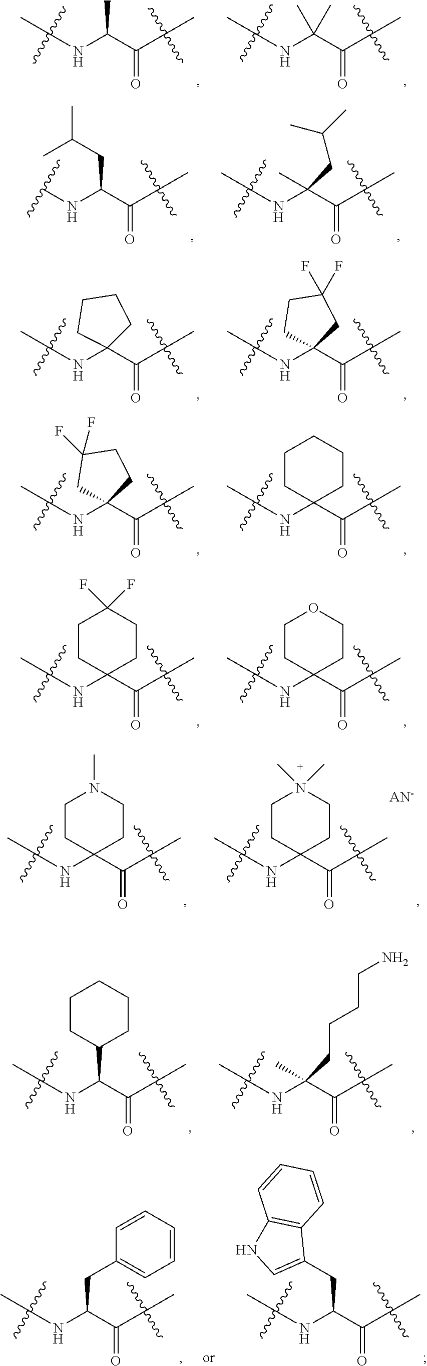

R⁶ is —CH₃, —CH₂CH₂C(O)R^{m}, —(CH₂)ₛNRⁿR^{o},

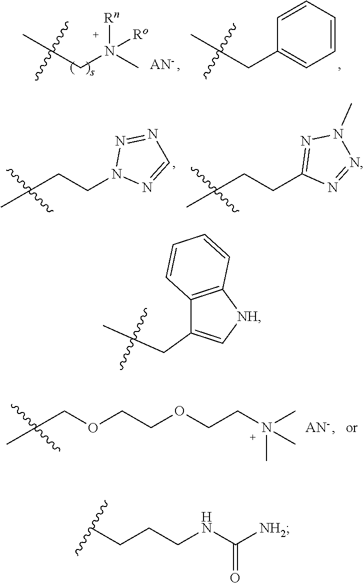

R⁷ is H, or —CH₃
   wherein:
      R^{m} is —OH, or

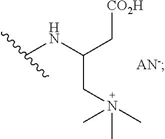

Rⁿ is H, —CH₃, —CH₂CH(CH₃)₂, —C(O)CH₃, —C(O)(CH₂CH₂O)₆CH₃, —(CH₂CH₂O ₆CHs, —C(O)(CH₂CH₂O)ₜCH₂CH₂R^{p},

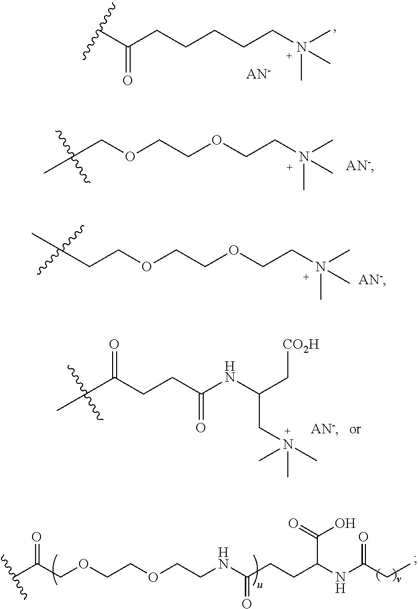

R^{o} is H, —CH₃, or —C(O)CH₃;
wherein:
   R^{p} is NHC(O)CH₃, or

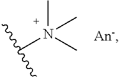

s is 1, 2, or 4;
t is 2, 6, 9, or 12;
u is 2 or 3;
v is 10 or 12;
R⁸ is —CH₂CH₂CH₂NHC(O)CH₃, —C(O)NH₂, or —C(O)NHCH₃;
X15 is absent,

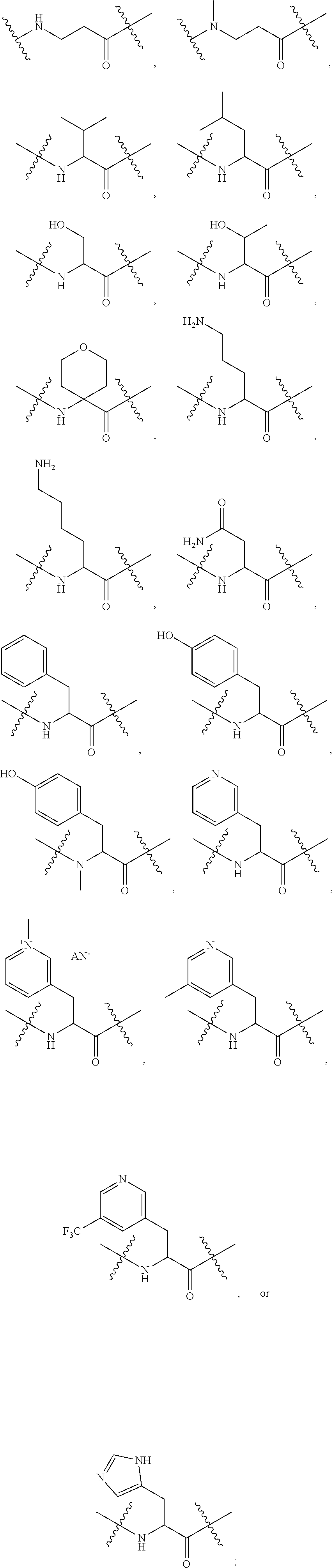

wherein:
AN⁻as above defined for A, R, X3, X12, R1, R2, R5, R6, R8, R15, R16 and R^{a}, R^{b}, R^{d}, R^{f}, R^{h}, Rⁱ, R^{j}, R^{k}, R^{m}, Rⁿ, R^{o}, R^{s} is acetate, adipate, benzoate, benzenesulfonate, citrate, decanoate, chloride, lactate, maleate, methanesulfonate, oxalate, pivalate, propionate, succinate, sulfate, tartrate, or trifluoroacetate; or
a pharmaceutically acceptable salt thereof.

In another aspect of the invention:

15. The compound of aspect 14 which is
MeCO-Pen(3)—N-T-7MeW—K(Ac)-Pen(3)-AEF-2Nal-aMeK—K(Ac)—N-dK-NMeDTyr-CONH2;
MeCO-Pen(3)—N-T-7MeW—K(Ac)-Pen(3)-AEF-2Nal-aMeK—K(Ac)—N-dL-NMeDTyr-CONH2;
MeCO-Pen(3)—N-T-7MeW—K(Ac)-Pen(3)-AEF-2Nal-aMeK—K(Ac)—N—F—NMeDTyr-CONH2;
MeCO-Pen(3)—N-T-7MeW—K(Ac)-Pen(3)-AEF-2Nal-aMeK—K(Ac)—N—H—NMeDTyr-CONH2;
MeCO-r-Pen(3)—N-T-7MeW—K(Ac)-Pen(3)-AEF-2Nal-aMeK-E-N—H—NMeDTyr-CONH2;
MeCO-Pen(3)—N-T-7MeW—K(Ac)-Pen(3)-AEF-2Nal-aMeK—K(Ac)—N-Paf-NMeDTyr-CONH2;
MeCO-r-Pen(3)—N-T-7MeW—K(Ac)-Pen(3)-AEF-2Nal-THP-E-N-h-NMeDTyr-CONH2;
MeCO-r-Pen(3)—N-T-7MeW—K(Ac)-Pen(3)-AEF-2Nal-THP-E-N—N—NMeDTyr-CONH2;
MeCO-Pen(3)—N-T-7MeW—K(Ac)-Pen(3)-AEF-2Nal-aMeK—K(Ac)—N—N—NMeDTyr-CONH2;
MeCO-Pen(3)—N-T-7MeW—K(Ac)-Pen(3)-AEF-2Nal-aMeK—K(Ac)—N-v-NMeDTyr-CONH2;
MeCO-Pen(3)—N-T-7MeW—K(Ac)-Pen(3)-AEF-2Nal-aMeK—K(Ac)—N-t-NMeDTyr-CONH2;
MeCO-r-Pen(3)—N-T-7MeW-Cit-Pen(3)-AEF-2Nal-aMeL-E-N-k-NMeDTyr-CONH2;
MeCO-r-Pen(3)—N-T-7MeW-Cit-Pen(3)-AEF-2Nal-aMeL-E-N-h-NMeDTyr-CONH2;
MeCO-Pen(3)—N-T-7MeW—K(Ac)-Pen(3)-AEF-2Nal-aMeK—K(Ac)—N-orn-NMeDTyr-CONH2;
MeCO-Pen(3)—N-T-7MeW—K(Ac)-Pen(3)-AEF-2Nal-aMeK—K(Ac)—N-s-NMeDTyr-CONH2;
MeCO-Pen(3)—N-T-7MeW—K(Ac)-Pen(3)-AEF-2Nal-aMeK—K(Ac)—N-f-NMeDTyr-CONH2;
MeCO-Pen(3)—N-T-7MeW—Y-Pen(3)-AEF-2Nal-aMeK—K(Ac)—N-k-NMeDTyr-CONH2;
MeCO-Pen(3)—N-T-7MeW-4AmPhe-Pen(3)-AEF-2Nal-aMeK—K(Ac)—N-k-NMeDTyr-CONH2;
MeCO-Pen(3)—N-T-7MeW-Paf(Ac)-Pen(3)-AEF-2Nal-aMeK—K(Ac)—N-k-NMeDTyr-CONH2;
MeCO-Pen(3)—N-T-7MeW—K(Ac)-Pen(3)-AEF-2Nal-aMeK—K(Ac)—N-y-NMeDTyr-CONH2;
MeCO-Pen(3)—N-T-7MeW-Cit-Pen(3)-AEF-2Nal-aMeL-K(Ac)—N-k-NMeDTyr-CONH2;
MeCO-Pen(3)—N-T-7MeW—K(Ac)-Pen(3)-AEF-2Nal-aMeK—K(Ac)—N-h-NMeDTyr-CONH2;
MeCO-Pen(3)—N-T-7MeW—K(Ac)-Pen(3)-AEF-2Nal-aMeK—K(Ac)—N-bAla-NMeDTyr-CONH2;
MeCO-Pen(3)—N-T-7MeW—K(Ac)-Pen(3)-AEF-2Nal-aMeK—K(Ac)—N-bAla-NMeDTyr-CONH2;
MeCO-Pen(3)—N-T-7MeW—K(Ac)-Pen(3)-AEF-2Nal-aMeK—K(Ac)—N-bAla-NMeDTyr-CONH2;
MeCO-Pen(3)—N-T-7MeW—K(Ac)-Pen(3)-AEF-2Nal-aMeK—K(Ac)—N-3Pya-NMeDTyr-CONH2;
MeCO-Pen(3)—N-T-7MeW—K(Ac)-Pen(3)-AEF-2Nal-aMeK—K(Ac)—N-3pya-NMeDTyr-CONH2;
MeCO-r-Pen(3)—N-T-7MeW—K(Ac)-Pen(3)-AEF-2Nal-THP-E-N-3Pya-NMeDTyr-CONH2;
MeCO-r-Pen(3)-Q-T-W-Q-Pen(3)-AEF-2Nal-THP-aMeE-N—F—NMeDTyr-CONH2;
MeCO-r-Pen(3)-Q-T-W-Q-Pen(3)-AEF-2Nal-THP-E-N—N—NMe DTyr-CONH2;
   or a pharmaceutically acceptable salt thereof.

In another aspect of the invention:

16. A compound of Formula (VI)

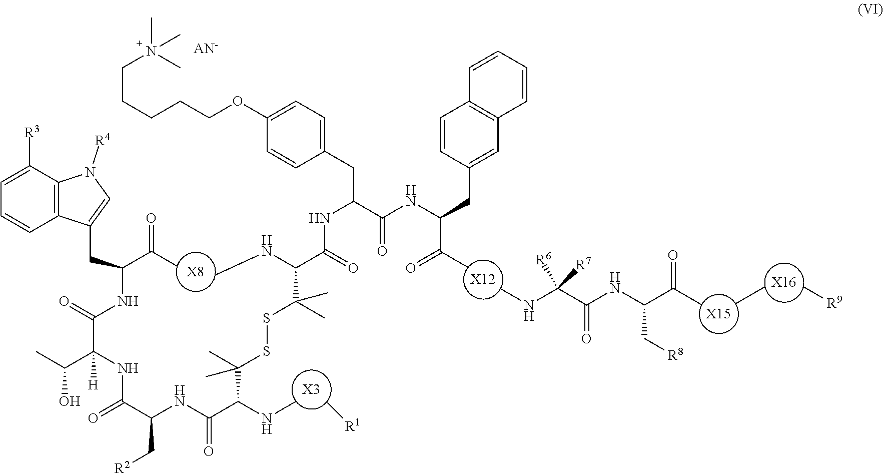

wherein:
R¹ is —C(O)C(1-2)alkyl, —C(O)CF₃, —COCH₂CF₃, —CO(CH₂)₅N(CH₃)₃⁺AN⁻, —CH₂CH₂NHC(O)CH₂O(CH₂CH₂O)₂CH₃, —CH₂CH₂NHC(O)(CH₂CH₂O)₆CH₃,

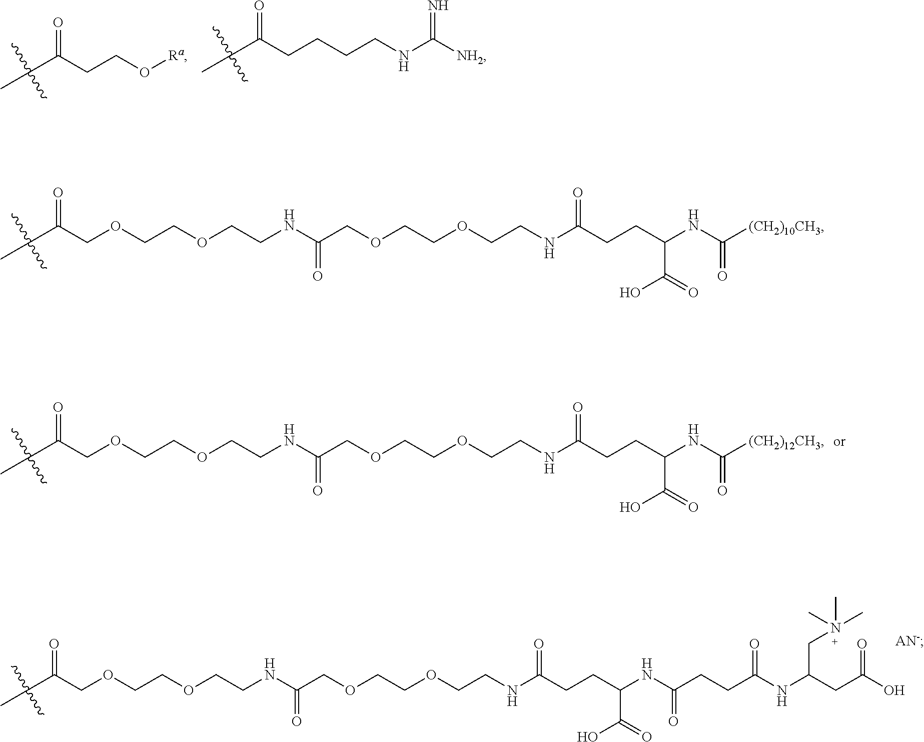

wherein:
Rₐ is

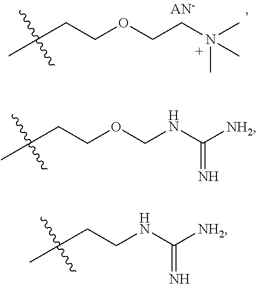

CH₂CH₂OCH₂CH₂N(CH₃)₂,
—(CH₂CH₂O)₃CH₂CH₂N(CH₃)₂, —(CH₂CH₂O)₃CH₂CH₂N(CH₃)₃ ⁺AN⁻, —(CH₂CH₂O)₅CH₂CH₂NC(O)CH₃, —(CH₂CH₂O)₈CH₂CH₂NC(O)CH₃, or —(CH₂CH₂O)₁₁CH₂CH₂NC(O)CH₃;
X3 is a direct bond, (D) arginine, (D) lysine substituted with R^{b},

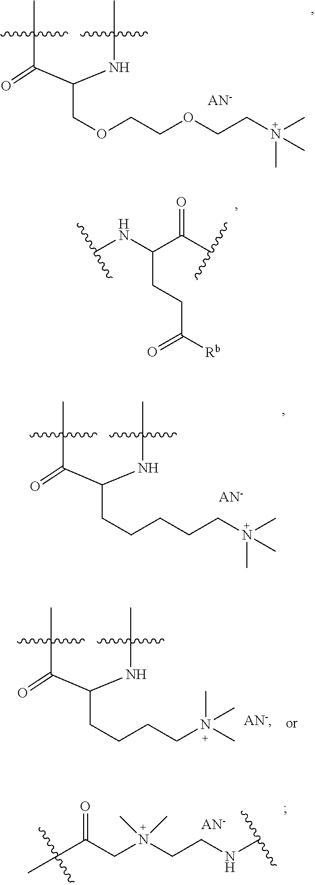

wherein:
R^{b} is

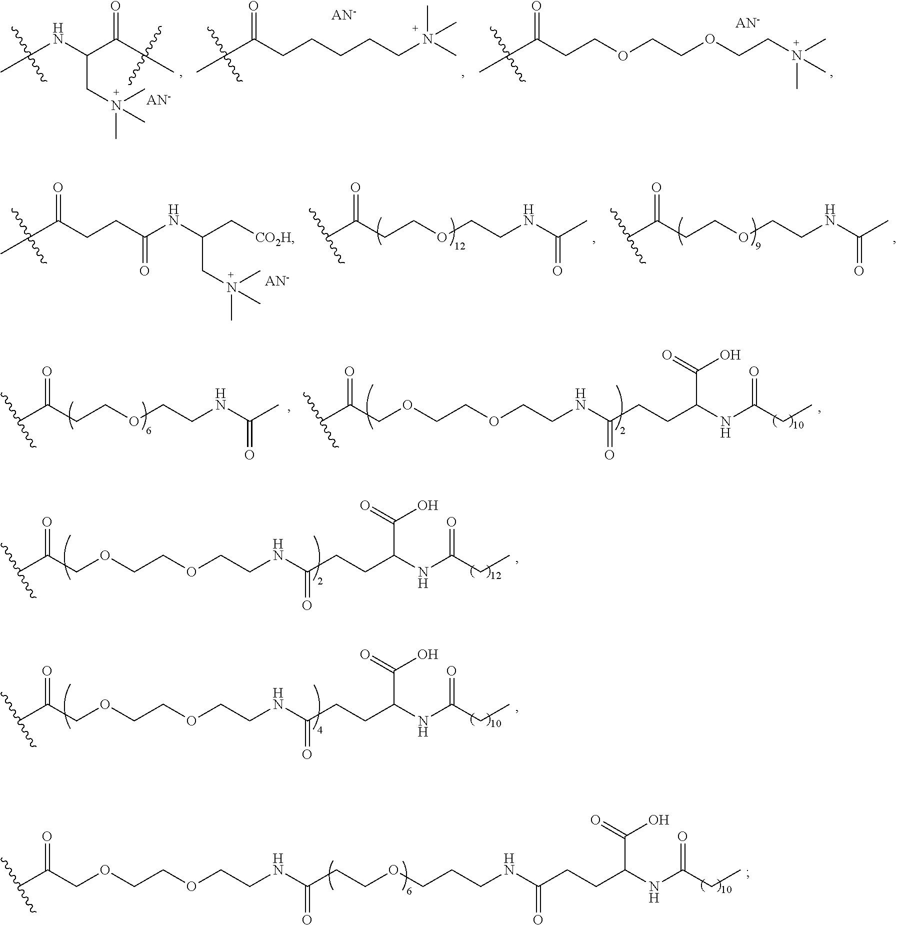

R² is H, —CH(CH₃)₂,

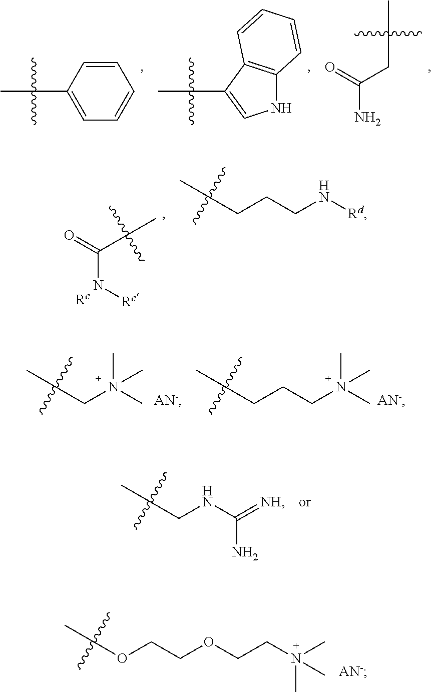

wherein:
   R^{c} is H, or —CH₃;
   R^{c}′ is H, or —CH₃;
   R^{d} is

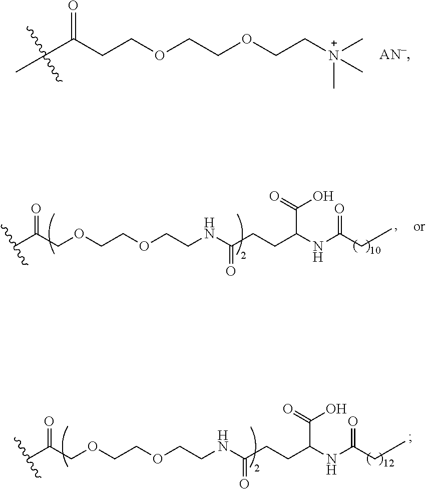

R³ is H, —CH₃, —CF₃, or

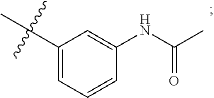 [SMILES: *c1cccc(NC(C)=O)c1]

R⁴ is H, or —CH₃;
X8 is

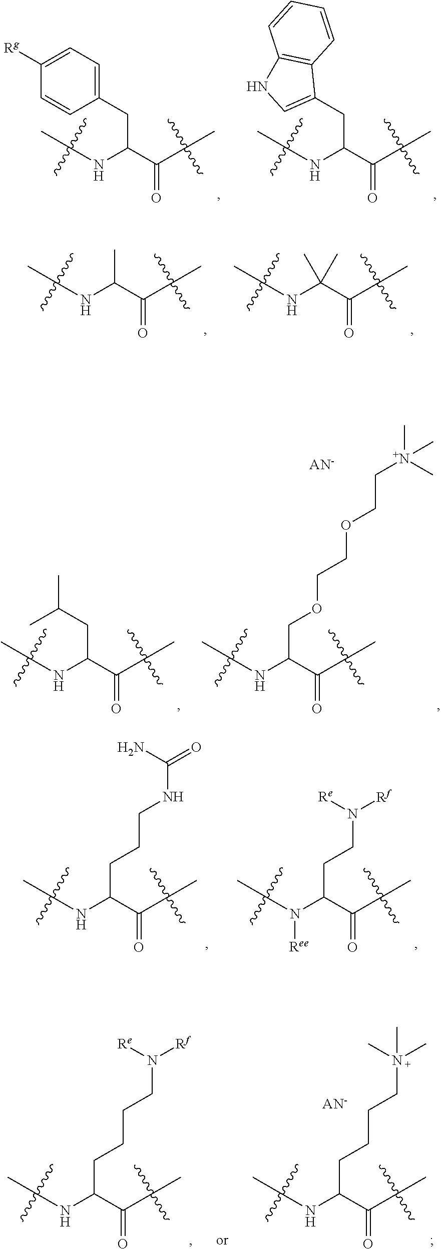

wherein:
   R^{e} is H, or —CH₃;
   R^{ee} is H;
   R^{f} is H, —CH₃, —C(CH₃)₃, —C(O)CH₃, —C(O)(CH₂CH₂O)₆CH₃, —(CH₂CH₂O)₆CH₃,

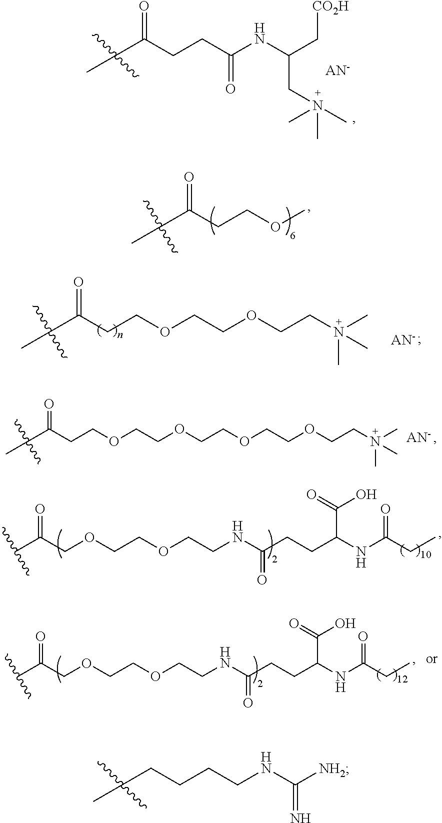

R^{g} is H, —OH, —C(O)NH₂, or —CH₂NHC(O)CH₃;
n is 0, or 1;
X12 is absent,

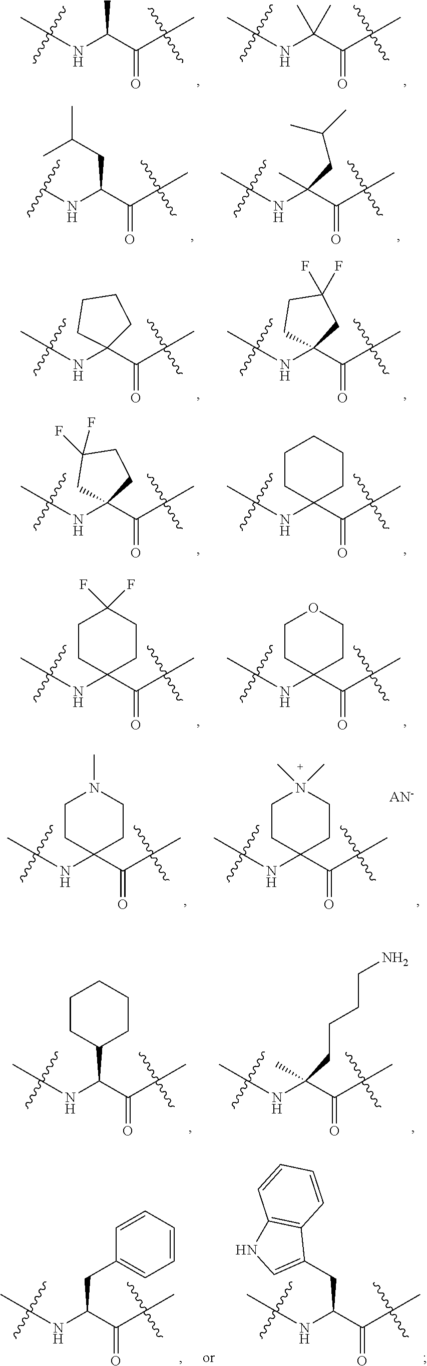

R⁶ is —CH₃, —CH₂CH₂C(O)R^{m}, —(CH₂)ₛNRⁿR^{o},

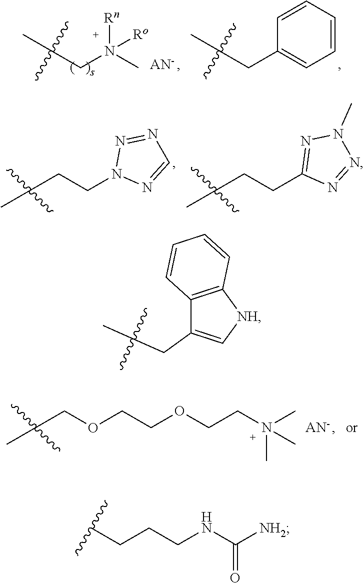

R⁷ is H, or —CH₃;
   wherein:
      R^{m} is —OH, or

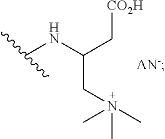

Rⁿ is H, —CH₃, —CH₂CH(CH₃)₂, —C(O)CH₃, —C(O)(CH₂CH₂O)₆CH₃, —(CH₂CH₂O)₆CH₃, —C(O)(CH₂CH₂O)ₜCH₂CH₂R^{p},

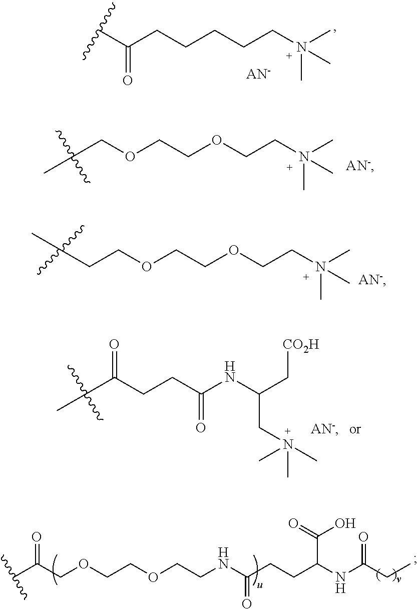

R^{o} is H, —CH₃, or —C(O)CH₃;
wherein:
   R^{p} is NHC(O)CH₃, or

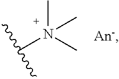

s is 1, 2, or 4;
t is 2, 6, 9, or 12;
u is 2 or 3;
v is 10 or 12;
R⁸ is —CH₂CH₂CH₂NHC(O)CH₃, —C(O)NH₂, or —C(O)NHCH₃;
X15 is absent,

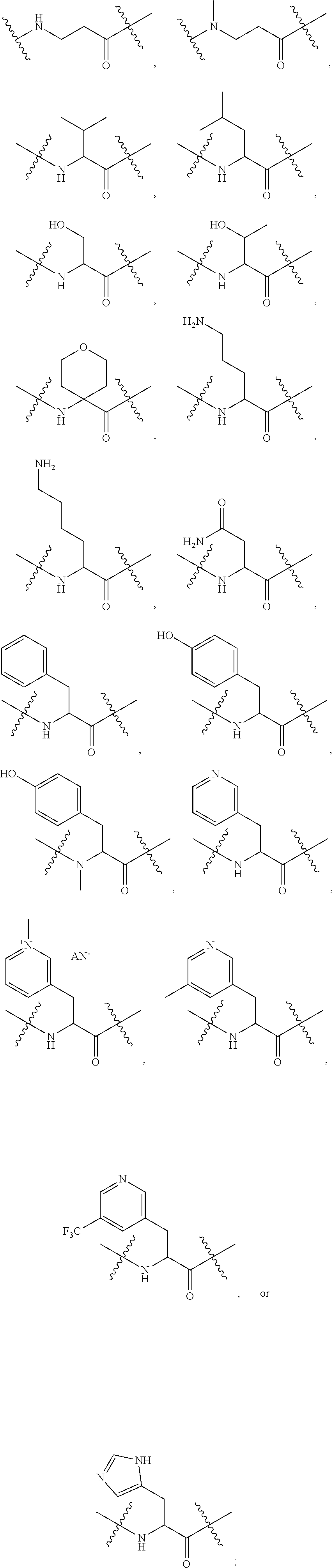

X16 is absent,

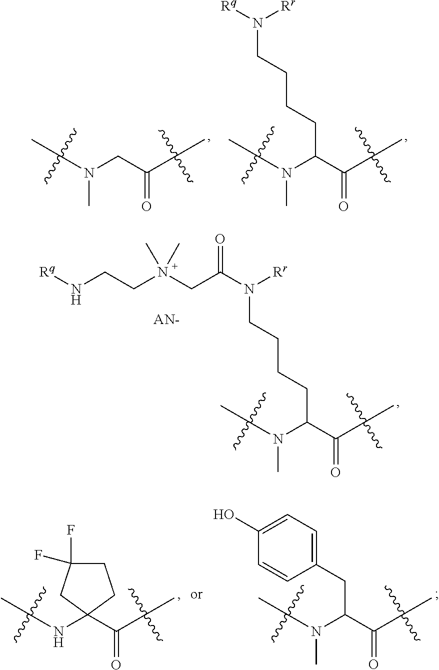

wherein:
   R^{q} is

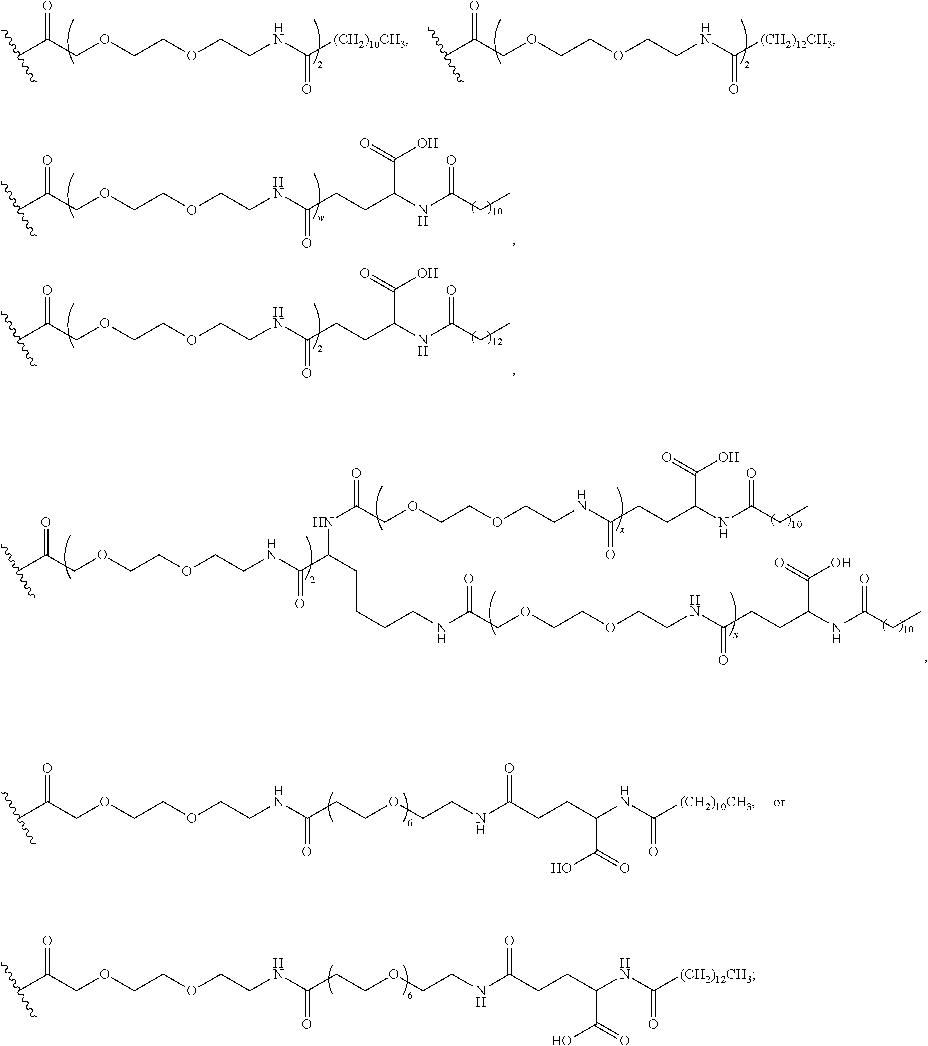

R^{r} is H;
w is 2 4, or 5;
x is 2 or N and
R⁹ is —NH₂, —NHCH₃, —N(CH₃)₂, —NH(CH₂CH₂O)₂CH₃, —N(CH₃)(CH₂CH₂O)₂CH₃, —NH(CH₂CH₂O)_{y}R^{s},

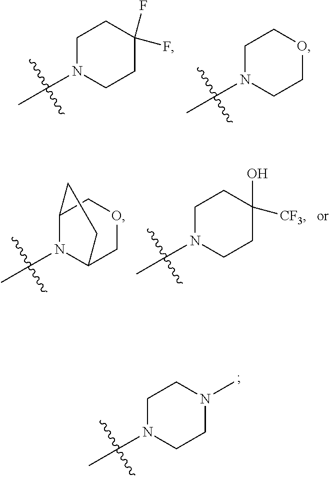

wherein:
   y is 2, 4, or 6;
R^{s} is —CH₃, or

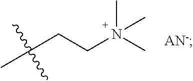

wherein:
AN⁻as above defined for A, R, X3, X12, R1, R2, R5, R6, R8, R15, R16 and R^{a}, R^{b}, R^{d}, R^{f}, R^{h}, Rⁱ, R^{j}, R^{k}, R^{m}, Rⁿ, R^{o}, R^{s} is acetate, adipate, benzoate, benzenesulfonate, citrate, decanoate, chloride, lactate, maleate, methanesulfonate, oxalate, pivalate, propionate, succinate, sulfate, tartrate, or trifluoroacetate; or
a pharmaceutically acceptable salt thereof.

In another aspect of the invention:

17. The compound of aspect 16 wherein
AN⁻is chloride;
or a pharmaceutically acceptable salt thereof.

In another aspect of the invention:

18. The compound of aspect 16 which is
cPEG3aCO-Pen(3)—N(N(Me)2)-T-7MeW—K(NMeAC)-Pen(3)-TMAPF-2Nal-THP-K(NMeAC)—N-3Pya-NMeK(PEG2PEG2C12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG2C14)—CONH2;
C12gEPEG2PEG2CO-Pen(3)—N(N(Me)2)-T-7MeW—K(Ac)-Pen(3)-TMAPF-2Nal-THP-K(NMeAc)—N-3Pya-NMeK(PEG2PEG2gEC12)—CONH2;
cPEG3aCO-Pen(3)—K(PEG2PEG2gEC12)-T-7MeW—K(Ac)-Pen(3)-TMAPF-2Nal-THP-K(NMeAc)—N-3Pya-NMeK(PEG2PEG2gEC12)—CONH2;
cPEG3aCO-Pen(3)—N-T-7MeW—K(Ac)-Pen(3)-TMAPF-2Nal-THP-K(PEG2PEG2gEC12)—N-3Pya-NMeK(PEG2PEG2gEC12)—CONH2;
cPEG3aCO-Pen(3)—N-T-7MeW—K(Ac)-Pen(3)-TMAPF-2Nal-THP-K(NMeAc)—N-3Pya-NMeK(PEG2PEG2gEC12)—CONH2;
cPEG3aCO-Pen(3)—N(N(Me)2)-T-7MeW—K(Ac)-Pen(3)-TMAPF-2Nal-THP-K(NMeAC)—N-3Pya-NMeK(PEG2PEG2gEC12)—CONH2;
cPEG3aCO-Pen(3)—N(N(Me)2)-T-7MeW—K(NMeAC)-Pen(3)-TMAPF-2Nal-THP-K(NMeAC)—N-3Pya-NMeK(PEG2PEG2gEC12)—CONH2;
cPEG3aCO-k(PEG2PEG2gEC12)-Pen(3)—N(N(Me)2)-T-7MeW—K(Ac)-Pen(3)-TMAPF-2Nal-THP-K(NMeAc)—N-3Pya-NMeK(PEG2PEG2gEC12)—CONH2;
cPEG3aCO-Pen(3)—N(N(Me)2)-T-7MeW—K(PEG2PEG2gEC12)-Pen(3)-TMAPF-2Nal-THP-K(NMeAc)—N-3Pya-NMeK(PEG2PEG2gEC12)—CONH2;
cPEG3aCO-Pen(3)—N(N(Me)2)-T-7MeW—K(Ac)-Pen(3)-TMAPF-2Nal-THP-K(PEG2PEG2gEC12)—N-3Pya-NMeK(PEG2PEG2gEC12)—CONH2;
C14gEPEG2PEG2CO-Pen(3)—N(N(Me)2)-T-7MeW—K(Ac)-Pen(3)-TMAPF-2Nal-THP-K(NMeAc)—N-3Pya-NMeK(PEG2PEG2gEC14)—CONH2;
cPEG3aCO-Pen(3)—K(PEG2PEG2gEC14)-T-7MeW—K(Ac)-Pen(3)-TMAPF-2Nal-THP-K(NMeAc)—N-3Pya-NMeK(PEG2PEG2gEC14)—CONH2;
cPEG3aCO-Pen(3)—N(N(Me)2)-T-7MeW—K(NMeAC)-Pen(3)-TMAPF-2Nal-THP-K(NMeAC)—N-3Pya-NMeK(PEG2PEG2gEC14)—CONH2;
cPEG3aCO-k(PEG2PEG2gEC14)-Pen(3)—N(N(Me)2)-T-7MeW—K(Ac)-Pen(3)-TMAPF-2Nal-THP-K(NMeAc)—N-3Pya-NMeK(PEG2PEG2gEC14)—CONH2;
cPEG3aCO-Pen(3)—N(N(Me)2)-T-7MeW—K(Ac)-Pen(3)-TMAPF-2Nal-THP-K(PEG2PEG2gEC14)—N-3Pya-NMeK(PEG2PEG2gEC14)—CONH2;
cPEG3aCO-Pen(3)—N(N(Me)2)-T-7MeW—K(PEG2PEG2gEC14)-Pen(3)-TMAPF-2Nal-THP-K(NMeAc)—N-3Pya-NMeK(PEG2PEG2gEC14)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG2K(PEG2PEG2gEC12)₂)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG2K(PEG2PEG2PEG2PEG2gEC12)₂)—CONH2;
cPEG3aCO-Pen(3)—N(N(Me)2)-T-7MeW—K(Ac)-Pen(3)-TMAPF-2Nal-THP-K(PEG2PEG2PEG2gEC12))—N-3Pya-NMeK(PEG2PEG2PEG2gEC12)—CONH2;
cPEG3aCO-k(PEG2PEG2PEG2PEG2gEC12)-Pen(3)—N(N(Me)2)-T-7MeW—K(Ac)-Pen(3)-TMAPF-2Nal-THP-K(NMeAc)—N-3Pya-NMeK(PEG2PEG2PEG2PEG2gEC12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG6gEC12)—CONH2;
cPEG3aCO-k(PEG2PEG6gEC12)-Pen(3)—N(N(Me)2)-T-7MeW—K(Ac)-Pen(3)-TMAPF-2Nal-THP-K(NMeAc)—N-3Pya-NMeK(PEG2PEG6gEC12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG6gEC14)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(SP6PEG2PEG2C12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(SP6PEG2PEG2gEC12)—CONH2;
4cpgCO-Pen(3)—N(N(Me)2)-T-7MeW—K(NMeAc)-Pen(3)-TMAPF-2Nal-THP-K(NMeAc)—N-3Pya-Sar-CON(Me)2;
5cpaCO-Pen(3)—N(N(Me)2)-T-7MeW-Q(N(Me)2)-Pen(3)-TMAPF-2Nal-THP-Q(N(Me)2)—N-3Pya-Sar-CON(Me)2;
5cpaCO-Pen(3)—N(N(Me)2)-T-7MeW—K(NMeAc)-Pen(3)-TMAPF-2Nal-THP-K(NMeAc)—N-3Pya-Sar-CON(Me)2;
C12gEPEG2PEG2CO-hk(Me)₃-Pen(3)—N(N(Me)2)-T-7MeW—K(NMeAc)-Pen(3)-TMAPF-2Nal-THP-K(NMeAc)—N-3Pya-Sar-CON(Me)2;
C12gEPEG2PEG2CO-hk(Me)₃-Pen(3)—N(N(Me)2)-T-7MeW—K(PEG2PEG2gEC12)-Pen(3)-TMAPF-2Nal-THP-K(NMeAc)—N-3Pya-Sar-CON(Me)2;
C12gEPEG2PEG2CO-hk(Me)₃-Pen(3)—N(N(Me)2)-T-7MeW—K(NMeAc)-Pen(3)-TMAPF-2Nal-THP-K(PEG2PEG2gEC12)—N-3Pya-Sar-CON(Me)2;
C12gEPEG2PEG2CO-hk(Me)₃-Pen(3)—N(N(Me)2)-T-7MeW—K(NMeAc)-Pen(3)-TMAPF-2Nal-K(PEG2PEG2gEC12)—N-3Pya-Sar-CON(Me)2;
C12gEPEG2PEG2CO-k(PEG2PEG2gEC12)-Pen(3)—N(N(Me)2)-T-7MeW-Dab(NMeAc)-Pen(3)-TMAPF-2Nal-THP-K(NMeAc)—N-3Pya-Sar-CON(Me)2;
C14gEPEG2PEG2CO-hk(Me)₃-Pen(3)—K(PEG2PEG2gEC14)-T-7MeW—K(NMeAc)-Pen(3)-TMAPF-2Nal-THP-K(NMeAc)—N-3Pya-Sar-CON(Me)2;
C14gEPEG2PEG2CO-hk(Me)₃-Pen(3)—N(N(Me)2)-T-7MeW—K(NMeAc)-Pen(3)-TMAPF-2Nal-THP-K(NMeAc)—N-3Pya-Sar-CON(Me)2;
C14gEPEG2PEG2CO-hk(Me)₃-Pen(3)—N(N(Me)2)-T-7MeW—K(PEG2PEG2gEC14)-Pen(3)-TMAPF-2Nal-THP-K(NMeAc)—N-3Pya-Sar-CON(Me)2;
C14gEPEG2PEG2CO-hk(Me)₃-Pen(3)—N(N(Me)2)-T-7MeW—K(NMeAc)-Pen(3)-TMAPF-2Nal-THP-K(PEG2PEG2gEC14)—N-3Pya-Sar-CON(Me)2;
C14gEPEG2PEG2CO-k(PEG2PEG2gEC14)-Pen(3)—N(N(Me)2)-T-7MeW-Dab(NMeAc)-Pen(3)-TMAPF-2Nal-THP-K(NMeAc)—N-3Pya-Sar-CON(Me)2;
cPEG2gCO-Pen(3)—N(N(Me)2)-T-7MeW—K(NMeAc)-Pen(3)-TMAPF-2Nal-THP-K(NMeAc)—N-3Pya-Sar-CON(Me)2;
cPEG3aCO-Pen(3)—K(cPEG3a)-T-7MeW—K(NMeAc)-Pen(3)-TMAPF-2Nal-THP-K(NMeAc)—N-3Pya-Sar-CON(Me)2;
cPEG3aCO-Pen(3)—K(Me)₃-T-7MeW—K(NMeAc)-Pen(3)-TMAPF-2Nal-THP-K(NMeAc)—N-3Pya-Sar-CON(Me)2;
cPEG3aCO-Pen(3)—K(Me)₃-T-7MeW—K(NMeAc)-Pen(3)-TMAPF-2Nal-Achx-K(NMeAc)—N-3Pya-Sar-CON(Me)2;
cPEG3aCO-k(PEG2PEG2gEC12)-Pen(3)—K(PEG2PEG2gEC12)-T-7MeW—K(NMeAc)-Pen(3)-TMAPF-2Nal-THP-K(NMeAc)—N-3Pya-Sar-CON(Me)2;
cPEG3aCO-Pen(3)—K(PEG2PEG2gEC12)-T-7MeW—K(NMeAc)-Pen(3)-TMAPF-2Nal-THP-K(NMeAc)—N-3Pya-Sar-CON(Me)2;
cPEG3aCO-Pen(3)—K(PEG2PEG2gEC12)-T-7MeW—K(PEG2PEG2gEC12)-Pen(3)-TMAPF-2Nal-THP-K(NMeAc)—N-3Pya-Sar-CON(Me)2;
cPEG3aCO-Pen(3)—K(PEG2PEG2gEC12)-T-7MeW—K(NMeAc)-Pen(3)-TMAPF-2Nal-THP-K(PEG2PEG2gEC12)—N-3Pya-Sar-CON(Me)2;
cPEG3aCO-k(PEG2PEG2gEC14)-Pen(3)—K(PEG2PEG2gEC14)-T-7MeW—K(NMeAc)-Pen(3)-TMAPF-2Nal-THP-K(NMeAc)—N-3Pya-Sar-CON(Me)2;
cPEG3aCO-Pen(3)—K(PEG2PEG2gEC14)-T-7MeW—K(NMeAc)-Pen(3)-TMAPF-2Nal-THP-K(NMeAc)—N-3Pya-Sar-CON(Me)2;
cPEG3aCO-Pen(3)—K(PEG2PEG2gEC14)-T-7MeW—K(PEG2PEG2gEC14)-Pen(3)-TMAPF-2Nal-THP-K(NMeAc)—N-3Pya-Sar-CON(Me)2;
cPEG3aCO-Pen(3)—K(PEG2PEG2gEC14)-T-7MeW—K(NMeAc)-Pen(3)-TMAPF-2Nal-THP-K(PEG2PEG2gEC14)—N-3Pya-Sar-CON(Me)2;
cPEG3aCO-Pen(3)—N-T-7MeW—K(NMeAc)-Pen(3)-TMAPF-2Nal-THP-K(NMeAc)—N-3Pya-Sar-CONH2;
cPEG3aCO-Pen(3)—N-T-7MeW—K(NMeAc)-Pen(3)-TMAPF-2Nal-THP-K(NMeAc)—N-3Pya-Sar-CON(Me)2;
cPEG3aCO-Pen(3)—N-T-7MeW—K(NMeAC)-Pen(3)-TMAPF-2Nal-THP-Dab(NMeAc)—N-3Pya-Sar-CON(Me)2;
cPEG3aCO-Pen(3)—N-T-7MeW-Dab(NMeAc)-Pen(3)-TMAPF-2Nal-THP-K(NMeAC)—N-3Pya-Sar-CON(Me)2;
cPEG3aCO-Pen(3)—N-T-7MeW—K(Ac)-Pen(3)-TMAPF-2Nal-THP-A-N-3Pya-Sar-CON(Me)2;
cPEG3aCO-Pen(3)—N-T-7MeW—K(Ac)-Pen(3)-TMAPF-2Nal-THP-E-N-3Pya-Sar-CONH2;
cPEG3aCO-Pen(3)—N-T-7MeW—K(Ac)-Pen(3)-TMAPF-2Nal-THP—K(Ac)—N-3Pya-Sar-CONH2;
cPEG3aCO-Pen(3)—N-T-7(3NAcPh)W—K(Ac)-Pen(3)-TMAPF-2Nal-THP-E-N-3Pya-Sar-CONH2;
cPEG3aCO-Pen(3)—N(N(Me))-T-7MeW—K(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-THP-E-N-3Pya-Sar-CON(Me)2;
cPEG3aCO-Pen(3)—N(N(Me)2)-T-7MeW—K(NMeAc)-Pen(3)-TMAPF-2Nal-THP-K(NMeAc)—N-3Pya-Sar-CO(NHPEG3a);
cPEG3aCO-Pen(3)—N(N(Me)2)-T-7MeW-APEG2Ser(S*)-Pen(3)-TMAPF-2Nal-THP-K(NMeAc)—N-3Pya-Sar-CON(Me)2;
cPEG3aCO-Pen(3)—N(N(Me)2)-T-7MeW-APEG2Ser(R*)-Pen(3)-TMAPF-2Nal-THP-K(NMeAc)—N-3Pya-Sar-CON(Me)2;
cPEG3aCO-Pen(3)—N(N(Me)2)-T-7(3NAcPh)W—K(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(NMeAC)-Pen(3)-TMAPF-2Nal-THP-Dab(NMeAc)—N-3Pya-Sar-CON(Me)2;
cPEG3aCO-Pen(3)—N(N(Me)2)-T-7MeW—K(Ac)-Pen(3)-TMAPF-2Nal-THP-Dab(NMeAc)—N-3Pya-Sar-CON(Me)2;
cPEG3aCO-Pen(3)—N(N(Me)2)-T-7MeW—K(NMeAC)-Pen(3)-TMAPF-2Nal-THP-Q(N(Me)2)—N-3Pya-Sar-CON(Me)2;
cPEG3aCO-Pen(3)—N(N(Me)2)-T-7MeW-Q(N(Me)2)-Pen(3)-TMAPF-2Nal-THP-K(NMeAC)—N-3Pya-Sar-CON(Me)2;
cPEG3aCO-Pen(3)—N(N(Me)2)-T-7MeW—K(NMeAc)-Pen(3)-TMAPF-2Nal-THP-A-N-3Pya-Sar-CON(Me)2;
cPEG3aCO-Pen(3)—N(N(Me)2)-T-7MeW—K(NMeAc)-Pen(3)-TMAPF-2Nal-THP-K(NMeAc)—N-3Pya-Sar-CONH2;
cPEG3aCO-Pen(3)—N(N(Me)2)-T-7MeW—K(NMeAc)-Pen(3)-TMAPF-2Nal-THP-Cit-N-3Pya-Sar-CON(Me)2;
cPEG3aCO-Pen(3)—N(N(Me)2)-T-7MeW—K(Me)₃-Pen(3)-TMAPF-2Nal-THP-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-Acpx-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-Achx-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-Chg-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-Aib-K(NMeAc)—N-3Pya-Sar-CON(Me)2;
cPEG3aCO-Pen(3)—N(N(Me)2)-T-7MeW-Cit-Pen(3)-TMAPF-2Nal-THP-K(NMeAc)—N-3Pya-Sar-CON(Me)2;
cPEG3aCO-Pen(3)—N(N(Me)2)-T-7MeW-Dab(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW-Dab(NMeAc)-Pen(3)-TMAPF-2Nal-THP-K(NMeAc)—N-3Pya-Sar-CONH2;
cPEG3aCO-k(PEG2PEG2gEC12)-Pen(3)—N(N(Me)2)-T-7MeW—K(NMeAc)-Pen(3)-TMAPF-2Nal-THP-K(NMeAc)—N-3Pya-Sar-CON(Me)2;
cPEG3aCO-k(PEG2PEG2gEC14)-Pen(3)—N(N(Me)2)-T-7MeW—K(NMeAc)-Pen(3)-TMAPF-2Nal-THP-K(NMeAc)—N-3Pya-Sar-CON(Me)2;
cPEG3aCO-k(PEG2PEG2gEC12)-Pen(3)—N(N(Me)2)-T-7MeW—K(PEG2PEG2gEC12)-Pen(3)-TMAPF-2Nal-THP-K(NMeAc)—N-3Pya-Sar-CON(Me)2;
cPEG3aCO-Pen(3)—N(N(Me)2)-T-7MeW—K(cPEG3a)-Pen(3)-TMAPF-2Nal-THP-K(NMeAc)—N-3Pya-Sar-CON(Me)2;
cPEG3aCO-Pen(3)—N(N(Me)2)-T-7MeW—K(4cpg)-Pen(3)-TMAPF-2Nal-THP-K(NMeAc)—N-3Pya-Sar-CON(Me)2;
cPEG3aCO-k(PEG2PEG2gEC12)-Pen(3)—N(N(Me)2)-T-7MeW—K(NMeAc)-Pen(3)-TMAPF-2Nal-THP-K(PEG2PEG2gEC12)—N-3Pya-Sar-CON(Me)2;
cPEG3aCO-k(PEG2PEG2gEC14)-Pen(3)—N(N(Me)2)-T-7MeW—K(NMeAc)-Pen(3)-TMAPF-2Nal-THP-K(PEG2PEG2gEC14)—N-3Pya-Sar-CON(Me)2;
cPEG3aCO-Pen(3)—N(N(Me)2)-T-7MeW—K(PEG2PEG2gEC14)-Pen(3)-TMAPF-2Nal-THP-K(NMeAc)—N-3Pya-Sar-CON(Me)2;
cPEG3aCO-Pen(3)—N(N(Me)2)-T-7MeW—K(PEG2PEG2gEC12)-Pen(3)-TMAPF-2Nal-THP-K(PEG2PEG2gEC12)—N-3Pya-Sar-CON(Me)2;
cPEG3aCO-Pen(3)—N(N(Me)2)-T-7MeW—K(NMeAc)-Pen(3)-TMAPF-2Nal-THP-K(PEG2PEG2gEC12)—N-3Pya-Sar-CON(Me)2;
cPEG3aCO-Pen(3)—N(N(Me)2)-T-7MeW—K(NMeAc)-Pen(3)-TMAPF-2Nal-THP-K(PEG2PEG2gEC14)—N-3Pya-Sar-CON(Me)2;
cPEG3aCO-Pen(3)—N(N(Me)2)-T-7MeW—K(PEG2PEG2gEC14)-Pen(3)-TMAPF-2Nal-THP-K(PEG2PEG2gEC14)—N-3Pya-Sar-CON(Me)2;
cPEG3aCO-Pen(3)—N(N(Me)2)-T-7MeW-Dap(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(Ac)-Pen(3)-TMAPF-2Nal-THP-Dap(NMeAc)—N-3Pya-Sar-CON(Me)2;
cPEG3gCO-Pen(3)—N(N(Me)2)-T-7MeW—K(NMeAc)-Pen(3)-TMAPF-2Nal-THP-K(NMeAc)—N-3Pya-Sar-CON(Me)2;
MeCO-Pen(3)—N-T-7MeW—K(NMeAc)-Pen(3)-TMAPF-2Nal-THP-K(NMeAc)—N-3Pya-Sar-CONH2;
MeCO-Pen(3)—N-T-7MeW—K(Ac)-Pen(3)-TMAPF-2Nal-THP—K(Ac)—N-3Pya-Sar-CONH2;
MeCO-Pen(3)—N-T-7MeW—K(Ac)-Pen(3)-TMAPF-2Nal-THP-E-N-3Pya-Sar-CONH2;
MeCO-k(Me)₃-Pen(3)—N-T-7MeW—K(Ac)-Pen(3)-TMAPF-2Nal-THP-E-N-3Pya-Sar-CONH2;
MeCO-k(Me)₃-Pen(3)—N-T-7(3NAcPh)W—K(Ac)-Pen(3)-TMAPF-2Nal-THP-E-N-3Pya-Sar-CONH2;
MeCO-k(Me)₃-Pen(3)—N-T-7MeW—K(Ac)-Pen(3)-TMAPF-2Nal-THP—K(Ac)—N-3Pya-Sar-CONH2;
MeCO-Pen(3)—N-T-7(3NAcPh)W—K(Ac)-Pen(3)-TMAPF-2Nal-THP-E-N-3Pya-Sar-CONH2;
MeCO-Pen(3)—N-T-7(3NAcPh)W—K(Ac)-Pen(3)-TMAPF-2Nal-THP—K(Ac)—N-3Pya-Sar-CONH2;
MeCO-r-Pen(3)—N-T-7MeW—K(Ac)-Pen(3)-TMAPF-2Nal-THP-E-N-3Pya-Sar-CONH2;
MeCO-Pen(3)—N(N(Me)2)-T-7MeW—K(NMeAc)-Pen(3)-TMAPF-2Nal-THP-K(NMeAc)—N-3Pya-Sar-CON(Me)2;
MeCO-Pen(3)—N(N(Me)2)-T-7MeW—K(NMeAc)-Pen(3)-TMAPF-2Nal-THP-K(NMeAc)—N-3Pya-Sar-CO(NHPEG3a);
MeCO-Pen(3)—N(N(Me)2)-T-7MeW—K(NMeAc)-Pen(3)-TMAPF-2Nal-THP-APEG2Ser-N-3Pya-Sar-CON(Me)2;
MeCO-hk(Me)₃-Pen(3)—N(N(Me)2)-T-7MeW—K(NMeAc)-Pen(3)-TMAPF-2Nal-THP-K(NMeAc)—N-3Pya-Sar-CON(Me)2;
MeCO-r-Pen(3)—N(N(Me)2)-T-7MeW—K(NMeAc)-Pen(3)-TMAPF-2Nal-THP-K(NMeAc)—N-3Pya-Sar-CON(Me)2; or a pharmaceutically acceptable salt thereof.

In another aspect of the invention:

19. A compound which is:

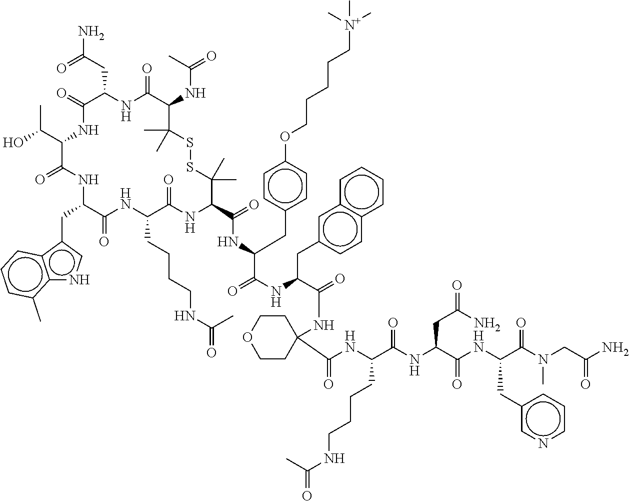

or a pharmaceutically acceptable salt thereof.

In another aspect of the invention:

20. A compound which is

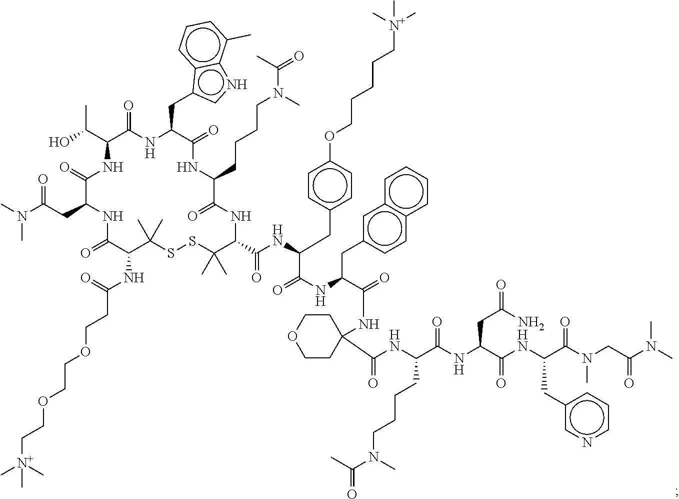

pharmaceutically acceptable salt thereof.

In another aspect of the invention:

21. The compound or pharmaceutically acceptable salt thereof, according to any one of aspects 19 and 20 wherein the quarternary ammonium salts as shown in the chemical structures therein include a counterion AN⁻selected from: acetate, adipate, benzoate, benzenesulfonate, citrate, decanoate, chloride, lactate, maleate, methanesulfonate, oxalate, pivalate, propionate, succinate, sulfate, tartrate, or trifluoroacetate.

In another aspect of the invention:

22. A pharmaceutical composition comprising the peptide inhibitor or pharmaceutically acceptable salt thereof of any one of aspect 1-21, and a pharmaceutically acceptable carrier, excipient, or diluent.

In another aspect of the invention:

23. The pharmaceutical composition of aspect 22, further comprising an enteric coating.

In another aspect of the invention:

24. The pharmaceutical composition of aspect 22, wherein the enteric coating protects and releases the pharmaceutical composition within a subject's lower gastrointestinal system.

In another aspect of the invention:

24. A method for treating an Inflammatory Bowel Disease (IBD), ulcerative colitis, Crohn's disease, Celiac disease (nontropicalSprue), enteropathy associated with seronegative arthropathies, microscopic colitis, collagenous colitis, eosinophilic gastroenteritis, colitis associated with radio- or chemo-therapy, colitis associated with disorders of innate immunity as in leukocyte adhesion deficiency-1, chronic granulomatous disease, glycogen storage disease type 1b, Hermansky-Pudlak syndrome, Chediak-Higashi syndrome, and Wiskott-Aldrich Syndrome, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, psoriasis, psoriatic arthritis, or graft versus host disease in a subject, comprising providing to the subject an effective amount of the peptide inhibitor or peptide inhibitor or pharmaceutically acceptable salt thereof of any one of aspects 1-21, or the pharmaceutical composition of any one of aspects 22-24.

In another aspect of the invention:

26. The method of aspect 25, wherein the pharmaceutical composition is provided to the subject by an oral, parenteral, intravenous, peritoneal, intradermal, subcutaneous, intramuscular, intrathecal, inhalation, vaporization, nebulization, sublingual, buccal, parenteral, rectal, intraocular, inhalation, topically, vaginal, or topical route of administration.

In another aspect of the invention:

27. The method of aspect 24 for treating Inflammatory Bowel Disease (IBD), ulcerative colitis, or Crohn's disease, wherein the pharmaceutical composition is provided to the subject orally.

In another aspect of the invention:

28. The method of aspect 24 for treating psoriasis, wherein the pharmaceutical composition is provided to the subject orally, topically, parenterally, intravenously, subcutaneously, peritonealy, or intravenously.

In another aspect of the invention:

29. The peptide inhibitor or peptide inhibitor or pharmaceutically acceptable salt thereof of any one of aspects 1-21, or the pharmaceutical composition of any one of aspects 22-24, for use in the treatment of an Inflammatory Bowel Disease (IBD), ulcerative colitis, Crohn's disease, Celiac disease (nontropicalSprue), enteropathy associated with seronegative arthropathies, microscopic colitis, collagenous colitis, eosinophilic gastroenteritis, colitis associated with radio- or chemo-therapy, colitis associated with disorders of innate immunity as in leukocyte adhesion deficiency-1, chronic granulomatous disease, glycogen storage disease type 1b, Hermansky-Pudlak syndrome, Chediak-Higashi syndrome, and Wiskott-Aldrich Syndrome, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, psoriasis, psoriatic arthritis, or graft versus host disease in a subject, comprising providing to the subject an effective amount of the peptide inhibitor or peptide inhibitor or pharmaceutically acceptable salt thereof of any one of aspects 1-21, or the pharmaceutical composition of any one of aspects 22-24.

In another aspect of the invention:

30. The composition for use according to aspect 29, wherein the pharmaceutical composition is provided to the subject by an oral, parenteral, intravenous, peritoneal, intradermal, subcutaneous, intramuscular, intrathecal, inhalation, vaporization, nebulization, sublingual, buccal, parenteral, rectal, intraocular, inhalation, topically, vaginal, or topical route of administration.

In another aspect of the invention:

31. The composition for use according to aspect 29 for treating Inflammatory Bowel Disease (IBD), ulcerative colitis, or Crohn's disease, wherein the pharmaceutical composition is provided to the subject orally.

In another aspect of the invention:

32. The composition for use according to aspect 29 for treating psoriasis, wherein the pharmaceutical composition is provided to the subject orally, topically, parenterally, intravenously, subcutaneously, peritonealy, or intravenously.

In another aspect of the invention:

33. A compound which is selected from of any of the tables 1A, 1B, 1C, 1D, and lE or pharmaceutically acceptable salts thereof.

In another aspect of the invention:

34. A compound of Formula (I) or (II) wherein X3 is

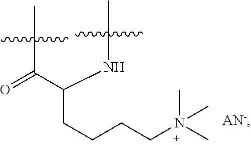

or a pharmaceutically acceptable salt thereof

35. A compound of Formula (VII)

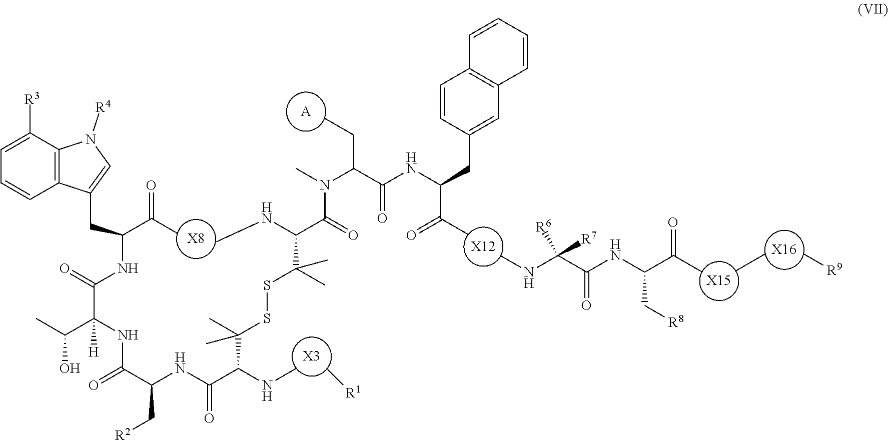

wherein:
A is

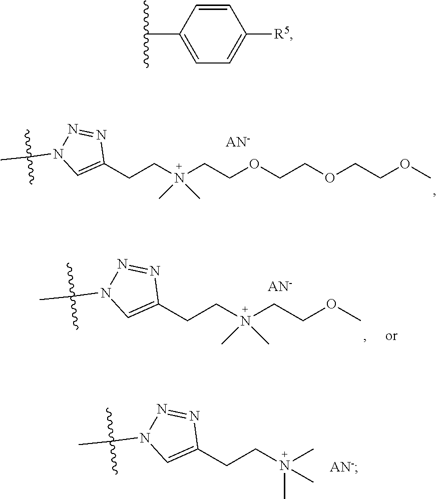

wherein:
   R⁵ is —OCHF₂, —O(CH₂)₉CO₂H,

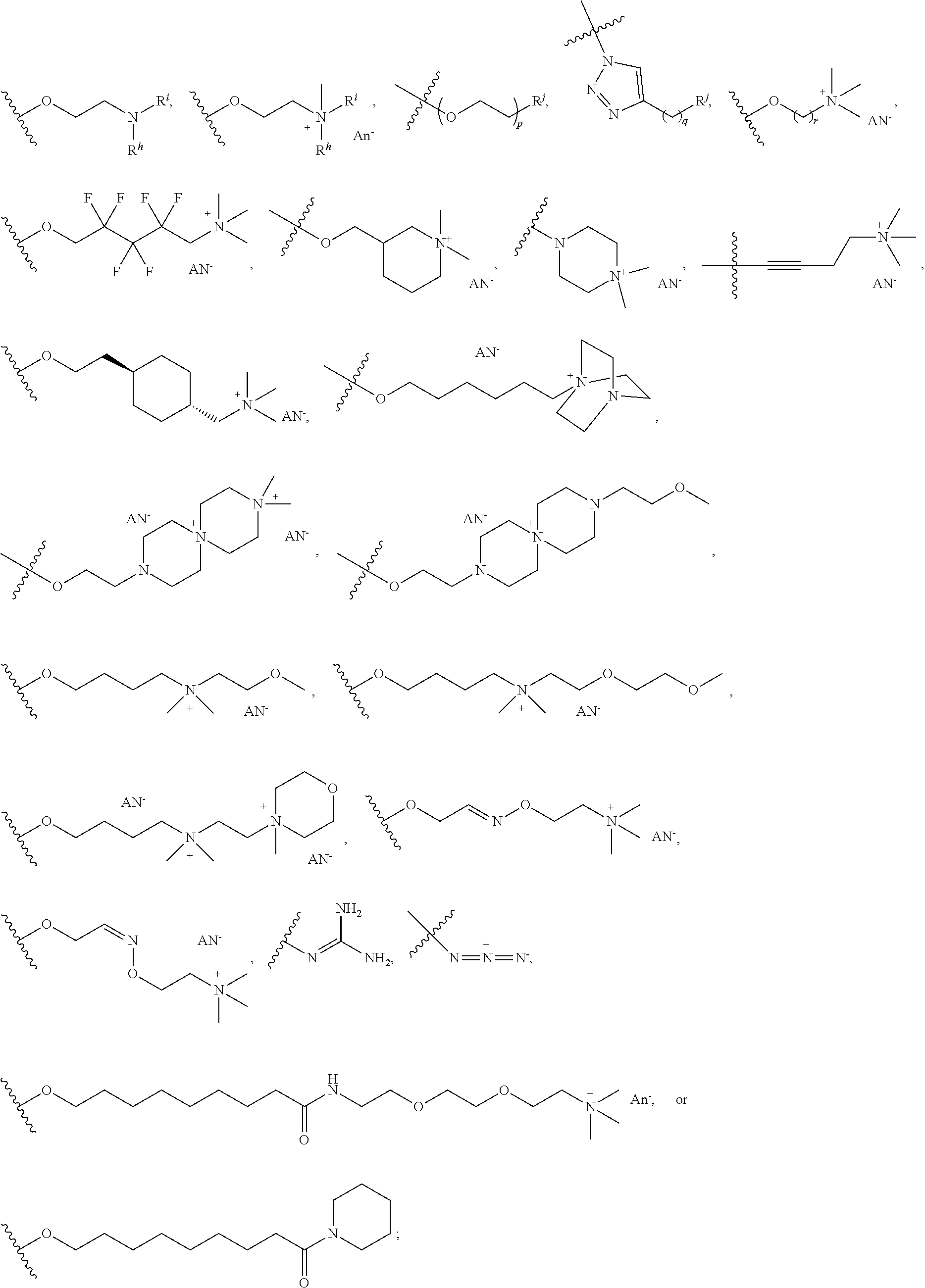

wherein:
   R^{h} is H, —CH₃, —C(O)CH₃, —C(NH)NH₂, —(CH₂)₃O(CH₂)₂OCH₃, —CH₂CH₂OCH₃, —(CH₂CH₂O)₃CH₃, —(CH₂CH₂O)₆CH₃,

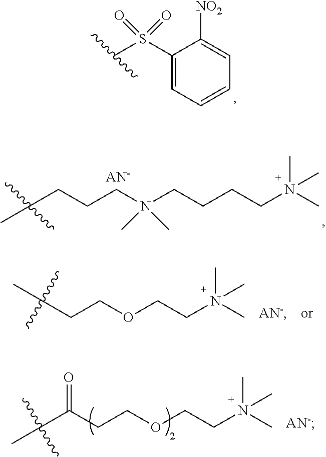

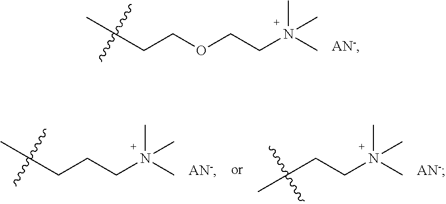

Rⁱ is H, —CH₃, —(CH₂)₃O(CH₂)₂OCH₃,
p is 1, 2 or 3;
R^{j} is

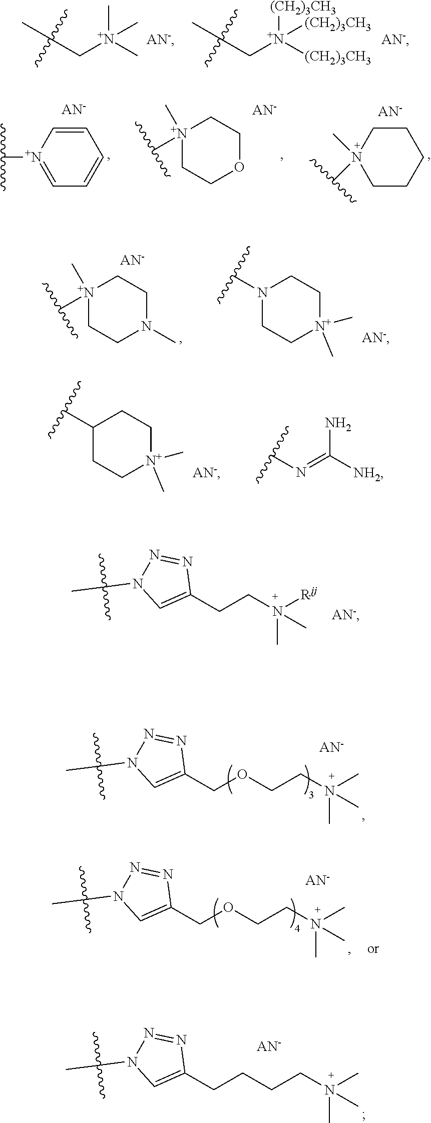

q is 1, 2, 3, 4, or 5;
   R^{k} is —OCH₃,

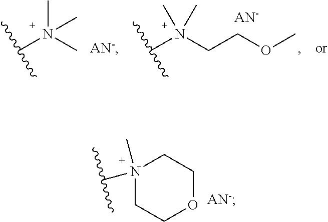

r is 3, 4, 5, 6, or 8;
wherein:
   R^{jj} is H, —(CH₂)₇CH₃, —(CH₂)₁₅CH₃, —(CH₂)₂OCH₃, or —(CH₂CH₂O)₃CH₃;
R¹ is —C(O)C(1-2)alkyl, —C(O)CF₃, —COCH₂CF₃, —CO(CH₂)₅N(CH₃)₃⁺AN⁻, —CH₂CH₂NHC(O)CH₂O(CH₂CH₂O)₂CH₃, —CH₂CH₂NHC(O)(CH₂CH₂O)₆CH₃,

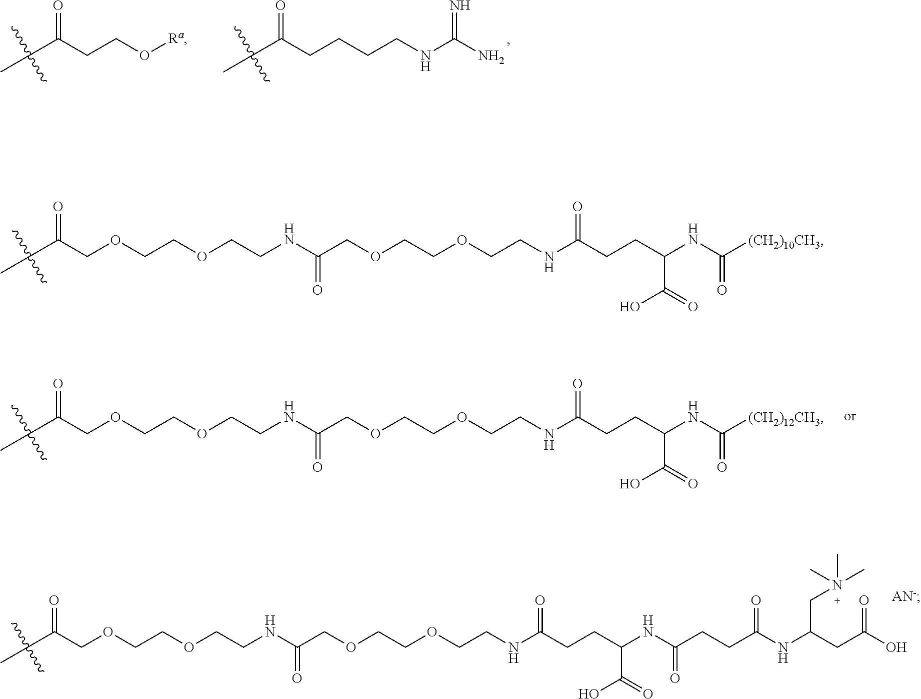

wherein:
   R^{a} is

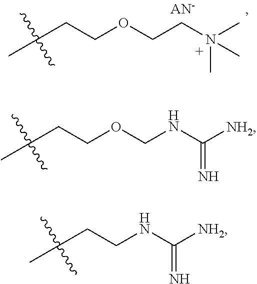

CH₂CH₂OCH₂CH₂N(CH₃)₂,
—(CH₂CH₂O)₃CH₂CH₂N(CH₃)₂, —(CH₂CH₂O)₃CH₂CH₂N(CH₃)₃ ⁺AN⁻, —(CH₂CH₂O)₅CH₂CH₂NC(O)CH₃, —(CH₂CH₂O)₈CH₂CH₂NC(O)CH₃, or —(CH₂CH₂O)₁₁CH₂CH₂NC(O)CH₃;
X3 is a direct bond, (D) arginine, (D) lysine substituted with R^{b},

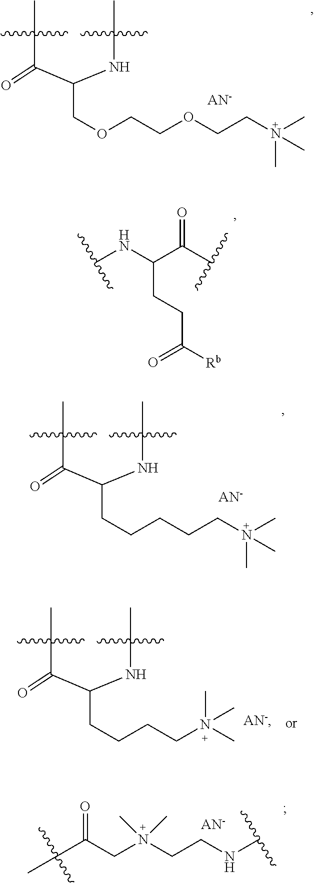

wherein:
   R^{b} is

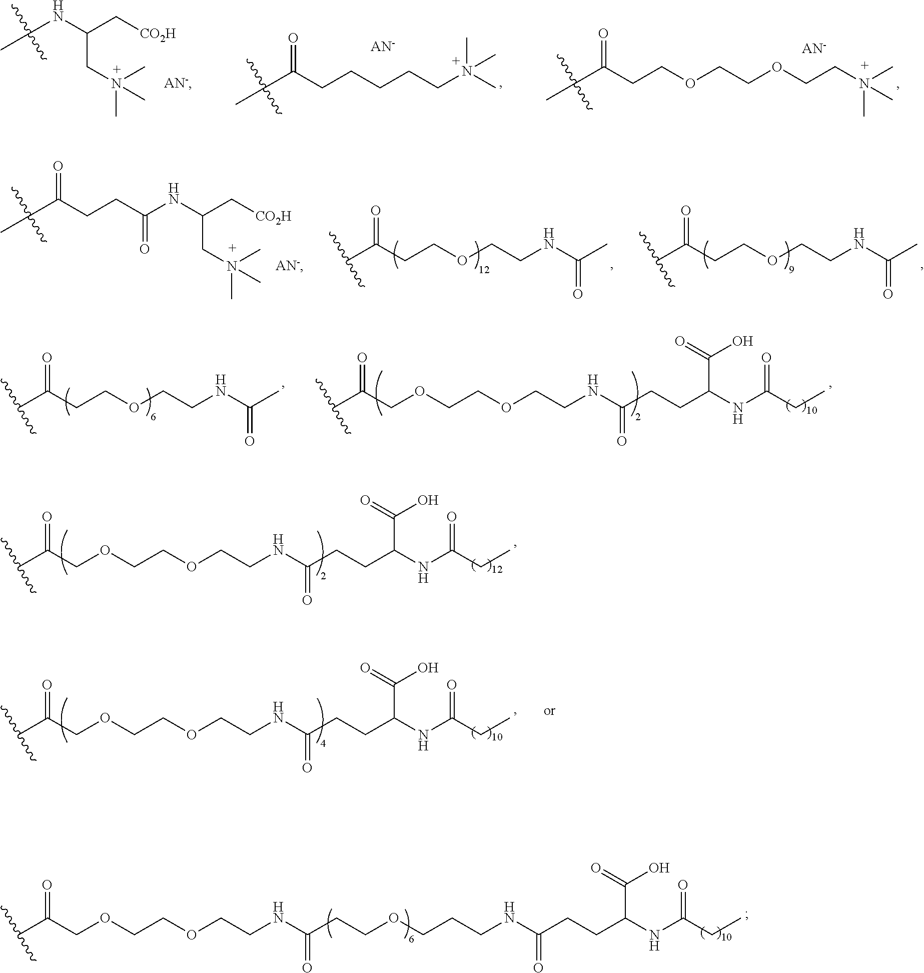

R² is H, —CH(CH₃)₂,

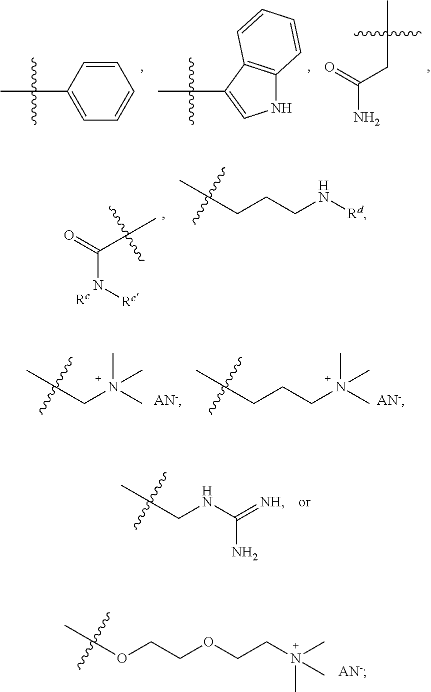

wherein:
   R^{c} is H, or —CH₃;
   R^{c}′ is H, or —CH₃;
   R^{d} is

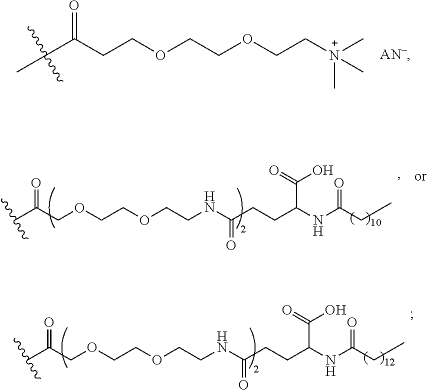

R³ is H, —CH₃, —CF₃, or

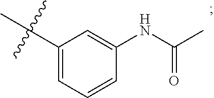 [SMILES: *c1cccc(NC(C)=O)c1]

R⁴ is H, or —CH₃;
X8 is

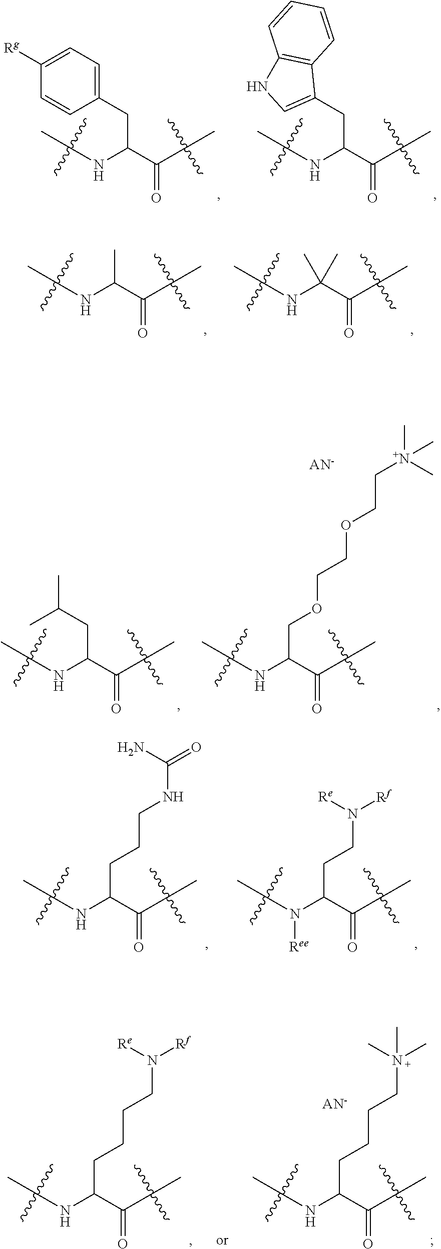

wherein:
   R^{e} is H, or —CH₃;
   R^{ee} is H;
   R^{f} is H, —CH₃, —C(CH₃)₃, —C(O)CH₃, —C(O)(CH₂CH₂O)₆CH₃, —(CH₂CH₂O)₆CH₃,

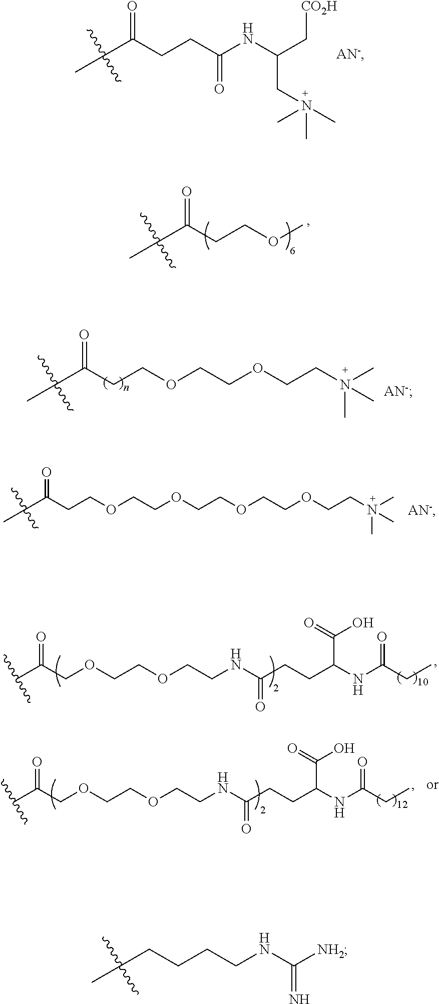

R^{g} is H, —OH, —C(O)NH₁₂, or —CH₂NHC(O)CH₃;
n is 0, or 1;
X12 is absent,

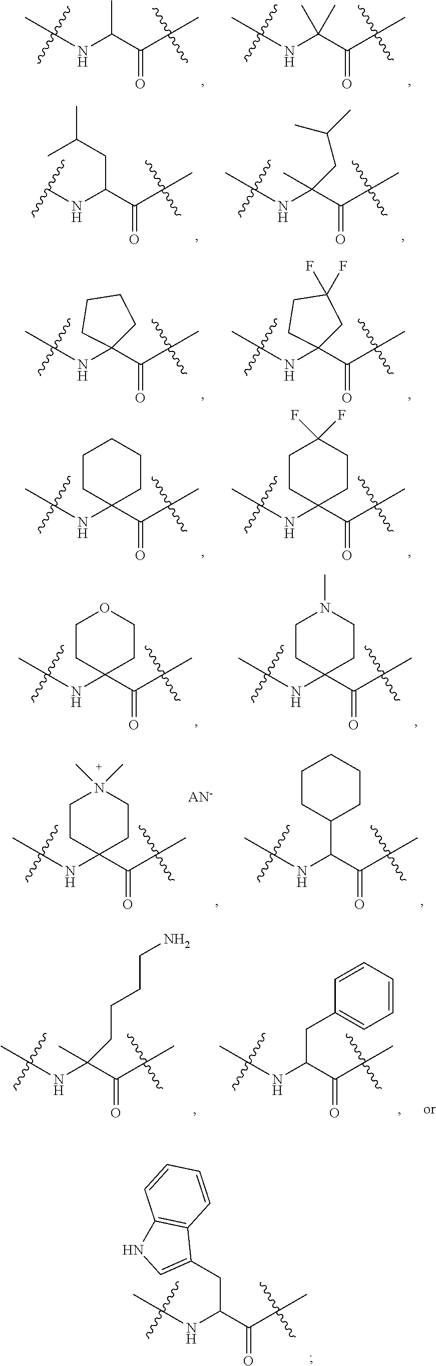

R⁶ is —CH₃, —CH₂CH₂C(O)R^{m}, ⁿ(CH₂)ₛNRⁿR^{o},

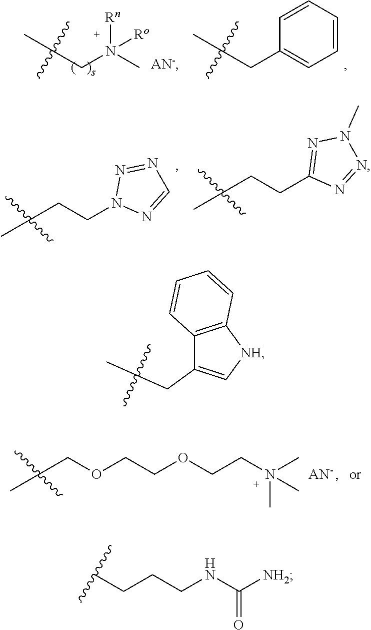

R⁷ is H, or —CH3;
   wherein:
      R^{m} is —OH, or

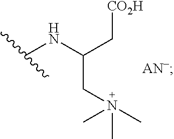

Rⁿ is H, —CH₃, —CH₂CH(CH₃)₂, —C(O)CH₃, —C(O)(CH₂CH₂O)₆CH₃, —(CH₂CH₂O)₆CH₃, —C(O)(CH₂CH₂O)ₜCH₂CH₂R^{p},

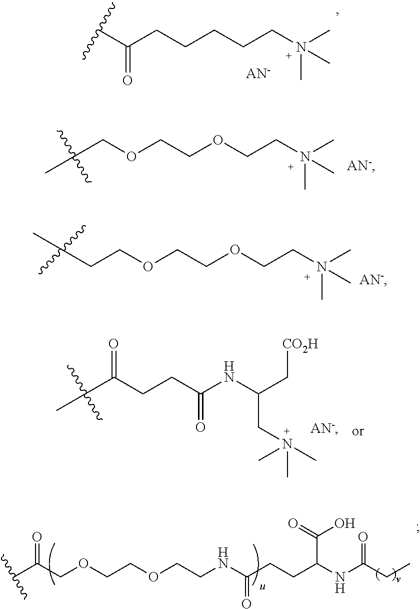

R^{o} is H, —CH₃, or —C(O)CH₃;
wherein:
   R^{p} is NHC(O)CH₃, or

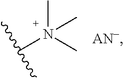

s is 1, 2, or 4;
t is 2, 6, 9, or 12;
u is 2 or 3;
v is 10 or 12;
R⁸ is —CH₂CH₂CH₂NHC(O)CH₃, —C(O)NH₂, or —C(O)NHCH₃;
X15 is absent,

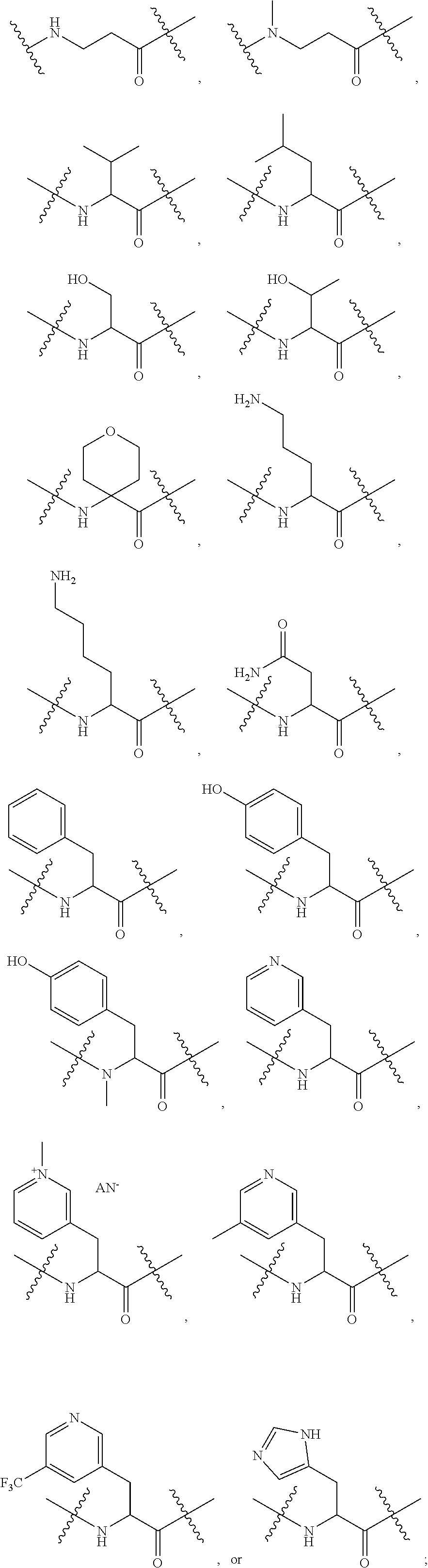

X16 is absent

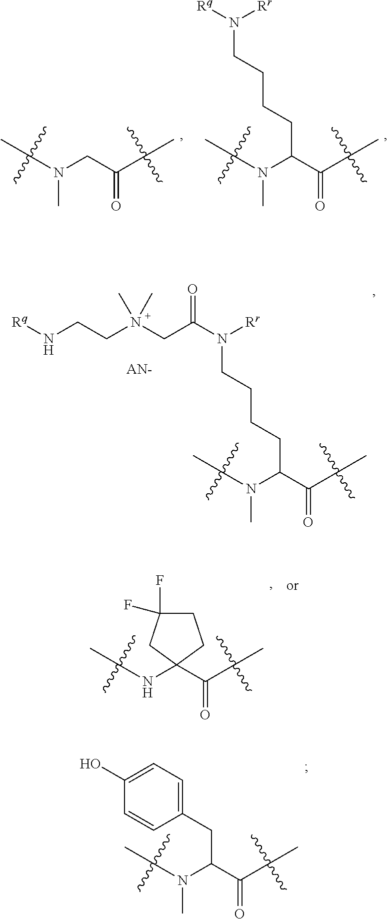

wherein:
   R^{q}

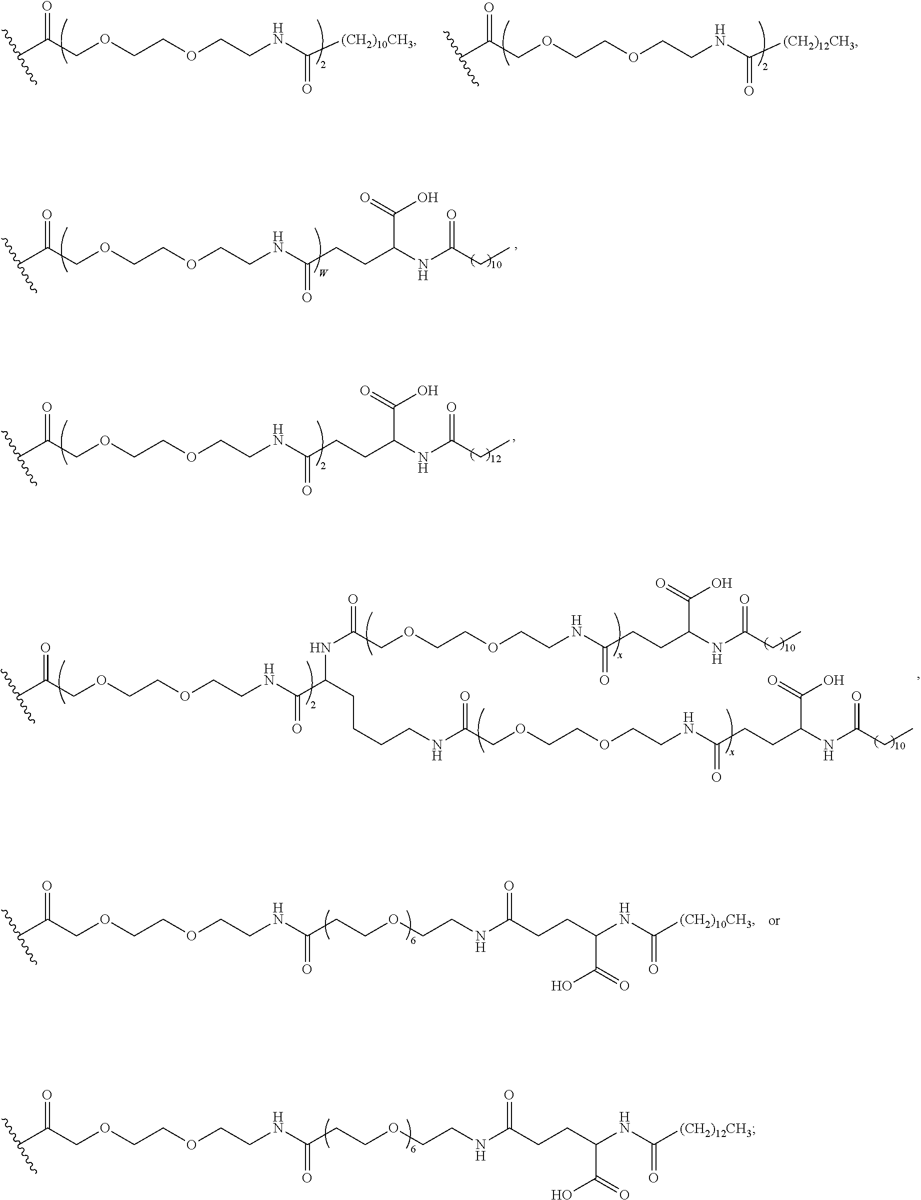

R^{r} is H;
w is 24, or 5;
x is 20r 4; and
R⁹ is —NH₂, —NHCH₃, —N(CH₃)₂, —NH(CH₂CH₂O)₂CH₃, —N(CH₃)(CH₂CH₂O)₂CH₃, —NH(CH₂CH₂O)_{y}R^{s},

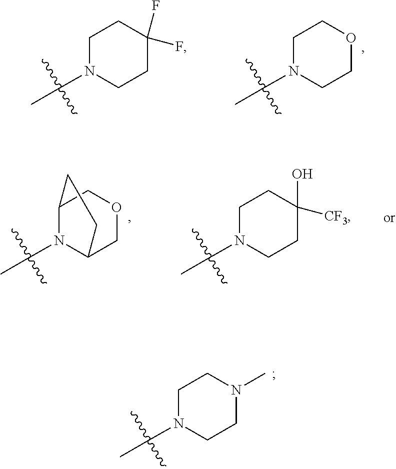

wherein:
   y is 2, 4, or 6;
   R^{s} is —CH₃ or

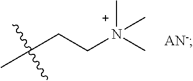 [SMILES: *CC[N+](C)(C)C]

wherein:
AN⁻as above defined for A, R, X3, X12, R1, R2, R5, R6, R8, R15, R16 and R^{a}, R^{b}, R^{d}, R^{f}, R^{h}, Rⁱ, R^{j}, R^{k}, R^{m}, Rⁿ, R^{o}, R^{s} is acetate, adipate, benzoate, benzenesulfonate, citrate, decanoate, chloride, lactate, maleate, methanesulfonate, oxalate, pivalate, propionate, succinate, sulfate, tartrate, or trifluoroacetate; or
a pharmaceutically acceptable salt thereof.

In another aspect of the invention

36. A compound of Formula (VIII)

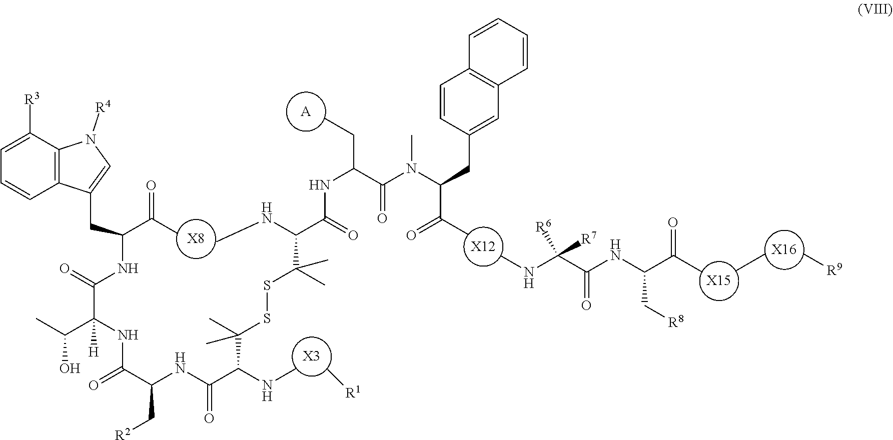

wherein:
A is

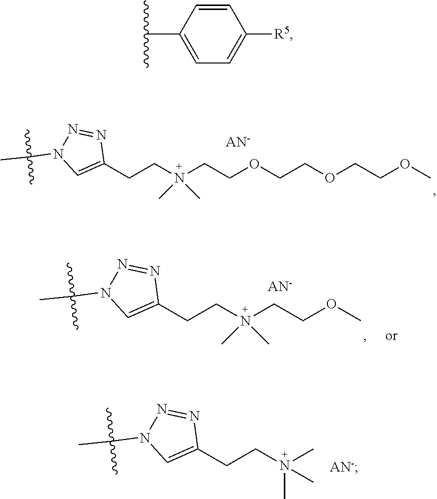

wherein:
   R⁵ is —OCHF₂, —O(CH₂)₉CO₂H,

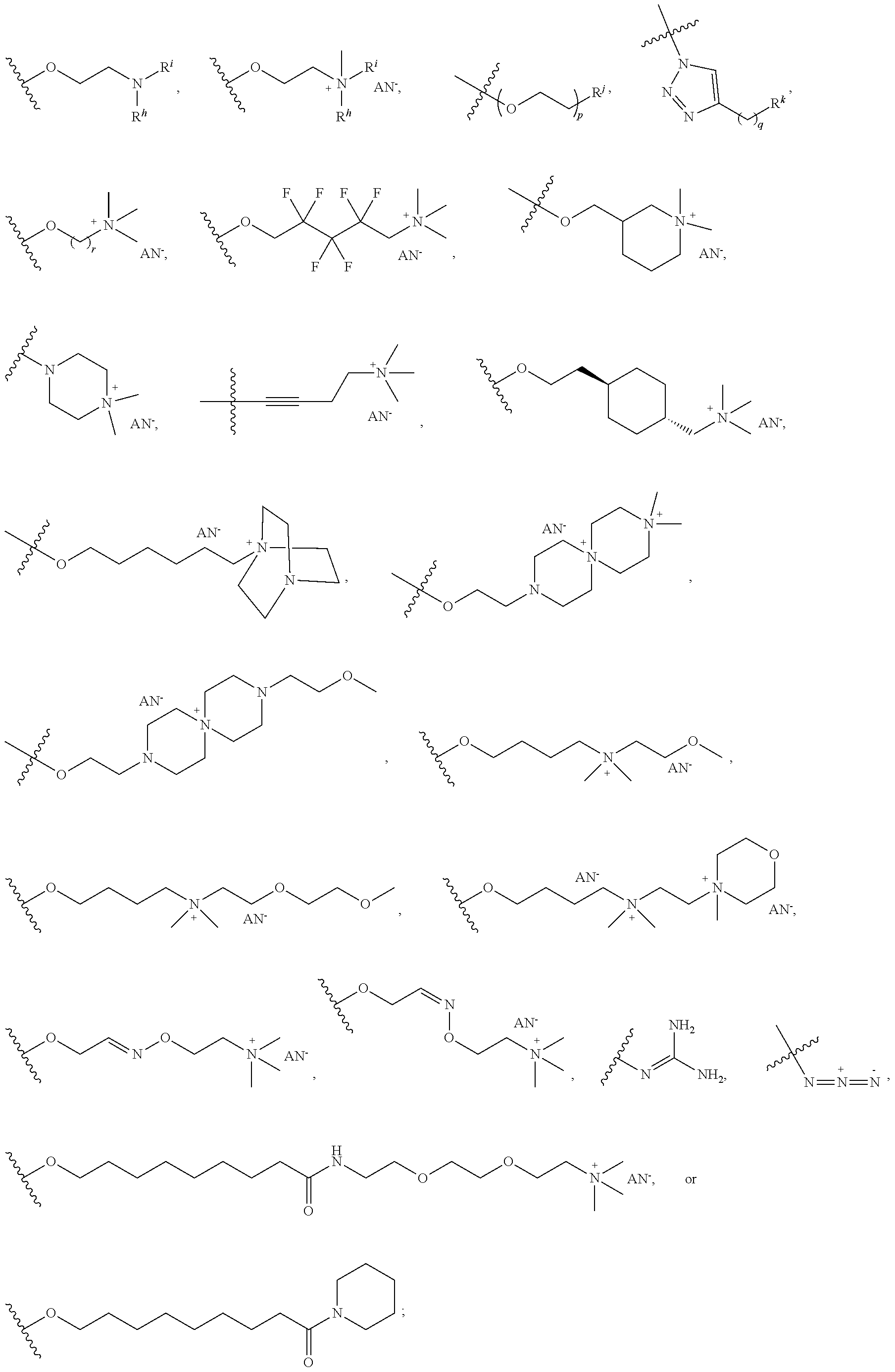

wherein:
   R^{h} is H, —CH₃, —C(O)CH₃, —C(NH)NH₂, —(CH₂)₃O(CH₂)₂OCH₃, —CH₂CH₂OCH₃, —(CH₂CH₂O)₃CH₃, —(CH₂CH₂O)₆CH₃,

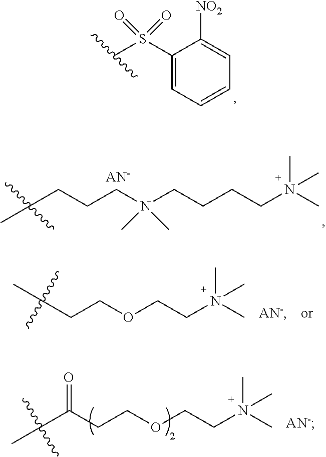

Rⁱ is H, —CH₃, —(CH₂)₃O(CH₂)₂OCH₃,

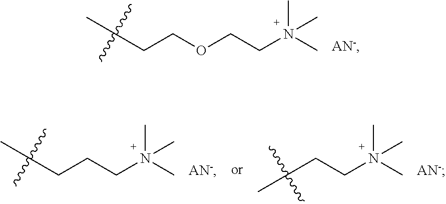

p is 1, 2 or 3;
R^{j} is

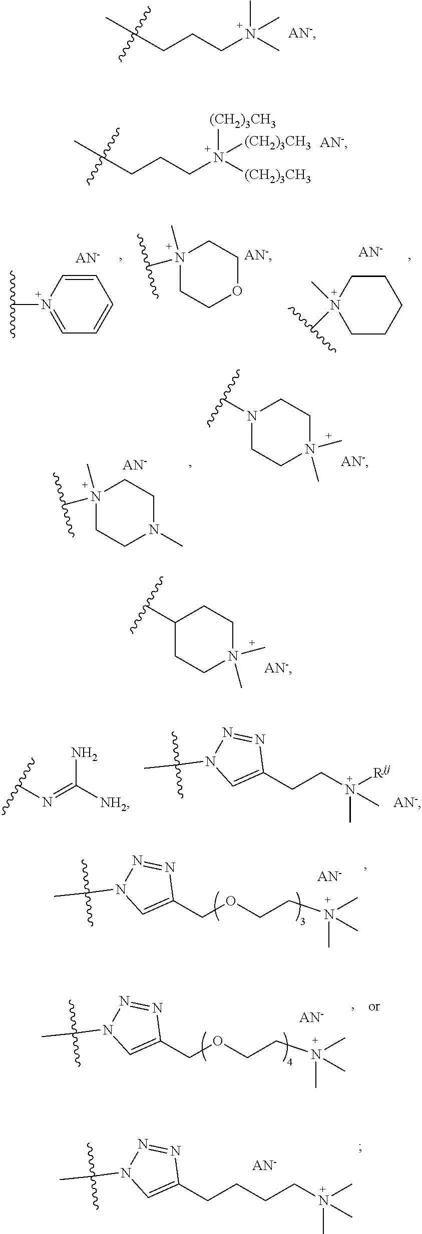

q is 1, 2, 3, 4, or 5;
R^{k} is —OCH₃,

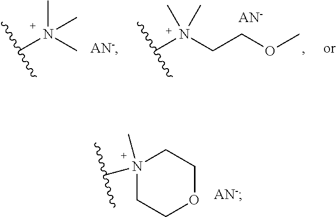

r is 3, 4, 5, 6, or 8;
wherein:
   R^{jj} is H, —(CH₂)₇CH₃, —(CH₂)₁₅CH₃, —(CH₂)₂OCH₃, or —(CH₂CH₂O)₃CH₃;
R¹ is —C(O)C(1-2)alkyl, —C(O)CF₃, —COCH₂CF₃, —CO(CH₂)₅N(CH₃)₃+AN, —CH₂CH₂NHC(O)CH₂O(CH₂CH₂O)₂CH₃, —CH₂CH₂NHC(O)(CH₂CH₂O)₆CH₃,

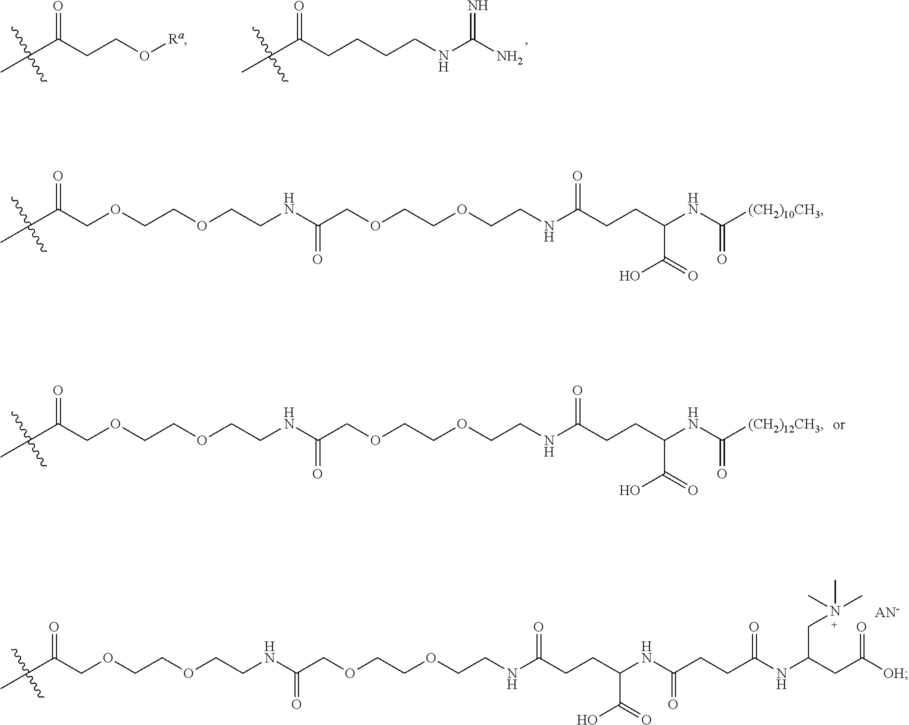

wherein
   R^{a} is,

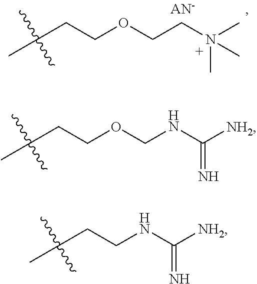

CH₂CH₂OCH₂CH₂N(CH₃)₂,
—(CH₂CH₂O)₃CH₂CH₂N(CH₃)₂, —(CH₂CH₂O)₃CH₂CH₂N(CH₃)₃ ⁺AN⁻, —(CH₂CH₂O)₅CH₂CH₂NC(O)CH₃, —(CH₂CH₂O)₈CH₂CH₂NC(O)CH₃, or —(CH₂CH₂O)₁₁CH₂CH₂NC(O)CH₃;
X3 is a direct bond, (D) arginine, (D) lysine substituted with R^{b},

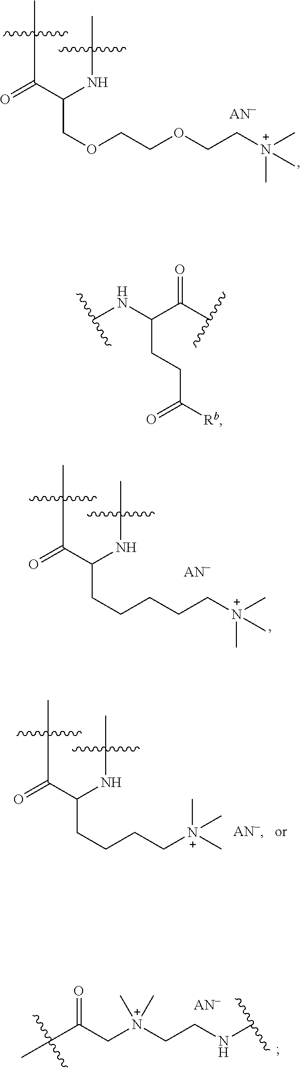

wherein:
   R^{b} is

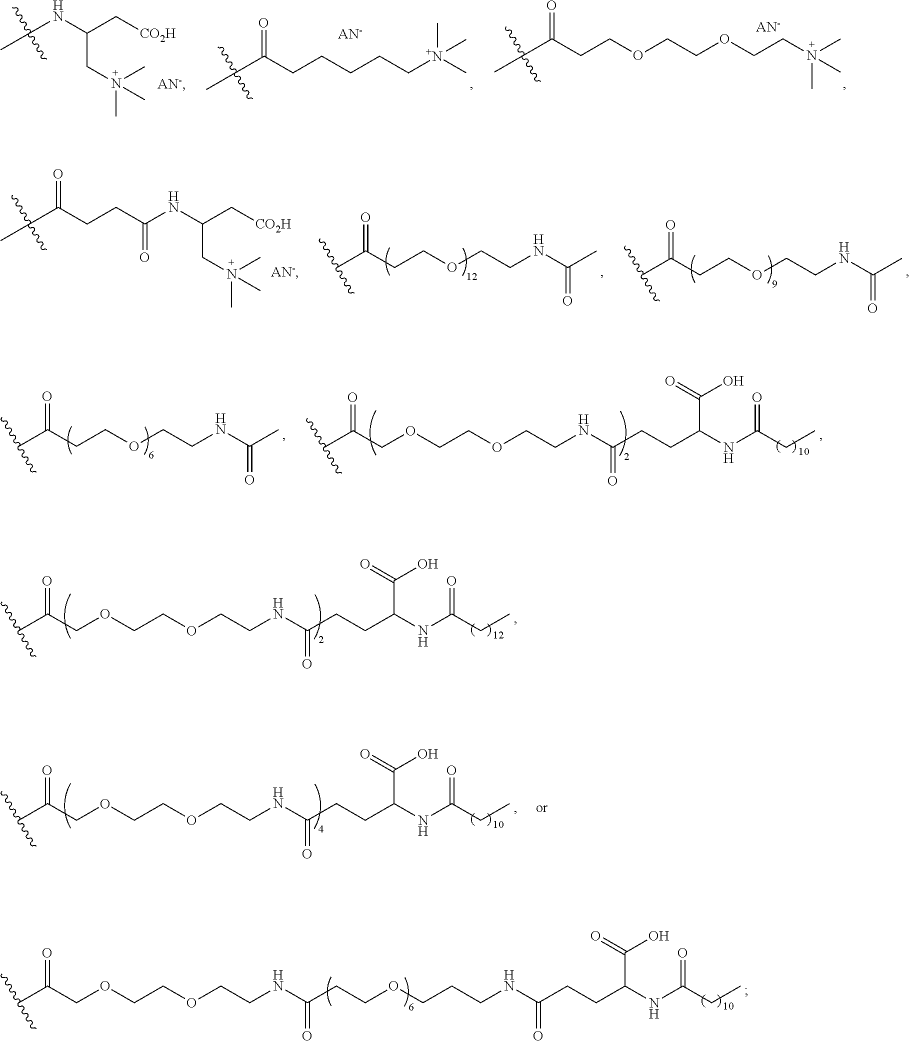

R² is H, —CH(CH₃)₂,

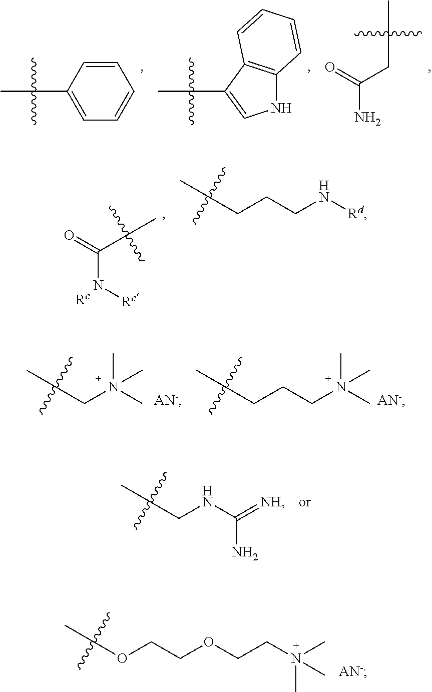

wherein:
   R^{c} is H, or —CH₃;
   R^{c}′ is H, or —CH₃;
   R^{d} is

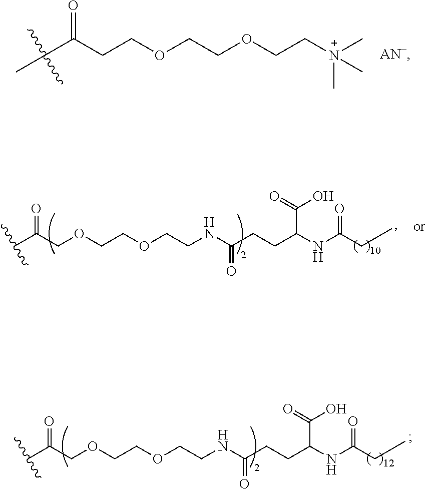

R³ is H, —CH₃, —CF₃, or

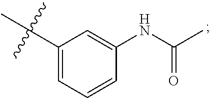 [SMILES: *c1cccc(NC(C)=O)c1]

R⁴ is H, or —CH₃;
X8 is

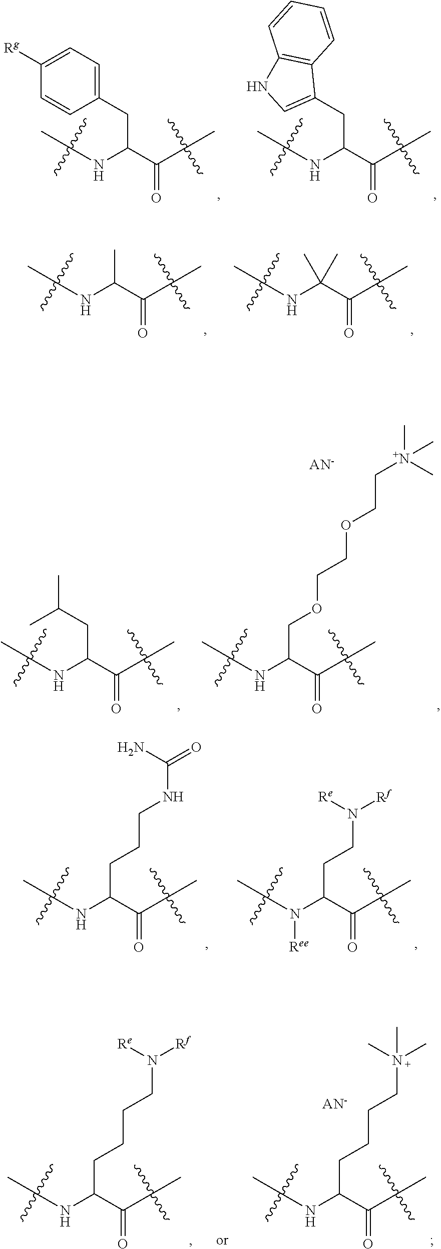

wherein:
   R^{e} is H, or —CH₃;
   R^{ee} is H;
   R^{f} is H, —CH₃, —C(CH₃)₃, —C(O)CH₃, —C(O)(CH₂CH₂O)₆CH₃, —(CH₂CH₂O)₆CH₃,

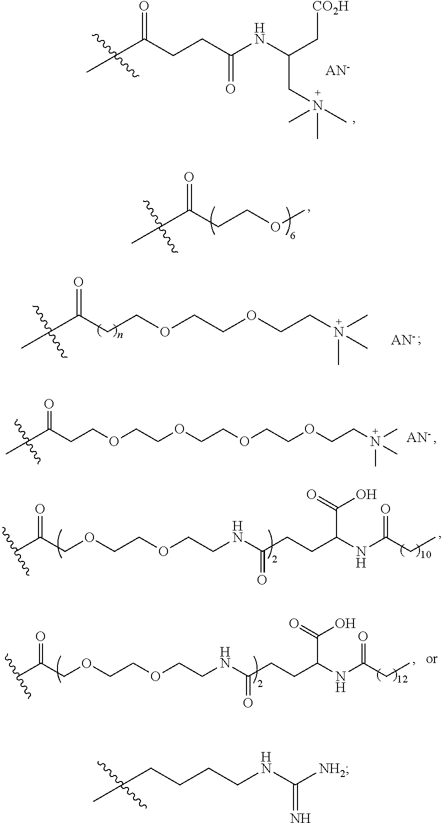

R^{g} is H, —OH, —C(O)NH₂, or —CH₂NHC(O)CH₃;
n is 0, or 1;
X12 is absent,

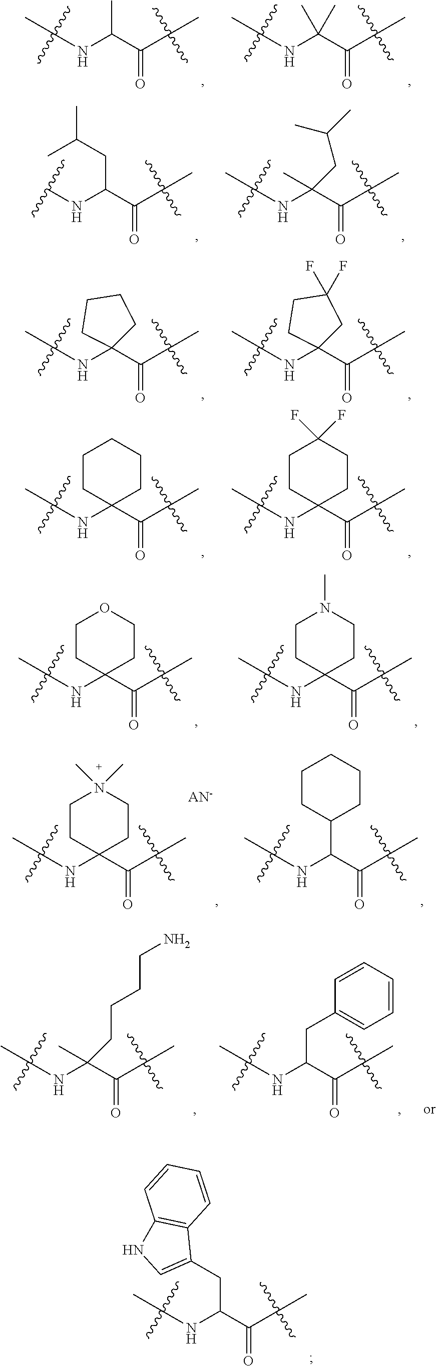

R⁶ is —CH₃, —CH₂CH₂C(O)R^{m}, —(CH₂)ₛNRⁿR^{o},

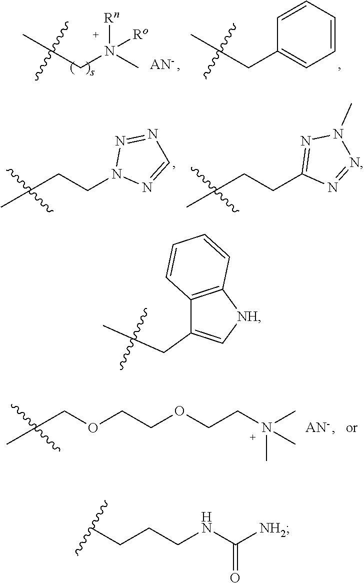

R⁷ is H, or —CH₃;
wherein:
   R^{m} is —OH, or

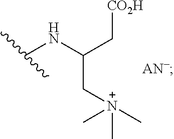 [SMILES: *NC(CC(=O)O)C[N+](C)(C)C]

Rⁿ is H, —CH₃, —CH₂CH(CH₃)₂, —C(O)CH₃, —C(O)(CH₂CH₂O)₆CH₃, —(CH₂CH₂O)₆CH₃, —C(O)(CH₂CH₂O)ₜCH₂CH₂R^{p},

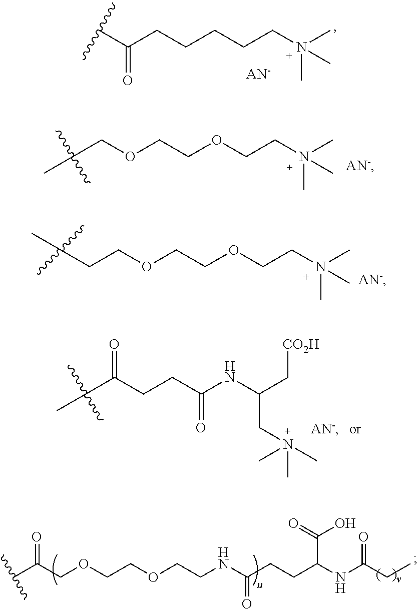

R^{o} is H, —CH₃, or —C(O)CH₃;
   wherein: PGP-723 C1
      R^{p} is NHC(O)CH₃, or

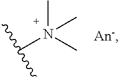

s is 1, 2, or 4;
t is 2, 6, 9, or 12;
u is 2 or 3;
v is 10 or 12;
R⁸ is —CH₂CH₂CH₂NHC(O)CH₃, —C(O)NH₂, or —C(O)NHCH₃;
X15 is absent,

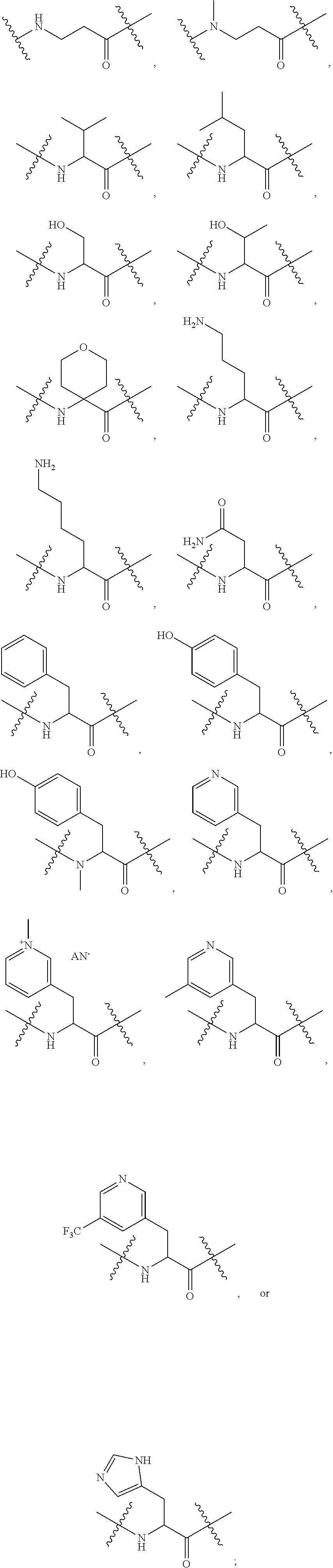

X16 is absent,

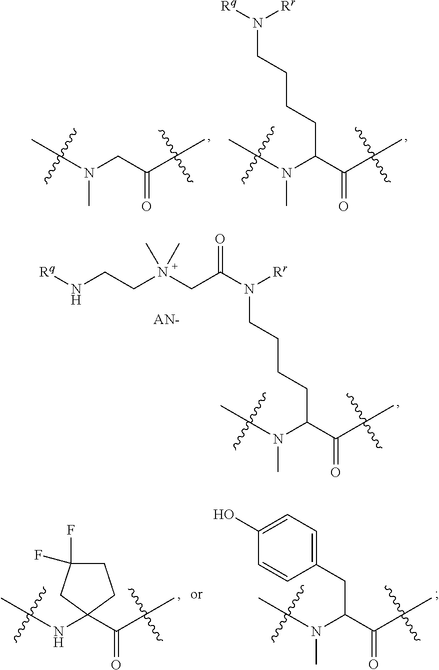

wherein:
   R^{q} is

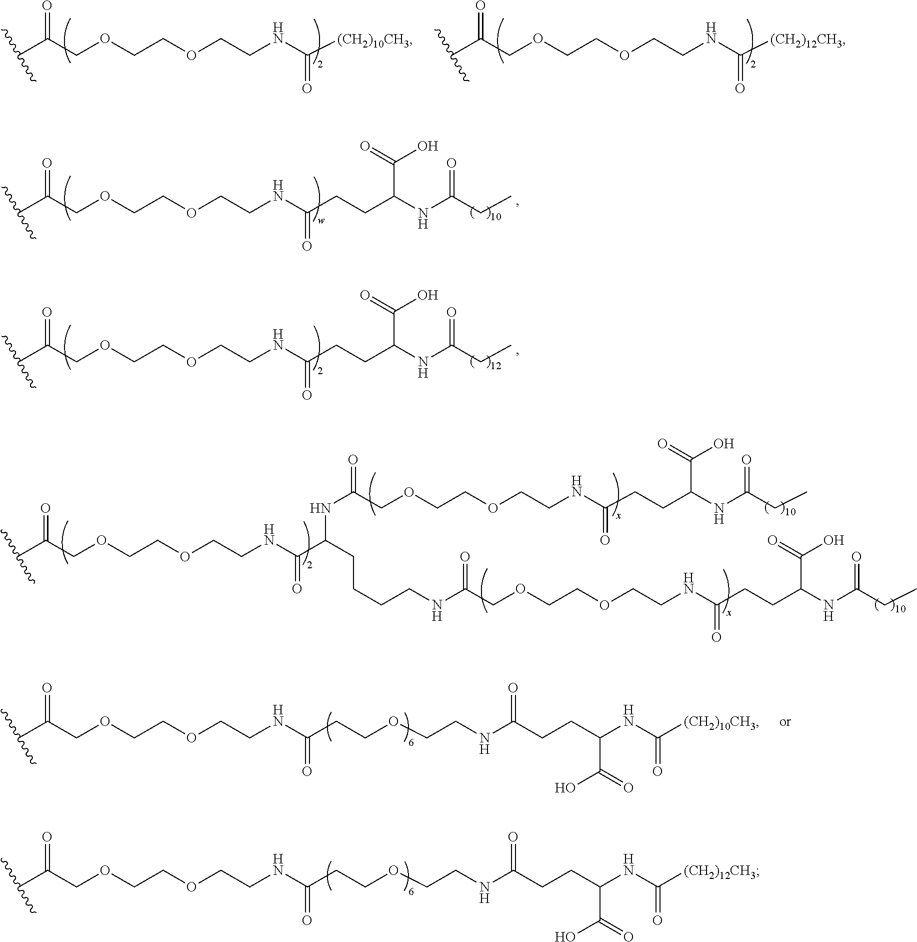

R^{r} is H;
w is 24, or 5;
x is 2 or 4; and
R⁹ is —NH₂, —NHCH₃, —N(CH₃)₂, —NH(CH₂CH₂O)₂CH₃, —N(CH₃)(CH₂CH₂O)₂CH₃, —NH(CH₂CH₂O)_{y}R^{s},

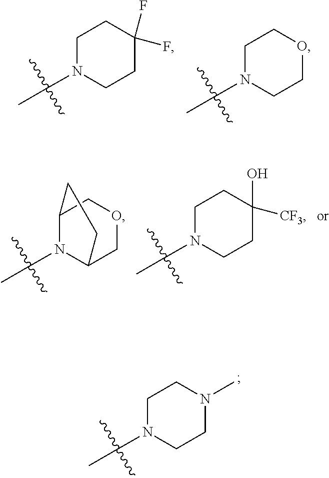

wherein:
   y is 2, 4, or 6;
   R^{s} is —CH₃, or

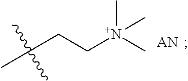 [SMILES: *CC[N+](C)(C)C]

wherein:
AN⁻as above defined for A, R, X3, X12, R1, R2, R5, R6, R8, R15, R16 and R^{a}, R^{b}, R^{d}, R^{f}, R^{h}, Rⁱ, R^{j}, R^{k}, R^{m}, Rⁿ, R^{o}, R^{s} is acetate, adipate, benzoate, benzenesulfonate, citrate, decanoate, chloride, lactate, maleate, methanesulfonate, oxalate, pivalate, propionate, succinate, sulfate, tartrate, or trifluoroacetate; or
a pharmaceutically acceptable salt thereof

37. In another aspect of the invention:

A compound of Formula (IX)

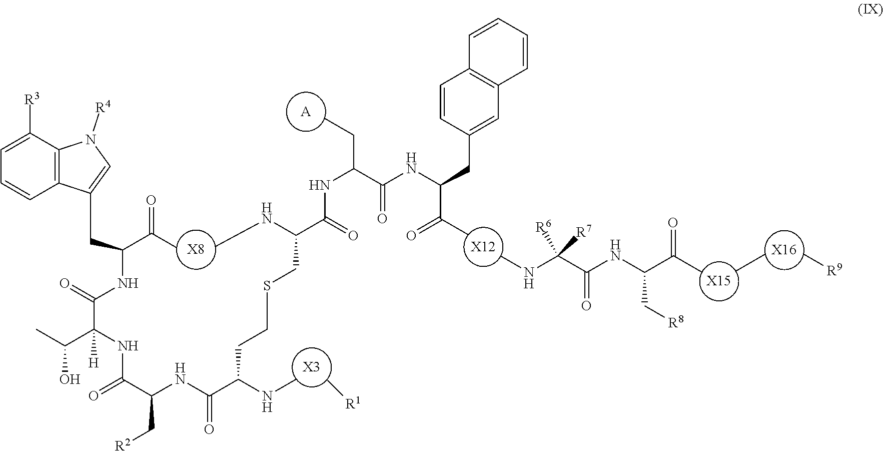

wherein:

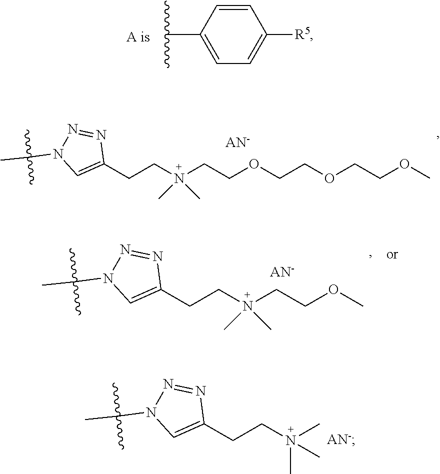

wherein:
   R⁵ is —OCHF₂, —O(CH₂)₉CO₂H,

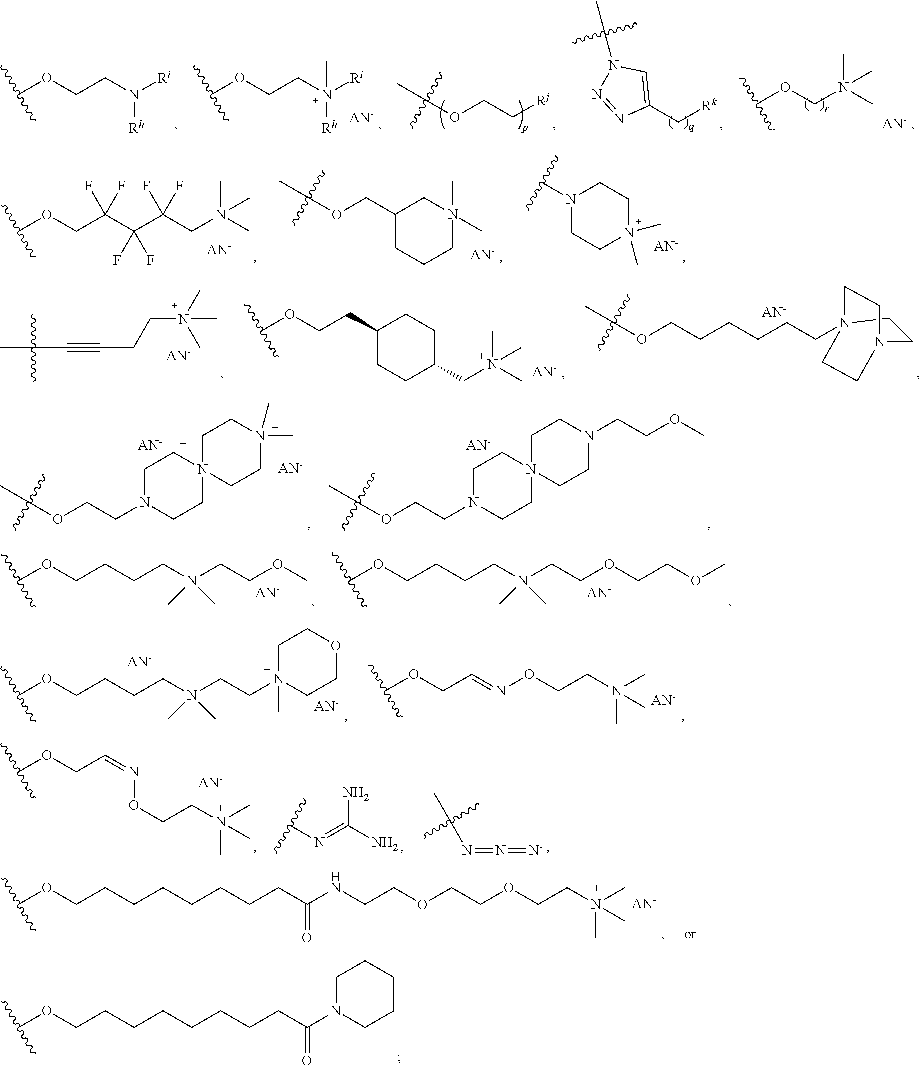

wherein:
   R^{h} is H, —CH₃, —C(O)CH₃, —C(NH)NH₂, —(CH₂)₃O(CH₂)₂OCH₃, —CH₂CH₂OCH₃, —(CH₂CH₂O)₃CH₃, —(CH₂CH₂O)₆CH₃,

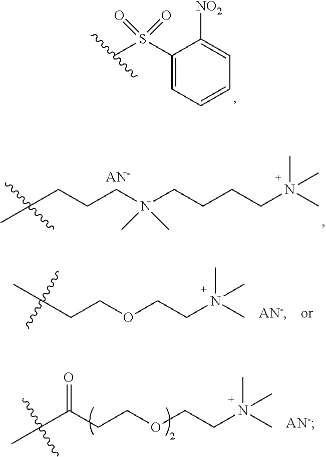

Rⁱ is H, —CH₃—(CH₂)₃O(CH₂)₂OCH₃,

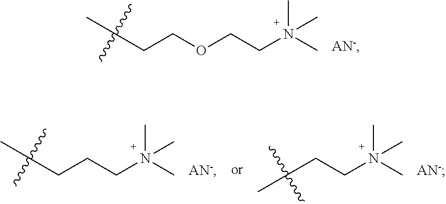

p is 1, 2 or 3;
R^{j} is

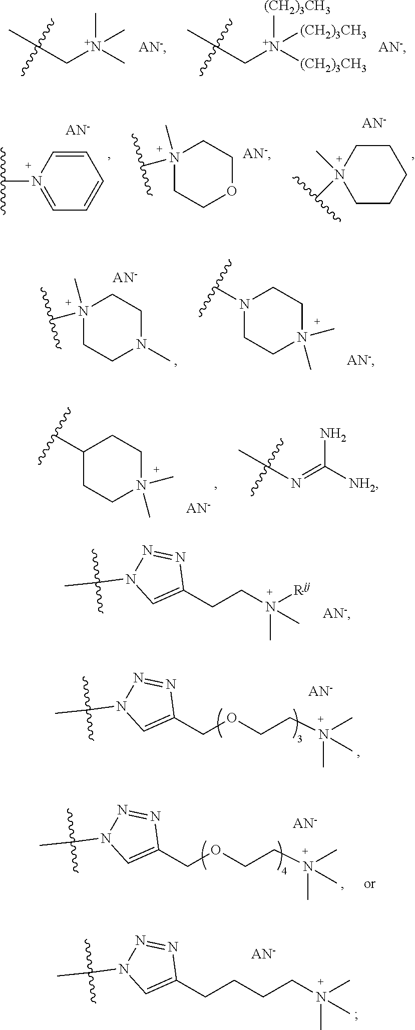

q is 1, 2, 3, 4, or 5;
R^{k} is —OCH₃,

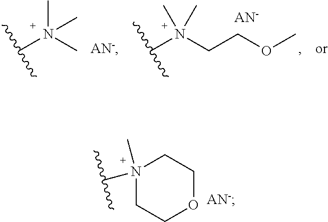

r is 3, 4, 5, 6, or 8;
wherein:
   R^{jj} is H, —(CH₂)₇CH₃, —(CH₂)₁₅CH₃, —(CH₂)₂OCH₃, or —(CH₂CH₂O)₃CH₃;
R¹ is —C(O)C(1-2)alkyl, —C(O)CF₃, —COCH₂CF₃, —CO(CH₂)₅N(CH₃)₃⁺AN⁻, —CH₂CH₂NHC(O)CH₂O(CH₂CH₂O)₂CH₃, —CH₂CH₂NHC(O)(CH₂CH₂O)₆CH₃,

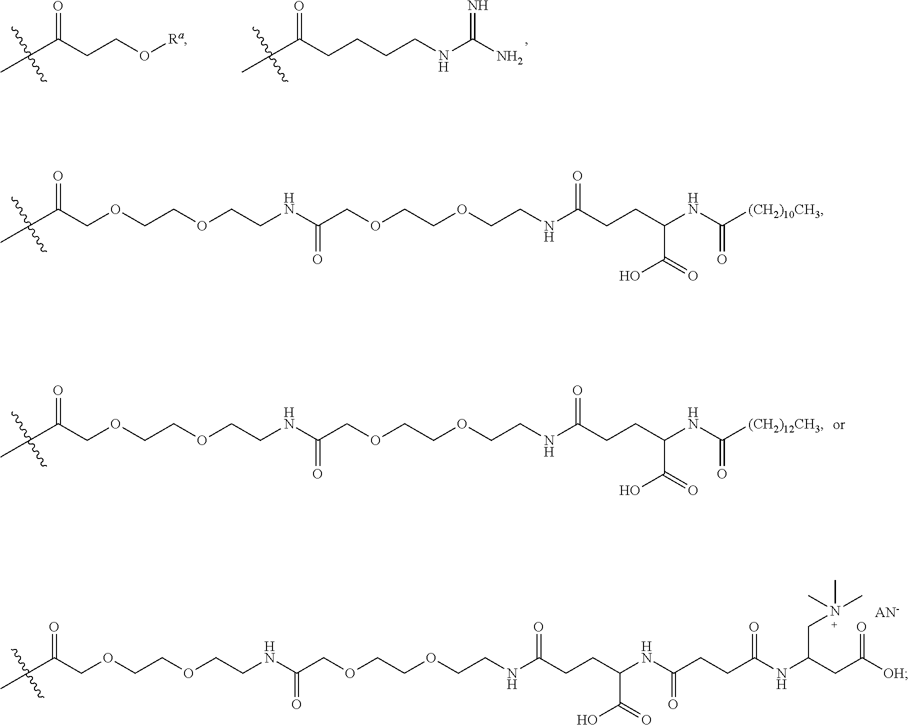

wherein:
   R^{a} is |,

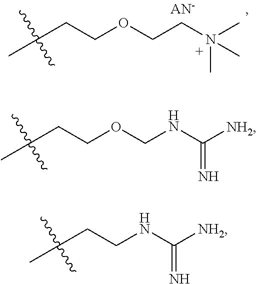

CH₂CH₂OCH₂CH₂N(CH₃)₂,
—(CH₂CH₂O)₃CH₂CH₂N(CH₃)₂, —(CH₂CH₂O)₃CH₂CH₂N(CH₃)₃ ⁺AN⁻, —(CH₂CH₂O)₅CH₂CH₂NC(O)CH₃, —(CH₂CH₂O)₈CH₂CH₂NC(O)CH₃, or —(CH₂CH₂O)₁₁CH₂CH₂NC(O)CH₃;
X3 is a direct bond, (D) arginine, (D) lysine substituted with R^{b},

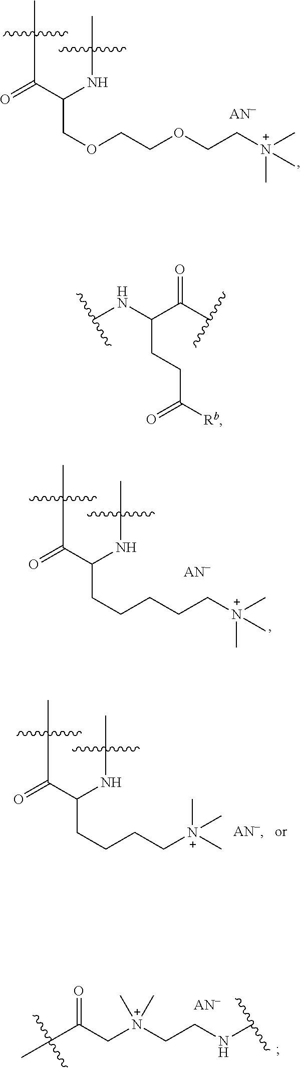

wherein:
   R^{b} is

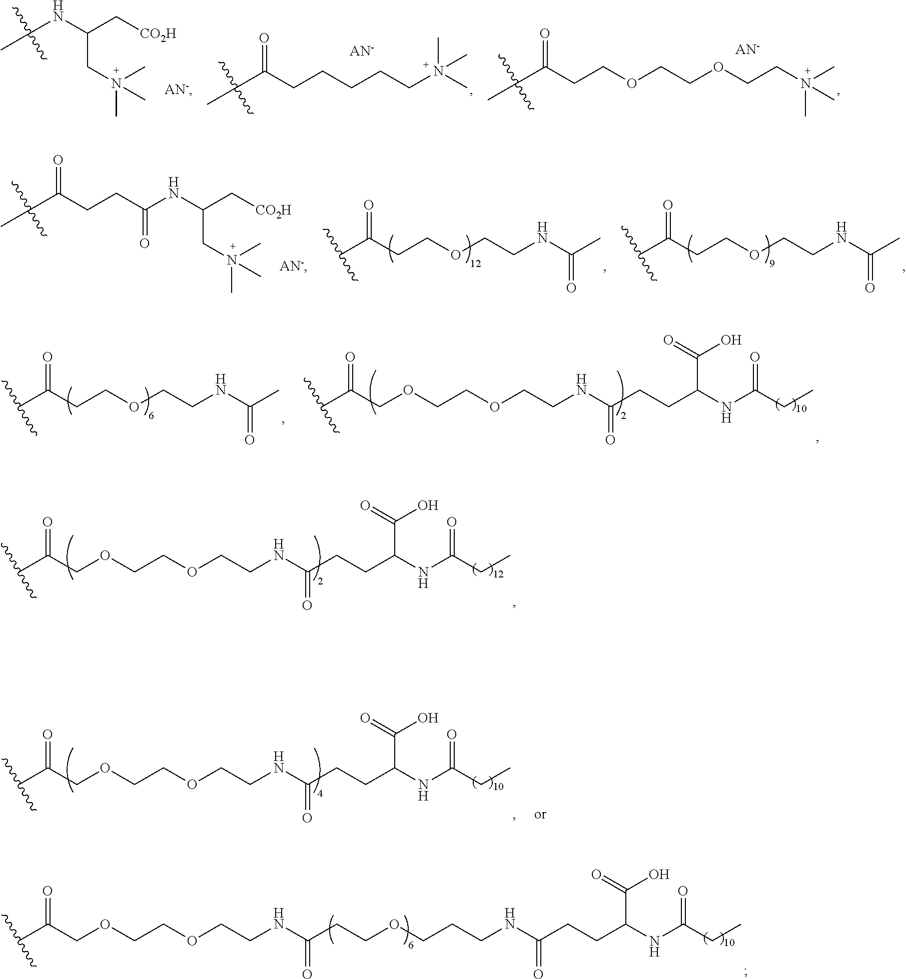

R² is H, —CH(CH₃)₂,

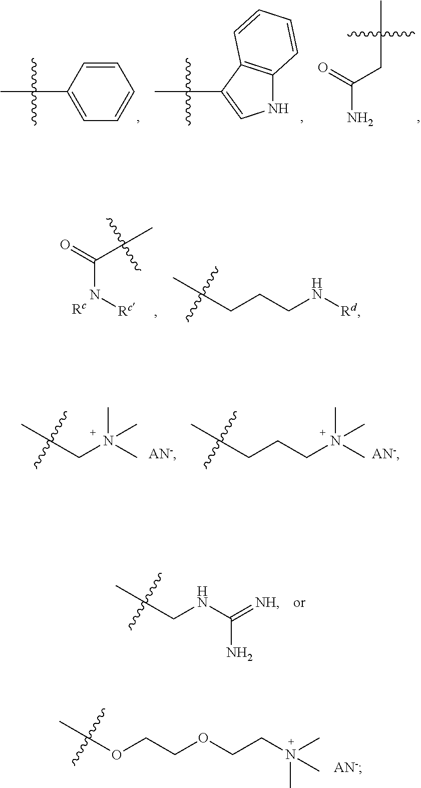

wherein:
   R^{c} is H, or —CH₃;
   R^{c}′ is H, or —CH₃;
   R^{d} is

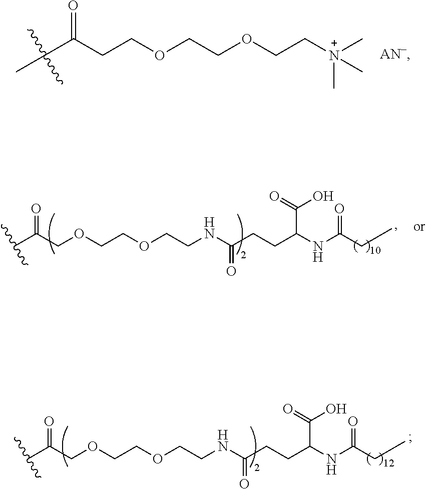

R³ is H, —CH₃, —CF₃, or

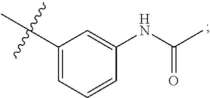 [SMILES: *c1cccc(NC(C)=O)c1]

R⁴ is H, or —CH₃;

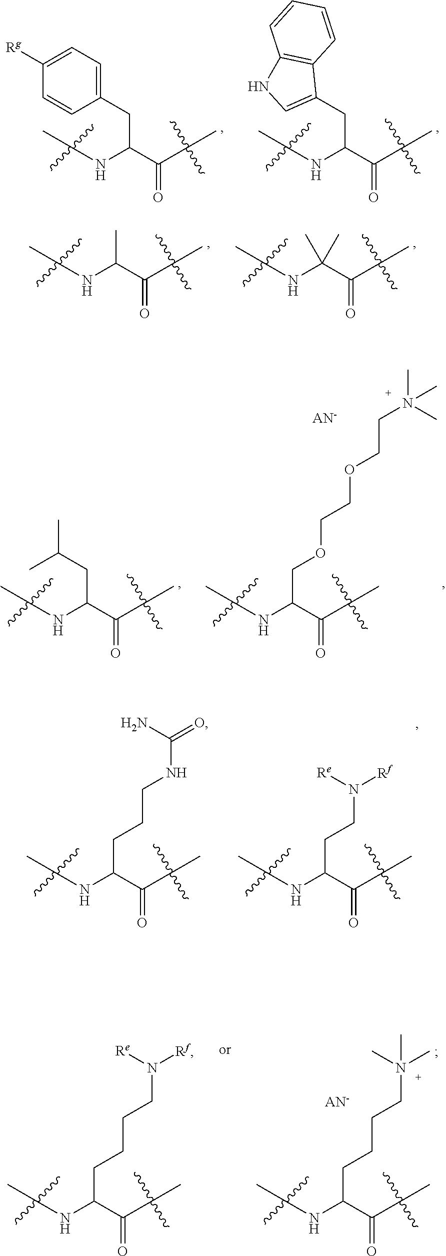

wherein:
   R^{e} is H, or —CH₃;
   R^{ee} is H;
   R^{f} is H, —CH₃, —C(CH₃)₃, —C(O)CH₃, —C(O)(CH₂CH₂O)₆CH₃, —(CH₂CH₂O)₆CH₃,

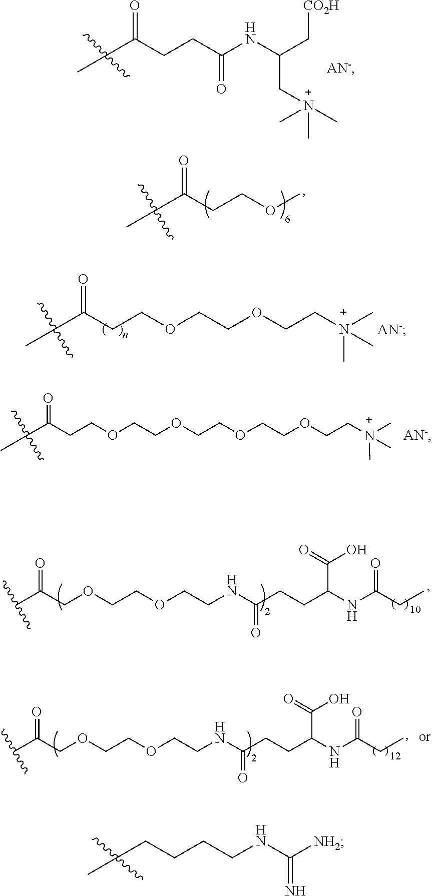

R^{g} is H, —OH, —C(O)NH₂, or —CH₂NHC(O)CH₃;
n is 0, or 1;
X12 is absent,

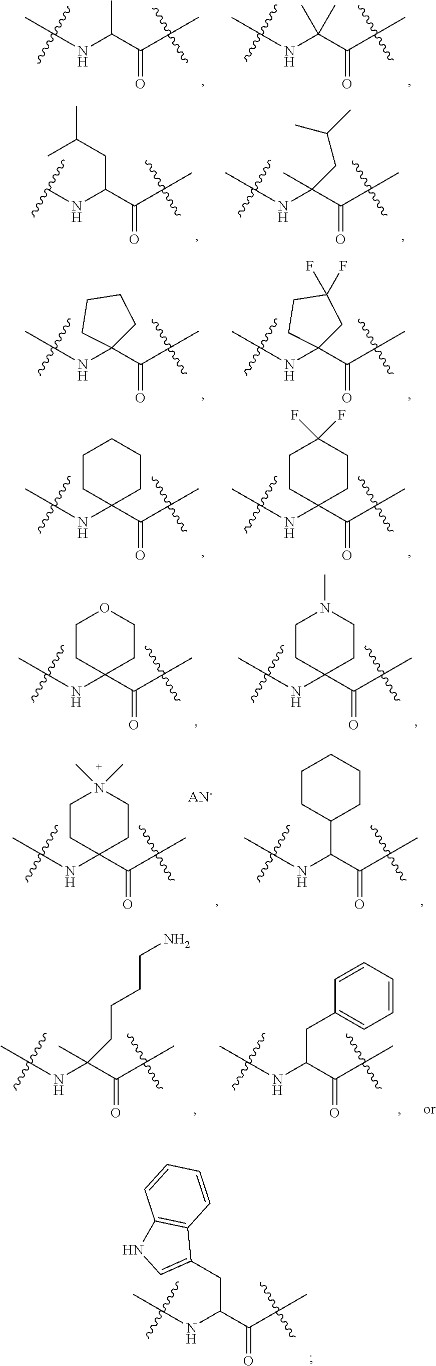

R⁶ is —CH₃, —CH₂CH₂C(O)R^{m}, —(CH₂)ₛNRⁿR^{o},

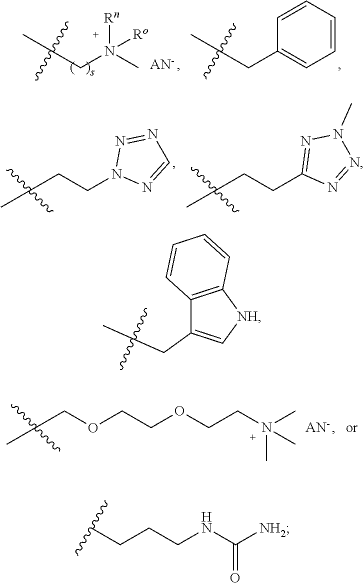

R⁷ is H, or —CH₃;
wherein:
   R^{m} is —OH, or

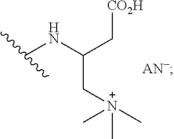 [SMILES: *NC(CC(=O)O)C[N+](C)(C)C]

Rⁿ is H, —CH₃, —CH₂CH(CH₃)₂, —C(O)CH₃, —C(O)(CH₂CH₂O)₆CH₃, —(CH₂CH₂O)₆CH₃, —C(O)(CH₂CH₂O)ₜCH₂CH₂R^{p},

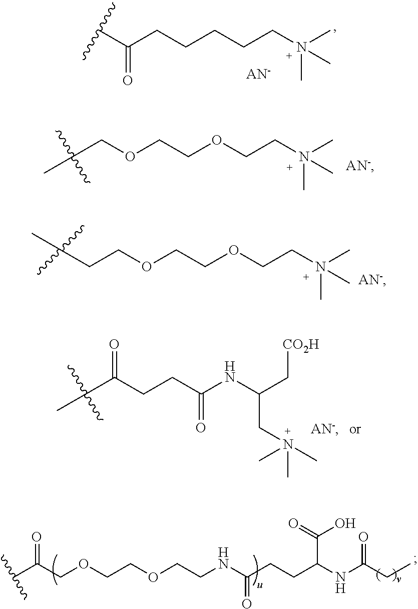

R^{o} is H, —CH₃, or _C(O)CH₃;
wherein:
   R^{p} is NHC(O)CH₃, or

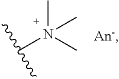

s is 1,2, or 4;
t is 2, 6, 9, or 12;
u is 2 or 3;
v is 10 or 12;
R⁸ is —CH₂CH₂CH₂NHC(O)CH₃, —C(O)NH₂, or —C(O)NHCH₃;
X15 is absent,

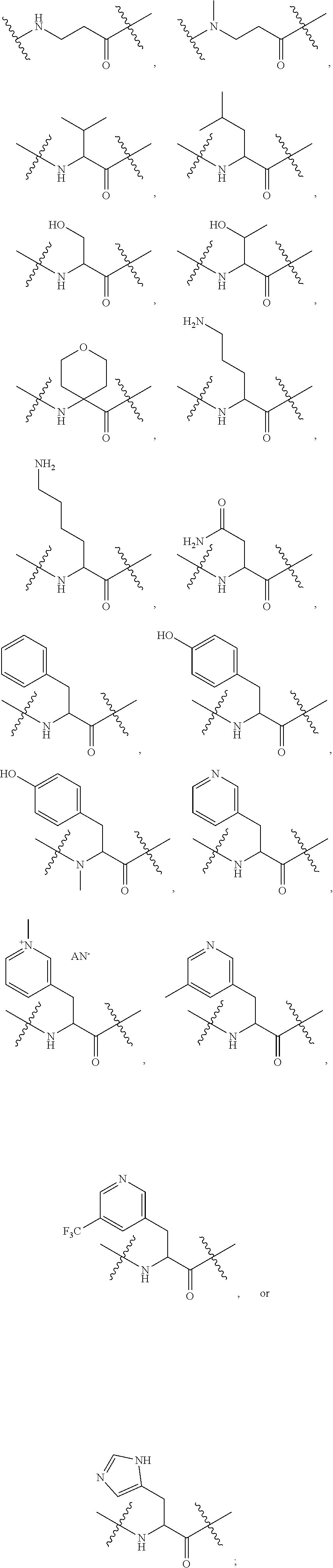

X16 is absent,

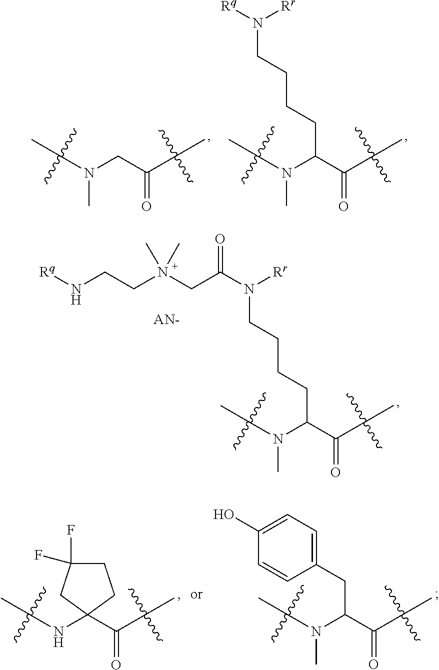

wherein:
   R^{q} is

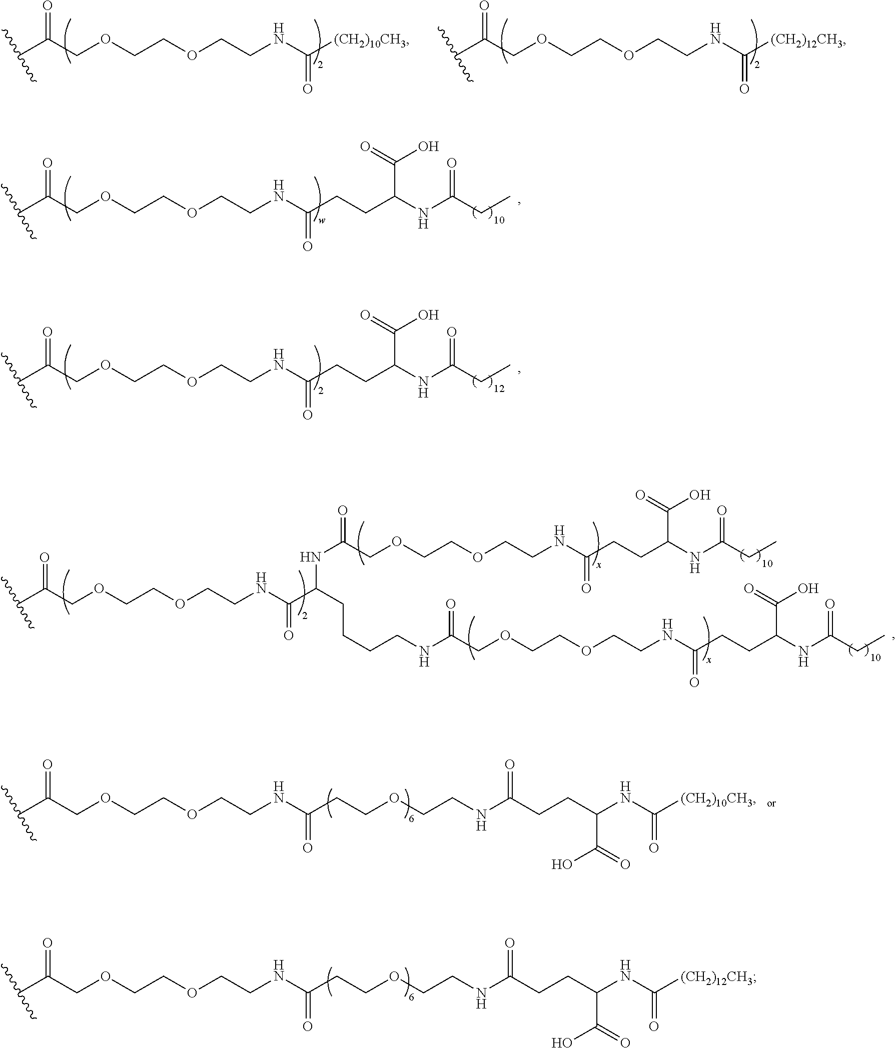

R^{r} is H;
w is 24, or 5;
x is 2 or 4; and
R⁹ is —NH₂, —NHCH₃, —N(CH₃)₂, —NH(CH₂CH₂O)₂CH₃, —N(CH₃)(CH₂CH₂O)₂CH₃, —NH(CH₂CH₂O)_{y}R^{s},

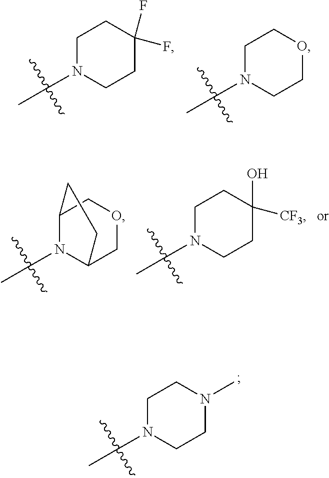

wherein:
   y is 2, 4, or 6;
   R^{s} is —CH₃, or

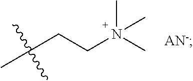

wherein:
AN⁻as above defined for A, R, X3, X12, R1, R2, R5, R6, R8, R15, R16 and R^{a}, R^{b}, R^{d}, R^{f}, R^{h}, Rⁱ, R^{j}, R^{k}, R^{m}, Rⁿ, R^{o}, R^{s} is acetate, adipate, benzoate, benzenesulfonate, citrate, decanoate, chloride, lactate, maleate, methanesulfonate, oxalate, pivalate, propionate, succinate, sulfate, tartrate, or trifluoroacetate; or
a pharmaceutically acceptable salt thereof.

In another aspect of the invention

38. A compound of Formula (X)

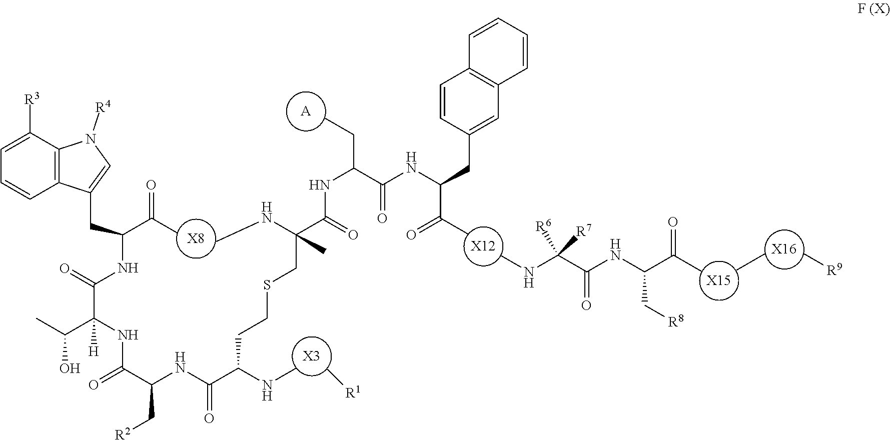

wherein:
A is

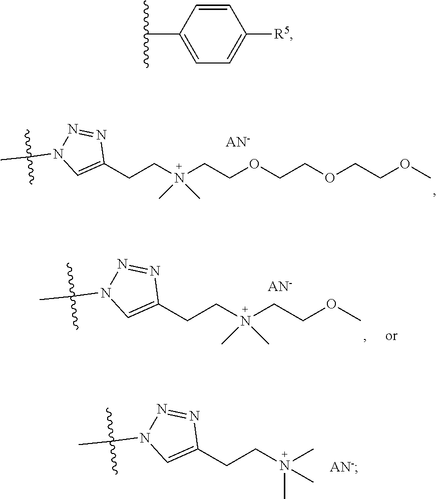

wherein
   R⁵ is —OCHF₂, —O(CH₂)₉CO₂H,

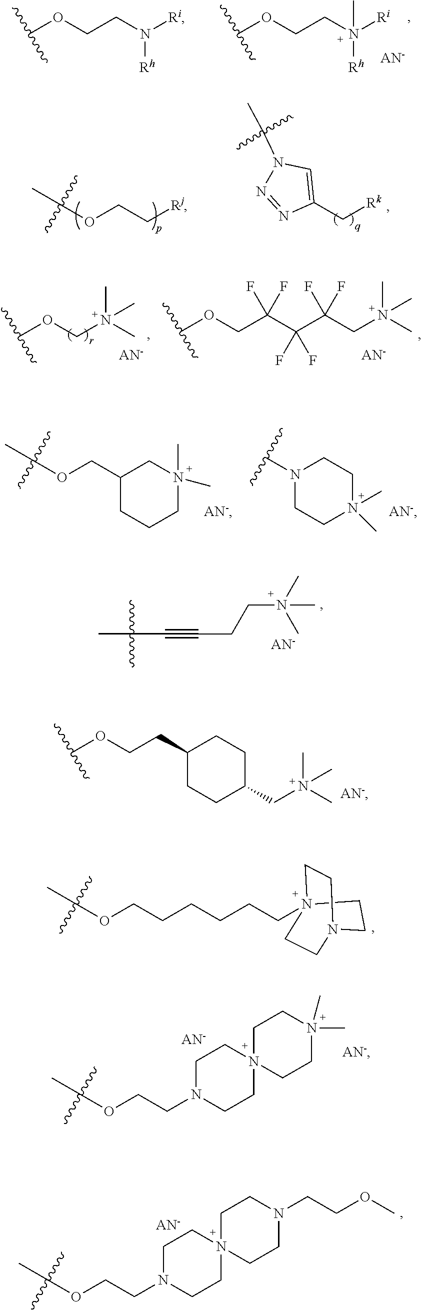

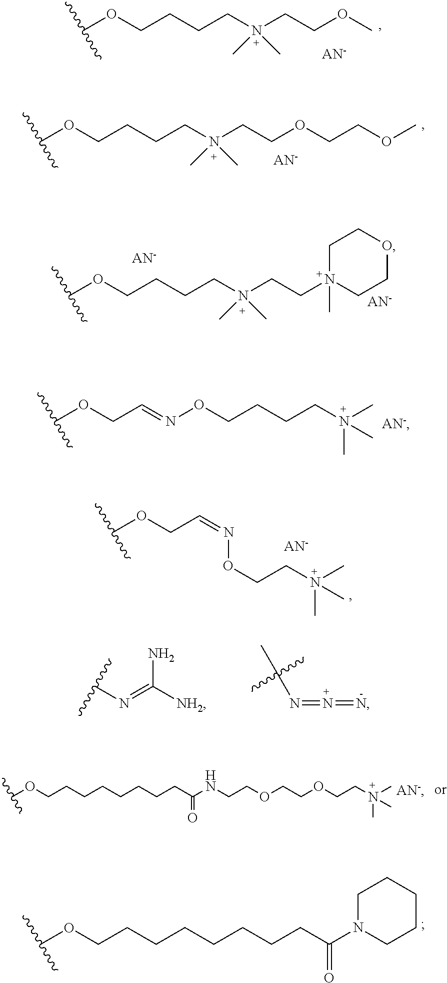

wherein:
   R^{h} is H, —CH₃, —C(O)CH₃, —C(NH)NH₂, —(CH₂)₃O(CH₂)₂OCH₃, —CH₂CH₂OCH₃, —(CH₂CH₂O)₃CH₃, —(CH₂CH₂O)₆CH₃,

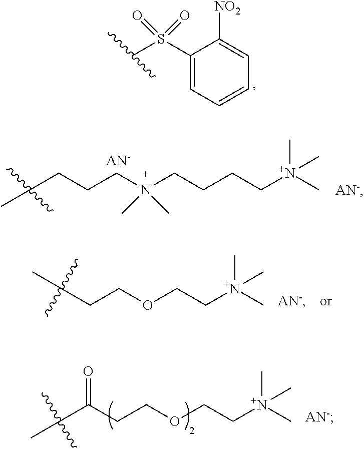

Rⁱ is H, —CH₃, —(CH₂)₃O(CH₂)₂OCH₃,

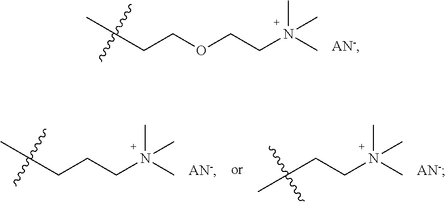

p is 1, 2 or 3;
R^{j} is

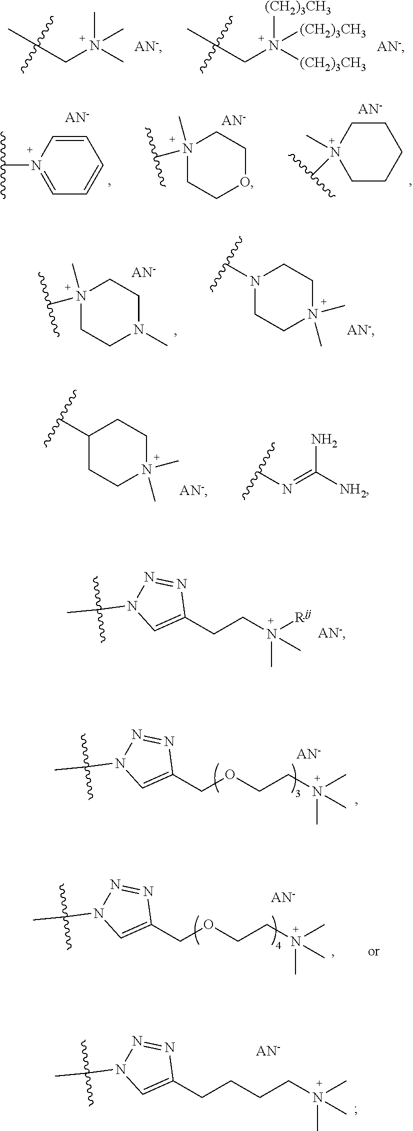

q is 1, 2, 3, 4, or 5;
R^{k} is —OCH₃,

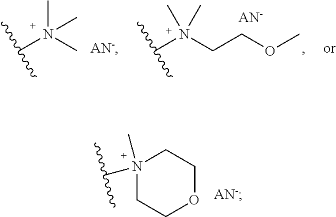

r is 3, 4, 5, 6, or 8;
wherein:
   R^{jj} is H, —(CH₂)₇CH₃, —(CH₂)₁₅CH₃, —(CH₂)₂OCH₃, or —(CH₂CH₂O)₃CH₃;
R¹ is —C(O)C(1-2)alkyl, —C(O)CF₃, —COCH₂CF₃, —CO(CH₂)₅N(CH₃)₃⁺AN⁻, —CH₂CH₂NHC(O)CH₂O(CH₂CH₂O)₂CH₃, —CH₂CH₂NHC(O)(CH₂CH₂O)₆CH₃,

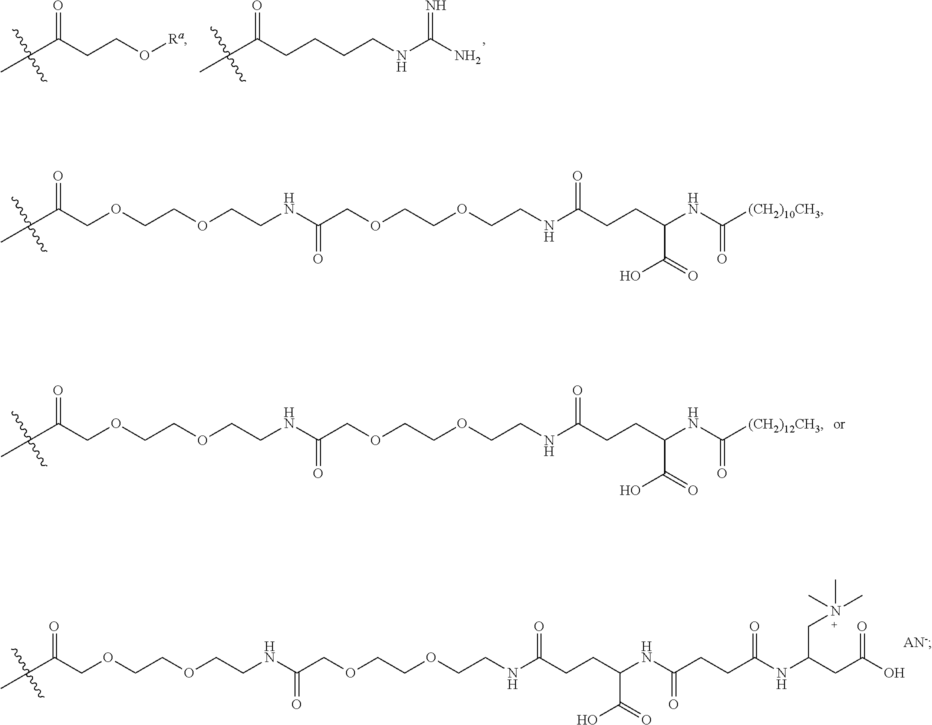

wherein:
   R^{a} is

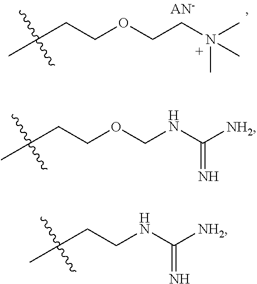

CH₂CH₂OCH₂CH₂N(CH₃)₂,
—(CH₂CH₂O)₃CH₂CH₂N(CH₃)₂, —(CH₂CH₂O)₃CH₂CH₂N(CH₃)₃ ⁺AN⁻, —(CH₂CH₂O)₅CH₂CH₂NC(O)CH₃, —(CH₂CH₂O)₈CH₂CH₂NC(O)CH₃, or —(CH₂CH₂O)₁₁CH₂CH₂NC(O)CH₃;
X3 is a direct bond, (D) arginine, (D) lysine substituted with R^{b},

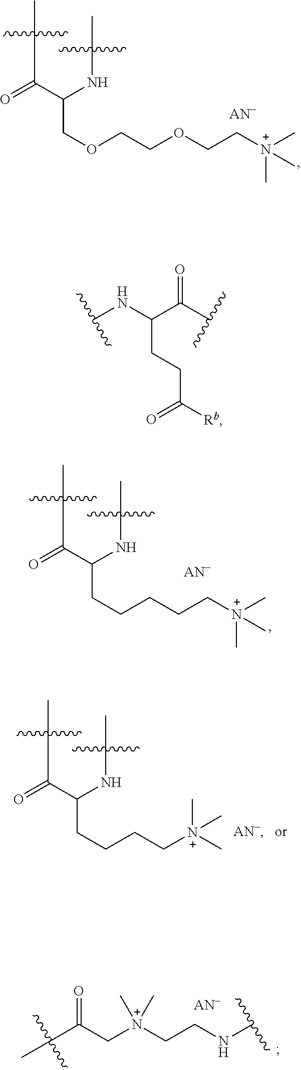

wherein:
   R^{b} is

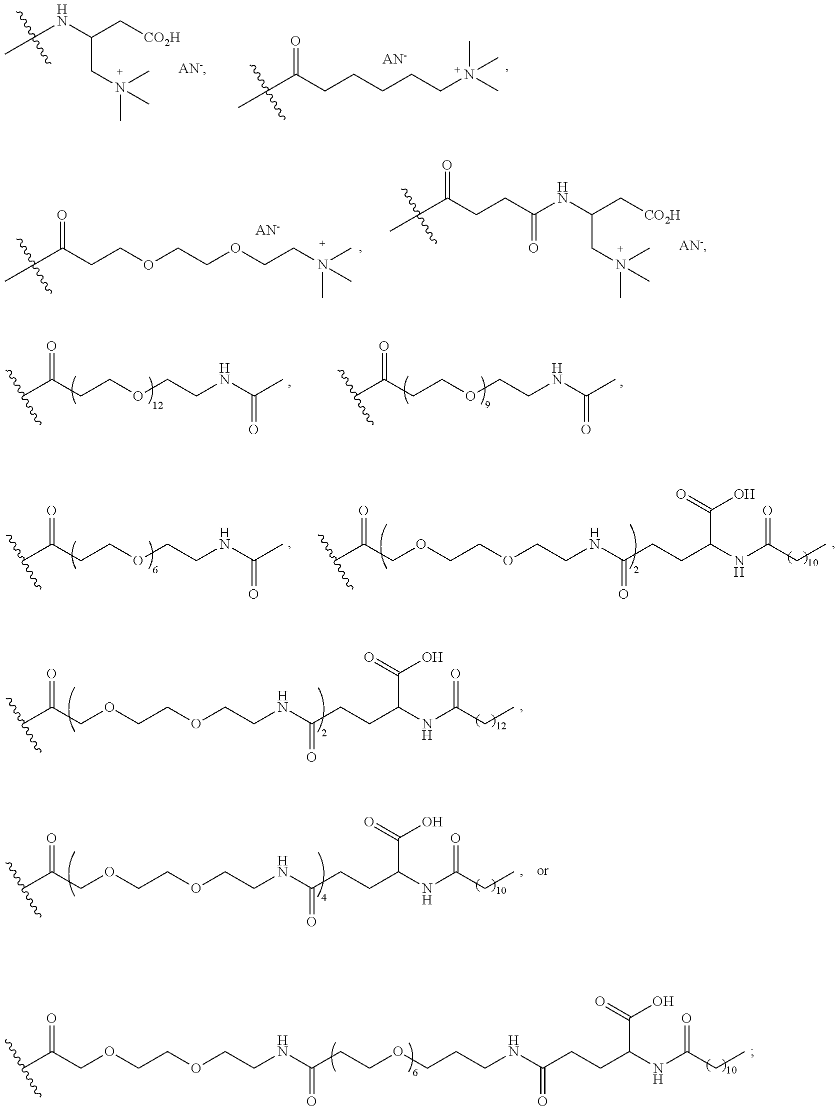

R² is H, —CH(CH₃)₂,

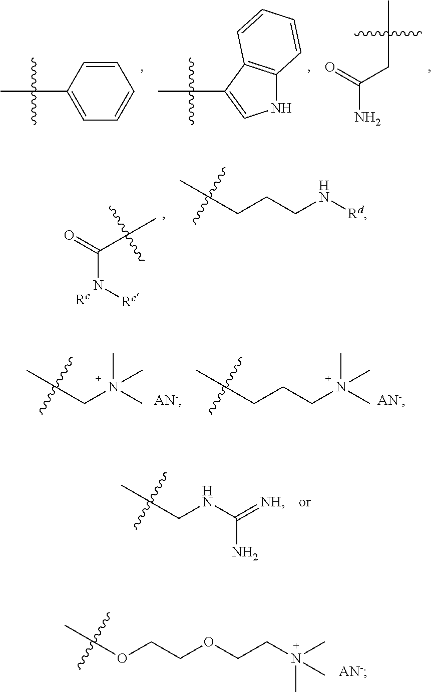

wherein:
   R^{c} is H, or —CH₃;
   R^{c}′ is H, or —CH₃;
   R^{d} is

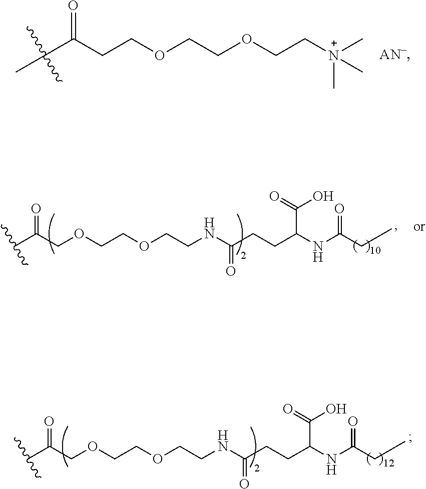

R³ is H, —CH₃,

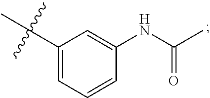 [SMILES: *c1cccc(NC(C)=O)c1]

R⁴ is H, or —CH₃;
X8 is

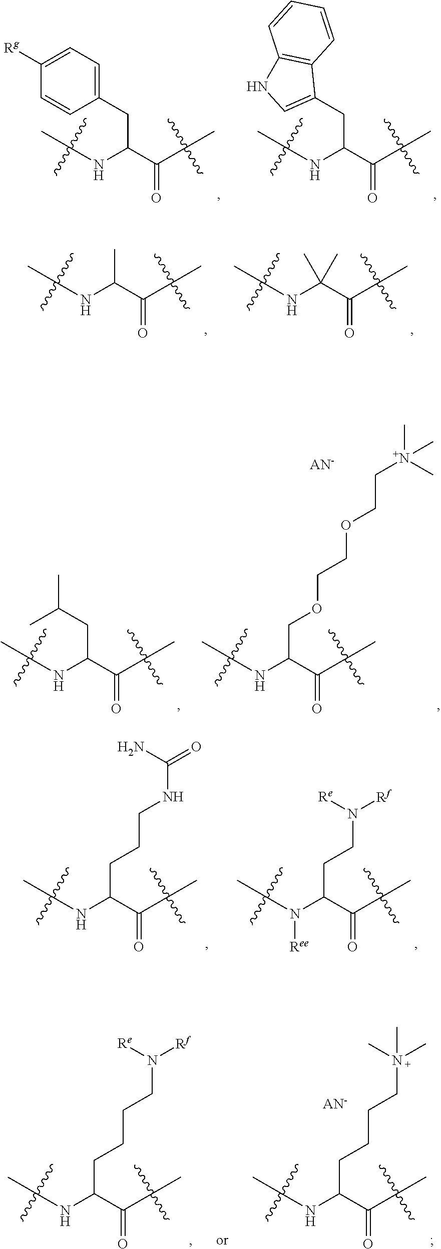

wherein:
   R^{e} is H, or —CH₃;
   R^{ee} is H;
   R^{f} is H, —CH₃, —C(CH₃)₃, —C(O)CH₃, —C(O)(CH₂CH₂O)₆CH₃, —(CH₂CH₂O)₆CH₃,

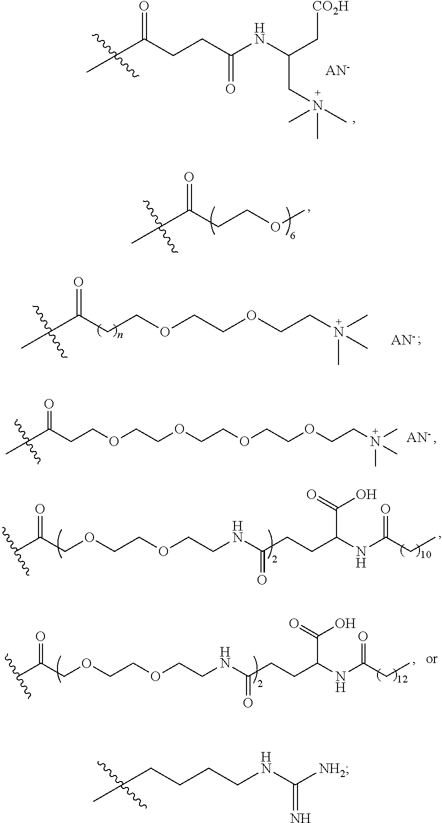

R^{g} is H, —OH, —C(O)NH₂, or —CH₂NHC(O)CH₃;
n is 0, or 1;
X12 is absent,

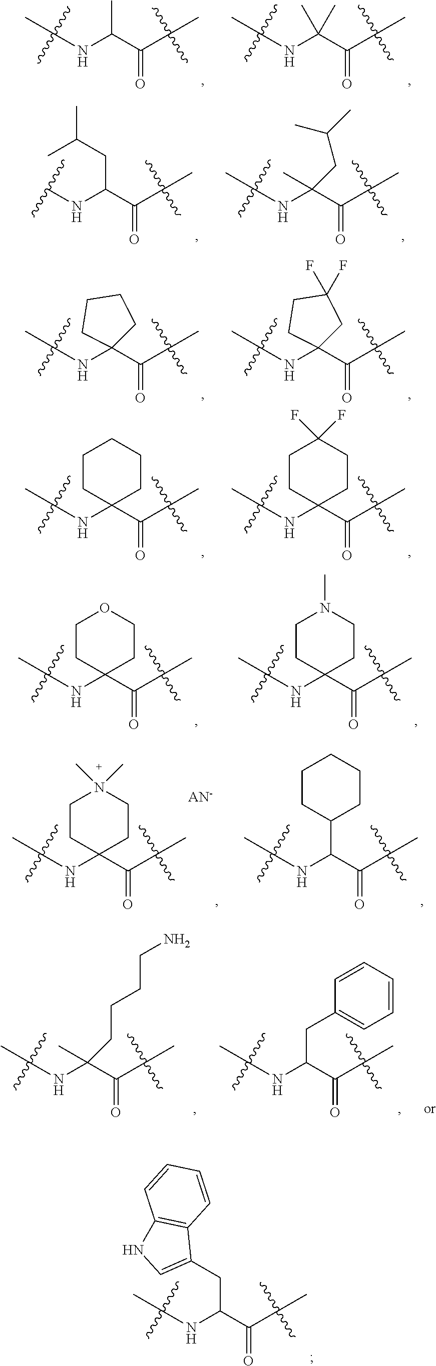

R⁶ is —CH₃—CH₂CH₂C(O)R^{m}, —(CH₂)ₛNⁿR^{o},

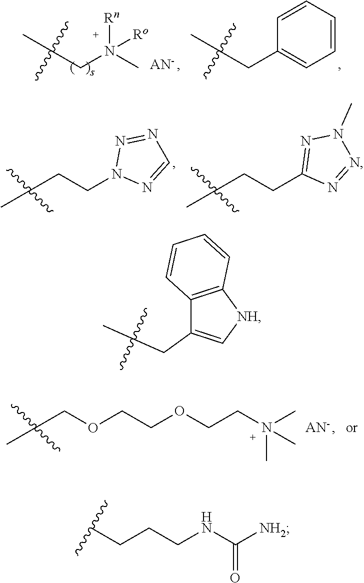

R⁷ is H, or —CH₃;
wherein:
   R^{m} is —OH, or

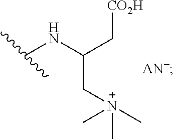 [SMILES: *NC(CC(=O)O)C[N+](C)(C)C]

Rⁿ is H, —CH₃, —CH₂CH(CH₃)₂, —C(O)CH₃, —C(O)(CH₂CH₂O)₆CH₃, —(CH₂CH₂O)₆CH₃, —C(O)(CH₂CH₂O)ₜCH₂CH₂R^{p},

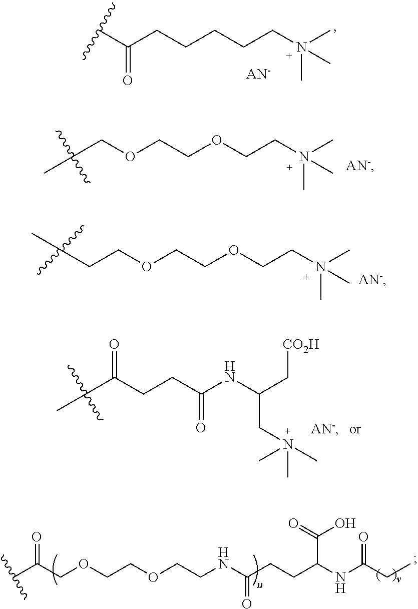

R^{o} is H, —CH₃, or —C(O)CH₃;
wherein:
   R^{p} is NHC(O)CH₃, or

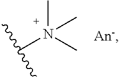

s is 1, 2, or 4;
t is 2, 6, 9, or 12;
u is 2 or 3;
v is 10 or 12;
R⁸ is —CH₂CH₂CH₂NHC(O)CH₃, —C(O)NH₂, or —C(O)NHCH₃;
X15 is absent,

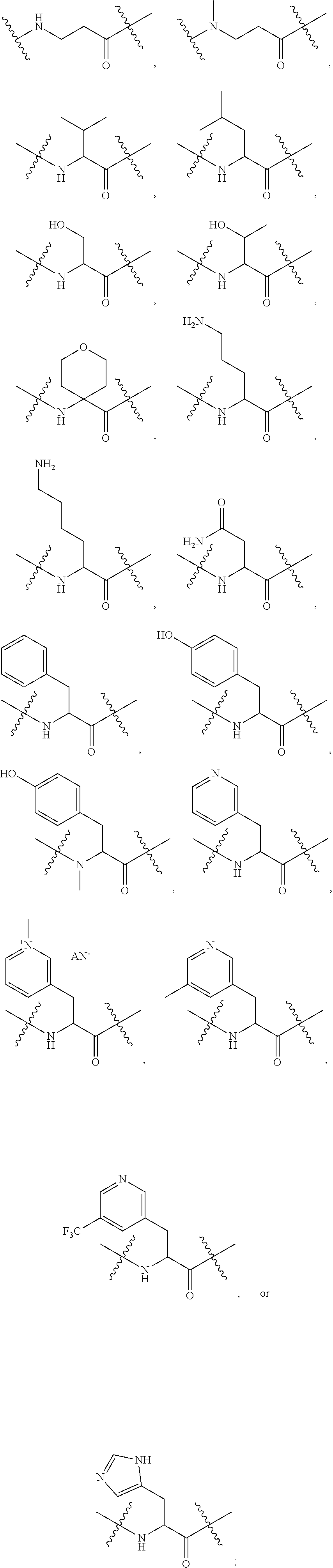

X16 is absent,

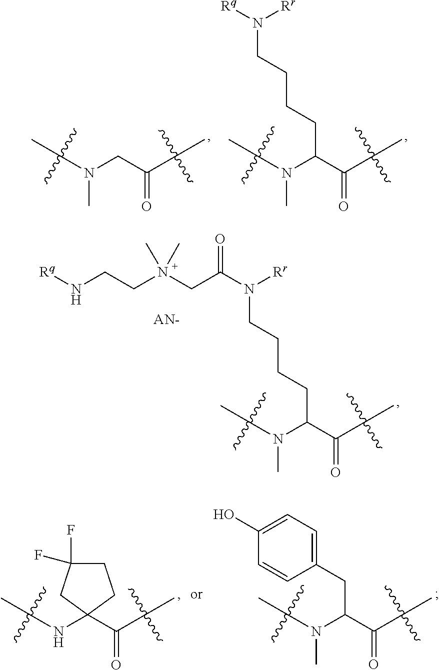

wherein
   R^{q} is

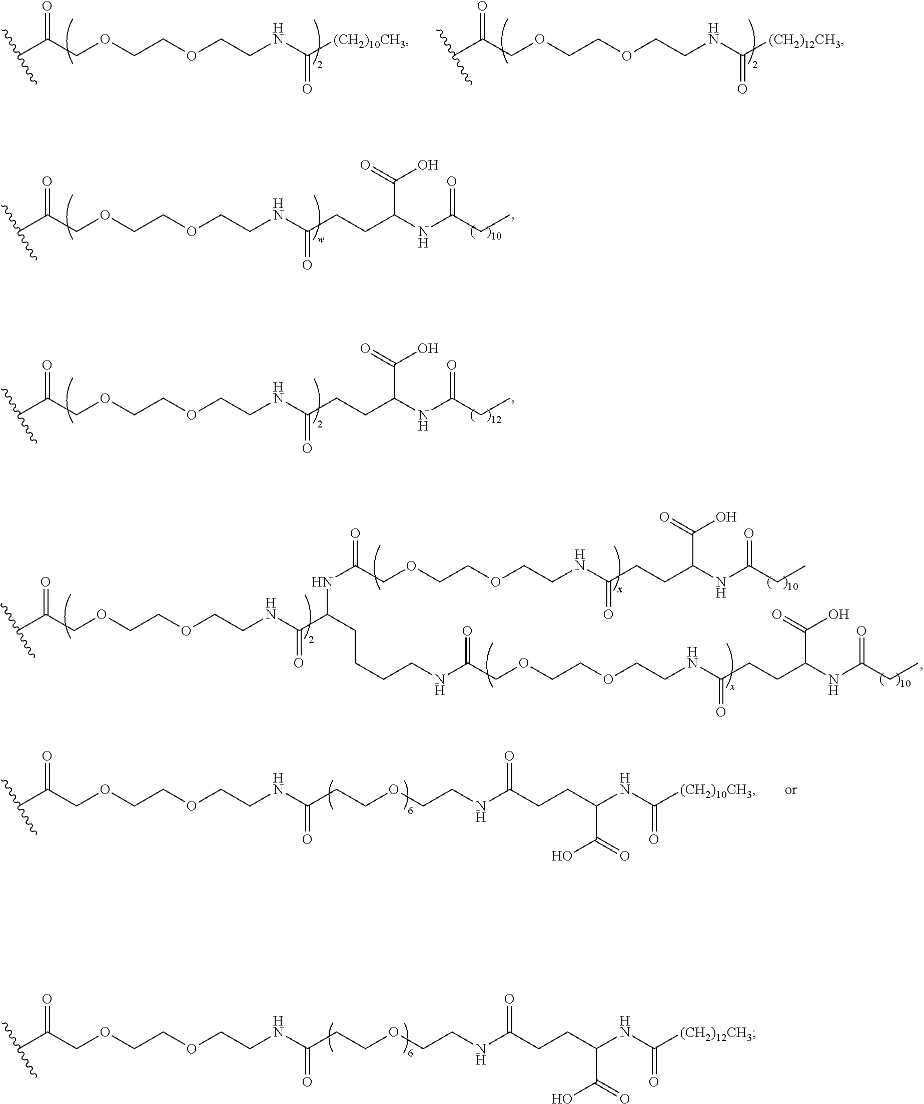

R^{r} is H;
w is 2 4, or 5;
x is 2 or 4; and
R⁹ is —NH₂, —NHCH₃, —N(CH₃)₂, —NH(CH₂CH₂O)₂CH₃, —N(CH₃)(CH₂CH₂O)₂CH₃, —NH(CH₂CH₂O)_{y}R^{s},

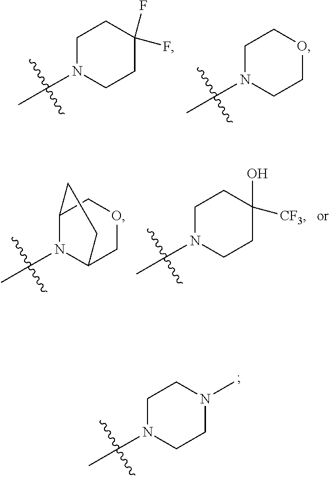

wherein:
   y is 2, 4, or 6;
   R^{s} is —CH₃, or

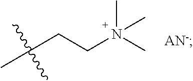

wherein:
AN⁻as above defined for A, R, X3, X12, R1, R2, R5, R6, R8, R15, R16 and R^{a}, R^{b}, R^{d}, R^{f}, R^{h}, Rⁱ, R^{j}, R^{k}, R^{m}, Rⁿ, R^{o}, R^{s} is acetate, adipate, benzoate, benzenesulfonate, citrate, decanoate, chloride, lactate, maleate, methanesulfonate, oxalate, pivalate, propionate, succinate, sulfate, tartrate, or trifluoroacetate; or
a pharmaceutically acceptable salt thereof.

In another aspect of the invention:

39. The compound of aspect 14, wherein X15 is 3-pyridyl alanine; or
a pharmaceutically acceptable salt thereof.

### Enumerated Clauses

The present disclosure further includes the following enumerated clauses.

### Clause 1

1. A compound of Formula (I):

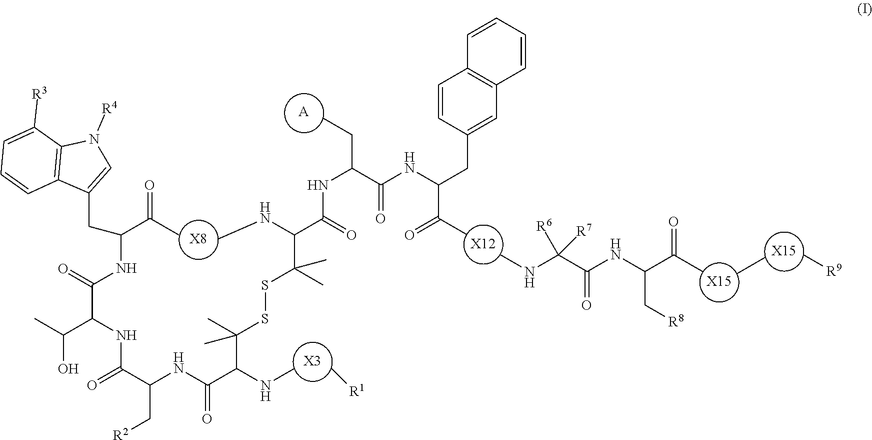

wherein:
A is

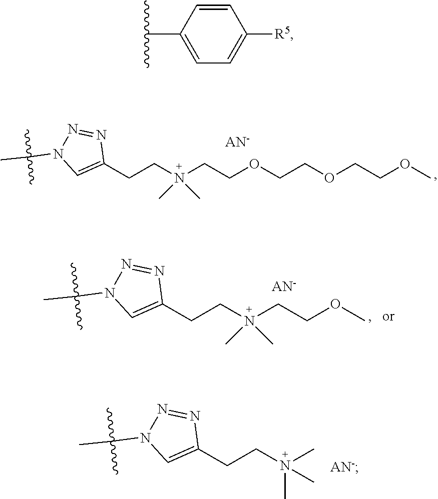

wherein:
   R^{s} is —OCHF₂, —O(CH₂)₉CO₂H,

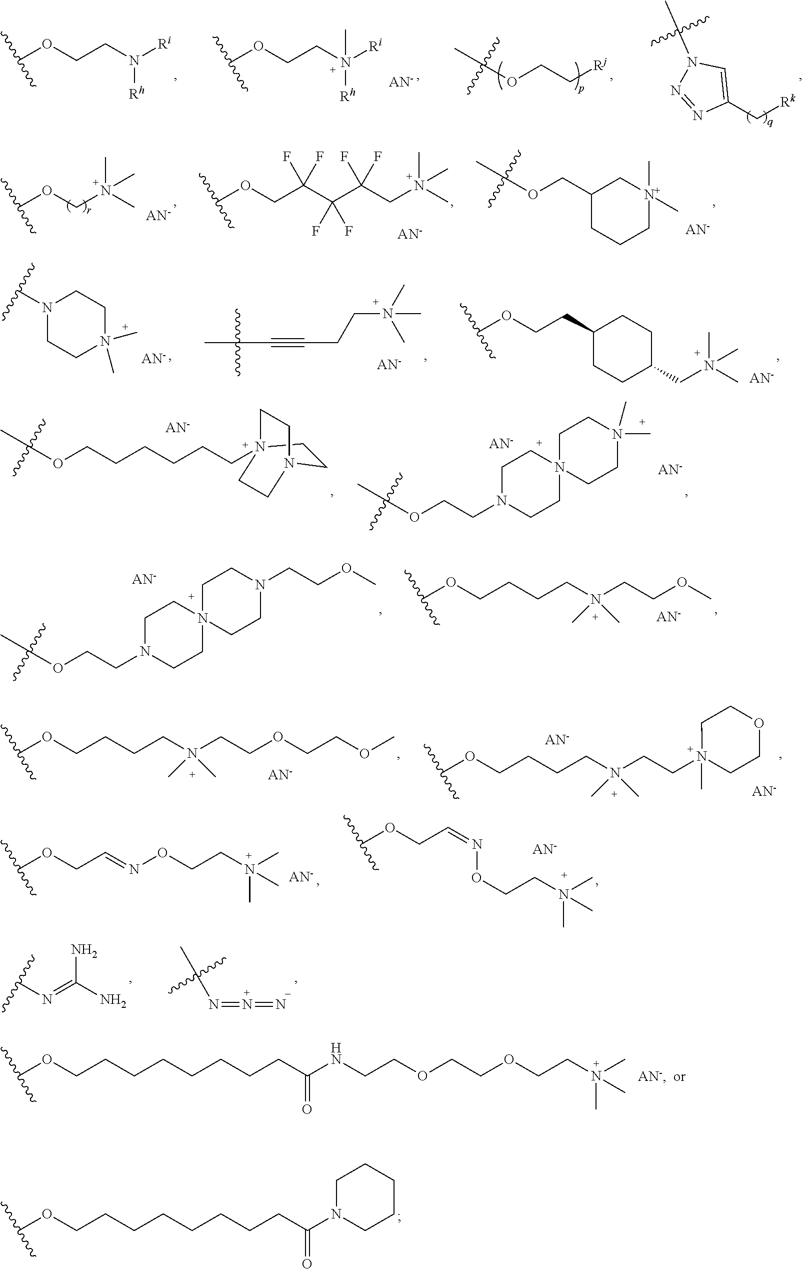

wherein:
   R^{h} is H, —CH₃, —C(O)CH₃, —C(NH)NH₂, —(CH₂)₃O(CH₂)₂OCH₃, —CH₂CH₂OCH₃, —(CH₂CH₂O)₃CH₃, —(CH₂CH₂O)₆CH₃,

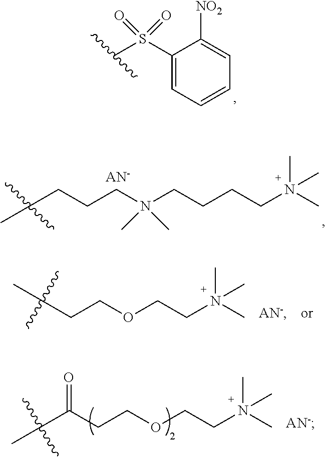

Rⁱ is H, —CH₃, —(CH₂)₃O(CH₂)₂OCH₃,

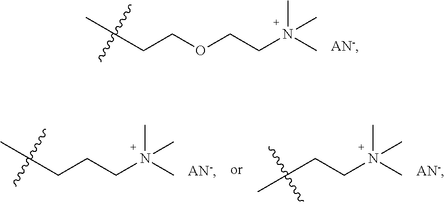

p is 1, 2 or 3;
R^{j} is

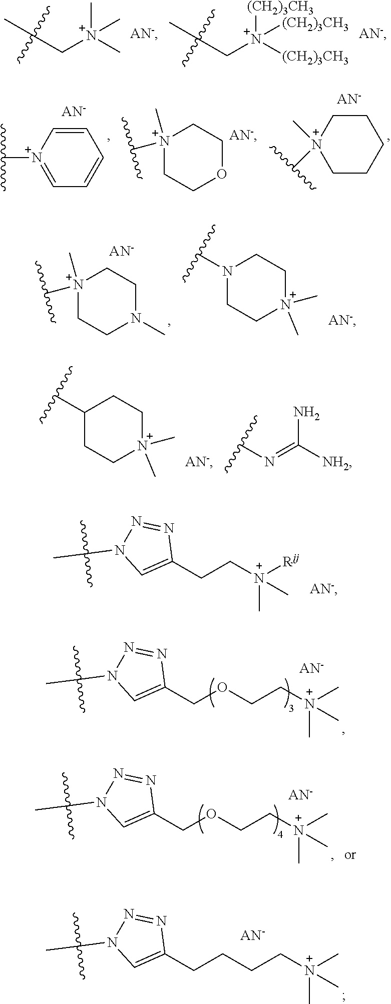

q is 1, 2, 3, 4, or 5;
R^{k} is —OCH₃,

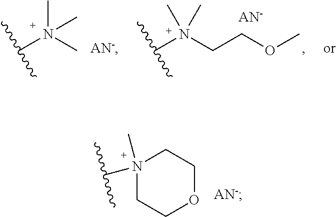

r is 3, 4, 5, 6, or 8;
wherein:
   R^{jj} is H, —(CH₂)₇CH₃, —(CH₂)₁₅CH₃, —(CH₂)₂OCH₃, or —(CH₂CH₂O)₃CH₃;
R¹ is —C(O)C₍₁₋₂₎alkyl, —C(O)CF₃, —COCH₂CF₃, —CO(CH₂)₅N(CH₃)₃⁺AN⁻, —CH₂CH₂NHC(O)CH₂O(CH₂CH₂O)₂CH₃, —CH₂CH₂NHC(O)(CH₂CH₂O)₆CH₃,

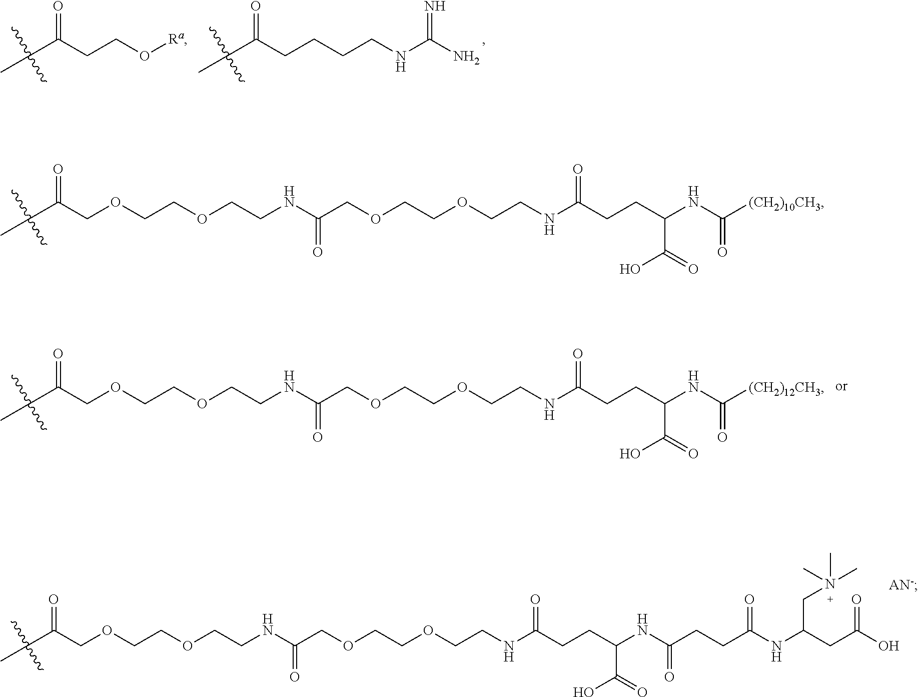

wherein:
   R^{a} is

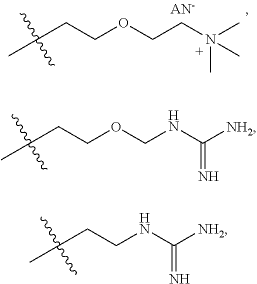

CH₂CH₂OCH₂CH₂N(CH₃)₂,
—(CH₂CH₂O)₃CH₂CH₂N(CH₃)₂, —(CH₂CH₂O)₃CH₂CH₂N(CH₃)₃ ⁺AN⁻, —(CH₂CH₂O)₅CH₂CH₂NC(O)CH₃, —(CH₂CH₂O)₈CH₂CH₂NC(O)CH₃, or —(CH₂CH₂O)₁₁CH₂CH₂NC(O)CH₃;
X3 is a direct bond, (D) arginine, (D) lysine substituted with R^{b},

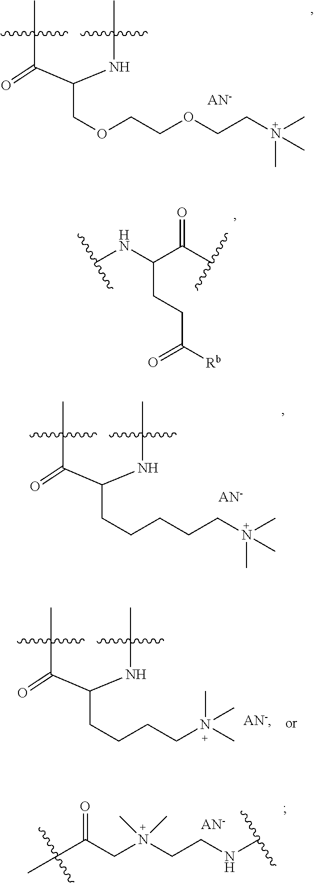

wherein:
H
   R^{b} is

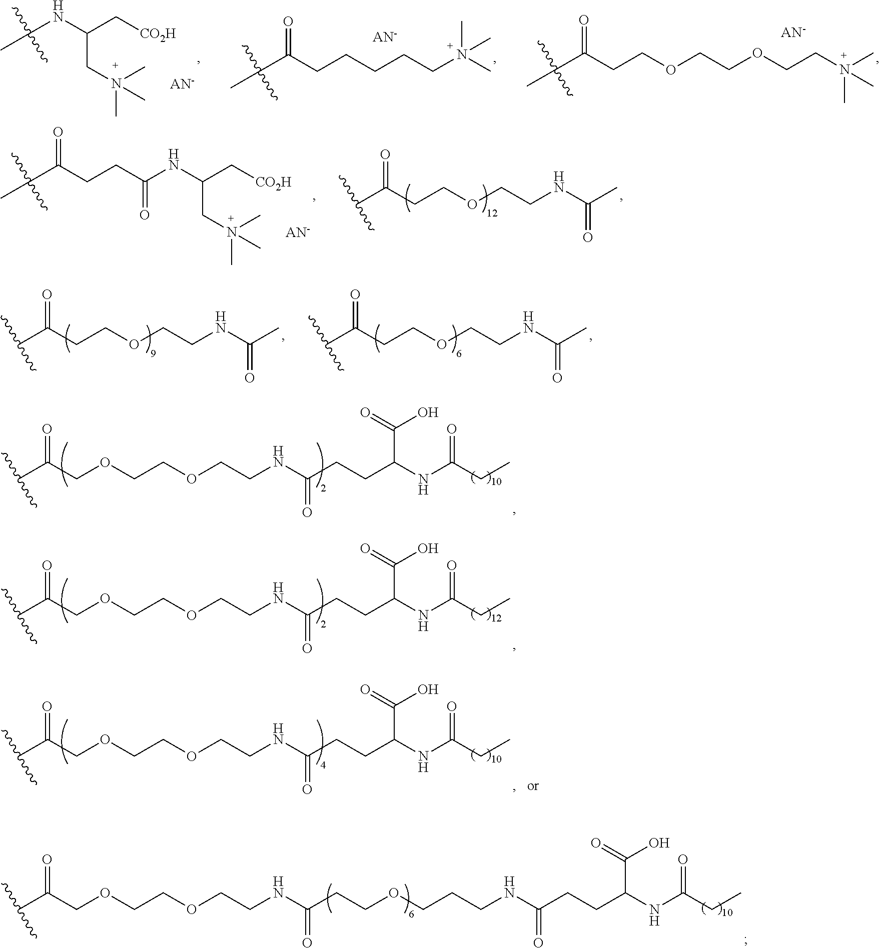

R² is H, —CH(CH₃)₂,

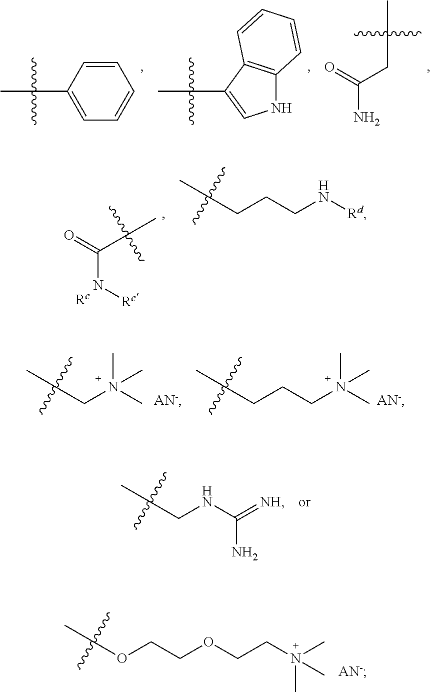

wherein:
   R^{c} is H, or —CH₃;
   R^{c}′ is H, or —CH₃;
R^{d} is

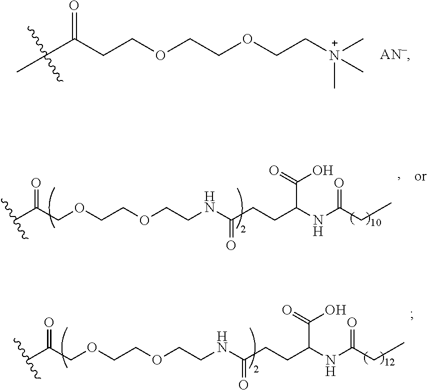

R³ is H, —CH₃, —CF₃, or

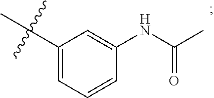 [SMILES: *c1cccc(NC(C)=O)c1]

R⁴ is H, or —CH₃;
X8 is

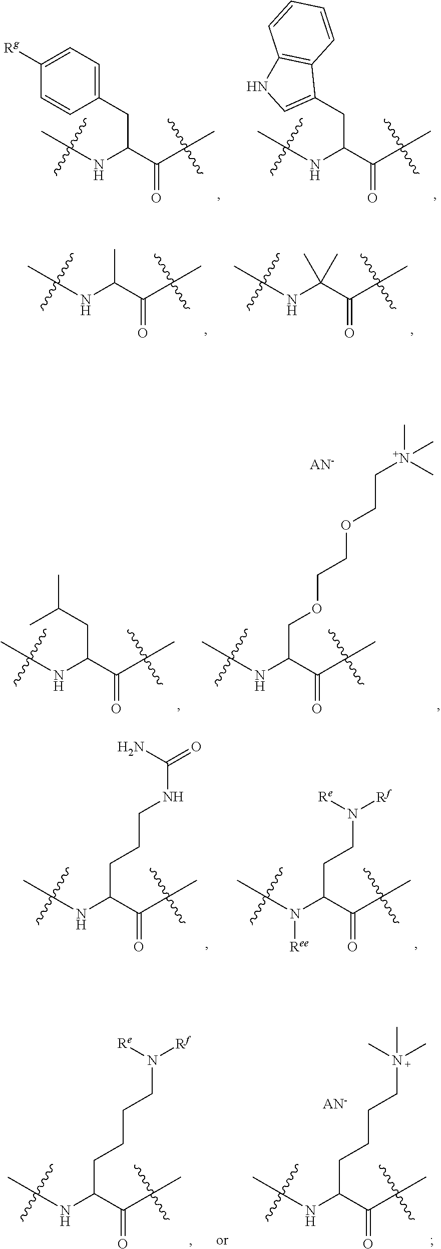

wherein:
   R^{e} is H, or —CH₃;
   R^{ee} is H;
   R^{f} is H, —CH₃, —C(CH₃)₃, —C(O)CH₃, —C(O)(CH₂CH₂O)₆CH₃, —(CH₂CH₂O)₆CH₃,

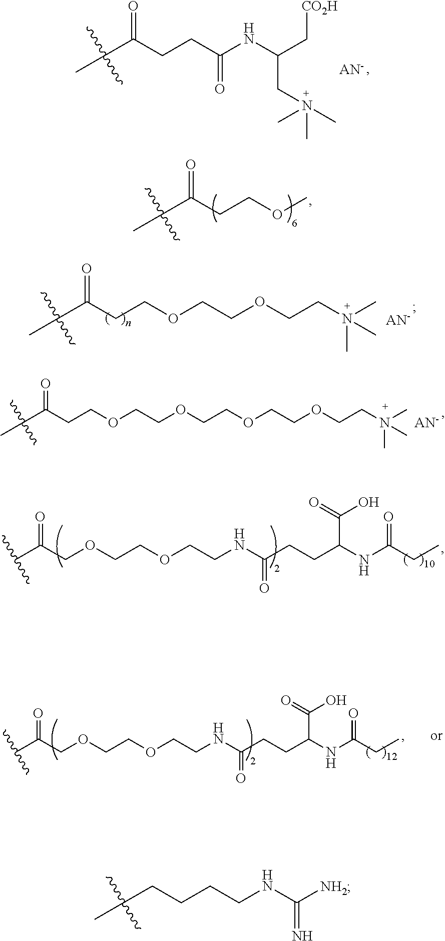

R^{g} is H, —OH, —C(O)NH₁₂, or —CH₂NHC(O)CH₃;
n is 0, or 1;
X12 is absent,

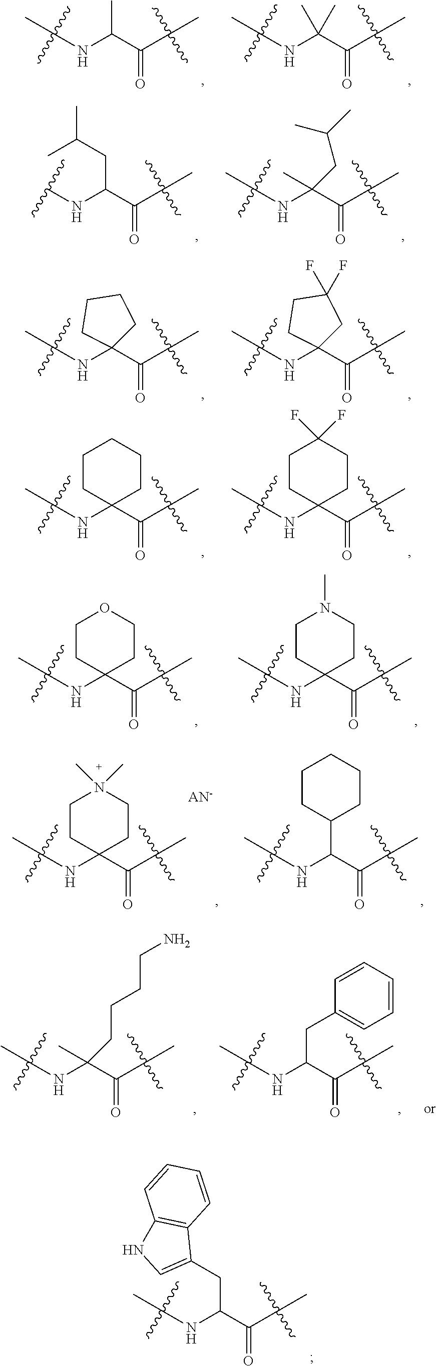

R⁶ is —CH₃, —CH₂CH₂C(O)R^{m}, —(CH₂)ₛNRⁿR^{o},

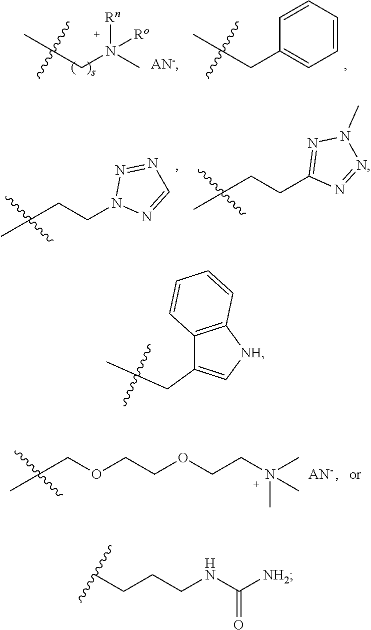

R⁷ is H, or —CH₃;
wherein:
   R^{m} is —OH, or;

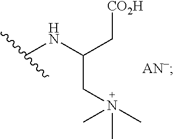

Rⁿ is H, —CH₃, —CH₂CH(CH₃)₂, —C(O)CH₃, —C(O)(CH₂CH₂O)₆CH₃, —(CH₂CH₂O)₆CH₃, —C(O)(CH₂CH₂O)ₜCH₂CH₂R^{p},

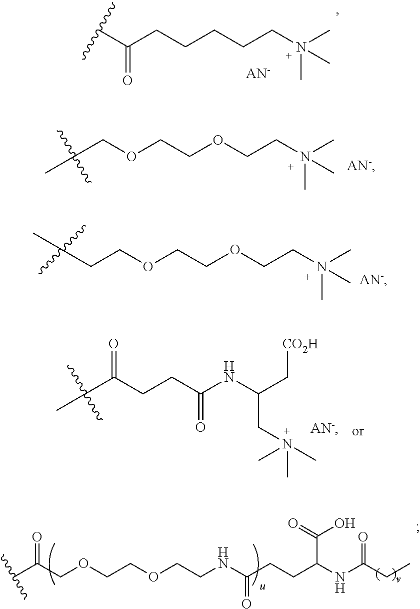

R^{o} is H, —CH₃, or —C(O)CH₃;
wherein:
   R^{p} is NHC(O)CH₃, or

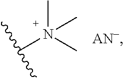

s is 1, 2, or 4;
t is 2, 6, 9, or 12;
u is 2 or 3;
v is 10 or 12;
R⁸ is —CH₂CH₂CH₂NHC(O)CH₃, —C(O)NH₂, or —C(O)NHCH₃;
X15 is absent,

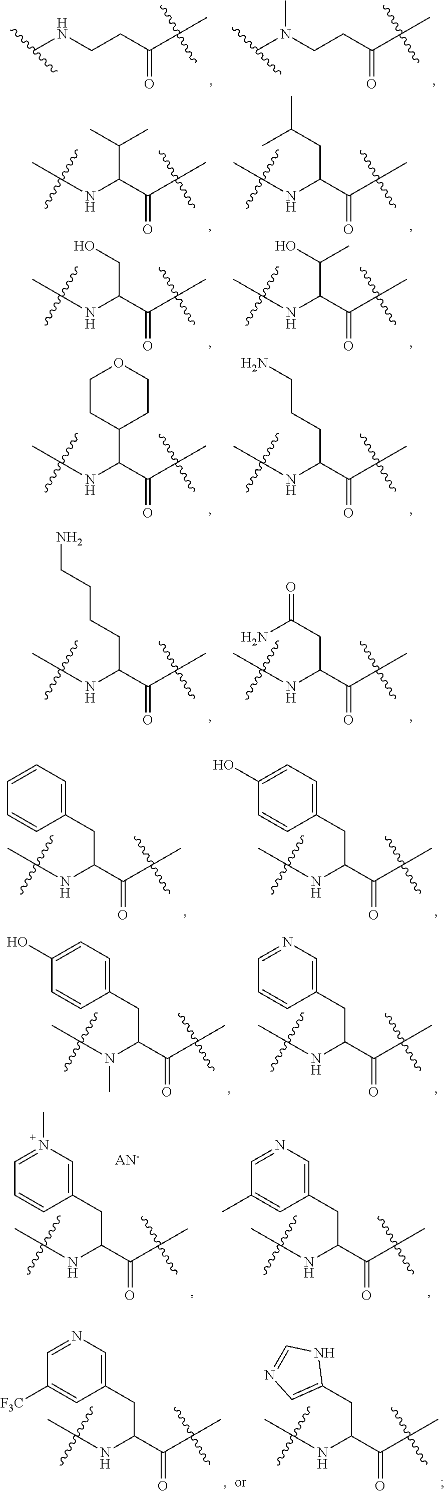

X16 is absent,

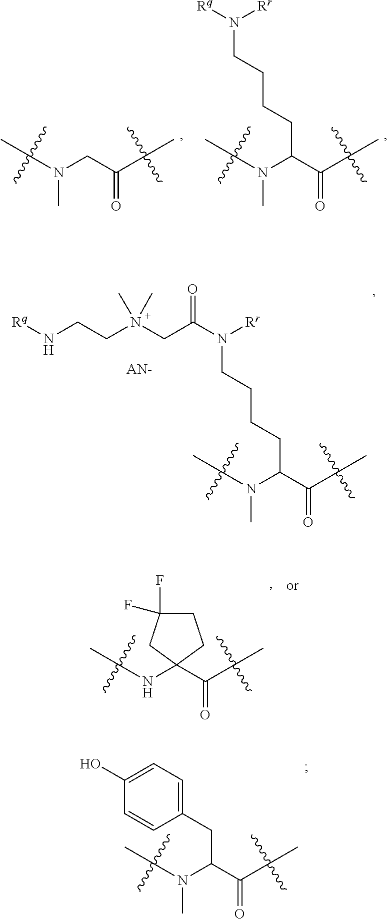

wherein:
   R^{q} is

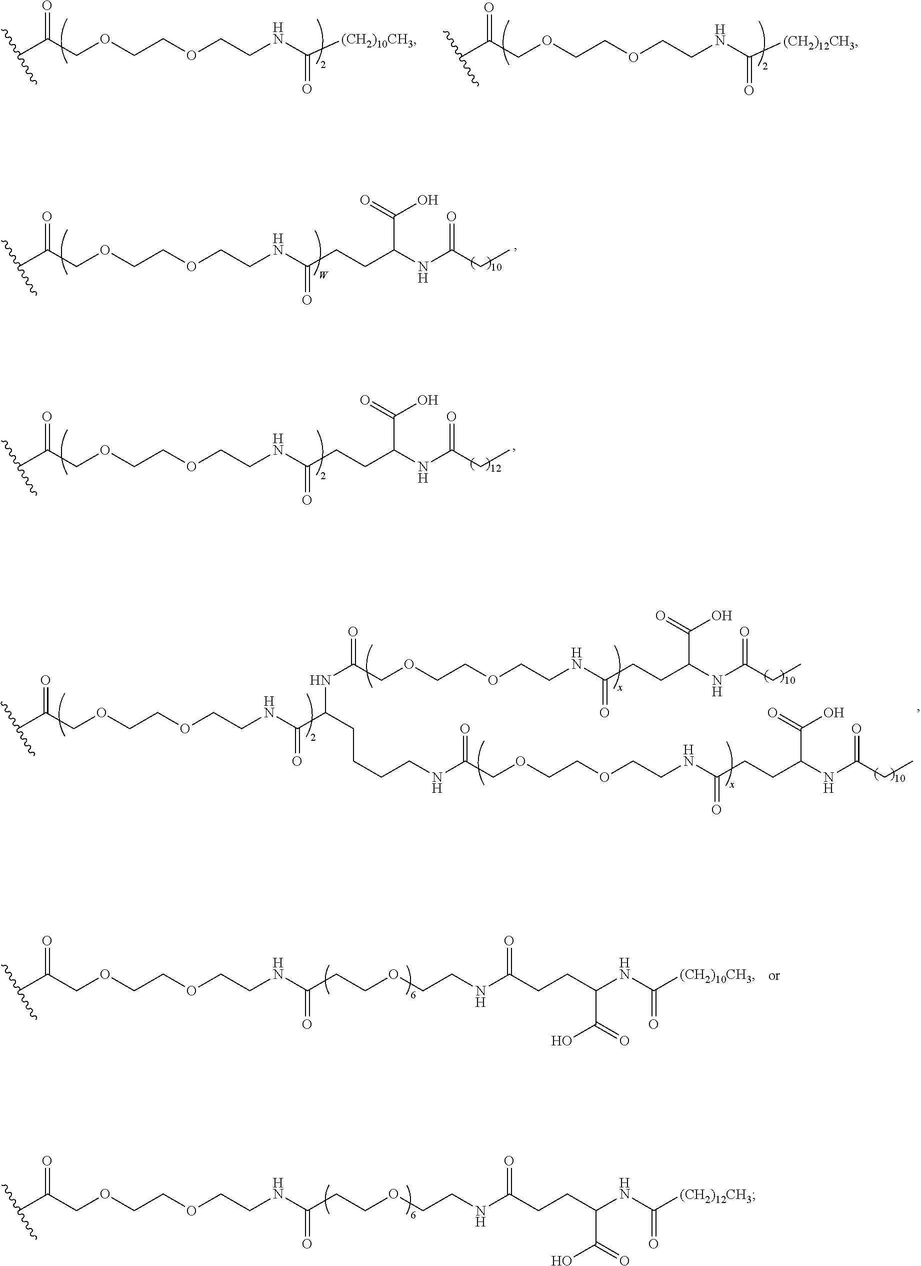

R^{r} is H;
w is 2 4, or 5;
x is 2 or 4; and
R⁹ is —NH₂, —NHCH₃, —N(CH₃)₂, —NH(CH₂CH₂O)₂CH₃, —N(CH₃)(CH₂CH₂O)₂CH₃, NH(CH₂CH₂O)_{y}R^{s},

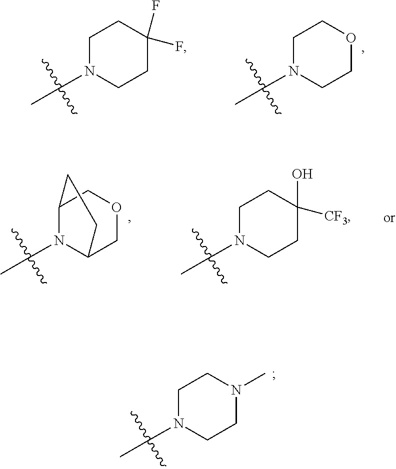

wherein:
   y is 2, 4, or 6;
   R^{s} is —CH₃, or

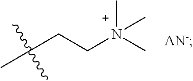 [SMILES: *CC[N+](C)(C)C]

wherein:
AN⁻as above defined for A, R, X3, X12, R1, R2, R5, R6, R8, R15, R16 and R^{a}, R^{b}, R^{d}, R^{f}, R^{h}, Rⁱ, R^{j}, R^{k}, R^{m}, Rⁿ, R^{o}, R^{s} is acetate, adipate, benzoate, benzenesulfonate, citrate, decanoate, chloride, lactate, maleate, methanesulfonate, oxalate, pivalate, propionate, succinate, sulfate, tartrate, or trifluoroacetate; or a pharmaceutically acceptable salt thereof.

### Clause 2.

2. A compound of Formula (II)

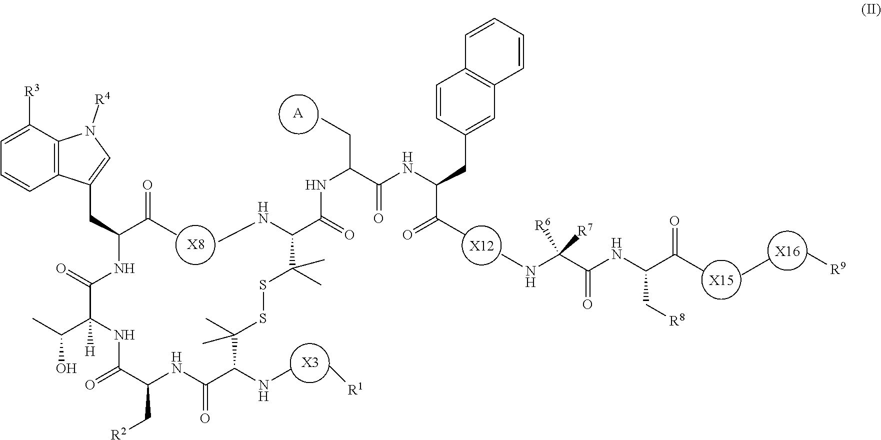

wherein
X12 is absent,

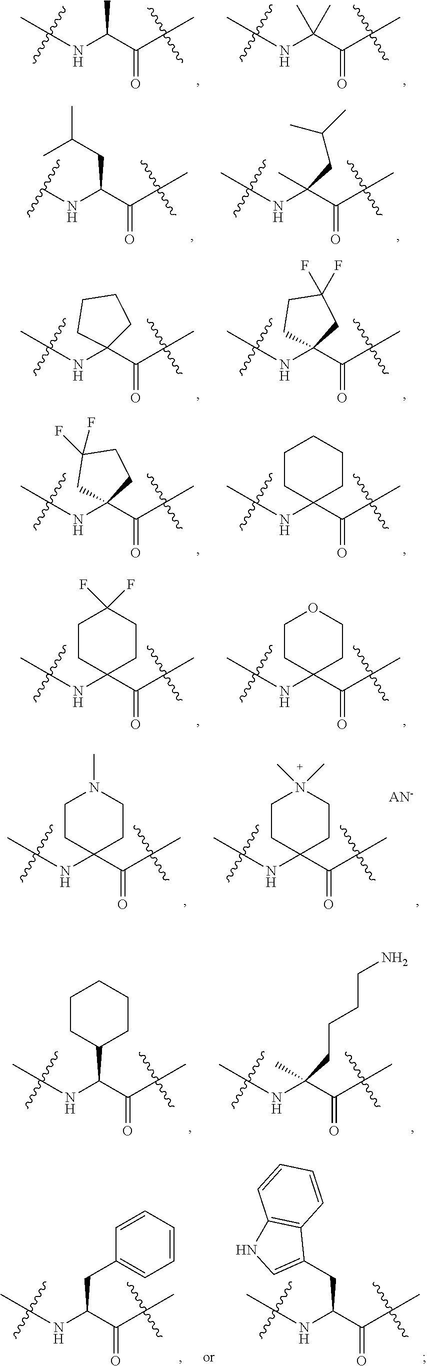

or
a pharmaceutically acceptable salt thereof.

### Clause 3

3. The compound of clause 2, wherein:
wherein:
A is

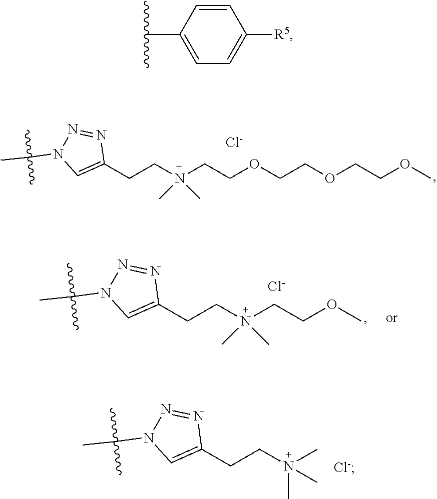

wherein:
   R⁵ is —OCHF₂, —O(CH₂)₉CO₂H,

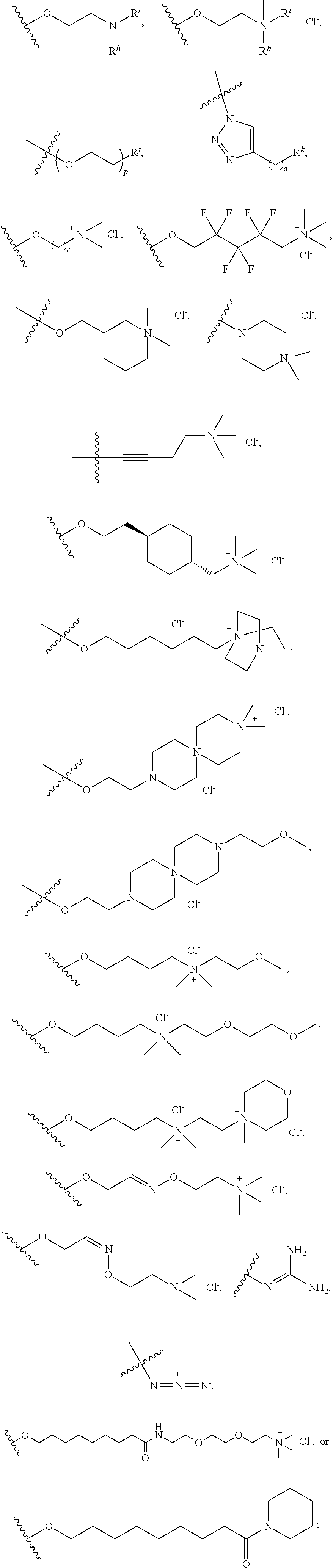

wherein:
   R^{h} is H, —CH₃, —C(O)CH₃, —C(NH)NH₂, —(CH₂)₃O(CH₂)₂OCH₃, —CH₂CH₂OCH₃, —(CH₂CH₂O)₃CH₃, —(CH₂CH₂O)₆CH₃,

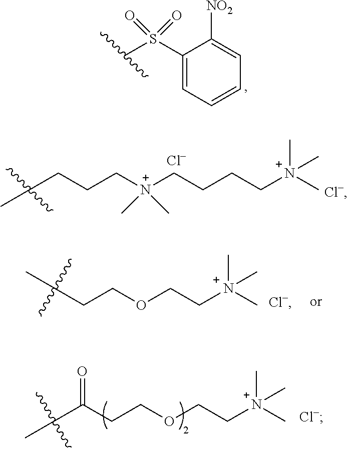

Rⁱ is H, —CH₃, —(CH₂)₃O(CH₂)₂OCH₃,

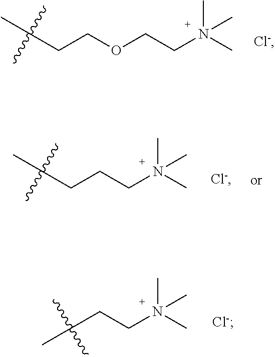

p is 1, 2 or 3;
R^{j} is

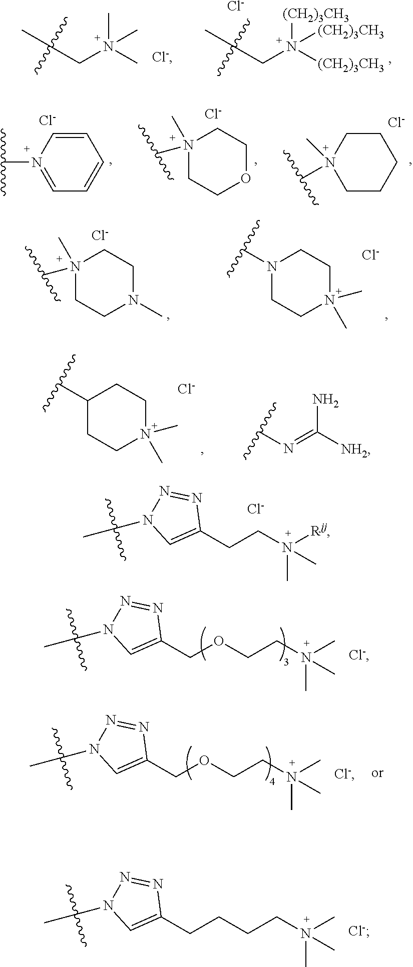

q is 1, 2, 3, 4, or 5;
R^{k} is —OCH₃,

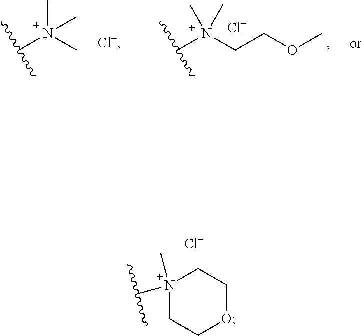

r is 3, 4, 5, 6, or 8;
wherein:
   R^{jj} is H, —(CH₂)₇CH₃, —(CH₂)₁₅CH₃, —(CH₂)₂OCH₃, or —(CH₂CH₂O)₃CH₃;
R¹ is —C(O)C₍₁₋₂₎alkyl, —C(O)CF₃, —COCH₂CF₃, —CO(CH₂)₅N(CH₃)₃⁺Cl⁻, —CH₂CH₂NHC(O)CH₂O(CH₂CH₂O)₂CH₃, —CH₂CH₂NHC(O)(CH₂CH₂O)₆CH₃,

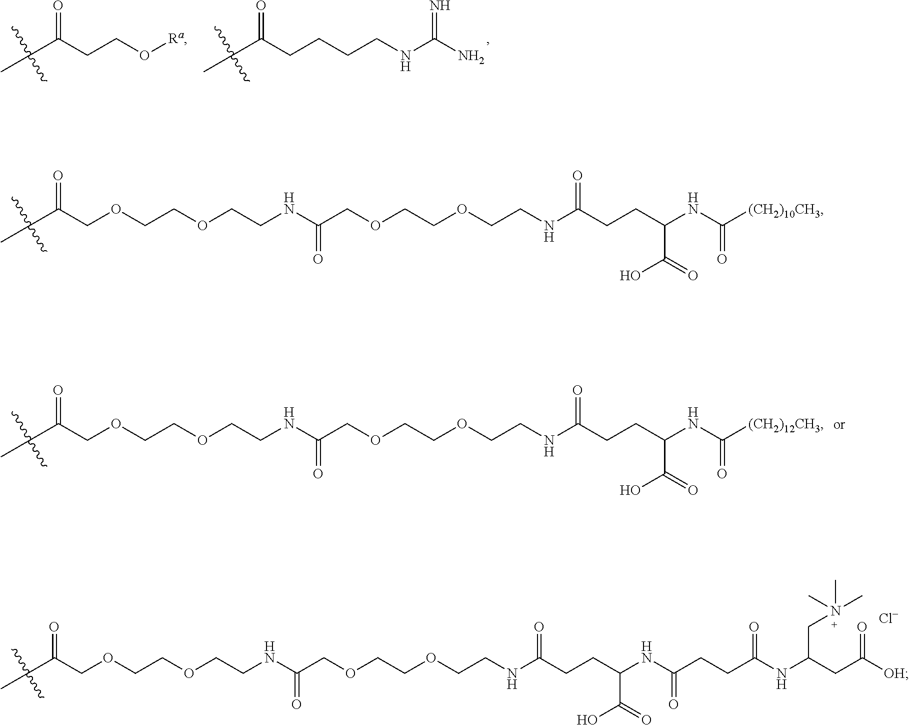

wherein:
   R^{a} is

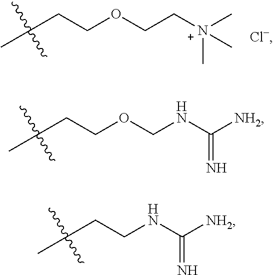

CH₂CH₂OCH₂CH₂N(CH₃)₂,
—(CH₂CH₂O)₃CH₂CH₂N(CH₃)₂, —(CH₂CH₂O)₃CH₂CH₂N(CH₃)₃⁺Cl⁻, —(CH₂CH₂O)₅CH₂CH₂NC(O)CH₃, —(CH₂CH₂O)₈CH₂CH₂NC(O)CH₃, or —(CH₂CH₂O)₁₁CH₂CH₂NC(O)CH₃;
X3 is a direct bond, (D) arginine, (D) lysine substituted with R^{b},

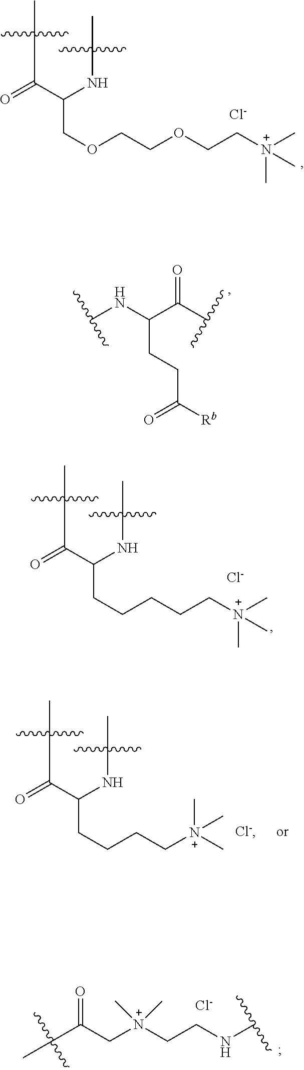

wherein:

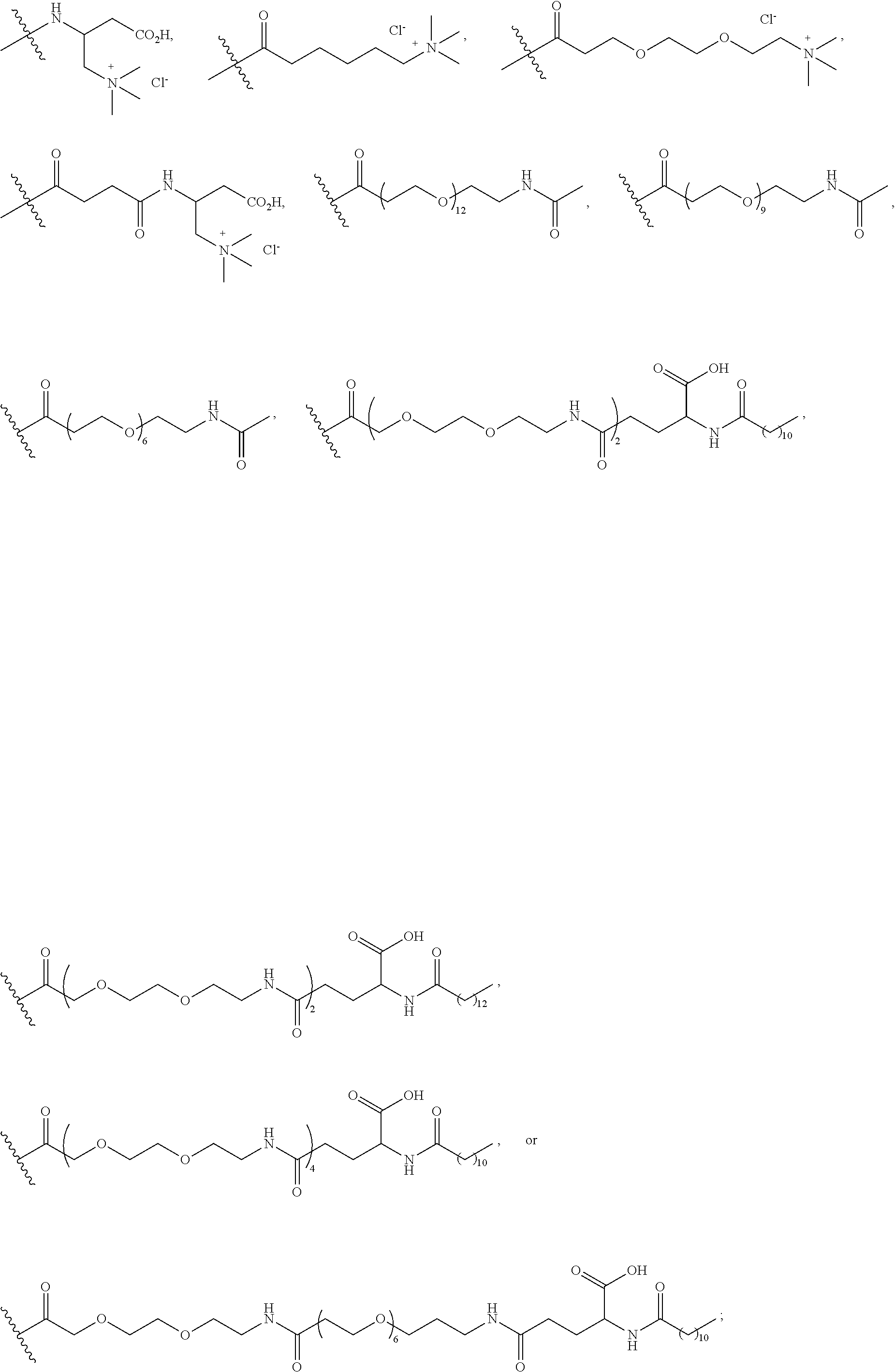

R² is H, —CH(CH₃)₂,

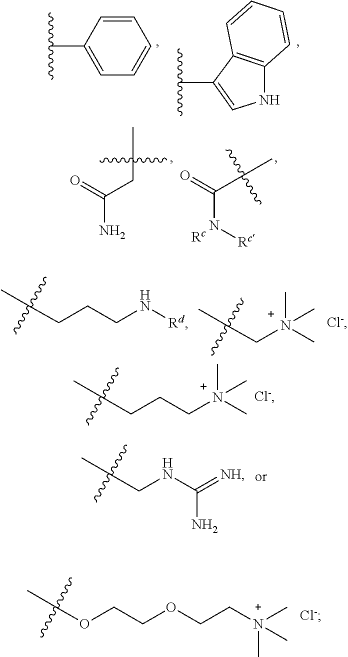

wherein:
   R^{c} is H, or —CH₃
   R^{c}′ is H, or —CH₃;
   R^{d} is

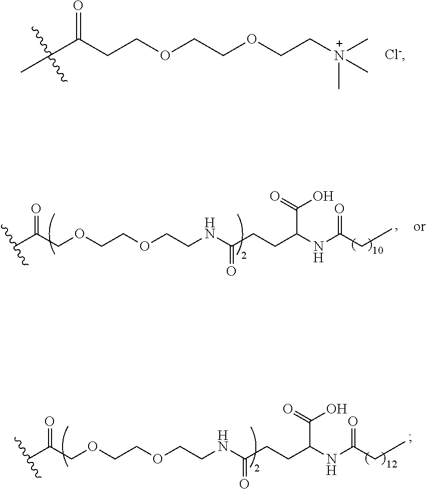

R³ is H, —CH₃, —CF₃, or

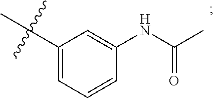 [SMILES: *c1cccc(NC(C)=O)c1]

R⁴ is H, or —CH₃;
X8 is,

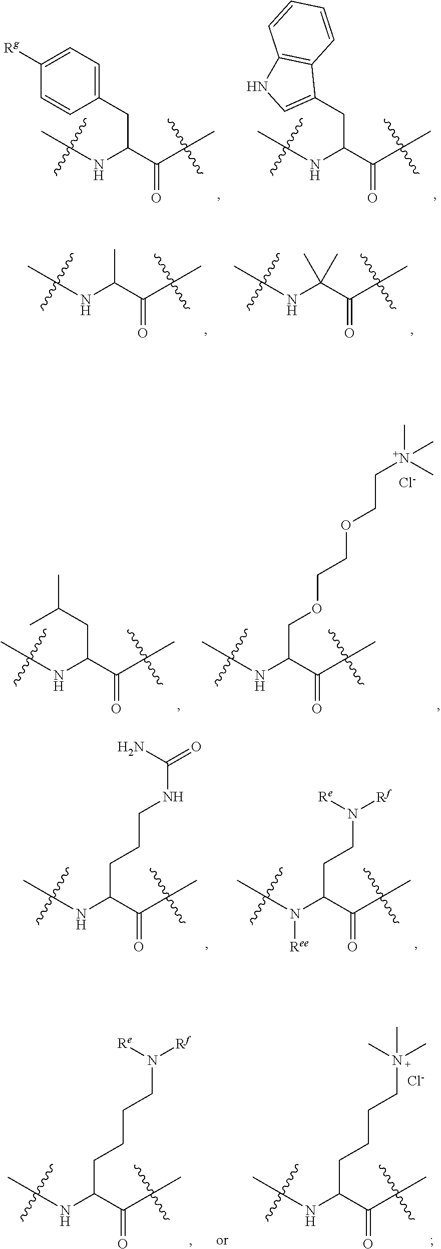

wherein:
   R^{e} is H, or —CH₃;
   R^{ee} is H;
   R^{f} is H, —CH₃, —C(CH₃)₃, —C(O)CH₃, —C(O)(CH₂CH₂O)₆CH₃, —(CH₂CH₂O)₆CH₃,

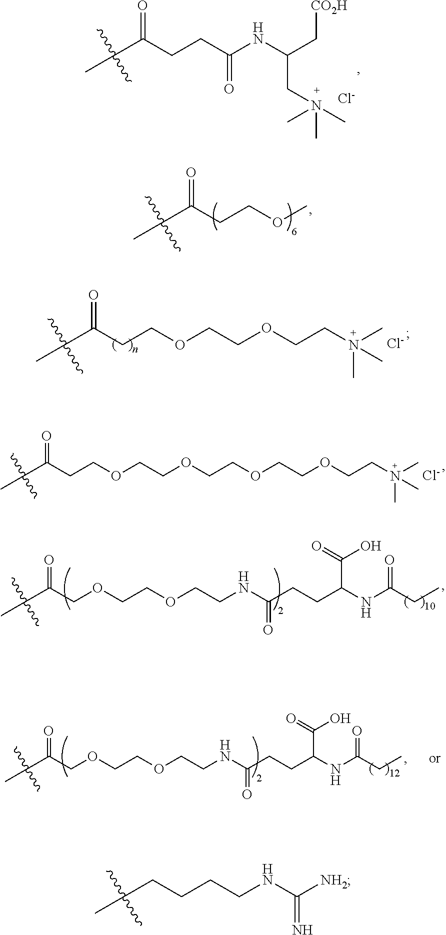

R^{g} is H, —OH, —C(O)NH₁₂, or —CH₂NHC(O)CH₃;
n is 0, or 1;
X12 is absent,

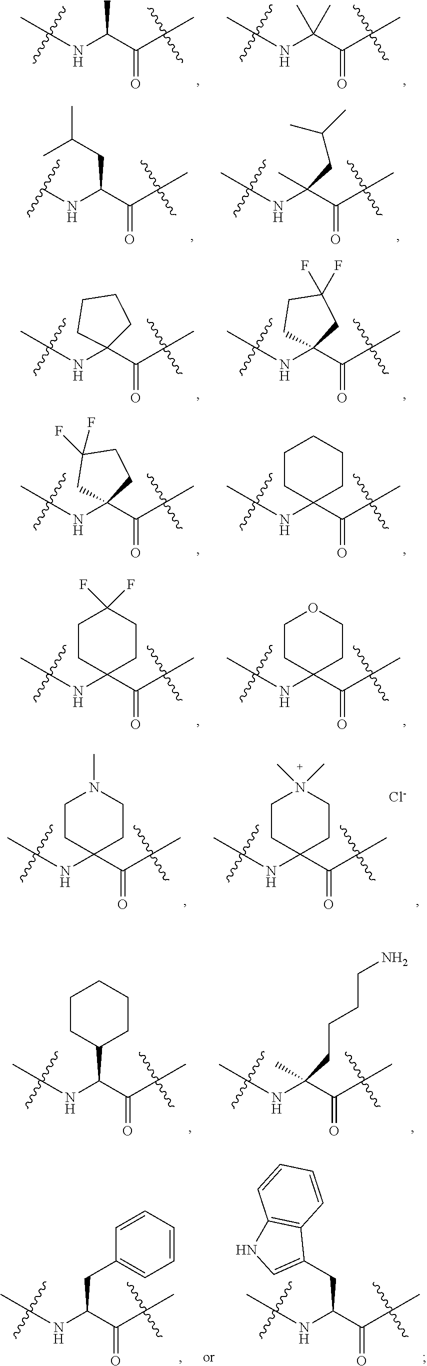

R⁶ is —CH₃, —CH₂CH₂C(O)R^{m}, —(CH₂)ₛNRⁿR^{o},

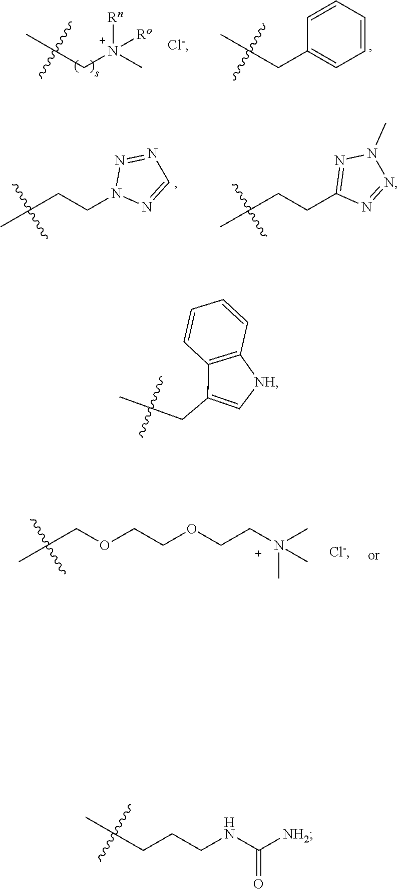

R⁷ is H, or —CH₃
wherein:
   R^{m} is —OH, or

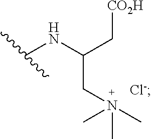

Rⁿ is H, —CH₃, —CH₂CH(CH₃)₂, —C(O)CH₃, —C(O)(CH₂CH₂O)₆CH₃, —(CH₂CH₂O)₆CH₃, —C(O)(CH₂CH₂O)ₜCH₂CH₂R^{p},

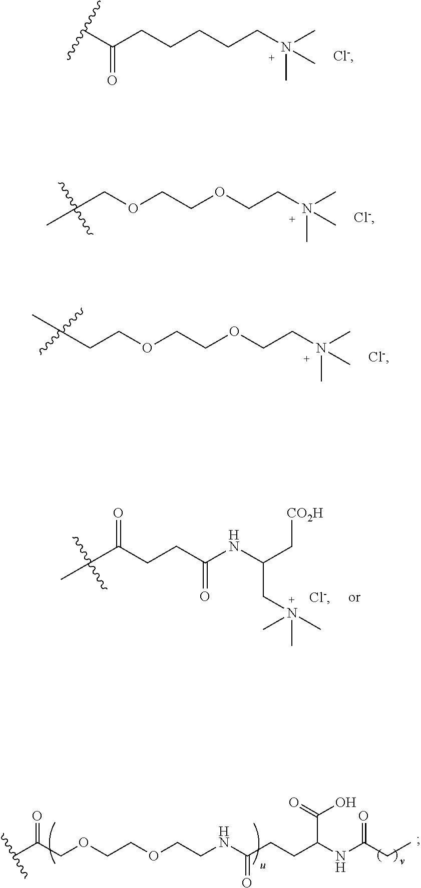

R^{o} is H, —CH₃, or —C(O)CH₃;
wherein:
   R^{p} is NHC(O)CH₃, or

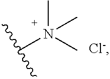

s is 1, 2, or 4;
t is 2, 6, 9, or 12;
u is 2 or 3;
v is 10 or 12;
R⁸ is —CH₂CH₂CH₂NHC(O)CH₃, —C(O)NH₂, or —C(O)NHCH₃;
X15 is absent,

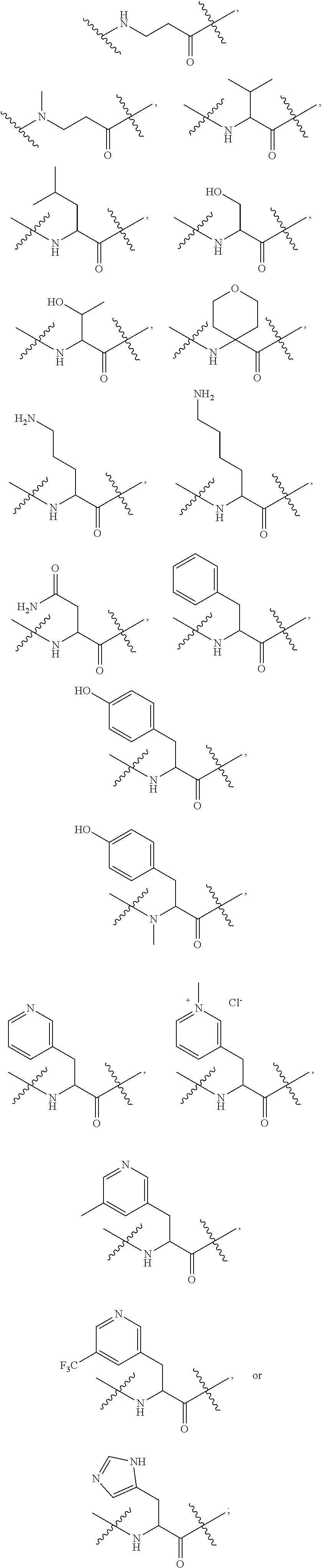

X16 is absent,

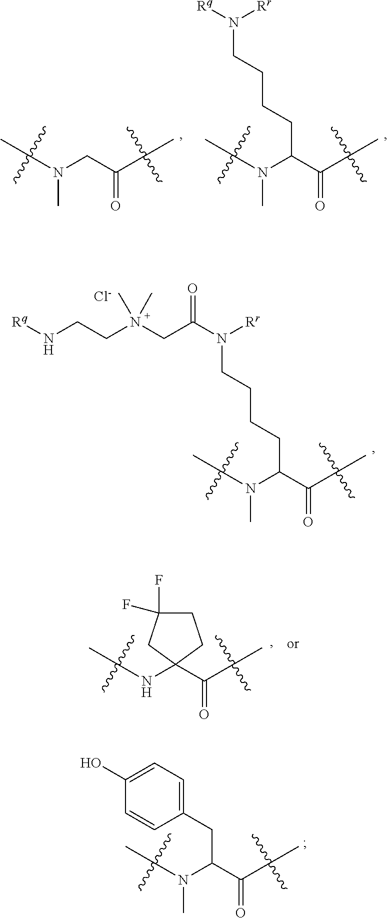

wherein:
   R^{q} is

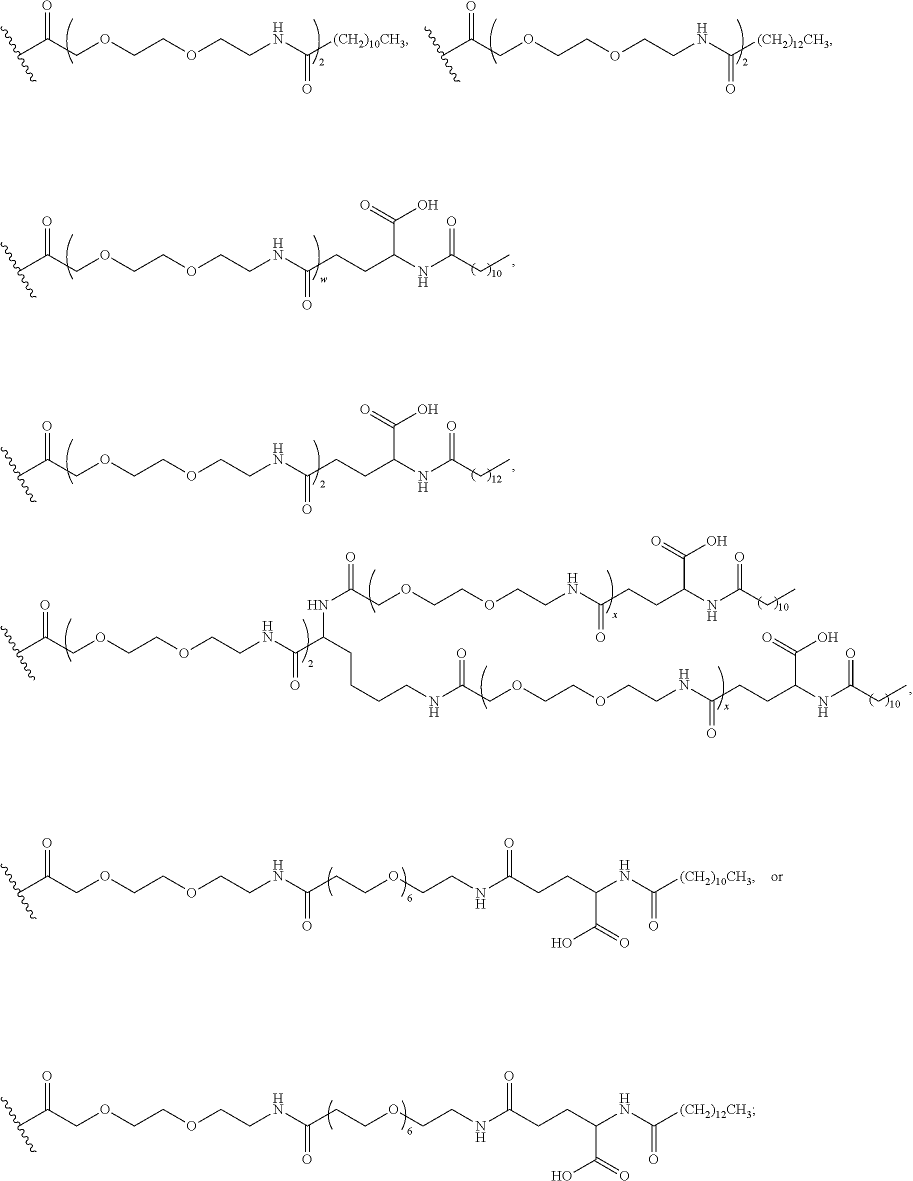

R^{r} is H;
w is 24, or 5;
x is 20r 4; and
R⁹ is —NH₂, —NHCH₃, —N(CH₃)₂, —NH(CH₂CH₂O)₂CH₃, —N(CH₃)(CH₂CH₂O)₂CH₃, NH(CH₂CH₂O)_{y}R^{s}, 1, or

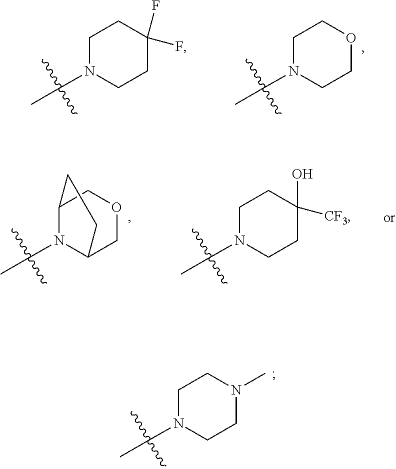

wherein:
   y is 2, 4, or 6;
   R^{s} is —CH₃, or

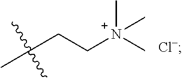

or
a pharmaceutically acceptable salt thereof.

### Clause 4

4. The compound of any of clauses 1 to 3 wherein
R² is

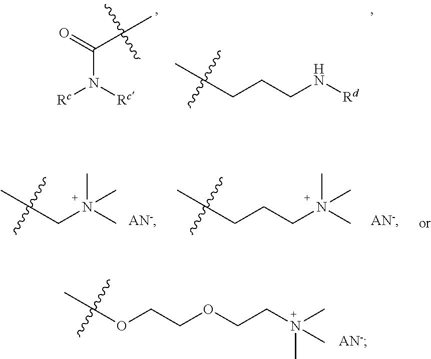

wherein:
   R^{c} is —CH₃;
   R^{c}′ is —CH₃;
   R^{d} is

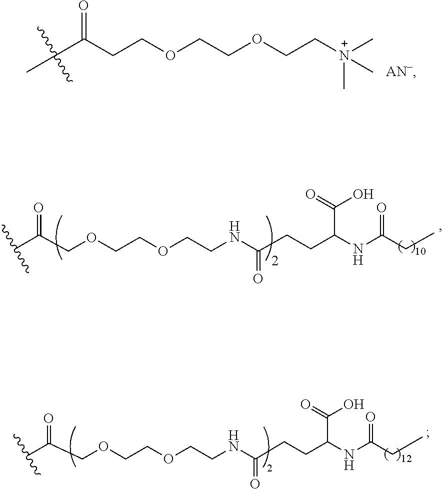

wherein:
AN⁻as above defined for A, R, X3, X12, R1, R2, R5, R6, R8, R15, R16 and R^{a}, R^{b}, R^{d}, R^{f}, R^{h}, Rⁱ, R^{j}, R^{k}, R^{m}, Rⁿ, R^{o}, R^{s} is acetate, adipate, benzoate, benzenesulfonate, citrate, decanoate, chloride, lactate, maleate, methanesulfonate, oxalate, pivalate, propionate, succinate, sulfate, tartrate, or trifluoroacetate; or
a pharmaceutically acceptable salt thereof.

### Clause 5

5. A compound according to Formula (III)

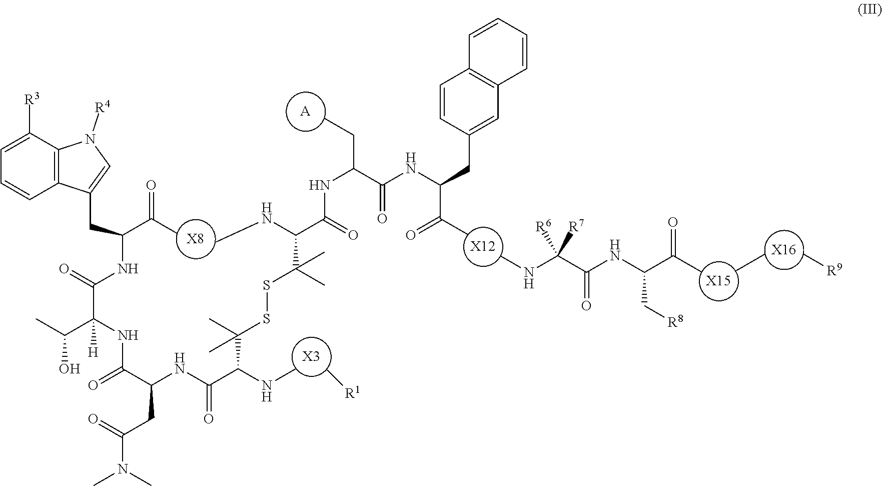

wherein:
A is

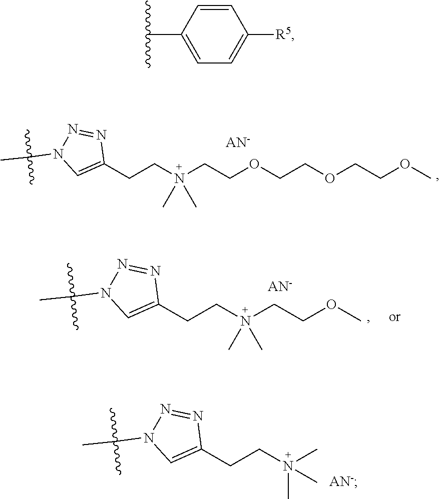

wherein:

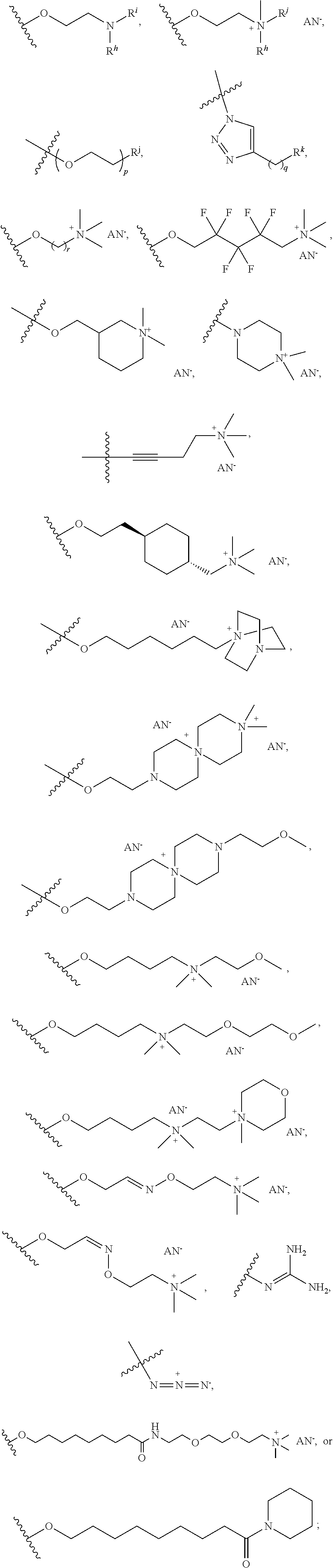

R⁵ is —OCHF₂, —O(CH₂)₉CO₂H,
wherein:
   R^{h} is H, —CH₃, —C(O)CH₃, —C(NH)NH₂, —(CH₂)₃O(CH₂)₂OCH₃, —CH₂CH₂OCH₃, —(CH₂CH₂O)₃CH₃, —(CH₂CH₂O)₆CH₃,

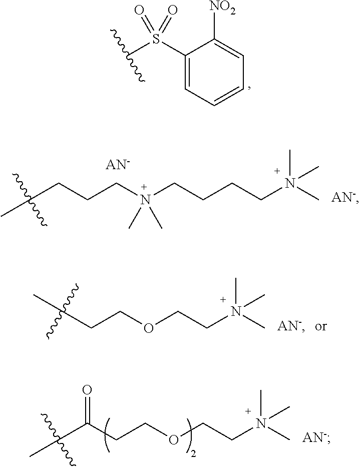

Rⁱ is H, —CH₃, —(CH₂)₃O(CH₂)₂OCH₃,

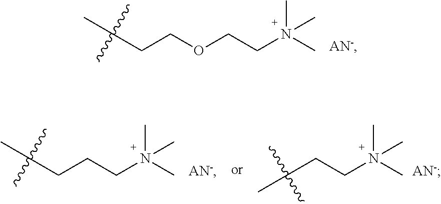

p is 1, 2 or 3;
R^{j} is

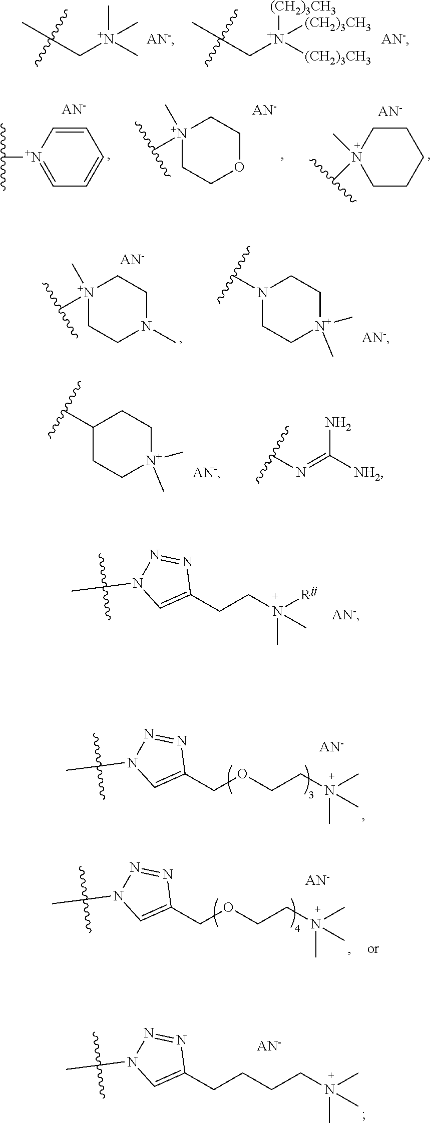

q is 1, 2, 3, 4, or 5;
R^{k} is —OCH₃,

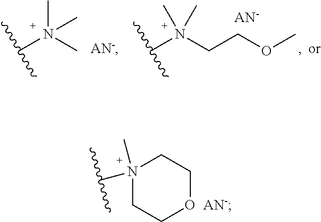

r is 3, 4, 5, 6, or 8;
wherein:
   R^{jj} is H, —(CH₂)₇CH₃, —(CH₂)₁₅CH₃, —(CH₂)₂OCH₃, or —(CH₂CH₂O)₃CH₃;
R¹ is —C(O)C₍₁₋₂₎alkyl, —C(O)CF₃, —COCH₂CF₃, —CO(CH₂)₅N(CH₃)₃⁺AN⁻, —CH₂CH₂NHC(O)CH₂O(CH₂CH₂O)₂CH₃, —CH₂CH₂NHC(O)(CH₂CH₂O)₆CH₃,

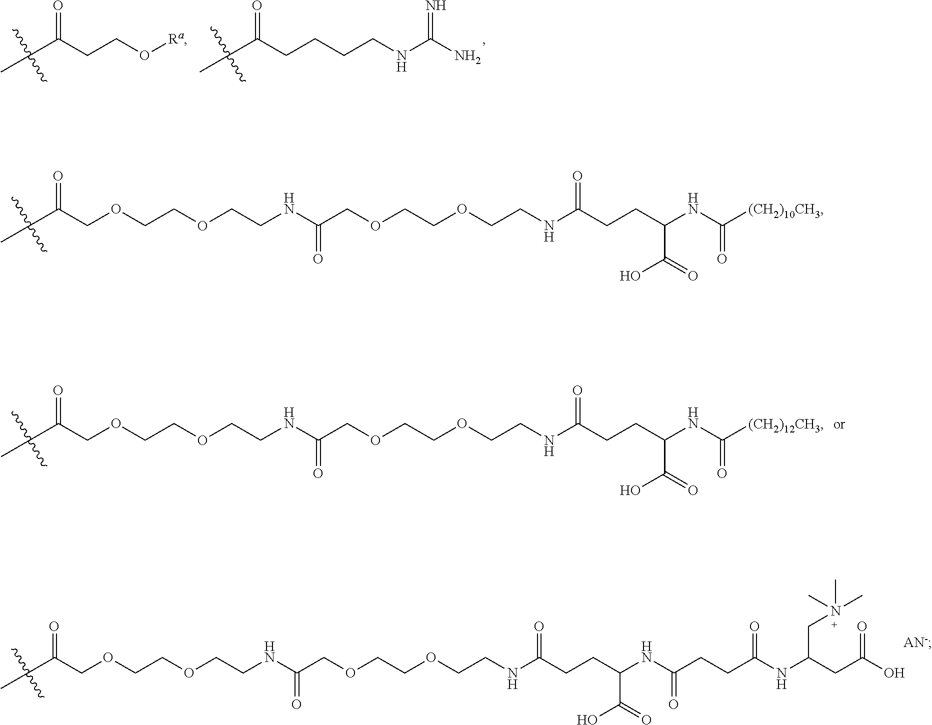

wherein:
   R^{a} is

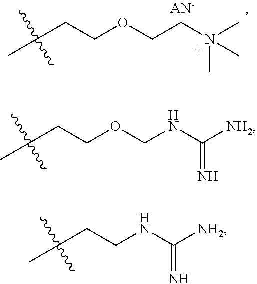

CH₂CH₂OCH₂CH₂N(CH₃)₂,
—(CH₂CH₂O)₃CH₂CH₂N(CH₃)₂, —(CH₂CH₂O)₃CH₂CH₂N(CH₃)₃ ⁺AN⁻, —(CH₂CH₂O)₅CH₂CH₂NC(O)CH₃, —(CH₂CH₂O)₈CH₂CH₂NC(O)CH₃, or —(CH₂CH₂O)₁₁CH₂CH₂NC(O)CH₃;
X3 is a direct bond, (D) arginine, (D) lysine substituted with R^{b},

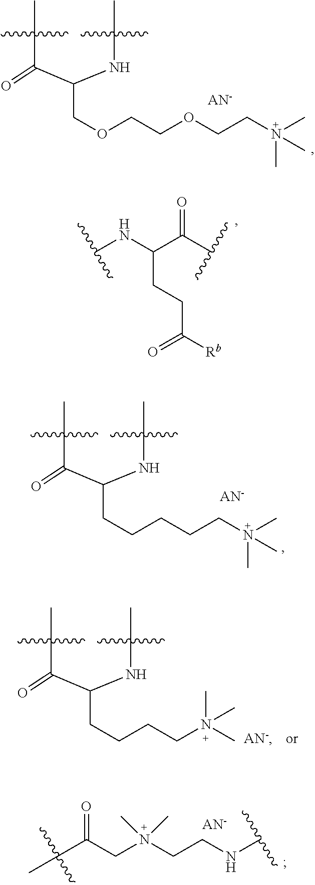

wherein:
   R^{b} is

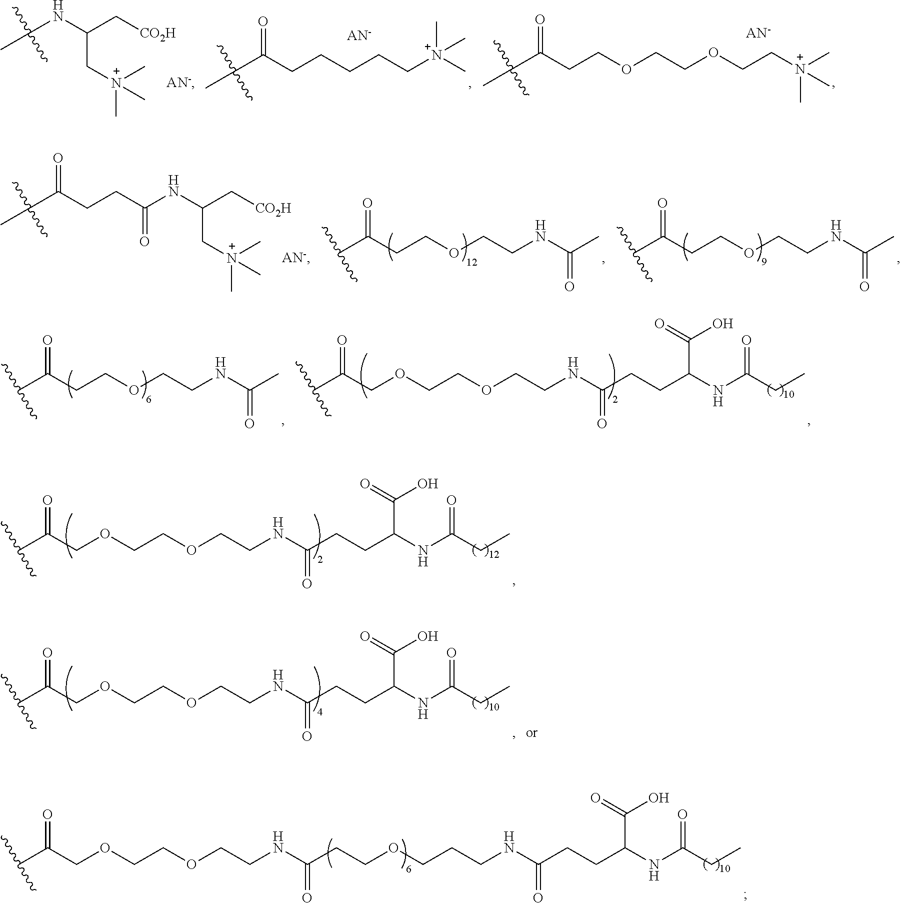

R³ is H, —CH₃, —CF₃, or

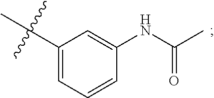 [SMILES: *c1cccc(NC(C)=O)c1]

R⁴ is H, or —CH₃;
X8 is

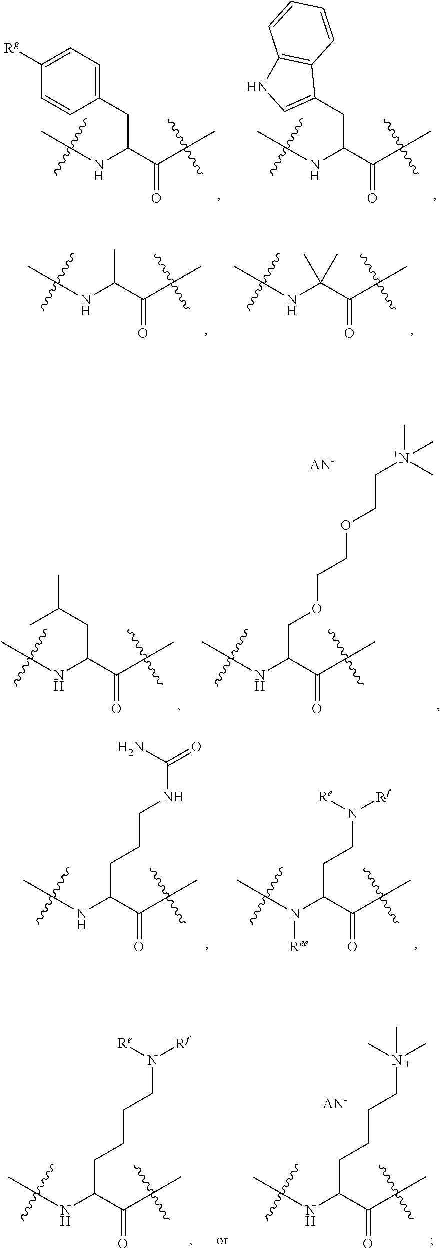

wherein:
   R^{e} is H, or —CH₃;
   R^{ee} is H;
   R^{f} is H, —CH₃, —C(CH₃)₃, —C(O)CH₃, —C(O)(CH₂CH₂O)₆CH₃,

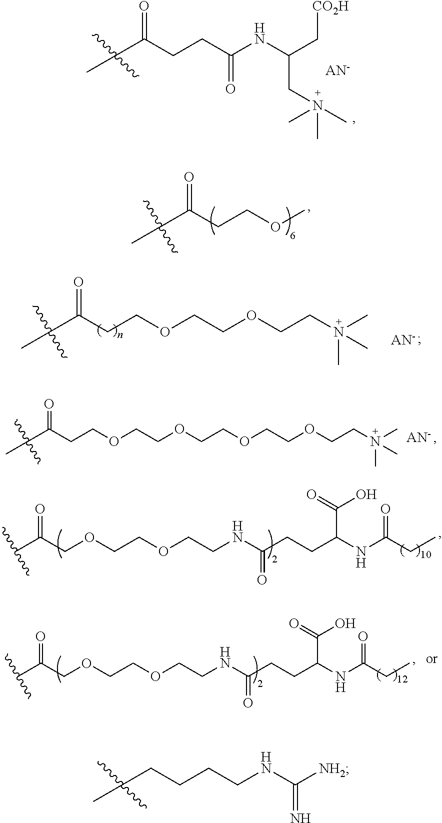

R^{g} is H, —OH, —C(O)NH₂, or —CH₂NHC(O)CH₃;
n is 0, or 1;
X12 is absent

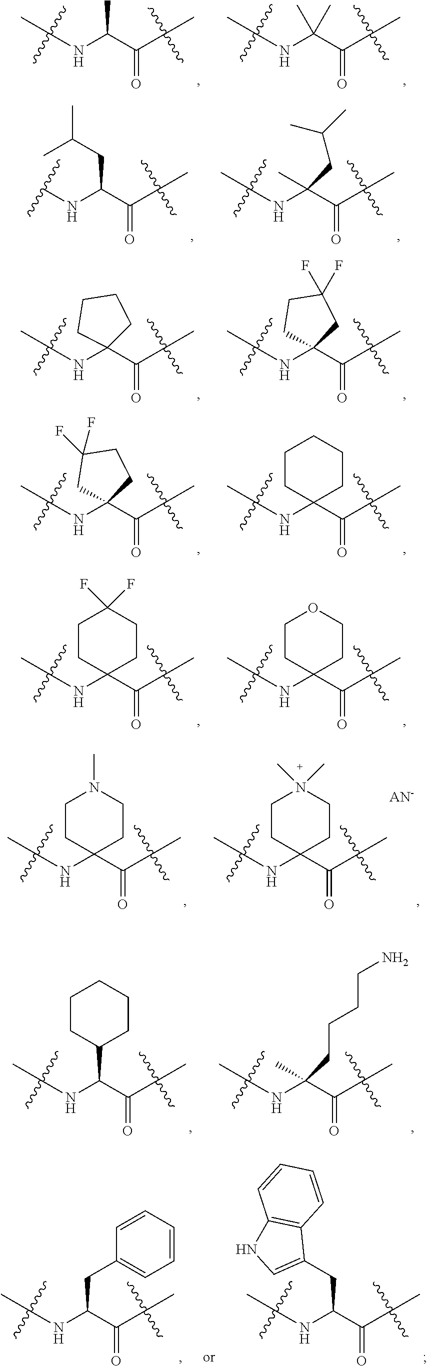

R⁶ is —CH₃, —CH₂CH₂C(O)R, —(CH₂)ₛNRⁿR^{o},

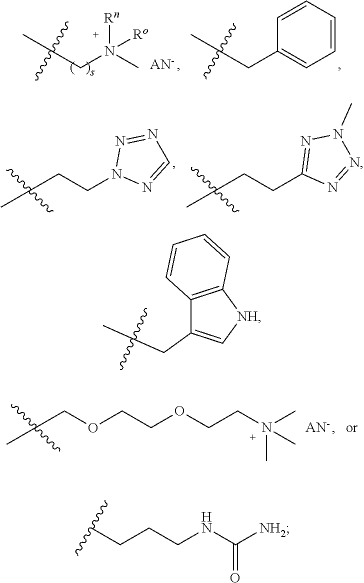

R⁷ is H, or —CH₃;
wherein:
   R^{m} is —OH, or

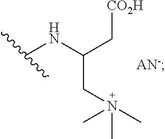

Rⁿ is H, —CH₃, —CH₂CH(CH₃)₂, —C(O)CH₃, —C(O)(CH₂CH₂O)₆CH₃, —(CH₂CH₂O)₆CH₃, —C(O)(CH₂CH₂O)ₜCH₂CH₂R^{p},

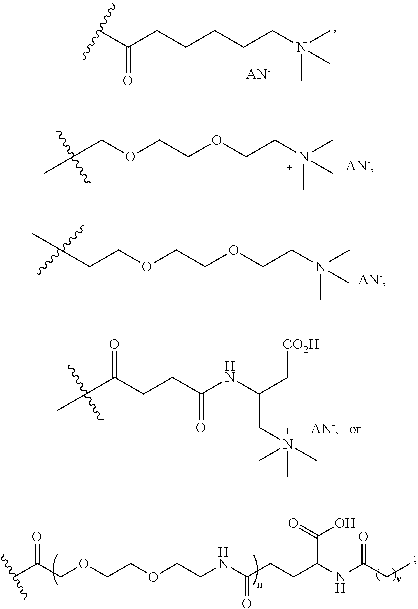

R^{o} is H, —CH₃, or —C(O)CH₃;
wherein:
   R^{p} is NHC(O)CH₃, or

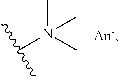

s is 1,2, or 4;
t is 2, 6, 9, or 12;
u is 2 or 3;
v is 10 or 12;
R⁸ is —CH₂CH₂CH₂NHC(O)CH₃, —C(O)NH₂, or —C(O)NHCH₃;
X15 is absent,

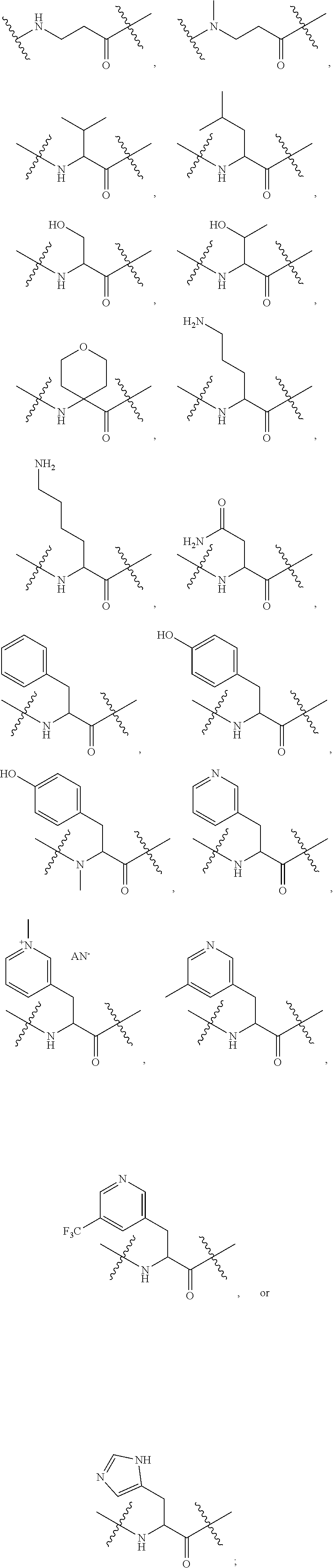

X16 is absent,

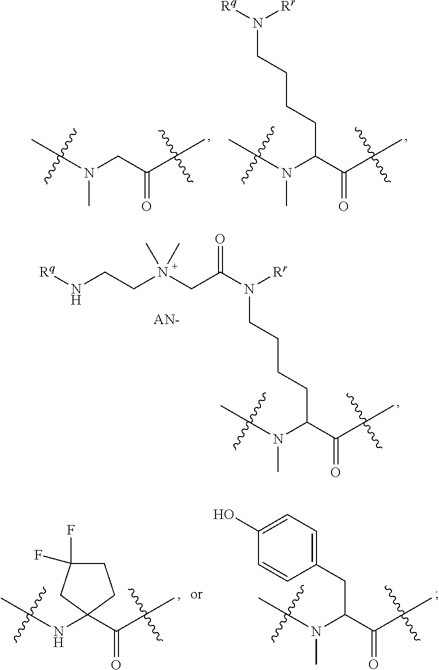

wherein:
   R^{q} is

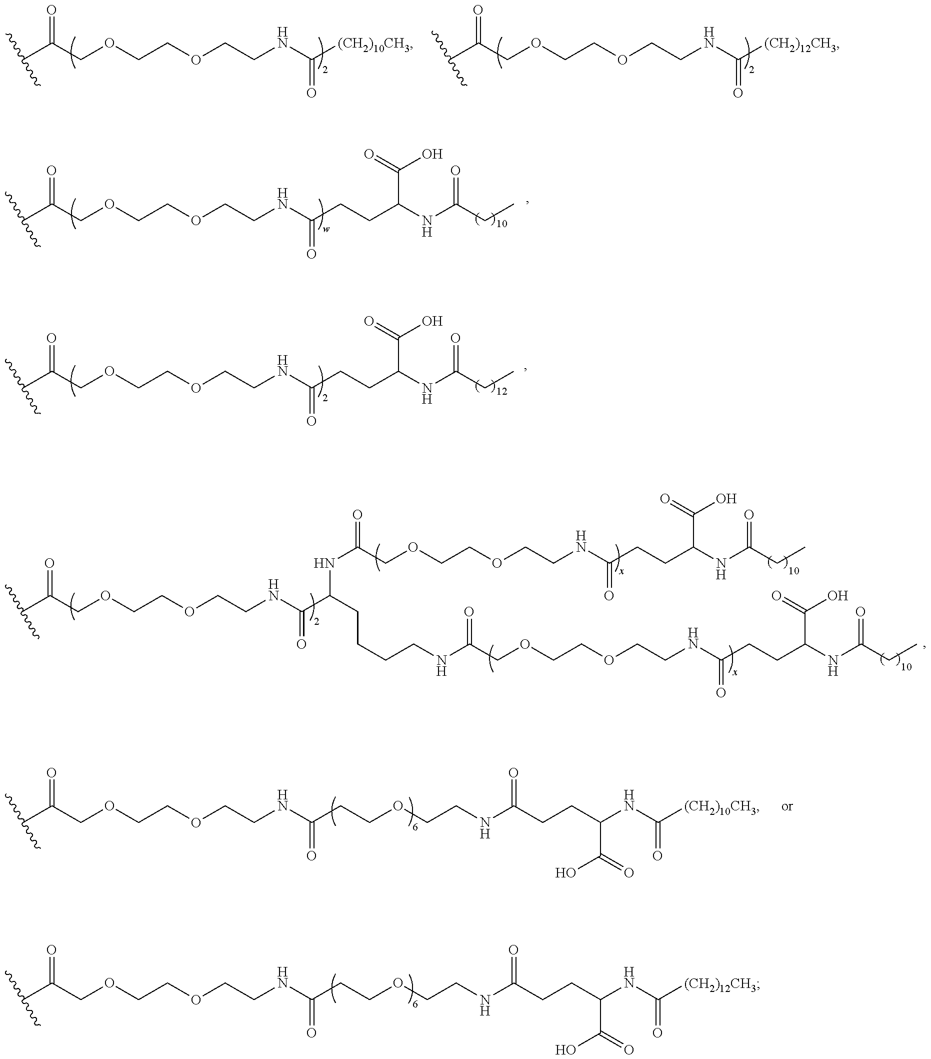

R^{r} is H;
w is 2 4, or 5;
x is 2 or 4; and
R⁹ is —NH₂, —NHCH₃, —N(CH₃)₂, —NH(CH₂CH₂O)₂CH₃, —N(CH₃)(CH₂CH₂O)₂CH₃, —NH(CH₂CH₂O)_{y}R^{s},

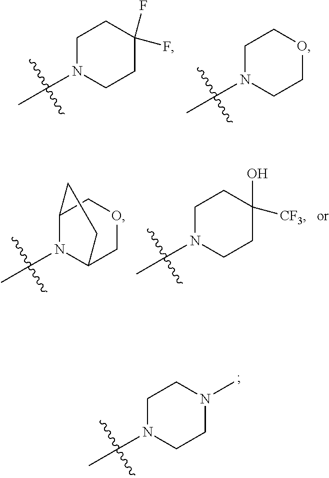

wherein:
   y is 2, 4, or 6;
   R^{s} is —CH₃, or

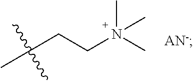

wherein:
AN⁻as above defined for A, R, X3, X12, R1, R2, R5, R6, R8, R15, R16 and R^{a}, R^{b}, R^{d}, R^{f}, R^{h}, Rⁱ, R^{j}, R^{k}, R^{m}, Rⁿ, R^{o}, R^{s} is acetate, adipate, benzoate, benzenesulfonate, citrate, decanoate, chloride, lactate, maleate, methanesulfonate, oxalate, pivalate, propionate, succinate, sulfate, tartrate, or trifluoroacetate; or
a pharmaceutically acceptable salt thereof.

### Clause 6

6. The compound of clause 5 wherein
A is

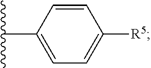

wherein:
   AN⁻as above defined for A, R, X3, X12, R1, R2, R5, R6, R8, R15, R16 and R^{a}, R^{b}, R^{d}, R^{f}, R^{h}, Rⁱ, R^{j}, R^{k}, R^{m}, Rⁿ, R^{o}, R^{s} is acetate, adipate, benzoate, benzenesulfonate, citrate, decanoate, chloride, lactate, maleate, methanesulfonate, oxalate, pivalate, propionate, succinate, sulfate, tartrate, or trifluoroacetate; or
a pharmaceutically acceptable salt thereof.

### Clause 7

7. The compound of clause 5 wherein
A is

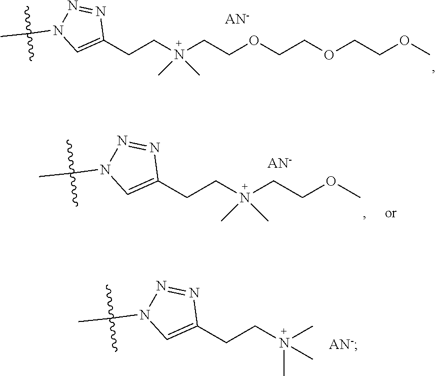

wherein:
   AN⁻as above defined for A, R, X3, X12, R1, R2, R5, R6, R8, R15, R16 and R^{a}, R^{b}, R^{d}, R^{f}, R^{h}, Rⁱ, R^{j}, R^{k}, R^{m}, Rⁿ, R^{o}, R^{s} is acetate, adipate, benzoate, benzenesulfonate, citrate, decanoate, chloride, lactate, maleate, methanesulfonate, oxalate, pivalate, propionate, succinate, sulfate, tartrate, or trifluoroacetate; or
a pharmaceutically acceptable salt thereof.

### Clause 8

8. The compound of clause 6 wherein
X8 is

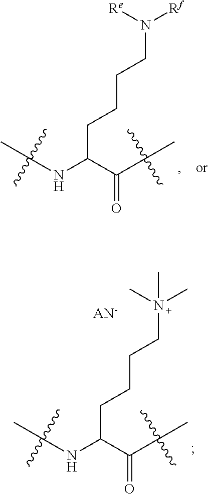

wherein:
   AN⁻as above defined for A, R, X3, X12, R1, R2, R5, R6, R8, R15, R16 and R^{a}, R^{b}, R^{d}, R^{f}, R^{h}, Rⁱ, R^{j}, R^{k}, R^{m}, Rⁿ, R^{o}, R^{s} is acetate, adipate, benzoate, benzenesulfonate, citrate, decanoate, chloride, lactate, maleate, methanesulfonate, oxalate, pivalate, propionate, succinate, sulfate, tartrate, or trifluoroacetate; or
a pharmaceutically acceptable salt thereof.
   Clause 9. 9. The compound of clause 5 selected from
cPEG3aCO-Pen(3)—N(N(Me)2)-T-7MeW—K(NMeAc)-Pen(3)-TMAPF-2Nal-THP-K(NMeAc)—N-3Pya-Sar-CO(NHPEG3a);
MeCO-Pen(3)—N(N(Me)2)-T-7MeW—K(NMeAc)-Pen(3)-TMAPF-2Nal-THP-K(NMeAc)—N-3Pya-Sar-CO(NHPEG3a);
cPEG3aCO-Pen(3)—N(N(Me)2)-T-7MeW—K(NMeAc)-Pen(3)-APEG3F-2Nal-THP-A-N-3Pya-Sar-CON(Me)2;
cPEG3aCO-Pen(3)—N(N(Me)2)-T-7MeW—K(NMeAc)-Pen(3)-TMAPF-2Nal-THP-A-N-3Pya-Sar-CON(Me)2;
MeCO-Pen(3)—N(N(Me)2)-T-7MeW-Dab(NMeAc)-Pen(3)-AEF-2Nal-THP-AIB-N-3Pya-Sar-CON(Me)2;
cPEG3aCO-Pen(3)—N(N(Me)2)-T-7MeW-AIB-Pen(3)-AEF-2Nal-THP-AIB-N-3Pya-Sar-CON(Me)2;
MeCO-Pen(3)—N(N(Me)2)-T-7MeW—K(NMeAc)-Pen(3)-TMAPF-2Nal-THP-APEG2Ser-N-3Pya-Sar-CON(Me)2;
MeCO-Pen(3)—N(N(Me)2)-T-7MeW-APEG2Ser(S*)-Pen(3)-APEG3F-2Nal-THP-APEG2Ser(S*)—N-3Pya-Sar-CON(Me)2;
MeCO-Pen(3)—N(N(Me)2)-T-7MeW-APEG2Ser(R*)-Pen(3)-APEG3F-2Nal-THP-APEG2Ser(S*)—N-3Pya-Sar-CON(Me)2;
MeCO-Pen(3)—N(N(Me)2)-T-7MeW—K(NMeAc)-Pen(3)-APEG3F-2Nal-THP-APEG2Ser(S*)—N-3Pya-Sar-CON(Me)2;
cPEG3aCO-Pen(3)—N(N(Me)2)-T-7MeW—K(NMeAc)-Pen(3)-TMAPF-2Nal-THP-Cit-N-3Pya-Sar-CON(Me)2;
cPEG3aCO-Pen(3)—N(N(Me)2)-T-7MeW—K(NMeAc)-Pen(3)-AEF-2Nal-diFCpx-Dab(NMeAc)—N-3Pya-Sar-CON(Me)2;
cPEG3aCO-Pen(3)—N(N(Me)2)-T-7MeW—K(NMeAc)-Pen(3)-AEF-2Nal-diFCpx-Dab(NMeAc)—N-3Pya-Sar-CON(Me)2;
cPEG3aCO-Pen(3)—N(N(Me)2)-T-7MeW—K(Ac)-Pen(3)-TMAPF-2Nal-THP-Dab(NMeAc)—N-3Pya-Sar-CON(Me)2;
cPEG3aCO-Pen(3)—N(N(Me)2)-T-7MeW—K(NMeAC)-Pen(3)-TMAPF-2Nal-THP-Dab(NMeAc)—N-3Pya-Sar-CON(Me)2;
MeCO-Pen(3)—N(N(Me)2)-T-7MeW-Dab(NMecarn)-Pen(3)-APEG3F-2Nal-THP-Dab(NMeAc)—N-3Pya-Sar-CON(Me)2;
MeCO-k(d)-Pen(3)—N(N(Me)2)-T-7MeW—K(NMeAc)-Pen(3)-APEG3F-2Nal-THP-Dab(NMeAc)—N-3Pya-Sar-CON(Me)2;
cPEG3aCO-Pen(3)—N(N(Me)2)-T-7MeW—K(NMeAc)-Pen(3)-APEG3F-2Nal-THP-Dab(NMeAc)—N-3Pya-Sar-CON(Me)2;
cPEG3aCO-Pen(3)—N(N(Me)2)-T-7MeW—K(NMeAc)-Pen(3)-APEG3F-2Nal-THP-Dab(NMeAc)—N-3Pya-Sar-CON(Me)2;
mPEG3CO-SP6-Pen(3)—N(N(Me)2)-T-7MeW—K(NMeAc)-Pen(3)-APEG3F-2Nal-THP-Dab(NMeAc)—N-3Pya-Sar-CON(Me)2;
cPEG3aCO-Pen(3)—N(N(Me)2)-T-7MeW-Dab(NMecPEG3a)-Pen(3)-AEF-2Nal-THP-Dab(NMeAc)—N-3Pya-Sar-CON(Me)2;
cPEG3aCO-Pen(3)—N(N(Me)2)-T-7MeW—K(NMeAc)-Pen(3)-AEF-2Nal-THP-Dab(NMeAc)—N-3Pya-Sar-CON(Me)2;
cPEG5aCO-Pen(3)—N(N(Me)2)-T-7MeW-Q(N(Me)2)-Pen(3)-AEF-2Nal-THP-Dab(NMeAc)—N-3Pya-Sar-CON(Me)2;
MeCO-Pen(3)—N(N(Me)2)-T-7MeW-Dab(NMeAc)-Pen(3)-AEF-2Nal-THP-Dab(NMeAc)—N-3Pya-Sar-CON(Me)2;
cPEG3aCO-Pen(3)—N(N(Me)2)-T-7MeW-Dab(NMeAc)-Pen(3)-AEF-2Nal-THP-Dab(NMeAc)—N-3Pya-Sar-CON(Me)2;
cPEG3aCO-Pen(3)—N(N(Me)2)-T-7MeW-Q(N(Me)2)-Pen(3)-AEF-2Nal-THP-Dab(NMeAc)—N-3Pya-Sar-CON(Me)2;
cPEG5aCO-Pen(3)—N(N(Me)2)-T-7MeW-Dab(NMeAc)-Pen(3)-AEF-2Nal-THP-Dab(NMeAc)—N-3Pya-Sar-CON(Me)2;
MeCO-Pen(3)—N(N(Me)2)-T-7MeW-Dab(NMecarn)-Pen(3)-APEG3F-2Nal-THP-Dab(NMecarn)-N-3Pya-Sar-CON(Me)2;
MeCO-Pen(3)—N(N(Me)2)-T-7MeW-Dab(NMeCOmPEG6)-Pen(3)-AEF-2Nal-THP-Dab(NMeCOmPEG6)—N-3Pya-Sar-CON(Me)2;
cPEG5aCO-Pen(3)—N(N(Me)2)-T-7MeW-Dab(NMecPEG2a)-Pen(3)-AEF-2Nal-THP-Dab(NMecPEG2a)—N-3Pya-Sar-CON(Me)2;
MeCO-Pen(3)—N(N(Me)2)-T-7MeW-Dab(NMecPEG3a)-Pen(3)-AEF-2Nal-THP-Dab(NMecPEG3a)—N-3Pya-Sar-CON(Me)2;
5cpaCO-Pen(3)—N(N(Me)2)-T-7MeW—K(NMeAc)-Pen(3)-AEF(N(Me)2)-2Nal-THP-Dap(Ac)—N-3Pya-Sar-CON(Me)2;
MeCO-Pen(3)—N(N(Me)2)-T-7MeW—K(NMeAc)-Pen(3)-AEF(N(Me)2)-2Nal-THP-Dap(Ac)—N-3Pya-Sar-CON(Me)2;
cPEG3aCO-Pen(3)—N(N(Me)2)-T-7MeW—K(NMeAc)-Pen(3)-APEG3F-2Nal-THP-Dap(NMeAc)—N-3Pya-Sar-CON(Me)2;
cPEG3aCO-Pen(3)—N(N(Me)2)-T-7MeW—K(Ac)-Pen(3)-TMAPF-2Nal-THP-Dap(NMeAc)—N-3Pya-Sar-CON(Me)2;
cPEG3aCO-Pen(3)—N(N(Me)2)-T-7MeW—K(NMeAc)-Pen(3)-APEG3F-2Nal-THP-E-N-3Pya-Sar-CON(Me)2;
cPEG3aCO-Pen(3)—N(N(Me)2)-T-7MeW-Dab(NMeAc)-Pen(3)-AEF-2Nal-THP-E-N-3Pya-Sar-CONH2;
cPEG3aCO-Pen(3)—N(N(Me)2)-T-7MeW—K(NMeAc)-Pen(3)-TMAPF-2Nal-THP-E-N-3Pya-Sar-CON(Me)2;
MeCO-hk(Me)₃-Pen(3)—N(N(Me)2)-T-7MeW—K(NMeAc)-Pen(3)-AEF-2Nal-THP-E-N-3Pya-Sar-CON(Me)2;
MeCO-Pen(3)—N(N(Me)2)-T-7MeW-Dab(NMeCOmPEG6)-Pen(3)-AEF-2Nal-THP-E-N-3Pya-Sar-CON(Me)2;
MeCO-Pen(3)—N(N(Me)2)-T-7MeW-Dab(NMeCarn)-Pen(3)-AEF-2Nal-THP-E-N-3Pya-Sar-CON(Me)2;
cPEG5aCO-Pen(3)—N(N(Me)2)-T-7MeW-Dab(NMecPEG5a)-Pen(3)-AEF-2Nal-THP-E-N-3Pya-Sar-CON(Me)2;
mPEG6CO-SP6-Pen(3)—N(N(Me)2)-T-7MeW-Dab(NMecPEG5a)-Pen(3)-AEF-2Nal-THP-E-N-3Pya-Sar-CON(Me)2;
MeCO-Pen(3)—N(N(Me)2)-T-7MeW-Dab(NMecarn)-Pen(3)-AEF-2Nal-THP-E-N-3Pya-Sar-CON(Me)2;
MeCO-Pen(3)—N(N(Me)2)-T-7MeW-Dab(NMecPEG2a)-Pen(3)-AEF-2Nal-THP-E-N-3Pya-Sar-CON(Me)2;
MeCO-Pen(3)—N(N(Me)2)-T-7MeW-Dab(NMeAc)-Pen(3)-AEF-2Nal-THP-E-N-3Pya-Sar-CON(Me)2;
MeCO-Pen(3)—N(N(Me)2)-T-7MeW-Q(N(Me)2)-Pen(3)-AEF(N(Me)2)-2Nal-THP-E-N-3Pya-Sar-CON(Me)2;
MeCO-Pen(3)—N(N(Me)2)-T-7MeW—K(NMeAc)-Pen(3)-AEF(N(Me)2)-2Nal-THP-E-N-3Pya-Sar-CON(Me)2;
MeCO-k(Me)₃-Pen(3)—N(N(Me)2)-T-7MeW—K(NMeAc)-Pen(3)-AEF(N(Me)2)-2Nal-THP-E-N-3Pya-Sar-CON(Me)2;
CF3CO-Pen(3)—N(N(Me)2)-T-7MeW—K(NMeAc)-Pen(3)-AEF(N(Me)2)-2Nal-THP-E-N-3Pya-Sar-CON(Me)2;
5cpaCO-Pen(3)—N(N(Me)2)-T-7MeW-Q(N(Me)2)-Pen(3)-AEF(N(Me)2)-2Nal-THP-E-N-3Pya-Sar-CON(Me)2;
CF3CO—K(Me)₃-Pen(3)—N(N(Me)2)-T-7MeW—K(NMeAc)-Pen(3)-AEF(N(Me)2)-2Nal-THP-E-N-3Pya-Sar-CON(Me)2;
cPEG3aCO-Pen(3)—N(N(Me)2)-T-7MeW—K(NMeAc)-Pen(3)-APEG3F-2Nal-THP—F—N-3Pya-Sar-CON(Me)2;
cPEG3aCO-Pen(3)—N(N(Me)2)-T-7MeW—K(NMeAc)-Pen(3)-APEG3F-2Nal-THP—K(Ac)—N-3Pya-Sar-CON(Me)2;
MeCO-Pen(3)—N(N(Me)2)-T-7MeW—K(Me)₃-Pen(3)-AEF(N(Me)2)-2Nal-THP-K(Me)₃—N-3Pya-Sar-CON(Me)2;
cPEG3aCO-Pen(3)—N(N(Me)2)-T-7MeW—K(NMeAc)-Pen(3)-TMAPF-2Nal-Achx-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-Acpx-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-Aib-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-Chg-K(NMeAc)—N-3Pya-Sar-CON(Me)2;
cPEG3aCO-Pen(3)—N(N(Me)2)-T-7MeW—K(NMeAc)-Pen(3)—Y(OTz1TMA4)-2Nal-THP-K(NMeAc)—N-3Pya-Sar-CON(Me)2;
cPEG3aCO-Pen(3)—N(N(Me)2)-T-7(3NAcPh)W—K(NMeAc)-Pen(3)-MMoEF-2Nal-THP-K(NMeAc)—N-3Pya-Sar-CON(Me)2;
cPEG3aCO-Pen(3)—N(N(Me)2)-T-7MeW-Dap(NMeAc)-Pen(3)-TMAPF-2Nal-THP-K(NMeAc)—N-3Pya-Sar-CON(Me)2;
cPEG2gCO-Pen(3)—N(N(Me)2)-T-7MeW—K(NMeAc)-Pen(3)-APEG3F-2Nal-THP-K(NMeAc)—N-3Pya-Sar-CON(Me)2;
cPEG3gCO-Pen(3)—N(N(Me)2)-T-7MeW—K(NMeAc)-Pen(3)-APEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-GPEG3F-2Nal-THP-K(NMeAc)—N-3Pya-Sar-CON(Me)2;
4cpgCO-Pen(3)—N(N(Me)2)-T-7MeW—K(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(PEG2PEG2gEC14)-Pen(3)-TMAPF-2Nal-THP-K(NMeAc)—N-3Pya-Sar-CON(Me)2;
cPEG3aCO-Pen(3)—N(N(Me)2)-T-7MeW—K(4cpg)-Pen(3)-TMAPF-2Nal-THP-K(NMeAc)—N-3Pya-Sar-CON(Me)2;
cPEG3gCO-Pen(3)—N(N(Me)2)-T-7MeW—K(NMeAc)-Pen(3)-TMAPF-2Nal-THP-K(NMeAc)—N-3Pya-Sar-CON(Me)2;
cPEG2gCO-Pen(3)—N(N(Me)2)-T-7MeW—K(NMeAc)-Pen(3)-TMAPF-2Nal-THP-K(NMeAc)—N-3Pya-Sar-CON(Me)2;
C14gEPEG2PEG2CO-k(PEG2PEG2gEC14)-Pen(3)—N(N(Me)2)-T-7MeW-Dab(NMeAc)-Pen(3)-TMAPF-2Nal-THP-K(NMeAc)—N-3Pya-Sar-CON(Me)2;
C12gEPEG2PEG2CO-k(PEG2PEG2gEC12)-Pen(3)—N(N(Me)2)-T-7MeW-Dab(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(cPEG3a)-Pen(3)-TMAPF-2Nal-THP-K(NMeAc)—N-3Pya-Sar-CON(Me)2;
cPEG3aCO-k(PEG2PEG2gEC12)-Pen(3)—N(N(Me)2)-T-7MeW—K(PEG2PEG2gEC12)-Pen(3)-TMAPF-2Nal-THP-K(NMeAc)—N-3Pya-Sar-CON(Me)2;
cPEG3aCO-k(PEG2PEG2gEC14)-Pen(3)—N(N(Me)2)-T-7MeW—K(NMeAc)-Pen(3)-TMAPF-2Nal-THP-K(NMeAc)—N-3Pya-Sar-CON(Me)2;
cPEG3aCO-k(PEG2PEG2gEC12)-Pen(3)—N(N(Me)2)-T-7MeW—K(NMeAc)-Pen(3)-TMAPF-2Nal-THP-K(NMeAc)—N-3Pya-Sar-CON(Me)2;
C14gEPEG2PEG2CO-APEG2Ser(S*)-Pen(3)—N(N(Me)2)-T-7MeW—K(PEG2PEG2gEC14)-Pen(3)-APEG3F-2Nal-THP-K(NMeAc)—N-3Pya-Sar-CON(Me)2;
C12gEPEG2PEG2CO-APEG2Ser(S*)-Pen(3)—N(N(Me)2)-T-7MeW—K(PEG2PEG2gEC12)-Pen(3)-APEG3F-2Nal-THP-K(NMeAc)—N-3Pya-Sar-CON(Me)2;
C14gEPEG2PEG2CO-APEG2Ser(S*)-Pen(3)—N(N(Me)2)-T-7MeW—K(NMeAc)-Pen(3)-APEG3F-2Nal-THP-K(NMeAc)—N-3Pya-Sar-CON(Me)2;
C12gEPEG2PEG2CO-APEG2Ser(S*)-Pen(3)—N(N(Me)2)-T-7MeW—K(NMeAc)-Pen(3)-APEG3F-2Nal-THP-K(NMeAc)—N-3Pya-Sar-CON(Me)2;
cPEG3aCO-Pen(3)—N(N(Me)2)-T-7MeW-Dab(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW-Cit-Pen(3)-TMAPF-2Nal-THP-K(NMeAc)—N-3Pya-Sar-CON(Me)2;
MeCO-r-Pen(3)—N(N(Me)2)-T-7MeW—K(NMeAc)-Pen(3)-TMAPF-2Nal-THP-K(NMeAc)—N-3Pya-Sar-CON(Me)2;
MeCO-hk(Me)₃-Pen(3)—N(N(Me)2)-T-7MeW—K(NMeAc)-Pen(3)-TMAPF-2Nal-THP-K(NMeAc)—N-3Pya-Sar-CON(Me)2;
5cpaCO-Pen(3)—N(N(Me)2)-T-7MeW—K(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(NMeAc)-Pen(3)-hFTMAPF-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4Tz1DMA4mPEG)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4Tz1MMo4)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4Tz1TMA5)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4Tz1TMA3)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4Tz1TMA2)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4Tz1TMA1)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4N3)-2Nal-THP-K(NMeAc)—N-3Pya-Sar-CON(Me)2;
cPEG3aCO-Pen(3)—N(N(Me)2)-T-7MeW—K(Me)₃-Pen(3)-TMAPF-2Nal-THP-K(NMeAc)—N-3Pya-Sar-CON(Me)2;
C14gEPEG2PEG2CO-hk(Me)₃-Pen(3)—N(N(Me)2)-T-7MeW—K(PEG2PEG2gEC14)-Pen(3)-TMAPF-2Nal-THP-K(NMeAc)—N-3Pya-Sar-CON(Me)2;
C14gEPEG2PEG2CO-hk(Me)₃-Pen(3)—N(N(Me)2)-T-7MeW—K(NMeAc)-Pen(3)-TMAPF-2Nal-THP-K(NMeAc)—N-3Pya-Sar-CON(Me)2;
C12gEPEG2PEG2CO-hk(Me)₃-Pen(3)—N(N(Me)2)-T-7MeW—K(PEG2PEG2gEC12)-Pen(3)-TMAPF-2Nal-THP-K(NMeAc)—N-3Pya-Sar-CON(Me)2;
C12gEPEG2PEG2CO-hk(Me)₃-Pen(3)—N(N(Me)2)-T-7MeW—K(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(N(Me)2)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF-2Nal-THP-K(NMeAc)—N-3Pya-Sar-CON(Me)2;
MeCO-APEG2Ser(S*)-Pen(3)—N(N(Me)2)-T-7MeW—K(NMeAc)-Pen(3)-APEG3F-2Nal-THP-K(NMeAc)—N-3Pya-Sar-CON(Me)2;
MeCO-APEG2Ser(R*)-Pen(3)—N(N(Me)2)-T-7MeW—K(NMeAc)-Pen(3)-APEG3F-2Nal-THP-K(NMeAc)—N-3Pya-Sar-CON(Me)2;
cPEG3aCO-Pen(3)—N(N(Me)2)-T-7MeW-Q(N(Me)2)-Pen(3)-TMAPF-2Nal-THP-K(NMeAC)—N-3Pya-Sar-CON(Me)2;
cPEG3aCO-Pen(3)—N(N(Me)2)-T-7MeW—K(NMeAc)-Pen(3)—Y(OZOXIMECh)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OEOXIMECh)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4G)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4Tz1MME)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4Tz1TMA4)-2Nal-THP-K(NMeAc)—N-3Pya-Sar-CON(Me)2;
cPEG3aCO-Pen(3)—N(N(Me)2)-T-7MeW—K(NMeAc)-Pen(3)-dFPPEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-NPyPEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-TMA8F-2Nal-THP-K(NMeAc)—N-3Pya-Sar-CON(Me)2;
cPEG3aCO-Pen(3)—N(N(Me)2)-T-7(3NAcPh)W—K(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(NMeAc)-Pen(3)-TMA6F-2Nal-THP-K(NMeAc)—N-3Pya-Sar-CON(Me)2;
cPEG3aCO-Pen(3)—N(N(Me)2)-T-7MeW—K(NMeAc)-Pen(3)-Diazabiclyclooctane6F-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OTzlChC16)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OTzlC1 aC8)-2Nal-THP-K(NMeAc)—N-3Pya-Sar-CON(Me)2;
cPEG3aCO-Pen(3)—N(N(Me)2)-T-7MeW—K(NMeAc)-Pen(3)-TzlChmPEG3-2Nal-THP-K(NMeAc)—N-3Pya-Sar-CON(Me)2;
cPEG3aCO-Pen(3)—N(N(Me)2)-T-7MeW—K(NMeAc)-Pen(3)-TzlChmPEG-2Nal-THIP-K(NMeAc)—N-3Pya-Sar-CON(Me)2;
cPEG3aCO-Pen(3)—N(N(Me)2)-T-7MeW—K(NMeAc)-Pen(3)-Tzl(Ch)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OTzlCh)-2Nal-THIP-K(NMeAc)—N-3Pya-Sar-CON(Me)2;
cPEG3aCO-Pen(3)—N(N(Me)2)-T-7MeW—K(NMeAc)-Pen(3)—Y(OTzlChmPEG)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OTz1TMA4)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OTzlChmPEG3)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(SPD)-2Nal-THP-K(NMeAc)—N-3Pya-Sar-CON(Me)2;
cPEG3aCO-Pen(3)—N(N(Me)2)-T-7MeW—K(NMeAc)-Pen(3)-MMoPF-2Nal-THIP-K(NMeAc)—N-3Pya-Sar-CON(Me)2;
cPEG3aCO-Pen(3)—N(N(Me)2)-T-7MeW—K(NMeAc)-Pen(3)-PiperazinequatF-2Nal-THP-K(NMeAc)—N-3Pya-Sar-CON(Me)2;
cPEG3aCO-Pen(3)—N(N(Me)2)-T-7MeW—K(NMeAc)-Pen(3)-mPEG3 TMA4F-2Nal-THP-K(NMeAc)—N-3Pya-Sar-CON(Me)2;
cPEG3aCO-Pen(3)—N(N(Me)2)-T-7MeW—K(NMeAc)-Pen(3)-morfTMA4F-2Nal-THP-K(NMeAc)—N-3Pya-Sar-CON(Me)2;
cPEG3aCO-Pen(3)—N(N(Me)2)-T-7MeW—K(NMeAc)-Pen(3)-DMTASF-2Nal-THP-K(NMeAc)—N-3Pya-Sar-CON(Me)2;
MeCO-Pen(3)—N(N(Me)2)-T-7MeW-APEG2Ser(R*)-Pen(3)-APEG3F-2Nal-THP-K(NMeAc)—N-3Pya-Sar-CON(Me)2;
MeCO-Pen(3)—N(N(Me)2)-T-7MeW-APEG2Ser(S*)-Pen(3)-APEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(BisPEG2a)(S*)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(BisPEG2a)(RS)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(aPEG2a)-2Nal-THP-K(NMeAc)—N-3Pya-Sar-CON(Me)2;
cPEG3aCO-Pen(3)—N(N(Me)2)-T-7MeW—K(NMeAc)-Pen(3)-TBAPEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-MTASF-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(NMe3)-2Nal-THP-K(NMeAc)—N-3Pya-Sar-CON(Me)2;
cPEG3aCO-Pen(3)—N(N(Me)2)-T-7MeW—K(NMeAc)-Pen(3)—YC8CO(NHPEG3a)-2Nal-THP-K(NMeAc)—N-3Pya-Sar-CON(Me)2;
cPEG3aCO-Pen(3)—N(N(Me)2)-T-7MeW-APEG2Ser(S*)-Pen(3)-TMAPF-2Nal-THP-K(NMeAc)—N-3Pya-Sar-CON(Me)2;
cPEG3aCO-Pen(3)—N(N(Me)2)-T-7MeW-APEG2Ser(R*)-Pen(3)-TMAPF-2Nal-THP-K(NMeAc)—N-3Pya-Sar-CON(Me)2;
cPEG3aCO-Pen(3)—N(N(Me)2)-T-7MeW-APEG2Ser-Pen(3)-AEF-2Nal-THP-K(NMeAc)—N-3Pya-Sar-CON(Me)2;
cPEG3aCO-Pen(3)—N(N(Me)2)-T-7MeW-APEG2Ser(R*)-Pen(3)-AEF-2Nal-THP-K(NMeAc)—N-3Pya-Sar-CON(Me)2;
cPEG3aCO-Pen(3)—N(N(Me)2)-T-7MeW—K(Ac)-Pen(3)-APEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-YC8COPip-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(NMeBismPEG3)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(NMe2mPEG3)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(PEG2a)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(NMePEG2a)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(C9OH)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(MePrpa)-2Nal-THP-K(NMeAc)—N-3Pya-Sar-CON(Me)2;
MeCO-APEG2ser-Pen(3)—N(N(Me)2)-T-7MeW—K(NMeAc)-Pen(3)-APEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-NPyEF-2Nal-THP-K(NMeAc)—N-3Pya-Sar-CON(Me)2;
cPEG3aCO-Pen(3)—N(N(Me)2)-T-7MeW—K(NMeAc)-Pen(3)-TMA3F-2Nal-THP-K(NMeAc)—N-3Pya-Sar-CON(Me)2;
cPEG3aCO-Pen(3)—N(N(Me)2)-T-7MeW—K(NMeAc)-Pen(3)-TMA3F-2Nal-THP-K(NMeAc)—N-3Pya-Sar-CON(Me)2;
cPEG3aCO-Pen(3)—N(N(Me)2)-T-7MeW—K(NMeAc)-Pen(3)-mPEG2TMA2F-2Nal-THP-K(NMeAc)—N-3Pya-Sar-CON(Me)2;
cPEG3aCO-Pen(3)—N(N(Me)2)-T-7MeW—K(NMeAc)-Pen(3)-mPEG2TMA4F-2Nal-THP-K(NMeAc)—N-3Pya-Sar-CON(Me)2;
cPEG3aCO-Pen(3)—N(N(Me)2)-T-7MeW—K(NMeAc)-Pen(3)-TMA4F-2Nal-THP-K(NMeAc)—N-3Pya-Sar-CON(Me)2;
mPEG3CO-SP6-Pen(3)—N(N(Me)2)-T-7MeW—K(NMeAc)-Pen(3)-APEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-MPzPEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-MMPEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-ACHMF-2Nal-THP-K(NMeAc)—N-3Pya-Sar-CON(Me)2;
MeCO-Pen(3)—N(N(Me)2)-T-7MeW—K(NMePEG3a)-Pen(3)-APEG3F-2Nal-THP-K(NMeAc)—N-3Pya-Sar-CON(Me)2;
MeCO-k(Me)₃-Pen(3)—N(N(Me)2)-T-7MeW—K(NMeAc)-Pen(3)-APEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-DMMMF-2Nal-THP-K(NMeAc)—N-3Pya-Sar-CON(Me)2;
MeCO-Pen(3)—N(N(Me)2)-T-7MeW-Dab(NMecarn)-Pen(3)-APEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-ACHMF-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF((Ch)cPEG3a)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(AcCh)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(NsCh)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(BisMEP))-2Nal-THP-K(NMeAc)—N-3Pya-Sar-CON(Me)2;
cPEG3aCO-Pen(3)—N(N(Me)2)-T-7MeW—K(NMeAc)-Pen(3)-DMPMF-2Nal-THP-K(NMeAc)—N-3Pya-Sar-CON(Me)2;
cPEG3aCO-Pen(3)—N(N(Me)2)-T-7MeW—K(NMeAc)-Pen(3)-APEG2F-2Nal-THP-K(NMeAc)—N-3Pya-Sar-CON(Me)2;
cPEG3aCO-Pen(3)—N(N(Me)2)-T-7MeW—K(NMeAc)-Pen(3)-MMoEF-2Nal-THP-K(NMeAc)—N-3Pya-Sar-CON(Me)2;
cPEG3aCO-Pen(3)—N(N(Me)2)-T-7MeW—K(NMeAc)-Pen(3)-4DMPzEF-2Nal-THP-K(NMeAc)—N-3Pya-Sar-CON(Me)2;
MeCO-Pen(3)—N(N(Me)2)-T-7MeW—K(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(NMeAc)-Pen(3)-4DMPEF-2Nal-THP-K(NMeAc)—N-3Pya-Sar-CON(Me)2;
MeCO-Pen(3)—N(N(Me)2)-T-7MeW—K(NMeAc)-Pen(3)-APEG3F-2Nal-TTHP-K(NMeAc)—N-3Pya-Sar-CON(Me)2;
cPEG3aCO-Pen(3)—N(N(Me)2)-T-7MeW—K(NMeAc)-Pen(3)-APEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-THP-K(NMeAc)—N-3Pya-Sar-CON(Me)2;
MeCO-Pen(3)—N(N(Me)2)-T-7MeW—K(NMeAc)-Pen(3)-AEF(N(Me)2)-2Nal-THP-K(NMeAc)—N-3Pya-Sar-CON(Me)2;
CF3CO-Pen(3)—N(N(Me)2)-T-7CF3W—K(NMeAc)-Pen(3)-AEF(N(Me)2)-2Nal-THP-K(NMeAc)—N-3Pya-Sar-CON(Me)2;
CF3CO-Pen(3)—N(N(Me)2)-T-7CF3W—K(NMeAc)-Pen(3)-AEF-2Nal-THP-K(NMeAc)—N-3Pya-Sar-CON(Me)2;
CF3CO-Pen(3)—N(N(Me)2)-T-7MeW—K(NMeAc)-Pen(3)-AEF-2Nal-THP-K(NMeAc)—N-3Pya-Sar-CON(Me)2;
CF3CO-Pen(3)—N(N(Me)2)-T-7MeW—K(NMeAc)-Pen(3)-AEF(N(Me)2)-2Nal-THP-K(NMeAc)—N-3Pya-Sar-CON(Me)2;
MeCO-Pen(3)—N(N(Me)2)-T-7MeW—K(NMeAc)-Pen(3)-APEG3F-2Nal-TTHP-K(NMePEG3a)—N-3Pya-Sar-CON(Me)2;
cPEG3aCO-Pen(3)—N(N(Me)2)-T-7MeW—K(NMeAc)-Pen(3)-TMAPF-2Nal-THP-K(PEG2PEG2gEC12)—N-3Pya-Sar-CON(Me)2;
cPEG3aCO-Pen(3)—N(N(Me)2)-T-7MeW—K(PEG2PEG2gEC12)-Pen(3)-TMAPF-2Nal-THP-K(PEG2PEG2gEC12)—N-3Pya-Sar-CON(Me)2;
cPEG3aCO-k(PEG2PEG2gEC12)-Pen(3)—N(N(Me)2)-T-7MeW—K(NMeAc)-Pen(3)-TMAPF-2Nal-THP-K(PEG2PEG2gEC12)—N-3Pya-Sar-CON(Me)2;
C12gEPEG2PEG2CO-hk(Me)₃-Pen(3)—N(N(Me)2)-T-7MeW—K(NMeAc)-Pen(3)-TMAPF-2Nal-THP-K(PEG2PEG2gEC12)—N-3Pya-Sar-CON(Me)2;
C12gEPEG2PEG2CO-hk(Me)₃-Pen(3)—N(N(Me)2)-T-7MeW—K(NMeAc)-Pen(3)-TMAPF-2Nal-K(PEG2PEG2gEC12)—N-3Pya-Sar-CON(Me)2;
cPEG3aCO-Pen(3)—N(N(Me)2)-T-7MeW—K(PEG2PEG2gEC14)-Pen(3)-TMAPF-2Nal-THP-K(PEG2PEG2gEC14)—N-3Pya-Sar-CON(Me)2;
cPEG3aCO-Pen(3)—N(N(Me)2)-T-7MeW—K(NMeAc)-Pen(3)-TMAPF-2Nal-THP-K(PEG2PEG2gEC14)—N-3Pya-Sar-CON(Me)2;
cPEG3aCO-k(PEG2PEG2gEC14)-Pen(3)—N(N(Me)2)-T-7MeW—K(NMeAc)-Pen(3)-TMAPF-2Nal-THP-K(PEG2PEG2gEC14)—N-3Pya-Sar-CON(Me)2;
C14gEPEG2PEG2CO-hk(Me)₃-Pen(3)—N(N(Me)2)-T-7MeW—K(NMeAc)-Pen(3)-TMAPF-2Nal-THP-K(PEG2PEG2gEC14)—N-3Pya-Sar-CON(Me)2;
cPEG3aCO-Pen(3)—N(N(Me)2)-T-7MeW—K(NMeAc)-Pen(3)-APEG3F-2Nal-THP-L-N-3Pya-Sar-CON(Me)2;
5cpaCO-Pen(3)—N(N(Me)2)-T-7MeW-Q(N(Me)2)-Pen(3)-AEF(N(Me)2)-2Nal-Pip(NMe2)-Q(N(Me)2)—N-3Pya-Sar-CON(Me)2;
cPEG3aCO-Pen(3)—N(N(Me)2)-T-7MeW—K(NMeAC)-Pen(3)-TMAPF-2Nal-THP-Q(N(Me)2)—N-3Pya-Sar-CON(Me)2;
cPEG3aCO-Pen(3)—N(N(Me)2)-T-7MeW-Q(N(Me)2)-Pen(3)-AEF(N(Me)2)-2Nal-THP-Q(N(Me)2)—N-3Pya-Sar-CON(Me)2;
cPEG3aCO-Pen(3)—N(N(Me)2)-T-7MeW-Q(N(Me)2)-Pen(3)-AEF-2Nal-THP-Q(N(Me)2)—N-3Pya-Sar-CON(Me)2;
MeCO-Pen(3)—N(N(Me)2)-T-7MeW-Q(N(Me)2)-Pen(3)-AEF(N(Me)2)-2Nal-THP-Q(N(Me)2)—N-3Pya-Sar-CON(Me)2;
5cpaCO-Pen(3)—N(N(Me)2)-T-7MeW-Q(N(Me)2)-Pen(3)-TMAPF-2Nal-THP-Q(N(Me)2)—N-3Pya-Sar-CON(Me)2;
CF3CO-k(5cpa)-Pen(3)—N(N(Me)2)-T-7MeW-Q(N(Me)2)-Pen(3)-AEF(N(Me)2)-2Nal-THP-Q(N(Me)2)—N-3Pya-Sar-CON(Me)2;
5cpaCO-Pen(3)—N(N(Me)2)-T-7MeW-Q(N(Me)2)-Pen(3)-AEF(N(Me)2)-2Nal-THP-Q(N(Me)2)—N-3Pya-Sar-CON(Me)2;
cPEG3aCO-Pen(3)—N(N(Me)2)-T-7MeW—K(NMeAc)-Pen(3)-APEG3F-2Nal-THP—W—N-3Pya-Sar-CON(Me)2;
cPEG3aCO-Pen(3)—N(N(Me)2)-T-7MeW—K(NMeAc)-Pen(3)-AEF(BisMEPa)-2Nal-THP-K(NMeAc)—N-3Pya-Sar-CON(Me)2;
cPEG3aCO-Pen(3)—N(N(Me)2)-T-7MeW-Dab(NMeAc)-Pen(3)-AEF-2Nal-THP-Dab(NMeAc)—N-3Pya-Sar-CONH(PEG3a);
cPEG3aCO-Pen(3)—N(N(Me)2)-T-7MeW—K(NMeAC)-Pen(3)-TMAPF-2Nal-THP-K(NMeAC)—N-3Pya-NMeK(PEG2PEG2C12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG2C14)—CONH2;
C12gEPEG2PEG2CO-Pen(3)—N(N(Me)2)-T-7MeW—K(Ac)-Pen(3)-TMAPF-2Nal-THP-K(NMeAc)—N-3Pya-NMeK(PEG2PEG2gEC12)—CONH2;
cPEG3aCO-Pen(3)—N(N(Me)2)-T-7MeW—K(PEG2PEG2gEC12)-Pen(3)-TMAPF-2Nal-THP-K(NMeAc)—N-3Pya-NMeK(PEG2PEG2gEC12)—CONH2;
cPEG3aCO-k(PEG2PEG2gEC12)-Pen(3)—N(N(Me)2)-T-7MeW—K(Ac)-Pen(3)-TMAPF-2Nal-THP-K(NMeAc)—N-3Pya-NMeK(PEG2PEG2gEC12)—CONH2;
cPEG3aCO-Pen(3)—N(N(Me)2)-T-7MeW—K(NMeAC)-Pen(3)-TMAPF-2Nal-THP-K(NMeAC)—N-3Pya-NMeK(PEG2PEG2gEC12)—CONH2;
cPEG3aCO-Pen(3)—N(N(Me)2)-T-7MeW—K(Ac)-Pen(3)-TMAPF-2Nal-THP-K(NMeAC)—N-3Pya-NMeK(PEG2PEG2gEC12)—CONH2;
cPEG3aCO-Pen(3)—N(N(Me)2)-T-7MeW—K(Ac)-Pen(3)-TMAPF-2Nal-THP-K(PEG2PEG2gEC12)—N-3Pya-NMeK(PEG2PEG2gEC12)—CONH2;
cPEG3aCO-Pen(3)—N(N(Me)2)-T-7MeW—K(PEG2PEG2gEC14)-Pen(3)-TMAPF-2Nal-THP-K(NMeAc)—N-3Pya-NMeK(PEG2PEG2gEC14)—CONH2;
C14gEPEG2PEG2CO-Pen(3)—N(N(Me)2)-T-7MeW—K(Ac)-Pen(3)-TMAPF-2Nal-THP-K(NMeAc)—N-3Pya-NMeK(PEG2PEG2gEC14)—CONH2;
cPEG3aCO-k(PEG2PEG2gEC14)-Pen(3)—N(N(Me)2)-T-7MeW—K(Ac)-Pen(3)-TMAPF-2Nal-THP-K(NMeAc)—N-3Pya-NMeK(PEG2PEG2gEC14)—CONH2;
cPEG3aCO-Pen(3)—N(N(Me)2)-T-7MeW—K(NMeAC)-Pen(3)-TMAPF-2Nal-THP-K(NMeAC)—N-3Pya-NMeK(PEG2PEG2gEC14)—CONH2;
cPEG3aCO-Pen(3)—N(N(Me)2)-T-7MeW—K(Ac)-Pen(3)-TMAPF-2Nal-THP-K(PEG2PEG2gEC14)—N-3Pya-NMeK(PEG2PEG2gEC14)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG2K(PEG2PEG2gEC12)₂)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG2K(PEG2PEG2PEG2PEG2gEC12)₂)—CONH2;
cPEG3aCO-Pen(3)—N(N(Me)2)-T-7MeW—K(Ac)-Pen(3)-TMAPF-2Nal-THP-K(PEG2PEG2PEG2gEC12))—N-3Pya-NMeK(PEG2PEG2PEG2gEC12)—CONH2;
cPEG3aCO-k(PEG2PEG2PEG2PEG2gEC12)-Pen(3)—N(N(Me)2)-T-7MeW—K(Ac)-Pen(3)-TMAPF-2Nal-THP-K(NMeAc)—N-3Pya-NMeK(PEG2PEG2PEG2PEG2gEC12)—CONH2;
cPEG3aCO-k(PEG2PEG6gEC12)-Pen(3)—N(N(Me)2)-T-7MeW—K(Ac)-Pen(3)-TMAPF-2Nal-THP-K(NMeAc)—N-3Pya-NMeK(PEG2PEG6gEC12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG6gEC12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG6gEC14)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(SP6PEG2PEG2C12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(SP6PEG2PEG2gEC12)—CONH2;
MeCO-Pen(3)—N(N(Me)2)-T-7MeW—K(Ac)-Pen(3)-AEF-2Nal-THP—K(Ac)—N-3Pya-Sar-CONH2;
cPEG3aCO-Pen(3)—N(N(Me)2)-T-7MeW—K(NMeAc)-Pen(3)-AEF-2Nal-THP—K(Ac)—N-3Pya-Sar-CONH2;
mPEG3CO-SP6-Pen(3)—N(N(Me)2)-T-7MeW—K(NMeAc)-Pen(3)-AEF-2Nal-THP—K(Ac)—N-3Pya-Sar-CONH2;
CF3CO-Pen(3)—N(N(Me)2)-T-7MeW—K(Ac)-Pen(3)-AEF-2Nal-THP—K(Ac)—N-3Pya-Sar-CONH2;
cPEG3aCO-Pen(3)—N(N(Me)2)-T-7MeW—K(NMeAc)-Pen(3)-APEG3F-2Nal-THP-K(NMeAc)—N-3Pya-Sar-CONH2;
cPEG3aCO-Pen(3)—N(N(Me)2)-T-7MeW-Dab(NMeAc)-Pen(3)-TMAPF-2Nal-THP-K(NMeAc)—N-3Pya-Sar-CONH2;
cPEG3aCO-Pen(3)—N(N(Me)2)-T-7MeW—K(NMeAc)-Pen(3)-TMAPF-2Nal-THP-K(NMeAc)—N-3Pya-Sar-CONH2;
MeCO-Pen(3)—N(N(Me)2)-T-7MeW—K(NMeAc)-Pen(3)-AEF-2Nal-THP-K(NMeAc)—N-3Pya-Sar-CONH2;
cPEG3aCO-Pen(3)—N(N(Me)2)-T-7MeW-Dab(NMeAc)-Pen(3)-AEF-2Nal-THP-K(NMeAc)—N-3Pya-Sar-CONH2;
cPEG3aCO-Pen(3)—N(N(Me)2)-T-7MeW—K(NMeAc)-Pen(3)-AEF-2Nal-THP-K(NMeAc)—N-3Pya-Sar-CONH2;
cPEG3aCO-Pen(3)—N(N(Me)2)-T-7MeW—K(NMeAc)-Pen(3)-AEF-2Nal-THP-Q(N(Me)2)—N-3Pya-Sar-CONH2;
cPEG3aCO-Pen(3)—N(N(Me)2)-T-7MeW-Q(N(Me)2)-Pen(3)-AEF-2Nal-THP-Q(N(Me)2)—N-3Pya-Sar-CONH2;
mPEG3CO-SP6-Pen(3)—N(N(Me)2)-T-7MeW-Q(N(Me)2)-Pen(3)-AEF-2Nal-THP-Q(N(Me)2)—N-3Pya-Sar-CONH2;
CF3CO-Pen(3)—N(N(Me)2)-T-7MeW—K(NMeAc)-Pen(3)-AEF(N(Me)2)-2Nal-THP-Q(N(Me)2)—N-3Pya-Sar-CONH2;
mPEG3CO-SP6-Pen(3)—N(N(Me)2)-T-7MeW—K(NMeAc)-Pen(3)-AEF-2Nal-THP-QNMe2-N-3Pya-Sar-CONH2;
cPEG3aCO-Pen(3)—N(NMe2)-T-7MeW—K(NMeAc)-Pen(3)-AEF(NHCh)-2Nal-THP-K(NMeAc)—N-3Pya-Sar-CON(Me)2;
   or a pharmaceutically acceptable salt thereof.

### Clause 10

10. The compound of clause 3 wherein:

R⁹ is —NHCH₃, —N(CH₃)₂, —NH(CH₂CH₂O)₂CH₃, —N(CH₃)(CH₂CH₂O)₂CH₃, —NH(CH₂CH₂O)_{y}R^{s},

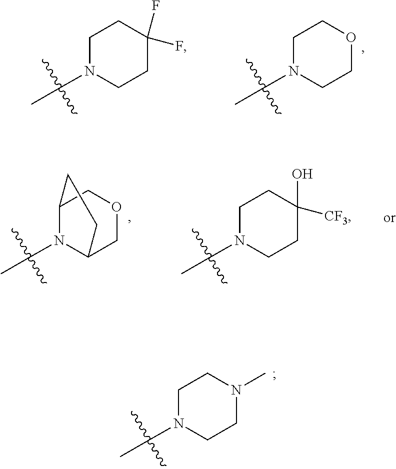

wherein:
   y is 2, 4, or 6;
   R^{s} is —CH₃, or

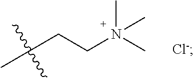

or
a pharmaceutically acceptable salt thereof.

### Clause 11

11. A compound of Formula (IV):

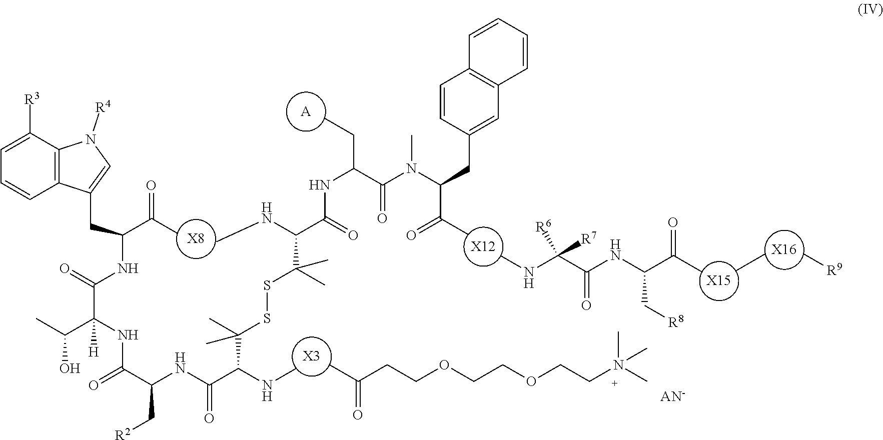

wherein:
A is

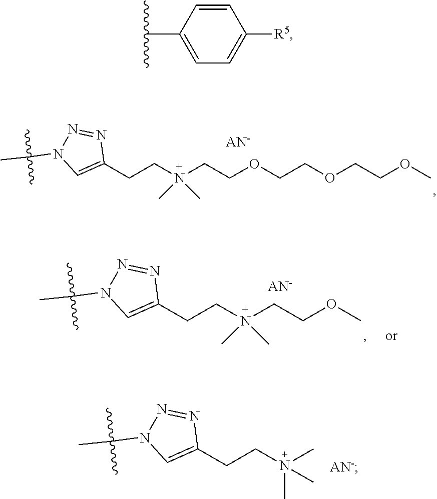

wherein
   R⁵ is —OCHF₂, —O(CH₂)₉CO₂H,

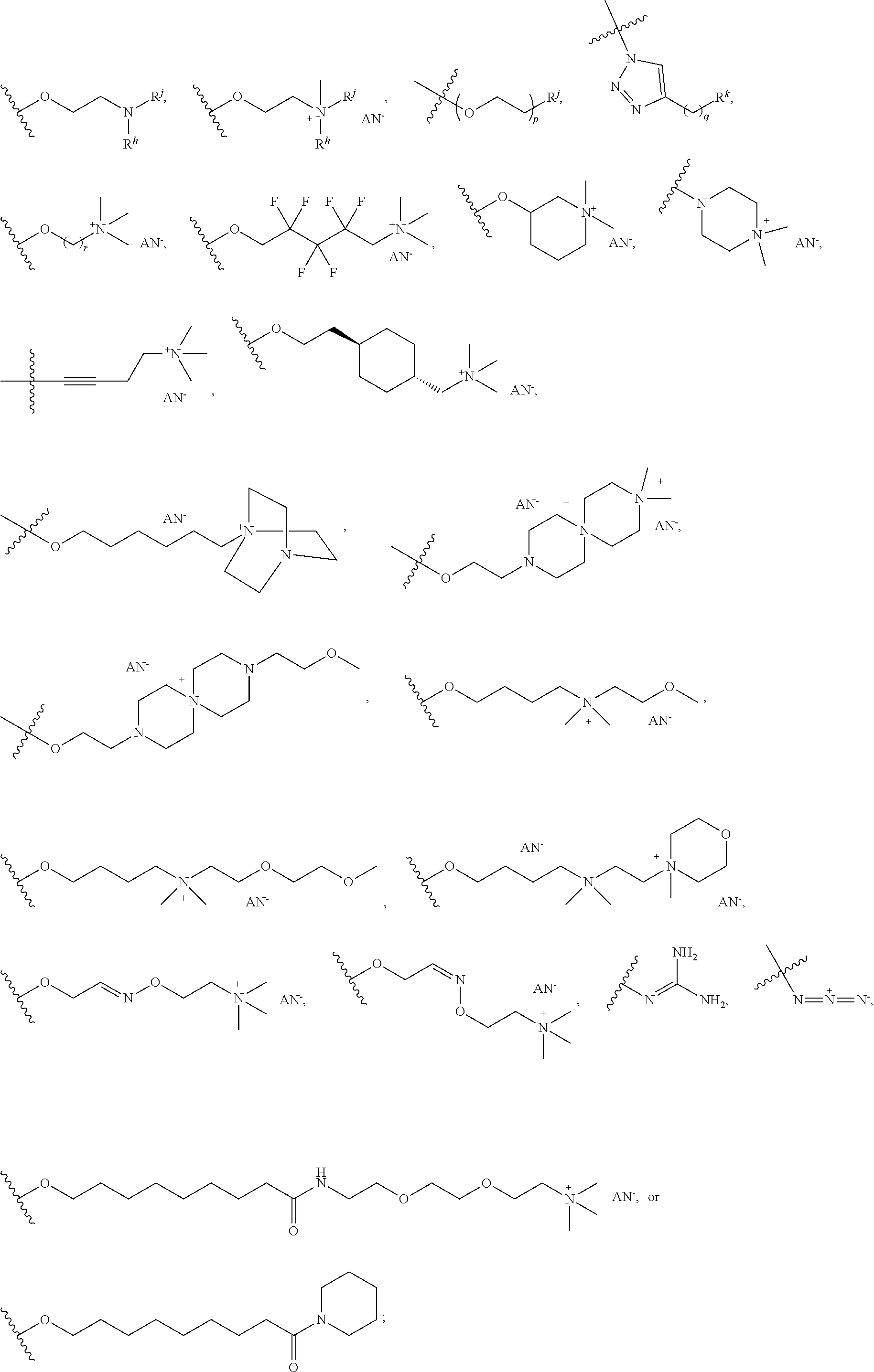

wherein:
   R^{h} is H, —CH₃, —C(O)CH₃, —C(NH)NH₂, —(CH₂)₃O(CH₂)₂OCH₃, —CH₂CH₂OCH₃, —(CH₂CH₂O)₃CH₃, —(CH₂CH₂O)₆CH₃,

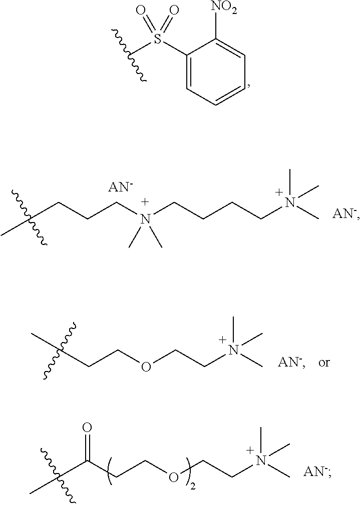

Rⁱ is H, —CH₃, —(CH₂)₃O(CH₂)₂OCH₃,

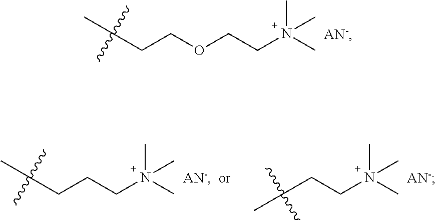

p is 1, 2 or 3;
R^{j} is

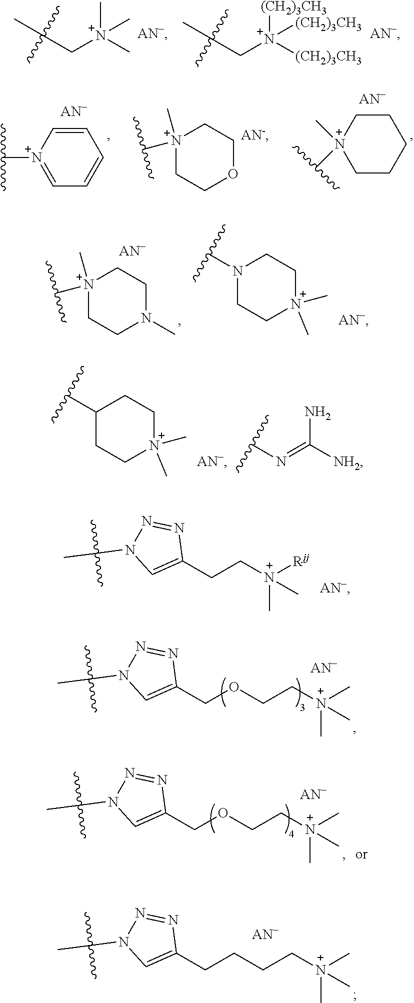

q is 1, 2, 3, 4, or 5;
R^{k} is —OCH₃,

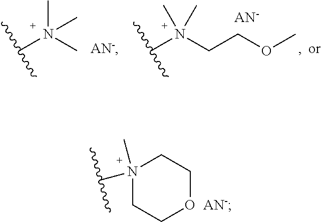

r is 3, 4, 5, 6, or 8;
wherein:
   R^{jj} is H, —(CH₂)₇CH₃, —(CH₂)₁₅CH₃, —(CH₂)₂OCH₃, or —(CH₂CH₂O)₃CH₃;
X3 is a direct bond, (D) arginine, (D) lysine substituted with R^{b},

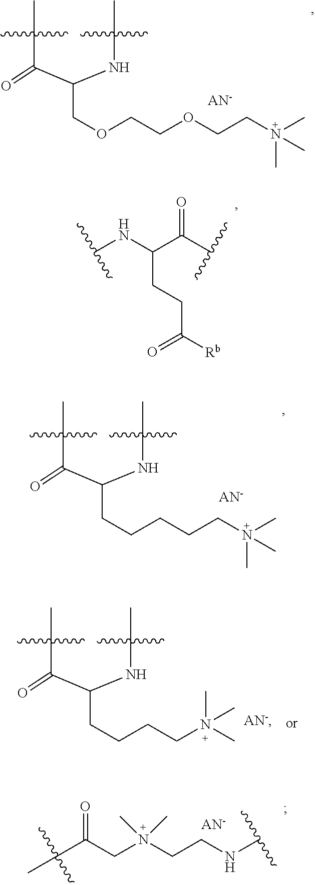

wherein:
   R^{b} is

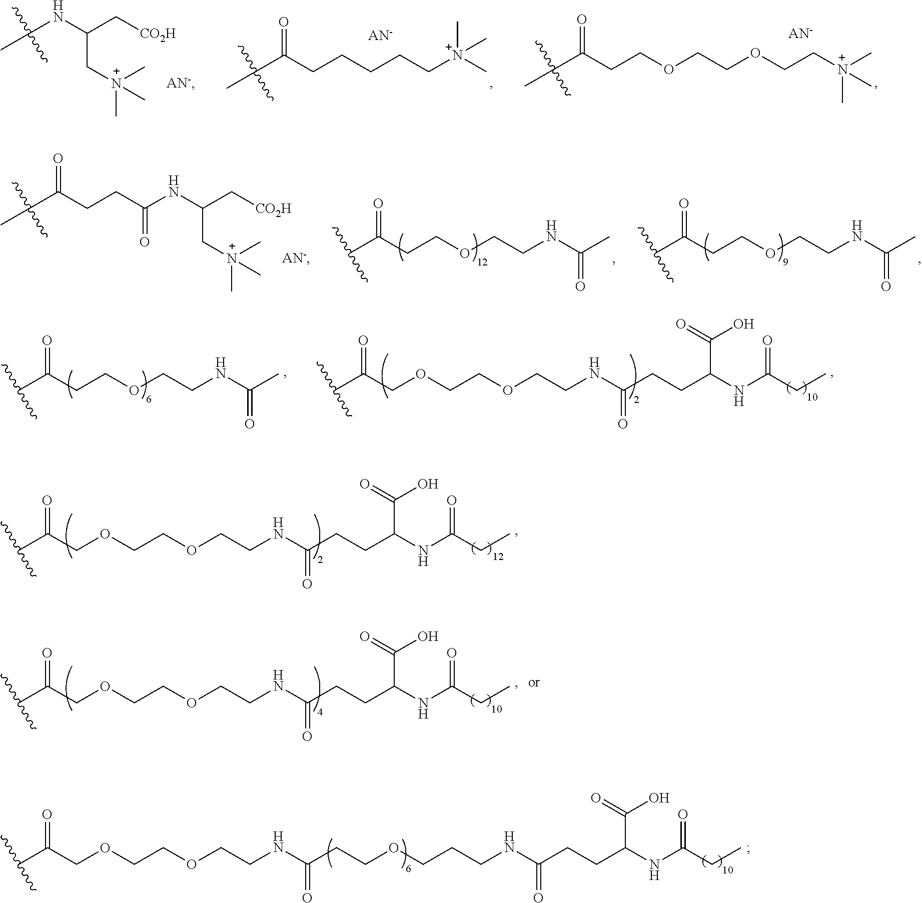

R² is H, —CH(CH₃)₂,

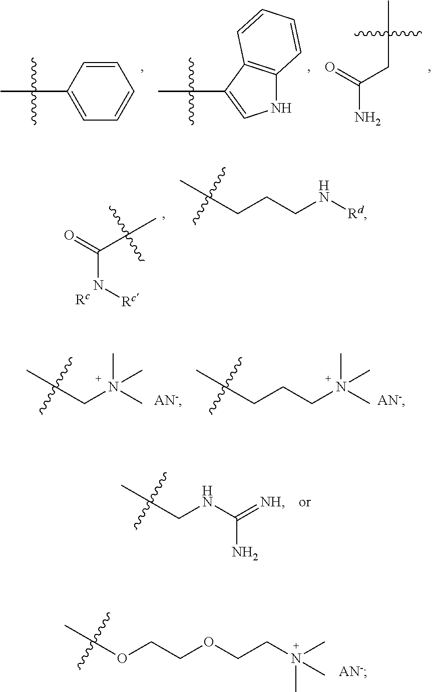

wherein:
   R^{c} is H, or —CH₃;
   R^{c}′ is H, or —CH₃;
   R^{d} is

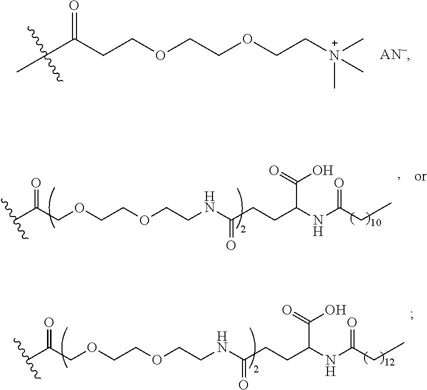

R³ is H, —CH₃, —CF₃, or

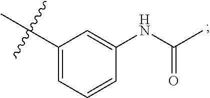 [SMILES: *c1cccc(NC(C)=O)c1]

R⁴ is H, or —CH₃;
X8

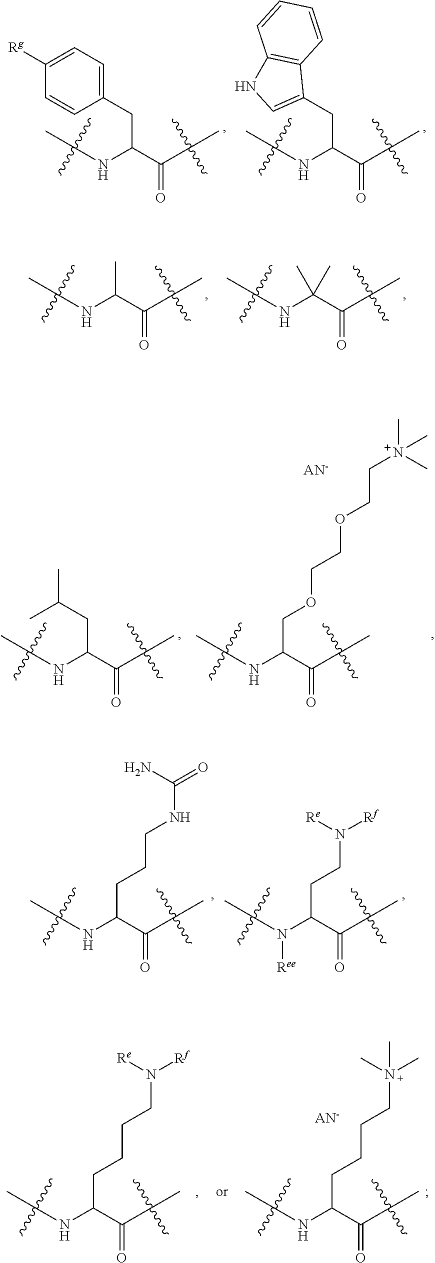

wherein:
   R^{e} is H, or —CH₃;
   R^{ee} is H;
   R^{f} is H, —CH₃, —C(CH₃)₃, —C(O)CH₃, —C(O)(CH₂CH₂O)₆CH₃, —(CH₂CH₂O)₆CH₃,

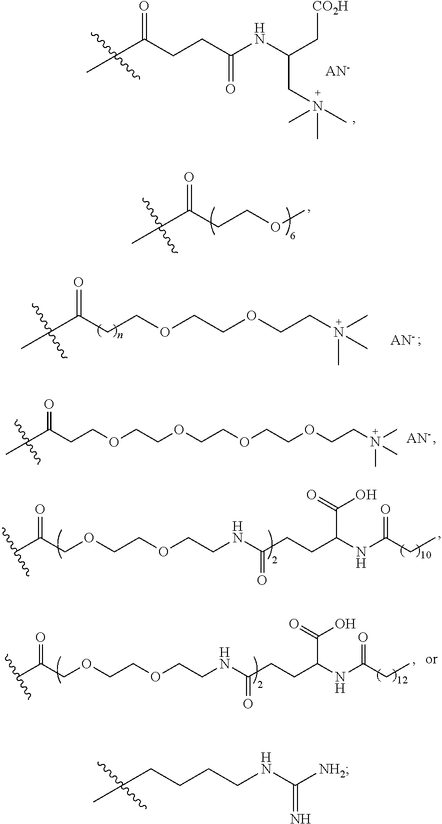

R^{g} is H, —OH, —C(O)NH₂, or —CH₂NHC(O)CH₃;
n is 0, or 1;
X12 is absent,

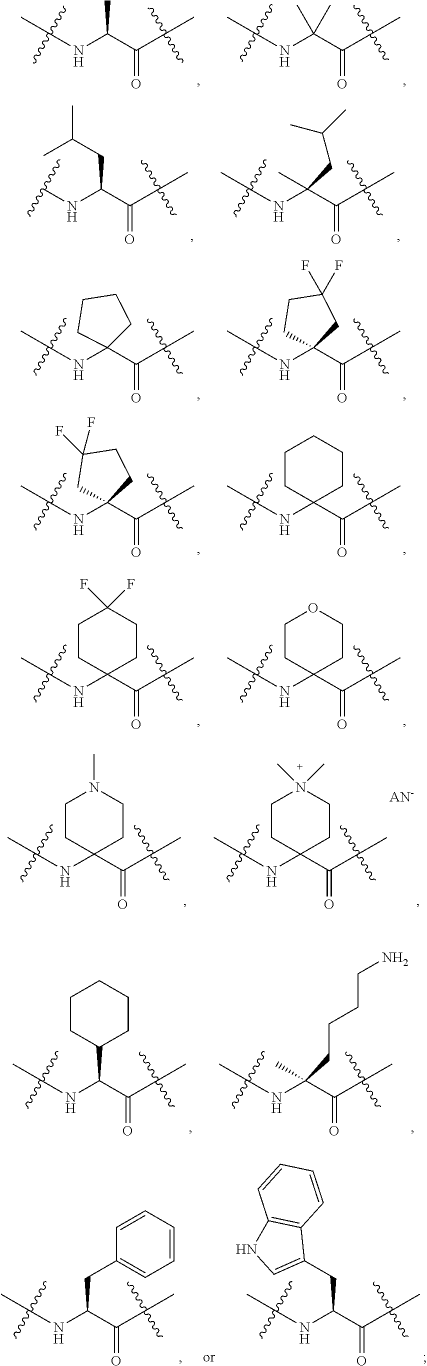

R⁶ is —CH₃, —CH₂CH₂C(O)R^{m}, —(CH₂)ₛNRⁿR^{o},

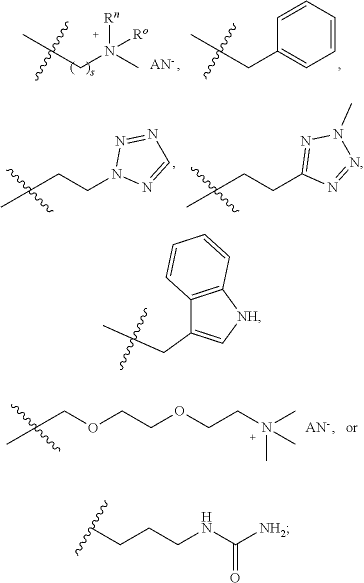

R⁷ is H, or —CH₃;
wherein:
   R^{m} is —OH, or

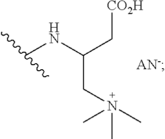

Rⁿ is H, —CH₃, —CH₂CH(CH₃)₂, —C(O)CH₃, —C(O)(CH₂CH₂O)₆CH₃, —(CH₂CH₂O)₆CH₃, —C(O)(CH₂CH₂O)ₜCH₂CH₂R^{p},

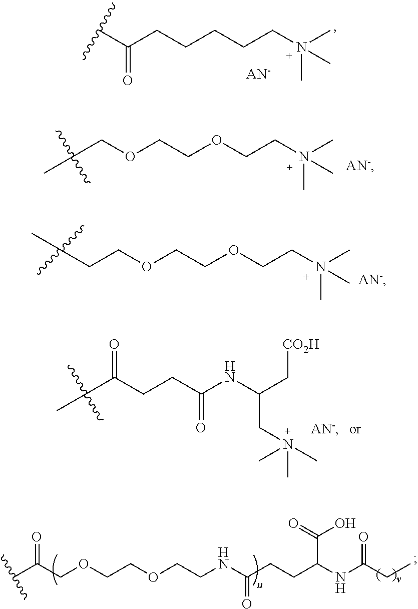

R^{o} is H, —CH₃, or —C(O)CH₃;
wherein:
   R^{p} is NHC(O)CH₃, or

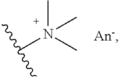

s is 1, 2, or 4;
t is 2, 6, 9, or 12;
u is 2 or 3;
v is 10 or 12;
R⁸ is —CH₂CH₂CH₂NHC(O)CH₃, —C(O)NH₂, or —C(O)NHCH₃;
X15 is absent,

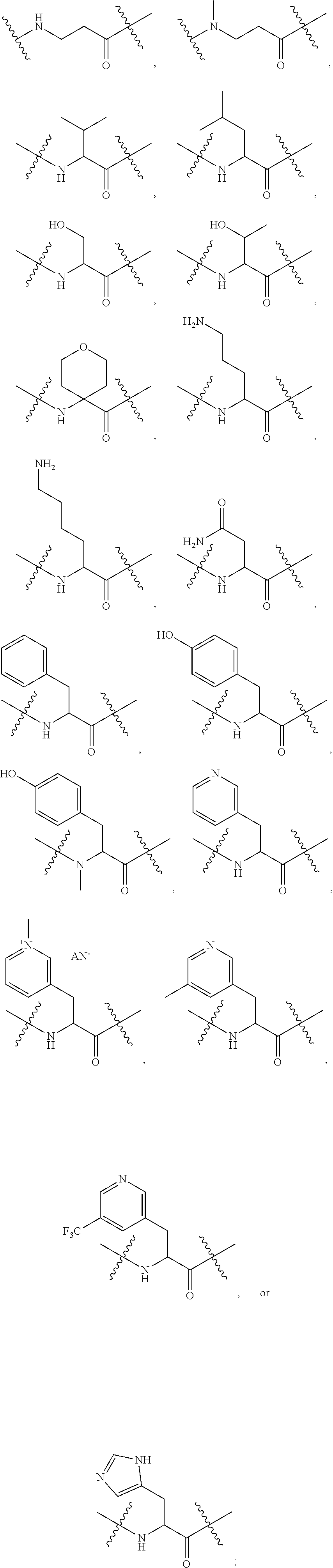

X16 is absent,

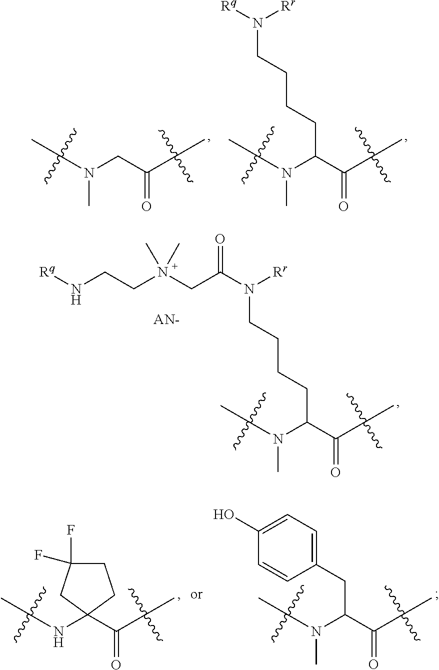

wherein:
R^{q} is

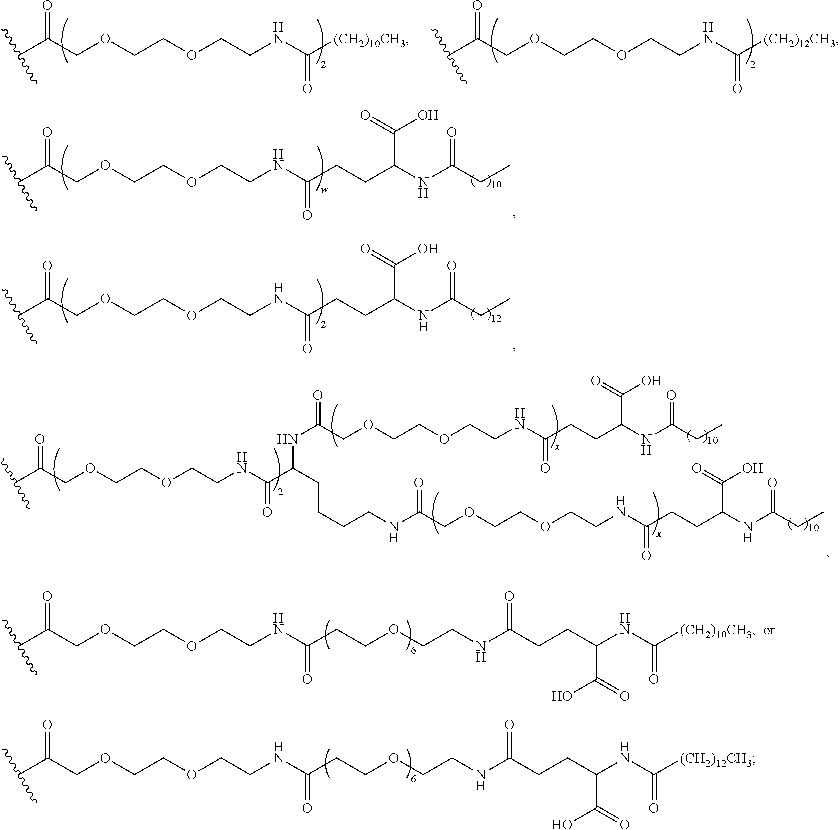

R^{r} is H;
w is 2 4, or 5;
x is 2 or 4; and
R⁹ is —NH₂, —NHCH₃, —N(CH₃)₂, —NH(CH₂CH₂O)₂CH₃, —N(CH₃)(CH₂CH₂O)₂CH₃, —NH(CH₂CH₂O)_{y}R^{s},

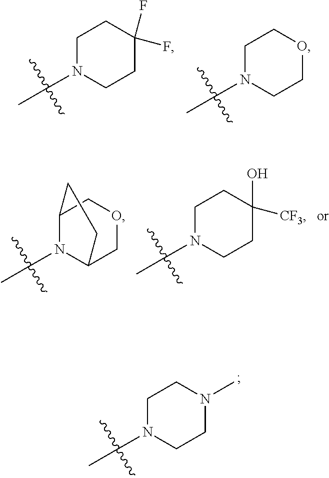

wherein:
   y is 2, 4, or 6;
R^{s} is —CH₃, or

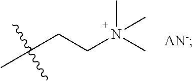

wherein:
   AN⁻as above defined for A, R, X3, X12, R1, R2, R5, R6, R8, R15, R16 and R^{a}, R^{b}, R^{d}, R^{f}, R^{h}, Rⁱ, R^{j}, R^{k}, R^{m}, Rⁿ, R^{o}, R^{s} is acetate, adipate, benzoate, benzenesulfonate, citrate, decanoate, chloride, lactate, maleate, methanesulfonate, oxalate, pivalate, propionate, succinate, sulfate, tartrate, or trifluoroacetate; or
a pharmaceutically acceptable salt thereof.

### Clause 12

12. The compound of clause 10 wherein:
AN⁻is chloride;
or a pharmaceutically acceptable salt thereof.

### Clause 13

13. The compound of clause 10 selected from
cPEG3aCO-Pen(3)—N(N(Me)2)-T-7MeW—K(NMeAc)-Pen(3)-4DMPEF-2Nal-THP-K(NMeAc)—N-3Pya-Sar-CON(Me)2;
cPEG3aCO-Pen(3)—N(N(Me)2)-T-7MeW—K(NMeAc)-Pen(3)-4DMPzEF-2Nal-THP-K(NMeAc)—N-3Pya-Sar-CON(Me)2;
cPEG3aCO-Pen(3)—N(N(Me)2)-T-7MeW—K(NMeAc)-Pen(3)-ACHMF-2Nal-THP-K(NMeAc)—N-3Pya-Sar-CON(Me)2;
cPEG3aCO-Pen(3)—N(N(Me)2)-T-7MeW—K(NMeAc)-Pen(3)-ACHMF-2Nal-THP-K(NMeAc)—N-3Pya-Sar-CON(Me)2;
cPEG3aCO-Pen(3)—N-T-7MeW—K(Ac)-Pen(3)-AEF-2Nal-THP-E-N-3Pya-Sar-CONH2;
cPEG3aCO-Pen(3)—N-T-7MeW—K(Ac)-Pen(3)-AEF-2Nal-THP—K(Ac)—N-3Pya-Sar-CONH2;
cPEG3aCO-Pen(3)—N-T-7MeW-Q(N(Me)2)-Pen(3)-AEF-2Nal-THP-Q(N(Me)2)—N-3Pya-Sar-CONH2;
cPEG3aCO-Pen(3)—N-T-7MeW-Q(N(Me)2)-Pen(3)-AEF-2Nal-THP-Q(N(Me)2)—N-3Pya-Sar-CON(Me)2;
cPEG3aCO-Pen(3)—N-T-7MeW—K(NMeAc)-Pen(3)-AEF-2Nal-THP-E-N-3Pya-Sar-CONH2;
cPEG3aCO-Pen(3)—N-T-7MeW—K(NMeAc)-Pen(3)-AEF-2Nal-THP—K(Ac)—N-3Pya-Sar-CONH2;
cPEG3aCO-Pen(3)—N-T-7MeW—K(NMeAc)-Pen(3)-AEF-2Nal-THP-K(NMeAc)—N-3Pya-Sar-CONH2;
cPEG3aCO-Pen(3)—N-T-7MeW-Q(N(Me)2)-Pen(3)-AEF-2Nal-THP-K(NMeAc)—N-3Pya-Sar-CONH2;
cPEG3aCO-Pen(3)—N-T-7MeW—K(NMeAc)-Pen(3)-AEF-2Nal-THP-Dab(NMeAc)—N-3Pya-Sar-CONH2;
cPEG3aCO-Pen(3)—N-T-7MeW-Q(N(Me)2)-Pen(3)-AEF-2Nal-THP-Dab(NMeAc)—N-3Pya-Sar-CONH2;
cPEG3aCO-Pen(3)—N-T-7(3NAcPh)W—K(Ac)-Pen(3)-AEF-2Nal-THP-E-N-3Pya-Sar-CONH2;
cPEG3aCO-Pen(3)—N-T-7(3NAcPh)W—K(Ac)-Pen(3)-AEF-2Nal-THP—K(Ac)—N-3Pya-Sar-CONH2;
cPEG3aCO-r-Pen(3)—N-T-7MeW—K(Ac)-Pen(3)-AEF-2Nal-THP-E-N-5MePyridinAla-Sar-CONH2;
cPEG3aCO-Pen(3)—N(N(Me)2)-T-7MeW-Q(N(Me)2)-Pen(3)-AEF-2Nal-THP-Q(N(Me)2)—N-3Pya-Sar-CON(Me)2;
cPEG3aCO-Pen(3)—N(N(Me)2)-T-7MeW-AIB-Pen(3)-AEF-2Nal-THP-AIB-N-3Pya-Sar-CON(Me)2;
cPEG3aCO-Pen(3)—N(N(Me)2)-T-7MeW-Q(N(Me)2)-Pen(3)-AEF-2Nal-THP-Q(N(Me)2)—N-3Pya-Sar-CONH2;
cPEG3aCO-Pen(3)—N(N(Me)2)-T-7MeW—K(NMeAc)-Pen(3)-AEF-2Nal-THP—K(Ac)—N-3Pya-Sar-CONH2;
cPEG3aCO-Pen(3)—N(N(Me)2)-T-7MeW—K(NMeAc)-Pen(3)-AEF-2Nal-THP-Q(N(Me)2)—N-3Pya-Sar-CONH2;
cPEG3aCO-Pen(3)—N(N(Me)2)-T-7MeW-Q(N(Me)2)-Pen(3)-AEF-2Nal-THP-Dab(NMeAc)—N-3Pya-Sar-CON(Me)2;
cPEG3aCO-Pen(3)—N(N(Me)2)-T-7MeW-Dab(NMeAc)-Pen(3)-AEF-2Nal-THP-Dab(NMeAc)—N-3Pya-Sar-CON(Me)2;
cPEG3aCO-Pen(3)—N(N(Me)2)-T-7MeW—K(NMeAc)-Pen(3)-AEF-2Nal-THP-K(NMeAc)—N-3Pya-Sar-CONH2;
cPEG3aCO-Pen(3)—N(N(Me)2)-T-7MeW-Dab(NMeAc)-Pen(3)-AEF-2Nal-THP-K(NMeAc)—N-3Pya-Sar-CONH2;
cPEG3aCO-Pen(3)—N(N(Me)2)-T-7MeW-Dab(NMeAc)-Pen(3)-AEF-2Nal-THP-E-N-3Pya-Sar-CONH2;
cPEG3aCO-Pen(3)—N(N(Me)2)-T-7MeW—K(NMeAc)-Pen(3)-AEF-2Nal-THP-Dab(NMeAc)—N-3Pya-Sar-CON(Me)2;
cPEG3aCO-Pen(3)—N(N(Me)2)-T-7MeW—K(NMeAc)-Pen(3)-AEF-2Nal-diFCpx-Dab(NMeAc)—N-3Pya-Sar-CON(Me)2;
cPEG3aCO-Pen(3)—N(N(Me)2)-T-7MeW—K(NMeAc)-Pen(3)-AEF-2Nal-diFCpx-Dab(NMeAc)—N-3Pya-Sar-CON(Me)2;
cPEG3aCO-Pen(3)—N(N(Me)2)-T-7MeW-Dab(NMeAc)-Pen(3)-AEF-2Nal-THP-Dab(NMeAc)—N-3Pya-Sar-CONH(PEG3a);
cPEG3aCO-Pen(3)—N(N(Me)2)-T-7MeW-Dab(NMecPEG3a)-Pen(3)-AEF-2Nal-THP-Dab(NMeAc)—N-3Pya-Sar-CON(Me)2;
cPEG3aCO-Pen(3)—N(N(Me)2)-T-7MeW-APEG2Ser-Pen(3)-AEF-2Nal-THP-K(NMeAc)—N-3Pya-Sar-CON(Me)2;
cPEG3aCO-Pen(3)—N(N(Me)2)-T-7MeW-APEG2Ser(R*)-Pen(3)-AEF-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF((Ch)cPEG3a)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(AcCh)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(aPEG2a)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(BisMEP))-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(BisMEPa)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(BisPEG2a)(RS)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(BisPEG2a)(S*)-2Nal-THP-K(NMeAc)—N-3Pya-Sar-CON(Me)2;
cPEG3aCO-Pen(3)—N-T-7MeW—K(Ac)-Pen(3)-AEF(G)-2Nal-THP—K(Ac)—N-3Pya-Sar-CONH2;
cPEG3aCO-Pen(3)—N(N(Me)2)-T-7MeW—K(NMeAc)-Pen(3)-AEF(MePrpa)-2Nal-THP-K(NMeAc)—N-3Pya-Sar-CON(Me)2;
cPEG3aCO-Pen(3)—N(N(Me)2)-T-7MeW-Q(N(Me)2)-Pen(3)-AEF(N(Me)2)-2Nal-THP-Q(N(Me)2)—N-3Pya-Sar-CON(Me)2;
cPEG3aCO-Pen(3)—N(N(Me)2)-T-7MeW—K(NMeAc)-Pen(3)-AEF(N(Me)2)-2Nal-THP-K(NMeAc)—N-3Pya-Sar-CON(Me)2;
cPEG3aCO-Pen(3)—N(NMe2)-T-7MeW—K(NMeAc)-Pen(3)-AEF(NHCh)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(NMe2mPEG3)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(NMe3)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(NMeBismPEG3)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(NMePEG2a)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(NsCh)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(PEG2a)-2Nal-THP-K(NMeAc)—N-3Pya-Sar-CON(Me)2;
cPEG3aCO-Pen(3)—N(N(Me)2)-T-7MeW—K(NMeAc)-Pen(3)-AEF(SPD)-2Nal-THP-K(NMeAc)—N-3Pya-Sar-CON(Me)2;
cPEG3aCO-Pen(3)—N(N(Me)2)-T-7MeW—K(NMeAc)-Pen(3)-APEG2F-2Nal-THP-K(NMeAc)—N-3Pya-Sar-CON(Me)2;
cPEG3aCO-Pen(3)-A-T-7MeW-A-Pen(3)-APEG3F-2Nal-A-K(NMeAc)—N-3Pya-Sar-CON(Me)2;
cPEG3aCO-Pen(3)-APEG2Ser(S*)-T-7MeW—K(NMeAc)-Pen(3)-APEG3F-2Nal-THP-K(NMeAc)—N-3Pya-Sar-CON(Me)2;
cPEG3aCO-Pen(3)-Dab(Me)₃-T-7MeW—K(NMeAc)-Pen(3)-APEG3F-2Nal-THP-K(NMeAc)—N-3Pya-Sar-CON(Me)2;
cPEG3aCO-Pen(3)—F-T-7MeW—F-Pen(3)-APEG3F-2Nal-F—K(NMeAc)—N-3Pya-Sar-CON(Me)2;
cPEG3aCO-Pen(3)-Gab-T-7MeW—K(NMeAc)-Pen(3)-APEG3F-2Nal-THP-K(NMeAc)—N-3Pya-Sar-CON(Me)2;
cPEG3aCO-Pen(3)-L-T-7MeW-L-Pen(3)-APEG3F-2Nal-L-K(NMeAc)—N-3Pya-Sar-CON(Me)2;
cPEG3aCO-Pen(3)—N-T-7MeW—K(Ac)-Pen(3)-APEG3F-2Nal-THP—K(Ac)—N-3Pya-Sar-CONH2;
cPEG3aCO-Pen(3)—N-T-7MeW—K(NMeAc)-Pen(3)-APEG3F-2Nal-THP-K(NMeAc)—N-3Pya-Sar-CONH2;
cPEG3aCO-Pen(3)—N-T-7MeW—K(NMeAc)-Pen(3)-APEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-APEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-APEG3F-2Nal-THP-E-N-3Pya-Sar-CON(Me)2;
cPEG3aCO-Pen(3)—N(N(Me)2)-T-7MeW—K(NMeAc)-Pen(3)-APEG3F-2Nal-THP-Dab(NMeAc)—N-3Pya-Sar-CON(Me)2;
cPEG3aCO-Pen(3)—N(N(Me)2)-T-7MeW—K(NMeAc)-Pen(3)-APEG3F-2Nal-THP-Dab(NMeAc)—N-3Pya-Sar-CON(Me)2;
cPEG3aCO-Pen(3)—N(N(Me)2)-T-7MeW—K(NMeAc)-Pen(3)-APEG3F-2Nal-THP—K(Ac)—N-3Pya-Sar-CON(Me)2;
cPEG3aCO-Pen(3)—N(N(Me)2)-T-7MeW—K(Ac)-Pen(3)-APEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-APEG3F-2Nal-THP-K(NMeAc)—N-3Pya-Sar-CONH2;
cPEG3aCO-Pen(3)—N(N(Me)2)-T-7MeW—K(NMeAc)-Pen(3)-APEG3F-2Nal-THP-Dap(NMeAc)—N-3Pya-Sar-CON(Me)2;
cPEG3aCO-Pen(3)—N(N(Me)2)-T-7MeW—K(NMeAc)-Pen(3)-APEG3F-2Nal-THP-A-N-3Pya-Sar-CON(Me)2;
cPEG3aCO-Pen(3)—N(N(Me)2)-T-7MeW—K(NMeAc)-Pen(3)-APEG3F-2Nal-THP-L-N-3Pya-Sar-CON(Me)2;
cPEG3aCO-Pen(3)—N(N(Me)2)-T-7MeW—K(NMeAc)-Pen(3)-APEG3F-2Nal-THP—F—N-3Pya-Sar-CON(Me)2;
cPEG3aCO-Pen(3)—N(N(Me)2)-T-7MeW—K(NMeAc)-Pen(3)-APEG3F-2Nal-THP—W—N-3Pya-Sar-CON(Me)2;
cPEG3aCO-Pen(3)—W-T-7MeW—W-Pen(3)-APEG3F-2Nal-W—K(NMeAc)—N-3Pya-Sar-CON(Me)2;
cPEG3aCO-Pen(3)—N(N(Me)2)-T-7MeW—K(NMeAc)-Pen(3)-dFPPEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-Diazabiclyclooctane6F-2Nal-THP-K(NMeAc)—N-3Pya-Sar-CON(Me)2;
cPEG3aCO-Pen(3)—N(N(Me)2)-T-7MeW—K(NMeAc)-Pen(3)-DMMMF-2Nal-THP-K(NMeAc)—N-3Pya-Sar-CON(Me)2;
cPEG3aCO-Pen(3)—N(N(Me)2)-T-7MeW—K(NMeAc)-Pen(3)-DMPMF-2Nal-THP-K(NMeAc)—N-3Pya-Sar-CON(Me)2;
cPEG3aCO-Pen(3)—N(N(Me)2)-T-7MeW—K(NMeAc)-Pen(3)-DMTASF-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4G)-2Nal-THP-K(NMeAc)—N-3Pya-Sar-CON(Me)2;
cPEG3aCO-Pen(3)—N-T-7MeW—K(Ac)-Pen(3)—F(4N3)-2Nal-THP—K(Ac)—N-3Pya-Sar-CONH2;
cPEG3aCO-Pen(3)—N(N(Me)2)-T-7MeW—K(NMeAc)-Pen(3)—F(4N3)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4Tz1DMA4mPEG)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4Tz1MME)-2Nal-THP-K(NMeAc)—N-3Pya-Sar-CON(Me)2;
cPEG3aCO-Pen(3)—N-T-7MeW—K(Ac)-Pen(3)—F(4Tz1MMo3)-2Nal-THP—K(Ac)—N-3Pya-Sar-CONH2;
cPEG3aCO-Pen(3)—N(N(Me)2)-T-7MeW—K(NMeAc)-Pen(3)—F(4Tz1MMo4)-2Nal-THP-K(NMeAc)—N-3Pya-Sar-CON(Me)2;
cPEG3aCO-Pen(3)—N-T-7MeW—K(Ac)-Pen(3)—F(4Tz1TMA1)-2Nal-THP—K(Ac)—N-3Pya-Sar-CONH2;
cPEG3aCO-Pen(3)—N(N(Me)2)-T-7MeW—K(NMeAc)-Pen(3)—F(4Tz1TMA1)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4Tz1TMA2)-2Nal-THP-K(NMeAc)—N-3Pya-Sar-CON(Me)2;
cPEG3aCO-Pen(3)—N-T-7MeW—K(Ac)-Pen(3)—F(4Tz1TMA3)-2Nal-THP—K(Ac)—N-3Pya-Sar-CONH2;
cPEG3aCO-Pen(3)—N(N(Me)2)-T-7MeW—K(NMeAc)-Pen(3)—F(4Tz1TMA3)-2Nal-THP-K(NMeAc)—N-3Pya-Sar-CON(Me)2;
cPEG3aCO-Pen(3)—N-T-7MeW—K(Ac)-Pen(3)—F(4Tz1TMA4)-2Nal-THP—K(Ac)—N-3Pya-Sar-CONH2;
cPEG3aCO-Pen(3)—N(N(Me)2)-T-7MeW—K(NMeAc)-Pen(3)—F(4Tz1TMA4)-2Nal-THP-K(NMeAc)—N-3Pya-Sar-CON(Me)2;
cPEG3aCO-Pen(3)—N(N(Me)2)-T-7MeW—K(NMeAc)-Pen(3)—F(4Tz1TMA5)-2Nal-THP-K(NMeAc)—N-3Pya-Sar-CON(Me)2;
cPEG3aCO-Pen(3)—N(N(Me)2)-T-7MeW—K(NMeAc)-Pen(3)-GPEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-hFTMAPF-2Nal-THP-K(NMeAc)—N-3Pya-Sar-CON(Me)2;
cPEG3aCO-Pen(3)—N(N(Me)2)-T-7MeW—K(NMeAc)-Pen(3)-MMoEF-2Nal-THP-K(NMeAc)—N-3Pya-Sar-CON(Me)2;
cPEG3aCO-Pen(3)—N(N(Me)2)-T-7(3NAcPh)W—K(NMeAc)-Pen(3)-MMoEF-2Nal-THP-K(NMeAc)—N-3Pya-Sar-CON(Me)2;
cPEG3aCO-Pen(3)—N(N(Me)2)-T-7MeW—K(NMeAc)-Pen(3)-MMoPF-2Nal-THP-K(NMeAc)—N-3Pya-Sar-CON(Me)2;
cPEG3aCO-Pen(3)—N(N(Me)2)-T-7MeW—K(NMeAc)-Pen(3)-MMPEG3F-2Nal-THIP-K(NMeAc)—N-3Pya-Sar-CON(Me)2;
cPEG3aCO-Pen(3)—N(N(Me)2)-T-7MeW—K(NMeAc)-Pen(3)-morfTMA4F-2Nal-THIP-K(NMeAc)—N-3Pya-Sar-CON(Me)2;
cPEG3aCO-Pen(3)—N(N(Me)2)-T-7MeW—K(NMeAc)-Pen(3)-mPEG2TMA2F-2Nal-THIP-K(NMeAc)—N-3Pya-Sar-CON(Me)2;
cPEG3aCO-Pen(3)—N(N(Me)2)-T-7MeW—K(NMeAc)-Pen(3)-mPEG2TMA4F-2Nal-THIP-K(NMeAc)—N-3Pya-Sar-CON(Me)2;
cPEG3aCO-Pen(3)—N(N(Me)2)-T-7MeW—K(NMeAc)-Pen(3)-mPEG3 TMA4F-2Nal-THP-K(NMeAc)—N-3Pya-Sar-CON(Me)2;
cPEG3aCO-Pen(3)—N(N(Me)2)-T-7MeW—K(NMeAc)-Pen(3)-MPzPEG3F-2Nal-THIP-K(NMeAc)—N-3Pya-Sar-CON(Me)2;
cPEG3aCO-Pen(3)—N(N(Me)2)-T-7MeW—K(NMeAc)-Pen(3)-MTASF-2Nal-THP-K(NMeAc)—N-3Pya-Sar-CON(Me)2;
cPEG3aCO-Pen(3)—N(N(Me)2)-T-7MeW—K(NMeAc)-Pen(3)-NPyEF-2Nal-THIP-K(NMeAc)—N-3Pya-Sar-CON(Me)2;
cPEG3aCO-Pen(3)—N(N(Me)2)-T-7MeW—K(NMeAc)-Pen(3)-NPyPEG3F-2Nal-THIP-K(NMeAc)—N-3Pya-Sar-CON(Me)2;
cPEG3aCO-Pen(3)—N(N(Me)2)-T-7MeW—K(NMeAc)-Pen(3)-PiperazinequatF-2Nal-THP-K(NMeAc)—N-3Pya-Sar-CON(Me)2;
cPEG3aCO-Pen(3)—N(N(Me)2)-T-7MeW—K(NMeAc)-Pen(3)-TBAPEG3F-2Nal-THP-K(NMeAc)—N-3Pya-Sar-CON(Me)2;
cPEG3aCO-Pen(3)—N(N(Me)2)-T-7MeW—K(NMeAc)-Pen(3)-TMA3F-2Nal-THP-K(NMeAc)—N-3Pya-Sar-CON(Me)2;
cPEG3aCO-Pen(3)—N(N(Me)2)-T-7MeW—K(NMeAc)-Pen(3)-TMA3F-2Nal-THP-K(NMeAc)—N-3Pya-Sar-CON(Me)2;
cPEG3aCO-Pen(3)—N(N(Me)2)-T-7MeW—K(NMeAc)-Pen(3)-TMA4F-2Nal-THP-K(NMeAc)—N-3Pya-Sar-CON(Me)2;
cPEG3aCO-Pen(3)—N(N(Me)2)-T-7MeW—K(NMeAc)-Pen(3)-TMA6F-2Nal-THP-K(NMeAc)—N-3Pya-Sar-CON(Me)2;
cPEG3aCO-Pen(3)—N(N(Me)2)-T-7MeW—K(NMeAc)-Pen(3)-TMA8F-2Nal-THP-K(NMeAc)—N-3Pya-Sar-CON(Me)2;
cPEG3aCO-Pen(3)—K(cPEG3a)-T-7MeW—K(NMeAc)-Pen(3)-TMAPF-2Nal-THP-K(NMeAc)—N-3Pya-Sar-CON(Me)2;
cPEG3aCO-Pen(3)—K(Me)₃-T-7MeW—K(NMeAc)-Pen(3)-TMAPF-2Nal-THP-K(NMeAc)—N-3Pya-Sar-CON(Me)2;
cPEG3aCO-Pen(3)—K(Me)₃-T-7MeW—K(NMeAc)-Pen(3)-TMAPF-2Nal-Achx-K(NMeAc)—N-3Pya-Sar-CON(Me)2;
cPEG3aCO-Pen(3)—K(PEG2PEG2gEC12)-T-7MeW—K(Ac)-Pen(3)-TMAPF-2Nal-THP-K(NMeAc)—N-3Pya-NMeK(PEG2PEG2gEC12)—CONH2;
cPEG3aCO-k(PEG2PEG2gEC12)-Pen(3)—K(PEG2PEG2gEC12)-T-7MeW—K(NMeAc)-Pen(3)-TMAPF-2Nal-THP-K(NMeAc)—N-3Pya-Sar-CON(Me)2;
cPEG3aCO-Pen(3)—K(PEG2PEG2gEC12)-T-7MeW—K(NMeAc)-Pen(3)-TMAPF-2Nal-THP-K(NMeAc)—N-3Pya-Sar-CON(Me)2;
cPEG3aCO-Pen(3)—K(PEG2PEG2gEC12)-T-7MeW—K(PEG2PEG2gEC12)-Pen(3)-TMAPF-2Nal-THP-K(NMeAc)—N-3Pya-Sar-CON(Me)2;
cPEG3aCO-Pen(3)—K(PEG2PEG2gEC12)-T-7MeW—K(NMeAc)-Pen(3)-TMAPF-2Nal-THP-K(PEG2PEG2gEC12)—N-3Pya-Sar-CON(Me)2;
cPEG3aCO-Pen(3)—K(PEG2PEG2gEC14)-T-7MeW—K(Ac)-Pen(3)-TMAPF-2Nal-THP-K(NMeAc)—N-3Pya-NMeK(PEG2PEG2gEC14)—CONH2;
cPEG3aCO-k(PEG2PEG2gEC14)-Pen(3)—K(PEG2PEG2gEC14)-T-7MeW—K(NMeAc)-Pen(3)-TMAPF-2Nal-THP-K(NMeAc)—N-3Pya-Sar-CON(Me)2;
cPEG3aCO-Pen(3)—K(PEG2PEG2gEC14)-T-7MeW—K(NMeAc)-Pen(3)-TMAPF-2Nal-THP-K(NMeAc)—N-3Pya-Sar-CON(Me)2;
cPEG3aCO-Pen(3)—K(PEG2PEG2gEC14)-T-7MeW—K(PEG2PEG2gEC14)-Pen(3)-TMAPF-2Nal-THP-K(NMeAc)—N-3Pya-Sar-CON(Me)2;
cPEG3aCO-Pen(3)—K(PEG2PEG2gEC14)-T-7MeW—K(NMeAc)-Pen(3)-TMAPF-2Nal-THP-K(PEG2PEG2gEC14)—N-3Pya-Sar-CON(Me)2;
cPEG3aCO-Pen(3)—N-T-7MeW—K(NMeAc)-Pen(3)-TMAPF-2Nal-THP-K(NMeAc)—N-3Pya-Sar-CONH2;
cPEG3aCO-Pen(3)—N-T-7MeW—K(NMeAc)-Pen(3)-TMAPF-2Nal-THP-K(NMeAc)—N-3Pya-Sar-CON(Me)2;
cPEG3aCO-Pen(3)—N-T-7MeW—K(NMeAC)-Pen(3)-TMAPF-2Nal-THP-Dab(NMeAc)—N-3Pya-Sar-CON(Me)2;
cPEG3aCO-Pen(3)—N-T-7MeW-Dab(NMeAc)-Pen(3)-TMAPF-2Nal-THP-K(NMeAC)—N-3Pya-Sar-CON(Me)2;
cPEG3aCO-Pen(3)—N-T-7MeW—K(Ac)-Pen(3)-TMAPF-2Nal-THP-A-N-3Pya-Sar-CON(Me)2;
cPEG3aCO-Pen(3)—N-T-7MeW—K(Ac)-Pen(3)-TMAPF-2Nal-THP-K(PEG2PEG2gEC12)—N-3Pya-NMeK(PEG2PEG2gEC12)—CONH2;
cPEG3aCO-Pen(3)—N-T-7MeW—K(Ac)-Pen(3)-TMAPF-2Nal-THP-K(NMeAc)—N-3Pya-NMeK(PEG2PEG2gEC12)—CONH2;
cPEG3aCO-Pen(3)—N-T-7MeW—K(Ac)-Pen(3)-TMAPF-2Nal-THP-E-N-3Pya-Sar-CONH2;
cPEG3aCO-Pen(3)—N-T-7MeW—K(Ac)-Pen(3)-TMAPF-2Nal-THP—K(Ac)—N-3Pya-Sar-CONH2;
cPEG3aCO-Pen(3)—N-T-7(3NAcPh)W—K(Ac)-Pen(3)-TMAPF-2Nal-THP-E-N-3Pya-Sar-CONH2;
cPEG3aCO-Pen(3)—N(N(Me))-T-7MeW—K(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-THP-E-N-3Pya-Sar-CON(Me)2;
cPEG3aCO-Pen(3)—N(N(Me)2)-T-7MeW—K(NMeAc)-Pen(3)-TMAPF-2Nal-THP-K(NMeAc)—N-3Pya-Sar-CO(NHPEG3a);
cPEG3aCO-Pen(3)—N(N(Me)2)-T-7MeW-APEG2Ser(S*)-Pen(3)-TMAPF-2Nal-THP-K(NMeAc)—N-3Pya-Sar-CON(Me)2;
cPEG3aCO-Pen(3)—N(N(Me)2)-T-7MeW-APEG2Ser(R*)-Pen(3)-TMAPF-2Nal-THP-K(NMeAc)—N-3Pya-Sar-CON(Me)2;
cPEG3aCO-Pen(3)—N(N(Me)2)-T-7(3NAcPh)W—K(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(NMeAC)-Pen(3)-TMAPF-2Nal-THP-Dab(NMeAc)—N-3Pya-Sar-CON(Me)2;
cPEG3aCO-Pen(3)—N(N(Me)2)-T-7MeW—K(NMeAC)-Pen(3)-TMAPF-2Nal-THP-K(NMeAC)—N-3Pya-NMeK(PEG2PEG2C12)—CONH2;
cPEG3aCO-Pen(3)—N(N(Me)2)-T-7MeW—K(Ac)-Pen(3)-TMAPF-2Nal-THP-Dab(NMeAc)—N-3Pya-Sar-CON(Me)2;
cPEG3aCO-Pen(3)—N(N(Me)2)-T-7MeW—K(NMeAC)-Pen(3)-TMAPF-2Nal-THP-Q(N(Me)2)—N-3Pya-Sar-CON(Me)2;
cPEG3aCO-Pen(3)—N(N(Me)2)-T-7MeW-Q(N(Me)2)-Pen(3)-TMAPF-2Nal-THP-K(NMeAC)—N-3Pya-Sar-CON(Me)2;
cPEG3aCO-Pen(3)—N(N(Me)2)-T-7MeW—K(Ac)-Pen(3)-TMAPF-2Nal-THP-K(NMeAC)—N-3Pya-NMeK(PEG2PEG2gEC12)—CONH2;
cPEG3aCO-Pen(3)—N(N(Me)2)-T-7MeW—K(NMeAC)-Pen(3)-TMAPF-2Nal-THP-K(NMeAC)—N-3Pya-NMeK(PEG2PEG2gEC12)—CONH2;
cPEG3aCO-Pen(3)—N(N(Me)2)-T-7MeW—K(NMeAC)-Pen(3)-TMAPF-2Nal-THP-K(NMeAC)—N-3Pya-NMeK(PEG2PEG2gEC14)—CONH2;
cPEG3aCO-Pen(3)—N(N(Me)2)-T-7MeW—K(NMeAc)-Pen(3)-TMAPF-2Nal-THP-A-N-3Pya-Sar-CON(Me)2;
cPEG3aCO-Pen(3)—N(N(Me)2)-T-7MeW—K(NMeAc)-Pen(3)-TMAPF-2Nal-THP-K(NMeAc)—N-3Pya-Sar-CONH2;
cPEG3aCO-Pen(3)—N(N(Me)2)-T-7MeW—K(NMeAc)-Pen(3)-TMAPF-2Nal-THP-Cit-N-3Pya-Sar-CON(Me)2;
cPEG3aCO-Pen(3)—N(N(Me)2)-T-7MeW—K(Me)₃-Pen(3)-TMAPF-2Nal-THP-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-Acpx-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-Achx-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-Chg-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-Aib-K(NMeAc)—N-3Pya-Sar-CON(Me)2;
cPEG3aCO-Pen(3)—N(N(Me)2)-T-7MeW-Cit-Pen(3)-TMAPF-2Nal-THP-K(NMeAc)—N-3Pya-Sar-CON(Me)2;
cPEG3aCO-k(PEG2PEG2gEC14)-Pen(3)—N(N(Me)2)-T-7MeW—K(Ac)-Pen(3)-TMAPF-2Nal-THP-K(NMeAc)—N-3Pya-NMeK(PEG2PEG2gEC14)—CONH2;
cPEG3aCO-k(PEG2PEG2gEC12)-Pen(3)—N(N(Me)2)-T-7MeW—K(Ac)-Pen(3)-TMAPF-2Nal-THP-K(NMeAc)—N-3Pya-NMeK(PEG2PEG2gEC12)—CONH2;
cPEG3aCO-Pen(3)—N(N(Me)2)-T-7MeW—K(Ac)-Pen(3)-TMAPF-2Nal-THP-K(PEG2PEG2gEC14)—N-3Pya-NMeK(PEG2PEG2gEC14)—CONH2;
cPEG3aCO-Pen(3)—N(N(Me)2)-T-7MeW—K(PEG2PEG2gEC12)-Pen(3)-TMAPF-2Nal-THP-K(NMeAc)—N-3Pya-NMeK(PEG2PEG2gEC12)—CONH2;
cPEG3aCO-Pen(3)—N(N(Me)2)-T-7MeW—K(Ac)-Pen(3)-TMAPF-2Nal-THP-K(PEG2PEG2gEC12)—N-3Pya-NMeK(PEG2PEG2gEC12)—CONH2;
cPEG3aCO-Pen(3)—N(N(Me)2)-T-7MeW—K(PEG2PEG2gEC14)-Pen(3)-TMAPF-2Nal-THP-K(NMeAc)—N-3Pya-NMeK(PEG2PEG2gEC14)—CONH2;
cPEG3aCO-Pen(3)—N(N(Me)2)-T-7MeW-Dab(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG2C14)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG6gEC12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG6gEC14)—CONH2;
cPEG3aCO-Pen(3)—N(N(Me)2)-T-7MeW-Dab(NMeAc)-Pen(3)-TMAPF-2Nal-THP-K(NMeAc)—N-3Pya-Sar-CONH2;
cPEG3aCO-k(PEG2PEG6gEC12)-Pen(3)—N(N(Me)2)-T-7MeW—K(Ac)-Pen(3)-TMAPF-2Nal-THP-K(NMeAc)—N-3Pya-NMeK(PEG2PEG6gEC12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG2K(PEG2PEG2gEC12)₂)—CONH2;
cPEG3aCO-Pen(3)—N(N(Me)2)-T-7MeW—K(Ac)-Pen(3)-TMAPF-2Nal-THP-K(PEG2PEG2PEG2gEC12))—N-3Pya-NMeK(PEG2PEG2PEG2gEC12)—CONH2;
cPEG3aCO-k(PEG2PEG2gEC12)-Pen(3)—N(N(Me)2)-T-7MeW—K(NMeAc)-Pen(3)-TMAPF-2Nal-THP-K(NMeAc)—N-3Pya-Sar-CON(Me)2;
cPEG3aCO-k(PEG2PEG2gEC14)-Pen(3)—N(N(Me)2)-T-7MeW—K(NMeAc)-Pen(3)-TMAPF-2Nal-THP-K(NMeAc)—N-3Pya-Sar-CON(Me)2;
cPEG3aCO-k(PEG2PEG2gEC12)-Pen(3)—N(N(Me)2)-T-7MeW—K(PEG2PEG2gEC12)-Pen(3)-TMAPF-2Nal-THP-K(NMeAc)—N-3Pya-Sar-CON(Me)2;
cPEG3aCO-Pen(3)—N(N(Me)2)-T-7MeW—K(cPEG3a)-Pen(3)-TMAPF-2Nal-THP-K(NMeAc)—N-3Pya-Sar-CON(Me)2;
cPEG3aCO-Pen(3)—N(N(Me)2)-T-7MeW—K(4cpg)-Pen(3)-TMAPF-2Nal-THP-K(NMeAc)—N-3Pya-Sar-CON(Me)2;
cPEG3aCO-k(PEG2PEG2gEC12)-Pen(3)—N(N(Me)2)-T-7MeW—K(NMeAc)-Pen(3)-TMAPF-2Nal-THP-K(PEG2PEG2gEC12)—N-3Pya-Sar-CON(Me)2;
cPEG3aCO-k(PEG2PEG2gEC14)-Pen(3)—N(N(Me)2)-T-7MeW—K(NMeAc)-Pen(3)-TMAPF-2Nal-THP-K(PEG2PEG2gEC14)—N-3Pya-Sar-CON(Me)2;
cPEG3aCO-Pen(3)—N(N(Me)2)-T-7MeW—K(PEG2PEG2gEC14)-Pen(3)-TMAPF-2Nal-THP-K(NMeAc)—N-3Pya-Sar-CON(Me)2;
cPEG3aCO-Pen(3)—N(N(Me)2)-T-7MeW—K(PEG2PEG2gEC12)-Pen(3)-TMAPF-2Nal-THP-K(PEG2PEG2gEC12)—N-3Pya-Sar-CON(Me)2;
cPEG3aCO-Pen(3)—N(N(Me)2)-T-7MeW—K(NMeAc)-Pen(3)-TMAPF-2Nal-THP-K(PEG2PEG2gEC12)—N-3Pya-Sar-CON(Me)2;
cPEG3aCO-Pen(3)—N(N(Me)2)-T-7MeW—K(NMeAc)-Pen(3)-TMAPF-2Nal-THP-K(PEG2PEG2gEC14)—N-3Pya-Sar-CON(Me)2;
cPEG3aCO-k(PEG2PEG2PEG2PEG2gEC12)-Pen(3)—N(N(Me)2)-T-7MeW—K(Ac)-Pen(3)-TMAPF-2Nal-THP-K(NMeAc)—N-3Pya-NMeK(PEG2PEG2PEG2PEG2gEC12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(SP6PEG2PEG2C12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(SP6PEG2PEG2gEC12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG2K(PEG2PEG2PEG2PEG2gEC12)₂)—CONH2;
cPEG3aCO-Pen(3)—N(N(Me)2)-T-7MeW—K(PEG2PEG2gEC14)-Pen(3)-TMAPF-2Nal-THP-K(PEG2PEG2gEC14)—N-3Pya-Sar-CON(Me)2;
cPEG3aCO-Pen(3)—N(N(Me)2)-T-7MeW-Dap(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(Ac)-Pen(3)-TMAPF-2Nal-THP-Dap(NMeAc)—N-3Pya-Sar-CON(Me)2;
cPEG3aCO-Pen(3)—N(N(Me)2)-T-7MeW—K(NMeAc)-Pen(3)-Tzl(Ch)-2Nal-THP-K(NMeAc)—N-3Pya-Sar-CON(Me)2;
cPEG3aCO-Pen(3)—N(N(Me)2)-T-7MeW—K(NMeAc)-Pen(3)-TzlChmPEG-2Nal-THP-K(NMeAc)—N-3Pya-Sar-CON(Me)2;
cPEG3aCO-Pen(3)—N(N(Me)2)-T-7MeW—K(NMeAc)-Pen(3)-TzlChmPEG3-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(C9OH)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OEOXIMECh)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OTzlCh)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OTzlChC16)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OTzlC1aC8)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OTzlChmPEG)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OTzlChmPEG3)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OTz1TMA4)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OTz1TMA4)-2Nal-THP-K(NMeAc)—N-3Pya-Sar-CON(Me)2;
cPEG3aCO-Pen(3)—N(N(Me)2)-T-7MeW—K(NMeAc)-Pen(3)—Y(OZOXIMECh)-2Nal-THP-K(NMeAc)—N-3Pya-Sar-CON(Me)2;
cPEG3aCO-Pen(3)—N(N(Me)2)-T-7MeW—K(NMeAc)-Pen(3)—YC8CO(NHPEG3a)-2Nal-THP-K(NMeAc)—N-3Pya-Sar-CON(Me)2;
cPEG3aCO-Pen(3)—N(N(Me)2)-T-7MeW—K(NMeAc)-Pen(3)-YC8COPip-2Nal-THP-K(NMeAc)—N-3Pya-Sar-CON(Me)2;
cPEG3gCO-Pen(3)—N(N(Me)2)-T-7MeW—K(NMeAc)-Pen(3)-APEG3F-2Nal-THP-K(NMeAc)—N-3Pya-Sar-CON(Me)2;
cPEG3gCO-Pen(3)—N(N(Me)2)-T-7MeW—K(NMeAc)-Pen(3)-TMAPF-2Nal-THP-K(NMeAc)—N-3Pya-Sar-CON(Me)2;
   or a pharmaceutically acceptable salt thereof.

### Clause 14

14. A compound of Formula (V)

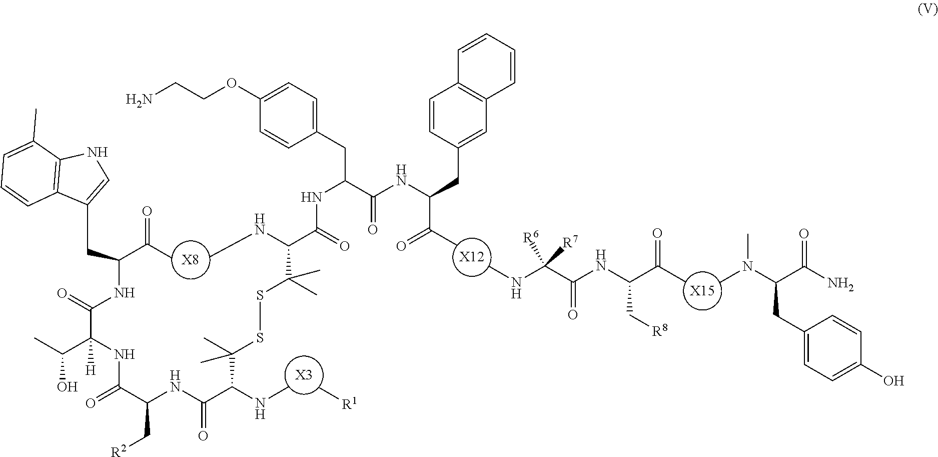

wherein:
   R¹ is —C(O)C₍₁₋₂₎alkyl, —C(O)CF₃, —COCH₂CF₃, —CO(CH₂)₅N(CH₃)₃+AN⁻, —CH₂CH₂NHC(O)CH₂O(CH₂CH₂O)₂CH₃, —CH₂CH₂NHC(O)(CH₂CH₂O)₆CH₃,

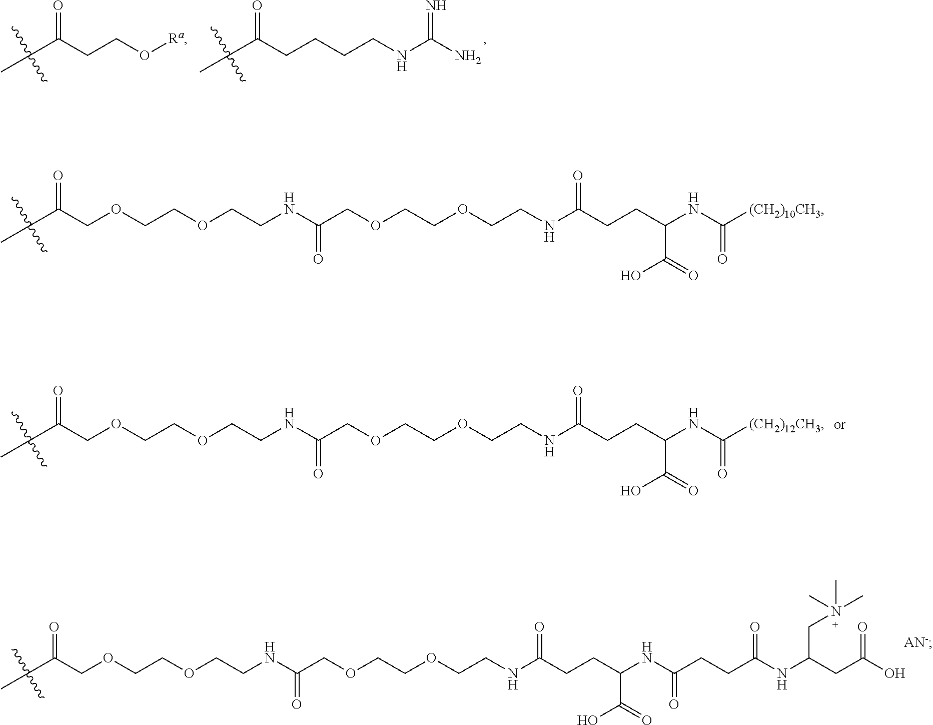

wherein:
   R^{a} is

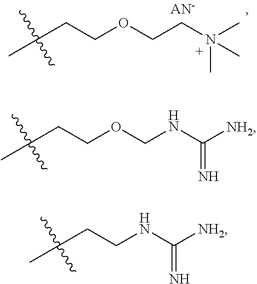

CH₂CH₂OCH₂CH₂N(CH₃)₂,
—(CH₂CH₂O)₃CH₂CH₂N(CH₃)₂, —(CH₂CH₂O)₃CH₂CH₂N(CH₃)₃ ⁺AN⁻, —(CH₂CH₂O)₅CH₂CH₂NC(O)CH₃, —(CH₂CH₂O)₈CH₂CH₂NC(O)CH₃, or
X3 is a direct bond, (D) arginine, (D) lysine substituted with R^{b},

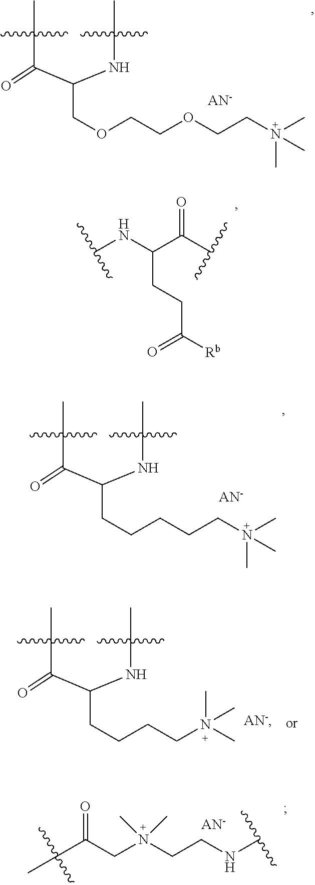

wherein:
   R^{b} is

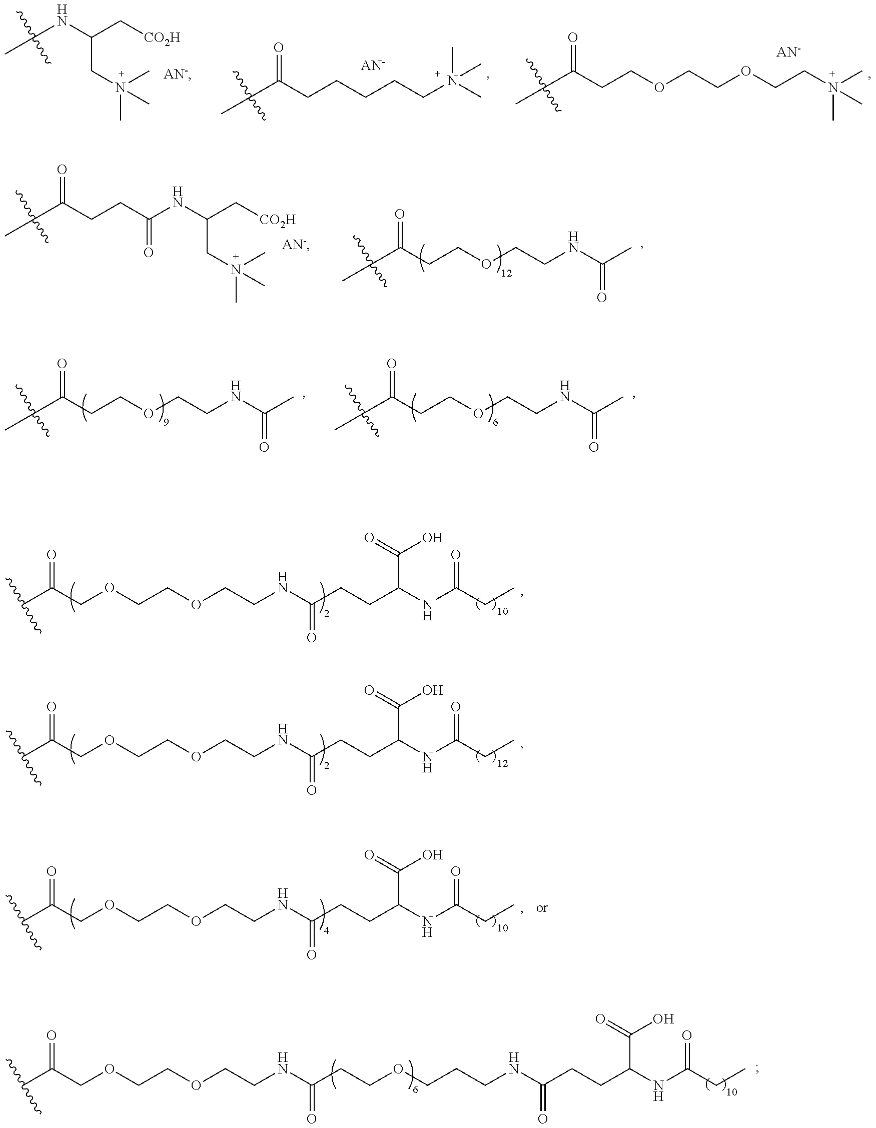

R² is H, —CH(CH₃)₂,

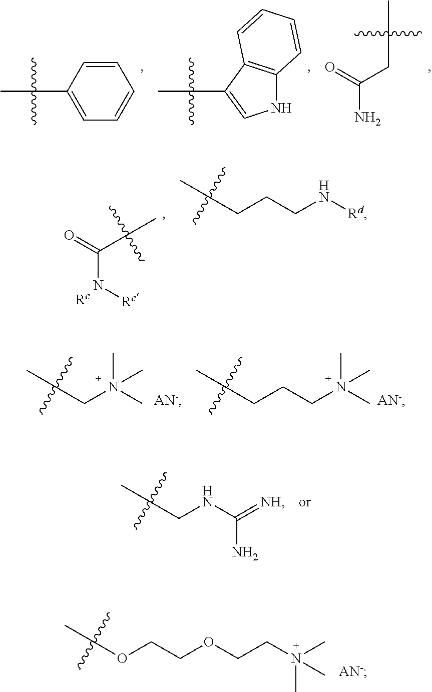

wherein:
   R^{c} is H, or —CH₃;
   R^{c}′ is H, or —CH₃;
   R^{d} is

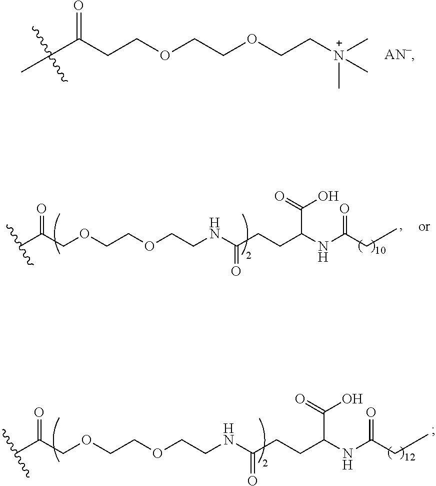

X8

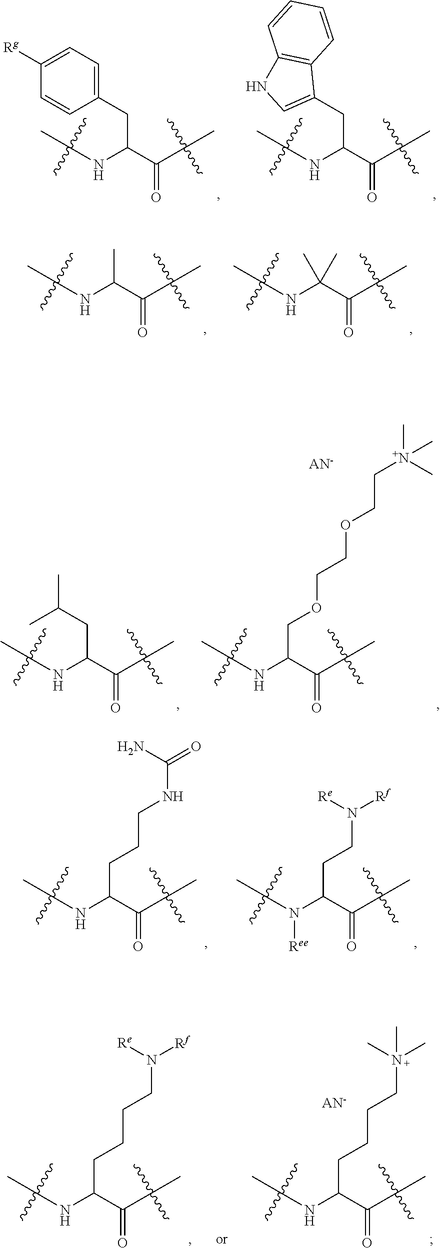

wherein:
   R^{e} is H, or —CH₃;
   R^{ee} is H;
   R^{f} is H, —CH₃, —C(CH₃)₃, —C(O)CH₃, —C(O)(CH₂CH₂O)₆CH₃, —(CH₂CH₂O)₆CH₃,

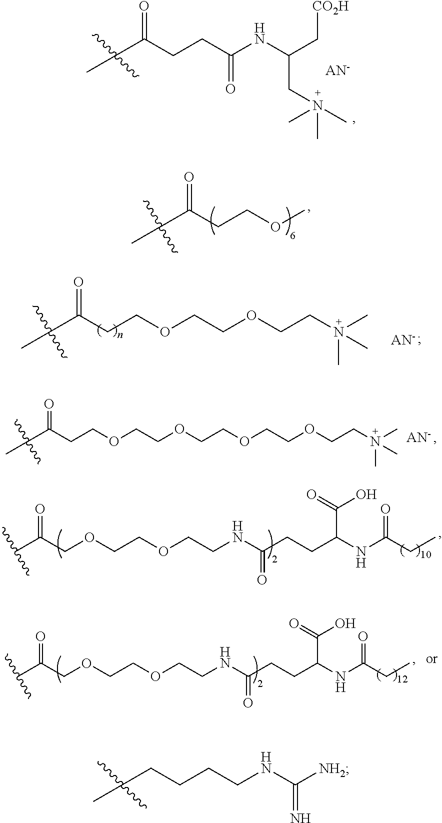

R^{g} is H, —OH, —C(O)NH₂, or —CH₂NHC(O)CH₃;
n is 0, or 1;
X12 is absent,

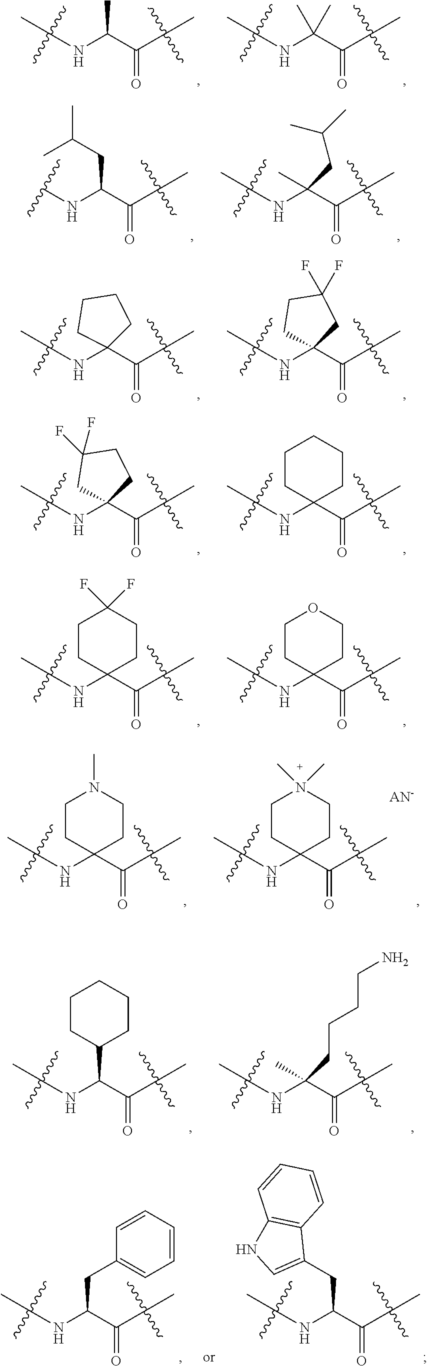

R⁶ is —CH₃, —CH₂CH₂C(O)R^{m}—(CH₂)ₛNRⁿR^{o},

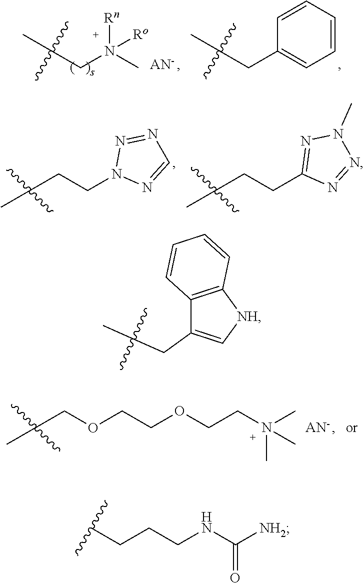

R⁷ is H, or —CH₃;
wherein:
   R^{m} is —OH, or

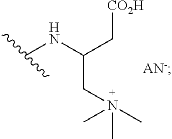

Rⁿ is H, —CH₃, —CH₂CH(CH₃)₂, —C(O)CH₃, —C(O)(CH₂CH₂O)₆CH₃, —(CH₂CH₂O)₆CH₃, —C(O)(CH₂CH₂O)ₜCH₂CH₂R^{p},

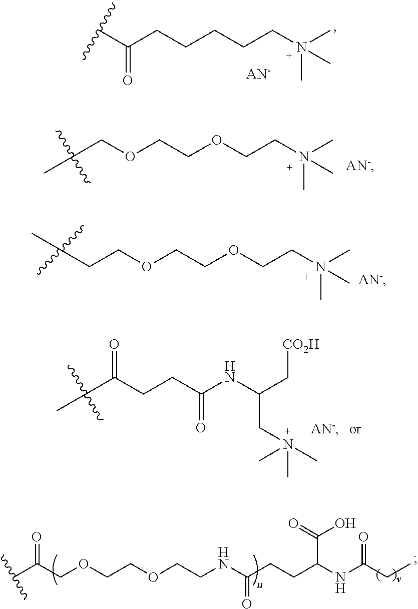

R^{o} is H, —CH₃, or —C(O)CH₃;
wherein:
   R^{p} is NHC(O)CH₃, or

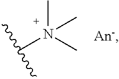

s is 1, 2, or 4;
t is 2, 6, 9, or 12;
u is 2 or 3;
v is 10 or 12;
R⁸ is —CH₂CH₂CH₂NHC(O)CH₃, —C(O)NH₂, or —C(O)NHCH₃;
X15 is absent,

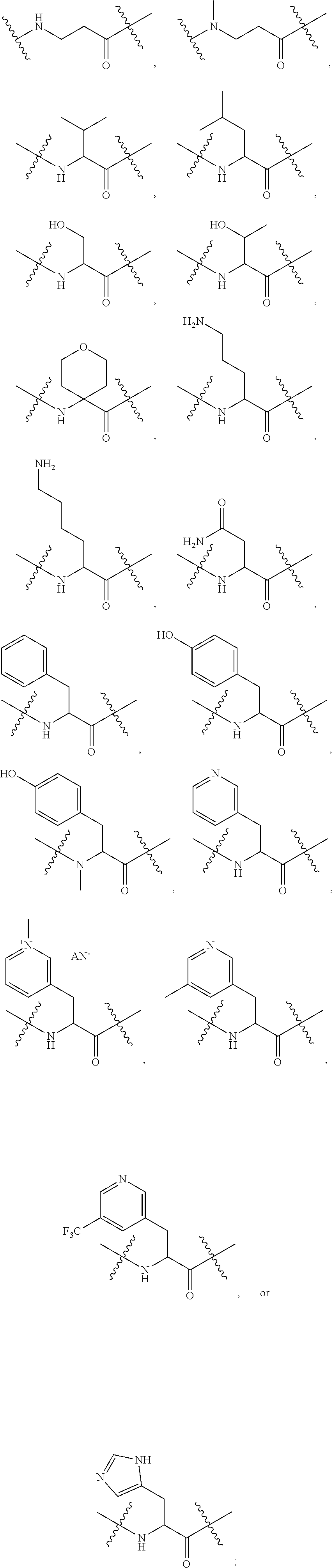

wherein:
   AN⁻as above defined for A, R, X3, X12, R1, R2, R5, R6, R8, R15, R16 and R^{a}, R^{b}, R^{d}, R^{f}, R^{h}, Rⁱ, R^{j}, R^{k}, R^{m}, Rⁿ, R^{o}, R^{s} is acetate, adipate, benzoate, benzenesulfonate, citrate, decanoate, chloride, lactate, maleate, methanesulfonate, oxalate, pivalate, propionate, succinate, sulfate, tartrate, or trifluoroacetate; or
a pharmaceutically acceptable salt thereof.

Clause 15

15. The compound of clause 14 which is
MeCO-Pen(3)—N-T-7MeW—K(Ac)-Pen(3)-AEF-2Nal-aMeK—K(Ac)—N-dK-NMeDTyr-CONH2;
MeCO-Pen(3)—N-T-7MeW—K(Ac)-Pen(3)-AEF-2Nal-aMeK—K(Ac)—N-dL-NMeDTyr-CONH2;
MeCO-Pen(3)—N-T-7MeW—K(Ac)-Pen(3)-AEF-2Nal-aMeK—K(Ac)—N—F—NMeDTyr-CONH2;
MeCO-Pen(3)—N-T-7MeW—K(Ac)-Pen(3)-AEF-2Nal-aMeK—K(Ac)—N—H—NMeDTyr-CONH2;
MeCO-r-Pen(3)—N-T-7MeW—K(Ac)-Pen(3)-AEF-2Nal-aMeK-E-N—H—NMeDTyr-CONH2;
MeCO-Pen(3)—N-T-7MeW—K(Ac)-Pen(3)-AEF-2Nal-aMeK—K(Ac)—N-Paf-NMeDTyr-CONH2;
MeCO-r-Pen(3)—N-T-7MeW—K(Ac)-Pen(3)-AEF-2Nal-THP-E-N-h-NMeDTyr-CONH2;
MeCO-r-Pen(3)—N-T-7MeW—K(Ac)-Pen(3)-AEF-2Nal-THP-E-N—N—NMeDTyr-CONH2;
MeCO-Pen(3)—N-T-7MeW—K(Ac)-Pen(3)-AEF-2Nal-aMeK—K(Ac)—N—N—NMeDTyr-CONH2;
MeCO-Pen(3)—N-T-7MeW—K(Ac)-Pen(3)-AEF-2Nal-aMeK—K(Ac)—N-v-NMeDTyr-CONH2;
MeCO-Pen(3)—N-T-7MeW—K(Ac)-Pen(3)-AEF-2Nal-aMeK—K(Ac)—N-t-NMeDTyr-CONH2;
MeCO-r-Pen(3)—N-T-7MeW-Cit-Pen(3)-AEF-2Nal-aMeL-E-N-k-NMeDTyr-CONH2;
MeCO-r-Pen(3)—N-T-7MeW-Cit-Pen(3)-AEF-2Nal-aMeL-E-N-h-NMeDTyr-CONH2;
MeCO-Pen(3)—N-T-7MeW—K(Ac)-Pen(3)-AEF-2Nal-aMeK—K(Ac)—N-orn-NMeDTyr-CONH2;
MeCO-Pen(3)—N-T-7MeW—K(Ac)-Pen(3)-AEF-2Nal-aMeK—K(Ac)—N-s-NMeDTyr-CONH2;
MeCO-Pen(3)—N-T-7MeW—K(Ac)-Pen(3)-AEF-2Nal-aMeK—K(Ac)—N-f-NMeDTyr-CONH2;
MeCO-Pen(3)—N-T-7MeW—Y-Pen(3)-AEF-2Nal-aMeK—K(Ac)—N-k-NMeDTyr-CONH2;
MeCO-Pen(3)—N-T-7MeW-4AmPhe-Pen(3)-AEF-2Nal-aMeK—K(Ac)—N-k-NMeDTyr-CONH2;
MeCO-Pen(3)—N-T-7MeW-Paf(Ac)-Pen(3)-AEF-2Nal-aMeK—K(Ac)—N-k-NMeDTyr-CONH2;
MeCO-Pen(3)—N-T-7MeW—K(Ac)-Pen(3)-AEF-2Nal-aMeK—K(Ac)—N-y-NMeDTyr-CONH2;
MeCO-Pen(3)—N-T-7MeW-Cit-Pen(3)-AEF-2Nal-aMeL-K(Ac)—N-k-NMeDTyr-CONH2;
MeCO-Pen(3)—N-T-7MeW—K(Ac)-Pen(3)-AEF-2Nal-aMeK—K(Ac)—N-h-NMeDTyr-CONH2;
MeCO-Pen(3)—N-T-7MeW—K(Ac)-Pen(3)-AEF-2Nal-aMeK—K(Ac)—N-bAla-NMeDTyr-CONH2;
MeCO-Pen(3)—N-T-7MeW—K(Ac)-Pen(3)-AEF-2Nal-aMeK—K(Ac)—N-bAla-NMeDTyr-CONH2;
MeCO-Pen(3)—N-T-7MeW—K(Ac)-Pen(3)-AEF-2Nal-aMeK—K(Ac)—N-bAla-NMeDTyr-CONH2;
MeCO-Pen(3)—N-T-7MeW—K(Ac)-Pen(3)-AEF-2Nal-aMeK—K(Ac)—N-3Pya-NMeDTyr-CONH2;
MeCO-Pen(3)—N-T-7MeW—K(Ac)-Pen(3)-AEF-2Nal-aMeK—K(Ac)—N-3pya-NMeDTyr-CONH2;
MeCO-r-Pen(3)—N-T-7MeW—K(Ac)-Pen(3)-AEF-2Nal-THP-E-N-3Pya-NMeDTyr-CONH2;
MeCO-r-Pen(3)-Q-T-W-Q-Pen(3)-AEF-2Nal-THP-aMeE-N—F—NMeDTyr-CONH2;
MeCO-r-Pen(3)-Q-T-W-Q-Pen(3)-AEF-2Nal-THP-E-N—N—NMe DTyr-CONH2;
   or a pharmaceutically acceptable salt thereof.

### Clause 16.

16. A compound of Formula (VI)

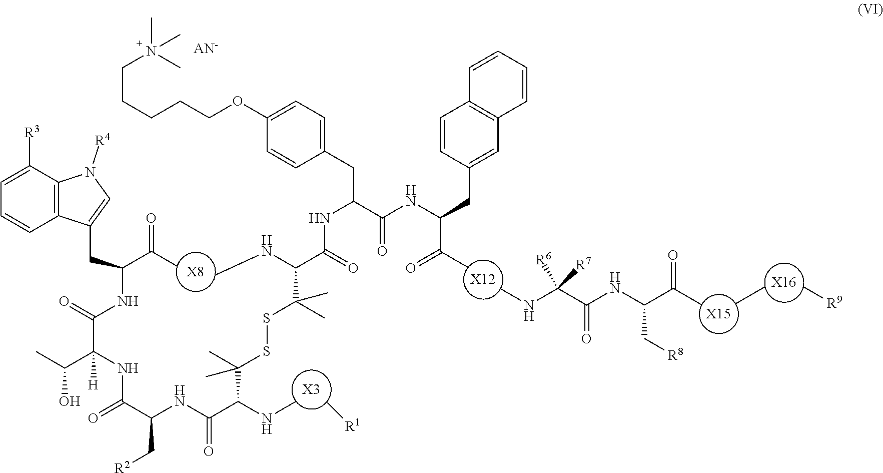

wherein:
   R¹ is —C(O)C₍₁₋₂₎alkyl, —C(O)CF₃, —COCH₂CF₃, —CO(CH₂)₅N(CH₃)₃⁺AN⁻, —CH₂CH₂NHC(O)CH₂O(CH₂CH₂O)₂CH₃, —CH₂CH₂NHC(O)(CH₂CH₂O)₆CH₃,

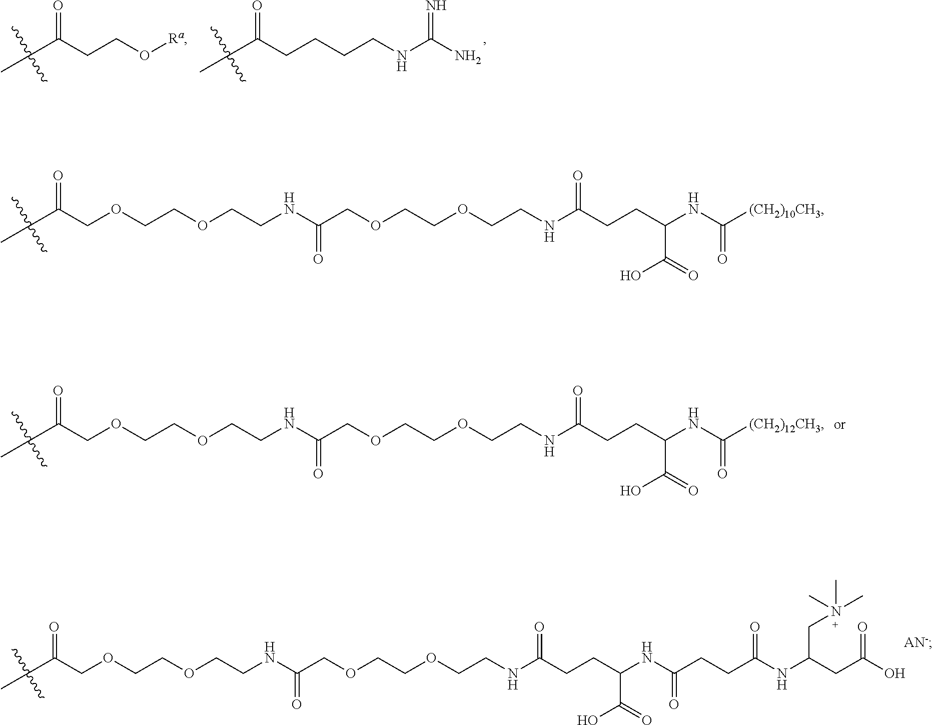

wherein:
   R^{a} is

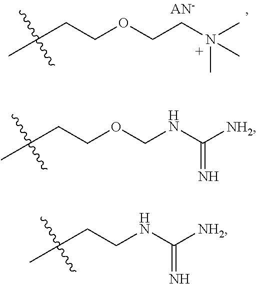

CH₂CH₂OCH₂CH₂N(CH₃)₂,
—(CH₂CH₂O)₃CH₂CH₂N(CH₃)₂, —(CH₂CH₂O)₃CH₂CH₂N(CH₃)₃ ⁺AN⁻, —(CH₂CH₂O)₅CH₂CH₂NC(O)CH₃, —(CH₂CH₂O)₈CH₂CH₂NC(O)CH₃, or —(CH₂CH₂O)₁₁CH₂CH₂NC(O)CH₃;
X3 is a direct bond, (D) arginine, (D) lysine substituted with R^{b},

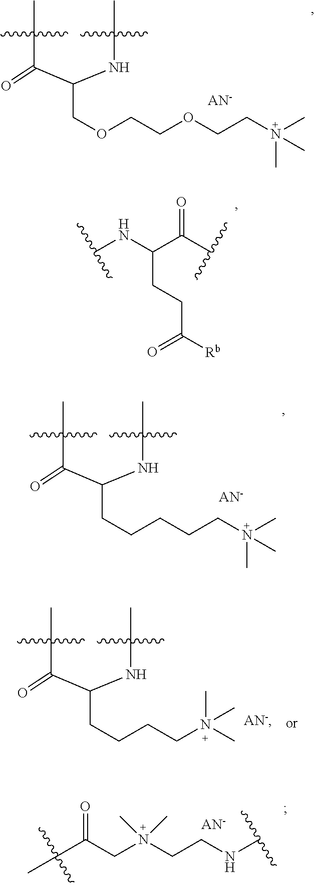

wherein
   R^{b} is

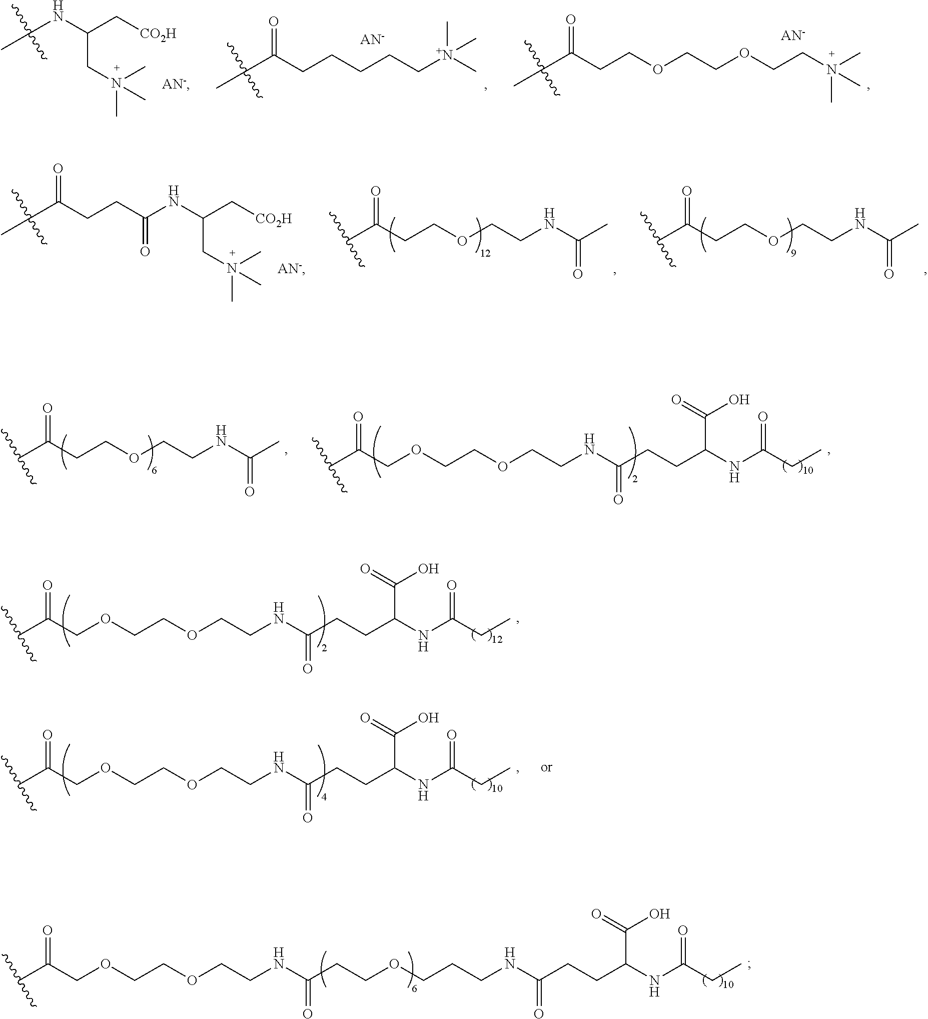

R² is H, —CH(CH₃)₂,

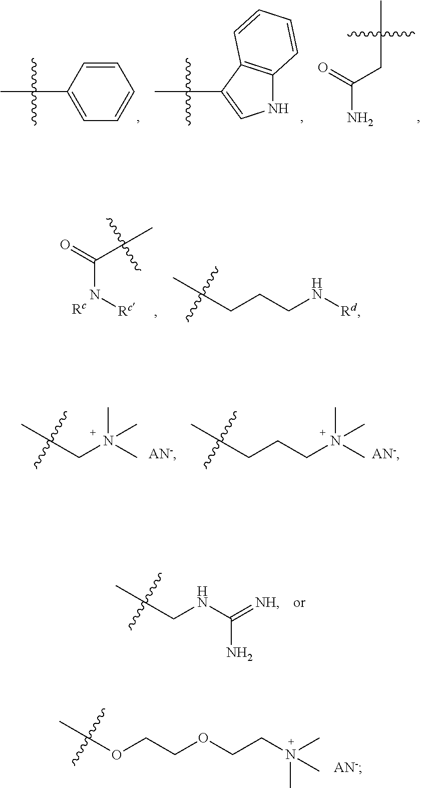

wherein:
   R^{c} is H, or —CH₃;
   R^{c}′ is H, or —CH₃;
   R^{d} is

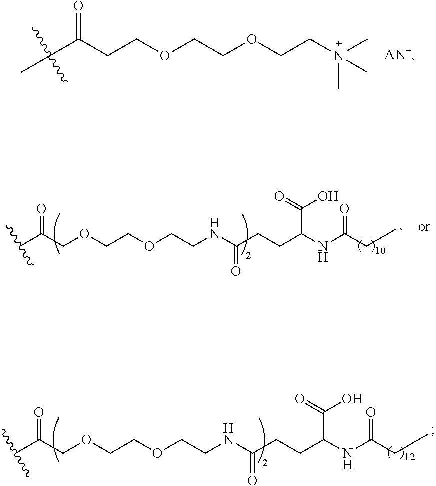

R³ is H, —CH₃, —CF₃, or

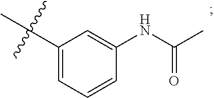 [SMILES: *c1cccc(NC(C)=O)c1]

R⁴ is H, or —CH₃;
X8 is

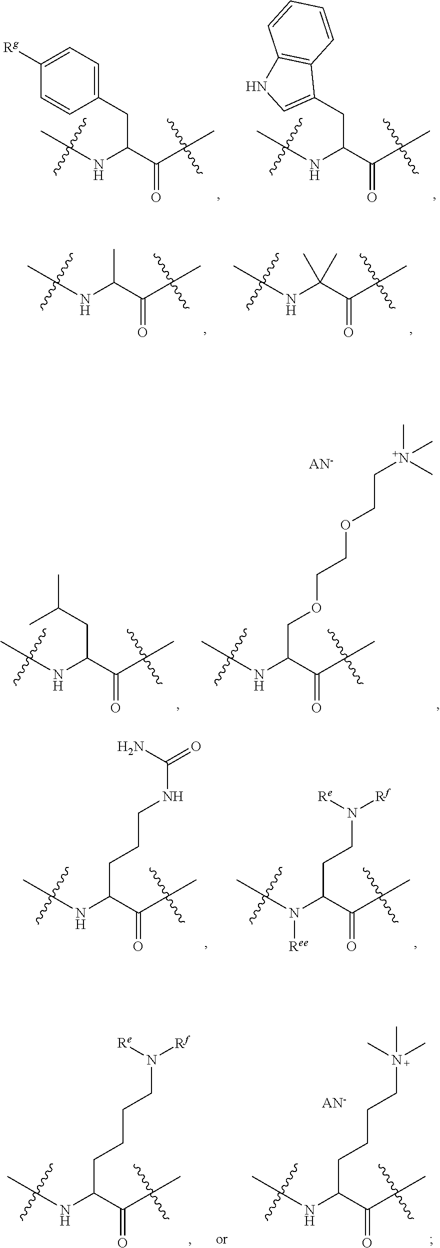

wherein:
   R^{e} is H, or —CH₃;
   R^{ee} is H;
   R^{f} is H, —CH₃, —C(CH₃)₃, —C(O)CH₃, —C(O)(CH₂CH₂O)₆CH₃, —(CH₂CH₂O)₆CH₃,

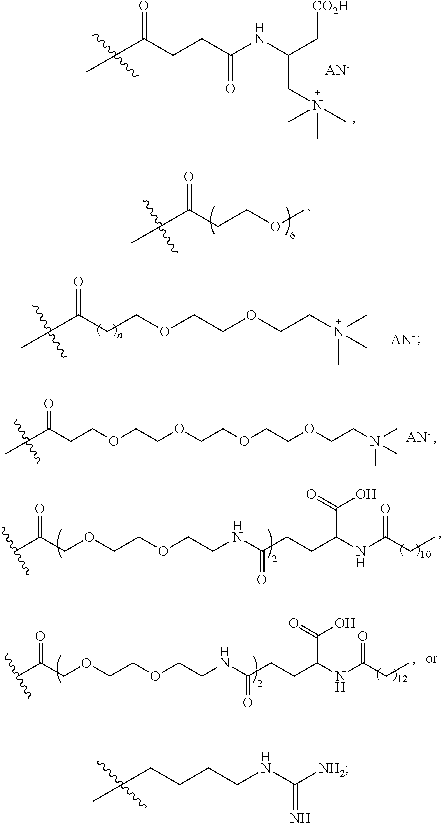

R^{g} is H, —OH, —C(O)NH₂, or —CH₂NHC(O)CH₃;
n is 0, or 1;
X12 is absent,

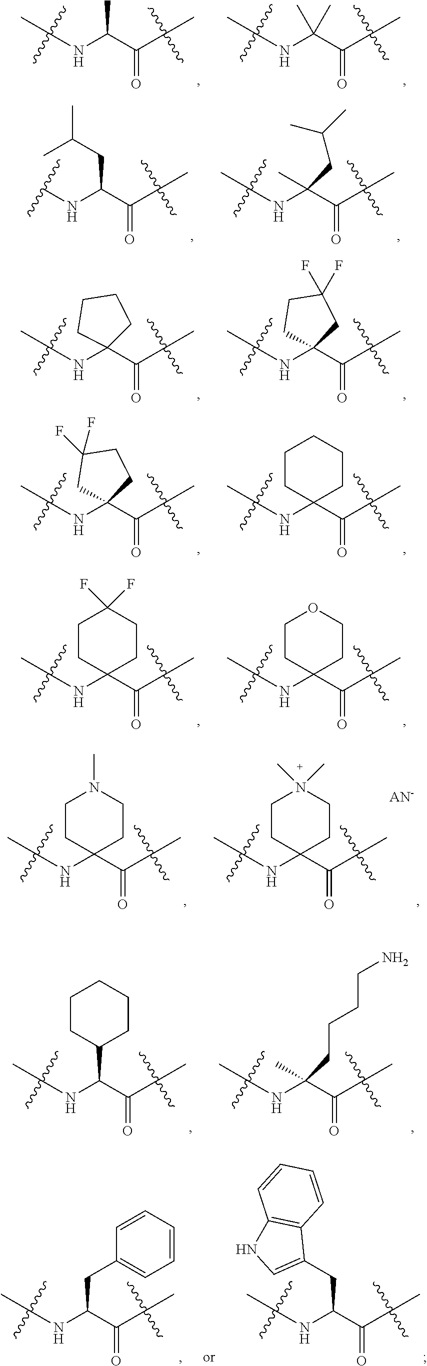

R⁶ is —CH₃—CH₂CH₂C(O)R^{m}, —(CH₂)ₛNRⁿR^{o},

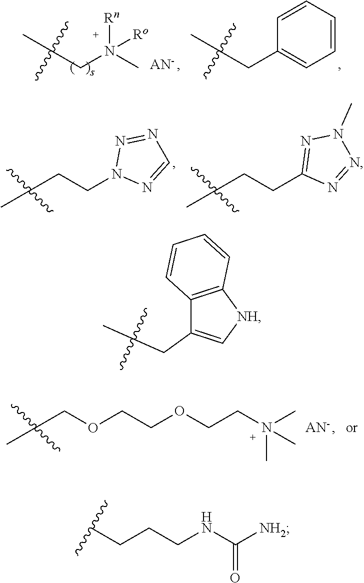

R⁷ is H, or —CH₃;
wherein:
   R^{m} is —OH, or

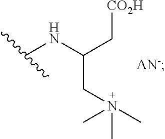

Rⁿ is H, —CH₃, —CH₂CH(CH₃)₂, —C(O)CH₃, —C(O)(CH₂CH₂O)₆CH₃, —(CH₂CH₂O)₆CH₃, —C(O)(CH₂CH₂O)ₜCH₂CH₂R^{p},

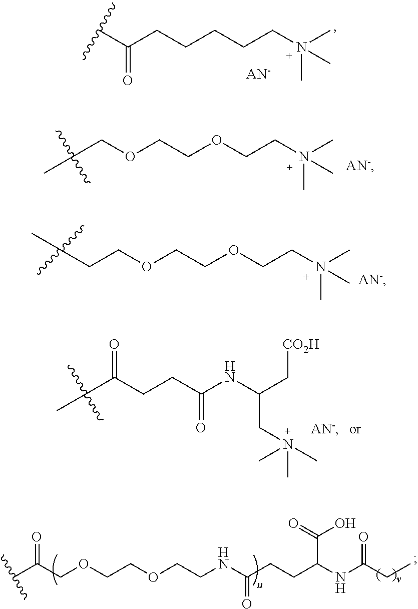

R^{o} is H, —CH₃, or —C(O)CH₃;
wherein:
   R^{p} is NHC(O)CH₃, or

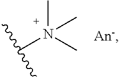

s is 1,2, or 4;
t is 2, 6, 9, or 12;
u is 2 or 3;
v is 10 or 12;
R⁸ is —CH₂CH₂CH₂NHC(O)CH₃, —C(O)NH₂, or —C(O)NHCH₃;
X15 is absent,

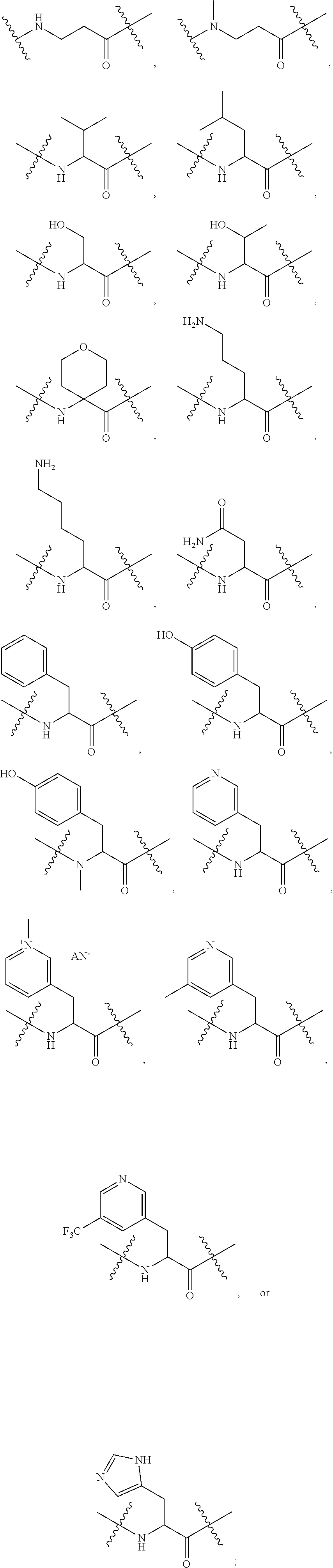

X16 is absent,

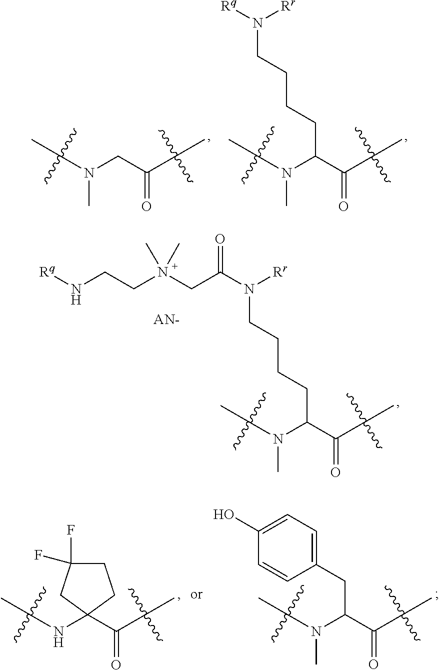

wherein:
   R^{q} is

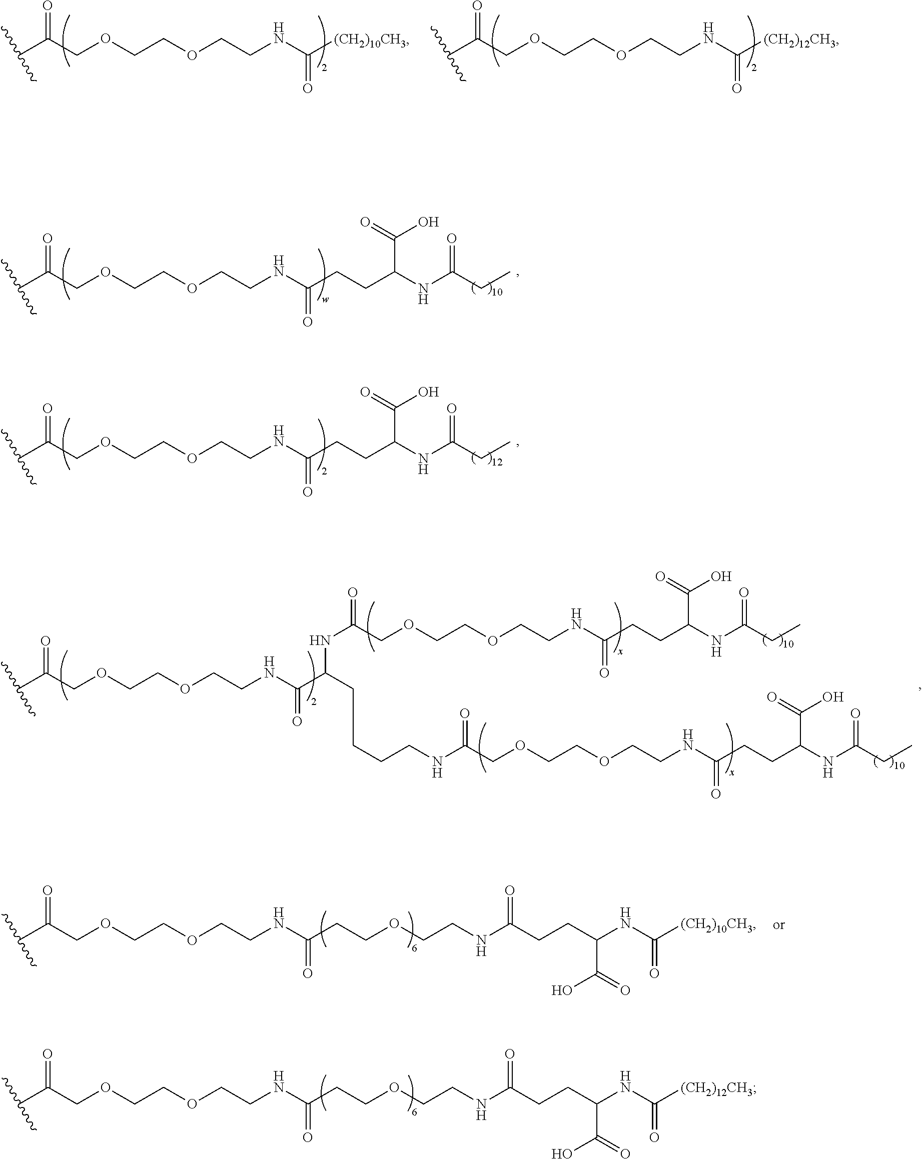

R^{r} is H;
w is 2 4, or 5;
x is 2 or 4; and
R⁹ is —NH₂, —NHCH₃, —N(CH₃)₂, —NH(CH₂CH₂O)₂CH₃, —N(CH₃)(CH₂CH₂O)₂CH₃, —NH(CH₂CH₂O)_{y}R^{s} or

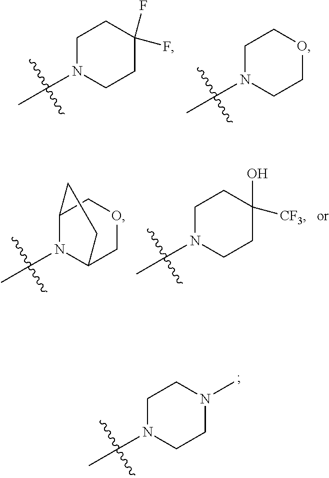

wherein:
   y is 2, 4, or 6;
   R^{s} is —CH₃, or

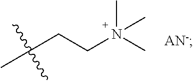

wherein:
   AN⁻as above defined for A, R, X3, X12, R1, R2, R5, R6, R8, R15, R16 and R^{a}, R^{b}, R^{d}, R^{f}, R^{h}, Rⁱ, R^{j}, R^{k}, R^{m}, Rⁿ, R^{o}, R^{s} is acetate, adipate, benzoate, benzenesulfonate, citrate, decanoate, chloride, lactate, maleate, methanesulfonate, oxalate, pivalate, propionate, succinate, sulfate, tartrate, or trifluoroacetate; or
a pharmaceutically acceptable salt thereof.

### Clause 17

17. The compound of clause 16 wherein
AN⁻is chloride;
or a pharmaceutically acceptable salt thereof.

### Clause 18

18. The compound of clause 16 which is
cPEG3aCO-Pen(3)—N(N(Me)2)-T-7MeW—K(NMeAC)-Pen(3)-TMAPF-2Nal-THP-K(NMeAC)—N-3Pya-NMeK(PEG2PEG2C12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG2C14)—CONH2;
C12gEPEG2PEG2CO-Pen(3)—N(N(Me)2)-T-7MeW—K(Ac)-Pen(3)-TMAPF-2Nal-THP-K(NMeAc)—N-3Pya-NMeK(PEG2PEG2gEC12)—CONH2;
cPEG3aCO-Pen(3)—K(PEG2PEG2gEC12)-T-7MeW—K(Ac)-Pen(3)-TMAPF-2Nal-THP-K(NMeAc)—N-3Pya-NMeK(PEG2PEG2gEC12)—CONH2;
cPEG3aCO-Pen(3)—N-T-7MeW—K(Ac)-Pen(3)-TMAPF-2Nal-THP-K(PEG2PEG2gEC12)—N-3Pya-NMeK(PEG2PEG2gEC12)—CONH2;
cPEG3aCO-Pen(3)—N-T-7MeW—K(Ac)-Pen(3)-TMAPF-2Nal-THP-K(NMeAc)—N-3Pya-NMeK(PEG2PEG2gEC12)—CONH2;
cPEG3aCO-Pen(3)—N(N(Me)2)-T-7MeW—K(Ac)-Pen(3)-TMAPF-2Nal-THP-K(NMeAC)—N-3Pya-NMeK(PEG2PEG2gEC12)—CONH2;
cPEG3aCO-Pen(3)—N(N(Me)2)-T-7MeW—K(NMeAC)-Pen(3)-TMAPF-2Nal-THP-K(NMeAC)—N-3Pya-NMeK(PEG2PEG2gEC12)—CONH2;
cPEG3aCO-k(PEG2PEG2gEC12)-Pen(3)—N(N(Me)2)-T-7MeW—K(Ac)-Pen(3)-TMAPF-2Nal-THP-K(NMeAc)—N-3Pya-NMeK(PEG2PEG2gEC12)—CONH2;
cPEG3aCO-Pen(3)—N(N(Me)2)-T-7MeW—K(PEG2PEG2gEC12)-Pen(3)-TMAPF-2Nal-THP-K(NMeAc)—N-3Pya-NMeK(PEG2PEG2gEC12)—CONH2;
cPEG3aCO-Pen(3)—N(N(Me)2)-T-7MeW—K(Ac)-Pen(3)-TMAPF-2Nal-THP-K(PEG2PEG2gEC12)—N-3Pya-NMeK(PEG2PEG2gEC12)—CONH2;
C14gEPEG2PEG2CO-Pen(3)—N(N(Me)2)-T-7MeW—K(Ac)-Pen(3)-TMAPF-2Nal-THP-K(NMeAc)—N-3Pya-NMeK(PEG2PEG2gEC14)—CONH2;
cPEG3aCO-Pen(3)—K(PEG2PEG2gEC14)-T-7MeW—K(Ac)-Pen(3)-TMAPF-2Nal-THP-K(NMeAc)—N-3Pya-NMeK(PEG2PEG2gEC14)—CONH2;
cPEG3aCO-Pen(3)—N(N(Me)2)-T-7MeW—K(NMeAC)-Pen(3)-TMAPF-2Nal-THP-K(NMeAC)—N-3Pya-NMeK(PEG2PEG2gEC14)—CONH2;
cPEG3aCO-k(PEG2PEG2gEC14)-Pen(3)—N(N(Me)2)-T-7MeW—K(Ac)-Pen(3)-TMAPF-2Nal-THP-K(NMeAc)—N-3Pya-NMeK(PEG2PEG2gEC14)—CONH2;
cPEG3aCO-Pen(3)—N(N(Me)2)-T-7MeW—K(Ac)-Pen(3)-TMAPF-2Nal-THP-K(PEG2PEG2gEC14)—N-3Pya-NMeK(PEG2PEG2gEC14)—CONH2;
cPEG3aCO-Pen(3)—N(N(Me)2)-T-7MeW—K(PEG2PEG2gEC14)-Pen(3)-TMAPF-2Nal-THP-K(NMeAc)—N-3Pya-NMeK(PEG2PEG2gEC14)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG2K(PEG2PEG2gEC12)₂)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG2K(PEG2PEG2PEG2PEG2gEC12)₂)—CONH2;
cPEG3aCO-Pen(3)—N(N(Me)2)-T-7MeW—K(Ac)-Pen(3)-TMAPF-2Nal-THP-K(PEG2PEG2PEG2gEC12))—N-3Pya-NMeK(PEG2PEG2PEG2gEC12)—CONH2;
cPEG3aCO-k(PEG2PEG2PEG2PEG2gEC12)-Pen(3)—N(N(Me)2)-T-7MeW—K(Ac)-Pen(3)-TMAPF-2Nal-THP-K(NMeAc)—N-3Pya-NMeK(PEG2PEG2PEG2PEG2gEC12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG6gEC12)—CONH2;
cPEG3aCO-k(PEG2PEG6gEC12)-Pen(3)—N(N(Me)2)-T-7MeW—K(Ac)-Pen(3)-TMAPF-2Nal-THP-K(NMeAc)—N-3Pya-NMeK(PEG2PEG6gEC12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(PEG2PEG6gEC14)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(SP6PEG2PEG2C12)—CONH2;
cPEG3aCO-Pen(3)—N(N(Me)2)-T-7MeW—K(Ac)-Pen(3)-TMAPF-2Nal-THP-K(NMeAc)—N-3Pya-NMeK(SP6PEG2PEG2gEC12)—CONH2;
4cpgCO-Pen(3)—N(N(Me)2)-T-7MeW—K(NMeAc)-Pen(3)-TMAPF-2Nal-THP-K(NMeAc)—N-3Pya-Sar-CON(Me)2;
5cpaCO-Pen(3)—N(N(Me)2)-T-7MeW-Q(N(Me)2)-Pen(3)-TMAPF-2Nal-THP-Q(N(Me)2)—N-3Pya-Sar-CON(Me)2;
5cpaCO-Pen(3)—N(N(Me)2)-T-7MeW—K(NMeAc)-Pen(3)-TMAPF-2Nal-THP-K(NMeAc)—N-3Pya-Sar-CON(Me)2;
C12gEPEG2PEG2CO-hk(Me)₃-Pen(3)—N(N(Me)2)-T-7MeW—K(NMeAc)-Pen(3)-TMAPF-2Nal-THP-K(NMeAc)—N-3Pya-Sar-CON(Me)2;
C12gEPEG2PEG2CO-hk(Me)₃-Pen(3)—N(N(Me)2)-T-7MeW—K(PEG2PEG2gEC12)-Pen(3)-TMAPF-2Nal-THP-K(NMeAc)—N-3Pya-Sar-CON(Me)2;
C12gEPEG2PEG2CO-hk(Me)₃-Pen(3)—N(N(Me)2)-T-7MeW—K(NMeAc)-Pen(3)-TMAPF-2Nal-THP-K(PEG2PEG2gEC12)—N-3Pya-Sar-CON(Me)2;
C12gEPEG2PEG2CO-hk(Me)₃-Pen(3)—N(N(Me)2)-T-7MeW—K(NMeAc)-Pen(3)-TMAPF-2Nal-K(PEG2PEG2gEC12)—N-3Pya-Sar-CON(Me)2;
C12gEPEG2PEG2CO-k(PEG2PEG2gEC12)-Pen(3)—N(N(Me)2)-T-7MeW-Dab(NMeAc)-Pen(3)-TMAPF-2Nal-THP-K(NMeAc)—N-3Pya-Sar-CON(Me)2;
C14gEPEG2PEG2CO-hk(Me)₃-Pen(3)—K(PEG2PEG2gEC14)-T-7MeW—K(NMeAc)-Pen(3)-TMAPF-2Nal-THP-K(NMeAc)—N-3Pya-Sar-CON(Me)2;
C14gEPEG2PEG2CO-hk(Me)₃-Pen(3)—N(N(Me)2)-T-7MeW—K(NMeAc)-Pen(3)-TMAPF-2Nal-THP-K(NMeAc)—N-3Pya-Sar-CON(Me)2;
C14gEPEG2PEG2CO-hk(Me)₃-Pen(3)—N(N(Me)2)-T-7MeW—K(PEG2PEG2gEC14)-Pen(3)-TMAPF-2Nal-THP-K(NMeAc)—N-3Pya-Sar-CON(Me)2;
C14gEPEG2PEG2CO-hk(Me)₃-Pen(3)—N(N(Me)2)-T-7MeW—K(NMeAc)-Pen(3)-TMAPF-2Nal-THP-K(PEG2PEG2gEC14)—N-3Pya-Sar-CON(Me)2;
C14gEPEG2PEG2CO-k(PEG2PEG2gEC14)-Pen(3)—N(N(Me)2)-T-7MeW-Dab(NMeAc)-Pen(3)-TMAPF-2Nal-THP-K(NMeAc)—N-3Pya-Sar-CON(Me)2;
cPEG2gCO-Pen(3)—N(N(Me)2)-T-7MeW—K(NMeAc)-Pen(3)-TMAPF-2Nal-THP-K(NMeAc)—N-3Pya-Sar-CON(Me)2;
cPEG3aCO-Pen(3)—K(cPEG3a)-T-7MeW—K(NMeAc)-Pen(3)-TMAPF-2Nal-THP-K(NMeAc)—N-3Pya-Sar-CON(Me)2;
cPEG3aCO-Pen(3)—K(Me)₃-T-7MeW—K(NMeAc)-Pen(3)-TMAPF-2Nal-THP-K(NMeAc)—N-3Pya-Sar-CON(Me)2;
cPEG3aCO-Pen(3)—K(Me)₃-T-7MeW—K(NMeAc)-Pen(3)-TMAPF-2Nal-Achx-K(NMeAc)—N-3Pya-Sar-CON(Me)2;
cPEG3aCO-k(PEG2PEG2gEC12)-Pen(3)—K(PEG2PEG2gEC12)-T-7MeW—K(NMeAc)-Pen(3)-TMAPF-2Nal-THP-K(NMeAc)—N-3Pya-Sar-CON(Me)2;
cPEG3aCO-Pen(3)—K(PEG2PEG2gEC12)-T-7MeW—K(NMeAc)-Pen(3)-TMAPF-2Nal-THP-K(NMeAc)—N-3Pya-Sar-CON(Me)2;
cPEG3aCO-Pen(3)—K(PEG2PEG2gEC12)-T-7MeW—K(PEG2PEG2gEC12)-Pen(3)-TMAPF-2Nal-THP-K(NMeAc)—N-3Pya-Sar-CON(Me)2;
cPEG3aCO-Pen(3)—K(PEG2PEG2gEC12)-T-7MeW—K(NMeAc)-Pen(3)-TMAPF-2Nal-THP-K(PEG2PEG2gEC12)—N-3Pya-Sar-CON(Me)2;
cPEG3aCO-k(PEG2PEG2gEC14)-Pen(3)—K(PEG2PEG2gEC14)-T-7MeW—K(NMeAc)-Pen(3)-TMAPF-2Nal-THP-K(NMeAc)—N-3Pya-Sar-CON(Me)2;
cPEG3aCO-Pen(3)—K(PEG2PEG2gEC14)-T-7MeW—K(NMeAc)-Pen(3)-TMAPF-2Nal-THP-K(NMeAc)—N-3Pya-Sar-CON(Me)2;
cPEG3aCO-Pen(3)—K(PEG2PEG2gEC14)-T-7MeW—K(PEG2PEG2gEC14)-Pen(3)-TMAPF-2Nal-THP-K(NMeAc)—N-3Pya-Sar-CON(Me)2;
cPEG3aCO-Pen(3)—K(PEG2PEG2gEC14)-T-7MeW—K(NMeAc)-Pen(3)-TMAPF-2Nal-THP-K(PEG2PEG2gEC14)—N-3Pya-Sar-CON(Me)2;
cPEG3aCO-Pen(3)—N-T-7MeW—K(NMeAc)-Pen(3)-TMAPF-2Nal-THP-K(NMeAc)—N-3Pya-Sar-CONH2;
cPEG3aCO-Pen(3)—N-T-7MeW—K(NMeAc)-Pen(3)-TMAPF-2Nal-THP-K(NMeAc)—N-3Pya-Sar-CON(Me)2;
cPEG3aCO-Pen(3)—N-T-7MeW—K(NMeAC)-Pen(3)-TMAPF-2Nal-THP-Dab(NMeAc)—N-3Pya-Sar-CON(Me)2;
cPEG3aCO-Pen(3)—N-T-7MeW-Dab(NMeAc)-Pen(3)-TMAPF-2Nal-THP-K(NMeAC)—N-3Pya-Sar-CON(Me)2;
cPEG3aCO-Pen(3)—N-T-7MeW—K(Ac)-Pen(3)-TMAPF-2Nal-THP-A-N-3Pya-Sar-CON(Me)2;
cPEG3aCO-Pen(3)—N-T-7MeW—K(Ac)-Pen(3)-TMAPF-2Nal-THP-E-N-3Pya-Sar-CONH2;
cPEG3aCO-Pen(3)—N-T-7MeW—K(Ac)-Pen(3)-TMAPF-2Nal-THP—K(Ac)—N-3Pya-Sar-CONH2;
cPEG3aCO-Pen(3)—N-T-7(3NAcPh)W—K(Ac)-Pen(3)-TMAPF-2Nal-THP-E-N-3Pya-Sar-CONH2;
cPEG3aCO-Pen(3)—N(N(Me))-T-7MeW—K(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-THP-E-N-3Pya-Sar-CON(Me)2;
cPEG3aCO-Pen(3)—N(N(Me)2)-T-7MeW—K(NMeAc)-Pen(3)-TMAPF-2Nal-THP-K(NMeAc)—N-3Pya-Sar-CO(NHPEG3a);
cPEG3aCO-Pen(3)—N(N(Me)2)-T-7MeW-APEG2Ser(S*)-Pen(3)-TMAPF-2Nal-THP-K(NMeAc)—N-3Pya-Sar-CON(Me)2;
cPEG3aCO-Pen(3)—N(N(Me)2)-T-7MeW-APEG2Ser(R*)-Pen(3)-TMAPF-2Nal-THP-K(NMeAc)—N-3Pya-Sar-CON(Me)2;
cPEG3aCO-Pen(3)—N(N(Me)2)-T-7(3NAcPh)W—K(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(NMeAC)-Pen(3)-TMAPF-2Nal-THP-Dab(NMeAc)—N-3Pya-Sar-CON(Me)2;
cPEG3aCO-Pen(3)—N(N(Me)2)-T-7MeW—K(Ac)-Pen(3)-TMAPF-2Nal-THP-Dab(NMeAc)—N-3Pya-Sar-CON(Me)2;
cPEG3aCO-Pen(3)—N(N(Me)2)-T-7MeW—K(NMeAC)-Pen(3)-TMAPF-2Nal-THP-Q(N(Me)2)—N-3Pya-Sar-CON(Me)2;
cPEG3aCO-Pen(3)—N(N(Me)2)-T-7MeW-Q(N(Me)2)-Pen(3)-TMAPF-2Nal-THP-K(NMeAC)—N-3Pya-Sar-CON(Me)2;
cPEG3aCO-Pen(3)—N(N(Me)2)-T-7MeW—K(NMeAc)-Pen(3)-TMAPF-2Nal-THP-A-N-3Pya-Sar-CON(Me)2;
cPEG3aCO-Pen(3)—N(N(Me)2)-T-7MeW—K(NMeAc)-Pen(3)-TMAPF-2Nal-THP-K(NMeAc)—N-3Pya-Sar-CONH2;
cPEG3aCO-Pen(3)—N(N(Me)2)-T-7MeW—K(NMeAc)-Pen(3)-TMAPF-2Nal-THP-Cit-N-3Pya-Sar-CON(Me)2;
cPEG3aCO-Pen(3)—N(N(Me)2)-T-7MeW—K(Me)₃-Pen(3)-TMAPF-2Nal-THP-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-Acpx-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-Achx-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-Chg-K(NMeAc)—N-3Pya-Sar-CON(Me)2;
cPEG3aCO-Pen(3)—N(N(Me)2)-T-7MeW—K(NMeAc)-Pen(3)-TMAPF-2Nal-Aib-K(NMeAc)—N-3Pya-Sar-CON(Me)2;
cPEG3aCO-Pen(3)—N(N(Me)2)-T-7MeW-Cit-Pen(3)-TMAPF-2Nal-THP-K(NMeAc)—N-3Pya-Sar-CON(Me)2;
cPEG3aCO-Pen(3)—N(N(Me)2)-T-7MeW-Dab(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW-Dab(NMeAc)-Pen(3)-TMAPF-2Nal-THP-K(NMeAc)—N-3Pya-Sar-CONH2;
cPEG3aCO-k(PEG2PEG2gEC12)-Pen(3)—N(N(Me)2)-T-7MeW—K(NMeAc)-Pen(3)-TMAPF-2Nal-THP-K(NMeAc)—N-3Pya-Sar-CON(Me)2;
cPEG3aCO-k(PEG2PEG2gEC14)-Pen(3)—N(N(Me)2)-T-7MeW—K(NMeAc)-Pen(3)-TMAPF-2Nal-THP-K(NMeAc)—N-3Pya-Sar-CON(Me)2;
cPEG3aCO-k(PEG2PEG2gEC12)-Pen(3)—N(N(Me)2)-T-7MeW—K(PEG2PEG2gEC12)-Pen(3)-TMAPF-2Nal-THP-K(NMeAc)—N-3Pya-Sar-CON(Me)2;
cPEG3aCO-Pen(3)—N(N(Me)2)-T-7MeW—K(cPEG3a)-Pen(3)-TMAPF-2Nal-THP-K(NMeAc)—N-3Pya-Sar-CON(Me)2;
cPEG3aCO-Pen(3)—N(N(Me)2)-T-7MeW—K(4cpg)-Pen(3)-TMAPF-2Nal-THP-K(NMeAc)—N-3Pya-Sar-CON(Me)2;
cPEG3aCO-k(PEG2PEG2gEC12)-Pen(3)—N(N(Me)2)-T-7MeW—K(NMeAc)-Pen(3)-TMAPF-2Nal-THP-K(PEG2PEG2gEC12)—N-3Pya-Sar-CON(Me)2;
cPEG3aCO-k(PEG2PEG2gEC14)-Pen(3)—N(N(Me)2)-T-7MeW—K(NMeAc)-Pen(3)-TMAPF-2Nal-THP-K(PEG2PEG2gEC14)—N-3Pya-Sar-CON(Me)2;
cPEG3aCO-Pen(3)—N(N(Me)2)-T-7MeW—K(PEG2PEG2gEC14)-Pen(3)-TMAPF-2Nal-THP-K(NMeAc)—N-3Pya-Sar-CON(Me)2;
cPEG3aCO-Pen(3)—N(N(Me)2)-T-7MeW—K(PEG2PEG2gEC12)-Pen(3)-TMAPF-2Nal-THP-K(PEG2PEG2gEC12)—N-3Pya-Sar-CON(Me)2;
cPEG3aCO-Pen(3)—N(N(Me)2)-T-7MeW—K(NMeAc)-Pen(3)-TMAPF-2Nal-THP-K(PEG2PEG2gEC12)—N-3Pya-Sar-CON(Me)2;
cPEG3aCO-Pen(3)—N(N(Me)2)-T-7MeW—K(NMeAc)-Pen(3)-TMAPF-2Nal-THP-K(PEG2PEG2gEC14)—N-3Pya-Sar-CON(Me)2;
cPEG3aCO-Pen(3)—N(N(Me)2)-T-7MeW—K(PEG2PEG2gEC14)-Pen(3)-TMAPF-2Nal-THP-K(PEG2PEG2gEC14)—N-3Pya-Sar-CON(Me)2;
cPEG3aCO-Pen(3)—N(N(Me)2)-T-7MeW-Dap(NMeAc)-Pen(3)-TMAPF-2Nal-THP-K(NMeAc)—N-3Pya-Sar-CON(Me)2;
cPEG3aCO-Pen(3)—N(N(Me)2)-T-7MeW—K(Ac)-Pen(3)-TMAPF-2Nal-THP-Dap(NMeAc)—N-3Pya-Sar-CON(Me)2;
cPEG3gCO-Pen(3)—N(N(Me)2)-T-7MeW—K(NMeAc)-Pen(3)-TMAPF-2Nal-THP-K(NMeAc)—N-3Pya-Sar-CON(Me)2;
MeCO-Pen(3)—N-T-7MeW—K(NMeAc)-Pen(3)-TMAPF-2Nal-THP-K(NMeAc)—N-3Pya-Sar-CONH2;
MeCO-Pen(3)—N-T-7MeW—K(Ac)-Pen(3)-TMAPF-2Nal-THP—K(Ac)—N-3Pya-Sar-CONH2;
MeCO-Pen(3)—N-T-7MeW—K(Ac)-Pen(3)-TMAPF-2Nal-THP-E-N-3Pya-Sar-CONH2;
MeCO-k(Me)₃-Pen(3)—N-T-7MeW—K(Ac)-Pen(3)-TMAPF-2Nal-THP-E-N-3Pya-Sar-CONH2;
MeCO-k(Me)₃-Pen(3)—N-T-7(3NAcPh)W—K(Ac)-Pen(3)-TMAPF-2Nal-THP-E-N-3Pya-Sar-CONH2;
MeCO-k(Me)₃-Pen(3)—N-T-7MeW—K(Ac)-Pen(3)-TMAPF-2Nal-THP—K(Ac)—N-3Pya-Sar-CONH2;
MeCO-Pen(3)—N-T-7(3NAcPh)W—K(Ac)-Pen(3)-TMAPF-2Nal-THP-E-N-3Pya-Sar-CONH2;
MeCO-Pen(3)—N-T-7(3NAcPh)W—K(Ac)-Pen(3)-TMAPF-2Nal-THP—K(Ac)—N-3Pya-Sar-CONH2;
MeCO-r-Pen(3)—N-T-7MeW—K(Ac)-Pen(3)-TMAPF-2Nal-THP-E-N-3Pya-Sar-CONH2;
MeCO-Pen(3)—N(N(Me)2)-T-7MeW—K(NMeAc)-Pen(3)-TMAPF-2Nal-THP-K(NMeAc)—N-3Pya-Sar-CON(Me)2;
MeCO-Pen(3)—N(N(Me)2)-T-7MeW—K(NMeAc)-Pen(3)-TMAPF-2Nal-THIP-K(NMeAc)—N-3Pya-Sar-CO(NHPEG3a);
MeCO-Pen(3)—N(N(Me)2)-T-7MeW—K(NMeAc)-Pen(3)-TMAPF-2Nal-THP-APEG2Ser-N-3Pya-Sar-CON(Me)2;
MeCO-hk(Me)₃-Pen(3)—N(N(Me)2)-T-7MeW—K(NMeAc)-Pen(3)-TMAPF-2Nal-THP-K(NMeAc)—N-3Pya-Sar-CON(Me)2;
MeCO-r-Pen(3)—N(N(Me)2)-T-7MeW—K(NMeAc)-Pen(3)-TMAPF-2Nal-THP-K(NMeAc)—N-3Pya-Sar-CON(Me)2; or
   a pharmaceutically acceptable salt thereof.

### Clause 19

19. A compound which is:

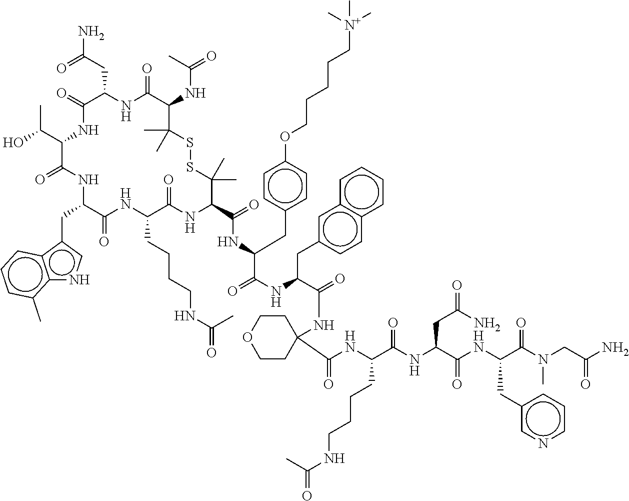

or a pharmaceutically acceptable salt thereof.

### Clause 20

20. A compound which is

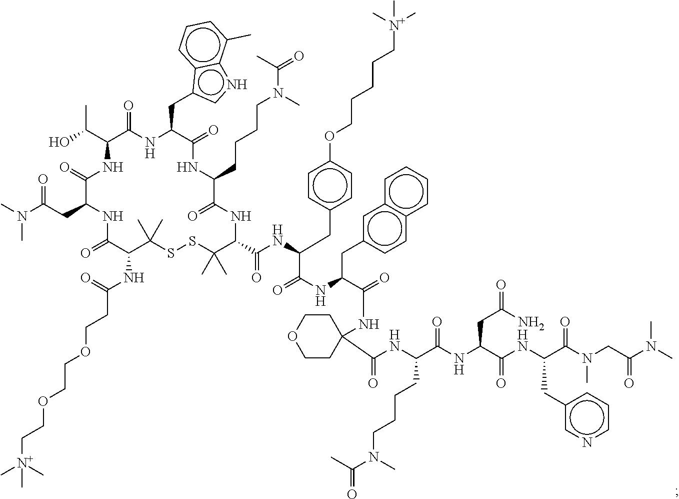

pharmaceutically acceptable salt thereof.

### Clause 21

21. The compound or pharmaceutically acceptable salt thereof, according to any one of clauses 19 and 20 wherein the quarternary ammonium salts as shown in the chemical structures therein include a counterion AN⁻selected from: acetate, adipate, benzoate, benzenesulfonate, citrate, decanoate, chloride, lactate, maleate, methanesulfonate, oxalate, pivalate, propionate, succinate, sulfate, tartrate, or trifluoroacetate.

Clause 22. A pharmaceutical composition comprising the peptide inhibitor or pharmaceutically acceptable salt thereof of any one of clauses 1-21, and a pharmaceutically acceptable carrier, excipient, or diluent.
Clause 23. The pharmaceutical composition of clause 22, further comprising an enteric coating.
Clause 24. The pharmaceutical composition of clause 22, wherein the enteric coating protects and releases the pharmaceutical composition within a subject's lower gastrointestinal system.

### Clause 25.

25. A method for treating an Inflammatory Bowel Disease (IBD), ulcerative colitis, Crohn's disease, Celiac disease (nontropicalSprue), enteropathy associated with seronegative arthropathies, microscopic colitis, collagenous colitis, eosinophilic gastroenteritis, colitis associated with radio- or chemo-therapy, colitis associated with disorders of innate immunity as in leukocyte adhesion deficiency-1, chronic granulomatous disease, glycogen storage disease type 1b, Hermansky-Pudlak syndrome, Chediak-Higashi syndrome, and Wiskott-Aldrich Syndrome, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, psoriasis, psoriatic arthritis, or graft versus host disease in a subject, comprising providing to the subject an effective amount of the peptide inhibitor or peptide inhibitor or pharmaceutically acceptable salt thereof of any one of clauses 1-21, or the pharmaceutical composition of any one of clauses 22-24.

### Clause 26

26. The method of clause 25, wherein the pharmaceutical composition is provided to the subject by an oral, parenteral, intravenous, peritoneal, intradermal, subcutaneous, intramuscular, intrathecal, inhalation, vaporization, nebulization, sublingual, buccal, parenteral, rectal, intraocular, inhalation, topically, vaginal, or topical route of administration.

### Clause 27

27. The method of clause 24 for treating Inflammatory Bowel Disease (IBD), ulcerative colitis, or Crohn's disease, wherein the pharmaceutical composition is provided to the subject orally.

### Clause 28

28. The method of clause 24 for treating psoriasis, wherein the pharmaceutical composition is provided to the subject orally, topically, parenterally, intravenously, subcutaneously, peritonealy, or intravenously.

### Clause 29

29. The peptide inhibitor or peptide inhibitor or pharmaceutically acceptable salt thereof of any one of clauses 1-21, or the pharmaceutical composition of any one of clauses 22-24, for use in the treatment of an Inflammatory Bowel Disease (IBD), ulcerative colitis, Crohn's disease, Celiac disease (nontropicalSprue), enteropathy associated with seronegative arthropathies, microscopic colitis, collagenous colitis, eosinophilic gastroenteritis, colitis associated with radio- or chemo-therapy, colitis associated with disorders of innate immunity as in leukocyte adhesion deficiency-1, chronic granulomatous disease, glycogen storage disease type 1b, Hermansky-Pudlak syndrome, Chediak-Higashi syndrome, and Wiskott-Aldrich Syndrome, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, psoriasis, psoriatic arthritis, or graft versus host disease in a subject, comprising providing to the subject an effective amount of the peptide inhibitor or peptide inhibitor or pharmaceutically acceptable salt thereof of any one of clauses 1-21, or the pharmaceutical composition of any one of clauses 22-24.

### Clause 30

30. The composition for use according to clause 29, wherein the pharmaceutical composition is provided to the subject by an oral, parenteral, intravenous, peritoneal, intradermal, subcutaneous, intramuscular, intrathecal, inhalation, vaporization, nebulization, sublingual, buccal, parenteral, rectal, intraocular, inhalation, topically, vaginal, or topical route of administration.

### Clause 31

31. The composition for use according to clause 29 for treating Inflammatory Bowel Disease (IBD), ulcerative colitis, or Crohn's disease, wherein the pharmaceutical composition is provided to the subject orally.

### Clause 32

32. The composition for use according to clause 29 for treating psoriasis, wherein the pharmaceutical composition is provided to the subject orally, topically, parenterally, intravenously, subcutaneously, peritonealy, or intravenously.

### Enumerated Embodiments

The present disclosure further includes the following enumerated embodiments.

### Numbered Embodiment 1

1. A cyclic peptide, or a pharmaceutically acceptable salt or solvate thereof, comprising an amino acid sequence of Formula (A):

Z³-Z⁴-Z⁵-Z⁶-Z⁷-Z⁸-Z⁹-Z¹⁰-Z¹¹-Z¹²-Z¹³-Z¹⁴-
Z¹⁵-Z¹⁶ (A),

wherein:
the amino acid residue at position Z³ is absent or the residue of r;
the amino acid residue at position Z⁴ is the residue of an amino acid that is connected to the amino acid residue at position Z⁹;
the amino acid residue at position Z⁵ is the residue of N(N(Me)₂);
the amino acid residue at position Z⁶ is the residue of T;
the amino acid residue at position Z⁷ is the residue of 7MeW;
the amino acid residue at position Z⁸ is the residue of K(NMeAc);
the amino acid residue at position Z⁹ is the residue of an amino acid that is connected to the amino acid residue at position Z⁴;
the amino acid residue at position Z¹⁰ is the residue of TMAPF;
the amino acid residue at position Z¹¹ is the residue of 2Nal;
the amino acid residue at position Z¹² is absent or the residue of THP;
the amino acid residue at position Z¹³ is the residue of K(NMeAc);
the amino acid residue at position Z¹⁴ is the residue of N;
the amino acid residue at position Z¹⁵ is absent or the residue of 3Pya;
the amino acid residue at position Z¹⁶ is absent or the residue of Sar;
eight or fewer of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position; and
at least one of the amino acid residues at positions Z³, Z⁵, Z⁶, Z⁷, Z⁸, Z¹⁰, Z¹¹, Z¹², Z¹³, Z¹⁴, Z¹⁵ and/or Z¹⁶, when an amino acid residue is present at the position, each independently comprise(s) at least one quaternary amine.

### Numbered Embodiment 2

2. A cyclic peptide, or a pharmaceutically acceptable salt or solvate thereof, comprising an amino acid sequence of Formula (B):

Z³-Z⁴-Z⁵-Z⁶-Z⁷-Z⁸-Z⁹-Z¹⁰-Z¹¹-Z¹²-Z¹³-Z¹⁴-
Z¹⁵-Z¹⁶ (B),

wherein:
the amino acid residue at position Z³ is absent or the residue of r;
the amino acid residue at position Z⁴ is the residue of an amino acid that is connected to the amino acid residue at position Z⁹;
the amino acid residue at position Z⁵ is the residue of N(N(Me)₂);
the amino acid residue at position Z⁶ is the residue of T;
the amino acid residue at position Z⁷ is the residue of 7MeW;
the amino acid residue at position Z⁸ is the residue of K(NMeAc);
the amino acid residue at position Z⁹ is the residue of an amino acid that is connected to the amino acid residue at position Z⁴;
the amino acid residue at position Z¹⁰ is the residue of TMAPF;
the amino acid residue at position Z¹¹ is the residue of 2Nal;
the amino acid residue at position Z¹² is absent or the residue of THP;
the amino acid residue at position Z¹³ is the residue of K(NMeAc);
the amino acid residue at position Z¹⁴ is the residue of N;
the amino acid residue at position Z¹⁵ is absent or the residue of 3Pya;
the amino acid residue at position Z¹⁶ is absent or the residue of Sar;
eight or fewer of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position; and
at least one of the amino acid residues at positions Z³, Z⁵, Z⁶, Z⁷, Z⁸, Z¹⁰, Z¹¹, Z¹², Z¹³, Z¹⁴, Z¹⁵ and/or Z¹⁶, when an amino acid residue is present at the position, each independently comprise(s) at least one masked amine and/or masked amide.

### Numbered Embodiment 3

3. A cyclic peptide, or a pharmaceutically acceptable salt or solvate thereof, comprising an amino acid sequence of Formula (C):

Z³-Z⁴-Z⁵-Z⁶-Z⁷-Z⁸-Z⁹-Z¹⁰-Z¹¹-Z¹²-Z¹³-Z¹⁴-
Z¹⁵-Z¹⁶ (C),

wherein:
the amino acid residue at position Z³ is absent or the residue of r;
the amino acid residue at position Z⁴ is the residue of an amino acid that is connected to the amino acid residue at position Z⁹;
the amino acid residue at position Z⁵ is the residue of N(N(Me)₂);
the amino acid residue at position Z⁶ is the residue of T;
the amino acid residue at position Z⁷ is the residue of 7MeW;
the amino acid residue at position Z⁸ is the residue of K(NMeAc);
the amino acid residue at position Z⁹ is the residue of an amino acid that is connected to the amino acid residue at position Z⁴;
the amino acid residue at position Z¹⁰ is the residue of TMAPF;
the amino acid residue at position Z¹¹ is the residue of 2Nal;
the amino acid residue at position Z¹² is absent or the residue of THP;
the amino acid residue at position Z¹³ is the residue of K(NMeAc);
the amino acid residue at position Z¹⁴ is the residue of N;
the amino acid residue at position Z¹⁵ is absent or the residue of 3Pya;
the amino acid residue at position Z¹⁶ is absent or the residue of Sar; and
eight or fewer of the amino acid residues at positions Z³, Z⁵, Z⁶, Z⁷, Z⁸, Z°, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

### Numbered Embodiment 4

4. The cyclic peptide of any one of the preceding embodiments, wherein the amino acid residue at position Z⁴ is the residue of Abu or the residue of an amino acid comprising a sulfhydryl group.

### Numbered Embodiment 5

5. The cyclic peptide of any one of the preceding embodiments, wherein the amino acid residue at position Z⁹ is the residue of an amino acid comprising a sulfhydryl group.

### Numbered Embodiment 6

6. The cyclic peptide of any one of the preceding embodiments, wherein when the amino acid residue at position Z⁴ is the residue of an amino acid comprising a sulfhydryl group, the amino acid residue at position Z⁴ is connected to the amino acid residue at position Z⁹ by a disulfide bond formed between the amino acid comprising a sulfhydryl group at position Z⁴ and the amino acid comprising a sulfhydryl group at position Z⁹.

### Numbered Embodiment 7

7. The cyclic peptide of any one of the preceding embodiments, wherein the amino acid residue at position Z⁴ is the residue of an amino acid comprising a sulfhydryl group, and the amino acid residue at position Z⁴ is connected to the amino acid residue at position Z⁹ by a disulfide bond formed between the amino acid comprising a sulfhydryl group at position Z⁴ and the amino acid comprising a sulfhydryl group at position Z⁹.

### Numbered Embodiment 8

8. The cyclic peptide of any one of the preceding embodiments, wherein when the amino acid residue at position Z⁴ is the residue of Abu, the amino acid residue at position Z⁴ is connected to the amino acid residue at position Z⁹ by a thioether bond formed between the Abu at position Z⁴ and the amino acid comprising a sulfhydryl group at position Z⁹.

### Numbered Embodiment 9

9. The cyclic peptide of any one of the preceding embodiments, wherein the amino acid residue at position Z⁴ is the residue of Abu, and the amino acid residue at position Z⁴ is connected to the amino acid residue at position Z⁹ by a thioether bond formed between the Abu at position Z⁴ and the amino acid comprising a sulfhydryl group at position Z⁹.

### Numbered Embodiment 10

10. The cyclic peptide of any one of the preceding embodiments, wherein the cyclic peptide further comprises R^{NT}, wherein R^{NT} is bound to the N-terminal amine of the amino acid residue at position
(i) Z³ when Z³ is present, or
(ii) Z⁴ when Z³ is absent; and
   R^{NT} is selected from the group consisting of: —C(O)-optionally substituted (C₁-C₂₀) alkyl and —C(O)-optionally substituted (C₁-C₄₀) heteroalkyl.

### Numbered Embodiment 11

11. The cyclic peptide of any one of the preceding embodiments, wherein the cyclic peptide further comprises R^{CT}, wherein R^{CT} is bound to the carbonyl derived from the C-terminal carboxylic acid of the amino acid residue at position
(i) Z¹⁶ when Z¹⁶ is present,
(ii) Z¹⁵ when Z¹⁶ is absent, or
(iii) Z¹⁴ when Z¹¹ and Z¹⁶ are absent; and
   R^{CT} is —N(R^{Y})(R^{Z}), wherein
(i) each R^{Y} and R^{Z} is independently selected from the group consisting of: hydrogen, optionally substituted (C₁-C₁₅) alkyl and optionally substituted (C₁-C₃₀) heteroalkyl, or
(ii) each R^{Y} and R^{Z} come together with the N atom to which they are attached to form an optionally substituted (CH₃—C₁₄) heterocyclic ring or an optionally substituted (C₅-C₁₀) bicyclic heterocyclic ring.

### Numbered Embodiment 12

12. The cyclic peptide of any one of the preceding embodiments, wherein the cyclic peptide further comprises R^{CT}, wherein R^{CT} is bound to the carbonyl derived from the C-terminal carboxylic acid of the amino acid residue at position
(i) Z¹⁶ when Z¹⁶ is present,
(ii) Z¹⁵ when Z¹⁶ is absent, or
(iii) Z¹⁴ when Z¹⁵ and Z¹⁶ are absent; and
   R^{CT} is —N(R^{Y})(R^{Z}), wherein
(i) each R^{Y} and R^{Z} is independently selected from the group consisting of: hydrogen, optionally substituted (C₁-C₁₅) alkyl and optionally substituted (C₁-C₃₀) heteroalkyl, provided that only one of R and R^{Z} is hydrogen, or
(ii) each R^{Y} and R^{Z} come together with the N atom to which they are attached to form an optionally substituted (CH₃—C₁₄) heterocyclic ring or an optionally substituted (C₅-C₁₀) bicyclic heterocyclic ring.

### Numbered Embodiment 13

13. The cyclic peptide of any one of the preceding embodiments, wherein the cyclic peptide further comprises R^{NT} and R^{CT} and is of Formula (D):

R^{NT}-Z³-Z⁴-Z⁵-Z⁶-Z⁷-Z⁸-Z⁹-Z¹⁰-Z¹¹-Z¹²-
Z¹³-Z¹⁴-Z¹⁵-Z¹⁶-R^{CT} (D),

wherein R^{NT}, Z³, Z⁴, Z⁵, Z⁶, Z⁷, Z⁸, Z⁹, Z¹⁰, Z¹¹, Z¹², Z¹³, Z¹⁴, Z¹⁵, Z¹⁶ and R^{CT} are as defined in any one of the preceding embodiments.

### Numbered Embodiment 14

14. The cyclic peptide of any one of the preceding embodiments, wherein eight of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

### Numbered Embodiment 15

15. The cyclic peptide of any one of the preceding embodiments, wherein seven or fewer of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

### Numbered Embodiment 16

16. The cyclic peptide of any one of the preceding embodiments, wherein seven of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

### Numbered Embodiment 17

17. The cyclic peptide of any one of the preceding embodiments, wherein six or fewer of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

### Numbered Embodiment 18

18. The cyclic peptide of any one of the preceding embodiments, wherein six of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

### Numbered Embodiment 19

19. The cyclic peptide of any one of the preceding embodiments, wherein five or fewer of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

### Numbered Embodiment 20

20. The cyclic peptide of any one of the preceding embodiments, wherein five of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

### Numbered Embodiment 21

21. The cyclic peptide of any one of the preceding embodiments, wherein four or fewer of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

### Numbered Embodiment 22

22. The cyclic peptide of any one of the preceding embodiments, wherein four of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

### Numbered Embodiment 23

23. The cyclic peptide of any one of the preceding embodiments, wherein three or fewer of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

### Numbered Embodiment 24

24. The cyclic peptide of any one of the preceding embodiments, wherein three of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

### Numbered Embodiment 25

25. The cyclic peptide of any one of the preceding embodiments, wherein two or fewer of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

### Numbered Embodiment 26

26. The cyclic peptide of any one of the preceding embodiments, wherein two of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

### Numbered Embodiment 27

27. The cyclic peptide of any one of the preceding embodiments, wherein one or fewer of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

### Numbered Embodiment 28

28. The cyclic peptide of any one of the preceding embodiments, wherein one of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, is independently replaced with an amino acid residue that is different from the amino acid residue recited at said position.

### Numbered Embodiment 29

29. The cyclic peptide of any one of the preceding embodiments, wherein none of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

### Numbered Embodiment 30

30. The cyclic peptide of any one of the preceding embodiments, wherein seven or fewer of the amino acid residues at positions Z³, Z⁵, Z⁸, Z¹⁰, Z¹³, Z¹¹ and Z¹⁶, when an amino acid residue is present at the position, are independently replaced with an amino acid residue that is different from the amino acid residue recited at said position.

### Numbered Embodiment 31

31. The cyclic peptide of any one of the preceding embodiments, wherein seven of the amino acid residues at positions Z³, Z⁵, Z⁸, Z¹⁰, Z¹³, Z¹⁵ and Z¹⁶, when an amino acid residue is present at the position, are independently replaced with an amino acid residue that is different from the amino acid residue recited at said position.

### Numbered Embodiment 32

32. The cyclic peptide of any one of the preceding embodiments, wherein six or fewer of the amino acid residues at positions Z³, Z⁵, Z⁸, Z°, Z¹³, Z¹⁵ and Z¹⁶, when an amino acid residue is present at the position, are independently replaced with an amino acid residue that is different from the amino acid residue recited at said position.

### Numbered Embodiment 33

33. The cyclic peptide of any one of the preceding embodiments, wherein six of the amino acid residues at positions Z³, Z⁵, Z⁸, Z¹⁰, Z¹³, Z¹⁵ and Z¹⁶, when an amino acid residue is present at the position, are independently replaced with an amino acid residue that is different from the amino acid residue recited at said position.

### Numbered Embodiment 34

34. The cyclic peptide of any one of the preceding embodiments, wherein five or fewer of the amino acid residues at positions Z³, Z⁵, Z⁸, Z°, Z¹³, Z¹⁵ and Z¹⁶, when an amino acid residue is present at the position, are independently replaced with an amino acid residue that is different from the amino acid residue recited at said position.

### Numbered Embodiment 35

35. The cyclic peptide of any one of the preceding embodiments, wherein five of the amino acid residues at positions Z³, Z⁵, Z⁸, Z¹⁰, Z¹³, Z¹⁵ and Z¹⁶, when an amino acid residue is present at the position, are independently replaced with an amino acid residue that is different from the amino acid residue recited at said position.

### Numbered Embodiment 36

36. The cyclic peptide of any one of the preceding embodiments, wherein four or fewer of the amino acid residues at positions Z³, Z⁵, Z⁸, Z°, Z¹³, Z¹⁵ and Z¹⁶, when an amino acid residue is present at the position, are independently replaced with an amino acid residue that is different from the amino acid residue recited at said position.

### Numbered Embodiment 37

37. The cyclic peptide of any one of the preceding embodiments, wherein four of the amino acid residues at positions Z³, Z⁵, Z⁸, Z¹⁰, Z¹³, Z¹⁵ and Z¹⁶, when an amino acid residue is present at the position, are independently replaced with an amino acid residue that is different from the amino acid residue recited at said position.

### Numbered Embodiment 38

38. The cyclic peptide of any one of the preceding embodiments, wherein three or fewer of the amino acid residues at positions Z³, Z⁵, Z⁸, Z¹⁰, Z¹³, Z¹⁵ and Z¹⁶, when an amino acid residue is present at the position, are independently replaced with an amino acid residue that is different from the amino acid residue recited at said position.

### Numbered Embodiment 39

39. The cyclic peptide of any one of the preceding embodiments, wherein three of the amino acid residues at positions Z³, Z⁵, Z⁸, Z¹⁰, Z¹³, Z¹⁵ and Z¹⁶, when an amino acid residue is present at the position, are independently replaced with an amino acid residue that is different from the amino acid residue recited at said position.

### Numbered Embodiment 40

40. The cyclic peptide of any one of the preceding embodiments, wherein two or fewer of the amino acid residues at positions Z³, Z⁵, Z⁸, Z°, Z¹³, Z¹⁵ and Z¹⁶, when an amino acid residue is present at the position, are independently replaced with an amino acid residue that is different from the amino acid residue recited at said position.

### Numbered Embodiment 41

41. The cyclic peptide of any one of the preceding embodiments, wherein two of the amino acid residues at positions Z³, Z⁵, Z⁸, Z¹⁰, Z¹³, Z¹⁵ and Z¹⁶, when an amino acid residue is present at the position, are independently replaced with an amino acid residue that is different from the amino acid residue recited at said position.

### Numbered Embodiment 42

42. The cyclic peptide of any one of the preceding embodiments, wherein one or fewer of the amino acid residues at positions Z³, Z⁵, Z⁸, Z°, Z¹³, Z¹⁵ and Z¹⁶, when an amino acid residue is present at the position, are independently replaced with an amino acid residue that is different from the amino acid residue recited at said position.

### Numbered Embodiment 43

43. The cyclic peptide of any one of the preceding embodiments, wherein one of the amino acid residues at positions Z³, Z⁵, Z⁸, Z¹⁰, Z¹³, Z¹⁵ and Z¹⁶, when an amino acid residue is present at the position, is independently replaced with an amino acid residue that is different from the amino acid residue recited at said position.

### Numbered Embodiment 44

44. The cyclic peptide of any one of the preceding embodiments, wherein four or fewer of the amino acid residues at positions Z⁵, Z⁸, Z¹⁰, and Z¹³ are independently replaced with an amino acid residue that is different from the amino acid residue recited at said position.

### Numbered Embodiment 45

45. The cyclic peptide of any one of the preceding embodiments, wherein four of the amino acid residues at positions Z⁵, Z⁸, Z¹⁰, and Z¹³ are independently replaced with an amino acid residue that is different from the amino acid residue recited at said position.

### Numbered Embodiment 46

46. The cyclic peptide of any one of the preceding embodiments, wherein three or fewer of the amino acid residues at positions Z⁵, Z⁸, Z¹⁰, and Z¹³ are independently replaced with an amino acid residue that is different from the amino acid residue recited at said position.

### Numbered Embodiment 47

47. The cyclic peptide of any one of the preceding embodiments, wherein three of the amino acid residues at positions Z⁵, Z⁸, Z¹⁰, and Z¹³ are independently replaced with an amino acid residue that is different from the amino acid residue recited at said position.

### Numbered Embodiment 48

48. The cyclic peptide of any one of the preceding embodiments, wherein two or fewer of the amino acid residues at positions Z⁵, Z⁸, Z¹⁰, and Z¹³ are independently replaced with an amino acid residue that is different from the amino acid residue recited at said position.

### Numbered Embodiment 49

49. The cyclic peptide of any one of the preceding embodiments, wherein two of the amino acid residues at positions Z⁵, Z⁸, Z¹⁰, and Z¹³ are independently replaced with an amino acid residue that is different from the amino acid residue recited at said position.

### Numbered Embodiment 50

50. The cyclic peptide of any one of the preceding embodiments, wherein one or fewer of the amino acid residues at positions Z⁵, Z⁸, Z¹⁰, and Z¹³ are independently replaced with an amino acid residue that is different from the amino acid residue recited at said position.

### Numbered Embodiment 51

51. The cyclic peptide of any one of the preceding embodiments, wherein one of the amino acid residues at positions Z⁵, Z⁸, Z¹⁰, and Z¹³ is independently replaced with an amino acid residue that is different from the amino acid residue recited at said position.

### Numbered Embodiment 52

52. The cyclic peptide of any one of the preceding embodiments, wherein the amino acid residue at position Z³, when an amino acid residue is present at the position, is not replaced.

### Numbered Embodiment 53

53. The cyclic peptide of any one of the preceding embodiments, wherein the amino acid residue at position Z⁶ is not replaced.

### Numbered Embodiment 54

54. The cyclic peptide of any one of the preceding embodiments, wherein the amino acid residue at position Z⁷ is not replaced.

### Numbered Embodiment 55

55. The cyclic peptide of any one of the preceding embodiments, wherein the amino acid residue at position Z¹¹ is not replaced.

### Numbered Embodiment 56

56. The cyclic peptide of any one of the preceding embodiments, wherein the amino acid residue at position Z¹², when an amino acid residue is present at the position, is not replaced.

### Numbered Embodiment 57

57. The cyclic peptide of any one of the preceding embodiments, wherein the amino acid residue at position Z¹⁴ is not replaced.

### Numbered Embodiment 58

58. The cyclic peptide of any one of the preceding embodiments, wherein the amino acid residue at position Z¹⁵, when an amino acid residue is present at the position, is not replaced.

### Numbered Embodiment 59

59. The cyclic peptide of any one of the preceding embodiments, wherein the amino acid residue at position Z¹⁶, when an amino acid residue is present at the position, is not replaced.

### Numbered Embodiment 60

60. The cyclic peptide of any one of the preceding embodiments, wherein the amino acid residues at positions Z⁶ and Z¹¹ are not replaced.

### Numbered Embodiment 61

61. The cyclic peptide of any one of the preceding embodiments, wherein the amino acid residues at positions Z⁶ and Z¹⁴ are not replaced.

### Numbered Embodiment 62

62. The cyclic peptide of any one of the preceding embodiments, wherein the amino acid residues at positions Z¹¹ and Z¹⁴ are not replaced.

### Numbered Embodiment 63

63. The cyclic peptide of any one of the preceding embodiments, wherein the amino acid residues at positions Z⁶, Z¹¹ and Z¹⁴ are not replaced.

### Numbered Embodiment 64

64. The cyclic peptide of any one of the preceding embodiments, wherein the amino acid residues at positions Z⁶, Z⁷, Z¹¹ and Z¹⁴ are not replaced.

### Numbered Embodiment 65

65. The cyclic peptide of any one of the preceding embodiments, wherein the amino acid residues at positions Z⁶, Z¹¹, Z¹² and Z¹⁴, when an amino acid residue is present at the position, are not replaced.

### Numbered Embodiment 66

66. The cyclic peptide of any one of the preceding embodiments, wherein the amino acid residues at positions Z⁶, Z⁷, Z¹¹, Z¹² and Z¹⁴, when an amino acid residue is present at the position, are not replaced.

### Numbered Embodiment 67

67. The cyclic peptide of any one of the preceding embodiments, wherein the amino acid residues at positions Z³, Z⁶, Z⁷, Z¹¹, Z¹² and Z¹⁴, when an amino acid residue is present at the position, are not replaced.

### Numbered Embodiment 68

68. The cyclic peptide of any one of the preceding embodiments, wherein the amino acid residues at positions Z⁶, Z⁷, Z¹¹, Z¹², Z¹⁴ and Z¹⁵, when an amino acid residue is present at the position, are not replaced.

### Numbered Embodiment 69

69. The cyclic peptide of any one of the preceding embodiments, wherein the amino acid residues at positions Z³, Z⁶, Z⁷, Z¹¹, Z¹², Z¹⁴ and Z¹⁵, when an amino acid residue is present at the position, are not replaced.

### Numbered Embodiment 70

70. The cyclic peptide of any one of the preceding embodiments, wherein the amino acid residues at positions Z⁶, Z⁷, Z¹¹, Z¹², Z¹⁴ and Z¹⁶, when an amino acid residue is present at the position, are not replaced.

### Numbered Embodiment 71

71. The cyclic peptide of any one of the preceding embodiments, wherein the amino acid residues at positions Z³, Z⁶, Z⁷, Z¹¹, Z¹², Z¹⁴ and Z¹⁶, when an amino acid residue is present at the position, are not replaced.

### Numbered Embodiment 72

72. The cyclic peptide of any one of the preceding embodiments, wherein the amino acid residues at positions Z⁶, Z⁷, Z¹¹, Z¹², Z¹⁴, Z¹⁵ and Z¹⁶, when an amino acid residue is present at the position, are not replaced.

### Numbered Embodiment 73

73. The cyclic peptide of any one of the preceding embodiments, wherein the amino acid residues at positions Z³, Z⁶, Z⁷, Z¹¹, Z¹², Z¹⁴, Z¹¹ and Z¹⁶, when an amino acid residue is present at the position, are not replaced.

### Numbered Embodiment 74

74. The cyclic peptide of any one of the preceding embodiments, wherein three or fewer of the amino acid residues at positions Z³, Z¹², Z¹⁵ and Z¹⁶ are absent.

### Numbered Embodiment 75

75. The cyclic peptide of any one of the preceding embodiments, wherein three of the amino acid residues at positions Z³, Z¹², Z¹⁵ and Z¹⁶ are absent.

### Numbered Embodiment 76

76. The cyclic peptide of any one of the preceding embodiments, wherein two or fewer of the amino acid residues at positions Z³, Z¹², Z¹⁵ and Z¹⁶ are absent.

### Numbered Embodiment 77

77. The cyclic peptide of any one of the preceding embodiments, wherein two of the amino acid residues at positions Z³, Z¹², Z¹⁵ and Z¹⁶ are absent.

### Numbered Embodiment 78

78. The cyclic peptide of any one of the preceding embodiments, wherein one or fewer of the amino acid residues at positions Z³, Z¹², Z¹⁵ and Z¹⁶ are absent.

### Numbered Embodiment 79

79. The cyclic peptide of any one of the preceding embodiments, wherein one of the amino acid residues at positions Z³, Z¹², Z¹⁵ and Z¹⁶ is absent.

### Numbered Embodiment 80

80. The cyclic peptide of any one of the preceding embodiments, wherein none of the amino acid residues at positions Z³, Z¹², Z¹⁵ and Z¹⁶ are absent.

### Numbered Embodiment 81

81. The cyclic peptide of any one of the preceding embodiments, wherein the amino acid residue at position Z³ is absent and the amino acid residues at positions Z¹², Z¹⁵ and Z¹⁶ are present.

### Numbered Embodiment 82

82. The cyclic peptide of any one of the preceding embodiments, wherein the amino acid residue at position Z¹² is absent and the amino acid residues at positions Z³, Z¹⁵ and Z¹⁶ are present.

### Numbered Embodiment 83

83. The cyclic peptide of any one of the preceding embodiments, wherein the amino acid residue at position Z¹⁵ is absent and the amino acid residues at positions Z³, Z¹² and Z¹⁶ are present.

### Numbered Embodiment 84

84. The cyclic peptide of any one of the preceding embodiments, wherein the amino acid residue at position Z¹⁶ is absent and the amino acid residues at positions Z³, Z¹² and Z¹⁵ are present.

### Numbered Embodiment 85

85. The cyclic peptide of any one of the preceding embodiments, wherein the amino acid residues at positions Z³ and Z¹⁶ are absent and the amino acid residues at positions Z¹² and Z⁵ are present.

### Numbered Embodiment 86

86. The cyclic peptide of any one of the preceding embodiments, wherein the amino acid residues at positions Z³, Z¹⁵ and Z¹⁶ are absent and the amino acid residue at position Z¹² is present.

### Numbered Embodiment 87

87. The cyclic peptide of any one of the preceding embodiments, wherein at least one of R^{NT}, when present, and/or R^{CT}, when present, and/or the amino acid residues at positions Z³, Z⁵, Z⁶, Z⁷, Z⁸, Z¹⁰, Z¹¹, Z¹², Z¹³, Z¹⁴, Z¹⁵ and/or Z¹⁶, when an amino acid residue is present at the position, each independently comprise(s) at least one quaternary amine.

### Numbered Embodiment 88

88. The cyclic peptide of any one of the preceding embodiments, wherein at least one of R^{NT}, when present, and/or R^{CT}, when present, and/or the amino acid residues at positions Z³, Z⁵, Z⁶, Z⁷, Z⁸, Z¹⁰, Z¹¹, Z¹², Z¹³, Z¹⁴, Z¹⁵ and/or Z¹⁶, when an amino acid residue is present at the position, each independently comprise(s) a quaternary amine.

### Numbered Embodiment 89

89. The cyclic peptide of any one of the preceding embodiments, wherein at least one of the amino acid residues at positions Z³, Z⁵, Z⁶, Z⁷, Z⁸, Z¹⁰, Z¹¹, Z¹², Z¹³, Z¹⁴, Z¹¹ and/or Z¹⁶, when an amino acid residue is present at the position, each independently comprise(s) at least one quaternary amine.

### Numbered Embodiment 90

90. The cyclic peptide of any one of the preceding embodiments, wherein at least one of the amino acid residues at positions Z³, Z⁵, Z⁶, Z⁷, Z⁸, Z¹⁰, Z¹¹, Z¹², Z¹³, Z¹⁴, Z¹¹ and/or Z¹⁶, when an amino acid residue is present at the position, each independently comprise(s) a quaternary amine.

### Numbered Embodiment 91

91. The cyclic peptide of any one of the preceding embodiments, wherein at least one of R^{NT}, when present, and/or R^{CT}, when present, and/or the amino acid residues at positions Z³, Z⁵, Z⁸, Z¹⁰, Z¹² and/or Z¹³, when an amino acid residue is present at the position, each independently comprise(s) at least one quaternary amine.

### Numbered Embodiment 92

92. The cyclic peptide of any one of the preceding embodiments, wherein at least one of R^{NT}, when present, and/or R^{CT}, when present, and/or the amino acid residues at positions Z³, Z⁵, Z⁸, Z¹⁰, Z¹², and/or Z¹³, when an amino acid residue is present at the position, each independently comprise(s) a quaternary amine.

### Numbered Embodiment 93

93. The cyclic peptide of any one of the preceding embodiments, wherein at least one of R^{NT}, when present, and/or the amino acid residues at positions Z³, Z⁵, Z⁸, Z¹⁰, and/or Z¹³, when an amino acid residue is present at the position, each independently comprise(s) at least one quaternary amine.

### Numbered Embodiment 94

94. The cyclic peptide of any one of the preceding embodiments, wherein at least one of R^{NT}, when present, and/or the amino acid residues at positions Z³, Z⁵, Z⁸, Z¹⁰, and/or Z¹³, when an amino acid residue is present at the position, each independently comprise(s) a quaternary amine.

### Numbered Embodiment 95

95. The cyclic peptide of any one of the preceding embodiments, wherein R^{NT} and the amino acid residues at positions Z⁵ and Z¹⁰ each independently comprise at least one quaternary amine.

### Numbered Embodiment 96

96. The cyclic peptide of any one of the preceding embodiments, wherein R^{NT} and the amino acid residues at positions Z⁵ and Z¹⁰ each independently comprise a quaternary amine.

### Numbered Embodiment 97

97. The cyclic peptide of any one of the preceding embodiments, wherein R^{NT} and the amino acid residues at positions Z⁸ and Z¹⁰ each independently comprise at least one quaternary amine.

### Numbered Embodiment 98

98. The cyclic peptide of any one of the preceding embodiments, wherein R^{NT} and the amino acid residues at positions Z⁸ and Z¹⁰ each independently comprise a quaternary amine.

### Numbered Embodiment 99

99. The cyclic peptide of any one of the preceding embodiments, wherein R^{NT} and the amino acid residues at positions Z⁸ and Z¹³ each independently comprise at least one quaternary amine.

### Numbered Embodiment 100

100. The cyclic peptide of any one of the preceding embodiments, wherein R^{NT} and the amino acid residues at positions Z⁸ and Z¹³ each independently comprise a quaternary amine.

### Numbered Embodiment 101

101. The cyclic peptide of any one of the preceding embodiments, wherein the amino acid residues at positions Z⁸, Z¹⁰ and Z¹³ each independently comprise at least one quaternary amine.

### Numbered Embodiment 102

102. The cyclic peptide of any one of the preceding embodiments, wherein the amino acid residues at positions Z⁸, Z¹⁰ and Z¹³ each independently comprise a quaternary amine.

### Numbered Embodiment 103

103. The cyclic peptide of any one of the preceding embodiments, wherein R^{NT} and the amino acid residue at position Z³ each independently comprise at least one quaternary amine.

### Numbered Embodiment 104

104. The cyclic peptide of any one of the preceding embodiments, wherein R^{NT} and the amino acid residue at position Z³ each independently comprise a quaternary amine.

### Numbered Embodiment 105

105. The cyclic peptide of any one of the preceding embodiments, wherein R^{NT} and R^{CT}each independently comprise at least one quaternary amine.

### Numbered Embodiment 106

106. The cyclic peptide of any one of the preceding embodiments, wherein R^{NT} and R^{CT}each comprise a quaternary amine.

### Numbered Embodiment 107

107. The cyclic peptide of any one of the preceding embodiments, wherein R^{NT} and the amino acid residue at position Z⁸ each independently comprise at least one quaternary amine.

### Numbered Embodiment 108

108. The cyclic peptide of any one of the preceding embodiments, wherein R^{NT} and the amino acid residue at position Z⁸ each independently comprise a quaternary amine.

### Numbered Embodiment 109

109. The cyclic peptide of any one of the preceding embodiments, wherein R^{NT} and the amino acid residue at position Z¹⁰ each independently comprise at least one quaternary amine.

### Numbered Embodiment 110

110. The cyclic peptide of any one of the preceding embodiments, wherein R^{NT} and the amino acid residue at position Z¹⁰ each independently comprise a quaternary amine.

### Numbered Embodiment 111

111. The cyclic peptide of any one of the preceding embodiments, wherein the amino acid residues at positions Z³ and Z⁸ each independently comprise at least one quaternary amine.

### Numbered Embodiment 112

112. The cyclic peptide of any one of the preceding embodiments, wherein the amino acid residues at positions Z³ and Z⁸ each independently comprise a quaternary amine.

### Numbered Embodiment 113

113. The cyclic peptide of any one of the preceding embodiments, wherein the amino acid residues at positions Z³ and Z¹⁰ each independently comprise at least one quaternary amine.

### Numbered Embodiment 114

114. The cyclic peptide of any one of the preceding embodiments, wherein the amino acid residues at positions Z³ and Z¹⁰ each independently comprise a quaternary amine.

### Numbered Embodiment 115

115. The cyclic peptide of any one of the preceding embodiments, wherein the amino acid residues at positions Z⁵ and Z¹⁰ each independently comprise at least one quaternary amine.

### Numbered Embodiment 116

116. The cyclic peptide of any one of the preceding embodiments, wherein the amino acid residues at positions Z⁵ and Z¹⁰ each independently comprise a quaternary amine.

### Numbered Embodiment 117

117. The cyclic peptide of any one of the preceding embodiments, wherein the amino acid residues at positions Z⁸ and Z¹⁰ each independently comprise at least one quaternary amine.

### Numbered Embodiment 118

118. The cyclic peptide of any one of the preceding embodiments, wherein the amino acid residues at positions Z⁸ and Z¹⁰ each independently comprise a quaternary amine.

### Numbered Embodiment 119

119. The cyclic peptide of any one of the preceding embodiments, wherein the amino acid residues at positions Z⁸ and Z¹³ each independently comprise at least one quaternary amine.

### Numbered Embodiment 120

120. The cyclic peptide of any one of the preceding embodiments, wherein the amino acid residues at positions Z⁸ and Z¹³ each independently comprise a quaternary amine.

### Numbered Embodiment 121

121. The cyclic peptide of any one of the preceding embodiments, wherein the amino acid residues at positions Z¹⁰ and Z¹³ each independently comprise at least one quaternary amine.

### Numbered Embodiment 122

122. The cyclic peptide of any one of the preceding embodiments, wherein the amino acid residues at positions Z¹⁰ and Z¹³ each independently comprise a quaternary amine.

### Numbered Embodiment 123

123. The cyclic peptide of any one of the preceding embodiments, wherein R^{NT} comprises at least one quaternary amine.

### Numbered Embodiment 124

124. The cyclic peptide of any one of the preceding embodiments, wherein R^{NT} comprises a quaternary amine.

### Numbered Embodiment 125

125. The cyclic peptide of any one of the preceding embodiments, wherein R^{CT} comprises at least one quaternary amine.

### Numbered Embodiment 126

126. The cyclic peptide of any one of the preceding embodiments, wherein R^{CT} comprises a quaternary amine.

### Numbered Embodiment 127

127. The cyclic peptide of any one of the preceding embodiments, wherein the amino acid residue at position Z³ comprises at least one quaternary amine.

### Numbered Embodiment 128

128. The cyclic peptide of any one of the preceding embodiments, wherein the amino acid residue at position Z³ comprises a quaternary amine.

### Numbered Embodiment 129

129. The cyclic peptide of any one of the preceding embodiments, wherein the amino acid residue at position Z⁵ comprises at least one quaternary amine.

### Numbered Embodiment 130

130. The cyclic peptide of any one of the preceding embodiments, wherein the amino acid residue at position Z⁵ comprises a quaternary amine.

### Numbered Embodiment 131

131. The cyclic peptide of any one of the preceding embodiments, wherein the amino acid residue at position Z⁸ comprises at least one quaternary amine.

### Numbered Embodiment 132

132. The cyclic peptide of any one of the preceding embodiments, wherein the amino acid residue at position Z⁸ comprises a quaternary amine.

### Numbered Embodiment 133

133. The cyclic peptide of any one of the preceding embodiments, wherein the amino acid residue at position Z¹⁰ comprises at least one quaternary amine.

### Numbered Embodiment 134

134. The cyclic peptide of any one of the preceding embodiments, wherein the amino acid residue at position Z¹⁰ comprises a quaternary amine.

### Numbered Embodiment 135

135. The cyclic peptide of any one of the preceding embodiments, wherein the amino acid residue at position Z¹² comprises at least one quaternary amine.

### Numbered Embodiment 136

136. The cyclic peptide of any one of the preceding embodiments, wherein the amino acid residue at position Z¹² comprises a quaternary amine.

### Numbered Embodiment 137

137. The cyclic peptide of any one of the preceding embodiments, wherein the amino acid residue at position Z¹³ comprises at least one quaternary amine.

### Numbered Embodiment 138

138. The cyclic peptide of any one of the preceding embodiments, wherein the amino acid residue at position Z¹³ comprises a quaternary amine.

### Numbered Embodiment 139

139. The cyclic peptide of any one of the preceding embodiments, wherein the at least one quaternary amine is one or two quaternary amines.

### Numbered Embodiment 140

140. The cyclic peptide of any one of the preceding embodiments, wherein each quaternary amine is independently selected from the group consisting of:

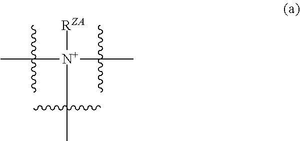

wherein each R^{ZA} is independently selected from the group consisting of: optionally substituted (C₁-C₂₀) alkyl and optionally substituted (C₁-C₂₀) heteroalkyl;

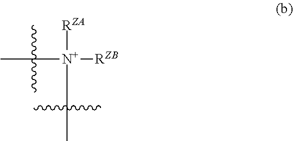

wherein each R^{ZA} and R^{ZB} is independently selected from the group consisting of: optionally substituted (C₁-C₂₀) alkyl and optionally substituted (C₁-C₂₀) heteroalkyl;

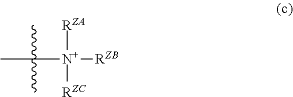

wherein
(c)(i) each R^{Z}A, R^{ZB} and R^{ZC} is independently selected from the group consisting of: optionally substituted (C₁-C₂₀) alkyl and optionally substituted (C₁-C₂₀) heteroalkyl,
(c)(ii) each R^{ZA} is independently selected from the group consisting of: optionally substituted (C₁-C₂₀) alkyl and optionally substituted (C₁-C₂₀) heteroalkyl, and each R^{ZB} and R^{ZC} come together with the N atom to which they are attached to form an optionally substituted (CH₃—C₁₄) heterocyclic ring, or
(c)(iii) each R^{Z}A, R^{ZB} and R^{ZC} come together with the N atom to which they are attached to form an optionally substituted (C₅-C₁₀) bicyclic heterocyclic ring;

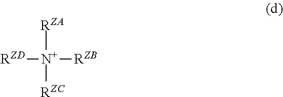

wherein
(d)(i) each R^{ZA} and R^{ZB} is independently selected from the group consisting of: optionally substituted (C₁-C₂₀) alkyl and optionally substituted (C₁-C₂₀) heteroalkyl, and each R^{ZC} and R^{ZD} come together with the N atom to which they are attached to form an optionally substituted (CH₃—C₁₄) heterocyclyl, or
(d)(ii) each R^{ZA} and R^{ZB} come together with the N atom to which they are attached to form an optionally substituted (CH₃—C₁₄) heterocyclyl, and each R^{ZC} and R^{ZD} come together with the N atom to which they are attached to form an optionally substituted (CH₃—C₁₄) heterocyclyl, or
(d)(iii) each R^{ZA} and R^{ZB} come together with the N atom to which they are attached to form an optionally substituted (CH₃—C₁₄) heterocyclyl, and each R^{ZC} and R^{ZD} come together with the N atom to which they are attached to form an optionally substituted (CH₃—C₁₄) heterocyclic ring;

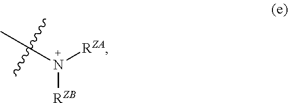

wherein each R^{ZA} and R^{ZB} come together with the N atom to which they are attached to form an optionally substituted (C₅-C₁₄) heteroaromatic ring, and

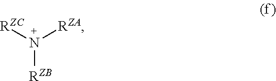

wherein each R^{ZA} is independently selected from the group consisting of: optionally substituted (C₁-C₂₀) alkyl and optionally substituted (C₁-C₂₀) heteroalkyl, and each R^{ZB} and R^{ZC} come together with the N atom to which they are attached to form an optionally substituted (C₅-C₁₄) heteroaryl.

### Numbered Embodiment 141

141. The cyclic peptide of any one of the preceding embodiments, wherein each quaternary amine is independently selected from the group consisting of:

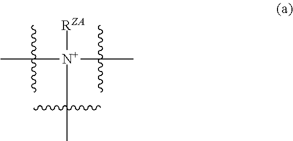

wherein each R^{ZA} is independently selected from the group consisting of: optionally substituted (C₁-C₁₆) alkyl and optionally substituted (C₁-C₈) heteroalkyl;

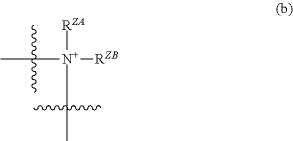

wherein each R^{ZA} and R^{ZB} is independently selected from the group consisting of: optionally substituted (C₁-C₁₆) alkyl and optionally substituted (C₁-C₈) heteroalkyl;

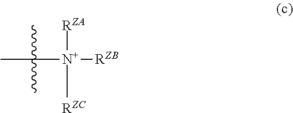

(c)(i) each R^{ZA}, R^{ZB} and R^{ZC} is independently selected from the group consisting of: optionally substituted (C₁-C₁₆) alkyl and optionally substituted (C₁-C₈) heteroalkyl,
(c)(ii) each R^{ZA} is independently selected from the group consisting of: optionally substituted (C₁-C₁₆) alkyl and optionally substituted (C₁-C₈) heteroalkyl, and each R^{ZB} and R^{ZC} come together with the N atom to which they are attached to form an optionally substituted (C₆) heterocyclic ring, or
(c)(iii) each R^{ZA}, R^{ZB} and R^{ZC} come together with the N atom to which they are attached to form an optionally substituted (C₈) bicyclic heterocyclic ring;

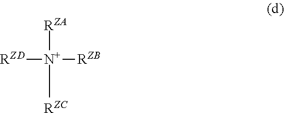

wherein
(d)(i) each R^{ZA} and R^{ZB} is independently selected from the group consisting of: optionally substituted (C₁-C₁₆) alkyl and optionally substituted (C₁-C₈) heteroalkyl, and each R^{ZC} and R^{ZD} come together with the N atom to which they are attached to form an optionally substituted (C₆) heterocyclyl, or
(d)(ii) each R^{ZA} and R^{ZB} come together with the N atom to which they are attached to form an optionally substituted (C₆) heterocyclyl, and each R^{ZC} and R^{ZD}come together with the N atom to which they are attached to form an optionally substituted (C₆) heterocyclyl, or
(d)(iii) each R^{ZA} and R^{ZB} come together with the N atom to which they are attached to form an optionally substituted (C₆) heterocyclyl, and each R^{ZC} and R^{ZD}come together with the N atom to which they are attached to form an optionally substituted (C₆) heterocyclic ring;

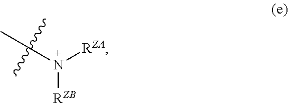

wherein each R^{ZA} and R^{ZB} come together with the N atom to which they are attached to form an optionally substituted (C₆) heteroaromatic ring; and

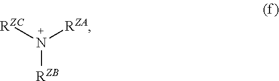

wherein each R^{ZA} is independently selected from the group consisting of: optionally substituted (C₁-C₁₆) alkyl and optionally substituted (C₁-C₈) heteroalkyl, and each R^{ZB} and R^{ZC} come together with the N atom to which they are attached to form an optionally substituted (C₆) heteroaryl.

### Numbered Embodiment 142

142. The cyclic peptide of any one of the preceding embodiments, wherein each quaternary amine is independently selected from the group consisting of:

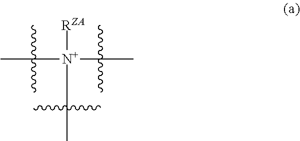

wherein each R^{ZA} is independently selected from the group consisting of: optionally substituted (C₁-C₁₆) alkyl and optionally substituted (C₁-C₈) heteroalkyl;

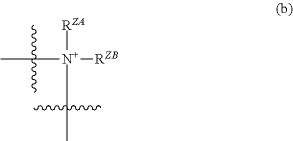

wherein each R^{ZA} and R^{ZB} is independently selected from the group consisting of: optionally substituted (C₁-C₁₆) alkyl and optionally substituted (C₁-C₈) heteroalkyl;

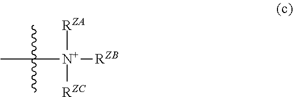

wherein
(c)(i) each R^{ZA}, R^{ZB} and R^{ZC} is independently selected from the group consisting of: optionally substituted (C₁-C₁₆) alkyl and optionally substituted (C₁-C₈) heteroalkyl,
(c)(ii) each R^{ZA} is independently selected from the group consisting of: optionally substituted (C₁-C₃) alkyl and optionally substituted (C₁-C₃) heteroalkyl, and each R^{ZB} and R^{ZC} come together with the N atom to which they are attached to form an optionally substituted (C₆) heterocyclic ring, or
(c)(iii) each R^{ZA}, R^{ZB} and R^{ZC} come together with the N atom to which they are attached to form an optionally substituted (C₈) bicyclic heterocyclic ring;

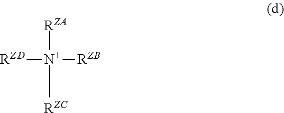

wherein
(d)(i) each R^{ZA} and R^{ZB} is independently selected from the group consisting of: optionally substituted (C₁-C₃) alkyl and optionally substituted (C₁-C₃) heteroalkyl, and each R^{ZC} and R^{ZD} come together with the N atom to which they are attached to form an optionally substituted (C₆) heterocyclyl, or
(d)(ii) each R^{ZA} and R^{ZB} come together with the N atom to which they are attached to form an optionally substituted (C₆) heterocyclyl, and each R^{ZC} and R^{ZD}come together with the N atom to which they are attached to form an optionally substituted (C₆) heterocyclyl, or
(d)(iii) each R^{ZA} and R^{ZB} come together with the N atom to which they are attached to form an optionally substituted (C₆) heterocyclyl, and each R^{ZC} and R^{ZD}come together with the N atom to which they are attached to form an optionally substituted (C₆) heterocyclic ring;

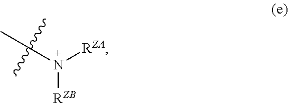

wherein each R^{ZA} and R^{ZB} come together with the N atom to which they are attached to form an optionally substituted (C₆) heteroaromatic ring; and

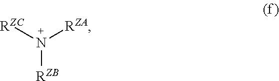

wherein each R^{ZA} is independently selected from the group consisting of: optionally substituted (C₁-C₃) alkyl and optionally substituted (C₁-C₃) heteroalkyl, and each R^{ZB} and R^{ZC} come together with the N atom to which they are attached to form an optionally substituted (C) heteroaryl.

### Numbered Embodiment 143

143. The cyclic peptide of any one of the preceding embodiments, wherein when each R^{ZA}R^{ZB} and/or R^{ZC}, when present, is/are an optionally substituted alkyl, each optionally substituted alkyl is unsubstituted.

### Numbered Embodiment 144

144. The cyclic peptide of any one of the preceding embodiments, wherein when each R^{ZA}R^{ZB} and/or R^{ZC}, when present, is/are an optionally substituted heteroalkyl, each optionally substituted heteroalkyl is unsubstituted.

### Numbered Embodiment 145

145. The cyclic peptide of any one of the preceding embodiments, wherein when each R^{ZB} and R^{ZC} come together with the N atom to which they are attached to form an optionally substituted heterocyclic ring, the optionally substituted heterocyclic ring is unsubstituted or substituted with one or more substitutents independently selected from the group consisting of Fluoro and (C₁-C₃) alkyl.

### Numbered Embodiment 146

146. The cyclic peptide of any one of the preceding embodiments, wherein when each R^{ZA}R^{ZB} and R^{ZC} come together with the N atom to which they are attached to form an optionally substituted bicyclic heterocyclic ring, the optionally substituted bicyclic heterocyclic ring is unsubstituted.

### Numbered Embodiment 147

147. The cyclic peptide of any one of the preceding embodiments, wherein when each R^{ZA} and R^{ZB} come together with the N atom to which they are attached to form an optionally substituted heterocyclyl, the optionally substituted heterocyclyl is unsubstituted.

### Numbered Embodiment 148

148. The cyclic peptide of any one of the preceding embodiments, wherein when each R^{ZB} and R^{ZC} come together with the N atom to which they are attached to form an optionally substituted heteroaryl, the optionally substituted heteroaryl is unsubstituted.

### Numbered Embodiment 149

149. The cyclic peptide of any one of the preceding embodiments, wherein when each R^{ZC} and R^{ZD} come together with the N atom to which they are attached to form an optionally substituted heterocyclyl, the optionally substituted heterocyclyl is unsubstituted.

### Numbered Embodiment 150

150. The cyclic peptide of any one of the preceding embodiments, wherein when each R^{ZC} and R^{ZD} come together with the N atom to which they are attached to form an optionally substituted heterocyclic ring, the optionally substituted heterocyclic ring is unsubstituted.

### Numbered Embodiment 151

151. The cyclic peptide of any one of the preceding embodiments, wherein when each R^{ZA} and R^{ZB} come together with the N atom to which they are attached to form an optionally substituted heteroaromatic ring, the optionally substituted heteroaromatic ring is unsubstituted.

### Numbered Embodiment 152

152. The cyclic peptide of any one of the preceding embodiments, wherein each quaternary amine is independently selected from the group consisting of:

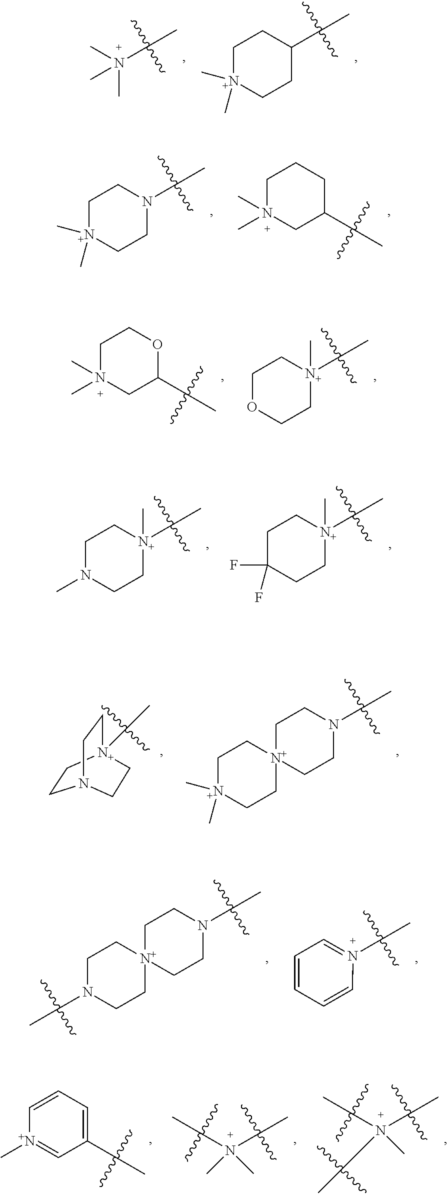

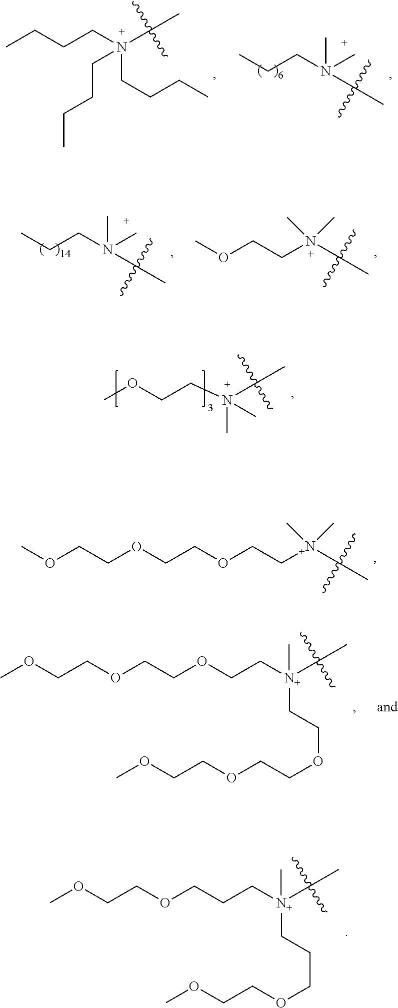

### Numbered Embodiment 153

153. The cyclic peptide of any one of the preceding embodiments, wherein each quaternary amine has an AN⁻counterion that is the same for each quaternary amine and is selected from the group consisting of: acetate, adipate, benzoate, benzenesulfonate, citrate, decanoate, chloride, lactate, maleate, methanesulfonate, oxalate, pivalate, propionate, succinate, sulfate, tartrate, or trifluoroacetate.

### Numbered Embodiment 154

154. The cyclic peptide of any one of the preceding embodiments, wherein at least one of the amino acid residues at positions Z³, Z⁵, Z⁶, Z⁷, Z⁸, Z¹⁰, Z¹¹, Z¹², Z¹³, Z¹⁴, Z¹⁵ and/or Z¹⁶, when an amino acid residue is present at the position, each independently comprise(s) at least one masked amine and/or masked amide.

### Numbered Embodiment 155

155. The cyclic peptide of any one of the preceding embodiments, wherein at least one of the amino acid residues at positions Z³, Z⁵, Z⁶, Z, Z⁸° Z¹⁰, Z¹¹, Z¹², Z¹³, Z¹⁴, Z¹⁵ and/or Z¹⁶, when an amino acid residue is present at the position, each independently comprise(s) a masked amine and/or masked amide.

### Numbered Embodiment 156

156. The cyclic peptide of any one of the preceding embodiments, wherein at least one of the amino acid residues at positions Z⁵, Z⁷, Z⁸, Z¹⁰, Z¹³, Z¹⁴, Z¹⁵ and/or Z¹⁶, when an amino acid residue is present at the position, each independently comprise(s) at least one masked amine and/or masked amide.

### Numbered Embodiment 157

157. The cyclic peptide of any one of the preceding embodiments, wherein at least one of the amino acid residues at positions Z⁵, Z⁷, Z⁸, Z¹⁰, Z¹³, Z¹⁴, Z¹⁵ and/or Z¹⁶, when an amino acid residue is present at the position, each independently comprise(s) a masked amine and/or masked amide.

### Numbered Embodiment 158

158. The cyclic peptide of any one of the preceding embodiments, wherein at least one of the amino acid residues at positions Z⁵, Z⁸, and/or Z¹³, each independently comprise(s) at least one masked amine and/or masked amide.

### Numbered Embodiment 159

159. The cyclic peptide of any one of the preceding embodiments, wherein at least one of the amino acid residues at positions Z⁵, Z⁸, and/or Z¹³, each independently comprise(s) a masked amine and/or masked amide.

### Numbered Embodiment 160

160. The cyclic peptide of any one of the preceding embodiments, wherein the amino acid residue at position Z⁵ comprises at least one masked amine and/or masked amide.

### Numbered Embodiment 161

161. The cyclic peptide of any one of the preceding embodiments, wherein the amino acid residue at position Z⁵ comprises a masked amine and/or masked amide.

### Numbered Embodiment 162

162. The cyclic peptide of any one of the preceding embodiments, wherein the amino acid residue at position Z⁸ comprises at least one masked amine and/or masked amide.

### Numbered Embodiment 163

163. The cyclic peptide of any one of the preceding embodiments, wherein the amino acid residue at position Z⁸ comprises a masked amine and/or masked amide.

### Numbered Embodiment 164

164. The cyclic peptide of any one of the preceding embodiments, wherein the amino acid residue at position Z¹³ comprises at least one masked amine and/or masked amide.

### Numbered Embodiment 165

165. The cyclic peptide of any one of the preceding embodiments, wherein the amino acid residue at position Z¹³ comprises a masked amine and/or masked amide.

### Numbered Embodiment 166

166. The cyclic peptide of any one of the preceding embodiments, wherein at least one of the masked amine(s) is an amine in a backbone amide, wherein each amine in a backbone amide is independently substituted with (C₁-C₃) alkyl.

### Numbered Embodiment 167

167. The cyclic peptide of any one of the preceding embodiments, wherein at least one of the masked amine(s) and/or masked amide is present in the side chain of the amino acid residue comprising said masked amine and/or masked amide.

### Numbered Embodiment 168

168. The cyclic peptide of any one of the preceding embodiments, wherein at least one of the masked amine(s) and/or masked amide(s) is independently selected from the group consisting of:

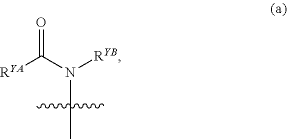

wherein each R^{YA} is independently selected from the group consisting of: optionally substituted (C₁-C₁₀) alkyl and optionally substituted (C₁-C₂₀) heteroalkyl, and each R^{YB} is independently selected from the group consisting of: hydrogen, optionally substituted (C₁-C₁₀) alkyl and optionally substituted (C₁-C₂₀) heteroalkyl;

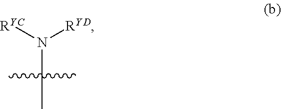

wherein each R^{YC} is independently selected from the group consisting of: optionally substituted (C₁-C₁₀) alkyl and optionally substituted (C₁-C₂₀) heteroalkyl, and each R^{YD} is independently selected from the group consisting of: hydrogen, optionally substituted (C₁-C₁₀) alkyl and optionally substituted (C₁-C₂₀) heteroalkyl;

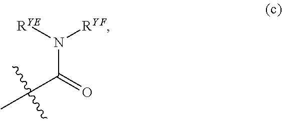

wherein each R^{YE} is independently selected from the group consisting of: optionally substituted (C₁-C₁₀) alkyl and optionally substituted (C₁-C₂₀) heteroalkyl, and each R^{YF} is independently selected from the group consisting of: hydrogen, optionally substituted (C₁-C₁₀) alkyl and optionally substituted (C₁-C₂₀) heteroalkyl; and

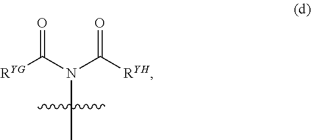

wherein each R^{YG} and R^{YH} is independently selected from the group consisting of: optionally substituted (C₁-C₁₀) alkyl and optionally substituted (C₁-C₂₀) heteroalkyl.

### Numbered Embodiment 169

169. The cyclic peptide of any one of the preceding embodiments, wherein at least one of the masked amine(s) and/or masked amide(s) is independently selected from the group consisting of:

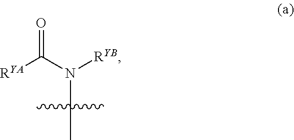

wherein each R^{YA} is independently selected from the group consisting of: optionally substituted (C₁-C₅) alkyl and optionally substituted (C₁-C₁₅) heteroalkyl, and each R^{YB} is independently selected from the group consisting of: hydrogen, optionally substituted (C₁-C₅) alkyl and optionally substituted (C₁-C₁₅) heteroalkyl;

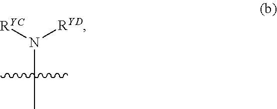

wherein each R^{YC} is independently selected from the group consisting of: optionally substituted (C₁-C₅) alkyl and optionally substituted (C₁-C₁₅) heteroalkyl, and each R^{YD} is independently selected from the group consisting of: hydrogen, optionally substituted (C₁-C₅) alkyl and optionally substituted (C₁-C₁₅) heteroalkyl;

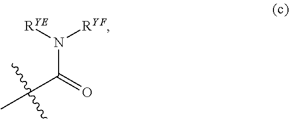

wherein each R^{YE} is independently selected from the group consisting of: optionally substituted (C₁-C₅) alkyl and optionally substituted (C₁-C₁₅) heteroalkyl, and each R^{YF} is independently selected from the group consisting of: hydrogen, optionally substituted (C₁-C₅) alkyl and optionally substituted (C₁-C₁₅) heteroalkyl; and

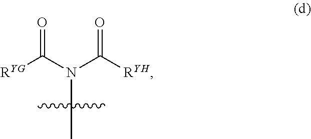

wherein each R^{YG} and R^{YH} is independently selected from the group consisting of: optionally substituted (C₁-C₅) alkyl and optionally substituted (C₁-C₁₅) heteroalkyl.

### Numbered Embodiment 170

170. The cyclic peptide of any one of the preceding embodiments, wherein when each R^{YA}R^{YB}, R^{Y}C, R^{Y}D, R^{YE}, R^{YF}, R^{YG} and/or R^{YH} is optionally substituted alkyl, each optionally substituted alkyl is independently unsubstituted or substituted with one or more substitutents independently selected from the group consisting of: —CO₂H and a quaternary amine of any one of the preceding embodiments, for example, —N⁺(CH₃)₃.

### Numbered Embodiment 171

171. The cyclic peptide of any one of the preceding embodiments, wherein when each R^{YA}R^{YB}, R^{Y}C, R^{Y}D, R^{YE}, R^{YF}, R^{YG} and/or R^{YH} is optionally substituted heteroalkyl, each optionally substituted heteroalkyl is independently unsubstituted or substituted with one or more substitutents independently selected from the group consisting of: —CO₂H and a quaternary amine of any one of the preceding embodiments, for example, —N⁺(CH₃)₃.

### Numbered Embodiment 172

172. The cyclic peptide of any one of the preceding embodiments, wherein at least one of the masked amine(s) and/or masked amide(s) is independently selected from the group consisting of:

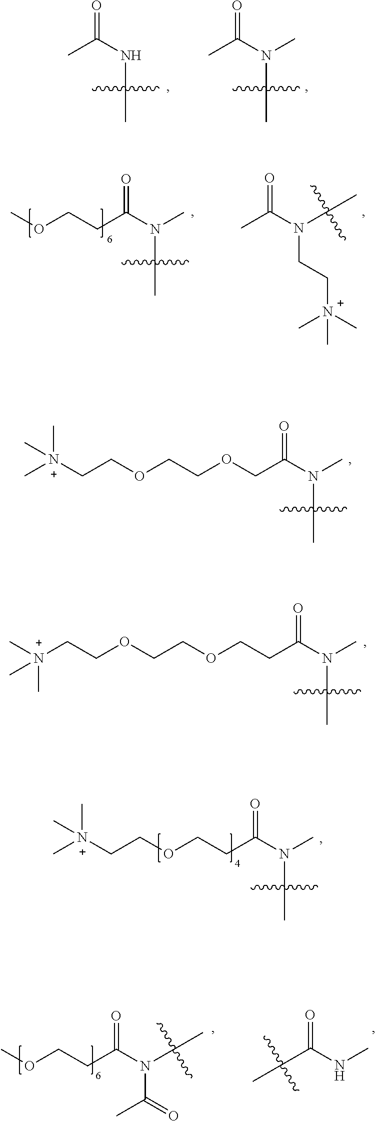

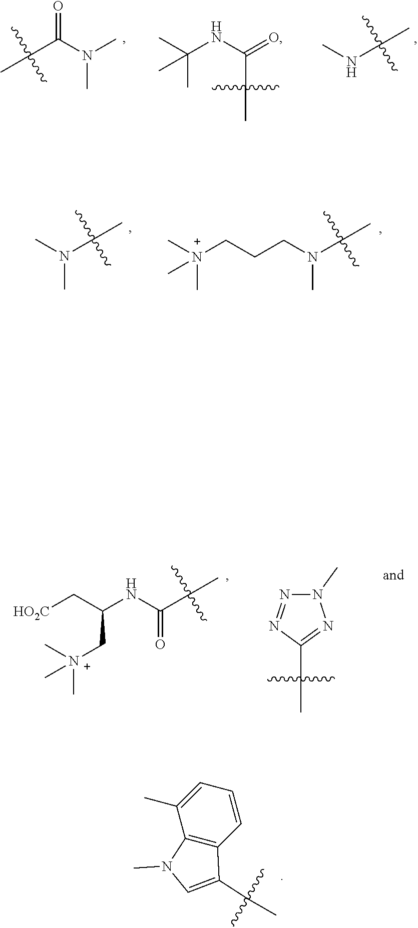

### Numbered Embodiment 173

173. The cyclic peptide of any one of the preceding embodiments, wherein the at least one amino acid residue(s) each independently comprising at least one masked amine and/or masked amide is/are independently selected from the group consisting of: NMe7MeW, AAMPhe, Paf(Ac), AEF(Ac), AcAEF, AEF(AcCh), AEF(Me)2, AEF(N(Me)2), AEF(MePrpa), AEF(NMe), Dab(NMeAc), Dab(NMecarn), Dab(NMeCOmPEG6), Dab(NMecPEG2a), Dab(NMecPEG3a), Dab(NMecPEG5a), Dab(NMecPEG5aCO)-, Dap(Ac), Dap(NMeAc), K(Ac), K(NMeAc), K(NMeCOmPEG6), K(NMeCOPEG4N+Me3), K(NMeCOPEG5a), K(NMecPEG5a), K(NMePEG5a), K(NMePEG3a), K(NmPEG6Ac), NMe3Pya, NMebAla, NMeDTyr, N(N(Me)), N(NMe), N(N(Me)2), Q(N(Me)2), Q(NHtBu) and Tetrazole(NMe).

### Numbered Embodiment 174

174. The cyclic peptide of any one of the preceding embodiments, wherein when the amino acid residue at position Z³ is replaced with an amino acid residue that is different from the amino acid residue recited at said position, the amino acid residue at position Z³ is replaced with an amino acid residue selected from the group consisting of: APEG2ser, APEG2Ser, APEG2Ser(S*), e(c), e(C), hk(Me)₃, k(5cpa), k(cPEG3a), k(d), k(D), k(dPEG12Ac), k(dPEG6Ac), k(dPEG9Ac), k(Me)₃, K(Me)₃, k(PEG2PEG2gEC12), k(PEG2PEG2gEC14), k(PEG2PEG2PEG2PEG2gEC12), k(PEG2PEG6gEC12), SP6, APEG2Ser(RS), gPEG2Ser and k(PEG2PEG2gE(c)C₁₂.

### Numbered Embodiment 175

175. The cyclic peptide of any one of the preceding embodiments, wherein when the amino acid residue at position Z³ is replaced with an amino acid residue that is different from the amino acid residue recited at said position, the amino acid residue at position Z³ is replaced with an amino acid residue comprising at least one quaternary amine selected from the group consisting of: APEG2ser, APEG2Ser, e(c), e(C), hk(Me)₃, k(5cpa), k(cPEG3a), k(d), k(D), K(Me)₃ and k(Me)₃.

### Numbered Embodiment 176

176. The cyclic peptide of any one of the preceding embodiments, wherein when the amino acid residue at position Z³ is replaced with an amino acid residue that is different from the amino acid residue recited at said position, the amino acid residue at position Z³ is replaced with an amino acid residue that is r substituted on the side chain with one or more groups selected from: —OH, —(C₁-C₄) alkyl, —O(C₁-C₄) alkyl, and —CN.

### Numbered Embodiment 177

177. The cyclic peptide of any one of the preceding embodiments, wherein when the amino acid residue at position Z³ is replaced with an amino acid residue that is different from the amino acid residue recited at said position, the amino acid residue at position Z³ is replaced with a hydrophilic amino acid residue.

### Numbered Embodiment 178

178. The cyclic peptide of any one of the preceding embodiments, wherein when the amino acid residue at position Z³ is replaced with an amino acid residue that is different from the amino acid residue recited at said position, the amino acid residue at position Z³ is replaced with a basic amino acid residue.

### Numbered Embodiment 179

179. The cyclic peptide of any one of the preceding embodiments, wherein when the amino acid residue at position Z³ is replaced with an amino acid residue that is different from the amino acid residue recited at said position, the amino acid residue at position Z³ is replaced with an acidic amino acid residue.

### Numbered Embodiment 180

180. The cyclic peptide of any one of the preceding embodiments, wherein the amino acid residue at position Z³ is replaced with an amino acid residue that is different from the amino acid residue recited at said position.

### Numbered Embodiment 181

181. The cyclic peptide of any one of the preceding embodiments, wherein the amino acid residue at position Z⁴ is the residue of Abu or the residue of an amino acid comprising a sulfhydryl group selected from the group consisting of: Pen, C or aMeC.

### Numbered Embodiment 182

182. The cyclic peptide of any one of the preceding embodiments, wherein the amino acid residue at position Z⁴ is the residue of Pen.

### Numbered Embodiment 183

183. The cyclic peptide of any one of the preceding embodiments, wherein when the amino acid residue at position Z⁵ is replaced with an amino acid residue that is different from the amino acid residue recited at said position, the amino acid residue at position Z⁵ is replaced with an amino acid residue selected from the group consisting of: A, APEG2Ser(S*), Dab(Me)₃, F, Gab, K(cPEG3a), K(Me)₃, K(PEG2PEG2gEC12), K(PEG2PEG2gEC14), L, N, N(N(Me)), N(NMe), Q and W.

### Numbered Embodiment 184

184. The cyclic peptide of any one of the preceding embodiments, wherein when the amino acid residue at position Z⁵ is replaced with an amino acid residue that is different from the amino acid residue recited at said position, the amino acid residue at position Z⁵ is replaced with an amino acid residue comprising at least one quaternary amine selected from the group consisting of: APEG2Ser(S*), Dab(Me)₃, K(cPEG3a) and K(Me)₃.

### Numbered Embodiment 185

185. The cyclic peptide of any one of the preceding embodiments, wherein when the amino acid residue at position Z⁵ is replaced with an amino acid residue that is different from the amino acid residue recited at said position, the amino acid residue at position Z⁵ is replaced with an amino acid residue that is N(N(Me)₂) substituted on the side chain with one or more groups selected from: —OH, —(C₁-C₄) alkyl, —O(C₁-C₄) alkyl, and —CN.

### Numbered Embodiment 186

186. The cyclic peptide of any one of the preceding embodiments, wherein when the amino acid residue at position Z⁵ is replaced with an amino acid residue that is different from the amino acid residue recited at said position, the amino acid residue at position Z⁵ is replaced with an aromatic amino acid residue.

### Numbered Embodiment 187

187. The cyclic peptide of any one of the preceding embodiments, wherein when the amino acid residue at position Z⁵ is replaced with an amino acid residue that is different from the amino acid residue recited at said position, the amino acid residue at position Z⁵ is replaced with an aliphatic amino acid residue.

### Numbered Embodiment 188

188. The cyclic peptide of any one of the preceding embodiments, wherein when the amino acid residue at position Z⁵ is replaced with an amino acid residue that is different from the amino acid residue recited at said position, the amino acid residue at position Z⁵ is replaced with a hydrophilic amino acid residue.

### Numbered Embodiment 189

189. The cyclic peptide of any one of the preceding embodiments, wherein when the amino acid residue at position Z⁵ is replaced with an amino acid residue that is different from the amino acid residue recited at said position, the amino acid residue at position Z⁵ is replaced with a basic amino acid residue.

### Numbered Embodiment 190

190. The cyclic peptide of any one of the preceding embodiments, wherein when the amino acid residue at position Z⁵ is replaced with an amino acid residue that is different from the amino acid residue recited at said position, the amino acid residue at position Z⁵ is replaced with an acidic amino acid residue.

### Numbered Embodiment 191

191. The cyclic peptide of any one of the preceding embodiments, wherein the amino acid residue at position Z⁵ is replaced with an amino acid residue that is different from the amino acid residue recited at said position.

### Numbered Embodiment 192

192. The cyclic peptide of any one of the preceding embodiments, wherein when the amino acid residue at position Z⁶ is replaced with an amino acid residue that is different from the amino acid residue recited at said position, the amino acid residue at position Z⁶ is replaced with an amino acid residue selected from the group consisting of: A and L.

### Numbered Embodiment 193

193. The cyclic peptide of any one of the preceding embodiments, wherein when the amino acid residue at position Z⁶ is replaced with an amino acid residue that is different from the amino acid residue recited at said position, the amino acid residue at position Z⁶ is replaced with an amino acid residue that is T substituted on the side chain with one or more groups selected from: —OH, —(C₁-C₄) alkyl, —O(C₁-C₄) alkyl, and —CN.

### Numbered Embodiment 194

194. The cyclic peptide of any one of the preceding embodiments, wherein when the amino acid residue at position Z⁶ is replaced with an amino acid residue that is different from the amino acid residue recited at said position, the amino acid residue at position Z⁶ is replaced with an aliphatic amino acid residue.

### Numbered Embodiment 195

195. The cyclic peptide of any one of the preceding embodiments, wherein when the amino acid residue at position Z⁶ is replaced with an amino acid residue that is different from the amino acid residue recited at said position, the amino acid residue at position Z⁶ is replaced with a polar amino acid residue.

### Numbered Embodiment 196

196. The cyclic peptide of any one of the preceding embodiments, wherein the amino acid residue at position Z⁶ is replaced with an amino acid residue that is different from the amino acid residue recited at said position.

### Numbered Embodiment 197

197. The cyclic peptide of any one of the preceding embodiments, wherein when the amino acid residue at position Z⁷ is replaced with an amino acid residue that is different from the amino acid residue recited at said position, the amino acid residue at position Z⁷ is replaced with an amino acid residue selected from the group consisting of: W, 7(3NAcPh)W, 7CF3W, Nme7MeW, 2Nal, A, F and L.

### Numbered Embodiment 198

198. The cyclic peptide of any one of the preceding embodiments, wherein when the amino acid residue at position Z⁷ is replaced with an amino acid residue that is different from the amino acid residue recited at said position, the amino acid residue at position Z⁷ is replaced with an amino acid residue that is 7MeW substituted on the side chain with one or more groups selected from: —OH, —(C₁-C₄) alkyl, —O(C₁-C₄) alkyl, and —CN.

### Numbered Embodiment 199

199. The cyclic peptide of any one of the preceding embodiments, wherein when the amino acid residue at position Z⁷ is replaced with an amino acid residue that is different from the amino acid residue recited at said position, the amino acid residue at position Z⁷ is replaced with an aromatic amino acid residue.

### Numbered Embodiment 200

200. The cyclic peptide of any one of the preceding embodiments, wherein when the amino acid residue at position Z⁷ is replaced with an amino acid residue that is different from the amino acid residue recited at said position, the amino acid residue at position Z⁷ is replaced with an aliphatic amino acid residue.

### Numbered Embodiment 201

201. The cyclic peptide of any one of the preceding embodiments, wherein the amino acid residue at position Z⁷ is replaced with an amino acid residue that is different from the amino acid residue recited at said position.

### Numbered Embodiment 202

202. The cyclic peptide of any one of the preceding embodiments, wherein when the amino acid residue at position Z⁸ is replaced with an amino acid residue that is different from the amino acid residue recited at said position, the amino acid residue at position Z⁸ is replaced with an amino acid residue selected from the group consisting of. K(NMeAC), Q, 4AmPhe, A, AIB, APEG2Ser, APEG2Ser(R*), APEG2Ser(S*), Cit, Dab(NMeAc), Dab(NMecarn), Dab(NMeCarn), Dab(NMeCOmPEG6), Dab(NMecPEG2a), Dab(NMecPEG3a), Dab(NMecPEG5a), Dap(NMeAc), F, K(4cpg), K(Ac), K(cPEG3a), K(Me)₃, K(NMeCOmPEG6), K(NMePEG3a), K(NmPEG6Ac), K(PEG2PEG2gEC12), K(PEG2PEG2gEC14), L, Paf(Ac), Q(N(Me)2), Q(NHtBu), W, Y and K(cPEG3aCO).

### Numbered Embodiment 203

203. The cyclic peptide of any one of the preceding embodiments, wherein when the amino acid residue at position Z⁸ is replaced with an amino acid residue that is different from the amino acid residue recited at said position, the amino acid residue at position Z⁸ is replaced with an amino acid residue comprising at least one quaternary amine selected from the group consisting of. APEG2Ser, APEG2ser, APEG2Ser(R*), APEG2Ser(S*), Dab(NMecarn), Dab(NmeCarn), Dab(NMecPEG2a), Dab(NMecPEG3a), Dab(NMecPEG5a), K(cPEG3a), K(Me)₃ and K(NMePEG3a).

### Numbered Embodiment 204

204. The cyclic peptide of any one of the preceding embodiments, wherein when the amino acid residue at position Z⁸ is replaced with an amino acid residue that is different from the amino acid residue recited at said position, the amino acid residue at position Z⁸ is replaced with an amino acid residue that is K(NMeAc) substituted on the side chain with one or more groups selected from: —OH, —(C₁-C₄) alkyl, —O(C₁-C₄) alkyl, and —CN.

### Numbered Embodiment 205

205. The cyclic peptide of any one of the preceding embodiments, wherein when the amino acid residue at position Z⁸ is replaced with an amino acid residue that is different from the amino acid residue recited at said position, the amino acid residue at position Z⁸ is replaced with an aromatic amino acid residue.

### Numbered Embodiment 206

206. The cyclic peptide of any one of the preceding embodiments, wherein when the amino acid residue at position Z⁸ is replaced with an amino acid residue that is different from the amino acid residue recited at said position, the amino acid residue at position Z⁸ is replaced with an aliphatic amino acid residue.

### Numbered Embodiment 207

207. The cyclic peptide of any one of the preceding embodiments, wherein when the amino acid residue at position Z⁸ is replaced with an amino acid residue that is different from the amino acid residue recited at said position, the amino acid residue at position Z⁸ is replaced with a hydrophilic amino acid residue.

### Numbered Embodiment 208

208. The cyclic peptide of any one of the preceding embodiments, wherein when the amino acid residue at position Z⁸ is replaced with an amino acid residue that is different from the amino acid residue recited at said position, the amino acid residue at position Z⁸ is replaced with a basic amino acid residue.

### Numbered Embodiment 209

209. The cyclic peptide of any one of the preceding embodiments, wherein when the amino acid residue at position Z⁸ is replaced with an amino acid residue that is different from the amino acid residue recited at said position, the amino acid residue at position Z⁸ is replaced with an acidic amino acid residue.

### Numbered Embodiment 210

210. The cyclic peptide of any one of the preceding embodiments, wherein the amino acid residue at position Z⁸ is replaced with an amino acid residue that is different from the amino acid residue recited at said position.

### Numbered Embodiment 211

211. The cyclic peptide of any one of the preceding embodiments, wherein the amino acid residue at position Z⁹ is the residue of an amino acid comprising a sulfhydryl group selected from the group consisting of: Pen, C or aMeC.

### Numbered Embodiment 212

212. The cyclic peptide of any one of the preceding embodiments, wherein the amino acid residue at position Z⁹ is the residue of Pen.

### Numbered Embodiment 213

213. The cyclic peptide of any one of the preceding embodiments, wherein when the amino acid residue at position Z¹⁰ is replaced with an amino acid residue that is different from the amino acid residue recited at said position, the amino acid residue at position Z¹⁰ is replaced with an amino acid residue selected from the group consisting of: AEF, 4DMPEF, 4DMPzEF, 4TMABYF, ACHMF, AEF((Ch)cPEG3a), AEF(Ac), AEF(AcCh), AEF(aPEG2a), AEF(BisMEP), AEF(BisMEPa), AEF(BisPEG2a)(RS), AEF(BisPEG2a)(S*), AEF(G), AEF(Me)2, AEF(MEP), AEF(MePrpa), AEF(N(Me)2), AEF(NHCh), AEF(NHcPEG3a), AEF(NMe), AEF(NMe2mPEG3), AEF(NMe3), AEF(NMeBismPEG3), AEF(NMePEG2a), AEF(NmPEG6), AEF(NsCh), AEF(PEG2a), AEF(SPD), APEG2F, APEG3F, dFPPEG3F, Diazabiclyclooctane6F, DMMMF, DMPMF, DMTASF, F(4G), F(4N3), F(4TzlDMA4mPEG), F(4TzlMME), F(4TzlMMo1), F(4TzlMMo3), F(4TzlMMo4), F(4TzlTMA1), F(4TzlTMA2), F(4TzlTMA3), F(4TzlTMA4), F(4TzlTMA5), GPEG3F, hFTMAPF, MMoEF, MMoPF, MMPEG3F, morfTMA4F, mPEG2TMA2F, mPEG2TMA4F, mPEG3TMA4F, MPzPEG3F, MTASF, NPyEF, NPyPEG3F, PiperazinequatF, TBAPEG3F, TMA3F, TMA4F, TMA6F, TMA8F, Tzl(Ch), TzlChmPEG, TzlChmPEG3, Y(C₉OH), Y(OEOXIMECh), Y(OTzlCh), Y(OTzlChC16), Y(OTzlChC8), Y(OTzlC1aC8), Y(OTzlChmPEG), Y(OTzlChmPEG3), Y(OTzlPEG3a), Y(OTzlPEG4a), Y(OTzlTMA4), Y(OZOXIMECh), YC8CO(NHPEG3a), YC8COPip, YCF2H, ACHMF(R*, S*), ACHMF(S*, S*), AEF(cPEG3a), APF, F(4TzlAme2), F(4TzlG2), F(4TzlMMo7) and F(4TzlTMA7).

### Numbered Embodiment 214

214. The cyclic peptide of any one of the preceding embodiments, wherein when the amino acid residue at position Z¹⁰ is replaced with an amino acid residue that is different from the amino acid residue recited at said position, the amino acid residue at position Z¹⁰ is replaced with an amino acid residue comprising at least one quaternary amine selected from the group consisting of: 4DMPEF, 4DMPzEF, 4TMABYF, ACHMF, ACHMF(R*, S*), ACHMF(S*, S*), AEF((Ch)cPEG3a), AEF(AcCh), AEF(aPEG2a), AEF(cPEG3a), AEF(BisMEPa), AEF(BisPEG2a)(RS), AEF(BisPEG2a)(S*), AEF(MePrpa), AEF(NHcPEG3a), AEF(NMe2mPEG3), AEF(NMe3), AEF(NMeBismPEG3), AEF(NMePEG2a), AEF(NsCh), AEF(NHCh), AEF(PEG2a), AEF(SPD), APEG2F, APEG3F, dFPPEG3F, Diazabiclyclooctane6F, DMMMF, DMPMF, DMTASF, F(4TzlDMA4mPEG), F(4TzlMMo1), F(4TzlMMo3), F(4TzlMMo4), F(4TzlTMA1), F(4TzlTMA2), F(4TzlTMA3), F(4TzlTMA4), F(4TzlTMA5), F(4TzlMMo7), F(4TzlTMA7), hFTMAPF, MMoEF, MMoPF, MMPEG3F, morfTMA4F, mPEG2TMA2F, mPEG2TMA4F, mPEG3TMA4F, MPzPEG3F, MTASF, NPyEF, NPyPEG3F, PiperazinequatF, TBAPEG3F, TMA3F, TMA4F, TMA6F, TMA8F, Tzl(Ch), TzlChmPEG, TzlChmPEG3, Y(OEOXIMECh), Y(OTzlCh), Y(OTzlChC16), Y(OTzlChC8), Y(OTzlC1aC8), Y(OTzlChmPEG), Y(OTzlChmPEG3), Y(OTzlPEG3a), Y(OTzlPEG4a), Y(OTzlTMA4), Y(OZOXIMECh) and YC8CO(NHPEG3a).

### Numbered Embodiment 215

215. The cyclic peptide of any one of the preceding embodiments, wherein when the amino acid residue at position Z¹⁰ is replaced with an amino acid residue that is different from the amino acid residue recited at said position, the amino acid residue at position Z¹⁰ is replaced with an amino acid residue that is TMAPF substituted on the side chain with one or more groups selected from: —OH, —(C₁-C₄) alkyl, —O(C₁-C₄) alkyl, and —CN.

### Numbered Embodiment 216

216. The cyclic peptide of any one of the preceding embodiments, wherein when the amino acid residue at position Z¹⁰ is replaced with an amino acid residue that is different from the amino acid residue recited at said position, the amino acid residue at position Z¹⁰ is replaced with an amino acid residue having the following structure:

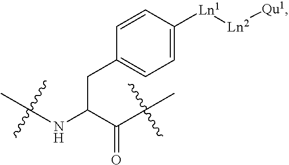

wherein
Ln¹ is selected from the group consisting of: an —O—atom,

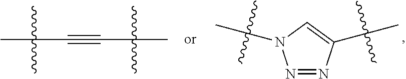

wherein the right hand side of each depicted structure is bound to Ln²;
Ln² is a linker moiety comprising
   (i) at least 4 atoms, for example an optionally substituted alkylene comprising at least C₄ atoms, an optionally substituted heteroalkylene comprising at least C₄ atoms, an optionally substituted heteroalkenylene comprising at least C₄ atoms, an optionally substituted alkynylene comprising at least C₄ atoms, an optionally substituted alkylene—optionally substituted carbocyclyl comprising at least C₄ atoms, or an optionally substituted alkylene—optionally substituted heterocyclyl comprising at least C₄ atoms, for example optionally substituted (C₄-C₂₀) alkylene, optionally substituted (C₄-C₂₀) heteroalkylene, optionally substituted (C₄-C₂₀) heteroalkenylene, optionally substituted (C₄-C₂₀) alkynylene, optionally substituted (C₁-C₃) alkylene —optionally substituted (CH₃—C₁₀) carbocyclyl, or optionally substituted (C₁-C₃) alkylene—optionally substituted (CH₃—C₁₄) heterocyclyl, or
   (ii) at least 2 atoms when Ln is

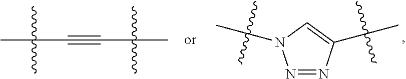

for example an optionally substituted alkylene comprising at least C₂ atoms or an optionally substituted heteroalkylene comprising at least C₂ atoms, for example optionally substituted (C₂-C₂₀) alkylene or optionally substituted (C₂-C₂₀) heteroalkylene; and
Qu¹ is a quaternary amine.

### Numbered Embodiment 217

217. The cyclic peptide of any one of the preceding embodiments, wherein when the amino acid residue at position Z¹⁰ is replaced with an amino acid residue that is different from the amino acid residue recited at said position, the amino acid residue at position Z¹⁰ is replaced with an amino acid residue having the following structure:

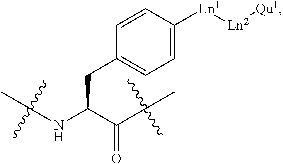

wherein Ln¹, Ln² and Qu^{l} are as defined in any one of the preceding embodiments.

### Numbered Embodiment 218

218. The cyclic peptide of any one of the preceding embodiments, wherein Qu¹ is a quaternary amine as defined in any one of the preceding embodiments.

### Numbered Embodiment 219

219. The cyclic peptide of any one of the preceding embodiments, wherein Ln¹ is an —O-atom.

### Numbered Embodiment 220

220. The cyclic peptide of any one of the preceding embodiments, wherein Ln² is selected from the group consisting of: optionally substituted (C₄-C₁₅) alkylene, optionally substituted (C₄-C₁₅) heteroalkylene, optionally substituted (C₄-C₁₅) heteroalkenylene, optionally substituted (C₄-C₁₅) alkynylene, optionally substituted (C₁-C₃) alkylene—optionally substituted (C₅-C₇) carbocyclyl, or optionally substituted (C₁-C₃) alkylene—optionally substituted (C₅-C₇) heterocyclyl.

### Numbered Embodiment 221

221. The cyclic peptide of any one of the preceding embodiments, wherein Ln² is selected from the group consisting of: optionally substituted (C₄-C₁₀) alkylene, optionally substituted (C₄-C₁₀) heteroalkylene, optionally substituted (C₄-C₁₀) heteroalkenylene, optionally substituted (C₄-C₁₀) alkynylene, optionally substituted (C₁-C₃) alkylene—optionally substituted (C) carbocyclyl, or optionally substituted (C₁-C₃) alkylene—optionally substituted (C) heterocyclyl.

### Numbered Embodiment 222

222. The cyclic peptide of any one of the preceding embodiments, wherein Ln² is selected from the group consisting of: optionally substituted (C₄-C₈) alkylene, optionally substituted (C₄-C₈) heteroalkylene, optionally substituted (C₄-C₈) heteroalkenylene, optionally substituted (C₄-C₈) alkynylene, optionally substituted (C₁-C₃) alkylene—optionally substituted (C₆) carbocyclyl, or optionally substituted (C₁-C₃) alkylene—optionally substituted (C₆) heterocyclyl.

### Numbered Embodiment 223

223. The cyclic peptide of any one of the preceding embodiments, wherein when Ln¹ is

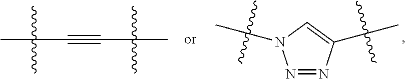

Ln² is selected from the group consisting of optionally substituted (C₂-C₁₅) alkylene or optionally substituted (C₂-C₁₅) heteroalkylene.

### Numbered Embodiment 224

224. The cyclic peptide of any one of the preceding embodiments, wherein when Ln¹ is

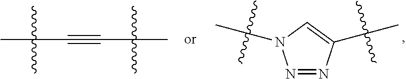

Ln² is selected from the group consisting of optionally substituted (C₂-C₁₀) alkylene or optionally substituted (C₂-C₁₀) heteroalkylene.

### Numbered Embodiment 225

225. The cyclic peptide of any one of the preceding embodiments, wherein when Ln¹ is

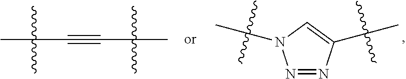

Ln² is selected from the group consisting of optionally substituted (C₂-C₅) alkylene or optionally substituted (C₂-C₅) heteroalkylene.

### Numbered Embodiment 226

226. The cyclic peptide of any one of the preceding embodiments, wherein when Ln² is optionally substituted alkylene, the optionally substituted alkylene is unsubstituted or is substituted with one or more substituents independently selected from the group consisting of Fluoro.

### Numbered Embodiment 227

227. The cyclic peptide of any one of the preceding embodiments, wherein when Ln² is optionally substituted heteroalkylene, the optionally substituted heteroalkylene is unsubstituted or is substituted with one or more substituents independently selected from the group consisting of: (C₁-C₃) alkyl,

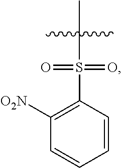 [SMILES: *S(=O)(=O)c1ccccc1[N+](=O)[O-]]

═O (i.e., two geminal hydrogens on a carbon atom of the heteroalkylene are replaced with the group ═O), or a quaternary amine of any one of the preceding embodiments, for example, —N⁺(CH₃)₃.

### Numbered Embodiment 228

228. The cyclic peptide of any one of the preceding embodiments, wherein when Ln² is optionally substituted heteroalkenylene, the optionally substituted heteroalkenylene is unsubstituted.

### Numbered Embodiment 229

229. The cyclic peptide of any one of the preceding embodiments, wherein when Ln² is optionally substituted alkynylene, the optionally substituted alkynylene is unsubstituted.

### Numbered Embodiment 230

230. The cyclic peptide of any one of the preceding embodiments, wherein when Ln² is optionally substituted alkylene—optionally substituted carbocyclyl, the optionally substituted alkylene—optionally substituted carbocyclyl is unsubstituted.

### Numbered Embodiment 231

231. The cyclic peptide of any one of the preceding embodiments, wherein when Ln² is optionally substituted alkylene—optionally substituted heterocyclyl, the optionally substituted alkylene—optionally substituted heterocyclyl is unsubstituted.

### Numbered Embodiment 232

232. The cyclic peptide of any one of the preceding embodiments, wherein when the amino acid residue at position Z¹⁰ is replaced with an amino acid residue that is different from the amino acid residue recited at said position, the amino acid residue at position Z¹⁰ is replaced with an amino acid residue having the following structure:

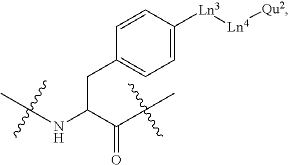

wherein
Ln³ is selected from the group consisting of: an —O—atom,

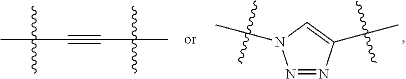

wherein the right hand side of each depicted structure is bound to Ln⁴;
Ln⁴ is a linker moiety comprising at least 2 atoms, for example an optionally substituted alkylene comprising at least C₂ atoms, an optionally substituted heteroalkylene comprising at least C₂ atoms, or an optionally substituted alkynylene comprising at least C₂ atoms, for example optionally substituted (C₂-C₂₀) alkylene, optionally substituted (C₂-C₂₀) heteroalkylene or optionally substituted (C₂-C₂₀) alkynylene; and
Qu² is a (CH₃—C₁₄) heterocyclyl quaternary amine, a (C₅-C₁₄) heteroaryl quaternary amine or a (C₅-C₁₄) bicyclic heterocyclyl quaternary amine, for example, a (CH₃—C₁₄) heterocyclyl quaternary amine selected from the group consisting of:

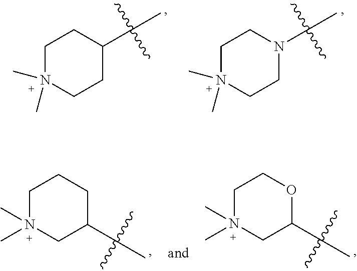

a (C₅-C₁₄) heteroaryl quaternary amine selected from the group consisting of:

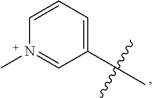

or a (C₅-C₁₄) bicyclic heterocyclyl quaternary amine selected from the group consisting of

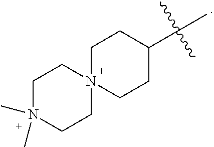

### Numbered Embodiment 233

233. The cyclic peptide of any one of the preceding embodiments, wherein when the amino acid residue at position Z¹⁰ is replaced with an amino acid residue that is different from the amino acid residue recited at said position, the amino acid residue at position Z¹⁰ is replaced with an amino acid residue having the following structure:

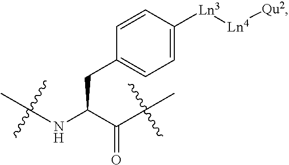

wherein Ln³, Ln⁴ and Qu² are as defined in any one of the preceding embodiments.

### Numbered Embodiment 234

234. The cyclic peptide of any one of the preceding embodiments, wherein Qu² is a quaternary amine as defined in any one of the preceding embodiments.

### Numbered Embodiment 235

235. The cyclic peptide of any one of the preceding embodiments, wherein Ln³ is an —O-atom.

### Numbered Embodiment 236

236. The cyclic peptide of any one of the preceding embodiments, wherein Ln⁴ is selected from the group consisting of: optionally substituted (C₂-C₁₅) alkylene, optionally substituted (C₂-C₁₅) heteroalkylene or optionally substituted (C₂-C₁₅) alkynylene.

### Numbered Embodiment 237

237. The cyclic peptide of any one of the preceding embodiments, wherein Ln⁴ is selected from the group consisting of: optionally substituted (C₂-C₁₀) alkylene, optionally substituted (C₂-C₁₀) heteroalkylene or optionally substituted (C₂-C₁₀) alkynylene.

### Numbered Embodiment 238

238. The cyclic peptide of any one of the preceding embodiments, wherein Ln⁴ is selected from the group consisting of: optionally substituted (C₂-C₈) alkylene, optionally substituted (C₂-C₈) heteroalkylene or optionally substituted (C₂-C₈) alkynylene.

### Numbered Embodiment 239

239. The cyclic peptide of any one of the preceding embodiments, wherein when Ln⁴ is optionally substituted alkylene, the optionally substituted alkylene is unsubstituted or is substituted with one or more substituents independently selected from the group consisting of Fluoro.

### Numbered Embodiment 240

240. The cyclic peptide of any one of the preceding embodiments, wherein when Ln⁴ is optionally substituted heteroalkylene, the optionally substituted heteroalkylene is unsubstituted or is substituted with one or more substituents independently selected from the group consisting of: (C₁-C₃) alkyl,

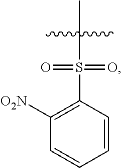 [SMILES: *S(=O)(=O)c1ccccc1[N+](=O)[O-]]

═O (i.e., two geminal hydrogens on a carbon atom of the heteroalkylene are replaced with the group ═O), or a quaternary amine of any one of the preceding embodiments, for example, —N⁺(CH₃)₃.

### Numbered Embodiment 241

241. The cyclic peptide of any one of the preceding embodiments, wherein when Ln⁴ is optionally substituted alkynylene, the optionally substituted alkynylene is unsubstituted.

### Numbered Embodiment 242

242. The cyclic peptide of any one of the preceding embodiments, wherein when the amino acid residue at position Z¹⁰ is replaced with an amino acid residue that is different from the amino acid residue recited at said position, the amino acid residue at position Z¹⁰ is replaced with an aromatic amino acid residue.

### Numbered Embodiment 243

243. The cyclic peptide of any one of the preceding embodiments, wherein the amino acid residue at position Z¹⁰ is replaced with an amino acid residue that is different from the amino acid residue recited at said position.

### Numbered Embodiment 244

244. The cyclic peptide of any one of the preceding embodiments, wherein when the amino acid residue at position Z¹¹ is replaced with an amino acid residue that is different from the amino acid residue recited at said position, the amino acid residue at position Z¹¹ is replaced with an amino acid residue selected from the group consisting of: A, F, L and W.

### Numbered Embodiment 245

245. The cyclic peptide of any one of the preceding embodiments, wherein when the amino acid residue at position Z¹¹ is replaced with an amino acid residue that is different from the amino acid residue recited at said position, the amino acid residue at position Z¹¹ is replaced with an amino acid residue that is 2Nal substituted on the side chain naphthalene ring with one or more groups selected from: —OH, —(C₁-C₄) alkyl, —O(C₁-C₄) alkyl, and —CN.

### Numbered Embodiment 246

246. The cyclic peptide of any one of the preceding embodiments, wherein when the amino acid residue at position Z¹¹ is replaced with an amino acid residue that is different from the amino acid residue recited at said position, the amino acid residue at position Z¹¹ is replaced with an aromatic amino acid residue.

### Numbered Embodiment 247

247. The cyclic peptide of any one of the preceding embodiments, wherein when the amino acid residue at position Z¹¹ is replaced with an amino acid residue that is different from the amino acid residue recited at said position, the amino acid residue at position Z¹¹ is replaced with an aliphatic amino acid residue.

### Numbered Embodiment 248

248. The cyclic peptide of any one of the preceding embodiments, wherein the amino acid residue at position Z¹¹ is replaced with an amino acid residue that is different from the amino acid residue recited at said position.

### Numbered Embodiment 249

249. The cyclic peptide of any one of the preceding embodiments, wherein when the amino acid residue at position Z¹² is replaced with an amino acid residue that is different from the amino acid residue recited at said position, the amino acid residue at position Z¹² is replaced with an amino acid residue selected from the group consisting of: A, Achx, Achx(diF), Acpx, Aib, AIB, aMeK, aMeL, Chg, diFCpx, F, L, Pip(NMe), Pip(NMe2), W and diFAchx.

### Numbered Embodiment 250

250. The cyclic peptide of any one of the preceding embodiments, wherein when the amino acid residue at position Z¹² is replaced with an amino acid residue that is different from the amino acid residue recited at said position, the amino acid residue at position Z¹² is replaced with an amino acid residue comprising at least one quaternary amine selected from the group consisting of: Pip(NMe2).

### Numbered Embodiment 251

251. The cyclic peptide of any one of the preceding embodiments, wherein when the amino acid residue at position Z¹² is replaced with an amino acid residue that is different from the amino acid residue recited at said position, the amino acid residue at position Z¹² is replaced with an amino acid residue that is THP substituted on the side chain with one or more groups selected from: —OH, —(C₁-C₄) alkyl, —O(C₁-C₄) alkyl, and —CN.

### Numbered Embodiment 252

252. The cyclic peptide of any one of the preceding embodiments, wherein when the amino acid residue at position Z¹² is replaced with an amino acid residue that is different from the amino acid residue recited at said position, the amino acid residue at position Z¹² is replaced with an aromatic amino acid residue.

### Numbered Embodiment 253

253. The cyclic peptide of any one of the preceding embodiments, wherein when the amino acid residue at position Z¹² is replaced with an amino acid residue that is different from the amino acid residue recited at said position, the amino acid residue at position Z¹² is replaced with an aliphatic amino acid residue.

### Numbered Embodiment 254

254. The cyclic peptide of any one of the preceding embodiments, wherein when the amino acid residue at position Z¹² is replaced with an amino acid residue that is different from the amino acid residue recited at said position, the amino acid residue at position Z¹² is replaced with a polar amino acid residue.

### Numbered Embodiment 255

255. The cyclic peptide of any one of the preceding embodiments, wherein when the amino acid residue at position Z¹² is replaced with an amino acid residue that is different from the amino acid residue recited at said position, the amino acid residue at position Z¹² is replaced with a hydrophilic amino acid residue.

### Numbered Embodiment 256

256. The cyclic peptide of any one of the preceding embodiments, wherein when the amino acid residue at position Z¹² is replaced with an amino acid residue that is different from the amino acid residue recited at said position, the amino acid residue at position Z¹² is replaced with a basic amino acid residue.

### Numbered Embodiment 257

257. The cyclic peptide of any one of the preceding embodiments, wherein the amino acid residue at position Z¹² is replaced with an amino acid residue that is different from the amino acid residue recited at said position.

### Numbered Embodiment 258

258. The cyclic peptide of any one of the preceding embodiments, wherein when the amino acid residue at position Z¹³ is replaced with an amino acid residue that is different from the amino acid residue recited at said position, the amino acid residue at position Z¹³ is replaced with an amino acid residue selected from the group consisting of: K(NMeAC), E, A, AIB, aMeE, APEG2Ser, APEG2Ser(S*), Cit, Dab(NMeAc), Dab(NMecarn), Dab(NMeCOmPEG6), Dab(NMecPEG2a), Dab(NMecPEG3a), Dap(Ac), Dap(NMeAc), E(c), E(C), F, K(5cpa), K(Ac), K(cPEG3a), K(d), K(D), K(dPEG12Ac), K(dPEG6Ac), K(dPEG9Ac), K(Me)₃, K(NMeCOmPEG6), K(NMeCOPEG4N+Me3), K(NMePEG3a), K(NmPEG6Ac), K(PEG2PEG2gEC12), K(PEG2PEG2gEC14), K(PEG2PEG2PEG2gEC12), L, Q(N(Me)2), Tetrazole, Tetrazole(NMe), W, K(DFN), K(IPB) and Ne.

### Numbered Embodiment 259

259. The cyclic peptide of any one of the preceding embodiments, wherein when the amino acid residue at position Z¹³ is replaced with an amino acid residue that is different from the amino acid residue recited at said position, the amino acid residue at position Z¹³ is replaced with an amino acid residue comprising at least one quaternary amine selected from the group consisting of: APEG2Ser, APEG2Ser(S*), Dab(NMecarn), Dab(NMecPEG2a), Dab(NMecPEG3a), K(5cpa), K(cPEG3a), E(c), E(C), K(d), K(D), K(Me)₃, K(NMeCOPEG4N+Me3) and K(NMePEG3a).

### Numbered Embodiment 260

260. The cyclic peptide of any one of the preceding embodiments, wherein when the amino acid residue at position Z¹³ is replaced with an amino acid residue that is different from the amino acid residue recited at said position, the amino acid residue at position Z¹³ is replaced with an amino acid residue that is K(NMeAc) substituted on the side chain with one or more groups selected from: —OH, —(C₁-C₄) alkyl, —O(C₁-C₄) alkyl, and —CN.

### Numbered Embodiment 261

261. The cyclic peptide of any one of the preceding embodiments, wherein when the amino acid residue at position Z¹³ is replaced with an amino acid residue that is different from the amino acid residue recited at said position, the amino acid residue at position Z¹³ is replaced with an aromatic amino acid residue.

### Numbered Embodiment 262

262. The cyclic peptide of any one of the preceding embodiments, wherein when the amino acid residue at position Z¹³ is replaced with an amino acid residue that is different from the amino acid residue recited at said position, the amino acid residue at position Z¹³ is replaced with an aliphatic amino acid residue.

### Numbered Embodiment 263

263. The cyclic peptide of any one of the preceding embodiments, wherein when the amino acid residue at position Z¹³ is replaced with an amino acid residue that is different from the amino acid residue recited at said position, the amino acid residue at position Z¹³ is replaced with a hydrophilic amino acid residue.

### Numbered Embodiment 264

264. The cyclic peptide of any one of the preceding embodiments, wherein when the amino acid residue at position Z¹³ is replaced with an amino acid residue that is different from the amino acid residue recited at said position, the amino acid residue at position Z¹³ is replaced with an acidic amino acid residue.

### Numbered Embodiment 265

265. The cyclic peptide of any one of the preceding embodiments, wherein the amino acid residue at position Z¹³ is replaced with an amino acid residue that is different from the amino acid residue recited at said position.

### Numbered Embodiment 266

266. The cyclic peptide of any one of the preceding embodiments, wherein when the amino acid residue at position Z¹⁴ is replaced with an amino acid residue that is different from the amino acid residue recited at said position, the amino acid residue at position Z¹⁴ is replaced with an amino acid residue selected from the group consisting of: K(Ac) and N(NMe).

### Numbered Embodiment 267

267. The cyclic peptide of any one of the preceding embodiments, wherein when the amino acid residue at position Z¹⁴ is replaced with an amino acid residue that is different from the amino acid residue recited at said position, the amino acid residue at position Z¹⁴ is replaced with an amino acid residue that is N substituted on the side chain with one or more groups selected from: —OH, —(C₁-C₄) alkyl, —O(C₁-C₄) alkyl, and —CN.

### Numbered Embodiment 268

268. The cyclic peptide of any one of the preceding embodiments, wherein when the amino acid residue at position Z¹⁴ is replaced with an amino acid residue that is different from the amino acid residue recited at said position, the amino acid residue at position Z¹⁴ is replaced with a hydrophilic amino acid residue.

### Numbered Embodiment 269

269. The cyclic peptide of any one of the preceding embodiments, wherein the amino acid residue at position Z¹⁴ is replaced with an amino acid residue that is different from the amino acid residue recited at said position.

### Numbered Embodiment 270

270. The cyclic peptide of any one of the preceding embodiments, wherein when the amino acid residue at position Z¹⁵ is replaced with an amino acid residue that is different from the amino acid residue recited at said position, the amino acid residue at position Z¹⁵ is replaced with an amino acid residue selected from the group consisting of: 3pya, 5CF33Pya, 5MePyridinAla, bAla, dK, dL, F, f, H, h, k, N, NMe3Pya, NMebAla, NMeDTyr, orn, Paf, s, t, THP, v, y and A.

### Numbered Embodiment 271

271. The cyclic peptide of any one of the preceding embodiments, wherein when the amino acid residue at position Z¹⁵ is replaced with an amino acid residue that is different from the amino acid residue recited at said position, the amino acid residue at position Z¹⁵ is replaced with an amino acid residue that is 3Pya substituted on the side chain with one or more groups selected from: —OH, —(C₁-C₄) alkyl, —O(C₁-C₄) alkyl, and —CN.

### Numbered Embodiment 272

272. The cyclic peptide of any one of the preceding embodiments, wherein when the amino acid residue at position Z¹⁵ is replaced with an amino acid residue that is different from the amino acid residue recited at said position, the amino acid residue at position Z¹⁵ is replaced with an amino acid residue comprising at least one quaternary amine selected from the group consisting of: NMe3Pya.

### Numbered Embodiment 273

273. The cyclic peptide of any one of the preceding embodiments, wherein when the amino acid residue at position Z¹⁵ is replaced with an amino acid residue that is different from the amino acid residue recited at said position, the amino acid residue at position Z¹⁵ is replaced with an aromatic amino acid residue.

### Numbered Embodiment 274

274. The cyclic peptide of any one of the preceding embodiments, wherein when the amino acid residue at position Z¹⁵ is replaced with an amino acid residue that is different from the amino acid residue recited at said position, the amino acid residue at position Z¹⁵ is replaced with an aliphatic amino acid residue.

### Numbered Embodiment 275

275. The cyclic peptide of any one of the preceding embodiments, wherein when the amino acid residue at position Z¹⁵ is replaced with an amino acid residue that is different from the amino acid residue recited at said position, the amino acid residue at position Z¹⁵ is replaced with a polar amino acid residue.

### Numbered Embodiment 276

276. The cyclic peptide of any one of the preceding embodiments, wherein when the amino acid residue at position Z¹⁵ is replaced with an amino acid residue that is different from the amino acid residue recited at said position, the amino acid residue at position Z¹⁵ is replaced with a hydrophilic amino acid residue.

### Numbered Embodiment 277

277. The cyclic peptide of any one of the preceding embodiments, wherein when the amino acid residue at position Z¹¹ is replaced with an amino acid residue that is different from the amino acid residue recited at said position, the amino acid residue at position Z¹¹ is replaced with a basic amino acid residue.

### Numbered Embodiment 278

278. The cyclic peptide of any one of the preceding embodiments, wherein when the amino acid residue at position Z¹¹ is replaced with an amino acid residue that is different from the amino acid residue recited at said position, the amino acid residue at position Z¹¹ is replaced with a non-polar amino acid residue.

### Numbered Embodiment 279

279. The cyclic peptide of any one of the preceding embodiments, wherein the amino acid residue at position Z¹¹ is replaced with an amino acid residue that is different from the amino acid residue recited at said position.

### Numbered Embodiment 280

280. The cyclic peptide of any one of the preceding embodiments, wherein when the amino acid residue at position Z¹⁶ is replaced with an amino acid residue that is different from the amino acid residue recited at said position, the amino acid residue at position Z¹⁶ is replaced with an amino acid residue selected from the group consisting of: 4diFPro, NMeDTyr, NMeK(PEG2PEG2C₁₂), NMeK(PEG2PEG2C₁₄), NMeK(PEG2PEG2gEC12), NMeK(PEG2PEG2gEC14), NMeK(PEG2PEG2K(PEG2PEG2gEC12)₂), NMeK(PEG2PEG2K(PEG2PEG2PEG2PEG2gEC12)₂), NMeK(PEG2PEG2PEG2gEC12), NMeK(PEG2PEG2PEG2PEG2gEC12), NMeK(PEG2PEG6gEC12), NMeK(PEG2PEG6gEC14), NMeK(SP6PEG2PEG2C₁₂) and NMeK(SP6PEG2PEG2gEC12).

### Numbered Embodiment 281

281. The cyclic peptide of any one of the preceding embodiments, wherein when the amino acid residue at position Z¹⁶ is replaced with an amino acid residue that is different from the amino acid residue recited at said position, the amino acid residue at position Z¹⁶ is replaced with an amino acid residue comprising at least one quaternary amine selected from the group consisting of: NMeK(SP6PEG2PEG2C₁₂) and NMeK(SP6PEG2PEG2gEC12).

### Numbered Embodiment 282

282. The cyclic peptide of any one of the preceding embodiments, wherein when the amino acid residue at position Z¹⁶ is replaced with an amino acid residue that is different from the amino acid residue recited at said position, the amino acid residue at position Z¹⁶ is replaced with an amino acid residue that is Sar substituted on the side chain with one or more groups selected from: —OH, —(C₁-C₄) alkyl, —O(C₁-C₄) alkyl, and —CN.

### Numbered Embodiment 283

283. The cyclic peptide of any one of the preceding embodiments, wherein when the amino acid residue at position Z¹⁶ is replaced with an amino acid residue that is different from the amino acid residue recited at said position, the amino acid residue at position Z¹⁶ is replaced with an aromatic amino acid residue.

### Numbered Embodiment 284

284. The cyclic peptide of any one of the preceding embodiments, wherein when the amino acid residue at position Z¹⁶ is replaced with an amino acid residue that is different from the amino acid residue recited at said position, the amino acid residue at position Z¹⁶ is replaced with an acidic amino acid residue.

### Numbered Embodiment 285

285. The cyclic peptide of any one of the preceding embodiments, wherein when the amino acid residue at position Z¹⁶ is replaced with an amino acid residue that is different from the amino acid residue recited at said position, the amino acid residue at position Z¹⁶ is replaced with a polar amino acid residue.

### Numbered Embodiment 286

286. The cyclic peptide of any one of the preceding embodiments, wherein when the amino acid residue at position Z¹⁶ is replaced with an amino acid residue that is different from the amino acid residue recited at said position, the amino acid residue at position Z¹⁶ is replaced with a hydrophilic amino acid residue.

### Numbered Embodiment 287

287. The cyclic peptide of any one of the preceding embodiments, wherein when the amino acid residue at position Z¹⁶ is replaced with an amino acid residue that is different from the amino acid residue recited at said position, the amino acid residue at position Z¹⁶ is replaced with a non-polar amino acid residue.

### Numbered Embodiment 288

288. The cyclic peptide of any one of the preceding embodiments, wherein the amino acid residue at position Z¹⁶ is replaced with an amino acid residue that is different from the amino acid residue recited at said position.

### Numbered Embodiment 289

289. The cyclic peptide of any one of the preceding embodiments, wherein the amino acid residue(s) specified at a given position are substituted on the side chain with one or more groups selected from: —OH, —(C₁-C₄) alkyl, —O(C₁-C₄) alkyl, and —CN.

### Numbered Embodiment 290

290. The cyclic peptide of any one of the preceding embodiments, wherein R^{NT} is selected from the group consisting of: —C(O)-optionally substituted (C₁-C₁₅) alkyl and —C(O)-optionally substituted (C₁-C₃₅) heteroalkyl.

### Numbered Embodiment 291

291. The cyclic peptide of any one of the preceding embodiments, wherein R^{NT} is selected from the group consisting of: —C(O)-optionally substituted (C₁-C₁₀) alkyl and —C(O)-optionally substituted (C₁-C₃₅) heteroalkyl.

### Numbered Embodiment 292

292. The cyclic peptide of any one of the preceding embodiments, wherein R^{NT} is selected from the group consisting of: —C(O)-optionally substituted (C₁-C₈) alkyl and —C(O)-optionally substituted (C₁-C₃₀) heteroalkyl.

### Numbered Embodiment 293

293. The cyclic peptide of any one of the preceding embodiments, wherein R^{NT} is selected from the group consisting of: —C(O)-optionally substituted (C₁-C₆) alkyl and —C(O)-optionally substituted (C₁-C₃₀) heteroalkyl.

### Numbered Embodiment 294

294. The cyclic peptide of any one of the preceding embodiments, wherein R^{NT} is selected from the group consisting of: (d)gEPEG2PEG2CO, 4cpgCO, 5cpaCO, AcdPEG12CO, AcdPEG6CO, AcdPEG9CO, CEPEG2PEG2CO, C₁₄gEPEG2PEG2CO, CF3CO, CF3propylamide, cPEG2gCO, cPEG3aCO, cPEG3gCO, cPEG5aCO, EtCO, F3CO, MeCO, mPEG3CO, mPEG6CO, cPEG3AmCO, DFNCO, DFNPEG2PEG2CO, IPBCO and IPBPEG2PEG2CO.

### Numbered Embodiment 295

295. The cyclic peptide of any one of the preceding embodiments, wherein R^{NT} is an —C(O)-optionally substituted (C₁-C₂₀) alkyl, for example, 4cpgCO, CF3CO, CF3propylamide, EtCO, F3CO, MeCO or 5cpaCO.

### Numbered Embodiment 296

296. The cyclic peptide of any one of the preceding embodiments, wherein R^{NT} is an —C(O)-optionally substituted (C₁-C₂₀) alkyl that comprises a quaternary amine, for example, 5cpaCO.

### Numbered Embodiment 297

297. The cyclic peptide of any one of the preceding embodiments, wherein R^{NT} is an —C(O)-optionally substituted (C₁-C₄₀) heteroalkyl, for example, AcdPEG12CO, AcdPEG6CO, AcdPEG9CO, CEPEG2PEG2CO, cPEG2gCO, cPEG3gCO, mPEG3CO, mPEG6CO, (d)gEPEG2PEG2CO, cPEG3aCO, and cPEG5aCO.

### Numbered Embodiment 298

298. The cyclic peptide of any one of the preceding embodiments, wherein R^{NT} is an —C(O)-optionally substituted (C₁-C₄₀) heteroalkyl that comprises a quaternary amine, for example, (d)gEPEG2PEG2CO, cPEG3aCO, cPEG5aCO.

### Numbered Embodiment 299

299. The cyclic peptide of any one of the preceding embodiments, wherein R^{NT} is an —C(O)-optionally substituted heteroalkyl that comprises a hydrophilic polymer, for example, polyethylene glycol (PEG).

### Numbered Embodiment 300

300. The cyclic peptide of any one of the preceding embodiments, wherein R^{NT} is an —C(O)-optionally substituted heteroalkyl that comprises the formula —[O—CH₂CH₂]n-, wherein n is an integer, for example wherein:
(i) n is an integer from about 1 to about 20;
(ii) n is an integer from about 1 to about 15;
(iii) n is an integer from about 1 to about 10; or
(iv) n is an integer from about 2 to about 12.

### Numbered Embodiment 301

301. The cyclic peptide of any one of the preceding embodiments, wherein when R^{NT} is a —C(O)-optionally substituted heteroalkyl, the —C(O)-optionally substituted heteroalkyl is unsubstituted or is substituted with one or more substituents independently selected from the group consisting of: —OH, —NH₂, —CO₂H, —CO₂CH₃, —NH(C═NH)NH₂, ═O (i.e., two geminal hydrogens on a carbon atom of the heteroalkyl are replaced with the group ═O),

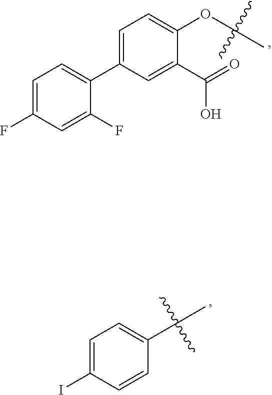 [SMILES: *Oc1ccc(-c2ccc(F)cc2F)cc1C(=O)O ; *c1ccc(I)cc1]

or a quaternary amine of any one of the preceding embodiments, for example, —N+(CH₃)₃.

### Numbered Embodiment 302

302. The cyclic peptide of any one of the preceding embodiments, wherein when R^{NT} is a —C(O)-optionally substituted alkyl, the —C(O)-optionally substituted alkyl is unsubstituted or is substituted with one or more substituents independently selected from the group consisting of: Fluoro, —NH(C═NH)NH₂,

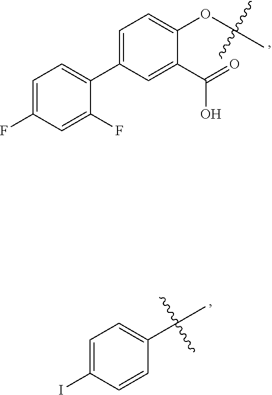 [SMILES: *Oc1ccc(-c2ccc(F)cc2F)cc1C(=O)O ; *c1ccc(I)cc1]

or a quaternary amine of any one of the preceding embodiments, for example, —N+(CH₃)₃.

### Numbered Embodiment 303

303. The cyclic peptide of any one of the preceding embodiments, wherein
(i) each R^{Y} and R^{Z} is independently selected from the group consisting of: hydrogen, optionally substituted (C₁-C₁₀) alkyl and optionally substituted (C₁-C₂₅) heteroalkyl, or
(ii) R^{Y} and R^{Z} come together with the N atom to which they are attached to form an optionally substituted (C₅0.7) heterocyclic ring or an optionally substituted (C₇-C₉) bicyclic heterocyclic ring.

### Numbered Embodiment 304

304. The cyclic peptide of any one of the preceding embodiments, wherein
(i) each R^{Y} and R^{Z} is independently selected from the group consisting of: hydrogen, optionally substituted (C₁-C₈) alkyl and optionally substituted (C₁-C₂₀) heteroalkyl, or
(ii) R^{Y} and R^{Z} come together with the N atom to which they are attached to form an optionally substituted (C) heterocyclic ring or an optionally substituted (C₈) bicyclic heterocyclic ring.

### Numbered Embodiment 305

305. The cyclic peptide of any one of the preceding embodiments, wherein R^{CT} is selected from the group consisting of: CONH2, CO(DiFPip), CO(Morph), CO(mPEG8), CO(NHPEG3a), CO(OAZBO), CO(TFMOHPip), CON(Me)2, CON(MePEG2), CON(mPEG2), CON(NMePip), CONH(PEG3a), CONH(PEG5a), CONHMe and CONMe2.

### Numbered Embodiment 306

306. The cyclic peptide of any one of the preceding embodiments, wherein R^{CT} is selected from the group consisting of: optionally substituted aminyl, optionally substituted piperidinyl, optionally substituted piperazinyl, optionally substituted morpholinyl and optionally substituted 3-oxa-8-azabicyclo[3.2.1]octanyl.

### Numbered Embodiment 307

307. The cyclic peptide of any one of the preceding embodiments, wherein R^{CT} is N(R′)(R^{Z}), wherein R^{Y} is hydrogen and R^{Z} is an optionally substituted (C₁-C₃₀) heteroalkyl that comprises a quaternary amine, for example, CO(NHPEG3a), CONH(PEG3a) or CONH(PEG5a).

### Numbered Embodiment 308

308. The cyclic peptide of any one of the preceding embodiments, wherein when R, and/or R^{Z} is an optionally substituted alkyl, each optionally substituted alkyl is independently unsubstituted or is substituted with one or more substituents independently selected from the group consisting of: a quaternary amine of any one of the preceding embodiments, for example, —N+(CH₃)₃.

### Numbered Embodiment 309

309. The cyclic peptide of any one of the preceding embodiments, wherein when R, and/or R^{Z} is an optionally substituted heteroalkyl, each optionally substituted heteroalkyl is independently unsubstituted or is substituted with one or more substituents independently selected from the group consisting of: a quaternary amine of any one of the preceding embodiments, for example, —N+(CH₃)₃.

### Numbered Embodiment 310

310. The cyclic peptide of any one of the preceding embodiments, wherein when R, and R^{Z} come together with the N atom to which they are attached to form an optionally substituted heterocyclic ring, for example, optionally substituted aminyl, optionally substituted piperidinyl, optionally substituted piperazinyl and optionally substituted morpholinyl, the optionally substituted heterocyclic ring is unsubstituted or is substituted with one or more substituents independently selected from the group consisting of: Fluoro, —OH, —CF₃, (C₁-C₃) alkyl, or a quaternary amine of any one of the preceding embodiments, for example, —N+(CH₃)₃.

### Numbered Embodiment 311

311. The cyclic peptide of any one of the preceding embodiments, wherein when R, and R^{Z} come together with the N atom to which they are attached to form an optionally substituted bicyclic heterocyclic ring, for example, optionally substituted 3-oxa-8-azabicyclo[3.2.1]octanyl, the optionally substituted bicyclic heterocyclic ring is unsubstituted or is substituted with one or more substituents independently selected from the group consisting of: Fluoro, —OH, —CF₃, (C₁-C₃) alkyl, or a quaternary amine of any one of the preceding embodiments, for example, —N+(CH₃)₃.

### Numbered Embodiment 312

312. The cyclic peptide of any one of the preceding embodiments, wherein the cyclic peptide further comprises one or more natural or unnatural polymer(s) or a combination(s) thereof.

### Numbered Embodiment 313

313. The cyclic peptide of any one of the preceding embodiments, wherein the cyclic peptide further comprises a natural or unnatural polymer or a combination thereof.

### Numbered Embodiment 314

314. The cyclic peptide of any one of the preceding embodiments, wherein the cyclic peptide further comprises one or more natural or unnatural polymer or a combination thereof that is conjugated to R^{NT}, when present, and/or R^{CT}, when present, and/or the amino acid residues at positions Z³, Z⁵, Z⁶, Z⁷, Z⁸, Z¹⁰, Z¹¹, Z¹², Z¹³, Z¹⁴, Z¹¹ and/or Z¹⁶, when an amino acid residue is present at the position.

### Numbered Embodiment 315

315. The cyclic peptide of any one of the preceding embodiments, wherein the cyclic peptide further comprises a natural or unnatural polymer or a combination thereof that is conjugated to R^{NT}, when present, or R^{CT}, when present, or the amino acid residues at positions Z³, Z⁵, Z⁶, Z⁷, Z⁸, Z¹⁰, Z¹¹, Z¹², Z¹³, Z¹⁴, Z¹⁵ or Z¹⁶, when an amino acid residue is present at the position.

### Numbered Embodiment 316

316. The cyclic peptide of any one of the preceding embodiments, wherein each natural or unnatural polymer(s) or a combination(s) thereof is/are independently one of the natural or unnatural polymer(s) or a combination(s) thereof disclosed in the description.

### Numbered Embodiment 317

317. The cyclic peptide of any one of the preceding embodiments, wherein the cyclic peptide is an inhibitor of an interleukin-23 (IL-23) receptor.

### Numbered Embodiment 318

318. The cyclic peptide of any one of the preceding embodiments, wherein the cyclic peptide inhibits the binding of an interleukin-23 (TL-23) to an TL-23 receptor.

### Numbered Embodiment 319

319. The cyclic peptide of any one of the preceding embodiments, wherein the cyclic peptide inhibits an interleukin-23 (TL-23) receptor to reduce or prevent the activation of the Jak-Stat signaling molecules, Jak2, Tyk2, Stat1, Stat 3, Stat 4, and/or Stat 5.

### Numbered Embodiment 320

320. The cyclic peptide of any one of the preceding embodiments, wherein the cyclic peptide inhibits an interleukin-23 (IL-23) receptor to reduce or prevent the phosphorylation of Stat 3.

### Numbered Embodiment 321

321. The cyclic peptide of any one of the preceding embodiments, wherein the cyclic peptide inhibits an interleukin-23 (IL-23) receptor in a reporter assay with an IC₅₀ value less than about 6.3 μM.

### Numbered Embodiment 322

322. The cyclic peptide of any one of the preceding embodiments, wherein the cyclic peptide inhibits an interleukin-23 (IL-23) receptor in a reporter assay with an IC₅₀ value less than about 2 μM.

### Numbered Embodiment 323

323. The cyclic peptide of any one of the preceding embodiments, wherein the cyclic peptide inhibits an interleukin-23 (IL-23) receptor in a reporter assay with an IC₅₀ value less than about 1 μM.

### Numbered Embodiment 324

324. The cyclic peptide of any one of the preceding embodiments, wherein the cyclic peptide inhibits an interleukin-23 (IL-23) receptor in a reporter assay with an IC₅₀ value less than about 0.5 μM.

### Numbered Embodiment 325

325. The cyclic peptide of any one of the preceding embodiments, wherein the cyclic peptide inhibits an interleukin-23 (IL-23) receptor in a reporter assay with an IC₅₀ value less than about 0.25 μM.

### Numbered Embodiment 326

326. The cyclic peptide of any one of the preceding embodiments, wherein the cyclic peptide inhibits an interleukin-23 (IL-23) receptor in a reporter assay with an IC₅₀ value less than about 0.1 μM.

### Numbered Embodiment 327

327. The cyclic peptide of any one of the preceding embodiments, wherein the cyclic peptide inhibits an interleukin-23 (IL-23) receptor in a reporter assay with an IC₅₀ value less than about 0.05 μM.

### Numbered Embodiment 328

328. The cyclic peptide of any one of the preceding embodiments, wherein the cyclic peptide inhibits an interleukin-23 (IL-23) receptor in a reporter assay with an IC₅₀ value less than about 0.025 μM.

### Numbered Embodiment 329

329. The cyclic peptide of any one of the preceding embodiments, wherein the cyclic peptide inhibits an interleukin-23 (IL-23) receptor in a reporter assay with an IC₅₀ value less than about 0.01 μM.

### Numbered Embodiment 330

330. The cyclic peptide of any one of the preceding embodiments, wherein the cyclic peptide inhibits an interleukin-23 (IL-23) receptor in an IL-23 reporter assay with an IC₅₀ value less than about 0.005 μM.

### Numbered Embodiment 331

331. The cyclic peptide of any one of the preceding embodiments, wherein the cyclic peptide inhibits an interleukin-23 (IL-23) receptor in a reporter assay with an IC₅₀ value less than about 0.0025 μM.

### Numbered Embodiment 332

332. The cyclic peptide of any one of the preceding embodiments, wherein the IL-23 reporter assay is as described in the description.

### Numbered Embodiment 333

333. The cyclic peptide of any one of the preceding embodiments, wherein the cyclic peptide inhibits an interleukin-23 (IL-23) receptor in a peripheral blood mononuclear cell (PBMC) pSTAT3 assay with an IC₅₀ value less than about 0.05 nM.

### Numbered Embodiment 334

334. The cyclic peptide of any one of the preceding embodiments, wherein the cyclic peptide inhibits an interleukin-23 (IL-23) receptor in a peripheral blood mononuclear cell (PBMC) pSTAT3 assay with an IC₅₀ value less than about 0.04 nM.

### Numbered Embodiment 335

335. The cyclic peptide of any one of the preceding embodiments, wherein the cyclic peptide inhibits an interleukin-23 (IL-23) receptor in a peripheral blood mononuclear cell (PBMC) pSTAT3 assay with an IC₅₀ value less than about 0.03 nM.

### Numbered Embodiment 336

336. The cyclic peptide of any one of the preceding embodiments, wherein the cyclic peptide inhibits an interleukin-23 (IL-23) receptor in a peripheral blood mononuclear cell (PBMC) pSTAT3 assay with an IC₅₀ value less than about 0.02 nM.

### Numbered Embodiment 337

337. The cyclic peptide of any one of the preceding embodiments, wherein the cyclic peptide inhibits an interleukin-23 (IL-23) receptor in a peripheral blood mononuclear cell (PBMC) pSTAT3 assay with an IC₅₀ value less than about 0.01 nM.

### Numbered Embodiment 338

338. The cyclic peptide of any one of the preceding embodiments, wherein the peripheral blood mononuclear cell (PBMC) pSTAT3 assay is as described in the description.

Numbered Embodiment 339. The cyclic peptide of any one of the preceding embodiments, wherein the cyclic peptide exhibits an Exposed Polar Surface Area (EPSA) of less than about 275 Å² in an EPSA assay.

Numbered Embodiment 340. The cyclic peptide of any one of the preceding embodiments, wherein the cyclic peptide exhibits an Exposed Polar Surface Area (EPSA) of less than about 250 Å² in an EPSA assay.

Numbered Embodiment 341. The cyclic peptide of any one of the preceding embodiments, wherein the cyclic peptide exhibits an Exposed Polar Surface Area (EPSA) of less than about 225 Å² in an EPSA assay.

Numbered Embodiment 342. The cyclic peptide of any one of the preceding embodiments, wherein the cyclic peptide exhibits an Exposed Polar Surface Area (EPSA) of less than about 200 Å² in an EPSA assay.

Numbered Embodiment 343. The cyclic peptide of any one of the preceding embodiments, wherein the cyclic peptide exhibits an Exposed Polar Surface Area (EPSA) of less than about 175 Å² in an EPSA assay.

Numbered Embodiment 344. The cyclic peptide of any one of the preceding embodiments, wherein the cyclic peptide exhibits an Exposed Polar Surface Area (EPSA) of less than about 150 Å² in an EPSA assay.

Numbered Embodiment 345. The cyclic peptide of any one of the preceding embodiments, wherein the cyclic peptide exhibits an Exposed Polar Surface Area (EPSA) of less than about 145 Å² in an EPSA assay.

Numbered Embodiment 346. The cyclic peptide of any one of the preceding embodiments, wherein the cyclic peptide exhibits an Exposed Polar Surface Area (EPSA) of less than about 140 Å² in an EPSA assay.

Numbered Embodiment 347. The cyclic peptide of any one of the preceding embodiments, wherein the Exposed Polar Surface Area (EPSA) assay is as described in the description.

Numbered embodiment 348.A compound which is selected from any one of the tables 1A, 1B, 1C, 1D, lE and 1 For pharmaceutically acceptable salts thereof or solvates thereof.

Numbered Embodiment 349.A pharmaceutical composition comprising the cyclic peptide or pharmaceutically acceptable salt thereof or solvate thereof of any one of the preceding embodiments, and a pharmaceutically acceptable carrier, excipient, or diluent.

Numbered Embodiment 350. The pharmaceutical composition of any one of the preceding embodiments, further comprising an enteric coating.

Numbered Embodiment 351. The pharmaceutical composition of any one of the preceding embodiments, wherein the enteric coating protects and releases the pharmaceutical composition within a subject's lower gastrointestinal system.

Numbered Embodiment 352. A method for treating an Inflammatory Bowel Disease (IBD), ulcerative colitis, Crohn's disease, Celiac disease (nontropicalSprue), enteropathy associated with seronegative arthropathies, microscopic colitis, collagenous colitis, eosinophilic gastroenteritis, colitis associated with radio- or chemo-therapy, colitis associated with disorders of innate immunity as in leukocyte adhesion deficiency-1, chronic granulomatous disease, glycogen storage disease type ib, Hermansky-Pudlak syndrome, Chediak-Higashi syndrome, and Wiskott-Aldrich Syndrome, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, psoriasis, psoriatic arthritis, or graft versus host disease in a subject, comprising providing to the subject an effective amount of the cyclic peptide or pharmaceutically acceptable salt thereof or solvate thereof of any one of the preceding embodiments, or the pharmaceutical composition of any one of the preceding embodiments.

Numbered Embodiment 353. The method of any one of the preceding embodiments, wherein the cyclic peptide or pharmaceutically acceptable salt thereof or solvate thereof or the pharmaceutical composition is provided to the subject by an oral, parenteral, intravenous, peritoneal, intradermal, subcutaneous, intramuscular, intrathecal, inhalation, vaporization, nebulization, sublingual, buccal, parenteral, rectal, intraocular, inhalation, topically, vaginal, or topical route of administration.

Numbered Embodiment 354. The method of any one of the preceding embodiments for treating Inflammatory Bowel Disease (IBD), ulcerative colitis, or Crohn's disease, wherein the cyclic peptide or pharmaceutically acceptable salt thereof or solvate thereof or the pharmaceutical composition is provided to the subject orally.

Numbered embodiment 355. The method of any one of the preceding embodiments for treating psoriasis, wherein the cyclic peptide or pharmaceutically acceptable salt thereof or solvate thereof or the pharmaceutical composition is provided to the subject orally, topically, parenterally, intravenously, subcutaneously, peritonealy, or intravenously.

Numbered Embodiment 356. The cyclic peptide of any one of the preceding embodiments, or the pharmaceutical composition of any one of the preceding embodiments for use in the treatment of an Inflammatory Bowel Disease (IBD), ulcerative colitis, Crohn's disease, Celiac disease (nontropical Sprue), enteropathy associated with seronegative arthropathies, microscopic colitis, collagenous colitis, eosinophilic gastroenteritis, colitis associated with radio- or chemo-therapy, colitis associated with disorders of innate immunity as in leukocyte adhesion deficiency-1, chronic granulomatous disease, glycogen storage disease type 1b, Hermansky-Pudlak syndrome, Chediak-Higashi syndrome, and Wiskott-Aldrich Syndrome, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, psoriasis, psoriatic arthritis, or graft versus host disease in a subject, comprising providing to the subject an effective amount of the cyclic peptide or pharmaceutically acceptable salt thereof or solvate thereof of any one of the preceding embodiments, or the pharmaceutical composition of any one of the preceding embodiments.

Numbered Embodiment 357. The cyclic peptide for use according to any one of the preceding embodiments or the pharmaceutical composition for use according to any one of the preceding embodiments, wherein the cyclic peptide or pharmaceutically acceptable salt thereof or solvate thereof or the pharmaceutical composition is provided to the subject by an oral, parenteral, intravenous, peritoneal, intradermal, subcutaneous, intramuscular, intrathecal, inhalation, vaporization, nebulization, sublingual, buccal, parenteral, rectal, intraocular, inhalation, topically, vaginal, or topical route of administration.

Numbered Embodiment 358. The cyclic peptide for use according to any one of the preceding embodiments or the pharmaceutical composition for use according to any one of the preceding embodiments, for use in the treatment of Inflammatory Bowel Disease (IBD), ulcerative colitis, or Crohn's disease, wherein the cyclic peptide or pharmaceutically acceptable salt thereof or solvate thereof or the pharmaceutical composition is provided to the subject orally.

Numbered Embodiment 359. The cyclic peptide or the pharmaceutical composition for use according to any one of the preceding embodiments, for use in the treatment of psoriasis, wherein the cyclic peptide or pharmaceutically acceptable salt thereof or solvate thereof or the pharmaceutical composition is provided to the subject orally, topically, parenterally, intravenously, subcutaneously, peritonealy, or intravenously.

Numbered Embodiment 360. The cyclic peptide of any one of the preceding embodiments, or the pharmaceutical composition of any one of the preceding embodiments for use as a medicament.

Numbered Embodiment 361. The use of the cyclic peptide of any one of the preceding embodiments, or the pharmaceutical composition of any one of the preceding embodiments for the manufacture of a medicament for treating a disease.

Numbered Embodiment 362. The use of the cyclic peptide of any one of the preceding embodiments, or the pharmaceutical composition of any one of the preceding embodiments for the manufacture of a medicament for treating a disease disclosed in any one of the preceding embodiments.

LENGTHY TABLES
The patent application contains a lengthy table section. A copy of the table is available in electronic form from the USPTO web site (https://seqdata.uspto.gov/docdetail?docId=US20260022143A1). An electronic copy of the table will also be available from the USPTO upon request and payment of the fee set forth in 37 CFR 1.19(b)(3).

What is claimed is:

1. A cyclic peptide, or a pharmaceutically acceptable salt or solvate thereof, comprising an amino acid sequence of Formula (A):
Z³-Z⁴-Z⁵-Z⁶-Z⁷-Z⁸-Z⁹-Z¹⁰-Z¹¹-Z¹²-Z¹³-Z¹⁴-
Z¹⁵-Z¹⁶ (A),
wherein:
the amino acid residue at position Z³ is absent or the residue of r;
the amino acid residue at position Z⁴ is the residue of an amino acid that is connected to the amino acid residue at position Z⁹;
the amino acid residue at position Z⁵ is the residue of N(N(Me)2);
the amino acid residue at position Z⁶ is the residue of T;
the amino acid residue at position Z⁷ is the residue of 7MeW;
the amino acid residue at position Z⁸ is the residue of K(NMeAc);
the amino acid residue at position Z⁹ is the residue of an amino acid that is connected to the amino acid residue at position Z⁴;
the amino acid residue at position Z¹⁰ is the residue of TMAPF;
the amino acid residue at position Z¹¹ is the residue of 2Nal;
the amino acid residue at position Z¹² is absent or the residue of THP;
the amino acid residue at position Z¹³ is the residue of K(NMeAc);
the amino acid residue at position Z¹⁴ is the residue of N;
the amino acid residue at position Z¹¹ is absent or the residue of 3Pya;
the amino acid residue at position Z¹⁶ is absent or the residue of Sar;
eight or fewer of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position; and
at least one of the amino acid residues at positions Z³, Z⁵, Z⁶, Z⁷, Z⁸, Z¹⁰, Z¹¹, Z¹², Z¹³, Z¹⁴, Z¹⁵ and/or Z¹⁶, when an amino acid residue is present at the position, each independently comprise(s) at least one quaternary amine.

2. A cyclic peptide, or a pharmaceutically acceptable salt or solvate thereof, comprising an amino acid sequence of Formula (B):
Z³-Z⁴-Z⁵-Z⁶-Z⁷-Z⁸-Z⁹-Z¹⁰-Z¹¹-Z¹²-Z¹³-Z¹⁴-
Z¹⁵-Z¹⁶ (B),
wherein:
the amino acid residue at position Z³ is absent or the residue of r;
the amino acid residue at position Z⁴ is the residue of an amino acid that is connected to the amino acid residue at position Z⁹;
the amino acid residue at position Z⁵ is the residue of N(N(Me)2);
the amino acid residue at position Z⁶ is the residue of T;
the amino acid residue at position Z⁷ is the residue of 7MeW;
the amino acid residue at position Z⁸ is the residue of K(NMeAc);
the amino acid residue at position Z⁹ is the residue of an amino acid that is connected to the amino acid residue at position Z⁴;
the amino acid residue at position Z⁰ is the residue of TMAPF;
the amino acid residue at position Z¹¹ is the residue of 2Nal;
the amino acid residue at position Z² is absent or the residue of THP;
the amino acid residue at position Z¹³ is the residue of K(NMeAc);
the amino acid residue at position Z⁴ is the residue of N;
the amino acid residue at position Z¹⁵ is absent or the residue of 3Pya;
the amino acid residue at position Z¹⁶ is absent or the residue of Sar;
eight or fewer of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position; and
at least one of the amino acid residues at positions Z³, Z⁵, Z⁶, Z⁷, Z⁸, Z¹⁰, Z¹¹, Z¹², Z¹³, Z¹⁴, Z¹⁵ and/or Z¹⁶, when an amino acid residue is present at the position, each independently comprise(s) at least one masked amine and/or masked amide.

3. The cyclic peptide of any one of the preceding claims, wherein the amino acid residue at position Z⁴ is the residue of Abu or the residue of an amino acid comprising a sulfhydryl group, optionally wherein the amino acid comprising a sulfhydryl group is selected from the group consisting of: Pen, C or aMeC.

4. The cyclic peptide of any one of the preceding claims, wherein the amino acid residue at position Z⁹ is the residue of an amino acid comprising a sulfhydryl group, optionally wherein the amino acid comprising a sulfhydryl group is selected from the group consisting of Pen, C or aMeC.

5. The cyclic peptide of any one of the preceding claims, wherein:
(a) when the amino acid residue at position Z is the residue of an amino acid comprising a sulfhydryl group, the amino acid residue at position Z⁴ is connected to the amino acid residue at position Z⁹ by a disulfide bond formed between the amino acid comprising a sulfhydryl group at position Z⁴ and the amino acid comprising a sulfhydryl group at position Z⁹; or
(b) the amino acid residue at position Z⁴ is the residue of an amino acid comprising a sulfhydryl group, and the amino acid residue at position Z⁴ is connected to the amino acid residue at position Z⁹ by a disulfide bond formed between the amino acid comprising a sulfhydryl group at position Z⁴ and the amino acid comprising a sulfhydryl group at position Z⁹; or
(c) when the amino acid residue at position Z⁴ is the residue of Abu, the amino acid residue at position Z⁴ is connected to the amino acid residue at position Z⁹ by a thioether bond formed between the Abu at position Z⁴ and the amino acid comprising a sulfhydryl group at position Z⁹; or
(d) the amino acid residue at position Z⁴ is the residue of Abu, and the amino acid residue at position Z⁴ is connected to the amino acid residue at position Z⁹ by a thioether bond formed between the Abu at position Z⁴ and the amino acid comprising a sulfhydryl group at position Z⁹.

6. The cyclic peptide of any one of the preceding claims, wherein the cyclic peptide further comprises R^{NT}, wherein R^{NT} is bound to the N-terminal amine of the amino acid residue at position
(i) Z³ when Z³ is present, or
(ii) Z⁴ when Z³ is absent; and
R^{NT} is selected from the group consisting of: —C(O)-optionally substituted (C₁-C₂₀) alkyl and —C(O)-optionally substituted (C₁-C₄₀) heteroalkyl.

7. The cyclic peptide of any one of the preceding claims, wherein the cyclic peptide further comprises R^{CT}, wherein R^{CT} is bound to the carbonyl derived from the C-terminal carboxylic acid of the amino acid residue at position
(i) Z¹⁶ when Z¹⁶ is present,
(ii) Z¹¹ when Z¹⁶ is absent, or
(iii) Z¹⁴ when Z¹¹ and Z¹⁶ are absent; and
R^{C}r is —N(R^{y})(R^{Z}), wherein
(i) each Rⁱ and R^{Z} is independently selected from the group consisting of hydrogen, optionally substituted (C₁-C₁₅) alkyl and optionally substituted (C₁-C₃₀) heteroalkyl, or
(ii) each R^{Y} and R^{Z} come together with the N atom to which they are attached to form an optionally substituted (CH₃—C₁₄) heterocyclic ring or an optionally substituted (C₅-C₁₀) bicyclic heterocyclic ring.

8. The cyclic peptide of any one of the preceding claims, wherein four or fewer of the amino acid residues at positions Z³, Z⁵, Z⁶, Z⁷, Z⁸, Z¹⁰, Z¹¹, Z¹², Z¹³, Z¹⁴, Z¹⁵ and Z¹⁶, when an amino acid residue is present at the position, are independently replaced with an amino acid residue that is different from the amino acid residue recited at said position.

9. The cyclic peptide of any one of the preceding claims, wherein at least one of the amino acid residues at positions Z³, Z⁵, Z⁶, Z⁷, Z⁸, Z¹⁰, Z¹¹, Z¹², Z¹³, Z¹⁴, Z¹⁵ and/or Z¹⁶, when an amino acid residue is present at the position, each independently comprise(s) at least one quintenary amine.

10. The cyclic peptide of any one of the preceding claims, wherein each quintenary amine is independently selected from the group consisting of: 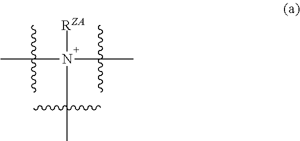
wherein each R^{ZA} is independently selected from the group consisting of optionally substituted (C₁-C₂₀) alkyl and optionally substituted (C₁-C₂₀) heteroalkyl; 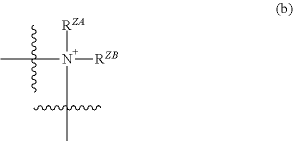
wherein each R^{ZA} and R^{Z}B is independently selected from the group consisting of: optionally substituted (C₁-C₂₀) alkyl and optionally substituted (C₁-C₂₀) heteroalkyl; 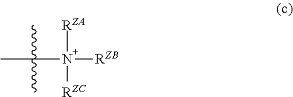
R wherein
(c)(i) each R^{ZA}, R^{Z}B and R^{Z}C is independently selected from the group consisting of: optionally substituted (C₁-C₂₀) alkyl and optionally substituted (C₁-C₂₀) heteroalkyl,
(c)(ii) each R^{ZA} is independently selected from the group consisting of optionally substituted (C₁-C₂₀) alkyl and optionally substituted (C₁-C₂₀) heteroalkyl, and each R^{ZB} and R^{ZC} come together with the N atom to which they are attached to form an optionally substituted (CH₃—C₁₄) heterocyclic ring, or
(c)(iii) each W^{A}, R^{ZB} and R^{ZC} come together with the N atom to which they are attached to form an optionally substituted (C₅-C₁₀) bicyclic heterocyclic ring; 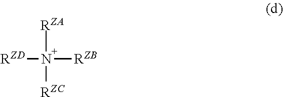
wherein
(d)(i) each R^{ZA} and R^{ZB} is independently selected from the group consisting of optionally substituted (C₁-C₂₀) alkyl and optionally substituted (C₁-C₂₀) heteroalkyl, and each R^{ZC} and R^{ZD} come together with the N atom to which they are attached to form an optionally substituted (CH₃—C₁₄) heterocyclyl, or
(d)(ii) each R^{ZA} and R^{ZB} come together with the N atom to which they are attached to form an optionally substituted (CH₃—C₁₄) heterocyclyl, and each R^{ZC} and R^{ZD} come together with the N atom to which they are attached to form an optionally substituted (CH₃—C₁₄) heterocyclyl, or
(d)(iii) each R^{ZA} and R^{ZB} come together with the N atom to which they are attached to form an optionally substituted (CH₃—C₁₄) heterocyclyl, and each R^{ZC} and R^{ZD} come together with the N atom to which they are attached to form an optionally substituted (CH₃—C₁₄) heterocyclic ring; 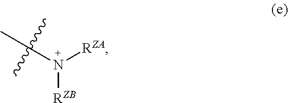
wherein each R^{ZA} and R^{ZB} come together with the N atom to which they are attached to form an optionally substituted (C₅-C₁₄) heteroaromatic ring, and 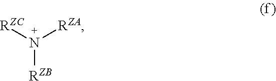
wherein each R^{ZA} is independently selected from the group consisting of: optionally substituted (C₁-C₂₀) alkyl and optionally substituted (C₁-C₂₀) heteroalkyl, and each R^{ZB} and R^{Z}C come together with the N atom to which they are attached to form an optionally substituted (C₅-C₁₄) heteroaryl.

11. The cyclic peptide of any one of the preceding claims, wherein at least one of the amino acid residues at positions Z³, Z⁵, Z⁶, ⁷, Z⁸, Z¹⁰, Z¹¹, Z¹², Z¹³, Z¹⁴, Z¹⁵ and/or Z¹⁶, when an amino acid residue is present at the position, each independently comprise(s) at least one masked amine and/or masked amide.

12. The cyclic peptide of any one of the preceding claims, wherein at least one of the masked amine(s) and/or masked amide(s) is independently selected from the group consisting of: 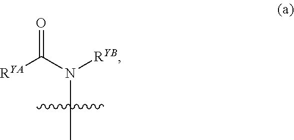
wherein each R^{YA} is independently selected from the group consisting of: optionally substituted (C₁-C₁₀) alkyl and optionally substituted (C₁-C₂₀) heteroalkyl, and each R^{YB} is independently selected from the group consisting of hydrogen, optionally substituted (C₁-C₁₀) alkyl and optionally substituted (C₁-C₂₀) heteroalkyl; 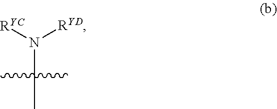
wherein each R^{YC} is independently selected from the group consisting of: optionally substituted (C₁-C₁₀) alkyl and optionally substituted (C₁-C₂₀) heteroalkyl, and each R^{YD} is independently selected from the group consisting of: hydrogen, optionally substituted (C₁-C₁₀) alkyl and optionally substituted (C₁-C₂₀) heteroalkyl; 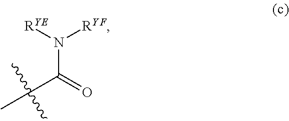
wherein each Rⁱ is independently selected from the group consisting of: optionally substituted (C₁-C₁₀) alkyl and optionally substituted (C₁-C₂₀) heteroalkyl, and each Rⁱ is independently selected from the group consisting of: hydrogen, optionally substituted (C₁-C₁₀) alkyl and optionally substituted (C₁-C₂₀) heteroalkyl; and 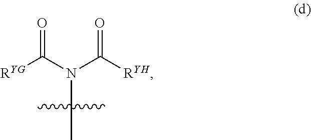
wherein each R^{YG} and R^{YH} is independently selected from the group consisting of: optionally substituted (C₁-C₁₀) alkyl and optionally substituted (C₁-C₂₀) heteroalkyl.

13. The cyclic peptide of any one of the preceding claims, wherein:
(a) when the amino acid residue at position Z³ is replaced with an amino acid residue that is different from the amino acid residue recited at said position, the amino acid residue at position Z³ is replaced with an amino acid residue selected from the group consisting of APEG2ser, APEG2Ser, APEG2Ser(S*), e(c), e(C), hk(Me)₃, k(5cpa), k(cPEG3a), k(d), k(D), k(dPEG12Ac), k(dPEG6Ac), k(dPEG9Ac), k(Me)₃, K(Me)₃, k(PEG2PEG2gEC12), k(PEG2PEG2gEC14), k(PEG2PEG2PEG2PEG2gEC12), k(PEG2PEG6gEC12), SP6, APEG2Ser(RS), gPEG2Ser and k(PEG2PEG2gE(c)C12; and/or
(b) when the amino acid residue at position Z⁵ is replaced with an amino acid residue that is different from the amino acid residue recited at said position, the amino acid residue at position Z⁵ is replaced with an amino acid residue selected from the group consisting of A, APEG2Ser(S*), Dab(Me)₃, F, Gab, K(cPEG3a), K(Me)₃, K(PEG2PEG2gEC12), K(PEG2PEG2gEC14), L, N, N(N(Me)), N(NMe), Q and W; and/or
(c) when the amino acid residue at position Z⁶ is replaced with an amino acid residue that is different from the amino acid residue recited at said position, the amino acid residue at position Z⁶ is replaced with an amino acid residue selected from the group consisting of A and L; and/or
(d) when the amino acid residue at position Z⁷ is replaced with an amino acid residue that is different from the amino acid residue recited at said position, the amino acid residue at position Z⁷ is replaced with an amino acid residue selected from the group consisting of W, 7(3NAcPh)W, 7CF3W, NMe7MeW, 2Nal, A, F and L; and/or
(e) when the amino acid residue at position Z⁸ is replaced with an amino acid residue that is different from the amino acid residue recited at said position, the amino acid residue at position Z⁸ is replaced with an amino acid residue selected from the group consisting of K(NMeAC), Q, 4AmPhe, A, AIB, APEG2Ser, APEG2Ser(R*), APEG2Ser(S*), Cit, Dab(NMeAc), Dab(NMecam), Dab(NMeCam), Dab(NMeCOmPEG6), Dab(NMecPEG2a), Dab(NMecPEG3a), Dab(NMecPEG5a), Dap(NMeAc), F, K(4cpg), K(Ac), K(cPEG3a), K(Me)₃, K(NMeCOmPEG6), K(NMePEG3a), K(NmPEG6Ac), K(PEG2PEG2gEC12), K(PEG2PEG2gEC14), L, Paf(Ac), Q(N(Me)2), Q(NHtBu), W, Y and K(cPEG3aCO); and/or
(f) when the amino acid residue at position Z¹⁰ is replaced with an amino acid residue that is different from the amino acid residue recited at said position, the amino acid residue at position Z¹⁰ is replaced with an amino acid residue selected from the group consisting of:
AEF, 4DMPEF, 4DMPzEF, 4TMABYF, ACHMF, AEF((Ch)cPEG3a), AEF(Ac), AEF(AcCh), AEF(aPEG2a), AEF(BisMEP), AEF(BisMEPa), AEF(BisPEG2a)(RS), AEF(BisPEG2a)(S*),
AEF(G), AEF(Me)2, AEF(MEP), AEF(MePrpa), AEF(N(Me)2), AEF(NHCh), AEF(NHcPEG3a), AEF(NMe), AEF(NMe2mPEG3), AEF(NMe3), AEF(NMeBismPEG3), AEF(NMePEG2a), AEF(NmPEG6), AEF(NsCh), AEF(PEG2a), AEF(SPD), APEG2F, APEG3F, dFPPEG3F, Diazabiclyclooctane6F, DMMMF, DMPMF, DMTASF, F(4G), F(4N3), F(4TzLDMA4mPEG), F(4Tz1MME), F(4TzLMMol), F(4TzlMMo3), F(4TzlMMo4), F(4TzLTMA1), F(4TzlTMA2), F(4TzlTMA3), F(4TzlTMA4), F(4TzlTMA5), GPEG3F, hFTMAPF, MMoEF, MMoPF, MMPEG3F, morfTMA4F, mPEG2TMA2F, mPEG2TMA4F,
mPEG3TMA4F, MPzPEG3F, MTASF, NPyEF, NPyPEG3F, PiperazinequatF, TBAPEG3F, TMA3F, TMA4F, TMA6F, TMA8F, Tzl(Ch), TzlChmPEG, TzlChmPEG3, Y(C9OH), Y(OEOXIMECh), Y(OTzlCh), Y(OTzlChC16), Y(OTzlChC8), Y(OTzLC1aC8), Y(OTzChmPEG), Y(OTzChmPEG3), Y(OTzIPEG3a), Y(OTAPEG4a), Y(OTzITMA4), Y(OZOXIMECh), YC8CO(NHPEG3a), YC8COPip, YCF2H, ACHMF(R*, S*), ACHMF(S*, S*), AEF(cPEG3a), APF, F(4TzlAme2), F(4TzLG2), F(4TzlMMo7) and F(4TzLTMA7); and/or
(g) when the amino acid residue at position Z¹¹ is replaced with an amino acid residue that is different from the amino acid residue recited at said position, the amino acid residue at position Z¹¹ is replaced with an amino acid residue selected from the group consisting of A, F, L and W; and/or
(h) when the amino acid residue at position Z² is replaced with an amino acid residue that is different from the amino acid residue recited at said position, the amino acid residue at position Z² is replaced with an amino acid residue selected from the group consisting of: A, Achx, Achx(diF), Acpx, Aib, AIB, aMeK, aMeL, Chg, diFCpx, F, L, Pip(NMe), Pip(NMe2), W and diFAchx; and/or
(i) when the amino acid residue at position Z¹³ is replaced with an amino acid residue that is different from the amino acid residue recited at said position, the amino acid residue at position Z¹³ is replaced with an amino acid residue selected from the group consisting of:
K(NMeAC), E, A, AIB, aMeE, APEG2Ser, APEG2Ser(S*), Cit, Dab(NMeAc), Dab(NMecam), Dab(NMeCOmPEG6), Dab(NMecPEG2a), Dab(NMecPEG3a), Dap(Ac), Dap(NMeAc), E(c), E(C), F, K(5cpa), K(Ac), K(cPEG3a), K(d), K(D), K(dPEG12Ac), K(dPEG6Ac), K(dPEG9Ac), K(Me)₃, K(NMeCOmPEG6), K(NMeCOPEG4N+Me3), K(NMePEG3a), K(NmPEG6Ac), K(PEG2PEG2gEC12), K(PEG2PEG2gEC14), K(PEG2PEG2PEG2gEC12), L, Q(N(Me)2), Tetrazole, Tetrazole(NMe), W, K(DFN), K(IPB) and Nle; and/or
(j) when the amino acid residue at position Z¹⁴ is replaced with an amino acid residue that is different from the amino acid residue recited at said position, the amino acid residue at position Z¹⁴ is replaced with an amino acid residue selected from the group consisting of:
K(Ac) and N(NMe); and/or
(k) when the amino acid residue at position Z¹⁵ is replaced with an amino acid residue that is different from the amino acid residue recited at said position, the amino acid residue at position Z¹⁵ is replaced with an amino acid residue selected from the group consisting of:
3pya, 5CF33Pya, SMePyridinAla, bAla, dK, dL, F, f, H, h, k, N, NMe3Pya, NMebAla, NMeDTyr, om, Paf, s, t, THP, v, y and A; and/or
(l) when the amino acid residue at position Z¹⁶ is replaced with an amino acid residue that is different from the amino acid residue recited at said position, the amino acid residue at position Z¹⁶ is replaced with an amino acid residue selected from the group consisting of:
4diFPro, NMeDTyr, NMeK(PEG2PEG2C12), NMeK(PEG2PEG2C14), NMeK(PEG2PEG2gEC12), NMeK(PEG2PEG2gEC14), NMeK(PEG2PEG2K(PEG2PEG2gEC12)₂), NMeK(PEG2PEG2K(PEG2PEG2PEG2PEG2gEC12)₂), NMeK(PEG2PEG2PEG2gEC12), NMeK(PEG2PEG2PEG2PEG2gEC12), NMeK(PEG2PEG6gEC12), NMeK(PEG2PEG6gEC14), NMeK(SP6PEG2PEG2C12) and NMeK(SP6PEG2PEG2gEC12).

14. A compound which is selected from any one of the tables 1A, 1B, 1C, 1D, 1E and 1F or pharmaceutically acceptable salts thereof or solvates thereof.

15. A pharmaceutical composition comprising the cyclic peptide or pharmaceutically acceptable salt thereof or solvate thereof of any one of the preceding claims, and a pharmaceutically acceptable carrier, excipient, or diluent.

16. A method for treating an Inflammatory Bowel Disease (IBD), ulcerative colitis, Crohn's disease, Celiac disease (nontropical Sprue), enteropathy associated with seronegative arthropathies, microscopic colitis, collagenous colitis, eosinophilic gastroenteritis, colitis associated with radio- or chemo-therapy, colitis associated with disorders of innate immunity as in leukocyte adhesion deficiency-1, chronic granulomatous disease, glycogen storage disease type ib, Hermansky-Pudlak syndrome, Chediak-Higashi syndrome, and Wiskott-Aldrich Syndrome, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, psoriasis, psoriatic arthritis, or graft versus host disease in a subject, comprising providing to the subject an effective amount of the cyclic peptide or pharmaceutically acceptable salt thereof or solvate thereof of any one of the preceding claims, or the pharmaceutical composition of any one of the preceding claims.
